(19) United States
(12) Patent Application Publication
(10) Pub. No.: US 2025/0296992 A1
(43) Pub. Date: Sep. 25, 2025

(54) SLITRK6 BINDING AGENTS, CONJUGATES THEREOF AND METHODS OF USING THE SAME

(71) Applicant: Genmab A/S, Valby (DK)

(72) Inventors: Wanwan Shen, Suzhou (CN); Yang Xiao, Suzhou (CN); Haidong Liu, Suzhou (CN); Lei Wang, Suzhou (CN); Xuan Qiu, Suzhou (CN); Baiteng Zhao, Bellevue, WA (US); Zhu Chen, Woodinville, WA (US)

(21) Appl. No.: 19/016,173

(22) Filed: Jan. 10, 2025

Related U.S. Application Data
(60) Provisional application No. 63/619,728, filed on Jan. 10, 2024.

(30) Foreign Application Priority Data
Apr. 4, 2024 (WO) PCT/CN2024/086162

(51) Int. Cl. C07K 16/18; A61K 47/68; A61P 35/00; C07K 16/42

(52) U.S. Cl. CPC C07K 16/18; A61K 47/6803; A61K 47/6889; A61P 35/00; C07K 16/4241; C07K 2317/35; C07K 2317/52; C07K 2317/565; C07K 2317/77; C07K 2317/92

(57) ABSTRACT

The present invention provides SLITRK6 antibodies, antigen binding portions thereof, other binding agents and SLITRK6 conjugates thereof, as well as methods and uses of such antibodies and conjugates the treatment of cancer and autoimmune disease.

### CROSS REFERENCE

This patent application claims the benefit of U.S. Provisional Application No. 63/619,728, filed Jan. 10, 2024, and of International Application No. PCT/CN2024/086162, filed Apr. 4, 2024, each of which is hereby incorporated by reference in its entirety.

### SEQUENCE LISTING

The instant application contains a Sequence Listing which has been submitted electronically in XML format and is hereby incorporated by reference in its entirety. Said XML copy, created on Jan. 17, 2025, is named GMI-412_SequenceListing.xml and is 76 kilobytes in size.

### TECHNICAL FIELD

The present disclosure generally relates to antibodies and antibody-drug conjugates, as well as methods of using the antibodies and antibody-drug conjugates, and in particular, to such antibodies, antibody-drug conjugates, and methods related to SLITRK6-expressing diseases and disorders.

### BACKGROUND

A great deal of interest has surrounded the use of monoclonal antibodies (mAbs) for the targeted delivery of cytotoxic agents to cells associated with disease, such as cancer cells and other cells, in the form of antibody drug conjugates (or ADCs). The design of antibody drug conjugates, by attaching a cytotoxic agent, immune modulatory agent or other agent (collectively a “drug”) to an antibody, typically via a linker, involves consideration of a variety of factors. These factors include the identity and location of the chemical group for attachment of the drug, the mechanism of drug release, the structural element(s) (if any) providing release of the drug, and structural modification of the released free drug, if any. If the drug is released in the extracellular environment, the released form of the drug must be able to reach its target. If the drug is to be released after antibody drug conjugate internalization, the structural elements and mechanism of drug release must be consonant with the intracellular trafficking of the conjugate.

Another important factor in the design of antibody drug conjugates is the amount of drug that can be delivered per targeting agent (i.e., the number of drugs attached to each targeting agent (e.g., an antibody), referred to as the drug load or drug loading). Historically, assumptions were that higher drugs loads were superior to lower drug loads (e.g., 8-loads vs 4-loads). The rationale was that higher loaded conjugates would deliver more drug (e.g., cytotoxic agent) to the target cells. This rationale was supported by the observations that conjugates with higher drug loadings were more active against cell lines in vitro. Certain later studies revealed, however, that this assumption was not confirmed in animal models. Conjugates having drug loads of 4 or 8 of certain auristatins were observed to have similar activities in mouse models.

See, e.g., Hamblett et al., Clinical Cancer Res. 10:7063-70 (2004). Hamblett et al. further reported that the higher loaded ADCs were cleared more quickly from circulation in animal models. This faster clearance suggested a PK liability for higher loaded species as compared to lower loaded species. See Hamblett et al. In addition, higher loaded conjugates had lower maximum tolerated doses (MTDs) in mice, and as a result had narrower reported therapeutic indices. Id. In contrast, ADCs with a drug loading of 2 at engineered sites in a monoclonal antibody were reported to have the same or better PK and therapeutic indices as compared to certain 4-loaded ADCs. For example, see Junutula et al., Clinical Cancer Res. 16:4769 (2010). Thus, recent trends are to develop ADCs with low drug loadings.

An attractive target for cancer therapies employing ADCs is SLIT and NTRK-like protein 6 (SLITRK6). SLITRK6 protein is engaged in tight control of developmental processes, such as neurite outgrowth and modulation, cellular differentiation, and hormonal regulation. While SLITRK6 is found to be highly expressed in various cancers, such as BLCA, BRCA, HNSC, LUCA and GBMLGG, it has limited expression in normal tissues. The different expression between the cancer tissues and normal tissues makes it a promising tumor associated antigen. (Mol Cell Neurosci 2003; 24:117-129. Gene 2003; 315:87-94. Uniprot.) However, the pace for constructing effective SLITRK6 antibodies and related conjugates has been slow and the clinical trials with SLITRK6 antibodies and SLITRK6 ADCs have met with limited success thus far.

There is a need, therefore, for SLITRK6 antibodies generally, and for SLITRK6 ADCs in particular that allow for higher drug loading, but that maintain other characteristics of lower loaded conjugates, such as favorable PK properties. Embodiments of the present invention address these and related needs.

### SUMMARY

Provided herein are SLITRK6 binding agents, antibody drug conjugates (ADCs), and methods of using the binding agents and ADC to treatment diseases such as but not limited to cancers and autoimmune diseases.

In some embodiments, provided is a binding agent that includes a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of: SEQ ID NO: 3, SEQ ID NO: 4, SEQ ID NO: 5, SEQ ID NO: 6, SEQ ID NO: 7, and SEQ ID NO: 8, respectively; SEQ ID NO: 11, SEQ ID NO: 12, SEQ ID NO: 13, SEQ ID NO: 14, SEQ ID NO: 15 and SEQ ID NO: 16, respectively; SEQ ID NO: 19, SEQ ID NO: 20, SEQ ID NO: 21, SEQ ID NO: 22, SEQ ID NO: 23 and SEQ ID NO: 24, respectively; SEQ ID NO: 27, SEQ ID NO: 28, SEQ ID NO: 29, SEQ ID NO: 30, SEQ ID NO: 31 and SEQ ID NO: 32, respectively; and SEQ ID NO: 35, SEQ ID NO: 36, SEQ ID NO: 37, SEQ ID NO: 38, SEQ ID NO: 39 and SEQ ID NO: 40, respectively.

In some embodiments, provided is a binding agent (e.g., an antibody or antigen-binding portion thereof) comprising a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively.

In some embodiments, the binding agent (e.g., an antibody or antigen-binding portion thereof) comprises a heavy chain constant region comprising the amino acid sequence of SEQ ID NO: 49, 51, or 52. In some embodiments, the binding agent (e.g., an antibody or antigen-binding portion thereof) comprises a light chain constant region comprising the amino acid sequence of SEQ ID NO: 50. In some embodiments, the binding agent (e.g., an antibody or antigen-binding portion thereof) comprises a heavy chain constant region comprising the amino acid sequence of SEQ ID NO: 49, 51, or 52, and a light chain constant region comprising the amino acid sequence of SEQ ID NO: 50.

In some embodiments, provided is a binding agent (e.g., an antibody or antigen-binding portion thereof) comprising a heavy chain and a light chain comprising amino acid sequences set forth in SEQ ID NOs: 53 and 55, respectively. In some embodiments, provided is a binding agent (e.g., an antibody or antigen-binding portion thereof) comprising a heavy chain and a light chain comprising amino acid sequences set forth in SEQ ID NOs: 54 and 55, respectively.

In some embodiments, provided herein is a pharmaceutical composition comprising the binding agent of the present disclosure and a pharmaceutically acceptable carrier.

In some embodiments, provided herein is a nucleic acid encoding the binding agent of the present disclosure.

In some embodiments, provided herein is a vector comprising the nucleic acid of the present disclosure.

In some embodiments, provided herein is a cell line comprising the binding agent, the vector, or the nucleic acid of the present disclosure.

In some embodiments, provided herein is a conjugate that comprises the binding agent, at least one linker attached to the binding agent; at least one drug unit, wherein each drug unit is attached to a linker, wherein the linker optionally comprises at least one polar group.

In some embodiments, for the conjugate of the present disclosure, the linker is derived from a linker compound, or a stereoisomer or salt thereof, and the linker compound comprises: a linker unit; a stretcher group connected to the linker unit; an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher group and an attachment site to the linker unit; and the linker unit has an attachment site to the amino acid unit (when present) or to the stretcher group and to the at least one drug unit.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein the polymer unit comprises the formula:

˜-R⁰—(R³—R¹—[O—CH₂—CH₂]ₙ₀—R⁶—([O—CH₂—CH₂]ₙ₀—R²—R³—(NR⁴R⁵)ₙ₁)ₙ₂)ₙ₃ (Ia)
or a stereoisomer or salt thereof, wherein:
   R⁰ is a functional group for attachment to a subunit of the amino acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}— C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is selected from:

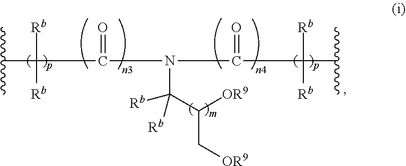

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
each v is independently 1-6, and
   n² is 1;

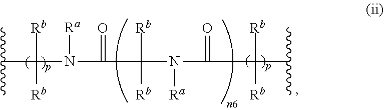

, wherein:
   each R^{a} is independently H or C₁₋₆ alkyl,
   each R^{b} is independently H or C₁₋₆ alkyl,
   n⁶ is 1-10,
   each p is independently 0-6, and
n² is 1;

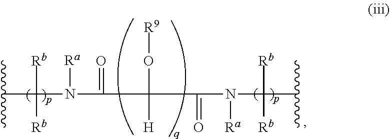

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
each v is independently 1-6, and
   n² is 1;

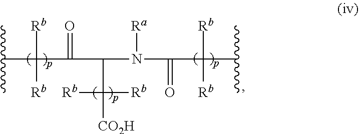

wherein:
   each R^{a} is independently H or C₁₋₆ alkyl,
   each R^{b} is independently H or C₁₋₆ alkyl,
   each p is independently 0-6, and
n² is 1;
(v) —R¹⁰—[O—CH₂—CH₂]₁₋₈—R¹⁰—, wherein:
   each R^{b} is independently H or C₁₋₆ alkyl,
   each R¹⁰ is independently

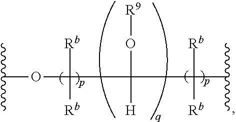

each p is independently 1-6,
   each R⁹ is independently H, acetyl, —P(═O)(OH)₂, or —(CH₂)ᵥ—O—S(═O)₂(OH), and
q is 1-8;
n² is 1; and
(vi) —N—(R¹—X—R²—)₂, wherein:
   each X is independently —NR^{a}—C(O)— or —C(O)NR^{a}—, and
   n² is 2; and
   the wavy line (˜) indicates the attachment site of the amino acid unit to R⁰;
   each n⁰ is independently 2-26;
   each n¹ is independently 1-6; and
   n³ is 1-6.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

˜R⁰—(R³—R¹—[O—CH₂—CH₂]ₙ₀—R²—(NR⁴R⁵)ₙ₁)ₙ₃ (Ia′)
or a stereoisomer or salt thereof, wherein:
   R⁰ is a functional group for attachment to a subunit of the amino acid unit;
   each R¹ and R² are independently a bond or C₁-C₆ alkylene;
   each R³ is independently —N(polyhydroxyl group)-, triazolyl, —C₁-C₁₂ alkylene-triazolyl-,

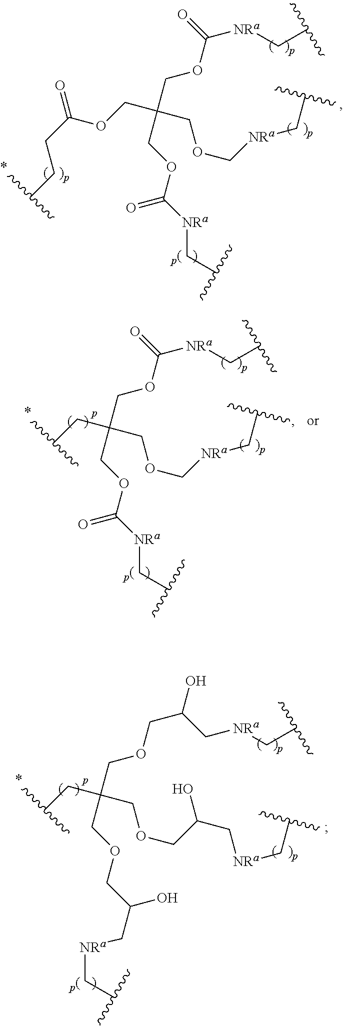

each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

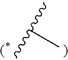

indicates the attachment site of R³ to R⁰
the wavy line

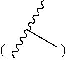

indicates the attachment site of the R³ to R¹;
each p is 1-6;
each n⁰ is independently 2-8;
each n¹ is independently 1-6; and
n³ is 1-6.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

˜R⁰—(R¹—[O—CH₂—CH₂]ₙ₀—R²—R³—(NR⁴R⁵)ₙ₁)ₙ₃ (Ia″)
or a stereoisomer or salt thereof, wherein:
   (i) R⁰ is a functional group for attachment to a subunit of the amino acid unit; each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
the wavy line (˜) indicates the attachment site of the amino acid unit to R⁰;
n⁰ is independently 2-26;
n¹ is 1-6; and
n³ is 1-6;
   (ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
n⁰ is 6;
n¹ is 1-6; and
n³ is 1;
   (iii) R⁰ is a functional group for attachment to a subunit of the amino acid unit;
R¹ and R² are each, independently, a bond or C₁-C₆ alkylene;
R³ is —NRₐ—C(O)—C₁-C₁₂ alkylene-C(O)—, wherein the alkylene is substituted with —SO₃H; R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
each n⁰ is independently 1-26;
n¹ is 1-6; and
n³ is 1-6; or
   (iv) R⁰ is

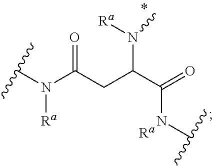

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R^{a} is independently H or C₁₋₆ alkyl;
the wavy line

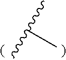

indicates the attachment site of R⁰ to the remainder of the polymer unit;
the wavy line (˜*) indicates the attachment site of the amino acid unit to R⁰;
n⁰ is 1-8;
n¹ is 1-6; and
n³ is 2.

In some embodiments, for the conjugate of the present disclosure, the linker compound comprises:
(a) a linker unit having from 1 to 4 attachment sites for a drug unit, said linker unit comprising a moiety of formula:

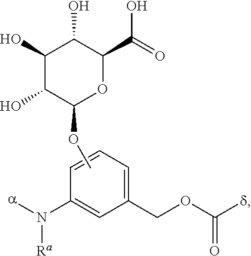

or a stereoisomer or salt thereof, wherein:
   α—represents a direct or indirect attachment site to an amino acid unit;
      δ—represents an attachment site to at least one of the drug units or for a linking group attached to the at least one of the drug units; and
R^{a} is H or C₁₋₆ alkyl;
(b) the amino acid unit having from 1 to 12 amino acid subunits; and
(c) at least one polar group attached to the amino acid unit, wherein the polar group comprises a polymer unit, optionally a sugar unit, and optionally a carboxyl unit.

In some embodiments, for the conjugate of the present disclosure, the linker compound comprises:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises:
   (i) an optionally substituted polyamide comprising the formula

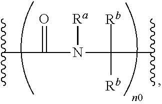

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

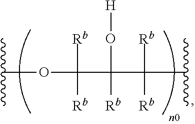

or a stereoisomer thereof, wherein each R^{b} is independently H or C₁₋₆ alkyl, and n⁰ is independently 2-26; or
(iii) combinations thereof.

In some embodiments, for the conjugate of the present disclosure, the linker compound comprises:
(a) the linker unit, which has from 1 to 4 attachment sites for the drug units and having one of the following structures (i) or (ii):

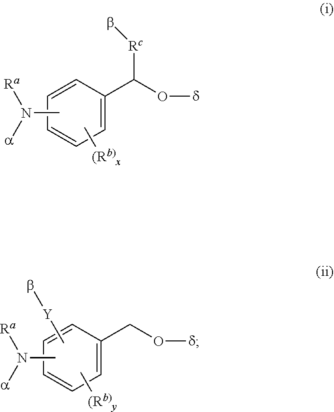

(b) the at least one polar group, each comprises a polymer unit, and
(c) the stretcher group, which has an attachment site for the binding agent; wherein:
α—is an attachment site to an enzyme-cleavable group;
   β—is an attachment site to the at least one polar group;
   δ—is H, an attachment site to at least one of the drug units, or an attachment site to a linking group attached to the at least one of the drug units;
   the polymer unit comprises a polyamide, a polyether, or a combination thereof, wherein the polyether comprises a hydroxyl group, a polyhydroxyl group, a sugar group, a carboxyl group, or combinations thereof;
   each R^{a} independently is H or C₁-C₆ alkyl;
   each R^{b} independently is halo, C₁₋₆ alkyl, an attachment site to at least one of the drug units, or an attachment site to at least one of the polar groups;
   x is 0, 1, 2, 3 or 4;
   y is 0, 1, 2 or 3;
   R^{c} is a bond, —C(O)—, —S(O)—, —SO₂—, C₁₋₆ alkylene, C₁₋₆ alkynylene, triazolyl or combinations thereof; and
   Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl or combinations thereof.

In some embodiments, provided herein is a pharmaceutical composition comprising the conjugate of the present disclosure and a pharmaceutically acceptable carrier.

In some embodiments, provided herein is a method of treating a SLITRK6+ cancer, comprising administering to a subject in need thereof a therapeutically effective amount of the binding agent, the pharmaceutical composition, the conjugate, or the pharmaceutical composition of the present disclosure.

In some embodiments, provided herein is a use of the conjugate or the pharmaceutical composition of the present disclosure for the treatment of SLITRK6+ cancer in a subject.

Additional features will be set forth in part in the description which follows, and in part will become apparent to those skilled in the art upon examination of the following and the accompanying drawings or may be learned by production or operation of the examples. The features of the present disclosure may be realized and attained by practice or use of various aspects of the methodologies, instrumentalities, and combinations set forth in the detailed examples discussed below.

### BRIEF DESCRIPTION OF THE DRAWINGS

The present disclosure is further described in terms of exemplary embodiments.

These exemplary embodiments are described in detail with reference to the drawings. It should be noted that the drawings are not to scale. These embodiments are non-limiting exemplary embodiments, in which like reference numerals represent similar structures throughout the several views of the drawings, and wherein:

FIG. 1 is a graph illustrating a method of epitope binning;

FIG. 2A is a graph illustrating ELISA binding results of antibodies phA290, phA108, hu1H2-03, hu1H2-13, hu14G1-03, and Sirtratumab on human SLITRK6;

FIG. 2B is a graph illustrating ELISA binding results of antibodies phA290, phA108, hu1H2-03, hu1H2-13, hu14G1-03, and Sirtratumab on cynomolgus monkey SLITRK6;

FIG. 3 is a graph illustrating copy number of SLITRK6 on different SLITRK6-expressing cell lines;

FIG. 4A is a graph illustrating binding activity of antibodies to 293F-huSLITRK6-C9 cells;

FIG. 4B is a graph illustrating binding activity of antibodies to 293F cells (negative cells);

FIG. 5A is a graph illustrating binding activity of antibodies to SLITRK6-expressing tumor cell lines SW780, RT4, RT112/84, CHP-212, and SCC-4;

FIG. 5B is a graph illustrating binding activity of antibodies to SLITRK6-expressing tumor cell line SW780;

FIG. 5C is a graph illustrating binding activity of antibodies to SLITRK6-expressing tumor cell line RT4;

FIG. 5D is a graph illustrating binding activity of antibodies to SLITRK6-expressing tumor cell line RT112/84;

FIG. 5E is a graph illustrating binding activity of antibodies to SLITRK6-expressing tumor cell line CHP-212;

FIG. 6A is a graph illustrating binding activity of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) to SLITRK6-expressing cell line SW780;

FIG. 6B is a graph illustrating binding activity of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) to SLITRK6-expressing cell line RT4;

FIG. 6C is a graph illustrating binding activity of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) to SLITRK6-expressing cell line 293F-huSLITRK6-C9;

FIG. 6D is a graph illustrating binding activity of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) to SLITRK6-expressing cell line CHP-212;

FIG. 7A is a graph illustrating internalization rates of antibodies (phA290, phA108, and Sirtratumab) and ADCs (phA290-LD038 (8), phA108-LD038 (8), and Sirtratumab-LD038 (8)) in 293F-SLITRK6-C9 cells;

FIG. 7B is a graph illustrating internalization rates of antibodies (hu1H2-03, hu1H2-13, and hu14G1-03) and ADCs (hu1H2-03-LD038 (8), hu1H2-13-LD038 (8), and hu14G1-03-LD038 (8)) in 293F-SLITRK6-C9 cells;

FIG. 7C is a graph illustrating internalization rates of antibodies (hu1H2-03, hu1H2-13, and hu14G1-03) and ADCs (hu1H2-03-LD038 (8), hu1H2-13-LD038 (8), and hu14G1-03-LD038 (8)) in RT4 cells;

FIG. 7D is a graph illustrating internalization rates of antibodies (hu1H2-03, hu1H2-13, and hu14G1-03) and ADCs (hu1H2-03-LD038 (8), hu1H2-13-LD038 (8), and hu14G1-03-LD038 (8)) in RT4 cells;

FIG. 8A is a graph illustrating cytotoxicity of ADCs with linker-drug LD038 on 293F-SLITRK6-C9 cells;

FIG. 8B is a graph illustrating cytotoxicity of ADCs with linker-drug LD038 on 293F cells (negative cells);

FIG. 8C is a graph illustrating cytotoxicity of ADCs with linker-drug LD038 on RT4 cells;

FIG. 8D is a graph illustrating cytotoxicity of ADCs with linker-drug LD038 on CHP-212 cells;

FIG. 9A is a graph illustrating in vivo efficacy of single dose of SLITRK6-targeting ADCs phA290-LALA-LD038 (8), AGS15E, b12-WT-LD038 on CHP-212;

FIG. 9B is a graph illustrating in vivo efficacy of single dose of SLITRK6-targeting ADCs phA108-WT-LD038 (8), hu14G1-03-WT-LD038 (8), hu1H2-03-WT-LD038 (8), and AGS15E on CHP-212;

FIG. 9C is a graph illustrating in vivo efficacy of multiple dose of SLITRK6-targeting ADCs phA108-WT-LD038 (8), hu14G1-03-WT-LD038 (8), hu1H2-03-WT-LD038 (8), and AGS15E on CHP-212;

FIG. 9D is a graph illustrating in vivo efficacy of single dose and multiple dose of SLITRK6-targeting ADCs phA108-WT-LD038 (8), hu14G1-03-WT-LD038 (8), hu1H2-03-WT-LD038 (8), and AGS15E on SW780; and

FIG. 9E is a graph illustrating in vivo efficacy of multiple dose of SLITRK6-targeting ADCs phA290-WT-LD038 (8), phA108-WT-LD038 (8), hu14G1-03-WT-LD038 (8), hu1H2-03-WT-LD038 (8), hu1H2-13-WT-LD038 (8), AGS15E, and b12-WT-LD038 (8) on RT4.

FIG. 10A is a graph illustrating binding activity of antibodies (hu1H2-03 and Sirtratumab) and their respective ADCs (hu1H2-03-LD038 and AGS15E) to SLITRK6-expressing tumor cell line RT4;

FIG. 10B is a graph illustrating binding activity of antibodies (hu1H2-03 and Sirtratumab) and their respective ADCs (hu1H2-03-LD038 and AGS15E) to SW780 cells;

FIG. 10C is a graph illustrating binding activity of antibodies (hu1H2-03 and Sirtratumab) and their respective ADCs (hu1H2-03-LD038 and AGS15E) to RT112/84 cells;

FIG. 10D is a graph illustrating binding activity of antibodies (hu1H2-03 and Sirtratumab) and their respective ADCs (hu1H2-03-LD038 and AGS15E) to CHP-212 cells;

FIG. 10E is a graph illustrating binding activity of antibodies (hu1H2-03 and Sirtratumab) and their respective ADCs (hu1H2-03-LD038 and AGS15E) to KYSE-30 cells;

FIG. 10F is a graph illustrating binding activity of antibodies (hu1H2-03 and Sirtratumab) and their respective ADCs (hu1H2-03-LD038 and AGS15E) to KYSE-410 cells;

FIG. 10G is a graph illustrating binding activity of antibodies (hu1H2-03 and Sirtratumab) and their respective ADCs (hu1H2-03-LD038 and AGS15E) to 293F-huSLITRK6 cells;

FIG. 10H is a graph illustrating binding activity of antibodies (hu1H2-03 and Sirtratumab) and their respective ADCs (hu1H2-03-LD038 and AGS15E) to negative cells.

FIG. 11A is a graph illustrating plasma PK of hu1H2-03-LD038 (8) or hu1H2-03 in rats (n=3/group);

FIG. 11B is a graph illustrating plasma PK of hu1H2-03-LD038 (8) in monkeys (n=2);

FIG. 12 is a graph illustrating hydrophobicity interaction chromatograph of hu1H2-03-LD038 (8), hu1H2-03-LD343 (8), AGS15E, and their parent mAbs (hu1H2-03, sirtratumab);

FIG. 13A is a graph illustrating binding activity of antibodies (hu1H2-03 and Sirtratumab) and their respective ADCs (hu1H2-03-LD343 and AGS15E) to SW780 cells;

FIG. 13B is a graph illustrating binding activity of antibodies (hu1H2-03 and Sirtratumab) and their respective ADCs (hu1H2-03-LD343 and AGS15E) to RT4 cells;

FIG. 14A is a graph illustrating internalization rates of antibodies (hu1H2-03 and Sirtratumab) and their respective ADCs (hu1H2-03-LD038 and AGS15E) in SW780 cells;

FIG. 14B is a graph illustrating internalization rates of antibodies (hu1H2-03 and Sirtratumab) and their respective ADCs (hu1H2-03-LD038 and AGS15E) in RT4 cells;

FIG. 14C is a graph illustrating internalization rates of antibodies (hu1H2-03 and Sirtratumab) and their respective ADCs (hu1H2-03-LD343 and AGS15E) in RT4 cells;

FIG. 15A is a graph illustrating cytotoxicity of hu1H2-03-LD038 and AGS15E on 293F-SLITRK6 cells;

FIG. 15B is a graph illustrating cytotoxicity of hu1H2-03-LD038 and AGS15E on 293F cells;

FIG. 15C is a graph illustrating cytotoxicity of hu1H2-03-LD038 and AGS15E on SW780 cells;

FIG. 15D is a graph illustrating cytotoxicity of hu1H2-03-LD038 and AGS15E on RT4 cells;

FIG. 15E is a graph illustrating cytotoxicity of hu1H2-03-LD038 and AGS15E on RT112/84 cells;

FIG. 15F is a graph illustrating cytotoxicity of hu1H2-03-LD038 and AGS15E on CHP-212 cells;

FIG. 15G is a graph illustrating cytotoxicity of hu1H2-03-LD038 and AGS15E on KYSE-410 cells;

FIG. 15H is a graph illustrating cytotoxicity of hu1H2-03-LD038 and AGS15E on KYSE-30 cells;

FIG. 15I is a graph illustrating cytotoxicity of hu1H2-03-LD343 and AGS15E on SW780 cells;

FIG. 15J is a graph illustrating cytotoxicity of hu1H2-03-LD343 and AGS15E on RT4 cells;

FIG. 16A is a graph illustrating in vivo efficacy of single dose of hu1H2-03-LD038 analog, hu1H2-03-LD038, and AGS15E on SW780 cells;

FIG. 16B is a graph illustrating in vivo efficacy of single dose of hu1H2-03-LALA-LD038 and AGS15E on RT4 cells;

FIG. 16C is a graph illustrating in vivo efficacy of single dose of hu1H2-03-WT-LD038 and AGS15E on CHP-212 cells;

FIG. 16D is a graph illustrating in vivo efficacy of single dose of hu1H2-03-LD038 and AGS15E on KYSE-410 cells;

FIG. 16E is a graph illustrating in vivo efficacy of single dose of hu1H2-03-LD038 and AGS15E on KYSE-30 cells;

FIG. 16F is a graph illustrating in vivo efficacy of single dose of hu1H2-03-LD343 and AGS15E on SW780 cells;

FIG. 16G is a graph illustrating in vivo efficacy of single dose of hu1H2-03-LD343 and AGS15E on RT4 cells; and

FIG. 17 is a graph illustrating in vivo efficacy of single dose of hu1H2-03-LD038, hu1H2-03-LD343, and AGS15E on bladder cancer cells in PDX models.

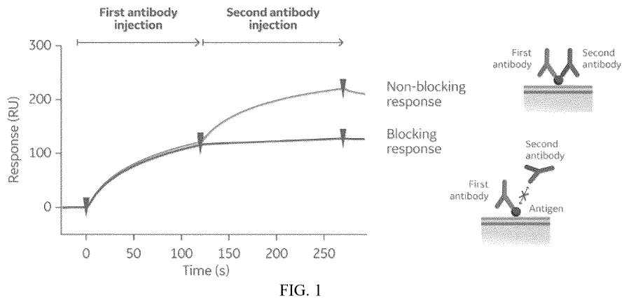

### DETAILED DESCRIPTION

The following description is presented to enable any person skilled in the art to make and use the present disclosure and is provided in the context of a particular application and its requirements. Various modifications to the disclosed embodiments will be readily apparent to those skilled in the art, and the general principles defined herein may be applied to other embodiments and applications without departing from the spirit and scope of the present disclosure. Thus, the present disclosure is not limited to the embodiments shown but is to be accorded the widest scope consistent with the claims.

The terminology used herein is to describe particular example embodiments only and is not intended to be limiting. As used herein, the singular forms “a,” “an,” and “the” may be intended to include the plural forms as well, unless the context clearly indicates otherwise. It will be further understood that the terms “comprises,” “comprising,” “includes,” and/or “including” when used in this specification, specify the presence of stated features, integers, steps, operations, elements, and/or components, but do not preclude the presence or addition of one or more other features, integers, steps, operations, elements, components, and/or groups thereof.

These and other features, and characteristics of the present disclosure, as well as the methods of operation and functions of the related elements of structure and the combination of parts and economies of manufacture, may become more apparent upon consideration of the following description with reference to the accompanying drawing(s), all of which form a part of this specification. It is to be expressly understood, however, that the drawing(s) is for the purpose of illustration and description only and are not intended to limit the scope of the present disclosure. It is understood that the drawings are not to scale.

### Definitions

For convenience, certain terms in the specification, examples and claims are defined here. Unless stated otherwise, or implicit from context, the following terms and phrases have the meanings provided below. The definitions are provided to aid in describing particular embodiments, and are not intended to limit the claimed invention, because the scope of the invention is limited only by the claims. Unless otherwise defined, all technical and scientific terms used herein have the same meaning as commonly understood by one of ordinary skill in the art to which this invention belongs.

As used herein and unless otherwise indicated, the terms “a” and “an” are taken to mean “one”, “at least one” or “one or more”. Unless otherwise required by context, singular terms used herein shall include pluralities and plural terms shall include the singular.

Unless the context clearly requires otherwise, throughout the description and the claims, the words “comprise”, “comprising”, and the like are to be construed in an inclusive sense as opposed to an exclusive or exhaustive sense; that is to say, in the sense of “including, but not limited to”.

The terms “decreased,” “reduce,” “reduced”, “reduction”, “decrease,” and “inhibit” are all used herein generally to mean a decrease by a statistically significant amount relative to a reference.

The terms “increased”, “increase” or “enhance” or “activate” are all used herein to generally mean an increase by a statically significant amount relative to a reference.

As used herein, the terms “protein” and “polypeptide” are used interchangeably herein to designate a series of amino acid residues each connected to each other by peptide bonds between the alpha-amino and carboxyl groups of adjacent residues. The terms “protein” and “polypeptide” also refer to a polymer of amino acids, including modified amino acids (e.g., phosphorylated, glycated, glycosylated, etc.) and amino acid analogs, regardless of its size or function. “Protein” and “polypeptide” are often used in reference to relatively large polypeptides, whereas the term “peptide” is often used in reference to small polypeptides, but usage of these terms in the art overlaps. The terms “protein” and “polypeptide” are used interchangeably herein when referring to an encoded gene product and fragments thereof. Thus, exemplary polypeptides or proteins include gene products, naturally occurring proteins, homologs, orthologs, paralogs, fragments and other equivalents, variants, fragments, and analogs of the foregoing.

SLITRK6 (protein tyrosine kinase-like 7) is a member of the receptor protein tyrosine kinase family and is also known as colon carcinoma kinase 4 (CCK4). In humans, this protein is encoded by the SLITRK6 gene. SLITRK6 is overexpressed on the surface of many types of cancer, including but not limited to solid tumors. Such solid tumors include but are not limited to breast cancer (BC), lung cancer (LC), esophageal cancer (EsC), gastric cancer (GC), bladder cancer (BLC), endometrial cancer (EC), ovarian cancer (OVC), head and neck cancer (HNC) as well as hematological malignancies. Human SLITRK6 polypeptides include, but are not limited to, those having the amino acid sequences set forth in UniProt identifiers Q13308, and those having the amino acid sequences set forth in Genbank such as but not limited to GenBank accession numbers NP_002812.2, NP_690619.1, NP_690620.1, NP_690621.1, NP_001257327.1, XP_011513067.1, XP_011513068.1, XP_047275113.1, XP_054212039.1, XP_054212040.1, and XP_054212041.1, which are incorporated by reference herein. Although lacking detectable catalytic tyrosine kinase activity, SLITRK6 overexpression is intimately involved in Wnt signaling and promotes cancer cell stemness, survival and tumor progression (See e.g., Atasaven et al., 2013; Cui et al., 2021; Gärtner, 2014; Chen et al., 2014; Jiang et al., 2020; Shin et al., 2013; Liu et al., 2017; Lin et al., 2012; Özçelik et al., 2020; Xiang et al., 2022; Wang et al., 2014; Prebet et al., 2010; Jiang et al., 2012; Damelin et al., 2017). SLITRK6 expression in normal tissues is generally low, although some levels of protein expression have been reported in the digestive tract, such as urinary bladder, kidney, mammary gland, lung, ovary, uterus, and dendritic cells (Damelin et al., 2017). SLITRK6 is thus a promising target for novel anti-cancer therapy.

As used herein, an “epitope” refers to the amino acids conventionally bound by an immunoglobulin VH/VL pair, such as the antibodies, antigen binding portions thereof and other binding agents described herein. An epitope can be formed on a polypeptide from contiguous amino acids or noncontiguous amino acids juxtaposed by tertiary folding of a protein. Epitopes formed from contiguous amino acids are typically retained on exposure to denaturing solvents, whereas epitopes formed by tertiary folding are typically lost on treatment with denaturing solvents. An epitope typically includes at least 3, and more usually, at least 5, about 9, or about 8-10 amino acids in a unique spatial conformation. An epitope defines the minimum binding site for an antibody, antigen binding portions thereof and other binding agent, and thus represents the target of specificity of an antibody, antigen binding portion thereof or other immunoglobulin-based binding agent. In the case of a single domain antibody, an epitope represents the unit of structure bound by a variable domain in isolation.

As used herein, “specifically binds” refers to the ability of a binding agent (e.g., an antibody or antigen binding portion thereof) described herein to bind to a target, such as human SLITRK6, with a KD of 10-5 M (10000 nM) or less, e.g., 10-6 M, 10-7 M, 10-8 M, 10-9 M, 10-10 M, 10-11 M, 10-12 M, or less. Specific binding can be influenced by, for example, the affinity and avidity of the antibody, antigen binding portion or other binding agent and the concentration of target polypeptide. The person of ordinary skill in the art can determine appropriate conditions under which the antibodies, antigen binding portions and other binding agents described herein selectively bind to SLITRK6 using any suitable methods, such as titration of an antibody or other binding agent in a suitable cell binding assay. A binding agent specifically bound to SLITRK6 is not displaced by a non-similar competitor. In certain embodiments, a SLITRK6 antibody or antigen-binding portion thereof or other binding agent is said to specifically bind to SLITRK6 when it preferentially recognizes its target antigen, SLITRK6, in a complex mixture of proteins and/or macromolecules.

In some embodiments, a SLITRK6 antibody or antigen-binding portion thereof or other binding agent as described herein specifically binds to a SLITRK6 polypeptide with a dissociation constant (KD) of 10-5 M (10000 nM) or less, e.g., 10⁻⁶ M, 10⁻⁷ M, 10⁻⁸ M, 10⁻⁹ M, 10⁻¹⁰ M, 10⁻¹¹ M, 10⁻¹² M, or less. In some embodiments, a SLITRK6 antibody or antigen-binding portion thereof or other binding agent as described herein specifically binds to a SLITRK6 polypeptide with a dissociation constant (KD) of from about 10⁻⁵ M to 10⁻⁶ M. In some embodiments, a SLITRK6 antibody or antigen-binding portion thereof or other binding agent as described herein specifically binds to a SLITRK6 polypeptide with a dissociation constant (KD) of from about 10⁻⁷ M to 10⁻⁸ M. In some embodiments, a SLITRK6 antibody or antigen-binding portion thereof or other binding agent as described herein specifically binds to a SLITRK6 polypeptide with a dissociation constant (KD) of from about 10⁻⁸ M to 10⁻⁹ M. In some embodiments, a SLITRK6 antibody or antigen-binding portion thereof or other binding agent as described herein specifically binds to a SLITRK6 polypeptide with a dissociation constant (KD) of from about 10⁻⁹ M to 10⁻¹⁰ M. In some embodiments, a SLITRK6 antibody or antigen-binding portion thereof or other binding agent as described herein specifically binds to a SLITRK6 polypeptide with a dissociation constant (KD) of from about 10⁻¹⁰ M to 10⁻¹¹ M. In some embodiments, a SLITRK6 antibody or antigen-binding portion thereof or other binding agent as described herein specifically binds to a SLITRK6 polypeptide with a dissociation constant (KD) of from about 10⁻¹¹ M to 10⁻¹² M. In some embodiments, a SLITRK6 antibody or antigen-binding portion thereof or other binding agent as described herein specifically binds to a SLITRK6 polypeptide with a dissociation constant (KD) of from about 10⁻¹² M to 10⁻¹³ M. In some embodiments, a SLITRK6 antibody or antigen-binding portion thereof or other binding agent as described herein specifically binds to a SLITRK6 polypeptide with a dissociation constant (KD) of less than 10⁻⁹ M.

Unless otherwise indicated, the term “alkyl” by itself or as part of another term refers to a substituted or unsubstituted straight chain or branched, saturated hydrocarbon having the indicated number of carbon atoms (e.g., “—C1-C5 alkyl”, “—C1-C8 alkyl” or “—C1-C10” alkyl refer to an alkyl group having from 1 to 5, 1 to 8, or 1 to 10 carbon atoms, respectively). Examples include methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), and 3,3-dimethyl-2-butyl (—CH(CH₃)C(CH₃)₃.

Unless otherwise indicated, “alkenyl” by itself or as part of another term refers to a C2-C8 substituted or unsubstituted straight chain or branched, hydrocarbon with at least one site of unsaturation (i.e., a carbon-carbon, sp2 double bond). Examples include, but are not limited to: ethylene or vinyl (—CH═CH₂), allyl (—CH₂CH═CH₂), cyclopentenyl (—C₅H₇), and 5-hexenyl (—CH₂CH₂CH₂CH₂CH═CH₂).

Unless otherwise indicated, “alkynyl” by itself or as part of another term refers to a refers to C2-C8, substituted or unsubstituted straight chain or branched, hydrocarbon with at least one site of unsaturation (i.e., a carbon-carbon, sp triple bond. Examples include, but are not limited to: acetylenic and propargyl.

Unless other indicated, “alkylene” refers to a saturated, branched or straight chain or hydrocarbon radical of 1-8 carbon atoms, and having two monovalent radical centers derived by the removal of two hydrogen atoms from the same or two different carbon atoms of a parent alkane. Typical alkylene radicals include, but are not limited to: methylene (—CH₂—), 1,2-ethyl (—CH₂CH₂—), 1,3-propyl (—CH₂CH₂CH₂—), 1,4-butyl (—CH₂CH₂CH₂CH₂—), and the like.

Unless otherwise indicated, “alkenylene” refers to an unsaturated, branched or straight chain hydrocarbon radical of 2-8 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

Unless otherwise indicated, “alkynylene” refers to an unsaturated, branched or straight chain or cyclic hydrocarbon radical of 2-8 carbon atoms, and having two monovalent radical centers derived by the removal of two hydrogen atoms from the same or two different carbon atoms of a parent alkyne. Typical alkynylene radicals include, but are not limited to: acetylene, propargyl, and 4-pentynyl.

Unless otherwise indicated, the term “heteroalkyl,” by itself or in combination with another term, refers to a substituted or unsubstituted stable straight or branched chain hydrocarbon, or combinations thereof, saturated and from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of the heteroalkyl group (i.e., as part of the main chain) or at the position at which the alkyl group is attached to the remainder of the molecule. The heteroatom Si may be placed at any position of the heteroalkyl group, including the position at which the alkyl group is attached to the remainder of the molecule. Examples of heteroalkyl include the following: —CH₂CH₂OCH₃, —CH₂CH₂NHCH₃, —CH₂CH₂N(CH₃)CH₃, —CH₂SCH₂CH₃, CH₂CH₂S(O)CH₃, —CH₂CH₂S(O)₂CH₃, and —Si(CH₃)₃. Up to two heteroatoms may be consecutive, such as, for example, —CH₂NHOCH₃ and CH₂OSi(CH₃)₃. In some embodiments, a C1 to C4 heteroalkyl has 1 to 4 carbon atoms and 1 or 2 heteroatoms and a C1 to C3 heteroalkyl has 1 to 3 carbon atoms and 1 or 2 heteroatoms.

Unless otherwise indicated, the terms “heteroalkenyl” and “heteroalkynyl” by themselves or in combination with another term, refers to a substituted or unsubstituted stable straight or branched chain alkenyl or alkynyl having from one to ten, preferably one to three, heteroatoms selected from the group consisting of O, N, Si and S, and wherein the nitrogen and sulfur atoms may optionally be oxidized and the nitrogen heteroatom may optionally be quaternized. The heteroatom(s) O, N and S may be placed at any interior position of a heteroalkenyl or heteroalkynyl group (i.e., as part of the main chain) or at the position at which the alkyl group is attached to the remainder of the molecule. The heteroatom Si may be placed at any position of a heteroalkenyl or heteroalkynyl group, including the position at which the alkyl group is attached to the remainder of the molecule.

Unless otherwise indicated, the term “heteroalkylene” by itself or as part of another substituent refers to a substituted or unsubstituted divalent group derived from a heteroalkyl (as discussed above), as exemplified by —CH2CH2SCH2CH2- and —CH2SCH2CH2NHCH2-. In some embodiments, a C1 to C4 heteroalkylene has 1 to 4 carbon atoms and 1 or 2 heteroatoms and a C1 to C3 heteroalkylene has 1 to 3 carbon atoms and 1 or 2 heteroatoms. For heteroalkylene groups, heteroatoms can also occupy either or both of the chain termini. Still further, for alkylene and heteroalkylene linking groups, no orientation of the linking group is implied.

Unless otherwise indicated, the terms “heteroalkenylene” and “heteroalkynylene” by themselves or as part of another substituent refers to a substituted or unsubstituted divalent group derived from an heteroalkenyl or heteroalkynyl (as discussed above). In some embodiments, a C2 to C4 heteroalkenylene or heteroalkynylene has 1 to 4 carbon atoms. For heteroalkenylene and heteroalkynylene groups, heteroatoms can also occupy either or both of the chain termini. Still further, for alkylene and heteroalkenylene and heteroalkynylene linking groups, no orientation of the linking group is implied.

Unless otherwise indicated, a “C3-C8 carbocycle,” by itself or as part of another term, refers to a substituted or unsubstituted 3-, 4-, 5-, 6-, 7- or 8-membered monovalent, substituted or unsubstituted, saturated or unsaturated non-aromatic monocyclic or bicyclic carbocyclic ring derived by the removal of one hydrogen atom from a ring atom of a parent ring system. Representative —C3-C8 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl.

Unless otherwise indicated, a “C3-C8 carbocyclo”, by itself or as part of another term, refers to a substituted or unsubstituted C3-C8 carbocycle group defined above wherein another of the carbocycle groups' hydrogen atoms is replaced with a bond (i.e., it is divalent).

Unless otherwise indicated, a “C3-C10 carbocycle,” by itself or as part of another term, refers to a substituted or unsubstituted 3-, 4-, 5-, 6-, 7-, 8-, 9- or 10-membered monovalent, substituted or unsubstituted, saturated or unsaturated non-aromatic monocyclic, bicyclic or tricyclic carbocyclic ring derived by the removal of one hydrogen atom from a ring atom of a parent ring system. Representative —C3-C10 carbocycles include, but are not limited to, cyclopropyl, cyclobutyl, cyclopentyl, cyclopentadienyl, cyclohexyl, cyclohexenyl, 1,3-cyclohexadienyl, 1,4-cyclohexadienyl, cycloheptyl, 1,3-cycloheptadienyl, 1,3,5-cycloheptatrienyl, cyclooctyl, and cyclooctadienyl. —C3-C10 carbocycles can further include fused cyclooctyne carbocycles, such as the fused cyclooctyne compounds disclosed in International Publication Number WO2011/136645 (the disclosure of which is incorporated by reference herein), including BCN (bicyclo[6.1.0]nonyne) and DBCO (Dibenzocyclooctyne).

Unless otherwise indicated, a “C3-C8 heterocycle,” by itself or as part of another term, refers to a substituted or unsubstituted monovalent substituted or unsubstituted aromatic or non-aromatic monocyclic or bicyclic ring system having from 3 to 8 carbon atoms (also referred to as ring members) and one to four heteroatom ring members independently selected from N, O, P or S, and derived by removal of one hydrogen atom from a ring atom of a parent ring system. One or more N, C or S atoms in the heterocycle can be oxidized. The ring that includes the heteroatom can be aromatic or nonaromatic. Unless otherwise noted, the heterocycle is attached to its pendant group at any heteroatom or carbon atom that results in a stable structure. Representative examples of a C3-C8 heterocycle include, but are not limited to, pyrrolidinyl, azetidinyl, piperidinyl, morpholinyl, tetrahydrofuranyl, tetrahydropyranyl, benzofuranyl, benzothiophene, indolyl, benzopyrazolyl, pyrrolyl, thiophenyl (thiophene), furanyl, thiazolyl, imidazolyl, pyrazolyl, pyrimidinyl, pyridinyl, pyrazinyl, pyridazinyl, isothiazolyl, and isoxazolyl. Unless otherwise indicate, the term “heterocarbocycle” is synonymous with the terms “heterocycle” or “heterocyclo” as described herein.

Unless otherwise indicated, “C3-C8 heterocyclo”, by itself or as part of another term, refers to a substituted or unsubstituted C3-C8 heterocycle group defined above wherein one of the heterocycle group's hydrogen atoms is replaced with a bond (i.e., it is divalent).

Unless otherwise indicated, “aryl” by itself or as part of another term, means a substituted or unsubstituted monovalent carbocyclic aromatic hydrocarbon radical of 6-20 carbon (preferably 6-14 carbon) atoms derived by the removal of one hydrogen atom from a single carbon atom of a parent aromatic ring system. Some aryl groups are represented in the exemplary structures as “Ar”. Typical aryl groups include, but are not limited to, radicals derived from benzene, substituted benzene, naphthalene, anthracene, biphenyl, and the like. An exemplary aryl group is a phenyl group.

Unless otherwise indicated, an “arylene” by itself or as part of another term, is an unsubstituted or substituted aryl group as defined above wherein one of the aryl group's hydrogen atoms is replaced with a bond (i.e., it is divalent) and can be in the ortho, meta, or para orientations.

Unless otherwise indicated, “heteroaryl” and “heterocycle” refer to a ring system in which one or more ring atoms is a heteroatom, e.g., nitrogen, oxygen, and sulfur. A heterocycle radical comprises 1 to 20 carbon atoms and 1 to 3 heteroatoms selected from N, O, P, and S. A heterocycle may be a monocycle having 3 to 7 ring members (2 to 6 carbon atoms and 1 to 3 heteroatoms selected from N, O, P, and S) or a bicycle having 7 to 10 ring members (4 to 9 carbon atoms and 1 to 3 heteroatoms selected from N, O, P, and S), for example: a bicyclo [4,5], [5,5], [5,6], or [6,6] system.

Unless otherwise indicated, an “heteroarylene” by itself or as part of another term, is an unsubstituted or substituted heteroaryl group as defined above wherein one of the heteroaryl group's hydrogen atoms is replaced with a bond (i.e., it is divalent).

Unless otherwise indicated, “carboxyl” refers to COOH or COO-M+, where M+ is a cation.

Unless otherwise indicated, “oxo” refers to (C═O).

Unless otherwise indicated, “substituted alkyl” and “substituted aryl” mean alkyl and aryl, respectively, in which one or more hydrogen atoms are each independently replaced with a substituent. Typical substituents include, but are not limited to, —X, —R10, —O—, —OR10, —SR10, —S—, —NR102, —NR103, ═NR10, —CX3, —CN, —OCN, —SCN, —N═C═O, —NCS, —NO, —NO2, ═N2, —N3, —NR10C(═O)R10, —C(═O)R10, —C(═O)NR102, —SO3-, —SO3H, —S(═O)2R10, —OS(═O)2OR10, —S(═O)2NR10, —S(═O)R10, —OP(═O)(OR10)2, —P(═O)(OR10)2, —PO-3, —PO3H2, —AsO2H2, —C(═O)R10, —C(═O)X, —C(═S)R10, —CO2R10, —CO2-, —C(═S)OR10, C(═O)SR10, C(═S)SR10, C(═O)NR102, C(═S)NR102, or C(═NR10)NR102, where each X is independently a halogen: —F, —C, —Br, or —I; and each R10 is independently —H, —C1-C20 alkyl, —C6-C20 aryl, —C3-C14 heterocycle, a protecting group or a prodrug moiety. Typical substitutents also include (═O). Alkylene, carbocycle, carbocyclo, arylene, heteroalkyl, heteroalkylene, heterocycle, and heterocyclo groups as described above may also be similarly substituted.

Unless otherwise indicated, “polyhydroxyl group” refers to an alkyl, alkylene, carbocycle or carbocyclo group including two or more, or three or more, substitutions of hydroxyl groups for hydrogen on carbon atoms of the carbon chain. In some embodiments, a polyhydroxyl group comprises at least three hydroxyl groups. In some embodiments, a polyhydroxyl group comprises carbon atoms containing only one hydroxyl group per carbon atom. A polyhydroxyl group may contain one or more carbon atoms that are not substituted with hydroxyl. A polyhydroxyl group may have each carbon atom substituted with a hydroxyl group. Examples of polyhydroxyl group includes linear (acyclic) or cyclic forms of monosaccharides such as C6 or C5 sugars, such as glucose, ribose, galactose, mannose, arabinose, 2-deoxyglucose, glyceraldehyde, erythrose, threose, xylose, lyxose, allose, altrose, gulose, idose, talose, aldose, and ketose, sugar acids such as gluconic acid, aldonic acid, uronic acid or ulosonic acid, and an amino sugars, such as glucosamine, N-acetyl glucosamine, galactosamine, and N-acetyl galactosamine. In some embodiments, polyhydroxyl group includes linear or cyclic forms of disaccharides and polysaccharides.

Unless otherwise indicated by context, “optionally substituted” refers to an alkyl, alkenyl, alkynyl, alkylaryl, arylalkyl heterocycle, aryl, heteroaryl, alkylheteroaryl, heteroarylalkyl, or other substituent, moiety or group as defined or disclosed herein wherein hydrogen atom(s) of that substituent, moiety or group has been optionally replaced with different moiety(ies) or group(s), or wherein an alicyclic carbon chain that comprise one of those substituents, moiety or group is interrupted by replacing carbon atom(s) of that chain with different moiety(ies) or group(s). In some aspects an alkene function group replaces two contiguous sp3 carbon atoms of an alkyl substituent, provided that the radical carbon of the alkyl moiety is not replaced, so that the optionally substituted alkyl is an unsaturated alkyl substituent.

Optional substituent replacing hydrogen(s) in any of the foregoing substituents, moieties or groups is independently selected from the group consisting of aryl, heteroaryl, hydroxyl, alkoxy, aryloxy, cyano, halogen, nitro, fluoroalkoxy, and amino, including mono-, di- and tri-substituted amino groups, and the protected derivatives thereof, or is selected from the group consisting of —X, —OR′, —SR′, —NH2, —N(R′)(R″), —N(R″)3, ═NR, —CX3, —CN, —NO2, —NR′C(═O)H, —NR′C(═O)R, —NR′C(═O)R″, —C(═O)R′, —C(═O)NH2, —C(═O)N(R′)R″, —S(═O)2R″, —S(═O)2NH2, —S(═O)2N(R′)R″, —S(═O)2NH2, —S(═O)2N(R′)R″, —S(═O)2OR′, —S(═O)R″, —OP(═O)(OR′)(OR″), —OP(OH)3, —P(═O)(OR′)(OR″), —PO3H2, —C(═O)R′, —C(═S)R″, —CO2R′, —C(═S)OR″, —C(═O)SR′, —C(═S)SR′, —C(═S)NH2, —C(═S)N(R′)(R″)2, —C(═NR′)NH2, —C(═NR′)N(R′)R″, and salts thereof, wherein each X is independently selected from the group consisting of a halogen: —F, —Cl, —Br, and —I; and wherein each R″ is independently selected from the group consisting of C1-C20 alkyl, C2-C20 alkenyl, C2-C20 alkynyl, C6-C24 aryl, C3-C24 heterocyclyl (including C5-C24 heteroaryl), a protecting group, and a prodrug moiety or two of R″ together with the heteroatom to which they are attached defines a heterocyclyl; and R′ is hydrogen or R″, wherein R″ is selected from the group consisting of C1-C20 alkyl, C6-C24 aryl, C3-C24 heterocyclyl (including C5-C24 heteroaryl), and a protecting group.

Typically, optional substituents are selected from the group consisting of —X, —OH, —OR″, —SH, —SR″, —NH2, —NH(R″), —NR′(R″)2, —N(R″)3, ═NH, ═NR″, —CX3, —CN, —NO2, —NR′C(═O)H, NR′C(═O)R″, —CO2H, —C(═O)H, —C(═O)R″, —C(═O)NH2, —C(═O)NR′R″— —S(═O)2R″, —S(═O)2NH2, —S(═O)2N(R′)R″, —S(═O)2NH2, —S(═O)2N(R′)(R″), —S(═O)2OR′, —S(═O)R″, —C(═S)R″, —C(═S)NH2, —C(═S)N(R′)R″, —C(═NR′)N(R″)2, and salts thereof, wherein each X is independently selected from the group consisting of —F and —Cl, R″ is typically selected from the group consisting of C1-C6 alkyl, C6-C10 aryl, C3-C10 heterocyclyl (including C5-C10 heteroaryl), and a protecting group; and R′ independently is hydrogen, C1-C6 alkyl, C6-C10 aryl, C3-C10 heterocyclyl (including C5-C10 heteroaryl), and a protecting group, independently selected from R″. More typically, substituents are selected from the group consisting of —X, —R″, —OH, —OR″, —NH2, —NH(R″), —N(R″)2, —N(R″)3, —CX3, —NO2, —NHC(═O)H, —NHC(═O)R″, —C(═O)NH2, —C(═O)NHR″, —C(═O)N(R″)2, —CO2H, —CO2R″, —C(═O)H, —C(═O)R″, —C(═O)NH2, —C(═O)NH(R″), —C(═O)N(R″)2, —C(═NR′)NH2, —C(═NR′)NH(R″), —C(═NR′)N(R″)2, a protecting group and salts thereof, wherein each X is —F, R″ is independently selected from the group consisting of C1-C6 alkyl, C6-C10 aryl, C5-C10 heteroaryl and a protecting group; and R′ is selected from the group consisting of hydrogen, C1-C6 alkyl and a protecting group, independently selected from R″.

The compounds of the invention, or their pharmaceutically acceptable salts may contain one or more asymmetric centers and may thus give rise to enantiomers, diastereomers, and other stereoisomeric forms that are defined, in terms of absolute stereochemistry, as (R) or (S) or, as (D) or (L) for amino acids. The present invention is meant to include all such possible isomers, as well as their racemic and optically pure forms. Optically active (+) and ( ), (R) and (S), or (D) and (L) isomers may be prepared using chiral synthons or chiral reagents, or resolved using conventional techniques, for example, chromatography and fractional crystallization. Conventional techniques for the preparation/isolation of individual enantiomers include chiral synthesis from a suitable optically pure precursor or resolution of the racemate (or the racemate of a salt or derivative) using, for example, chiral high pressure liquid chromatography (HPLC). When the compounds described herein contain olefinic double bonds or other centres of geometric asymmetry, and unless specified otherwise, it is intended that the compounds include both E and Z geometric isomers. Likewise, all tautomeric forms are also intended to be included.

A “stereoisomer” refers to a compound made up of the same atoms bonded by the same bonds but having different three-dimensional structures, which are not interchangeable. The present invention contemplates various stereoisomers and mixtures thereof and includes “enantiomers”, which refers to two stereoisomers whose molecules are nonsuperimposeable mirror images of one another. The present invention also includes “diastereomers”, which refers to two or more stereoisomers of a compound that have different configurations at one or more of the equivalent stereocenters and are not mirror images of each other.

Although structures shown throughout the specification are depicted with specific stereocenters, the specification should be read to include variations in those stereocenters. For example, the structure of exatecan may be shown in the (S,S) configuration, but the (R,S) diastereomer of exatecan is also envisioned as being found in a separate embodiment of a conjugate as described herein.

Unless otherwise indicated, the term “drug unit” or drug refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

Unless otherwise indicated, the term “polymer unit” refers to a polymeric moiety composed of repeating subunits. Examples of polymer units include polyamides and polyethers. In some embodiments, the polymer unit is selected from an optionally substituted polyamide, a substituted polyether, or combinations thereof. In further embodiments, the polymer unit is selected from
(i) an optionally substituted polyamide comprising the formula

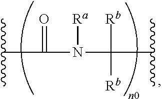

or a stereoisomer thereof, wherein each Ra is independently H or C1-6 alkyl and each Rb is independently H or C1-6 alkyl, and n0 is independently 2-26;
(ii) a substituted polyether comprising the formula

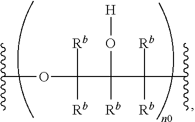

or a stereoisomer thereof, wherein each Rb is independently H or C1-6 alkyl, and n0 is independently 2-26; or
(iii) combinations thereof.

Unless otherwise indicated, the term “sugar unit” or “sugar group” refers to a carbohydrate group. Examples of sugar units include glycosides.

Unless otherwise indicated, the term “carboxyl unit” or “carboxyl group” refers to a group including a carbonyl group [—C(O)—], a carboxyl group [—CO2H], and/or a carboxylate group [—CO2M, M refers to a cationic counterion].

Unless otherwise indicated, the term “stretcher group” refers to a linking moiety that connects the SLITRK6 binding agent to the enzyme-cleavable group.

Unless otherwise indicated, the term “polyamide” refers to polymeric groups composed of repeating subunits containing amide bonds.

Unless otherwise indicated, the term “polyether” refers to polymeric groups composed to repeating subunits containing ether bonds.

Unless otherwise indicated, the term “enzyme-cleavable group” refers to a group that is cleavable by the action of a metabolic process or reaction inside a cell or in the extracellular milieu, whereby the covalent attachment between a drug unit (e.g., a cytotoxic agent) and the linker unit or portion thereof is broken, resulting in the free drug unit, or a metabolite of the linker unit-drug, which is dissociated from the remainder of the linker unit.

The phrase “pharmaceutically acceptable salt,” as used herein, refers to pharmaceutically acceptable organic or inorganic salts of a compound (e.g., a linker, drug linker, or a conjugate). The compound typically contains at least one amino group, and accordingly acid addition salts can be formed with this amino group. Exemplary salts include, but are not limited to, sulfate, citrate, acetate, oxalate, chloride, bromide, iodide, nitrate, bisulfate, phosphate, acid phosphate, isonicotinate, lactate, salicylate, acid citrate, tartrate, oleate, tannate, pantothenate, bitartrate, ascorbate, succinate, linleate, gentisinate, fumarate, gluconate, glucuronate, saccharate, formate, benzoate, glutamate, methanesulfonate, ethanesulfonate, benzenesulfonate, toluenesulfonate, and pamoate (i.e., 1,1′-methylene-bis-(2-hydroxy-3-naphthoate)) salts. A pharmaceutically acceptable salt may involve the inclusion of another molecule such as an acetate ion, a succinate ion or other counterion. The counterion may be any organic or inorganic moiety that stabilizes the charge on the parent compound. Furthermore, a pharmaceutically acceptable salt may have more than one charged atom in its structure. Instances where multiple charged atoms are part of the pharmaceutically acceptable salt can have multiple counter ions. Hence, a pharmaceutically acceptable salt can have one or more charged atoms and/or one or more counterion.

As used herein, the term “consisting essentially of” refers to those elements required for a given embodiment. The term permits the presence of elements that do not materially affect the basic and novel or functional characteristic(s) of that embodiment.

As used herein, the term “consisting of” refers to compositions, methods, and respective components thereof as described herein, which are exclusive of any element not recited in that description of the embodiment.

Other than in the examples, or where otherwise indicated, all numbers expressing quantities of ingredients or reaction conditions used herein should be understood as modified in all instances by the term “about.” The term “about” when used in connection with percentages can mean+/−1%.

The terms “statistically significant” or “significantly” refer to statistical significance and generally mean a two standard deviation (2SD) difference, above or below a reference value.

Other terms are defined herein within the description of the various aspects of the invention.

### Antibodies and Binding Agents

Provided herein are SLITRK6 binding antibodies (also referred to as SLITRK6 antibodies) and antigen binding portions thereof and other binding agents that specifically bind to human SLITRK6. Also provided herein are conjugates of the SLITRK6 antibodies and antigen binding portions and other binding agents bound to drugs, such as cytotoxic agents or immune modulatory agents (also referred to as SLITRK6 conjugates). In some embodiments, the SLITRK6 antibodies, antigen binding portions, other binding agents and/or SLITRK6 conjugates specifically bind to and reduce the number of SLITRK6+ cells in a subject. In some embodiments, the SLITRK6 antibodies, antigen binding portions, other binding agents and/or SLITRK6 conjugates specifically bind to and reduce the number of SLITRK6+ cancer cells in a subject. In some embodiments, the SLITRK6 antibodies, antigen binding portions, other binding agents and/or SLITRK6 conjugates specifically bind to and reduce the number of SLITRK6+ cells associated with a disease or condition in a subject, such as a cancer or an autoimmune disease. In some embodiments, the SLITRK6 antibodies, antigen binding portions, other binding agents and/or SLITRK6 conjugates specifically bind to and reduce the number of SLITRK6+ cells associated with a disease or condition in a subject, such as a human or an animal.

In some embodiments, the SLITRK6 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 1 and SEQ ID NO: 2, respectively; SEQ ID NO: 9 and SEQ ID NO:10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively. In some embodiments, the SLITRK6 antibodies or antigen binding portions thereof comprise a heavy chain variable (VH) region and a light chain variable (VL) region, the VH and VL regions having the amino acid sequences set forth in SEQ ID NO:1 and SEQ ID NO:2, respectively. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 9 and SEQ ID NO: 10, respectively. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 17 and SEQ ID NO: 18, respectively. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 25 and SEQ ID NO: 26, respectively. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 33 and SEQ ID NO: 34, respectively.

In some embodiments, the SLITRK6 antibodies or antigen binding portions thereof comprise a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable (VH) region and a light chain variable (VL) region, the VH and VL regions having the amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified. The phrase “wherein the CDRs of the heavy or light chain variable regions are not modified” refers to the VH and VL CDRs that do not have amino acid substitutions, deletions or insertions.

In some embodiments, the SLITRK6 antibodies or antigen binding portions thereof comprise a heavy chain variable (VH) region and a light chain variable (VL) region, the VH and VL regions having amino acid sequences, which have a similarity of at least 80%, at least 85%, at least 90%, at least 95%, at least 98%, or at least 99% with the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34. The levels of similarities result from amino acid substitutions, deletions or insertions to the VH and VL sequences set forth in SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively.

In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the SLITRK6 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% identical with the sequences set forth in SEQ ID NO:1 and SEQ ID NO:2, respectively.

In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable region (VH) and a light chain variable region (VL), the VH and VL regions having the amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the SLITRK6 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% identical with the sequences set forth in SEQ ID NO: 9 and SEQ ID NO: 10, respectively.

In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the SLITRK6 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% identical with the sequences set forth in SEQ ID NO: 17 and SEQ ID NO: 18, respectively.

In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the SLITRK6 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% identical with the sequences set forth in SEQ ID NO: 25 and SEQ ID NO: 26, respectively.

In some embodiments, the SLITRK6 antibodies or antigen binding portions thereof comprise a heavy chain variable (VH) region and a light chain variable (VL) region, the VH and VL regions having the amino acid sequences set forth in and SEQ ID NO: 33 and SEQ ID NO: 34; respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof comprise a heavy chain variable (VH) region and a light chain variable (VL) region, the VH and VL regions having the amino acid sequences set forth in SEQ ID NO: 33 and SEQ ID NO: 34; respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, the SLITRK6 antibodies or antigen binding portions thereof comprise a heavy chain variable (VH) region and a light chain variable (VL) region, the VH and VL regions having the amino acid sequences that are at least 80%, at least 85%, at least 90%, at least 95%, at least 98%, or at least 99% identical with the sequences set forth in SEQ ID NO: 33 and SEQ ID NO: 34, respectively.

In some embodiments, provided herein is a binding agent comprising a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the binding agent specifically binds to human SLITRK6. In some embodiments, the binding agent comprises a heavy chain variable region (VH) and a light chain variable region (VL), the VH and VL regions having the amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the binding agent specifically binds to human SLITRK6 with a higher or a similar binding affinity (lower Kd) than that of antibody sirtratumab. In some embodiments, provided herein is a binding agent comprising a heavy chain variable region (VH) and a light chain variable (VL) region, the VH and VL regions having the amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18; SEQ ID NO: 25 and SEQ ID NO: 26, respectively; and SEQ ID NO: 33 and SEQ ID NO: 34, respectively; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18; respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes a SLITRK6 antibody or antigen binding portion(s) thereof and can optionally include other peptides or polypeptides covalently attached to the SLITRK6 antibody or antigen binding portion thereof. In any of these embodiments, the binding agent specifically binds to human SLITRK6.

In some embodiments, provided herein is a binding agent comprising a heavy chain variable (VH) region and a light chain variable (VL) region, the VH and VL regions having the amino acid sequences set forth in SEQ ID NO: 1 and SEQ ID NO: 2, respectively; wherein the binding agent specifically binds to human SLITRK6. In some embodiments, the binding agent comprises a heavy chain variable (VH) region and a light chain variable (VL) region, the VH and VL regions having amino acid sequences set forth SEQ ID NO: 1 and SEQ ID NO: 2, respectively; wherein the binding agent specifically binds to human SLITRK6 with a higher or a similar binding affinity (lower Kd) than that of antibody sirtratumab. In some embodiments, provided herein is a binding agent comprising a heavy chain variable (VH) region and a light chain variable (VL) region, the VH and VL regions having the amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 9 and SEQ ID NO: 10, respectively; wherein the binding agent specifically binds to human SLITRK6. In some embodiments, the binding agent comprises a heavy chain variable (VH) region and a light chain variable (VL) region, the VH and VL regions having the amino acid sequences set forth in SEQ ID NO: 9 and SEQ ID NO: 10, respectively; wherein the binding agent specifically binds to SLITRK6 with a higher binding affinity (lower Kd) than that of antibody sirtratumab. In some embodiments, provided herein is a binding agent comprising a heavy chain variable (VH) region and a light chain variable (VL) region, the VH and VL regions having the amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the binding agent specifically binds to human SLITRK6. In some embodiments, the binding agent comprises a heavy chain variable (VH) region and a light chain variable (VL) region, the VH and VL regions having the amino acid sequences set forth in SEQ ID NO: 17 and SEQ ID NO: 18, respectively; wherein the binding agent specifically binds to SLITRK6 with a higher binding affinity (lower Kd) than that of antibody sirtratumab. In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 25 and SEQ ID NO: 26, respectively; wherein the binding agent specifically binds to human SLITRK6. In some embodiments, the binding agent comprises a heavy chain variable (VH) region and a light chain variable (VL) region, the VH and VL regions having the amino acid sequences set forth in SEQ ID NO: 25 and SEQ ID NO: 26, respectively; wherein the binding agent specifically binds to SLITRK6 with a higher binding affinity (lower Kd) than that of antibody sirtratumab. In some embodiments, provided herein is a binding agent comprising a heavy chain variable (VH) region and a light chain variable (VL) region, the VH and VL regions having the amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having the amino acid sequences set forth in SEQ ID NO: 33 and SEQ ID NO: 34, respectively; wherein the binding agent specifically binds to human SLITRK6. In some embodiments, the binding agent comprises a heavy chain variable (VH) region and a light chain variable (VL) region, the VH and VL regions having the amino acid sequences set forth in SEQ ID NO: 33 and SEQ ID NO: 34, respectively; wherein the binding agent specifically binds to SLITRK6 with a higher binding affinity (lower Kd) than that of antibody sirtratumab. In some embodiments, provided herein is a binding agent comprising a heavy chain variable (VH) region and a light chain variable (VL) region, the VH and VL regions having the amino acid sequences set forth in SEQ ID NO: 33 and SEQ ID NO: 34, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having the amino acid sequences set forth in SEQ ID NO: 33 and SEQ ID NO: 34, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a binding agent comprising a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the binding agent specifically binds to human SLITRK6. In some embodiments, the binding agent comprises a heavy chain variable region (VH) and a light chain variable region (VL), the VH and VL regions having the amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the binding agent specifically binds to human SLITRK6 with a higher or a similar binding affinity (lower Kd) than that of antibody sirtratumab. In some embodiments, provided herein is a binding agent comprising a heavy chain variable region (VH) and a light chain variable (VL) region, the VH and VL regions having the amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified. As described herein, a binding agent includes a SLITRK6 antibody or antigen binding portion(s) thereof and can optionally include other peptides or polypeptides covalently attached to the SLITRK6 antibody or antigen binding portion thereof. In any of these embodiments, the binding agent specifically binds to human SLITRK6.

In some embodiments, provided herein is a binding agent (e.g., an antibody) comprising a heavy chain and a light chain, wherein the heavy chain and light chain comprise the amino acid sequences set forth in SEQ ID NOs: 53 and 55, respectively. In some embodiments, provided herein is a binding agent (e.g., an antibody) comprising a heavy chain and a light chain, wherein the heavy chain and light chain comprise the amino acid sequences set forth in SEQ ID NOs: 54 and 55, respectively. In some embodiments, provided herein is a binding agent (e.g., an antibody) comprising a heavy chain and a light chain, wherein the heavy chain and light chain comprise the amino acid sequences set forth in SEQ ID NOs: 53 and 55, respectively. In some embodiments, provided herein is a binding agent (e.g., an antibody) comprising a heavy chain and a light chain, wherein the heavy chain and light chain comprise amino acid sequences that are at least 80%, at least 85%, at least 90%, at least 95%, at least 98%, or at least 99% identical to the sequences set forth in SEQ ID NO: 53 and SEQ ID NO: 55, respectively. In some embodiments, provided herein is a binding agent (e.g., an antibody) comprising a heavy chain and a light chain, wherein the heavy chain and light chain comprise amino acid sequences that are at least 80%, at least 85%, at least 90%, at least 95%, at least 98%, or at least 99% identical to the sequences set forth in SEQ ID NO: 54 and SEQ ID NO: 55, respectively. In some embodiments, provided herein is a binding agent (e.g., an antibody) comprising a heavy chain and a light chain, wherein the heavy chain and light chain differ in amino acid sequence from SEQ ID NOs: 53 and 55, respectively, by at most 1-20, 1-10, 1-5, 1-3, 1, 2, 3, 4, 5, 6, 7, 8, 9, 10, 11, 12, 13, 14, 15, 16, 17, 18, 19, or 20 amino acid residues. In some embodiments, provided herein is a binding agent (e.g., an antibody) comprising a heavy chain and a light chain, wherein the heavy chain and light chain differ in amino acid sequence from SEQ ID NOs: 54 and 55, respectively, by at most 1-20, 1-10, 1-5, 1-3, 1, 2, 3, 4, 5, 6, 7, 8, 9, 10, 11, 12, 13, 14, 15, 16, 17, 18, 19, or 20 amino acid residues.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from SEQ ID NO: 3, SEQ ID NO: 4, SEQ ID NO: 5, SEQ ID NO: 6, SEQ ID NO: 7, and SEQ ID NO: 8, respectively; SEQ ID NO: 11, SEQ ID NO: 12, SEQ ID NO: 13, SEQ ID NO: 14, SEQ ID NO: 15 and SEQ ID NO: 16, respectively; SEQ ID NO: 19, SEQ ID NO: 20, SEQ ID NO: 21, SEQ ID NO: 22, SEQ ID NO: 23 and SEQ ID NO: 24, respectively; SEQ ID NO: 27, SEQ ID NO: 28, SEQ ID NO: 29, SEQ ID NO: 30, SEQ ID NO: 31 and SEQ ID NO: 32, respectively; and SEQ ID NO: 35, SEQ ID NO: 36, SEQ ID NO: 37, SEQ ID NO: 38, SEQ ID NO: 39 and SEQ ID NO: 40, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 3, SEQ ID NO: 4, SEQ ID NO: 5, SEQ ID NO: 6, SEQ ID NO: 7, and SEQ ID NO: 8, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 11, SEQ ID NO: 12, SEQ ID NO: 13, SEQ ID NO: 14, SEQ ID NO: 15, and SEQ ID NO: 16, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 19, SEQ ID NO: 20, SEQ ID NO: 21, SEQ ID NO: 22, SEQ ID NO: 23, and SEQ ID NO: 24, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO:27, SEQ ID NO: 28, SEQ ID NO: 29, SEQ ID NO: 30, SEQ ID NO: 31, and SEQ ID NO: 32, respectively. In some embodiments, each VH and VL region comprises a humanized framework region. In some embodiments, each VH and VL region comprises a human framework region.

In some embodiments, provided is an antibody or antigen binding portion comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 35, SEQ ID NO: 36, SEQ ID NO: 37, SEQ ID NO: 38, SEQ ID NO: 39 and SEQ ID NO: 40, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in the sets of amino acid sequences selected from SEQ ID NO: 3, SEQ ID NO: 4, SEQ ID NO: 5, SEQ ID NO: 6, SEQ ID NO: 7, and SEQ ID NO: 8, respectively; SEQ ID NO: 11, SEQ ID NO: 12, SEQ ID NO: 13, SEQ ID NO: 14, SEQ ID NO: 15 and SEQ ID NO: 16, respectively; SEQ ID NO: 19, SEQ ID NO: 20, SEQ ID NO: 21, SEQ ID NO: 22, SEQ ID NO: 23 and SEQ ID NO: 24, respectively; SEQ ID NO: 27, SEQ ID NO: 28, SEQ ID NO: 29, SEQ ID NO: 30, SEQ ID NO: 31 and SEQ ID NO: 32, respectively; and SEQ ID NO: 35, SEQ ID NO: 36, SEQ ID NO: 37, SEQ ID NO: 38, SEQ ID NO: 39 and SEQ ID NO: 40, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 3, SEQ ID NO: 4, SEQ ID NO: 5, SEQ ID NO: 6, SEQ ID NO: 7, and SEQ ID NO: 8, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 11, SEQ ID NO: 12, SEQ ID NO: 13, SEQ ID NO: 14, SEQ ID NO: 15, and SEQ ID NO: 16, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 19, SEQ ID NO: 20, SEQ ID NO: 21, SEQ ID NO: 22, SEQ ID NO: 23, and SEQ ID NO: 24, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 27, SEQ ID NO: 28, SEQ ID NO: 29, SEQ ID NO: 30, SEQ ID NO: 31, and SEQ ID NO: 32, respectively. In some embodiments, each VH and VL region comprises a humanized framework region. In some embodiments, each VH and VL region comprises a human framework region.

In some embodiments, provided is a binding agent comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having the amino acids sequences set forth in SEQ ID NO: 35, SEQ ID NO: 36, SEQ ID NO: 37, SEQ ID NO: 38, SEQ ID NO: 39 and SEQ ID NO: 40, respectively. In some embodiments, each VH and VL region comprises a humanized framework region. In some embodiments, each VH and VL region comprises a human framework region.

In some embodiments, the compositions and methods described herein relate to reduction of SLITRK6+ cells in a subject (e.g., reducing the number of SLITRK6+ cells in a cancer or tumor, or SLITRK6+ cells associated with an autoimmune disease or disorder) by a SLITRK6 antibody, antigen binding portion thereof, other binding agent or conjugate thereof in vivo. In some embodiments, the compositions and methods described herein relate to the treatment of SLITRK6+ cancer in a subject by administering a SLITRK6 antibody, antigen binding portion thereof, other binding agent or conjugate thereof. In some embodiments, the compositions and methods described herein relate to the treatment of an autoimmune disorder in a subject by administering a SLITRK6 antibody, antigen binding portion thereof, other binding agent or conjugate thereof. In some embodiments, the compositions and methods described herein relate to the treatment of disease or disorder associated with SLITRK6+ cells in a subject by administering a SLITRK6 antibody, antigen binding portion thereof, other binding agent or conjugate thereof. In any of these embodiments, the methods further include a reduction in the number of SLITRK6+ cells in the subject that are associated with the disease, condition or cancer.

As used herein, the term “antibody” refers to immunoglobulin molecules and immunologically active portions of immunoglobulin molecules, i.e., molecules that contain an antigen binding site(s) that specifically binds to an antigen, e.g., human SLITRK6. The term generally refers to antibodies comprised of two immunoglobulin heavy chain variable regions and two immunoglobulin light chain variable regions including full length antibodies (having heavy and light chain constant regions).

Each heavy chain is composed of a variable region (abbreviated as VH) and a constant region. The heavy chain constant region may include three domains CH1, CH2 and CH3 and optionally a fourth domain, CH4. Each light chain is composed of a variable region (abbreviated as VL) and a constant region. The light chain constant region is a CL domain. The VH and VL regions may be further divided into hypervariable regions referred to as complementarity-determining regions (CDRs) and interspersed with conserved regions referred to as framework regions (FR). Each VH and VL region thus consists of three CDRs and four FRs that are arranged from the N terminus to the C terminus in the following order: FR1, CDR1, FR2, CDR2, FR3, CDR3, and FR4. This structure is well known to those skilled in the art.

As used herein, an “antigen-binding portion” of a SLITRK6 antibody refers to the portions of a SLITRK6 antibody as described herein having the VH and VL sequences of the SLITRK6 antibody or the CDRs of a SLITRK6 antibody and that specifically binds to SLITRK6. Examples of antigen binding portions include a Fab, a Fab′, a F(ab′)2, a Fv, a scFv, a disulfide linked Fv, a single domain antibody (also referred to as a VHH, VNAR, sdAb, or nanobody) or a diabody (see, e.g., Huston et al., Proc. Natl. Acad. Sci. U.S.A., 85, 5879-5883 (1988) and Bird et al., Science 242, 423-426 (1988), which are incorporated herein by reference). As used herein, the terms Fab, F(ab′)2 and Fv refer to the following: (i) a Fab fragment, i.e. a monovalent fragment composed of the VL, VH, CL and CH₁ domains; (ii) an F(ab′)2 fragment, i.e. a bivalent fragment comprising two Fab fragments linked to one another in the hinge region via a disulfide bridge; and (iii) an Fv fragment composed of the VL and VH domains, in each case of a SLITRK6 antibody. Although the two domains of the Fv fragment, namely VL and VH, are encoded by separate coding regions, they may further be linked to one another using a synthetic linker, e.g., a poly-G4S amino acid sequence (‘(G4S)n’ disclosed as SEQ ID NO: 56, wherein n=1 to 5), making it possible to prepare them as a single protein chain in which the VL and VH regions combine in order to form monovalent molecules (known as single chain Fv or scFv). The term “antigen-binding portion” of an antibody is also intended to include such single chain antibodies. Other forms of single chain antibodies such as “diabodies” are likewise included here. Diabodies are bivalent, bispecific antibodies in which VH and VL domains are expressed on a single polypeptide chain, but using a linker connecting the VH and VL domains that is too short for the two domains to be able to combine on the same chain, thereby forcing the VH and VL domains to pair with complementary domains of a different chain (VL and VH, respectively), and to form two antigen-binding sites (see, for example, Holliger, R, et al. (1993) Proc. Natl. Acad. Sci. USA 90:64446448; Poljak, R. J, et al. (1994) Structure 2:1121-1123).

A single-domain antibody is an antibody portion consisting of a single monomeric variable antibody domain. Single domains antibodies can be derived from the variable domain of the antibody heavy chain from camelids (e.g., nanobodies or VHH portions). Furthermore, the term single-domain antibody includes an autonomous human heavy chain variable domain (aVH) or VNAR portions derived from sharks (see, e.g., Hasler et al., Mol. Immunol. 75:28-37, 2016).

Techniques for producing single domain antibodies (e.g., DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with novel epitopes that are inaccessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize a target antigen (see, e.g., Maass et al., 2007). PCR primers that amplify alpaca VHH coding sequences have been identified and may be used to construct alpaca VHH phage display libraries, which can be used for antibody fragment isolation by standard biopanning techniques well known in the art (see, e.g., Maass et al., 2007).

In some embodiments, the SLITRK6 antibodies or antigen binding portions thereof are part of a bispecific or multispecific binding agent. Bispecific and multi-specific antibodies include the following: an scFv1-ScFv2, an ScFv12-Fc-scFv22, an IgG-scFv, a DVD-Ig, a triomab/quadroma, a two-in-one IgG, a scFv2-Fc, a TandAb, and an scFv-HSA-scFv. In some embodiments, an IgG-scFv is an IgG(H)-scFv, scFv-(H)IgG, IgG(L)-scFv, svFc-(L)IgG, 2scFV-IgG or IgG-2scFv. See, e.g., Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

### Modification of VH and VL Regions

As to the VH and VL amino acid sequences, one of skill will recognize that individual substitutions, deletions or additions (insertions) to a nucleic acid encoding the VH or VL, or amino acids in a polypeptide that alter a single amino acid or a small percentage of amino acids in the encoded sequence is a “conservatively modified variant”, where the alteration results in the substitution of an amino acid with a chemically similar amino acid (a conservative amino acid substitution) and the altered polypeptide retains the ability to specifically bind to SLITRK6.

In some embodiments, a conservatively modified variant of a SLITRK6 antibody or antigen binding portion thereof can have an alteration(s) in the framework regions (i.e., other than in the CDRs), e.g. a conservatively modified variant of a SLITRK6 antibody has the amino acid sequences of the VH and VL CDRs (set forth in sets of amino acid sequences (i) SEQ ID NO: 3, SEQ ID NO: 4, SEQ ID NO: 5, SEQ ID NO: 6, SEQ ID NO: 7, and SEQ ID NO: 8, respectively; (ii) SEQ ID NO: 11, SEQ ID NO: 12, SEQ ID NO: 13, SEQ ID NO: 14, SEQ ID NO: 15 and SEQ ID NO: 16, respectively; (iii) SEQ ID NO: 19, SEQ ID NO: 20, SEQ ID NO: 21, SEQ ID NO: 22, SEQ ID NO: 23 and SEQ ID NO: 24, respectively; (iv) SEQ ID NO: 27, SEQ ID NO: 28, SEQ ID NO: 29, SEQ ID NO: 30, SEQ ID NO: 31 and SEQ ID NO: 32, respectively; and (v) SEQ ID NO: 35, SEQ ID NO: 36, SEQ ID NO: 37, SEQ ID NO: 38, SEQ ID NO: 39 and SEQ ID NO: 40, respectively) and has at least one conservative amino acid substitution in a framework region (FR). In some embodiments, the VH and VL amino acid sequences collectively have no more than 8 or 6 or 4 or 2 or 1 conservative amino acid substitutions in the FR, as compared to the amino acid sequences of the unmodified VH and VL regions. In some embodiments, the VH and VL amino acid sequences have 8 to 1, 6 to 1, 4 to 1 or 2 to 1 conservative amino acid substitutions in the FR, as compared to the amino acid sequences of the unmodified VH and VL regions. In further aspects of any of these embodiments, a conservatively modified variant of the SLITRK6 antibody, antigen binding portion thereof or other binding agent exhibits specific binding to SLITRK6.

For conservative amino acid substitutions, a given amino acid can be replaced by a residue having similar physiochemical characteristics, e.g., substituting one aliphatic residue for another (such as lie, Val, Leu, or Ala for one another), or substitution of one polar residue for another (such as between Lys and Arg; Glu and Asp; or Gln and Asn). Other such conservative amino acid substitutions, e.g., substitutions of entire regions having similar hydrophobicity characteristics, are well known. Polypeptides comprising conservative amino acid substitutions can be tested in any one of the assays described herein to confirm that a desired activity, e.g. antigen-binding activity and specificity of a native or reference polypeptide is retained, i.e., to SLITRK6.

In some embodiments, a SLITRK6 antibody or antigen binding portion thereof or other binding agent can be further optimized to, for example, decrease potential immunogenicity or optimize other functional property, while maintaining functional activity, for therapy in humans. In some embodiments, the SLITRK6 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 conservative amino acid substitutions in the framework regions, wherein the CDRs of the heavy or light chain variable regions are not modified. In some embodiments, the SLITRK6 antibodies or antigen binding portions thereof or other binding agents comprise a heavy chain variable (VH) region and a light chain variable (VL) region, the VH and VL regions having amino acid sequences set forth in the pairs of amino acid sequences selected from SEQ ID NO: 1 and SEQ ID NO: 2, respectively; SEQ ID NO: 9 and SEQ ID NO: 10, respectively; SEQ ID NO: 17 and SEQ ID NO: 18, respectively; SEQ ID NO: 25 and SEQ ID NO: 26, respectively; and SEQ ID NO: 33 and SEQ ID NO: 34, respectively; wherein the heavy and light chain variable framework regions are optionally modified with from 1 to 8, 1 to 6, 1 to 4 or 1 to 2 amino acid substitutions, deletions or insertions in the framework regions, wherein the CDRs of the heavy or light chain variable regions are not modified.

In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 1 and SEQ ID NO: 2, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 9 and SEQ ID NO: 10, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 17 and SEQ ID NO: 18, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a SLITRK6 antibody or antigen binding portion thereof or other binding agent comprising a heavy chain variable (VH) region and a light chain variable (VL) region, the VH and VL regions having amino acid sequences set forth in SEQ ID NO: 25 and SEQ ID NO: 26, respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In some embodiments, provided herein is a SLITRK6 antibody or antigen binding portion thereof or other binding agent comprising a heavy chain variable region (VH) and a light chain variable region (VL), the VH and VL regions having amino acid sequences set forth in SEQ ID NO: 33 and SEQ ID NO: 34; respectively; wherein the heavy and light chain variable framework regions are optionally modified with from 1 to 8, 1 to 6, 1 to 4 or 1 to 2 conservative amino acid substitutions in the framework regions and wherein the CDRs of the heavy or light chain variable regions are not modified. In some embodiments, provided herein is a binding agent comprising a heavy chain variable region (VH) and a light chain variable region (VL), the VH and VL regions having amino acid sequences set forth in SEQ ID NO: 33 and SEQ ID NO: 34; respectively; wherein the heavy and light chain variable framework regions are optionally modified with from 1 to 8, 1 to 6, 1 to 4 or 1 to 2 amino acid substitutions, deletions or insertions in the framework regions and wherein the CDRs of the heavy or light chain variable regions are not modified.

In any of these embodiments, the functional activity of the SLITRK6 binding antibody or antigen binding portion thereof or other binding agent includes specifically binding to SLITRK6. Additional functional activities include depletion of SLITRK6+ cells (e.g., cancer cells or autoimmune cells). In the case where dose dependency does exist, it needs not be identical to that of the reference antibody or antigen-binding portion thereof, but rather substantially similar to or better than the dose-dependence in a given activity as compared to the reference antibody or antigen-binding portion thereof as described herein (i.e., the candidate polypeptide will exhibit greater activity relative to the reference antibody).

For conservative substitutions, amino acids can be grouped according to similarities in the properties of their side chains (in A. L. Lehninger, in Biochemistry, second ed., pp. 73-75, Worth Publishers, New York (1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H).

Alternatively, for conservative substitutions naturally occurring residues can be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe. Non-conservative substitutions will entail exchanging a member of one of these classes or another class.

Particular conservative substitutions include, for example; Ala to Gly or to Ser; Arg to Lys; Asn to Gln or to His; Asp to Glu; Cys to Ser; Gln to Asn; Glu to Asp; Gly to Ala or to Pro; His to Asn or to Gln; Ile to Leu or to Val; Leu to Ile or to Val; Lys to Arg, to Gln or to Glu; Met to Leu, to Tyr or to lie; Phe to Met, to Leu or to Tyr; Ser to Thr; Thr to Ser; Trp to Tyr; Tyr to Trp; and/or Phe to Val, to Ile or to Leu.

In some embodiments, a conservatively modified variant of a SLITRK6 antibody or antigen binding portion thereof preferably is at least 90%, at least 91%, at least 92%, at least 93%, at least 94%, at least 95%, at least 96%, at least 97%, at least 98%, at least 99%, or more, identical to the reference VH or VL sequence, wherein the VH and VL CDRs are not modified. The degree of homology (percent identity) between the reference and modified sequence can be determined, for example, by comparing the two sequences using freely available computer programs commonly employed for this purpose on the world wide web (e.g., BLASTp or BLASTn with default settings).

In some embodiments, the VH and VL amino acid sequences collectively have no more than 8 or 6 or 4 or 2 or 1 conservative amino acid substitutions in the framework regions, as compared to the amino acid sequences of the unmodified VH and VL regions. In some embodiments, the VH and VL amino acid sequences collectively have 8 to 1, or 6 to 1, or 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions in the framework regions, as compared to the amino acid sequences of the unmodified VH and VL regions. In some embodiments, the VH and VL amino acid sequences have 8 to 1, 6 to 1, 4 to 1, or 2 to 1 conservative amino acid substitutions in the framework regions, as compared to the amino acid sequences of the unmodified VH and VL regions. In some embodiments, the VH and VL amino acid sequences collectively have no more than 8 or 6 or 4 or 2 or 1 amino acid substitutions, deletions or insertions, as compared to the amino acid sequences of the unmodified VH and VL regions.

Modification of a native (or reference) amino acid sequence can be accomplished by any of a number of techniques known to one of skill in the art. Mutations can be introduced, for example, at particular loci by synthesizing oligonucleotides containing the desired mutant sequence, flanked by restriction sites enabling ligation to fragments of the native sequence. Following ligation, the resulting reconstructed sequence encodes a variant having the desired amino acid insertion, substitution, or deletion. Alternatively, oligonucleotide-directed site-specific mutagenesis procedures can be employed to provide an altered nucleotide sequence having particular codons altered according to the substitution, deletion, or insertion desired. Techniques for making such alterations are very well established and include, for example, those disclosed by Walder et al. (Gene 42:133, 1986); Bauer et al. (Gene 37:73, 1985); Craik (BioTechniques, January 1985, 12-19); Smith et al. (Genetic Engineering: Principles and Methods, Plenum Press, 1981); and U.S. Pat. Nos. 4,518,584 and 4,737,462, which are herein incorporated by reference in their entireties.

### Constant Regions

In some embodiments, a SLITRK6 antibody or antigen-binding portion thereof or other binding agent has fully human constant regions. In some embodiments, a SLITRK6 antibody or antigen-binding portion thereof or other binding agent has humanized constant regions. In some embodiments, a SLITRK6 antibody or antigen-binding portion thereof or other binding agent has non-human constant regions. An immunoglobulin constant region refers to a heavy or light chain constant region. Human heavy chain and light chain constant region amino acid sequences are known in the art. A constant region can be of any suitable type, which can be selected from the classes of immunoglobulins, IgA, IgD, IgE, IgG, and IgM. Several immunoglobulin classes can be further divided into isotypes, e.g., IgG1, IgG2, IgG3, IgG4, or IgA1, and IgA2. The heavy-chain constant regions (Fc) that correspond to the different classes of immunoglobulins can be α, δ, ε, γ, and μ, respectively. The light chains can be one of either kappa (or κ) and lambda (or λ).

In some embodiments, a constant region can have an IgG1 isotype. In some embodiments, a constant region can have an IgG2 isotype. In some embodiments, a constant region can have an IgG3 isotype. In some embodiments, a constant region can have an IgG4 isotype. In some embodiments, an Fc domain can have a hybrid isotype comprising constant regions from two or more isotypes. In some embodiments, an immunoglobulin constant region can be an IgG1 or IgG4 constant region. In some embodiments, a SLITRK6 antibody heavy chain is of the IgG1 isotype and has the amino acid sequence set forth in SEQ ID NO: 49. In some embodiments, a SLITRK6 antibody heavy chain is of the IgG1 isotype and has the amino acid sequence set forth in SEQ ID NO: 51. In some embodiments, a SLITRK6 antibody heavy chain is of the IgG1 isotype and has the amino acid sequence set forth in SEQ ID NO: 52. In some embodiments, a SLITRK6 antibody light chain is of the kappa isotype and has the amino acid sequence set forth in SEQ ID NO: 50.

Furthermore, a SLITRK6 antibody or an antigen-binding portion thereof or other binding agent may be part of a larger binding agent formed by covalent or noncovalent association of the antibody or antigen binding portion with one or more other proteins or peptides. Relevant to such binding agents are the use, for example, of the streptavidin core region in order to prepare a tetrameric scFv molecule (Kipriyanov, S. M., et al. (1995), Human Antibodies and Hybridomas 6:93-101) and the use of a cysteine residue, a marker peptide and a C-terminal polyhistidinyl peptide, e.g. hexahistidinyl tag in order to produce bivalent and biotinylated scFv molecules (Kipriyanov, S. M., et al. (1994) Mol. Immunol. 31:10471058).

Fc regions may have at their C-terminus a lysine. The origin of this lysine is a naturally occurring sequence found in humans from which these Fc regions are derived. During cell culture production of recombinant antibodies, this terminal lysine can be cleaved off by proteolysis by endogenous carboxypeptidase(s), resulting in a constant region having the same sequence but lacking the C-terminal lysine. For manufacturing purposes of antibodies, the DNA encoding this terminal lysine can be omitted from the sequence such that antibodies are produced without the lysine. Antibodies produced from nucleic acid sequences that either do, or do not encode a terminal lysine are substantially identical in sequence and in function since the degree of processing of the terminal lysine is typically high when e.g. using antibodies produced in CHO-based production systems (Dick, L. W. et al. Biotechnol. Bioeng. 2008; 100: 1132-1143). Hence, it is understood that proteins in accordance with the invention, such as antibodies, can be generated with or without encoding or having a terminal lysine. It is also understood in accordance with the invention that, sequences with a terminal lysine, such as a constant region sequence having a terminal lysine, can be understood as the corresponding sequences without a terminal lysine, and that sequences without a terminal lysine can also be understood as the corresponding sequences with a terminal lysine.

### Fc Domain Modifications to Alter Effector Function

In some embodiments, an Fc region or Fc domain of a SLITRK6 antibody or antigen binding portion thereof or other binding agent has substantially no binding to at least one Fc receptor selected from FcyRI (CD64), FcyRIIA (CD32a), FcyRIIB (CD32b), FcyRIIIA (CD16a), and FcyRIIIB (CD16b). In some embodiments, an Fc region or domain exhibits substantially no binding to any of the Fc receptors selected from FcyRI (CD64), FcyRIIA (CD32a), FcyRIIB (CD32b), FcyRIIIA (CD16a), and FcyRIIIB (CD16b). As used herein, “substantially no binding” refers to weak to no binding to a selected Fcgamma receptor or receptors. In some embodiments, “substantially no binding” refers to a reduction in binding affinity (i.e., increase in Kd) to a Fc gamma receptor of at least 1000-fold. In some embodiments, an Fc domain or region is an Fc null. As used herein, an “Fc null” refers to an Fc region or Fc domain that exhibits weak to no binding to any of the Fcgamma receptors. In some embodiments, an Fc null domain or region exhibits a reduction in binding affinity (i.e., increase in Kd) to Fc gamma receptors of at least 1000-fold.

In some embodiments, an Fc domain has reduced or substantially no effector function activity. As used herein, “effector function activity” refers to antibody dependent cellular cytotoxicity (ADCC), antibody dependent cellular phagocytosis (ADCP) and/or complement dependent cytotoxicity (CDC). In some embodiments, an Fc domain exhibits reduced ADCC, ADCP or CDC activity, as compared to a wildtype Fc domain. In some embodiments, an Fc domain exhibits a reduction in ADCC, ADCP and CDC, as compared to a wildtype Fc domain.

In some embodiments, an Fc domain exhibits substantially no effector function (i.e., the ability to stimulate or effect ADCC, ADCP or CDC). As used herein, “substantially no effector function” refers to a reduction in effector function activity of at least 1000-fold, as compared to a wildtype or reference Fc domain.

In some embodiments, an Fc domain has reduced or no ADCC activity. As used herein reduced or no ADCC activity refers to a decrease in ADCC activity of an Fc domain by a factor of at least 10, at least 20, at least 30, at least 50, at least 100 or at least 500.

In some embodiments, an Fc domain has reduced or no CDC activity. As used herein reduced or no CDC activity refers to a decrease in CDC activity of an Fc domain by of a factor of at least 10, at least 20, at least 30, at least 50, at least 100 or at least 500.

In vitro and/or in vivo cytotoxicity assays can be conducted to confirm the reduction/depletion of ADCC and/or CDC activity. For example, Fc receptor (FcR) binding assays can be conducted to ensure that the antibody lacks Fcgamma receptor binding (hence likely lacking ADCC activity). The primary cells for mediating ADCC, NK cells, express FcgammaRIII only, whereas monocytes express FcgammaRI, FcgammaRII and FcgammaRIII. FcR expression on hematopoietic cells is summarized in Table 3 on page 464 of Ravetch and Kinet, Annu. Rev. Immunol. 9:457-492 (1991). Non-limiting examples of in vitro assays to assess ADCC activity of a molecule of interest are described in U.S. Pat. No. 5,500,362 (see, e.g. Hellstrom, I. et al. Proc. Nat'l Acad. Sci. USA 83:7059-7063 (1986)) and Hellstrom, I et al., Proc. Nat'l Acad. Sci. USA 82:1499-1502 (1985); U.S. Pat. No. 5,821,337 (see Bruggemann, M. et al., J. Exp. Med. 166:1351-1361 (1987)). Alternatively, non-radioactive assay methods may be employed (see, for example, ACTITM non-radioactive cytotoxicity assay for flow cytometry (CellTechnology, Inc. Mountain View, Calif.; and CytoTox 96™ non-radioactive cytotoxicity assay (Promega, Madison, Wis.). Useful effector cells for such assays include peripheral blood mononuclear cells (PBMC) and Natural Killer (NK) cells. Alternatively, or additionally, ADCC activity of the molecule of interest may be assessed in vivo, e.g., in an animal model such as that disclosed in Clynes et al., Proc. Nat'l Acad. Sci. USA 95:652-656 (1998).

C1q binding assays may also be carried out to confirm that an antibody or Fc domain or region is unable to bind C1q and hence lacks CDC activity or has reduced CDC activity. See, e.g., C1q and C3c binding ELISA in WO 2006/029879 and WO 2005/100402. To assess complement activation, a CDC assay may be performed (see, for example, Gazzano-Santoro et al., J. Immunol. Methods 202:163 (1996); Cragg, M. S. et al., Blood 101:1045-1052 (2003); and Cragg, M. S. and M. J. Glennie, Blood 103:2738-2743 (2004)).

In some embodiments, an Fc domain has reduced or no ADCP activity. As used herein reduced or no ADCP activity refers to a decrease in ADCP activity of an Fc domain by a factor of at least 10, at least 20, at least 30, at least 50, at least 100 or at least 500.

ADCP binding assays may also be carried out to confirm that an antibody or Fc domain or region lacks ADCP activity or has reduced ADCP activity. See, e.g., US20190079077 and US20190048078 and the references disclosed therein.

A SLITRK6 antibody or antigen binding portion thereof or other binding agent with reduced effector function activity includes those with substitution of one or more of Fc region residues, such as, for example, 238, 265, 269, 270, 297, 327 and 329, according to the EU number of Kabat (see, e.g., U.S. Pat. No. 6,737,056). Such Fc mutants include Fc mutants with substitutions at two or more of amino acid positions 265, 269, 270, 297 and 327, including the so-called “DANA” Fc mutant with substitution of residues 265 and 297 to alanine, according to the EU numbering of Kabat (see U.S. Pat. No. 7,332,581). Certain antibody variants with diminished binding to FcRs are also known. (See, e.g., U.S. Pat. No. 6,737,056; WO 2004/056312, and Shields et al., J. Biol. Chem. 9(2): 6591-6604 (2001).) A SLITRK6 antibody or antigen binding portion thereof or other binding agent with diminished binding to FcRs can be prepared containing such amino acid modifications.

In some embodiments, a SLITRK6 antibody or antigen binding portion thereof or other binding agent comprises an Fc domain or region with one or more amino acid substitutions which diminish FcgammaR binding, e.g., substitutions at positions 234 and 235 of the Fc region (EU numbering of residues). In some embodiments, the substitutions are L234A and L235A (LALA), according to the EU numbering of Kabat. In some embodiments, the Fc domain comprises D265A and/or P329G in an Fc region derived from a human IgG1 Fc region, according to the EU numbering of Kabat. In some embodiments, the substitutions are L234A, L235A and P329G (LALA-PG), according to the EU numbering of Kabat, in an Fc region derived from a human IgG1 Fc region. (See, e.g., WO 2012/130831). In some embodiments, the substitutions are L234A, L235A and D265A (LALA-DA) in an Fc region derived from a human IgG1 Fc region, according to the EU numbering of Kabat. In some embodiments, the SLITRK6 antibody or antigen binding portion thereof or other binding agent comprises a human IgG1 Fc region comprising a L234A mutation and an L235A mutation (LALA), wherein the amino acid positions are numbered according to Eu numbering. In some embodiments, the SLITRK6 antibody or antigen binding portion thereof or other binding agent comprises a heavy chain constant region comprising the sequence set forth in SEQ ID NO: 51. In some embodiments, the SLITRK6 antibody or antigen binding portion thereof or other binding agent comprises a heavy chain constant region comprising the sequence set forth in SEQ ID NO: 51 and a light chain constant region comprising the sequence set forth in SEQ ID NO: 50. In some embodiments, the SLITRK6 antibody or antigen binding portion thereof or other binding agent comprises a heavy chain constant region comprising the sequence set forth in SEQ ID NO: 52. In some embodiments, the SLITRK6 antibody or antigen binding portion thereof or other binding agent comprises a heavy chain constant region comprising the sequence set forth in SEQ ID NO: 52 and a light chain constant region comprising the sequence set forth in SEQ ID NO: 50. In some embodiments, the SLITRK6 antibody or antigen binding portion thereof or other binding agent comprises a heavy chain comprising the sequence set forth in SEQ ID NO: 53. In some embodiments, the SLITRK6 antibody or antigen binding portion thereof or other binding agent comprises a heavy chain comprising the sequence set forth in SEQ ID NO: 53 and a light chain comprising the sequence set forth in SEQ ID NO: 55. In some embodiments, the SLITRK6 antibody or antigen binding portion thereof or other binding agent comprises a heavy chain comprising the sequence set forth in SEQ ID NO: 54. In some embodiments, the SLITRK6 antibody or antigen binding portion thereof or other binding agent comprises a heavy chain comprising the sequence set forth in SEQ ID NO: 54 and a light chain comprising the sequence set forth in SEQ ID NO: 55.

In some embodiments, alterations are made in the Fc region that result in altered (i.e., either diminished) C1q binding and/or Complement Dependent Cytotoxicity (CDC), e.g., as described in U.S. Pat. No. 6,194,551, WO 99/51642, and Idusogie et al. J. Immunol. 164: 4178-4184 (2000).

### Methods of Making Antibodies, Antigen Binding Portions and Other Binding Agents

In various embodiments, SLITRK6 antibodies, antigen binding portions thereof and other binding agents can be produced in human, murine or other animal-derived cells lines. Recombinant DNA expression can be used to produce SLITRK6 antibodies, antigen binding portions thereof and other binding agents. This allows the production of SLITRK6 antibodies as well as a spectrum of SLITRK6 antigen binding portions and other binding agents (including fusion proteins) in a host species of choice. The production of SLITRK6 antibodies, antigen binding portions thereof and other binding agents in bacteria, yeast, transgenic animals and chicken eggs are also alternatives for cell-based production systems. The main advantages of transgenic animals are potential high yields from renewable sources.

In some embodiments, a SLITRK6 VH polypeptide having the amino acid sequence set forth in SEQ ID NOs:1, 9, 17, 25, and 33 is encoded by a nucleic acid. In some embodiments, a SLITRK6 VL polypeptide having the amino acid sequence set forth in SEQ ID NOs: 2, 10, 18, 26, and 34 is encoded by a nucleic acid. In some embodiments, a nucleic acid encodes a SLITRK6 VH polypeptide having the amino acid sequence set forth in SEQ ID NOs: 1, 9, 17, 25, and 33. In some embodiments, a nucleic acid encodes a SLITRK6 VL polypeptide having the amino acid sequence set forth in SEQ ID NOs: 2, 10, 18, 26, and 34. In some embodiments, the nucleic acid encodes a SLITRK6 VH polypeptide having the amino acid sequence set forth in SEQ ID NO: 1. In some embodiments, the nucleic acid encodes a SLITRK6 VH polypeptide having the amino acid sequence set forth in SEQ ID NO: 9. In some embodiments, the nucleic acid encodes a SLITRK6 VH polypeptide having the amino acid sequence set forth in SEQ ID NO: 17. In some embodiments, the nucleic acid encodes a SLITRK6 VH polypeptide having the amino acid sequence set forth in SEQ ID NO: 25. In some embodiments, the nucleic acid encodes a SLITRK6 VH polypeptide having the amino acid sequence set forth in SEQ ID NO: 33. In some embodiments, the nucleic acid encodes a SLITRK6 VH polypeptide having the amino acid sequence set forth in SEQ ID NO: 2. In some embodiments, the nucleic acid encodes a SLITRK6 VH polypeptide having the amino acid sequence set forth in SEQ ID NO: 10. In some embodiments, the nucleic acid encodes a SLITRK6 VH polypeptide having the amino acid sequence set forth in SEQ ID NO: 18. In some embodiments, the nucleic acid encodes a SLITRK6 VH polypeptide having the amino acid sequence set forth in SEQ ID NO: 26. In some embodiments, the nucleic acid encodes a SLITRK6 VH polypeptide having the amino acid sequence set forth in SEQ ID NO: 34.

In some embodiments, the nucleic acid encodes VH and VL polypeptides having the amino acid sequences set forth in SEQ ID NOs: 1 and 2. In some embodiments, the nucleic acid encodes VH and VL polypeptides having the amino acid sequences set forth in SEQ ID NOs: 9 and 10. In some embodiments, the nucleic acid encodes VH and VL polypeptides having the amino acid sequences set forth in SEQ ID NOs: 17 and 18. In some embodiments, the nucleic acid encodes VH and VL polypeptides having the amino acid sequences set forth in SEQ ID NOs: 25 and 26. In some embodiments, the nucleic acid encodes VH and VL polypeptides having the amino acid sequences set forth in SEQ ID NOs: 33 and 34.

In some embodiments, the nucleic acid encodes heavy chain polypeptide having the amino acid sequence set forth in SEQ ID NO: 53. In some embodiments, the nucleic acid encodes heavy chain polypeptide having the amino acid sequence set forth in SEQ ID NO: 54. In some embodiments, the nucleic acid encodes light chain polypeptide having the amino acid sequence set forth in SEQ ID NO: 55. In some embodiments, the nucleic acid encodes heavy and light chain polypeptides having the amino acid sequences set forth in SEQ ID NOs: 53 and 55. In some embodiments, the nucleic acid encodes heavy and light chain polypeptides having the amino acid sequences set forth in SEQ ID NOs: 54 and 55.

As used herein, the term “nucleic acid” or “nucleic acid sequence” or “polynucleotide sequence” or “nucleotide” refers to a polymeric molecule incorporating units of ribonucleic acid, deoxyribonucleic acid or an analog thereof. The nucleic acid can be either single-stranded or double-stranded. A single-stranded nucleic acid can be one strand nucleic acid of a denatured double-stranded DNA. In some embodiments, the nucleic acid can be a cDNA, e.g., a nucleic acid lacking introns.

Nucleic acid molecules encoding the amino acid sequence of a SLITRK6 antibody, antigen binding portion thereof as well as other binding agents can be prepared by a variety of methods known in the art. These methods include, but are not limited to, preparation of synthetic nucleotide sequences encoding of a SLITRK6 antibody, antigen binding portion or other binding agent(s). In addition, oligonucleotide-mediated (or site-directed) mutagenesis, PCR-mediated mutagenesis, and cassette mutagenesis can be used to prepare nucleotide sequences encoding a SLITRK6 antibody or antigen binding portion as well as other binding agents. A nucleic acid sequence encoding at least a SLITRK6 antibody, antigen binding portion thereof, binding agent, or a polypeptide thereof, as described herein, can be recombined with vector DNA in accordance with conventional techniques, such as, for example, blunt-ended or staggered-ended termini for ligation, restriction enzyme digestion to provide appropriate termini, filling in of cohesive ends as appropriate, alkaline phosphatase treatment to avoid undesirable joining, and ligation with appropriate ligases or other techniques known in the art. Techniques for such manipulations are disclosed, e.g., by Maniatis et al., Molecular Cloning, Lab. Manual (Cold Spring Harbor Lab. Press, NY, 1982 and 1989), and Ausubel et al., Current Protocols in Molecular Biology (John Wiley & Sons), 1987-1993, and can be used to construct nucleic acid sequences and vectors that encode a SLITRK6 antibody or antigen binding portion thereof or a VH or VL polypeptide thereof or other binding agent.

A nucleic acid molecule, such as DNA, is said to be “capable of expressing” a polypeptide if it contains nucleotide sequences that contain transcriptional and translational regulatory information and such sequences are “operably linked” to nucleotide sequences that encode the polypeptide. An operable linkage is a linkage in which the regulatory DNA sequences and the DNA sequence sought to be expressed (e.g., a SLITRK6 antibody or antigen binding portion thereof or other binding agent) are connected in such a way as to permit gene expression of a polypeptide(s) or antigen binding portions in recoverable amounts. The precise nature of the regulatory regions needed for gene expression may vary from organism to organism, as is well known in the analogous art. See, e.g., Sambrook et al., 1989; Ausubel et al., 1987-1993.

Accordingly, the expression of a SLITRK6 antibody or antigen-binding portion thereof as described herein can occur in either prokaryotic or eukaryotic cells. Suitable hosts include bacterial or eukaryotic hosts, including yeast, insects, fungi, bird and mammalian cells either in vivo or in situ, or host cells of mammalian, insect, bird or yeast origin. The mammalian cell or tissue can be of human, primate, hamster, rabbit, rodent, cow, pig, sheep, horse, goat, dog or cat origin, but any other mammalian cell may be used. Further, by use of, for example, the yeast ubiquitin hydrolase system, in vivo synthesis of ubiquitin-transmembrane polypeptide fusion proteins can be accomplished. The fusion proteins so produced can be processed in vivo or purified and processed in vitro, allowing synthesis of a SLITRK6 antibody or antigen binding portion thereof or other binding agent as described herein with a specified amino terminus sequence. Moreover, problems associated with retention of initiation codon-derived methionine residues in direct yeast (or bacterial) expression maybe avoided. (See, e.g., Sabin et al., 7 Bio/Technol. 705 (1989); Miller et al., 7 Bio/Technol. 698 (1989).) Any of a series of yeast gene expression systems incorporating promoter and termination elements from the actively expressed genes coding for glycolytic enzymes produced in large quantities when yeast are grown in medium rich in glucose can be utilized to obtain recombinant SLITRK6 antibodies or antigen-binding portions thereof or other binding agents. Known glycolytic genes can also provide very efficient transcriptional control signals. For example, the promoter and terminator signals of the phosphoglycerate kinase gene can be utilized.

Production of SLITRK6 antibodies or antigen-binding portions thereof or other binding agents in insects can be achieved, for example, by infecting an insect host with a baculovirus engineered to express a polypeptide by methods known to those of ordinary skill in the art. See Ausubel et al., 1987-1993.

In some embodiments, the introduced nucleic acid sequence(s) (encoding a SLITRK6 antibody or antigen binding portion thereof or other binding agent or a polypeptide thereof) is incorporated into a plasmid or viral vector capable of autonomous replication in a recipient host cell. Any of a wide variety of vectors can be employed for this purpose and are known and available to those of ordinary skill in the art. See, e.g., Ausubel et al., 1987-1993. Factors of importance in selecting a particular plasmid or viral vector include: the ease with which recipient cells that contain the vector may be recognized and selected from those recipient cells which do not contain the vector; the number of copies of the vector which are desired in a particular host; and whether it is desirable to be able to “shuttle” the vector between host cells of different species.

Exemplary prokaryotic vectors known in the art include plasmids such as those capable of replication in E. coli. Other gene expression elements useful for the expression of DNA encoding SLITRK6 antibodies or antigen-binding portions thereof or other binding agents include, but are not limited to (a) viral transcription promoters and their enhancer elements, such as the SV40 early promoter. (Okayama et al., 3 Mol. Cell. Biol. 280 (1983)), Rous sarcoma virus LTR (Gorman et al., 79 PNAS 6777 (1982)), and Moloney murine leukemia virus LTR (Grosschedl et al., 41 Cell 885 (1985)); (b) splice regions and polyadenylation sites such as those derived from the SV40 late region (Okayarea et al., 1983), and (c) polyadenylation sites such as in SV40 (Okayama et al., 1983). Immunoglobulin-encoding DNA genes can be expressed as described by Liu et al., infra, and Weidle et al., 51 Gene 21 (1987), using as expression elements the SV40 early promoter and its enhancer, the mouse immunoglobulin H chain promoter enhancers, SV40 late region mRNA splicing, rabbit S-globin intervening sequence, immunoglobulin and rabbit S-globin polyadenylation sites, and SV40 polyadenylation elements.

For immunoglobulin encoding nucleotide sequences, the transcriptional promoter can be, for example, human cytomegalovirus, the promoter enhancers can be cytomegalovirus and mouse/human immunoglobulin.

In some embodiments, for expression of DNA coding regions in rodent cells, the transcriptional promoter can be a viral LTR sequence, the transcriptional promoter enhancers can be either or both the mouse immunoglobulin heavy chain enhancer and the viral LTR enhancer, and the polyadenylation and transcription termination regions. In other embodiments, DNA sequences encoding other proteins are combined with the above-recited expression elements to achieve expression of the proteins in mammalian cells.

Each coding region or gene fusion is assembled in, or inserted into, an expression vector. Recipient cells capable of expressing the SLITRK6 variable region(s) or antigen binding portions thereof or other binding agents are then transfected singly with nucleotides encoding a SLITRK6 antibody or an antibody polypeptide or antigen-binding portion thereof or other binding agent, or are co-transfected with a polynucleotide(s) encoding VH and VL chain coding regions or other binding agents. The transfected recipient cells are cultured under conditions that permit expression of the incorporated coding regions and the expressed antibody chains or intact antibodies or antigen binding portions or other binding agents are recovered from the culture.

In some embodiments, the nucleic acids containing the coding regions encoding a SLITRK6 antibody or antigen-binding portion thereof or other binding agent are assembled in separate expression vectors that are then used to co-transfect a recipient host cell. Each vector can contain one or more selectable genes. For example, in some embodiments, two selectable genes are used, a first selectable gene designed for selection in a bacterial system and a second selectable gene designed for selection in a eukaryotic system, wherein each vector has a set of coding regions. This strategy results in vectors which first direct the production, and permit amplification, of the nucleotide sequences in a bacterial system. The DNA vectors so produced and amplified in a bacterial host are subsequently used to co-transfect a eukaryotic cell, and allow selection of a co-transfected cell carrying the desired transfected nucleic acids (e.g., containing SLITRK6 antibody heavy and light chains). Non-limiting examples of selectable genes for use in a bacterial system are the gene that confers resistance to ampicillin and the gene that confers resistance to chloramphenicol. Selectable genes for use in eukaryotic transfectants include the xanthine guanine phosphoribosyl transferase gene (designated gpt) and the phosphotransferase gene from Tn5 (designated neo). Alternatively the fused nucleotide sequences encoding VH and VL chains can be assembled on the same expression vector.

For transfection of the expression vectors and production of the SLITRK6 antibodies or antigen binding portions thereof or other binding agents, the recipient cell line can be a Chinese Hamster ovary cell line (e.g., DG44) or a myeloma cell. Myeloma cells can synthesize, assemble and secrete immunoglobulins encoded by transfected immunoglobulin genes and possess the mechanism for glycosylation of the immunoglobulin. For example, in some embodiments, the recipient cell is the recombinant Ig-producing myeloma cell SP2/0. SP2/0 cells only produce immunoglobulins encoded by the transfected genes. Myeloma cells can be grown in culture or in the peritoneal cavity of a mouse, where secreted immunoglobulin can be obtained from ascites fluid.

An expression vector encoding a SLITRK6 antibody or antigen-binding portion thereof or other binding agent can be introduced into an appropriate host cell by any of a variety of suitable means, including such biochemical means as transformation, transfection, protoplast fusion, calcium phosphate-precipitation, and application with polycations such as diethylaminoethyl (DEAE) dextran, and such mechanical means as electroporation, direct microinjection and microprojectile bombardment. Johnston et al., 240 Science 1538 (1988), as known to one of ordinary skill in the art.

Yeast provides certain advantages over bacteria for the production of immunoglobulin heavy and light chains. Yeasts carry out post-translational peptide modifications including glycosylation. A number of recombinant DNA strategies exist that utilize strong promoter sequences and high copy number plasmids which can be used for production of the desired proteins in yeast. Yeast recognizes leader sequences of cloned mammalian gene products and secretes polypeptides bearing leader sequences (i.e., pre-polypeptides). See, e.g., Hitzman et al., 11th Intl. Conf. Yeast, Genetics & Molec. Biol. (Montpelier, France, 1982).

Yeast gene expression systems can be routinely evaluated for the levels of production, secretion and the stability of antibodies, and assembled SLITRK6 antibodies and antigen binding portions thereof and other binding agents. Various yeast gene expression systems incorporating promoter and termination elements from the actively expressed genes coding for glycolytic enzymes produced in large quantities when yeasts are grown in media rich in glucose can be utilized. Known glycolytic genes can also provide very efficient transcription control signals. For example, the promoter and terminator signals of the phosphoglycerate kinase (PGK) gene can be utilized. Another example is the translational elongation factor 1alpha promoter, such as that from Chinese hamster cells. A number of approaches can be taken for evaluating optimal expression plasmids for the expression of immunoglobulins in yeast. See II DNA Cloning 45, (Glover, ed., IRL Press, 1985) and e.g., U.S. Publication No. US 2006/0270045 A1.

Bacterial strains can also be utilized as hosts for the production of the antibody molecules or antigen binding portions thereof or other binding agents as described herein. E. coli K12 strains such as E. coli W3110, Bacillus species, enterobacteria such as Salmonella typhimurium or Serratia marcescens, and various Pseudomonas species can be used. Plasmid vectors containing replicon and control sequences that are derived from species compatible with a host cell are used in connection with these bacterial hosts. The vector carries a replication site, as well as specific genes which are capable of providing phenotypic selection in transformed cells. A number of approaches can be taken for evaluating the expression plasmids for the production of SLITRK6 antibodies and antigen binding portions thereof and other binding agents in bacteria (see Glover, 1985; Ausubel, 1987, 1993; Sambrook, 1989; Colligan, 1992-1996).

Host mammalian cells can be grown in vitro or in vivo. Mammalian cells provide post-translational modifications to immunoglobulin molecules including leader peptide removal, folding and assembly of VH and VL chains, glycosylation of the antibody molecules, and secretion of functional antibody and/or antigen binding portions thereof or other binding agents.

Mammalian cells which can be useful as hosts for the production of antibody proteins, in addition to the cells of lymphoid origin described above, include cells of fibroblast origin, such as Vero or CHO-K1 cells. Exemplary eukaryotic cells that can be used to express immunoglobulin polypeptides include, but are not limited to, COS cells, including COS 7 cells; 293 cells, including 293-6E cells; CHO cells, including CHO—S and DG44 cells; PERC6TM cells (Crucell); and NSO cells. In some embodiments, a particular eukaryotic host cell is selected based on its ability to make desired post-translational modifications to the heavy chains and/or light chains. For example, in some embodiments, CHO cells produce polypeptides that have a higher level of sialylation than the same polypeptide produced in 293 cells.

In some embodiments, one or more SLITRK6 antibodies or antigen-binding portions thereof or other binding agents can be produced in vivo in an animal that has been engineered or transfected with one or more nucleic acid molecules encoding the polypeptides, according to any suitable method.

In some embodiments, an antibody or antigen-binding portion thereof is produced in a cell-free system. Non-limiting exemplary cell-free systems are described, e.g., in Sitaraman et al., Methods Mol. Biol. 498: 229-44 (2009); Spirin, Trends Biotechnol. 22: 538-45 (2004); and Endo et al., Biotechnol. Adv. 21: 695-713 (2003).

Many vector systems are available for the expression of the VH and VL chains in mammalian cells (see Glover, 1985). Various approaches can be followed to obtain intact antibodies. As discussed above, it is possible to co-express VH and VL chains and optionally the associated constant regions in the same cells to achieve intracellular association and linkage of VH and VL chains into complete tetrameric H2L2 antibodies or antigen-binding portions thereof. The co-expression can occur by using either the same or different plasmids in the same host. Nucleic acids encoding the VH and VL chains or antigen binding portions thereof can be placed into the same plasmid, which is then transfected into cells, thereby selecting directly for cells that express both chains. Alternatively, cells can be transfected first with a plasmid encoding one chain, for example the VL chain, followed by transfection of the resulting cell line with a VH chain plasmid containing a second selectable marker. Cell lines producing antibodies, antigen-binding portions thereof via either route could be transfected with plasmids encoding additional copies of peptides, VH, VL, or VH plus VL chains in conjunction with additional selectable markers to generate cell lines with enhanced properties, such as higher production of assembled SLITRK6 antibodies or antigen binding portions thereof or other binding agents or enhanced stability of the transfected cell lines.

Additionally, plants have emerged as a convenient, safe and economical alternative expression system for recombinant antibody production, which are based on large scale culture of microbes or animal cells. SLITRK6 binding antibodies or antigen binding portions thereof or other binding agents can be expressed in plant cell culture, or plants grown conventionally. The expression in plants may be systemic, limited to sub-cellular plastids, or limited to seeds (endosperms). See, e.g., U.S. Patent Pub. No. 2003/0167531; U.S. Pat. Nos. 6,080,560; 6,512,162; and WO 0129242. Several plant-derived antibodies have reached advanced stages of development, including clinical trials (see, e.g., Biolex, N.C.).

For intact antibodies, the variable regions (VH and VL regions) of the SLITRK6 antibodies are typically linked to at least a portion of an immunoglobulin constant region (Fc) or domain, typically that of a human immunoglobulin. Human constant region DNA sequences can be isolated in accordance with well-known procedures from a variety of human cells, such as immortalized B-cells (WO 87/02671). A SLITRK6 binding antibody can contain both light chain and heavy chain constant regions. The heavy chain constant region can include CH1, hinge, CH2, CH3, and, optionally, CH4 regions. In some embodiments, the CH2 domain can be deleted or omitted.

Techniques described for the production of single chain antibodies (see, e.g. U.S. Pat. No. 4,946,778; Bird, Science 242:423-42 (1988); Huston et al., Proc. Natl. Acad. Sci. USA 85:5879-5883 (1988); and Ward et al., Nature 334:544-54 (1989); which are incorporated by reference herein in their entireties) can be adapted to produce single chain antibodies that specifically bind to SLITRK6. Single chain antibodies are formed by linking the heavy and light chain variable regions of the Fv region via an amino acid bridge, resulting in a single chain polypeptide. Techniques for the assembly of functional Fv portions in E. coli can also be used (see, e.g. Skerra et al., Science 242:1038-1041 (1988); which is incorporated by reference herein in its entirety).

In some embodiments, an antigen binding portion or other binding agent comprises one or more scFvs. An scFv can be, for example, a fusion protein of the variable regions of the heavy (VH) and light chain (VL) variable regions of an antibody, connected with a short linker peptide of ten to about 25 amino acids. The linker is usually rich in glycine for flexibility, as well as serine or threonine for solubility, and can either connect the N-terminus of the VH with the C-terminus of the VL, or vice versa. This protein retains the specificity of the original antibody, despite removal of the constant regions and the introduction of the linker. scFv antibodies are, e.g. described in Houston, J. S., Methods in Enzymol. 203 (1991) 46-96. Methods for making scFv molecules and designing suitable peptide linkers are described in, for example, U.S. Pat. Nos. 4,704,692; 4,946,778; Raag and Whitlow, FASEB 9:73-80 (1995) and Bird and Walker, TIBTECH, 9: 132-137 (1991). scFv-Fcs have been described by Sokolowska-Wedzina et al., Mol. Cancer Res. 15(8):1040-1050, 2017.

In some embodiments, an antigen binding portion or other binding agent is a single-domain antibody is an antibody portion consisting of a single monomeric variable antibody domain. Single domains antibodies can be derived from the variable domain of the antibody heavy chain from camelids (e.g., nanobodies or VHH portions). Furthermore, a single-domain antibody can be an autonomous human heavy chain variable domain (aVH) or VNAR portions derived from sharks (see, e.g., Hasler et al., Mol. Immunol. 75:28-37, 2016).

Techniques for producing single domain antibodies (DABs or VHH) are known in the art, as disclosed for example in Cossins et al. (2006, Prot Express Purif 51:253-259) and Li et al. (Immunol. Lett. 188:89-95, 2017). Single domain antibodies may be obtained, for example, from camels, alpacas or llamas by standard immunization techniques. (See, e.g., Muyldermans et al., TIBS 26:230-235, 2001; Yau et al., J Immunol Methods 281:161-75, 2003; and Maass et al., J Immunol Methods 324:13-25, 2007.) A VHH may have potent antigen-binding capacity and can interact with epitopes that are inacessible to conventional VH-VL pairs (see, e.g., Muyldermans et al., 2001). Alpaca serum IgG contains about 50% camelid heavy chain only IgG antibodies (HCAbs) (see, e.g., Maass et al., 2007). Alpacas may be immunized with antigens and VHHs can be isolated that bind to and neutralize the target antigen (see, e.g., Maass et al., 2007). PCR primers that amplify alpaca VHH coding sequences have been identified and can be used to construct alpaca VHH phage display libraries, which can be used for antibody fragment isolation by standard biopanning techniques well known in the art (see, e.g., Maass et al., 2007).

Techniques for making multispecific antibodies include, but are not limited to, recombinant co-expression of two immunoglobulin heavy chain-light chain pairs having different specificities (see, e.g., Milstein and Cuello, Nature 305: 537 (1983)), WO 93/08829, and Traunecker et al., EMBO J. 10: 3655 (1991)), and “knob-in-hole” engineering (see, e.g., U.S. Pat. No. 5,731,168; Carter (2001), J Immunol Methods 248, 7-15). Multi-specific antibodies may also be made by engineering electrostatic steering effects for making antibody Fc-heterodimeric molecules (see, e.g., WO 2009/089004A1); cross-linking of two or more antibodies or antigen binding portions thereof (see, e.g., U.S. Pat. No. 4,676,980, and Brennan et al., Science, 229: 81 (1985)); using leucine zippers to produce bi-specific antibodies (see, e.g., Kostelny et al., J. Immunol., 148(5):1547-1553 (1992)); using “diabody” technology for making bispecific antibody portions (see, e.g., Hollinger et al., Proc. Natl. Acad. Sci. USA, 90:6444-6448 (1993)); and using single-chain Fv (scFv) dimers (see, e.g. Gruber et al., J. Immunol., 152:5368 (1994)); and preparing trispecific antibodies as described, e.g., in Tutt et al. J. Immunol. 147: 60 (1991).

Engineered antibodies with three or more functional antigen binding sites, including “Octopus antibodies,” also can be binding agents (see, e.g. US 2006/0025576A1).

The binding agents (e.g., antibodies or antigen binding portions) herein also include a “Dual Acting FAb” or “DAF” comprising an antigen binding site that binds to two different antigens (see, e.g., US 2008/0069820 and Bostrom et al., 2009, Science 323:1610-14). “Crossmab” antibodies are also included herein (see e.g. WO 2009/080251, WO 2009/080252, WO2009/080253, WO2009/080254, and WO2013/026833).

In some embodiments, the binding agents comprise different antigen-binding sites, fused to one or the other of the two subunits of the Fc domain; thus, the two subunits of the Fc domain may be comprised in two non-identical polypeptide chains. Recombinant co-expression of these polypeptides and subsequent dimerization leads to several possible combinations of the two polypeptides. To improve the yield and purity of the bispecific molecules in recombinant production, it will thus be advantageous to introduce in the Fc domain of the binding agent a modification promoting the association of the desired polypeptides.

Generally, this method involves replacement of one or more amino acid residues at the interface of the two Fc domains by charged amino acid residues so that homodimer formation becomes electrostatically unfavorable but heterodimerization electrostatically favorable.

In some embodiments, a binding agent is a “bispecific T cell engager” or BiTE (see, e.g., WO2004/106381, WO2005/061547, WO2007/042261, and WO2008/119567). This approach utilizes two antibody variable domains arranged on a single polypeptide. For example, a single polypeptide chain can include two single chain Fv (scFv) portions, each having a variable heavy chain (VH) and a variable light chain (VL) domain separated by a polypeptide linker of a length sufficient to allow intramolecular association between the two domains. This single polypeptide further includes a polypeptide spacer sequence between the two scFvs. Each scFv recognizes a different epitope, and these epitopes may be specific for different proteins, such that both proteins are bound by the BiTE.

As it is a single polypeptide, the bispecific T cell engager may be expressed using any prokaryotic or eukaryotic cell expression system known in the art, e.g., a CHO cell line. However, specific purification techniques (see, e.g., EP1691833) may be necessary to separate monomeric bispecific T cell engagers from other multimeric species, which may have biological activities other than the intended activity of the monomer. In one exemplary purification scheme, a solution containing secreted polypeptides is first subjected to a metal affinity chromatography, and polypeptides are eluted with a gradient of imidazole concentrations. This eluate is further purified using anion exchange chromatography, and polypeptides are eluted using with a gradient of sodium chloride concentrations. Finally, this eluate is subjected to size exclusion chromatography to separate monomers from multimeric species. In some embodiments, a binding agent that is a bispecific antibody is composed of a single polypeptide chain comprising two single chain FV portions (scFV) fused to each other by a peptide linker.

In some embodiments, a binding agent is multispecific, such as an IgG-scFV. IgG-scFv formats include IgG(H)-scFv, scFv-(H)IgG, IgG(L)-scFv, svFc-(L)IgG, 2scFV-IgG and IgG-2scFv. These and other bispecific antibody formats and methods of making them have been described in for example, Brinkmann and Kontermann, MAbs 9(2):182-212 (2017); Wang et al., Antibodies, 2019, 8, 43; Dong et al., 2011, MAbs 3:273-88; Natsume et al., J. Biochem. 140(3):359-368, 2006; Cheal et al., Mol. Cancer Ther. 13(7):1803-1812, 2014; and Bates and Power, Antibodies, 2019, 8, 28.

IgG-like dual-variable domain antibodies (DVD-Ig) have been described by Wu et al., 2007, Nat Biotechnol 25:1290-97; Hasler et al., Mol. Immunol. 75:28-37, 2016 and in WO 08/024188 and WO 07/024715. Triomabs have been described by Chelius et al., MAbs 2(3):309-319, 2010. 2-in-1-IgGs have been described by Kontermann et al., Drug Discovery Today 20(7):838-847, 2015. Tanden antibody or TandAb have been described by Kontermann et al., id. ScFv-HSA-scFv antibodies have also been described by Kontermann et al. (id.).

Intact (e.g., whole) antibodies, their dimers, individual light and heavy chains, or antigen binding portions thereof and other binding agents can be recovered and purified by known techniques, e.g., immunoadsorption or immunoaffinity chromatography, chromatographic methods such as HPLC (high performance liquid chromatography), ammonium sulfate precipitation, gel electrophoresis, or any combination of these. See generally, Scopes, Protein Purification (Springer-Verlag, N.Y., 1982). Substantially pure SLITRK6 binding antibodies or antigen binding portions thereof or other binding agents of at least about 90% to 95% homogeneity are advantageous, as are those with 98% to 99% or more homogeneity, particularly for pharmaceutical uses. Once purified, partially or to homogeneity as desired, an intact SLITRK6 antibody or antigen binding portions thereof or other binding agent can then be used therapeutically or in developing and performing assay procedures, immunofluorescent staining, and the like. See generally, Vols. I & II Immunol. Meth. (Lefkovits & Pernis, eds., Acad. Press, NY, 1979 and 1981).

### Antibody Drug Conjugates

In some embodiments, a SLITRK6 antibody, antigen binding portion or other binding agent as described herein is part of a SLITRK6 antibody drug conjugate (also referred to as a SLITRK6 conjugate or SLITRK6 ADC). In some embodiments, the SLITRK6 antibody, antigen binding portion or other binding agent is attached to at least one linker, and at least one drug is attached to each linker. As used herein, in the context of a conjugate, the term “drug” refers to cytotoxic agents (such as chemotherapeutic agents or drugs), immunomodulatory agents, nucleic acids (including siRNAs), growth inhibitory agents, toxins (e.g., protein toxins, enzymatically active toxins of bacterial, fungal, plant, or animal origin, or fragments thereof), radioactive isotopes, PROTACs and other compounds that are active against target cells when delivered to those cells.

### Cytotoxic Agents

In some embodiments, a SLITRK6 conjugate includes at least one drug (or termed as “drug unit”) that is cytotoxic agent. A “cytotoxic agent” refers to an agent that has a cytotoxic effect on a cell. A “cytotoxic effect” refers to the depletion, elimination and/or the killing of a target cell(s). Cytotoxic agents include, for example, tubulin disrupting agents, topoisomerase inhibitors, DNA minor groove binders, and DNA alkylating agents.

Tubulin disrupting agents include, for example, auristatins, dolastatins, tubulysins, colchicines, vinca alkaloids, taxanes, cryptophycins, maytansinoids, hemiasterlins, as well as other tubulin disrupting agents. Auristatins are derivatives of the natural product dolastatin 10. Exemplary auristatins include MMAE (N-methylvaline-valine-dolaisoleuine-dolaproine-norephedrine), MMAF (N-methylvaline-valine-dolaisoleuine-dolaproine-phenylalanine) and AFP (see WO2004/010957 and WO2007/008603). Other auristatin like compounds are disclosed in, for example, Published US Application Nos. US2021/0008099, US2017/0121282, US2013/0309192 and US2013/0157960. Dolastatins include, for example, dolastatin 10 and dolastatin 15 (see, e.g., Pettit et al., J. Am. Chem. Soc., 1987, 109, 6883-6885; Pettit et al., Anti-Cancer Drug Des., 1998, 13, 243-277; and Published US Application US2001/0018422). Additional dolastatin derivatives contemplated for use herein are disclosed in U.S. Pat. No. 9,345,785, incorporated herein by reference. In some embodiments, the tubulin disrupting agent is MMAE.

Tubulysins include, but are not limited to, tubulysin D, tubulysin M, tubuphenylalanine and tubutyrosine. WO2017/096311 and WO/2016-040684 describe tubulysin analogs including tubulysin M.

Colchicines include, but are not limited to, colchicine and CA-4.

Vinca alkaloids include, but are not limited to, vinblastine (VBL), vinorelbine (VRL), vincristine (VCR) and vindesine (VOS).

Taxanes include, but are not limited to, paclitaxel and docetaxel.

Cryptophycins include but are not limited to cryptophycin-1 and cryptophycin-52.

Maytansinoids include, but are not limited to, maytansine, maytansinol, maytansine analogs in DM1, DM3 and DM4, and ansamatocin-2. Exemplary maytansinoid drug moieties include those having a modified aromatic ring, such as: C-19-dechloro (U.S. Pat. No. 4,256,746) (prepared by lithium aluminum hydride reduction of ansamitocin P2); C-20-hydroxy (or C-20-demethyl)+/−C-19-dechloro (U.S. Pat. Nos. 4,361,650 and 4,307,016) (prepared by demethylation using Streptomyces or Actinomyces or dechlorination using LAH); and C-20-demethoxy, C-20-acyloxy (—OCOR), +/−dechloro (U.S. Pat. No. 4,294,757) (prepared by acylation using acyl chlorides), and those having modifications at other positions.

Maytansinoid drug moieties also include those having modifications such as: C-9-SH (U.S. Pat. No. 4,424,219) (prepared by the reaction of maytansinol with H2S or P2S5); C-14-alkoxymethyl(demethoxy/CH2OR) (U.S. Pat. No. 4,331,598); C-14-hydroxymethyl or acyloxymethyl (CH2OH or CH2OAc) (U.S. Pat. No. 4,450,254) (prepared from Nocardia); C-15-hydroxy/acyloxy (U.S. Pat. No. 4,364,866) (prepared by the conversion of maytansinol by Streptomyces); C-15-methoxy (U.S. Pat. Nos. 4,313,946 and 4,315,929) (isolated from Trewia nudiflora); C-18-N-demethyl (U.S. Pat. Nos. 4,362,663 and 4,322,348) (prepared by the demethylation of maytansinol by Streptomyces); and 4,5-deoxy (U.S. Pat. No. 4,371,533) (prepared by the titanium trichloride/LAH reduction of maytansinol).

Hemiasterlins include but are not limited to, hemiasterlin and HTI-286.

Other tubulin disrupting agents include taccalonolide A, taccalonolide B, taccalonolide AF, taccalonolide AJ, taccalonolide Al-epoxide, discodermolide, epothilone A, epothilone B, and laulimalide.

In some embodiments, a cytotoxic agent can be a topoisomerase inhibitor, such as a camptothecin. Exemplary camptothecins include, for example, camptothecin, irinotecan (also referred to as CPT-11), belotecan, (7-(2-(N-isopropylamino)ethyl)camptothecin), topotecan, 10-hydroxy-CPT, SN-38, exatecan and the exatecan analog DXd (see US20150297748). Other camptothecins are disclosed in WO1996/021666, WO00/08033, US2016/0229862 and WO2020/156189.

In some embodiments, a cytotoxic agent is a duocarmcycin, including the synthetic analogues, KW-2189 and CBI-TMI.

### Immune Modulatory Agents

In some embodiments, a drug is an immune modulatory agent. An immune modulatory agent can be, for example, a TLR7 and/or TLR8 agonist, a STING agonist, a RIG-1 agonist or other immune modulatory agent.

In some embodiments, a drug is an immune modulatory agent, such as a TLR7 and/or TLR8 agonist. In some embodiments, a TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, and PolyG3. In some embodiments, the TLR7 agonist is selected from an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide or a benzonaphthyridine. In some embodiments, a TLR7 agonist is a non-naturally occurring compound. Examples of TLR7 modulators include GS-9620, GSK-2245035, imiquimod, resiquimod, DSR-6434, DSP-3025, IMO-4200, MCT-465, MEDI-9197, 3M-051, SB-9922, 3M-052, Limtop, TMX-30X, TMX-202, RG-7863, RG-7795, and the compounds disclosed in US20160168164 (Janssen), US 20150299194 (Roche), US20110098248 (Gilead Sciences), US20100143301 (Gilead Sciences), and US20090047249 (Gilead Sciences).

In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA. In some embodiments, a TLR8 agonist is selected from a benzazepine, an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, and a tetrahydropyridopyrimidine. In some embodiments, a TLR8 agonist is a non-naturally occurring compound. Examples of TLR8 agonists include motolimod, resiquimod, 3M-051, 3M-052, MCT-465, IMO-4200, VTX-763, VTX-1463.

In some embodiments, a TLR8 agonist can be any of the compounds described WO2018/170179, WO2020/056198 and WO2020056194.

Other TLR7 and TLR8 agonists are disclosed in, for example, WO2016142250, WO2017046112, WO2007024612, WO2011022508, WO2011022509, WO2012045090, WO2012097173, WO2012097177, WO2017079283, US20160008374, US20160194350, US20160289229, U.S. Pat. No. 6,043,238, US20180086755 (Gilead), WO2017216054 (Roche), WO2017190669 (Shanghai De Novo Pharmatech), WO2017202704 (Roche), WO2017202703 (Roche), WO20170071944 (Gilead), US20140045849 (Janssen), US20140073642 (Janssen), WO2014056953 (Janssen), WO2014076221 (Janssen), WO2014128189 (Janssen), US20140350031 (Janssen), WO2014023813 (Janssen), US20080234251 (Array Biopharma), US20080306050 (Array Biopharma), US20100029585 (Ventirx Pharma), US20110092485 (Ventirx Pharma), US20110118235 (Ventirx Pharma), US20120082658 (Ventirx Pharma), US20120219615 (Ventirx Pharma), US20140066432 (Ventirx Pharma), US20140088085 (Ventirx Pharma), US20140275167 (Novira Therapeutics), and US20130251673 (Novira Therapeutics), WO2018198091(Novartis AG), and US20170131421 (Novartis AG).

In some embodiments, an immune modulatory agent is a STING agonist. Examples of STING agonists include, for example, those disclosed in WO2020059895, WO2015077354, WO2020227159, WO2020075790, WO2018200812, and WO2020074004.

In some embodiments, an immune modulatory agent is a RIG-1 agonist. Examples of RIG-1 agonists include KIN1148, SB-9200, KIN700, KIN600, KIN500, KIN100, KIN101, KIN400 and KIN2000.

### Toxins

In some embodiments, a drug is an enzymatically active toxin or fragment thereof, including but not limited to diphtheria A chain, nonbinding active fragments of diphtheria toxin, exotoxin A chain (from Pseudomonas aeruginosa), ricin A chain, abrin A chain, modeccin A chain, alpha-sarcin, Aleurites fordii proteins, dianthin proteins, Phytolaca americana proteins (PAPI, PAPII, and PAP-S), Momordica charantia inhibitor, curcin, crotin, Sapaonaria officinalis inhibitor, gelonin, mitogellin, restrictocin, phenomycin, enomycin, and the tricothecenes.

### Radioisotopes

In some embodiments, a drug is a radioactive atom. Avariety of radioactive isotopes are available for the production of radioconjugates. Examples include I131, I125, Y90, Re186, Re188, Sm153, Bi213, P32, Pb212 and radioactive isotopes of Lutetium (e.g., Lu177).

### PROTACs

In some embodiments, a drug is a proteolysis targeted chimera (PROTAC). PROTACs are described in, for example, Published US Application Nos. 20210015942, 20210015929, 20200392131, 20200216507, US20200199247 and US20190175612; the disclosures of which are incorporated by reference herein.

### Linkers

The SLITRK6 conjugates typically comprise at least one linker, each linker having at least one drug attached to it. Typically, a conjugate includes a linker between a SLITRK6 antibody (or antigen binding portion thereof or other binding agent) and the drug (in some cases termed “drug unit”). In various embodiments, a linker may be a protease cleavable linker, an acid-cleavable linker, a disulfide linker, a disulfide-containing linker, or a disulfide-containing linker having a dimethyl group adjacent the disulfide bond (e.g., an SPDB linker) (see, e.g., Jain et al., Pharm. Res. 32:3526-3540 (2015); Chari et al., Cancer Res. 52:127-131 (1992); U.S. Pat. No. 5,208,020), a self-stabilizing linker (see, e.g., WO2018/031690 and WO2015/095755 and Jain et al., Pharm. Res. 32:3526-3540 (2015)), a non-cleavable linker (see, e.g., WO2007/008603), a photolabile linker, and/or a hydrophilic linker (see, e.g., WO2015/123679).

In some embodiments, a linker is a cleavable linker that is cleavable under intracellular conditions, such that cleavage of the linker releases the drug from the antibody (or antigen binding portion thereof or other binding agent) and/or linker in the intracellular environment. For example, in some embodiments, a linker is cleavable by a cleaving agent that is present in the intracellular environment (e.g., within a lysosome or endosome or caveolae). A linker can be, for example, a peptidyl linker that is cleaved by an intracellular peptidase or protease enzyme, including, but not limited to, a lysosomal or endosomal protease (see, e.g., WO2004/010957, US20150297748, US2008/0166363, US20120328564 and US20200347075). Typically, a peptidyl linker is at least one amino acid long or at least two amino acids long. Intracellular cleaving agents can include cathepsins B and D and plasmin, all of which are known to hydrolyze dipeptide drug derivatives resulting in the release of active drug inside target cells (see, e.g., Dubowchik and Walker, 1999, Pharm. Therapeutics 83:67-123). Most typical are peptidyl linkers that are cleavable by enzymes that are present in target antigen-expressing cells. For example, a peptidyl linker that is cleavable by the thiol-dependent protease cathepsin-B, which is highly expressed in cancerous tissue, can be used (e.g., a Phe-Leu or a Gly-Phe-Leu-Gly linker). Other such linkers are described, for example, in U.S. Pat. No. 6,214,345. In specific embodiments, the peptidyl linker cleavable by an intracellular protease is a Val-Cit linker or a Phe-Lys linker (see, e.g., U.S. Pat. No. 6,214,345, which describes the synthesis of doxorubicin with the val-cit linker) or Gly-Gly-Phe-Gly (SEQ ID NO: 57) linker (see, e.g., US2015/0297748). One advantage of using intracellular proteolytic release of the drug is that the drug is typically attenuated when conjugated and the serum stabilities of the conjugates are typically high. See also U.S. Pat. No. 9,345,785.

As used herein, the terms “intracellularly cleaved” and “intracellular cleavage” refer to a metabolic process or reaction inside a cell on an antibody drug conjugate, whereby the covalent attachment, e.g., the linker, between a drug (e.g., a cytotoxic agent) and the antibody is broken, resulting in the free drug, or other metabolite of the conjugate dissociated from the antibody inside the cell. The cleaved moieties of the conjugate are thus intracellular metabolites.

In some embodiments, a cleavable linker is pH-sensitive, i.e., sensitive to hydrolysis at certain pH values. Typically, a pH-sensitive linker is hydrolyzable under acidic conditions. For example, an acid-labile linker that is hydrolyzable in the lysosome (e.g., a hydrazone, semicarbazone, thiosemicarbazone, cis-aconitic amide, orthoester, acetal, ketal, or the like) can be used. (See, e.g., U.S. Pat. Nos. 5,122,368; 5,824,805; and 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, a hydrolyzable linker is a thioether linker (such as, for example, a thioether attached to the drug via an acylhydrazone bond (see, e.g., U.S. Pat. No. 5,622,929)).

In some embodiments, a linker is cleavable under reducing conditions (e.g., a disulfide linker). A variety of disulfide linkers are known, including, for example, those that can be formed using SATA (N-succinimidyl-5-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT (see, e.g., Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987. See also U.S. Pat. No. 4,880,935.)

In some embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12). In some embodiments, the linker is not cleavable, such as a maleimidocaproyl linker, and the drug is released by antibody degradation. (See U.S. Publication No. 2005/0238649).

In some embodiments, a linker is not substantially sensitive to the extracellular environment. As used herein, “not substantially sensitive to the extracellular environment,” in the context of a linker, means that no more than about 20%, typically no more than about 15%, more typically no more than about 10%, and even more typically no more than about 5%, no more than about 3%, or no more than about 1% of the linkers, in a sample of the antibody drug conjugate (ADC), are cleaved when the ADC is present in an extracellular environment (e.g., in plasma). Whether a linker is not substantially sensitive to the extracellular environment can be determined, for example, by incubating independently with plasma both (a) the ADC (the “ADC sample”) and (b) an equal molar amount of unconjugated antibody or drug (the “control sample”) for a predetermined time period (e.g., 2, 4, 8, 16, or 24 hours) and then comparing the amount of unconjugated antibody or drug present in the ADC sample with that present in control sample, as measured, for example, by high performance liquid chromatography.

In some embodiments, a linker promotes cellular internalization. In some embodiments, a linker promotes cellular internalization when conjugated to the drug such as a cytotoxic agent (i.e., in the milieu of the linker-drug moiety of the ADC as described herein). In yet other embodiments, a linker promotes cellular internalization when conjugated to both the drug and the SLITRK6 antibody (i.e., in the milieu of the ADC as described herein).

A variety of linkers that can be used with the present compositions and methods are described in WO 2004010957. In some embodiments, a protease cleavable linker comprises a thiol-reactive spacer and a dipeptide. In some embodiments, the protease cleavable linker consists of a thiol-reactive maleimidocaproyl spacer, a valine-citrulline dipeptide, and a p-amino-benzyloxycarbonyl spacer.

In some embodiments, an acid cleavable linker is a hydrazine linker or a quaternary ammonium linker (see WO2017/096311 and WO2016/040684.)

In some embodiments, a linker is a self-stabilizing linker comprising a maleimide group as described in U.S. Pat. No. 9,504,756.

In some embodiments, a linker is a hydrophilic linker, such as, for example, the hydrophilic peptides in WO2015/123679 and the sugar alcohol polymer-based linkers disclosed in WO2013/012961 and WO2019/213046 (each of which is incorporated by reference in its entirety).

In some embodiments, the binging agents herein disclosed may be connected to a linker as disclosed in WO2023280227, the contents of which are incorporated by reference in its entirety.

In other embodiments, conjugates of a SLITRK6 antibody (or antigen binding portion or other binding agent) and a drug may be made using a variety of bifunctional protein coupling agents such as N-succinimidyl-3-(2-pyridyldithio) propionate (SPDP), succinimidyl-4-(N-maleimidomethyl) cyclohexane-1-carboxylate (SMCC), iminothiolane (IT), bifunctional derivatives of imidoesters (such as dimethyl adipimidate HCl), active esters (such as disuccinimidyl suberate), aldehydes (such as glutaraldehyde), bis-azido compounds (such as bis (p-azidobenzoyl) hexanediamine), bis-diazonium derivatives (such as bis-(p-diazoniumbenzoyl)-ethylenediamine), diisocyanates (such as toluene 2,6-diisocyanate), and bis-active fluorine compounds (such as 1,5-difluoro-2,4-dinitrobenzene). Chelating agents for conjugation of a radionucleotide(s) to an antibody, antigen binding portion thereof or other binding agent have been described in, for example WO94/11026.

The conjugates of a SLITRK6 antibodies (or antigen binding portion or other binding agent) include, but are not limited to such conjugates prepared with cross-linker reagents including, but not limited to, BMPS, EMCS, GMBS, HBVS, LC-SMCC, MBS, MPBH, SBAP, SIA, SIAB, SMCC, SMPB, SMPH, sulfo-EMCS, sulfo-GMBS, sulfo-KMUS, sulfo-MBS, sulfo-SIAB, sulfo-SMCC, and sulfo-SMPB, and SVSB (succinimidyl-(4-vinylsulfone)benzoate) which are commercially available (e.g., from Pierce Biotechnology, Inc., Rockford, IL., U.S.A).

In some embodiments, a linker is attached to a terminus of an amino acid sequence of an antibody, antigen binding portion or other binding agent or can be attached to a side chain modification of an antibody, antigen binding portion or other binding agent, such as the side chain of a lysine, serine, threonine, cysteine, tyrosine, aspartic acid, a non-natural amino acid residue, glutamine, or glutamic acid residue. An attachment between an antibody, antigen binding portion or other binding agent and a linker or drug can be via any of a number of bonds, for example but not limited to, an amide bond, an ester bond, an ether bond, a carbon-nitrogen bond, a carbon-carbon single double or triple bond, a disulfide bond, or a thioether bond. Functional groups that can form such bonds include, for example, amino groups, carboxyl groups, aldehyde groups, azide groups, alkyne and alkene groups, ketones, carbonates, carbonyl functionalities bonded to leaving groups such as cyano and succinimidyl and hydroxyl groups.

In some embodiments, a linker is attached to an antibody, antigen binding portion or other binding agent at an interchain disulfide. In some embodiments, a linker is connected to an antibody, antigen binding portion or other binding agent at a hinge cysteine residue. In some embodiments, a linker is attached to an antibody, antigen binding portion or other binding agent at an engineered cysteine residue. In some embodiments, a linker is connected to an antibody, antigen binding portion or other binding agent at a lysine residue. In some embodiments, a linker is connected to an antibody, antigen binding portion or other binding agent at an engineered glutamine residue. In some embodiments, a linker is connected to an antibody, antigen binding portion or other binding agent at an unnatural amino acid engineered into the heavy chain.

In some embodiments, a linker is attached to an antibody, antigen binding portion or other binding agent via a sulfhydryl group. In some embodiments, a linker is attached to an antibody, antigen binding portion or other binding agent via a primary amine. In some embodiments, a linker is attached via a link created between an unnatural amino acid on an antibody, antigen binding portion or other binding agent by reacting with oxime bond that was formed by modifying a ketone group with an alkoxyamine on a drug.

In some embodiments, a linker is attached to an antibody, antigen binding portion or other binding agent via Sortase A linker. A Sortase A linker can be created by a Sortase A enzyme fusing an LPXTG recognition motif (SEQ ID NO: 58) to an N-terminal GGG motif to regenerate a native amide bond.

In some embodiments, the conjugate of the present disclosure comprises: one of the SLITRK6 antibodies, antigen binding portions thereof, and other binding agents, at least one linker attached to the binding agent; at least one drug unit, wherein each drug unit is attached to a linker, and wherein the linker optionally comprises at least one polar group.

In some embodiments, in the conjugate of the present disclosure, the linker is derived from a linker compound, or a stereoisomer or salt thereof, and the linker compound comprises: a linker unit; a stretcher group connected to the linker unit, an optional amino acid unit; and the at least one polar group; wherein: the stretcher group has an attachment site to the binding agent and an attachment site to the amino acid unit (when present) or the linker subunit; the amino acid unit (when present) has an attachment site to the stretcher group and an attachment site to the linker unit; and the linker unit has an attachment site to the amino acid unit (when present) or to the stretcher group and to the at least one drug unit.

Some of the components and variations of the linker (and the linker compound) are exemplified and demonstrated by the “linker embodiments” herein provided.

### ENUMERATED EMBODIMENTS

The linker (and linker compound) of the present disclosure is further illustrated by the following embodiments which should not be construed as limiting.

Embodiment 1. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein the polymer unit comprises the formula:

˜-R⁰—(R³—R¹—[O—CH₂—CH₂]ₙ₀—R⁶—([O—CH₂—CH₂]ₙ₀—R²—R³—(NR⁴R⁵)ₙ₁)ₙ₂)ₙ₃ (Ia)
or a stereoisomer or salt thereof, wherein:
   R⁰ is a functional group for attachment to a subunit of the amino acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₋₈, triazolyl, —C₁-C₁₂ alkylene-triazolyl-, —N(polyhydroxyl group)-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is selected from:

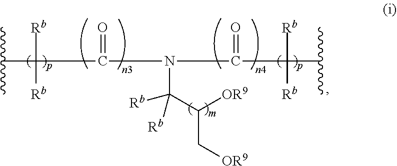

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4,
each v is independently 1-6, and
   n² is 1;

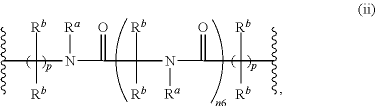

wherein:
   each R^{a} is independently H or C₁₋₆ alkyl,
   each R^{b} is independently H or C₁₋₆ alkyl,
   n⁶ is 1-10,
   each p is independently 0-6, and
n² is 1;

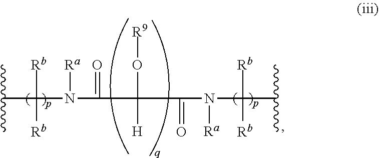

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   q is 1-8,
each v is independently 1-6, and
   n² is 1;

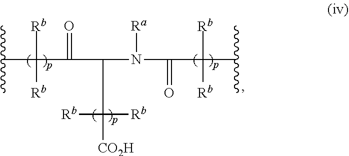

wherein:
   each R^{a} is independently H or C₁₋₆ alkyl,
   each R^{b} is independently H or C₁₋₆ alkyl,
   each p is independently 0-6, and
n² is 1;
(v) —R¹⁰—[O—CH₂—CH₂]₁₋₈—R¹⁰—, wherein:
   each R^{b} is independently H or C₁₋₆ alkyl,
   each R¹⁰ is independently

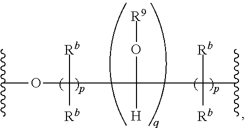

each p is independently 1-6,
each R⁹ is independently H, acetyl, —P(═O)(OH)₂, or —(CH₂)ᵥ—O—S(═O)₂(OH), and
q is 1-8;
n² is 1; and
(vi) —N—(R¹—X—R²—)₂, wherein:
   each X is independently —NR^{a}—C(O)— or —C(O)NR^{a}—, and
   n² is 2; and
      the wavy line (˜) indicates the attachment site of the amino acid unit to R⁰;
      each n^{O} is independently 2-26;
      each n¹ is independently 1-6; and
      n³ is 1-6.

Embodiment 2. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

˜R⁰—(R³—R¹—[O—CH₂—CH₂]ₙ₀—R²—(NR⁴R⁵)ₙ₁)ₙ₃ (Ia′)
or a stereoisomer or salt thereof, wherein:
   R⁰ is a functional group for attachment to a subunit of the amino acid unit;
   each R¹ and R² are independently a bond or C₁-C₆ alkylene;
   each R³ is independently —N(polyhydroxyl group)-, triazolyl, —C₁-C₁₂ alkylene-triazolyl-,

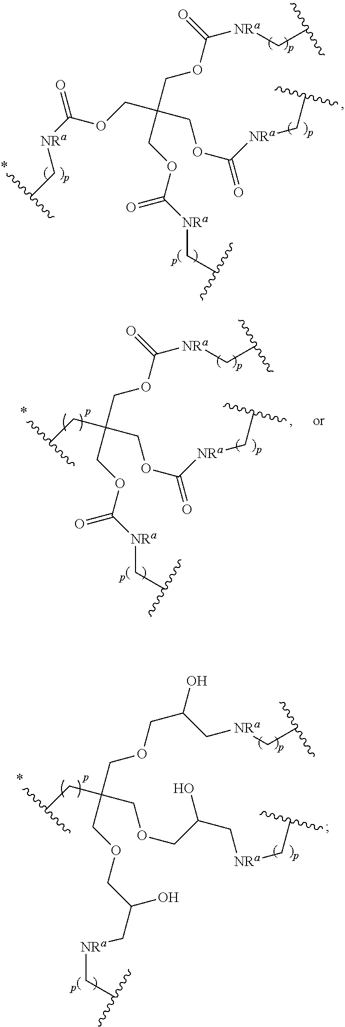

each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
each R^{a} is independently H or C₁₋₆ alkyl;

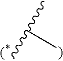

indicates the attachment site of R³ to R⁰
the wavy line,

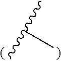

indicates the attachment site of the R³ to R¹;
each p is 1-6;
each n⁰ is independently 2-8;
each n¹ is independently 1-6; and
n³ is 1-6.

Embodiment 3. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises the formula:

˜R⁰—(R¹—[O—CH₂—CH₂]ₙ₀—R²—R³—(NR⁴R⁵)ₙ₁)ₙ₃ (Ia″)
or a stereoisomer or salt thereof, wherein:
   (i) R⁰ is a functional group for attachment to a subunit of the amino acid unit; each R¹ and R² are independently a bond or C₁-C₆ alkylene;
R³ is —C(O)—;
R⁴ is H;
R⁵ is independently a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate;
the wavy line (˜) indicates the attachment site of the amino acid unit to R⁰;
n⁰ is independently 2-26;
n¹ is 1-6; and
n³ is 1-6;
   (ii) R⁰ is —C(O)—;
R¹, R², and R³ are each a bond;
R⁴ and R⁵ are each independently H, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
n⁰ is 6;
n¹ is 1-6; and
n³ is 1;
   (iii) R⁰ is a functional group for attachment to a subunit of the amino acid unit;
R¹ and R² are each, independently, a bond or C₁-C₆ alkylene;
R³ is —NRₐ—C(O)—C₁-C₁₂ alkylene-C(O)—, wherein the alkylene is substituted with —SO₃H; R^{a} is H or C₁₋₆ alkyl;
R⁴ and R⁵ are each independently H, a carboxyl-containing moiety, a polyhydroxyl group, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
the wavy line (˜) indicates the attachment site of the amino acid unit to R⁰;
each n⁰ is independently 1-26;
n¹ is 1-6; and
n³ is 1-6; or
   (iv) R⁰ is

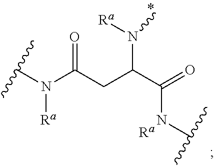

each R¹ is independently a bond or C₁-C₆ alkylene;
R² and R³ are each a bond;
R⁴ and R⁵ are each independently H, a polyhydroxyl group, a carboxyl-containing moiety, a substituted polyhydroxyl group, a —C(O)-polyhydroxyl group, a substituted —C(O)-polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R^{a} is independently H or C₁₋₆ alkyl;
the wavy line

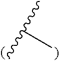

indicates the attachment site of R⁰ to the remainder of the polymer unit;
the wavy line (˜*) indicates the attachment site of the amino acid unit to R⁰;
n⁰ is 1-8;
n¹ is 1-6; and
n³ is 2.

Embodiment 4. A linker compound, or a stereoisomer or salt thereof, comprising:
(a) a linker unit having from 1 to 4 attachment sites for a drug unit, said linker unit comprising a moiety of formula:

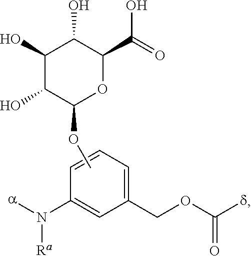

or a stereoisomer or salt thereof, wherein:
   α—represents a direct or indirect attachment site to an amino acid unit;
      δ— represents an attachment site to at least one of the drug units or for a linking group attached to the at least one of the drug units; and
R^{a} is H or C₁₋₆ alkyl;
(b) the amino acid unit having from 1 to 12 amino acid subunits; and
(c) at least one polar group attached to the amino acid unit, wherein the polar group comprises a polymer unit, optionally a sugar unit, and optionally a carboxyl unit.

Embodiment 5. A linker compound, or a stereoisomer or salt thereof, comprising:
(a) a linker unit having from 1 to 4 attachment sites for a drug unit;
(b) an amino acid unit having from 1 to 12 amino acid subunits; and
(c) at least one polar group attached to the amino acid unit, wherein the polar group comprises a polymer unit, optionally a sugar unit, and optionally a carboxyl unit, wherein said polymer unit comprises:
   (i) an optionally substituted polyamide comprising the formula

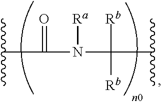

or a stereoisomer thereof, wherein each R^{a} is independently H or C₁₋₆ alkyl and each R^{b} is independently H or C₁₋₆ alkyl, and n⁰ is independently 2-26;
(ii) a substituted polyether comprising the formula

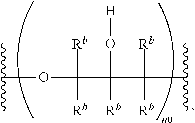

or a stereoisomer thereof, wherein each R^{b} is independently H or C₁₋₆ alkyl, and n^{o} is independently 2-26; or
(iii) combinations thereof.

Embodiment 6. The linker compound of Embodiment 4 or 5, wherein the at least one polar group attached to the amino acid unit comprises the formula:

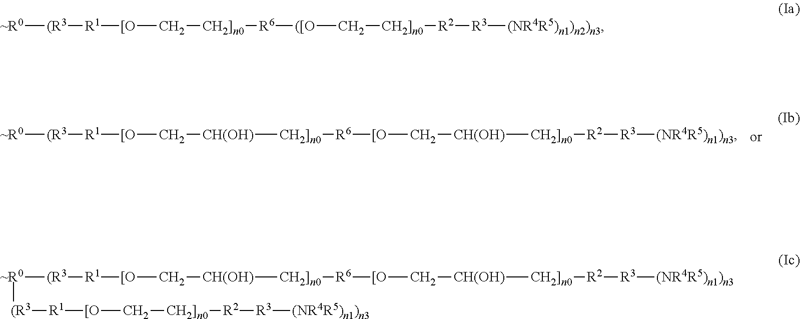

or a stereoisomer or salt thereof, wherein:
   R⁰ is a functional group for attachment to a subunit of the amino acid unit;
   each R¹ and R² are independently a bond or C₁-C₆ alkylene;
   each R³ is independently selected from a bond, C₁-C₁₂ alkylene, —C(O)—, —NR^{a}—C₁-C₁₂ alkylene, —C₁-C₁₂ alkylene-NR^{a}—, —C(O)—C₁-C₁₂ alkylene, —C₁-C₁₂ alkylene-C(O)—, —C₁-C₁₂ alkylene-NR^{a}—C(O)—, —C₁-C₁₂ alkylene-C(O)—NR^{a}—C₁-C₁₂ alkylene-, —NR^{a}—C₁-C₁₂ alkylene-C(O)—, —C(O)—C₁-C₁₂ alkylene-NR^{a}—, —NR^{a}—C(O)—NR^{a}—, —NR^{a}—C(O)—, —NR^{a}—C(O)—C₁-C₁₂ alkylene, —C(O)—NR^{a}—C₁-C₁₂ alkylene, -heteroarylene, heteroaryl-C₁-C₁₂ alkylene, heteroaryl-C₁-C₁₂ alkylene-C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH₂, —O—C(O)—NR^{a}—C₁-C₁₂ alkylene, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁ 8-, triazolyl, —C₁-C₁₂ alkylene-triazolyl-, and —C(O)NR⁷R⁸, wherein one of R⁷ and R⁸ is H or C₁-C₁₂ alkylene and the other is C₁-C₁₂ alkylene, each R^{a} is independently selected from H, C₁₋₆ alkyl, and wherein any of the above alkylene groups may be substituted with —SO₃H;
   each R⁴ and R⁵ are independently H, a polyhydroxyl group, a carboxyl-containing moiety, a substituted polyhydroxyl group, a —C(O)-polyhydroxyl group, a substituted —C(O)— polyhydroxyl group, a polyhydroxyl-ether group, a substituted polyhydroxyl-ether group, or a chelator, wherein optional substituents are selected from sulfate, phosphate, alkyl sulfate, and alkyl phosphate, and wherein at least one of R⁴ and R⁵ is not H;
   each R⁶ is independently a bond or selected from:

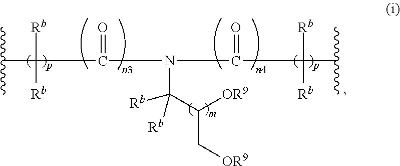

wherein:
   each n³ and n⁴ are independently 0-1,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6,
   m is 1-4, and
each v is independently 1-6, and
   n² is 1;

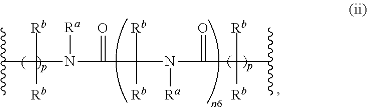

wherein:
   each R^{a} is independently H or C₁₋₆ alkyl,
   each R^{b} is independently H or C₁₋₆ alkyl,
   n⁶ is 1-10, and
   each p is independently 0-6, and
n² is 1;

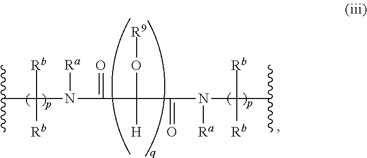

wherein:
   each R^{a} is independently H or C₁₋₆ alkyl,
   each R^{b} is independently H or C₁₋₆ alkyl,
   each R⁹ is independently H, acetyl, —P(═O)(OH)₂, or —(CH₂)ᵥ—O—S(═O)₂(OH),
   each p is independently 0-6, and
   q is 1-8,
each v is independently 1-6, and
   n² is 1;

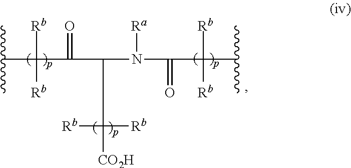

wherein:
   each R^{a} is independently H or C₁₋₆ alkyl,
   each R^{b} is independently H or C₁₋₆ alkyl, and
   each p is independently 0-6, and
n² is 1;
(v) —R¹⁰—[O—CH₂—CH₂]₁₋₈—R¹⁰—, wherein:
   each R^{b} is independently H or C₁₋₆ alkyl,
      each R¹⁰ is independently

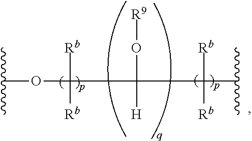

each p is independently 1-6, and
q is 1-8; and
(vi) —N—(R¹—X—R²—[O—CH₂—CH₂]ₙ₀—R²—R³—(NR⁴R⁵)ₙ₁)₂, wherein:
   each X is independently —NR^{a}—C(O)— or —C(O)NR^{a}—, and
   n² is 2; and
   the wavy line (˜) indicates the attachment site of the amino acid unit to R⁰;
   each n⁰ is independently 2-26;
   n¹ is 0-6, and when n¹ is 0 then R³ is —OH or —C(O)OR^{b}, wherein R^{b} is independently H or C₁₋₆ alkyl; and
   n³ is 1-6.

Embodiment 7. The linker compound of any one of Embodiments 1 and 4-6, wherein each R³ is independently selected from a bond, —C(O)—, —NR^{a}—C(O)—C₁-C₁₂ alkylene-C(O)—, —C(O)—NR^{a}—C₁-C₁₂ alkylene-(CH(OH))₁₋₈—C₁-C₁₂ alkylene-, —O—CH₂—CH(OH)—C(O)—, —O—CH₂—CH(OH)—C(O)—NR^{a}—C₁-C₁₂ alkylene-, —CH(OH)—, —CH(OH)—C₁-C₁₂ alkylene-, C₁-C₁₂ alkylene-CH(OH)—, —CH(OH)—C(O)—, —CH(OH)—C(O)—NR^{a}—C₁-C₁₂ alkylene-, —CH(OH)—C₁-C₁₂ alkylene-NR^{a}—C(O)—C₁-C₁₂ alkylene-C(O)—NR^{a}—C₁-C₁₂ alkylene-, —NR^{a}—C(O)—C₁-C₁₂ alkylene-C(O)—NR^{a}—C₁-C₁₂ alkylene-, —CH(OH)—NR^{a}—C₁-C₁₂ alkylene-, —[C(O)—(CH₂)₁₋₈—NR^{a}]₁₈—, triazolyl, and —C₁-C₁₂ alkylene-triazolyl-, —N(polyhydroxyl group)-, each R^{a} is independently selected from H, C₁₋₆ alkyl; and wherein any of the above alkylene groups may be substituted with —SO₃H.

Embodiment 8. The linker compound of any one of Embodiments 1-3 and 5-7, wherein the linker unit comprises a moiety selected from:

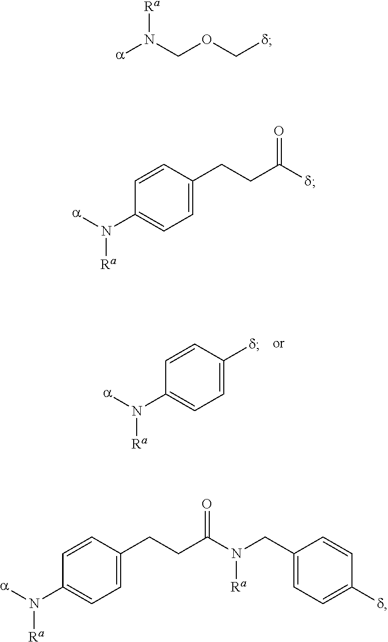

or a stereoisomer or salt thereof, wherein:
   α—represents a direct or indirect attachment site to the amino acid unit or;
   δ—represents an attachment site to at least one of the drug units or an attachment site to a linking group attached to the at least one of the drug units; and
      R^{a} is H or C₁₋₆ alkyl.

Embodiment 9. A linker compound, or a stereoisomer or salt thereof, comprising:
(a) a linker unit having from 1 to 4 attachment sites for a drug unit and having one of the following structures (i) or (ii):

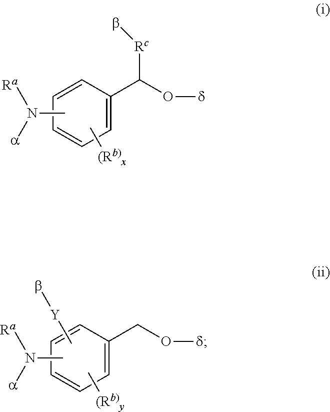

(b) at least one polar group comprising a polymer unit, optionally a sugar unit, optionally a carboxyl unit, and combinations thereof; and
(c) optionally a stretcher group having an attachment site for a SLITRK6 binding agent;
   wherein:
α—is an attachment site to an enzyme-cleavable group;
β—is an attachment site to the at least one polar group;
δ—is H, an attachment site to at least one of the drug units, or an attachment site to a linking group attached to the at least one of the drug units;
the polymer unit comprises a polyamide, a polyether, or a combination thereof, wherein the polyether comprises a hydroxyl group, a polyhydroxyl group, a sugar group, a carboxyl group, or combinations thereof;
each R^{a} independently is H or C₁-C₆ alkyl;
each R^{b} independently is halo, C₁₋₆ alkyl, an attachment site to at least one of the drug units, or an attachment site to at least one of the polar groups;
x is 0, 1, 2, 3 or 4;
y is 0, 1, 2 or 3;
R^{c} is a bond, —C(O)—, —S(O)—, —SO₂—, C₁₋₆ alkylene, C₁₋₆ alkynylene, triazolyl or combinations thereof; and
Y is a bond, —O—, —S—, —N(R^{a})—, —C(O)—, —S(O)—, —SO₂—C₁-C₆ alkylene, C₁-C₆ alkenylene, C₁-C₆ alkynylene, triazolyl, a group containing triazolyl, or combinations thereof.

Embodiment 10. The linker compound of Embodiment 9, wherein the linker unit has one of the following structures (i-a), (ii-a), or (iii-a):

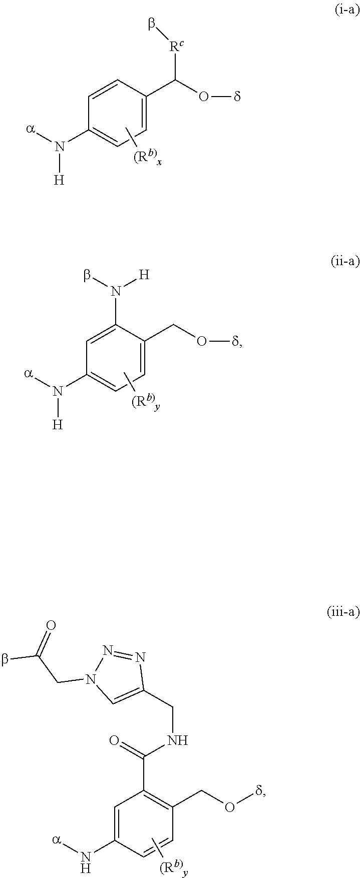

or a stereoisomer or salt thereof.

Embodiment 11. The linker compound of Embodiment 9 or 10, wherein the linker unit has one of the following structures (i-b), (i-c), (i-d), (i-e) or (i-f):

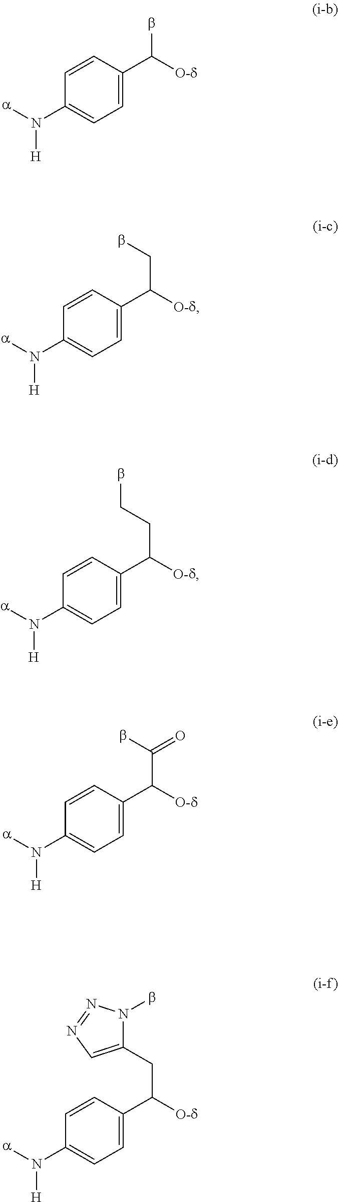

or a stereoisomer or salt thereof.

Embodiment 12. The linker compound of Embodiments 9, wherein the linker unit has the following structure (ii-b) or (iii-b):

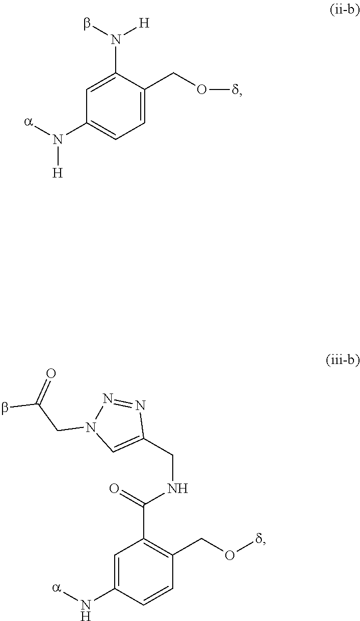

or a stereoisomer or salt thereof.

Embodiment 13. The linker compound of any one of Embodiments 9-12, wherein the polar group comprises at least one sugar unit having the following formula:

L3-N(CH₂—(CH(XR))ₖ—X₁(X₂))₂ (X)
or a stereoisomer or salt thereof, wherein:
   each X is independently selected from NH and O;
   each R is independently selected from hydrogen, acetyl, a monosaccharide, a
   disaccharide, and a polysaccharide;
   each X₁ is independently selected from CH₂ and C(O);
   each X₂ is independently selected from H, OH and OR;
   k is 1 to 10; and
   L3 is a point of attachment to a remainder of the polar group.

Embodiment 14. The linker compound of Embodiment 13, wherein the at least one sugar unit has one of the following structures (XII) or (XIII):

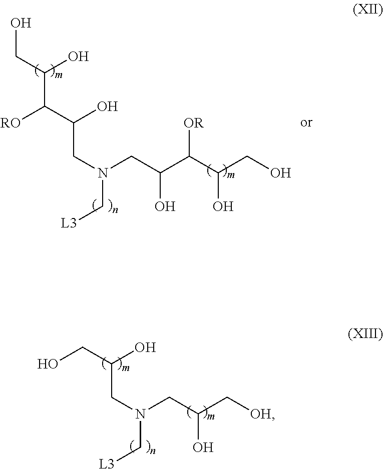

or a stereoisomer or salt thereof, wherein:
   each R is independently selected from hydrogen, a monosaccharide, a disaccharide and a polysaccharide;
   m is 1 to 8; and
   n is 0 to 4.

Embodiment 15. The linker compound of any one of Embodiments 1-14, the polar group has a formula selected from:

(a) ˜R²⁰—R²¹—[O—CH₂—CH₂]ₙ₂₀—R²²—NR²⁴R²⁵ (XX)
or a stereoisomer a salt thereof, wherein:
R²⁰ is an attachment group to site β or to site R^{b}, or to the enzyme-cleavable group;
R²¹ and R²² are each, independently, a bond or C₁-C₃ alkylene;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); or —NR²⁴R²⁵ together from a C₃-C₈ heterocycle; and
n20 is 2 to 26; or

(b) ˜R²⁰—R²¹—[O—CH₂—CH₂]ₙ₂₀—R²²—NR²⁴R²⁵ (XXI)
or a stereoisomer or salt thereof, wherein:
R²⁰ is an attachment group to site p or to site R^{b}, or to the enzyme-cleavable group;
R²¹ and R²² are each, independently, a bond or C₁-C₃ alkylene;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); and the other of R²⁴ and R²⁵ is a polyethylene glycol, optionally having 1 to 24 ethylene glycol subunits; and
n20 is 2 to 26; or

(c) ˜R²⁰-[—R²⁶—[R²⁹—[O—CH₂—CH₂-]ₙ₂OR²⁹]ₙ₂₁—R²⁷—NR²⁴R²⁵]ₙ₂₇ (XXII)
or a stereoisomer or salt thereof, wherein:
R²⁰ is an attachment group to site p or to site R^{b}, or to the enzyme-cleavable group;
R²⁶ and R²⁷ are each optional and are, independently, selected from a bond, C₁-C₁₂ alkylene, —NH—C₁-C₁₂ alkylene, —C₁-C₁₂ alkylene-NH—, —C₁-C₁₂ alkylene-N(CH₃)—, —C(O)—C₁-C₁₂ alkylene, —C₁-C₁₂ alkylene-C(O)—, —NH—C₁-C₁₂ alkylene-C(O)— and —C(O)—C₁-C₁₂ alkylene-NH—;
one of R²⁴ and R²⁵ is selected from a H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is the sugar unit of formula (XII) or (XIII); and the other of R²⁴ and R²⁵ is selected from H; polyhydroxyl group; substituted polyhydroxyl group; —C(O)-polyhydroxyl group; substituted —C(O)-polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); and polyethylene glycol, optionally having 1 to 24 ethylene glycol subunits; or —NR²⁴R²⁵ together from a C₃-C₈ heterocycle;
each R²⁹ is optional and independently selected from —C(O)—, —NH—, —C(O)—C₁-C₆ alkylene-, —NH—C₁-C₆ alkylene-, —C₁-C₆ alkylene-NH—, —C₁-C₆ alkylene-C(O)—, —NH(CO)—C₁-C₆alkylene-, —N(CH₃)—(CO)—C₁-C₆alkylene-, —NH(CO)NH—, and triazole;
n20 is 2 to 26;
n21 is 1 to 4; and
n27 is 1 to 4, or

(d) ˜-R²⁰—R²¹—[—C(R^{α})H—C(O)—N(R^{N})—]ₙ₂₀—R²²—NR²⁴R²⁵ (XXIII)
or a stereoisomer or salt thereof, wherein:
R²⁰ is an attachment group to site p or to site R^{b}, or to the enzyme-cleavable group;
R²¹ is a bond, C₁-C₃ alkylene,
   —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂—]ₙ₂₀—C(O)—;
R²² is C₁-C₃ alkylene,
   —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); or —NR²⁴R²⁵ together from a C₃-C₈ heterocycle; and
each n20 is independently 2 to 26, or

(e) ˜R²⁰—R²¹—[—C(R^{α})H—C(O)—N(R^{N})—]ₙ₂₀—R²²—CO₂R²⁶ (XXIV)
or a stereoisomer or salt thereof, wherein:
R²⁰ is an attachment group to site p or to site R^{b}, or to the enzyme-cleavable group;
R²¹ and R²² are each, independently, a bond, C₁-C₃ alkylene, or —C₁-C₃alkylene[O—CH₂—CH₂-]n2o;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); or —NR²⁴R²⁵ together from a C₃-C₈ heterocycle;
R²⁶ is H or C₁-C₄ alkyl; and
each n20 is independently 2 to 26,
with the proviso that at least one R^{α} or R^{N} is —R²²—NR²⁴R²⁵; or

(f) ˜R²⁰—R²¹—[C(R^{α})H—C(O)—N(R^{N})—]ₙ₂₀-R²²—N—(R²³—NR²⁴R²⁵)₂ (XXV)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, or —C₁-C₃alkylene-[O—CH₂—CH₂-]ₙ₂₀;
each R^{α} is independently H or —R²²—NR²⁴R²⁵;
each R^{N} is independently H or C₁-C₆ alkyl;
each R²³ is independently C₁-C₆ alkylene;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; and —C(O)—R²⁸, where R²⁸ is the sugar unit of formula (XII) or (XIII); or —NR²⁴R²⁵ together from a C₃-C₈ heterocycle; and
each n20 is independently 2 to 26.

Embodiment 16. The linker compound of Embodiment 15, wherein R²⁴ and R²⁵ are each independently selected from H and a polyhydroxyl group, provided that R²⁴ and R²⁵ are not both H.

Embodiment 17. The linker compound of Embodiment 15 or 16, wherein the polyhydroxyl group is a linear monosaccharide, optionally selected from a C₆ or C₅ sugar, a sugar acid and an amino sugar.

Embodiment 18. The linker compound of Embodiment 17, wherein:
the C6 or C5 sugar is selected from glucose, ribose, galactose, mannose, arabinose, 2-deoxyglucose, glyceraldehyde, erythrose, threose, xylose, lyxose, allose, altrose, gulose, idose, talose, aldose, and ketose;
the sugar acid is selected from gluconic acid, aldonic acid, uronic acid and ulosonic acid; or
the amino sugar is selected from glucosamine, N-acetyl glucosamine, galactosamine, and N-acetyl galactosamine.

Embodiment 19. The linker compound of any one of Embodiments 15 to 18, the polar group is selected from the following, or a stereoisomer or salt thereof:

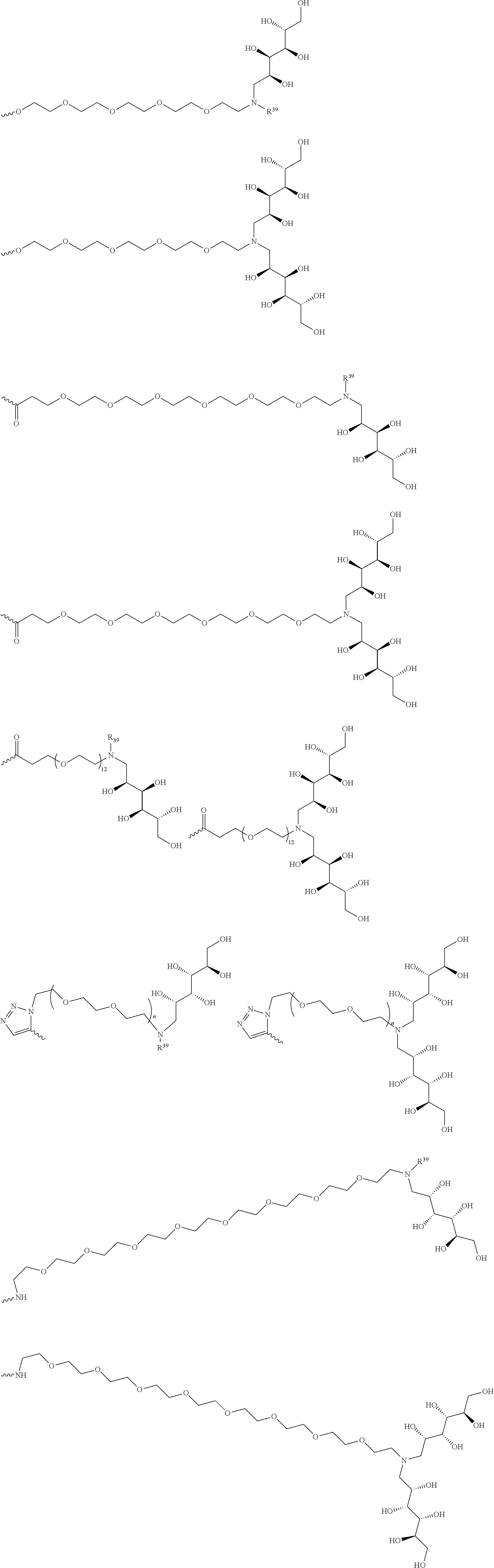

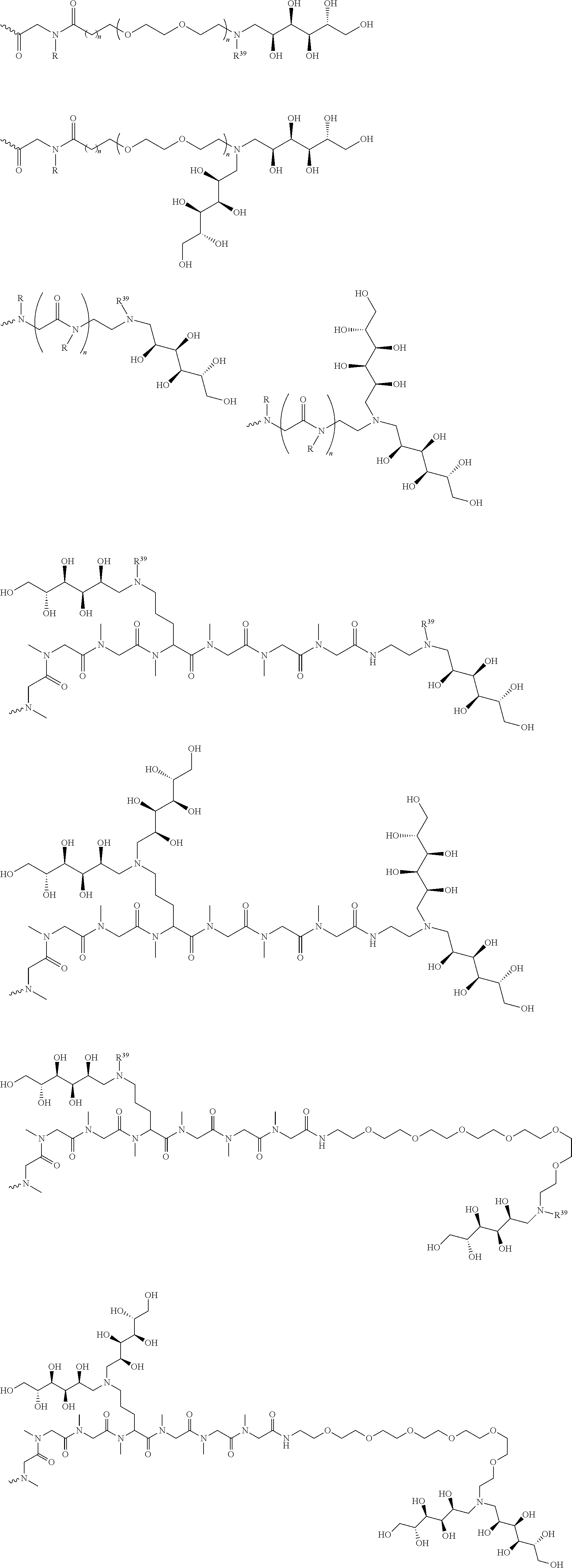

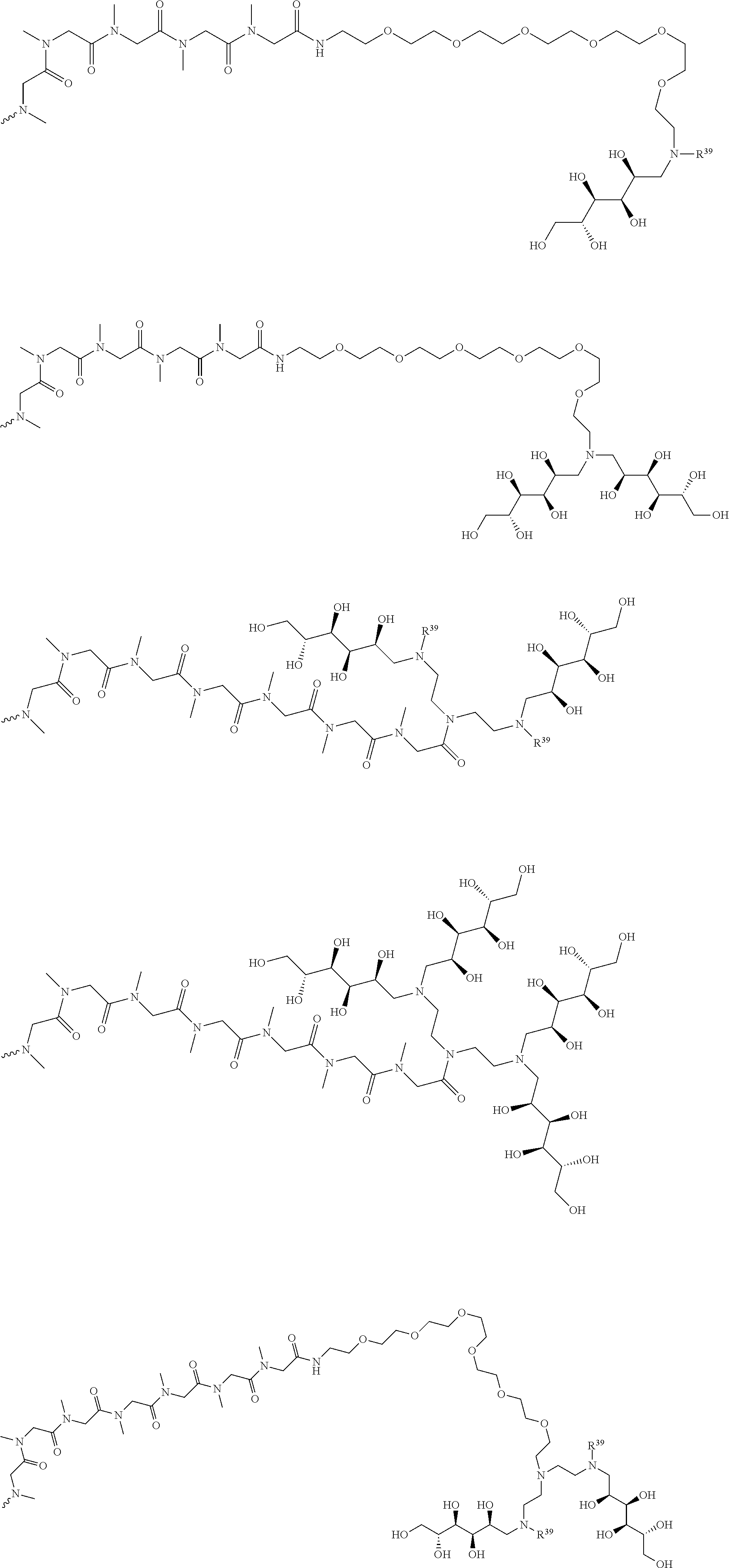

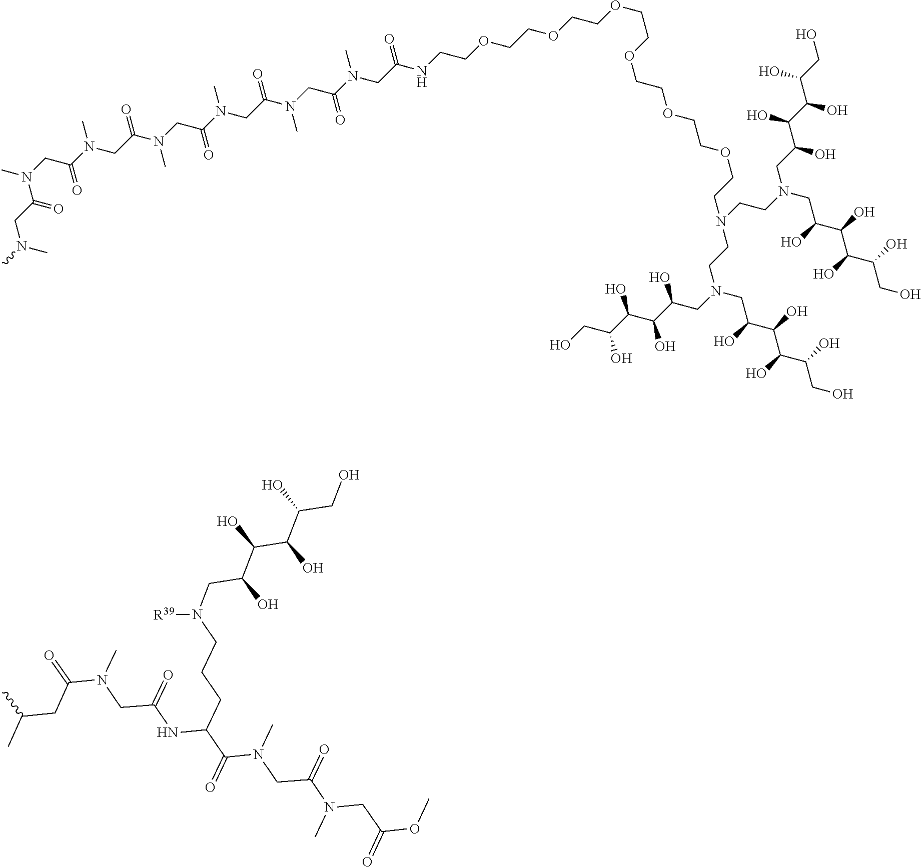

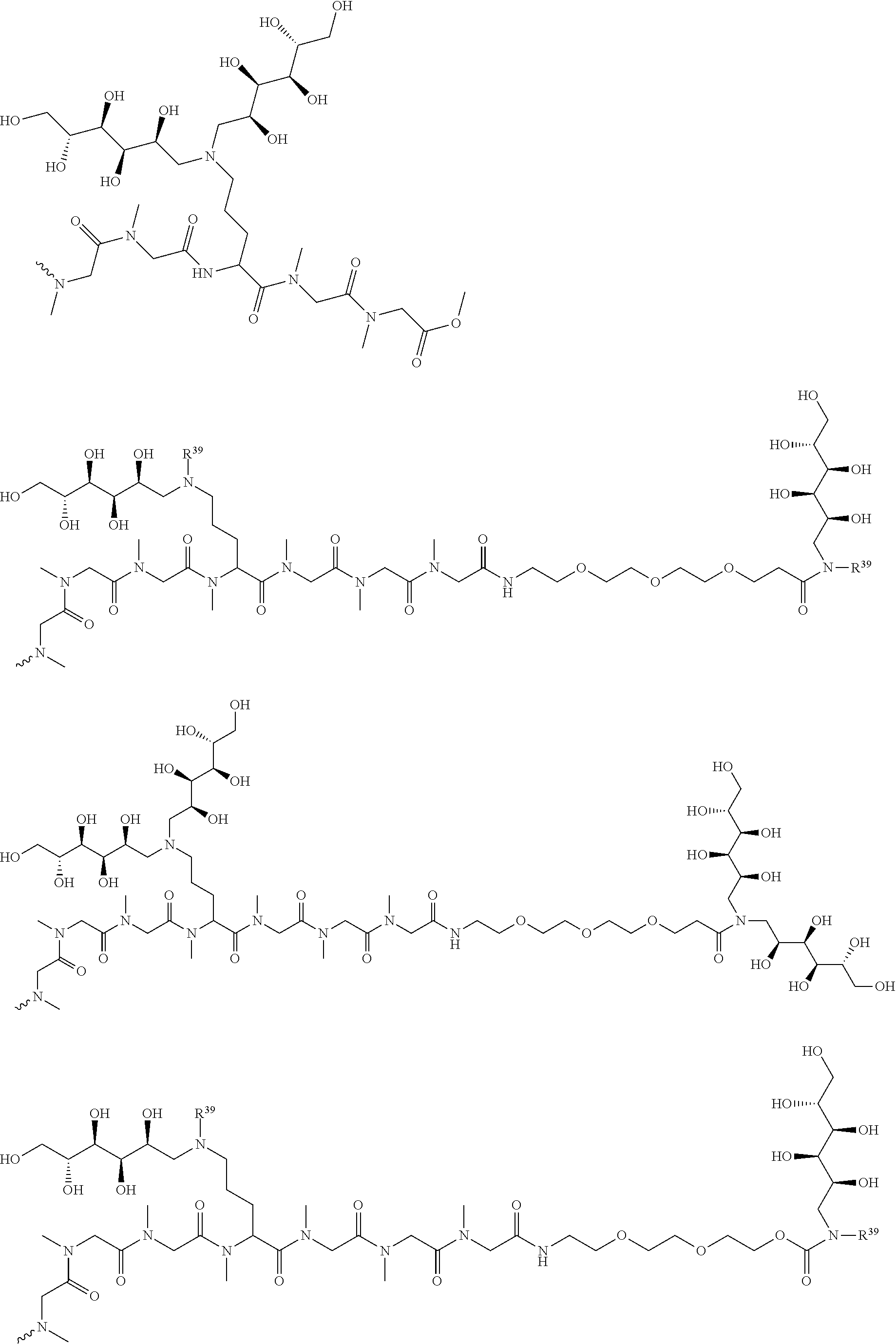

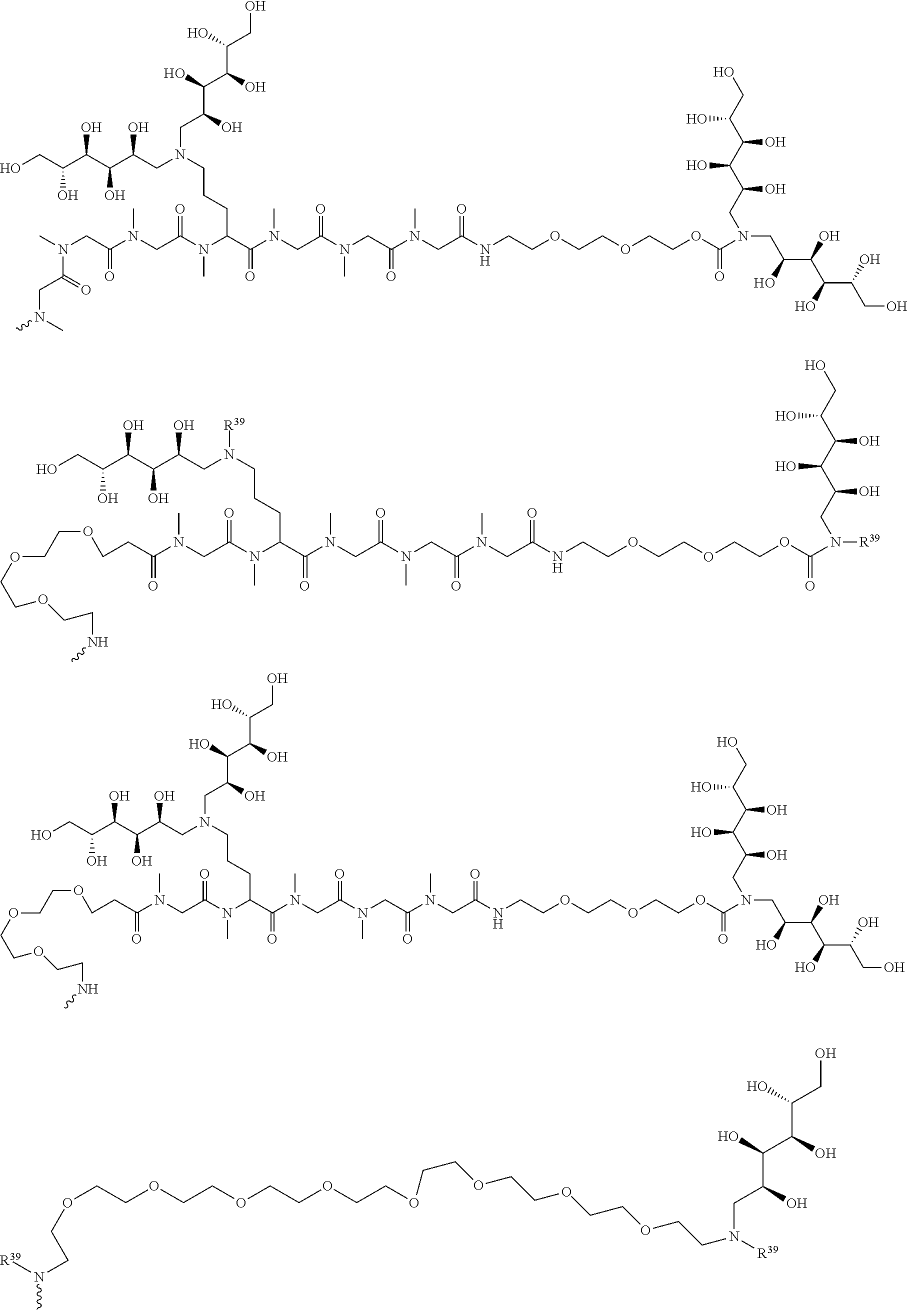

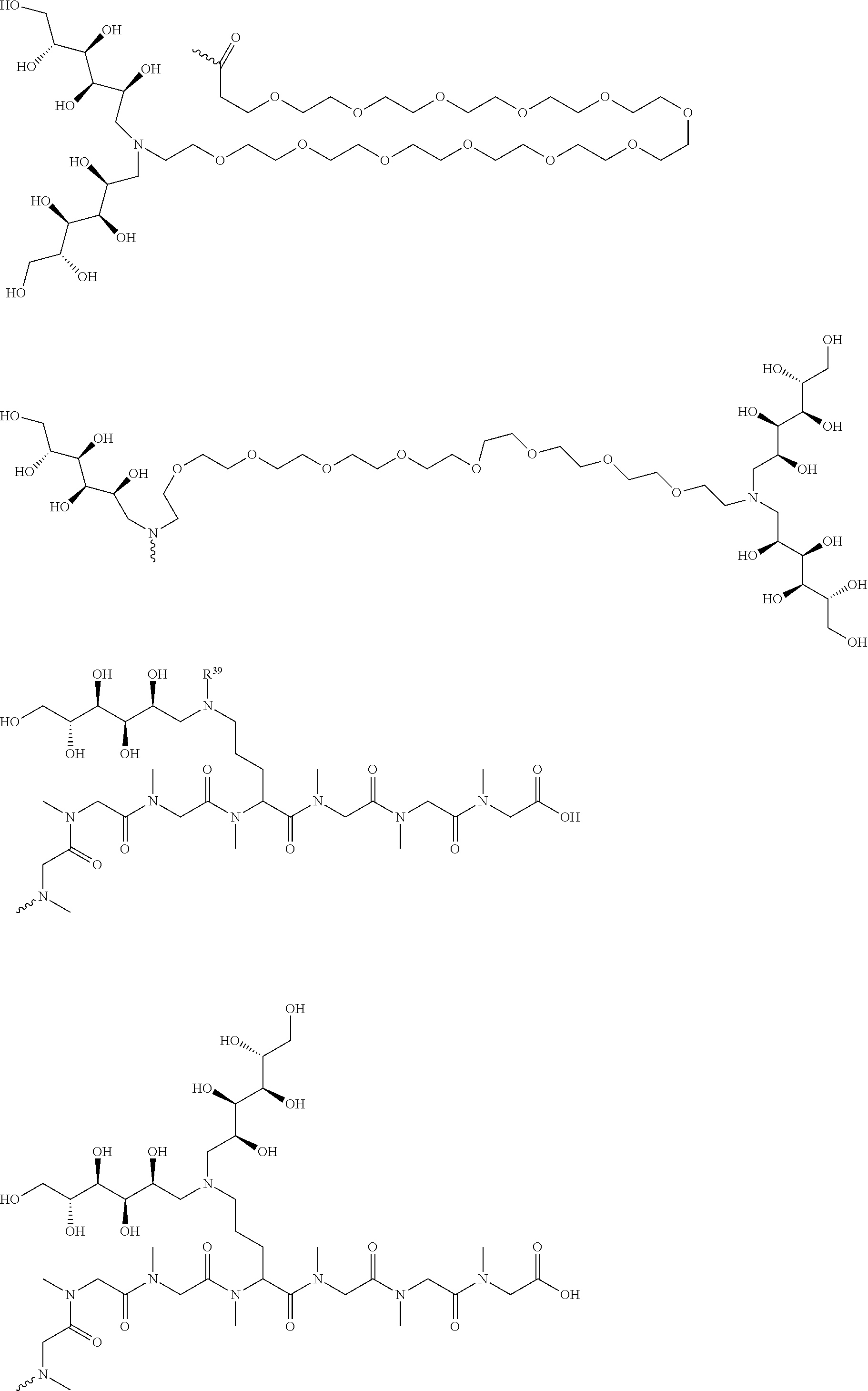

wherein each R is independently H or alkyl; each R³⁹ is independently selected from H, a linear monosaccharide and polyethylene glycol, optionally having from 1 to 24 ethylene glycol subunits; each n independently is 1-12; and the wavy line is an attachment to site β or to site R^{b}, or to the enzyme-cleavable group.

Embodiment 20. The linker compound of Embodiment 15 or 16, wherein one of R²⁴ and R²⁵ is a linear monosaccharide and the other is a cyclic monosaccharide.

Embodiment 21. The linker compound of Embodiment 20, wherein-(NR²⁴R²⁵) is selected from the following, or a stereoisomer or salt thereof:

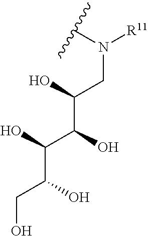

wherein R¹¹ is a cyclic monosaccharide.

Embodiment 22. The linker compound of Embodiment 20, the polar group is selected from the following, or a stereoisomer or salt thereof:

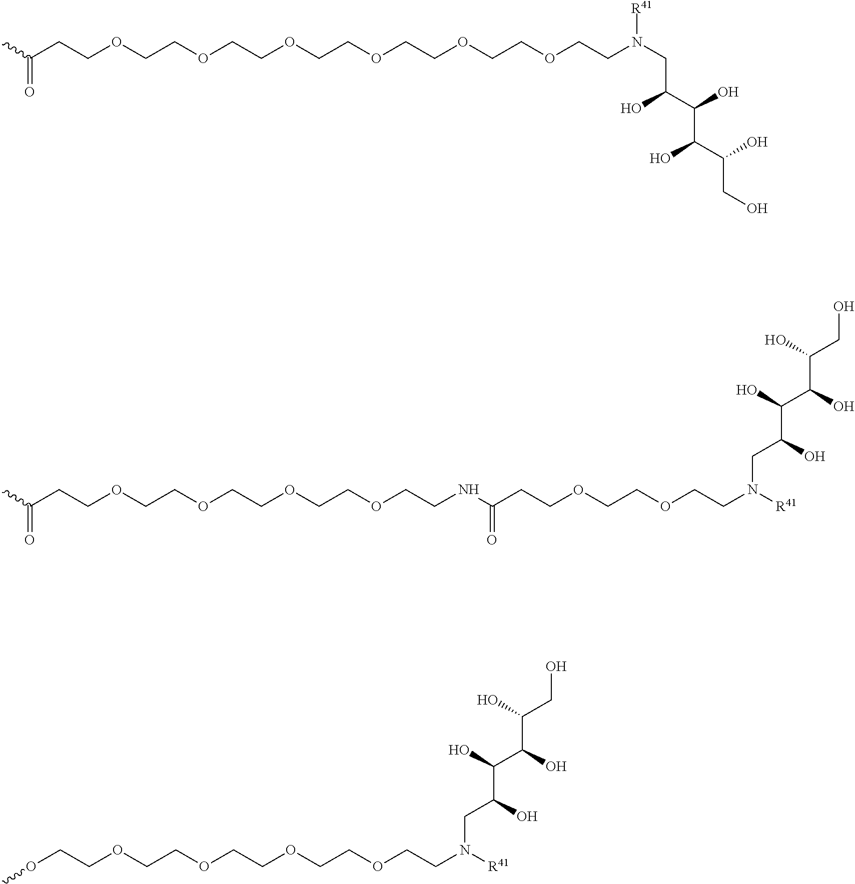

wherein R⁴¹ is a cyclic monosaccharide; and the wavy line is an attachment site to R^{b}, or to the enzyme-cleavable group.

Embodiment 23. The linker compound of Embodiment 15, wherein R²⁴ and R²⁵ are independently a polyhydroxyl selected from a cyclic monosaccharide, disaccharide, and polysaccharide.

Embodiment 24. The linker compound of Embodiment 23, wherein —(NR²⁴R²⁵) is selected from the following, or a stereoisomer or salt thereof:

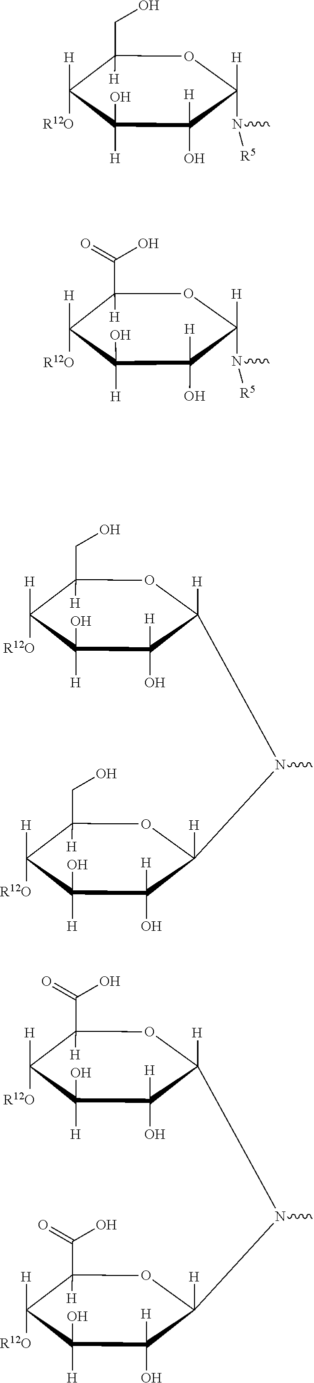

wherein each R¹² is selected from H and a monosaccharide, a disaccharide, or a polysaccharide; and R⁵ is selected from a cyclic monosaccharide, disaccharide, or polysaccharide.

Embodiment 25. The linker compound of Embodiment 23, the polar group is selected from the following, or a stereoisomer or salt thereof:

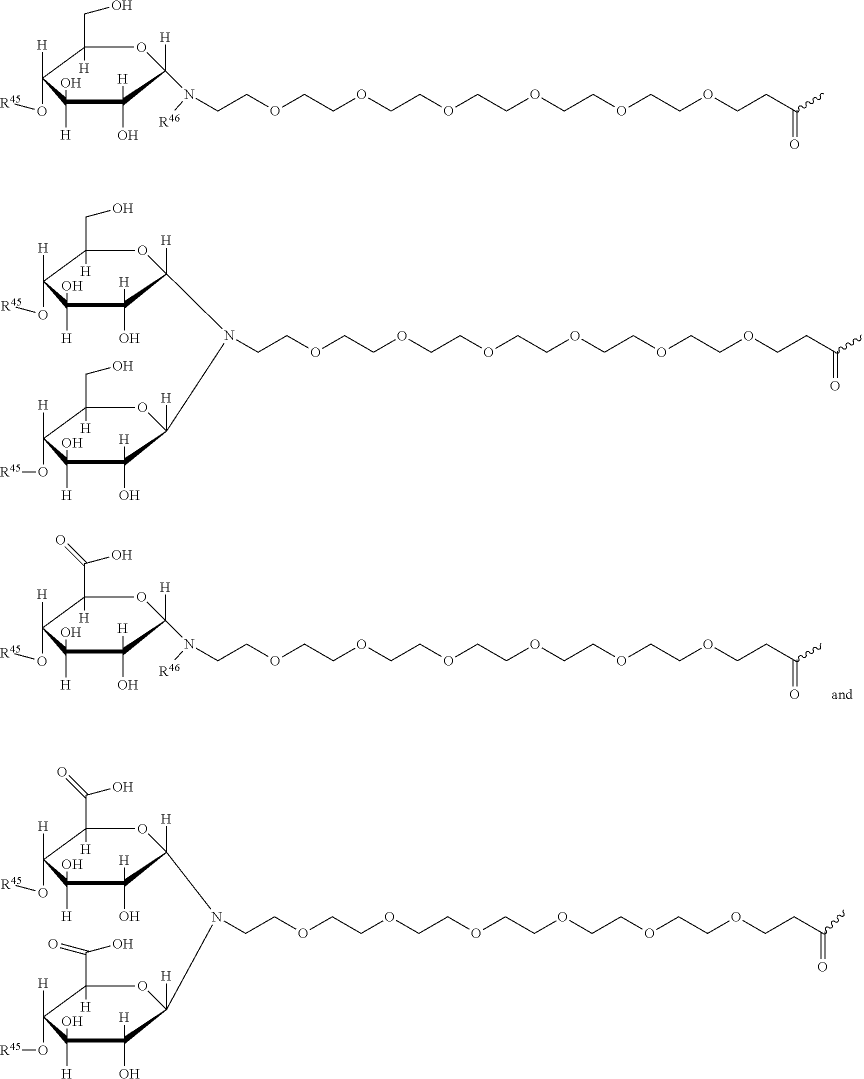

wherein each R⁴⁵ is selected from H and a monosaccharide, a disaccharide, or a polysaccharide; and R⁴⁶ is selected from a cyclic monosaccharide, disaccharide, or polysaccharide; and the wavy line is an attachment site to R^{b}, or to the enzyme-cleavable group.

Embodiment 26. The linker compound of Embodiment 15, wherein R²⁴ and R²⁵ are independently selected from a linear monosaccharide and a substituted linear monosaccharide, wherein the substituted linear monosaccharide is substituted with a monosaccharide, a disaccharide, or a polysaccharide.

Embodiment 27. The linker compound of Embodiment 26, wherein —(NR²⁴R²⁵) is selected from the following, or a stereoisomer or salt thereof:

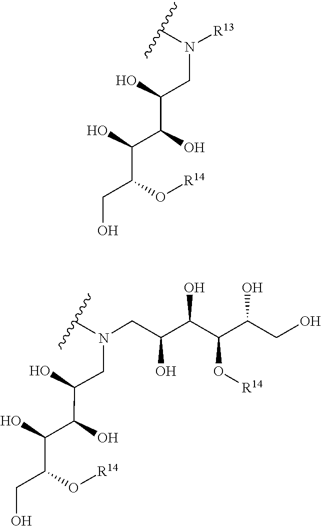

wherein R¹³ is a linear monosaccharide; and each R¹⁴ is selected from a monosaccharide, a disaccharide and a polysaccharide.

Embodiment 28. The linker compound of Embodiment 26, the polar group is selected from the following, or a stereoisomer or salt thereof:

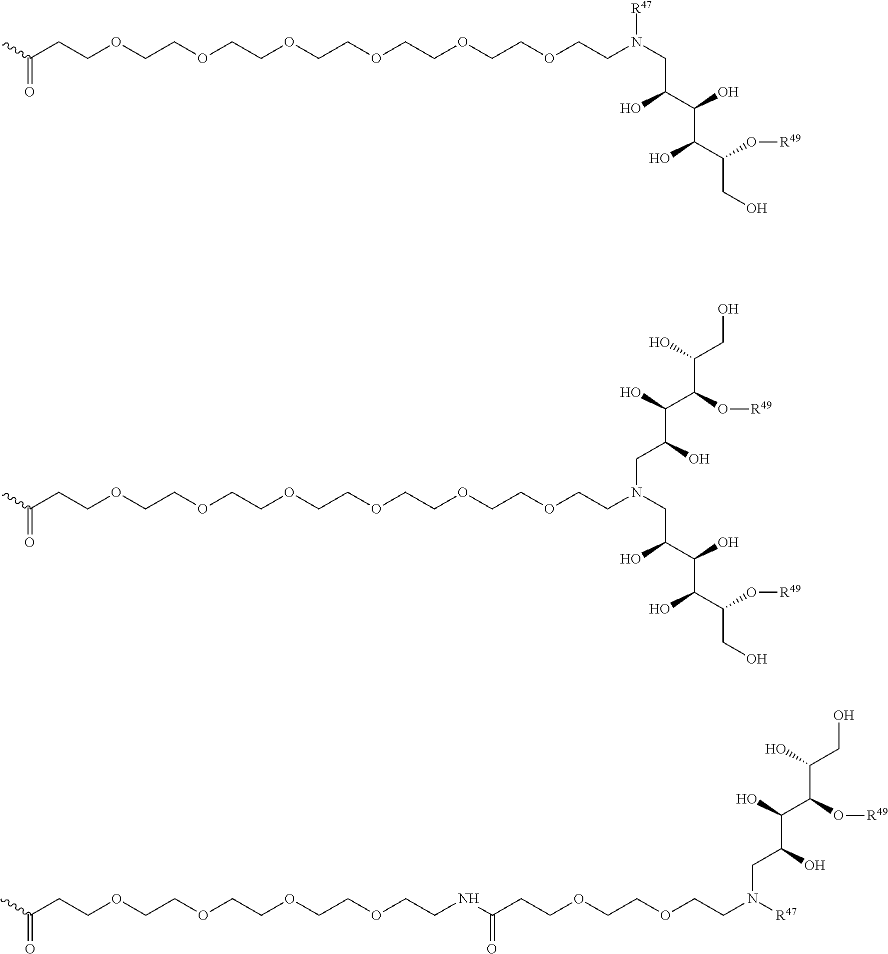

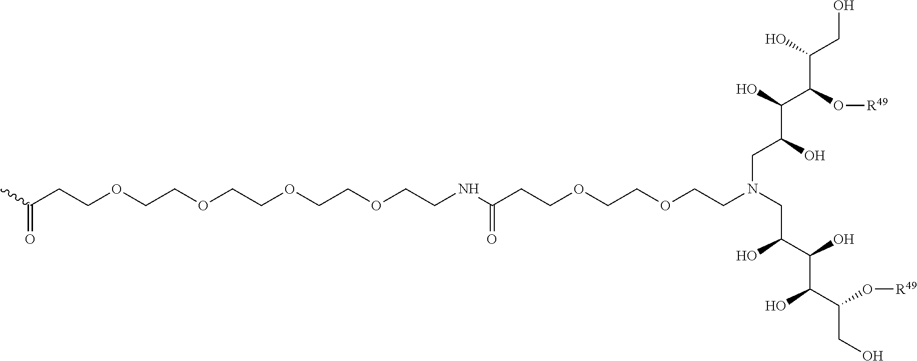

wherein R⁴⁷ is a linear monosaccharide; and each R⁴⁹ is selected from a monosaccharide, a disaccharide, and a polysaccharide; and the wavy line is an attachment site to R^{b}, or to the enzyme-cleavable group.

Embodiment 29. The linker compound of Embodiment 15, wherein R²⁴ and R²⁵ are independently selected from a linear monosaccharide and a substituted monosaccharide, wherein the substituted linear monosaccharide is substituted with one or more substituents selected from carboxyl, ester, and amide, and optionally further substituted with a monosaccharide, disaccharide, or a polysaccharide.

Embodiment 30. The linker compound of Embodiment 29, wherein —(NR²⁴R²⁵) is selected from the following, or a stereoisomer or salt thereof:

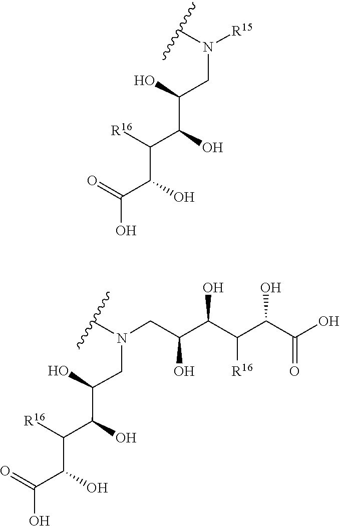

wherein each R¹⁵ is independently selected from a linear monosaccharide and a substituted linear monosaccharide; each R¹⁶ is independently selected from hydroxyl, carboxyl, ester, and amide.

Embodiment 31. The linker compound of Embodiment 29, the polar group is selected from the following, or a stereoisomer or salt thereof:

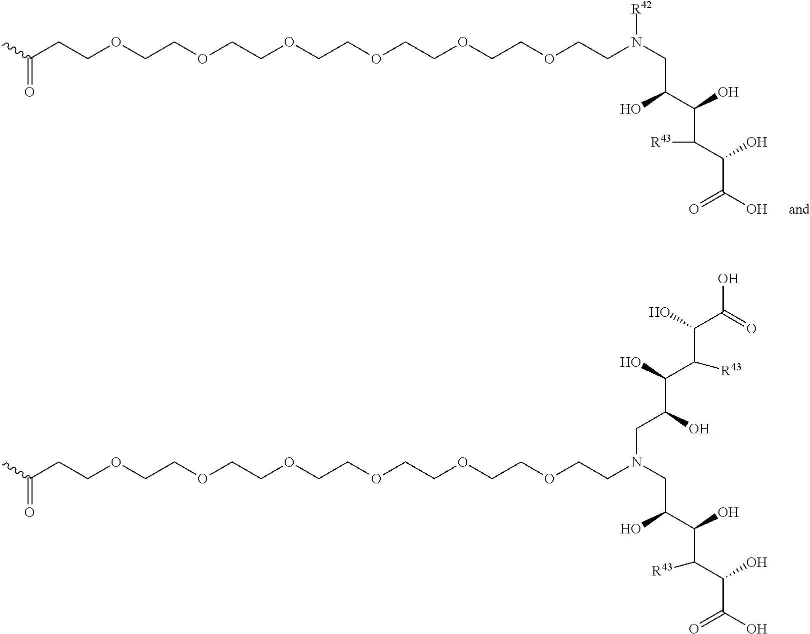

wherein each R⁴² is independently selected from a linear monosaccharide and a substituted linear monosaccharide; each R⁴³ is independently selected from hydroxyl, carboxyl, ester, and amide; and the wavy line is an attachment site to R^{b}, or to the enzyme-cleavable group.

Embodiment 32. The linker compound of Embodiment 15, wherein one of R²⁴ and R²⁵ is a —C(O)-polyhydroxyl group or substituted —C(O)-polyhydroxyl group, and the other of R²⁴ and R²⁵ is a H, —C(O)-polyhydroxyl group, substituted —C(O)-polyhydroxyl group, polyhydroxyl group or substituted polyhydroxyl group; wherein the substituted —C(O)-polyhydroxyl group and polyhydroxyl group are substituted with a monosaccharide, a disaccharide, a polysaccharide, carboxyl, ester, or amide.

Embodiment 33. The linker compound of Embodiment 32, wherein —(NR²⁴R²⁵) is selected from the following, or a stereoisomer or salt thereof:

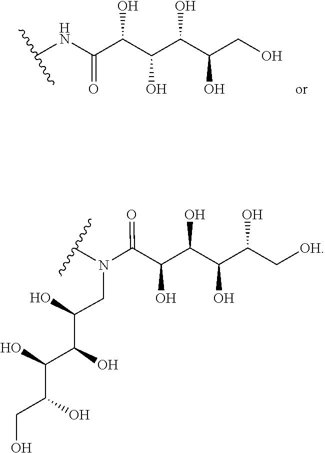 [SMILES: *NC(=O)[C@H](O)[C@@H](O)[C@H](O)[C@H](O)CO ; "or" ; *N(C[C@H](O)[C@@H](O)[C@H](O)[C@H](O)CO)C(=O)[C@H](O)[C@@H](O)[C@H](O)[C@H](O)CO]

Embodiment 34. The linker compound of Embodiment 32, the polar group is selected from the following, or a stereoisomer or salt thereof:

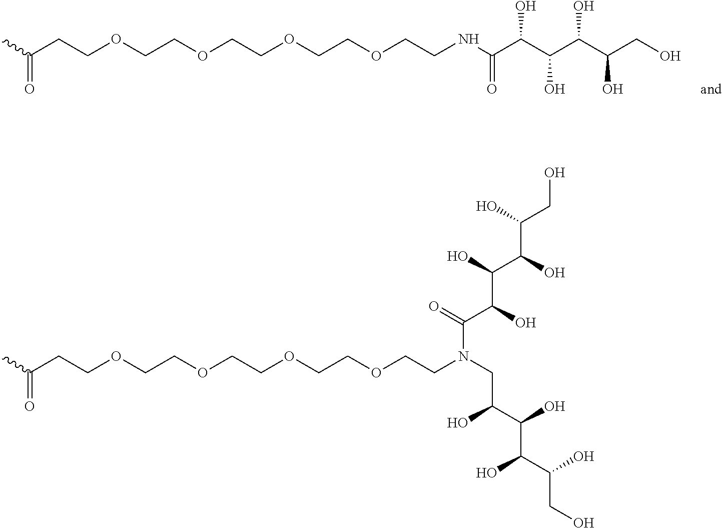

wherein the wavy line is an attachment site to R^{b}, or to the enzyme-cleavable group.

Embodiment 35. The linker compound of Embodiment 15, wherein wherein —(NR²⁴R²⁵) is selected from the following, or a stereoisomer or salt thereof:

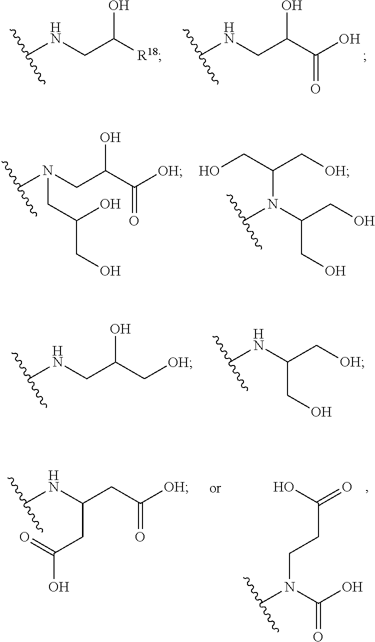

wherein R¹⁸ is selected from OH, CH₂OH, COOH or —C₁-C₆ alkyl substituted with hydroxyl or carboxyl.

Embodiment 36. The linker compound of Embodiment 15, wherein R²⁴ and R²⁵ are independently H or substituted —C₁-C₈ alkyl, provided that both R²⁴ and R²⁵ are not H; wherein substituted —C₁-C₈ alkyl is substituted with hydroxyl and/or carboxyl.

Embodiment 37. The linker compound of Embodiment 36, the polar group is selected from the following, or a stereoisomer or salt thereof:

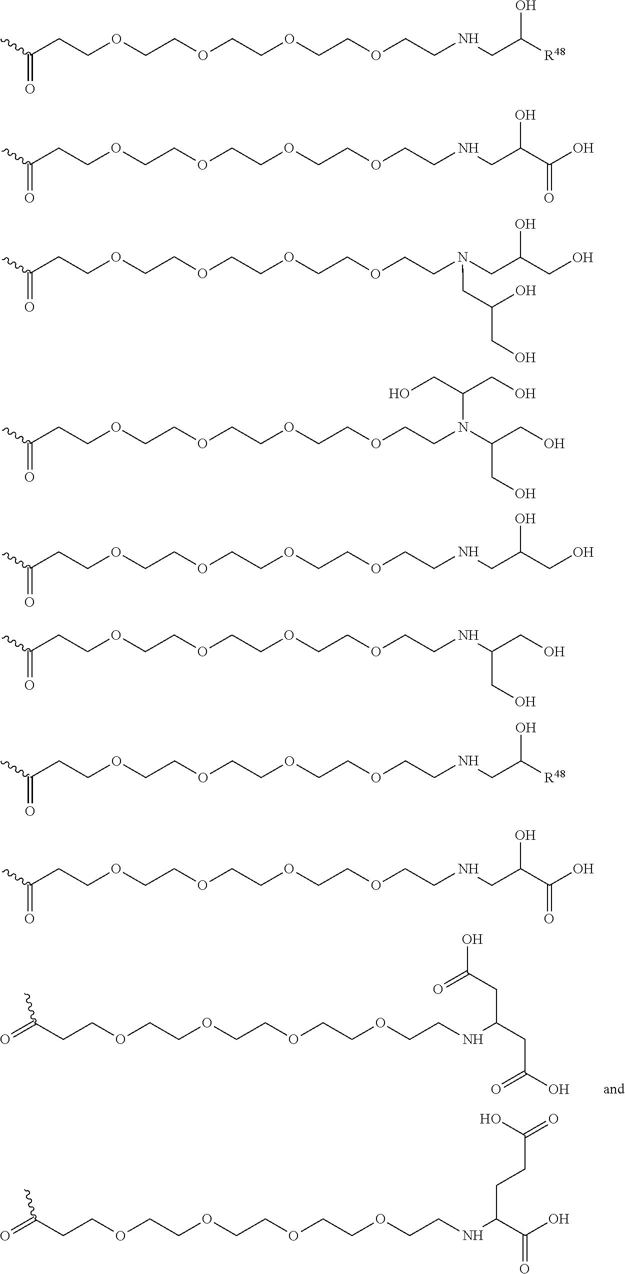

wherein R⁴⁸ is selected from H, OH, CH₂OH, COOH, or —C₁-C₆ alkyl substituted with hydroxyl or carboxyl; and the wavy line is an attachment site to R^{b}, or to the enzyme-cleavable group.

Embodiment 38. The linker compound of Embodiment 15, wherein —(NR²⁴R²⁵) is selected from the following, or a stereoisomer or salt thereof:

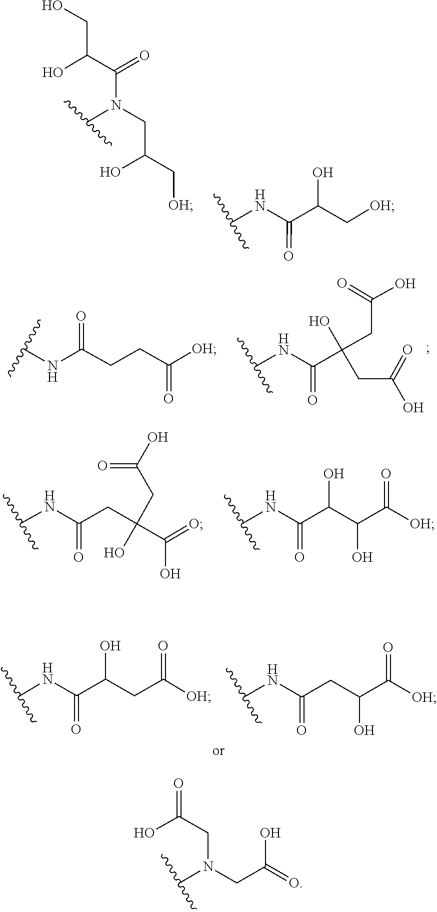

Embodiment 39. The linker compound of Embodiment 15, wherein one of R²⁴ and R²⁵ is H or substituted —C(O)—C₁-C₈ alkyl, and the other of R²⁴ and R²⁵ is substituted —C(O)—C₁-C₈ alkyl, or substituted —C₁-C₈ alkyl, wherein substituted —C(O)—C₁-C₈ alkyl and substituted —C₁-C₈ alkyl, are substituted with hydroxyl and/or carboxyl.

Embodiment 40. The linker compound of Embodiment 39, the polar group is selected from the following, or a stereoisomer or salt thereof:

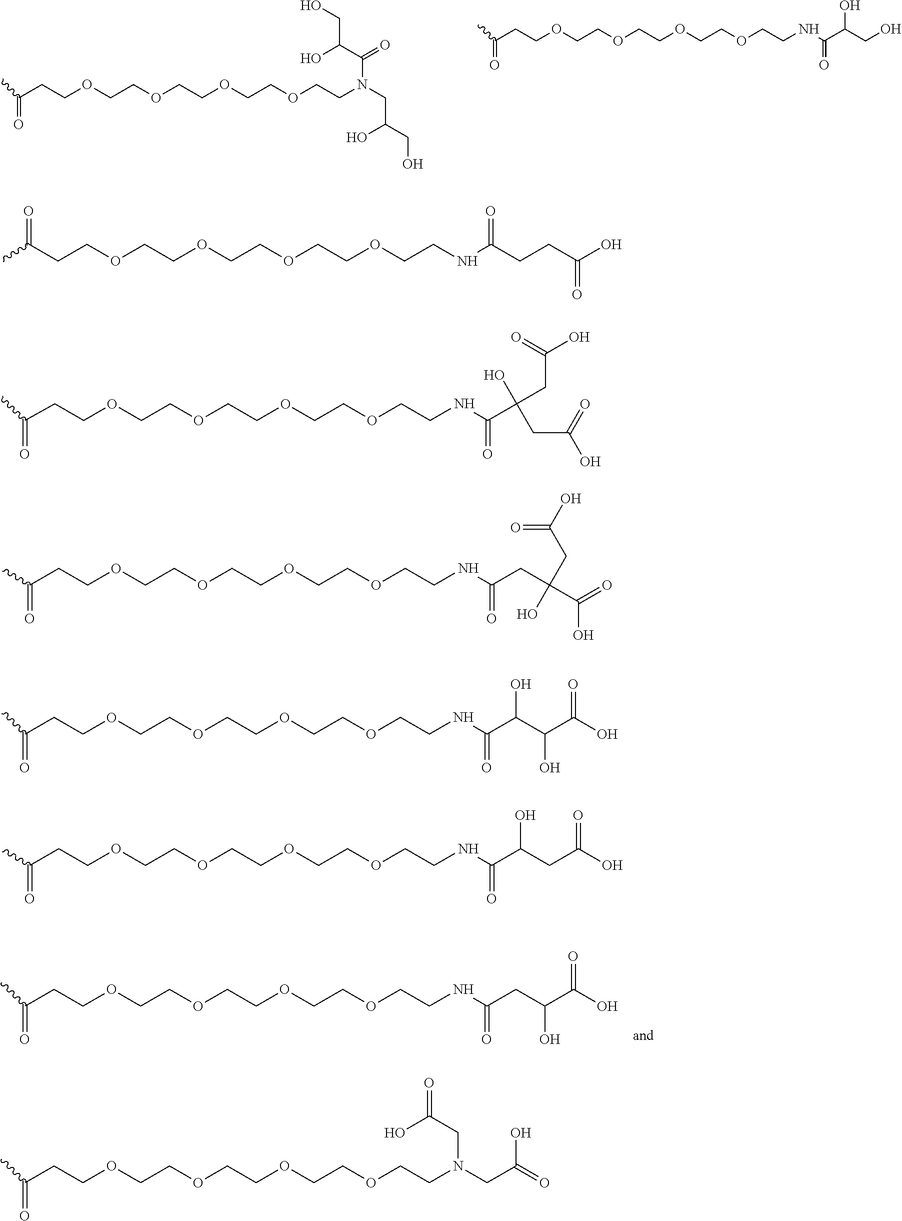

wherein the wavy line is an attachment site to R^{b}, or to the enzyme-cleavable group.

Embodiment 41. The linker compound of Embodiment 15, wherein R²⁴ and R²⁵ are independently selected from H and a chelator, wherein the chelator is optionally attached to the nitrogen of —NR²⁴R²⁵ by an alkylene, arylene, carbocyclo, heteroarylene, or heterocarbocylo; provided that both R²⁴ and R²⁵ are not H.

Embodiment 42. The linker compound of Embodiment 41, wherein the chelator is selected from ethylenediaminetetraacetic acid (EDTA), diethylenetriaminepentaacetic acid (DTPA), triethylenetetraminehexaacetic acid (TTHA), benzyl-DTPA, 1,4,7,10-tetraazacyclododecane-N,N′,N″,N′″-tetraacetic acid (DOTA), benzyl-DOTA, 1,4,7-triazacyclononane-N,N′,N″-triacetic acid (NOTA), benzyl-NOTA, 1,4,8,11-tetraazacyclotetradecane-1,4,8,11-tetraacetic acid (TETA) and N,N′-dialkyl substituted piperazine.

Embodiment 43. The linker compound of Embodiment 42, the polar group is selected from the following, or a stereoisomer or salt thereof:

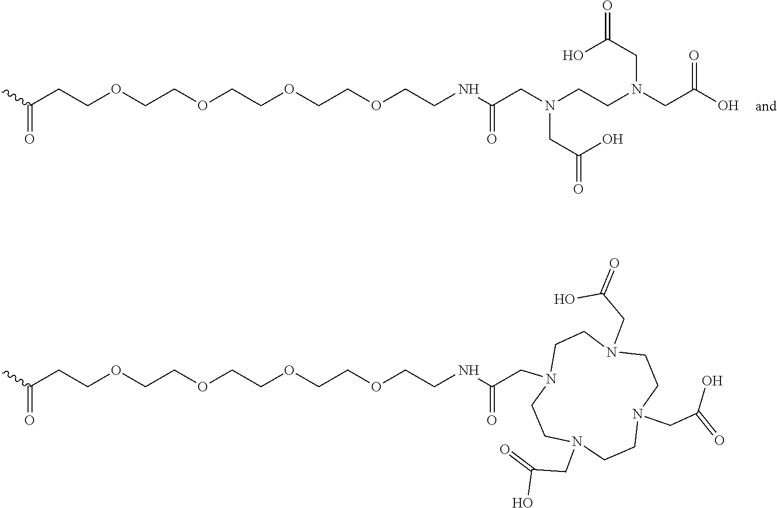

wherein the wavy line is an attachment site to R^{b}, or to the enzyme-cleavable group. PGP

Embodiment 44. The linker compound of Embodiment 15, wherein R²⁴ and R²⁵ are independently selected from a H, a polyhydroxyl-ether group, a substituted polyhydroxyl-ether group.

Embodiment 45. The linker compound of Embodiment 15, wherein —(NR²⁴R²⁵) is selected from the following, or a stereoisomer or salt thereof:

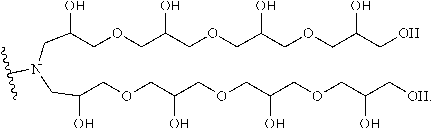 [SMILES: *N(CC(O)COCC(O)COCC(O)COCC(O)CO)CC(O)COCC(O)COCC(O)COCC(O)CO]

Embodiment 46. The linker compound of any one of Embodiments 13-32, wherein each monosaccharide is independently selected from: a C5 or C6 sugar selected from glucose, ribose, galactose, mannose, arabinose, 2-deoxyglucose, glyceraldehyde, erythrose, threose, xylose, lyxose, allose, altrose, gulose, idose talose, aldose, and ketose; a sugar acid selected from gluconic acid, aldonic acid, uronic acid and ulosonic acid; or an amino sugar selected from glucosamine, N-acetyl glucosamine, galactosamine, and N-acetyl galactosamine.

Embodiment 47. The linker compound of any one of Embodiments 1 to 9, wherein the attachment site β is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 48. The linker compound of any one of Embodiments 1 to 14, the polar group has a formula selected from the following:

(a) ˜R²⁰—R²¹—[O—CH₂—CH₂]ₙ₂₀—R²²—R³⁰ (XXX)
or a stereoisomer or salt thereof, wherein:
R²⁰ is an attachment group to site β or to site R^{b}, or to the enzyme-cleavable group;
R²¹ and R²² are each independently, a bond or C₁-C₃ alkylene groups;
R³⁰ is selected from an optionally substituted C₃-C₁₀ carbocycle; thiourea; optionally substituted thiourea; urea; optionally substituted urea; sulfamide; alkyl sulfamide; acyl sulfamide, optionally substituted alkyl sulfamide; optionally substituted acyl sulfamide; sulfonamide; optionally substituted sulfonamide; guanidine, including alkyl and aryl guanidine; phosphoramide; or optionally substituted phosphoramide; or R³⁰ is selected from azido, alkynyl, substituted alkynyl, —NH—C(O)-alkynyl, —NH—C(O)-alkynyl-R⁶⁵; cyclooctyne; —NH-cyclooctyne, —NH—C(O)-cyclooctyne, or —NH-(cyclooctyne)₂; wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(b) ˜R²⁰—R²¹—[O—CH₂—CH₂]ₙ₂₀—R²²—NH—C(O)—R³¹ (XXXI)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(c) ˜-R²⁰—R²¹—[O—CH₂—CH₂]ₙ₂₀—R²²—C(O)NH—R³¹ (XXXII)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch, independently, having 1 to 26 ethylene glycol subunits and each branch having an R³⁵ at its terminus;
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle and optionally substituted heteroaryl; and
n20 is 2 to 26;

(d) ˜R²⁰—R²¹—[O—CH₂—CH₂]ₙ₂₀—R²²—C(O)NR³¹—R²²—NR²⁴R²⁵ (XXXIII)
or a stereoisomer or salt thereof, wherein:
R²⁰ is an attachment group to site β or to site R^{b}, or to the enzyme-cleavable group;
R³¹ is H or R²²—NR²⁴R²⁵;
R²¹ and R²² are each, independently, a bond or C₁-C₃ alkylene groups;
R²⁴ and R²⁵ are each independently selected from a H; polyhydroxyl group; substituted polyhydroxyl group; —C(O)-polyhydroxyl group; substituted —C(O)— polyhydroxyl group, provided that R²⁴ and R²⁵ are not both H; and
n20 is 2 to 26;

(e) ˜R²⁰—R²¹—[O—CH₂—CH₂]ₙ₂₀—R²²—N(R³³—R³¹)₂ (XXXIV)
or a stereoisomer or salt thereof, wherein:
R²⁰ is an attachment group to site β or to site R^{b}, or to the enzyme-cleavable group;
R²¹ and R²² are each, independently, a bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, C₁-C₃ alkylene-C(O), —C(O)—C₁-C₃ alkylene, or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and
n20 is 2 to 26;

(f) ˜R²⁰—(R²¹—[CH₂—CH(OR³⁴)—CH₂—O]ₙ₂₀—R³⁶)ₙ₂₅ (XXXV)
or a stereoisomer or salt thereof, wherein:
R²⁰ is an attachment group to site β or to site R^{b}, or to the enzyme-cleavable group;
each R²¹ is independently a bond, —O— or C₁-C₃ alkylene group;
each R³⁴ is independently H, —[CH₂—CH(OH)—CH₂—O]ₙ₂₀—R³⁶, —C(O)—NR²⁴R²⁵ or —C(O)N(R^{N})—C₁-C₆alkylene-NR²⁴R²⁵;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)—NR²⁴R²⁵, —C(O)N(R^{N})_C₁-C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)— polyhydroxyl group; provided that both R⁴⁴ and R⁴⁵ are not H;
each n20 is independently 1 to 26; and
n25 is 1 or 2;

(g) ˜R²⁰—R²¹—[[CH₂—CH₂—O]ₙ₂₀—R²²—[CH₂—[CH(OH)]ₙ₂₃—CH₂—O]ₙ₂₁]ₙ₂₂—R²³—NR²⁴—R²⁵ (XXXVI)
or a stereoisomer or salt thereof, wherein:
R²⁰ is an attachment group to site β or to site R^{b}, or to the enzyme-cleavable group;
R²¹, R²² and R²³ are each independently a bond or C₁-C₃ alkylene group;
R²⁴ and R²⁵ are each independently selected from a H; polyhydroxyl group; substituted polyhydroxyl group; —C(O)-polyhydroxyl group; substituted —C(O)— polyhydroxyl group, provided that R²⁴ and R²⁵ are not both H;
each n20 is independently 0 to 26, and each n21 is independently 0 to 26, with the proviso that at least one of n20 or n21 is 2 to 26;
n22 is 1 to 5;
each n23 is independently 1 or 2;

(h) ˜R²⁰—(R²¹—[O—CH₂—CH₂]ₙ₂₀—R²²—N(R^{N})—CO₂—[CH₂—CH(OR³⁴)—CH₂—O]ₙ₂₁—R³⁶)ₙ₂₅ (XXXVII)
or a stereoisomer or salt thereof, wherein:
R²⁰ is an attachment group to site β or to site R^{b}, or to the enzyme-cleavable group;
R²¹ and R²² are each independently a bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that both R²⁴ and R²⁵ are not H;
each R³⁴ is independently H, —[CH₂—CH(OH)—CH₂—O]ₙ₂₀—R³⁶ or —C(O)N(R^{N})—C₁-C₆alkylene-NR²⁴R²⁵;
each R³⁶ is independently H, C₁-C₆alkylene-C(OH)H—NR⁴⁴R⁴⁵, C₁-C₆alkylene-C(OH)H—C₁-C₆alkylene-NR⁴⁴R⁴⁵, —C(O)N(R^{N})—C₁_C₆alkylene-NR²⁴R²⁵, C₁-C₆alkylene-C(O)NR²⁴R²⁵ or C₁-C₆alkylene-CO₂R³⁷;
each R³⁷ is independently H or C₁-C₆ alkyl;
R⁴⁴ and R⁴⁵ are each independently selected from a H; polyhydroxyl group; substituted polyhydroxyl group; —C(O)-polyhydroxyl group; substituted —C(O)— polyhydroxyl group; provided that both R⁴⁴ and R⁴⁵ are not H;
n20 is 2 to 26;
n21 is 1 to 26; and
n25 is 1 or 2;

(i) ˜R²⁰—(R²¹—[N(R^{N})—C(O)—[O—CH₂—CH(OH)—CH₂]ₙ₂₀]ₙ₂₁—R²²—NR²⁴R²⁵)ₙ₂₅ (XXXVIII)
or a stereoisomer or salt thereof, wherein:
R²⁰ is an attachment group to site β or to site R^{b}, or to the enzyme-cleavable group;
R²¹ and R²² are each independently bond or C₁-C₃ alkylene groups;
R^{N} is H or C₁-C₄alkyl;
R²⁴ and R²⁵ are each independently selected from a H; polyhydroxyl group; or substituted polyhydroxyl group, provided that R²⁴ and R²⁵ are not both H;
n20 is 2 to 26;
n21 is 1 to 4; and
n25 is 1, 2 or 3;

(j) ˜R²⁰—(R²¹—[C(R^{α})H—C(O)—N(R^{N})]ₙ₂₀—R²²—[CH₂—CH₂—O]ₙ₂₀—NR²⁵)ₙ₂₅ (XXXIX)
or a stereoisomer or salt thereof, wherein:
R²⁰ is an attachment group to site β or to site R^{b}, or to the enzyme-cleavable group;
R²¹ and R²² are each, independently, a bond, C₁-C₃ alkylene, —C₁-C₅alkylene-[O—CH₂—CH₂-]ₙ₂₀, —[CH₂—CH₂—O]ₙ₂₀—C₁-C₅alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, wherein R²⁸ is the sugar unit of formula (XII) or (XIII); or —NR²⁴R²⁵ together from a C₃-C₈ heterocycle), provided that R²⁴ and R²⁵ are not both H;
each n20 is independently 0 to 26, with the proviso that at least one n20 is 2 to 26; and
n25 is 1 or 2; or

(k) ˜R²⁰—R²¹—[C(R^{α})H—C(O)—N(R^{N})]ₙ₂₀—R²²—[CH₂—CH₂—O]ₙ₂₀—NR²⁴R²⁵|R²¹—[C(R^{α})H—C(O)—N(R^{N})]ₙ₂₁—R²²—[CH₂—CH₂—O]ₙ₂₁—R²³—CO₂—R²⁶ (XXXVX)
or a stereoisomer or salt thereof, wherein:
R²⁰ is an attachment group to site β or to site R^{b}, or to the enzyme-cleavable group;
R²¹, R²² and R²³ are each, independently, a bond, C₁-C₃ alkylene, —C₁-C₃alkylene-[O—CH₂—CH₂-]ₙ₂₀, —[CH₂—CH₂—O]ₙ₂₀—C₁-C₃alkylene- or —C₁-C₃alkylene-[O—CH₂—CH₂—]ₙ₂₀—C(O)—;
each R^{α} is independently H or —R²²—NR²⁴R²⁵;
each R^{N} is independently H, C₁-C₆ alkyl or —R²²—NR²⁴R²⁵;
R²⁴ and R²⁵ are each independently selected from a H; polyhydroxyl group; substituted polyhydroxyl group; —C(O)-polyhydroxyl group; substituted —C(O)— polyhydroxyl group; optionally substituted C₃-C₁₀ carbocycle; optionally substituted C₁-C₃ alkylene C₃-C₁₀ carbocycle; optionally substituted heteroaryl; optionally substituted carbocycle; substituted —C₁-C₈ alkyl; substituted —C(O)—C₁-C₈ alkyl; a chelator; —C(O)—R²⁸, where R²⁸ is the sugar unit of formula (XII) or (XIII); or —NR²⁴R²⁵ together from a C₃-C₈ heterocycle), provided that R²⁴ and R²⁵ are not both H;
R²⁶ is H or C₁-C₆ alkyl;
each n20 is independently 0 to 26, with the proviso that at least one n20 is 2 to 26; and
each n21 is independently 0 to 26, with the proviso that at least one n21 is 2 to 26.

Embodiment 49. The linker compound of any one of Embodiments 1 to 14, the polar group has a formula selected from the following, or a stereoisomer or salt thereof:

˜R²⁰—R²¹—[O—CH₂—CH₂]ₙ₂₀—R²²—NH—C(O)—R³¹ (XXXI),

˜R²⁰—R²¹—[O—CH₂—CH₂]_{n2O}—R²²—C(O)NH—R³¹ (XXXII), and

˜R²⁰—R²¹—[O—CH₂—CH₂]_{n2O}—R²²—N—(R³³—R³¹)₂ (XXXIII);

wherein:
R²⁰ is an attachment group to site β or to site R^{b}, or to the enzyme-cleavable group;
R²¹ and R²² are each, independently, bond or C₁-C₃ alkylene groups;
R³¹ is a branched polyethylene glycol chain, each branch having 1 to 26 ethylene glycol subunits and each branch having an R³⁵ at its terminus;
R³³ is C₁-C₃ alkylene, —C₁-C₃ alkylene-C(O), —C(O)—C₁-C₃ alkylene or —C(O)—C₁-C₃ alkylene-C(O);
R³⁵ is azido, alkynyl, alkynyl-R⁶⁵, cyclooctyne or cyclooctyne-R⁶⁵, wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; the wavy (˜) line indicates an attachment site to R²⁰; and n20 is 2 to 26.

Embodiment 50. The linker compound of Embodiment 48 or 49, the polar group is formed from a precursor group selected from the following:

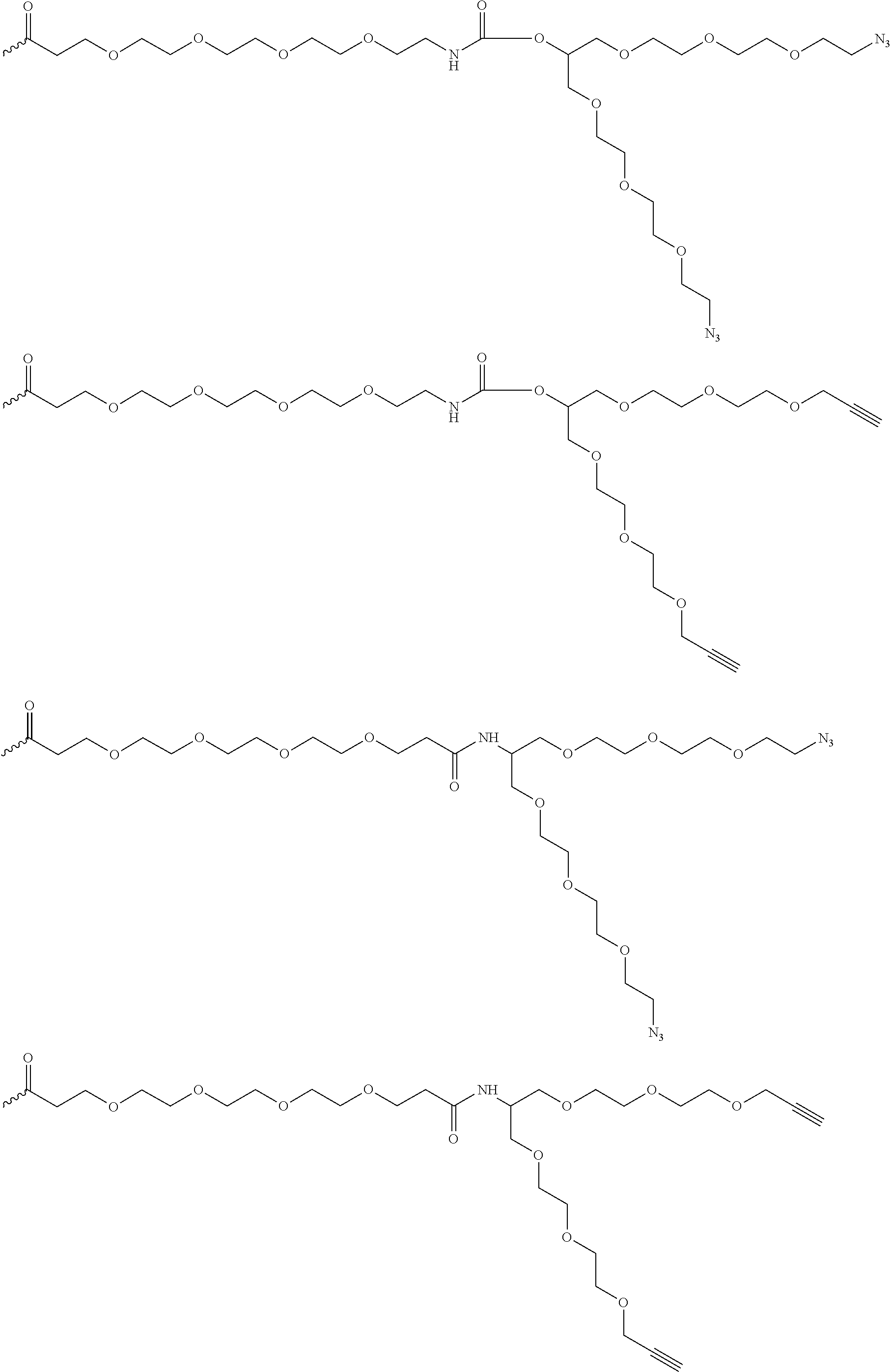

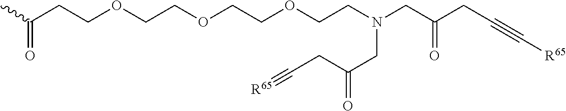

wherein R⁶⁵ is selected from optionally substituted alkyl, optionally substituted alkenyl, optionally substituted alkynyl, optionally substituted carbocycle, optionally substituted aryl, optionally substituted heterocarbocycle or optionally substituted heteroaryl; and the wavy line is an attachment site to R^{b}, or to the enzyme-cleavable group.

Embodiment 51. The linker compound of of Embodiment 48 or 49, wherein the attachment to site □ to site R^{b}, or to the enzyme-cleavable group is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, and protected forms thereof.

Embodiment 52. The linker compound of any one of Embodiments 1-14, the polar group has a formula:

˜R²⁰—(R⁴³—R⁴¹—[O—CH₂—CH₂]ₙ₄₀—R⁴²—R⁴³—(NR⁴⁴R⁴⁵)ₙ₄₁)ₙ₄₂ (XL)
or a stereoisomer or salt thereof, wherein:
R²⁰ is an attachment group to site □, to site R^{b}, or to the enzyme-cleavable group;
R⁴¹ and R⁴² are each, independently, bond or C₁-C₆ alkylene;
each R⁴³ is, independently, a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁—C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 2 to 26;
n41 is 1 to 6; and
n42 is 1 to 6.

Embodiment 53. The linker compound of any one of Embodiments 1-14, the polar group has a formula:

˜R²⁰—(R⁴¹—[O—CH₂—CH₂]ₙ₄₀—R⁴²—R⁴³—(NR⁴⁴R⁴⁵)ₙ₄₁)ₙ₄₂ (XLI)
or a stereoisomer or salt thereof, wherein:
R²⁰ is an attachment group to site □, to site R^{b}, or to the enzyme-cleavable group;
R⁴¹ and R⁴² are each, independently, bond or C₁-C₆ alkylene;
R⁴³ is a bond or is selected from C₁-C₁₂ alkylene, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, C(O)—NH—C₁-C₁₂ alkylene, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 26;
n41 is 1 to 6; and
n42 is 1 to 6.

Embodiment 54. The linker compound of any one of Embodiments 1-14, the polar group has a formula:

˜R²⁰—(R⁴¹—[O—CH₂—CH₂]ₙ₄₀—R⁴²—R⁴³—(NR⁴⁴R⁴⁵)ₙ₄₁)ₙ₄₂ (XLII)
or a stereoisomer or salt thereof, wherein:
R²⁰ is an attachment group to site □, to site R^{b}, or to the enzyme-cleavable group;
R⁴¹ and R⁴² are each, independently, bond or C₁-C₃ alkylene;
R⁴³ is a bond or is selected from C₁-C₆ alkylene, —NH—C₁-C₁₂ alkylene, —C₁-C₆ alkylene-NH—, —C(O)—C₁-C₆ alkylene, —C₁-C₆ alkylene-C(O)—, —NH—C₁-C₆ alkylene-C(O)—, —C(O)—C₁-C₆ alkylene-NH—, —NH—C(O)—NH—, —NH—C(O)—, —NH—C(O)—C₁-C₆ alkylene, —C(O)—NH—C₁-C₁₂ alkylene, -heteroarylene, heteroaryl-C₁-C₆ alkylene, heteroaryl-C₁-C₆ alkylene-C(O)—, or —C(O)NR⁴⁶R⁴⁷, wherein one of R⁴⁶ and R⁴⁷ is H or C₁-C₆ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate; n40 is 1 to 16;
n41 is 1 to 4; and
n42 is 1 to 4.

Embodiment 55. The linker compound of any one of Embodiments 15, 48, 49, and 52-54 wherein R²⁰ is formed from a functional group of a precursor compound of the polar group, said functional group selected from halo, aldehyde, carboxyl, amino, alkynyl, azido, hydroxyl, carbonyl, carbamate, thiol, urea, thiocarbamate, thiourea, sulfonamide, acyl sulfonamide, alkyl sulfonate, triazole, azadibenzocyclooctyne, hydrazine, carbonylalkylheteroaryl, or protected forms thereof.

Embodiment 56. The linker compound of any one of Embodiments 15, 48, 19, and 52-54, wherein R²⁰ comprises one of the following structures:

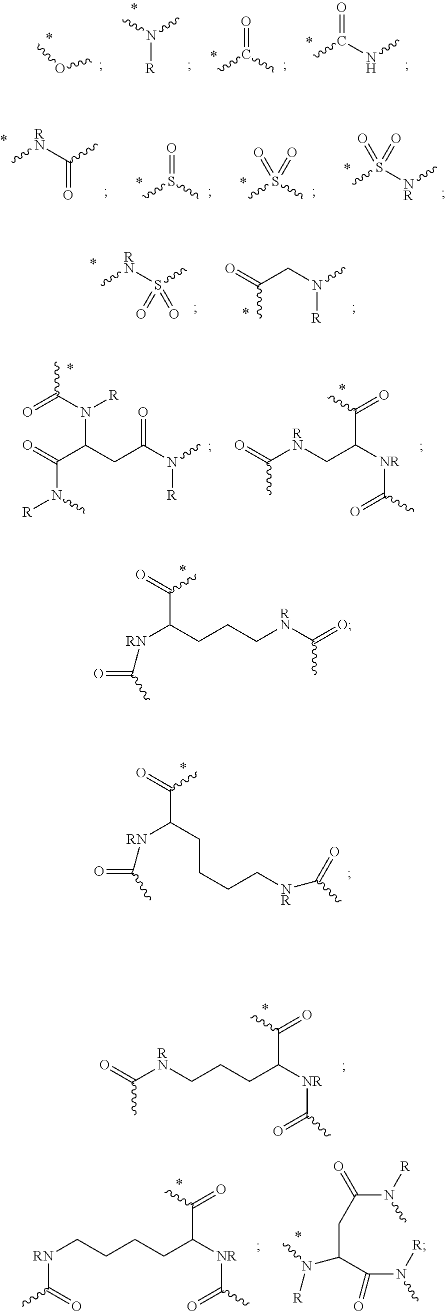

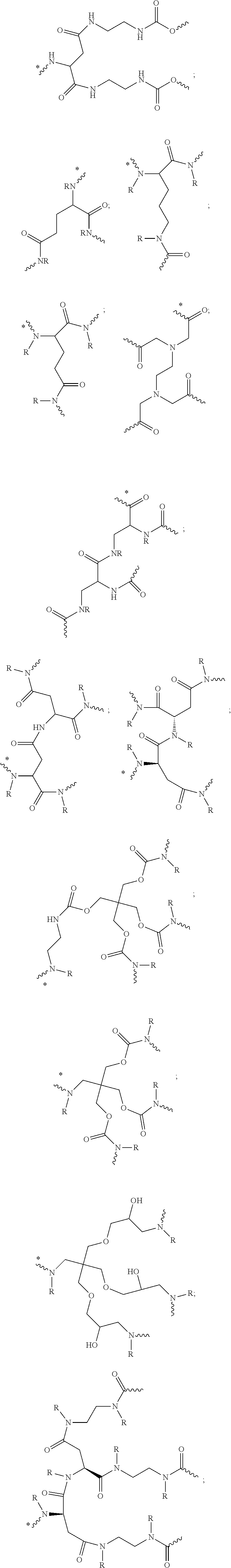

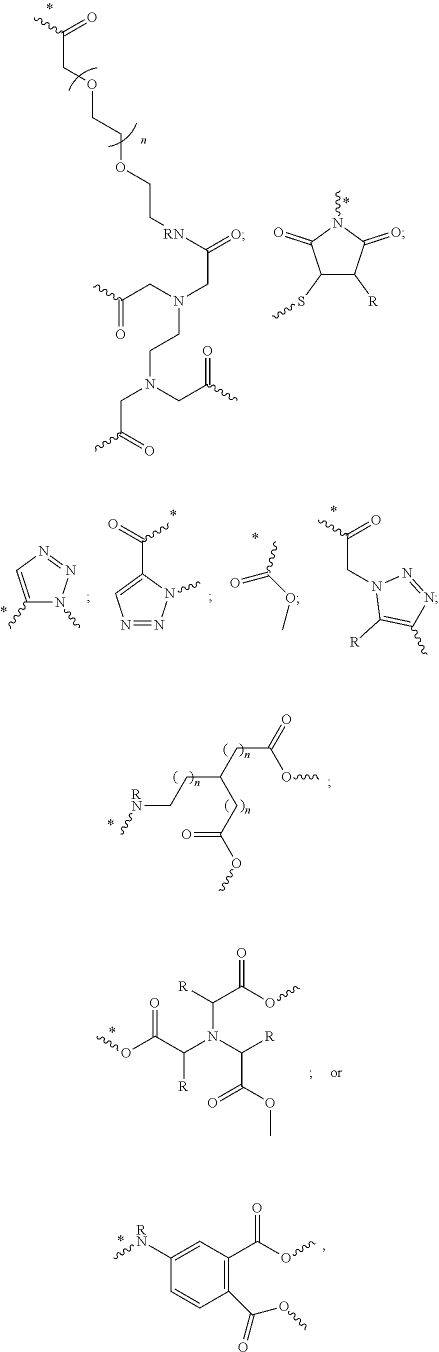

or a stereoisomer thereof, wherein R is H, C₁-C₆ alkyl or polyhydroxyl group, n is 0 to 12, the ( [symbol] ) indicates an attachment to site β or to site R^{b}, or to the enzyme-cleavable group, and the ( [symbol] ) indicates an attachment site to a remainder portion of the polar group.

Embodiment 57. The compound of any one of Embodiments 15, 48, 19, and 52-54, wherein R²⁰ has one of the following structures:

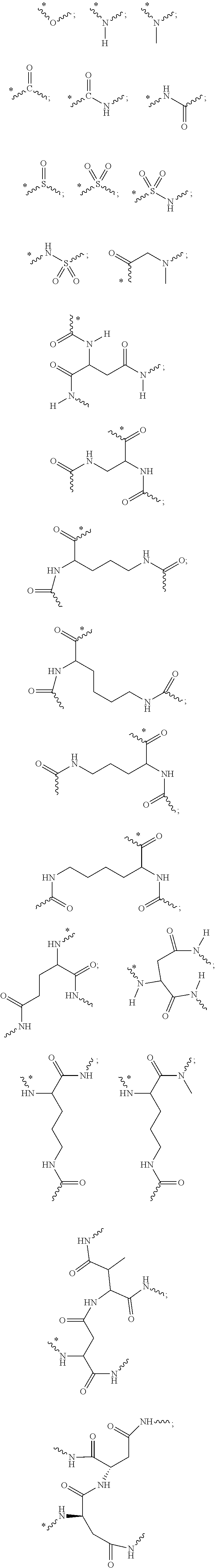

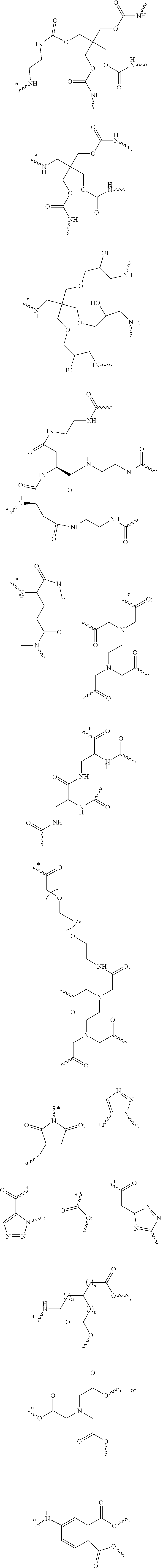

or a stereoisomer thereof, wherein n=0 to 12, the ( [symbol] ) indicates an attachment to site β or to site R^{b}, or to the enzyme-cleavable group, and the ( [symbol] ) indicates an attachment site to a remainder portion of the polar group.

Embodiment 58. The linker compound of any one of Embodiments 52-57, wherein R⁴³—(NR⁴⁴R⁴⁵)ₙ₄₁ has one of the following structures:

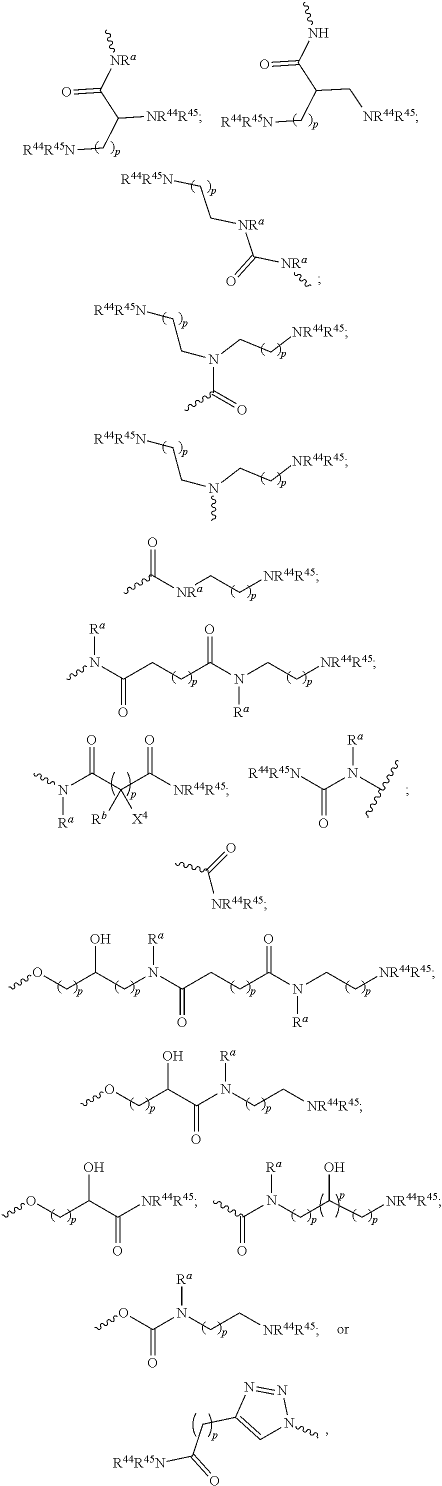

or a stereoisomer thereof, wherein R═H, C₁-C₆ alkyl, a polyhydroxyl group, or a substituted polyhydroxyl group; and the ( [symbol] ) indicates the attachment site of R⁴³ to the remainder of the polar group.

[symbol]

Embodiment 59. The linker compound of any one of Embodiments 52-57, wherein R⁴³—(NR⁴⁴R⁴⁵)ₙ₄₁ has one of the following structures:

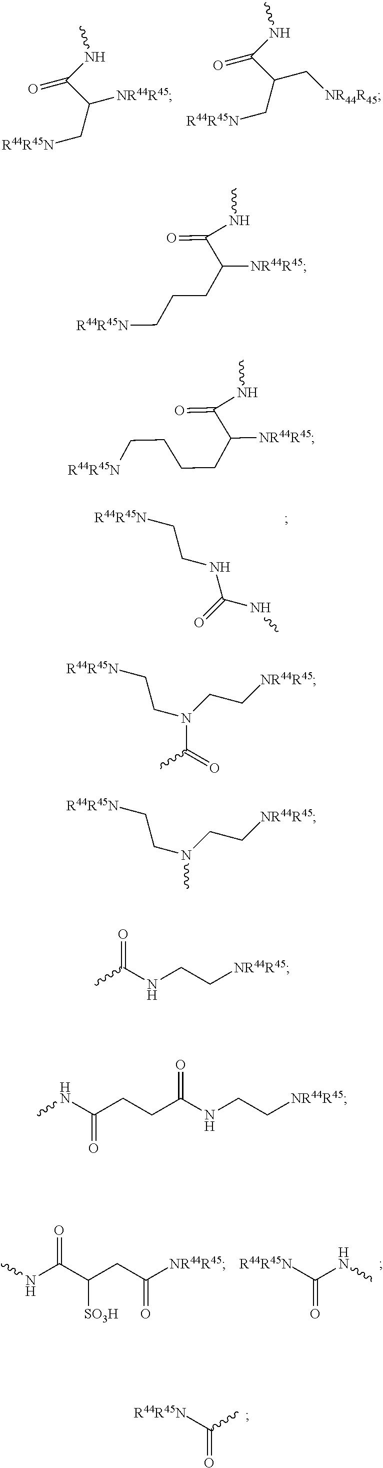

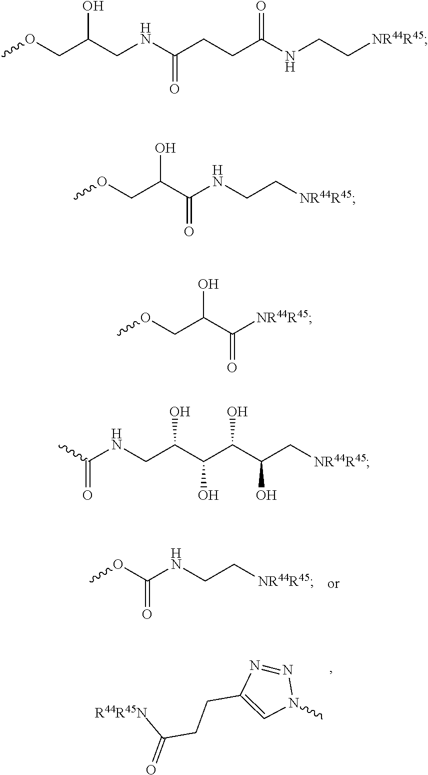

or a stereoisomer thereof, wherein the ( [symbol] ) indicates the attachment site of R⁴³ to the [symbol] remainder of the polar group.

Embodiment 60. The linker compound of any one of Embodiments 52-59, wherein —NR⁴⁴R⁴⁵ has one of the following structures:

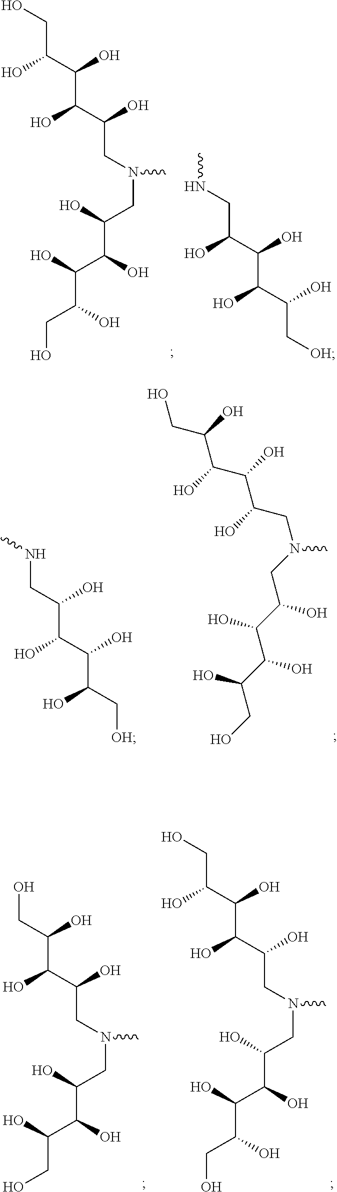

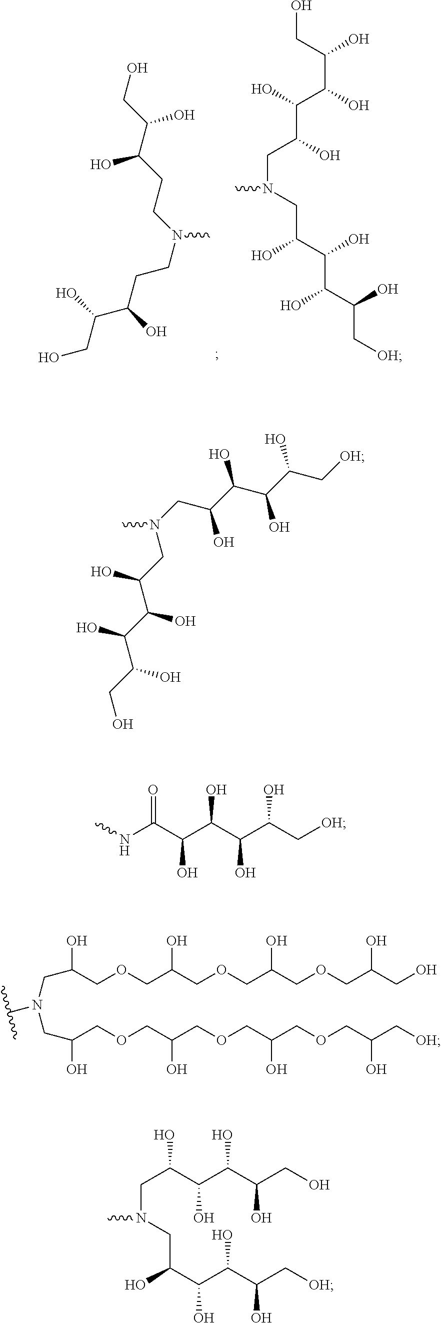

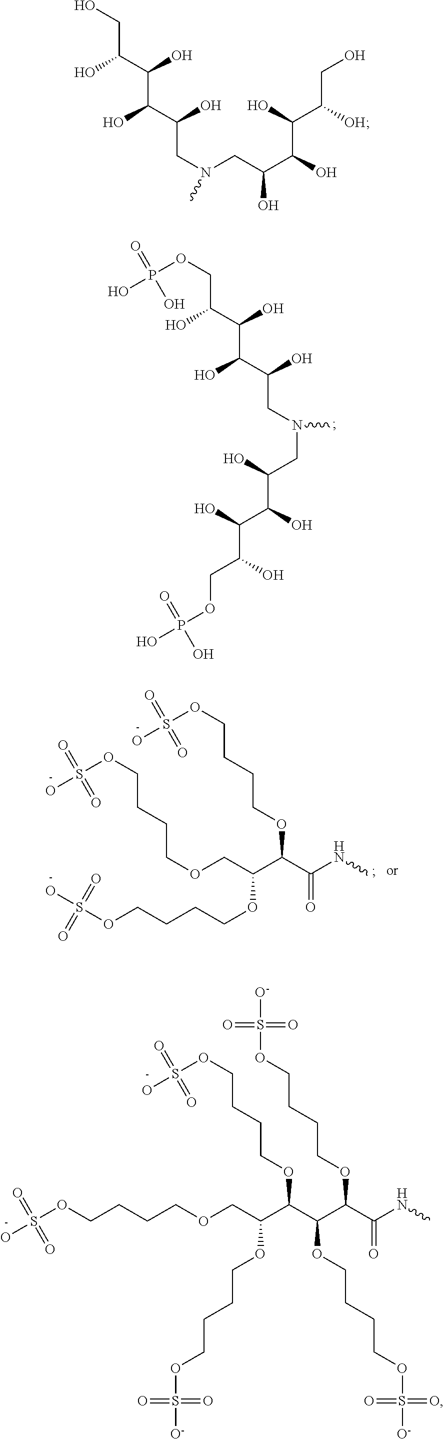

or a stereoisomer thereof, wherein the ( [symbol] ) indicates the attachment site of —NR⁴⁴R⁴⁵ to the remainder of the polar group.

[symbol]

Embodiment 61. The linker compound of any one of Embodiments 1-60, the polar group has one of the following structures prior to attachment to the linker unit:

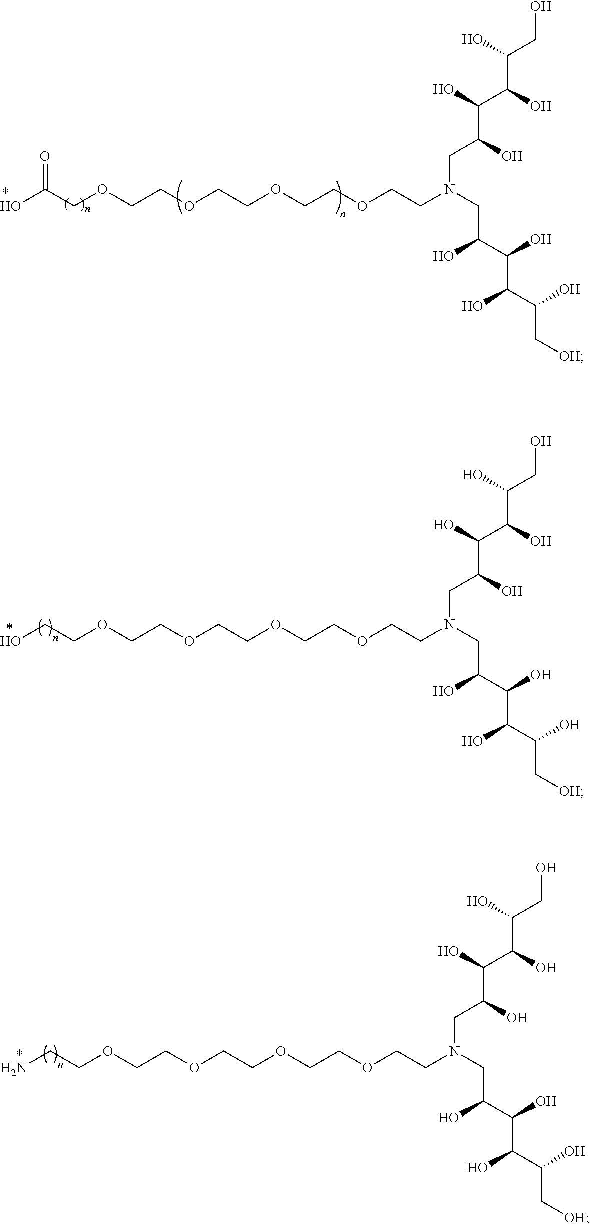

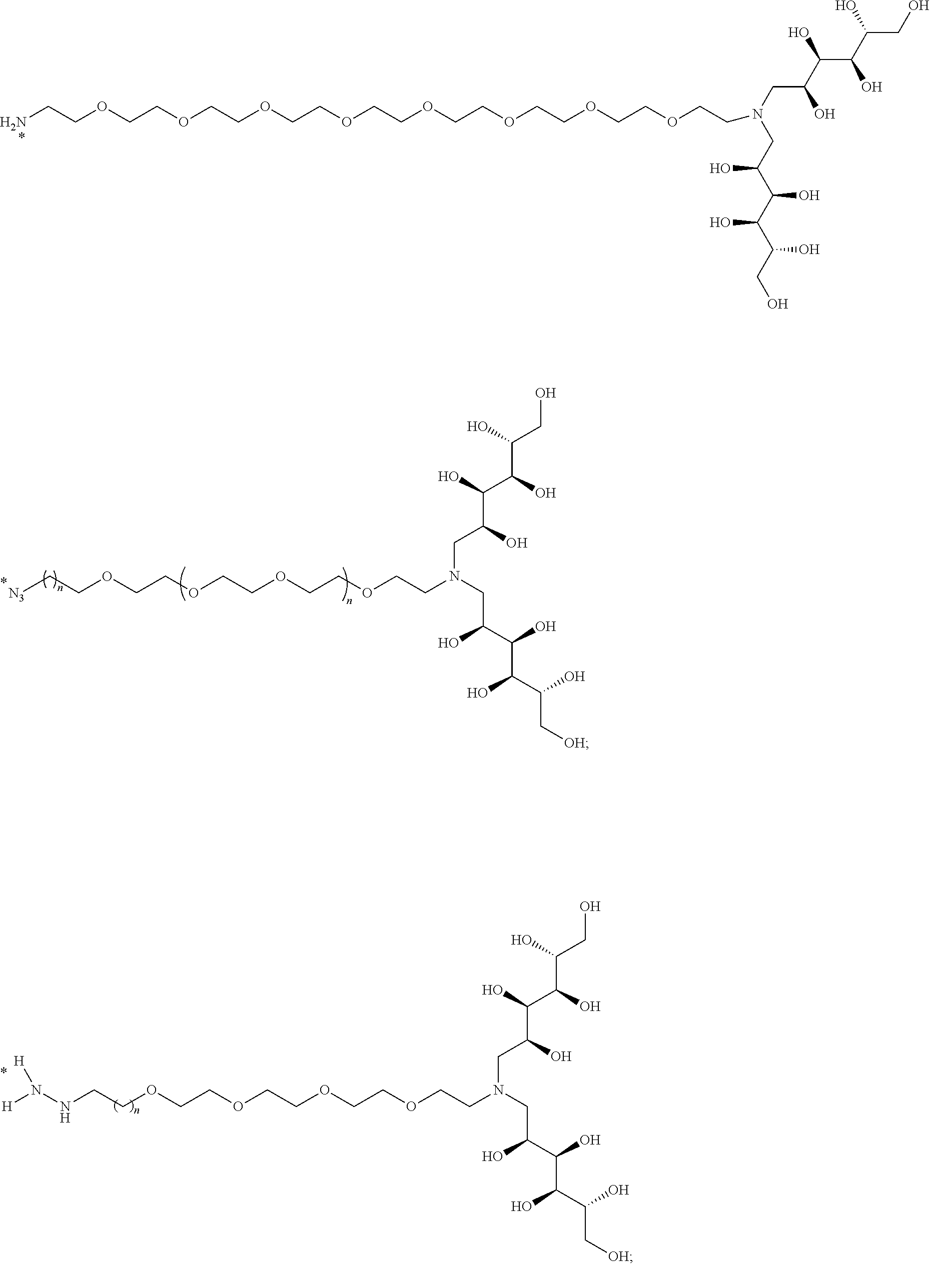

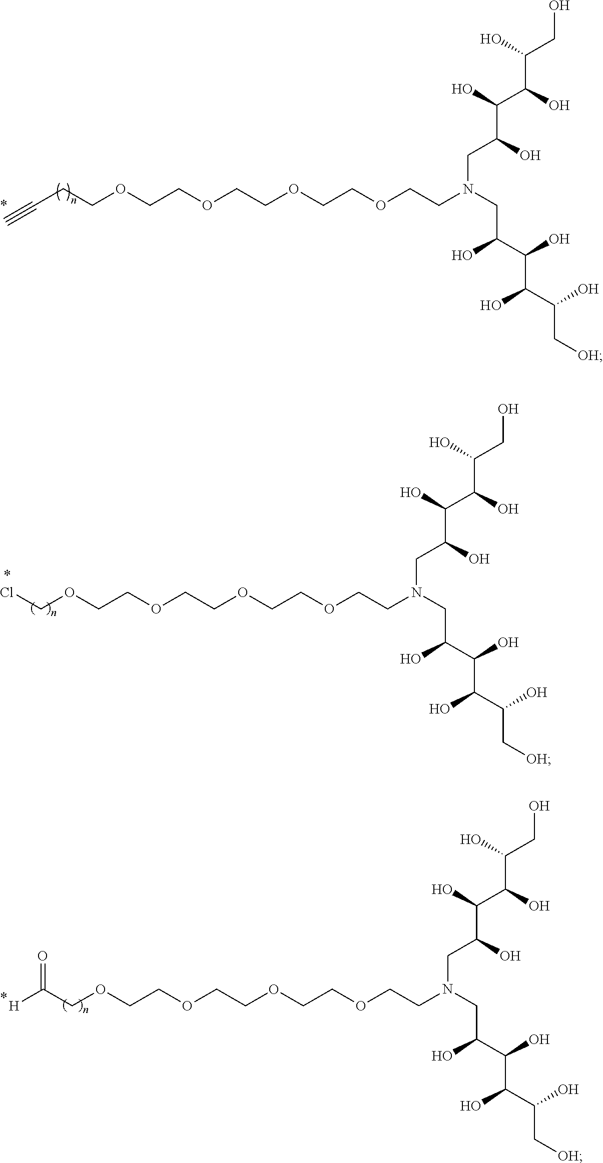

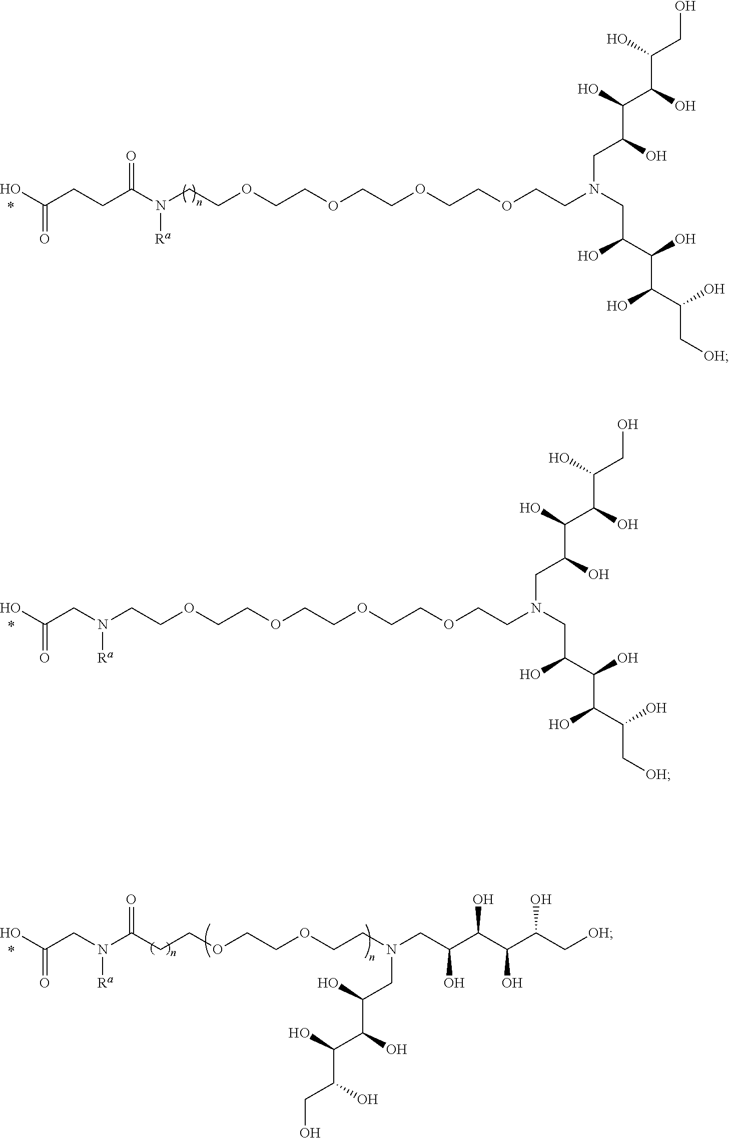

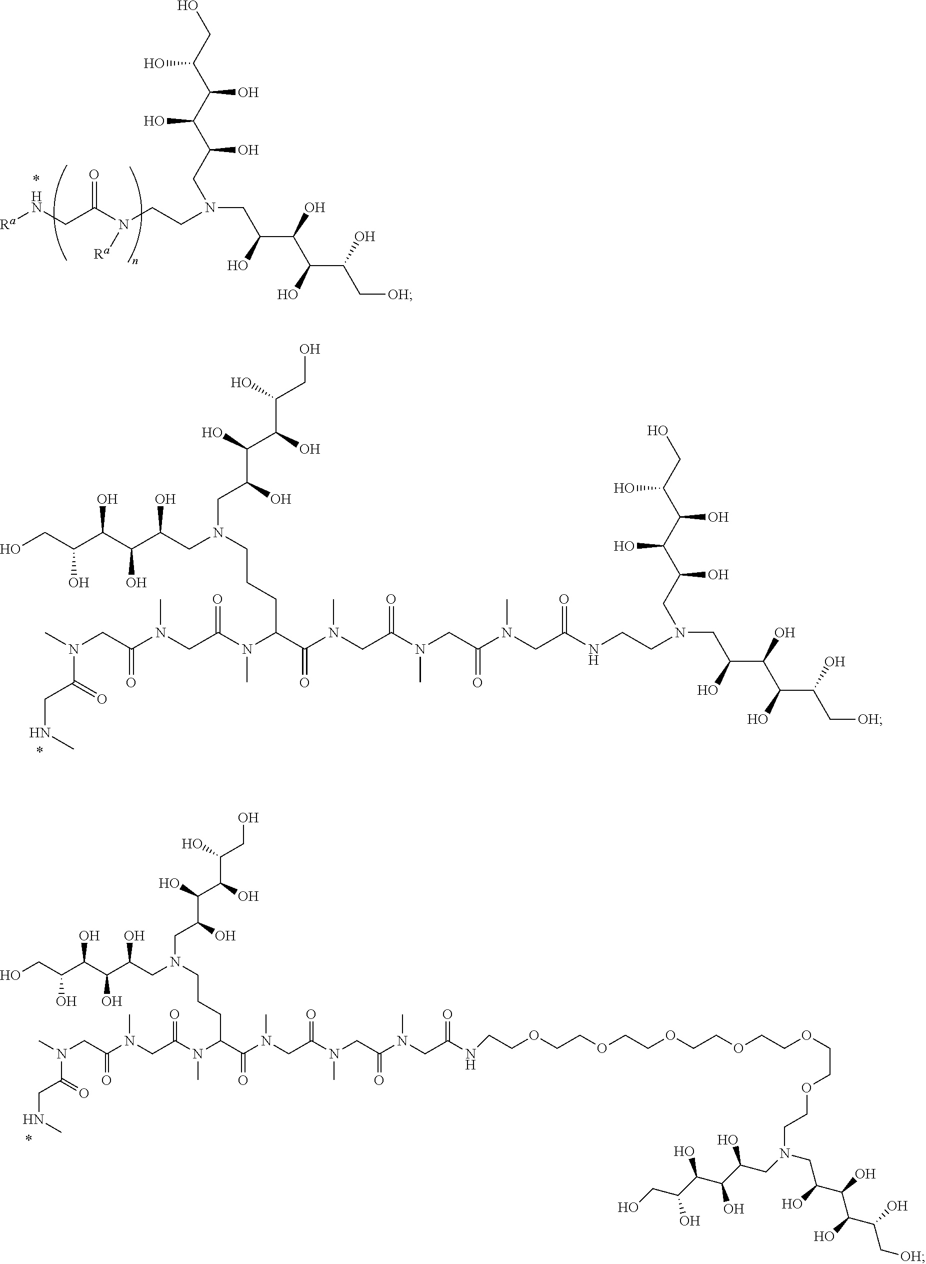

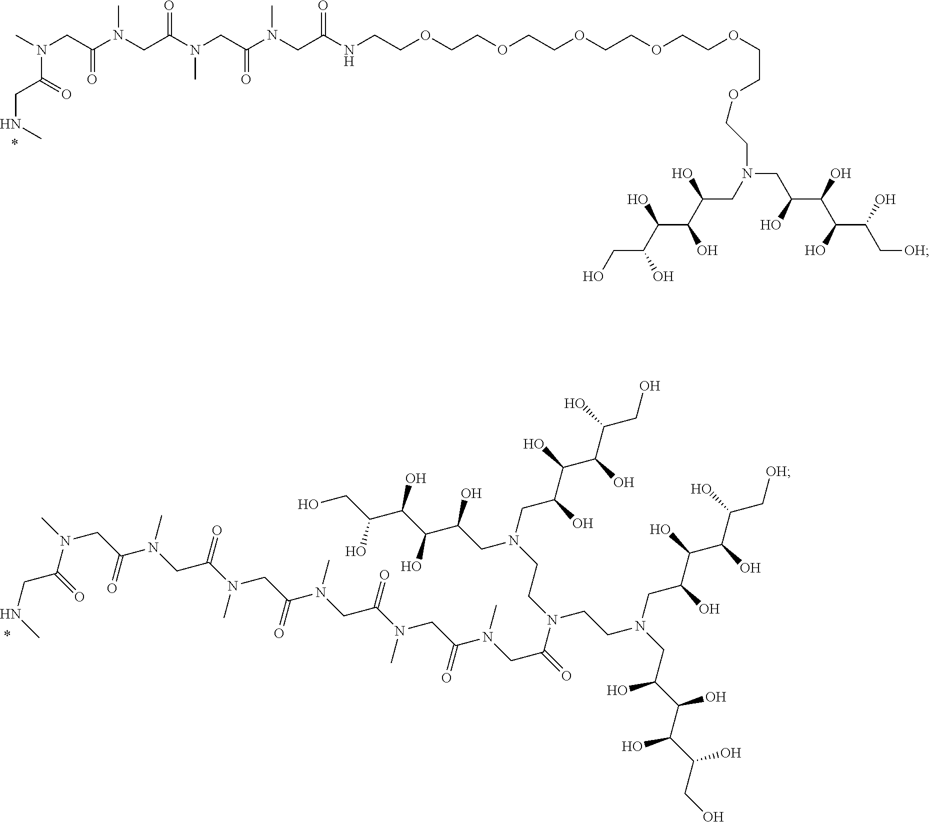

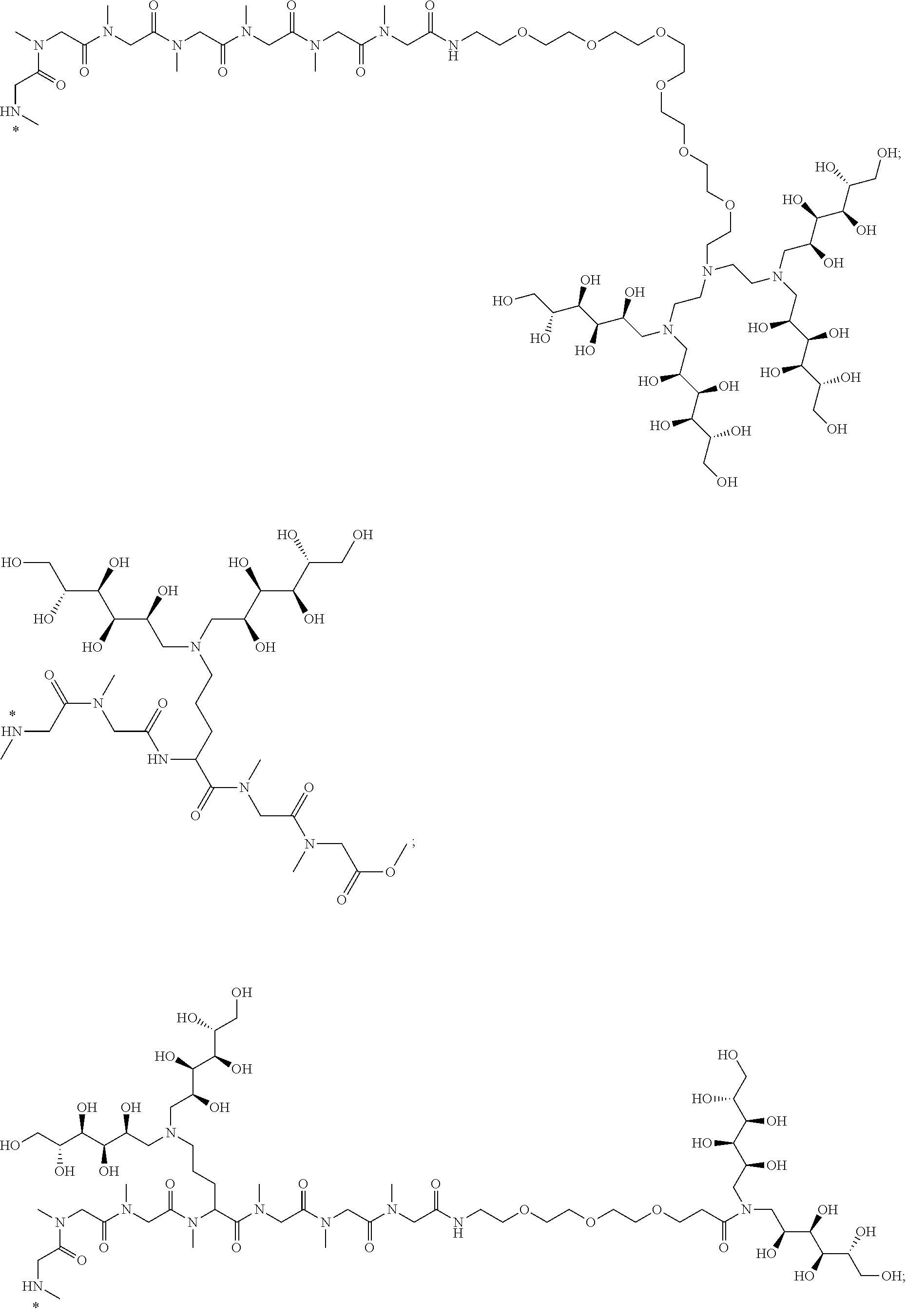

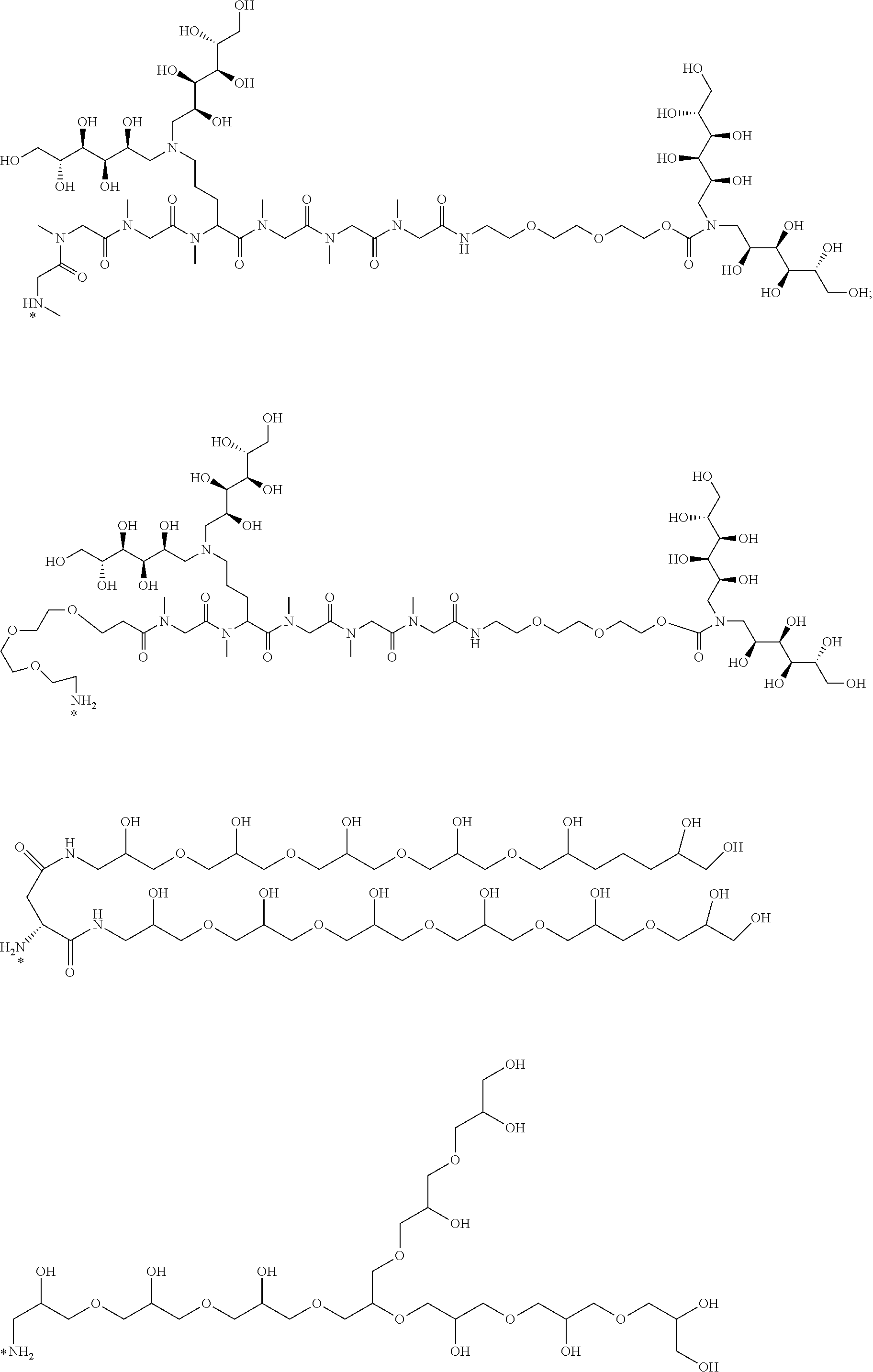

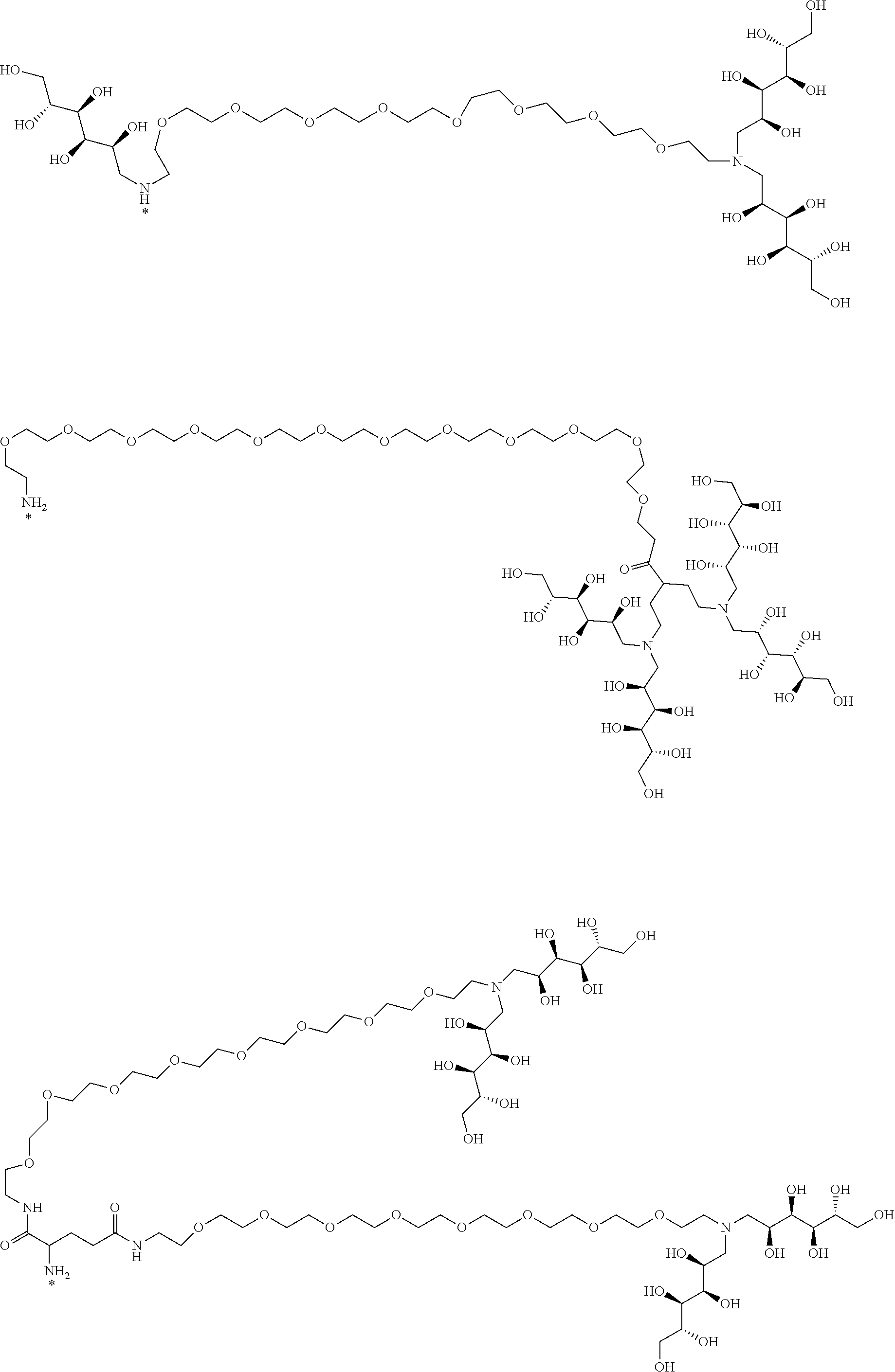

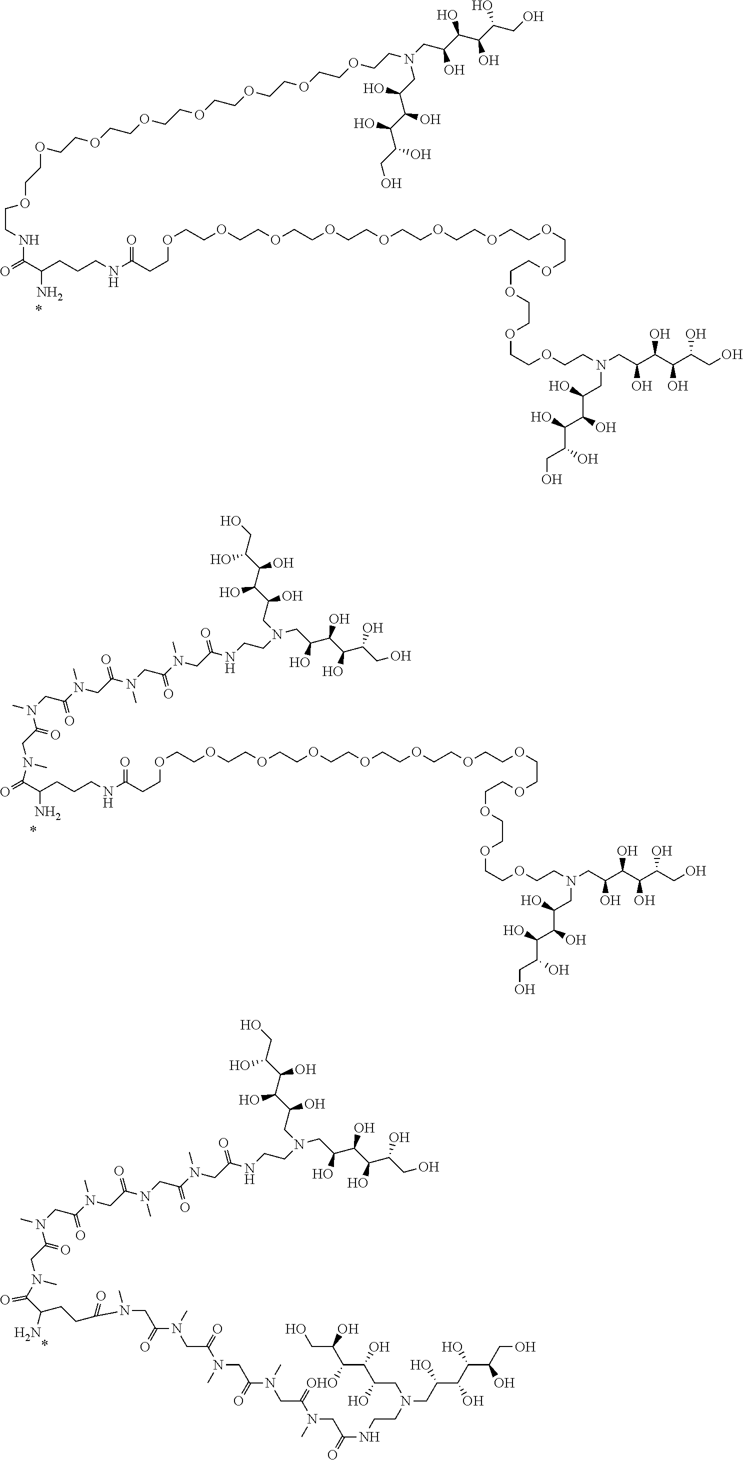

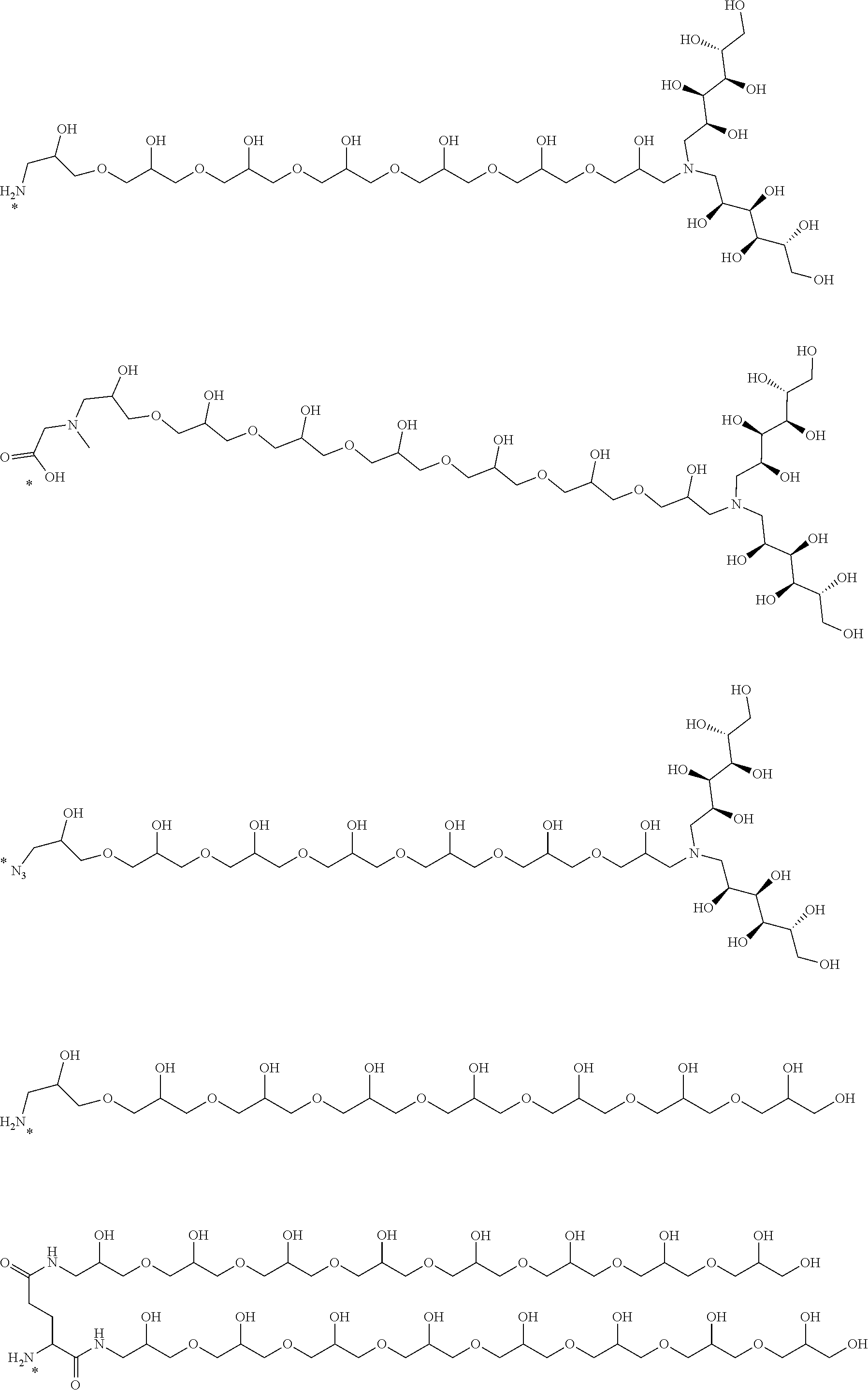

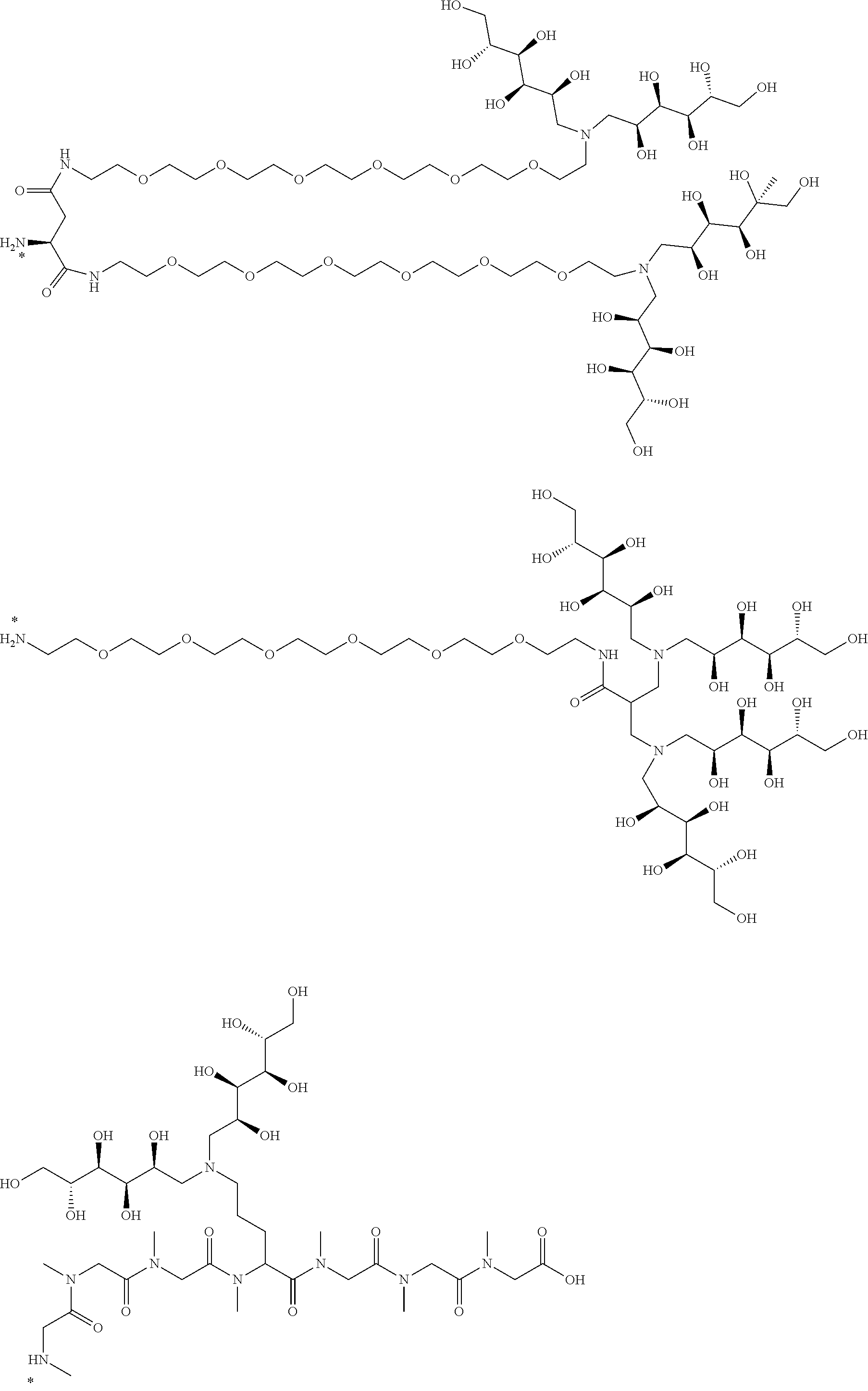

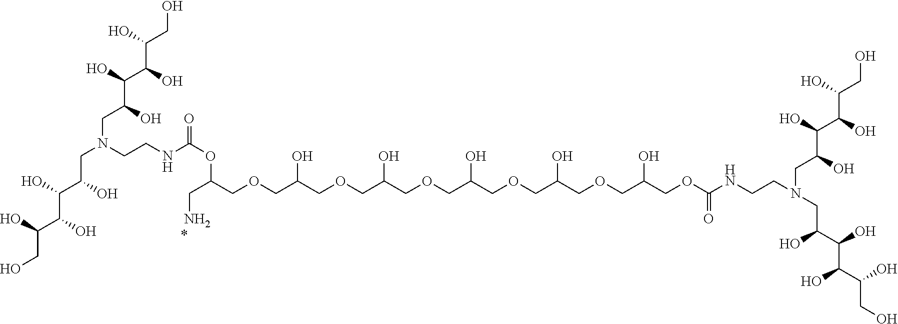

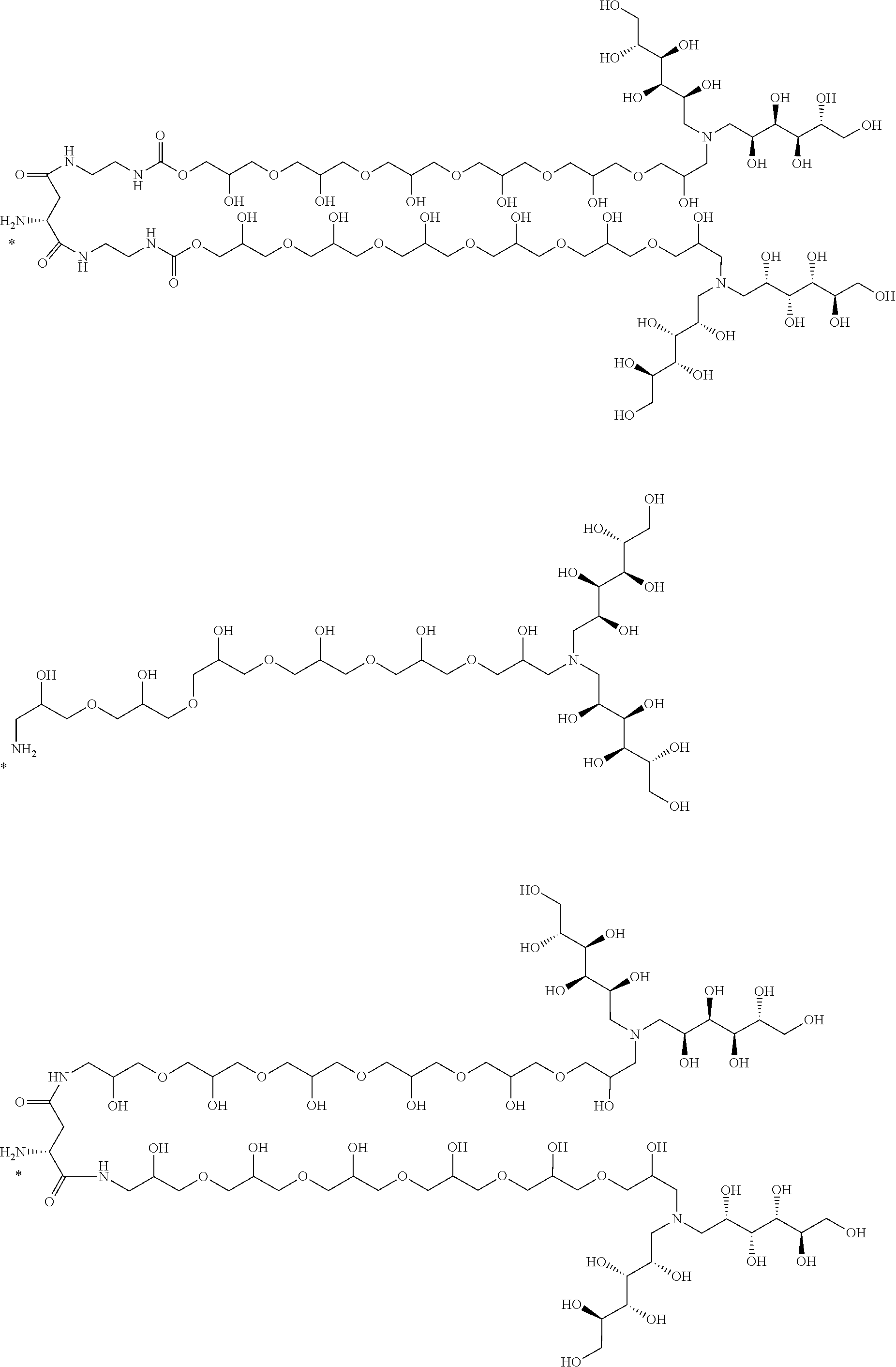

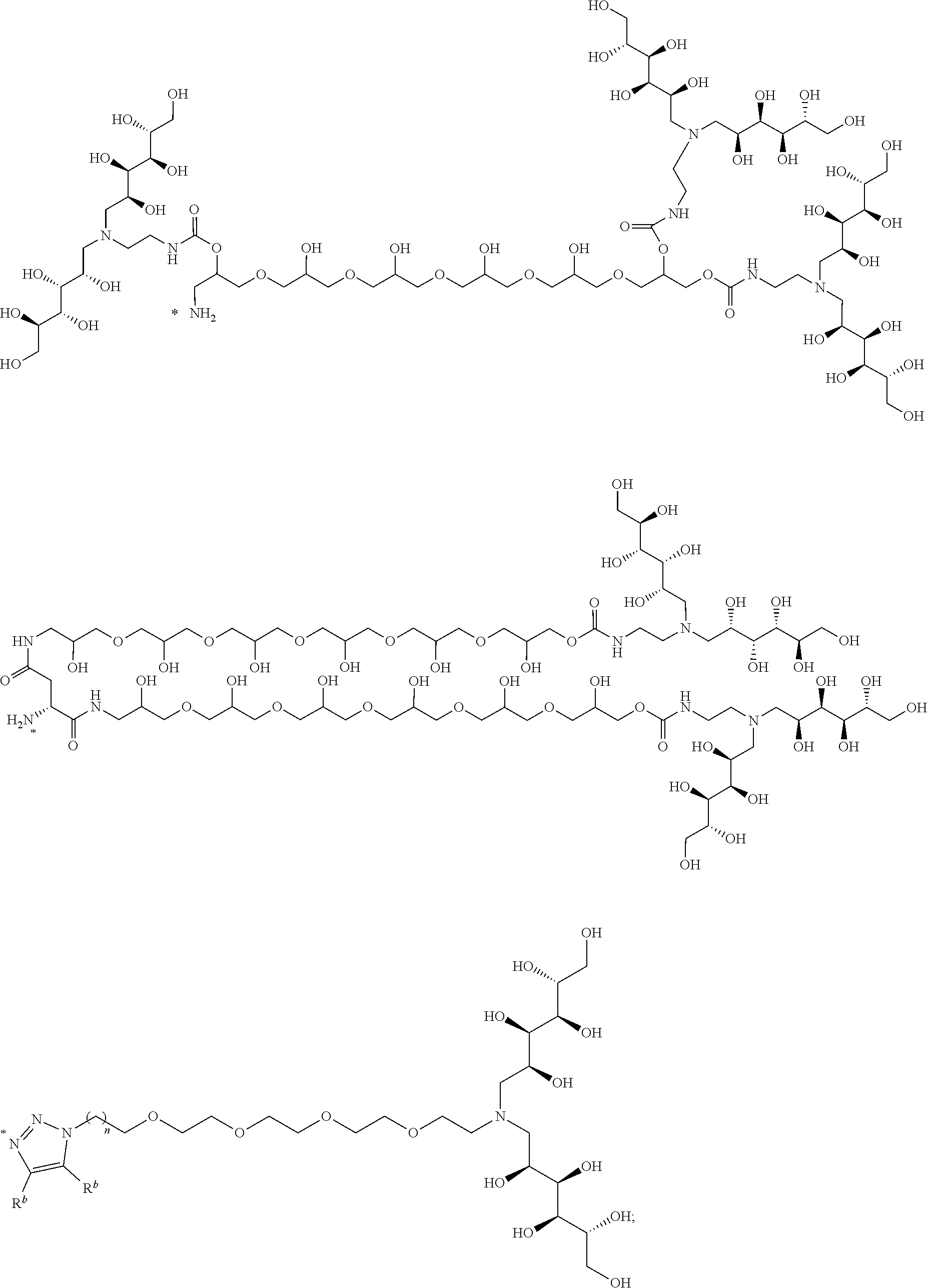

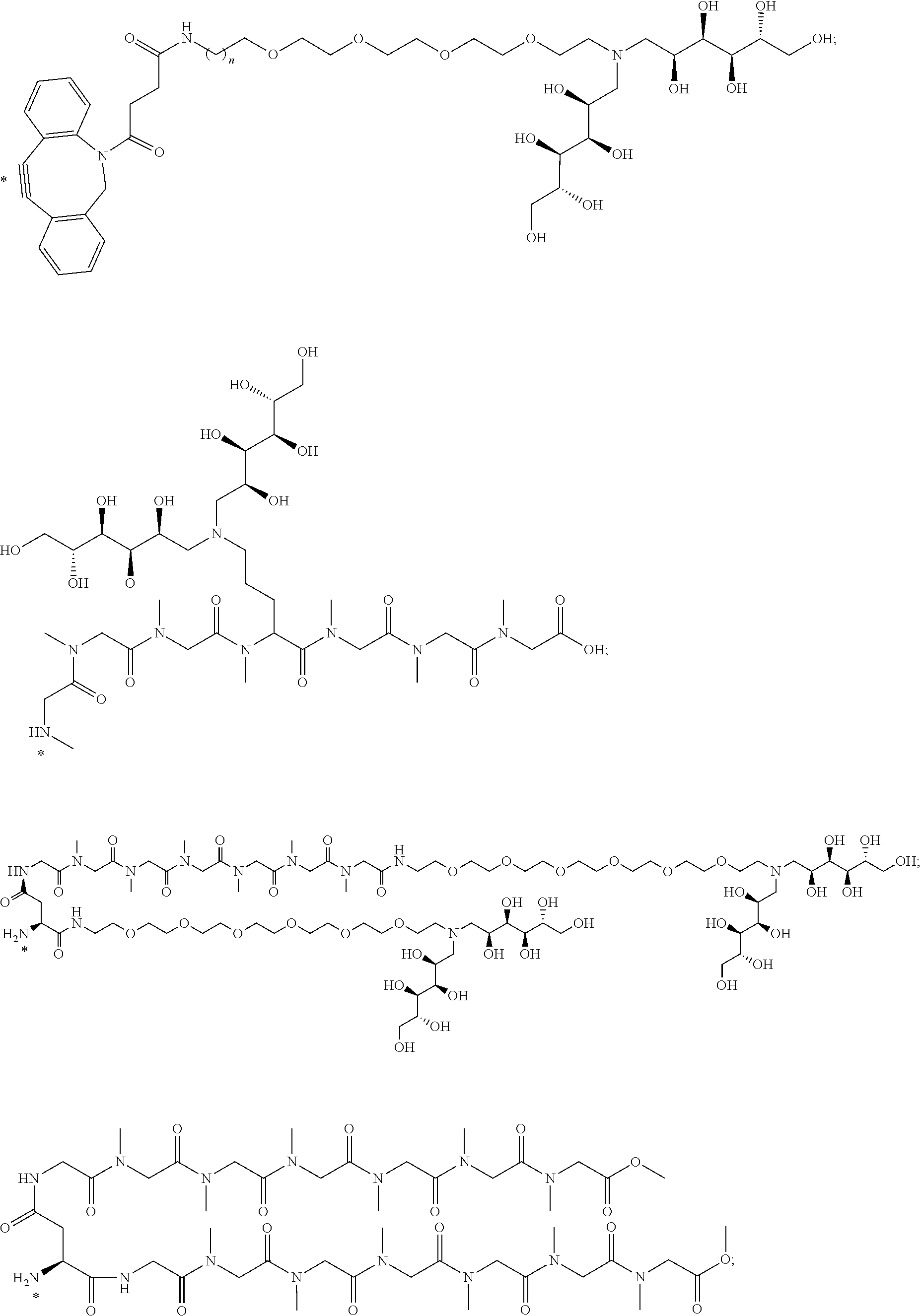

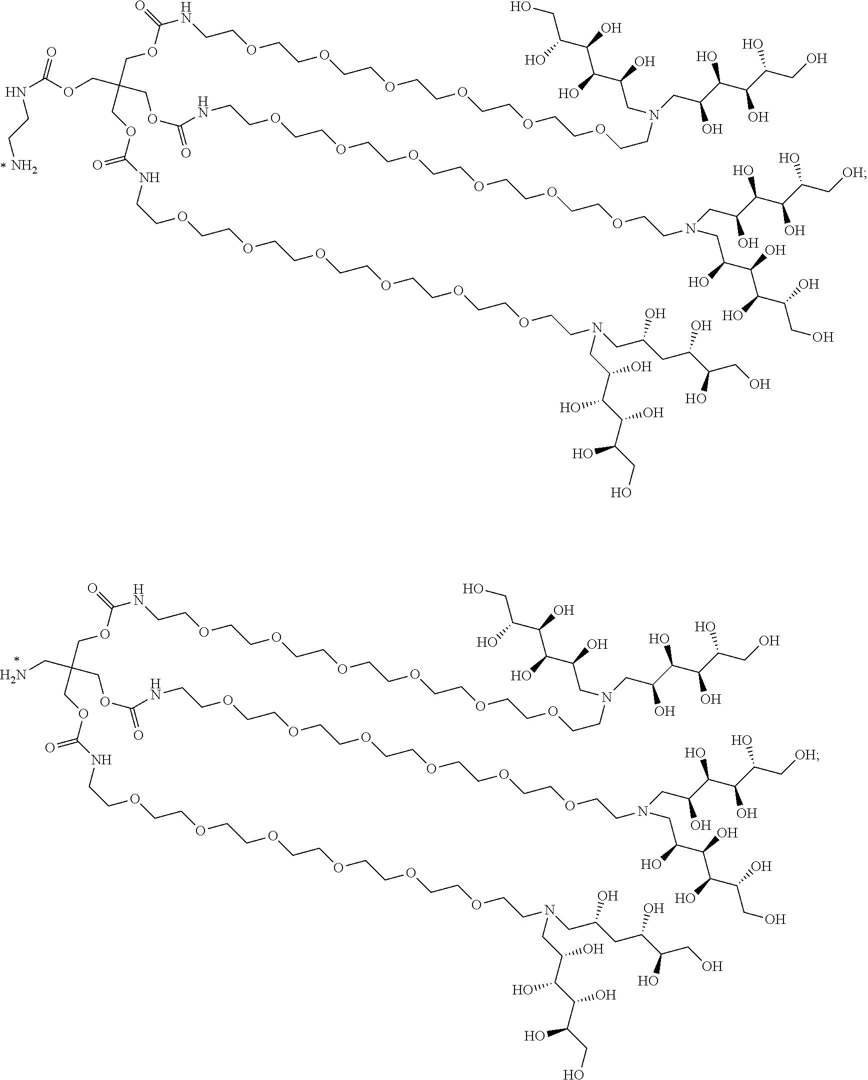

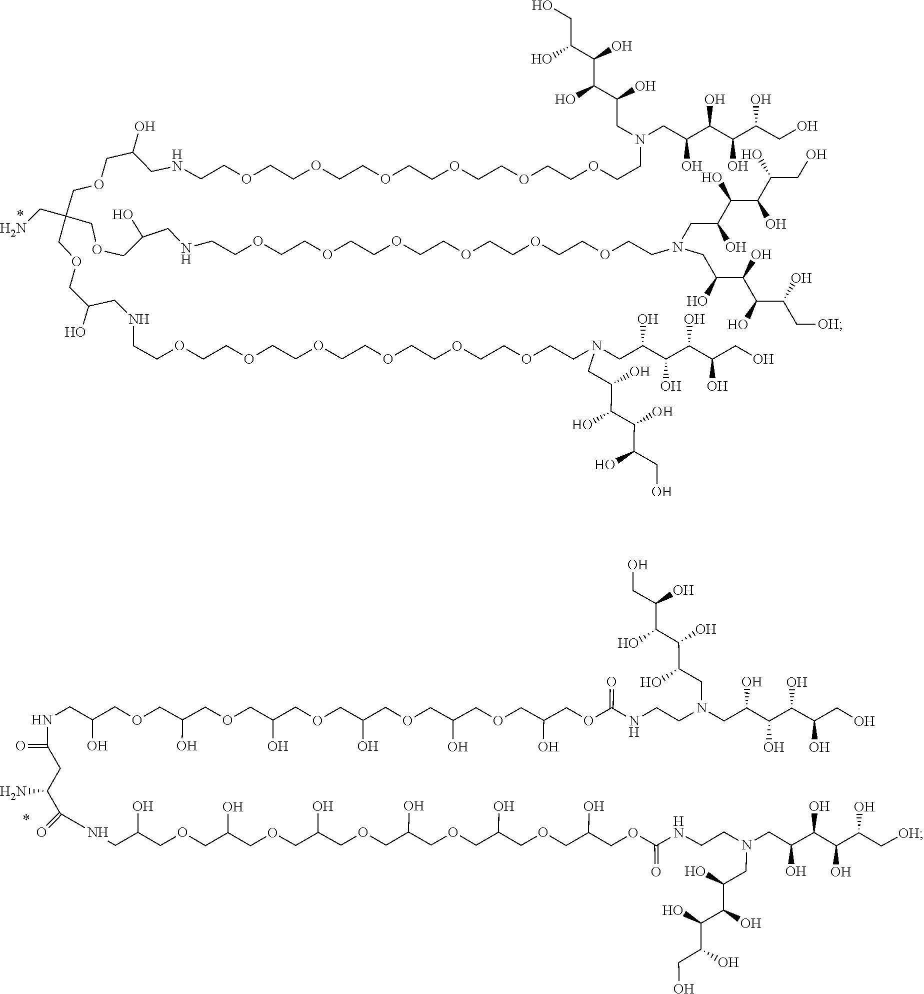

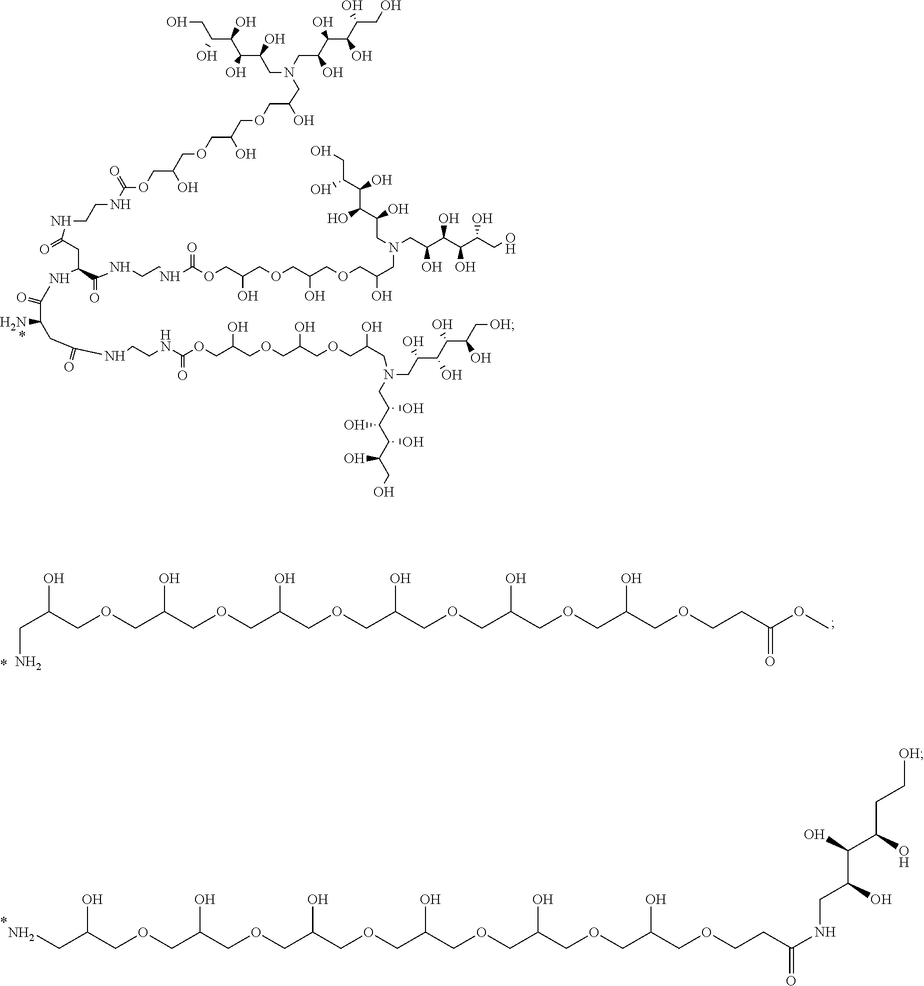

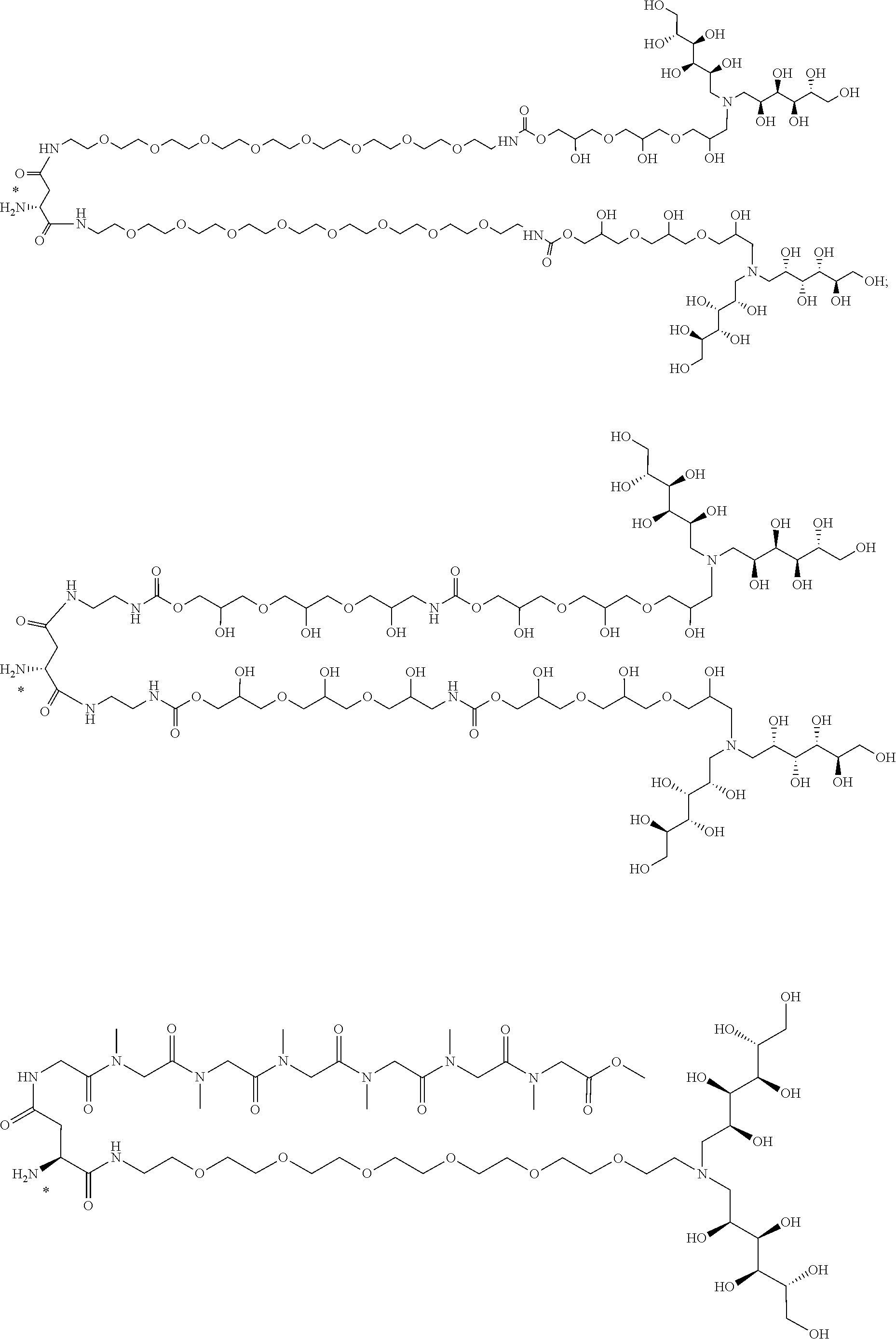

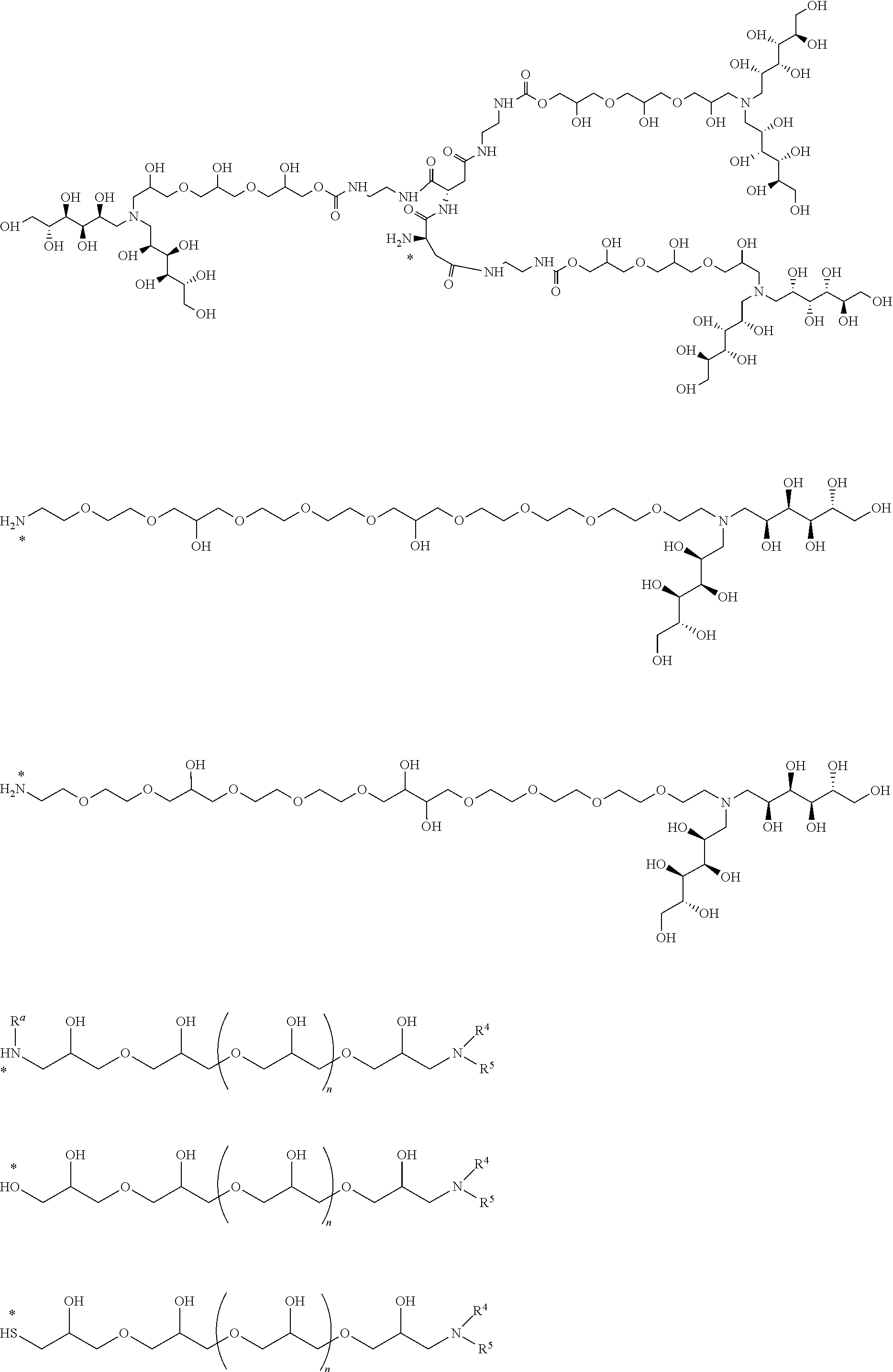

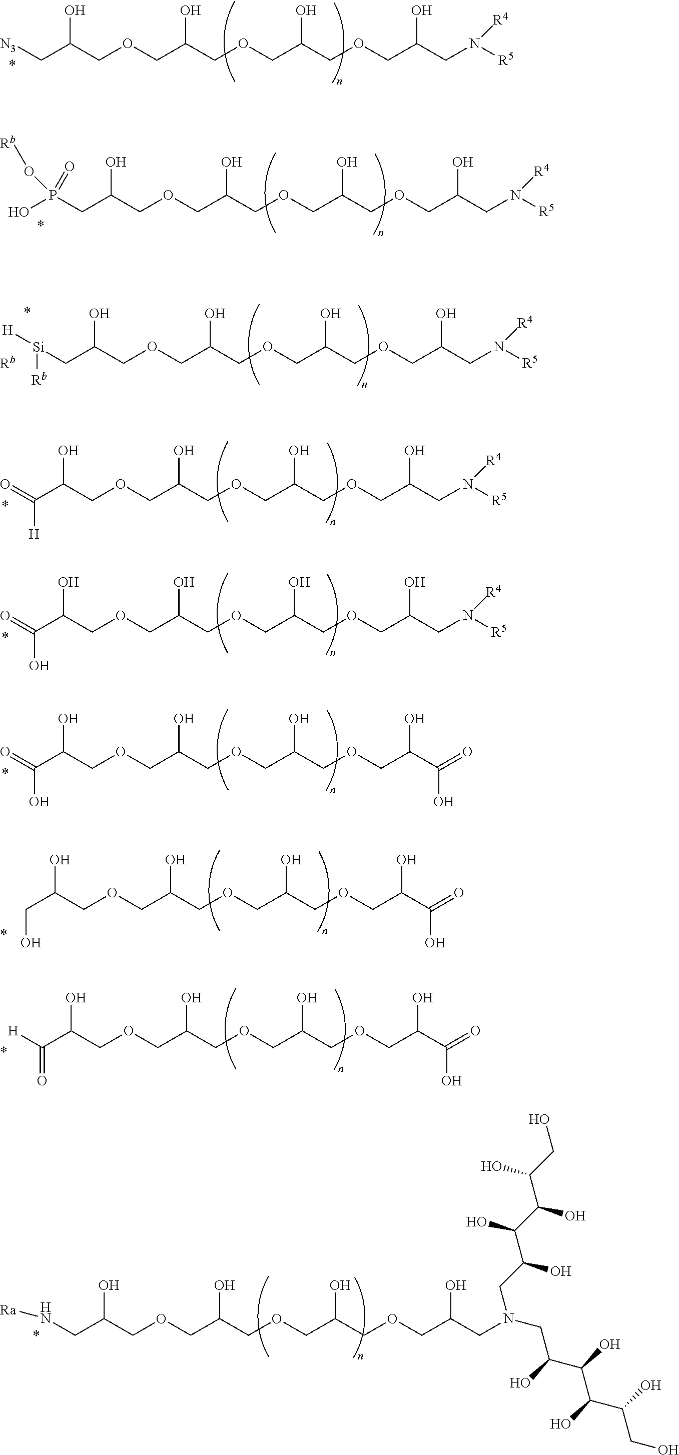

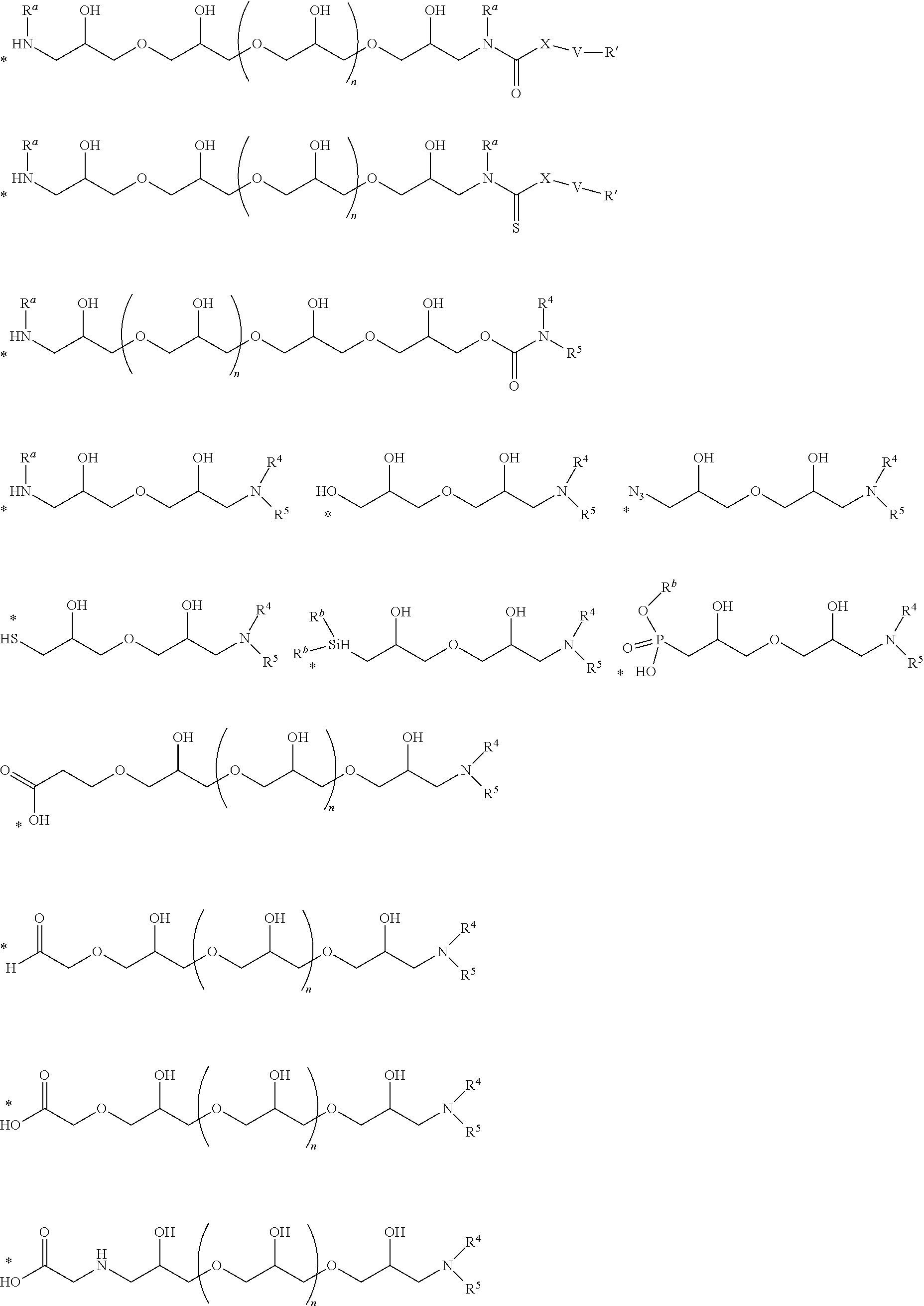

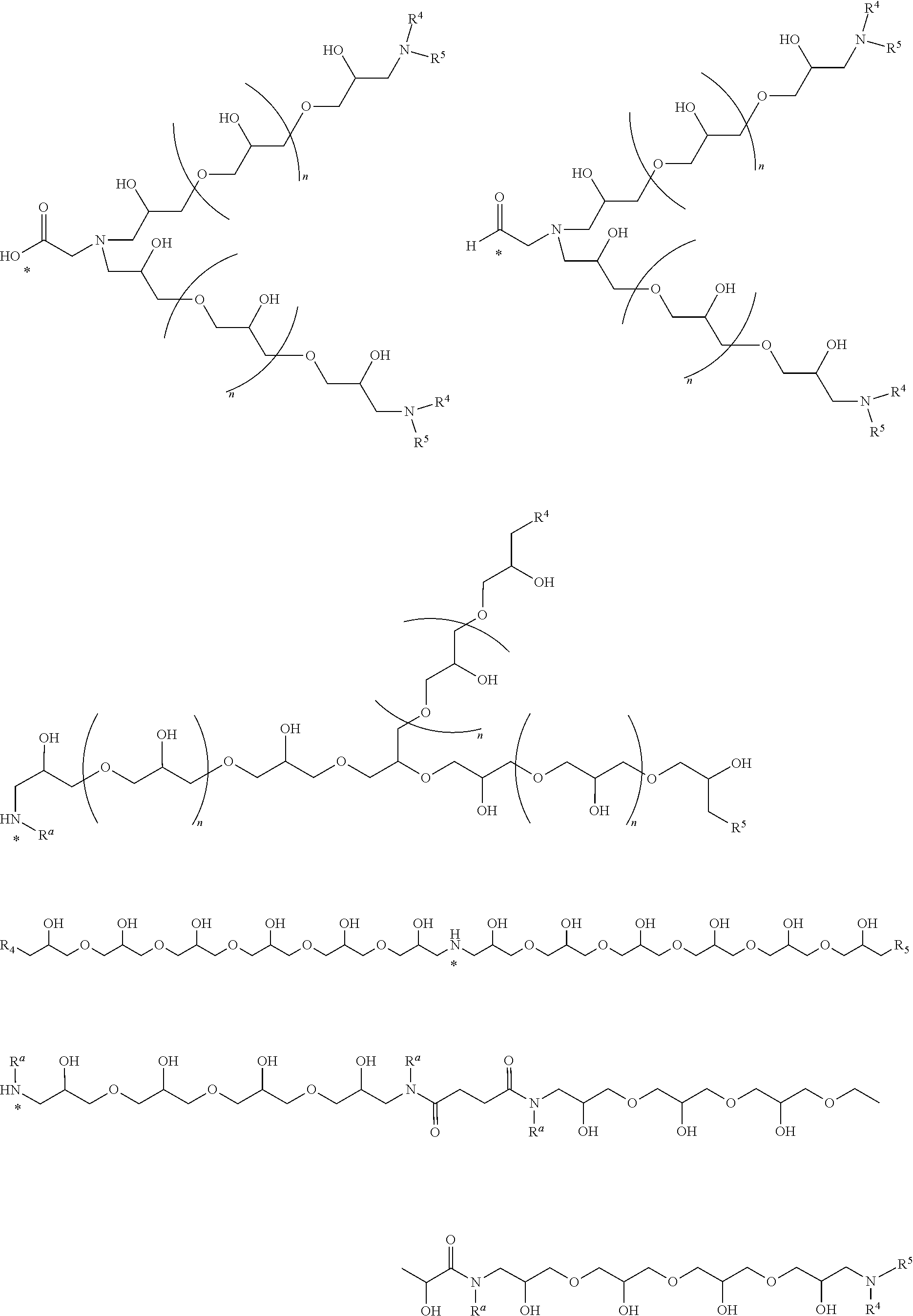

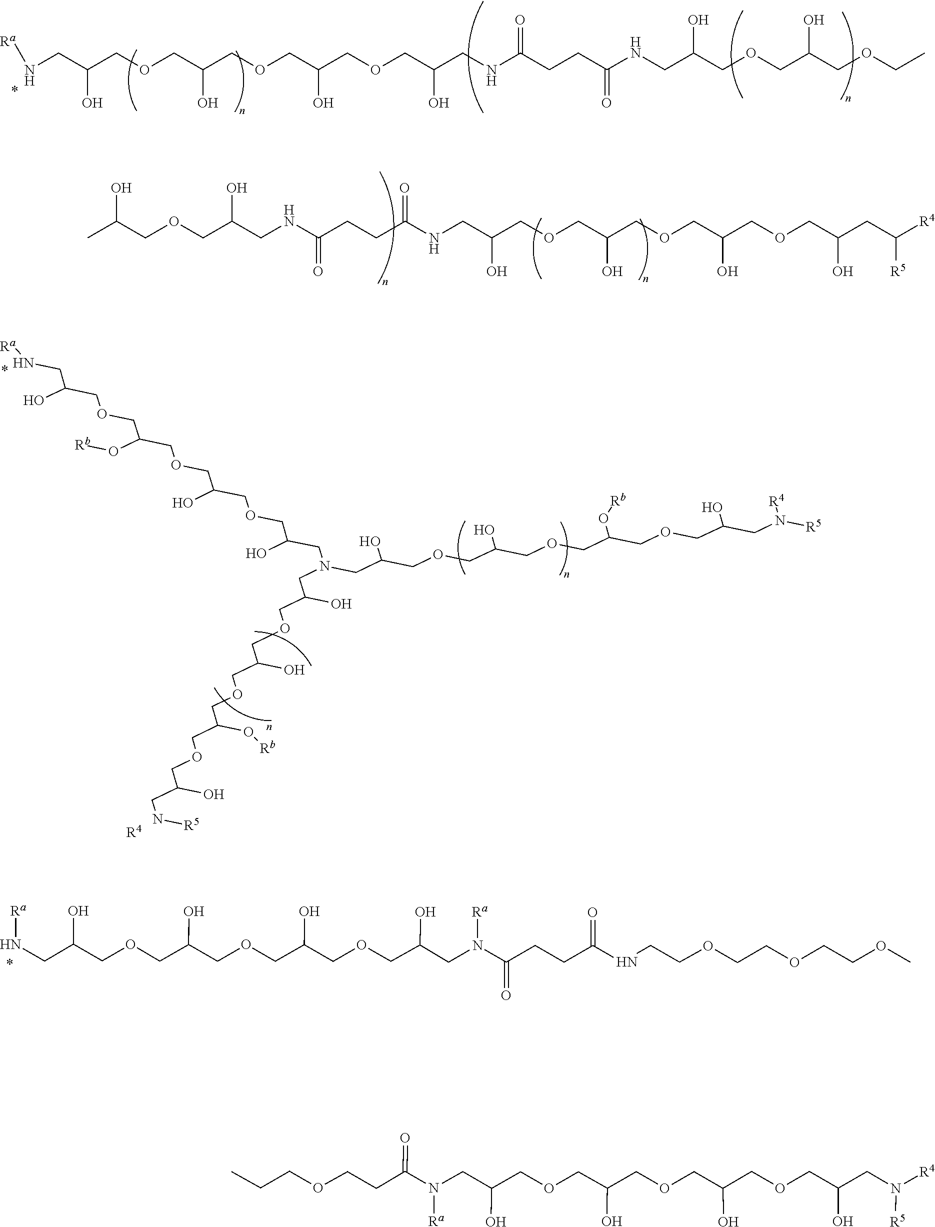

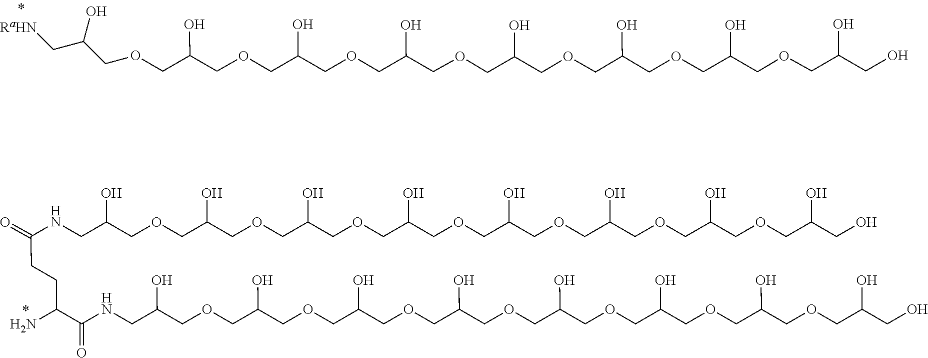

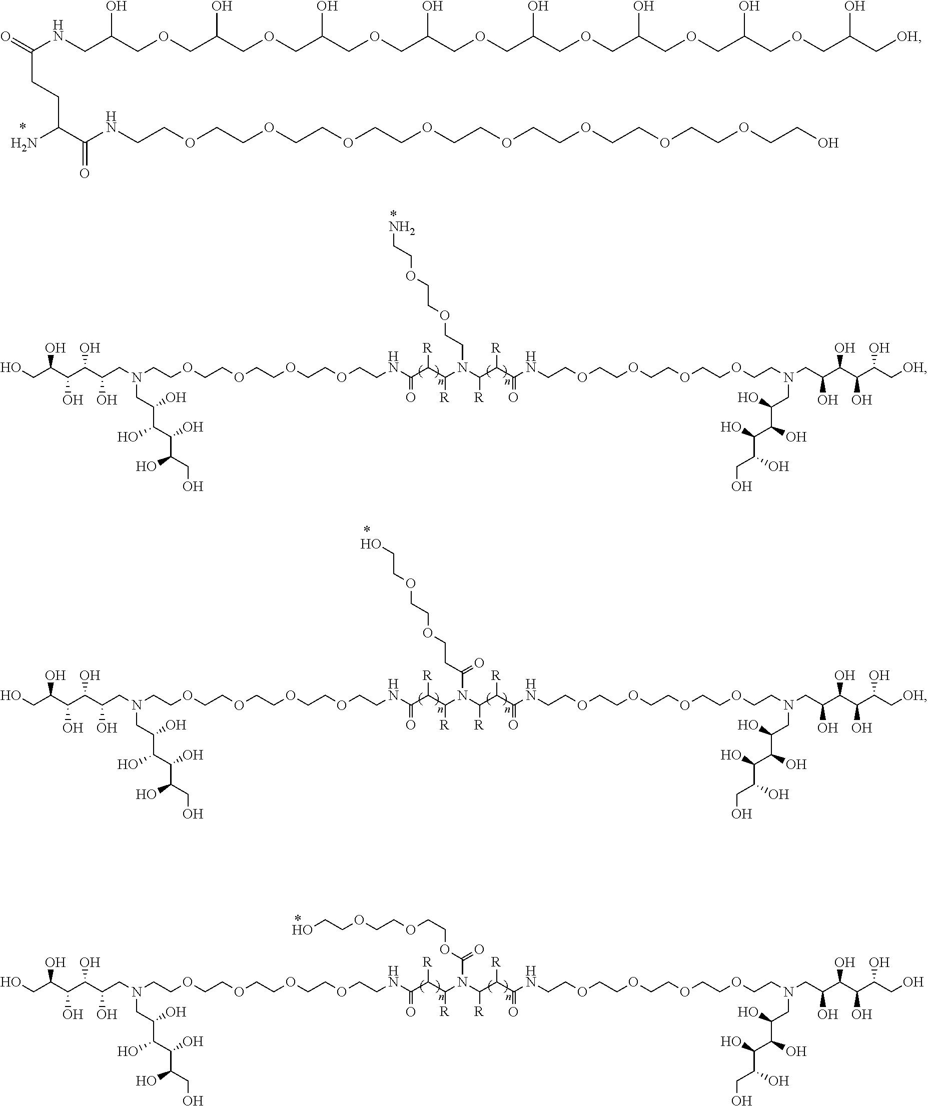

wherein:
(*) indicates the attachment site β or to site R^{b}, or to the enzyme-cleavable group;
each R is independently H or C₁-C₆ alkyl;
R′ is H, C₁-C₆ alkyl, —N(R²⁴)(R²⁵) or —CO₂H;
each n is independently 1 to 12;
X is O, NR or —CH₂—;
V is bond or C₁-C₆ alkyl;
   one of R²⁴ and R²⁵ is selected from a H; polyhydroxyl group; substituted polyhydroxyl group; —C(O)-polyhydroxyl group; substituted —C(O)-polyhydroxyl group; substituted —C(O)—C₁-C₈ alkyl; a chelator; and —C(O)—R²⁸, where R²⁸ is the sugar unit of formula (XII) or (XIII); and the other of R²⁴ and R²⁵ is selected from H; polyhydroxyl group; substituted polyhydroxyl group; —C(O)— polyhydroxyl group; substituted —C(O)-polyhydroxyl group; substituted —C(O)—C₁-C₈ alkyl; a chelator; —C(O)—R²⁸, where R²⁸ is the sugar unit of formula (XII) or (XIII); and polyethylene glycol, optionally having 1 to 24 ethylene glycol subunits, provided that R²⁴ and R²⁵ are not both H.

Embodiment 62. The linker compound of any one of Embodiments 1-14, the polar group has a formula selected from:

(a) ˜R⁴⁰—(R⁴³—R⁴¹—[O—CH₂—CH₂]ₙ₄₀—R⁴⁶—[O—CH₂—CH₂]ₙ₄₀—R⁴²—R⁴³—(NR⁴⁴R⁴⁵)ₙ₄₁)ₙ₄₂ (XLIII)
or a stereoisomer or salt thereof, wherein:
R⁴⁰ is an attachment group to site R^{b}, or to the enzyme-cleavable group;
R⁴¹ and R⁴² are each, independently, a bond or C₁-C₆ alkylene;
each R⁴³ is, independently, selected from a bond, C₁-C₁₂ alkylene, —OC₁-C₁₂ alkylene, —C(═O)—, —NH—C₁-C₁₂ alkylene, —C₁-C₁₂ alkylene-NH—, —C(O)—C₁-C₁₂ alkylene, —C₁-C₁₂ alkylene-C(O)—, —NH—C₁-C₁₂ alkylene-C(O)—, —C(O)—C₁-C₁₂ alkylene-NH—, —NH—C(O)—NH—, —NH—C(O)—, —NH—C(O)—C₁-C₁₂ alkylene, —C(O)—NH—C₁-C₁₂ alkylene, C₁-C₁₂alkylene-NH—C(O)—, -heteroarylene, heteroaryl-C₁-C₁₂ alkylene, heteroaryl-C₁-C₁₂ alkylene-C(O)—, or —C(O)NR⁴⁶R⁴⁷, wherein one of R⁴⁶ and R⁴⁷ is H or C₁-C₁₂ alkylene and the other is C₁-C₁₂ alkylene;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H;
each R⁴⁶ is independently selected from —NR⁵⁰—, —NR⁵⁰—C₁-C₆alkylene-NR⁵⁰—, —NR⁵⁰—C(O)—NR⁵⁰—S(O)₂—NR⁵⁰— or —NR⁵⁰—C(O)—C₁₋₆alkylene-;
each R⁵⁰ is independently selected from H, C₁-C₆ alkyl, or polyhydroxyl group;
each n40 is independently 2 to 26;
n41 is 1 to 6; and
n42 is 1 to 6;

(b) ˜R⁴⁰—(R⁵¹—[O—CH₂—CH₂]ₙ₄₃—R⁵²—X₁—R⁵⁵—X₂—R⁵³—[O—CH₂—CH₂]ₙ₄₃—R⁵⁴—[X₃—R⁵⁶]ₙ₄₄—R⁵⁷)ₙ₄₅ (XLIV)
or a stereoisomer or salt thereof, wherein:
R⁴⁰ is an attachment group to site R^{b}, or to the enzyme-cleavable group;
R⁵¹, R⁵², R⁵³ and R⁵⁴ are each, independently, a bond or C₁-C₆ alkylene;
X₁, X₂ and X₃ are each independently —NR^{N}—C(O)— or —C(O)—NR^{N}—;
each R^{N} independently represent H, C₁-C₆ alkyl, or polyhydroxyl group;
R⁵⁵ and R⁵⁶ each independently represent a bivalent polyhydroxyl group;
R⁵⁷ is H, OH or C₁-C₆ alkyl;
each n43 is independently 0 to 26, with the proviso that at least one n43 is 1 to 26;
n44 is 0 to 10; and
n45 is 1 or 2; or

(c) ˜R⁴⁰—R⁵¹—[O—CH₂—CH₂]ₙ₄₃—R⁵²—N—(R⁵³—X₁—R⁵⁴—[O—CH₂—CH₂]ₙ₄₃—(NR⁴⁴R⁴⁵))₂ (XLV)

or a stereoisomer or salt thereof, wherein:
R⁴⁰ is an attachment group to site R^{b}, or to the enzyme-cleavable group;
R⁵¹, R⁵³ and R⁵⁴ are each, independently, a bond or optionally-substituted C₁-C₆ alkylene;
R⁵² is a bond, C₁-C₆ alkylene, —C(O)— or —O—C(O)—;
each X₁ is independently —NR^{N}—C(O)— or —C(O)—NR^{N}—;
each R^{N} independently represent H, C₁-C₆ alkyl, or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n43 is independently 2 to 26.

Embodiment 63. The linker compound of Embodiment 52, the polar group has one of the following structures prior to attachment to the enzyme-cleavable group and/or to the linker unit:

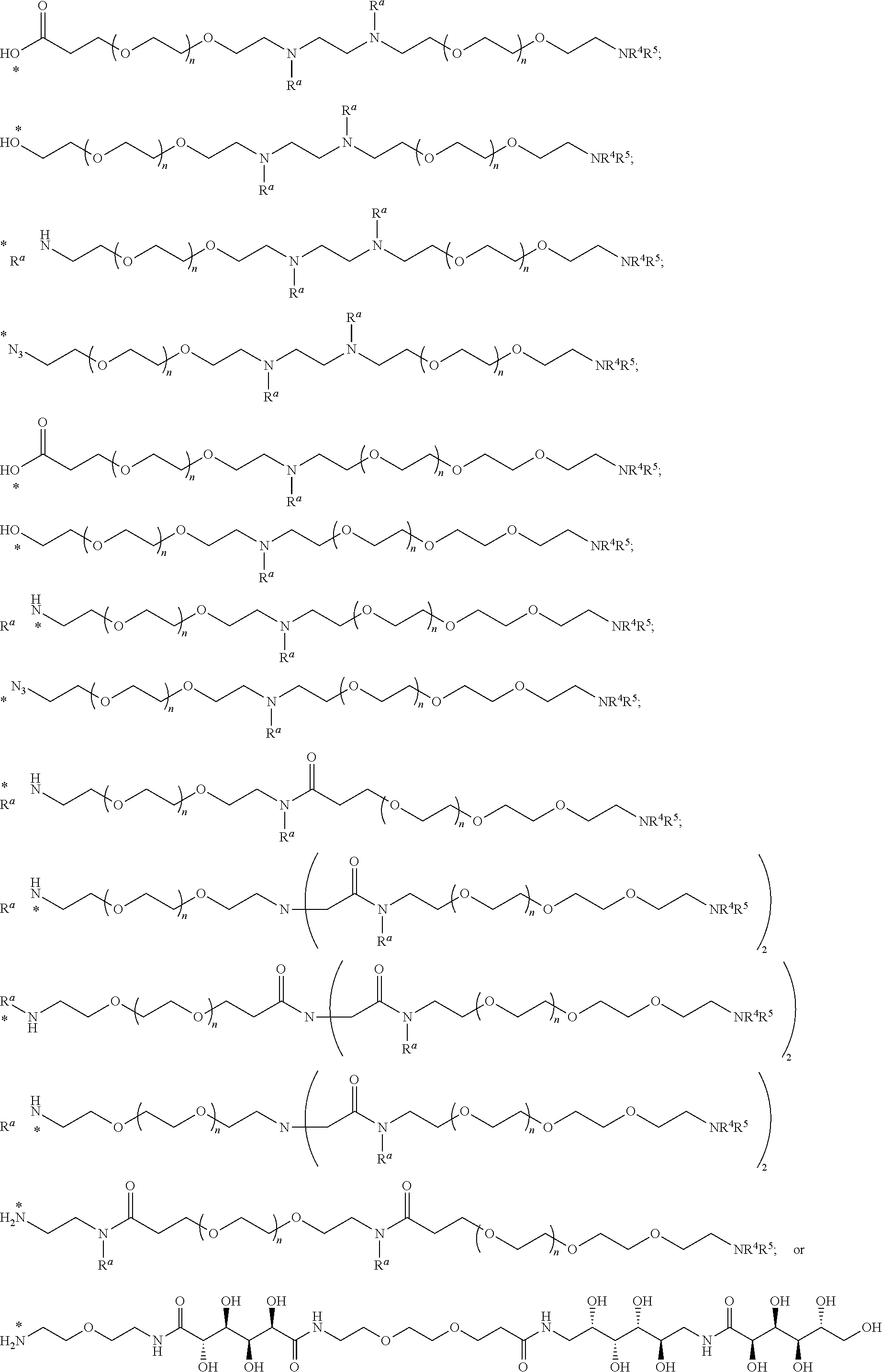

wherein:
(*) indicates the attachment site to site R^{b}, or to the enzyme-cleavable group;
each R is independently H, alkyl or polyhydroxyl group;
R⁴⁴ and R⁴⁵ are each, independently, H, polyhydroxyl group, substituted polyhydroxyl group, —C(O)-polyhydroxyl group, or substituted —C(O)-polyhydroxyl group, wherein optional substituents are selected from sulfate, phosphate, alkyl sulfate, and alkyl phosphate, provided that R⁴⁴ and R⁴⁵ are not both H; and
each n is independently 1 to 12.

Embodiment 64. The linker compound of any one of Embodiments 1-14, the polar group has a formula selected from:

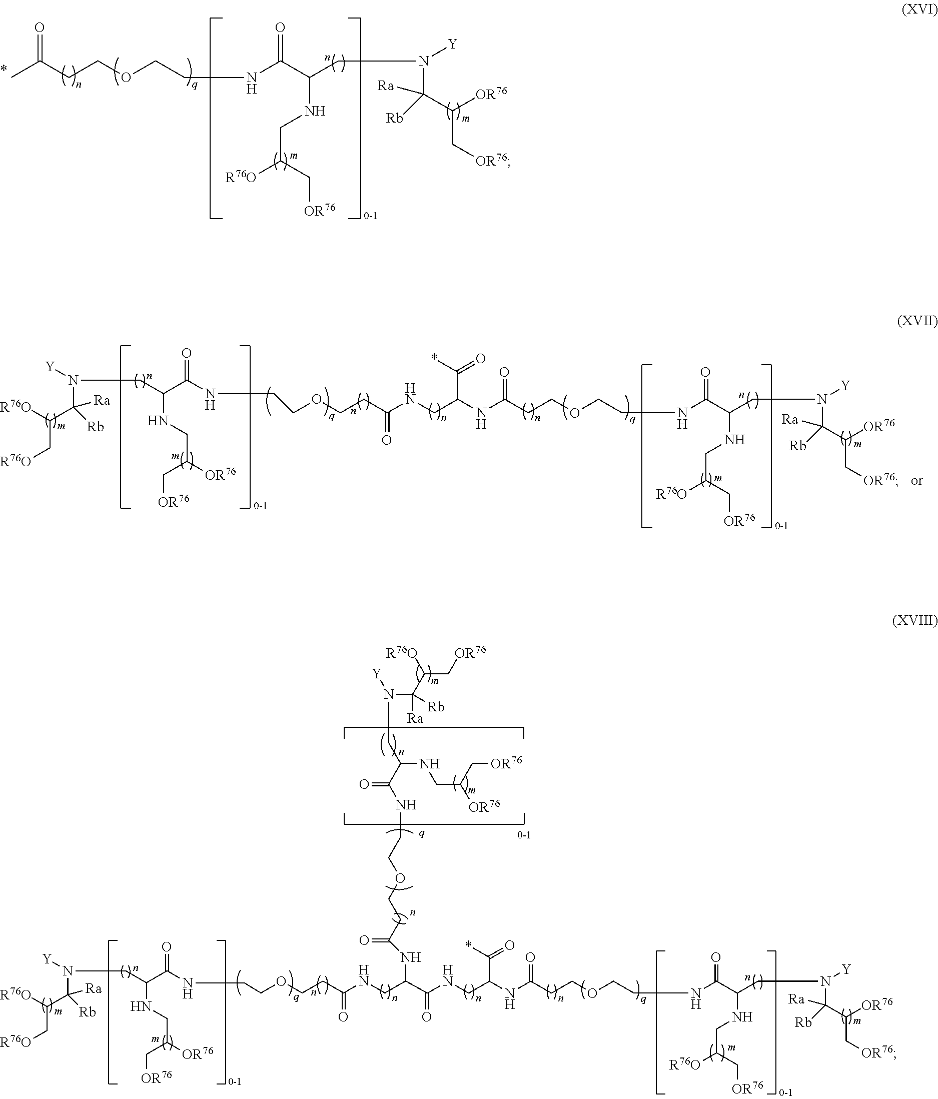

or a stereoisomer or salt thereof, wherein:
each Y is independently R⁷⁶ or

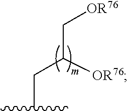

each R⁷⁶ is independently H, acetyl, —P(═O)(OH)₂, or —(CH₂)ᵥ—O—S(═O)₂(OH);
each Rₐ and R_{b} is independently H or Rₐ and R_{b} are taken together with the carbon to which they are attached to form an oxo group;
each q is independently 2-26;
each m is independently 1 to 4;
each n is independently 1 to 4;
each v is independently 1 to 6; and
   each * is an attachment site to R^{b}, or to the enzyme-cleavable group.

Embodiment 65. The linker compound of any one of Embodiments 1-14, the polar group has a formula selected from:

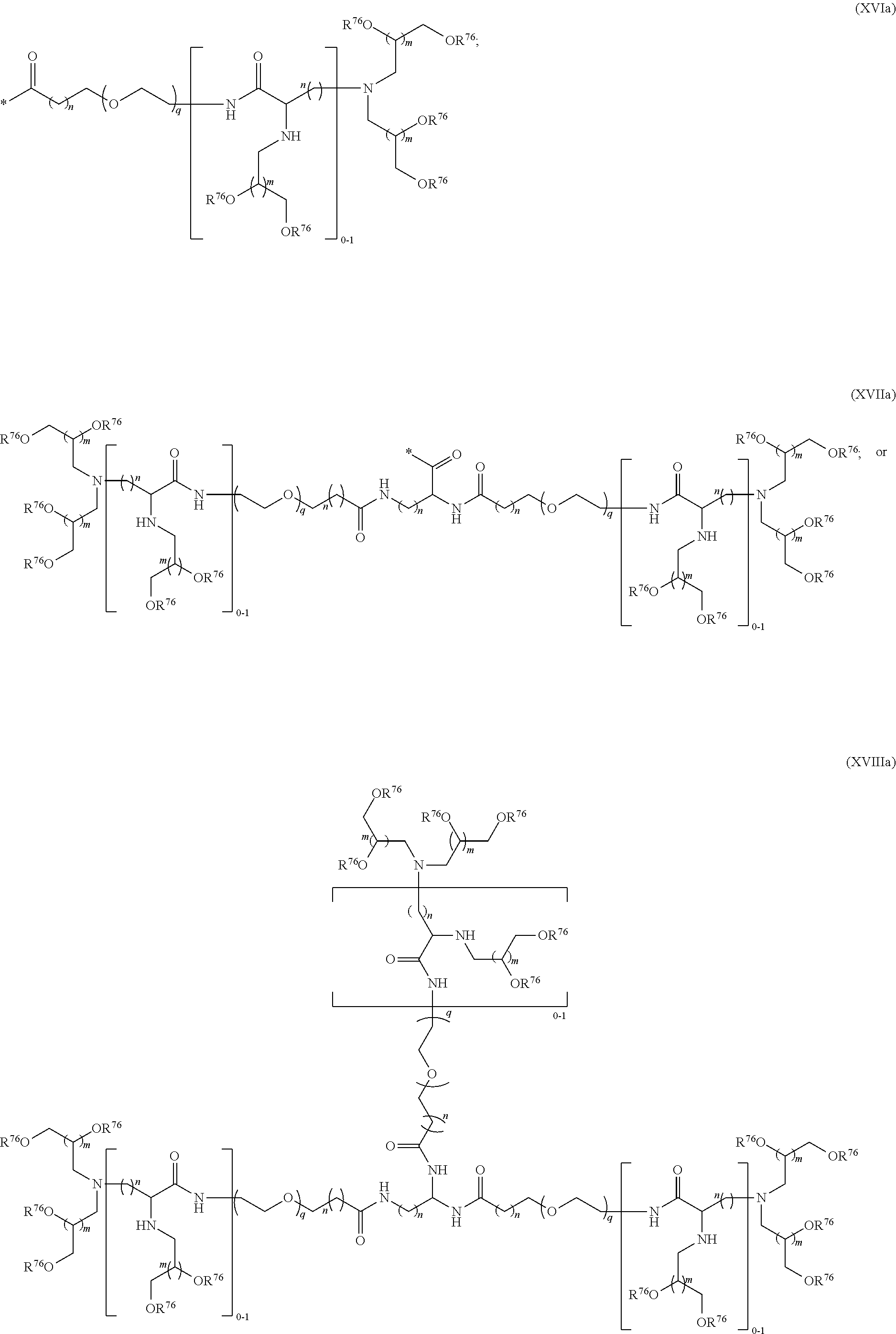

or a stereoisomer or salt thereof, wherein:
each R⁷⁶ is independently H, acetyl, —P(═O)(OH)₂, or —(CH₂)ᵥS(═O)₂(OH);
each q is independently 2-26;
each m is independently 1 to 4;
each n is independently 1 to 4;
each v is independently 1 to 6; and
   each * is an attachment site to R^{b}, or to the enzyme-cleavable group

Embodiment 66. The linker compound of any one of Embodiments 1-14, the polar group has a formula selected from:

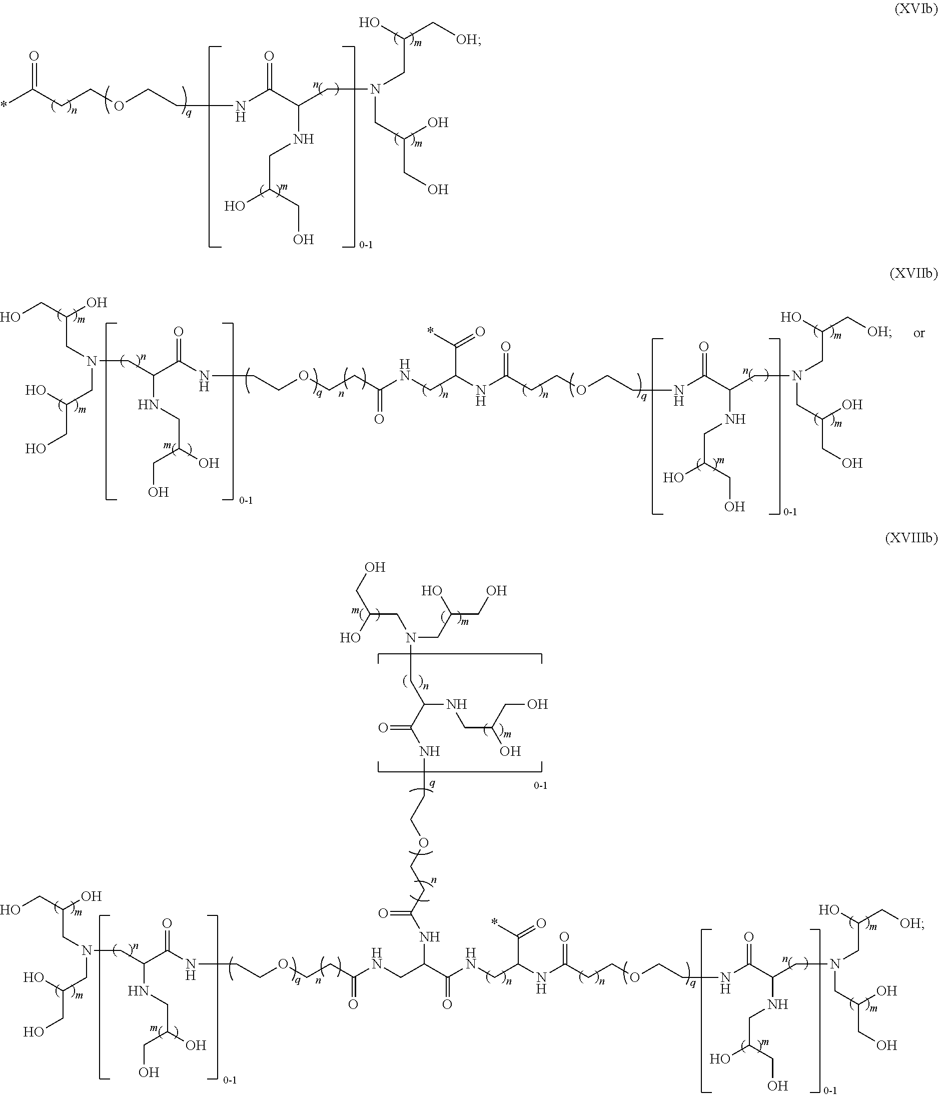

or a stereoisomer or salt thereof, wherein:
each q is independently 2-26;
each m is independently 1 to 4;
each n is independently 1 to 4; and
   each * is an attachment site to R^{b}, or to the enzyme-cleavable group.

Embodiment 67. The linker compound of Embodiment 66, wherein Y is R⁷⁶.

Embodiment 68. The linker compound of Embodiment 66, wherein Y is

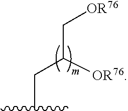

Embodiment 69. The linker compound of Embodiment 66, wherein each R^{a} and R^{b} is independently H.

Embodiment 70. The linker compound of Embodiment 66, wherein R^{a} and R^{b} are taken together with the carbon to which they are attached to form an oxo group.

Embodiment 71. The linker compound of any one of Embodiments 64-66, wherein q is 10-20.

Embodiment 72. The linker compound of any one of Embodiments 64-66, wherein q is 12.

Embodiment 73. The linker compound of any one of Embodiments 1-72, wherein the polar group has one of the following structures prior to attachment to the amino acid unit:

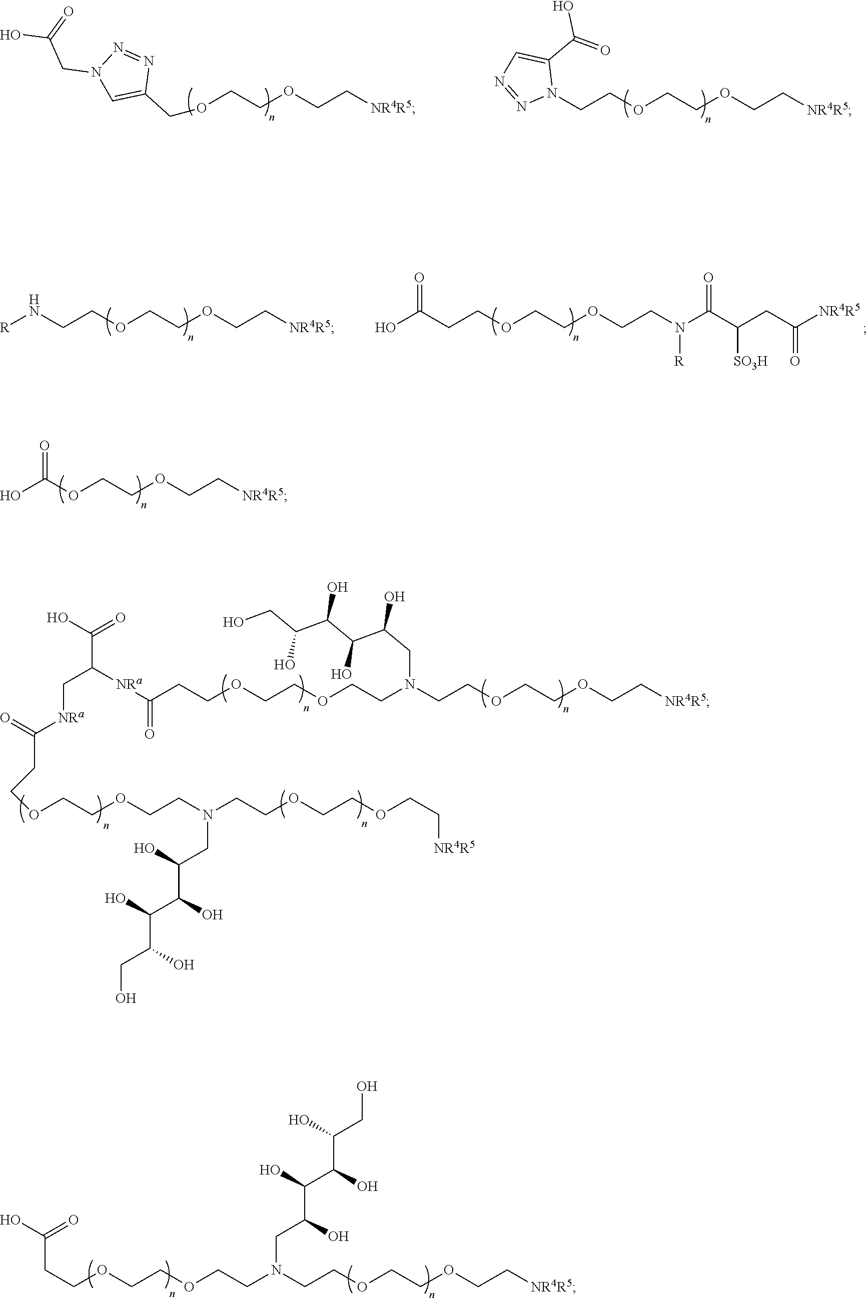

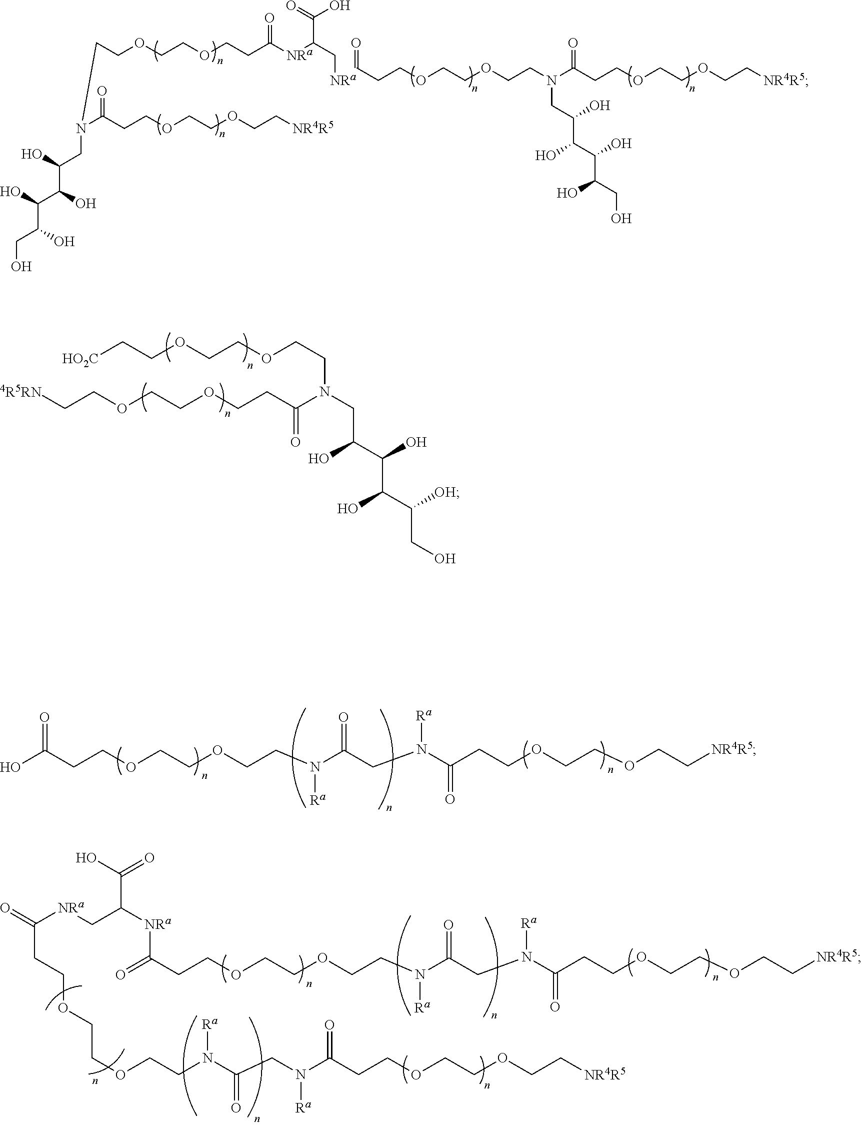

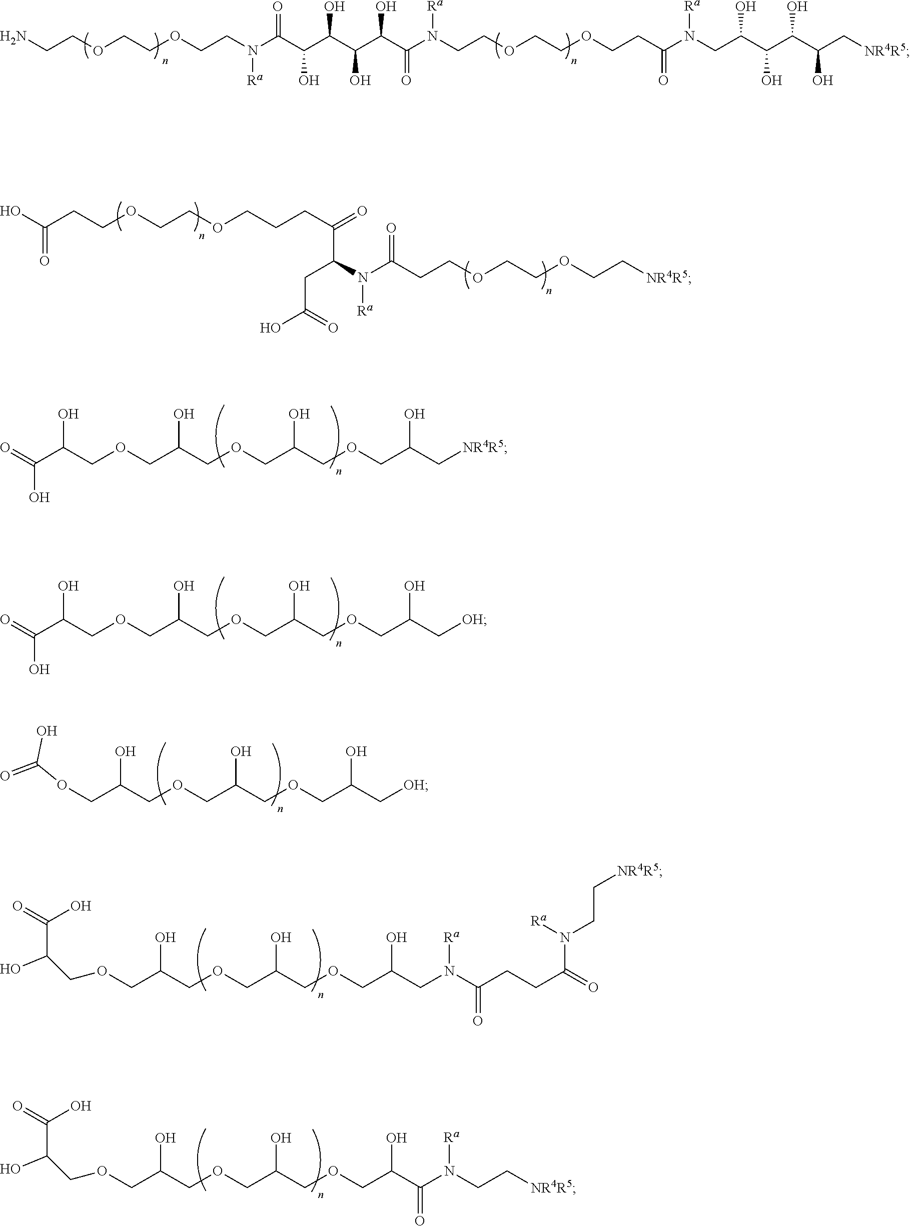

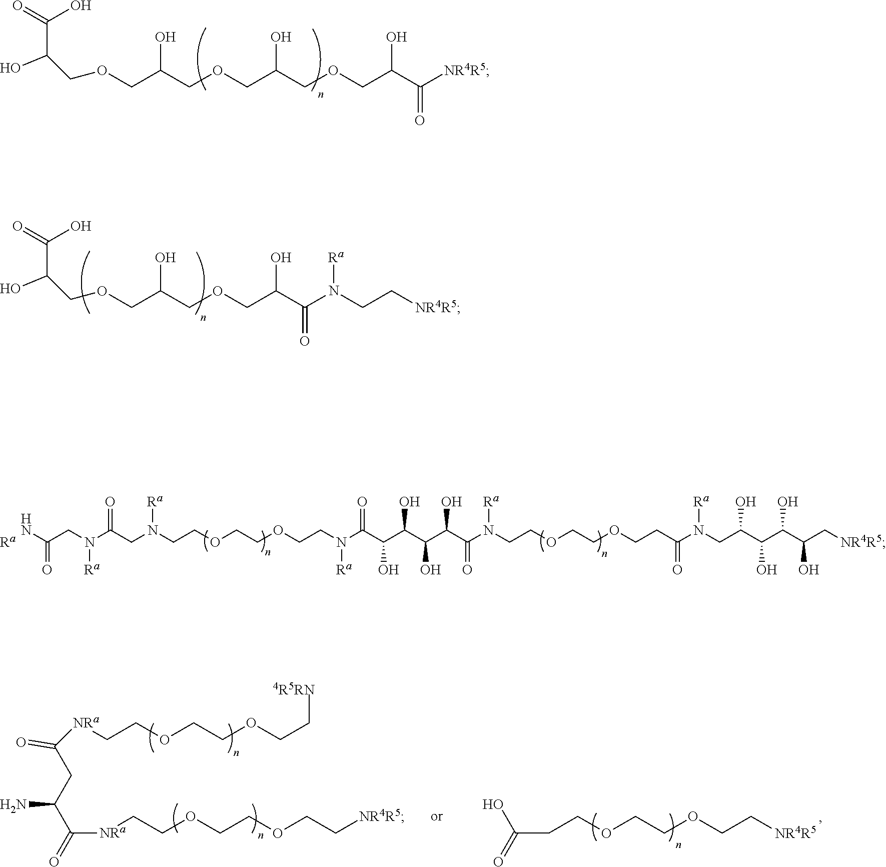

or a stereoisomer thereof, wherein R^{a} is H or C₁₋₆alkyl and n is 1-20.

Embodiment 74. The linker compound of any one of Embodiments 1-73, wherein the polar group has one of the following structures prior to attachment to the amino acid unit:

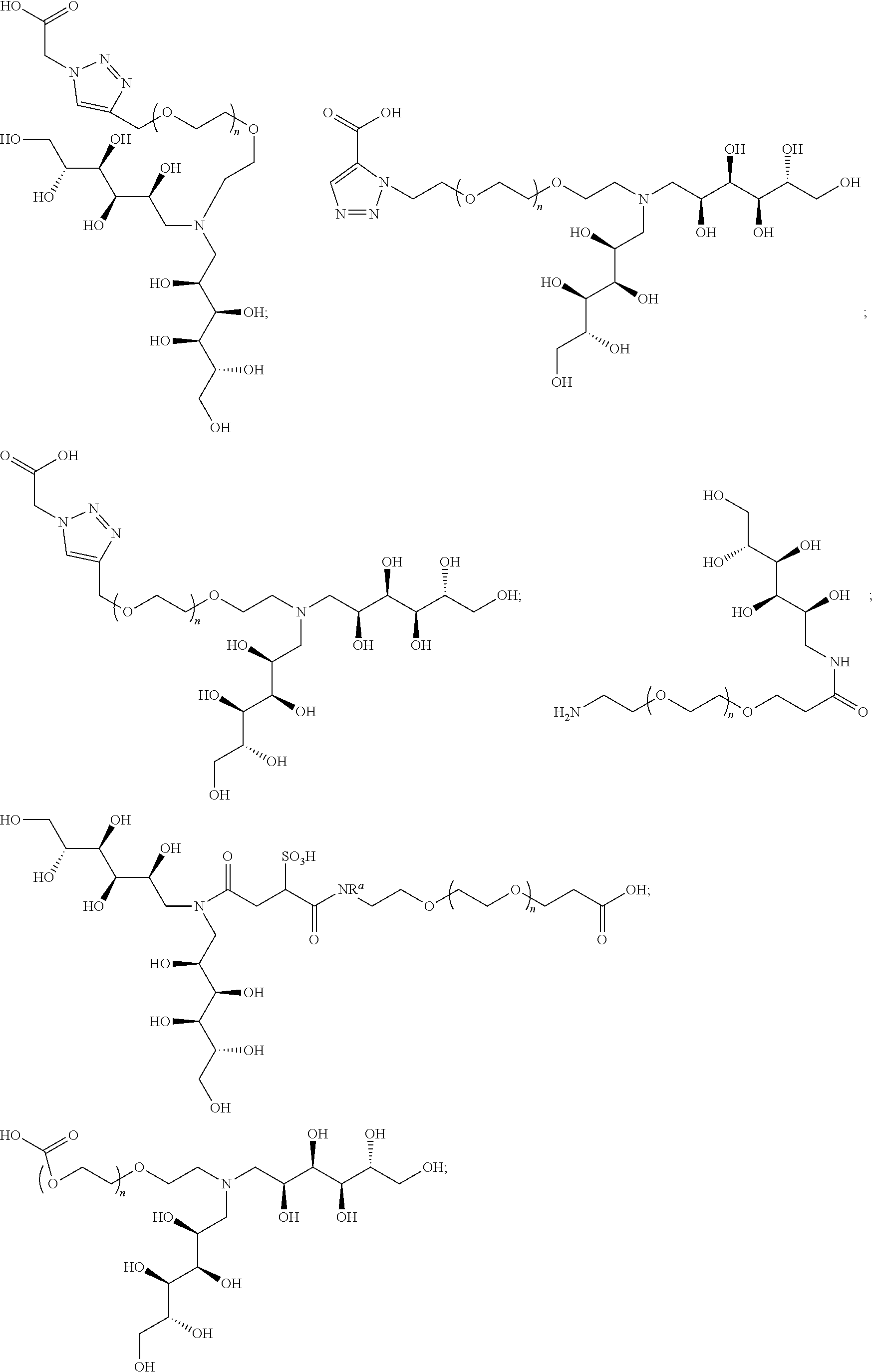

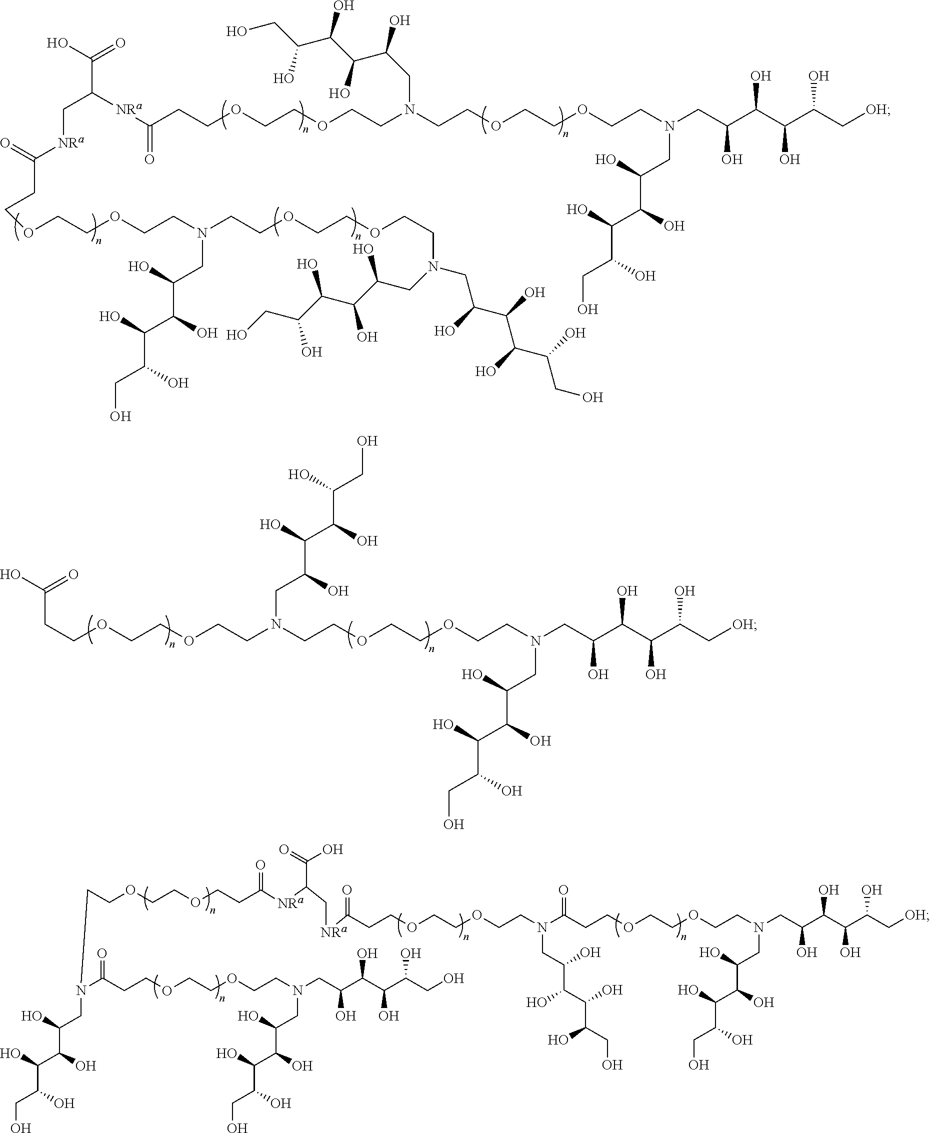

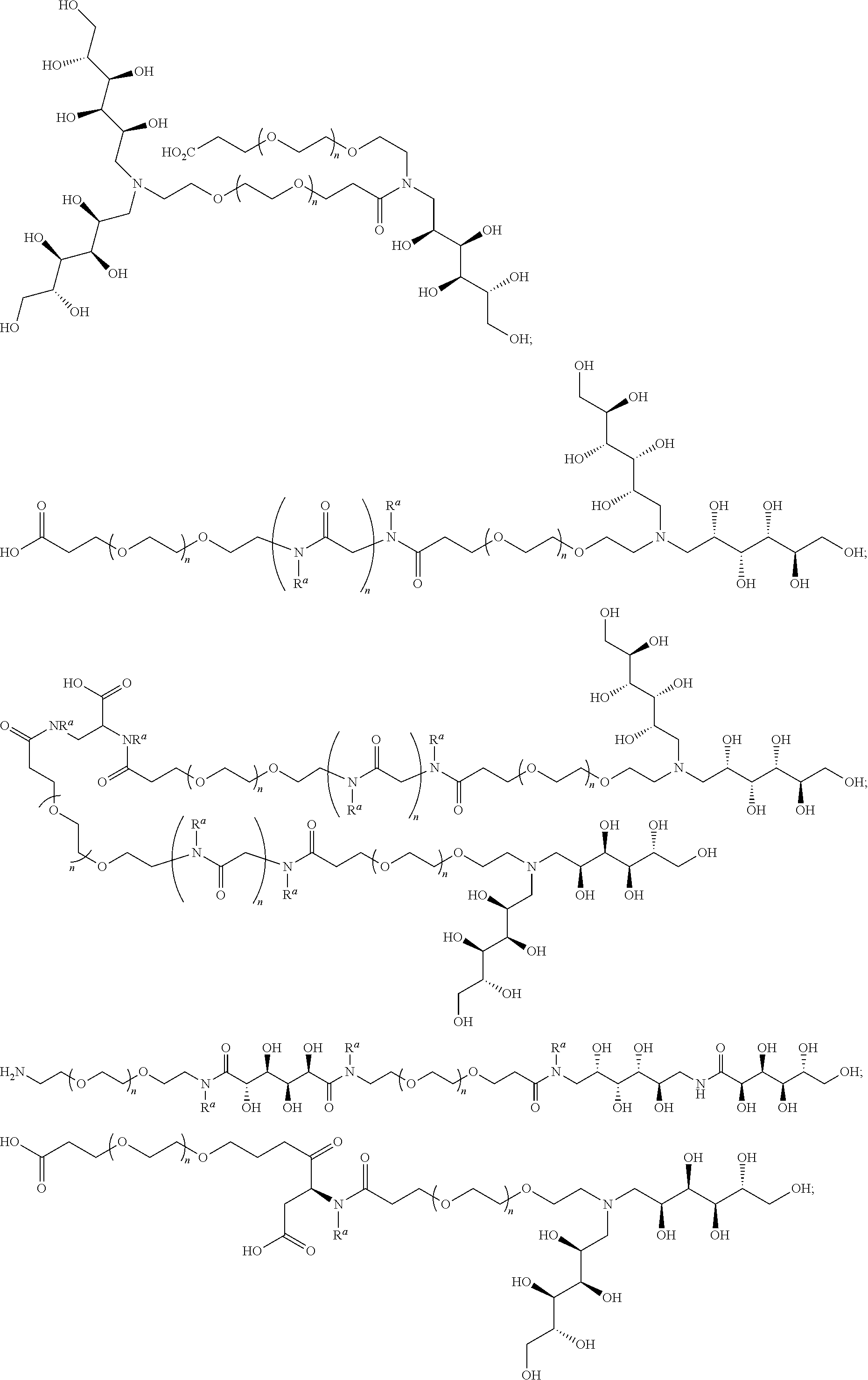

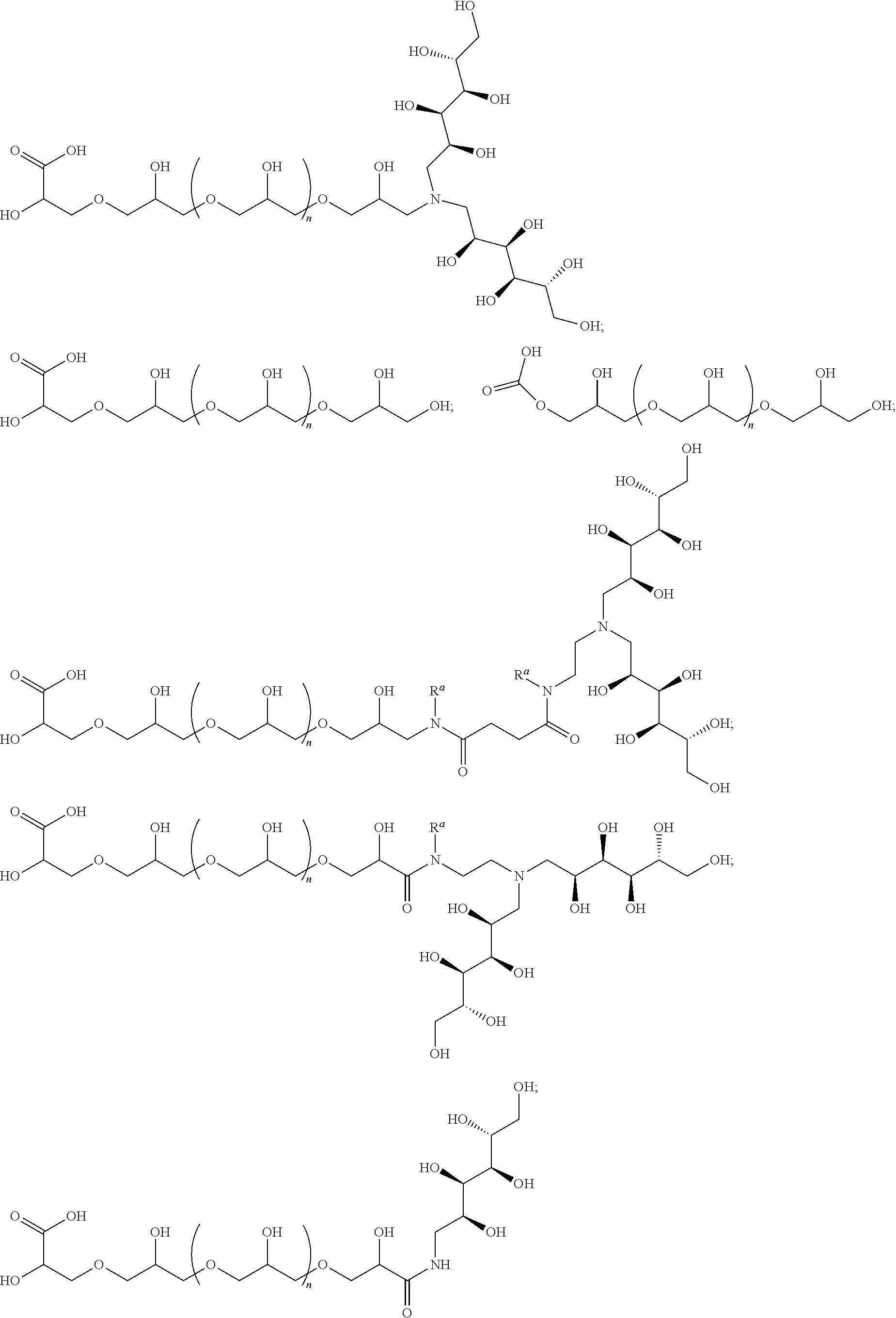

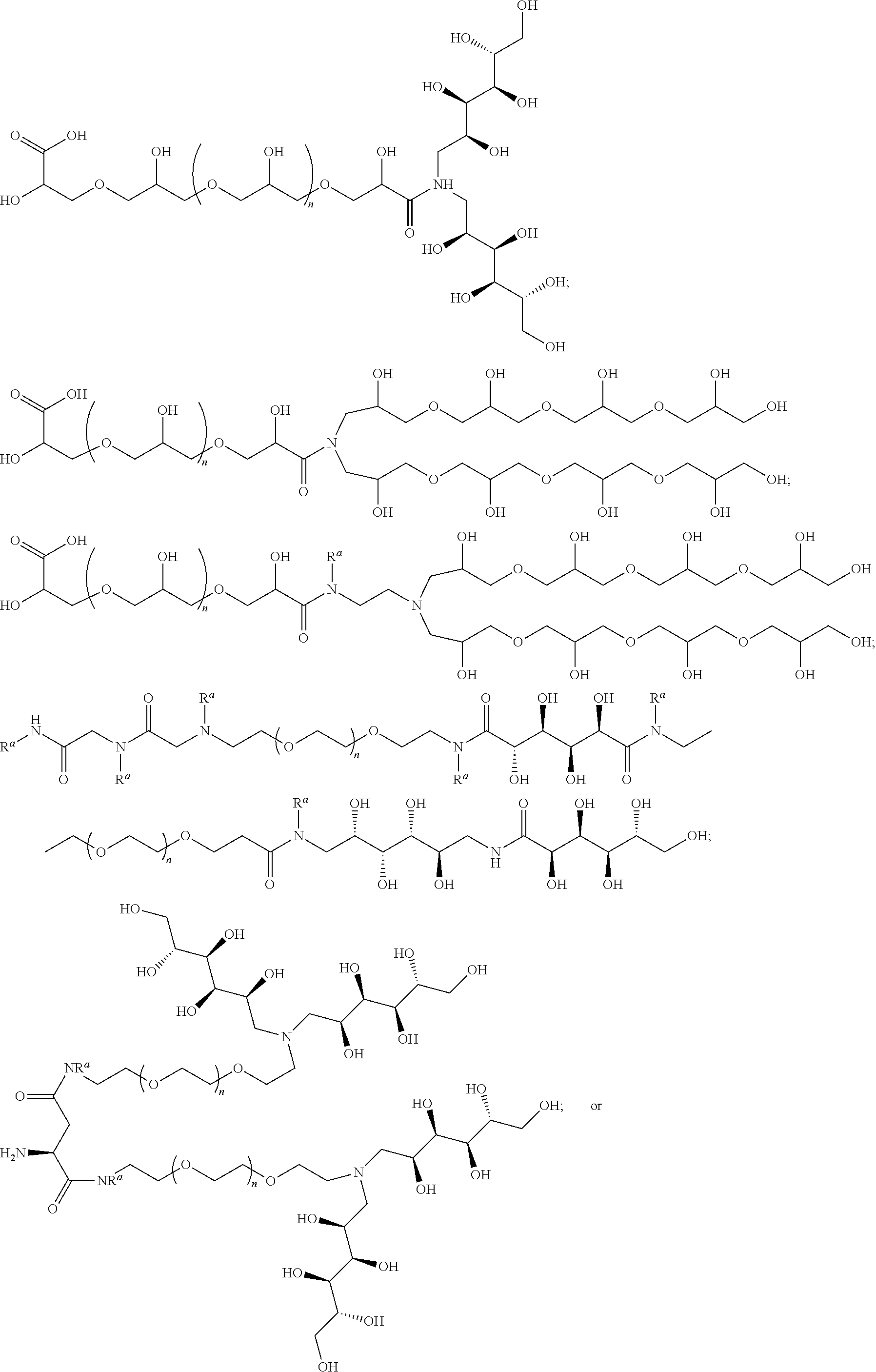

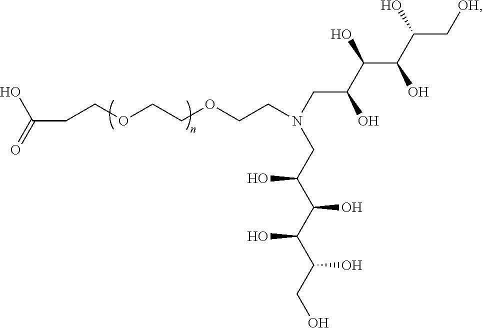

or a stereoisomer thereof, wherein R^{a} is H or C₁₋₆alkyl and n is 1-20.

Embodiment 75. The linker compound of any one of Embodiments 1-74, wherein the polar group has one of the following structures prior to attachment to the amino acid unit:

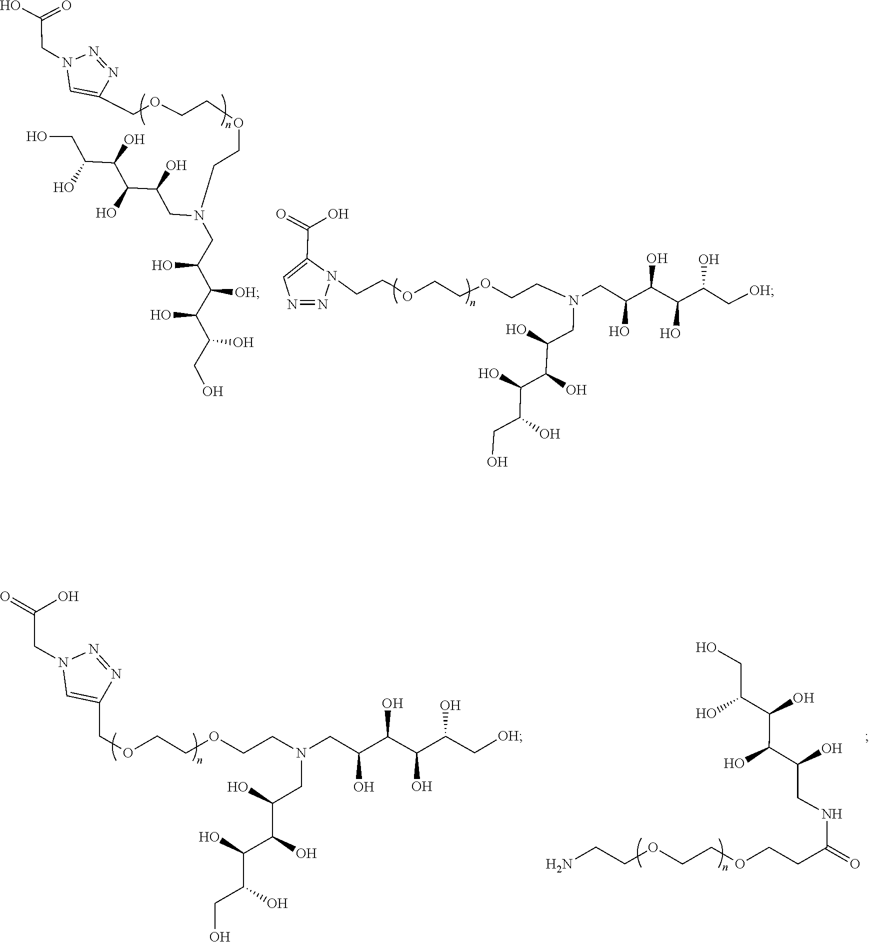

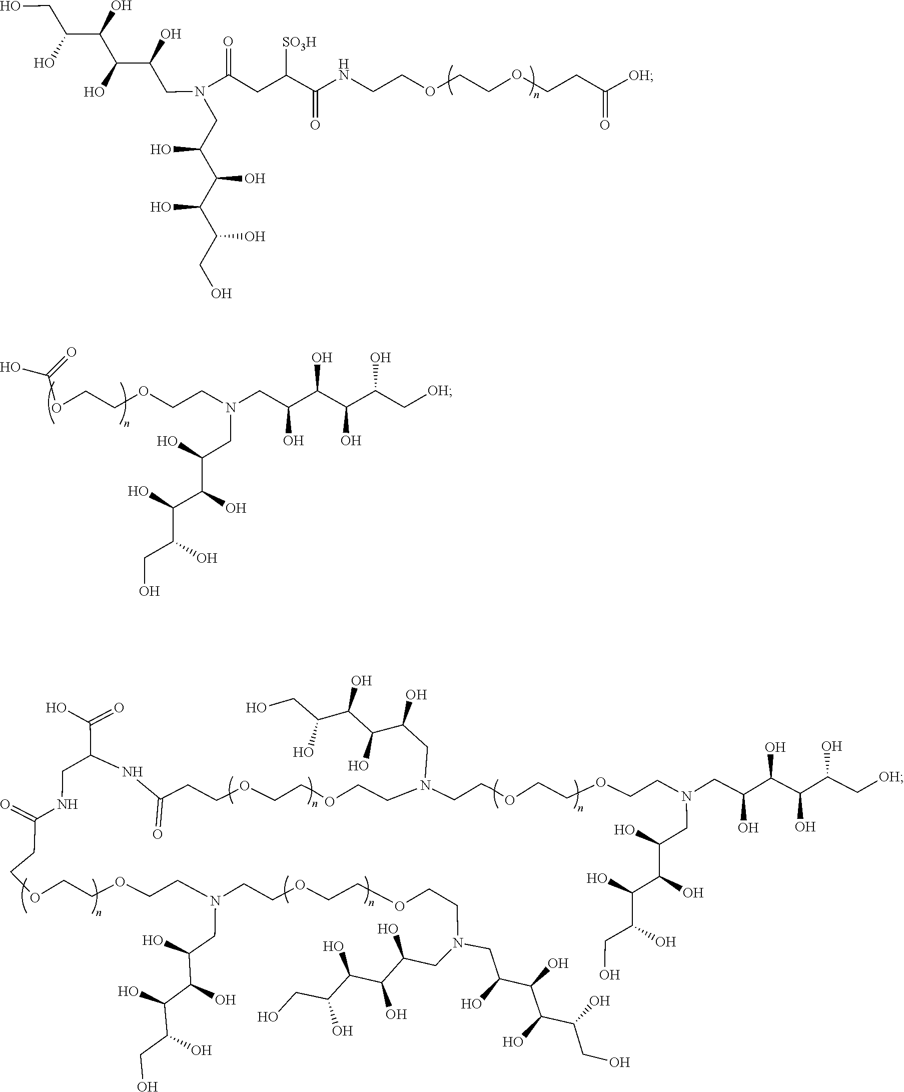

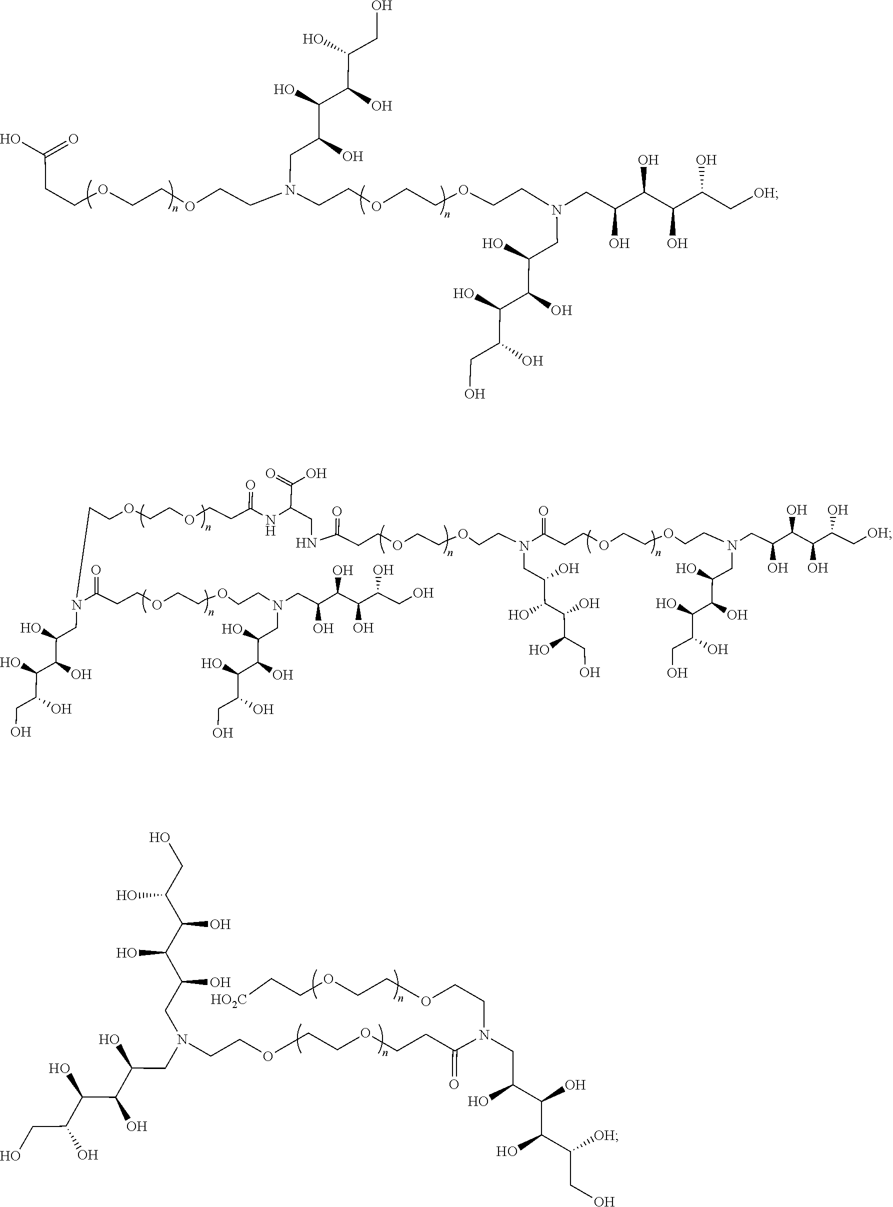

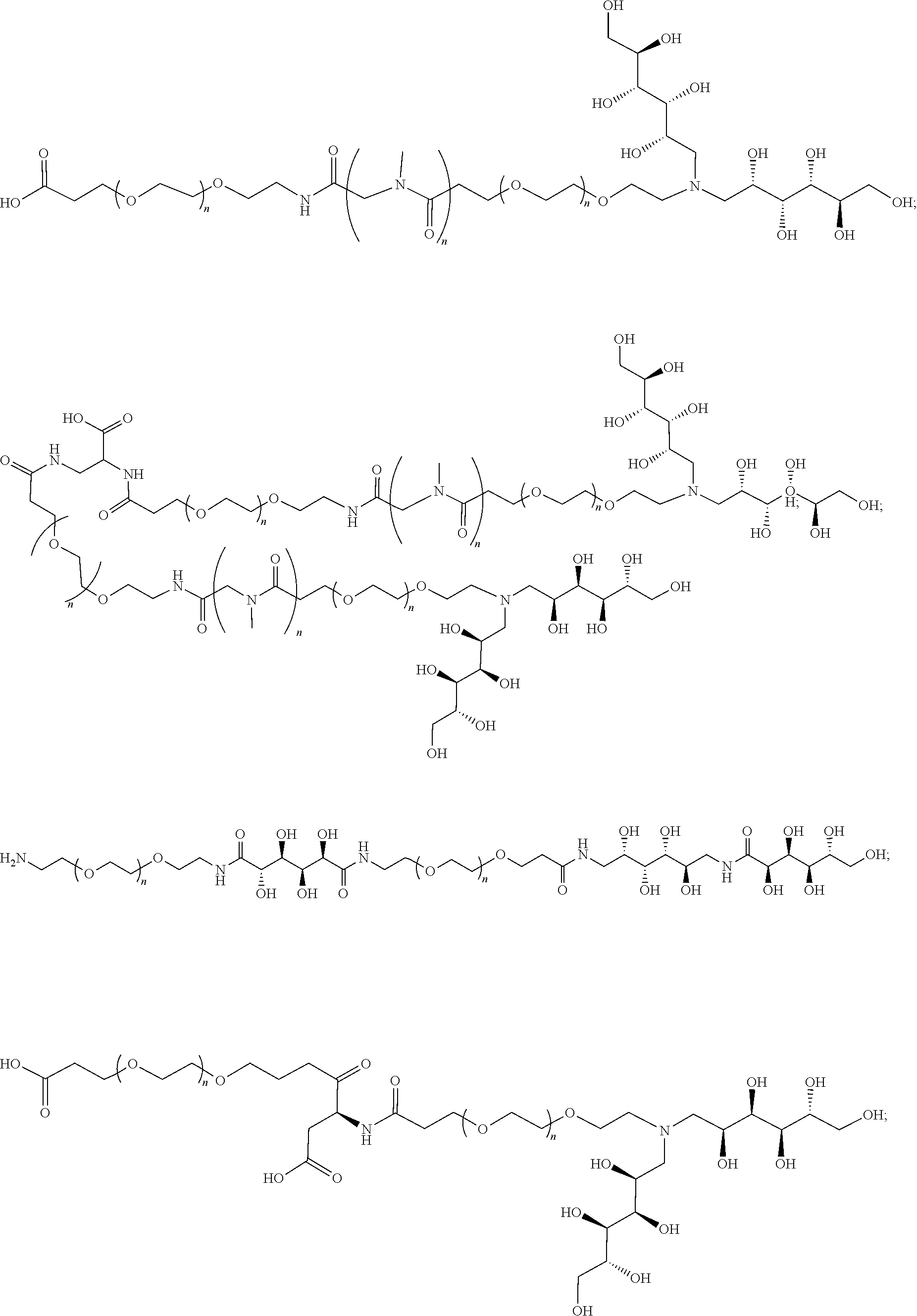

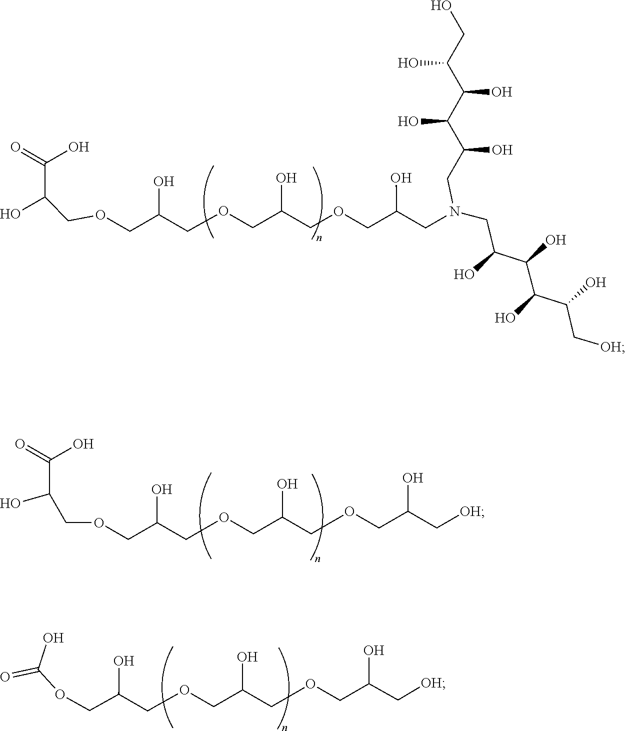

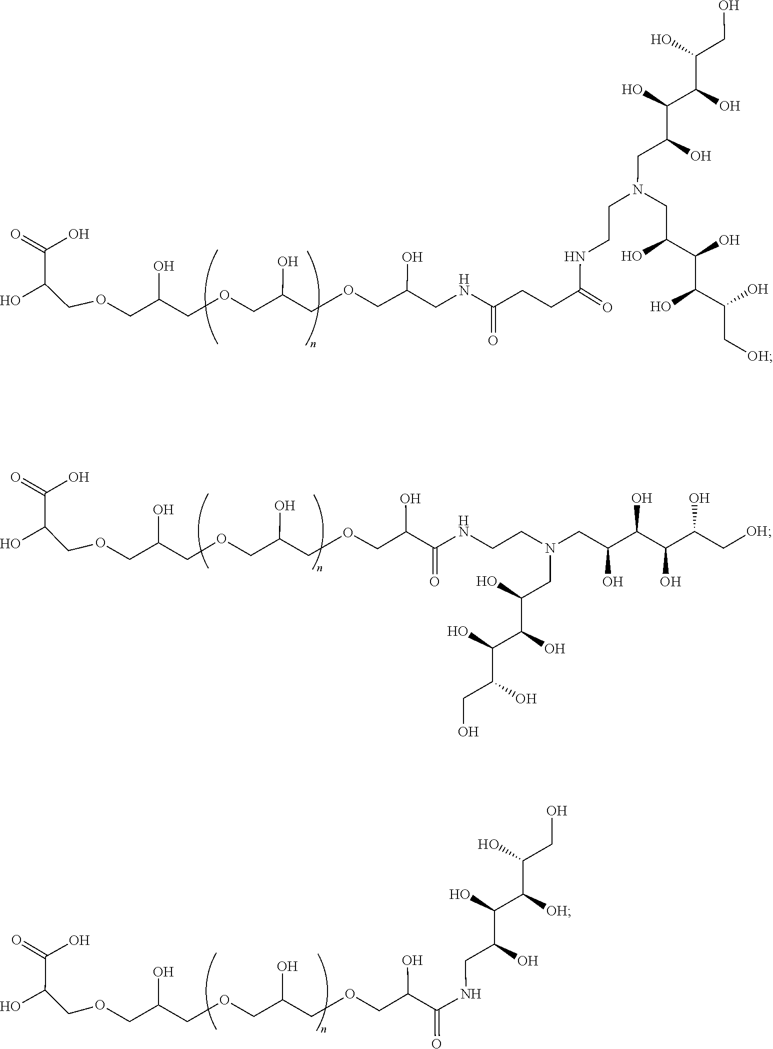

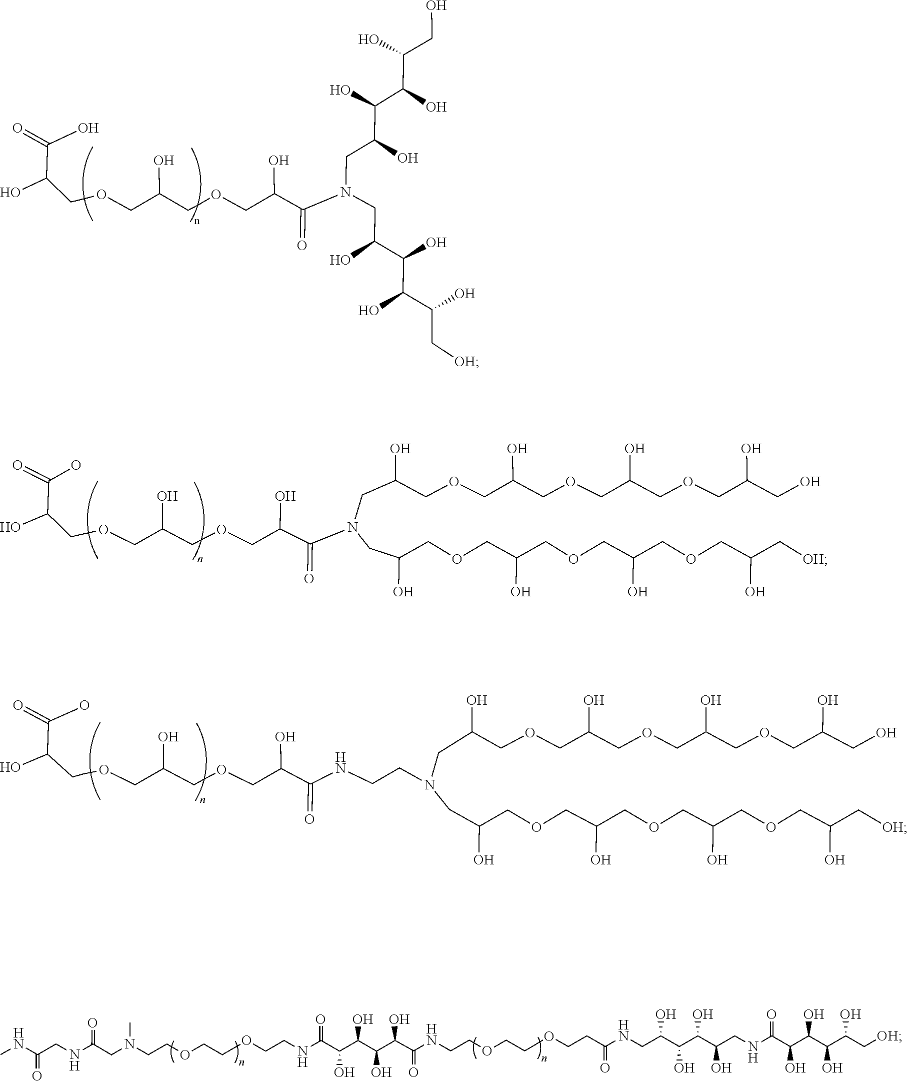

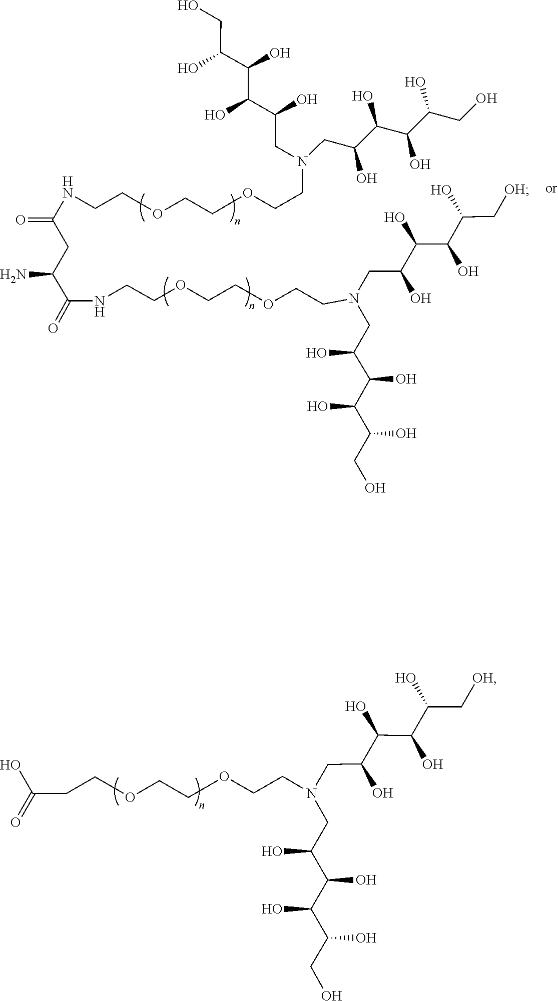

or a stereoisomer thereof, wherein R^{a} is H or C₁₋₆ alkyl and n is 1-20.

Embodiment 76. The linker compound of any one of Embodiments 1-75, the polar group is selected from the following, or a stereoisomer or salt thereof:

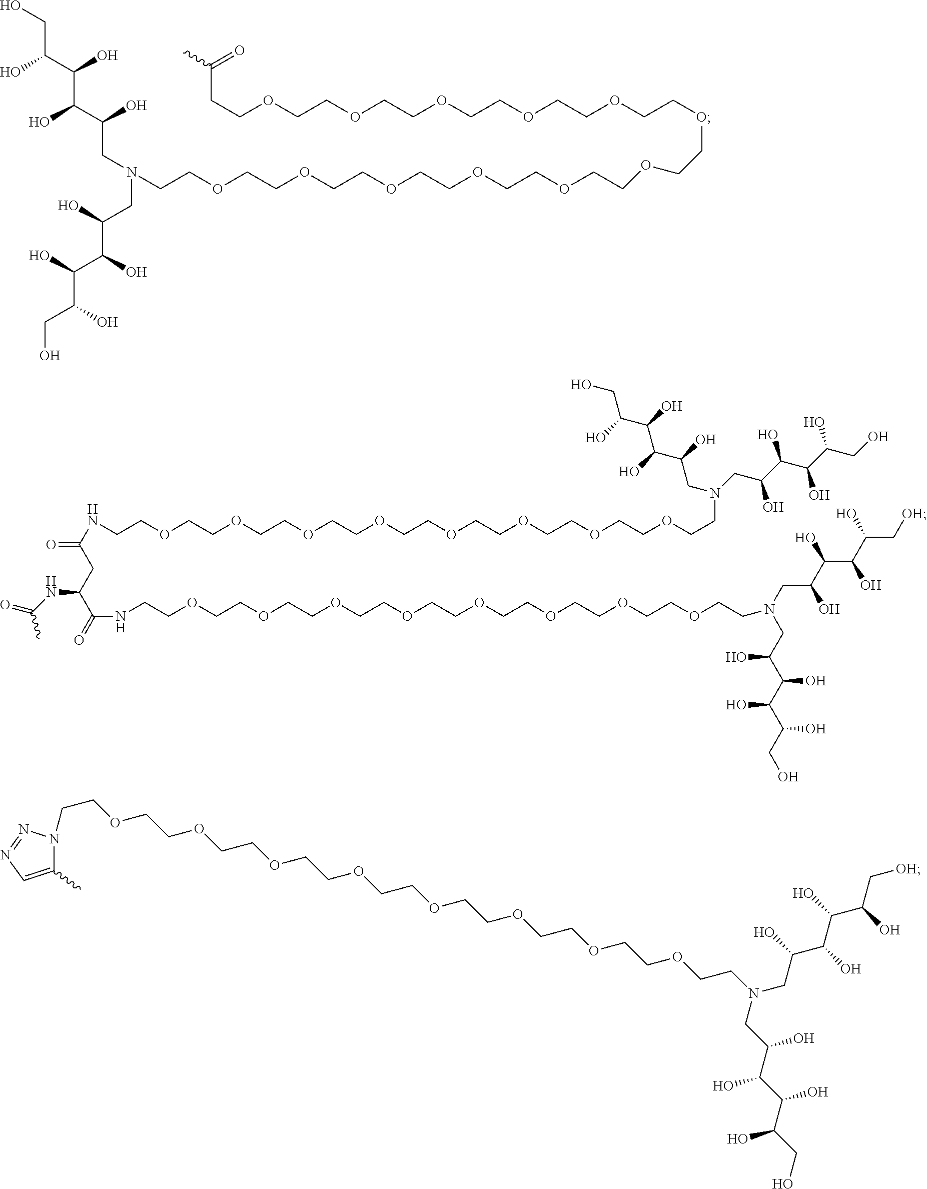

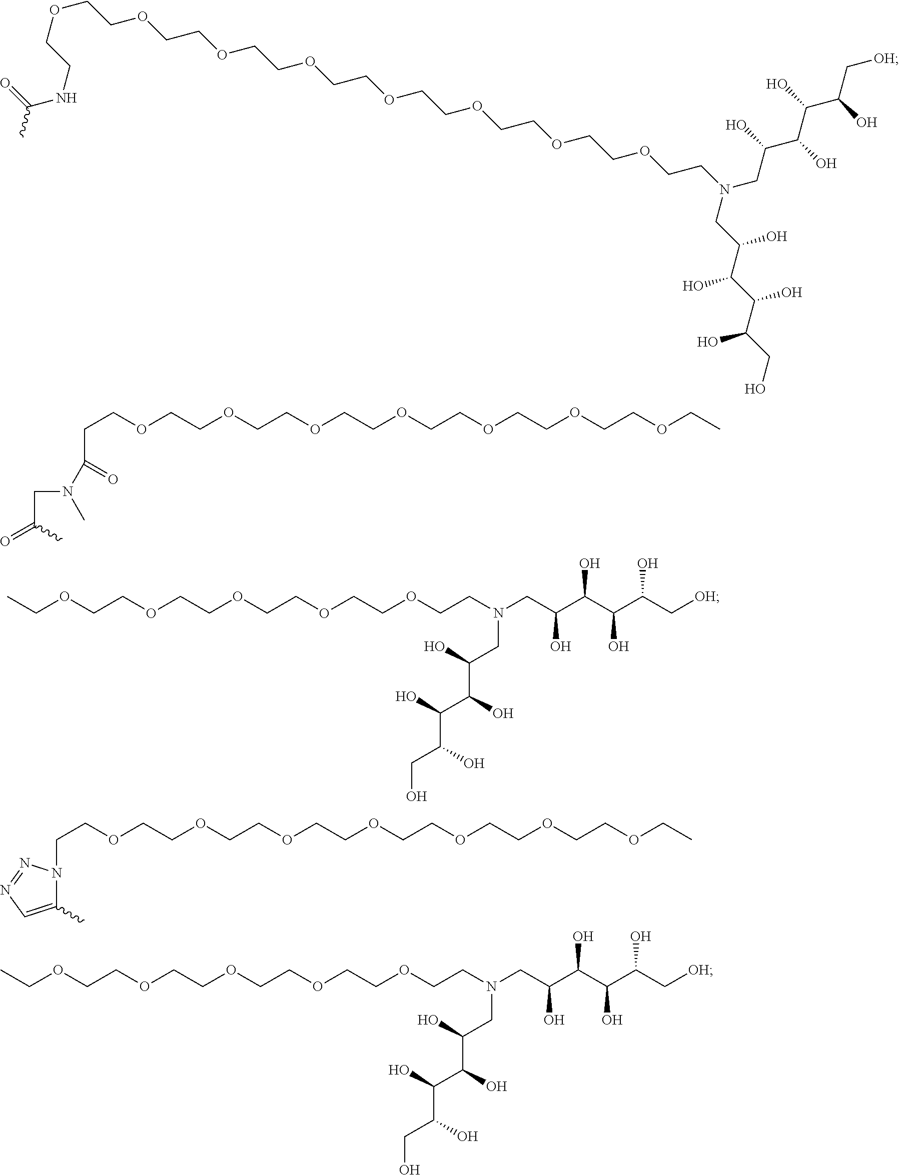

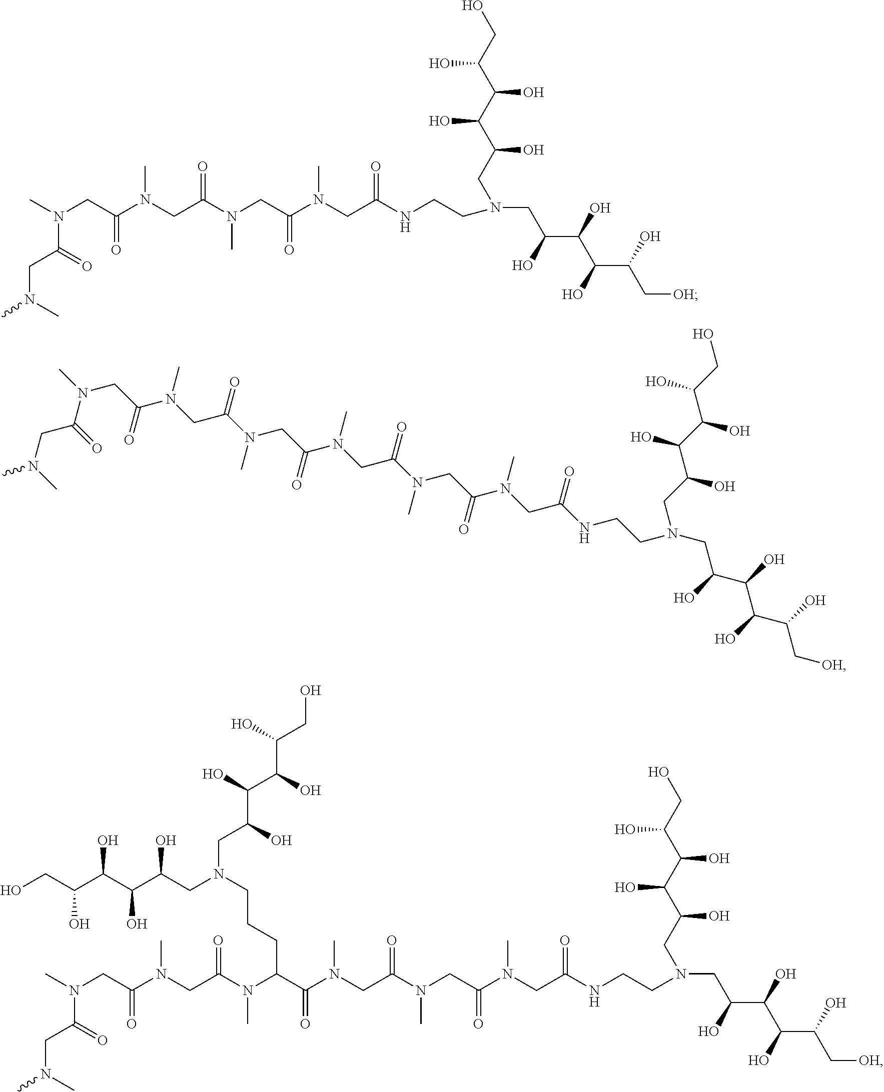

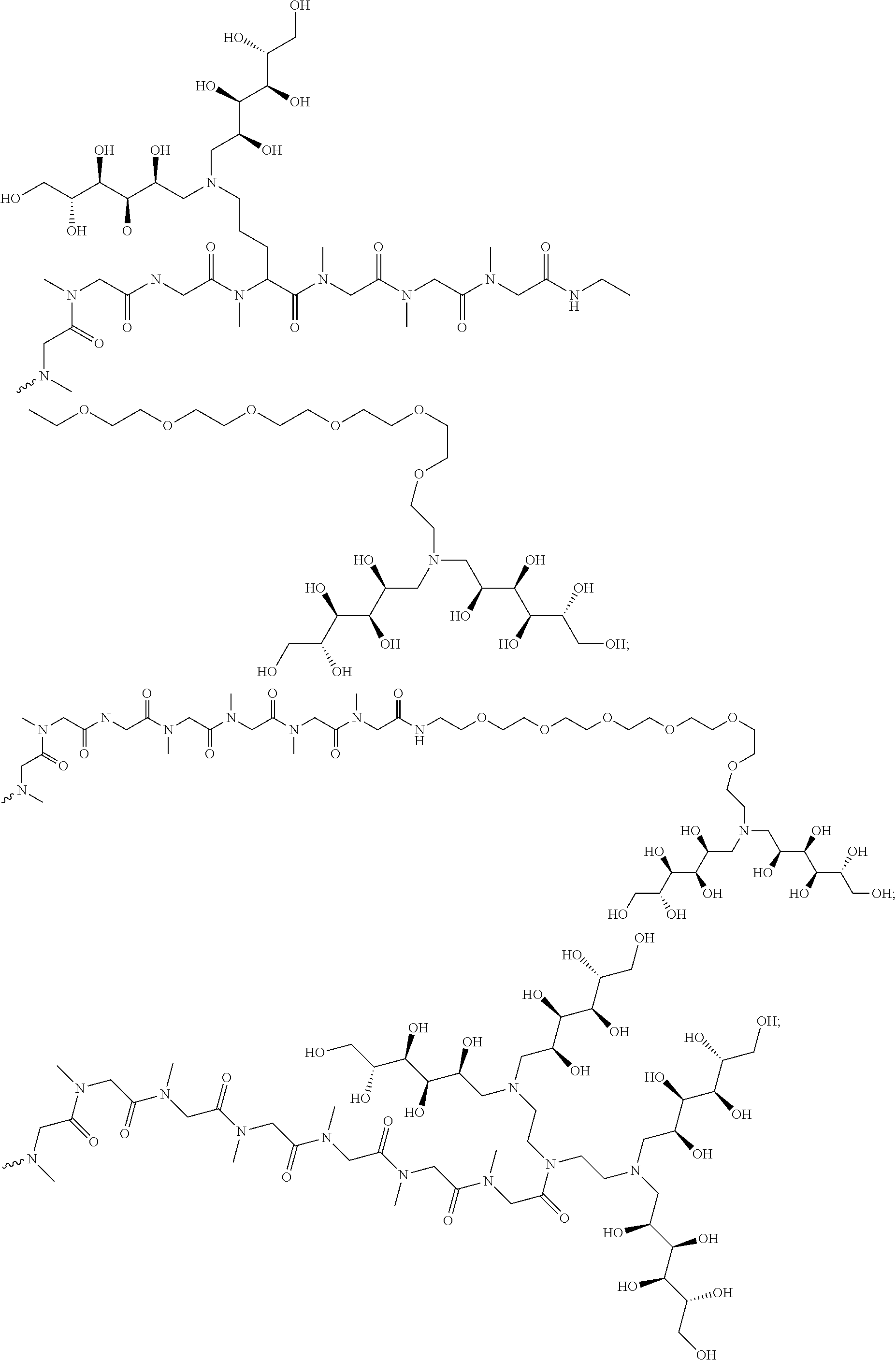

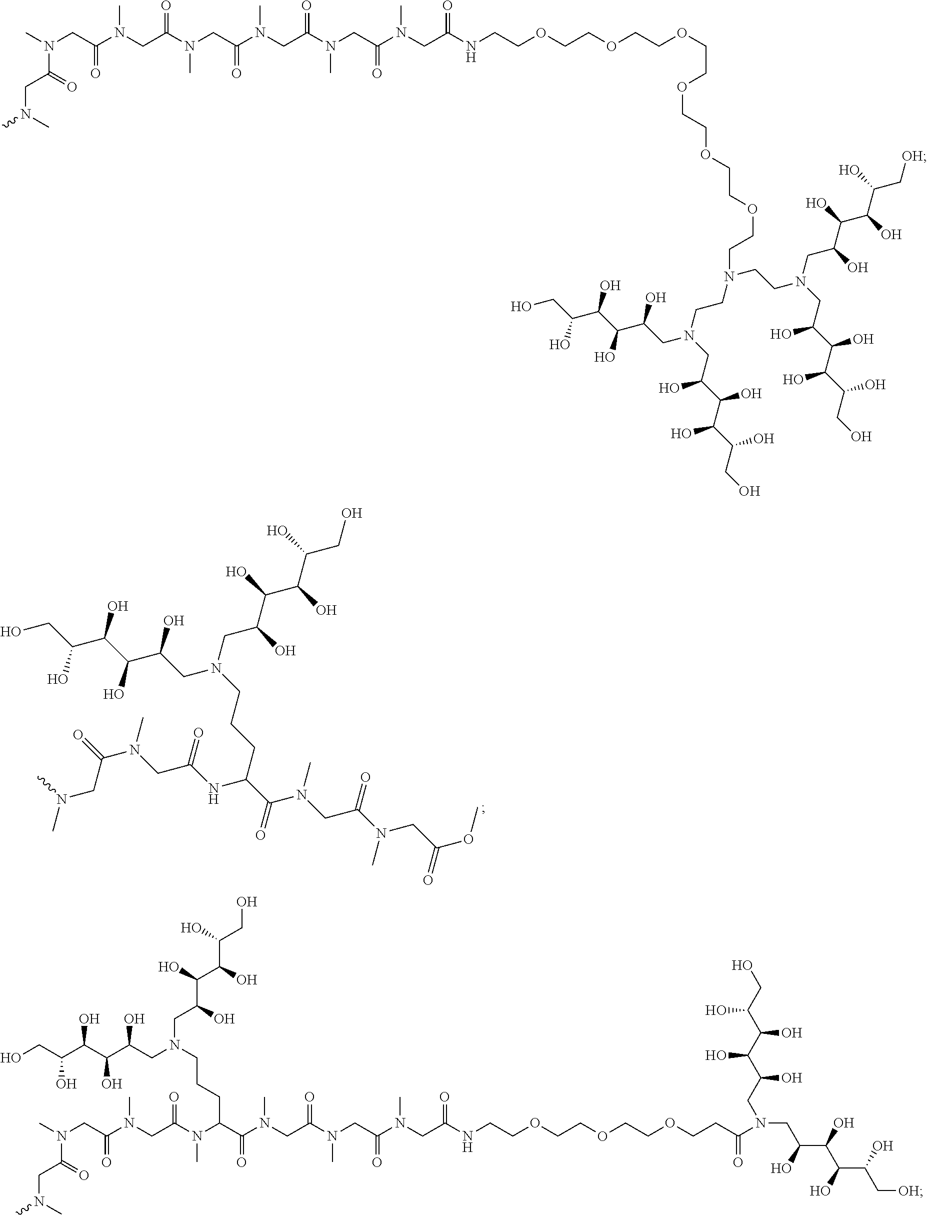

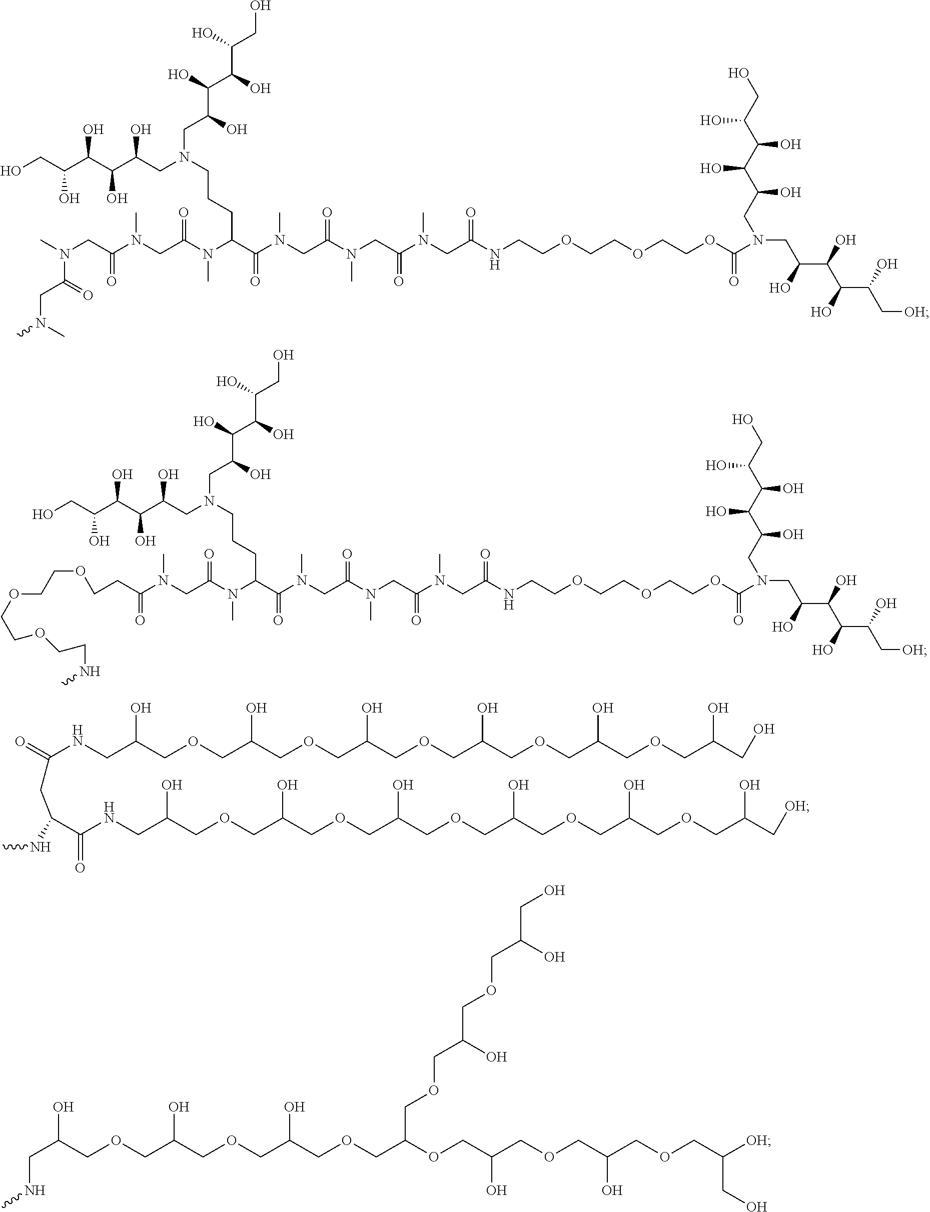

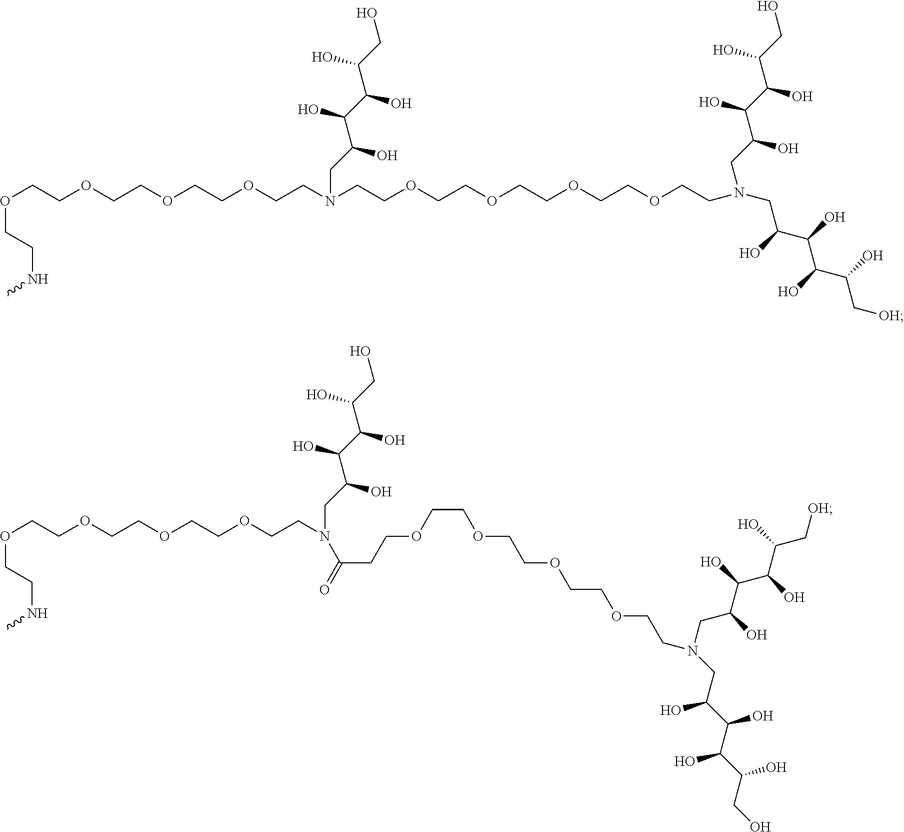

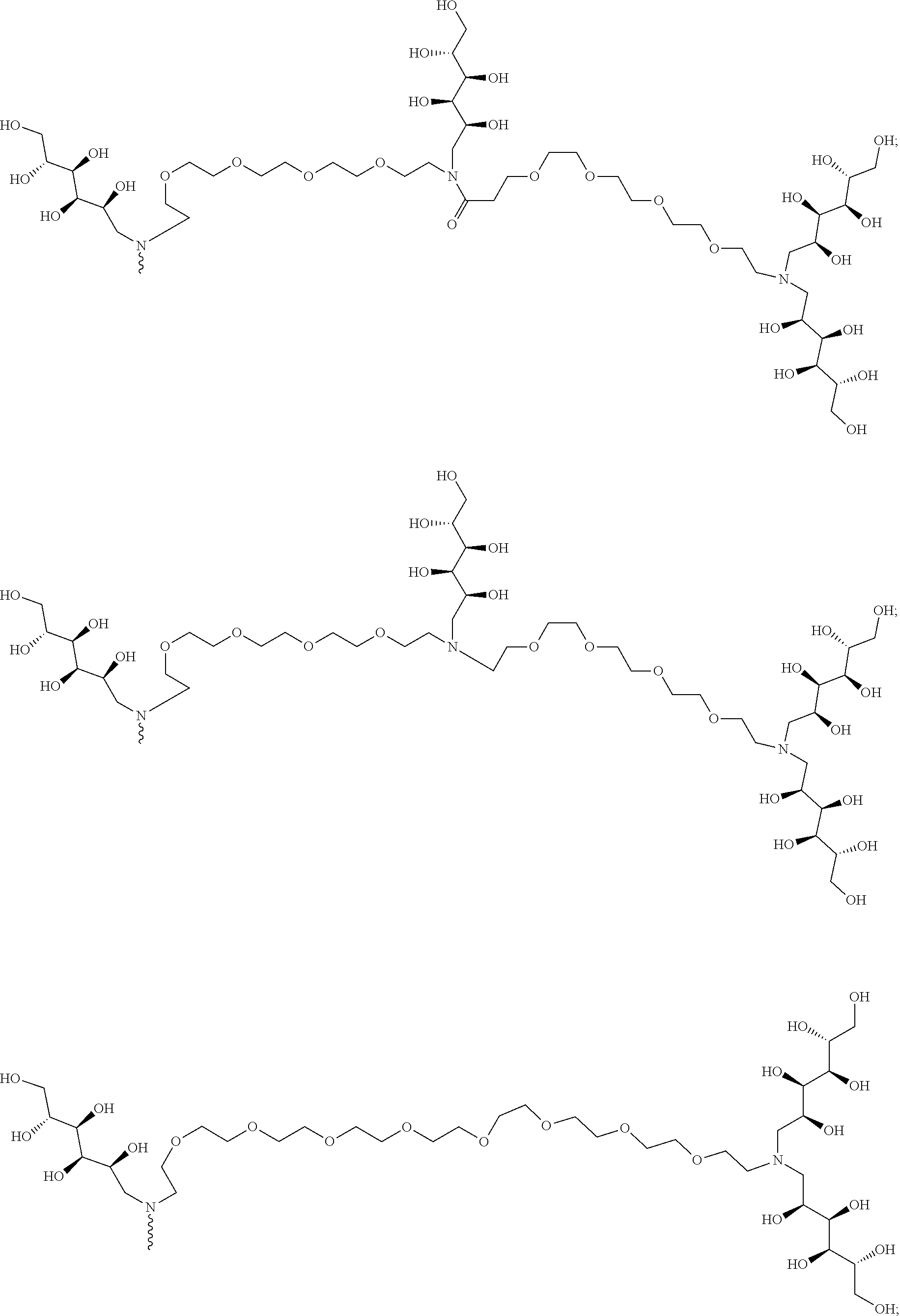

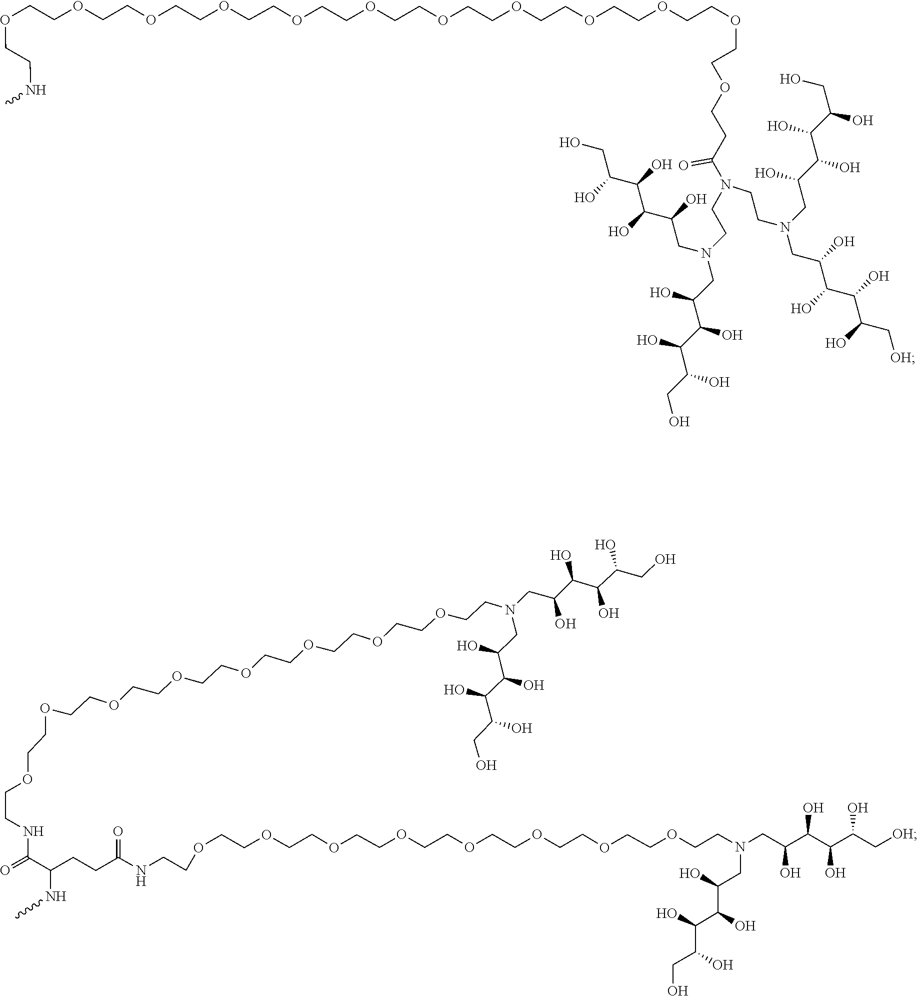

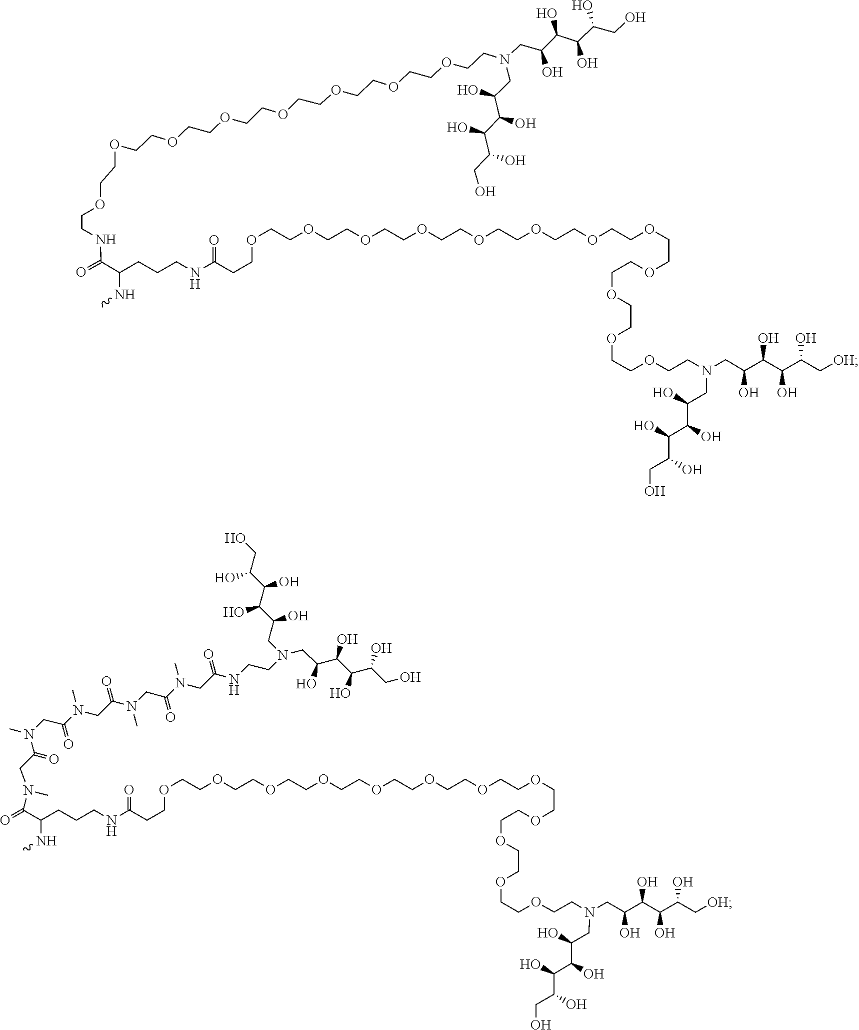

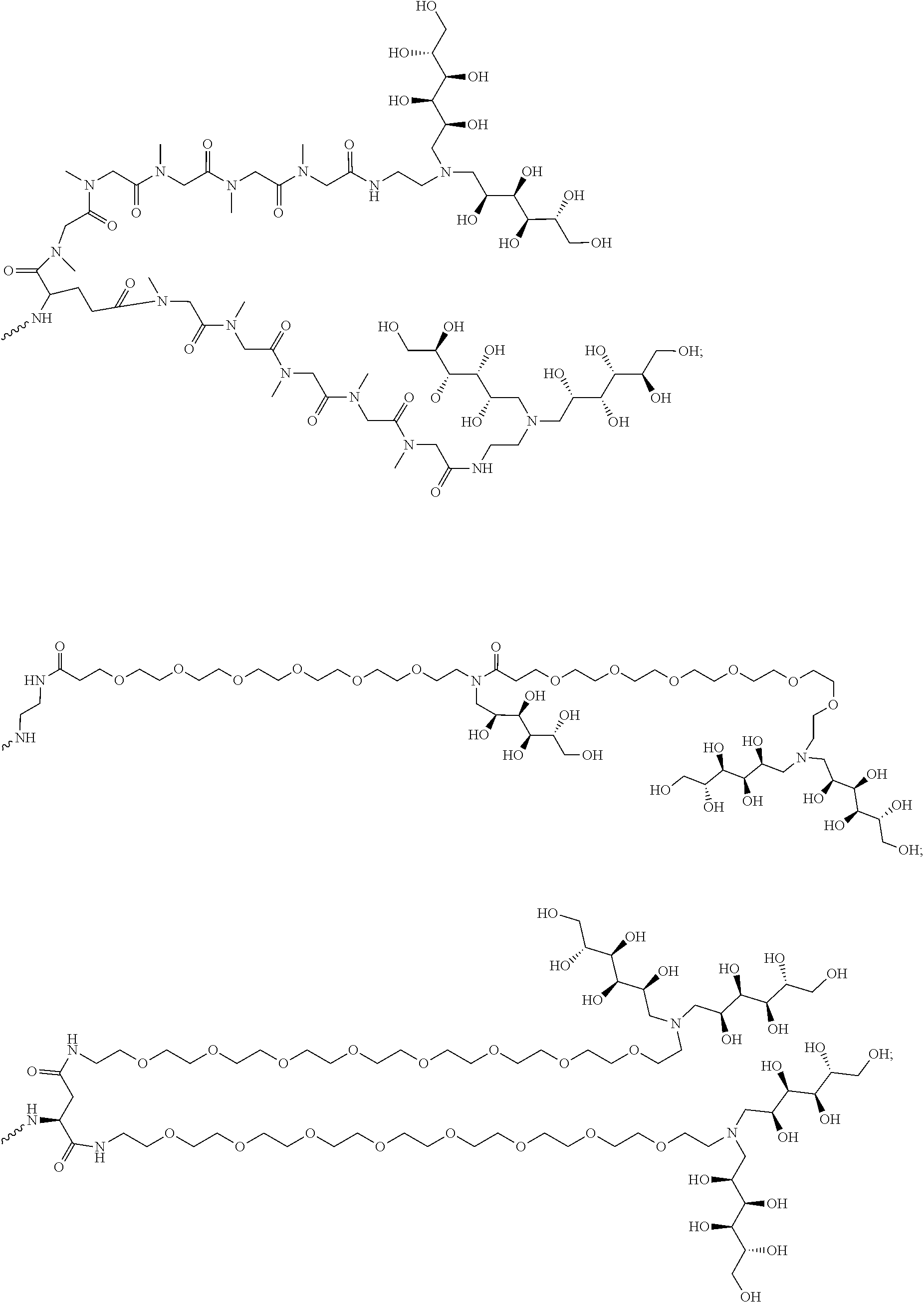

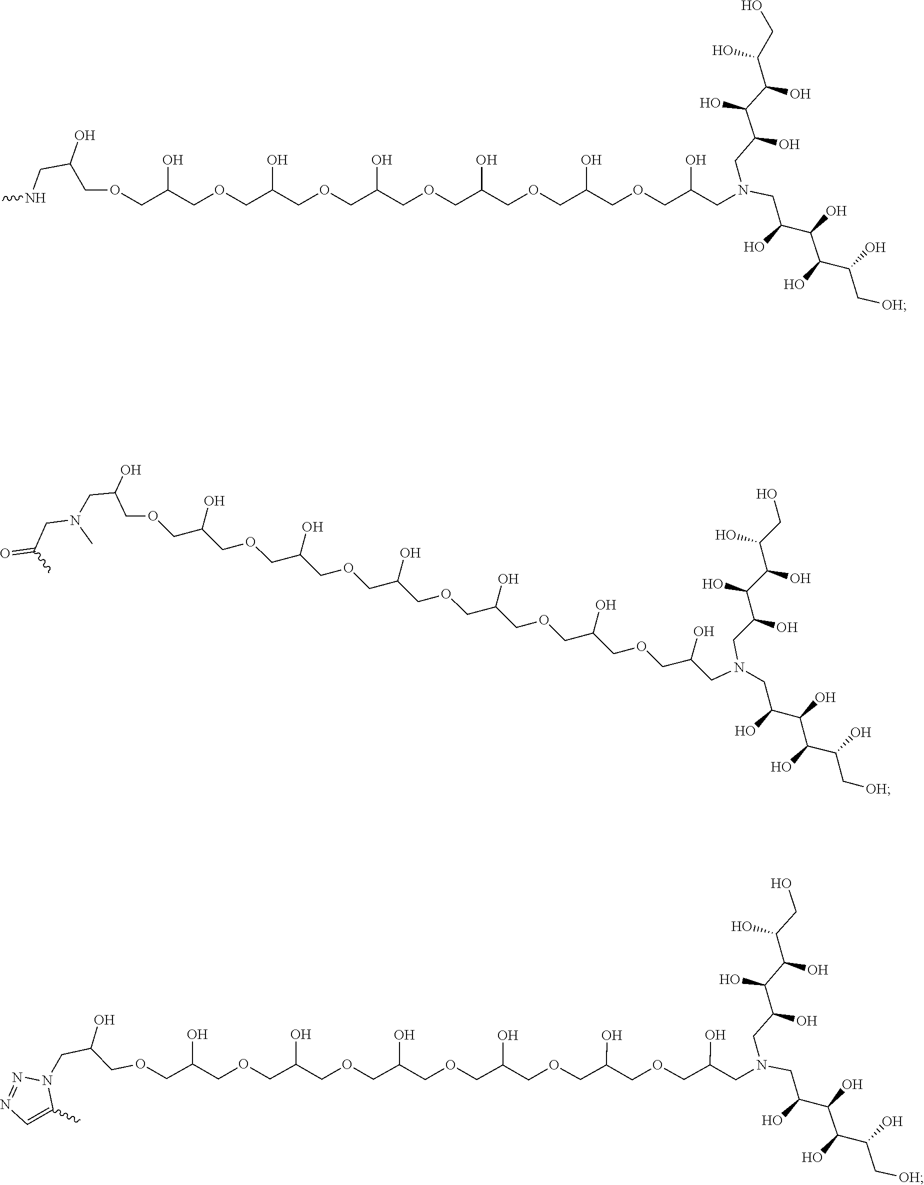

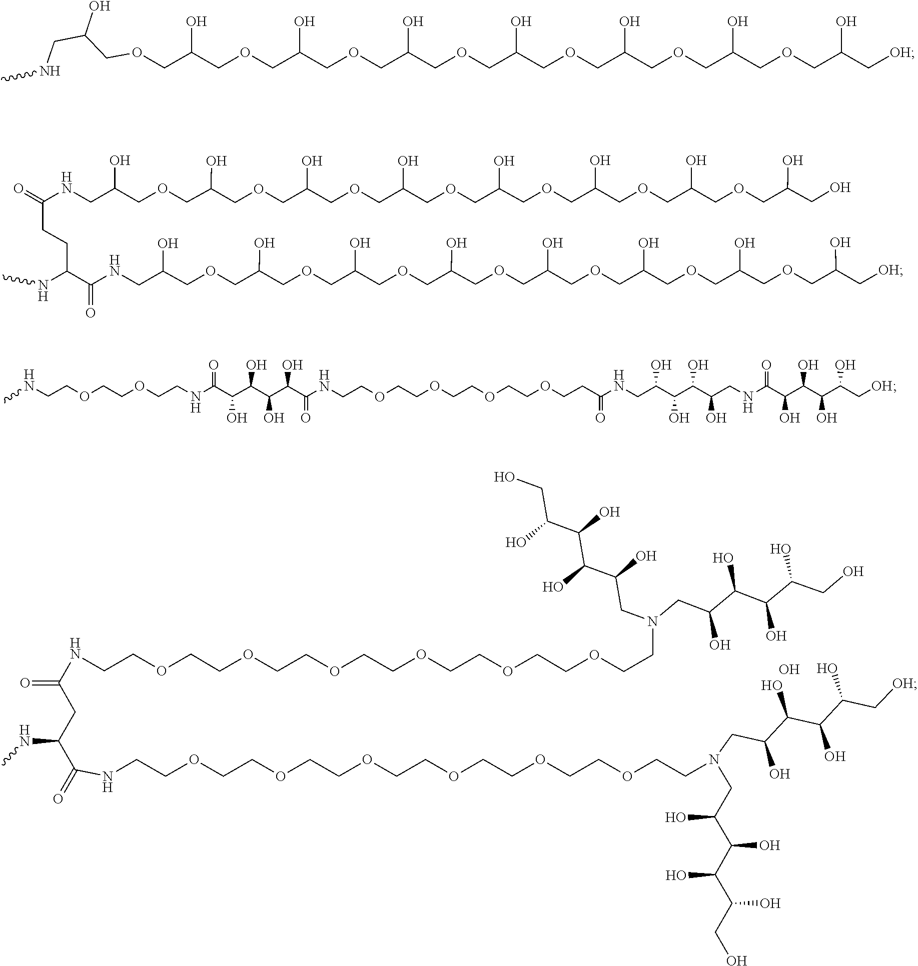

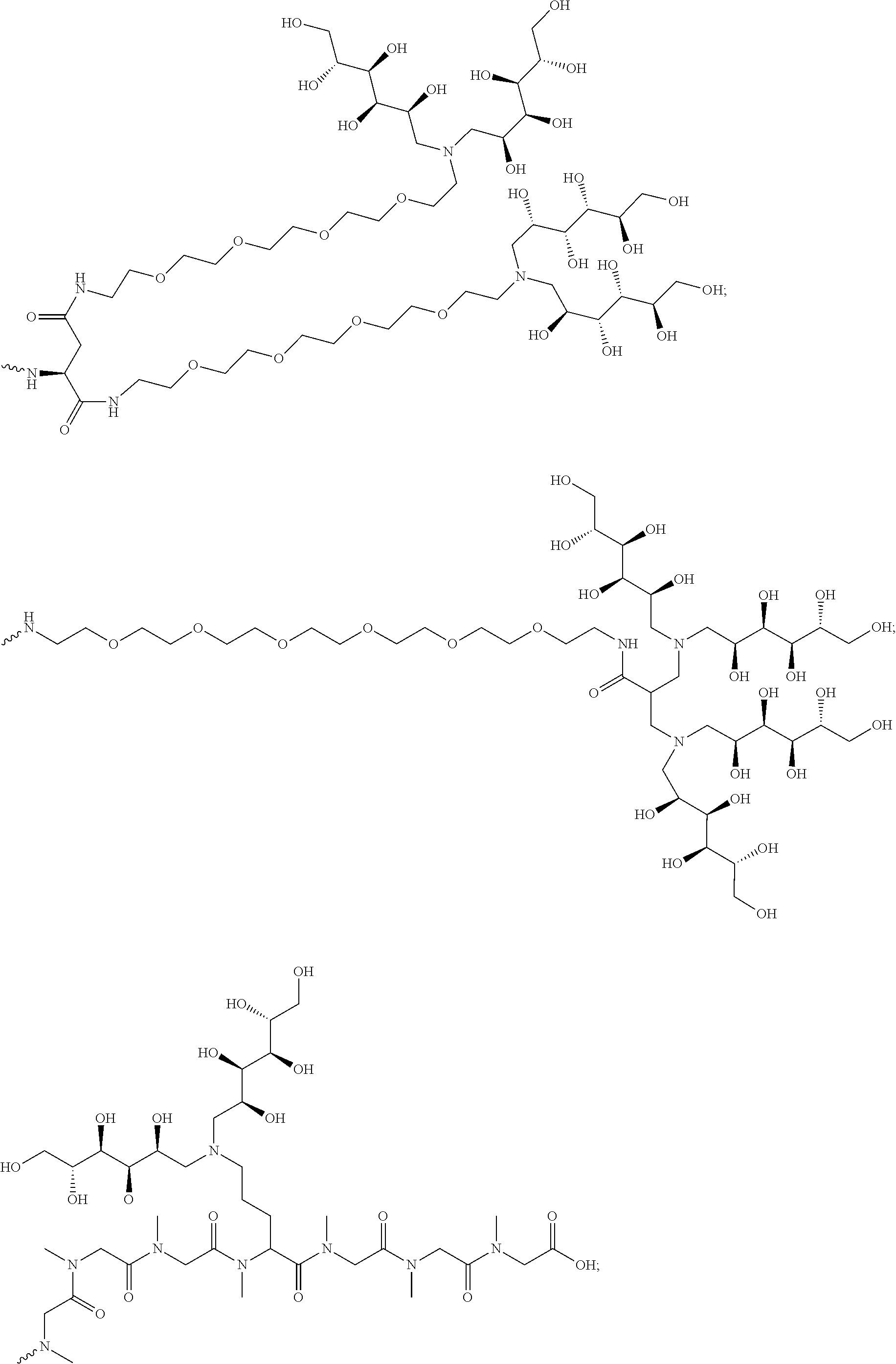

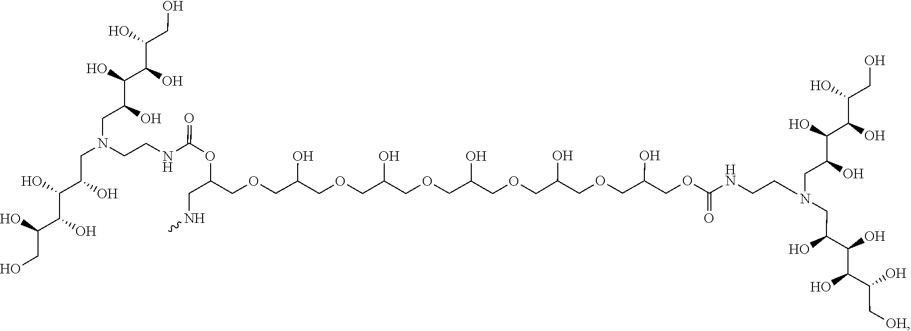

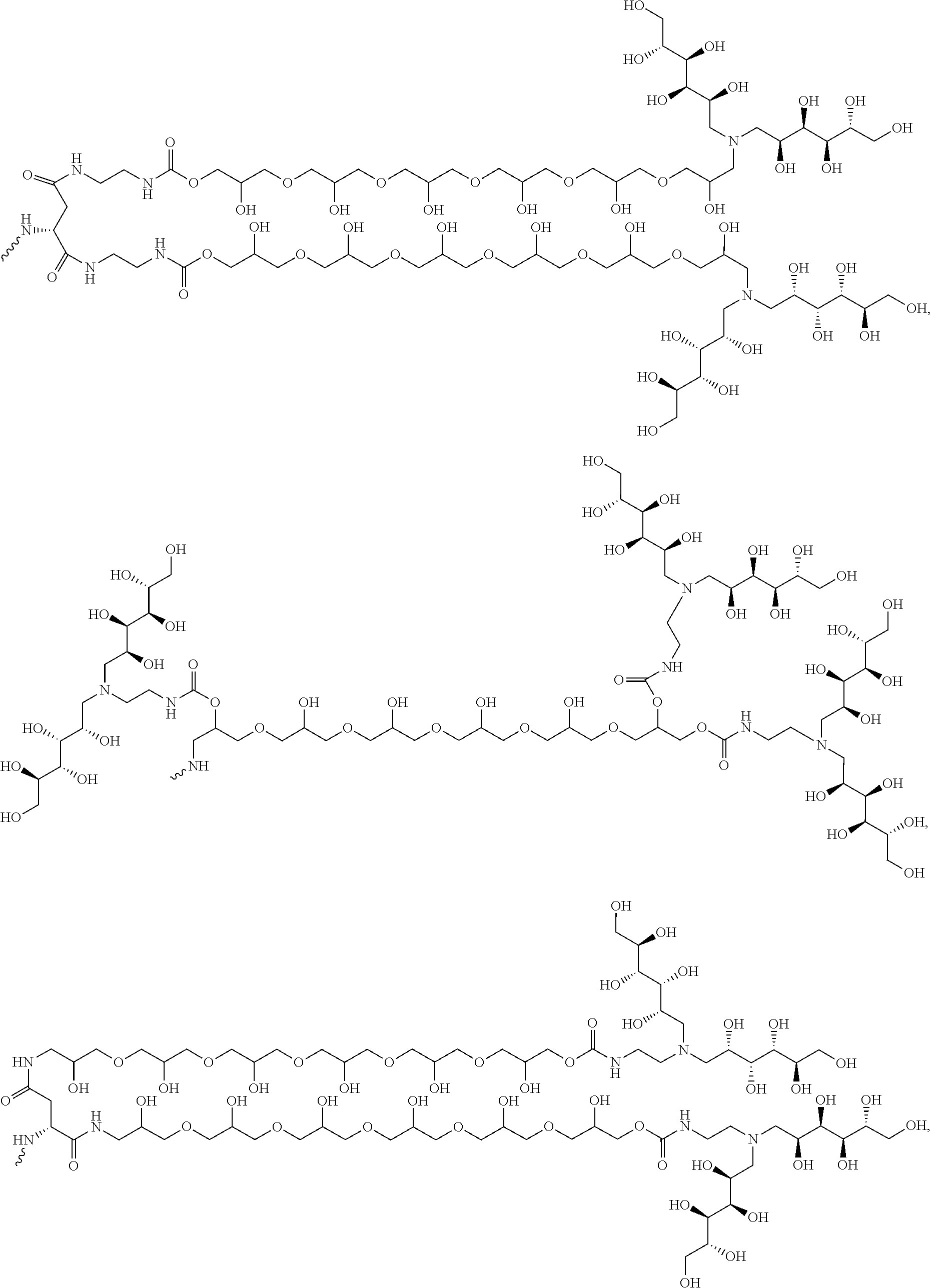

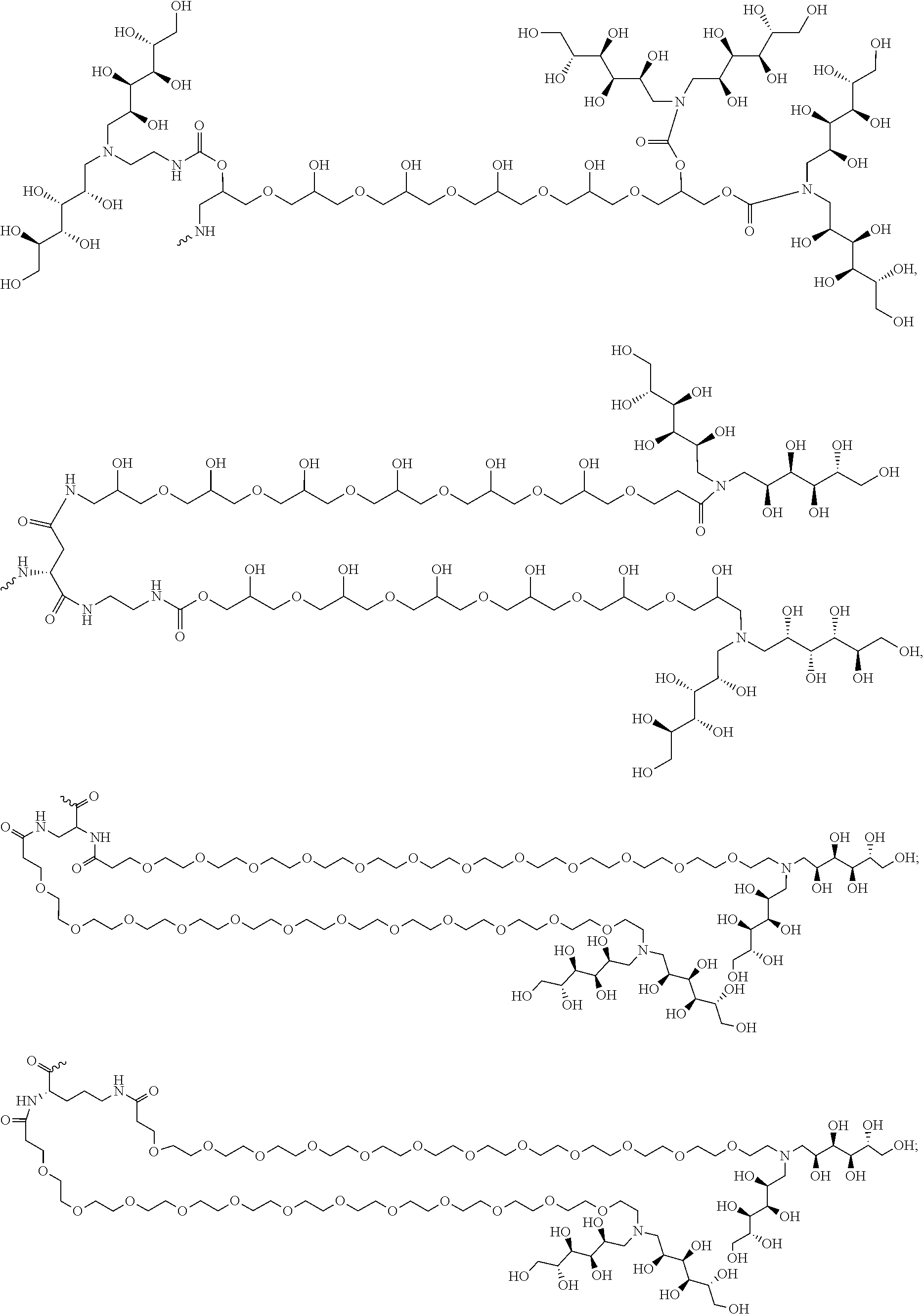

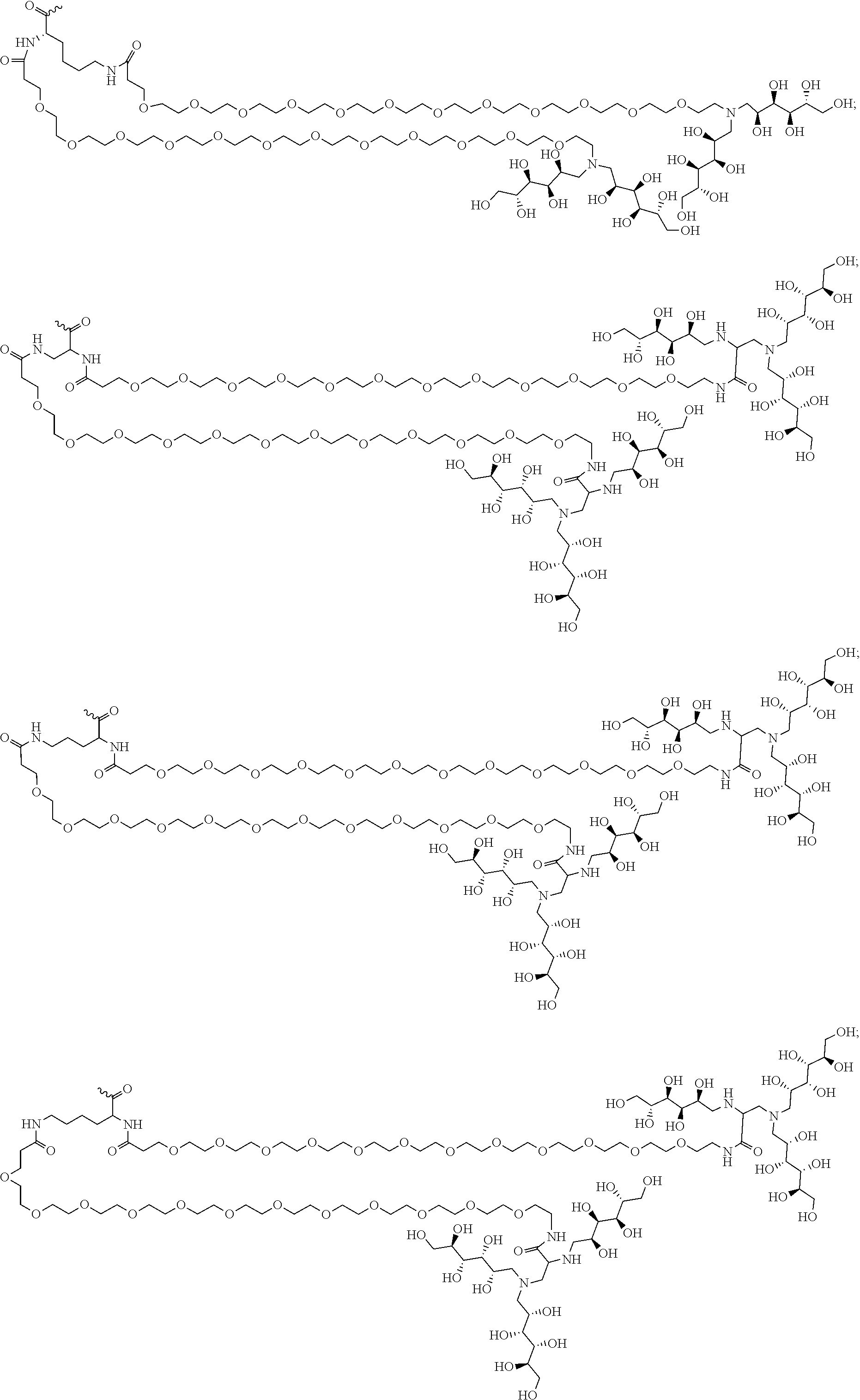

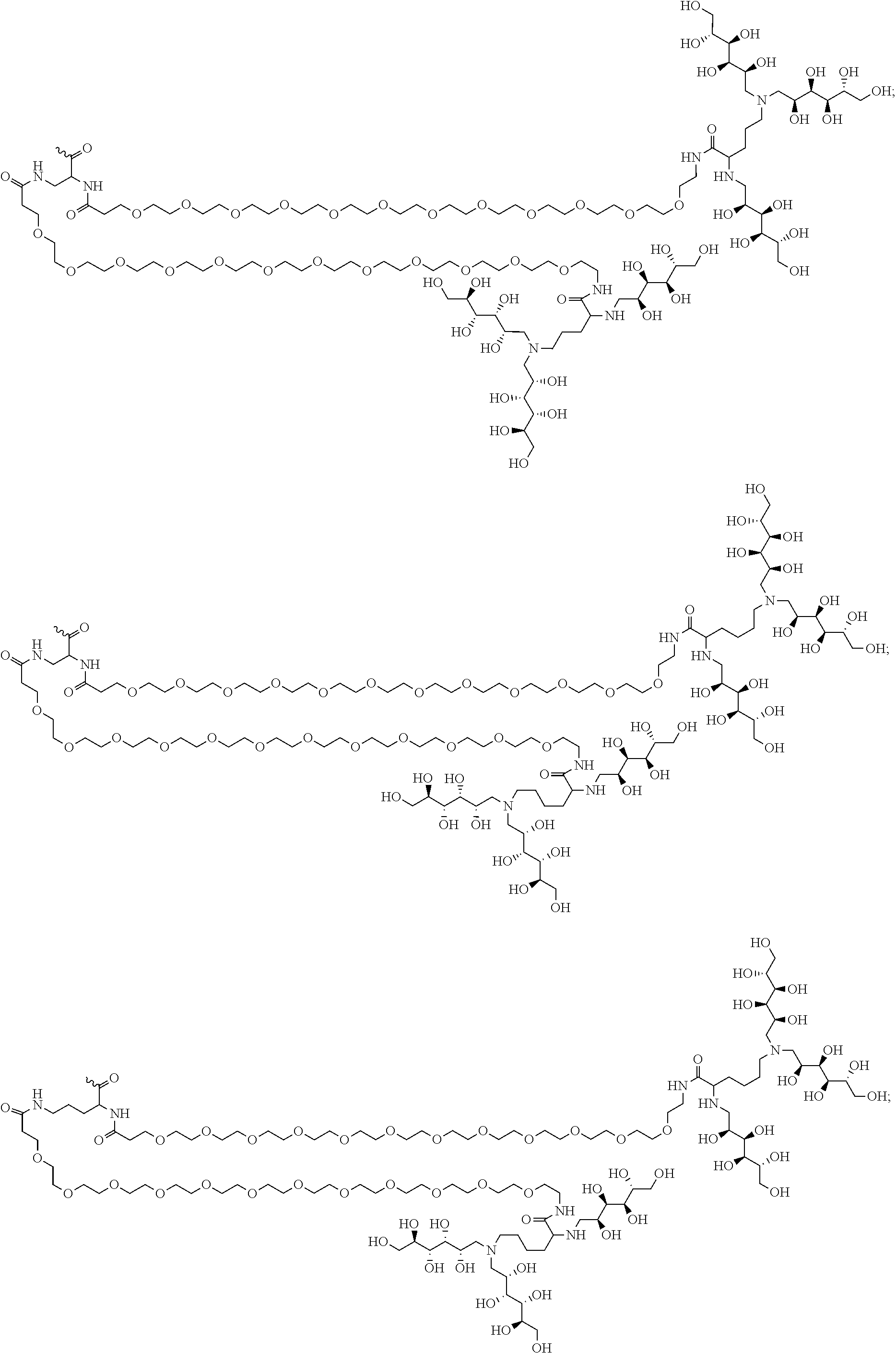

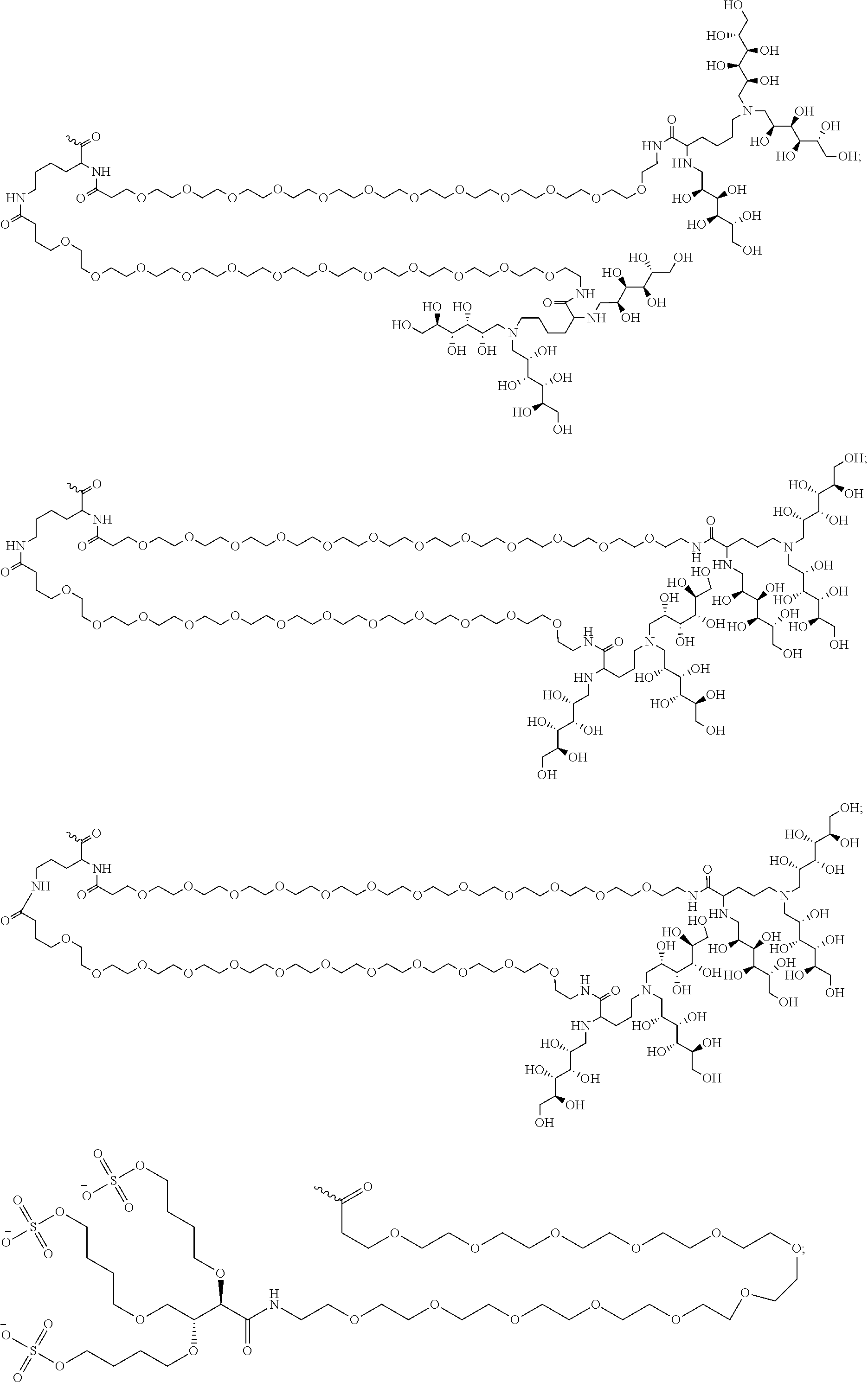

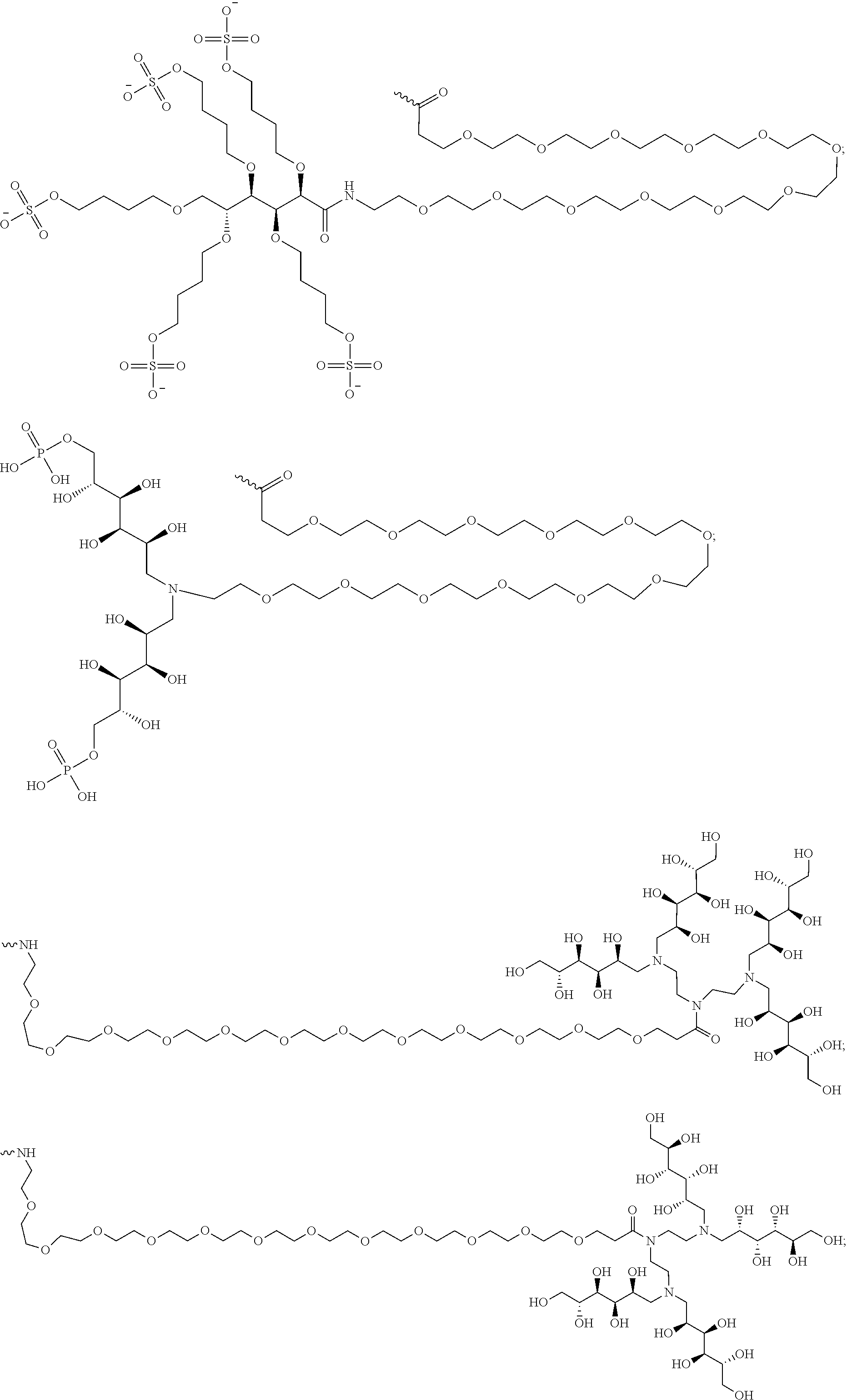

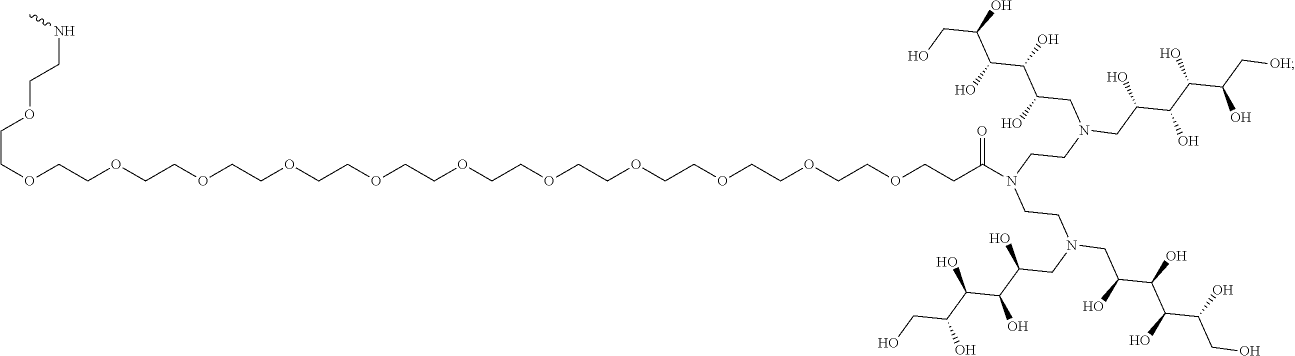

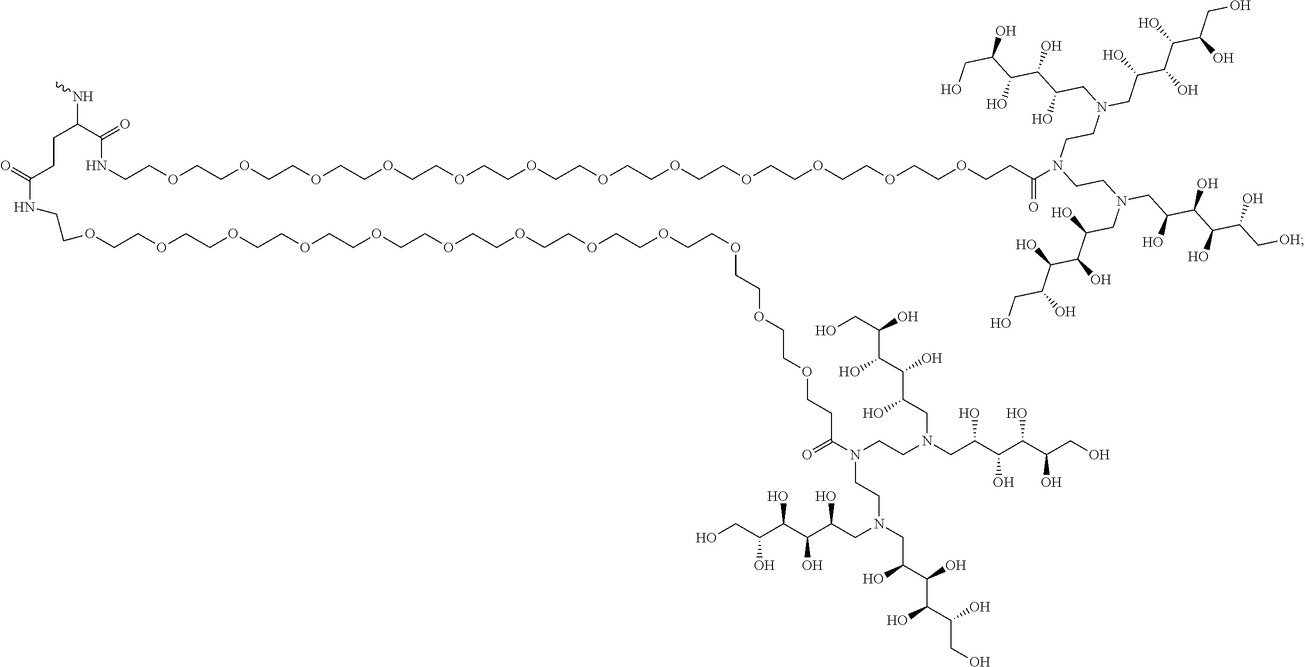

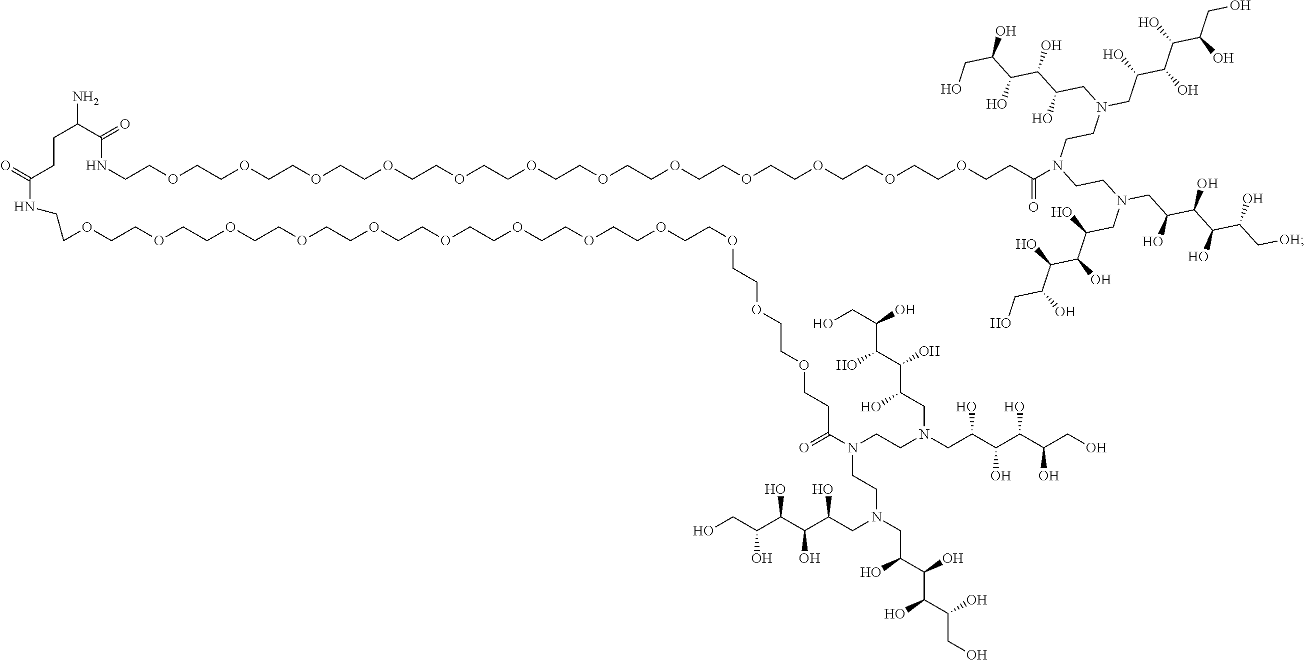

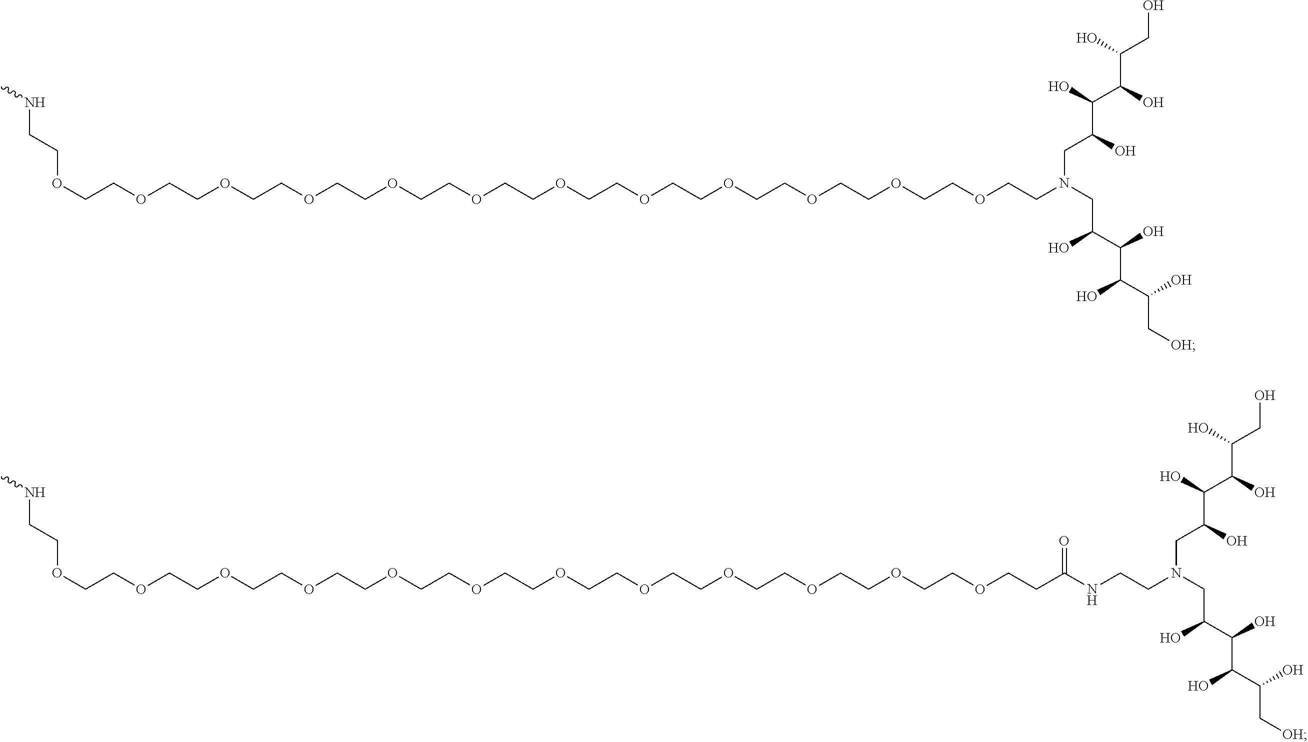

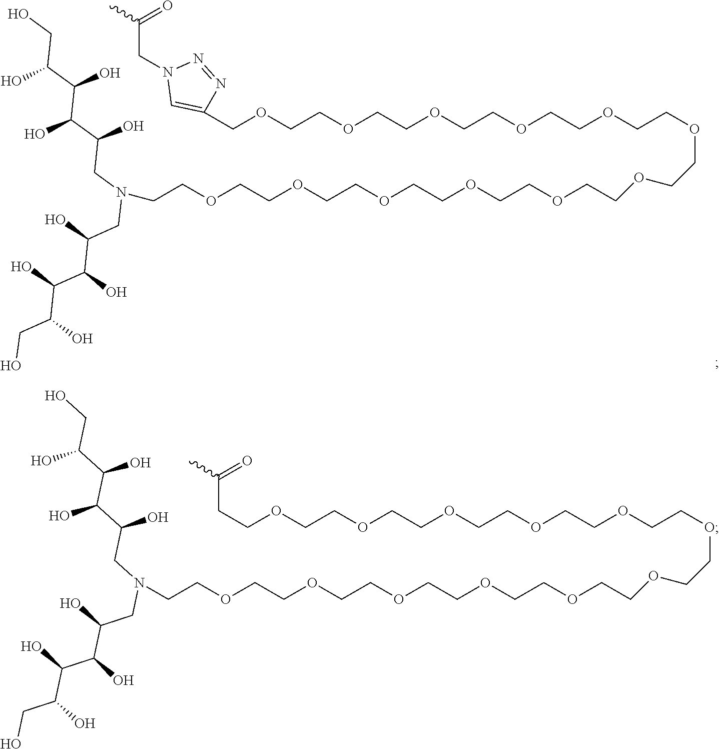

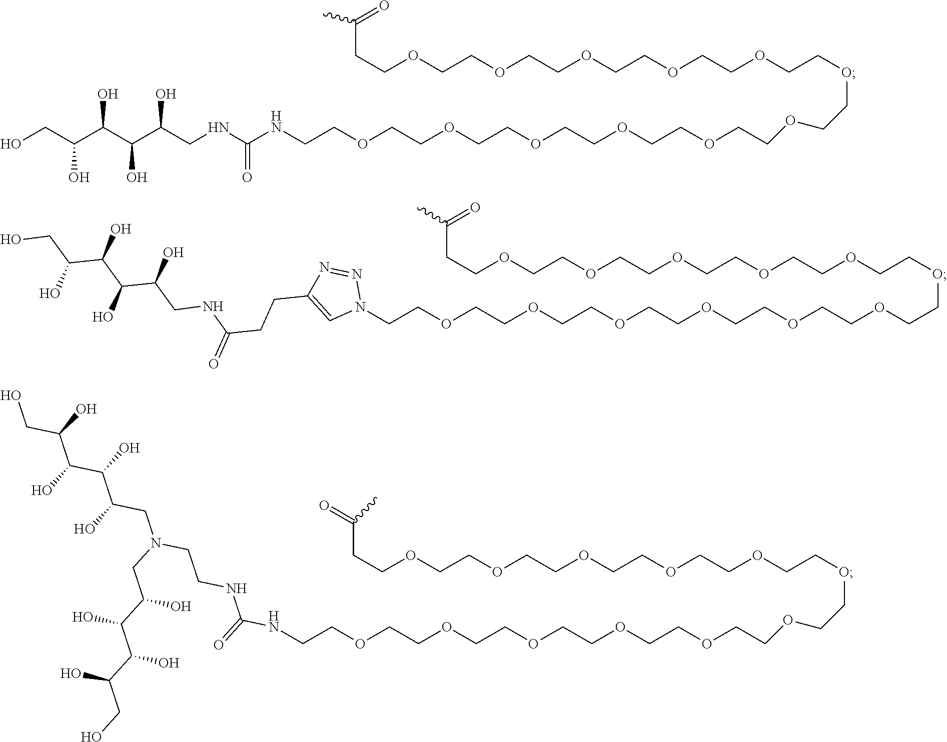

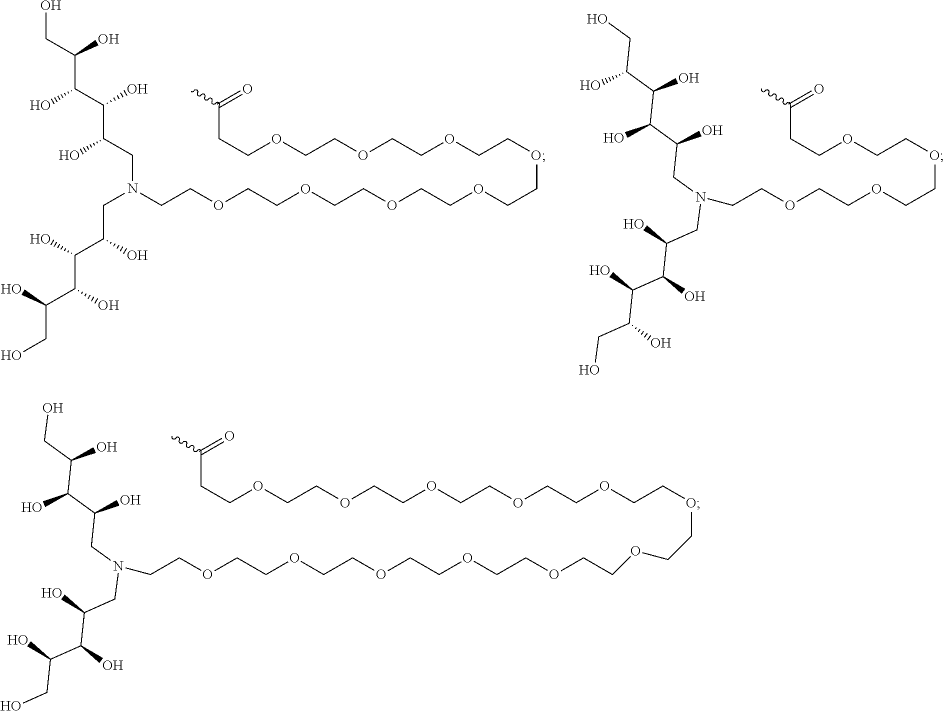

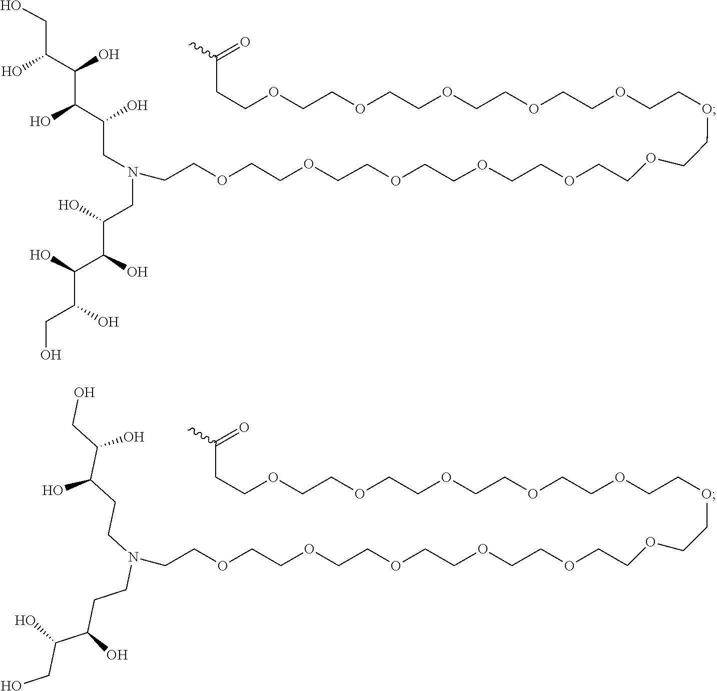

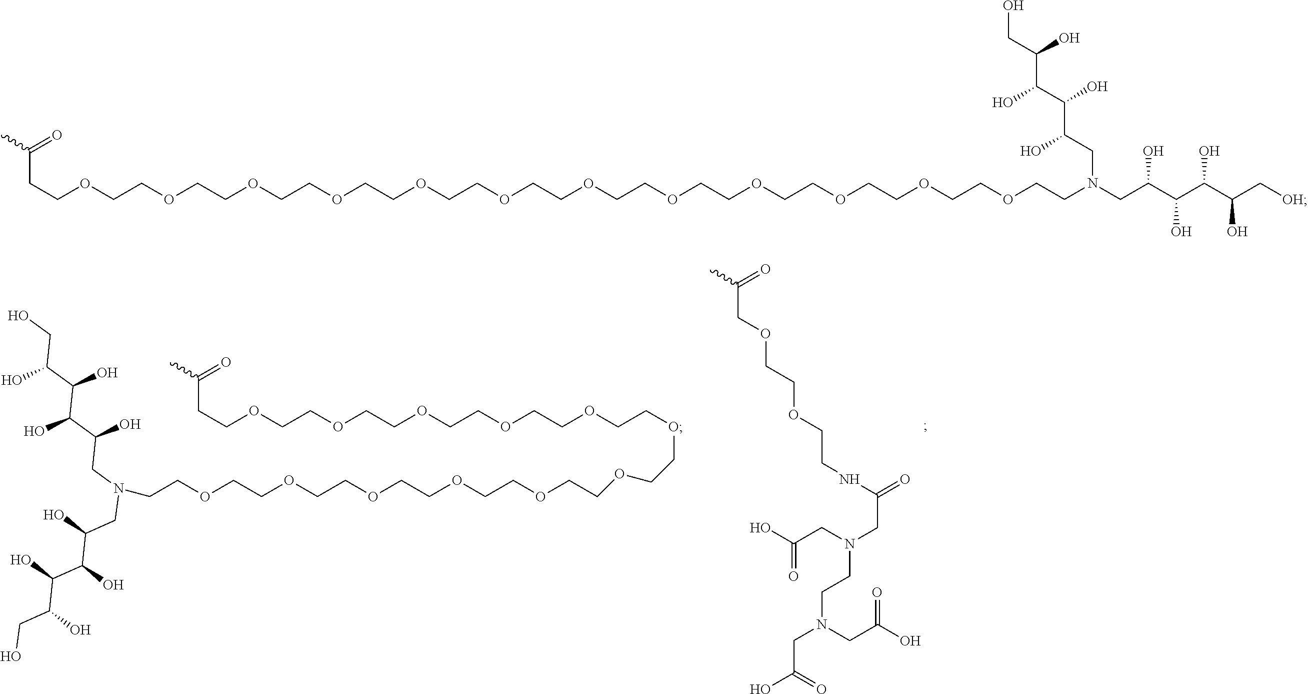

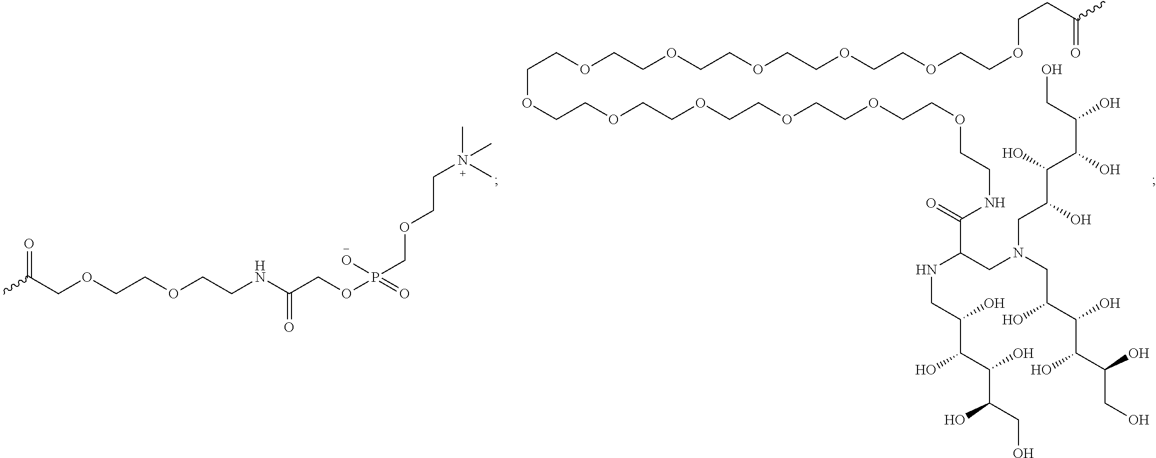

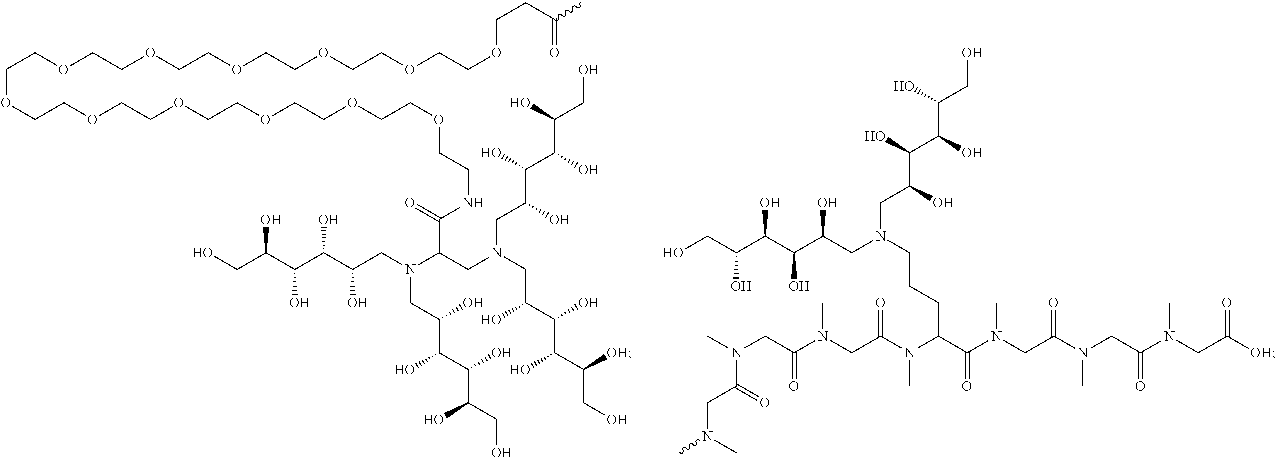

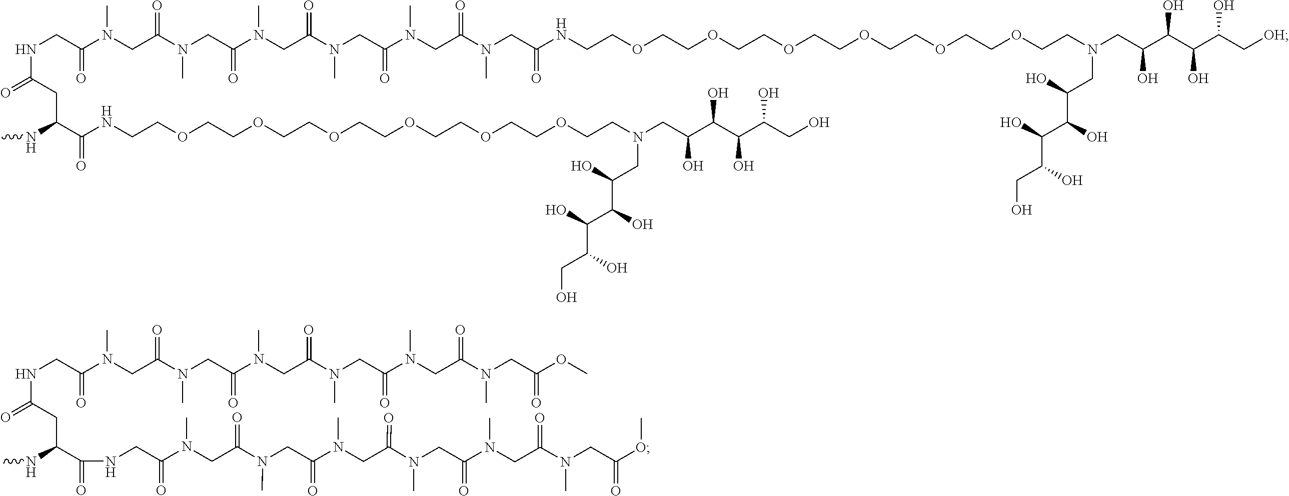

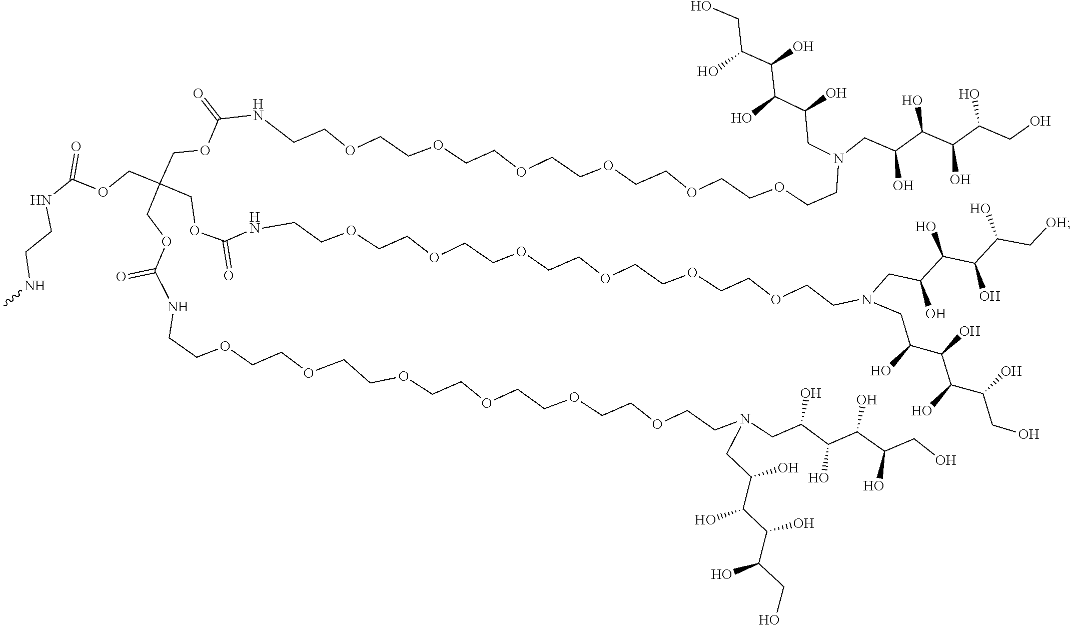

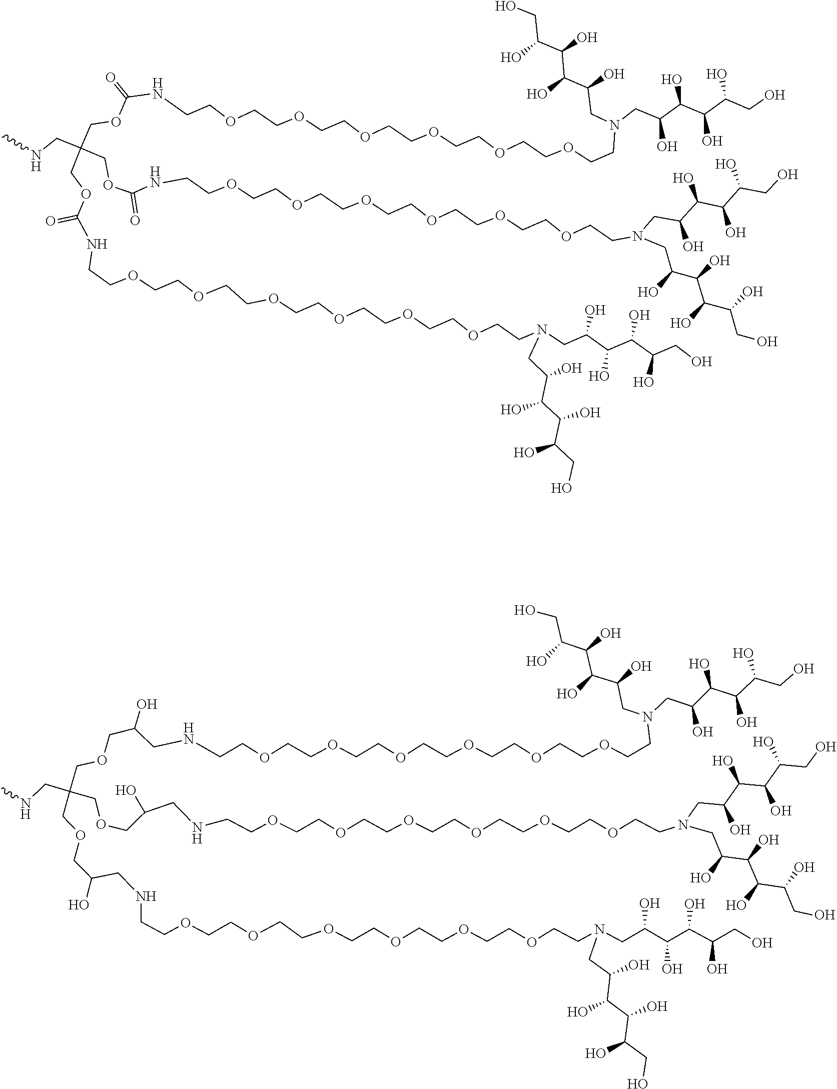

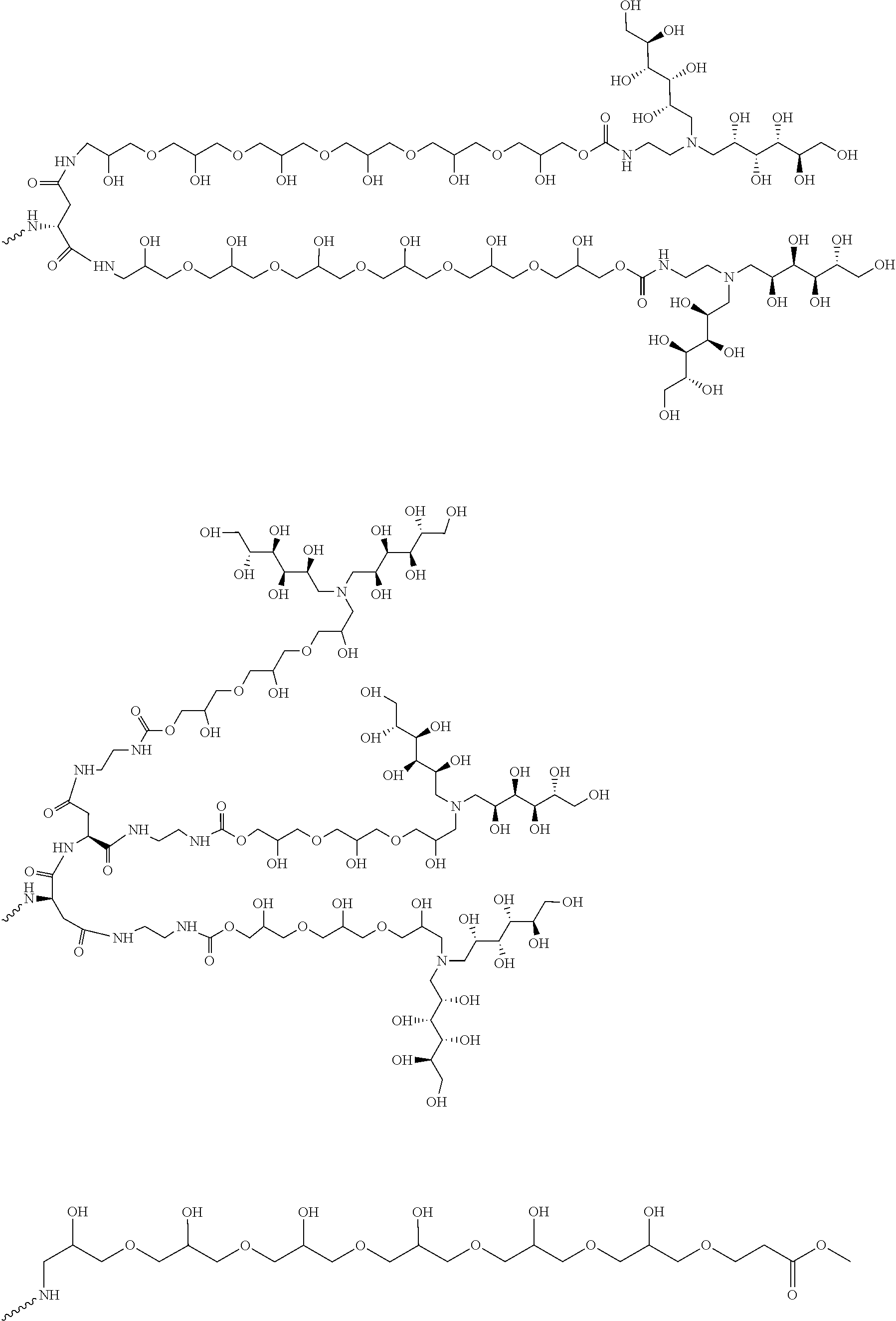

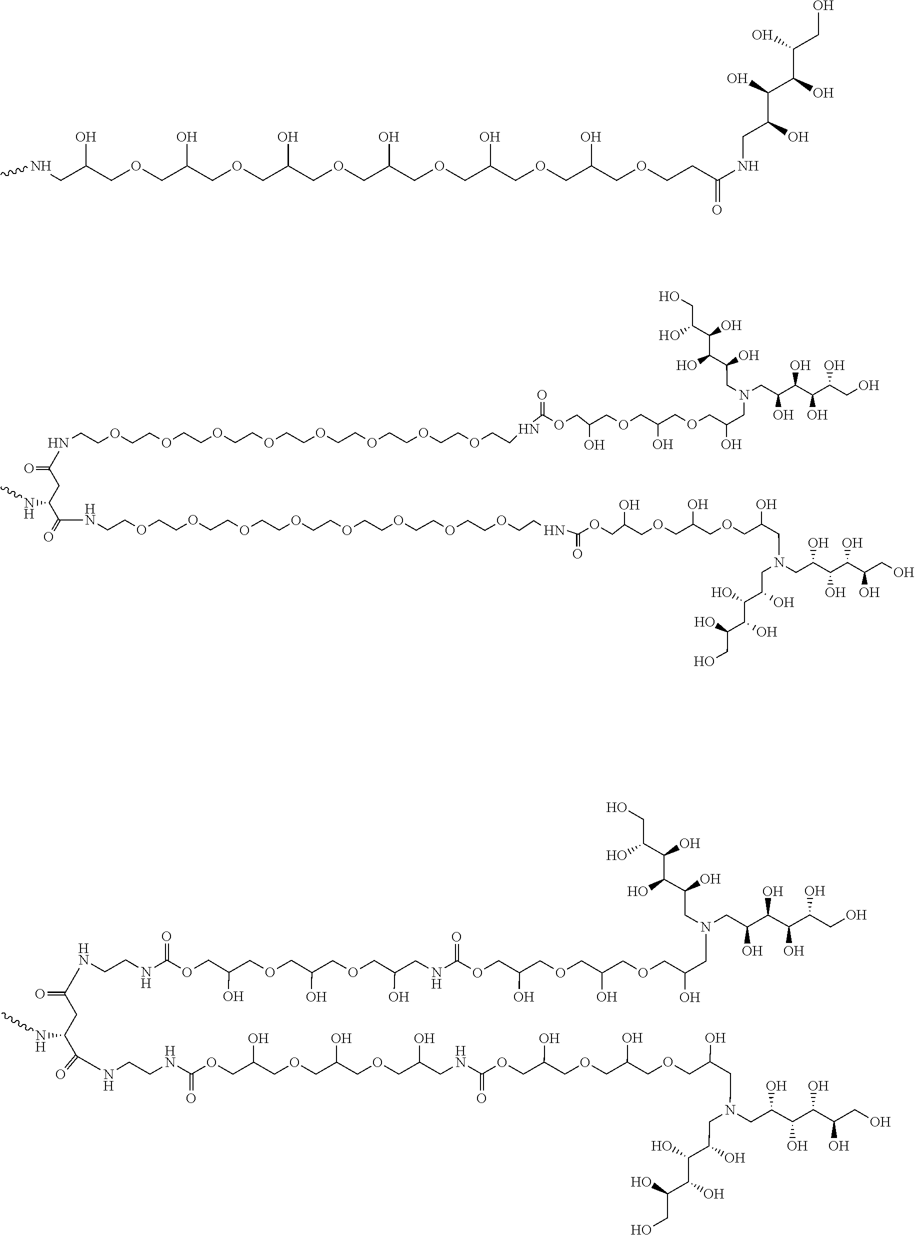

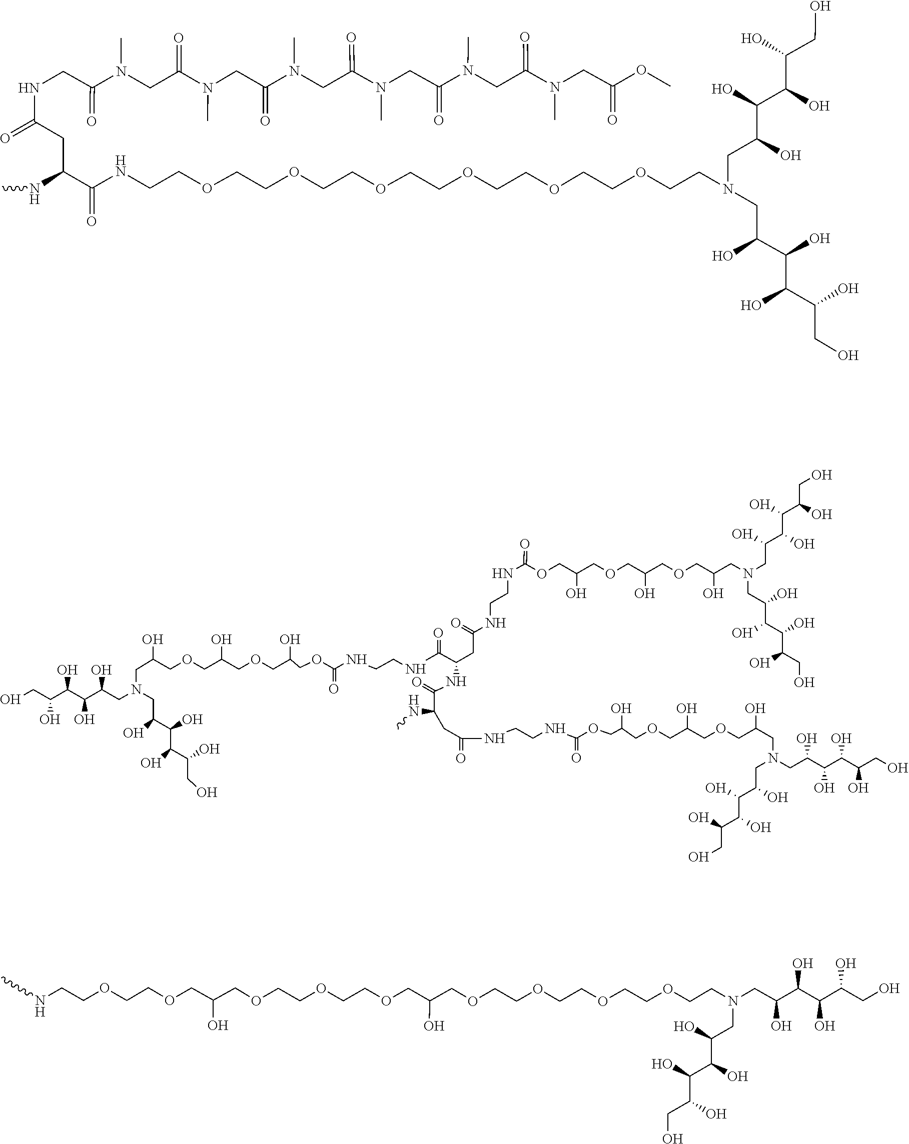

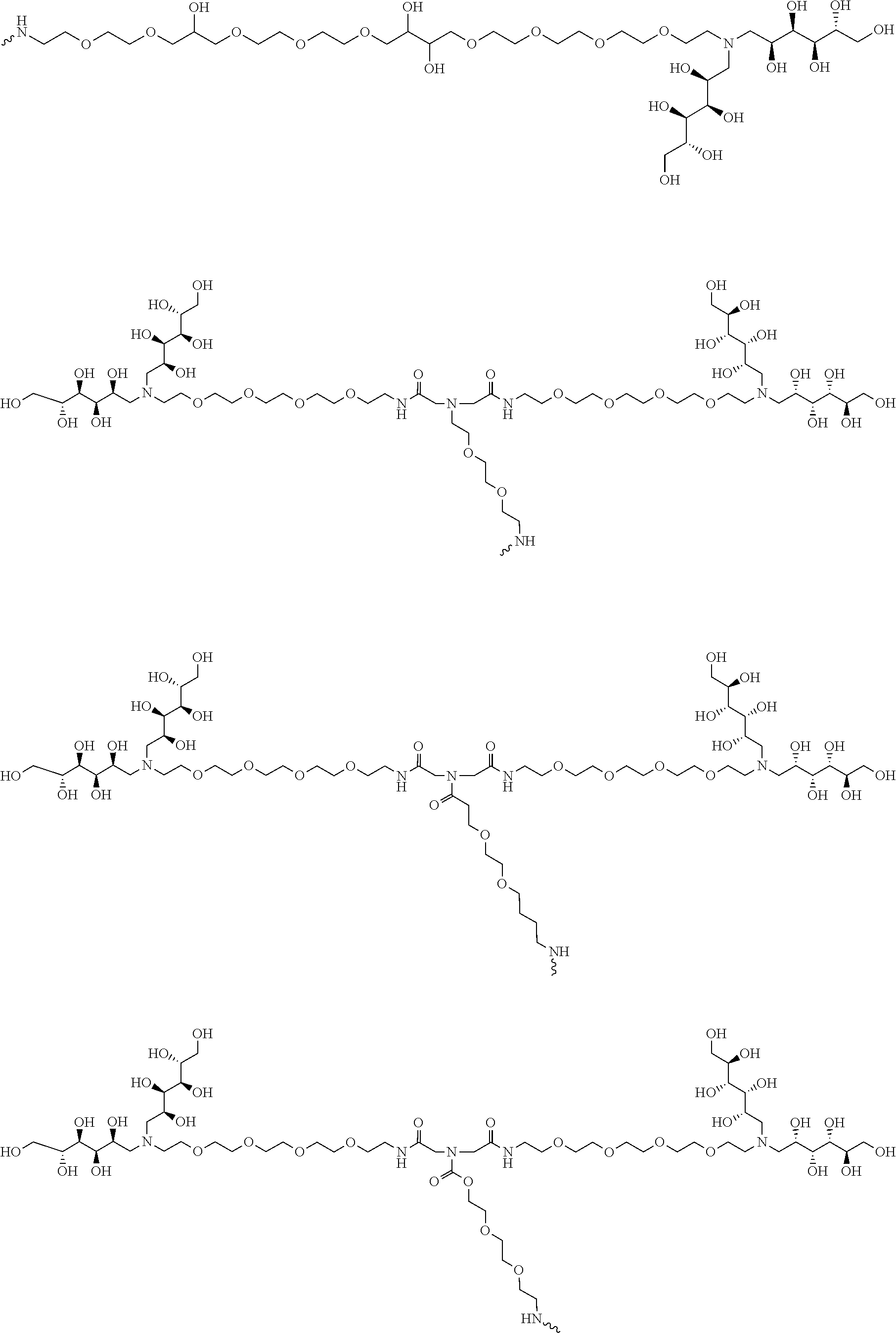

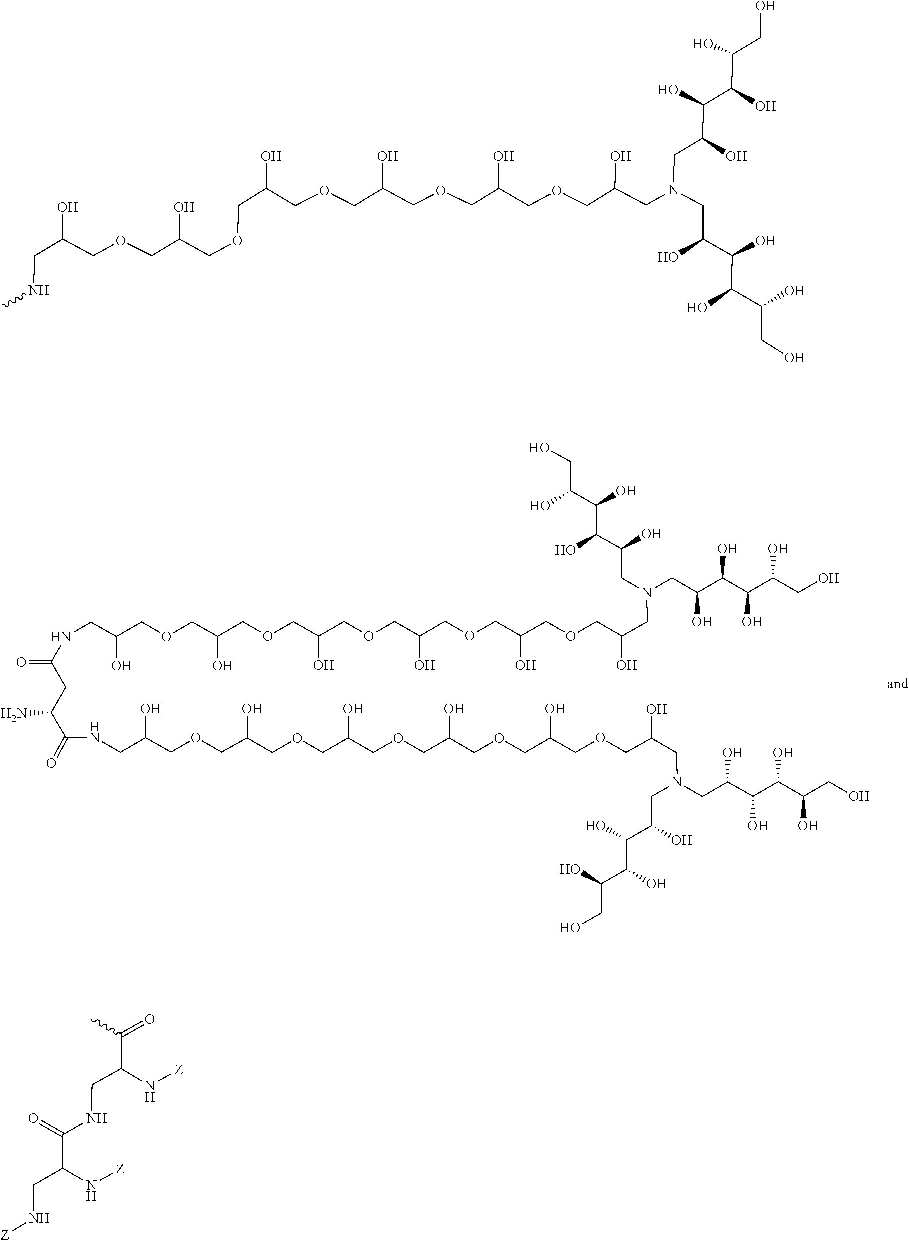

wherein each Z is attached at * and is individually selected from:

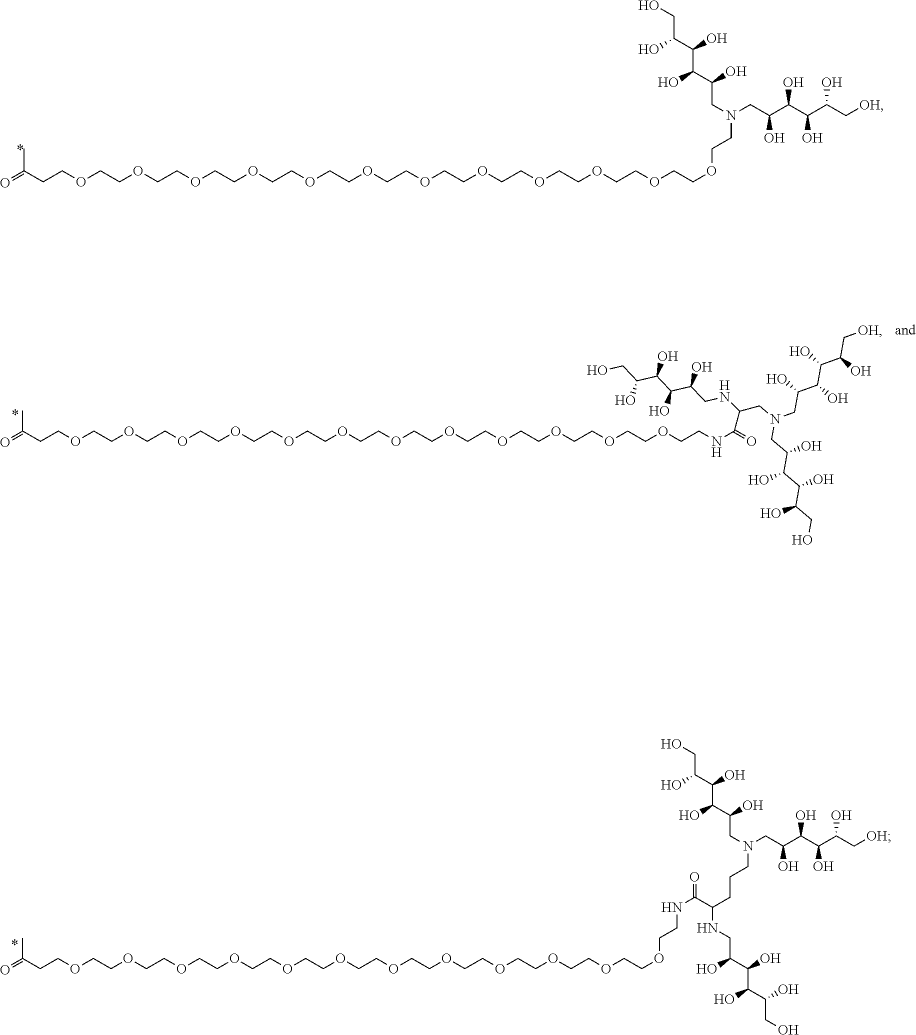 [SMILES: CC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO ; CC(=O)CCOCCOCCOCCOCCOCCOCCOCCOCCOCCOCCOCCOCCNC(=O)C(CN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO ; CC(=O)CCOCCOCCOCCOCCOCCOCCOCCOCCOCCOCCOCCOCCNC(=O)C(CCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

wherein each [symbol] is an attachment to site β or to site R^{b}, or to the enzyme-cleavable group.

Embodiment 77. The linker compound of any one of Embodiments 1 to 75, wherein the polar group is selected from the following:

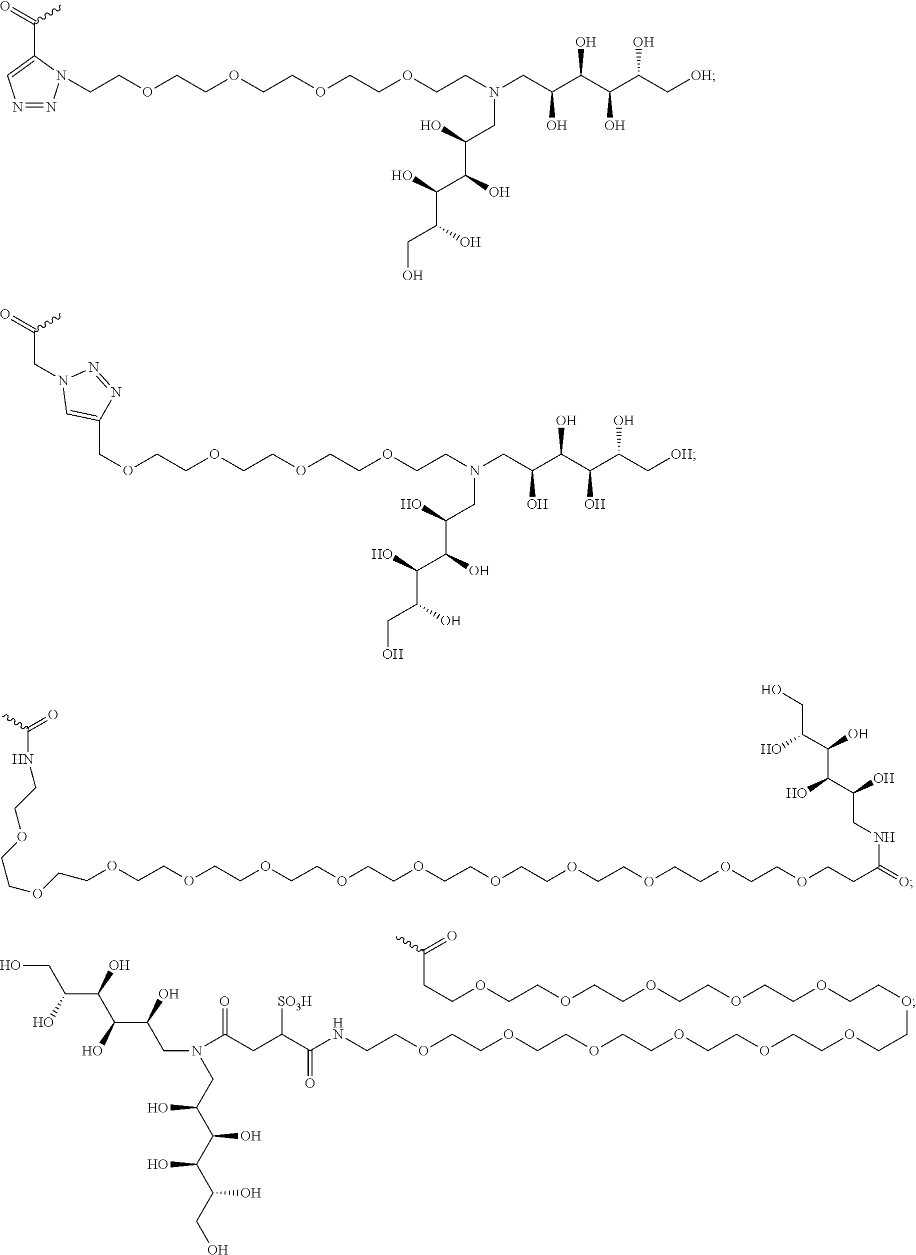

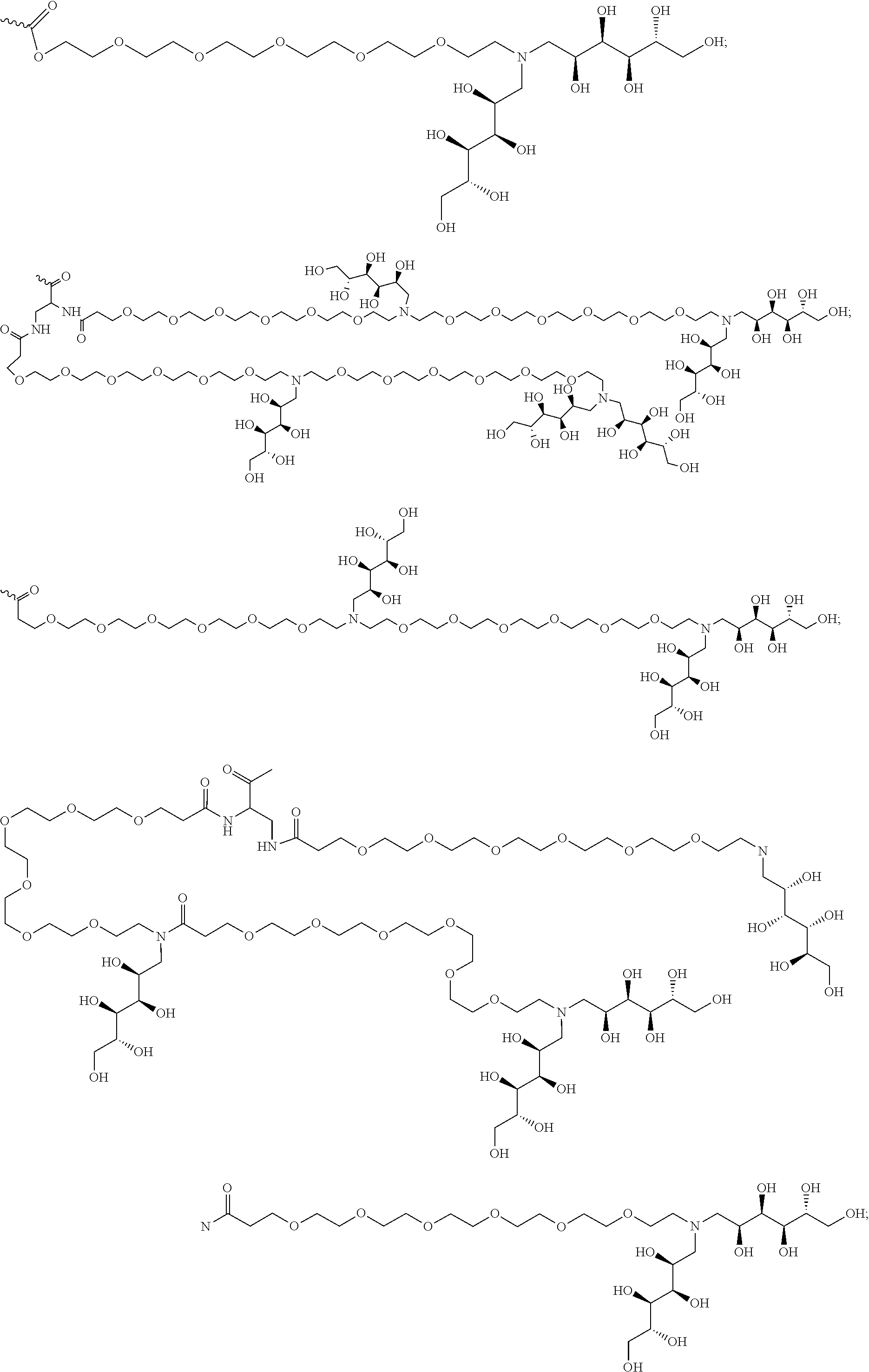

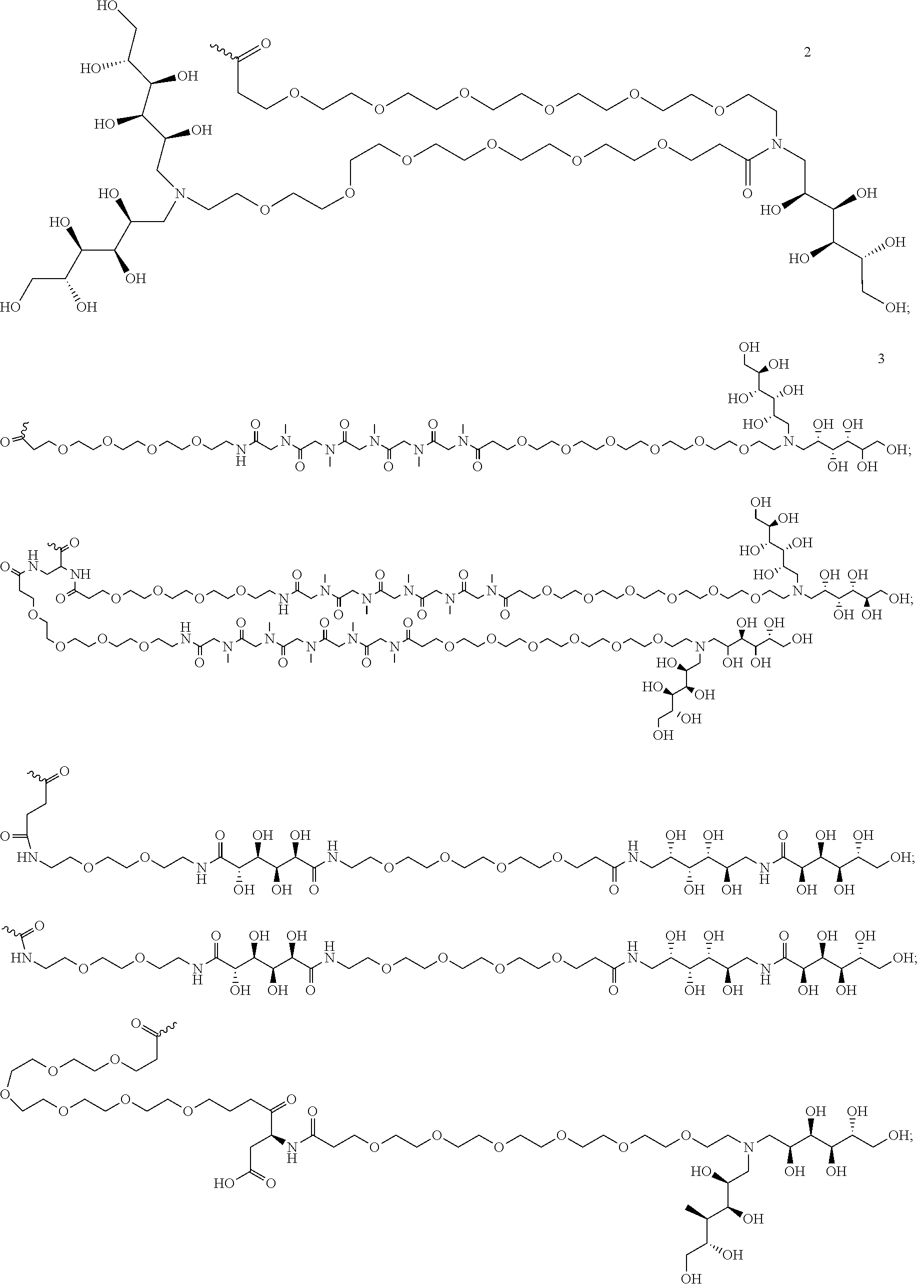

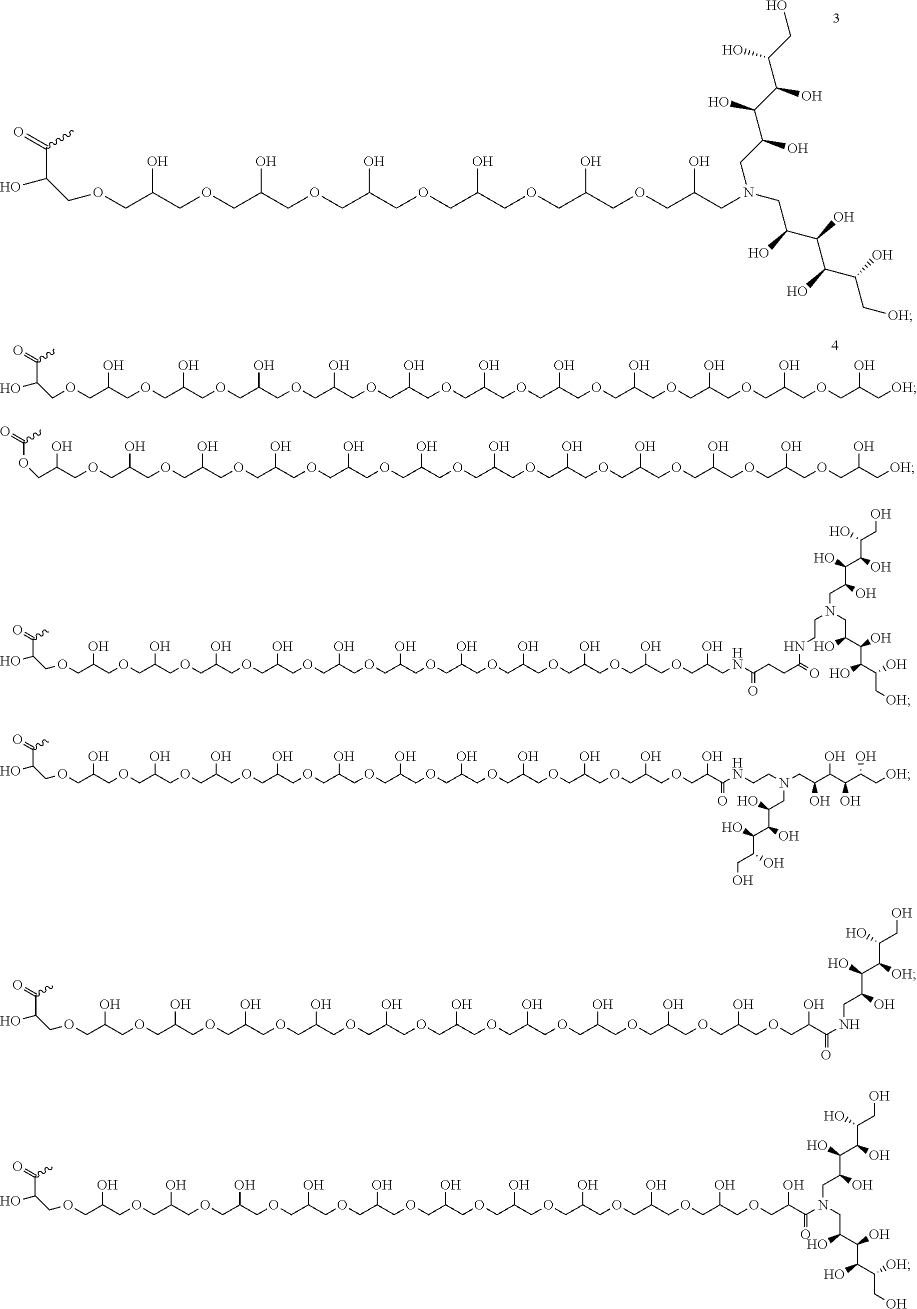

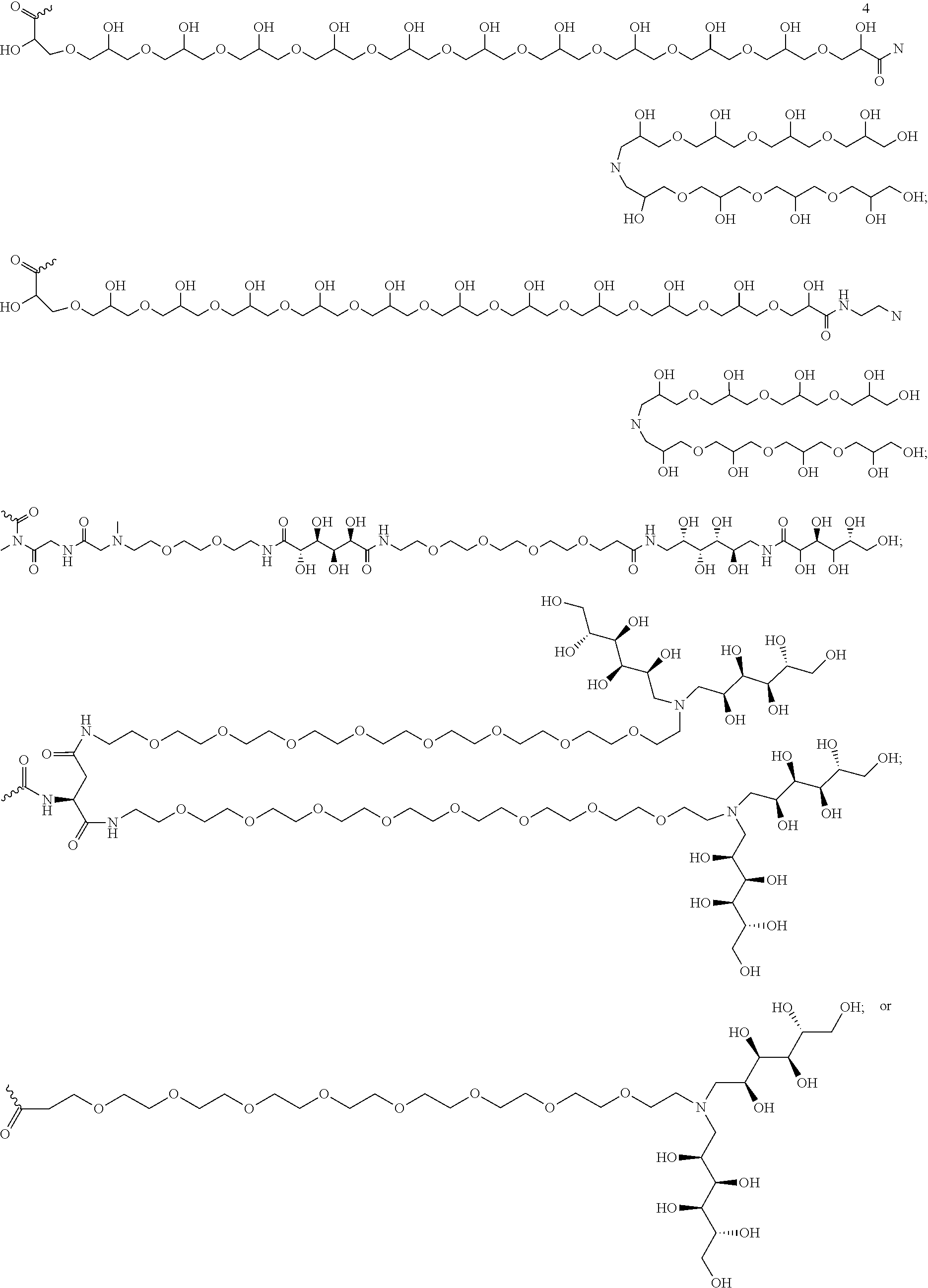

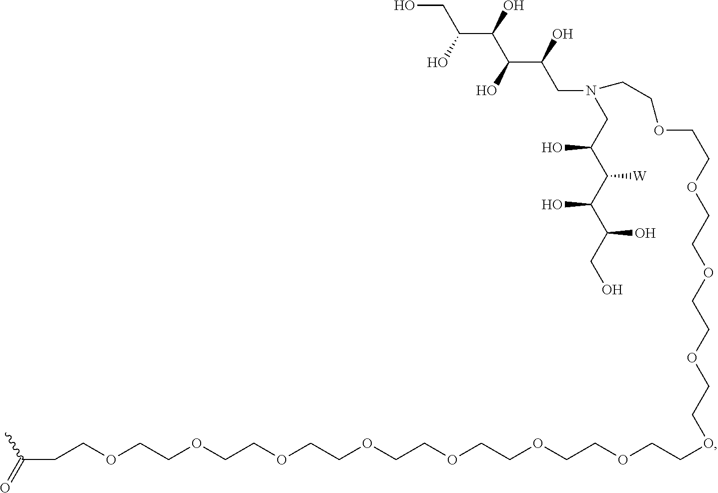

or a stereoisomer thereof, wherein each [symbol] indicates an attachment site of the amino acid unit.

Embodiment 78. The linker compound of any one of Embodiments 1 to 77, wherein the polar group comprises at least one carboxyl unit having the following formula:

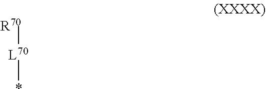

or a stereoisomer or salt thereof, wherein:
(a)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
   R⁷⁰ is ˜NR⁷¹(R⁷²—R⁷³), wherein R⁷¹ is selected from H, C₁-C₁₂ alkyl, substituted C₁-C₁₂ alkyl, or polyethylene glycol (optionally having 1 to 12 ethylene glycol subunits), R⁷² is a bond or is selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and R⁷³ is a carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(b)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
   R⁷⁰ is ˜NR⁷¹(R⁷⁵ (R⁷³)₂), wherein R⁷¹ is selected from H, C₁-C₁₂ alkyl, substituted C₁-C₁₂ alkyl, or polyethylene glycol (optionally having 1 to 12 ethylene glycol subunits), R⁷⁵ is a branched optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl and each R⁷³ is independently carboxyl or polycarboxyl, wherein polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide; or
(c)
   L⁷⁰ is selected from C₁-C₈ alkylene, C₁-C₈ alkylene-C(O)—, —C(O)—C₁-C₈ alkylene-, and —C(O)—C₁-C₈ alkylene-C(O)—, and * is an attachment site to R^{b}, to the enzyme-cleavable group, or to a remainder of the polar group;
   R⁷⁰ is —N(R⁷⁴—R⁷³)(R⁷²R⁷³), wherein R⁷² and R⁷⁴ are each independently selected from optionally substituted C₁-C₃ alkylene, optionally substituted ether, optionally substituted thioether, optionally substituted ketone, optionally substituted amide, polyethylene glycol (optionally having 1 to 12 ethylene glycol subunits), optionally substituted carbocycle, optionally substituted aryl or optionally substituted heteroaryl, and each R⁷³ is independently carboxyl or polycarboxyl, wherein the polycarboxyl comprises 1 to 10, or 1 to 6, or 1 to 4 carboxyl groups, wherein the carboxyl groups are interconnected by alkyl, alkylene, substituted alkyl, substituted alkylene, heteroalkyl, heteroalkylene, amino and/or amide.

Embodiment 79. The linker compound of any one of Embodiments 1 to 78, the polar group includes the polymer unit and the sugar unit.

Embodiment 80. The linker compound of any one of Embodiments 1 to 78, the polar group includes at least two polymer units.

Embodiment 81. The linker compound of any one of Embodiments 1 to 78, the polar group includes the polymer unit and the carboxyl unit.

Embodiment 82. The linker compound of any one of Embodiments 1 to 78, comprising at least two polar groups.

Embodiment 83. The linker compound of any one of Embodiments 1 to 78, the polar group includes the polymer unit, the sugar unit and the carboxyl unit.

Embodiment 84. The linker compound of any one of Embodiments 1 to 78, the polar group includes at least two polymer units, at least one sugar unit and at least one carboxyl unit.

Embodiment 85. The linker compound of any one of Embodiments 9 to 78, wherein the enzyme-cleavable group comprises at least two amino acids.

Embodiment 86. The linker compound of any one of Embodiments 9 to 85, comprising at least one of the polar groups attached to the enzyme-cleavable group.

Embodiment 87. The linker compound of any one of Embodiments 1-86, having one of the following structures:

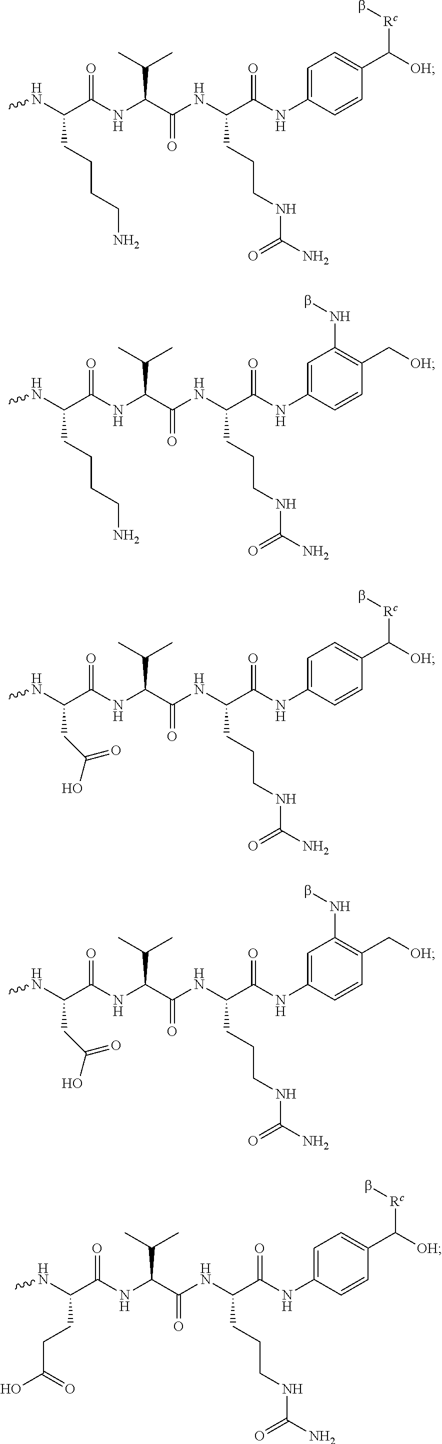

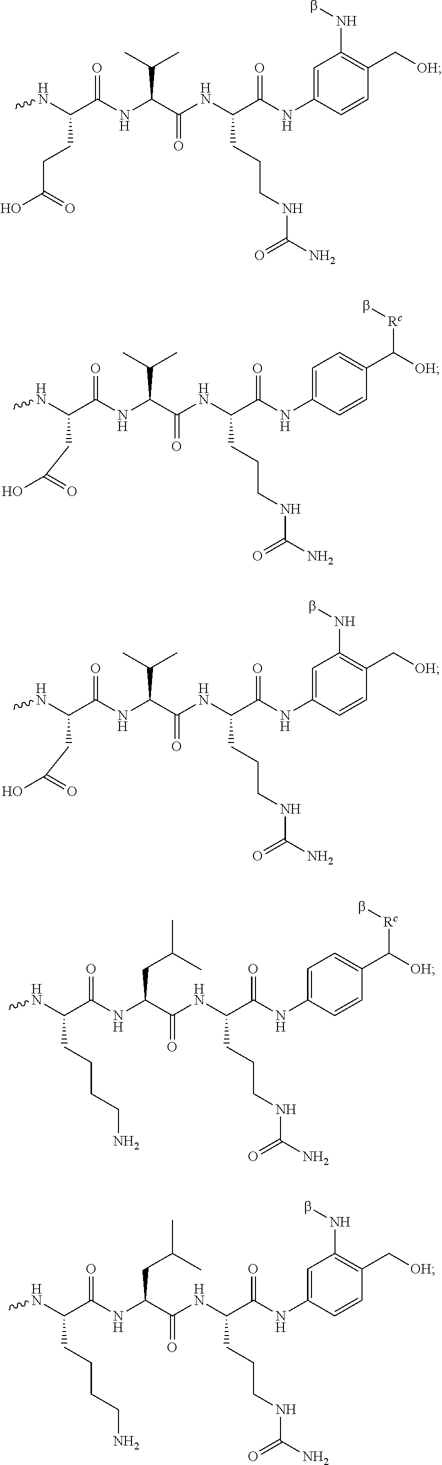

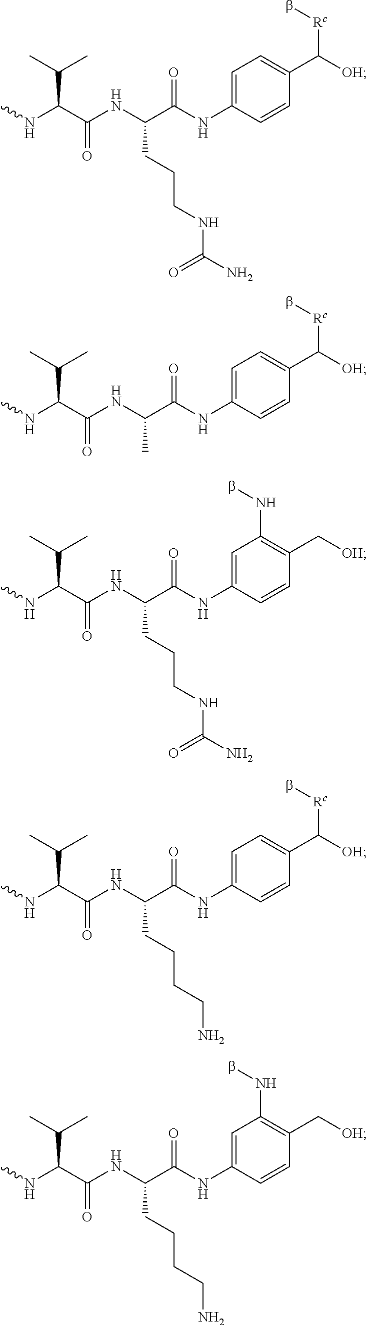

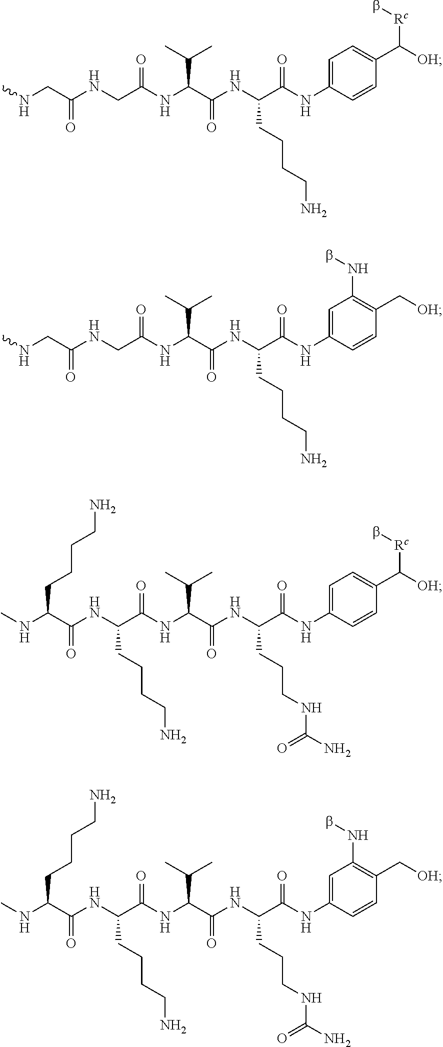

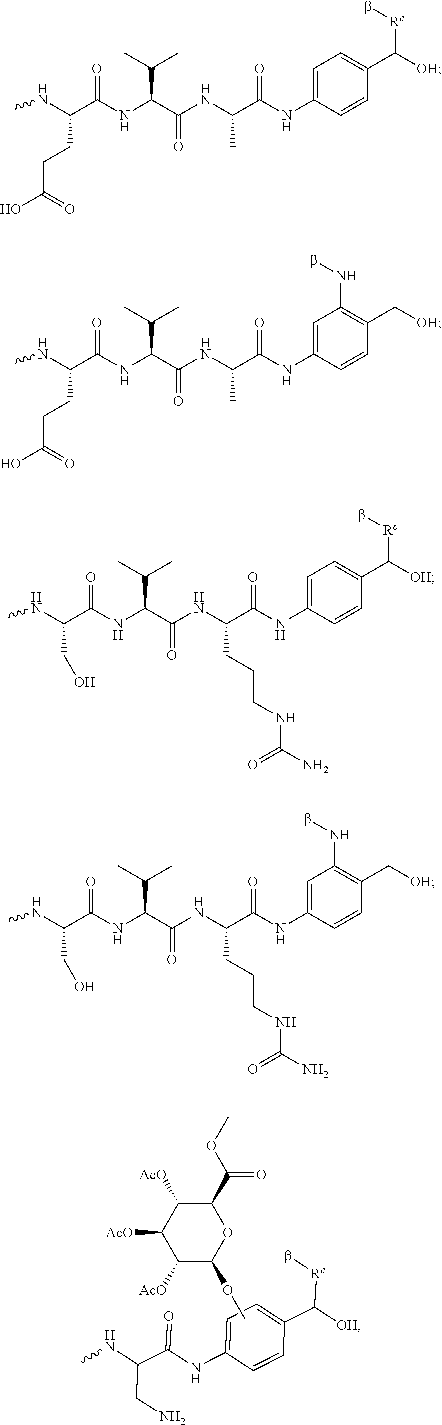

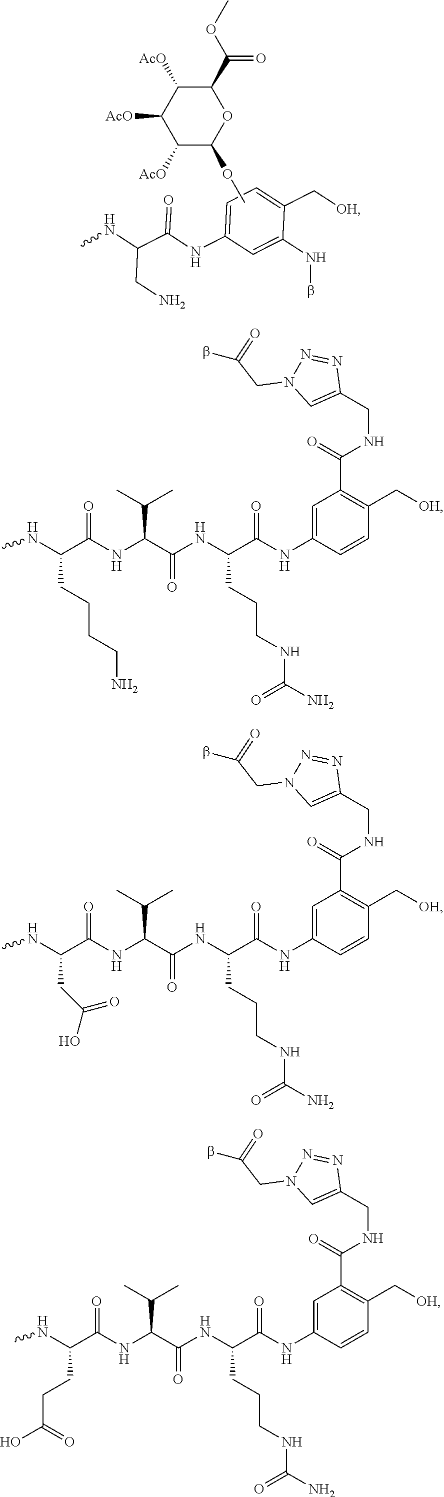

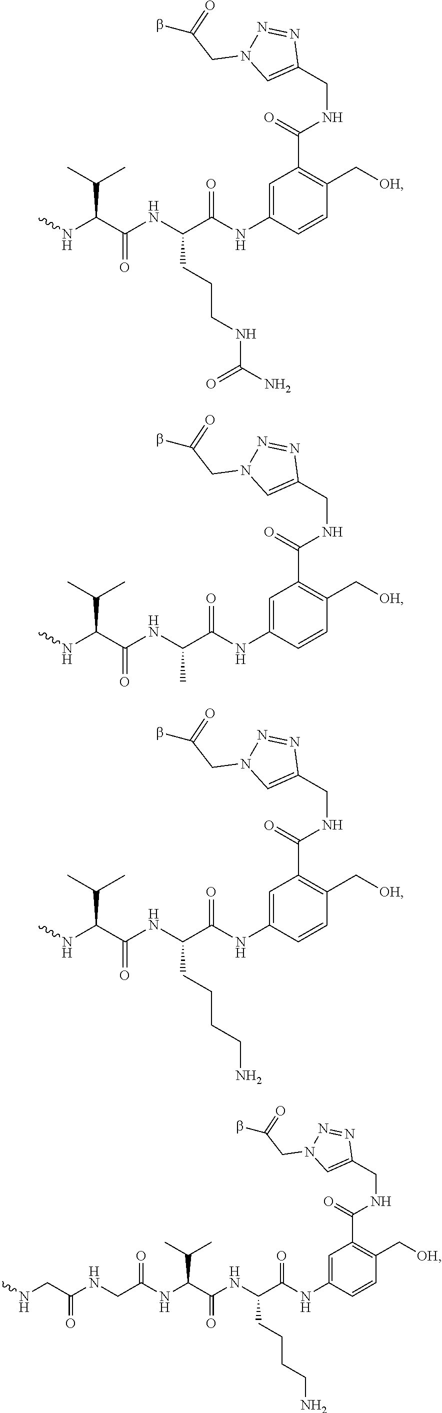

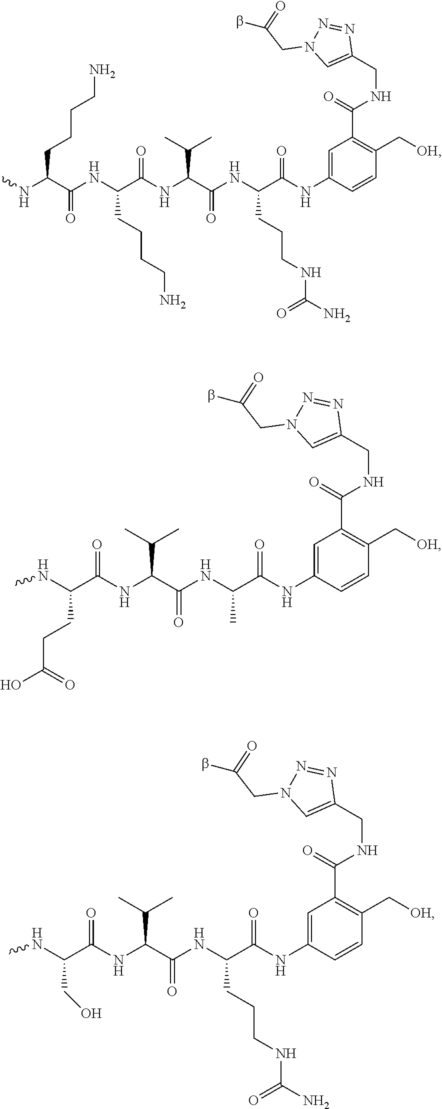

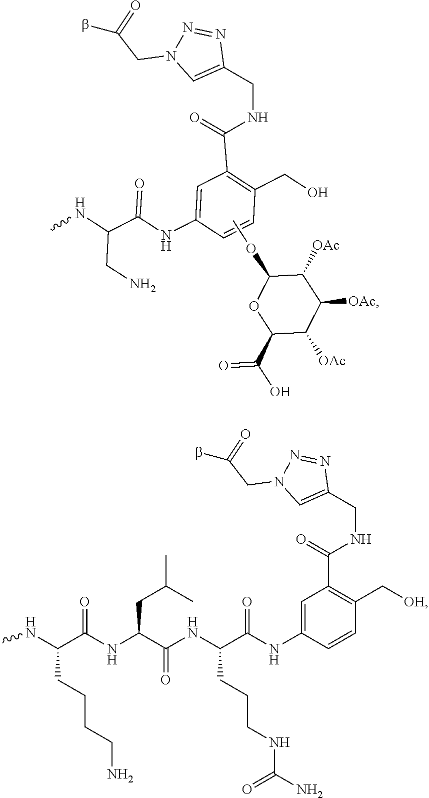

wherein
R^{c} is a bond or C₁₋₆ alkylene;
the wavy line on the amino group indicates an attachment site for a stretcher group or, prior to attachment to the stretcher group, indicates H;
β—is the attachment site to the at least one polar group; and
the benzylic H on the benzylic OH is optionally replaced with a bond to at least one of the the drug unit or to a linking group attached to at least one of the drug units.

Embodiment 88. The linker compound of any one of Embodiments 1-86, comprising one of the following structures:

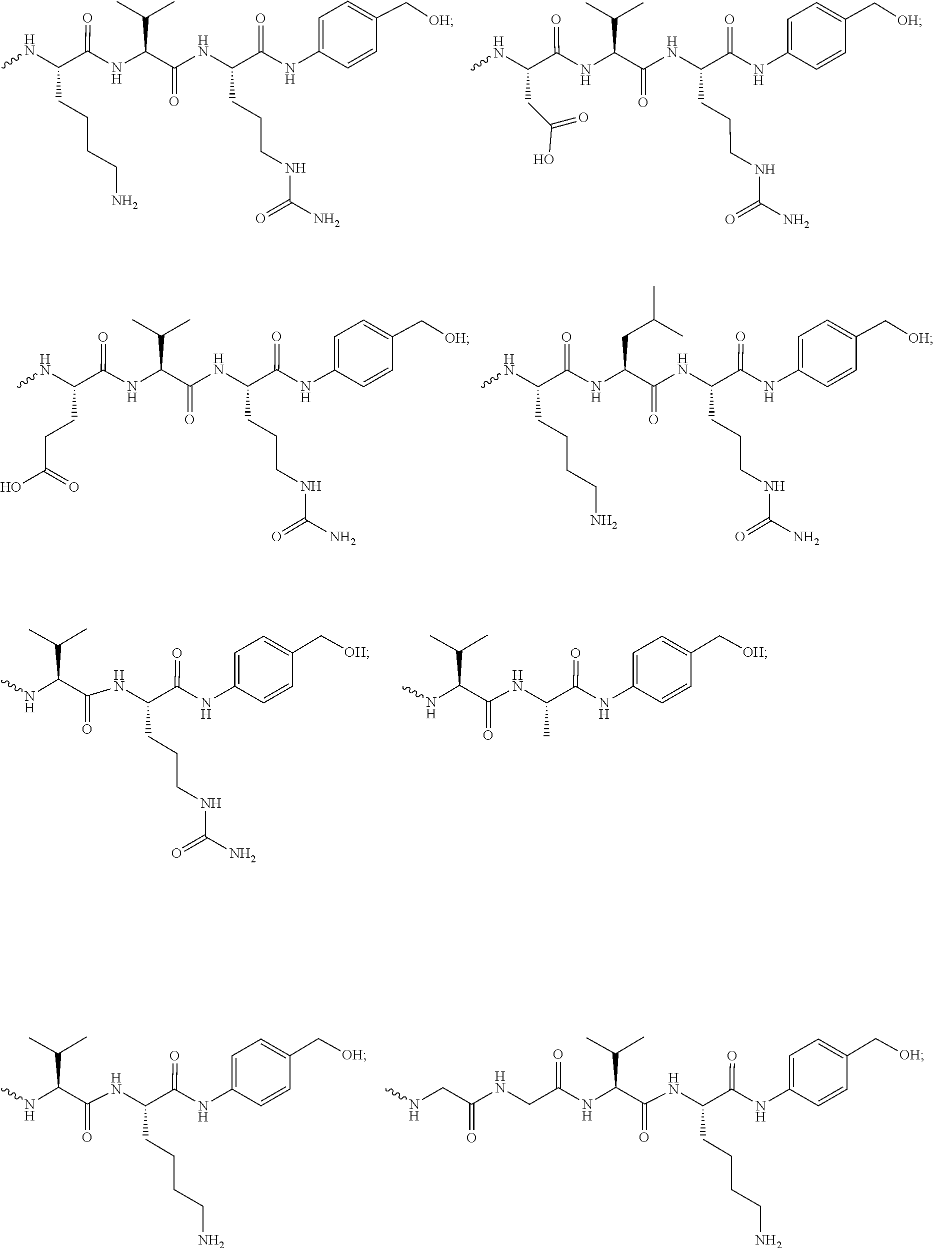

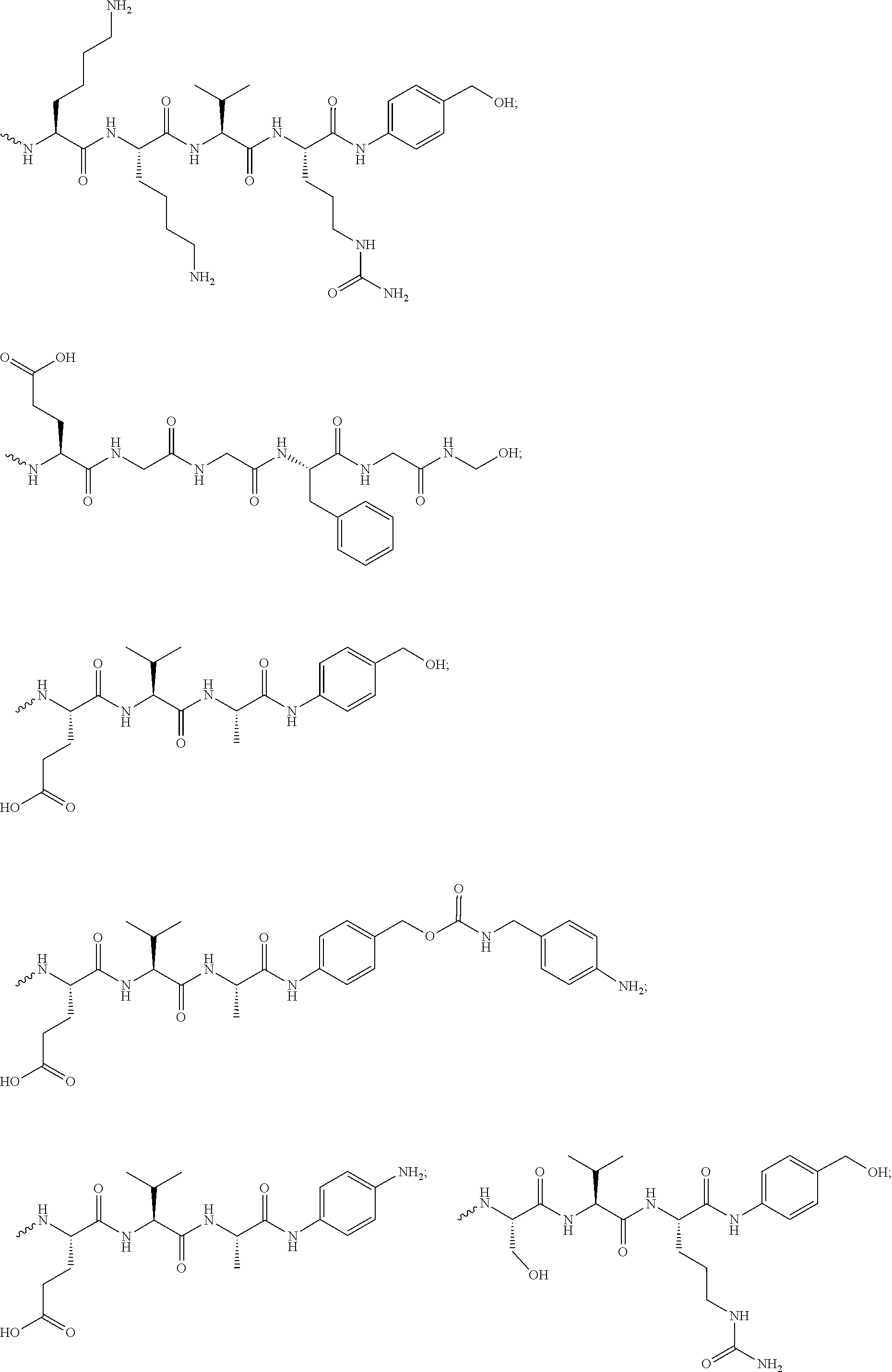

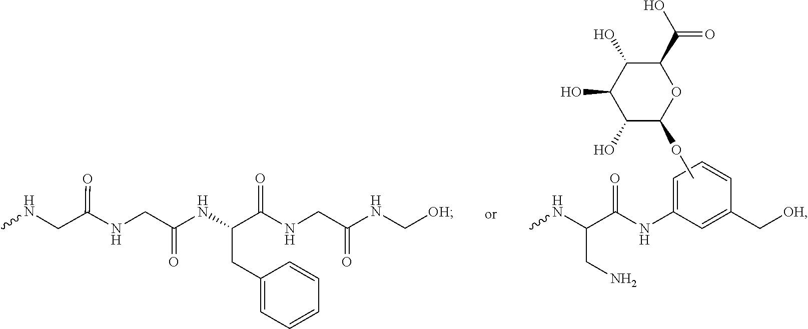

or a stereoisomer thereof, wherein the polar group is attached to an amino acid subunit of the amino acid unit, the H of a hydroxyl or amino group of the para-aminobenzyl group or the H of a hydroxyl of the glycine residue of a GGFG peptide is optionally replaced with a bond to at least one of the drug units, or to a linking group attached to the at least one of the drug units, the wavy line on the amino group indicates an attachment site for the stretcher group or the amino acid unit or, prior to attachment, indicates H.

Embodiment 89. The linker compound of any one of Embodiments 1-88, comprising a formula selected from the following:

˜[SU-aa]-L2≈,

˜[aa₁(POLY)-aa]-L2≈, or

˜[CU-aa]-L2≈
wherein the square brackets indicate the amino acid unit, each aa is an optional subunit of the amino acid unit, L2 is the linker unit, each wavy line (˜) indicates an attachment site for a stretcher group; aa₁(POLY) is the polymer unit attached to an amino acid subunit of the amino acid unit, SU is the sugar unit attached to a subunit of the amino acid unit or to the linker unit, and CU is the carboxyl unit attached to a subunit of the amino acid unit or to the linker unit; and the double wavy (≈) line indicates an attachment site for at least one of the drug units, wherein aa and aa₁ are independently selected from alpha, beta and gamma amino acids and derivatives thereof.

Embodiment 90. The linker compound of any one of Embodiments 1-88, comprising a formula selected from the following:

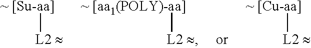

wherein the square brackets indicate the amino acid unit, each aa is an amino acid subunit of the amino acid unit, L2 is the linker Subunit attached to a side chain of aa, the wavy line (˜) indicates an attachment site for a stretcher group; aa₁(POLY) is the polymer unit attached to aa, SU is the sugar unit attached to aa, CU is the carboxyl unit attached to aa, and the double wavy (≈) line indicates an attachment site for at least one of the drug units; wherein aa and aa₁ are independently selected from alpha, beta and gamma amino acids and derivatives thereof.

Embodiment 91. The linker compound of any one of Embodiments 1-88, comprising a formula selected from the following:

˜[SU-aa-SU]-L2≈,

˜[aa₁(POLY)-aa-aa₂(POLY)]-L2≈, or

˜[CU-aa-CU]-L2≈

wherein the square brackets indicate the amino acid unit, aa is an optional subunit of the amino acid unit, L2 is the linker unit, the wavy line (˜) indicates an attachment site for a stretcher group; each of aa₁(POLY) and aa₂(POLY) is the polymer unit attached to aa or to the other Polymerunit; each SU is the sugar unit attached to aa or the other sugar unit, each CU is the carboxyl unit attached to aa or to the other carboxyl unit, and the double wavy (≈) line indicates an attachment site for at least one of the drug units; wherein aa, aa₁ and aa₂ are independently selected from alpha, beta and gamma amino acids and derivatives thereof.

Embodiment 92. The linker compound of any one of Embodiments 1-88, comprising a formula selected from the following:

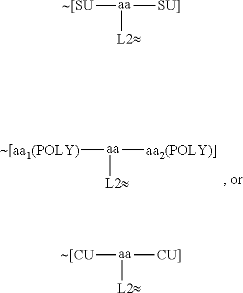

wherein the square brackets indicate the amino acid unit, aa is an amino acid subunit of the amino acid unit, L2 is the linker unit attached to a side chain of aa, each wavy line (˜) indicates an attachment site for a stretcher group; each of aa₁(POLY) and aa₂(POLY) is the polymer unit attached to aa, each SU is the sugar unit attached to aa; each CU is the carboxyl unit attached to aa; and the double wavy (≈) line indicates an attachment site for at least one of the drug units; wherein each of aa, aa₁ and aa₂ is independently selected from alpha, beta and gamma amino acids and derivatives thereof.

Embodiment 93. The linker compound of any one of Embodiments 1-92, wherein the linker unit is a cleavable linker unit.

Embodiment 94. The linker compound of any one of Embodiments 1-92, wherein the enzyme-cleavable group comprises a peptide that is cleavable by an intracellular protease.

Embodiment 95. The linker compound of Embodiment 94, wherein the intracellular protease is Cathepsin B.

Embodiment 96. The linker compound of Embodiment 94, wherein the enzyme-cleavable group comprises a cleavable peptide including a valine-citrulline peptide, a valine-alanine peptide, a valine-lysine peptide, a phenylalanine-lysine peptide, or a glycine-glycine-phenylalanine-glycine peptide.

Embodiment 97. The linker compound of any one of Embodiments 94-96, wherein the cleavable peptide is attached to a para-aminobenzyl alcohol self immolative group (PABA).

Embodiment 98. The linker compound of any one of Embodiments 1-14, comprising one of the following structures:

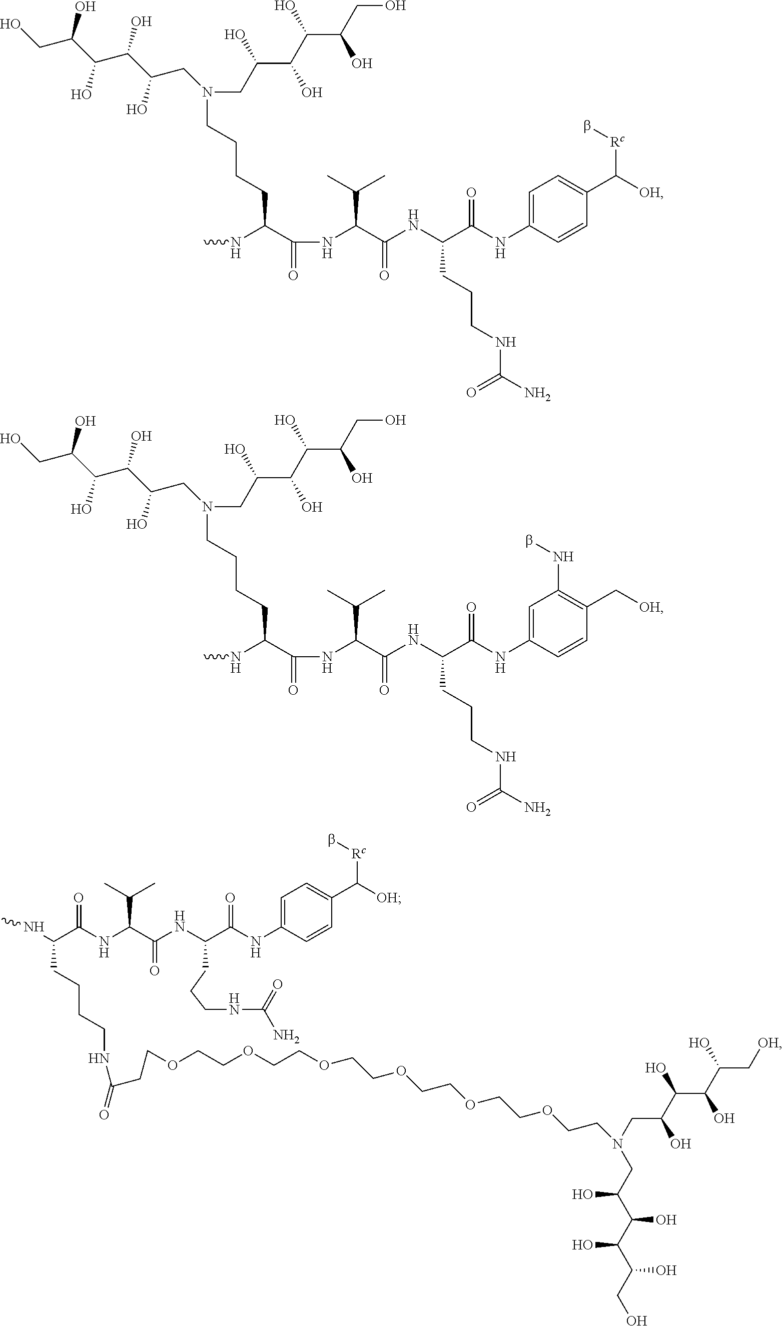

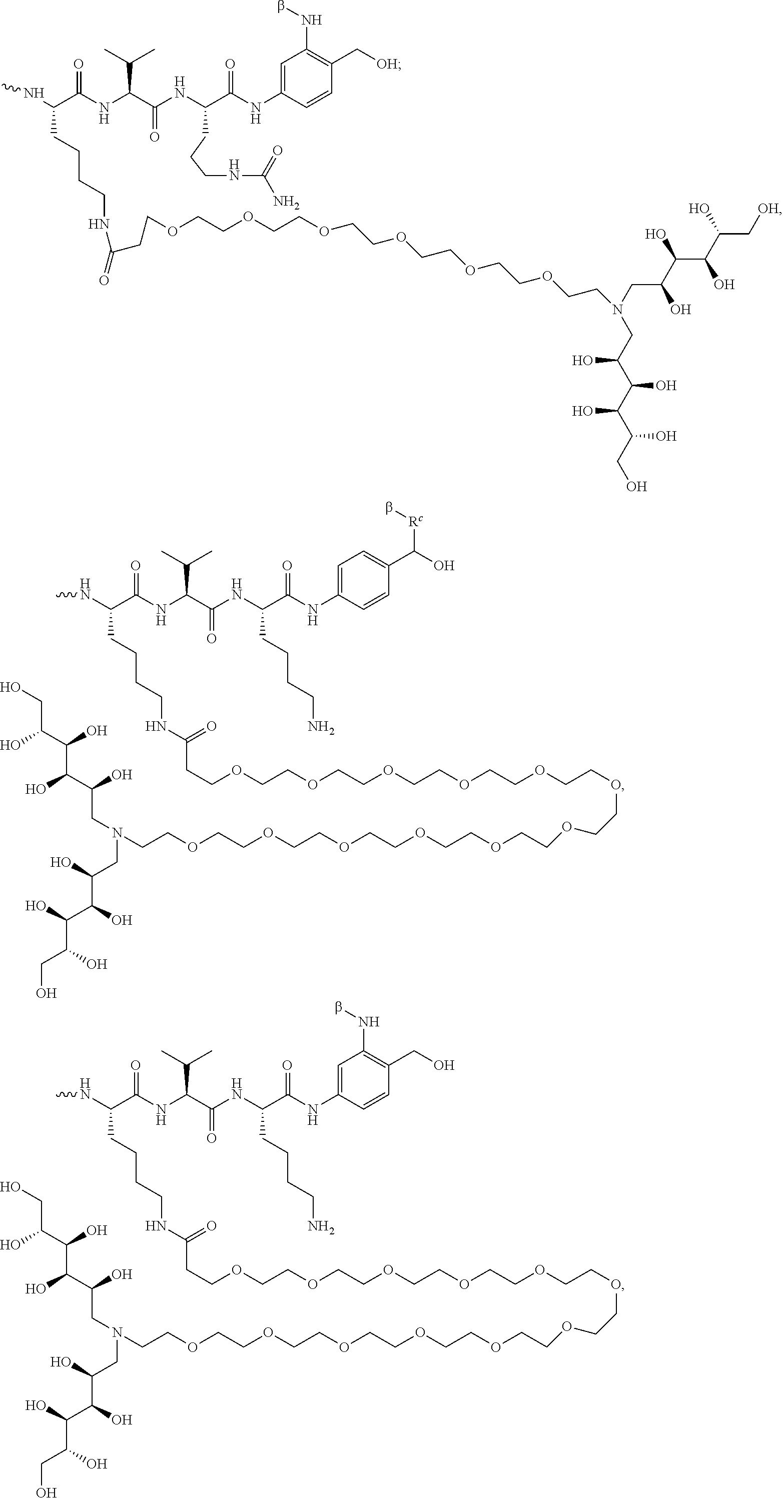

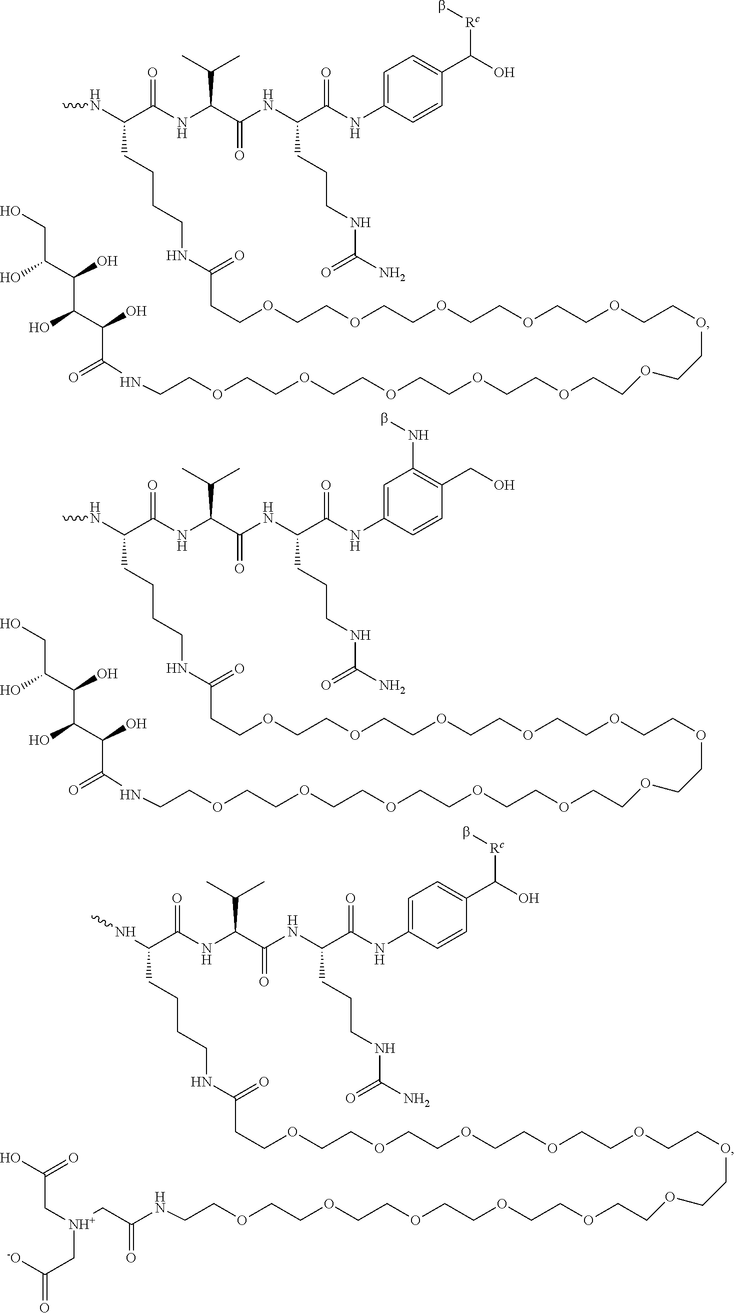

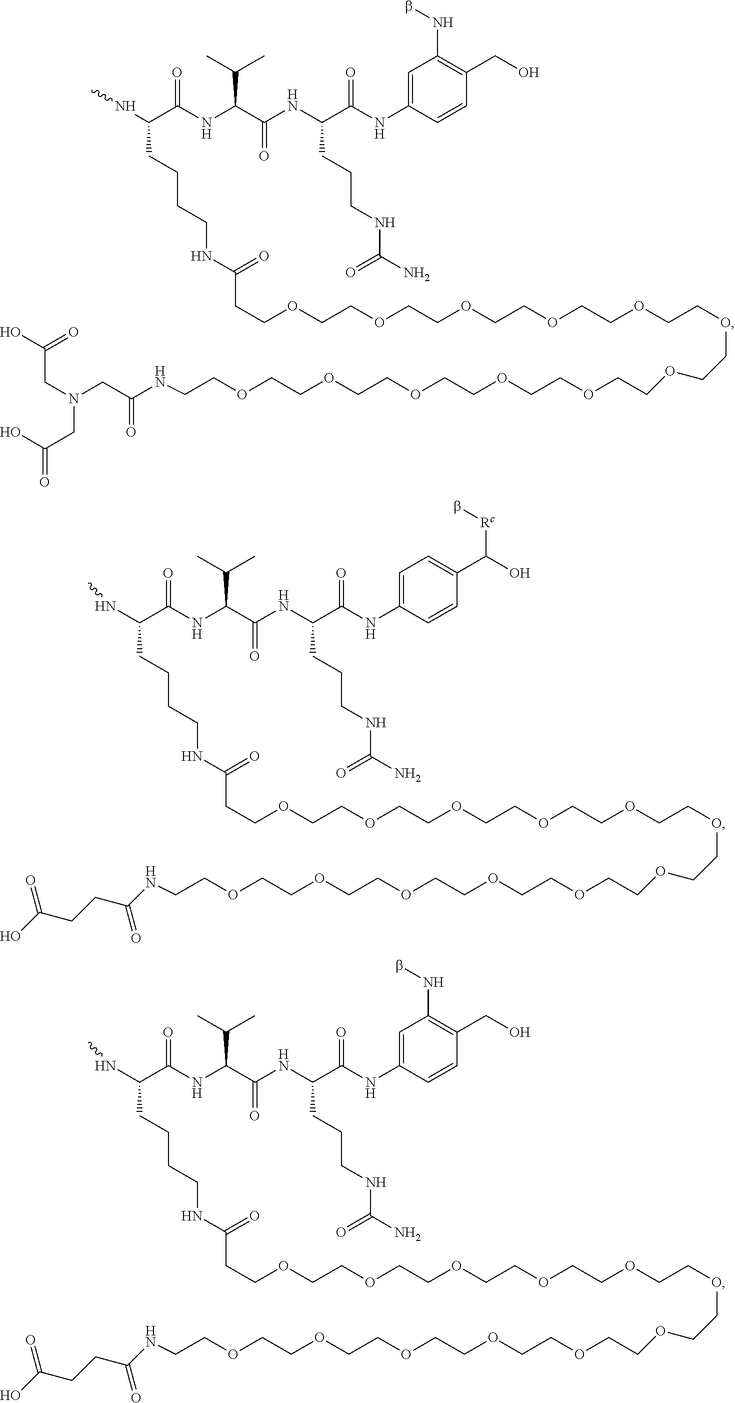

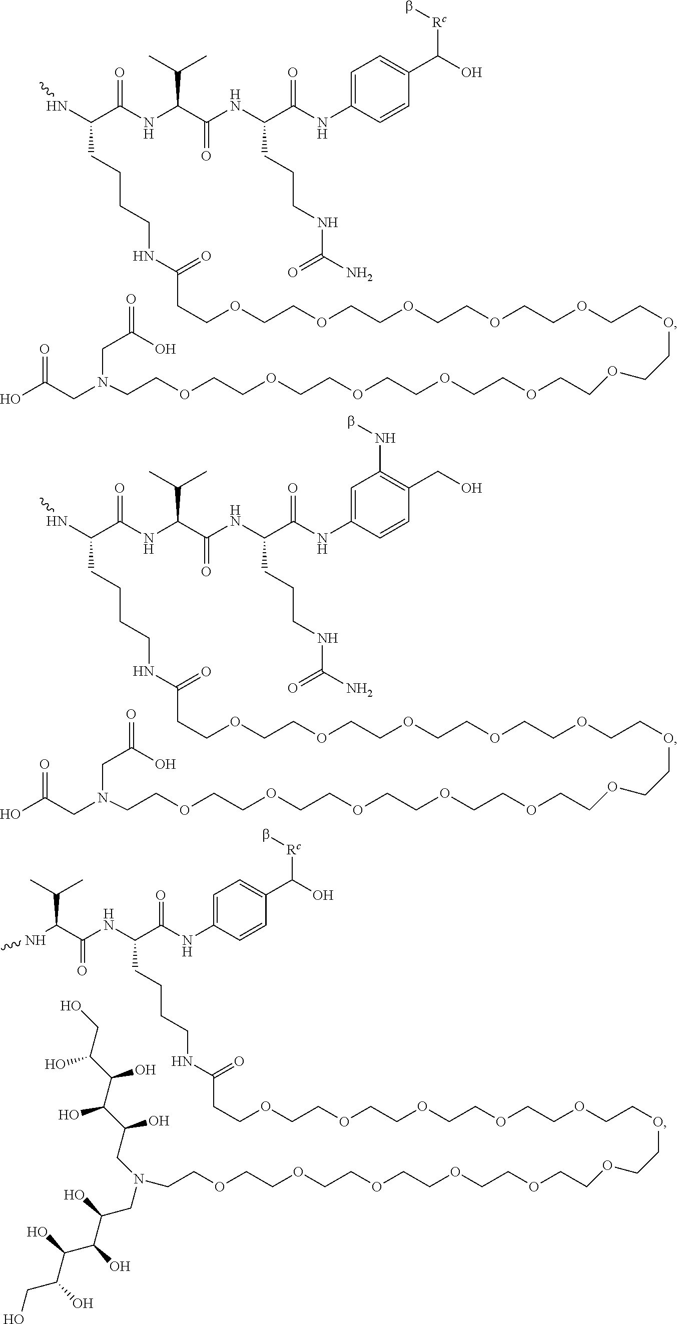

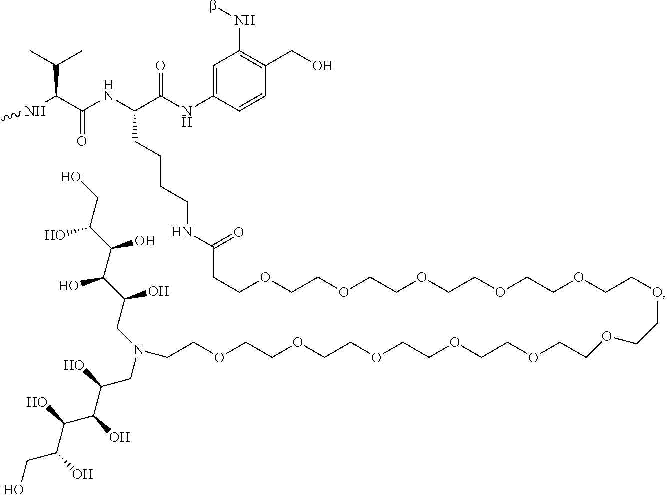

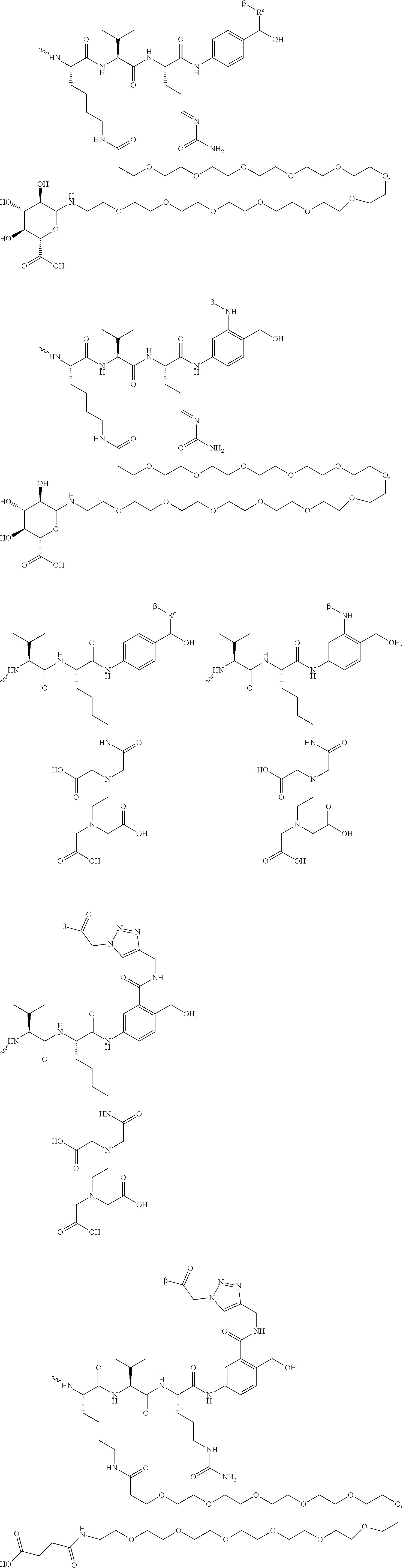

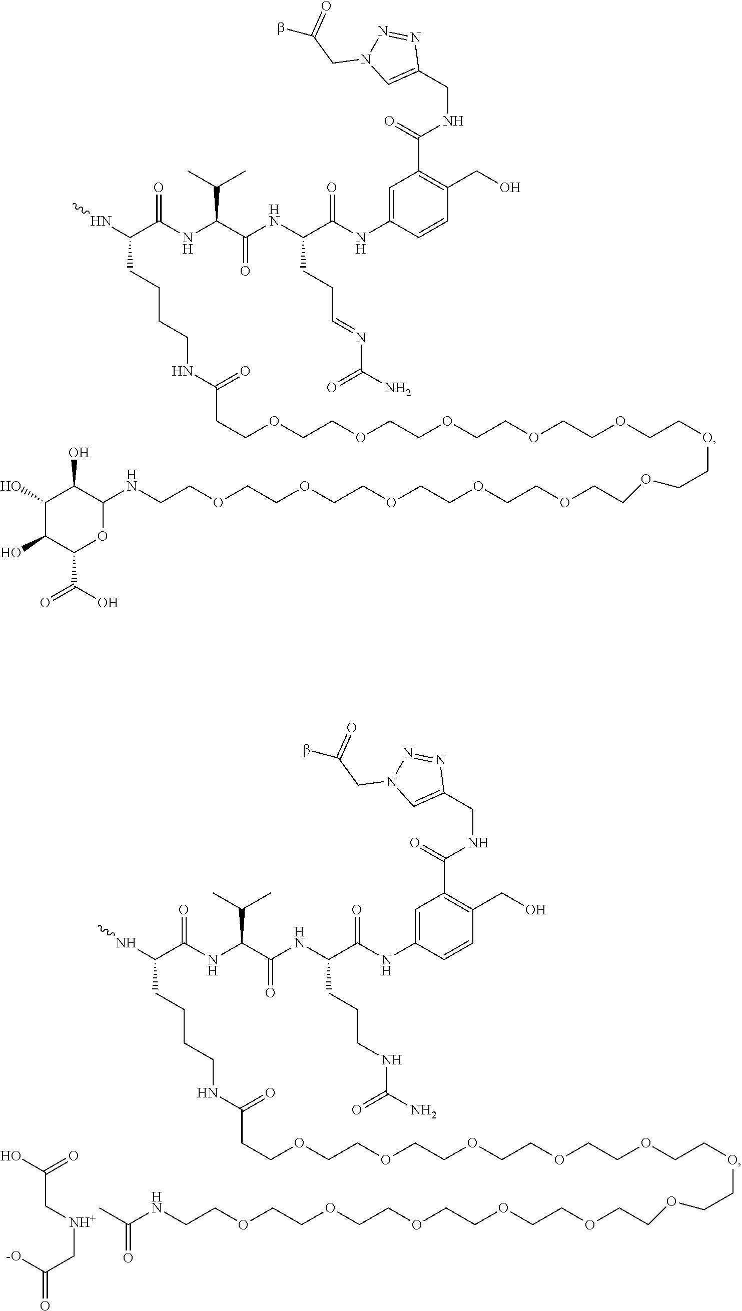

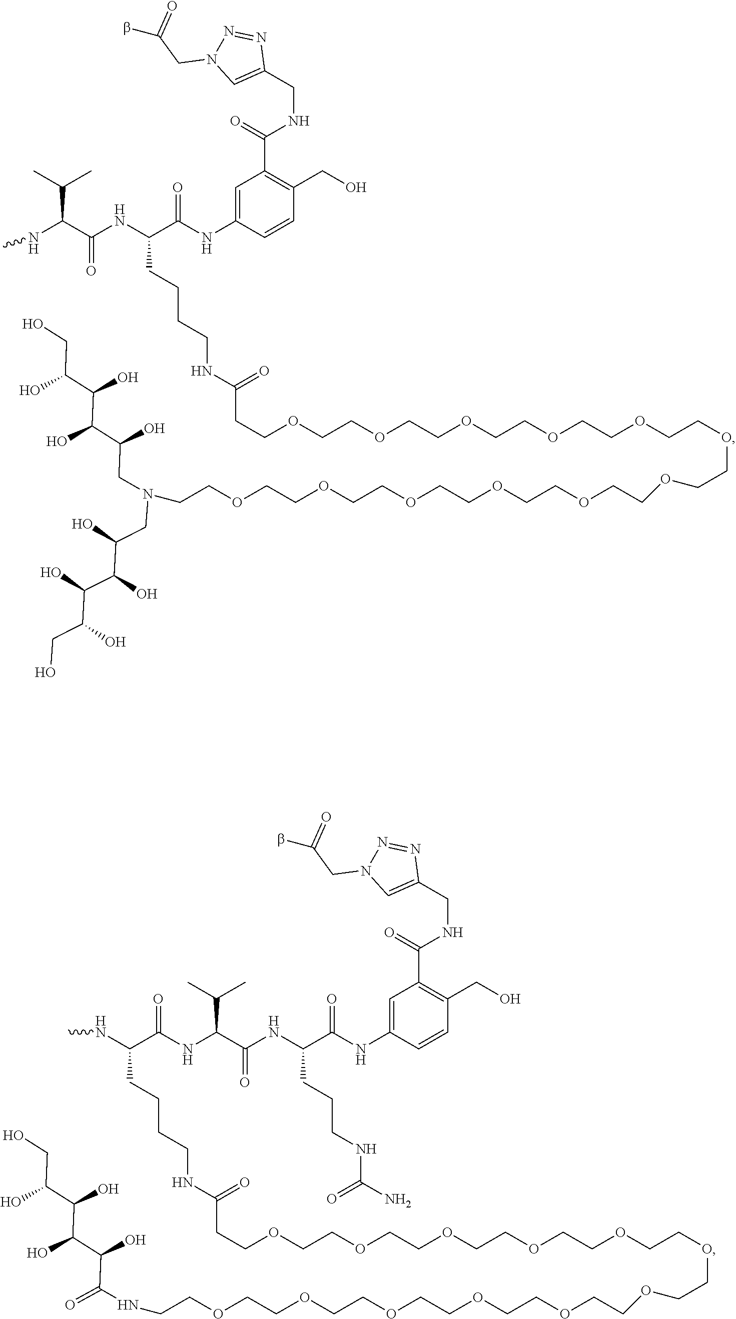

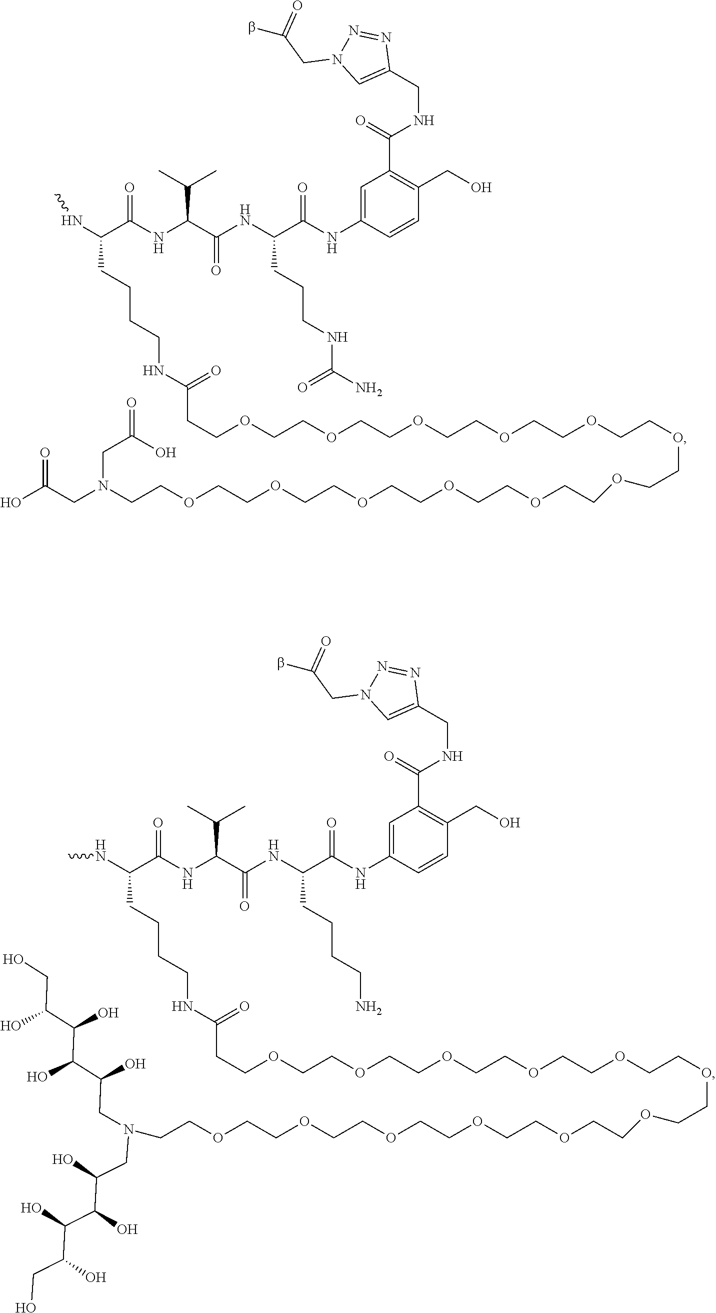

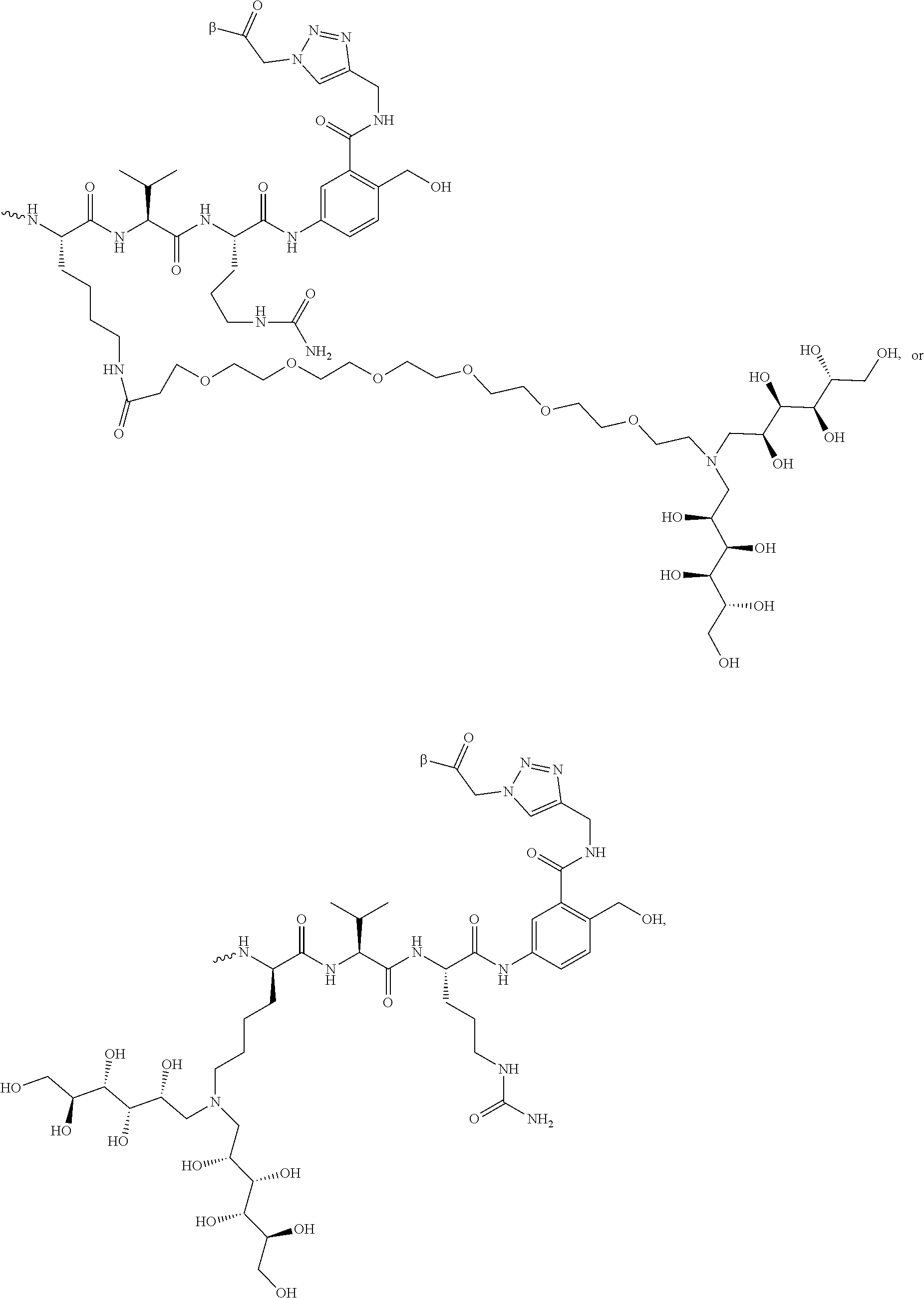

wherein
R^{c} is a bond or C₁₋₆ alkylene;
the wavy line on the amino group indicates an attachment site for the stretcher group or, prior to attachment to the stretcher group, indicates H;
β—is an attachment site to a POLY unit; and
the H on the benzylic OH is optionally replaced with a bond to at least one of the drug units or to the attachment site to at least one of the drug units.

Embodiment 99. The linker compound of any one of Embodiments 1-14, having one of the following structures:

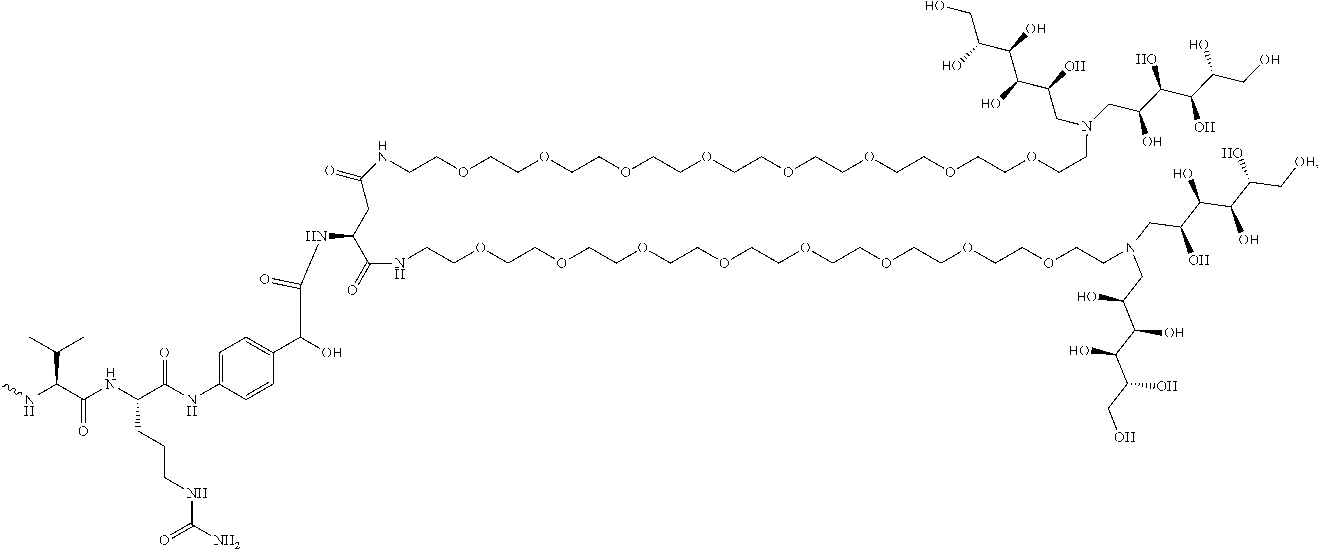

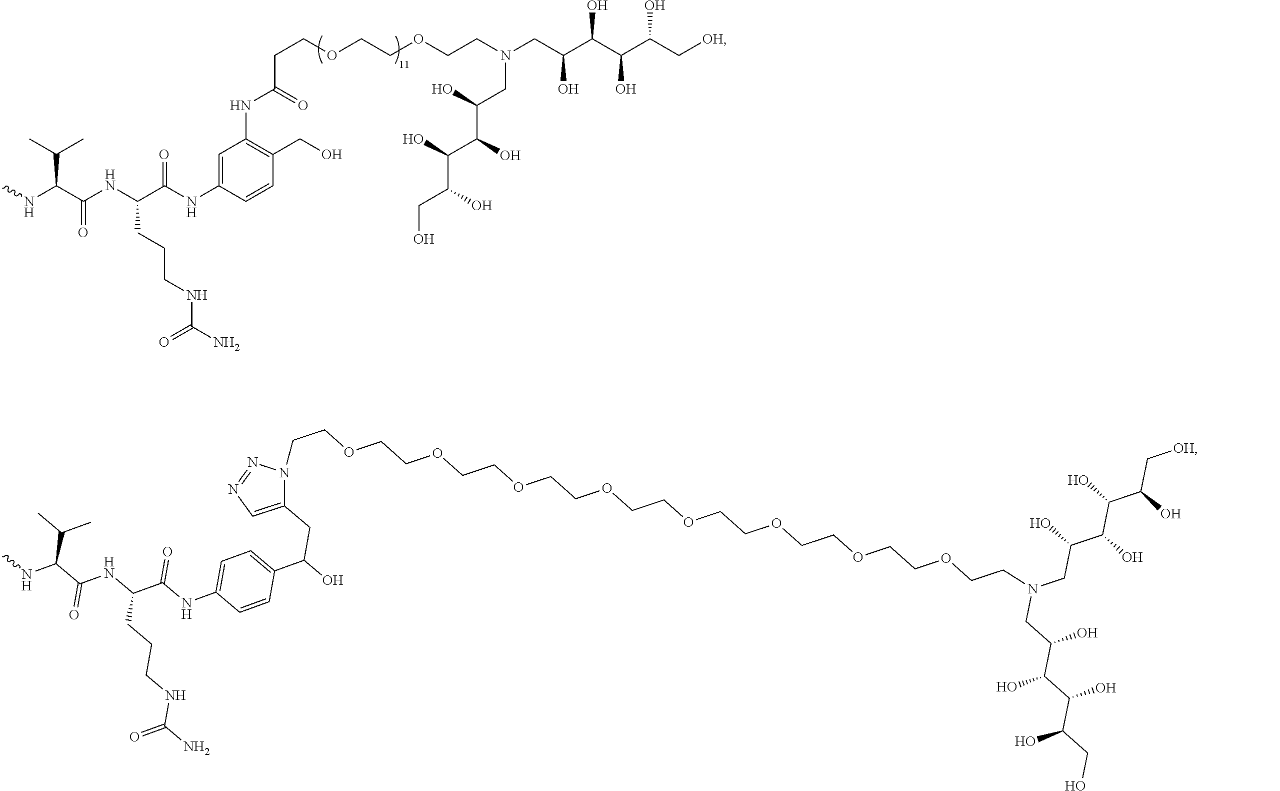

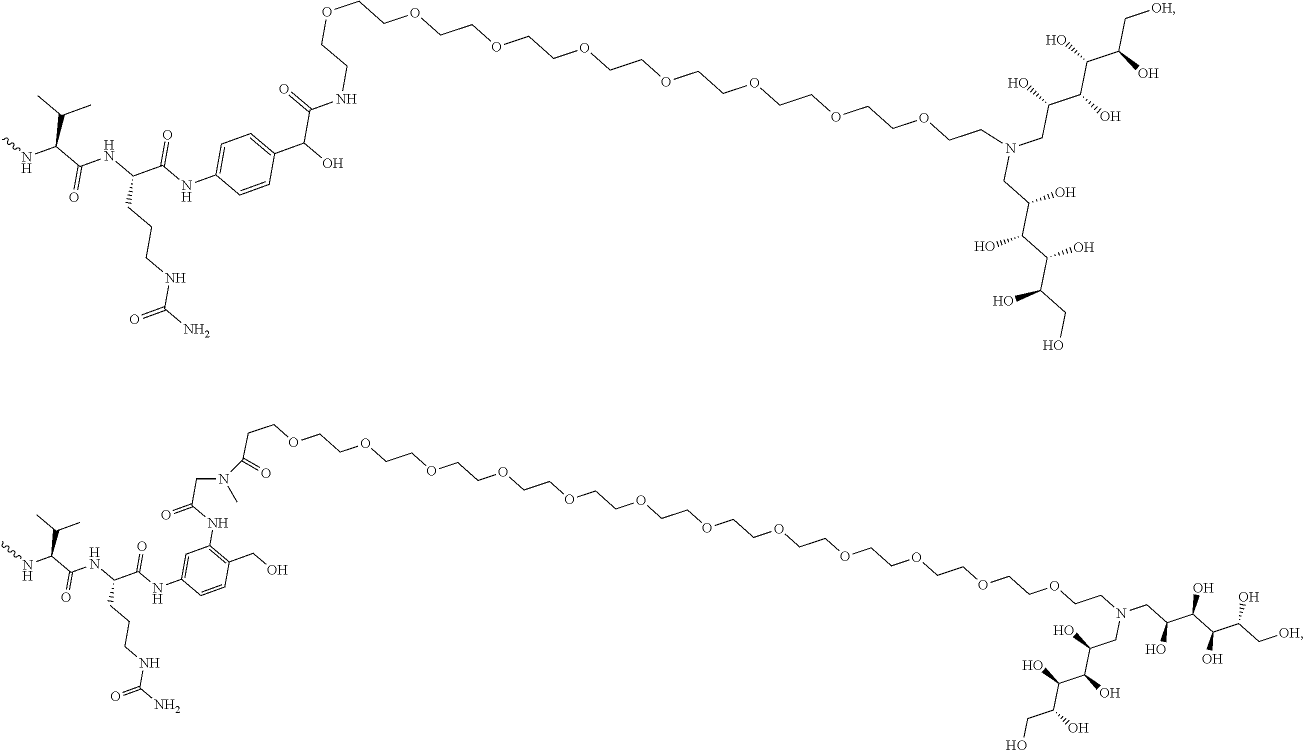

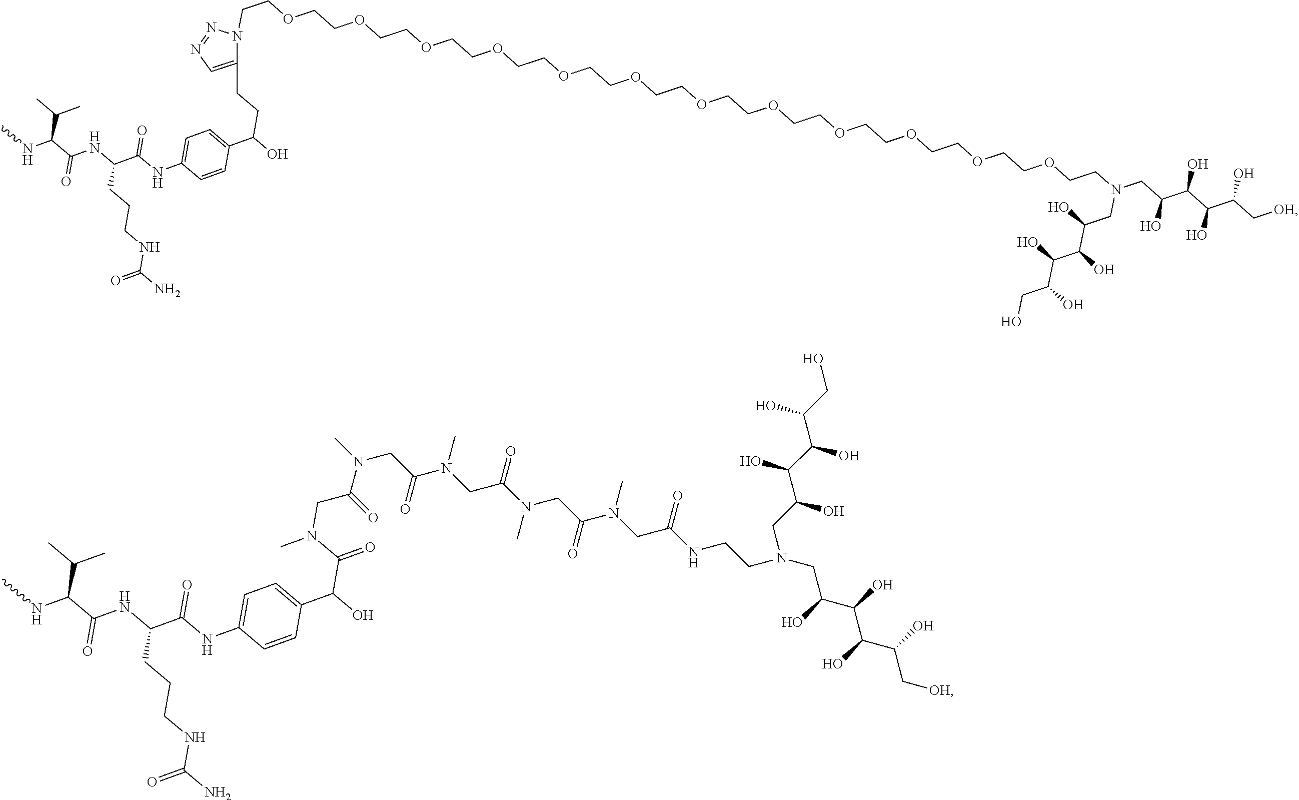

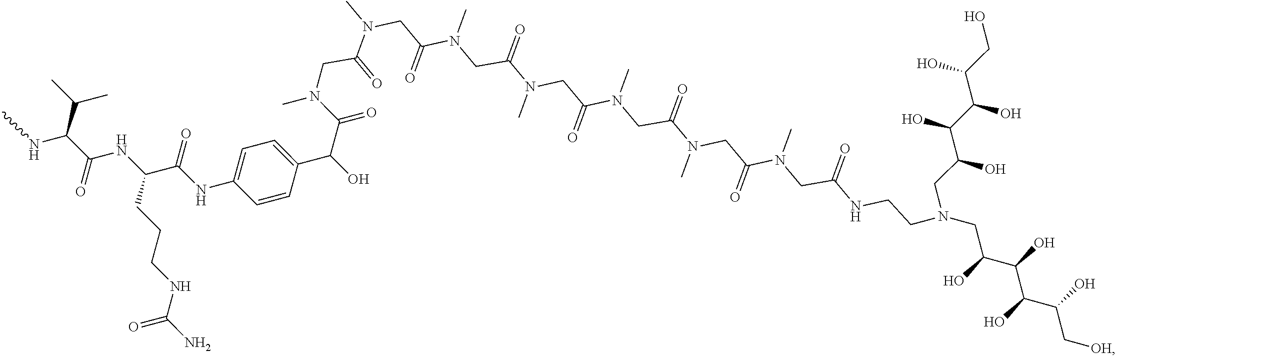

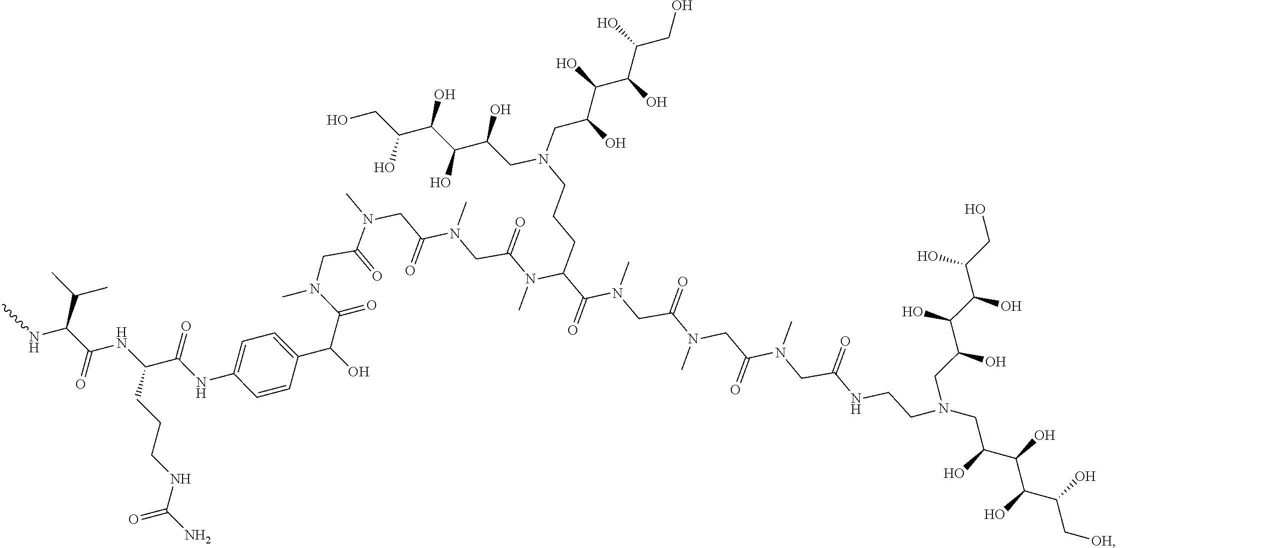

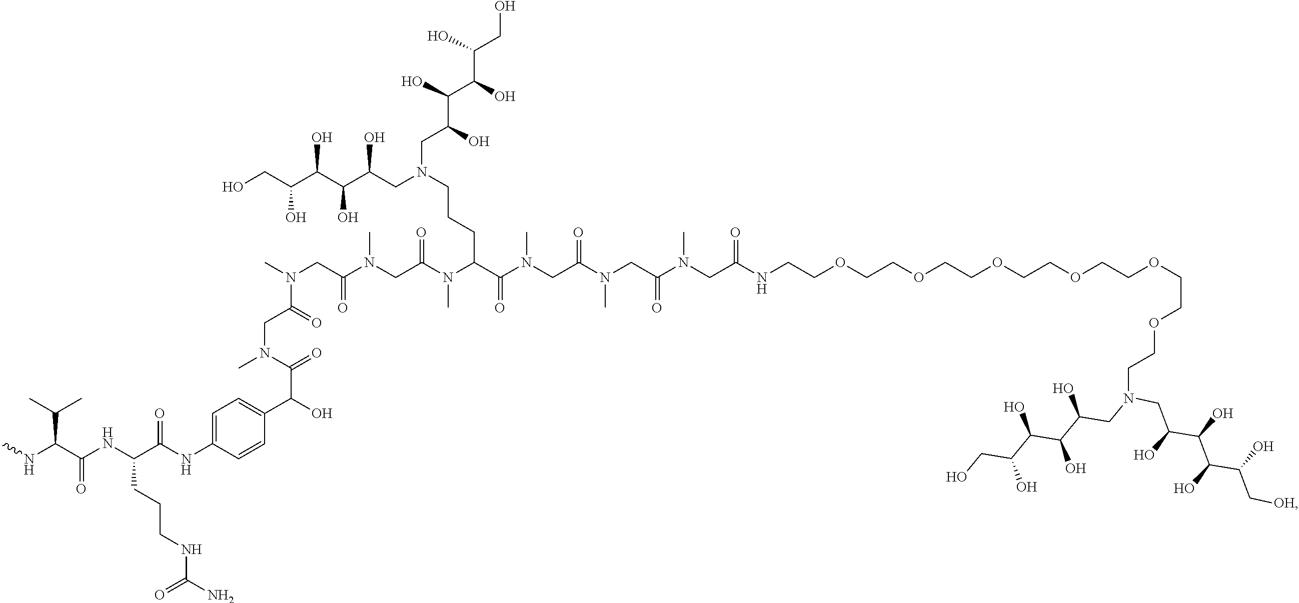

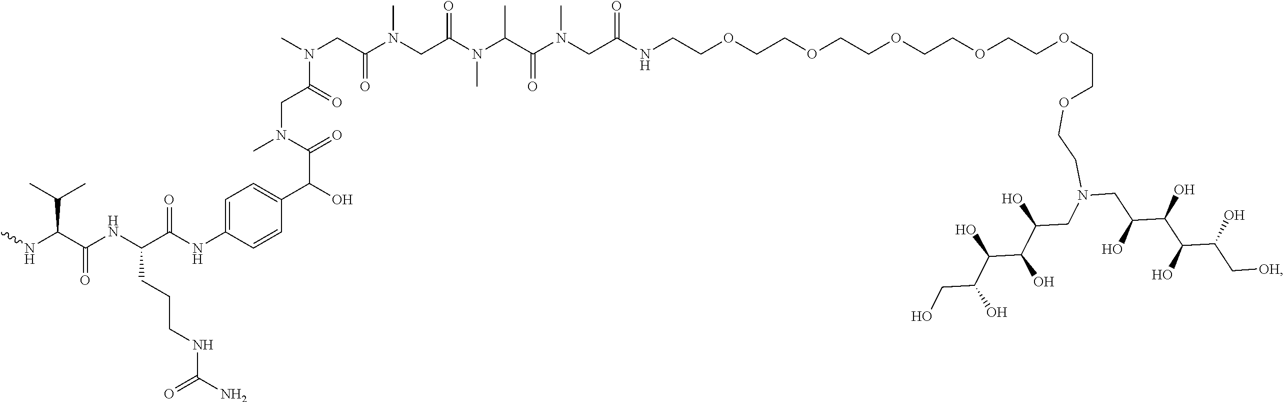

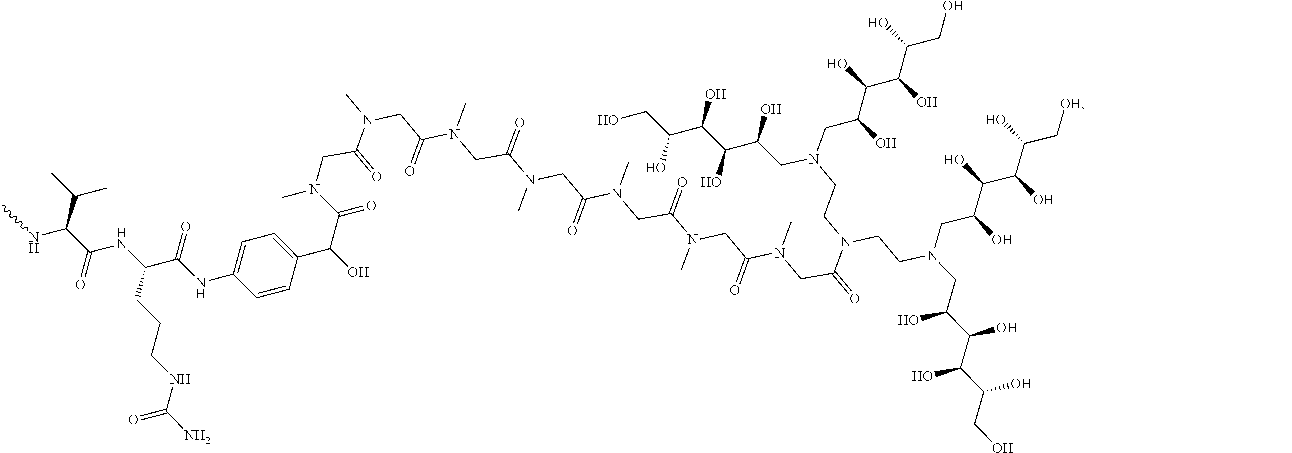

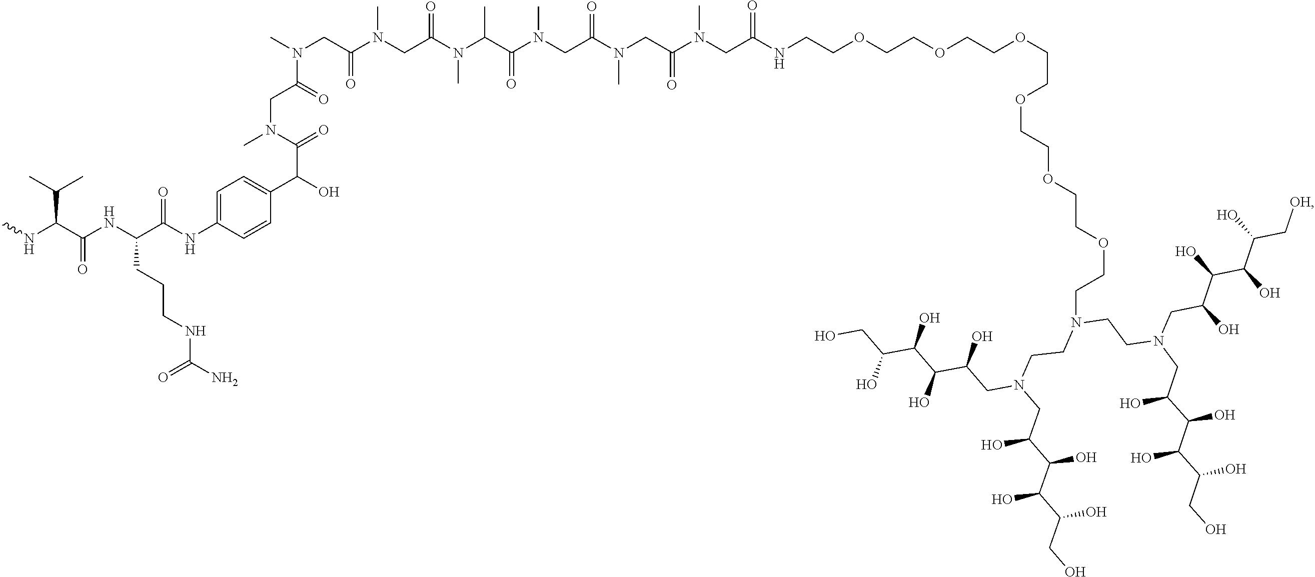

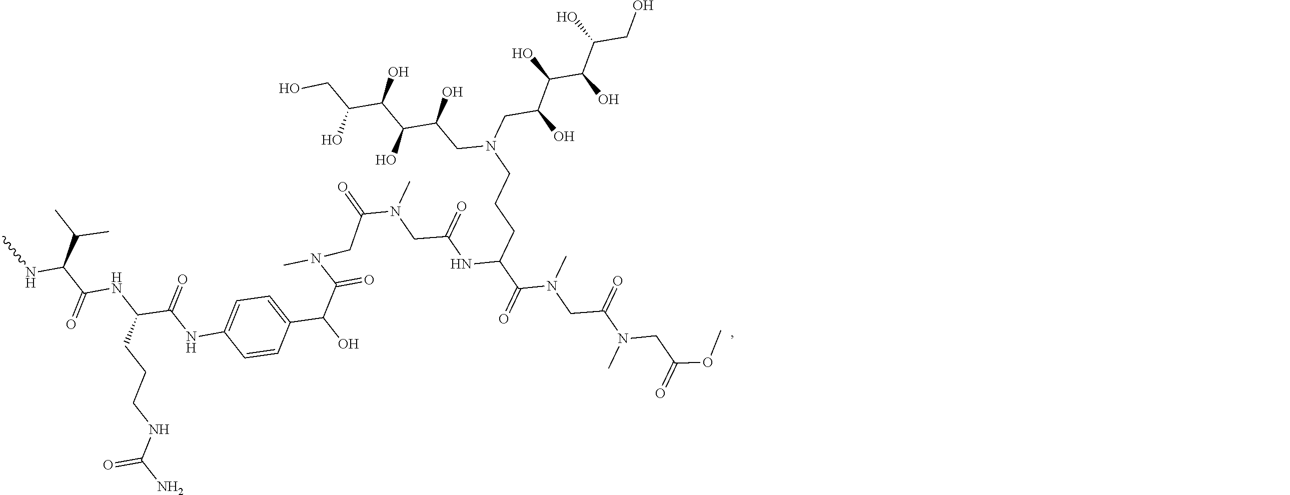

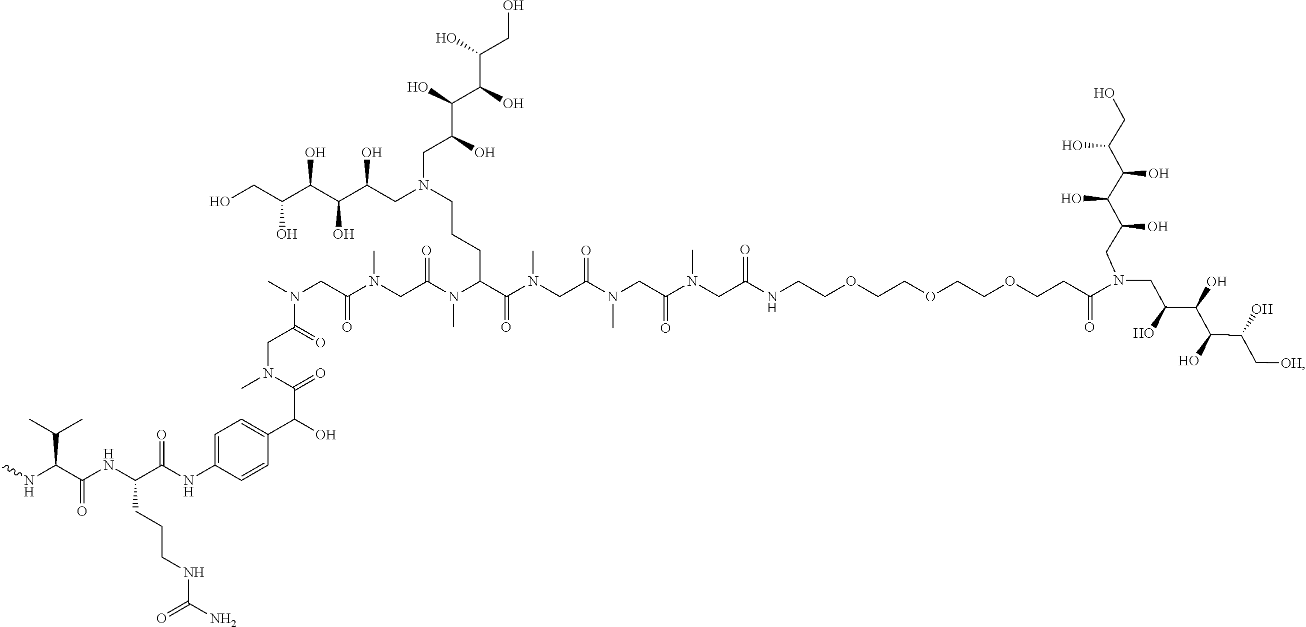

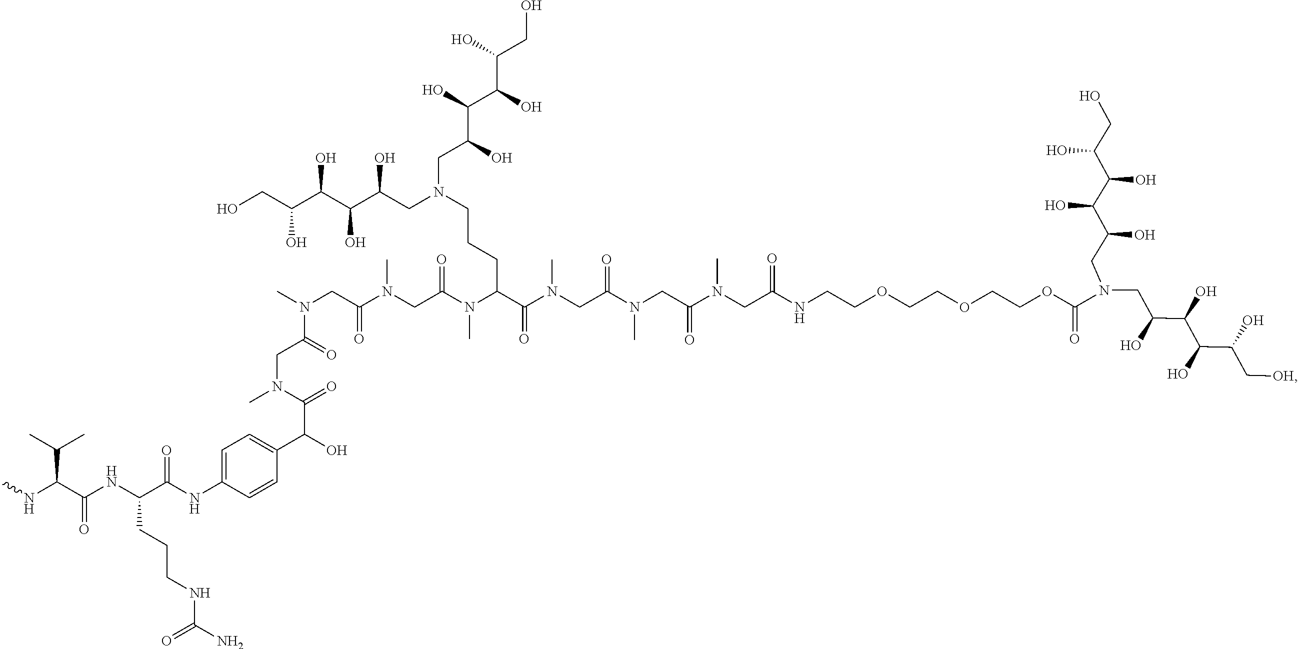

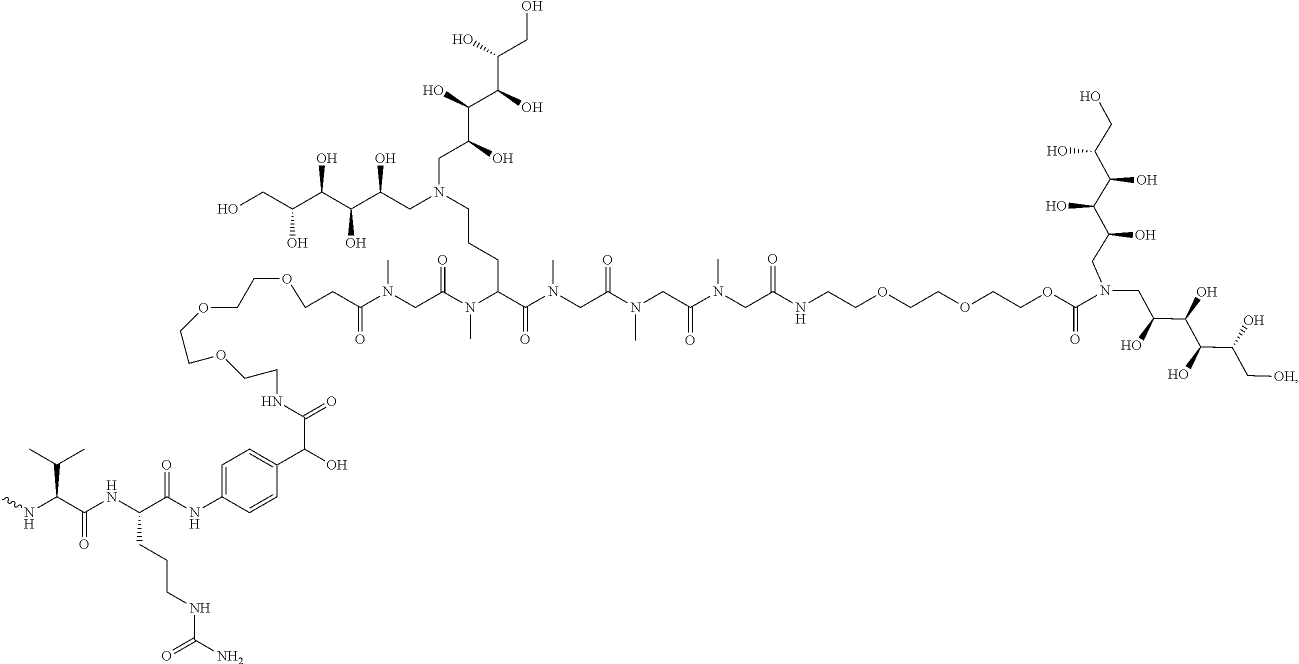

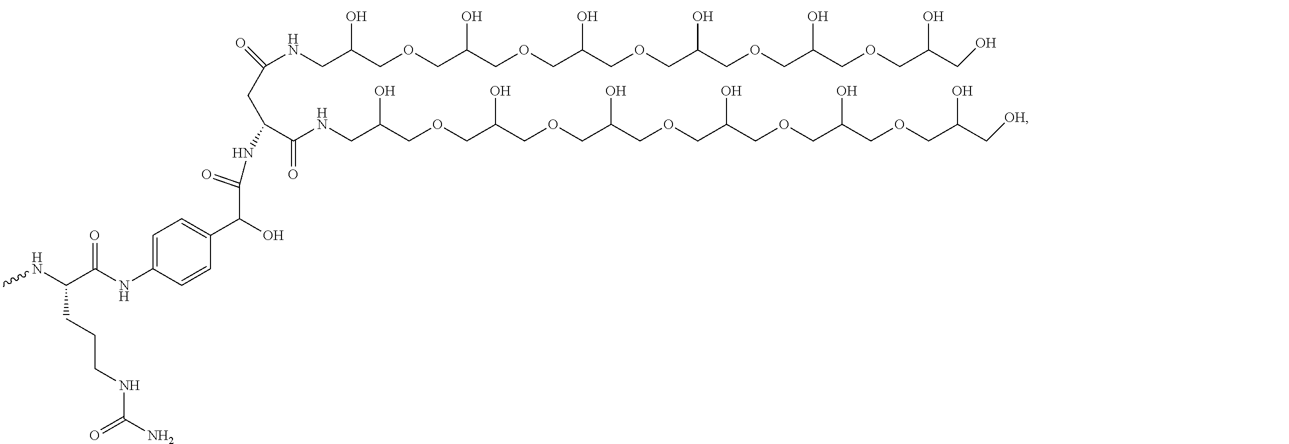

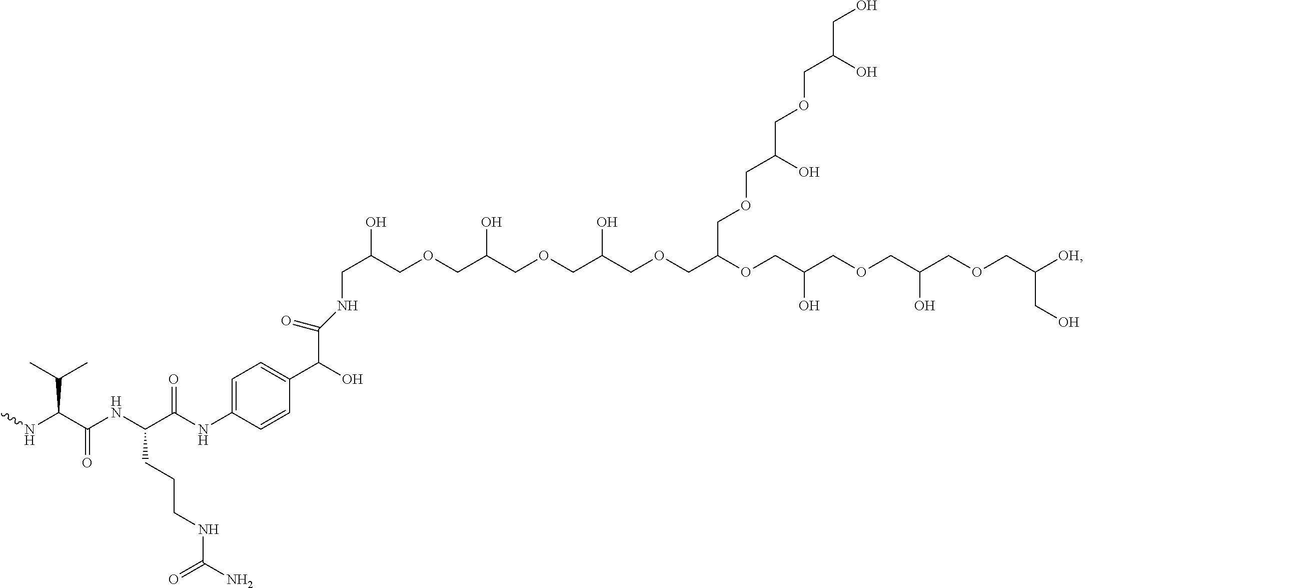

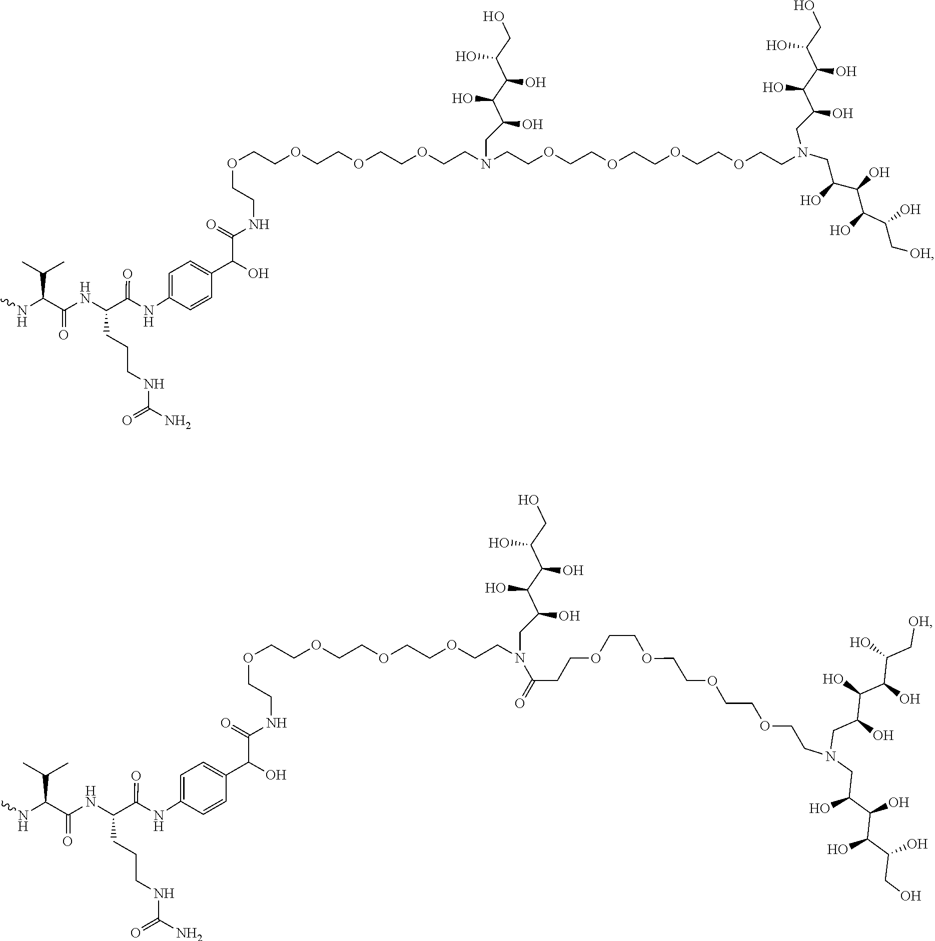

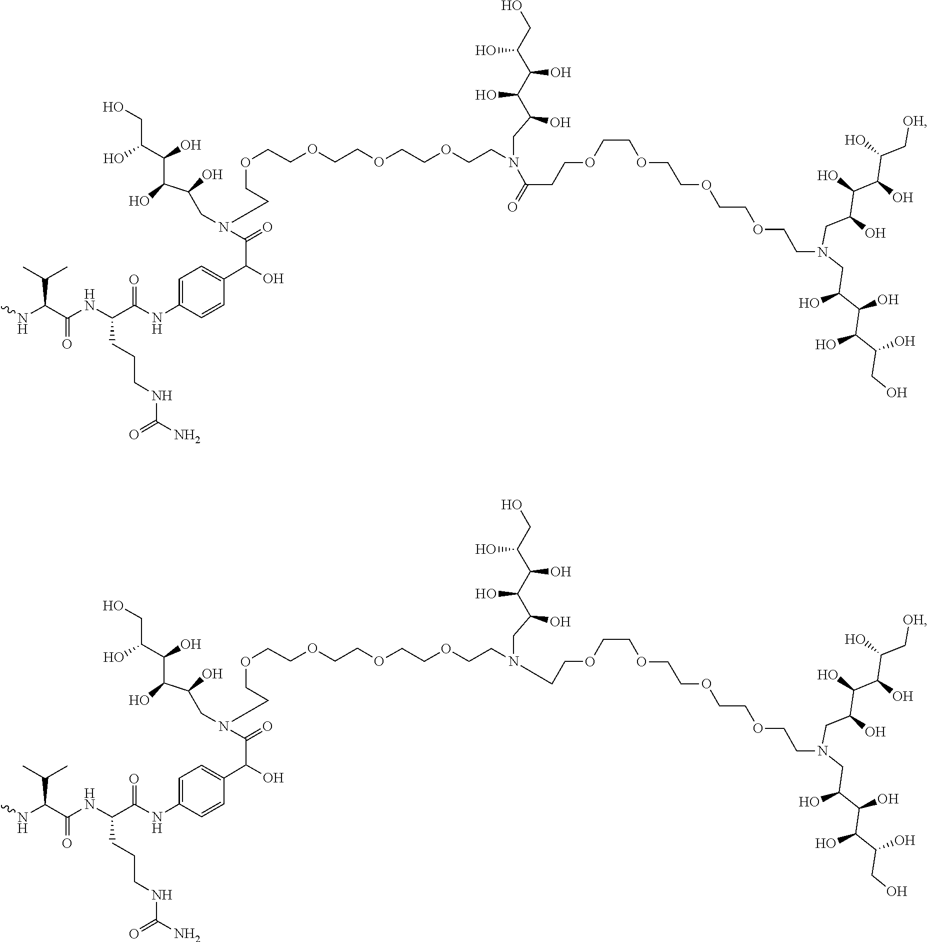

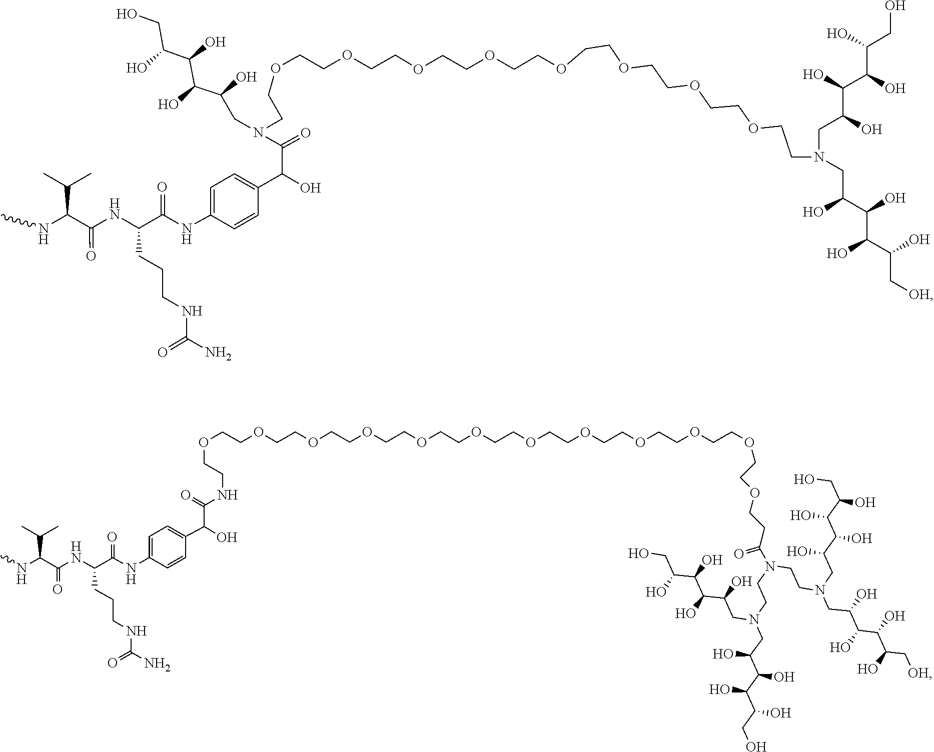

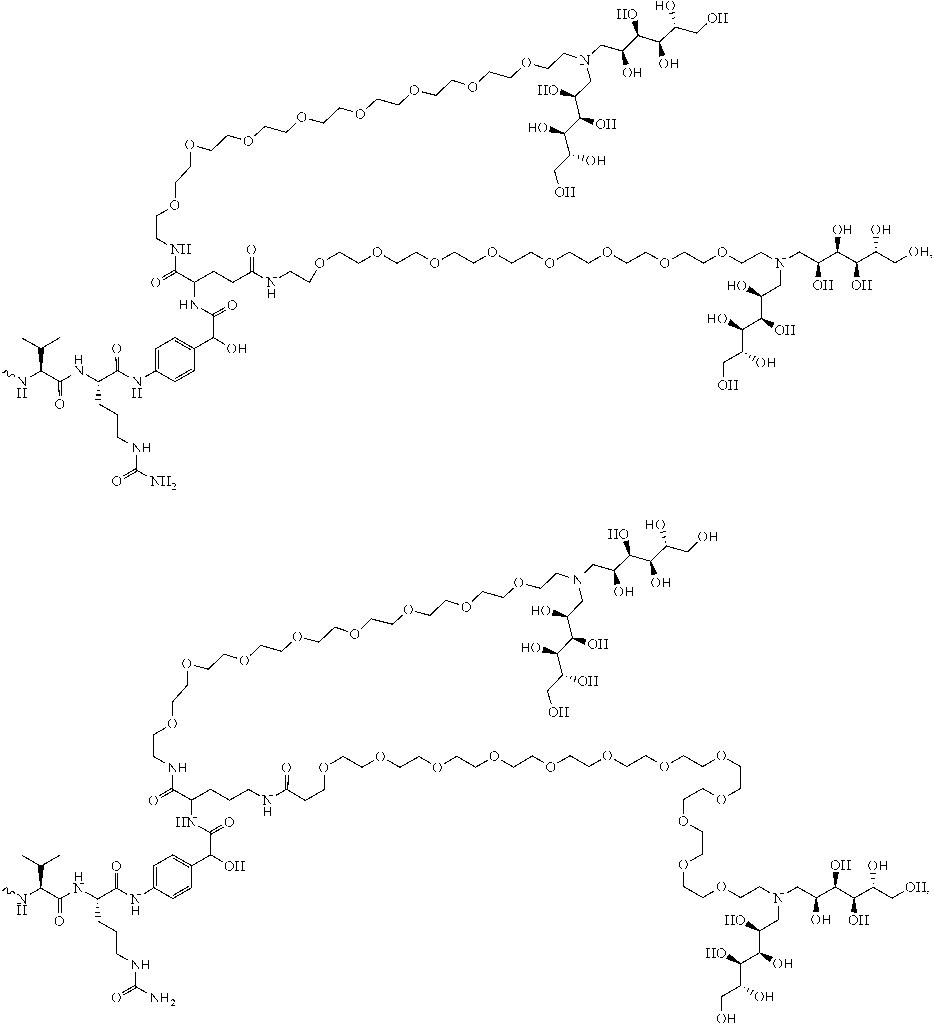

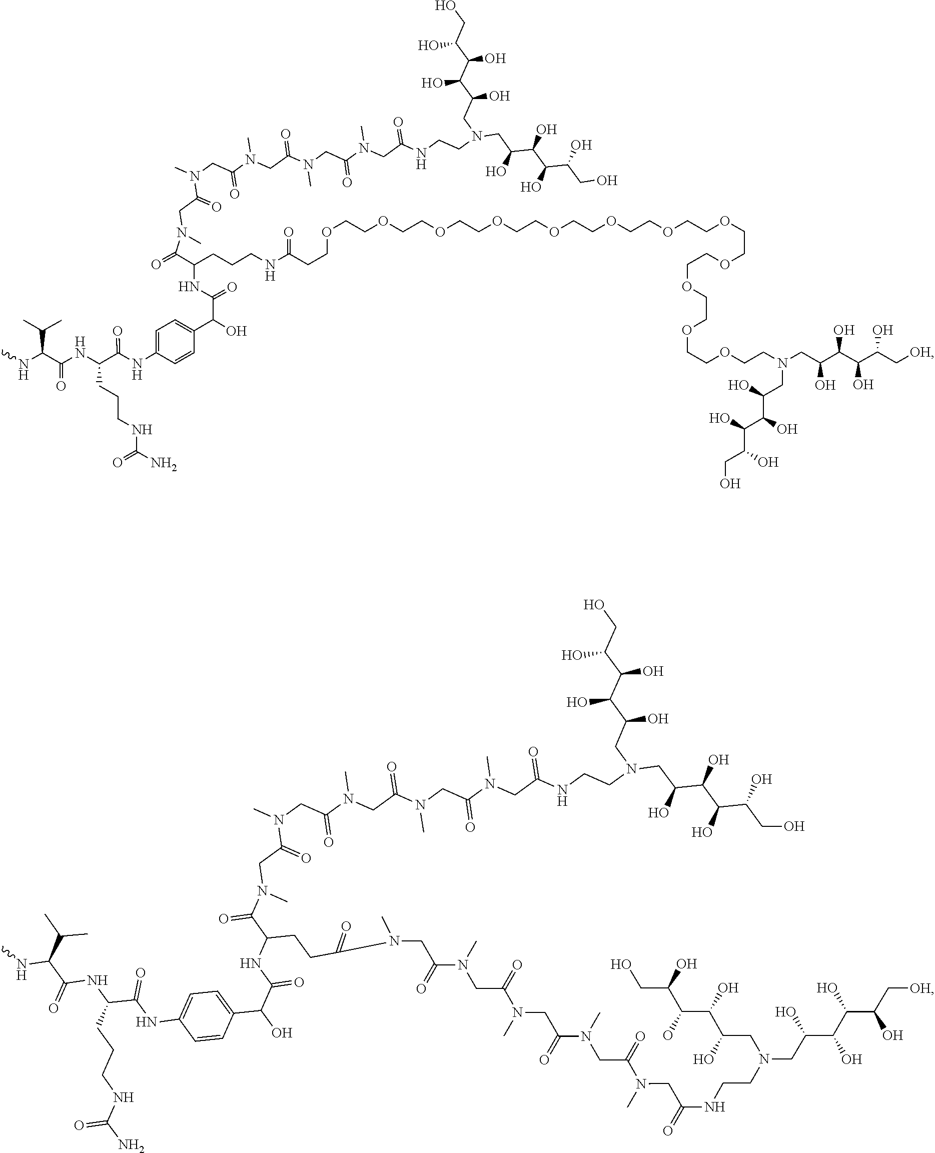

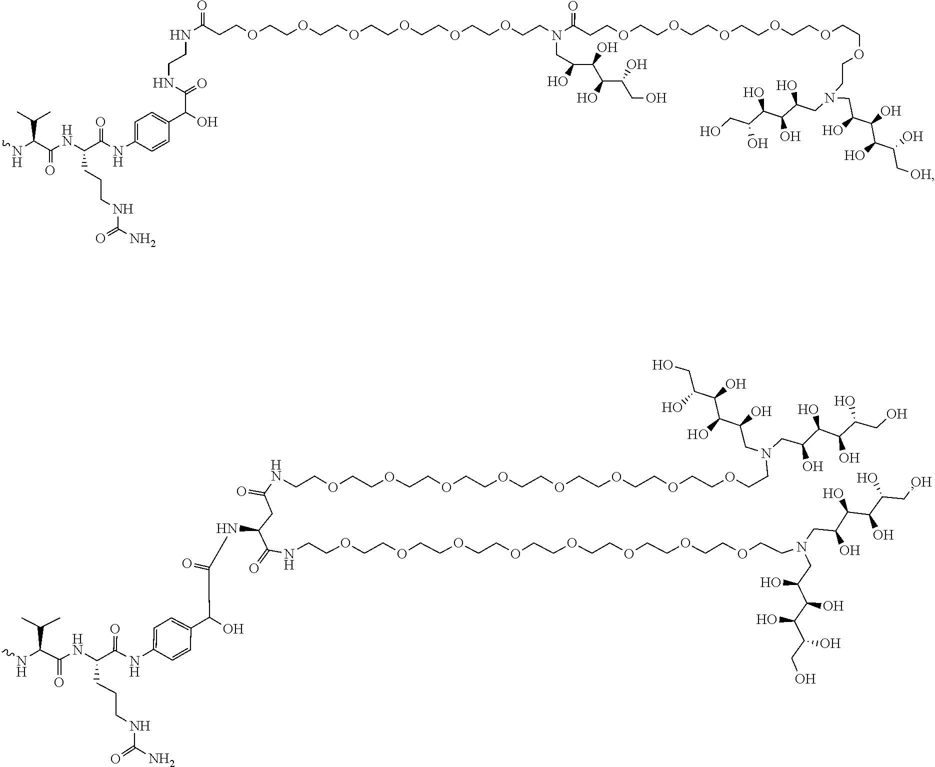

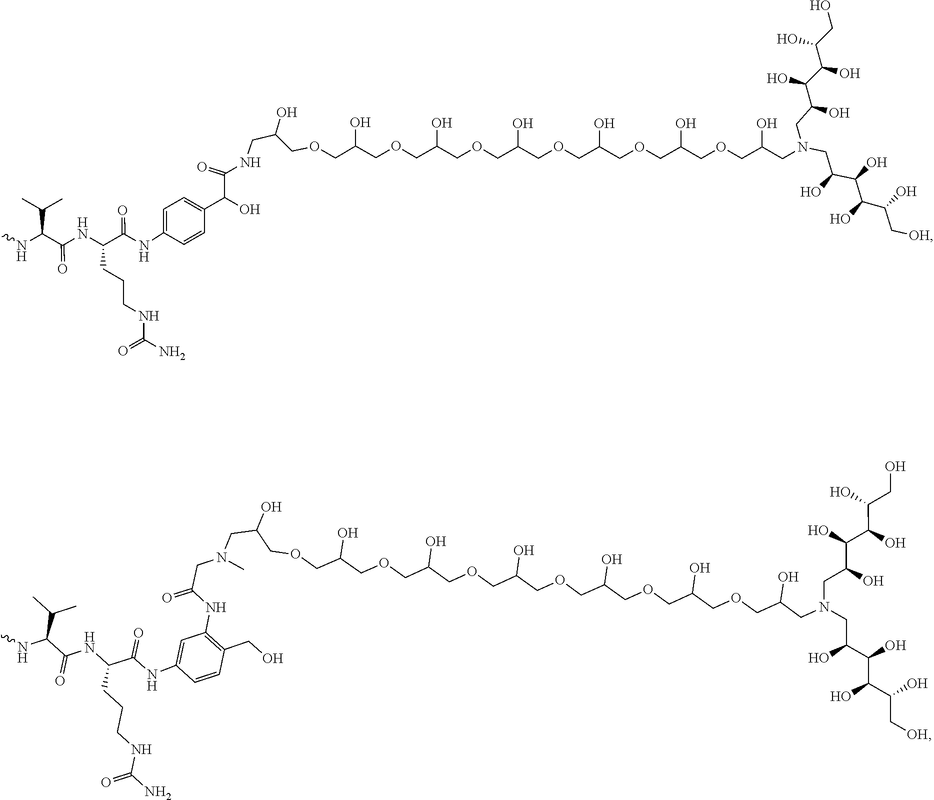

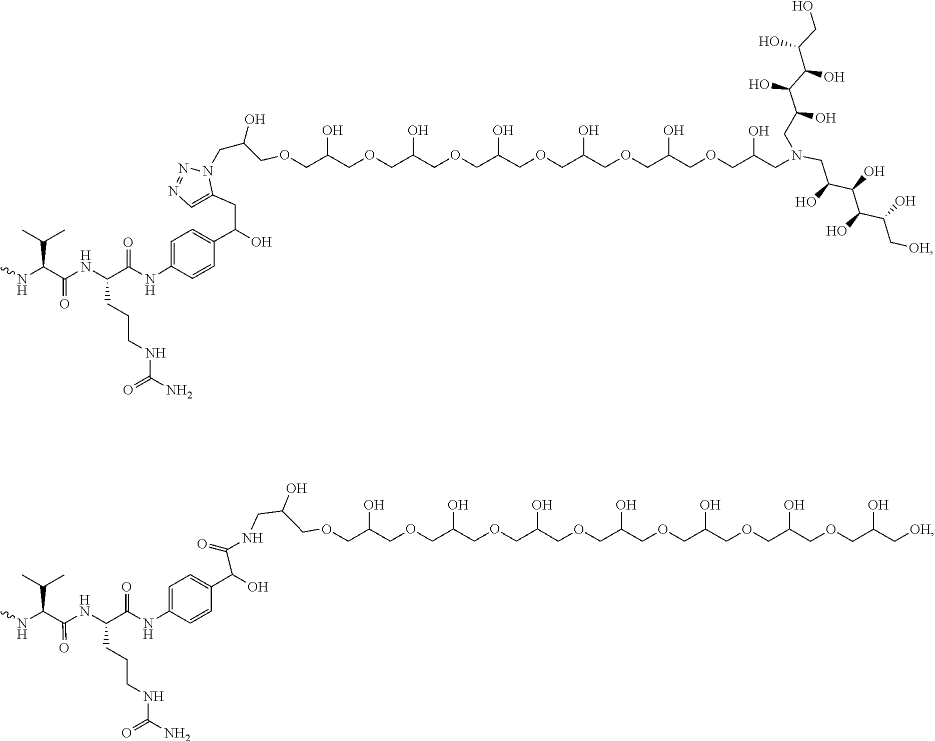

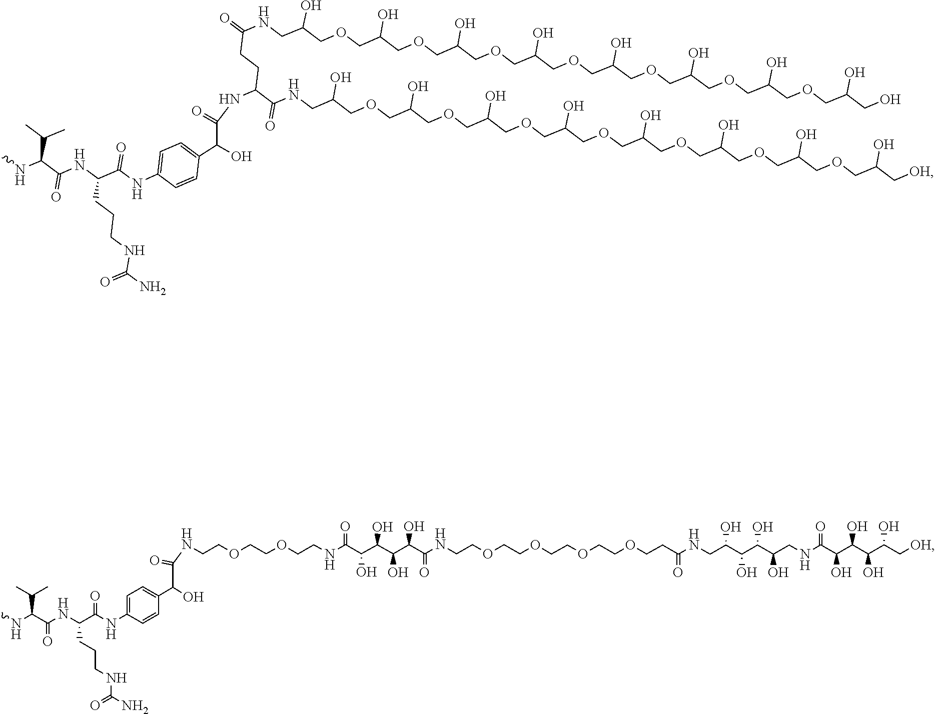

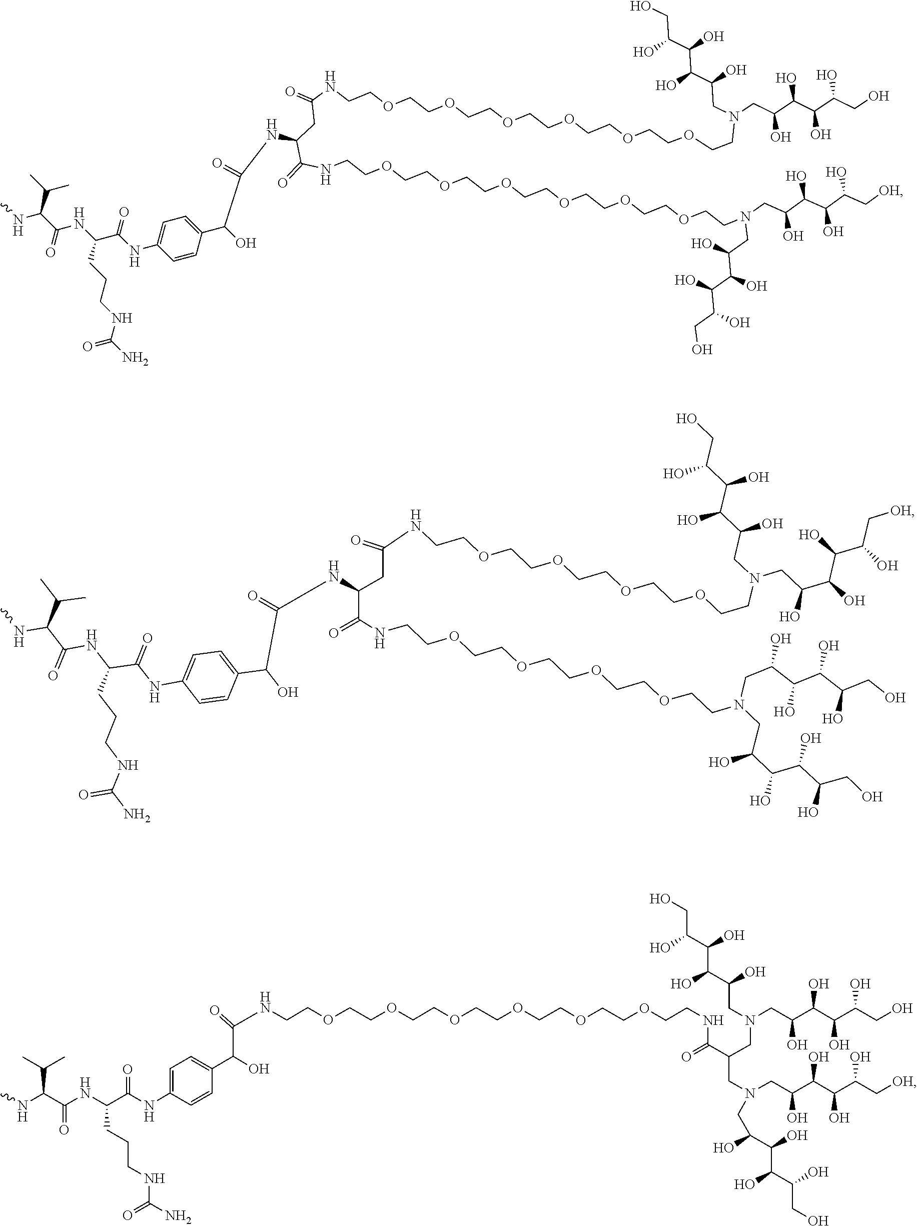

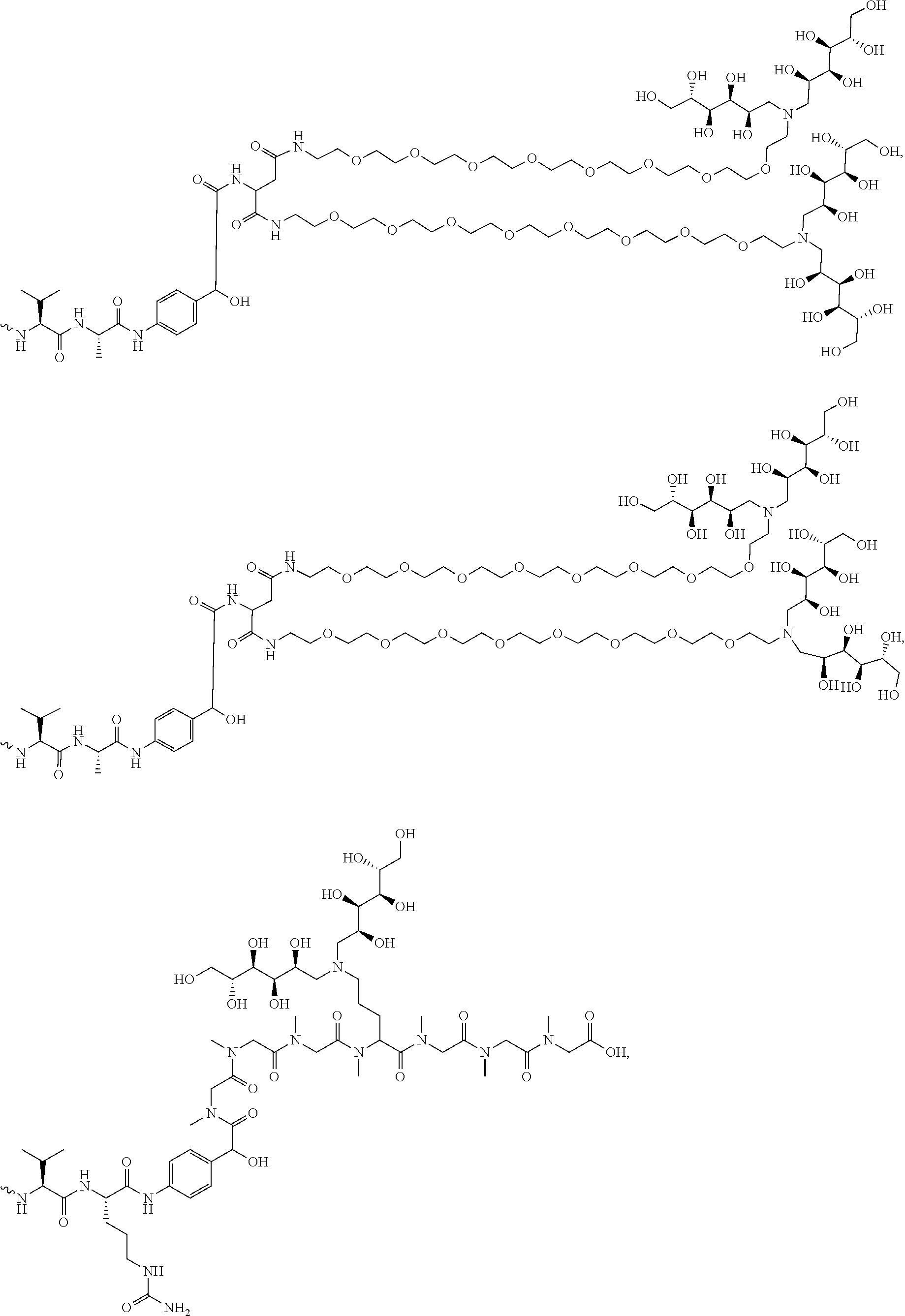

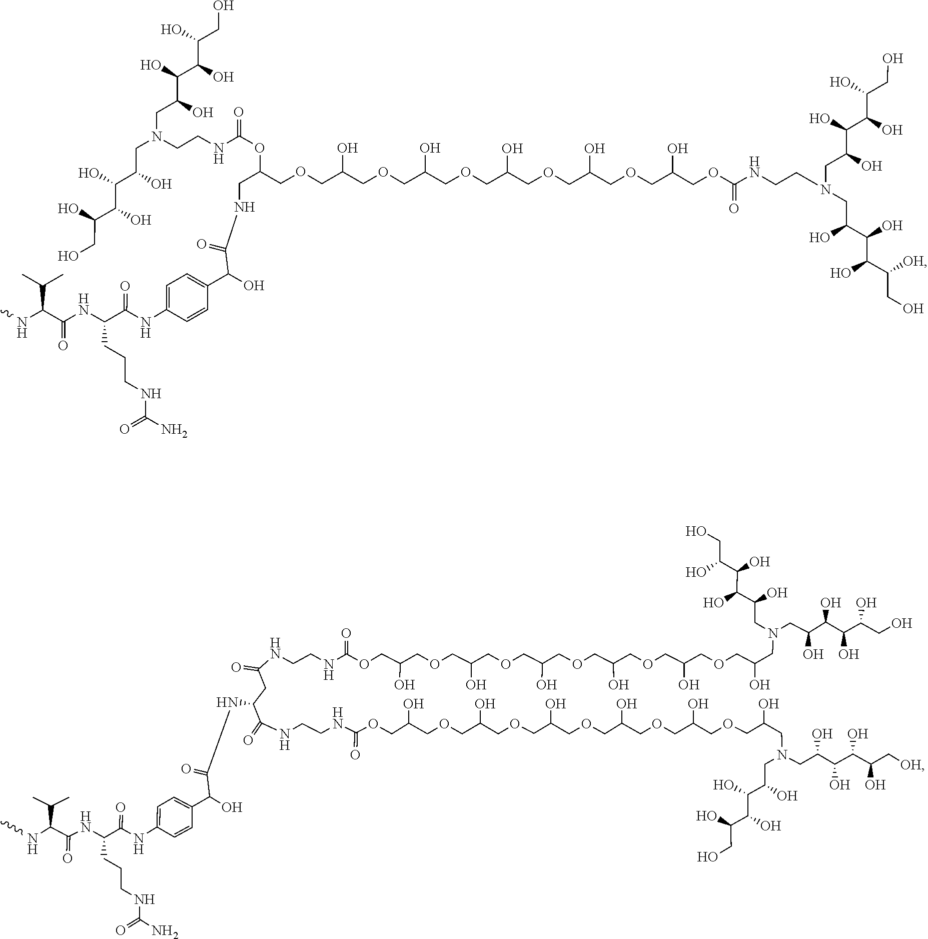

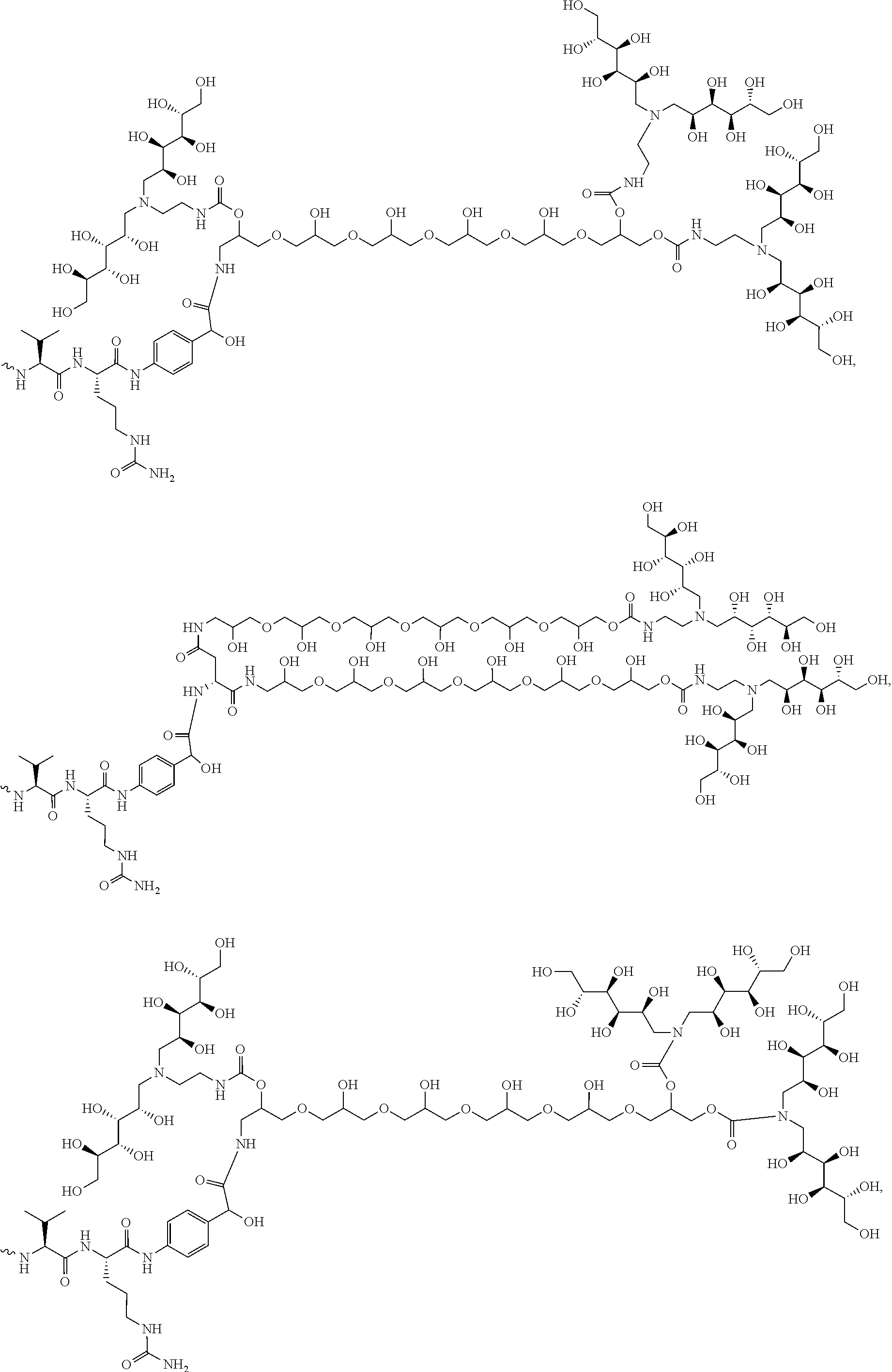

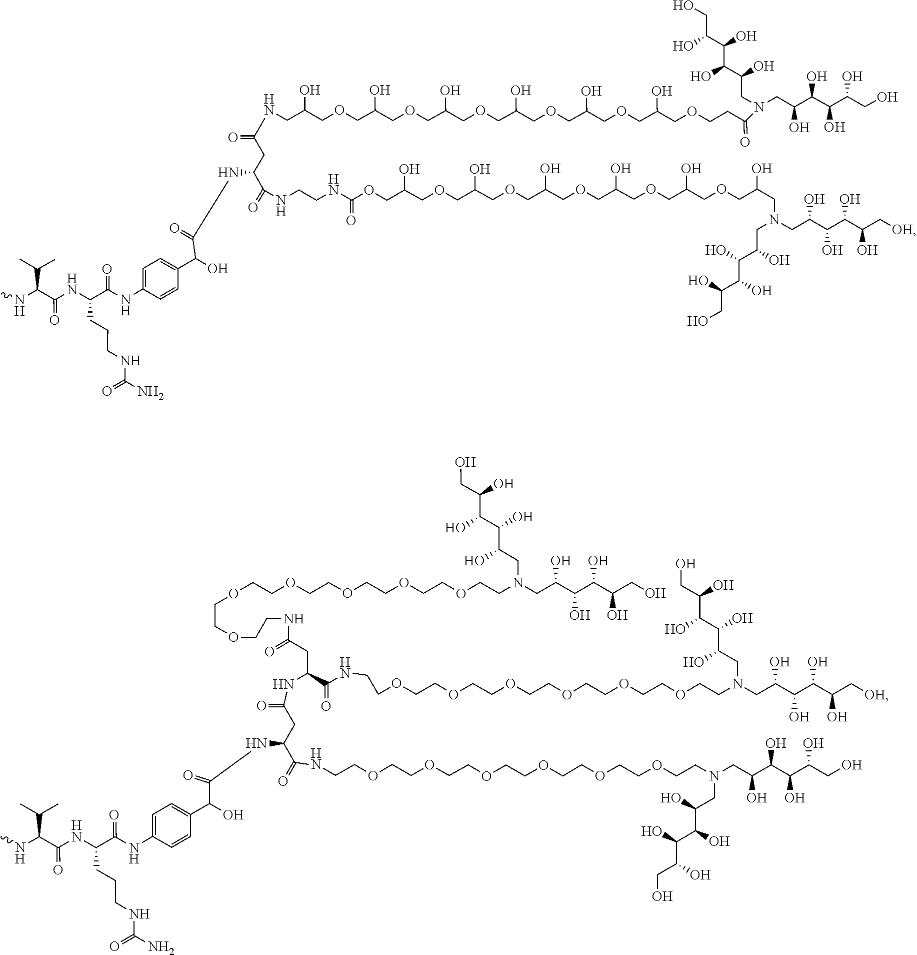

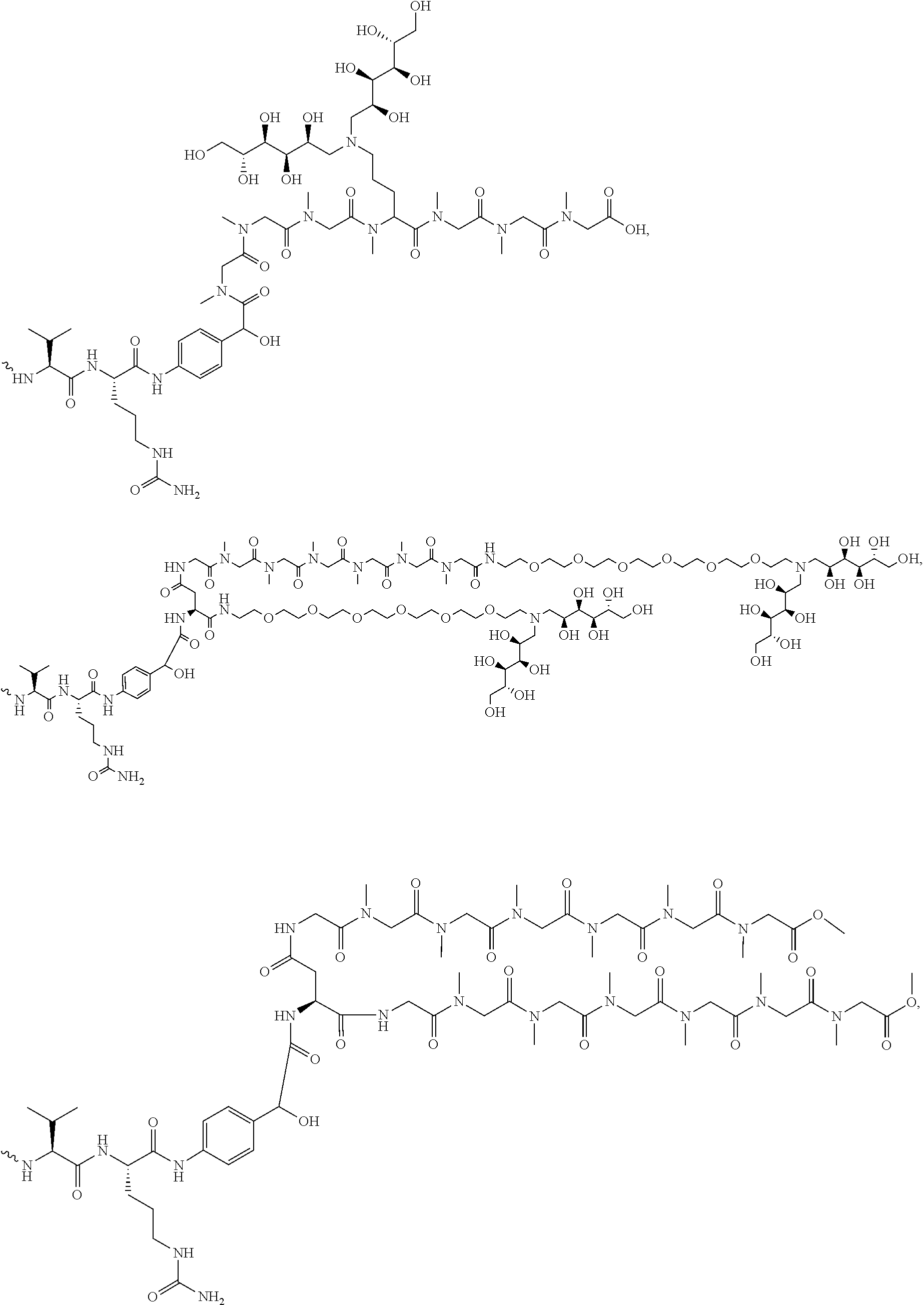

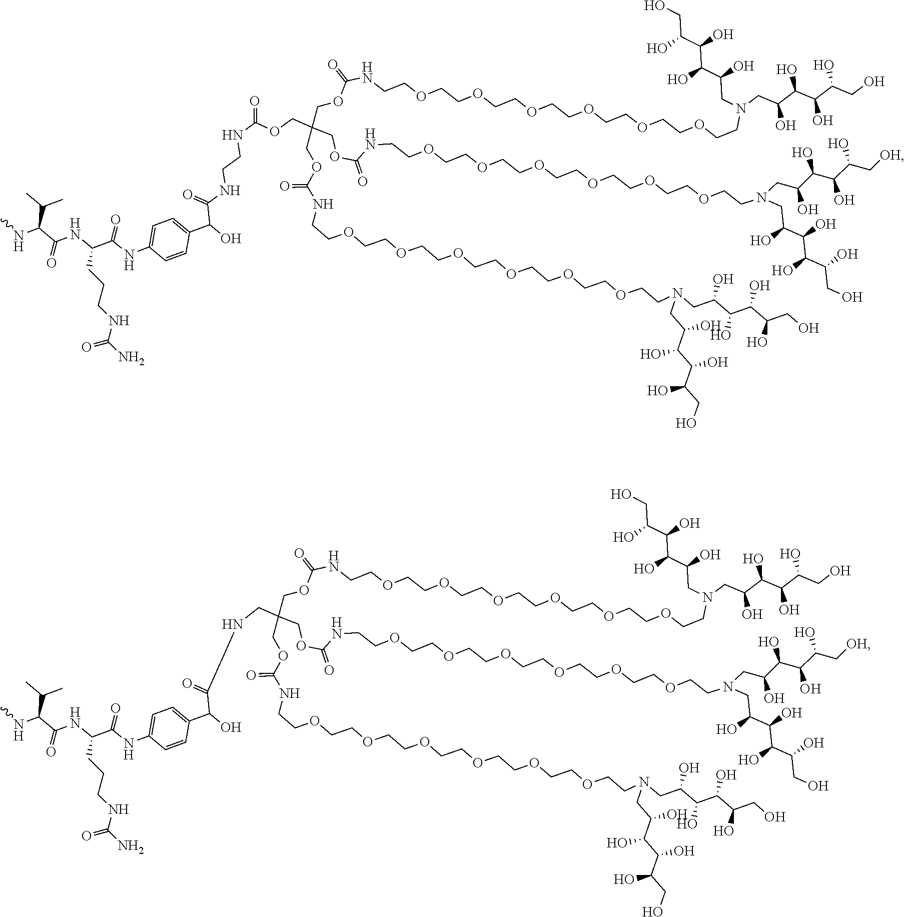

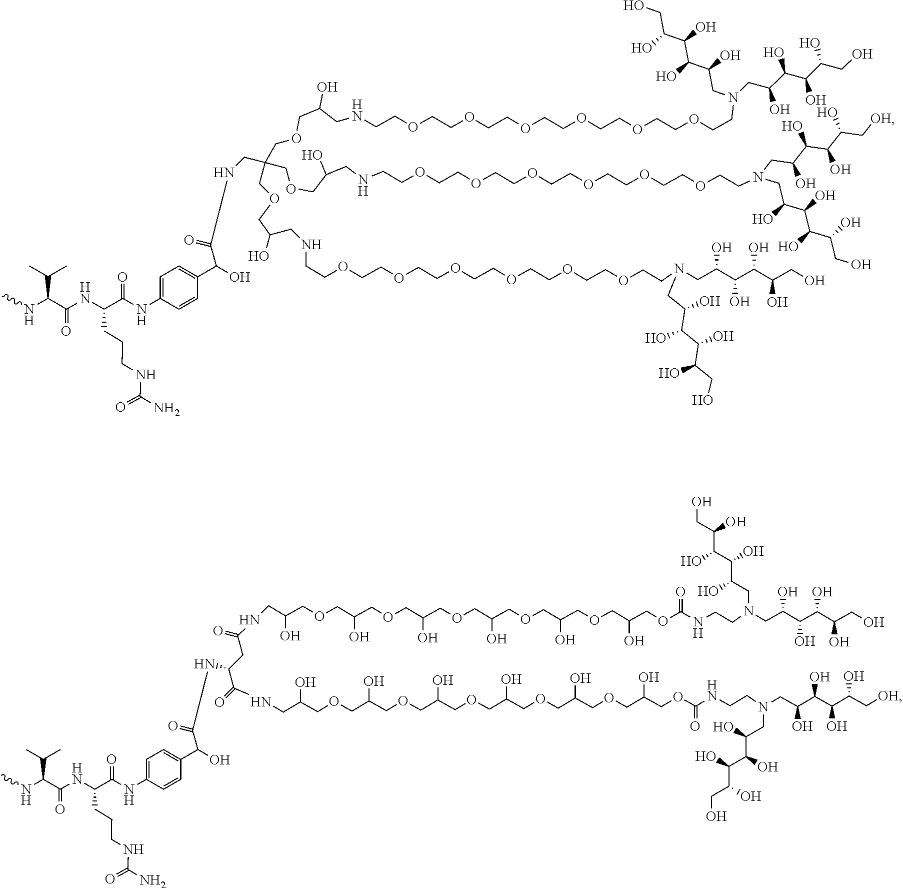

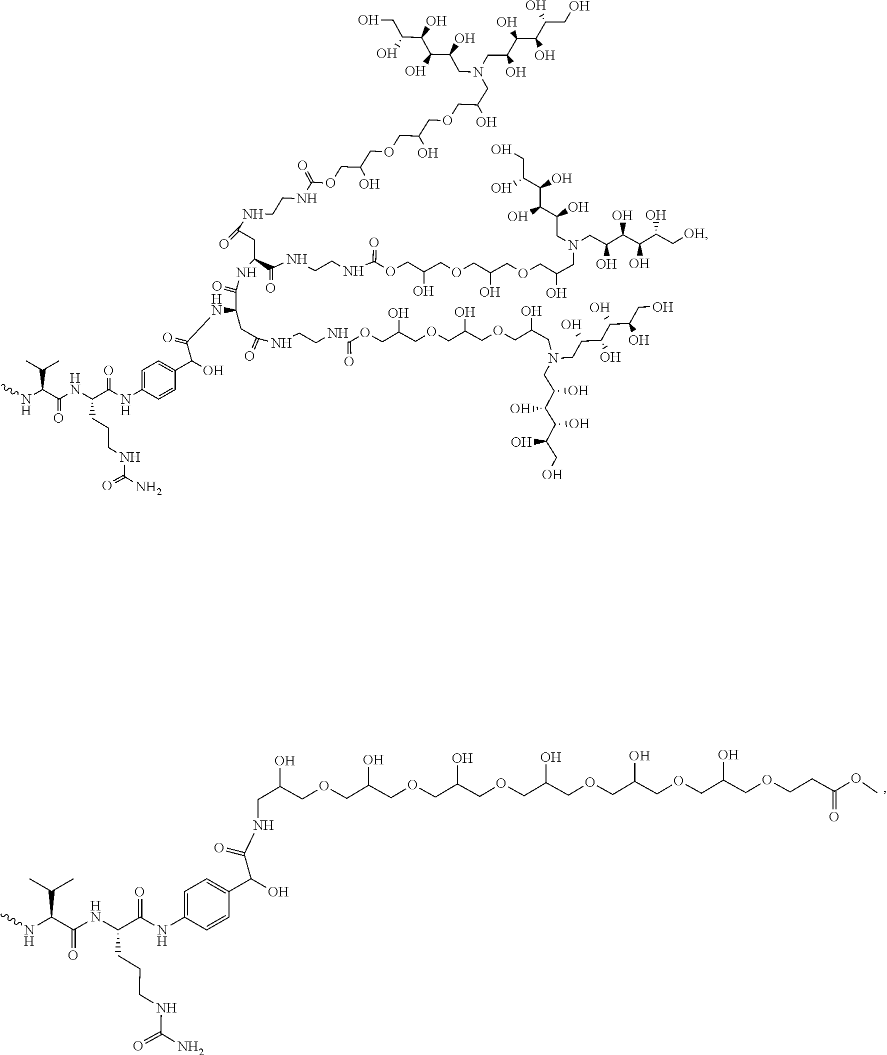

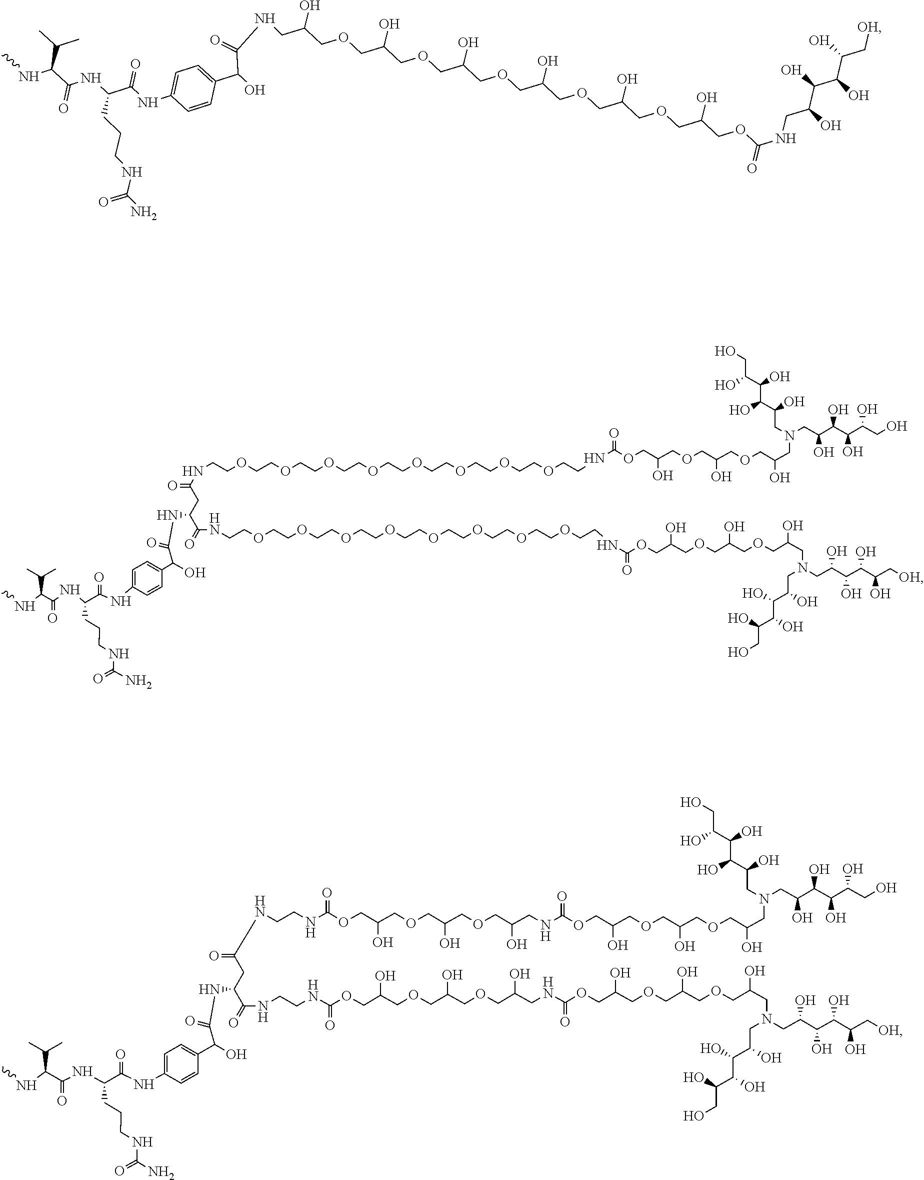

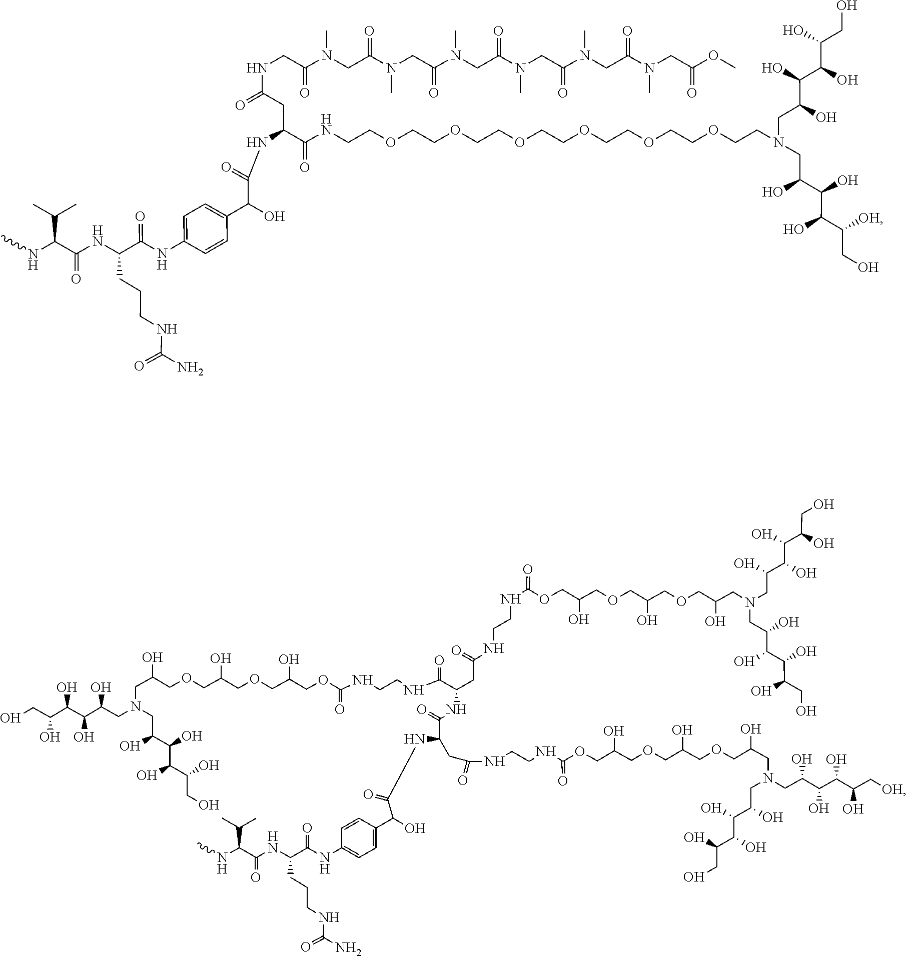

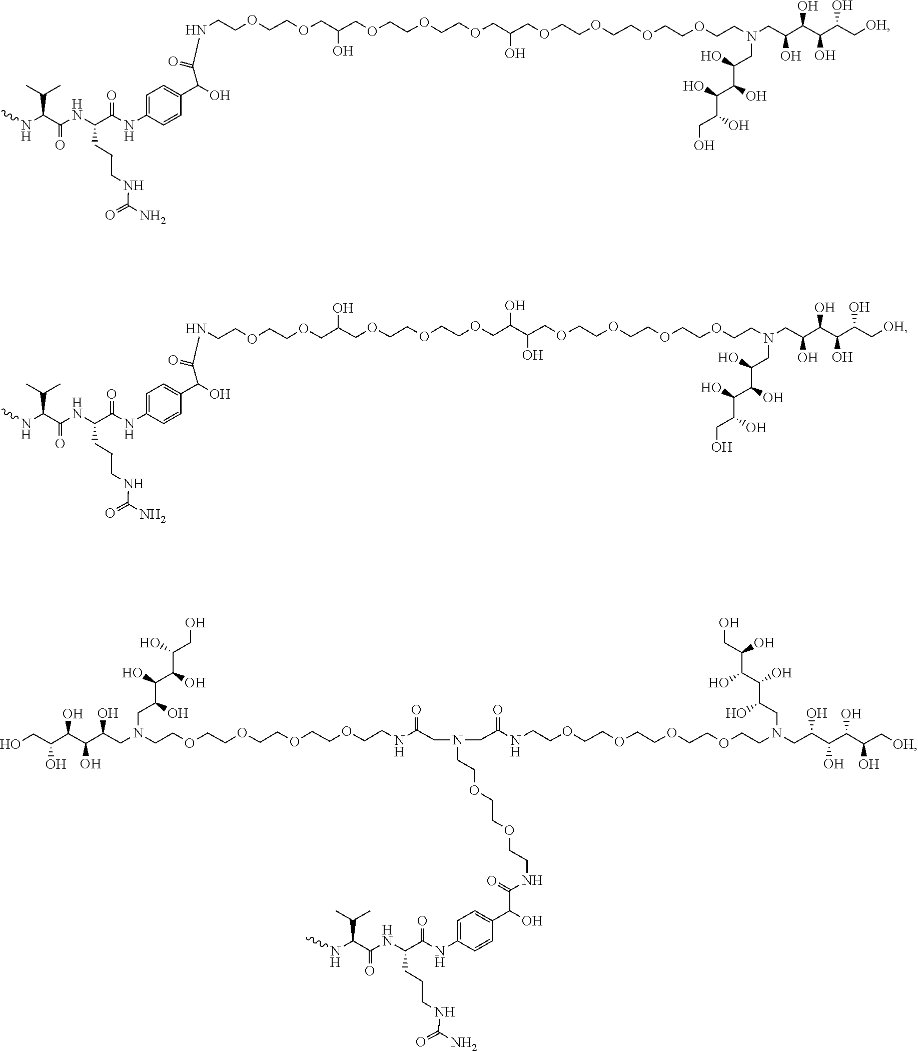

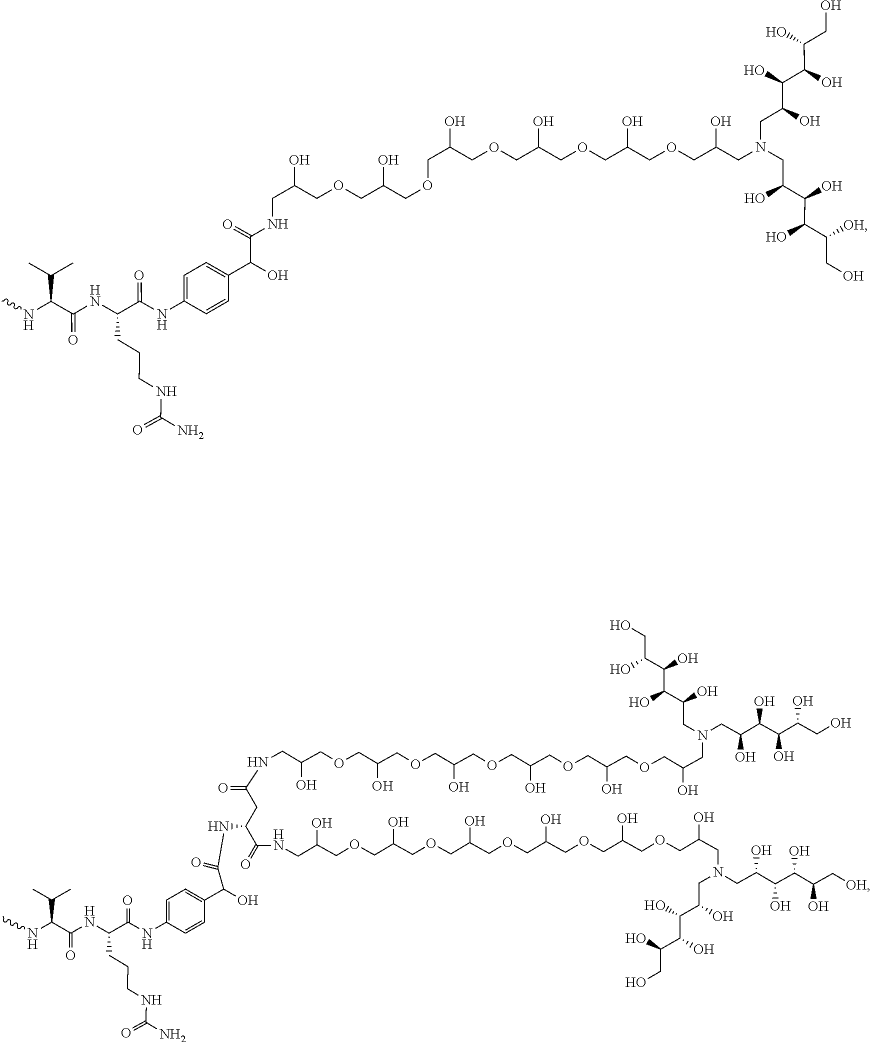

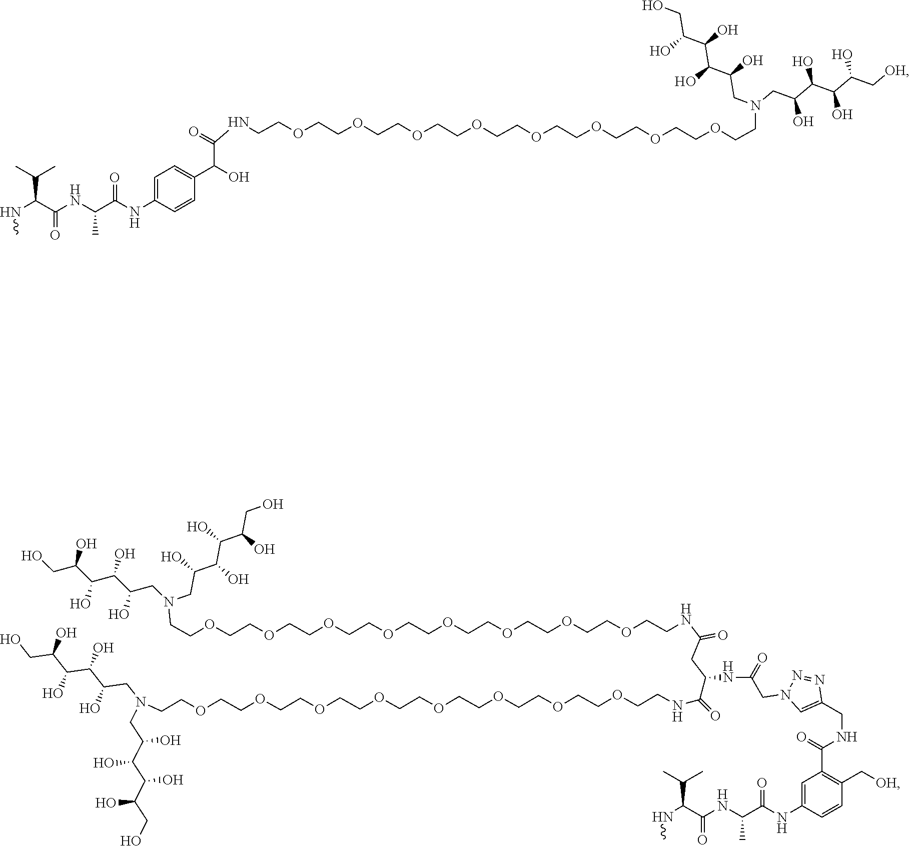

wherein the wavy line on the amino group indicates an attachment site to the stretcher group;
or, prior to attachment to the stretcher group, indicates H, and the H on the benzylic OH is optionally replaced with a bond to at least one of the drug units or a linking group attached to the at least one of the drug units.

Embodiment 100. The linker compound of any one of Embodiments 1 to 14, comprising one of the following structures:

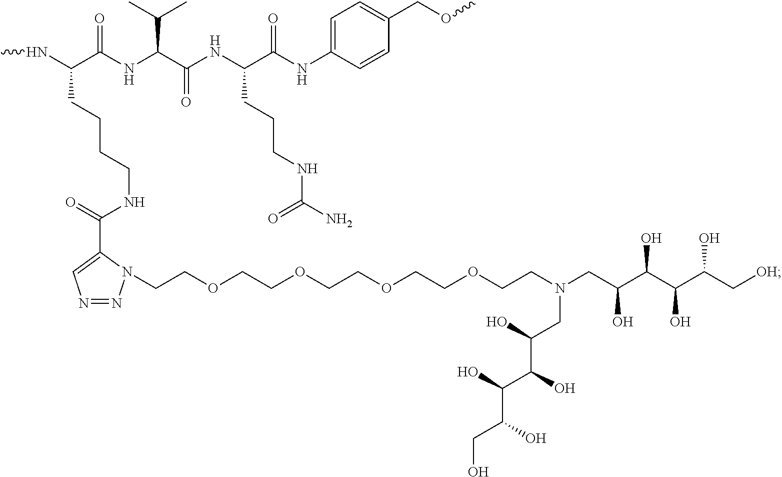

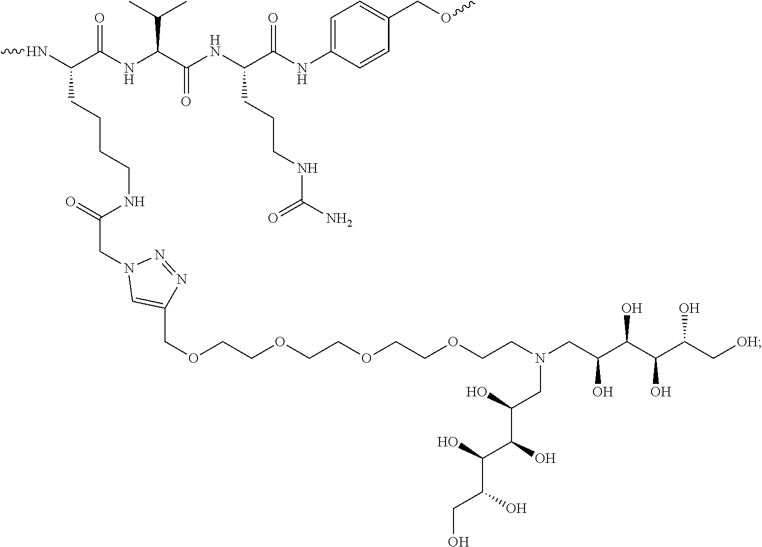

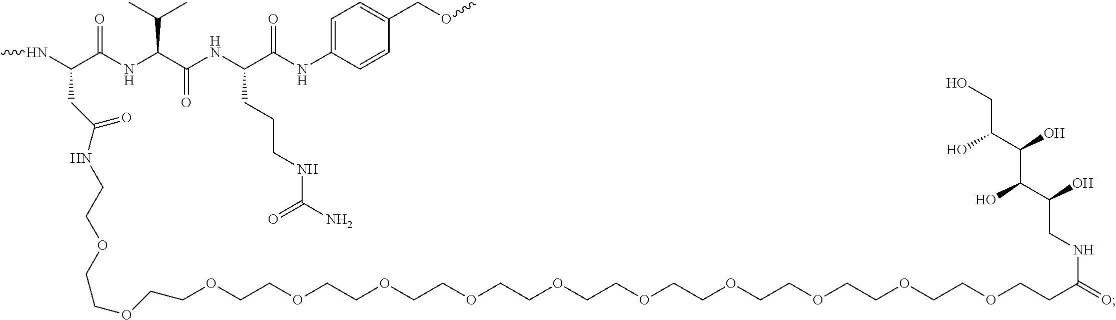

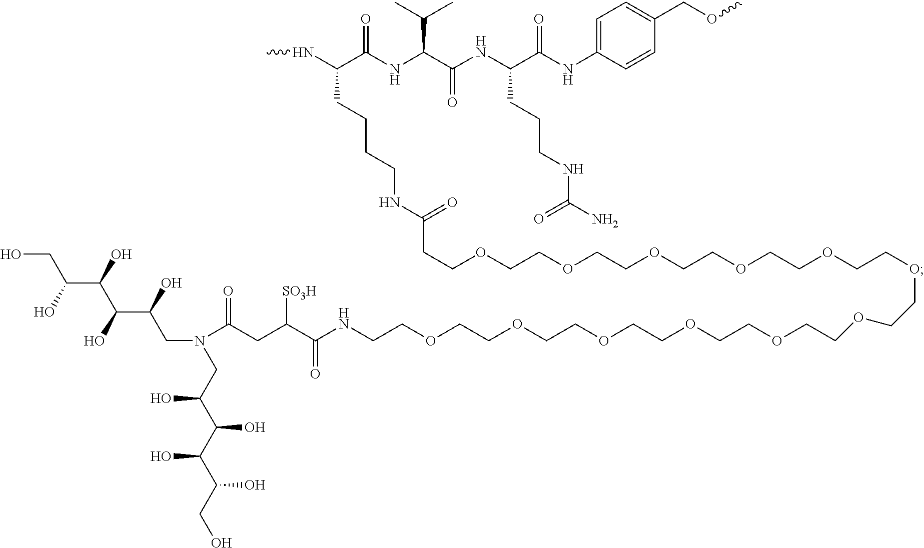

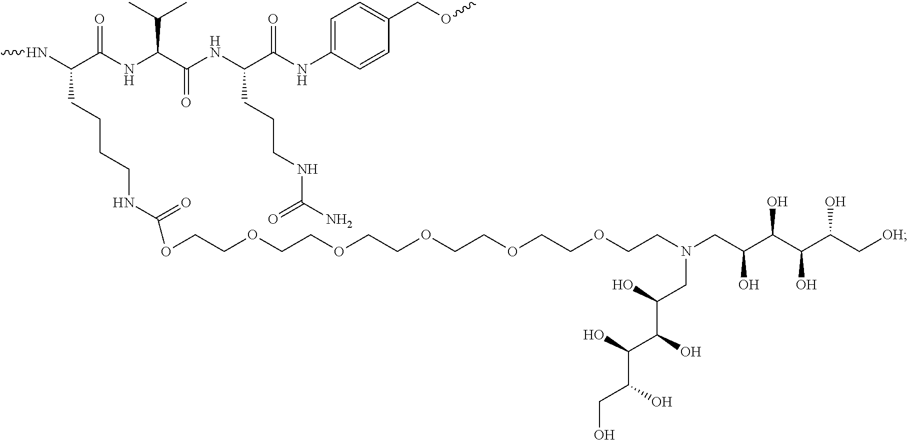

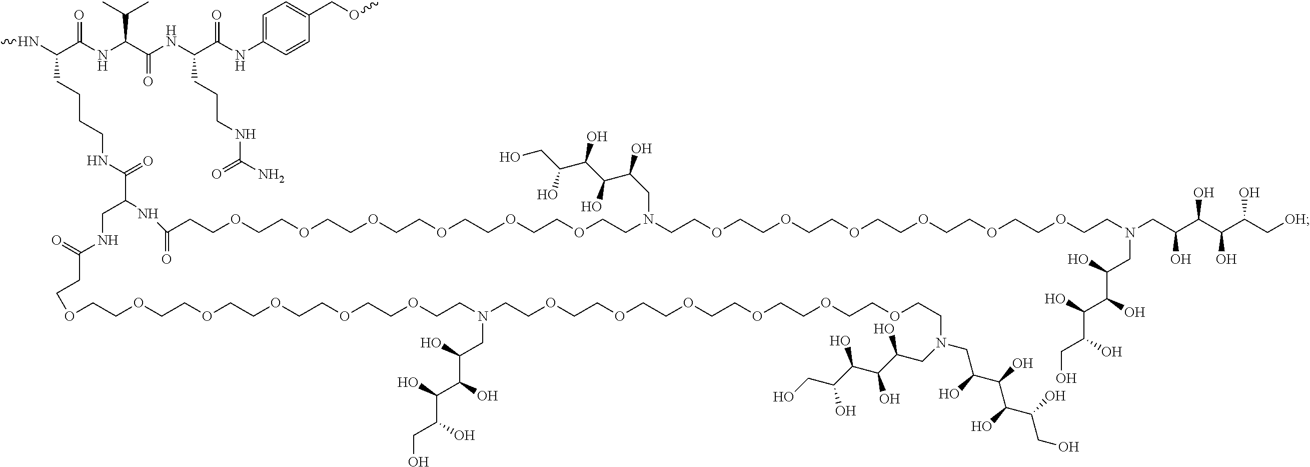

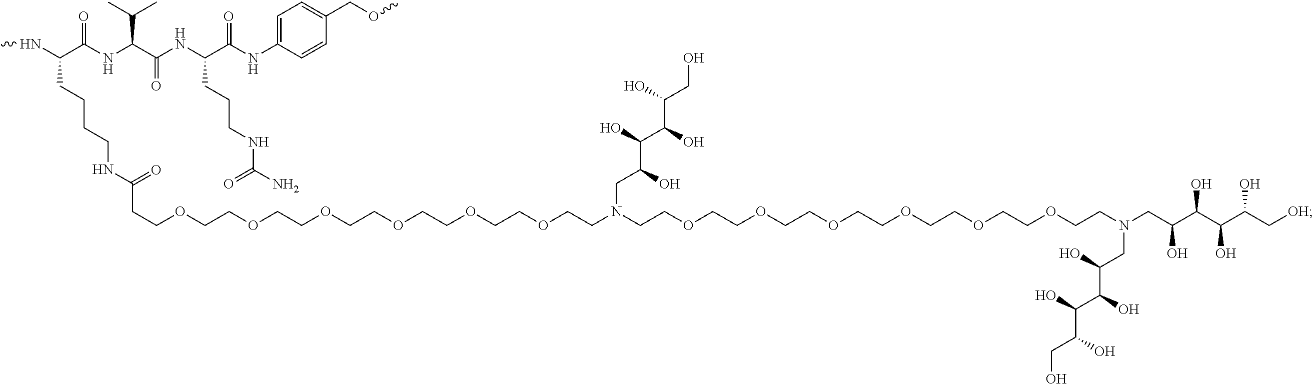

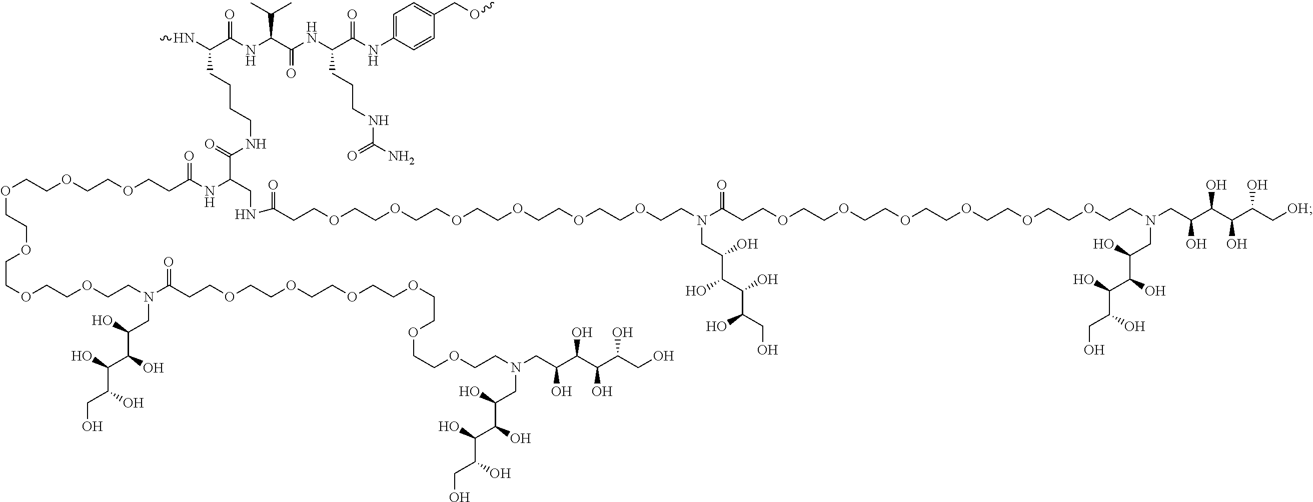

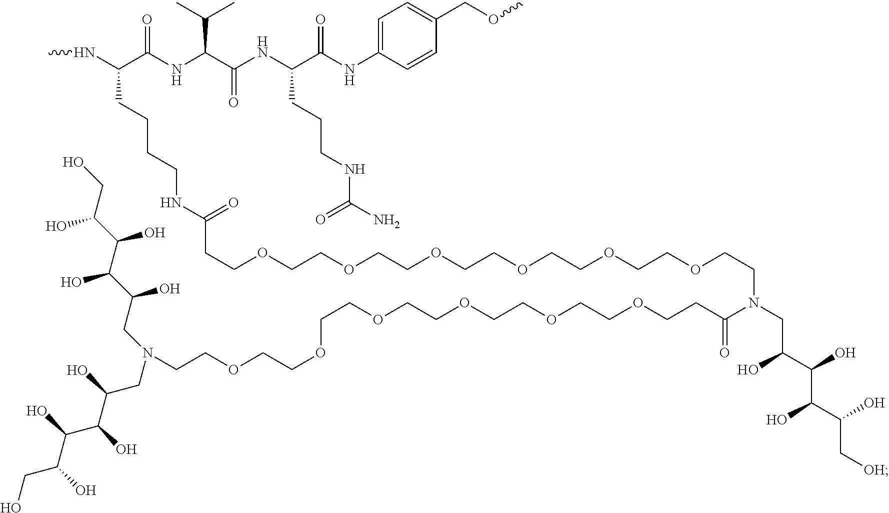

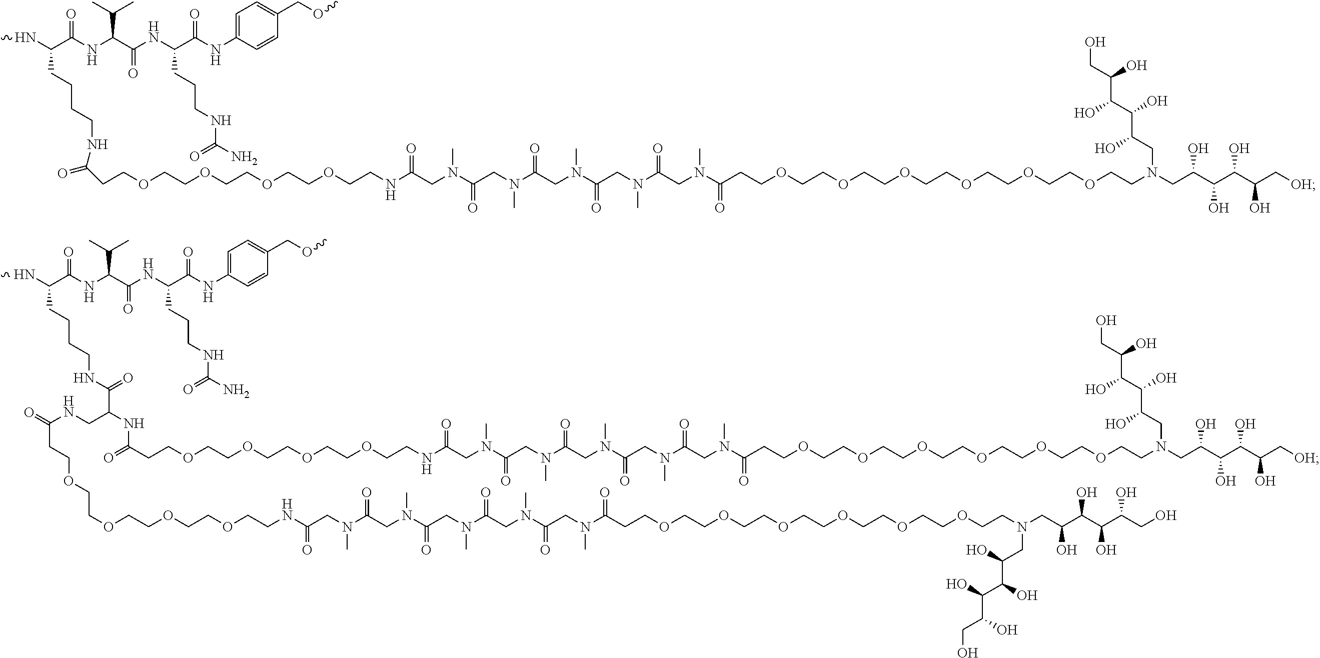

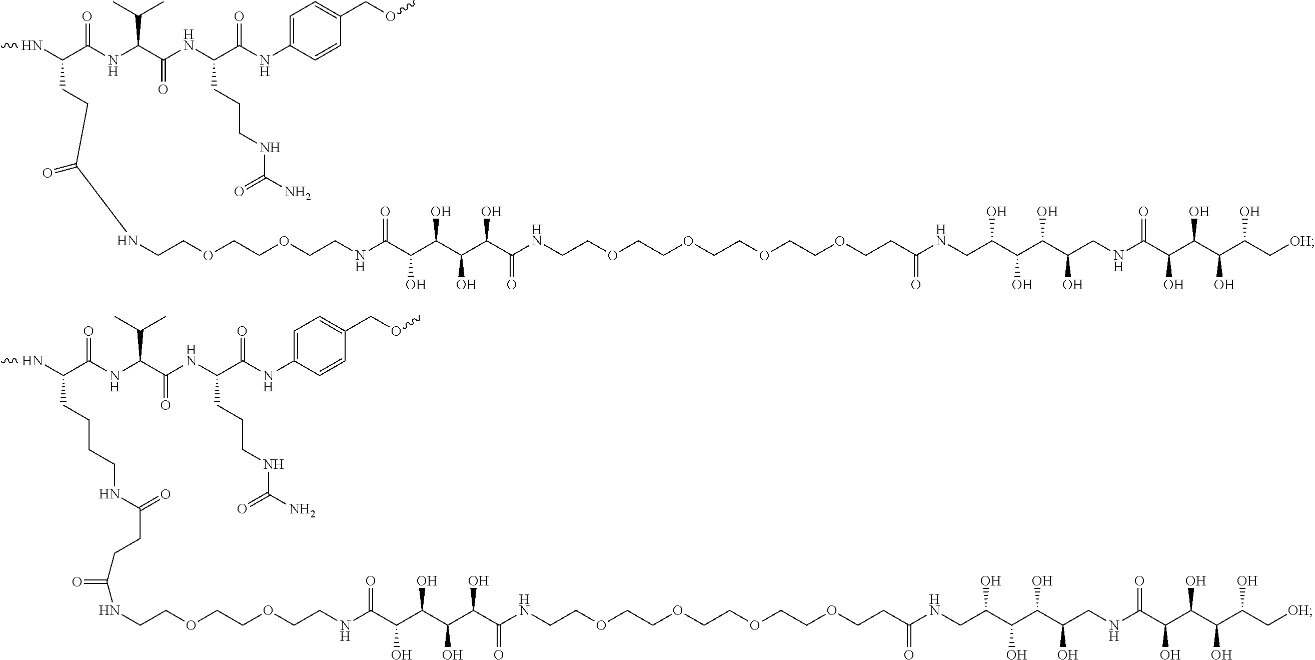

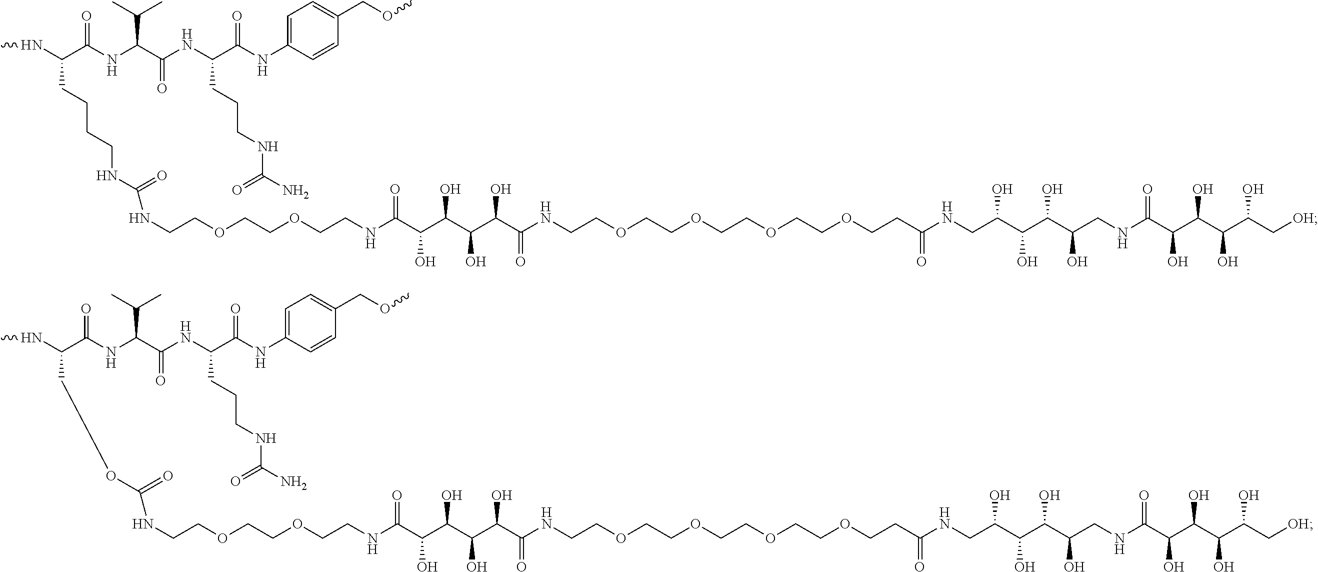

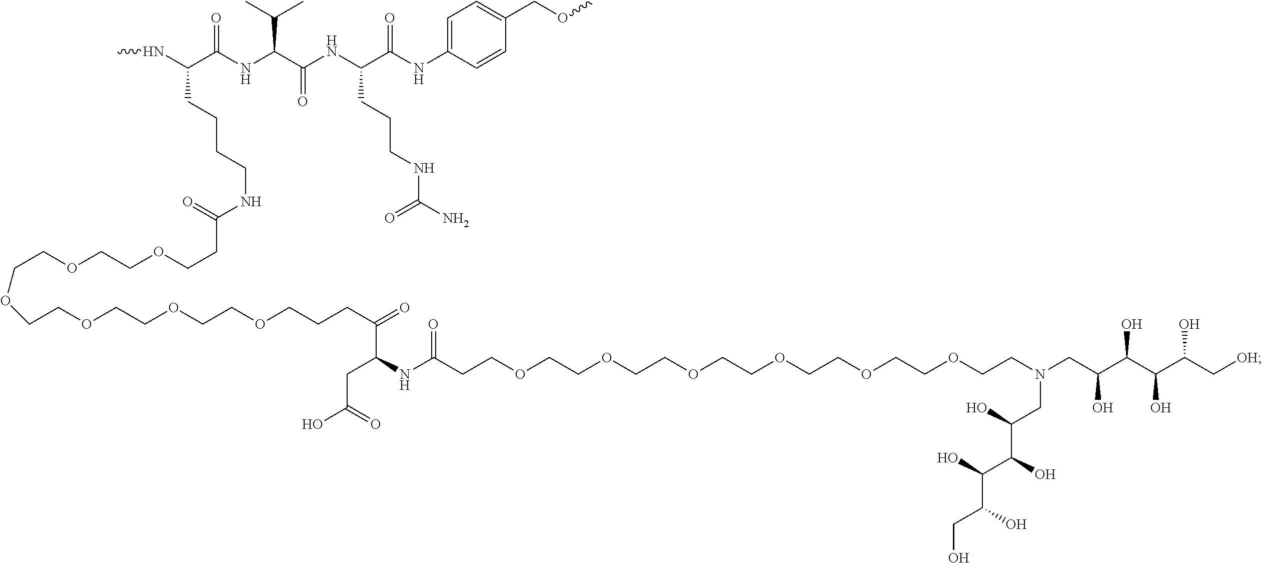

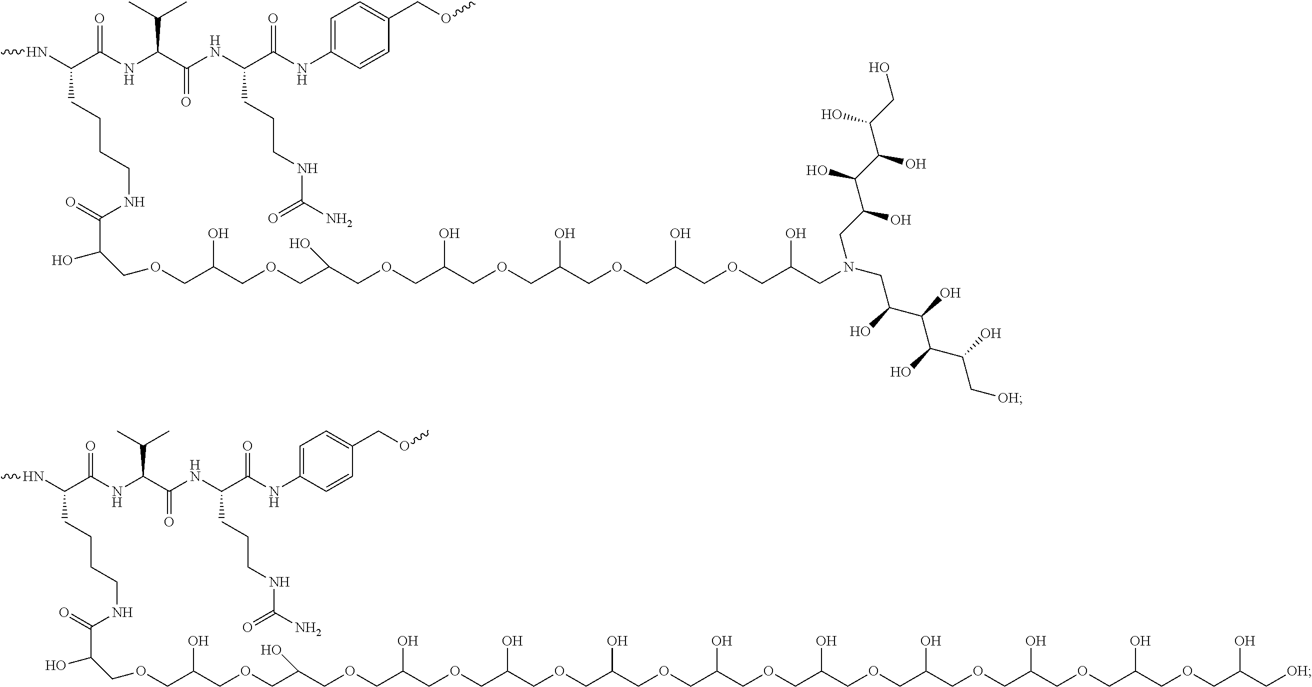

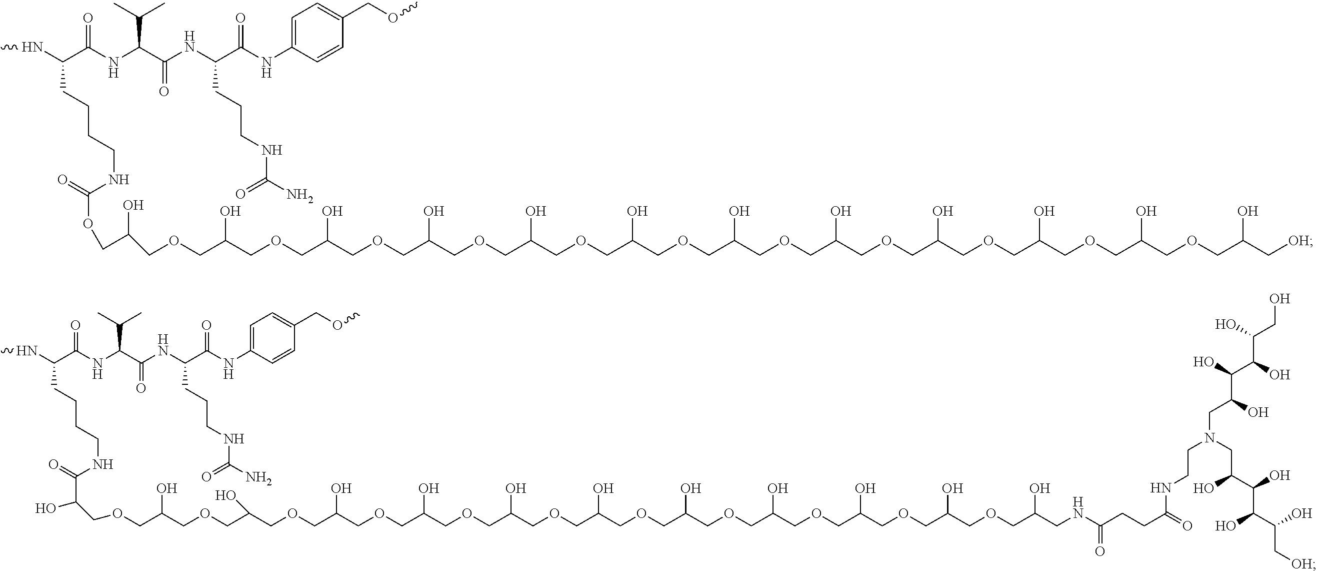

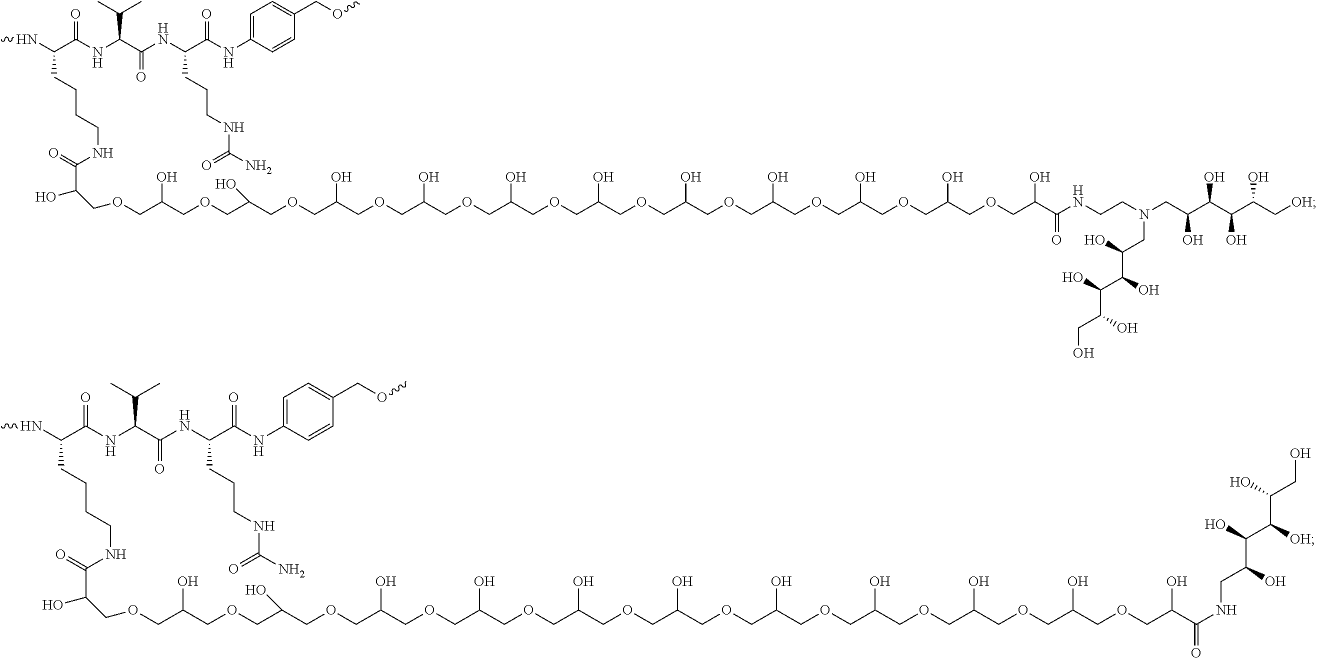

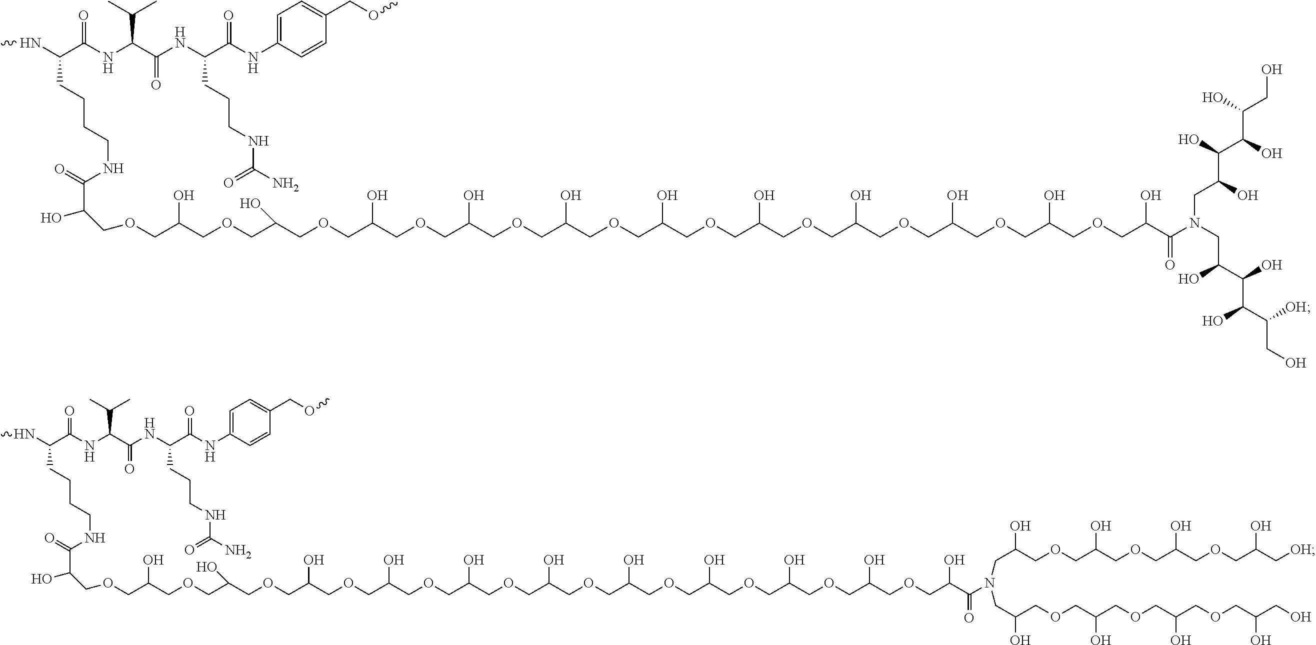

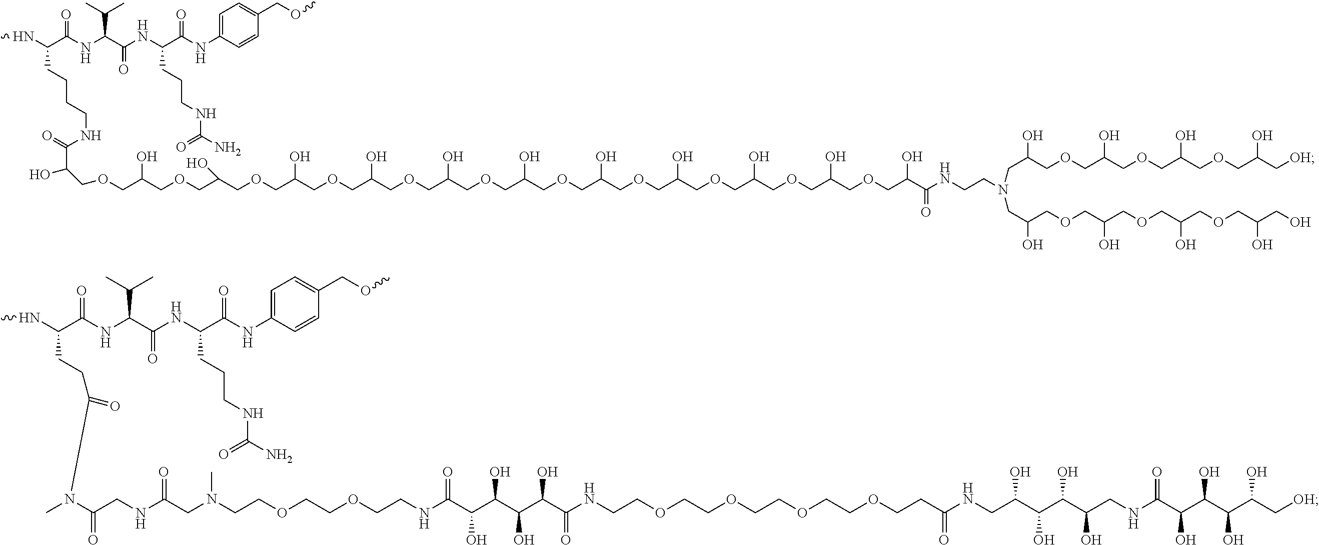

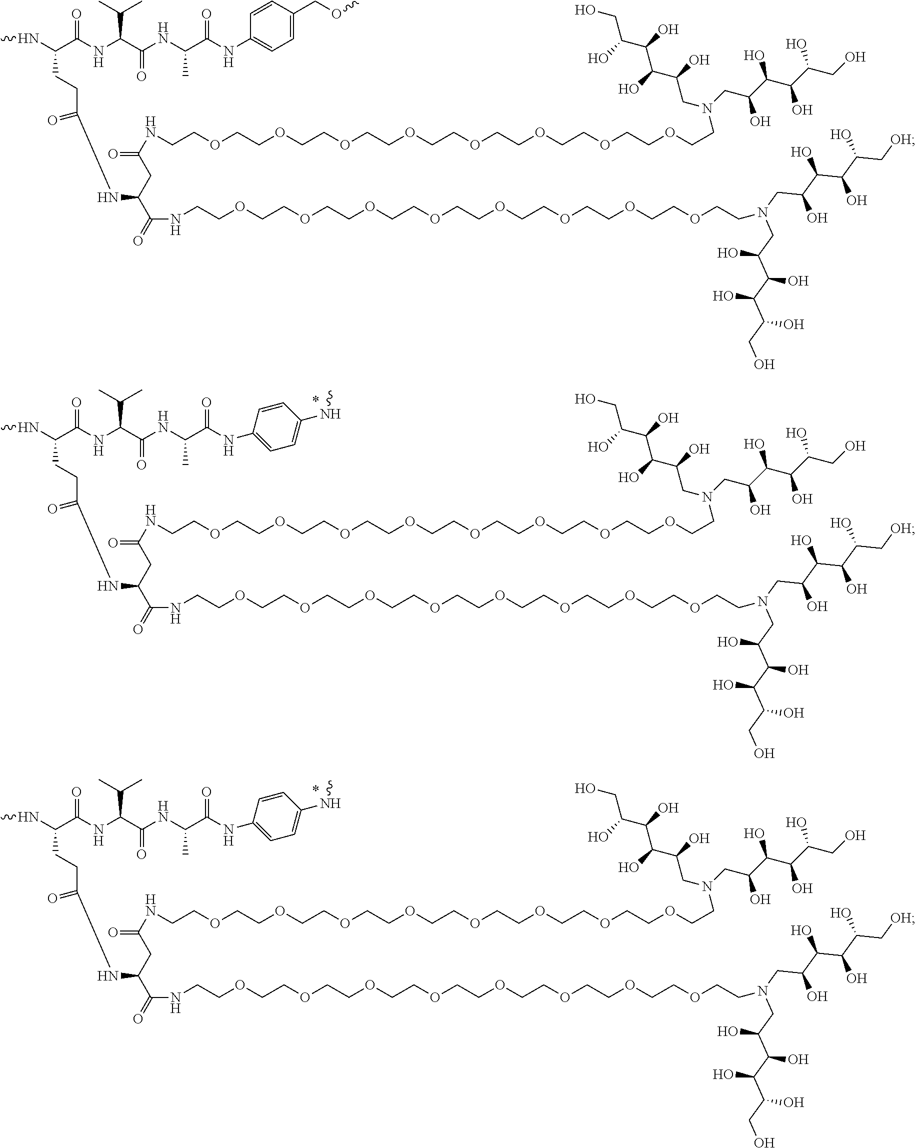

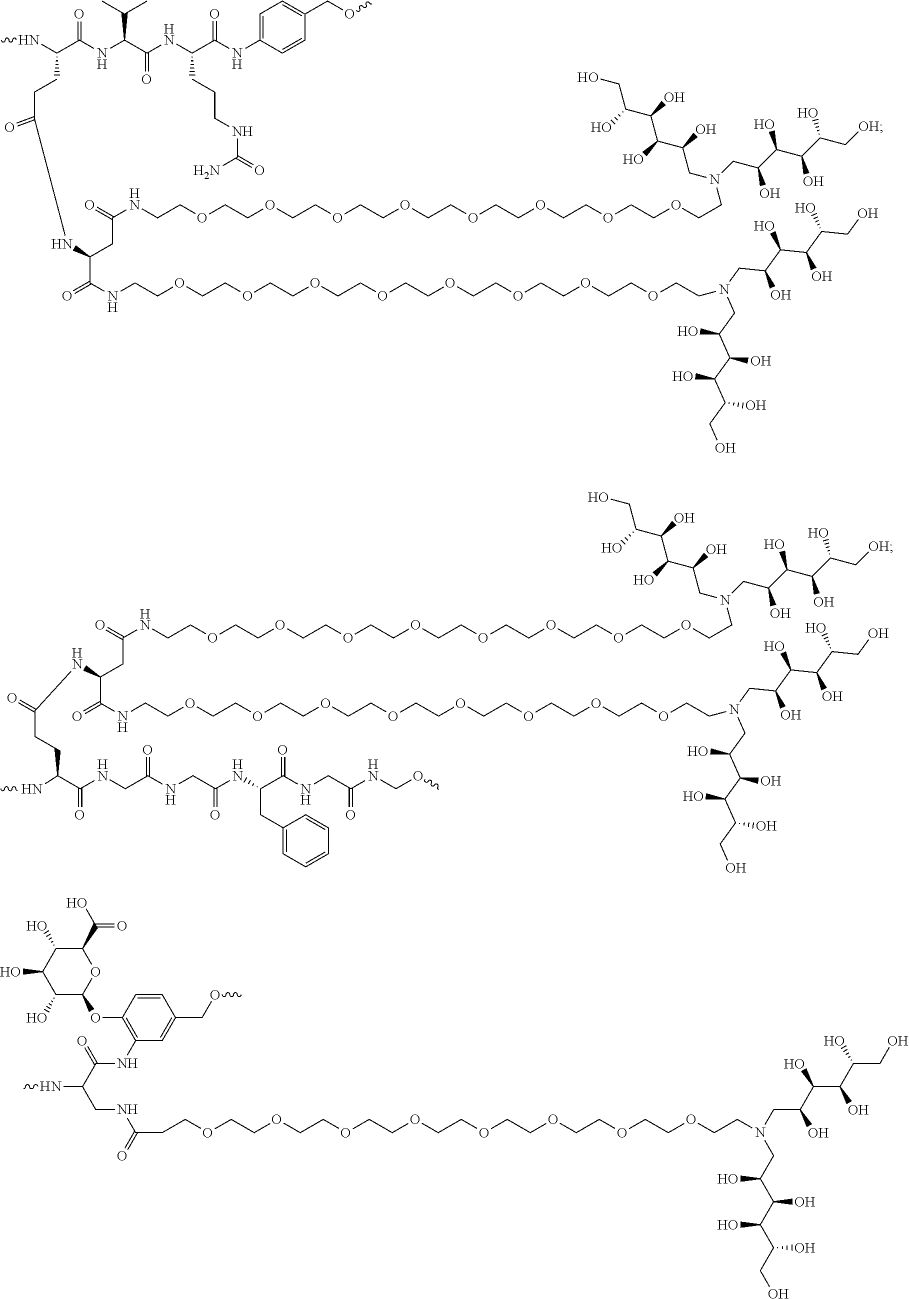

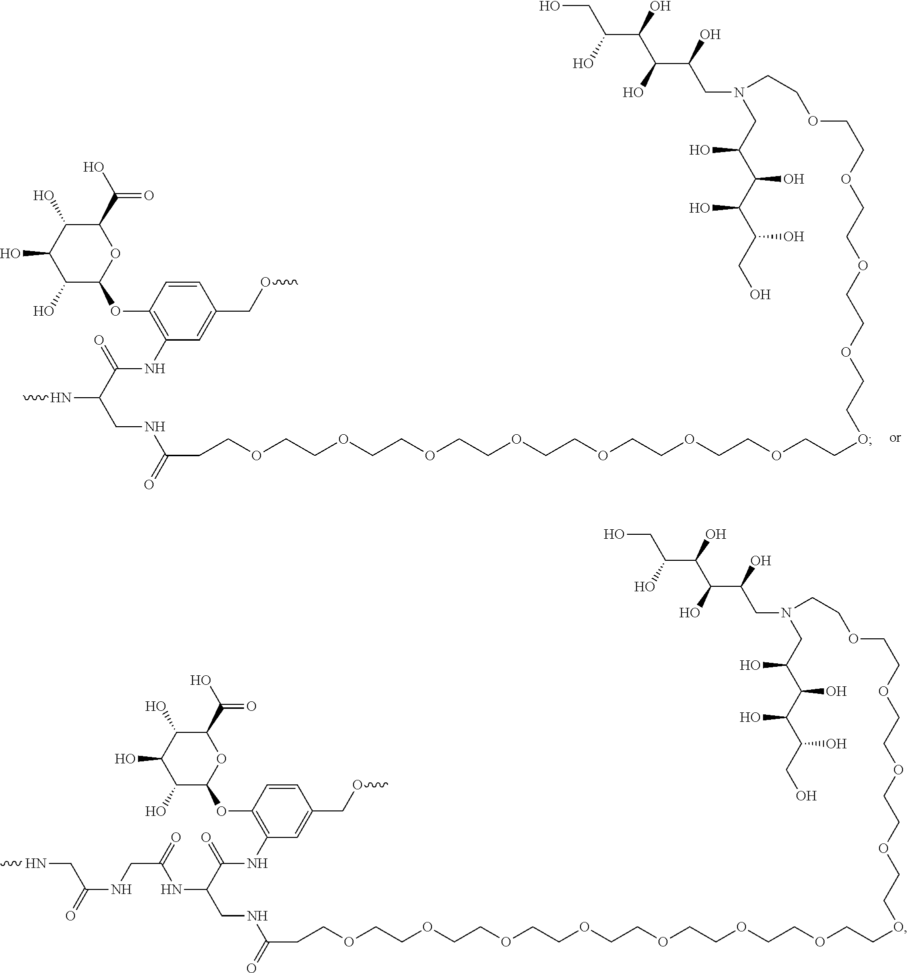

wherein the wavy line [symbol] on the oxygen group or the *-amino group indicates the attachment site to at least one of the drug units or for a linking group attached to the at least one of the drug units; and the wavy line [symbol] on the amino group indicates an attachment site for the stretcher group or the amino acid unit or, prior to attachment, indicates H.

Embodiment 101. The linker compound of any one of Embodiments 1 to 100, wherein the enzyme-cleavable group is joined to the Stretch group by a non-peptidic linking group.

Embodiment 102. The linker compound of Embodiment 101, wherein the non-peptidic linking group is selected from optionally-substituted C₁-C₁₀ alkylene, optionally-substituted C₂-C₁₀ alkenylene, optionally-substituted C₂-C₁₀ alkynylene, or optionally-substituted polyethylene glycol.

Embodiment 103. The linker compound of any one of Embodiments 1-102, comprising the stretcher group attached to the enzyme-cleavable group.

Embodiment 104. The linker compound of Embodiment 103, wherein the stretcher group is selected from the following:

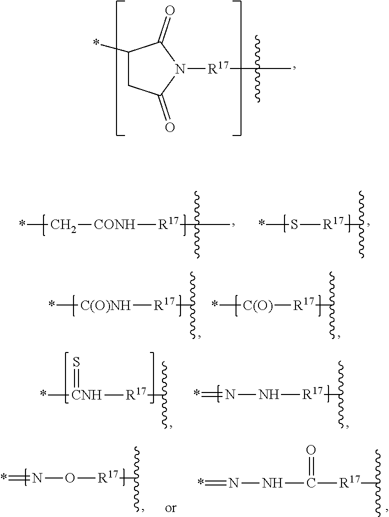

wherein R¹⁷ is —C₁-C₁₀ alkylene-, —C₁-C₁₀ heteroalkylene-, —C₃-C₈ carbocyclo-, —O—(C₁-C₈ alkylene)-, —(CH₂—O—CH₂)_{b}—C₁-C₈alkylene- (where b is 1 to 26), —C₁-C₈ alkylene-(CH₂—O—CH₂)_{b}— (where b is 1 to 26), —C₁-C₈ alkylene-(CH₂—O—CH₂)_{b}—C₁-C₈ alkylene- (where b is 1 to 26), -arylene-, —C₁-C₁₀ alkylene-arylene-, -arylene-C₁-C₁₀ alkylene-, —C₁-C₁₀ alkylene-(C₃-C₈ carbocyclo)-, —(C₃-C₈ carbocyclo)-C₁-C₁₀ alkylene-, —C₃-C₈ heterocyclo-, —C₁-C₁₀ alkylene-(C₃-C₈ heterocyclo)-, —(C₃-C₈ heterocyclo)-C₁-C₁₀ alkylene-, —C₁-C₁₀ alkylene-C(═O)—, —C₁-C₁₀alkylene-C(O)NH—C₁-C₈alkylene-[O—CH₂—CH₂]ₙ—C(O)— (where n is 1 to 26), C₁-C₁₀ heteroalkylene-C(═O)—, —C₁-C₈ alkylene-(CH₂—O—CH₂)_{b}—C(═O)— (where b is 1 to 26), —(CH₂—O—CH₂)_{b}—C₁-C₈ alkylene-C(═O)— (where b is 1 to 26), —C₁-C₈ alkylene-(CH₂—O—CH₂)_{b}—C₁-C₈ alkylene-C(═O)— (where b is 1 to 26), —C₃-C₈ carbocyclo-C(═O)—, —O—(C₁-C₈ alkyl)-C(═O)—, -arylene-C(═O)—, —C₁-C₁₀ alkylene-arylene-C(═O)—, -arylene-C₁-C₁₀ alkylene-C(═O)—, —C₁-C₁₀ alkylene-(C₃-C₈ carbocyclo)-C(═O)—, —(C₃-C₈ carbocyclo)-C₁-C₁₀ alkylene-C(═O)—, —C₃-C₈ heterocyclo-C(═O)—, —C₁-C₁₀ alkylene-(C₃-C₈ heterocyclo)-C(═O)—, —(C₃-C₈ heterocyclo)-C₁-C₁₀ alkylene-C(═O)—, —C₁-C₁₀ alkylene-NH—, —C₁-C₁₀ heteroalkylene-NH—, —C₁-C₈ alkylene-(CH₂—O—CH₂)_{b}—NH— (where b is 1 to 26), —(CH₂—O—CH₂)_{b}—C₁-C₈ alkylene-NH— (where b is 1 to 26), —C₁-C₈ alkylene-(CH₂—O—CH₂)_{b}—C₁-C₈ alkylene-NH— (where b is 1 to 26), —C₁-C₈ alkylene-(C(═O))—NH—(CH₂—O—CH₂)_{b}—C(═O)— (where b is 1 to 26), —C₁-C₈ alkylene-(C(═O))—NH—(CH₂—O—CH₂)_{b}—C₁-C₈ alkylene-C(═O)— (where b is 1 to 26), —C₁-C₈ alkylene-NH—(C(═O))—(CH₂—O—CH₂)_{b}—NH— (where b is 1 to 26), —C₁-C₈ alkylene-NH—(C(═O))—(CH₂—O—CH₂)_{b}—C₁-C₈ alkylene-NH— (where b is 1 to 26), —C₃-C₈ carbocyclo-NH—, —O—(C₁-C₈ alkyl)-NH—, -arylene-NH—, —C₁-C₁₀ alkylene-arylene-NH—, -arylene-C₁-C₁₀ alkylene-NH—, —C₁-C₁₀ alkylene-(C₃-C₈ carbocyclo)-NH—, —(C₃-C₈ carbocyclo)-C₁-C₁₀ alkylene-NH—, —C₃-C₈ heterocyclo-NH—, —C₁-C₁₀ alkylene-(C₃-C₈ heterocyclo)-NH—, —(C₃-C₈ heterocyclo)-C₁-C₁₀ alkylene-NH—, —C₁-C₁₀ alkylene-S—, C₁-C₁₀heteroalkylene-S—, —C₃-C₈ carbocyclo-S—, —O—(C₁-C₈ alkyl)-S—, -arylene-S—, —C₁-C₁₀ alkylene-arylene-S—, -arylene-C₁-C₁₀ alkylene-S—, —C₁-C₁₀ alkylene-(C₃-C₈ carbocyclo)-S—, —(C₃-C₈ carbocyclo)-C₁-C₁₀ alkylene-S—, —C₃-C₈ heterocyclo-S—, —C₁-C₁₀ alkylene-(C₃-C₈ heterocyclo)-S—, or —(C₃-C₈ heterocyclo)-C₁-C₁₀ alkylene-S—; or
wherein the stretcher group comprises maleimido(C₁-C₁₀alkylene-C(O)—, maleimido(CH₂OCH₂)ₚ₂(C₁-C₁₀alkyene)C(O)—, maleimido(C₁-C₁₀alkyene) (CH₂OCH₂)ₚ₂C(O)—, or a ring open form thereof, wherein p2 is from 1 to 26;
and wherein * is an attachment to the SLITRK6 binding agent, and the wavy line is an attachment to the enzyme-cleavable group.

Embodiment 105. The linker compound of Embodiment 104, wherein the stretcher group is selected from the following:

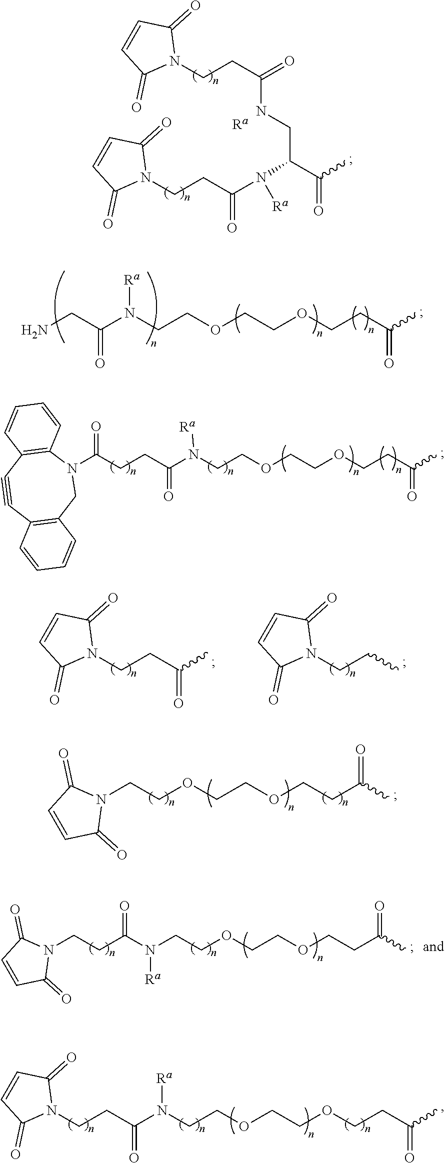

or a stereoisomer thereof, wherein each R^{a} is independently H or C₁₋₆ alkyl, each n is independently 0-12, and the wavy line [symbol] indicates an attachment site of the stretcher group to the amino acid unit, and the attachment site for targeting unit is on a maleimide, primary amine or alkyne functional group.

Embodiment 106. The linker compound of Embodiment 104, wherein the stretcher group is selected from the following:

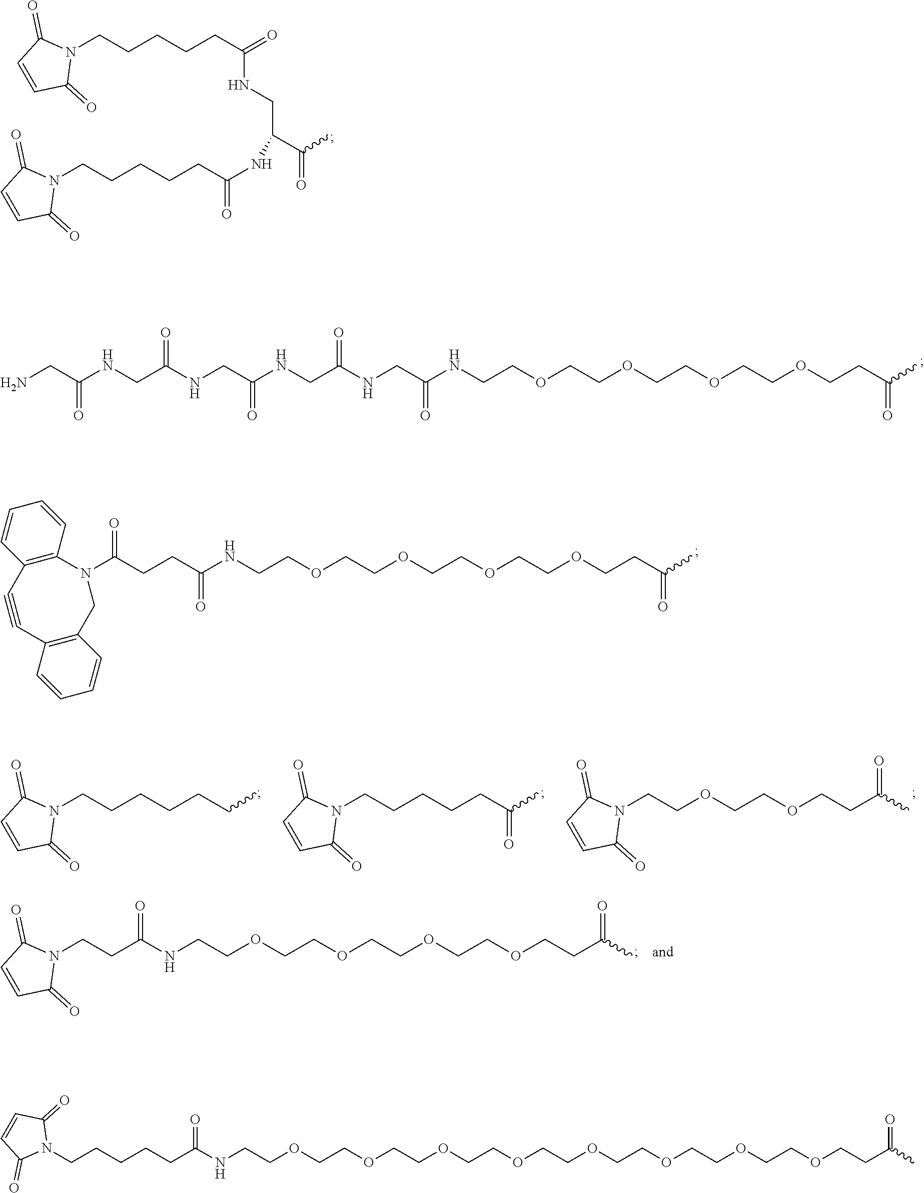

wherein the wavy line [symbol] indicates an attachment site of the stretcher group to the enzyme-cleavable group, and the attachment site to the SLITRK6 binding agent is on the maleimide, primary amine or alkyne functional group.

Embodiment 107. The linker compound of any one of Embodiments 1-9, having one of the following structures:

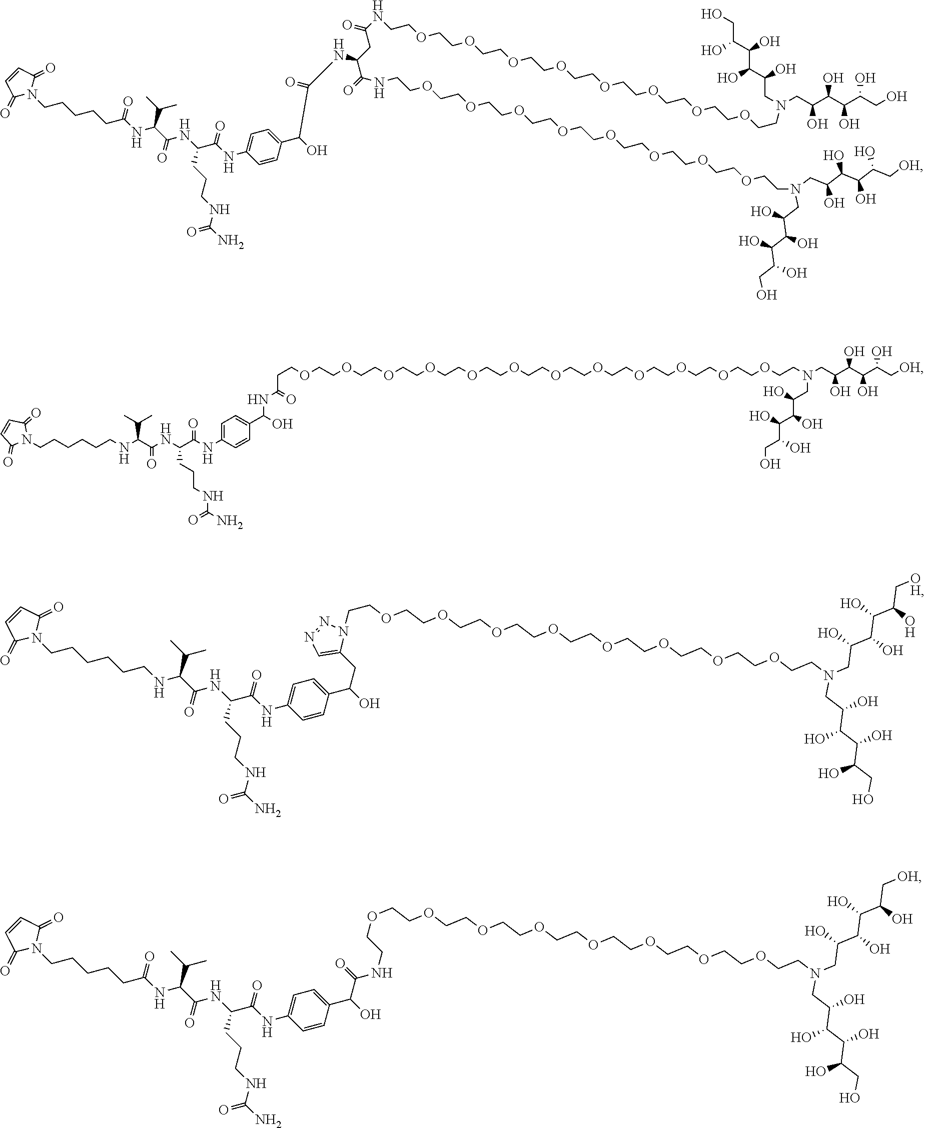

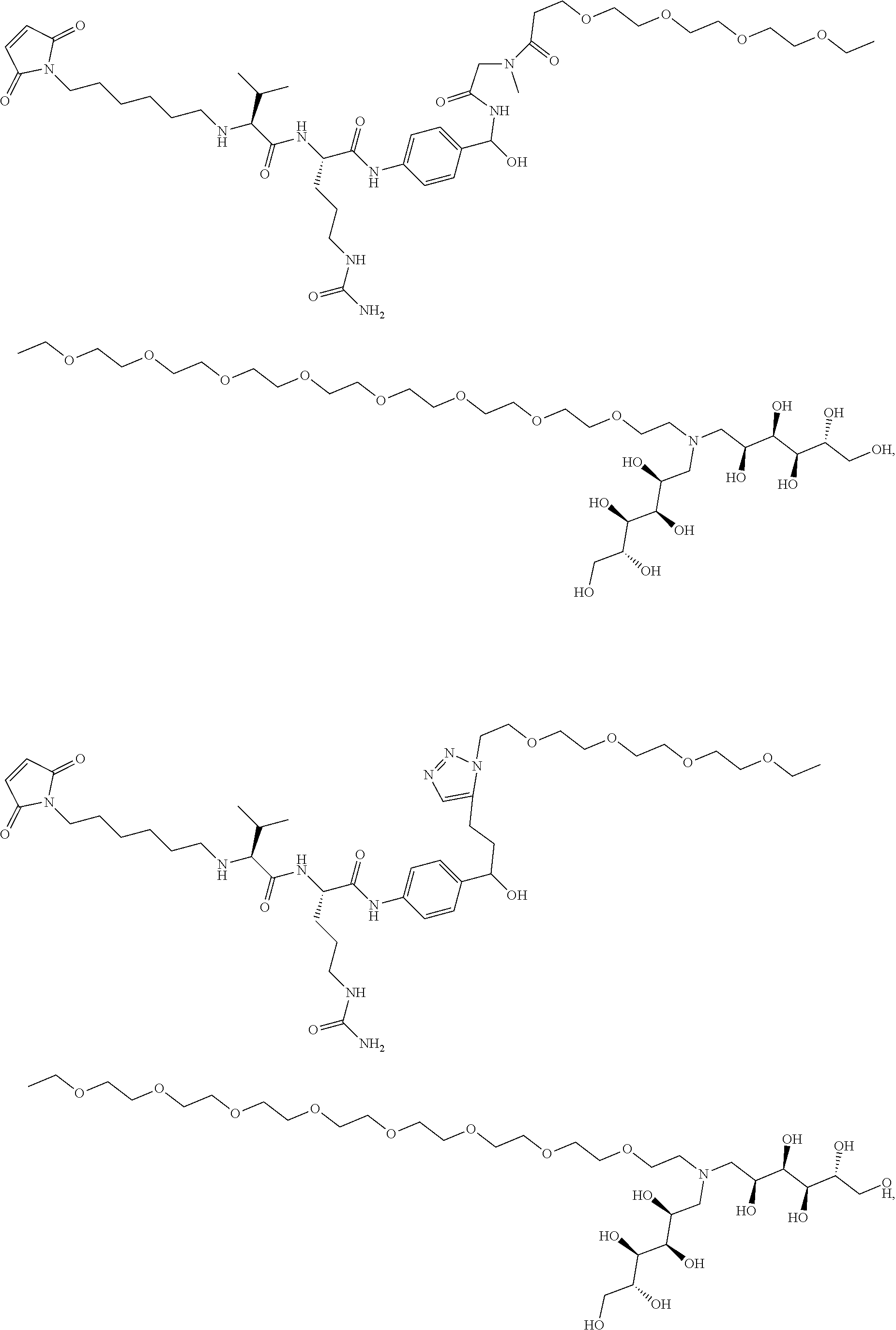

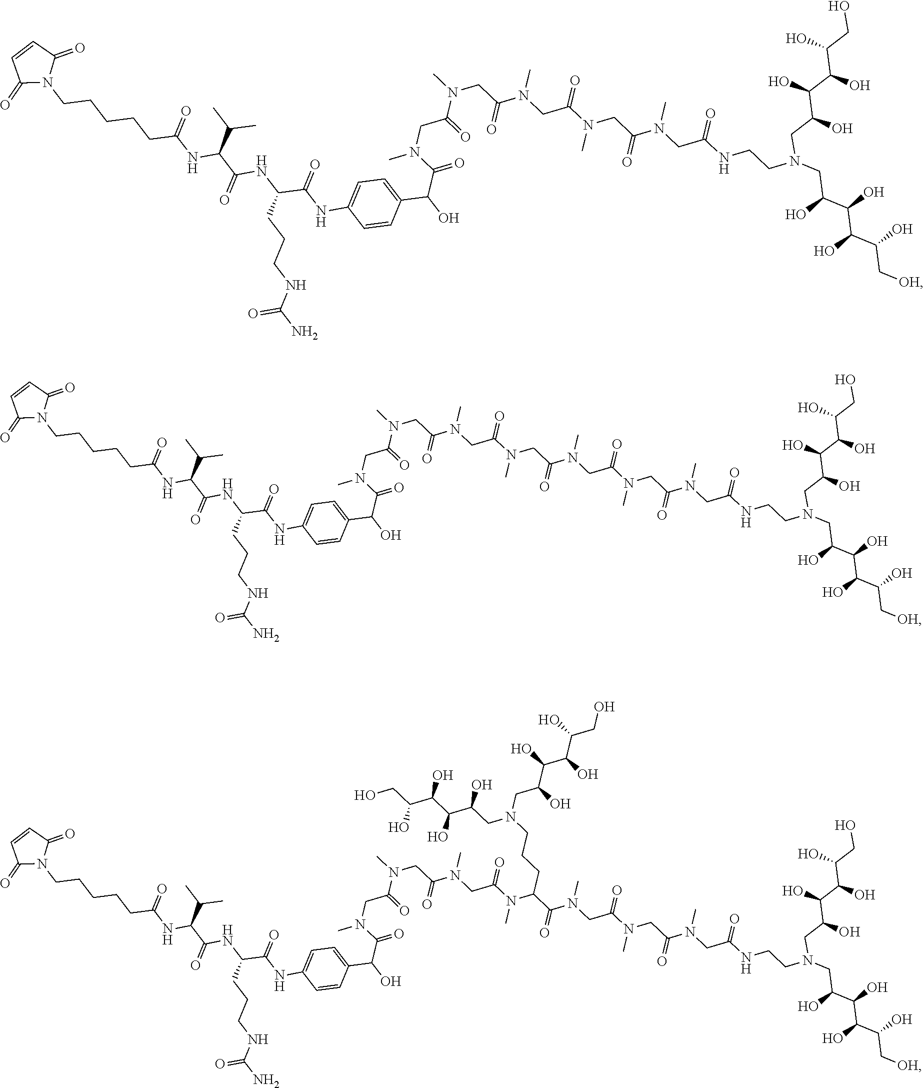

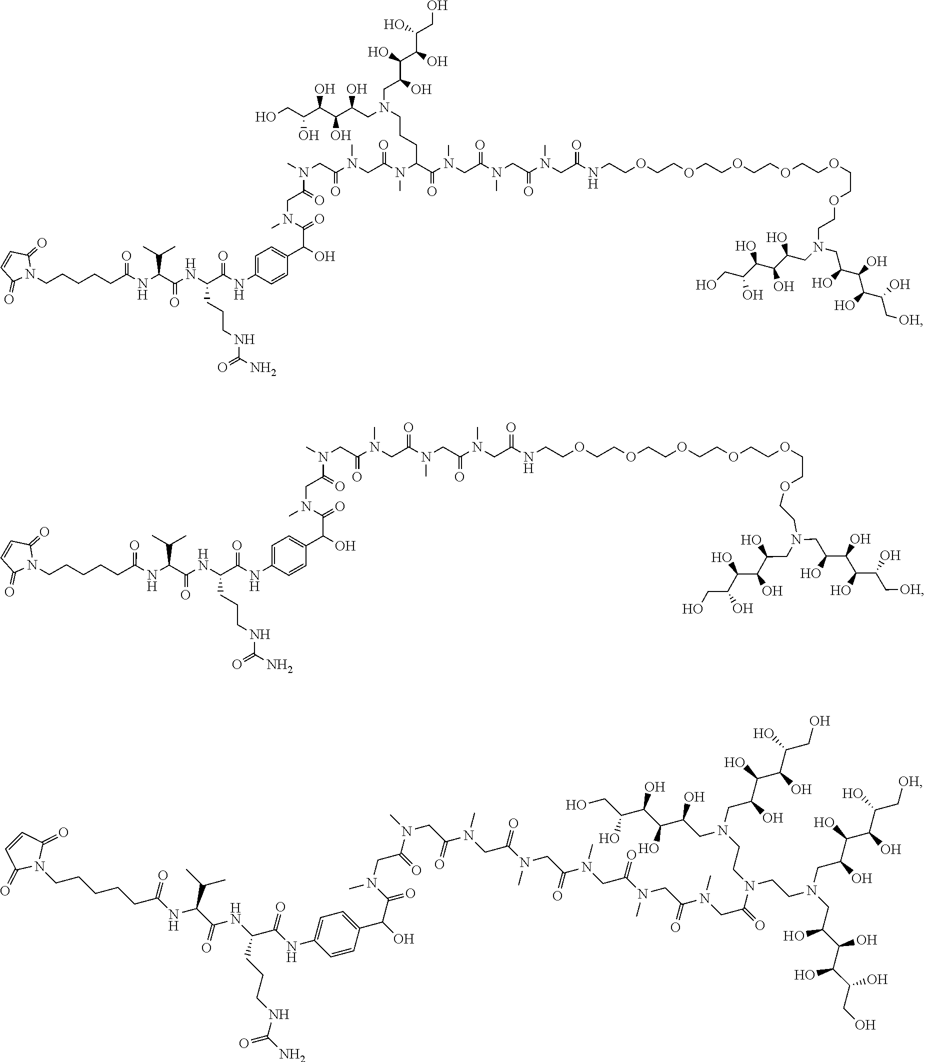

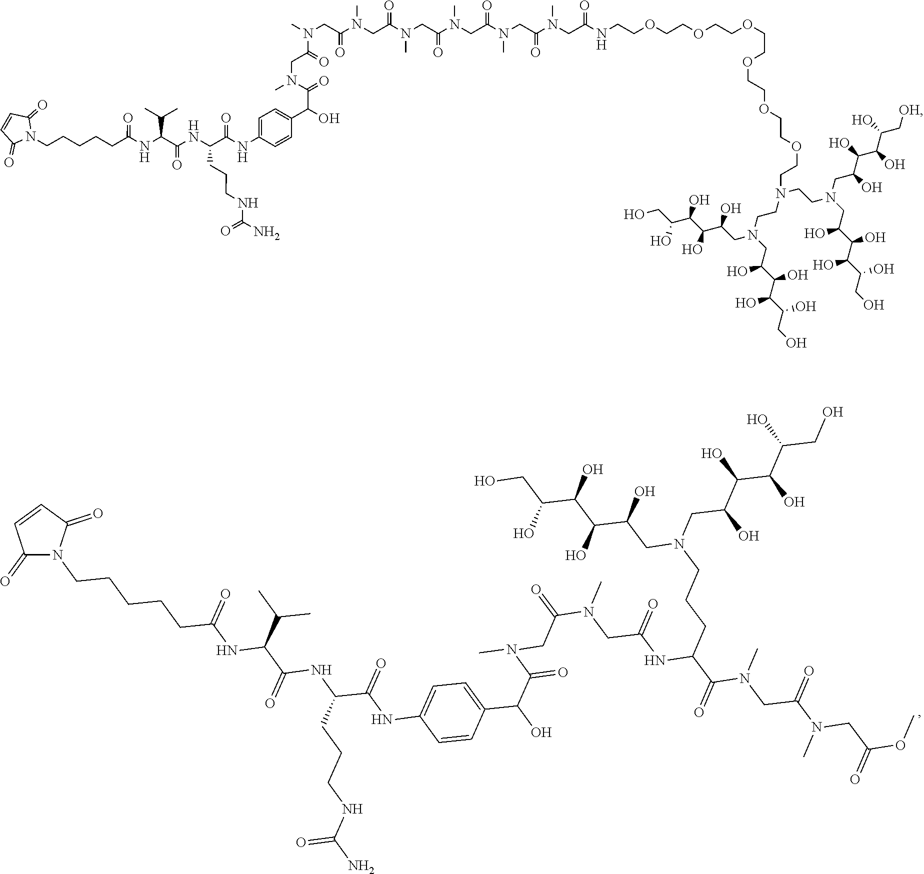

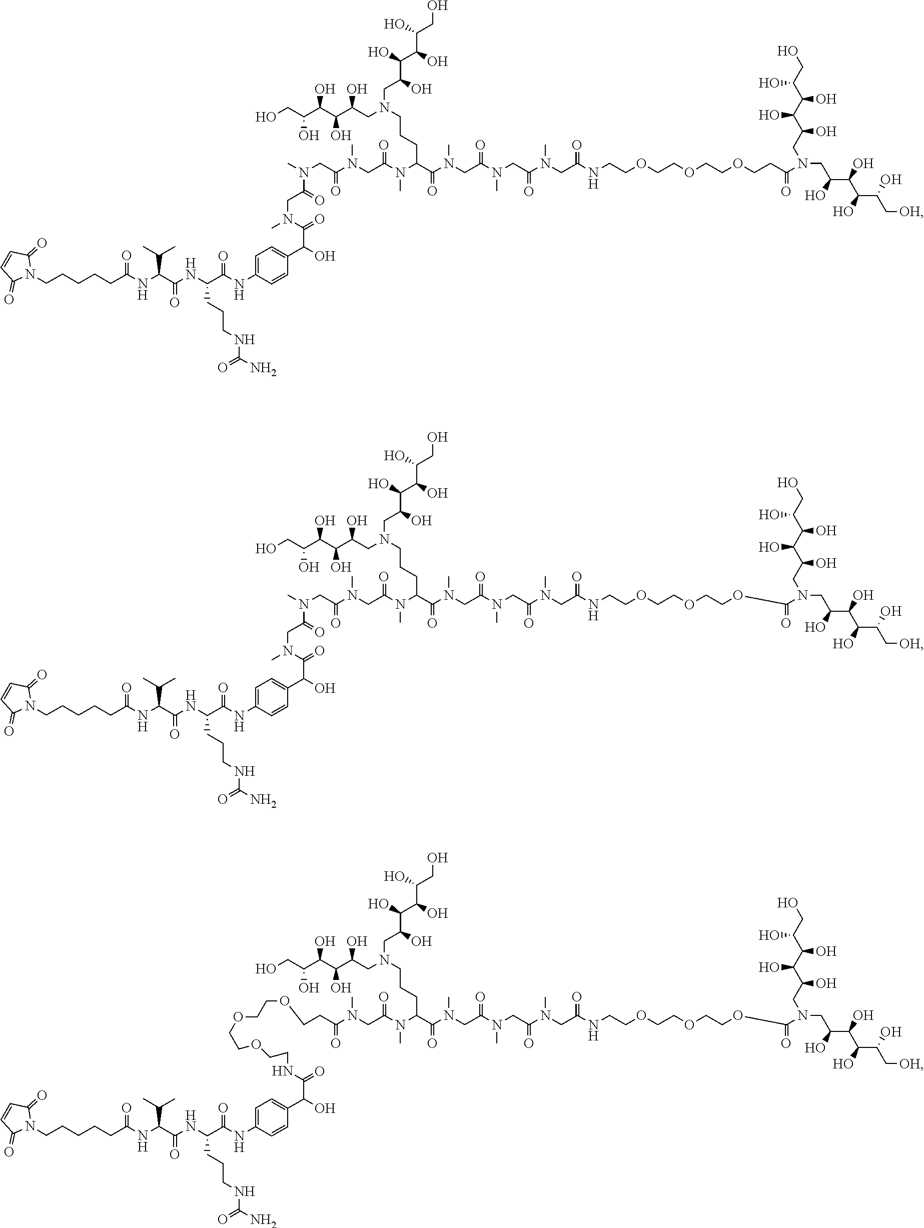

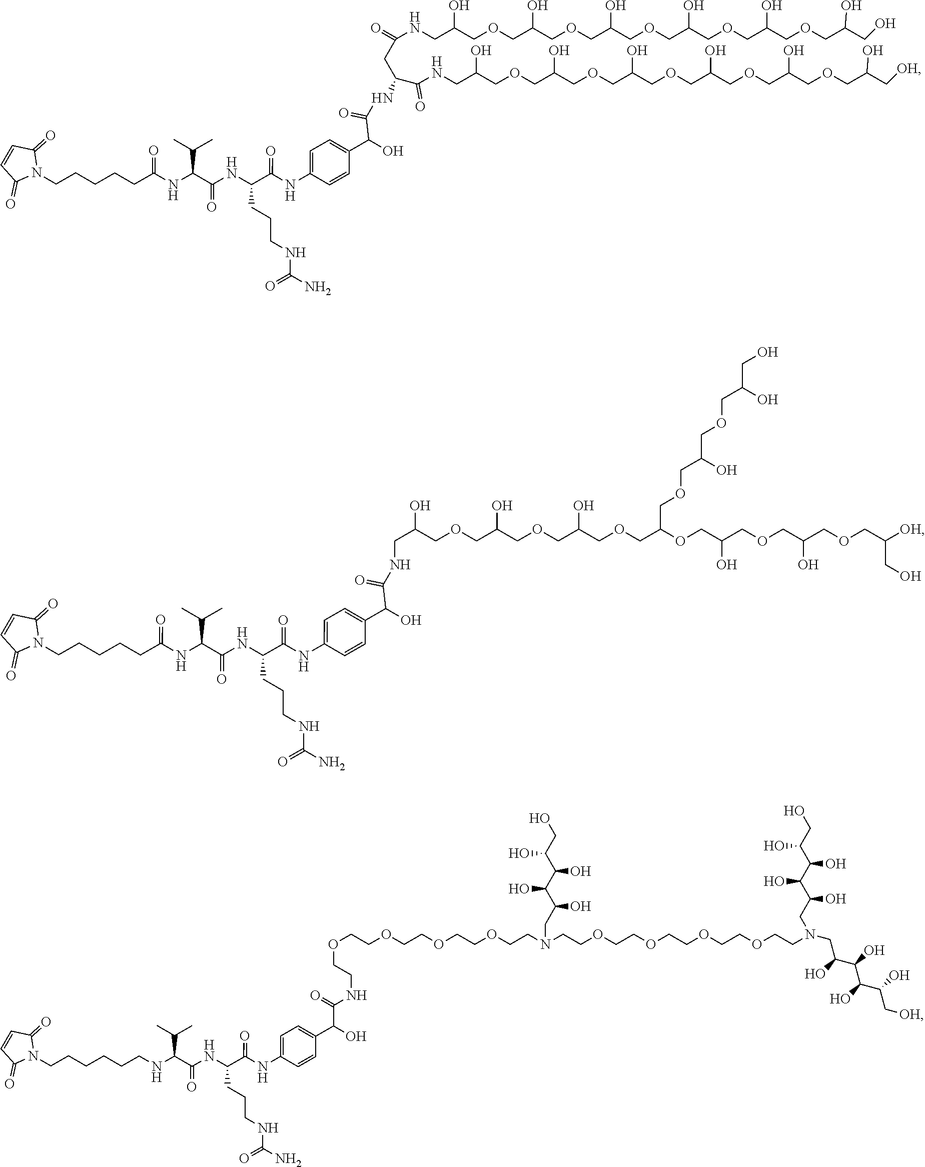

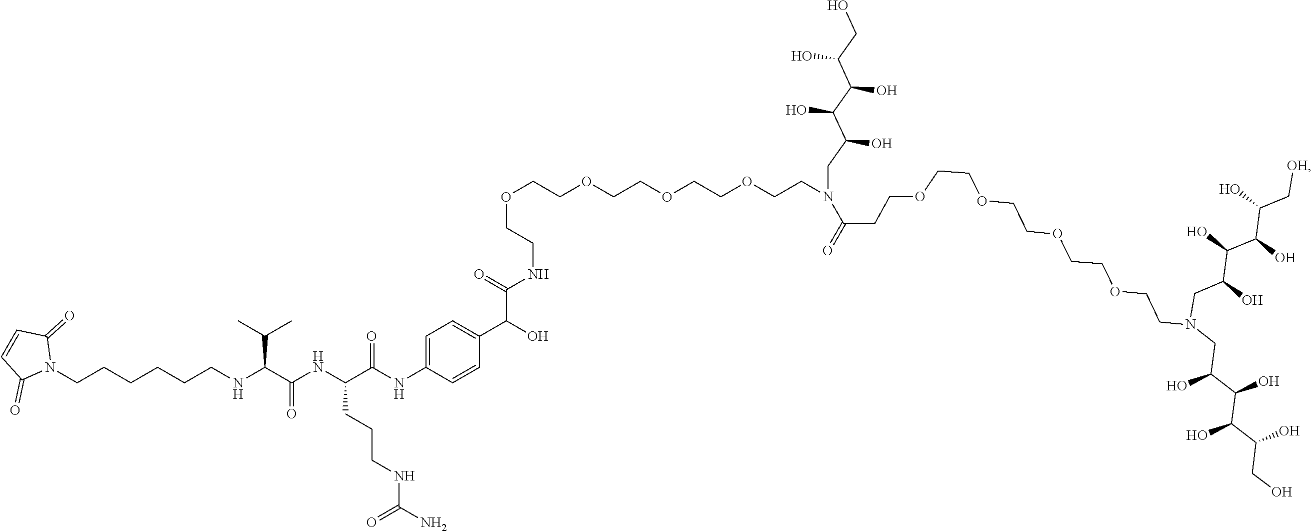 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)cc1]

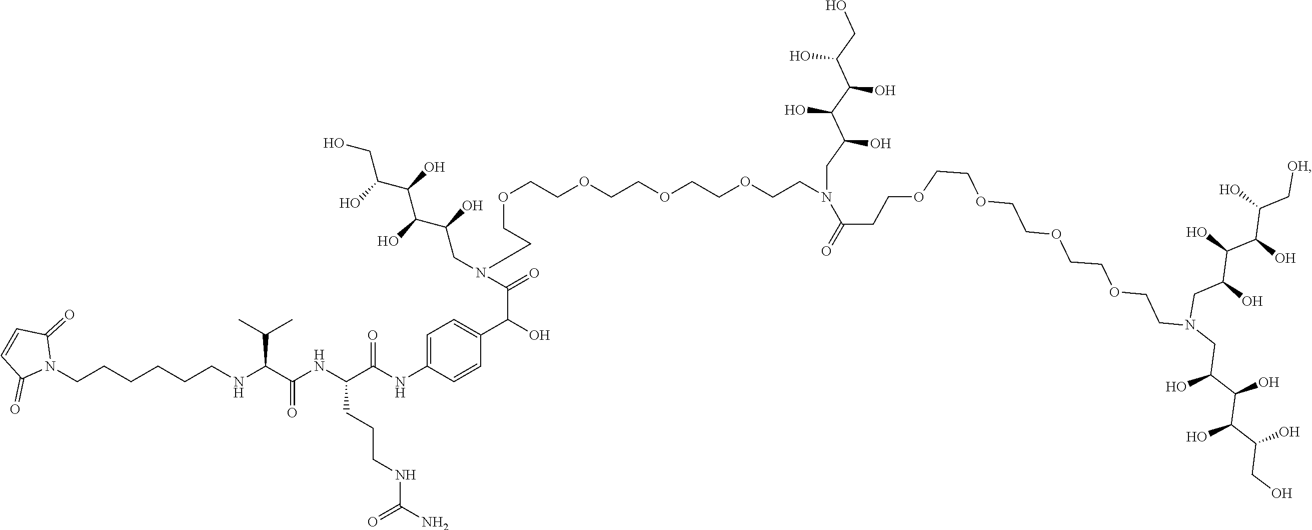 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCN(C[C@H](O)[C@@H](O)[C@H](O)[C@H](O)CO)C(=O)CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

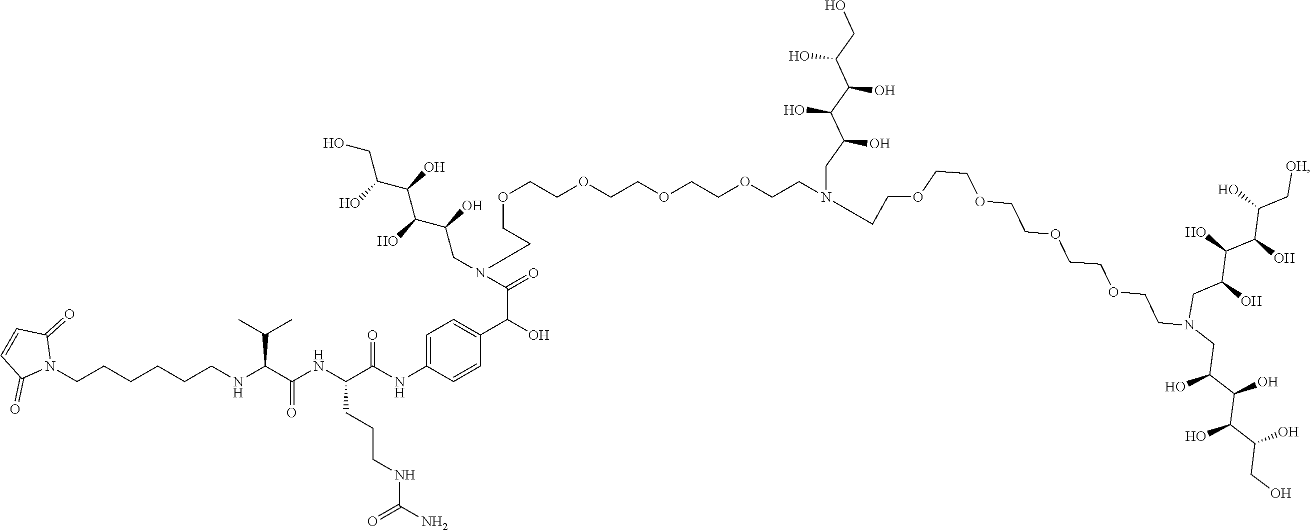 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCN(CCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

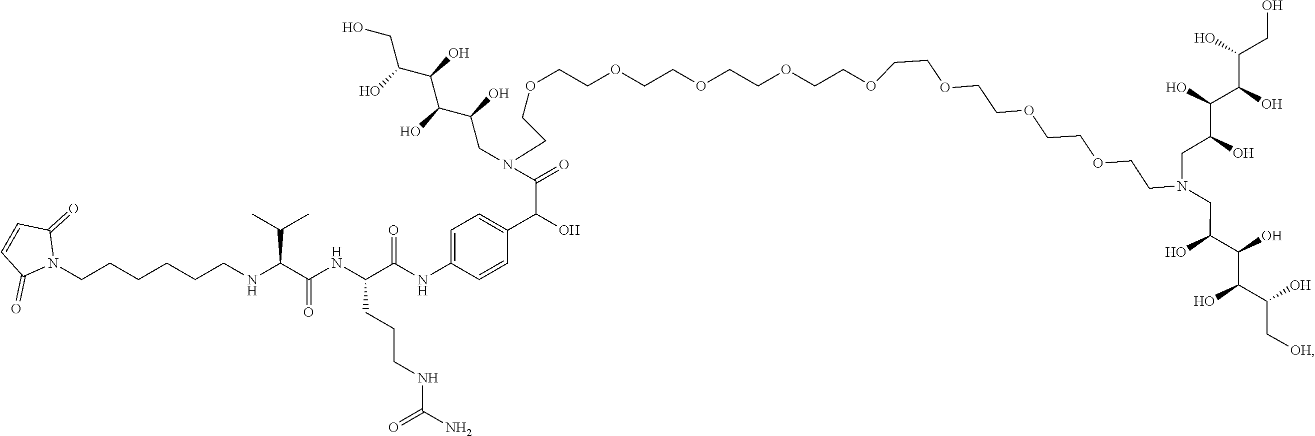 [SMILES: CC(C)[C@H](NCCCCCCN1C(=O)C=CC1=O)C(=O)N[C@@H](CCCNC(N)=O)C(=O)Nc1ccc(C(O)C(=O)N(CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C[C@H](O)[C@@H](O)[C@H](O)[C@H](O)CO)cc1]

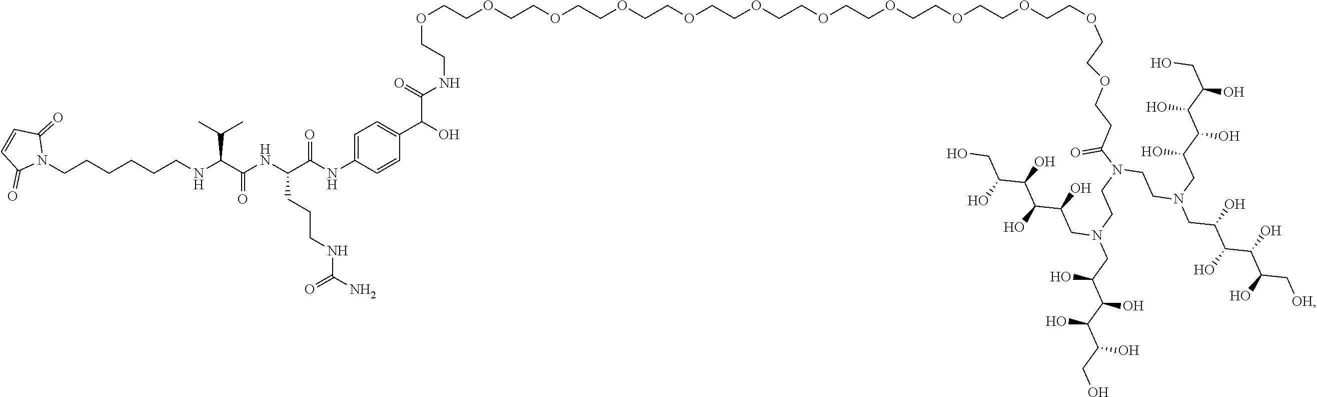 [SMILES: CC(C)[C@H](NCCCCCCN1C(=O)C=CC1=O)C(=O)N[C@@H](CCCNC(N)=O)C(=O)Nc1ccc(C(O)C(=O)NCCOCCOCCOCCOCCOCCOCCOCCOCCOCCOCCOCCOCCC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)cc1]

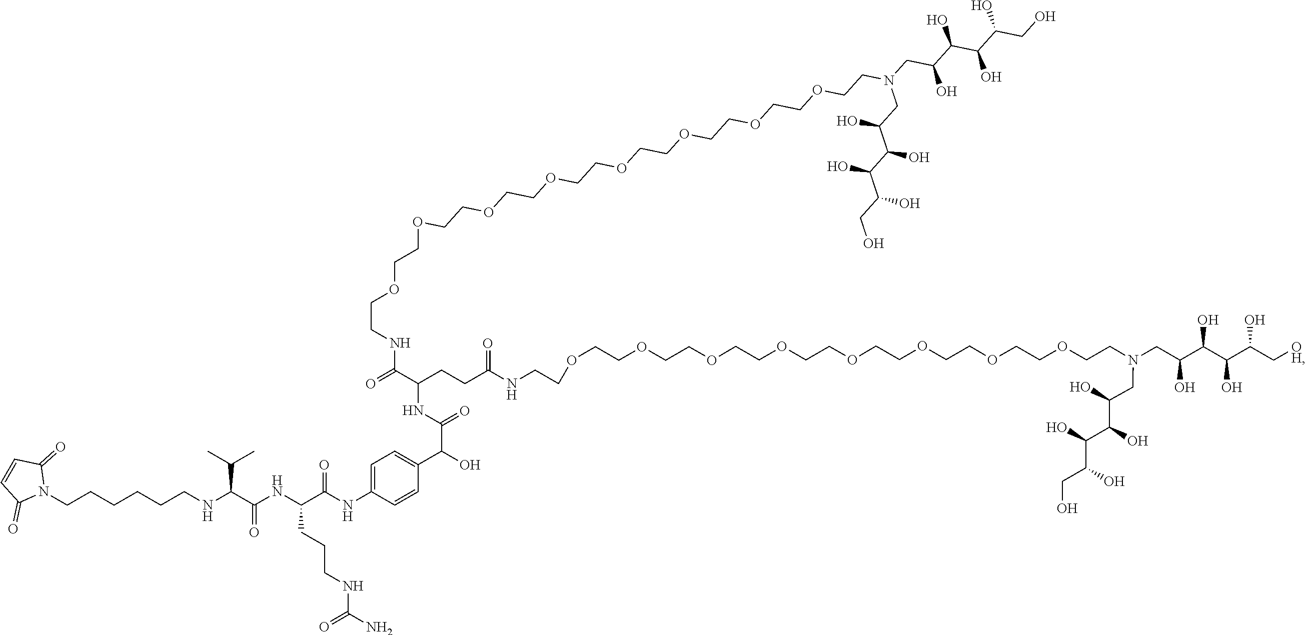 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

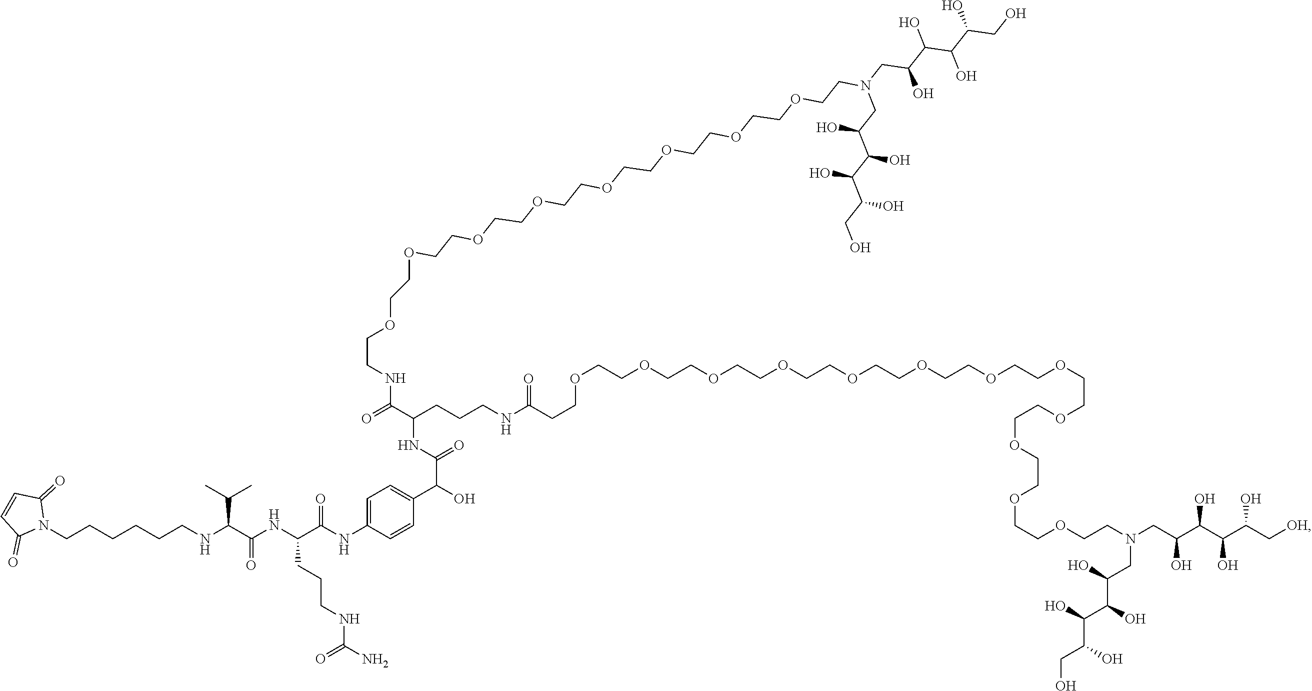 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)C(O)C(O)[C@H](O)CO)C[C@H](O)[C@@H](O)[C@H](O)[C@H](O)CO)cc1]

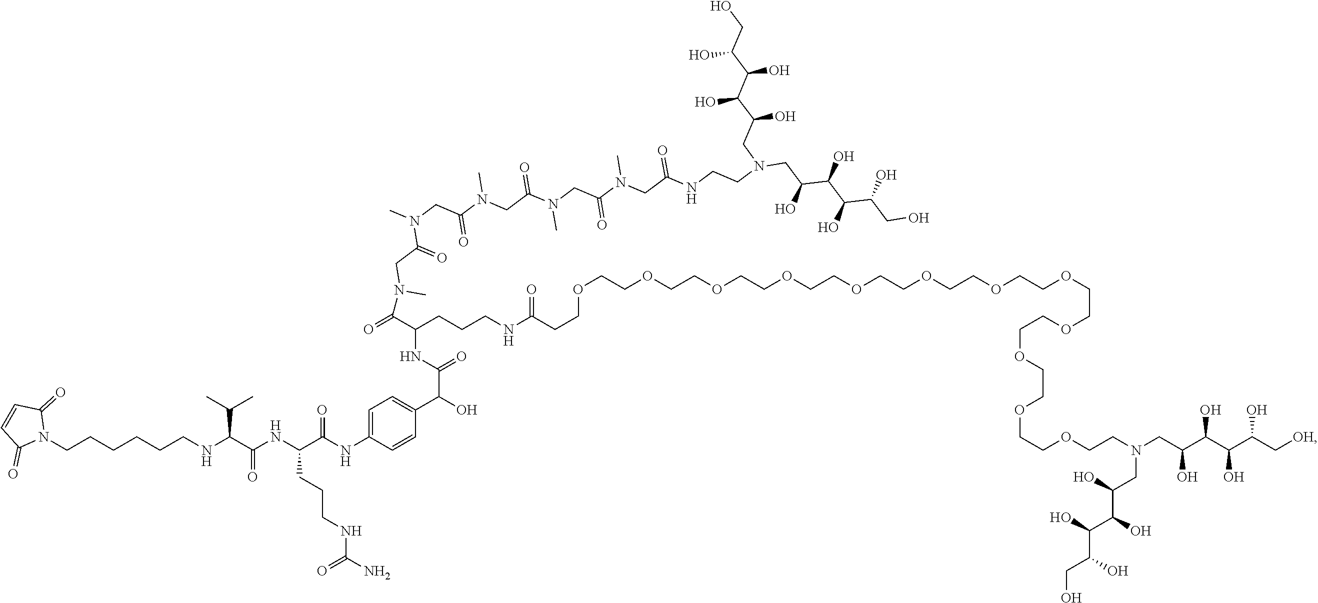 [SMILES: CC(C)[C@H](NCCCCCCN1C(=O)C=CC1=O)C(=O)N[C@@H](CCCNC(N)=O)C(=O)Nc1ccc(C(O)C(=O)NC(CCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

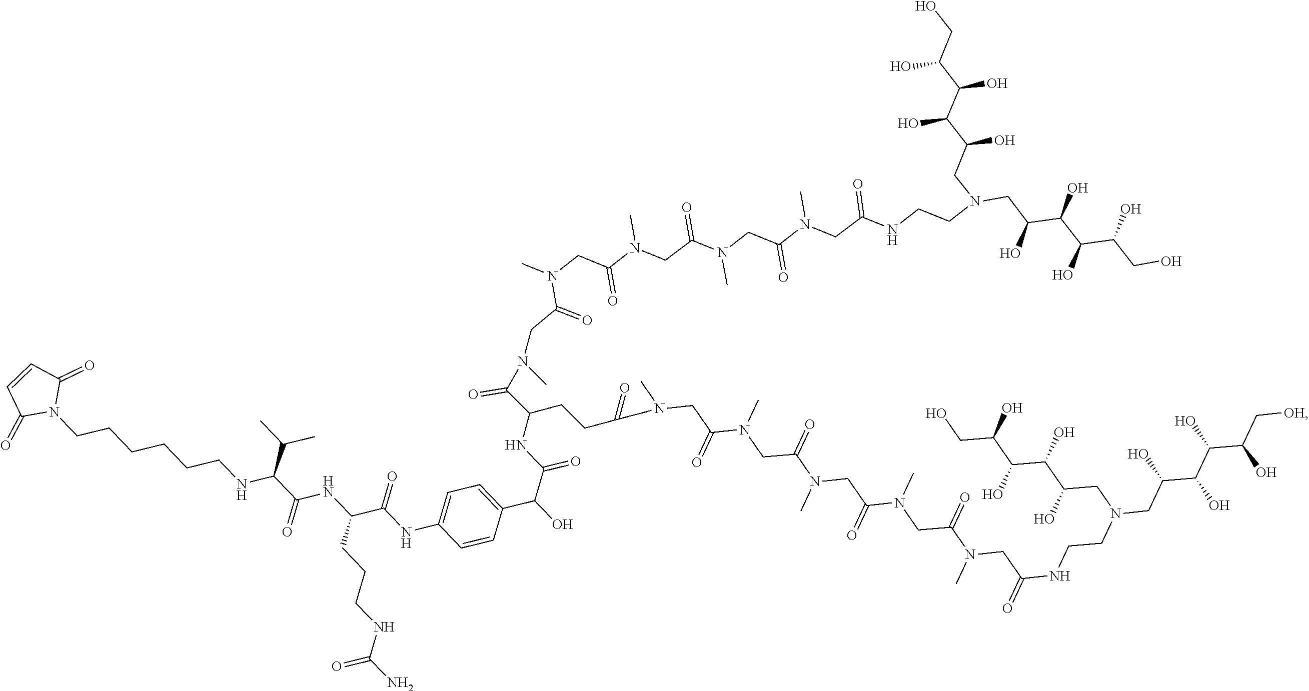 [SMILES: CC(C)[C@H](NCCCCCCN1C(=O)C=CC1=O)C(=O)N[C@@H](CCCNC(N)=O)C(=O)Nc1ccc(C(O)C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)cc1]

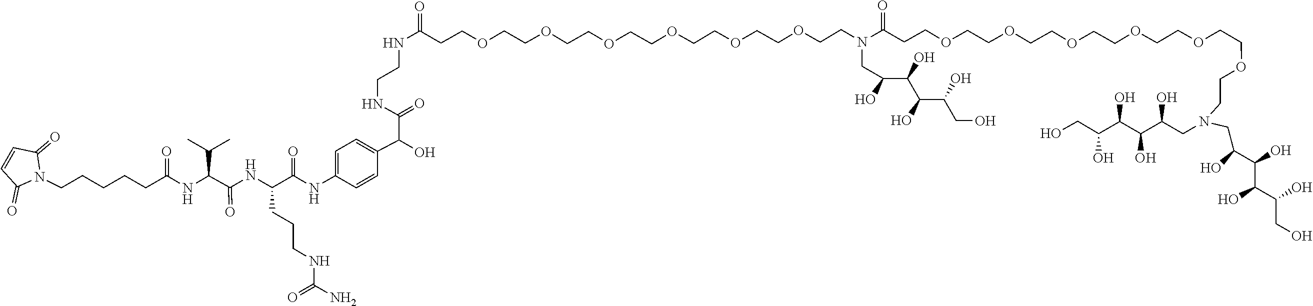 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCNC(=O)CCOCCOCCOCCOCCOCCOCCN(C[C@H](O)[C@@H](O)[C@H](O)[C@H](O)CO)C(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

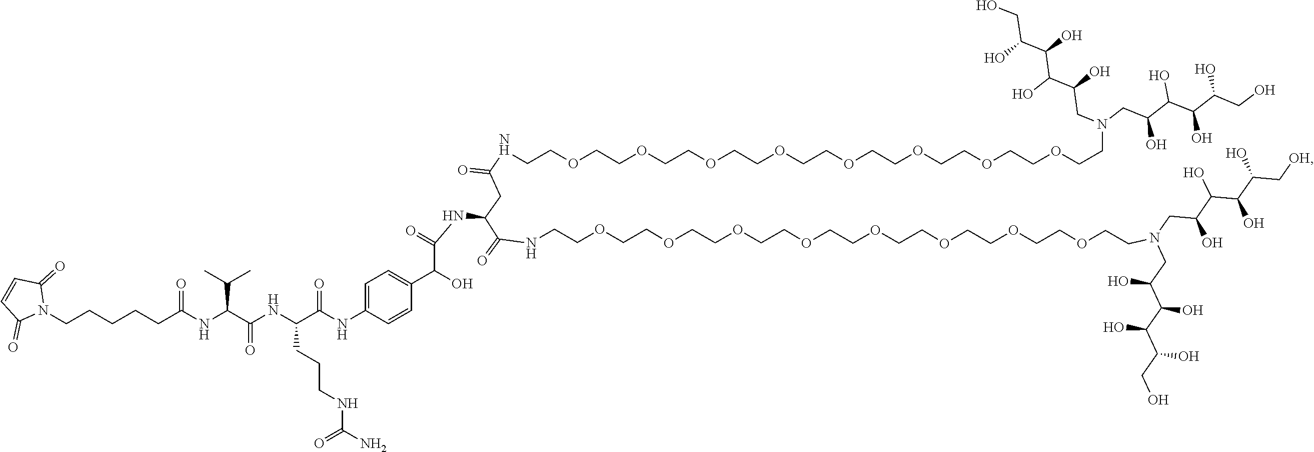 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCOCCOCCN(C[C@H](O)C(O)[C@H](O)[C@H](O)CO)C[C@H](O)C(O)[C@H](O)[C@H](O)CO)C(=O)NCCOCCOCCOCCOCCOCCOCCOCCOCCN(C[C@H](O)C(O)[C@H](O)[C@H](O)CO)C[C@H](O)[C@@H](O)[C@H](O)[C@H](O)CO)cc1]

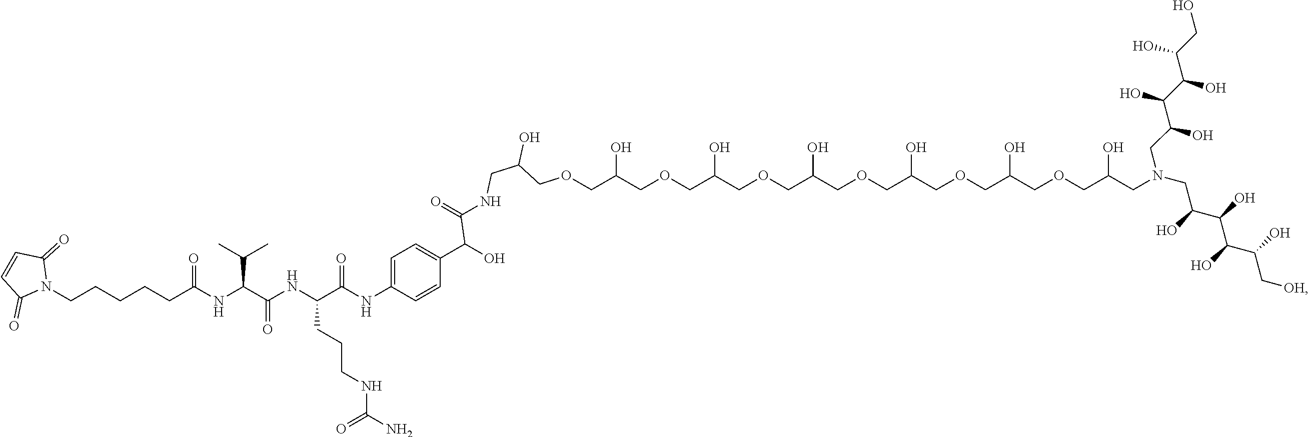 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

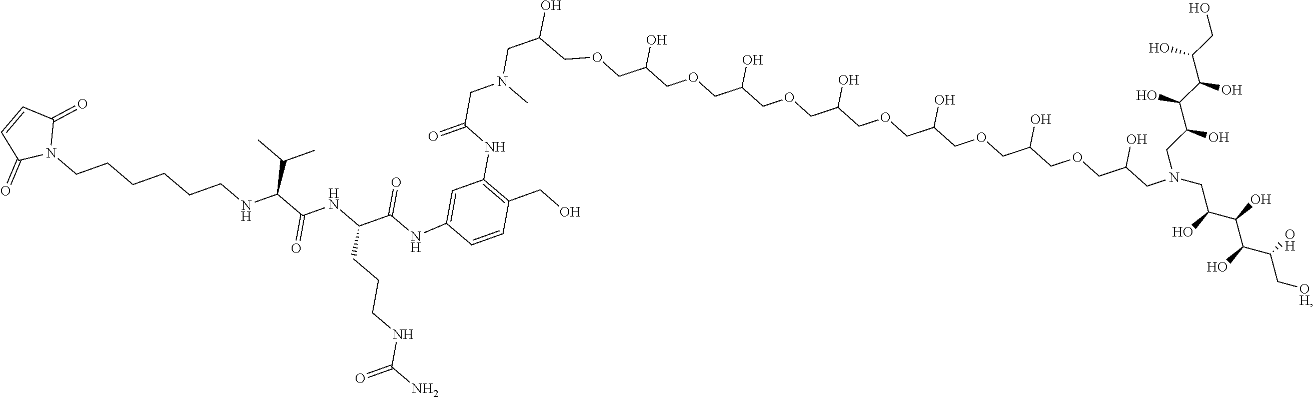 [SMILES: CC(C)[C@H](NCCCCCCN1C(=O)C=CC1=O)C(=O)N[C@@H](CCCNC(N)=O)C(=O)Nc1ccc(CO)c(NC(=O)CN(C)CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1]

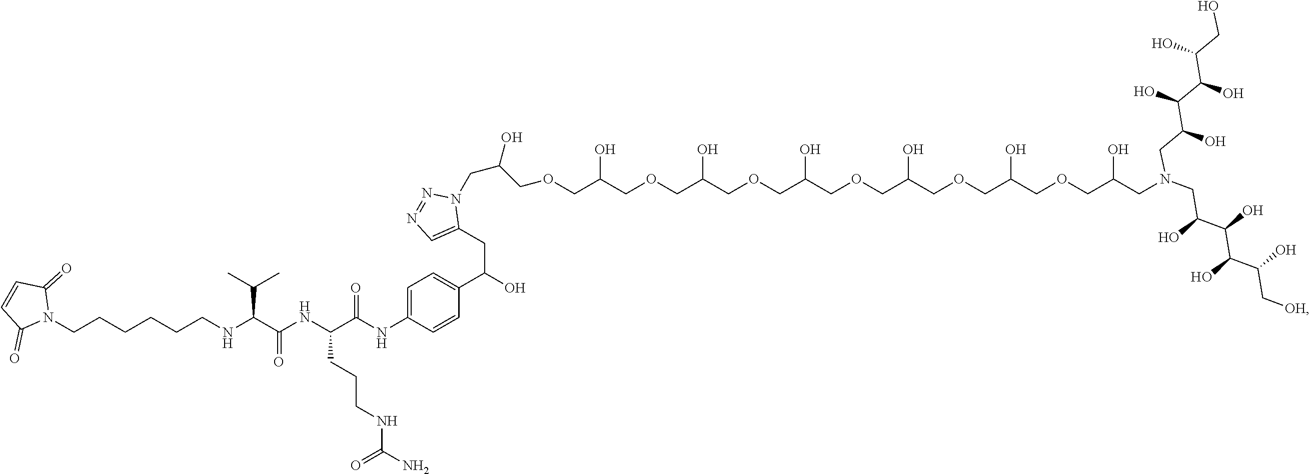 [SMILES: CC(C)[C@H](NCCCCCCN1C(=O)C=CC1=O)C(=O)N[C@@H](CCCNC(N)=O)C(=O)Nc1ccc(C(O)Cc2cnnn2CC(O)COCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

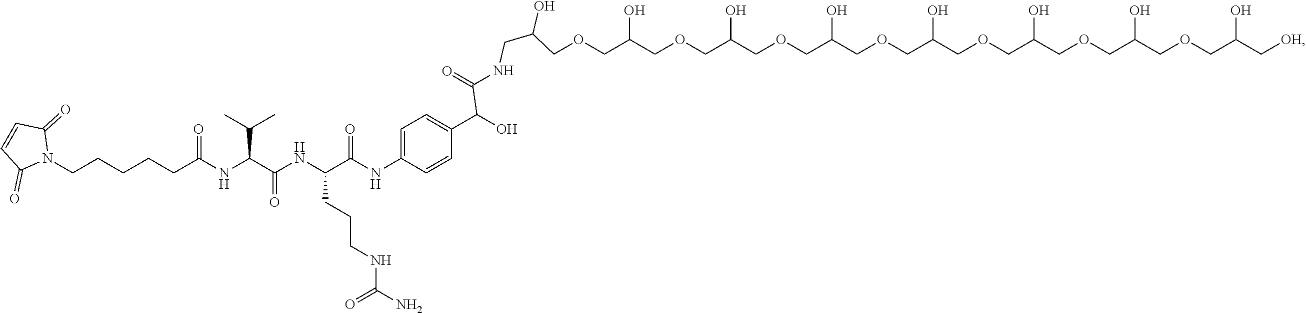 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCC(O)COCC(O)CO)cc1]

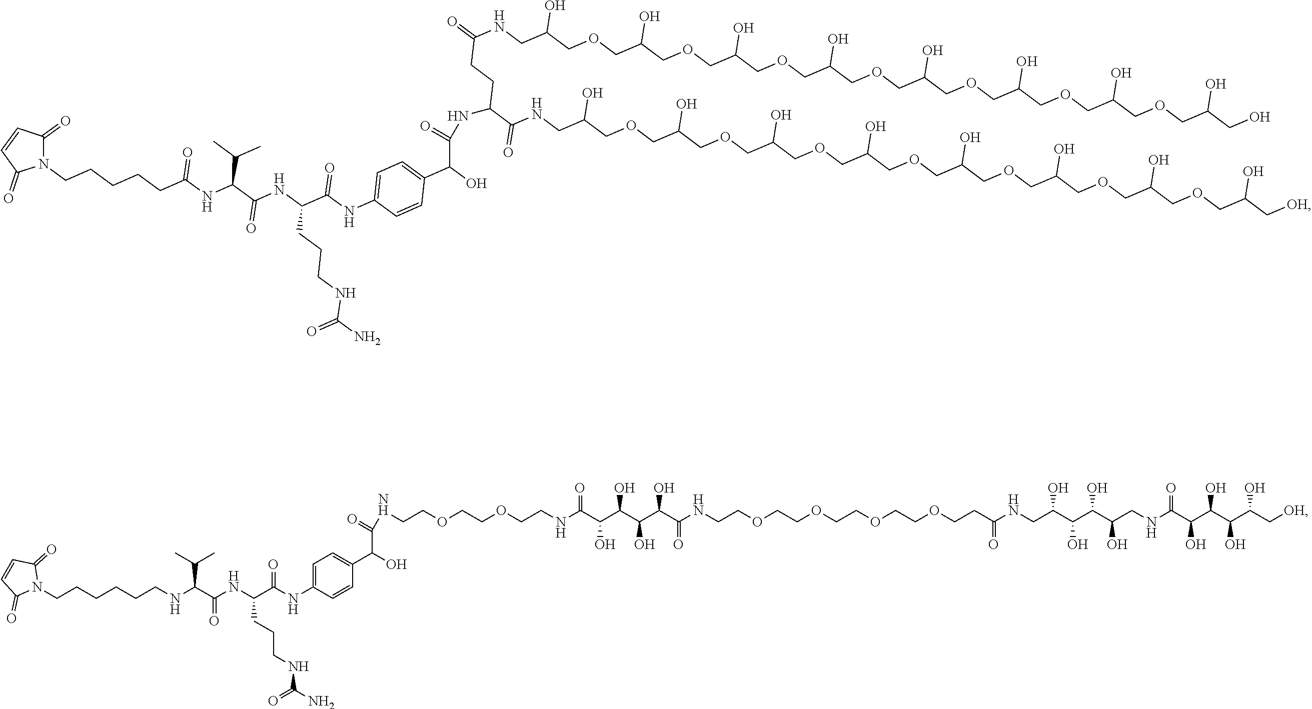

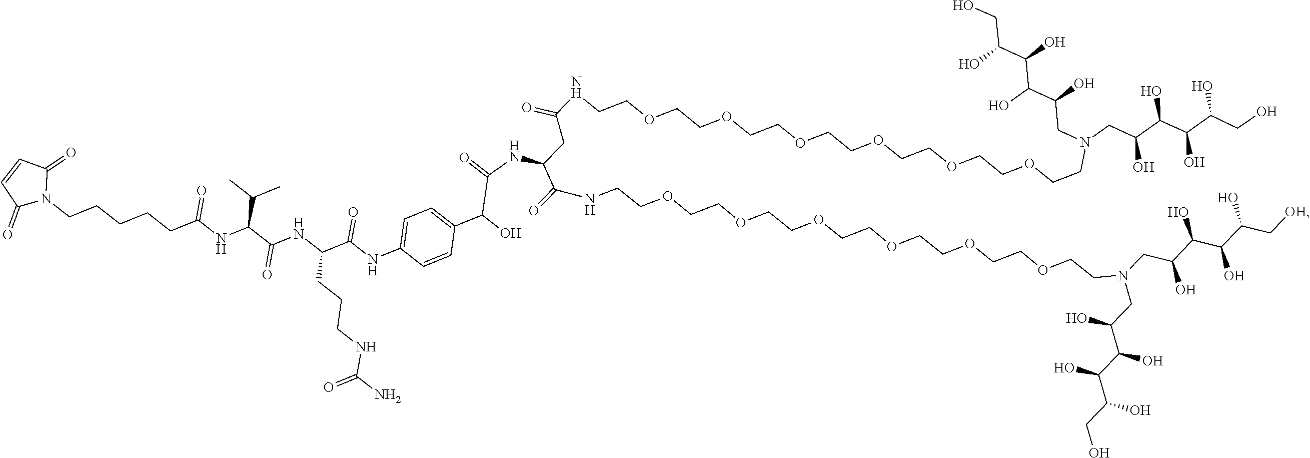 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCOCCOCCN(C[C@H](O)C(O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

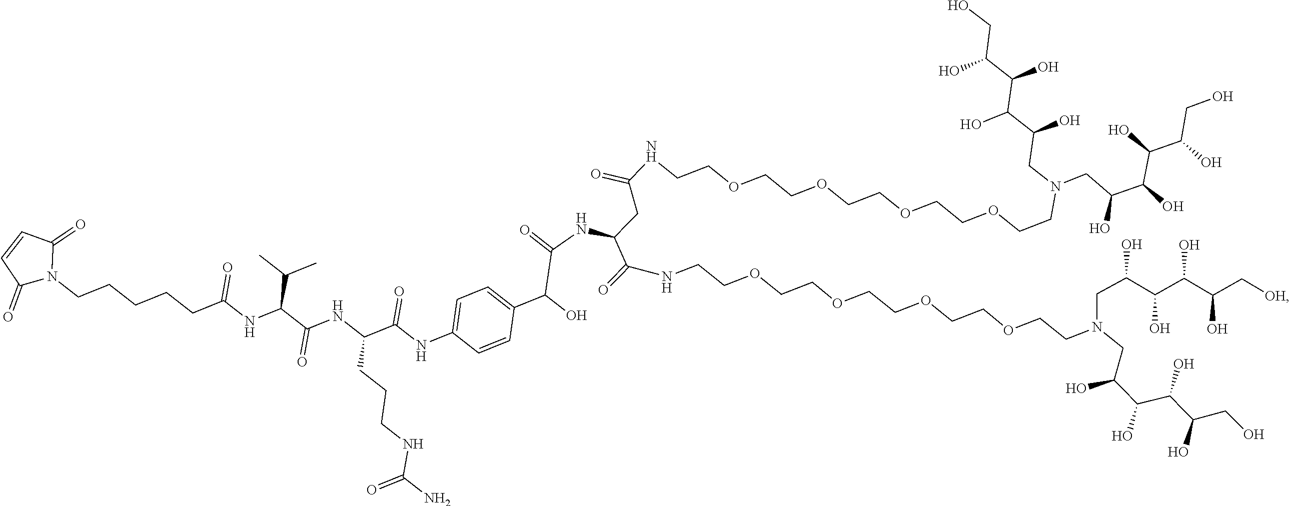 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@@H](CC(=O)NCCOCCOCCOCCOCCN(C[C@H](O)C(O)[C@H](O)[C@H](O)CO)C[C@H](O)[C@H](O)[C@@H](O)[C@@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

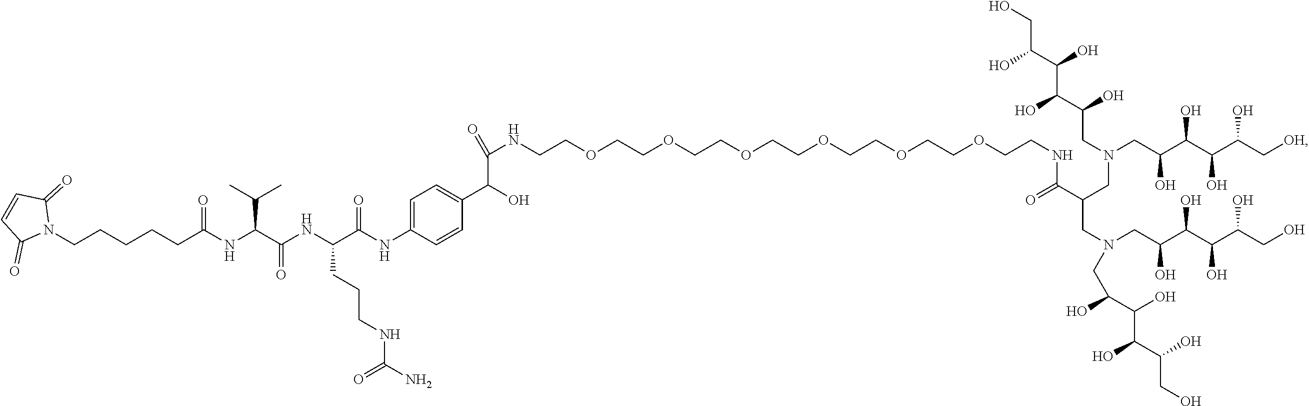 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCOCCOCCOCCOCCNC(=O)C(CN(C[C@H](O)C(O)[C@H](O)[C@H](O)CO)C[C@H](O)[C@@H](O)[C@H](O)[C@H](O)CO)CN(C[C@H](O)[C@@H](O)[C@H](O)[C@H](O)CO)C[C@H](O)[C@@H](O)[C@H](O)[C@H](O)CO)cc1]

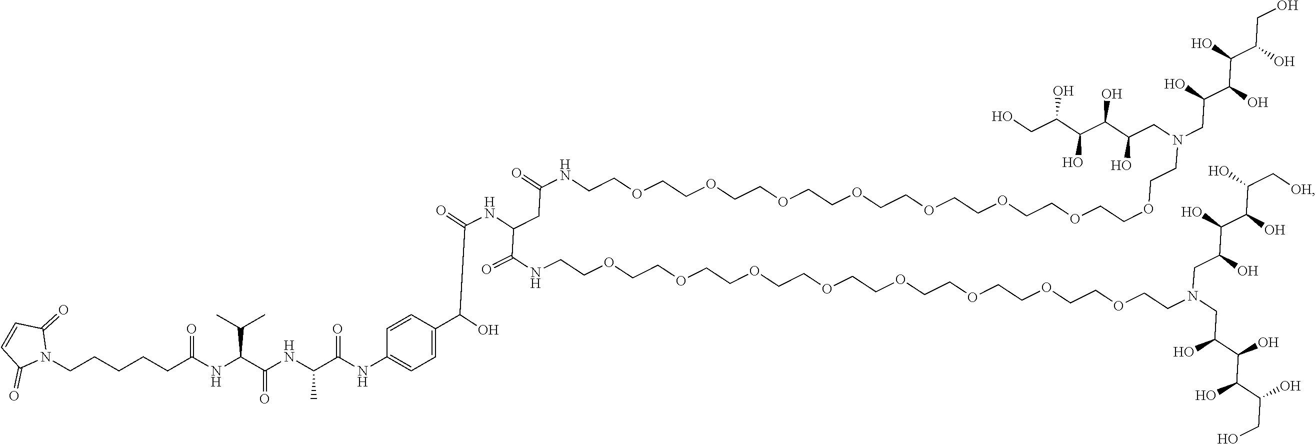 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

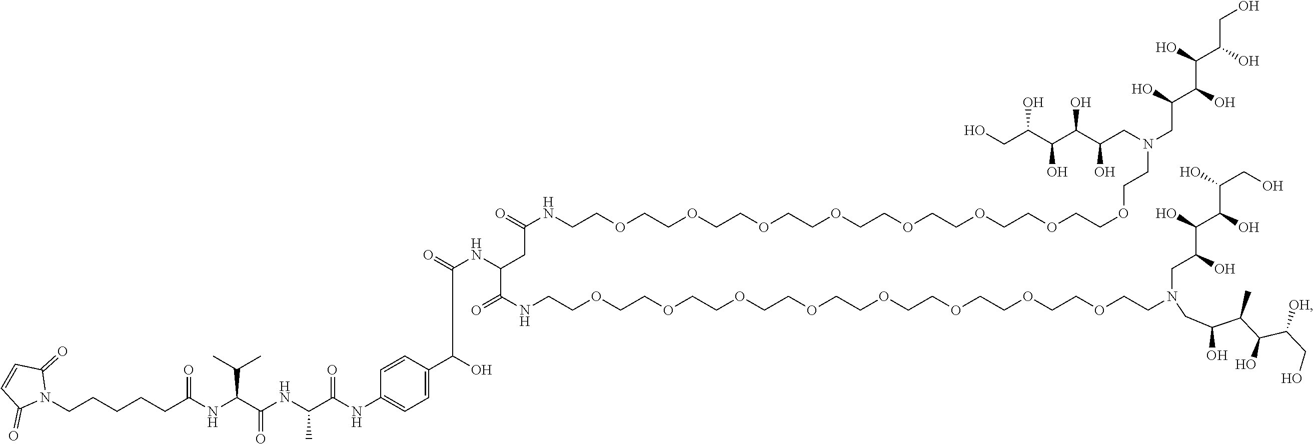 [SMILES: CC(C)[C@H](NC(=O)CCCCCN1C(=O)C=CC1=O)C(=O)N[C@@H](C)C(=O)Nc1ccc(C(O)C(=O)NC(CC(=O)NCCOCCOCCOCCOCCOCCOCCOCCOCCN(C[C@@H](O)[C@H](O)[C@@H](O)[C@@H](O)CO)C[C@@H](O)[C@H](O)[C@@H](O)[C@@H](O)CO)C(=O)NCCOCCOCCOCCOCCOCCOCCOCCOCCN(C[C@H](O)[C@@H](C)[C@H](O)[C@H](O)CO)C[C@H](O)[C@@H](O)[C@H](O)[C@H](O)CO)cc1]

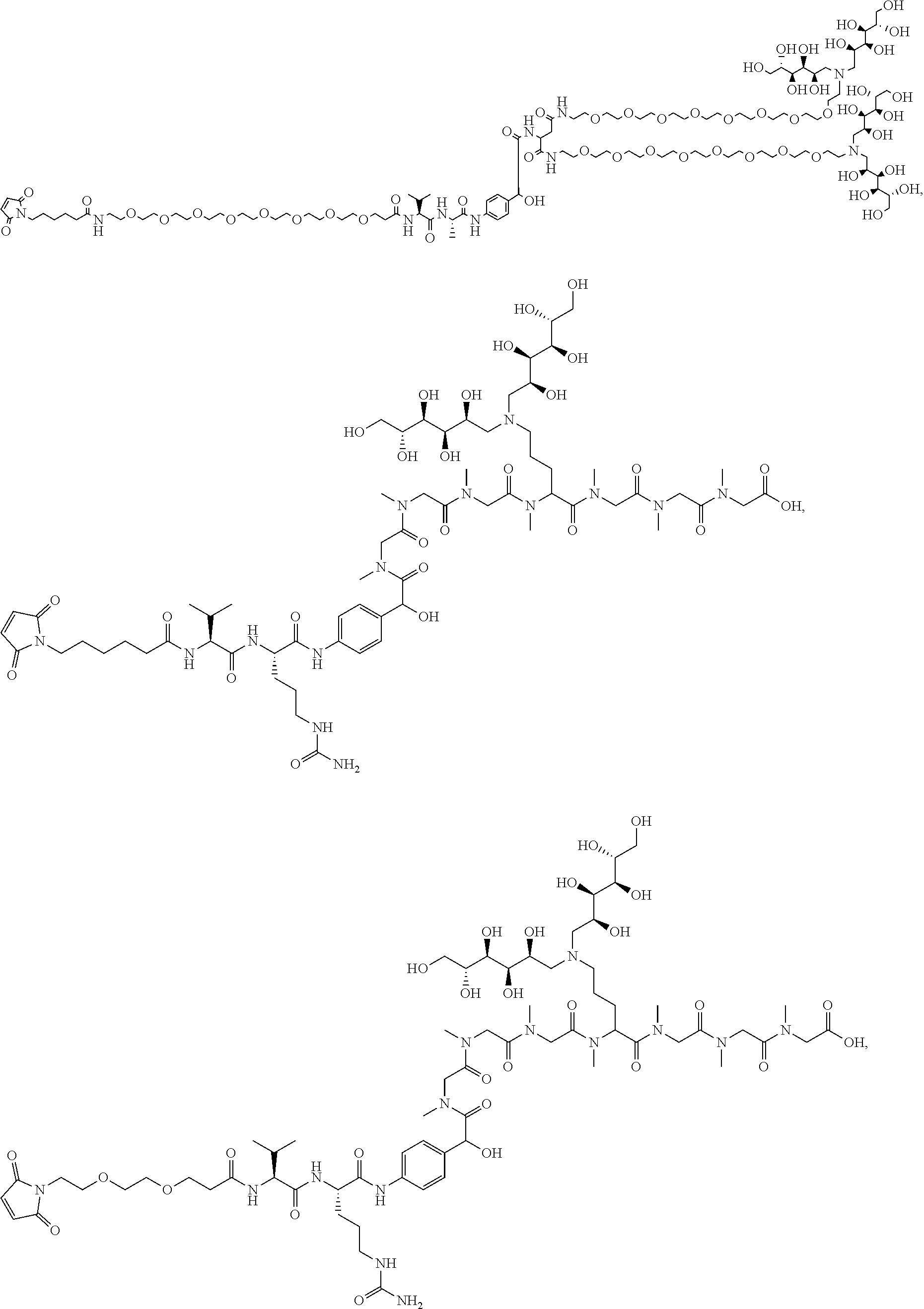

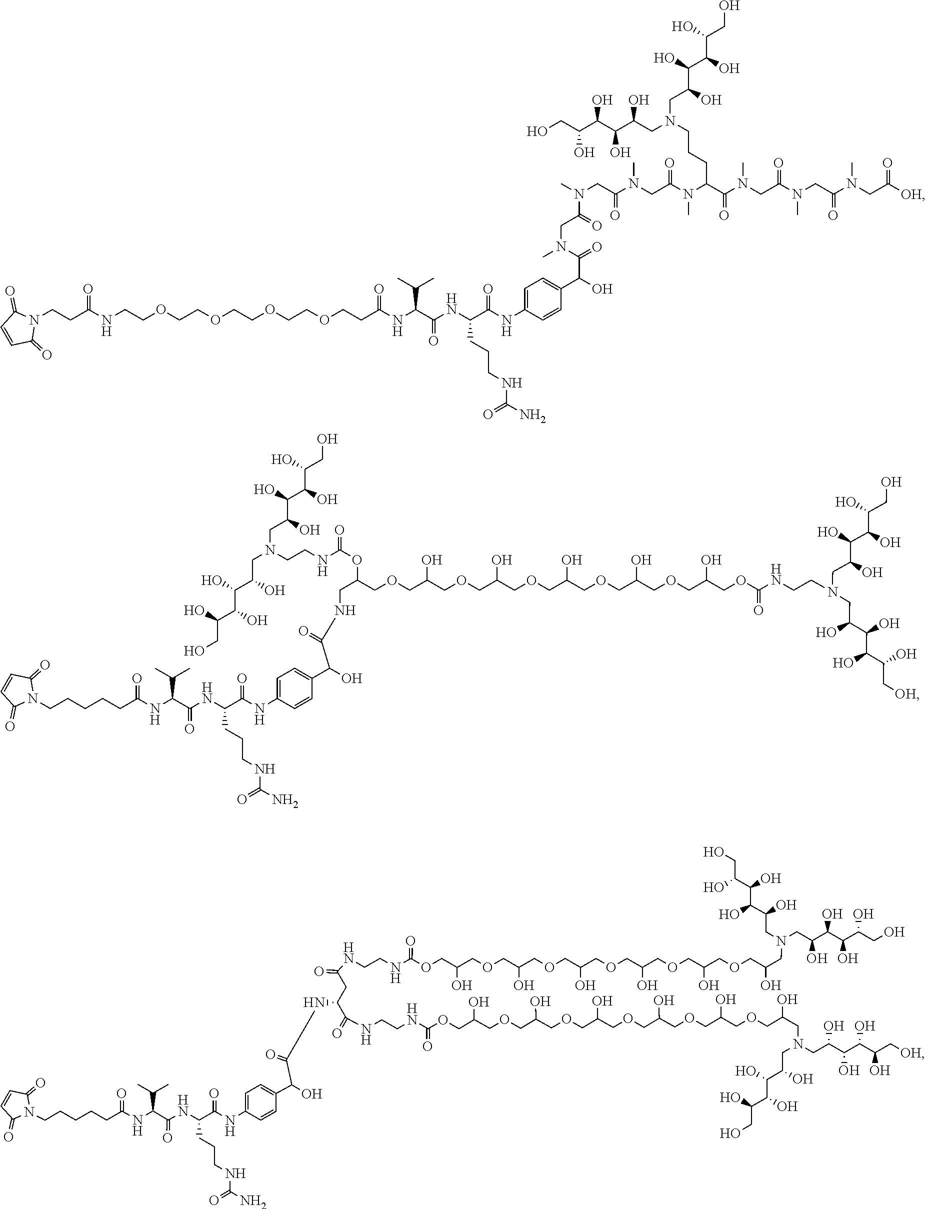

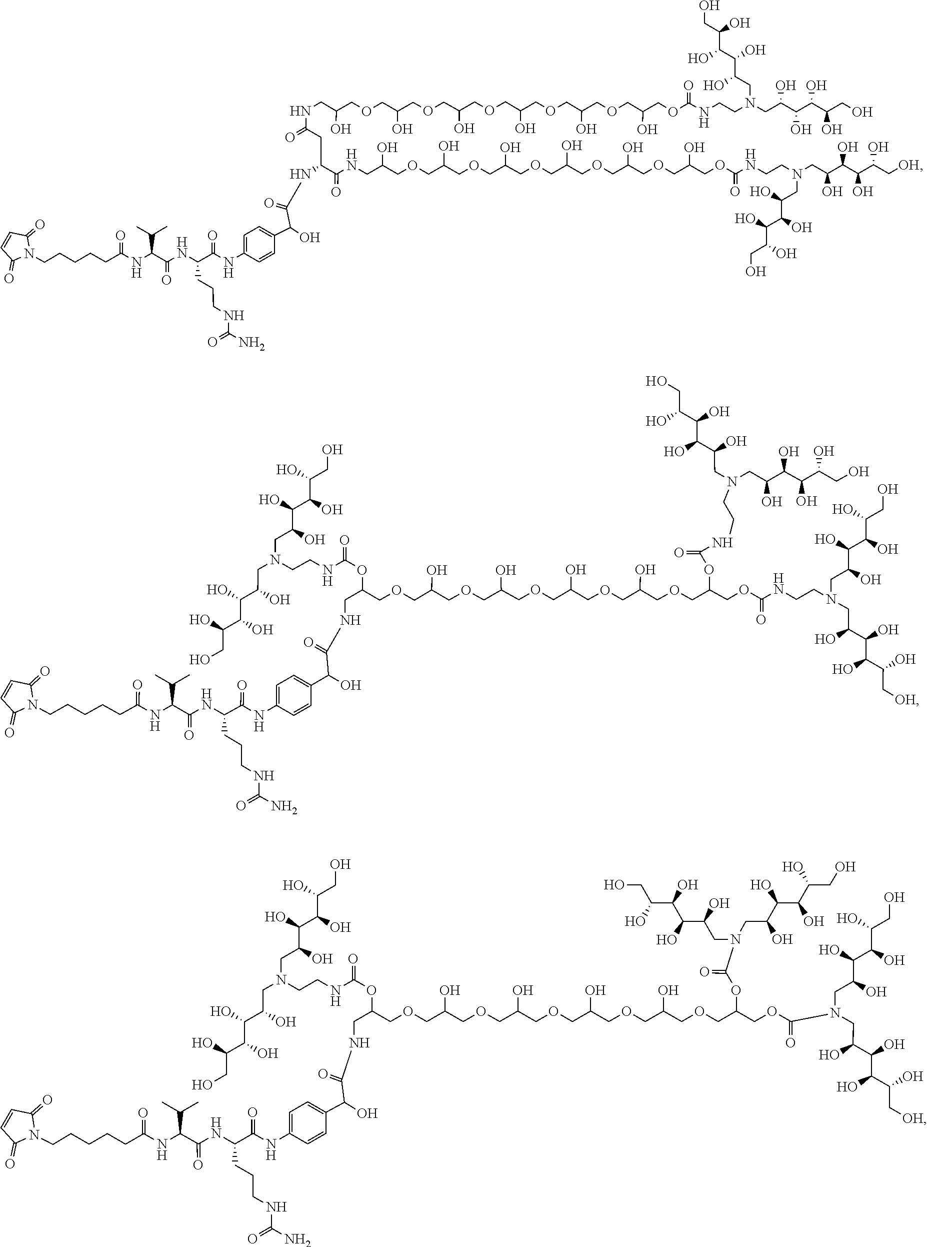

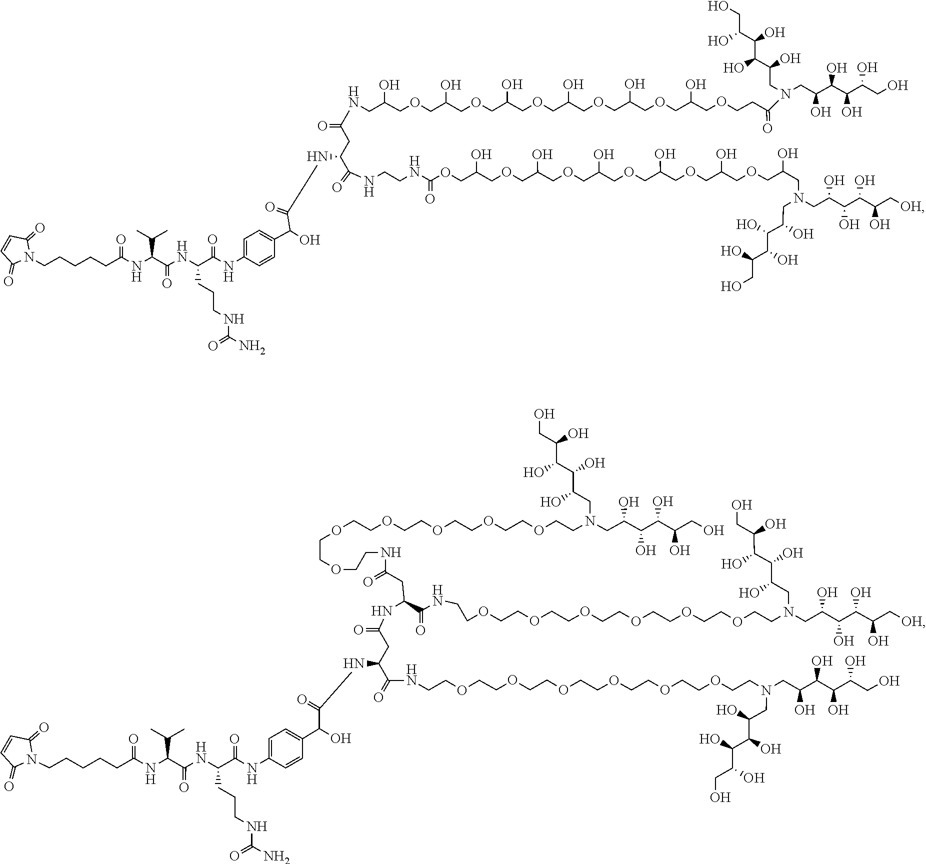

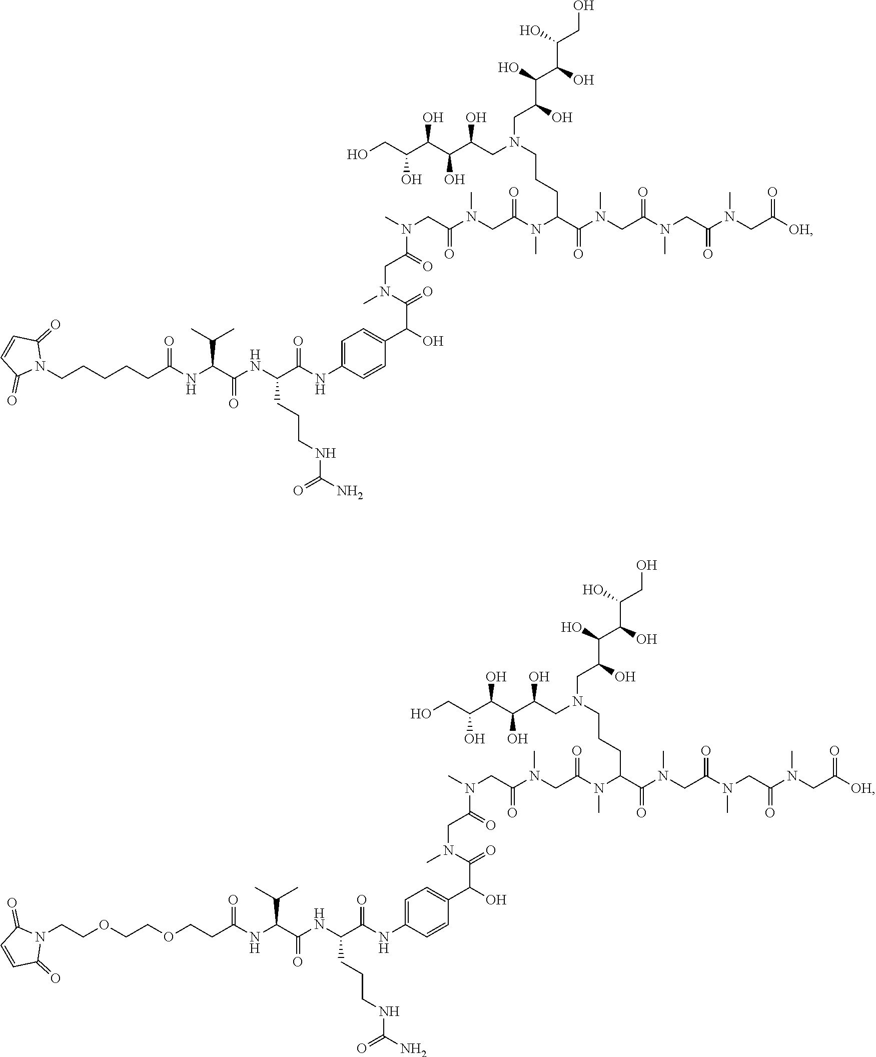 [SMILES: CC(C)[C@H](NC(=O)CCCCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1 ; CC(C)[C@H](NC(=O)CCOCCOCCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1]

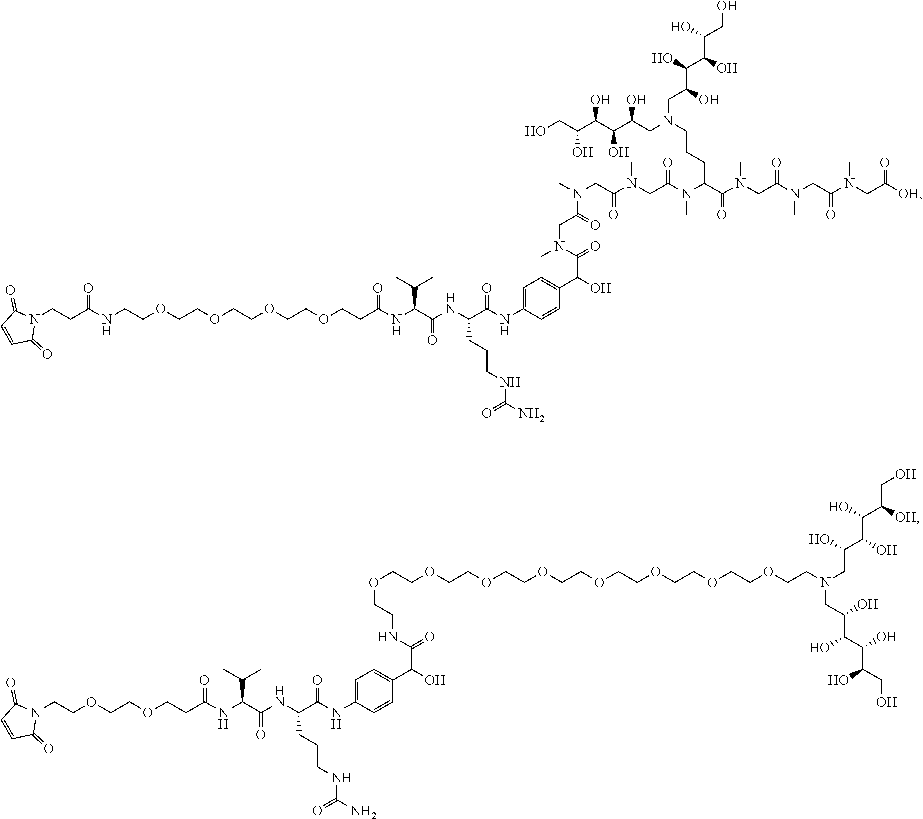 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)O)cc1 ; CC(C)[C@H](NC(=O)CCOCCOCCN1C(=O)C=CC1=O)C(=O)N[C@@H](CCCNC(N)=O)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

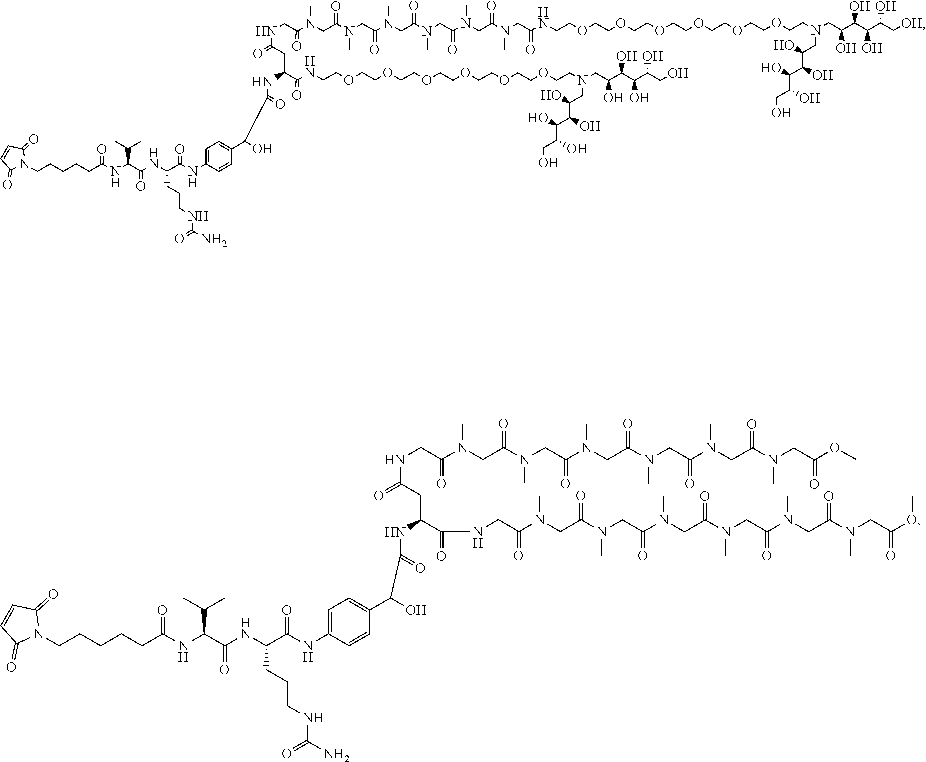

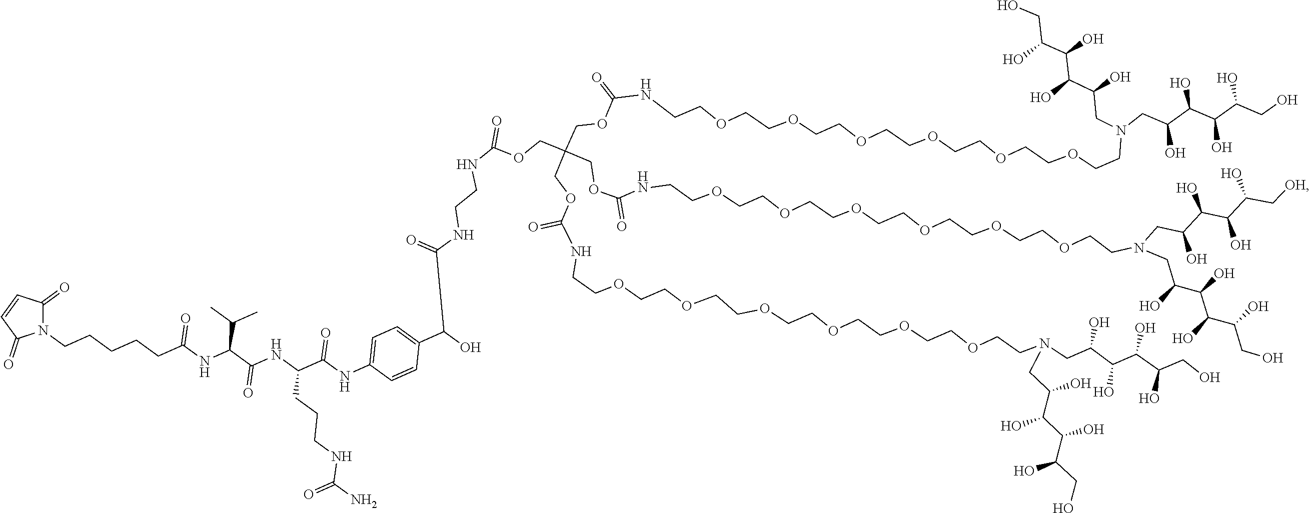 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCNC(=O)OCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

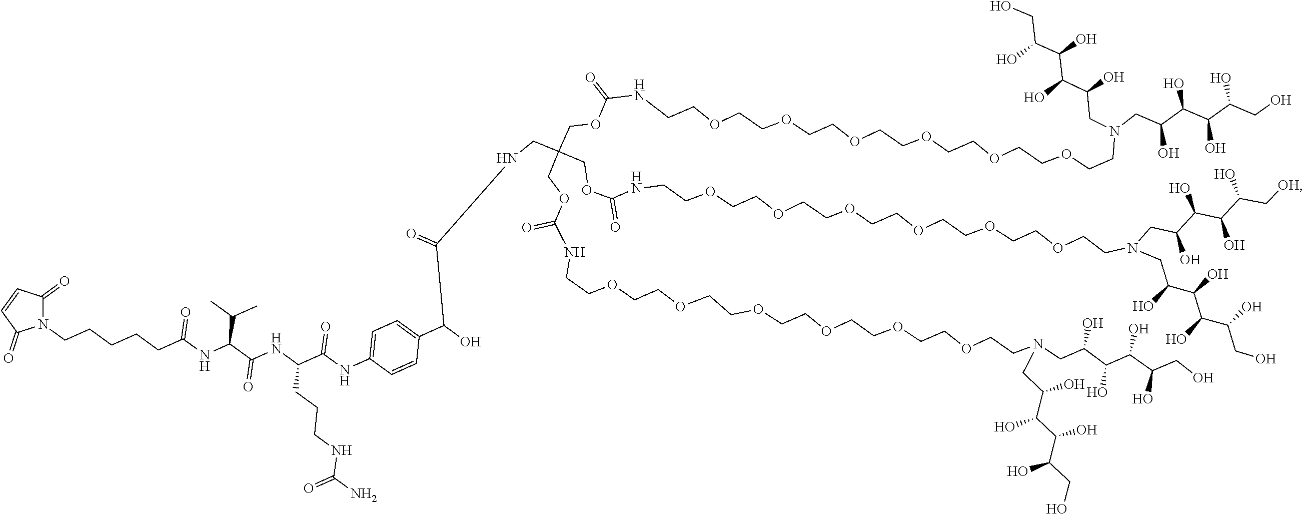 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)COC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

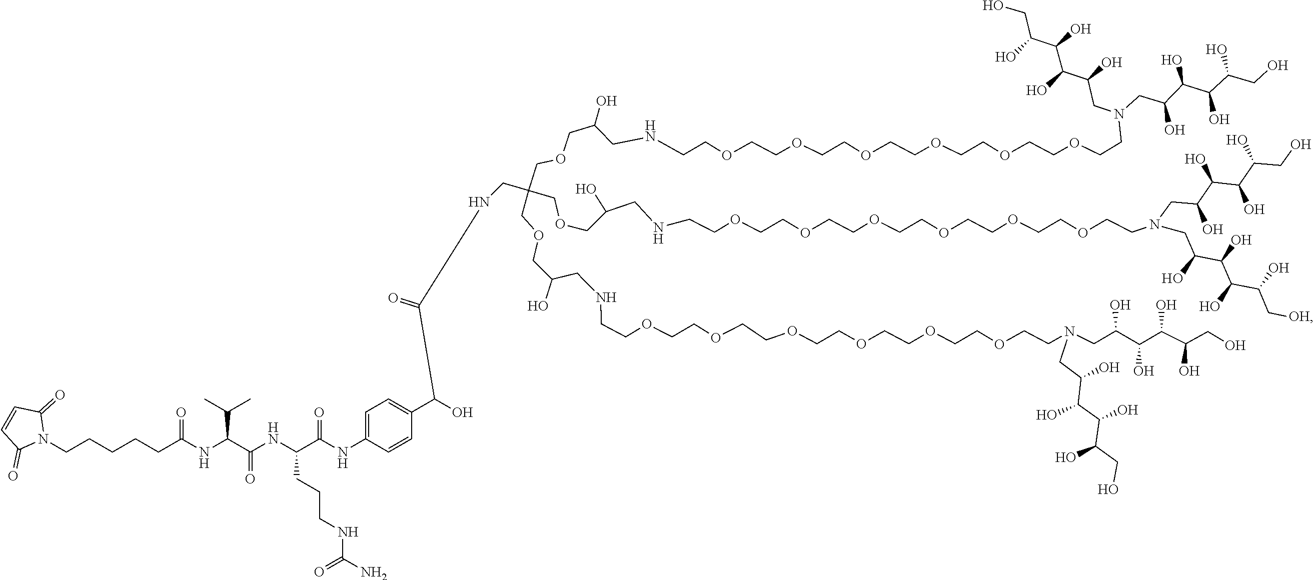 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)(COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)COCC(O)CNCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

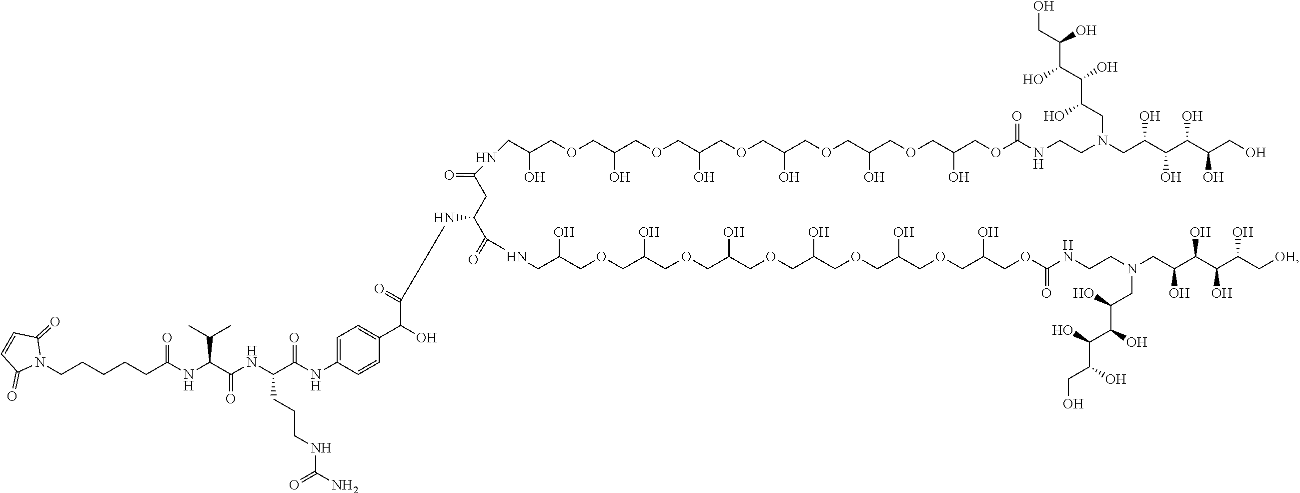 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

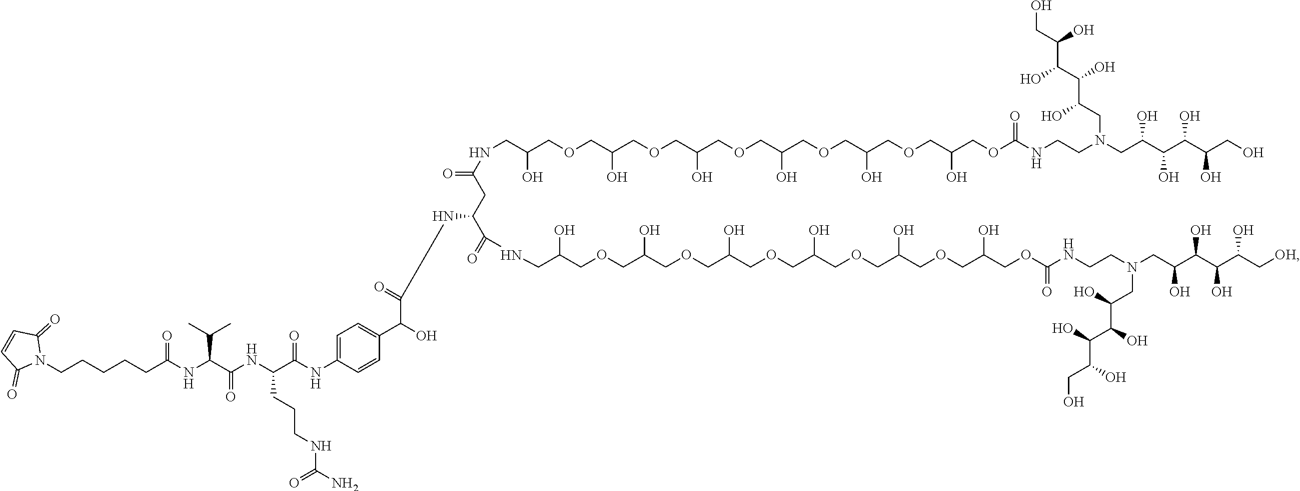 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)COC(=O)NCCN(C[C@H](O)[C@@H](O)[C@H](O)[C@H](O)CO)C[C@H](O)[C@@H](O)[C@H](O)[C@H](O)CO)cc1]

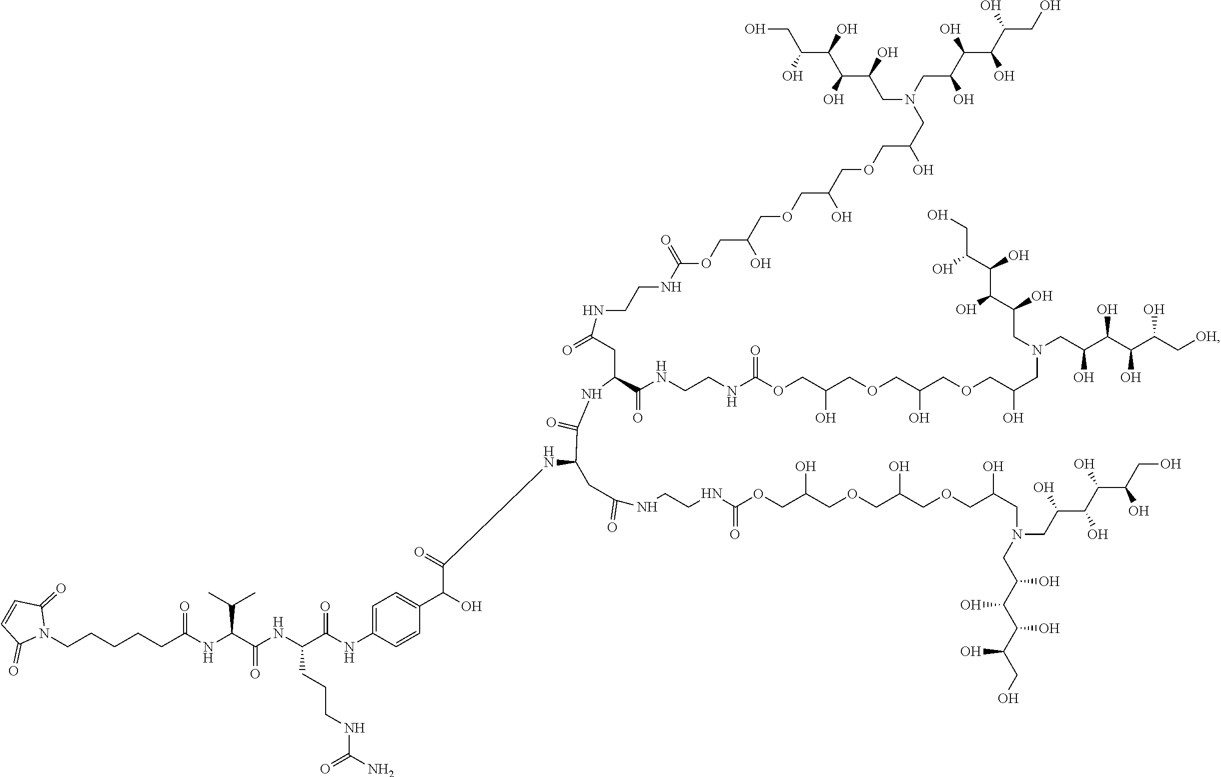 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

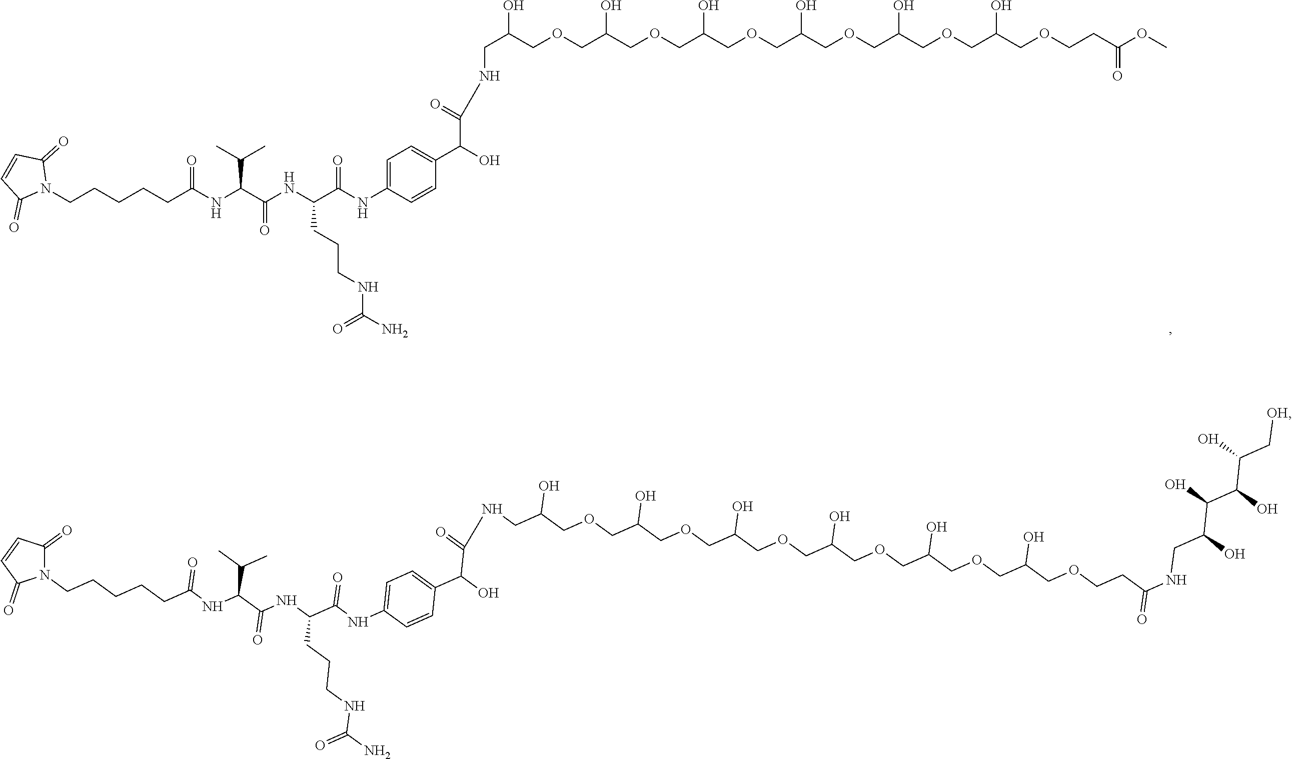 [SMILES: COC(=O)CCOCC(O)COCC(O)COCC(O)COCC(O)COCC(O)COCC(O)CNC(=O)C(O)c1ccc(NC(=O)[C@H](CCCNC(N)=O)NC(=O)[C@@H](NC(=O)CCCCCN2C(=O)C=CC2=O)C(C)C)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)COCCC(=O)NC[C@H](O)[C@@H](O)[C@H](O)[C@H](O)CO)cc1]

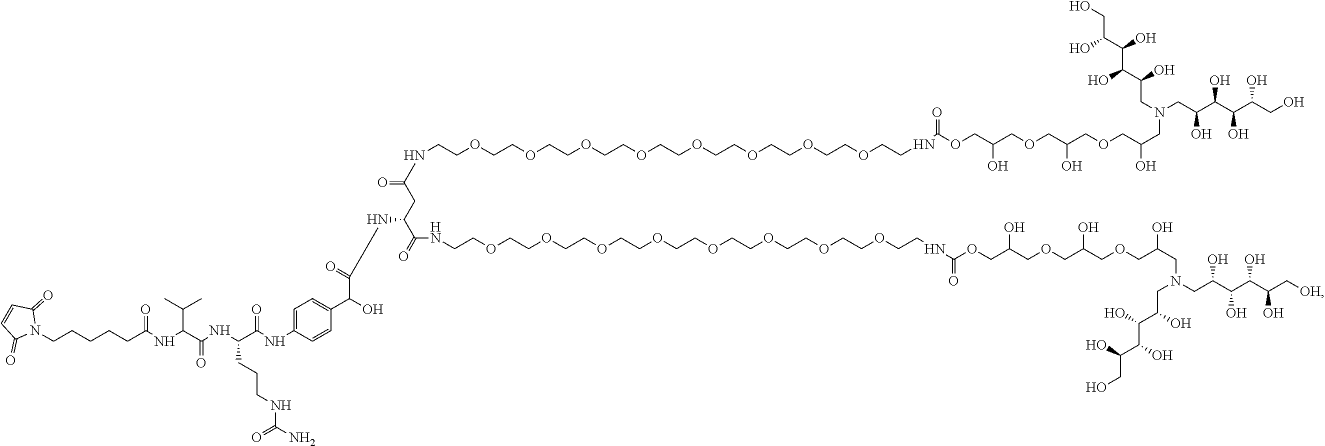 [SMILES: CC(C)C(NC(=O)CCCCCN1C(=O)C=CC1=O)C(=O)N[C@@H](CCCNC(N)=O)C(=O)Nc1ccc(C(O)C(=O)N[C@H](CC(=O)NCCOCCOCCOCCOCCOCCOCCOCCOCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

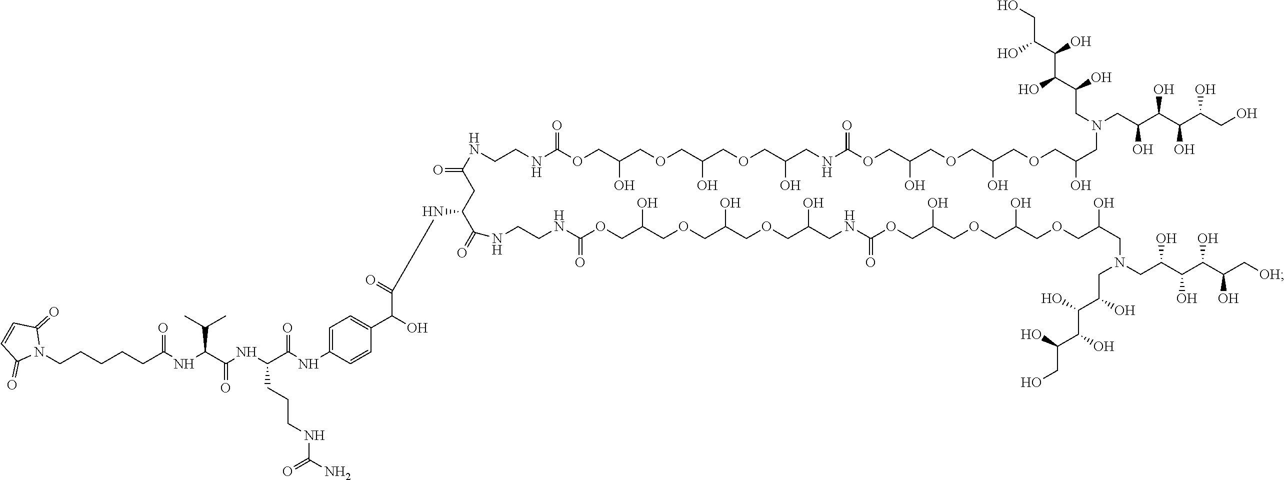 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

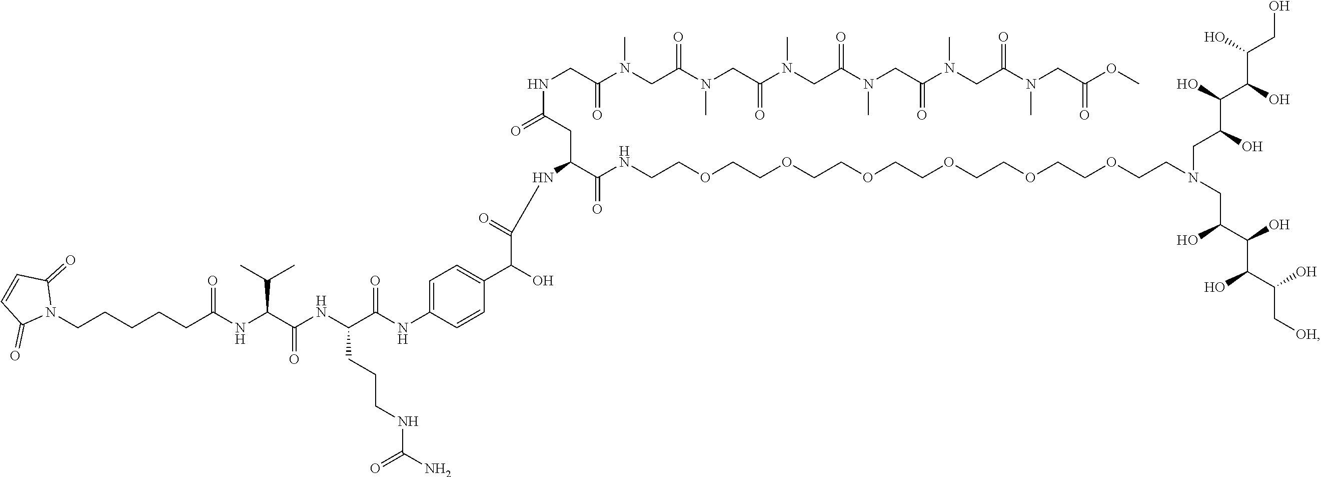 [SMILES: COC(=O)CN(C)C(=O)CN(C)C(=O)CN(C)C(=O)CN(C)C(=O)CN(C)C(=O)CN(C)C(=O)CNC(=O)C[C@H](NC(=O)C(O)c1ccc(NC(=O)[C@H](CCCNC(N)=O)NC(=O)[C@@H](NC(=O)CCCCCN2C(=O)C=CC2=O)C(C)C)cc1)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

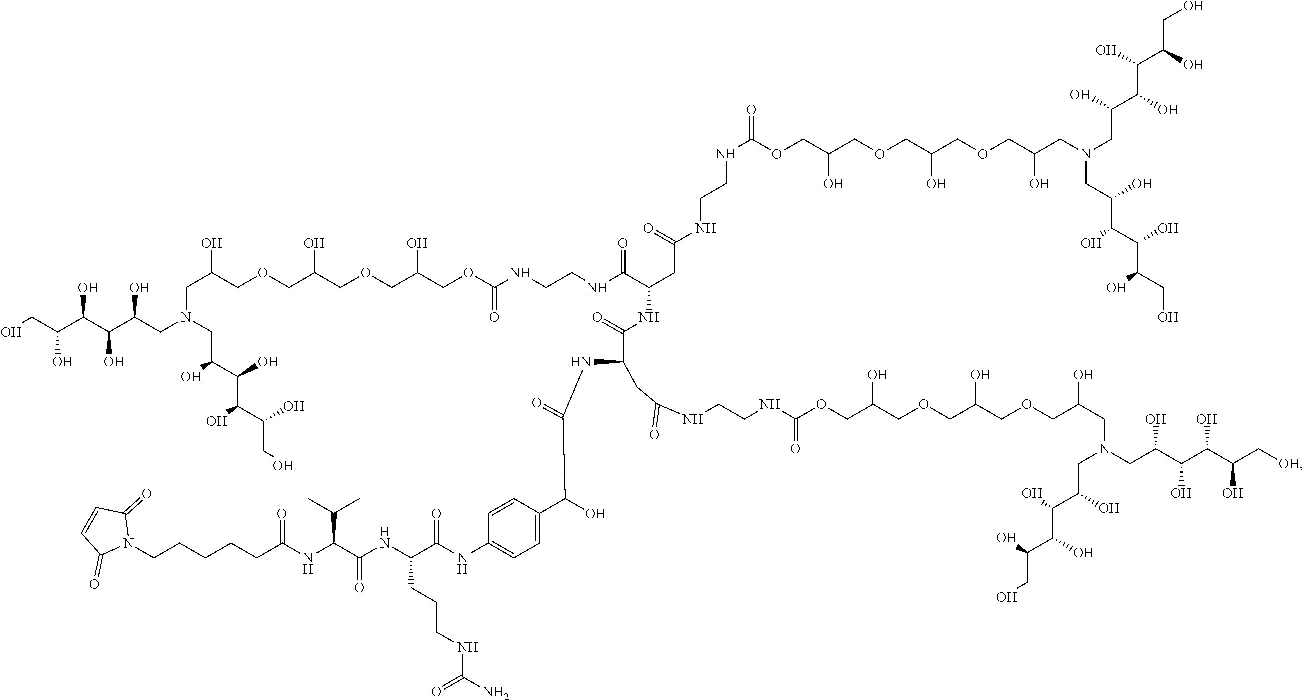 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

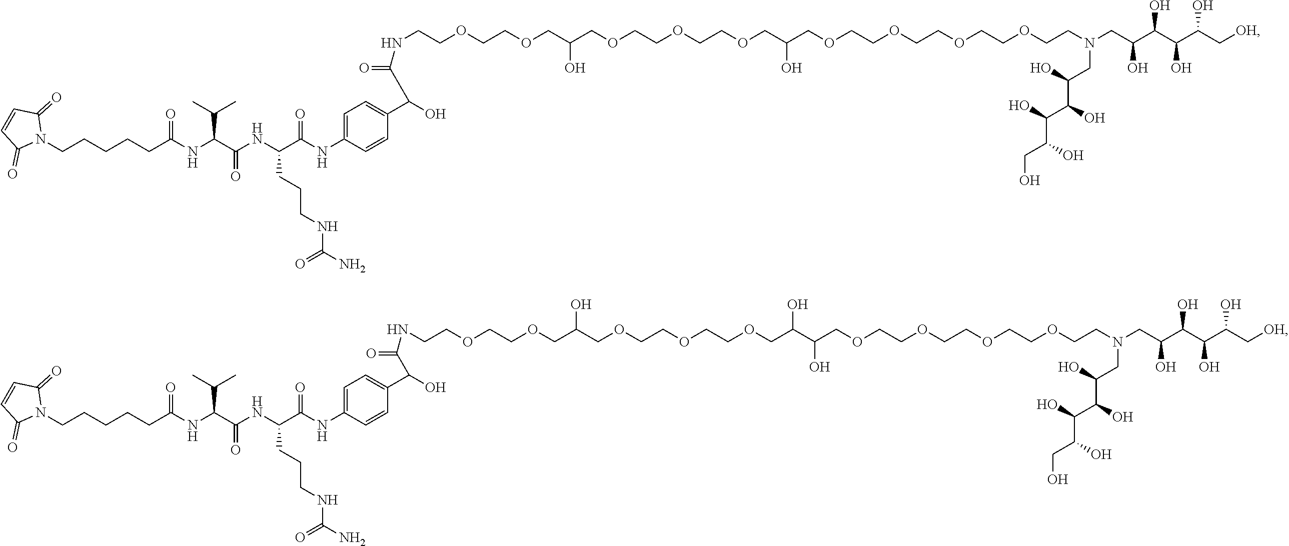 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)COCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCCCCN1C(=O)C=CC1=O)C(=O)N[C@@H](CCCNC(N)=O)C(=O)Nc1ccc(C(O)C(=O)NCCOCCOCC(O)COCCOCCOCC(O)C(O)COCCOCCOCCOCCN(C[C@H](O)[C@@H](O)[C@H](O)[C@H](O)CO)C[C@H](O)[C@@H](O)[C@H](O)[C@H](O)CO)cc1]

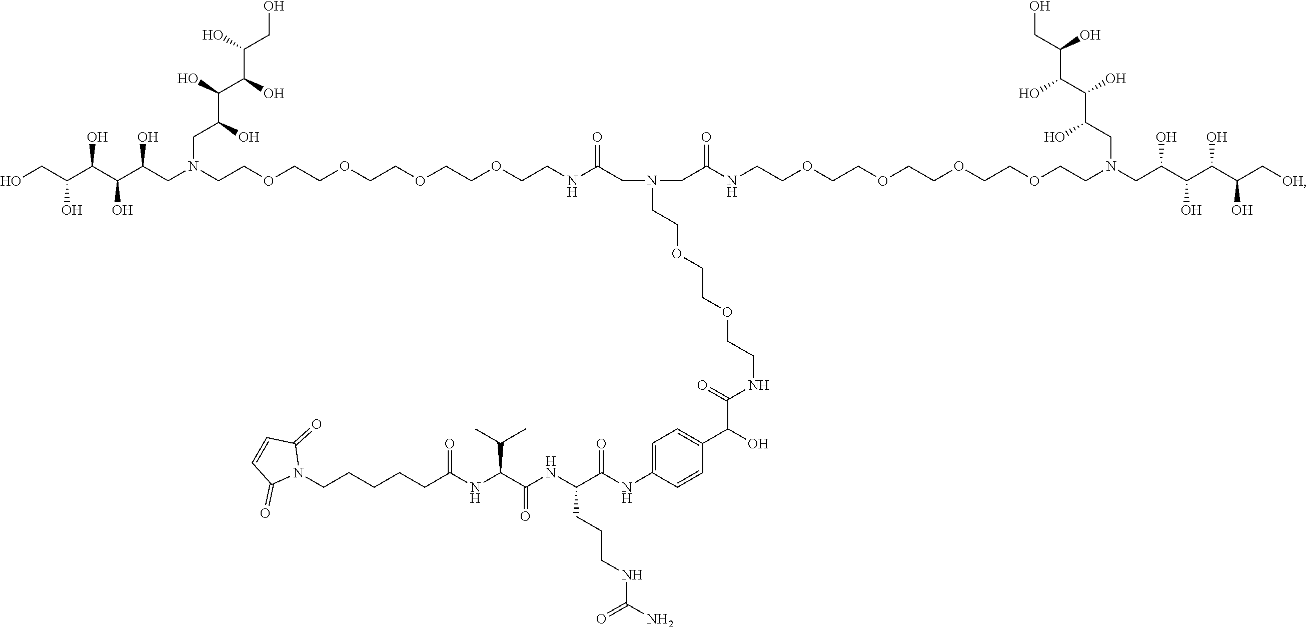 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCCOCCOCCN(CC(=O)NCCOCCOCCOCCOCCN(C[C@H](O)[C@@H](O)[C@H](O)[C@H](O)CO)C[C@H](O)[C@@H](O)[C@H](O)[C@H](O)CO)CC(=O)NCCOCCOCCOCCOCCN(C[C@H](O)[C@@H](O)[C@H](O)[C@H](O)CO)C[C@H](O)[C@@H](O)[C@H](O)[C@H](O)CO)cc1]

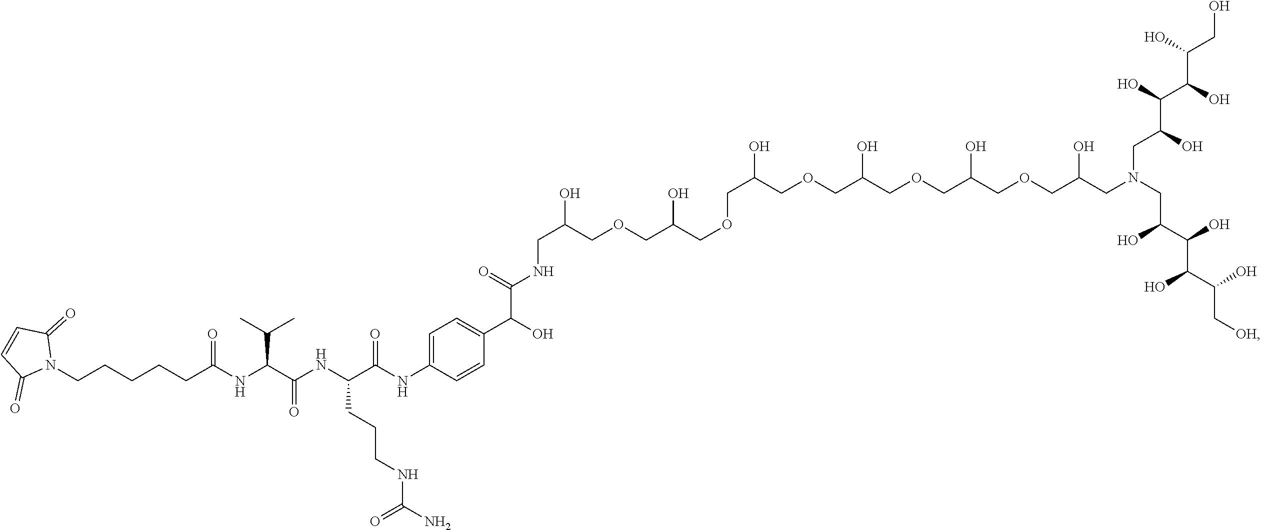 [SMILES: CC(C)[C@H](NC(=O)CCCCCN1C(=O)C=CC1=O)C(=O)N[C@@H](CCCNC(N)=O)C(=O)Nc1ccc(C(O)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

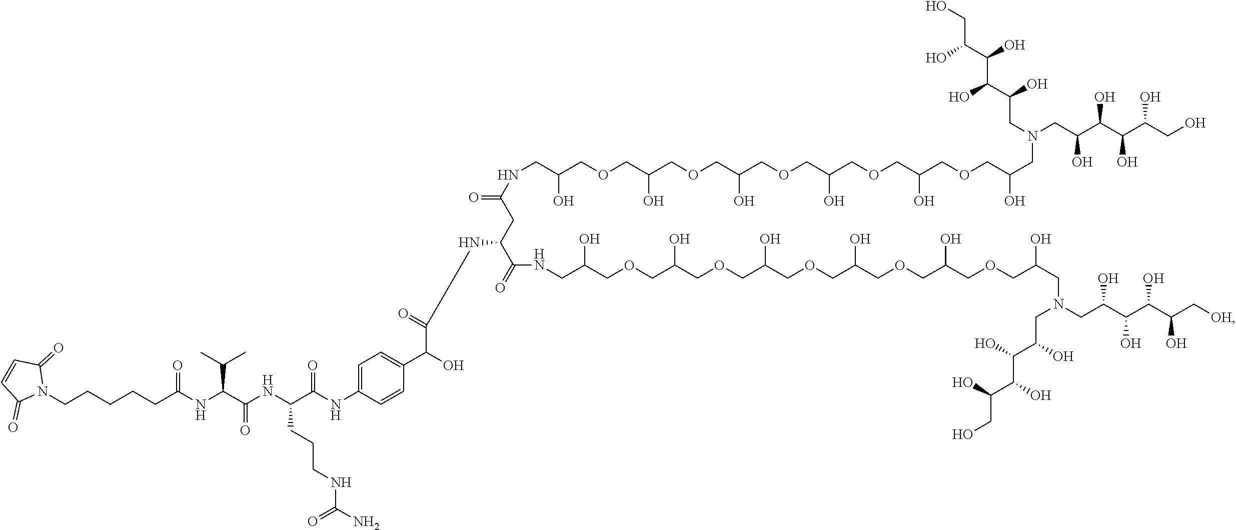 [SMILES: CC(C)[C@H](NC(=O)CCCCCN1C(=O)C=CC1=O)C(=O)N[C@@H](CCCNC(N)=O)C(=O)Nc1ccc(C(O)C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)cc1]

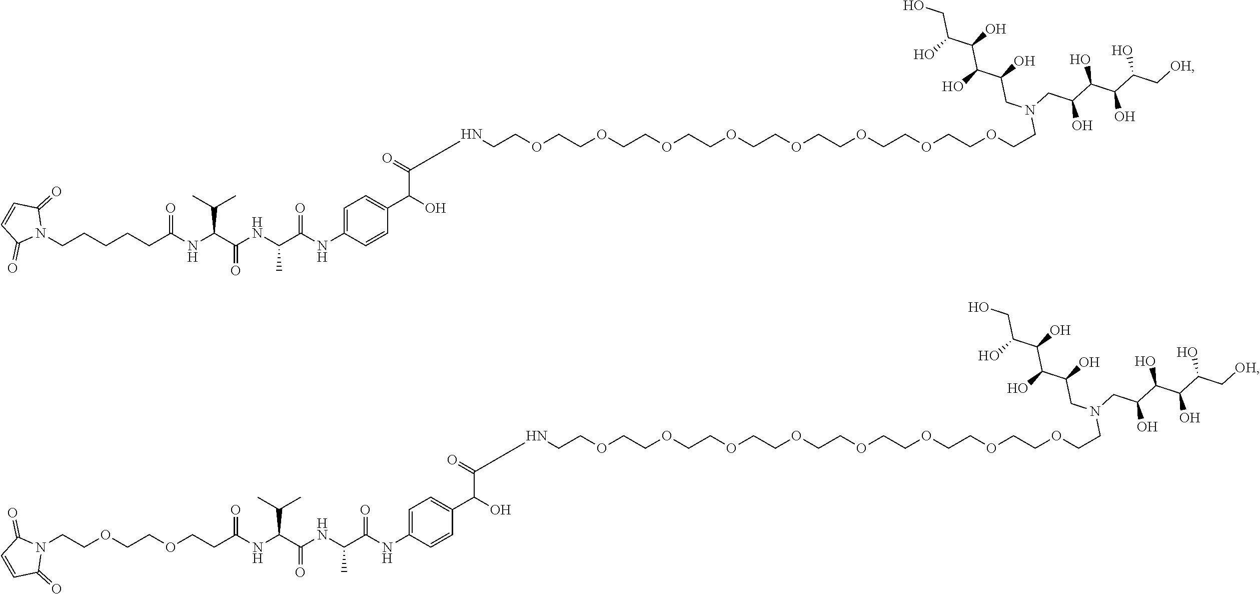 [SMILES: CC(C)[C@H](NC(=O)CCCCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1 ; CC(C)[C@H](NC(=O)CCOCCOCCN1C(=O)C=CC1=O)C(=O)N[C@@H](C)C(=O)Nc1ccc(C(O)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)cc1]

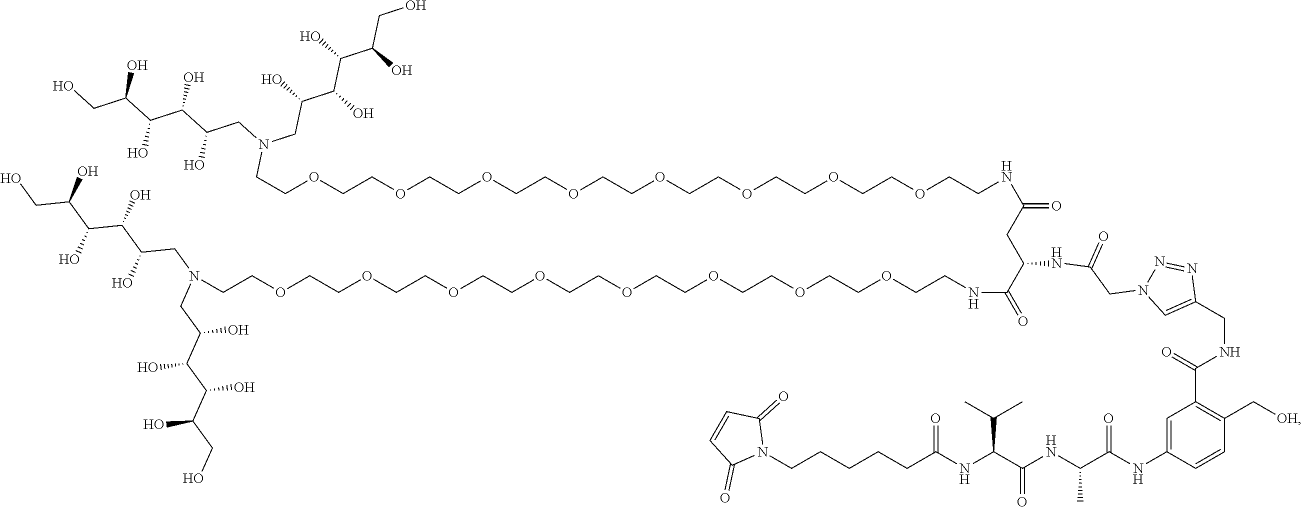

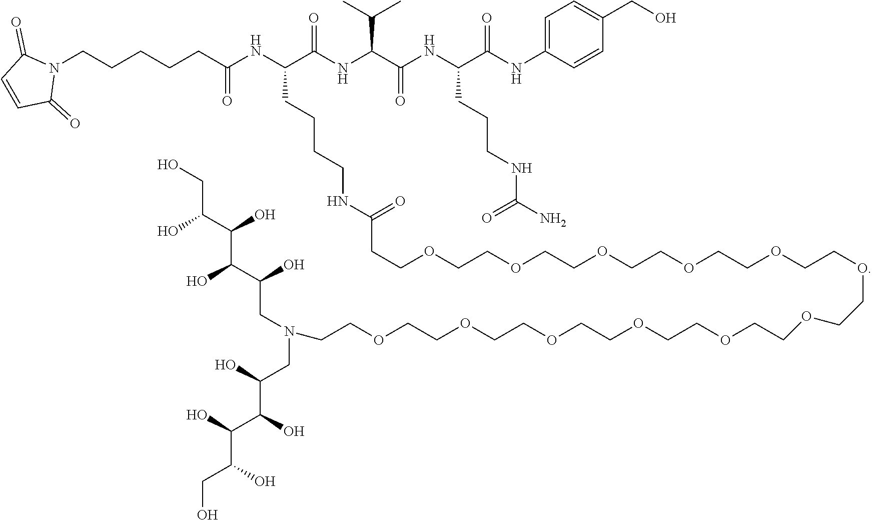 [SMILES: CC(C)[C@H](NC(=O)[C@H](CCCCNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CCCCCN1C(=O)C=CC1=O)C(=O)N[C@@H](CCCNC(N)=O)C(=O)Nc1ccc(CO)cc1]

wherein the H on the benzylic OH is optionally replaced with a bond to the at least one drug unit or to a linking group attached to the at least one drug unit.

Embodiment 108. The linker compound of any one of Embodiments 1-9, having one of the following structures:

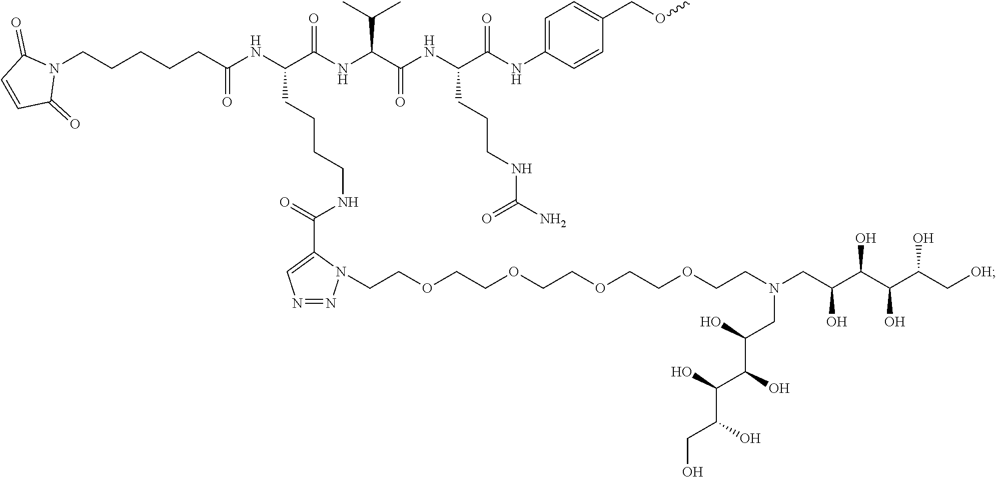

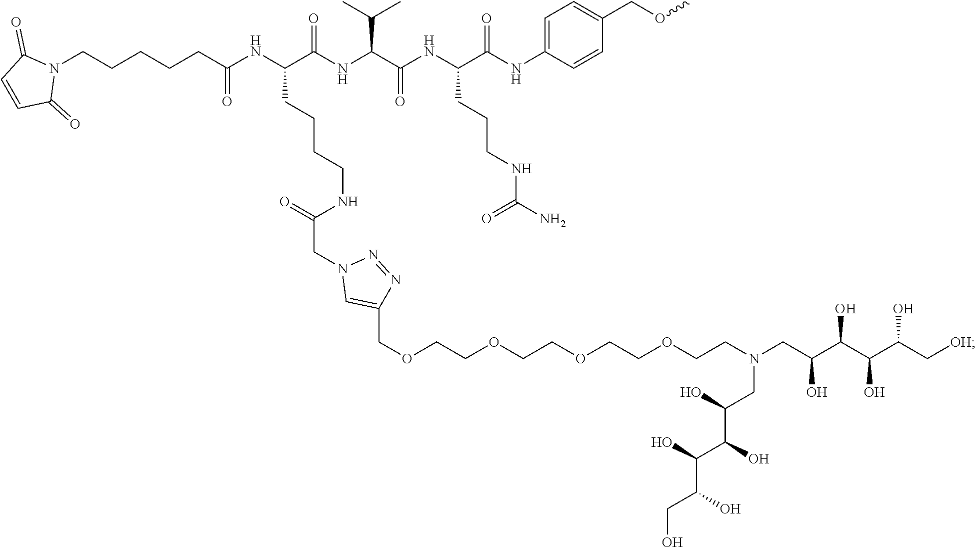

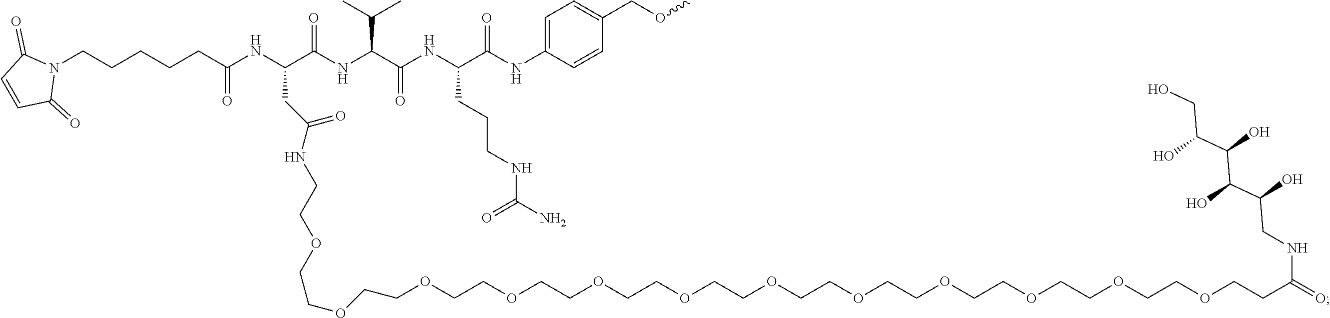

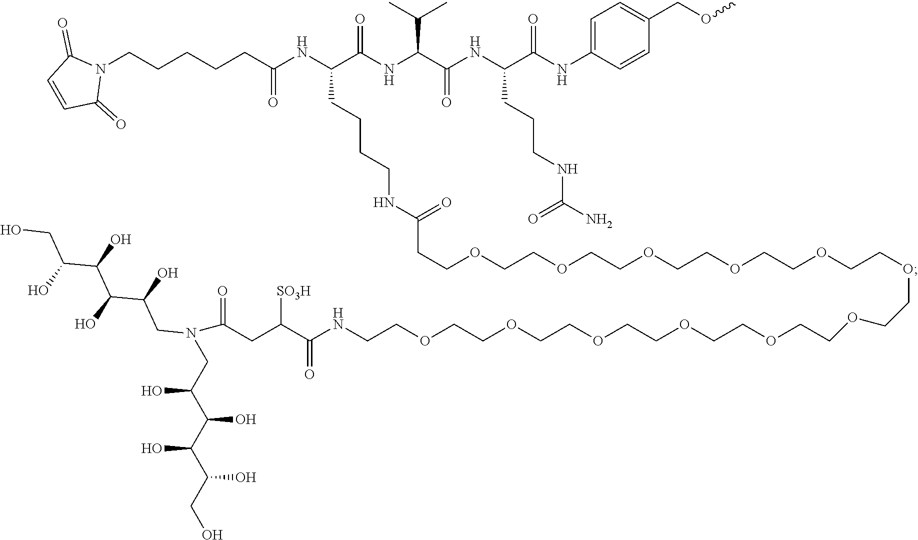

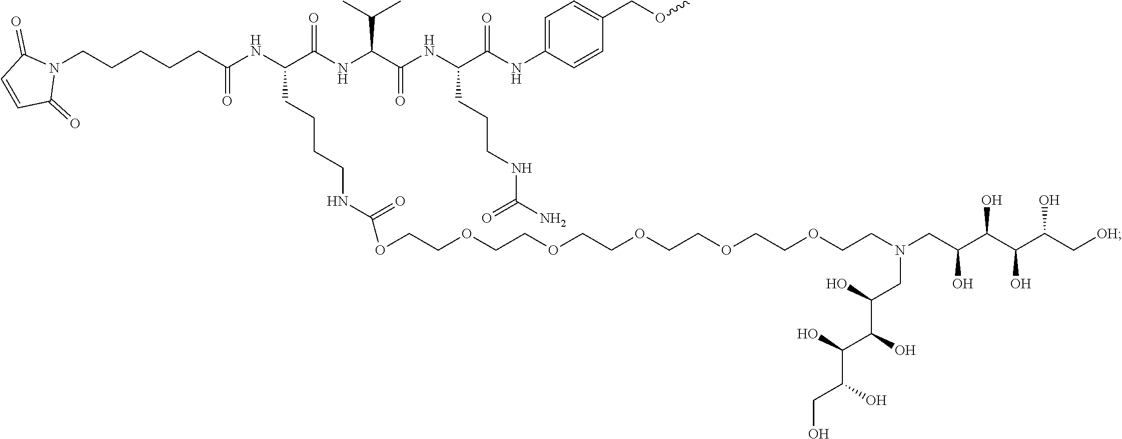

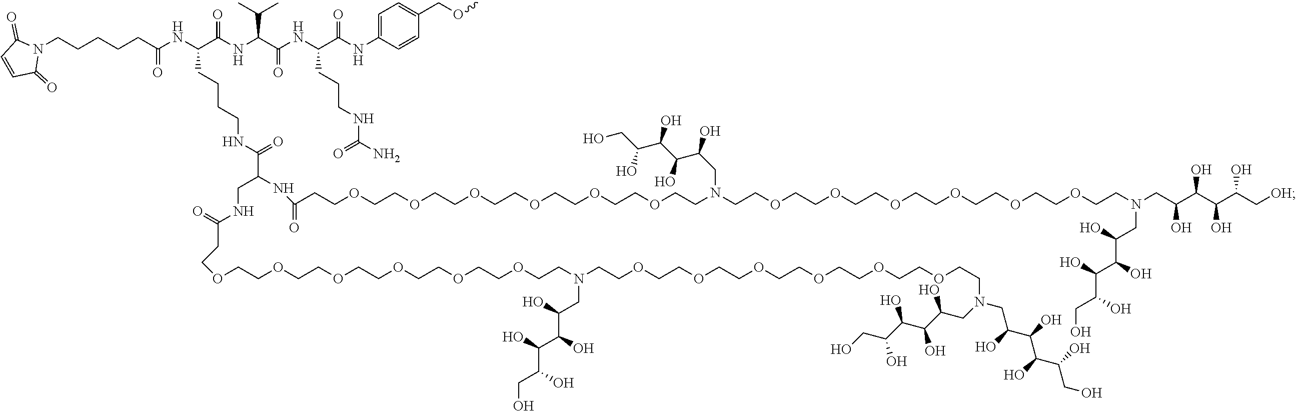

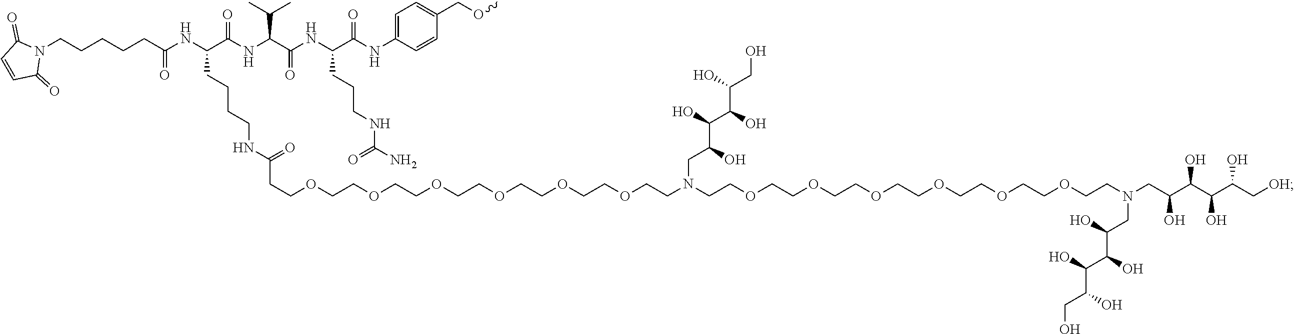

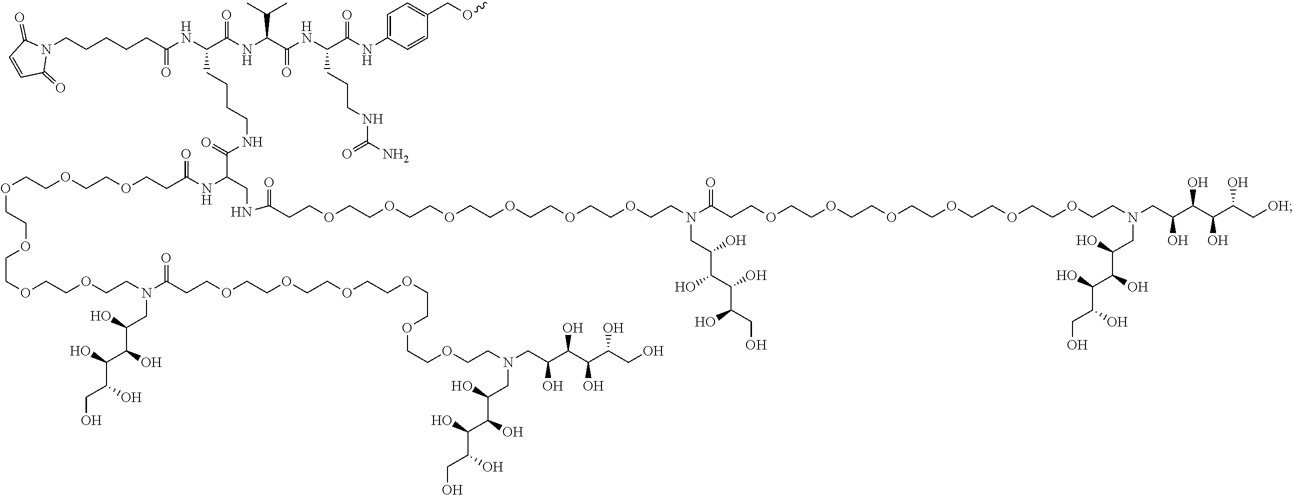

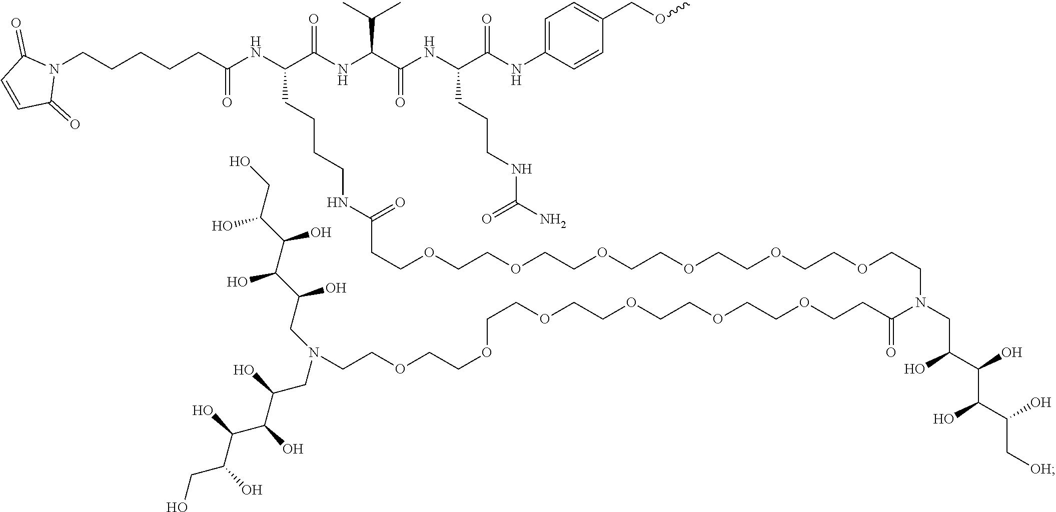

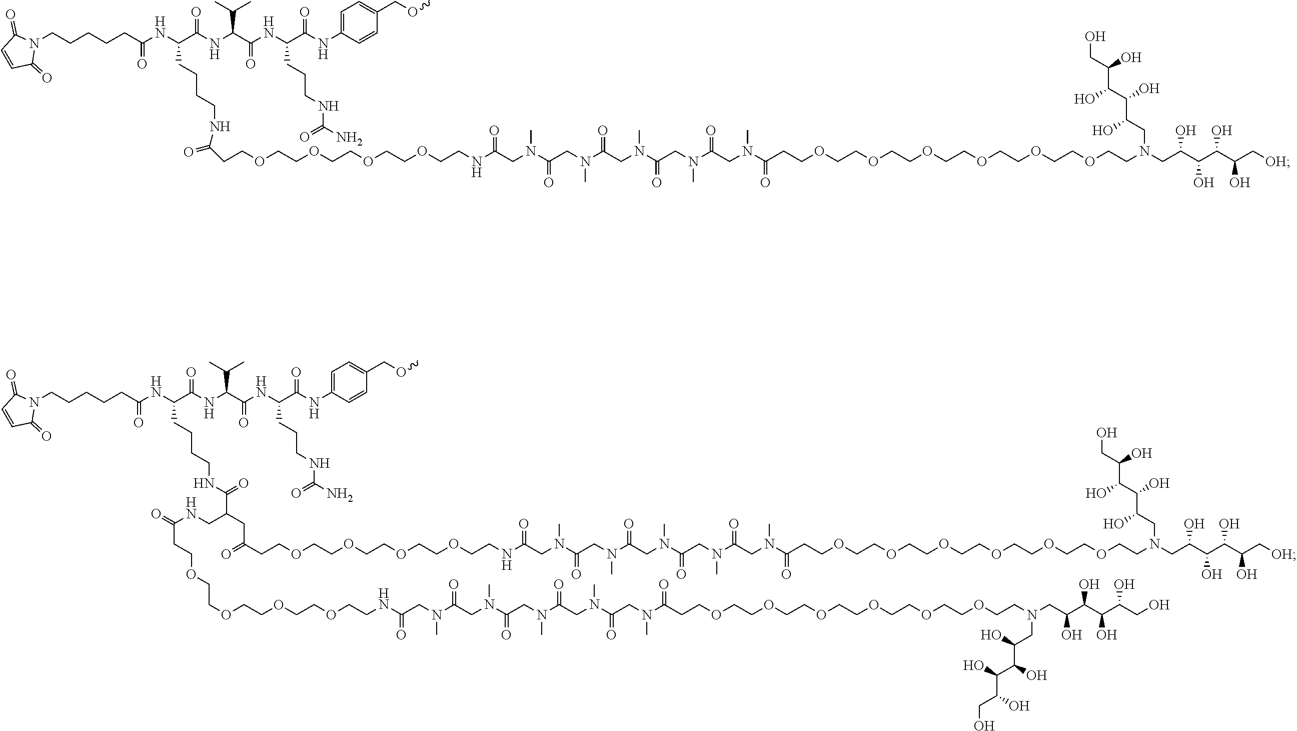

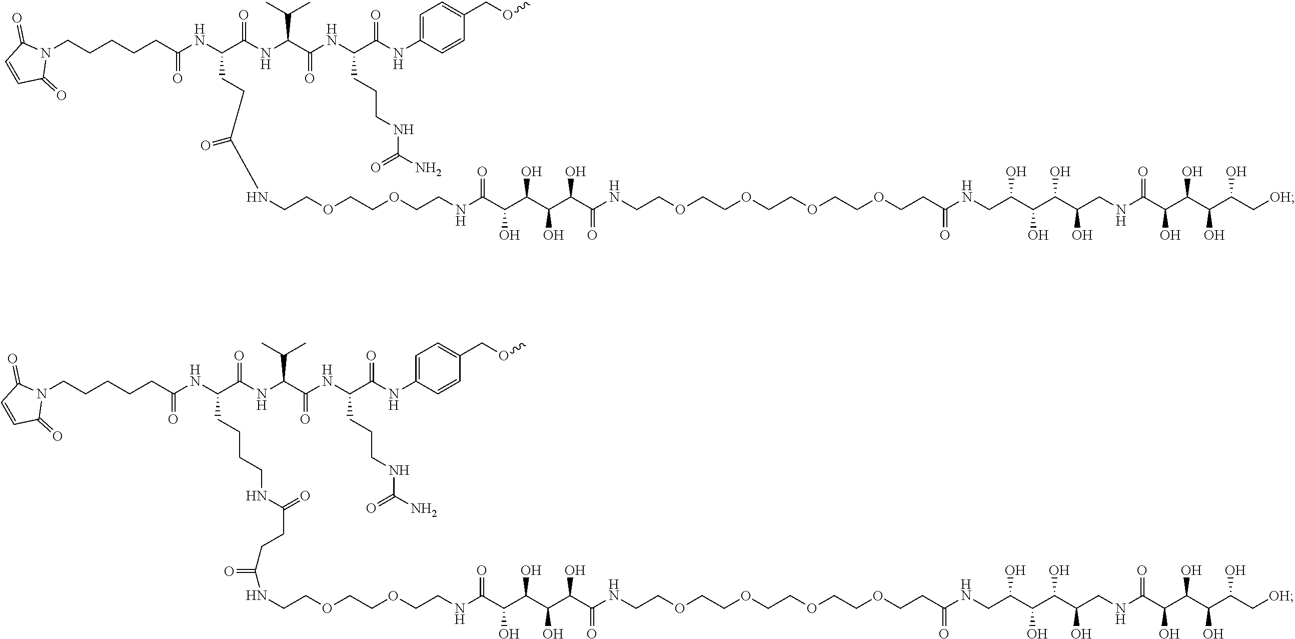

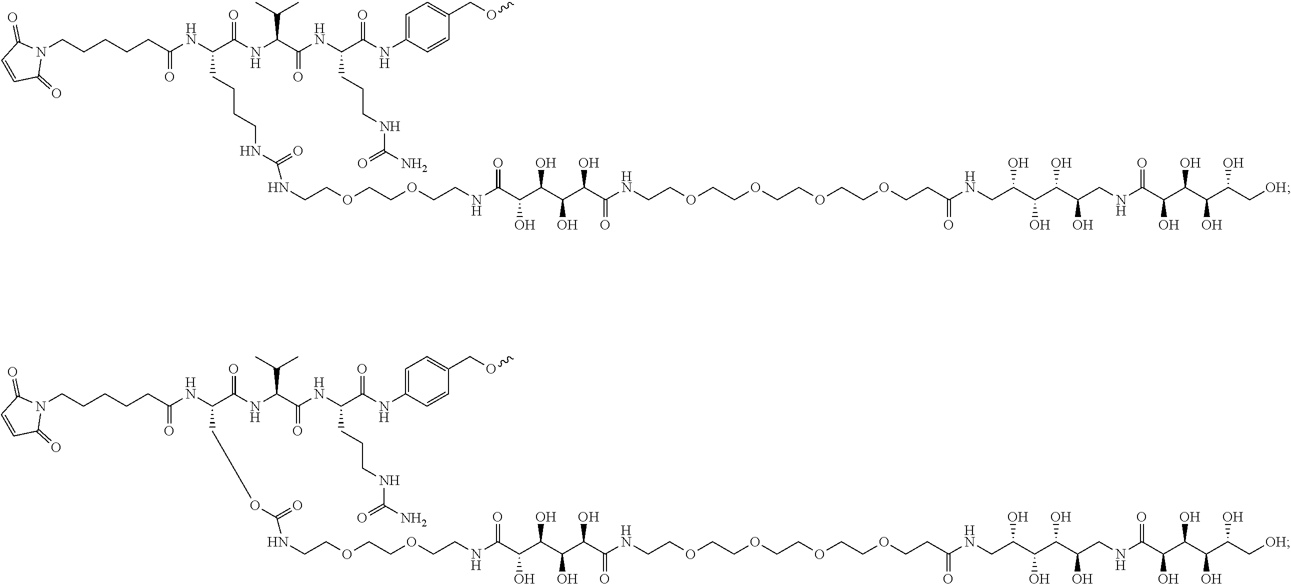

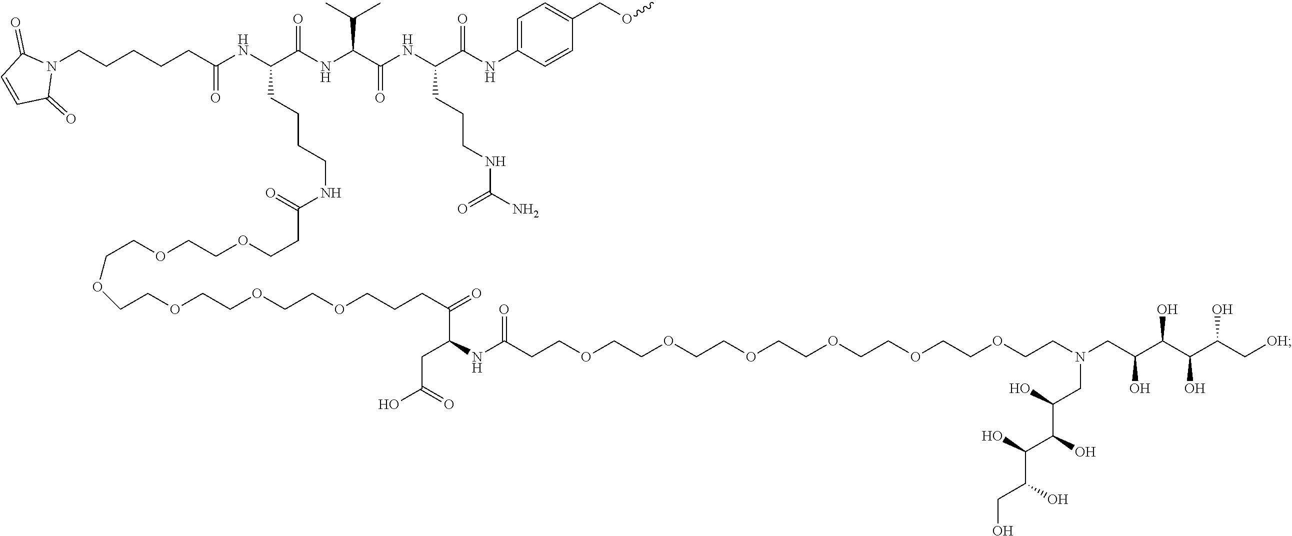

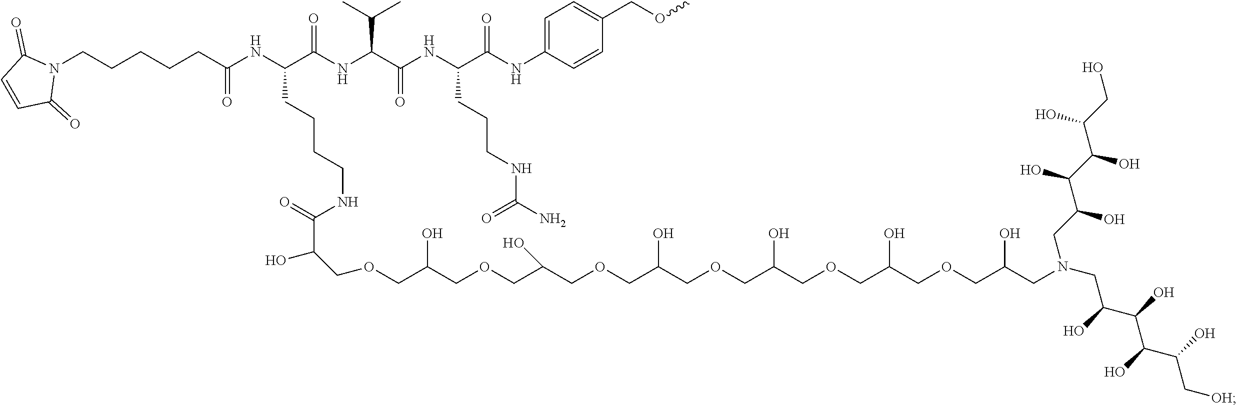

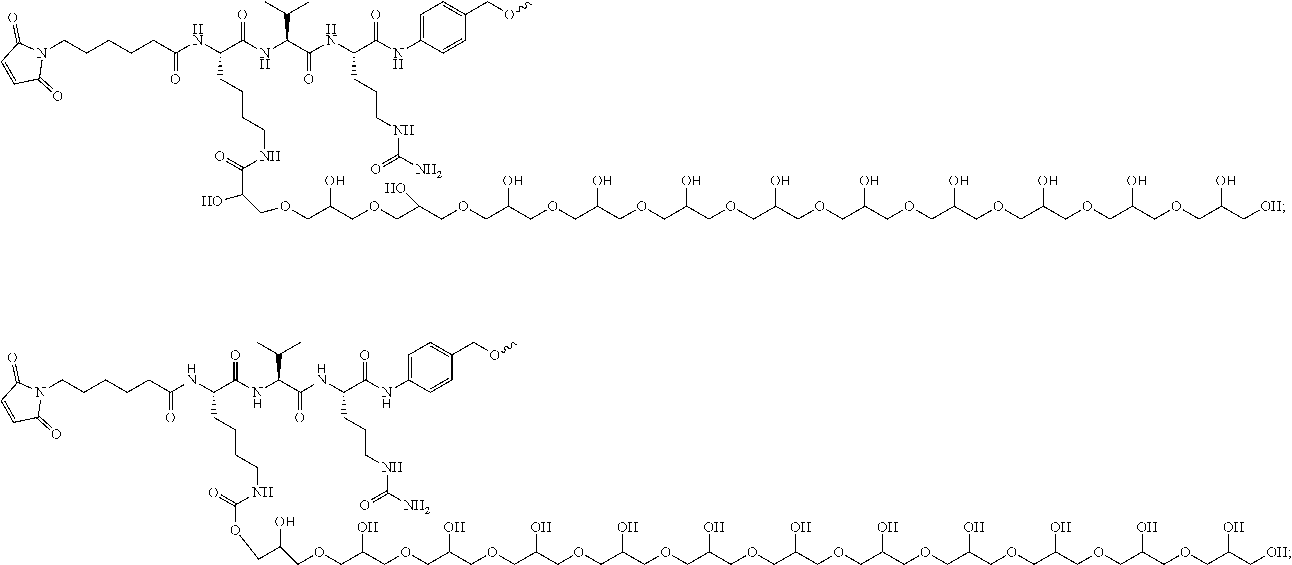

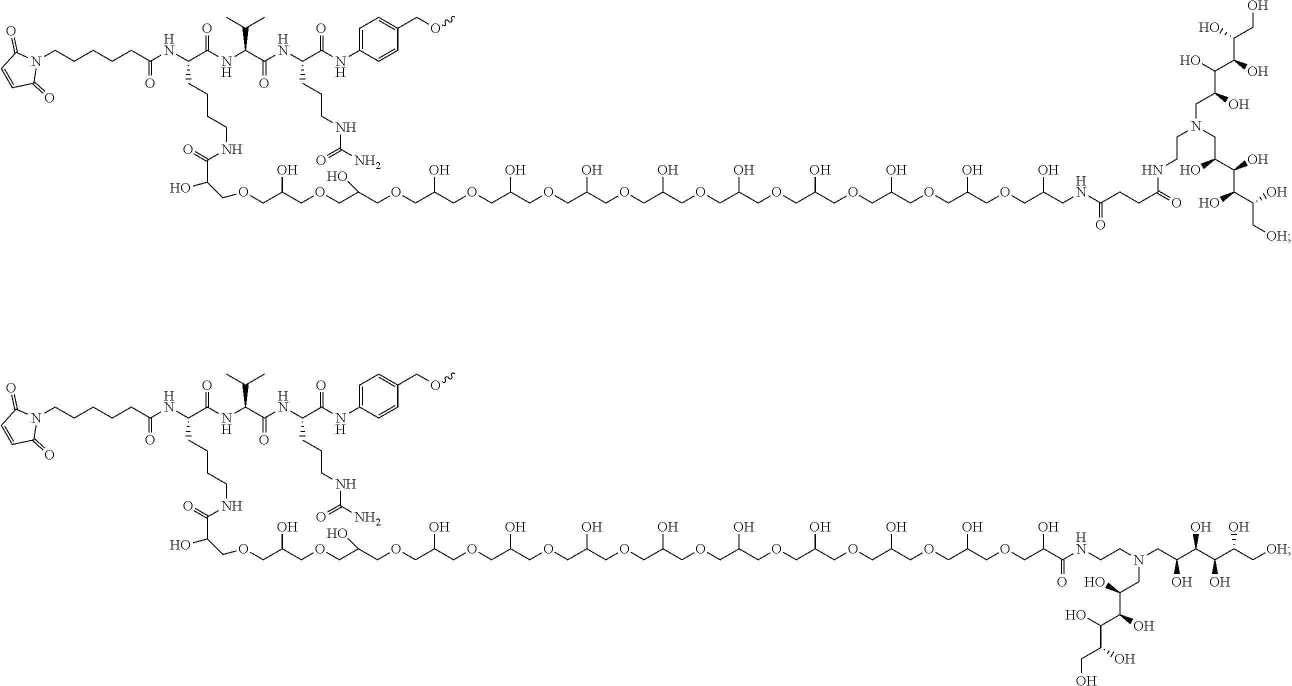

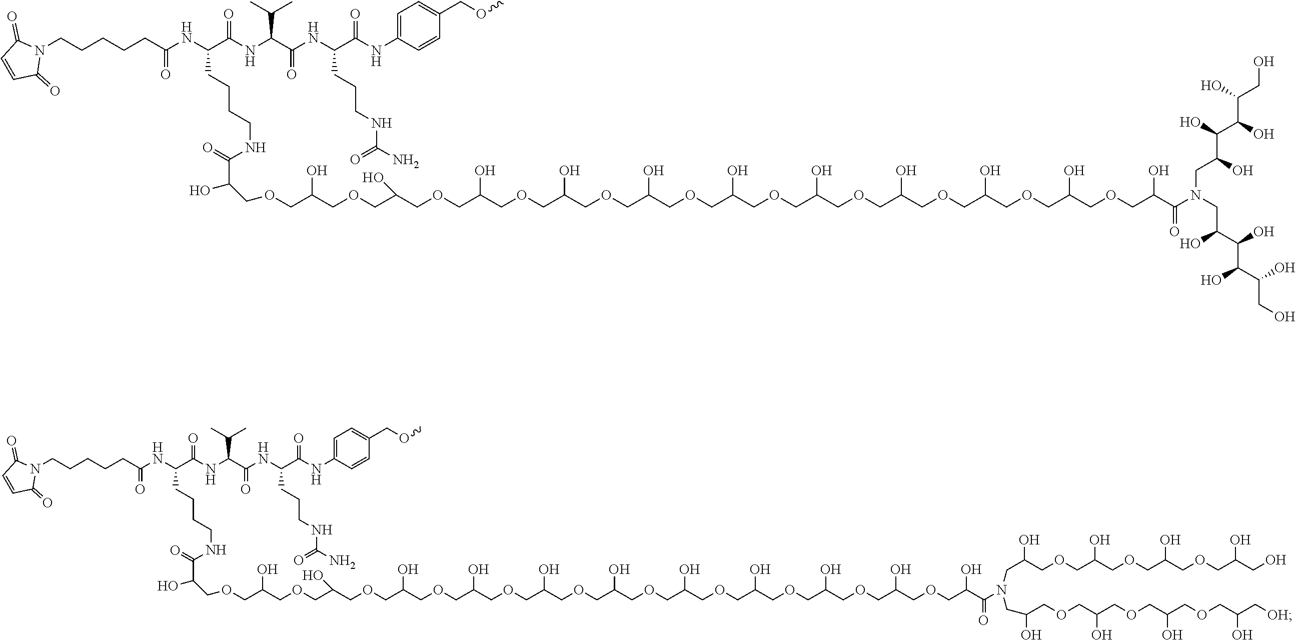

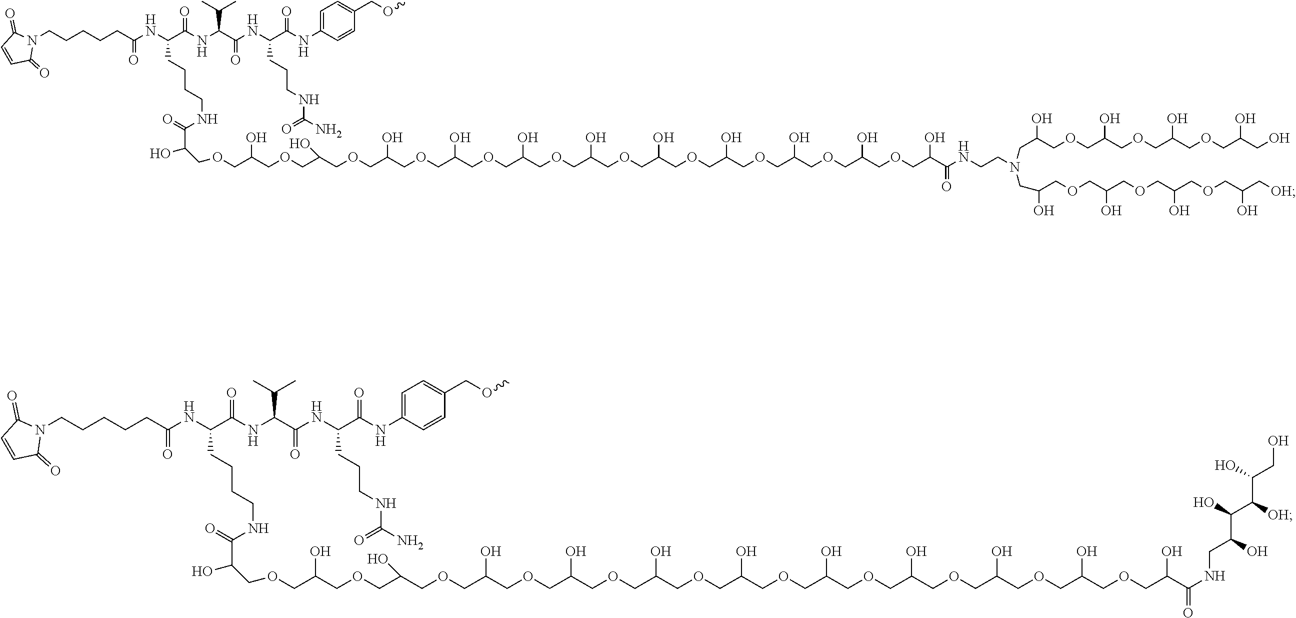

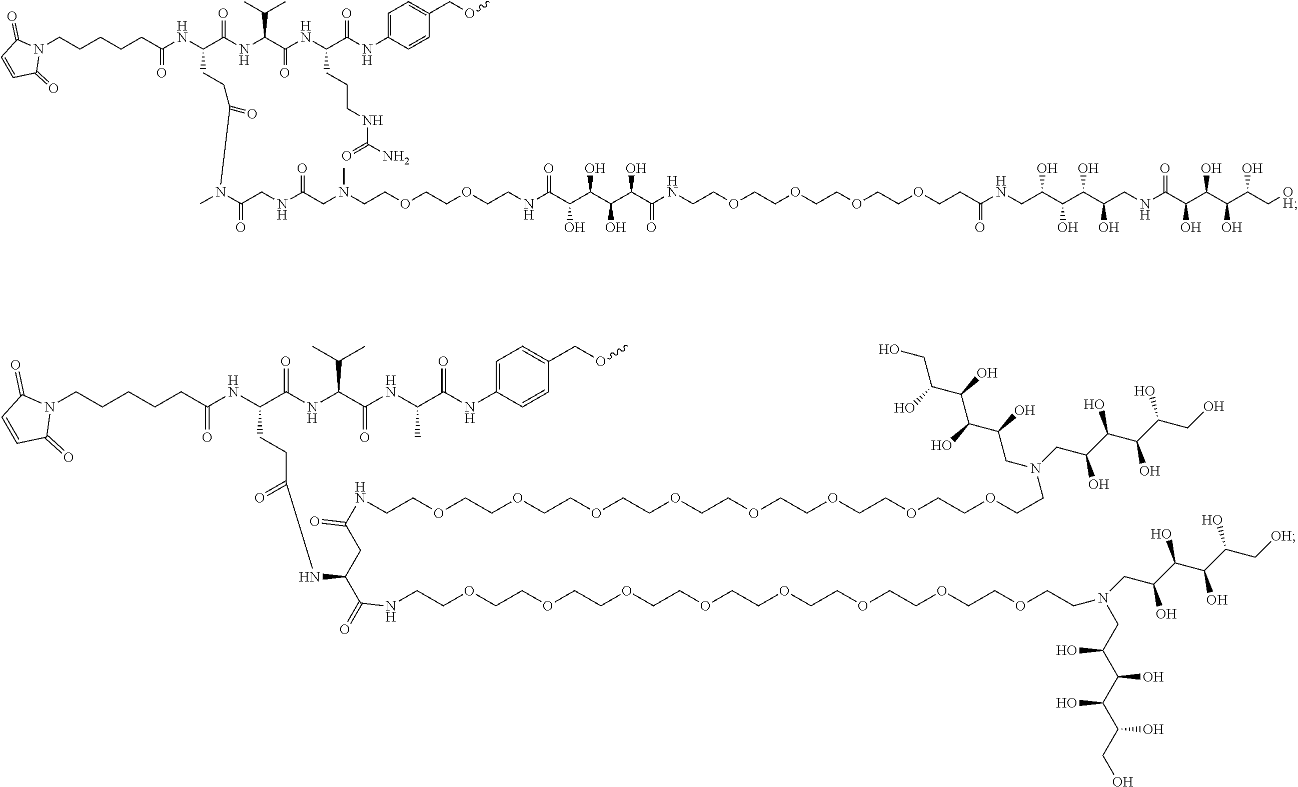

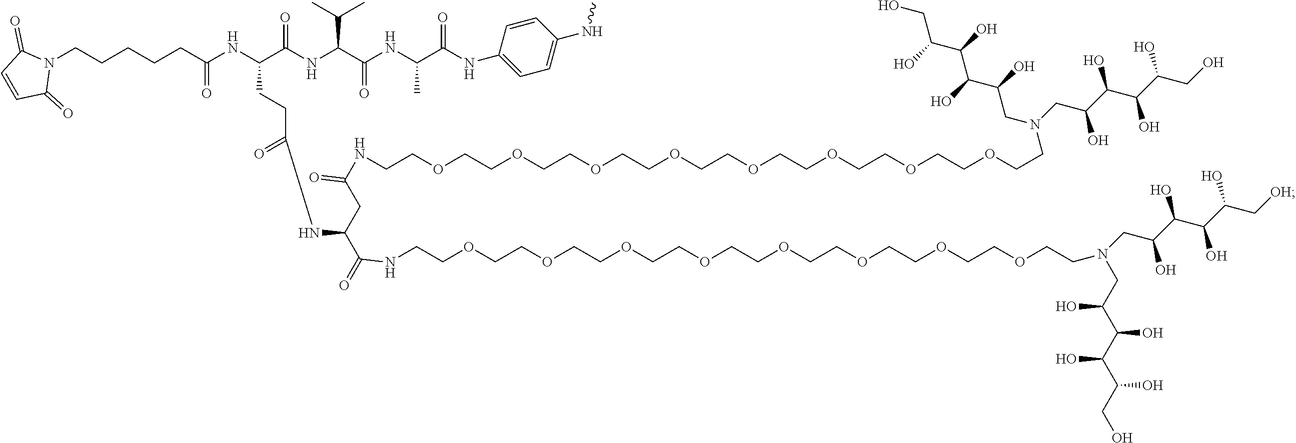

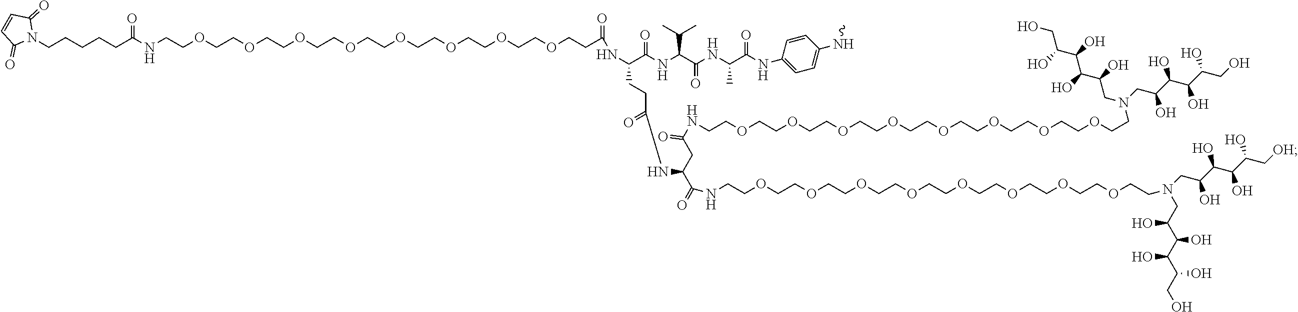

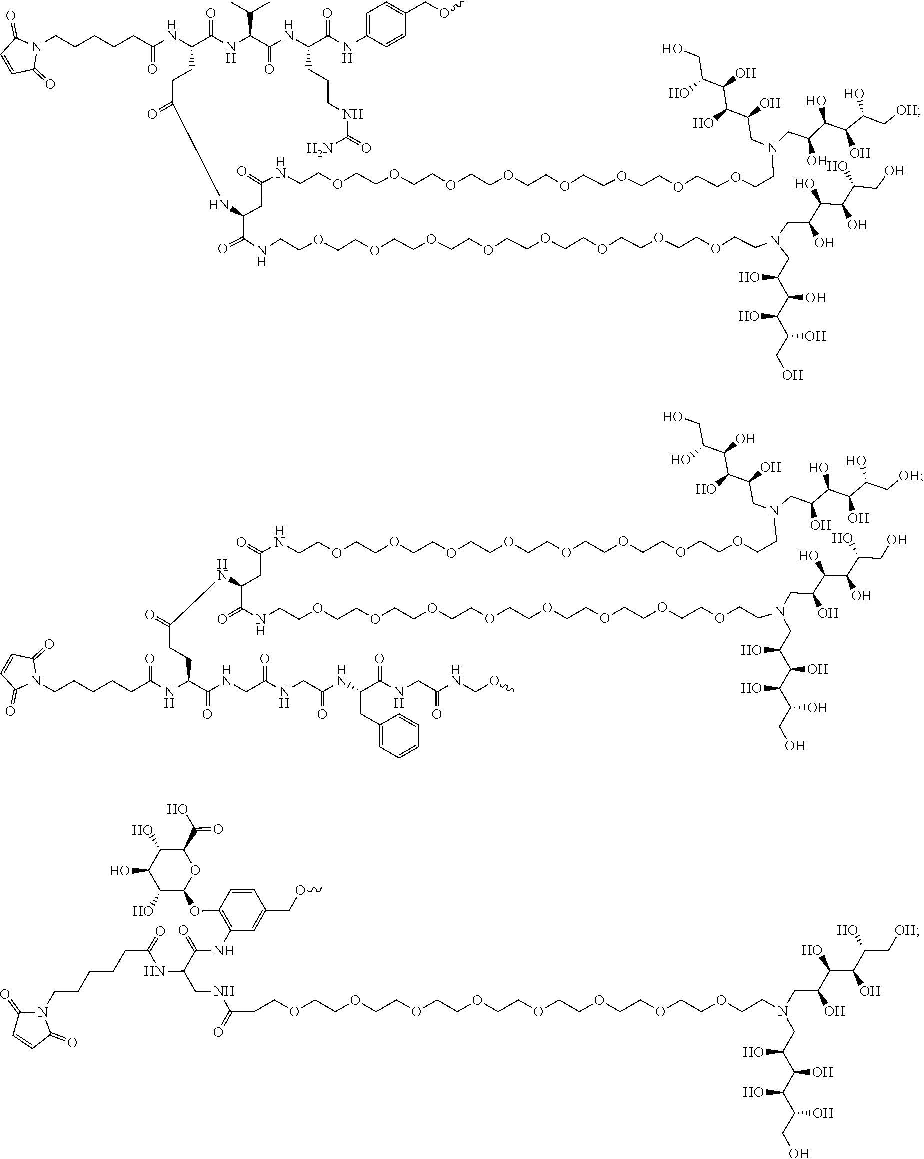

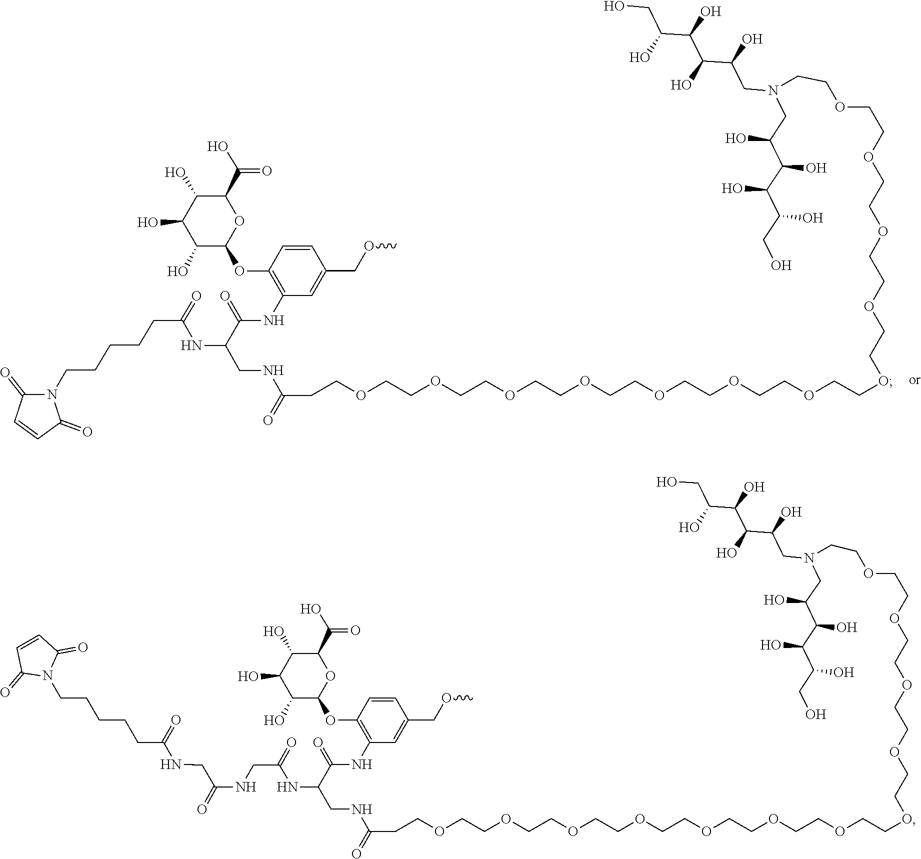

or a stereoisomer thereof, wherein the wavy line [symbol] indicates the attachment site to at least one of the drug units or for a linking group attached to the at a one of the drug units.

Embodiment 109. A drug-linker compound, comprising the linker compound of any one of Embodiments 1-108 attached to the at least one drug unit, or attached to a linking group attached to the at least one drug unit.

Embodiment 110. The drug-linker compound of Embodiment 109, wherein the drug unit is selected from a cytotoxic agent, an immune modulatory agent, a nucleic acid, a growth inhibitory agent, a PROTAC, a toxin, a radioactive isotope and a chelating ligand.

Embodiment 111. The drug-linker compound of Embodiment 111, wherein the drug unit is a cytotoxic agent.

Embodiment 112. The drug-linker compound of Embodiment 111, wherein the cytotoxic agent is selected from the group consisting of an auristatin, a maytansinoid, a camptothecin, a duocarmycin, and a calicheamicin.

Embodiment 113. The drug-linker compound of Embodiment 111, wherein the cytotoxic agent is an auristatin.

Embodiment 114. The drug-linker compound of Embodiment 113, wherein the cytotoxic agent is MMAE or MMAF

Embodiment 115. The drug-linker compound of Embodiment 111, wherein the cytotoxic agent is a camptothecin.

Embodiment 116. The drug-linker compound of Embodiment 115, wherein the cytotoxic agent is exatecan, or SN-38, or DxD.

Embodiment 117. The drug-linker compound of Embodiment 116, wherein the cytotoxic agent is RS-exatecan or SS-exatecan.

Embodiment 118. The drug-linker compound of Embodiment 111, wherein the cytotoxic agent is a calicheamicin.

Embodiment 119. The drug-linker compound of Embodiment 111, wherein the cytotoxic agent is a maytansinoid.

Embodiment 120. The drug-linker compound of Embodiment 119, wherein the maytansinoid is maytansine, maytansinol or ansamatocin-2.

Embodiment 121. The drug-linker of Embodiment 112, wherein the cytotoxic agent is MMAE, MMAF, exatecan, RS-exatecan, SS-exatecan, SN-38, DxD, maytansine, maytansinol or ansamatocin-2.

Embodiment 122. The drug-linker compound of Embodiment 112, wherein the cytotoxic agent is MMAE.

Embodiment 123. The drug-linker compound of Embodiment 112, wherein the cytotoxic agent is exatecan.

Embodiment 124. The drug-linker compound of Embodiment 110, wherein the drug unit is an immune modulatory agent.

Embodiment 125. The drug-linker compound of Embodiment 124, wherein the immune modulatory agent is selected from a TRL7 agonist, a TLR8 agonist, a STING agonist, or a RIG-1 agonist.

Embodiment 126. The drug-linker compound of Embodiment 125, wherein the immune modulatory agent is an TLR7 agonist.

Embodiment 127. The drug-linker compound of Embodiment 126, wherein the TLR7 agonist is an imidazoquinoline, an imidazoquinoline amine, a thiazoquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine, heteroarothiadiazide-2,2-dioxide, a benzonaphthyridine, a guanosine analog, an adenosine analog, a thymidine homopolymer, ssRNA, CpG-A, PolyG10, or PolyG3.

Embodiment 128. The drug-linker compound of Embodiment 126, wherein the immune modulatory agent is a TLR8 agonist.

Embodiment 129. The drug-linker compound of Embodiment 128, wherein the TLR8 agonist is selected from an imidazoquinoline, a thiazoloquinoline, an aminoquinoline, an aminoquinazoline, a pyrido [3,2-d]pyrimidine-2,4-diamine, pyrimidine-2,4-diamine, 2-aminoimidazole, 1-alkyl-1H-benzimidazol-2-amine, tetrahydropyridopyrimidine or a ssRNA.

Embodiment 130. The drug-linker compound of Embodiment 125, wherein the immune modulatory agent is a STING agonist.

Embodiment 131. The drug-linker compound of Embodiment 125, wherein the immune modulatory agent is a RIG-1 agonist.

Embodiment 132. The drug-linker compound of Embodiment 131, wherein the RIG-1 agonist is selected from KIN1148, SB-9200, KIN700, KIN600, KIN500, KIN100, KIN101, KIN400 and KIN2000.

Embodiment 133. The drug-linker compound of Embodiment 110, wherein the drug unit is a chelating ligand.

Embodiment 134. The drug-linker compound of Embodiment 133, wherein the chelating ligand is selected from platinum (Pt), ruthenium (Ru), rhodium (Rh), gold (Au), silver (Ag), copper (Cu), molybdenum (Mo), titanium (Ti), or iridum (Ir); a radioisotope such as yittrium-88, yittrium-90, technetium-99, copper-67, rhenium-188, rhenium-186, galium-66, galium-67, indium-111, indium-114, indium-115, lutetium-177, strontium-89, sararium-153, and lead-212.

Embodiment 135. The drug-linker compound of Embodiment 109, having one of the following structure:

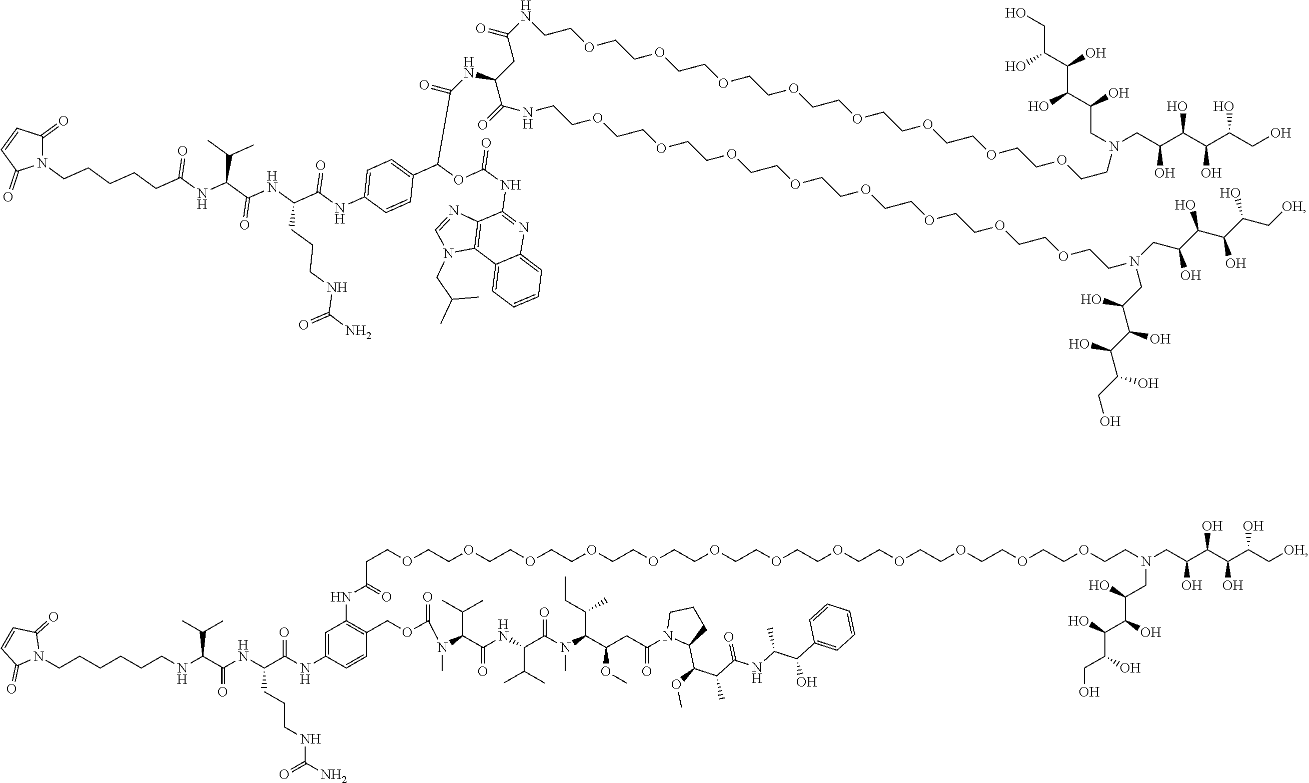

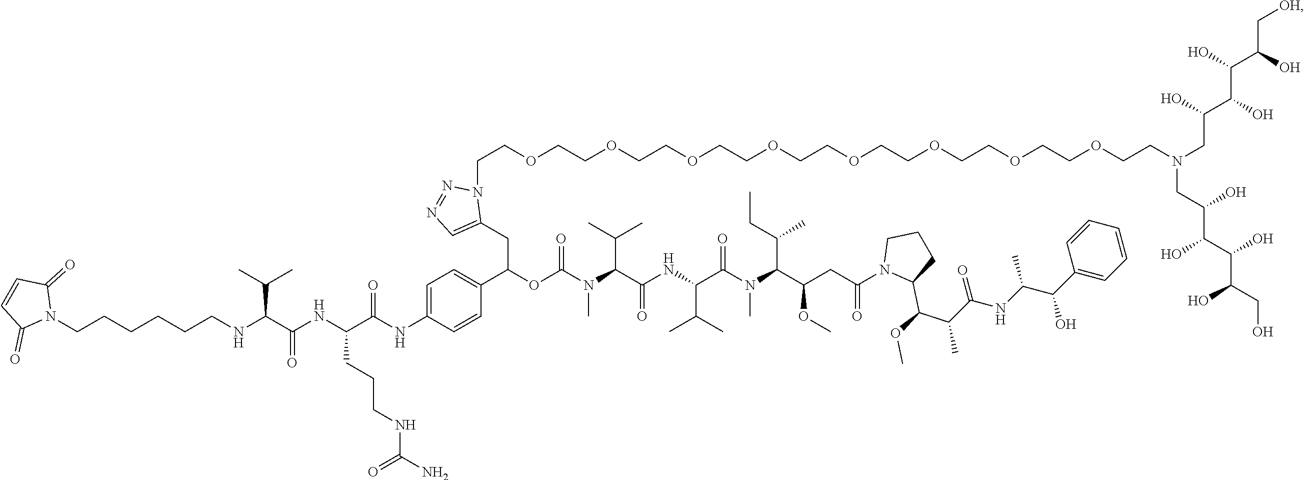 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

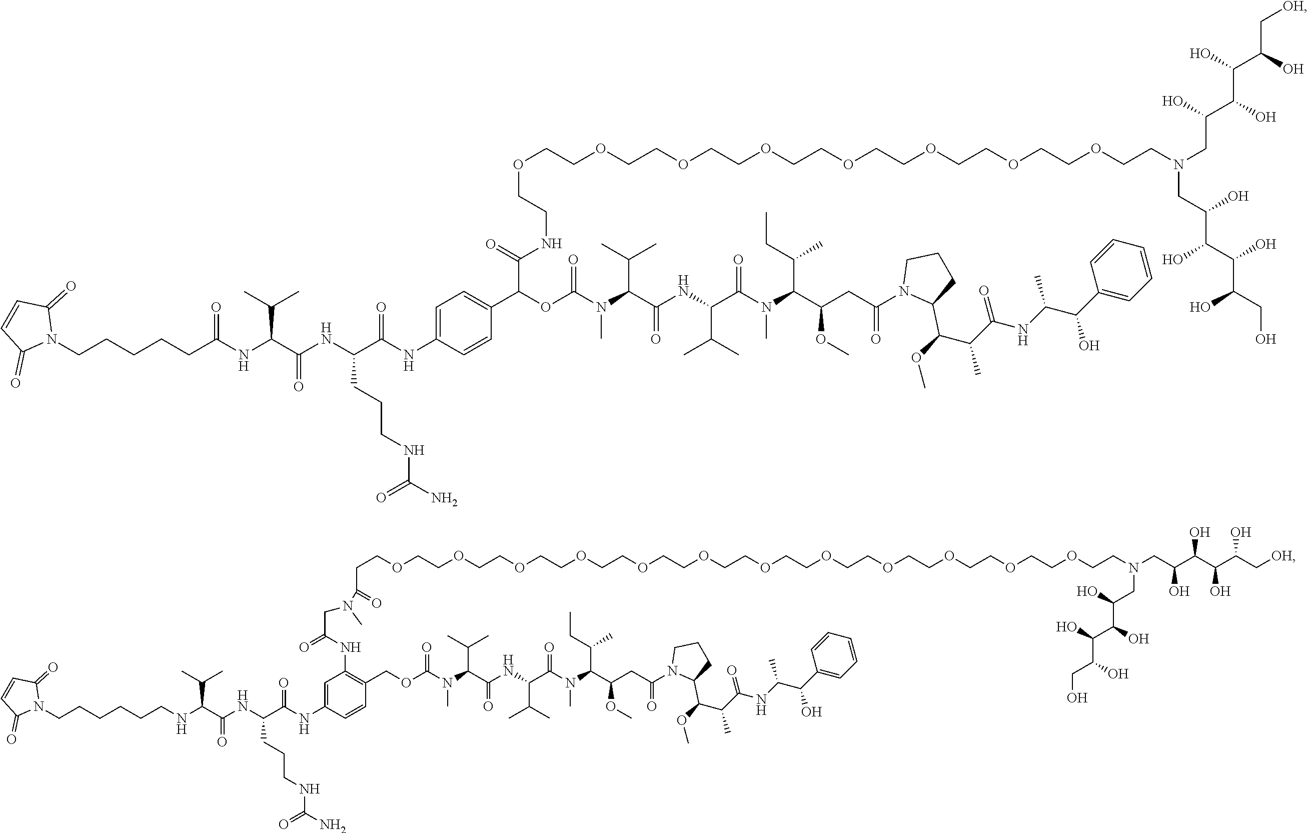

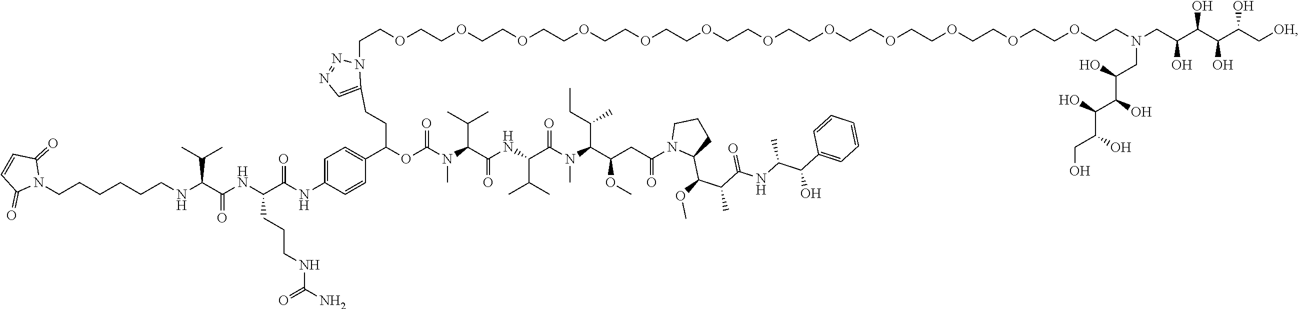 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(CCc1cnnn1CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

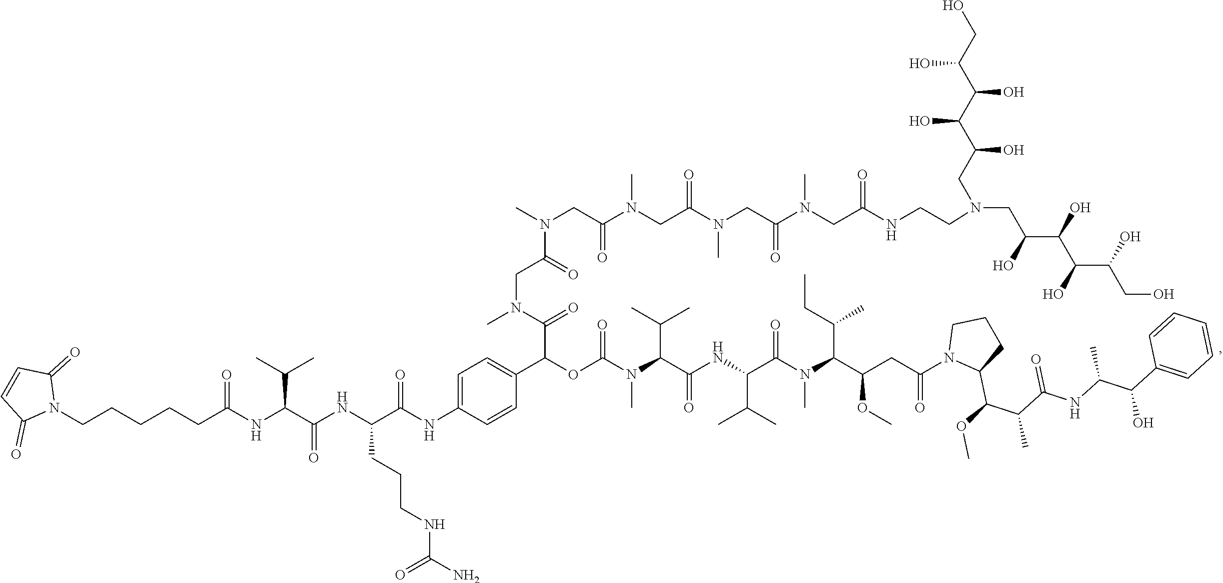 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

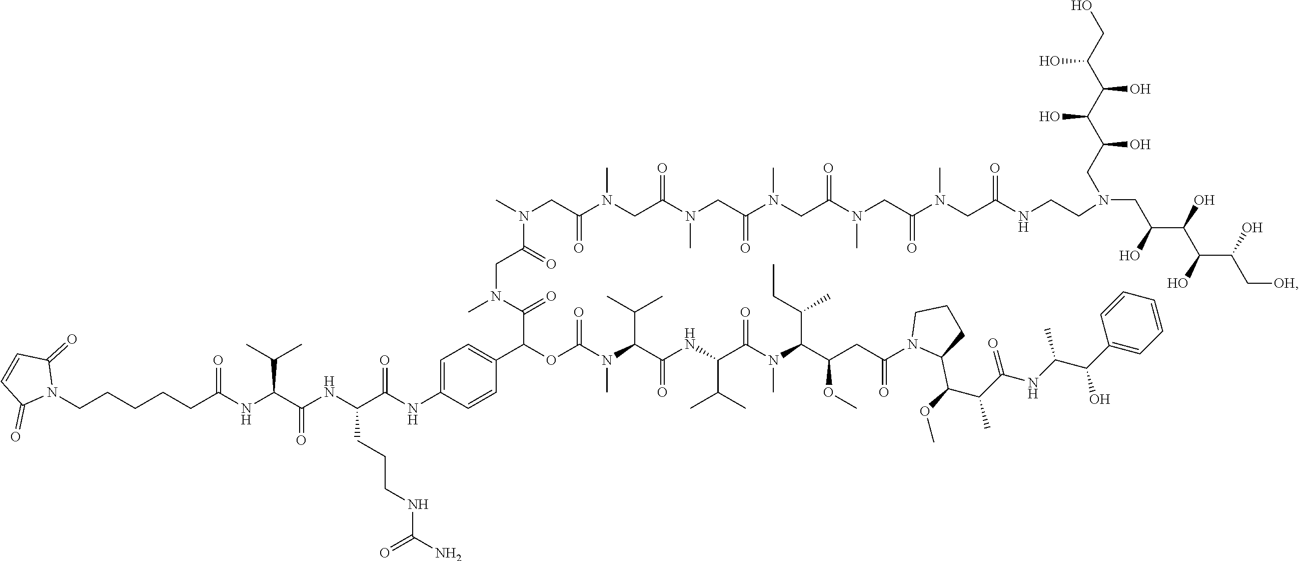 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

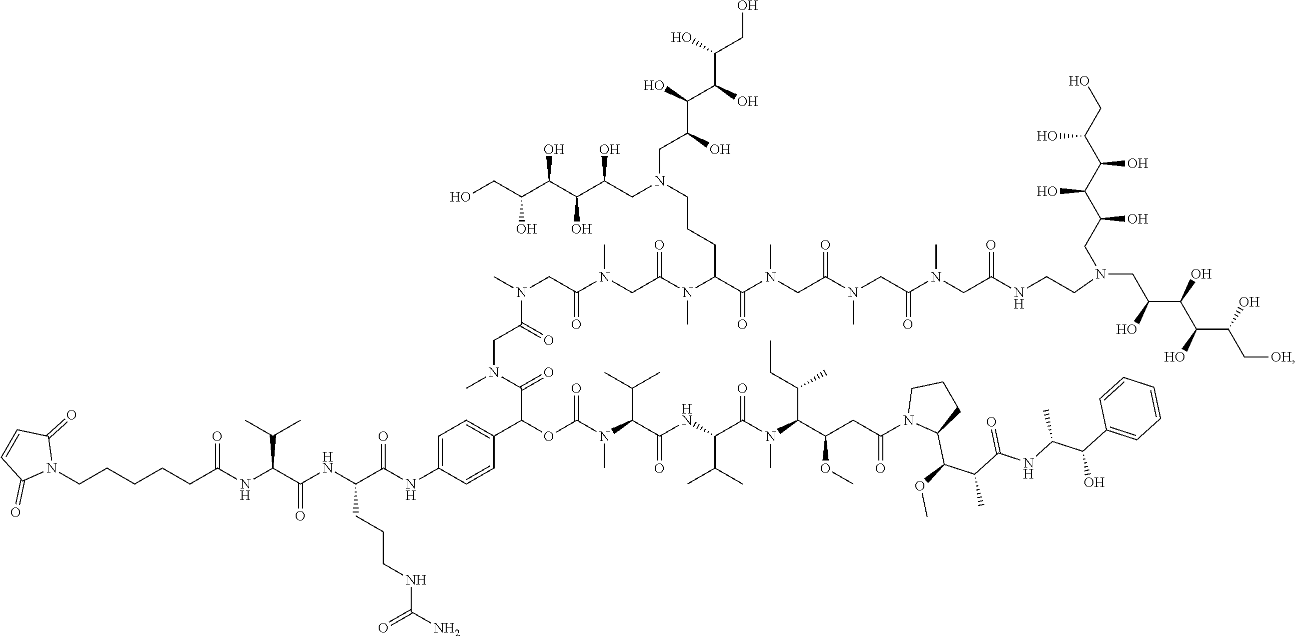 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

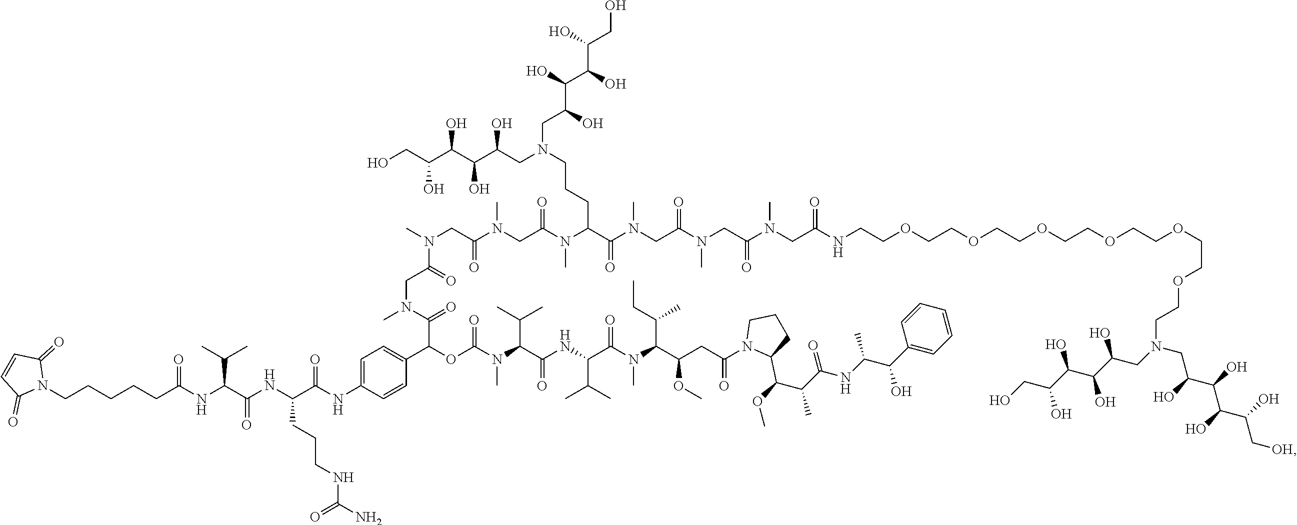 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

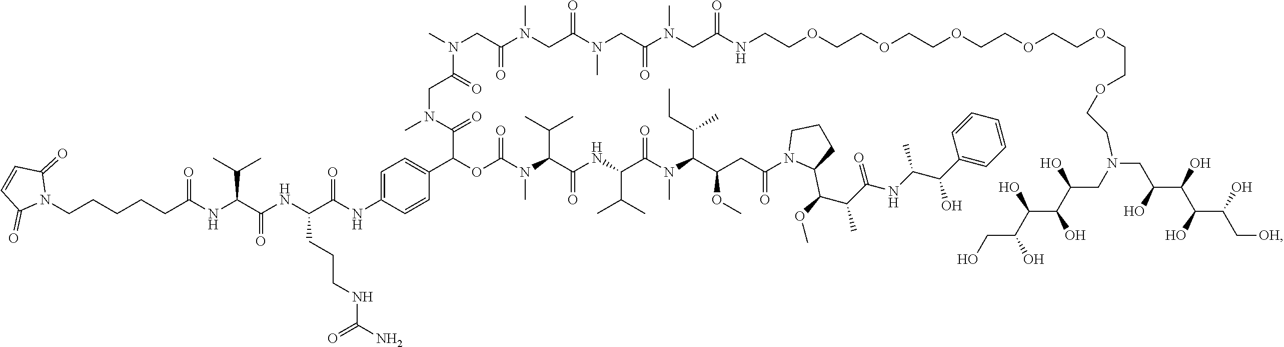 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

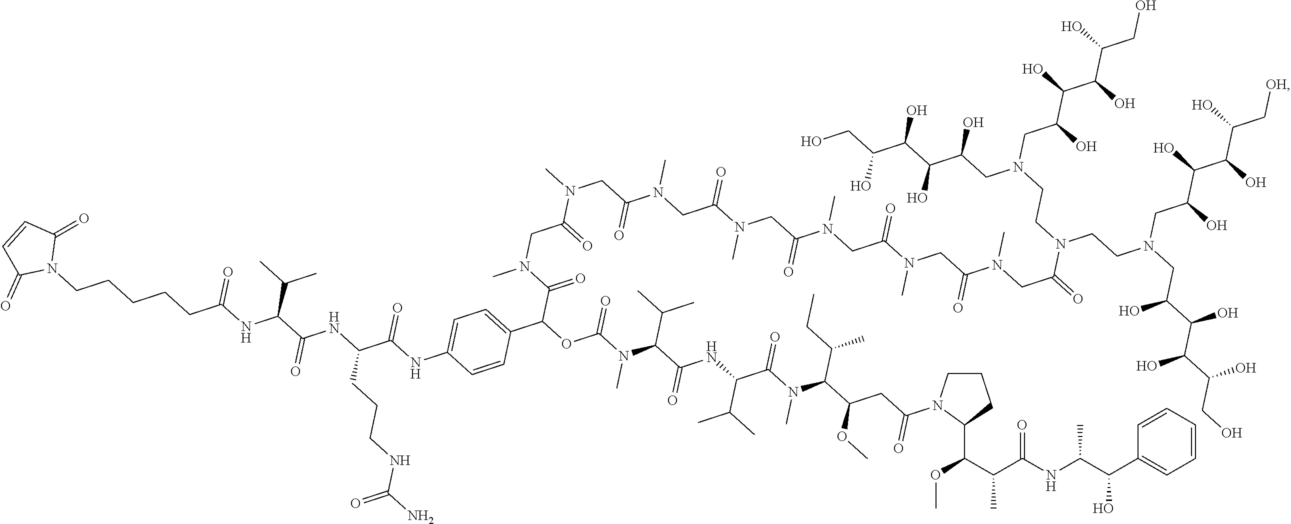 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

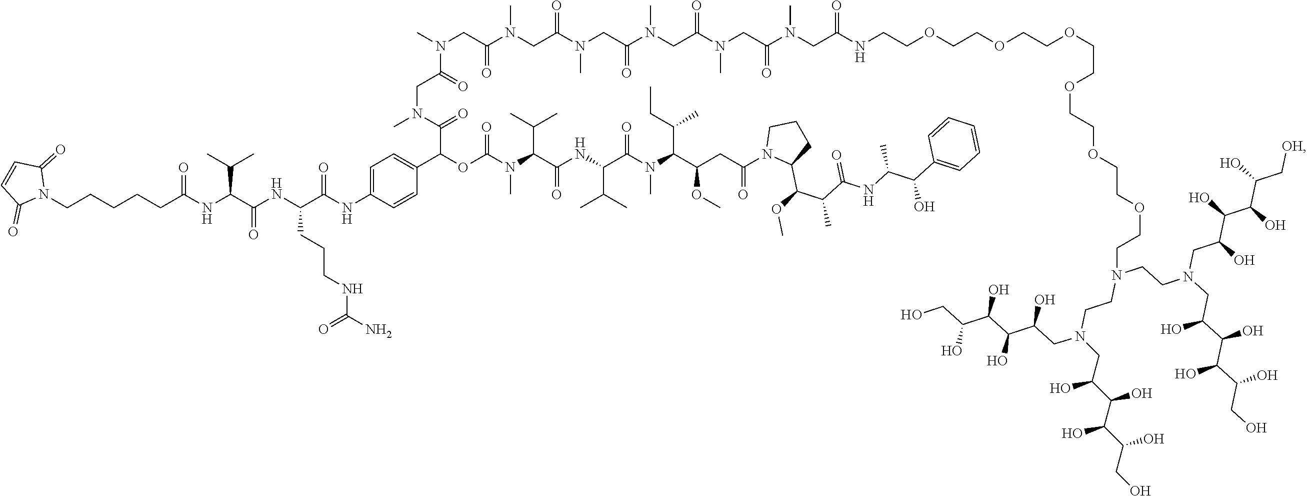 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

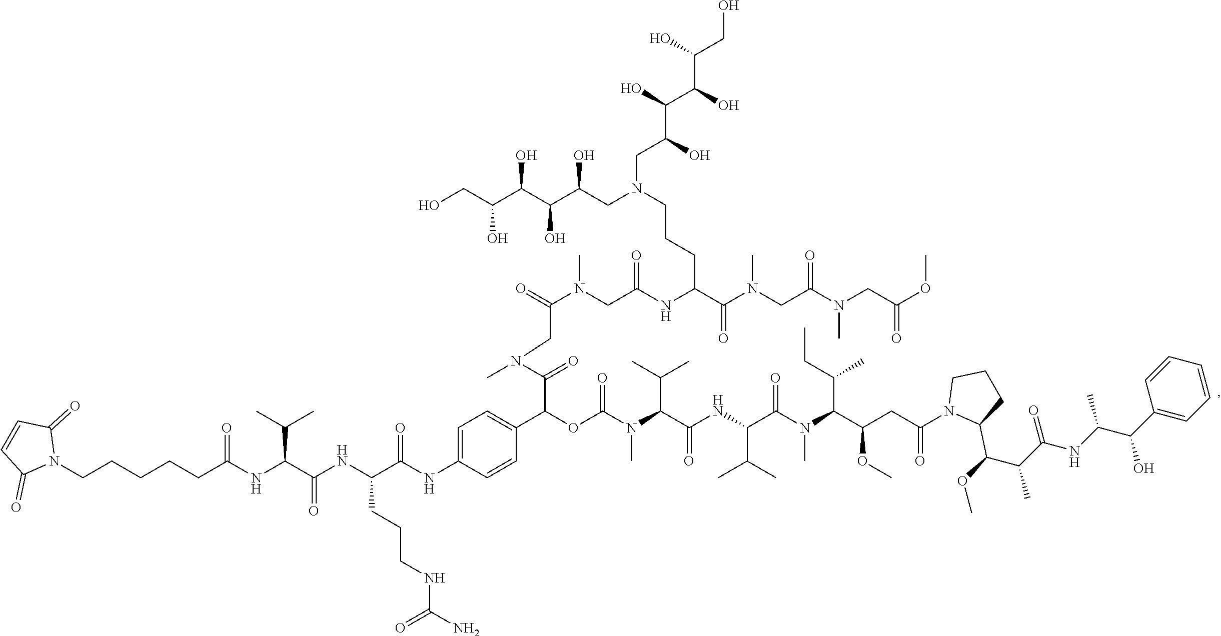 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

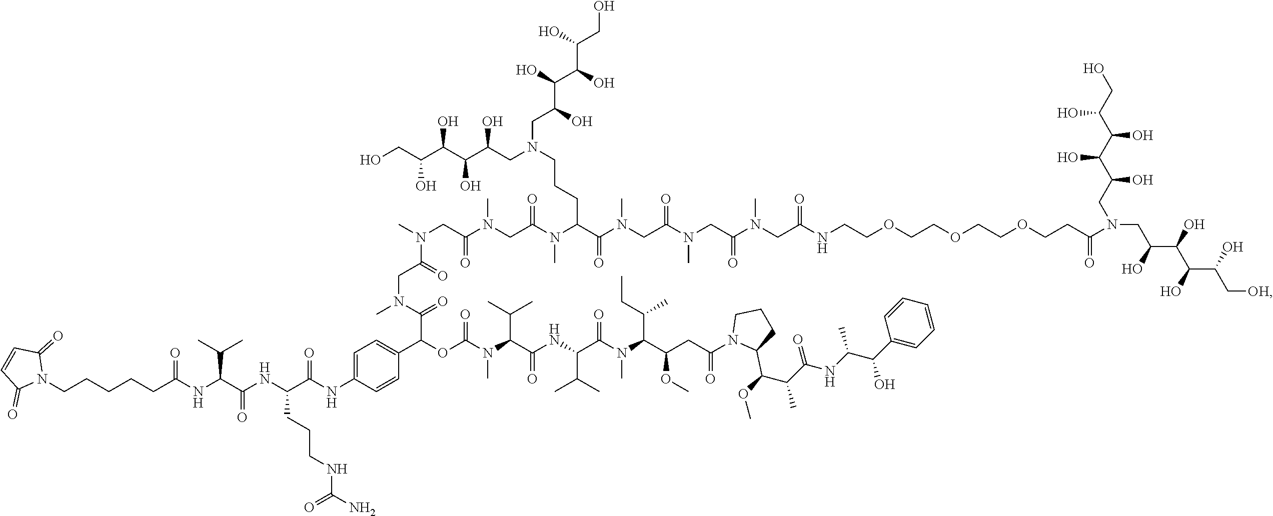 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

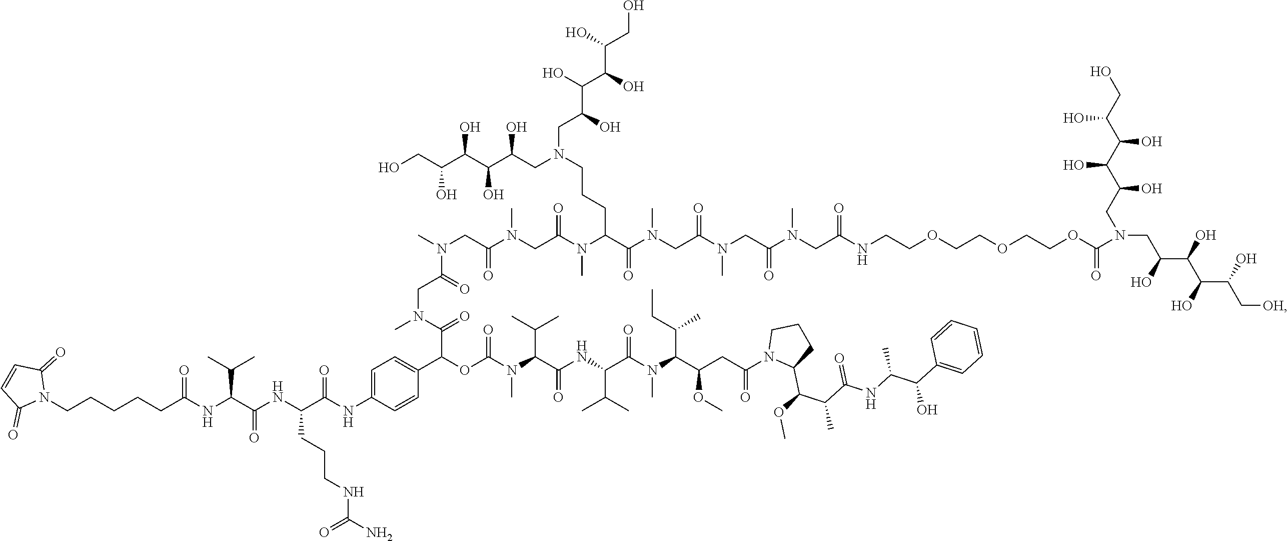 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)C(CCCN(C[C@H](O)C(O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

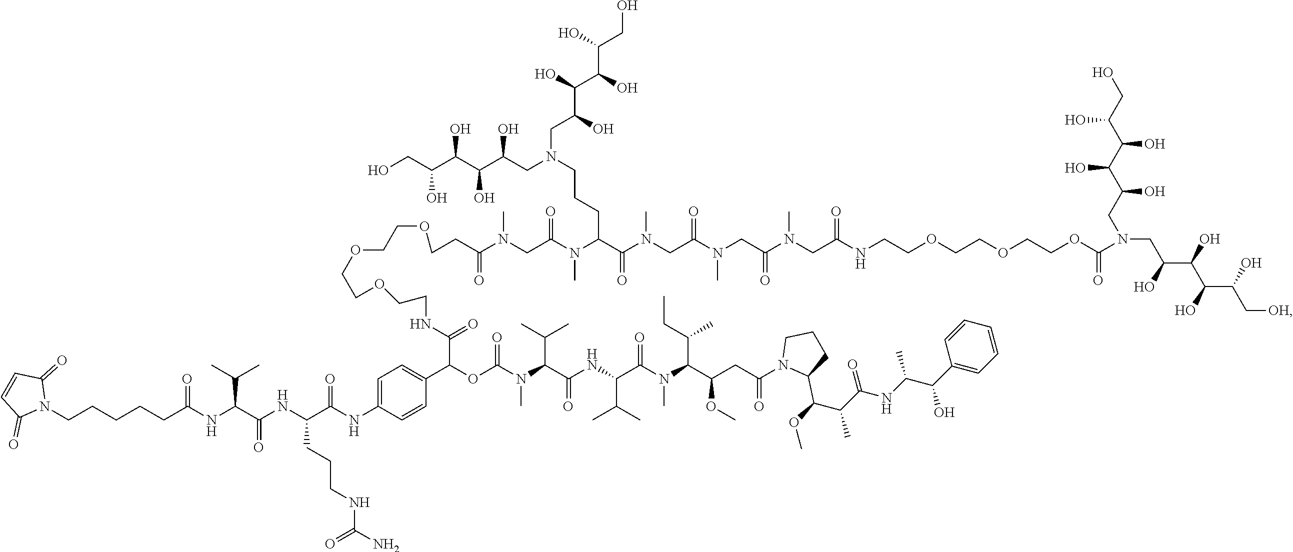 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCCOCCOCCOCCC(=O)N(C)CC(=O)N(C)C(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

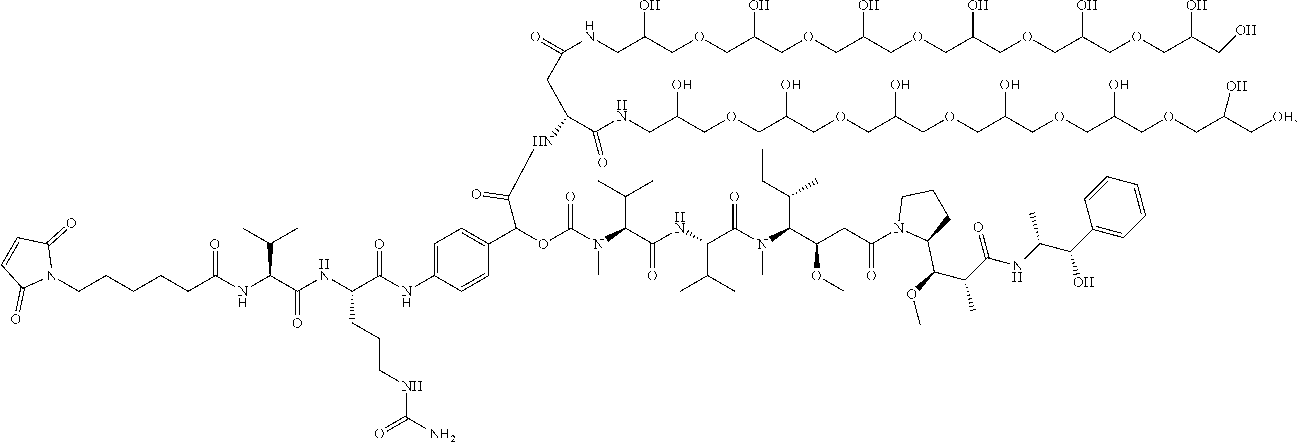 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

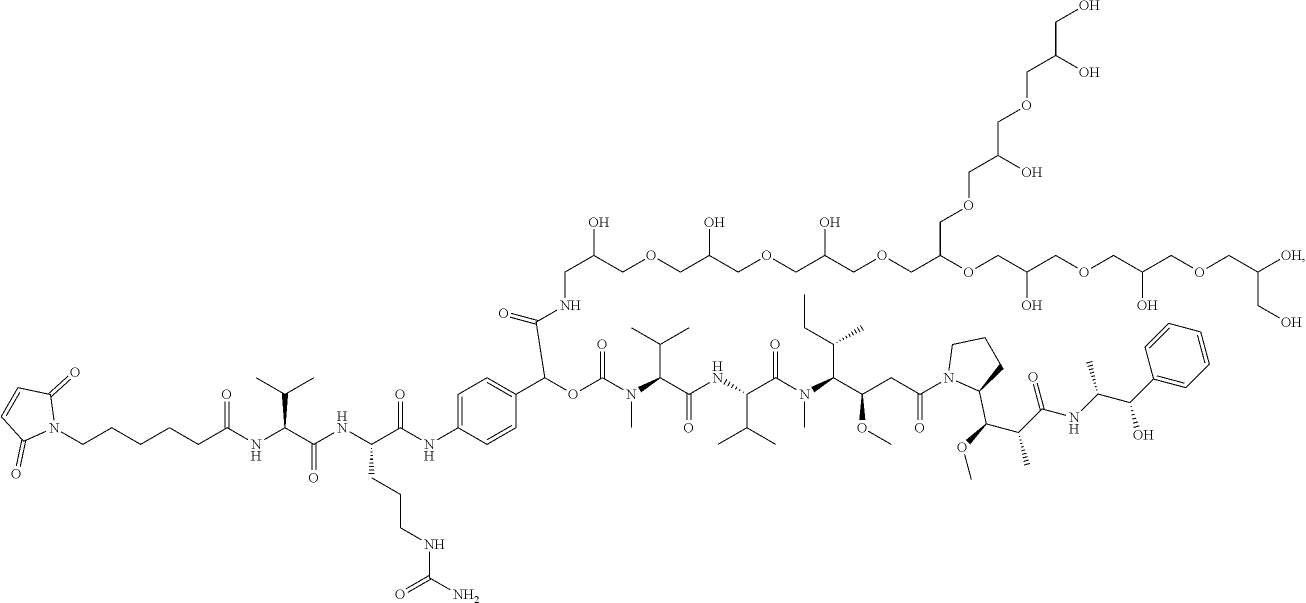 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(COCC(O)COCC(O)CO)OCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

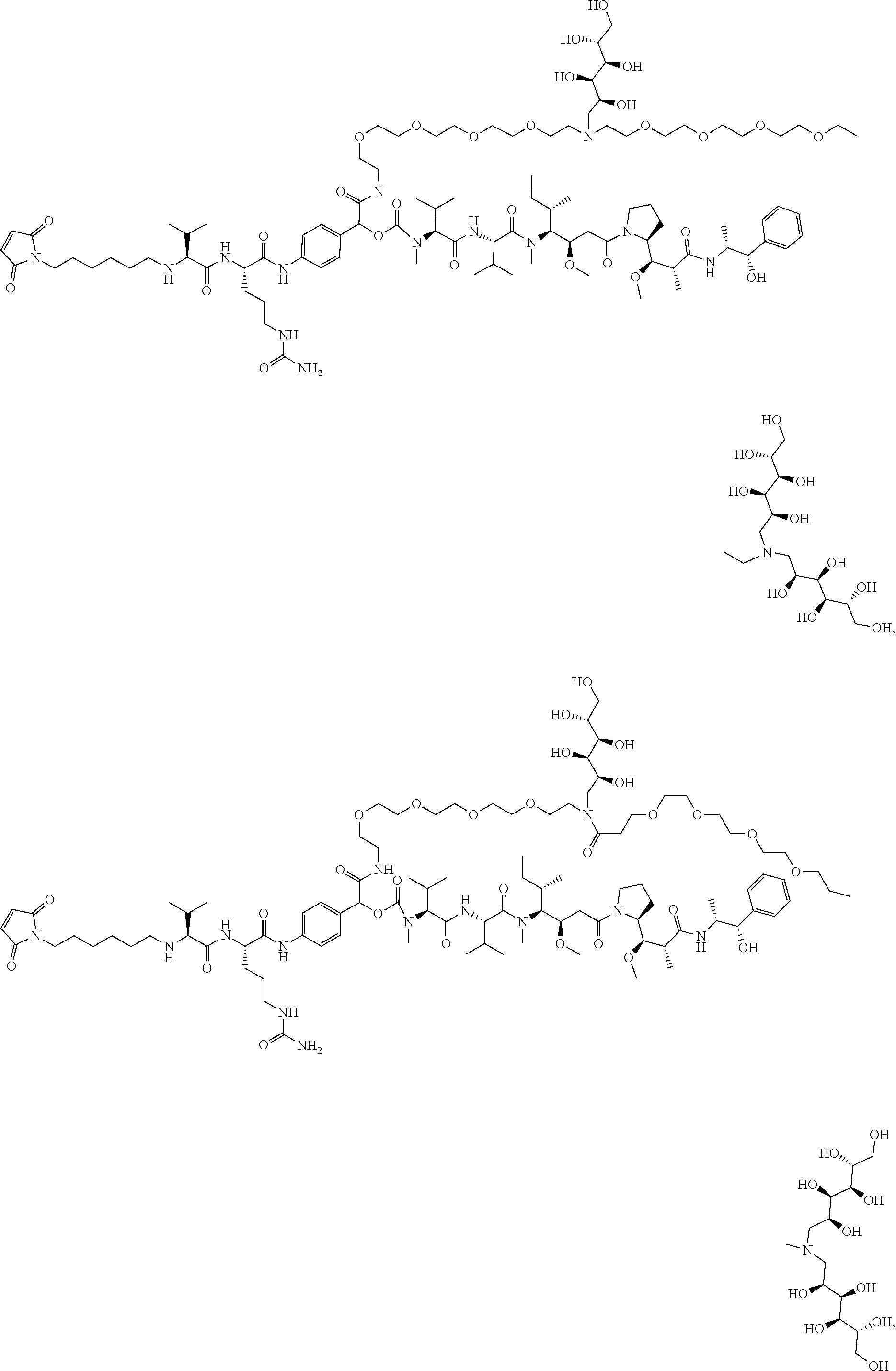

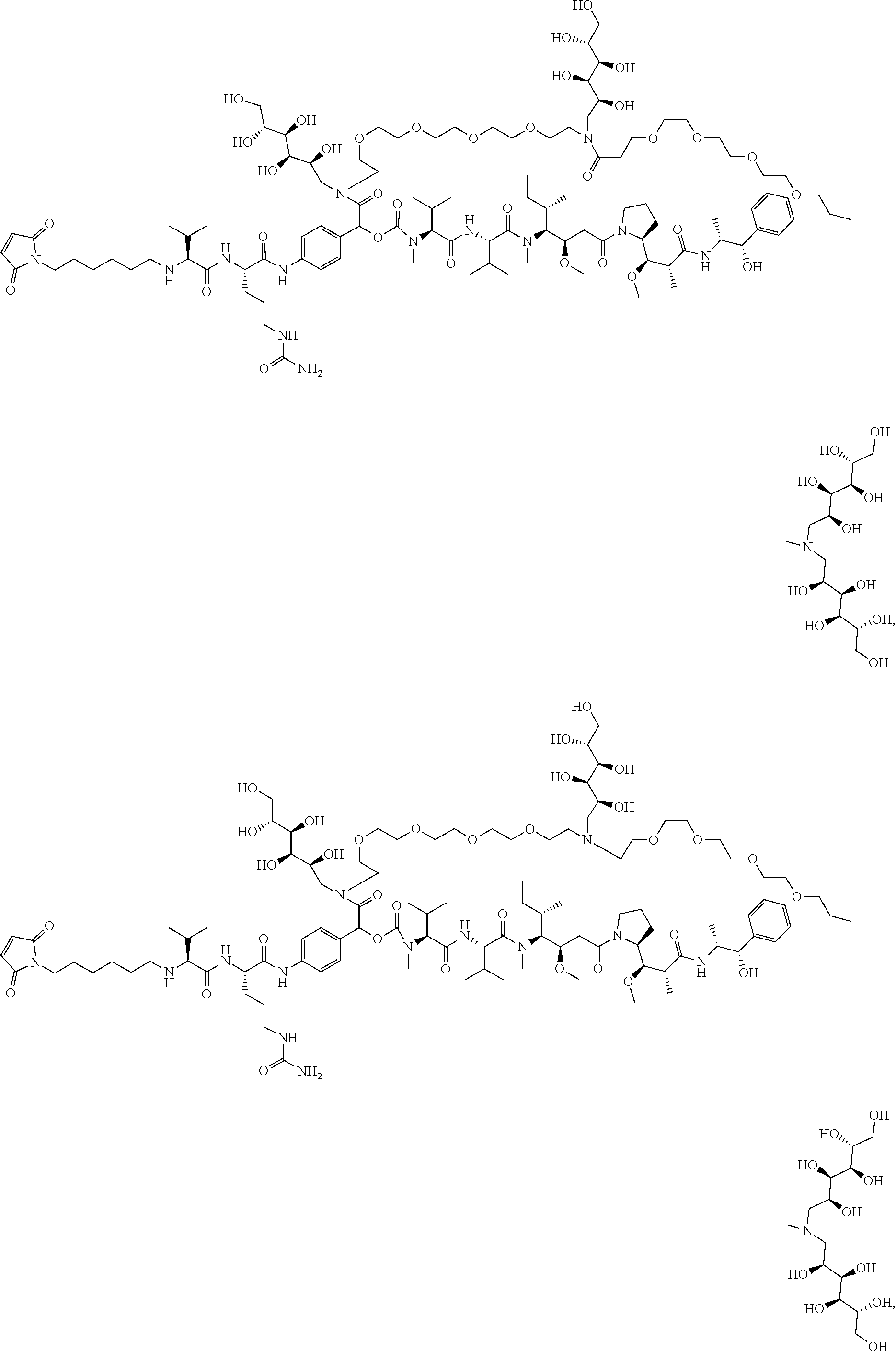

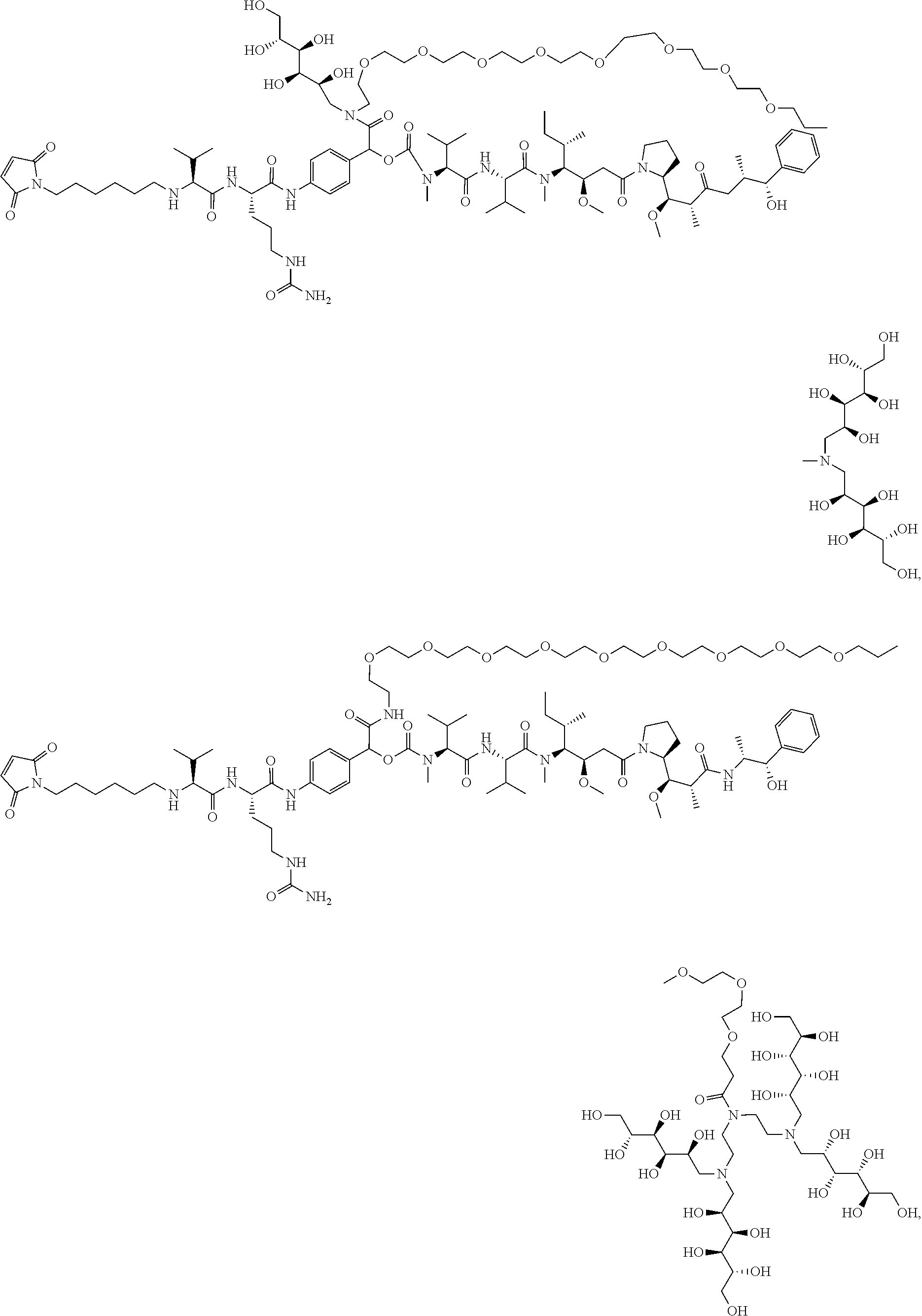

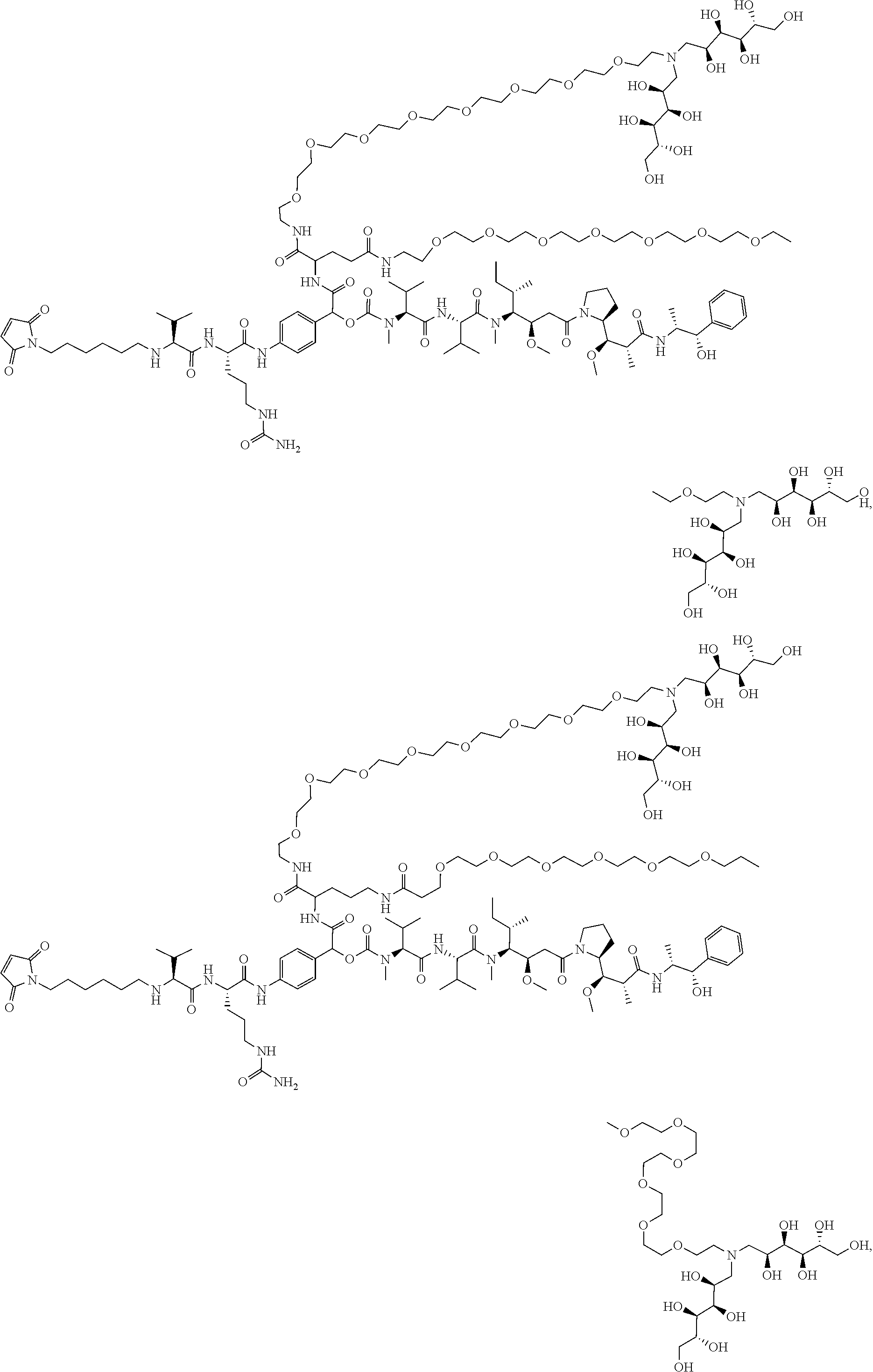

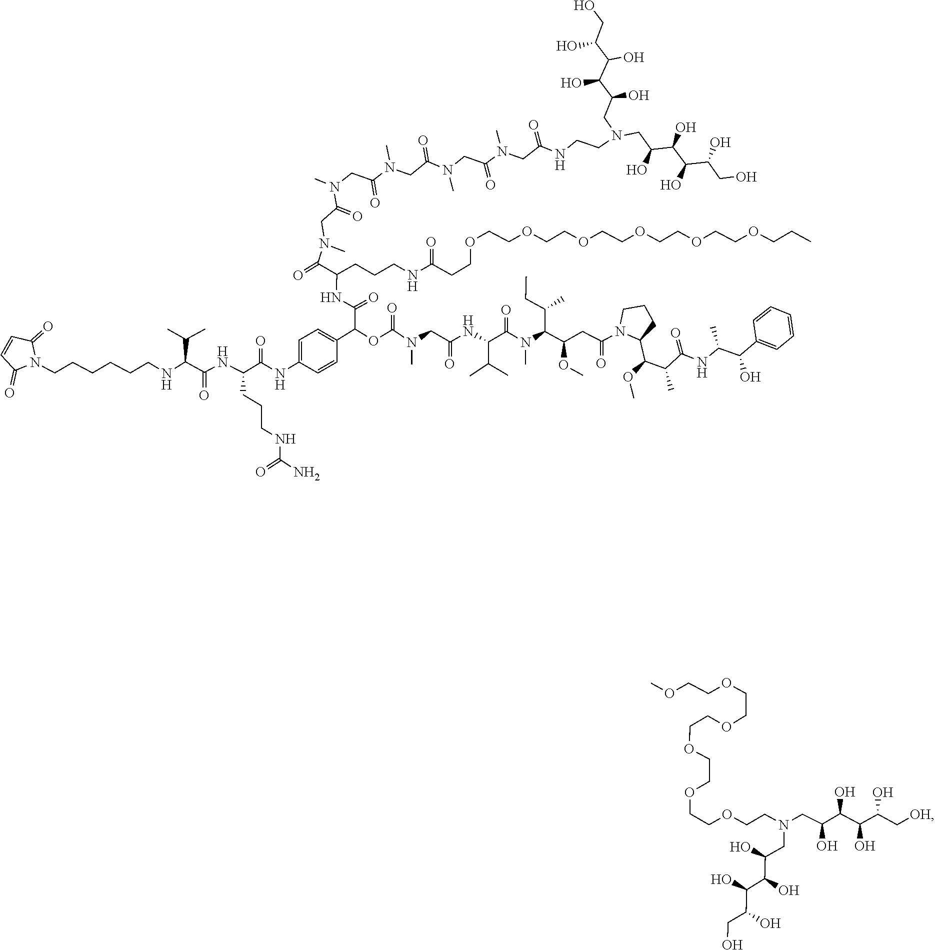

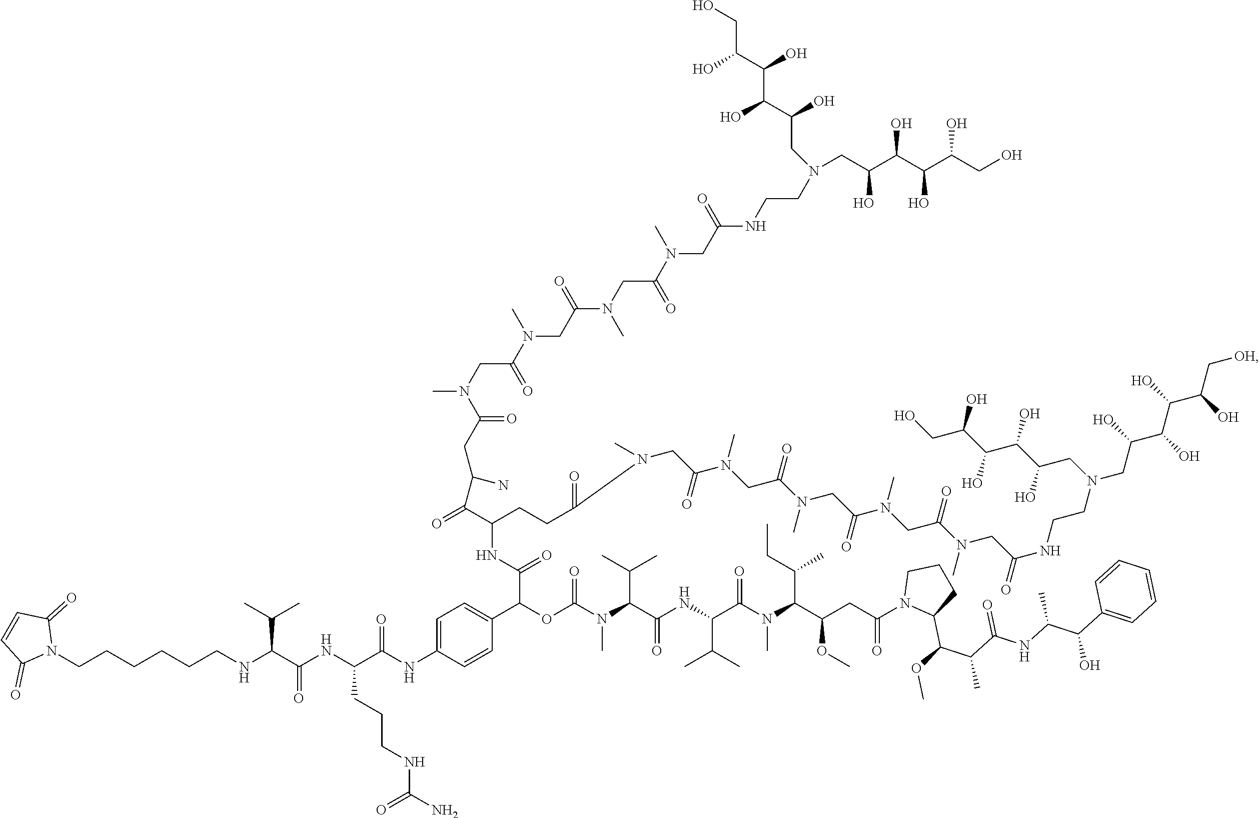 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NC(CCC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)C(=O)C(N)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NCCCCCCN2C(=O)C=CC2=O)C(C)C)cc1)C(C)C]

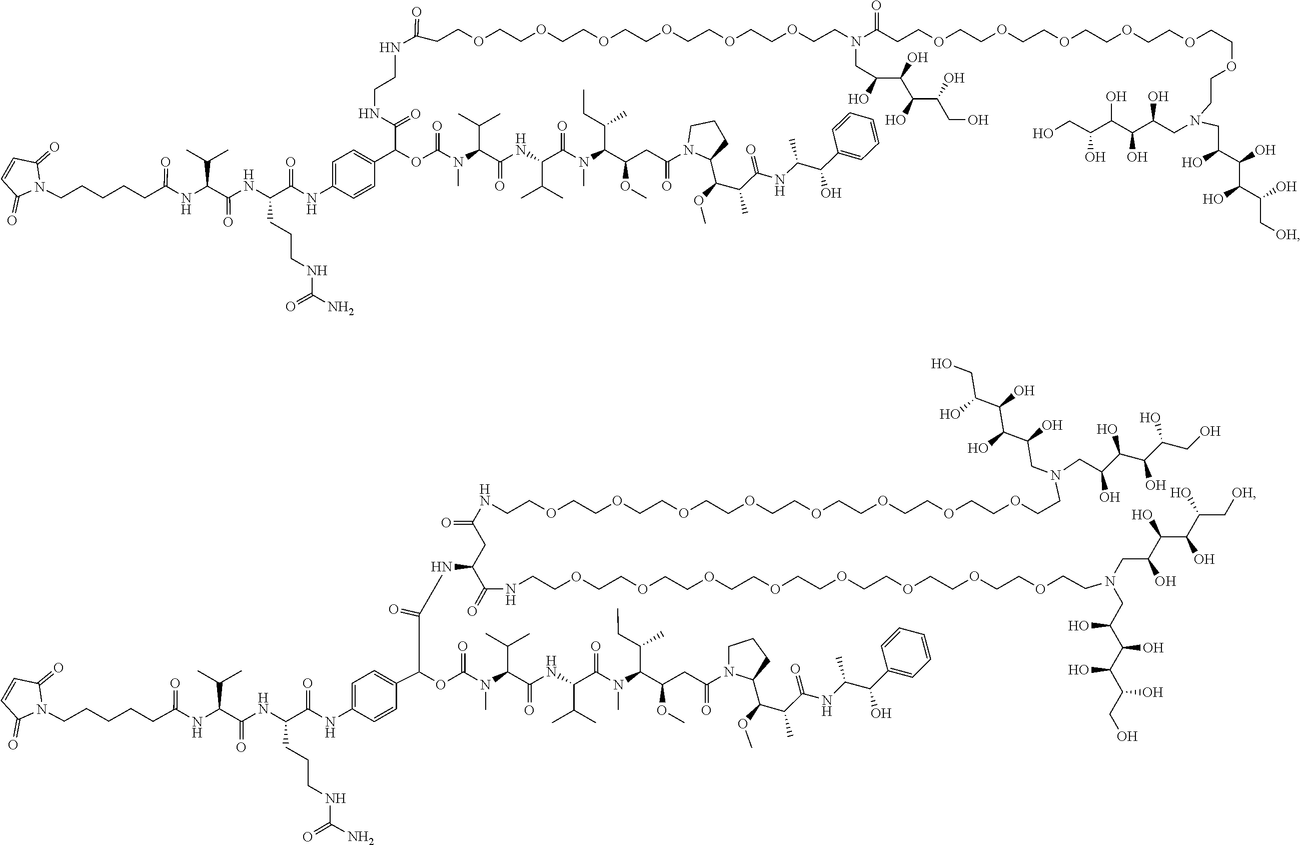

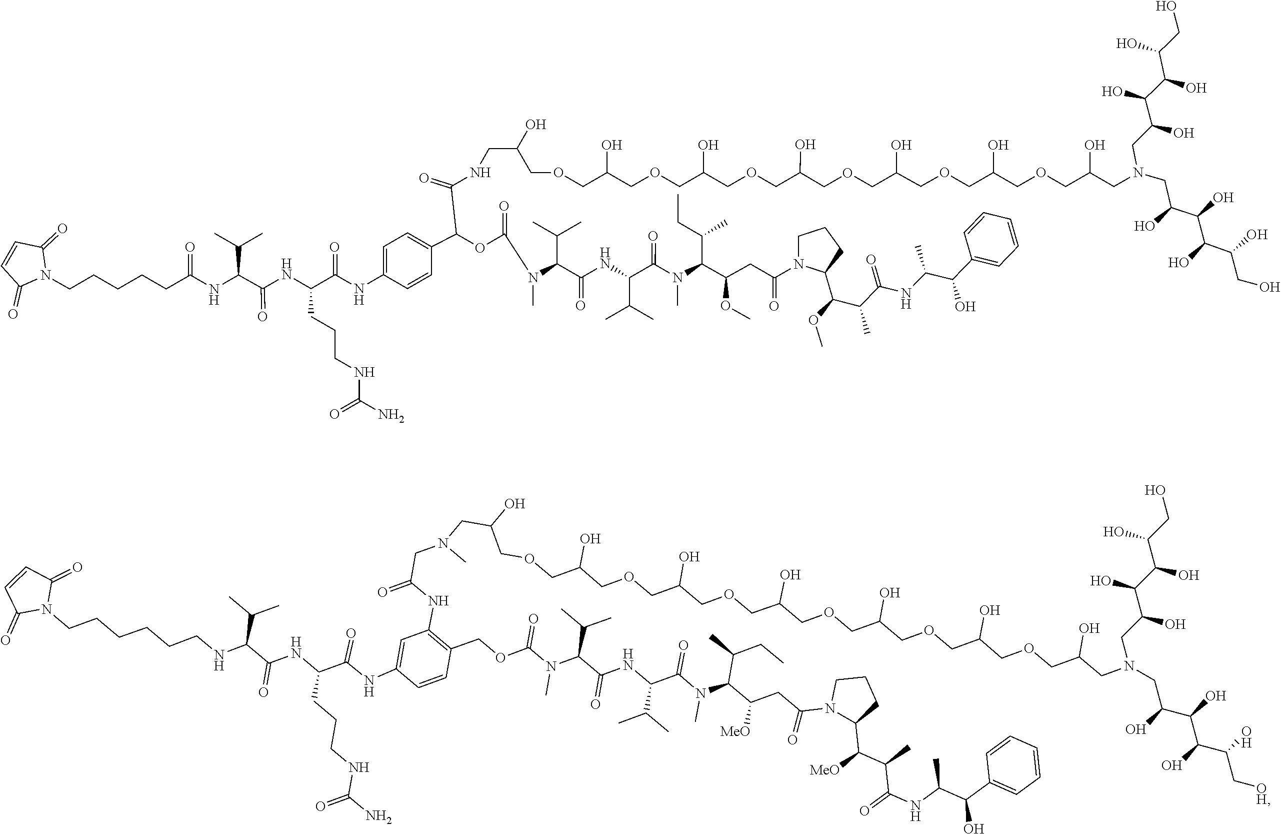

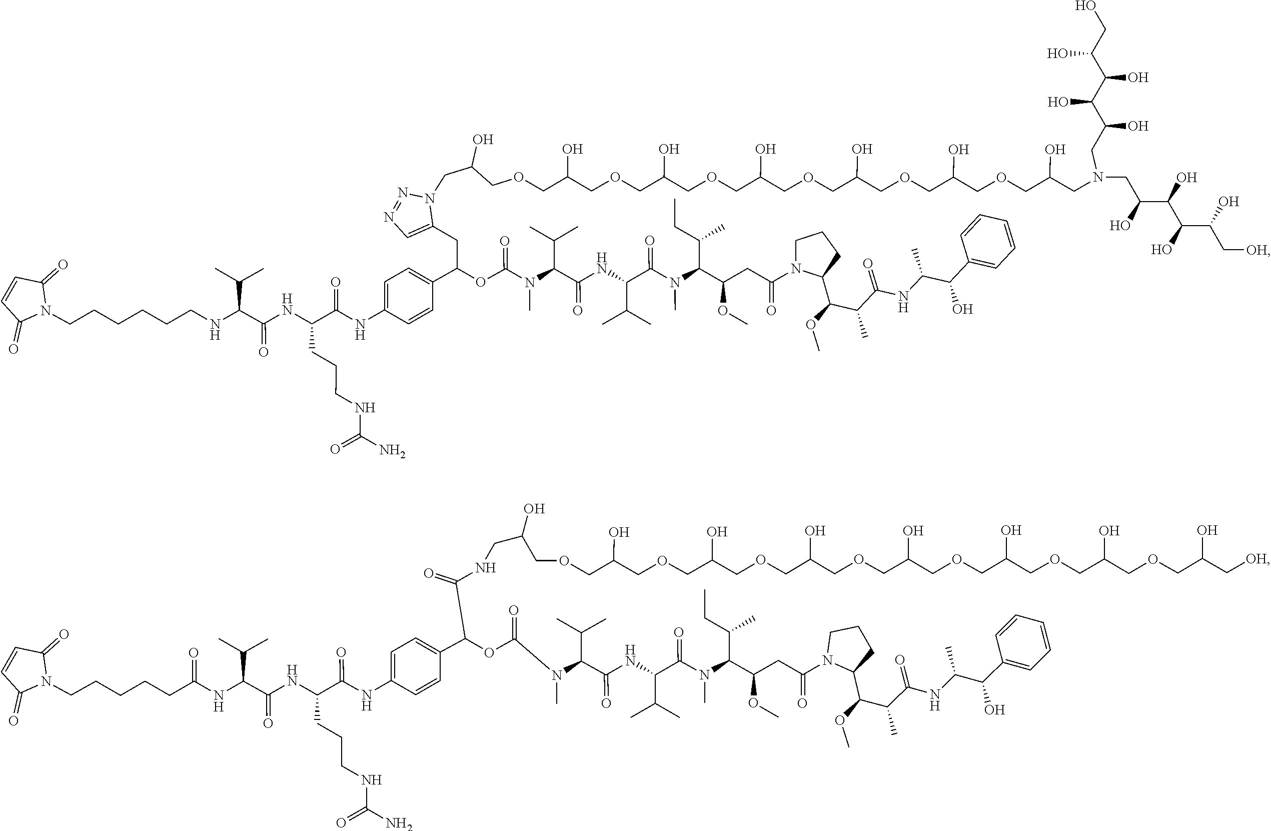

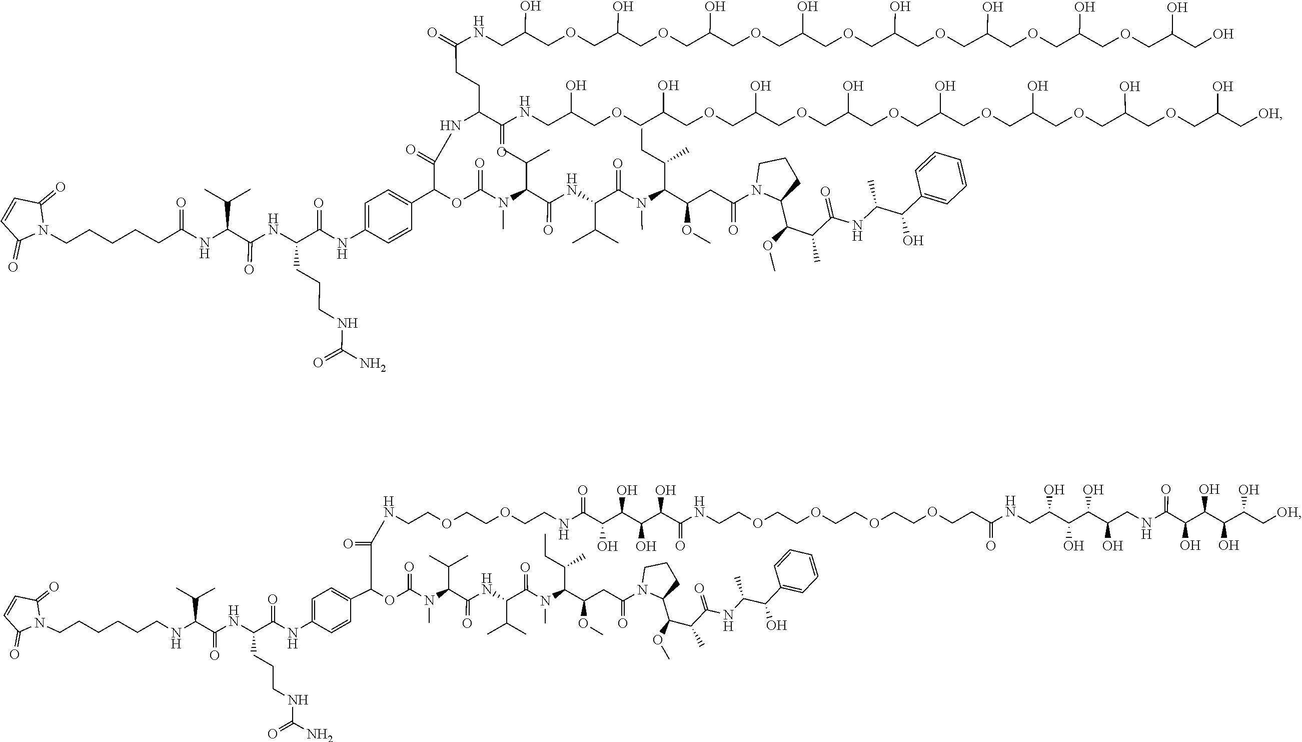

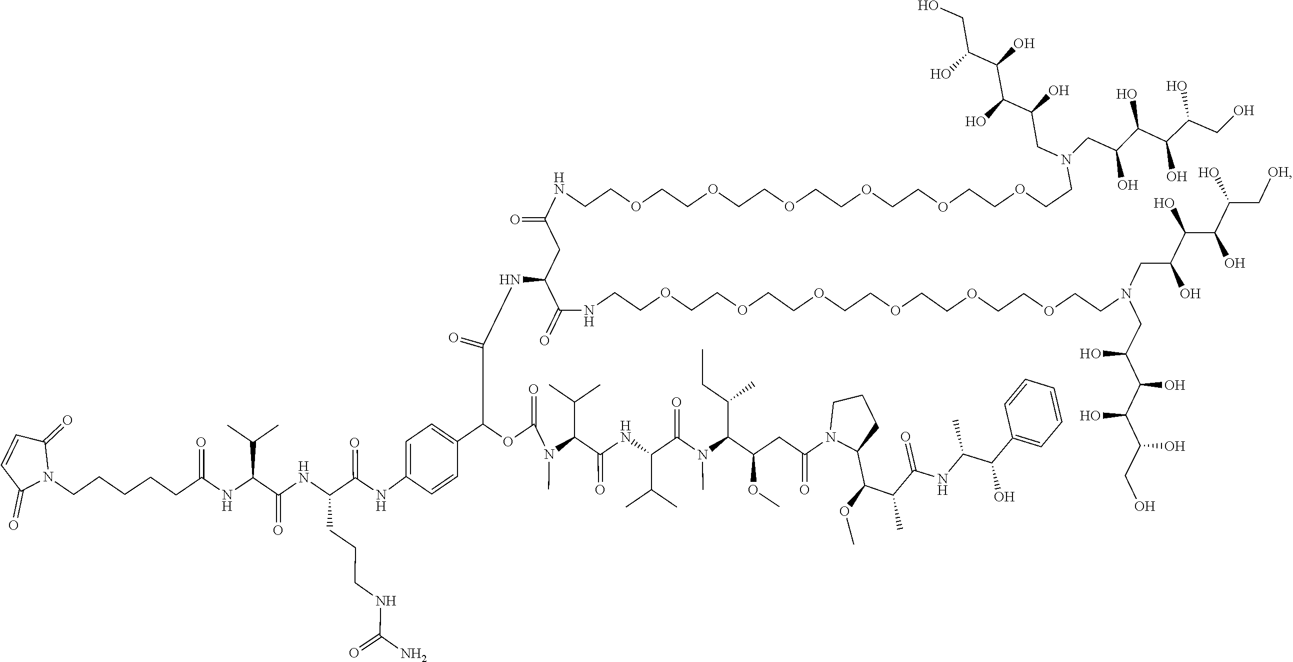 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

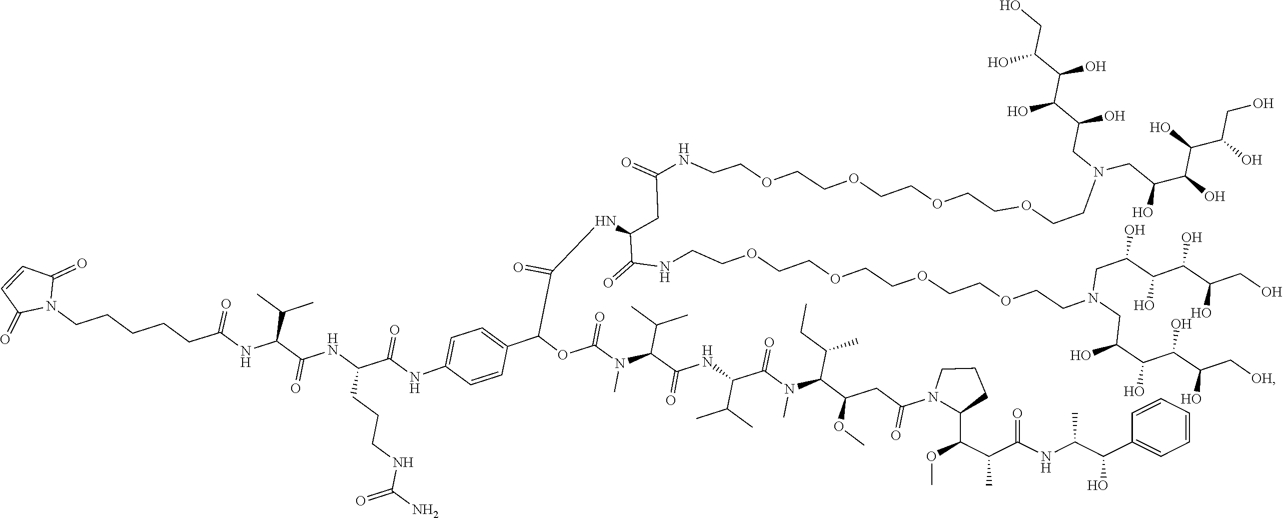 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCN(C[C@H](O)[C@H](O)[C@@H](O)[C@@H](O)CO)C[C@H](O)[C@@H](O)[C@H](O)[C@H](O)CO)C(=O)NCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

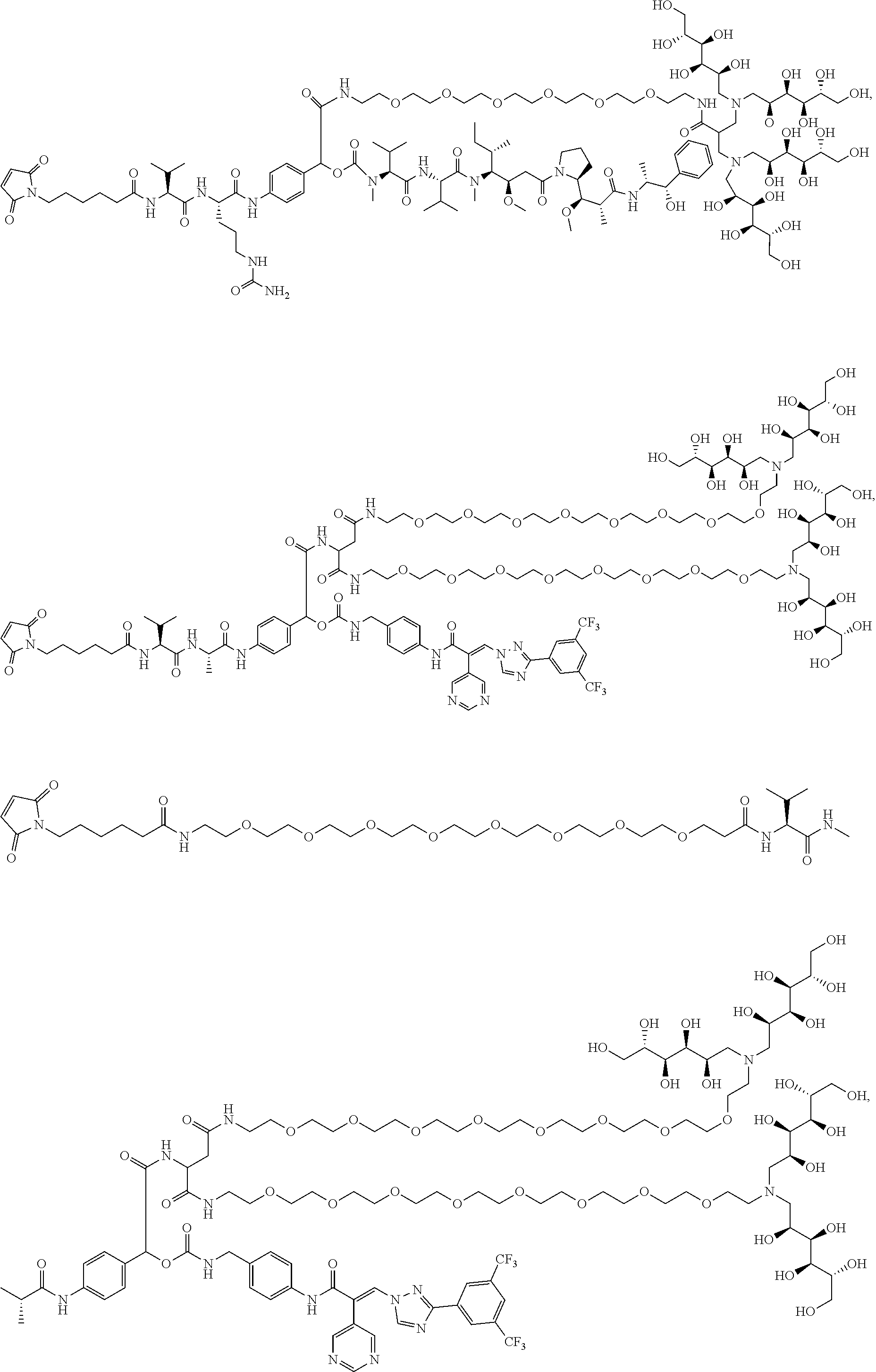

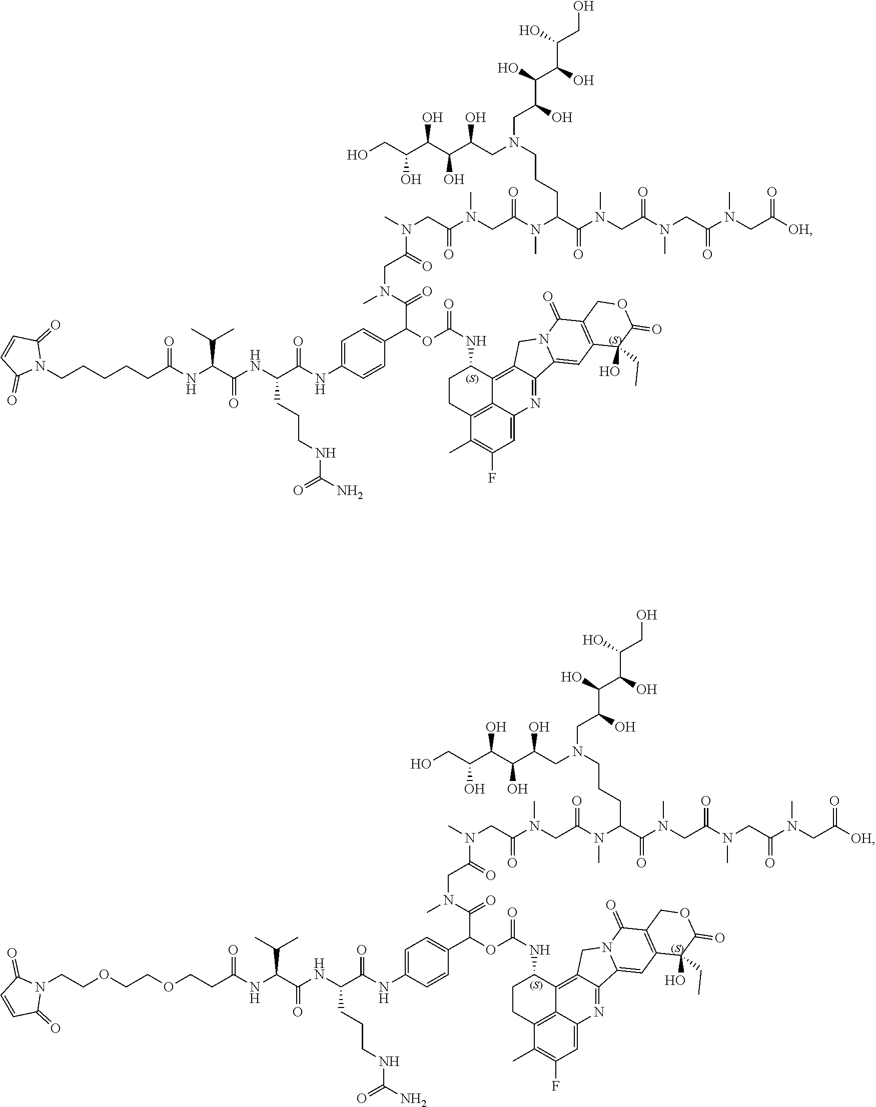

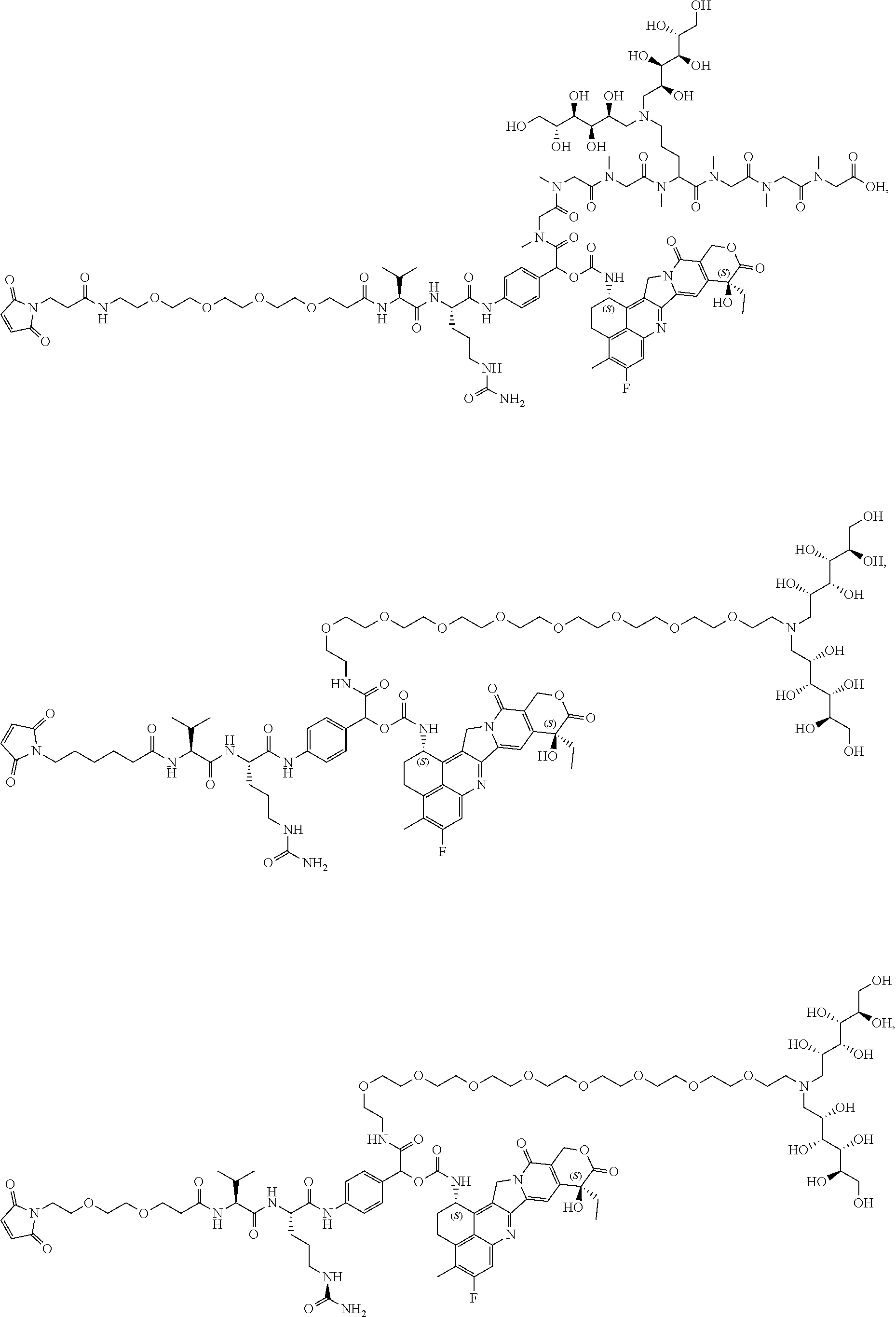

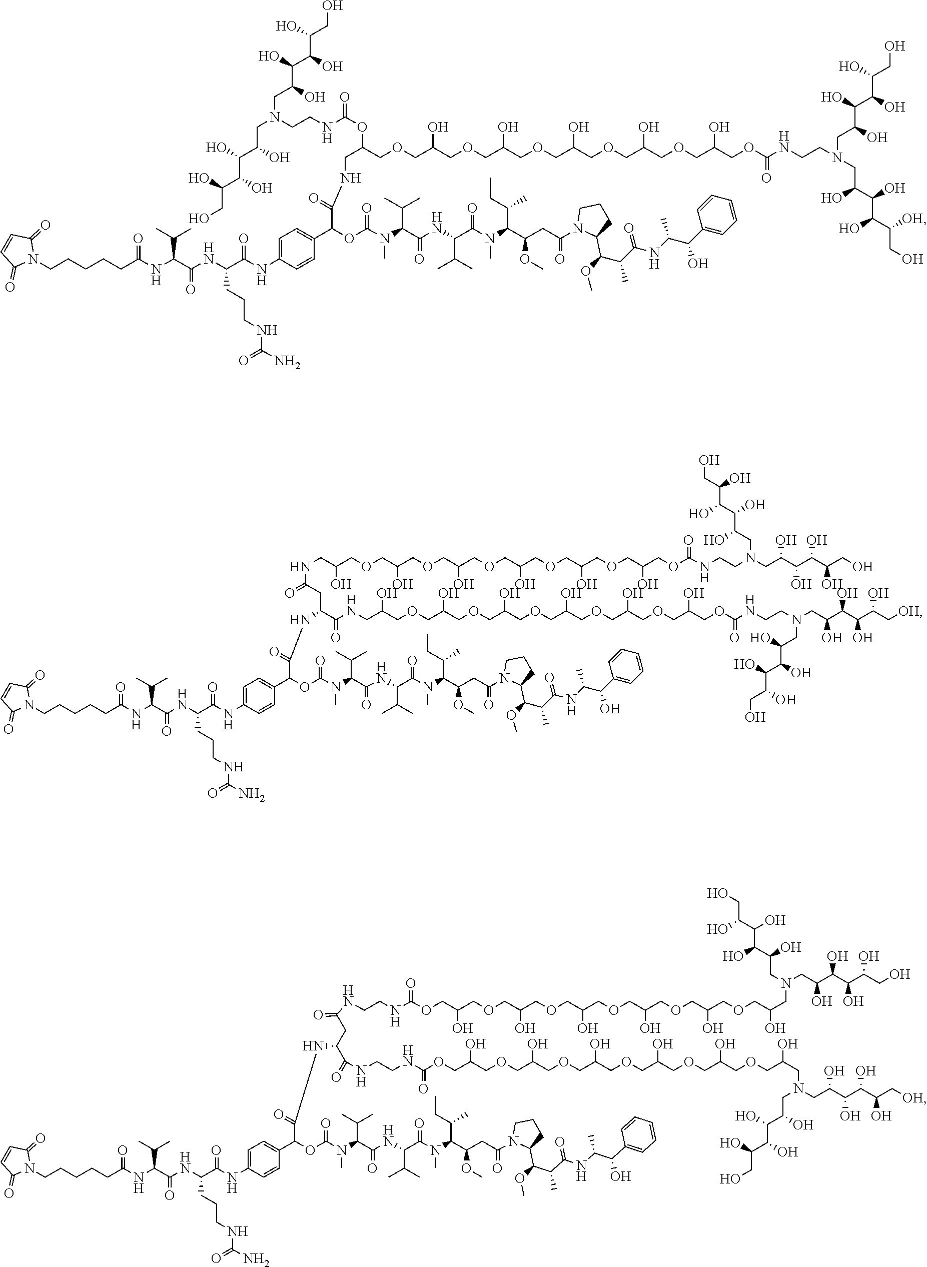

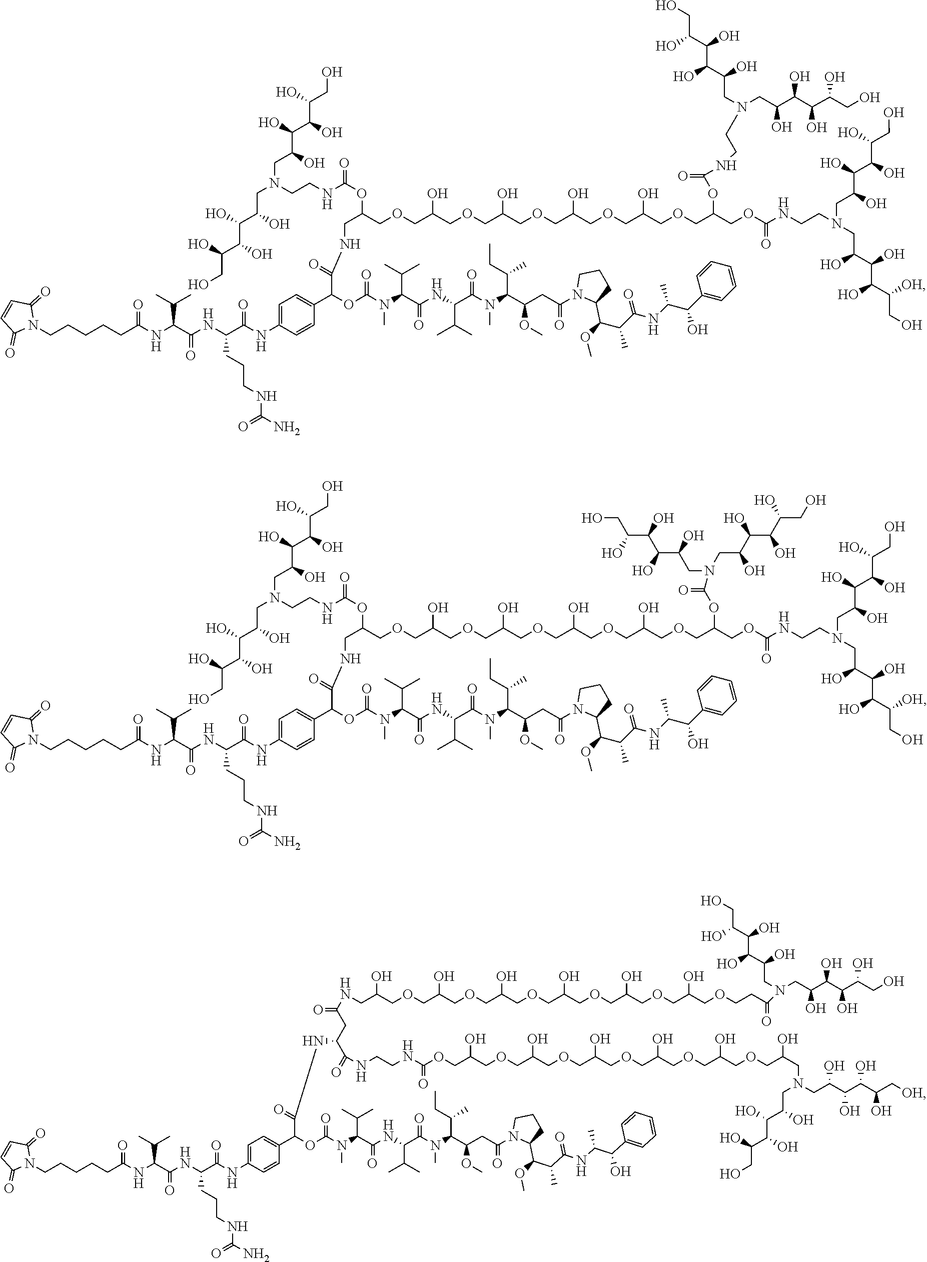

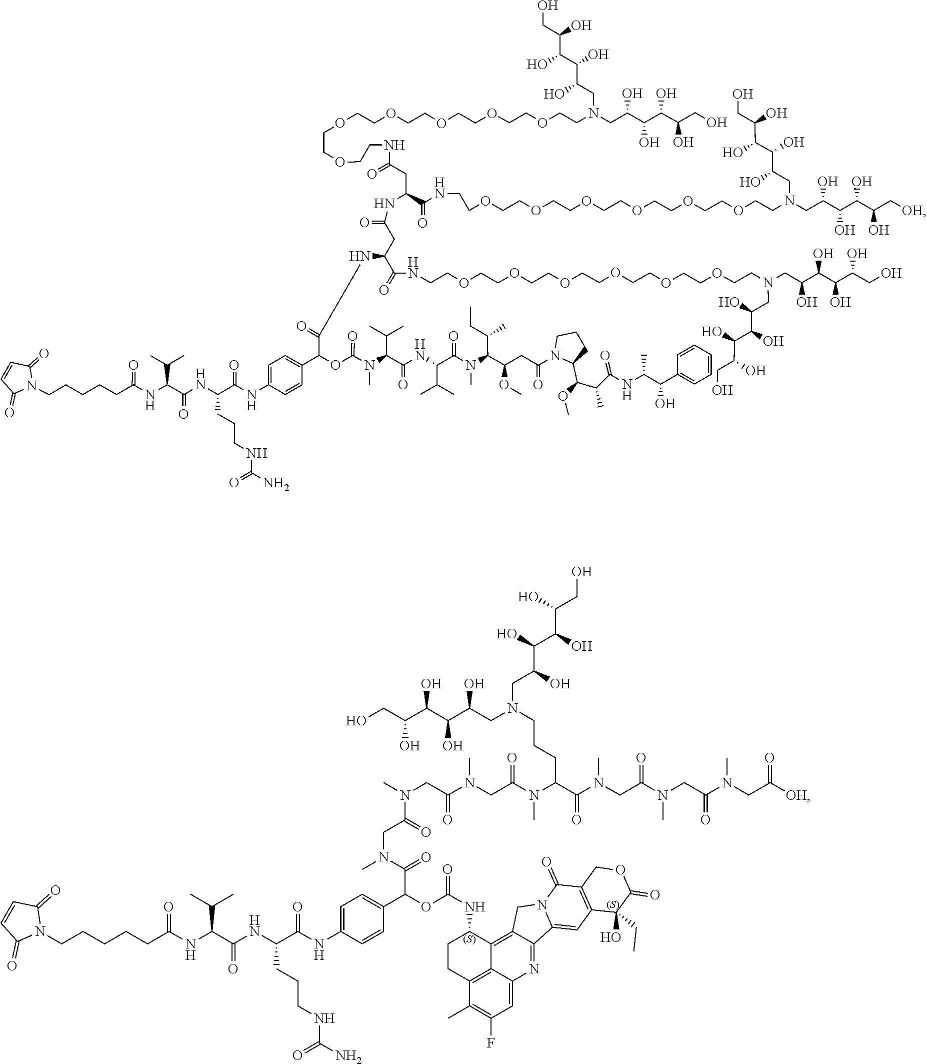

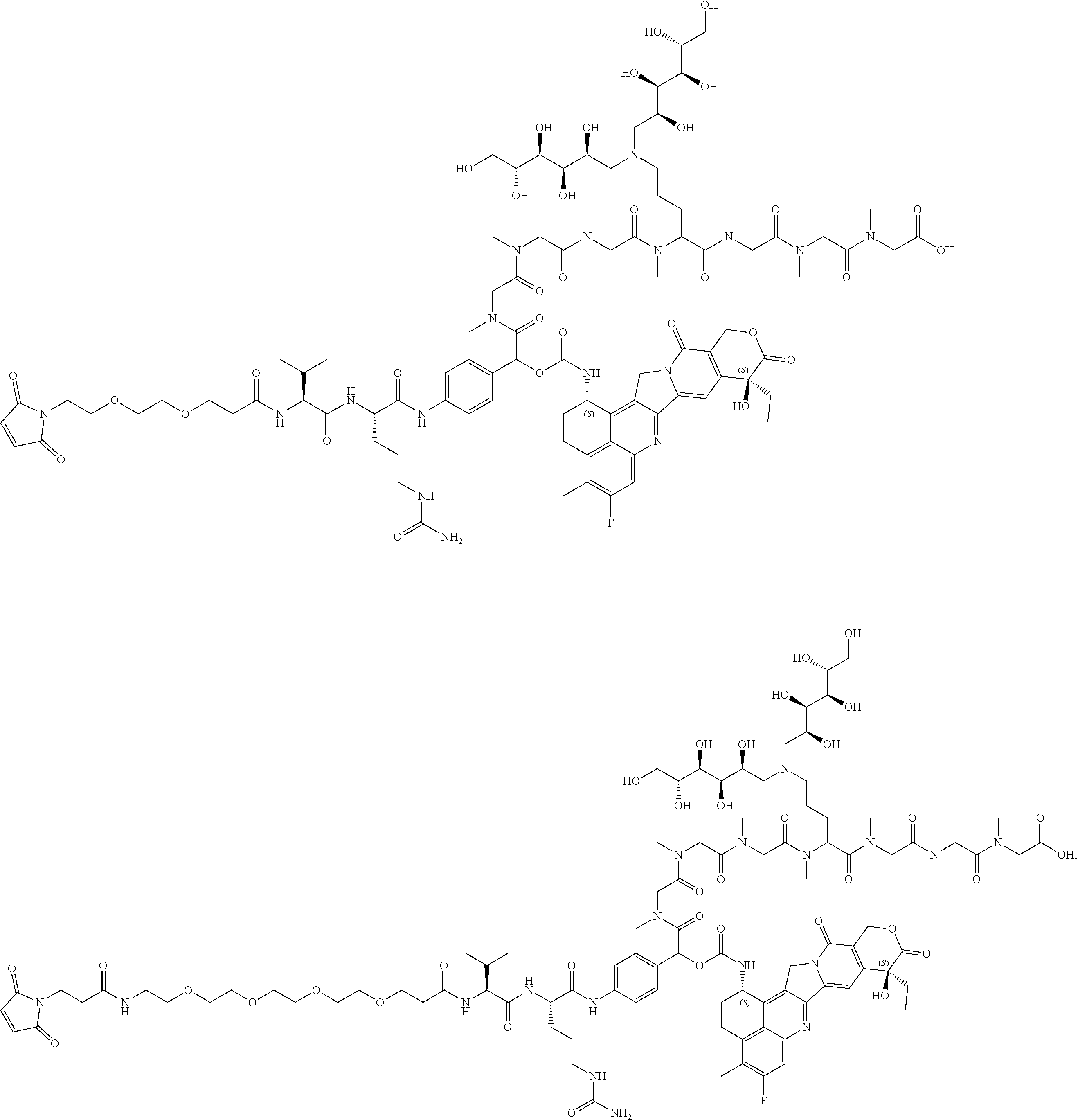

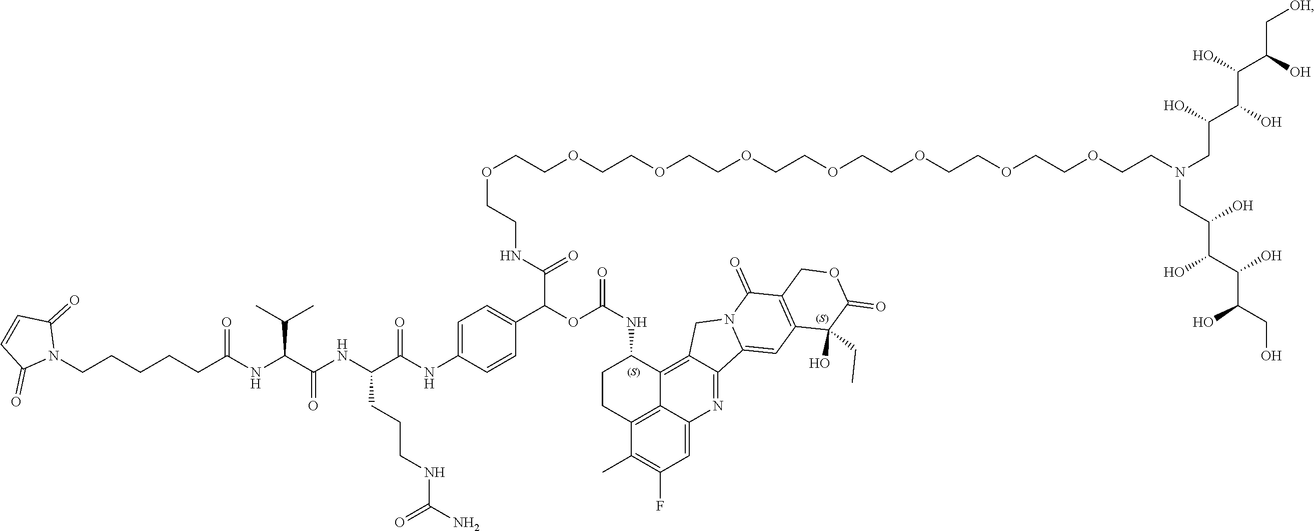

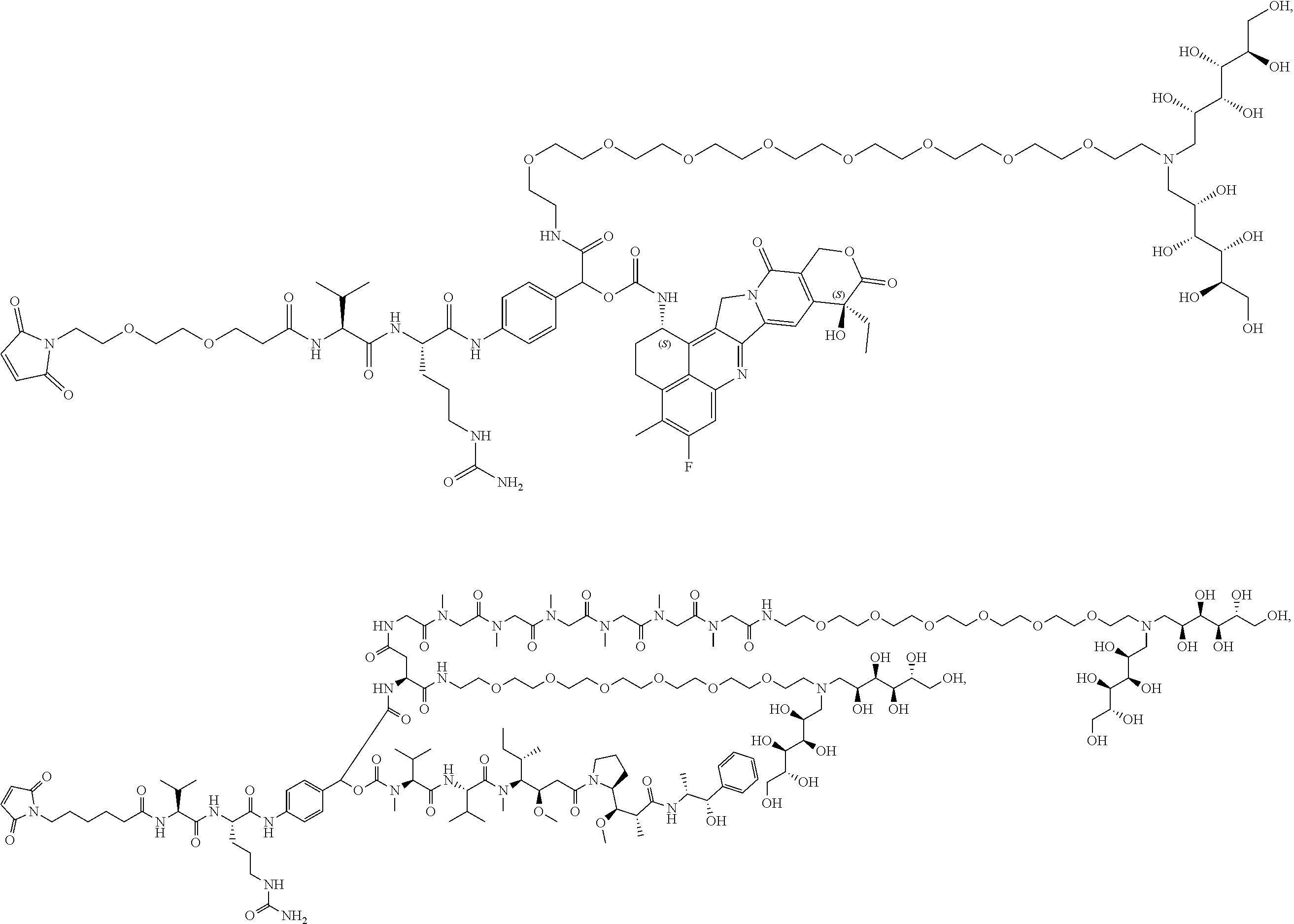

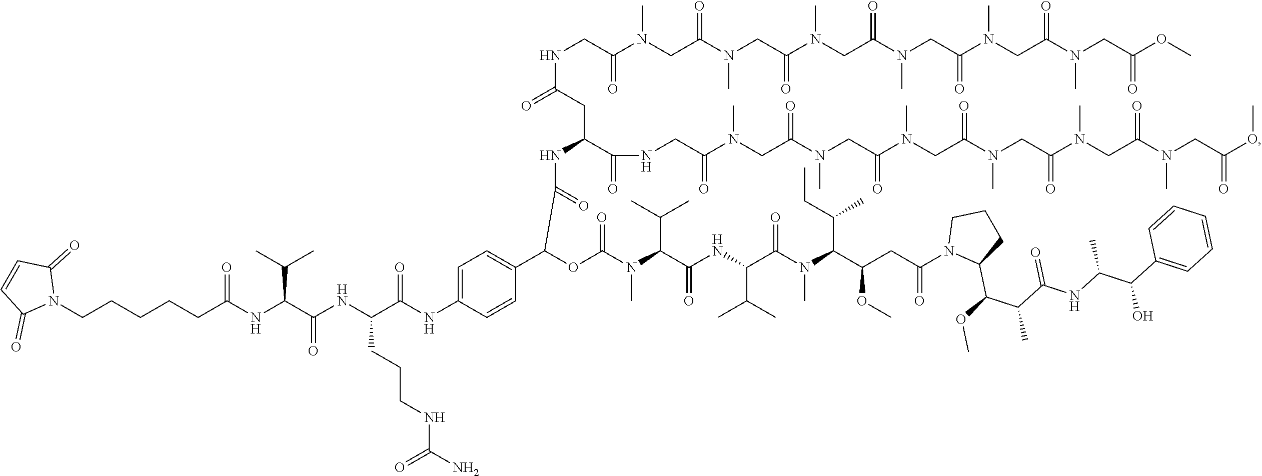 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCC(=O)N(C)CC(=O)N(C)CC(=O)N(C)CC(=O)N(C)CC(=O)N(C)CC(=O)N(C)CC(=O)OC)C(=O)NCC(=O)N(C)CC(=O)N(C)CC(=O)N(C)CC(=O)N(C)CC(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

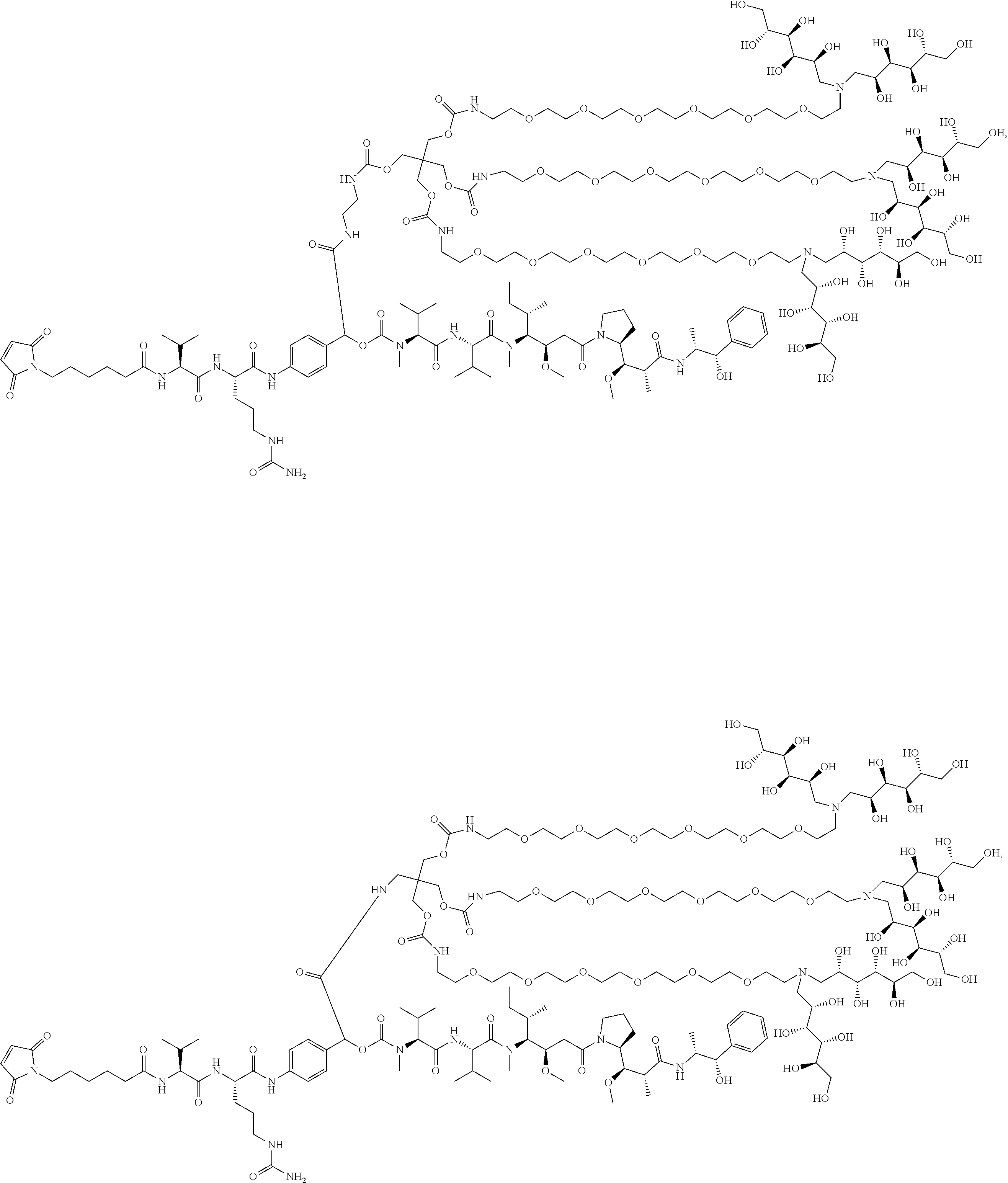

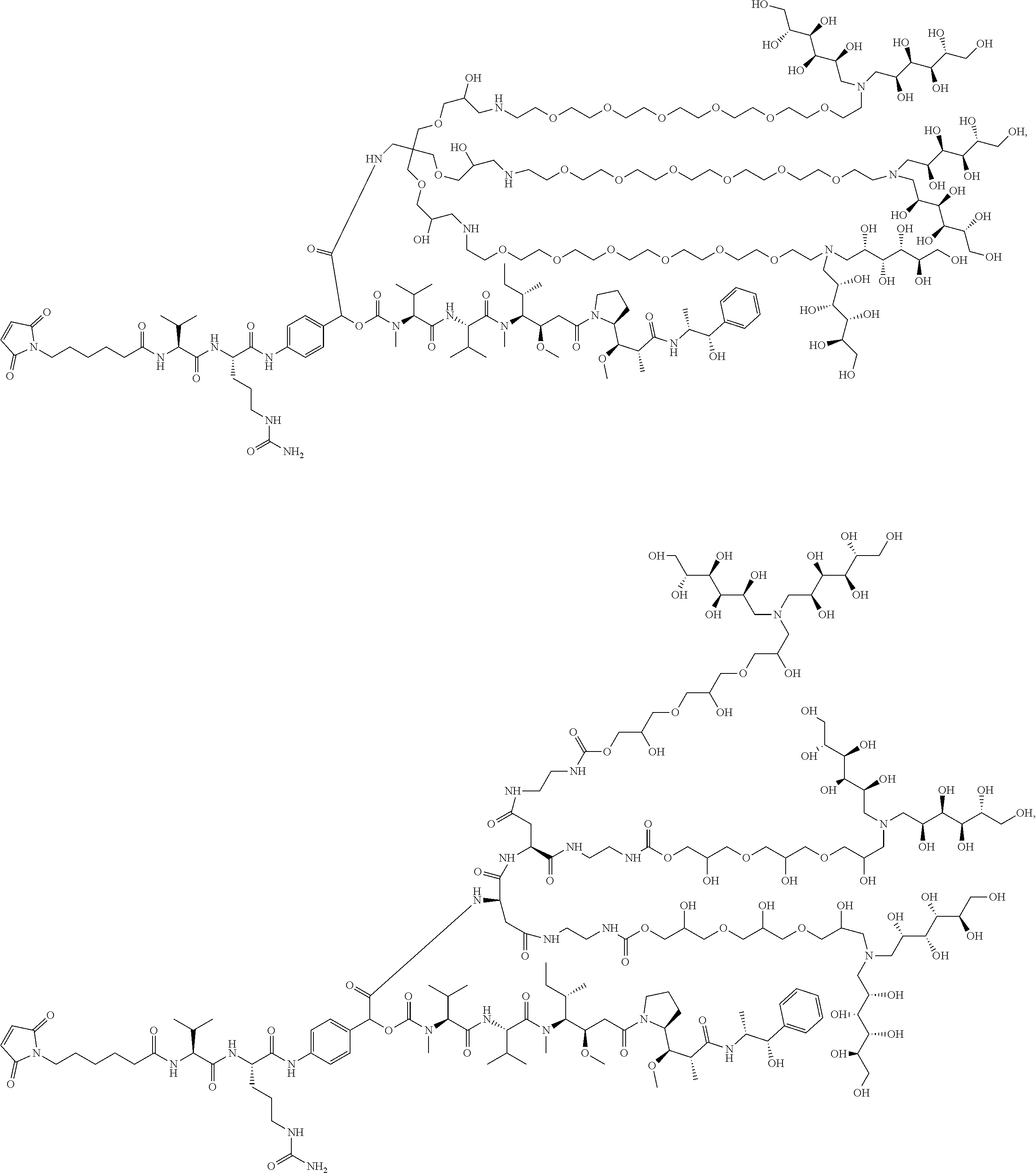

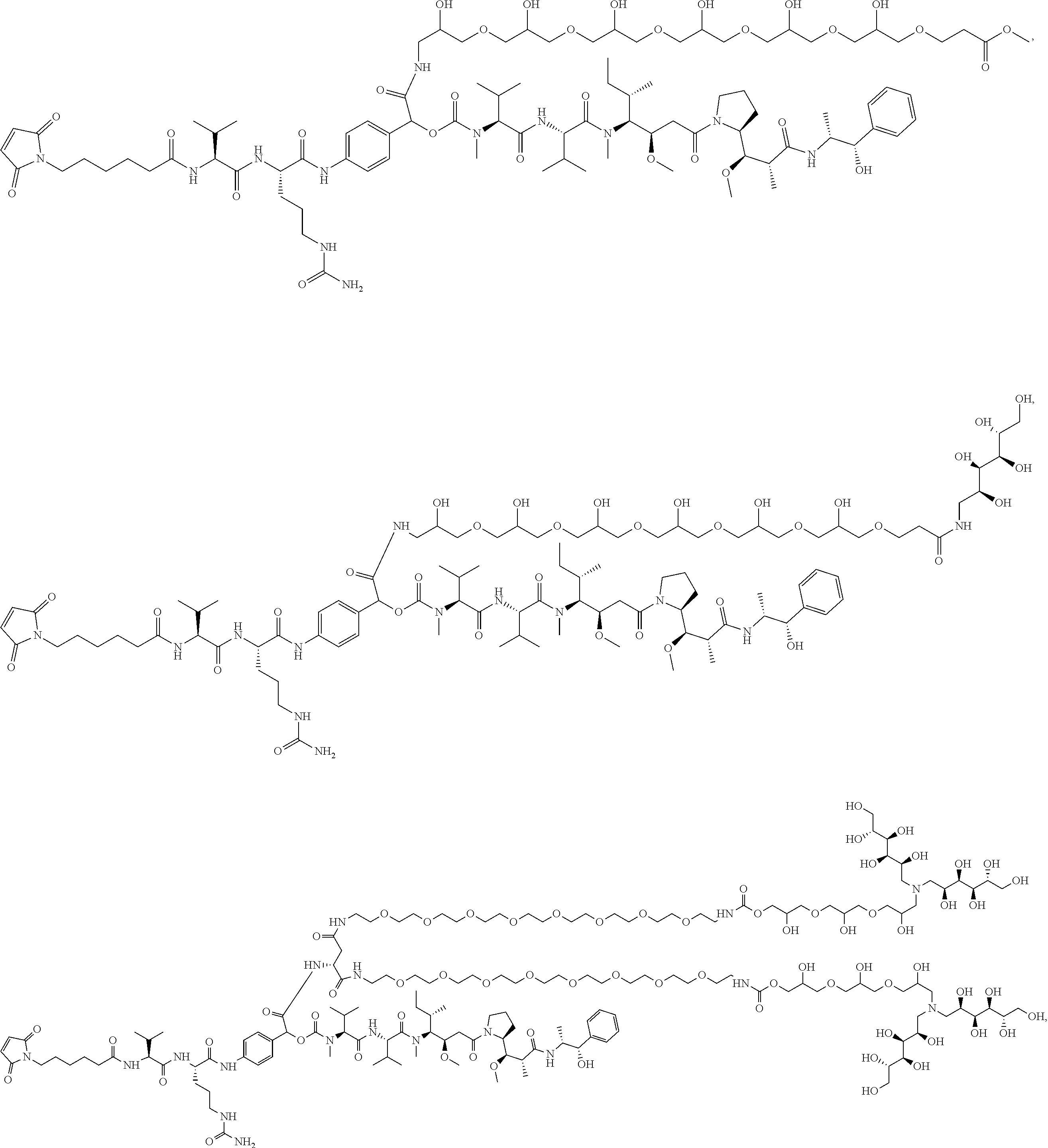

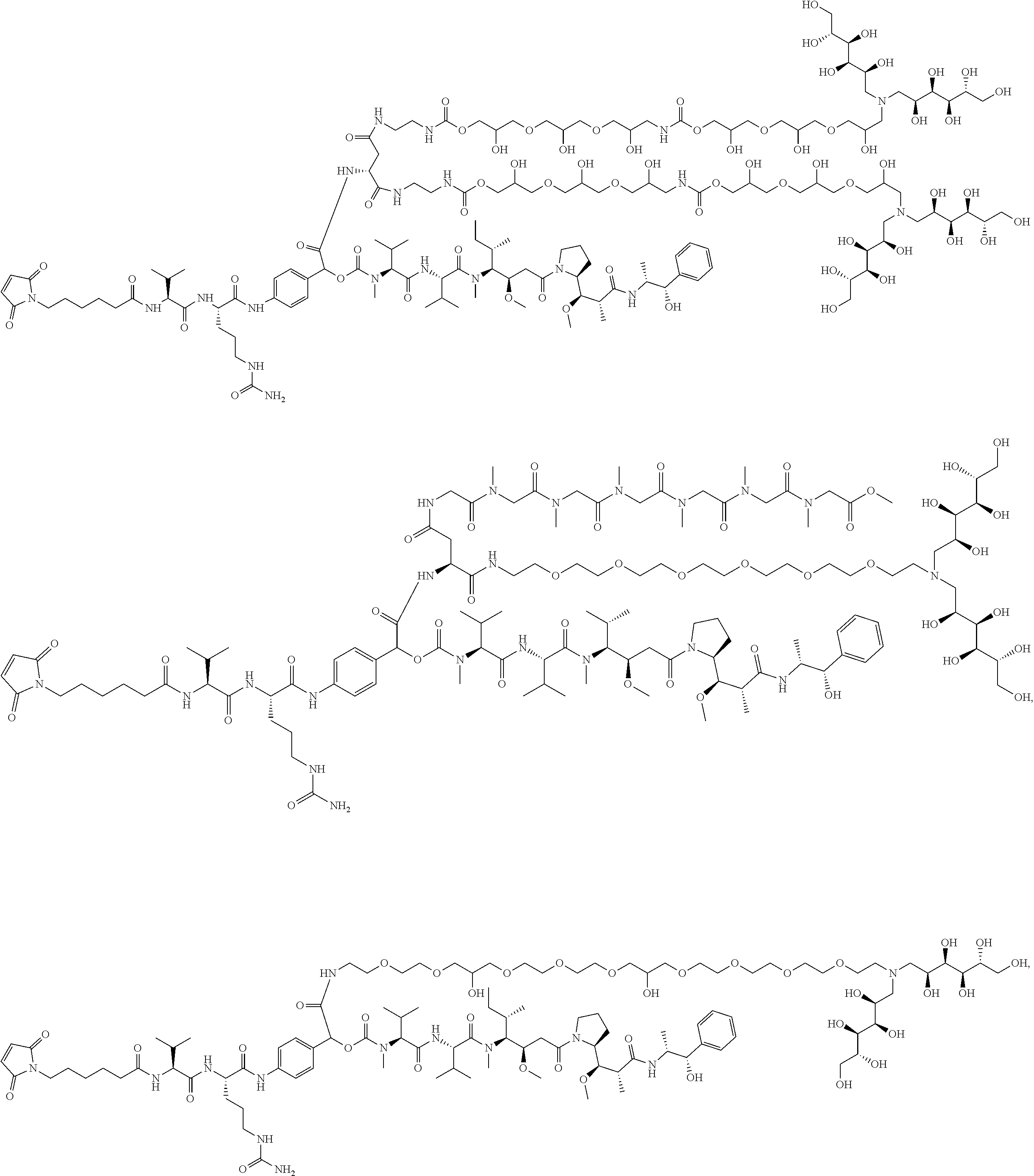

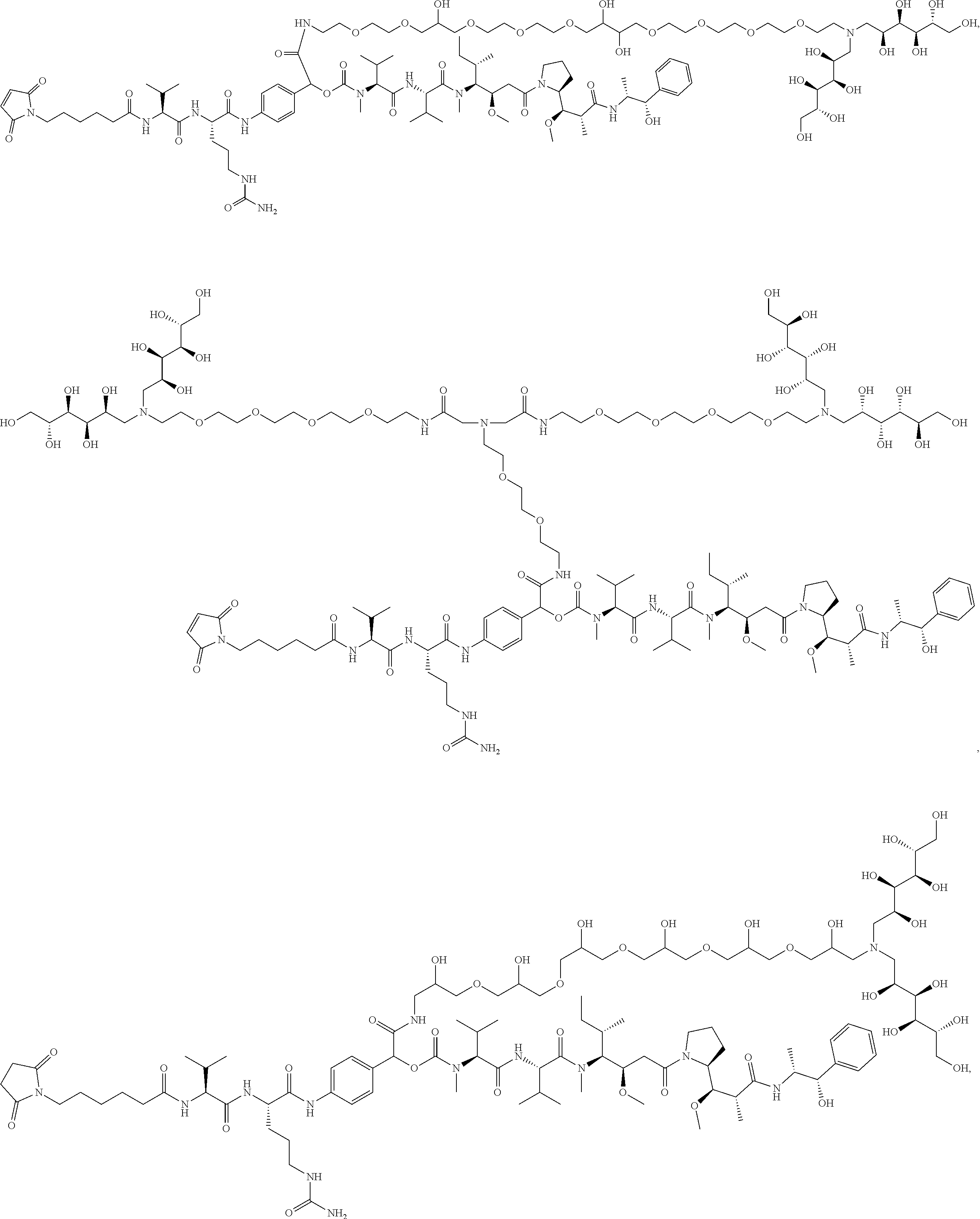

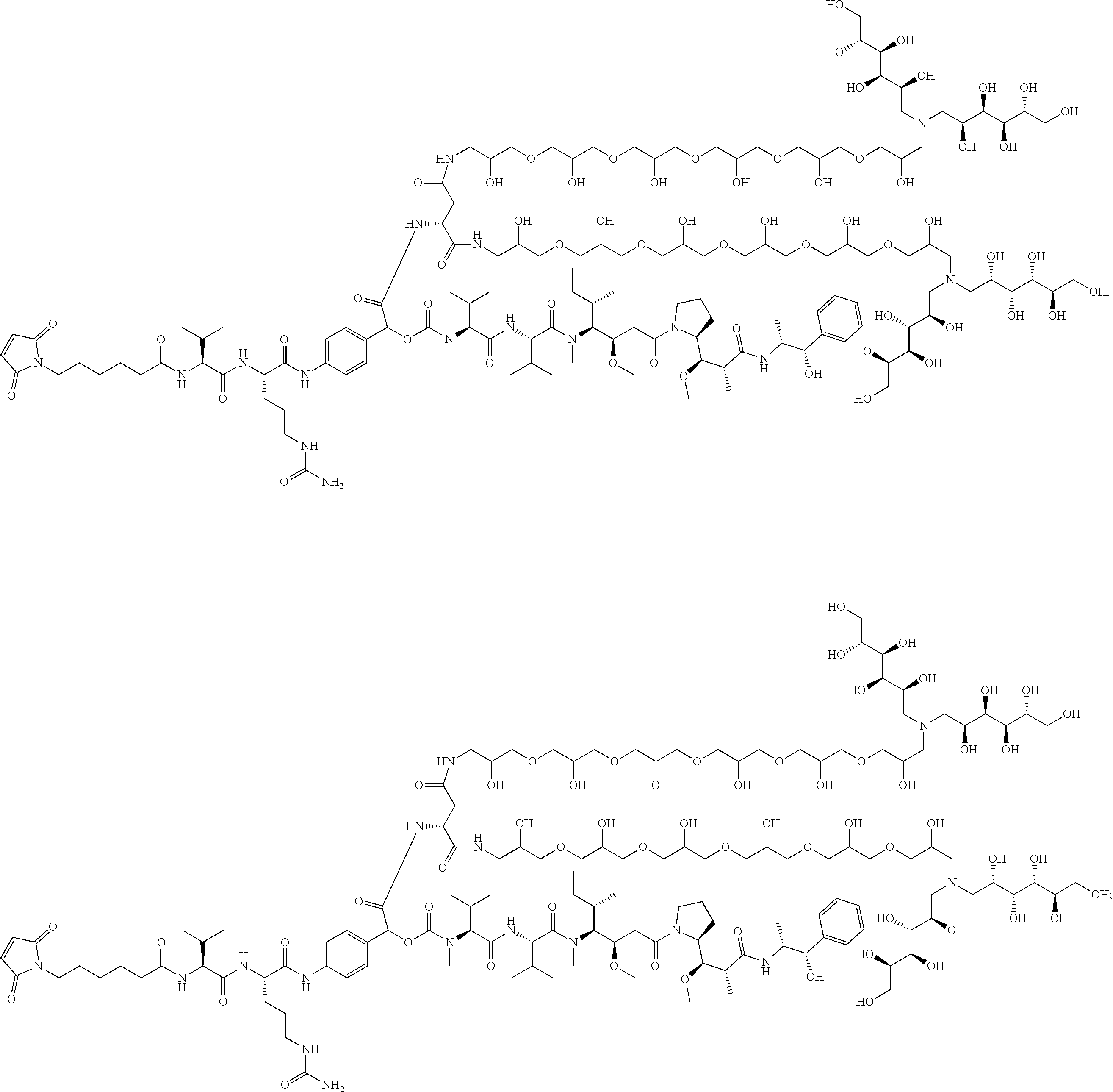

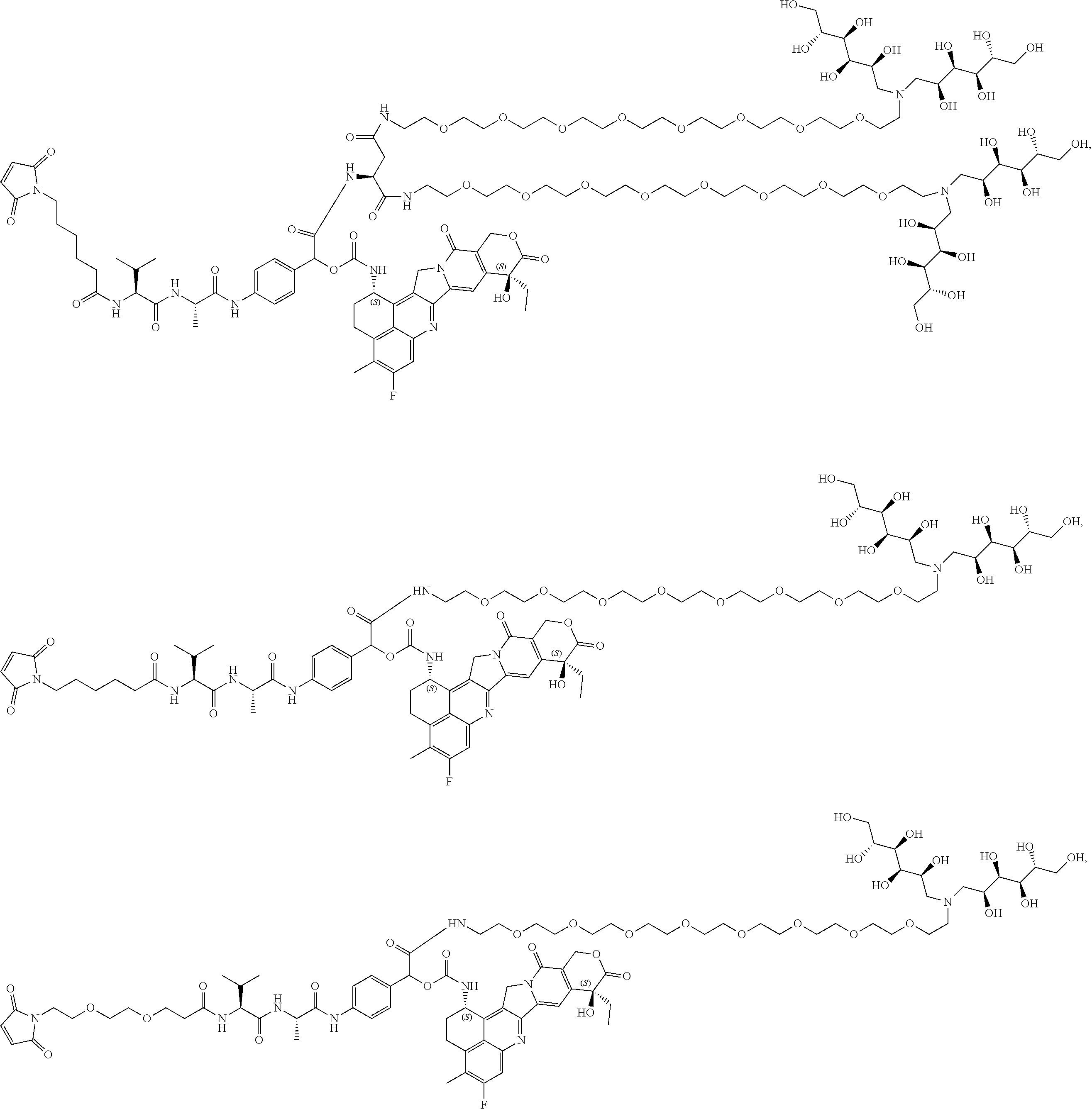

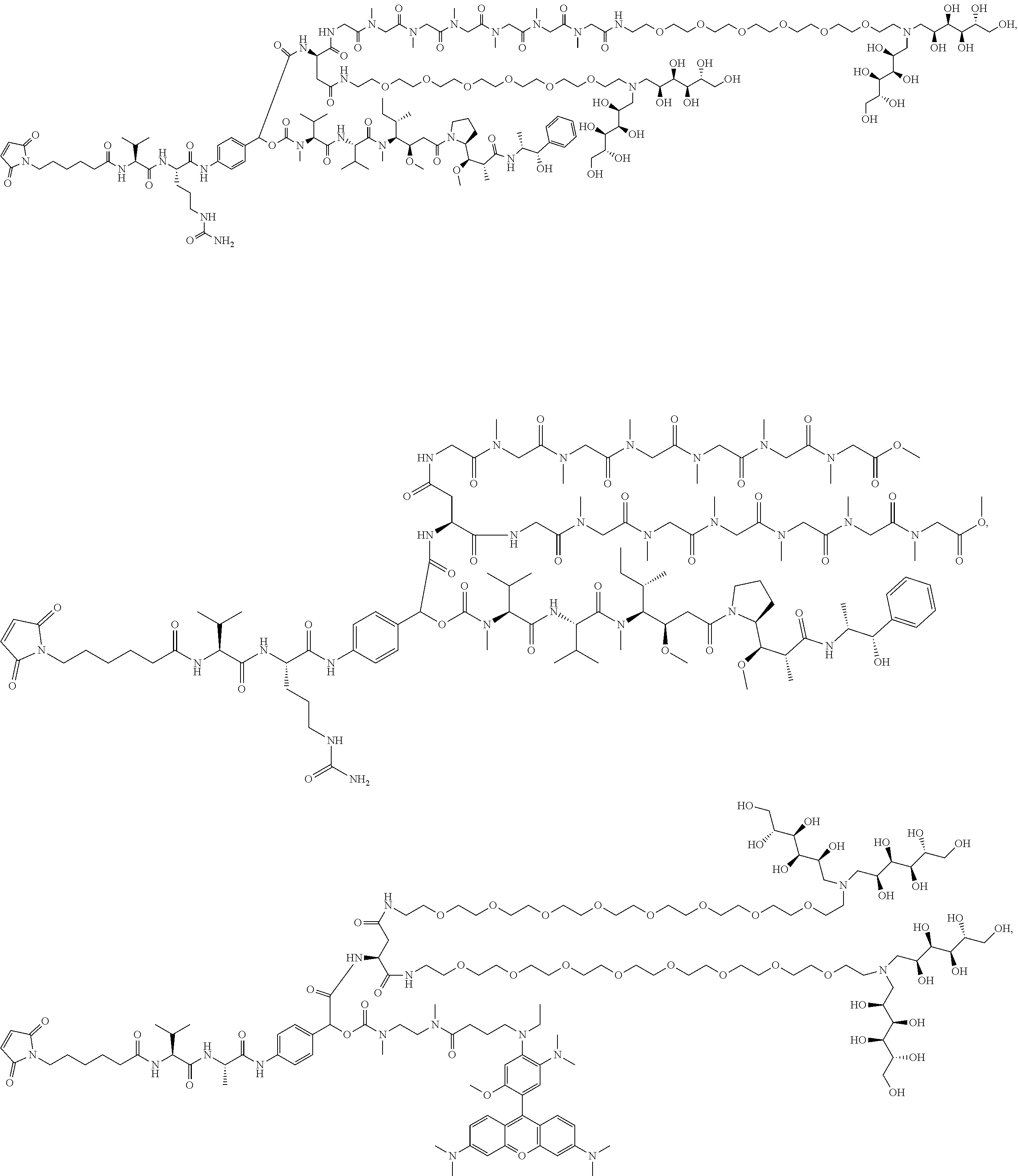

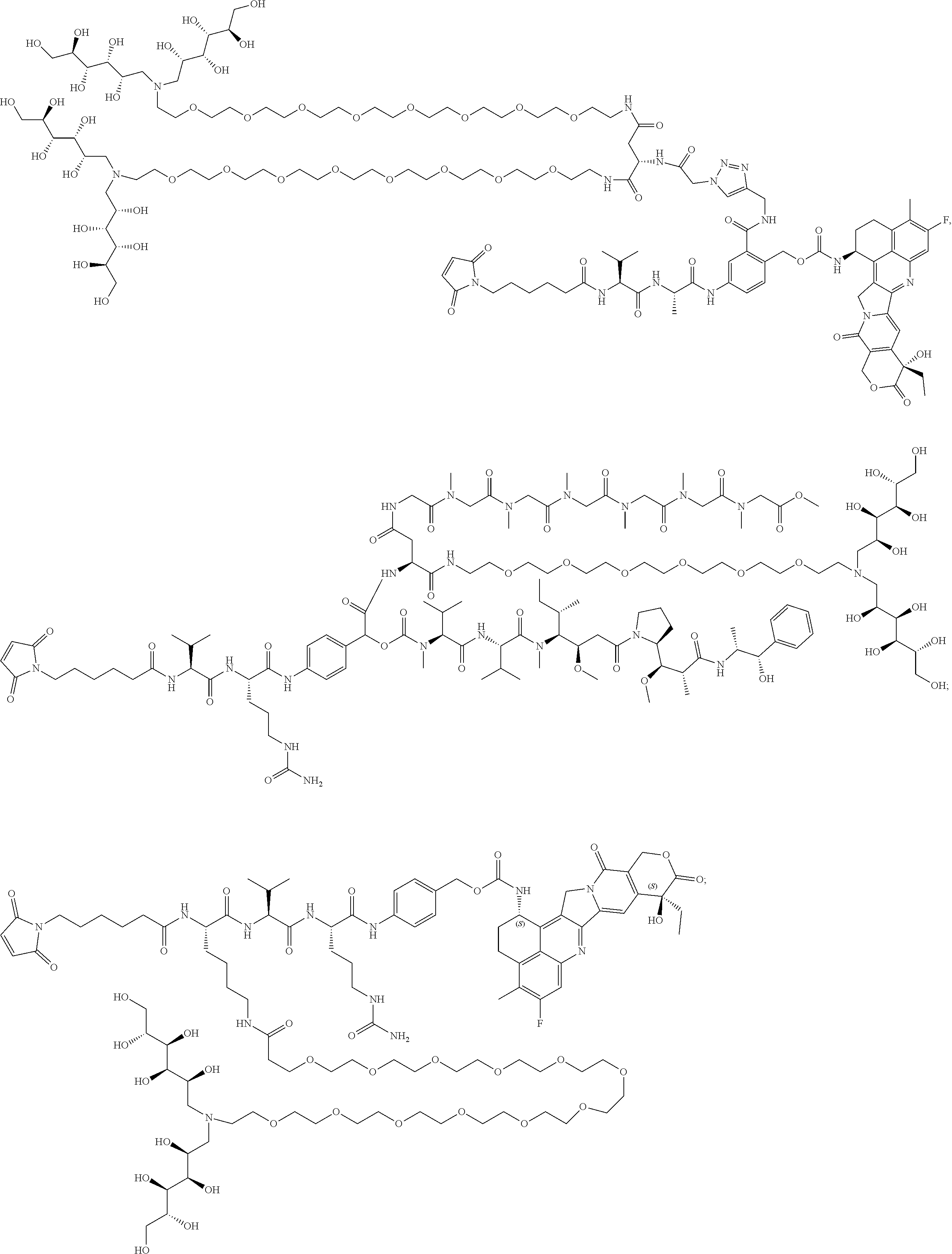

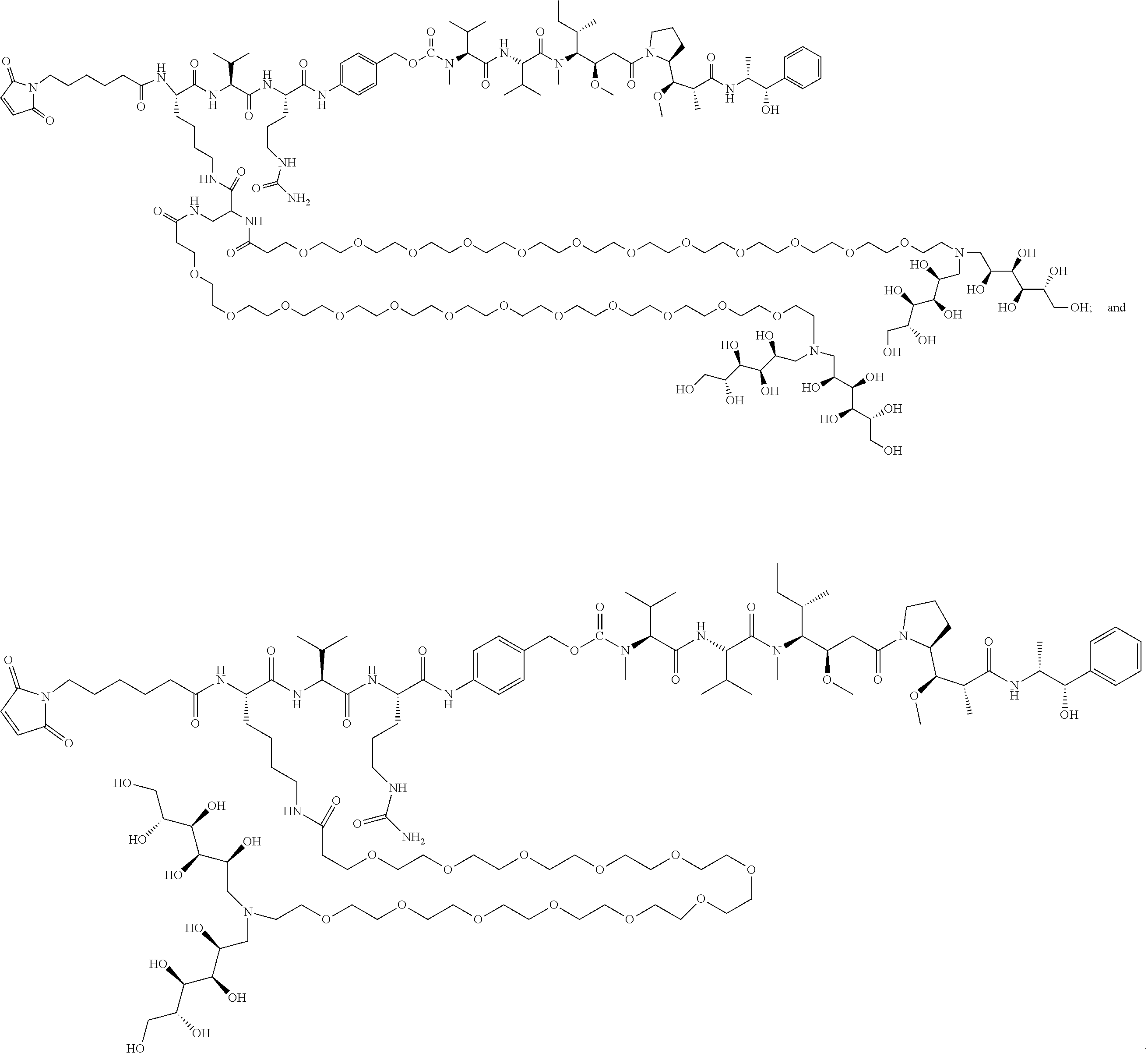

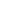

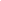

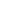

Embodiment 136. The drug-linker compound of Embodiment 109, the drug-linker compound has one of the following structure:

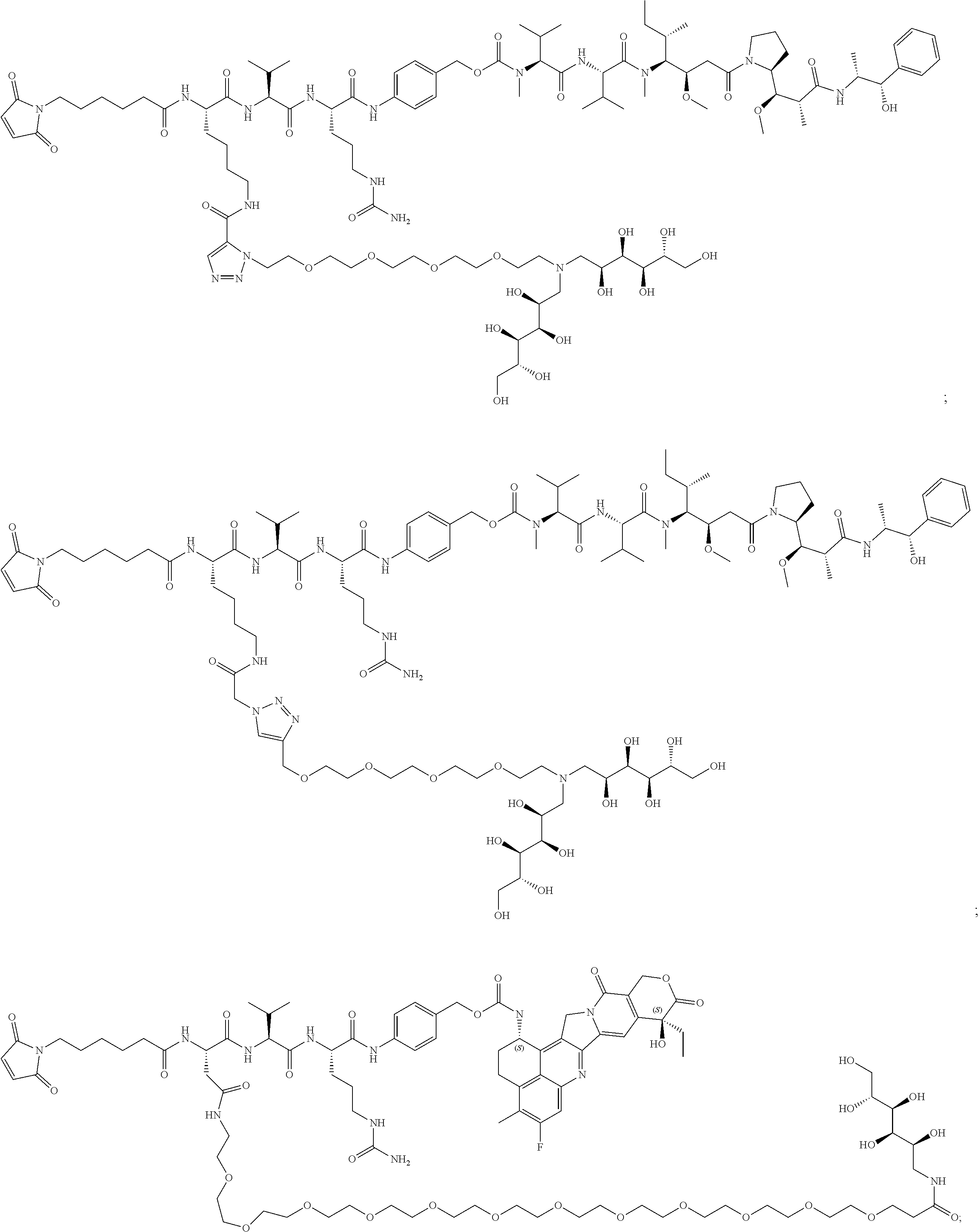

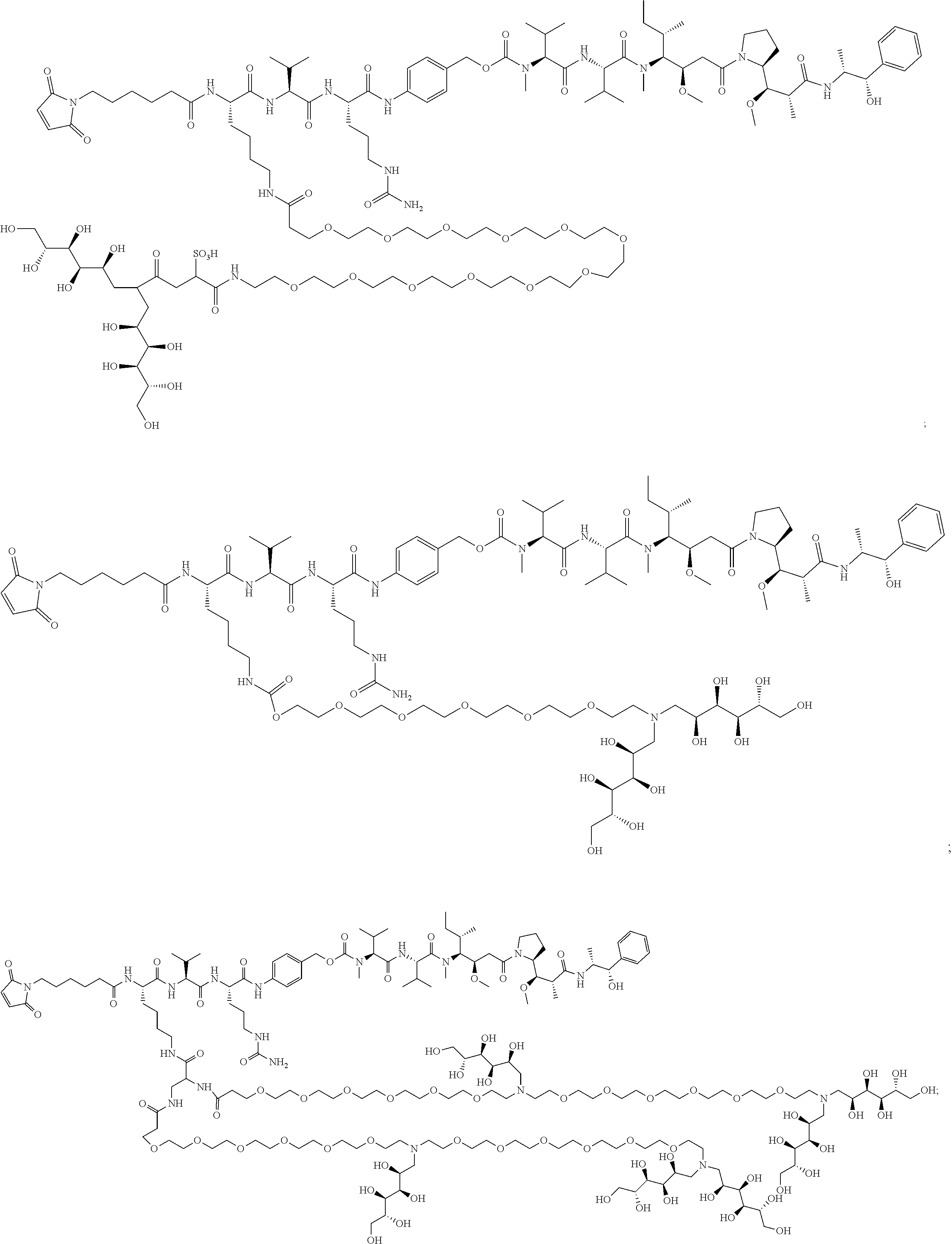

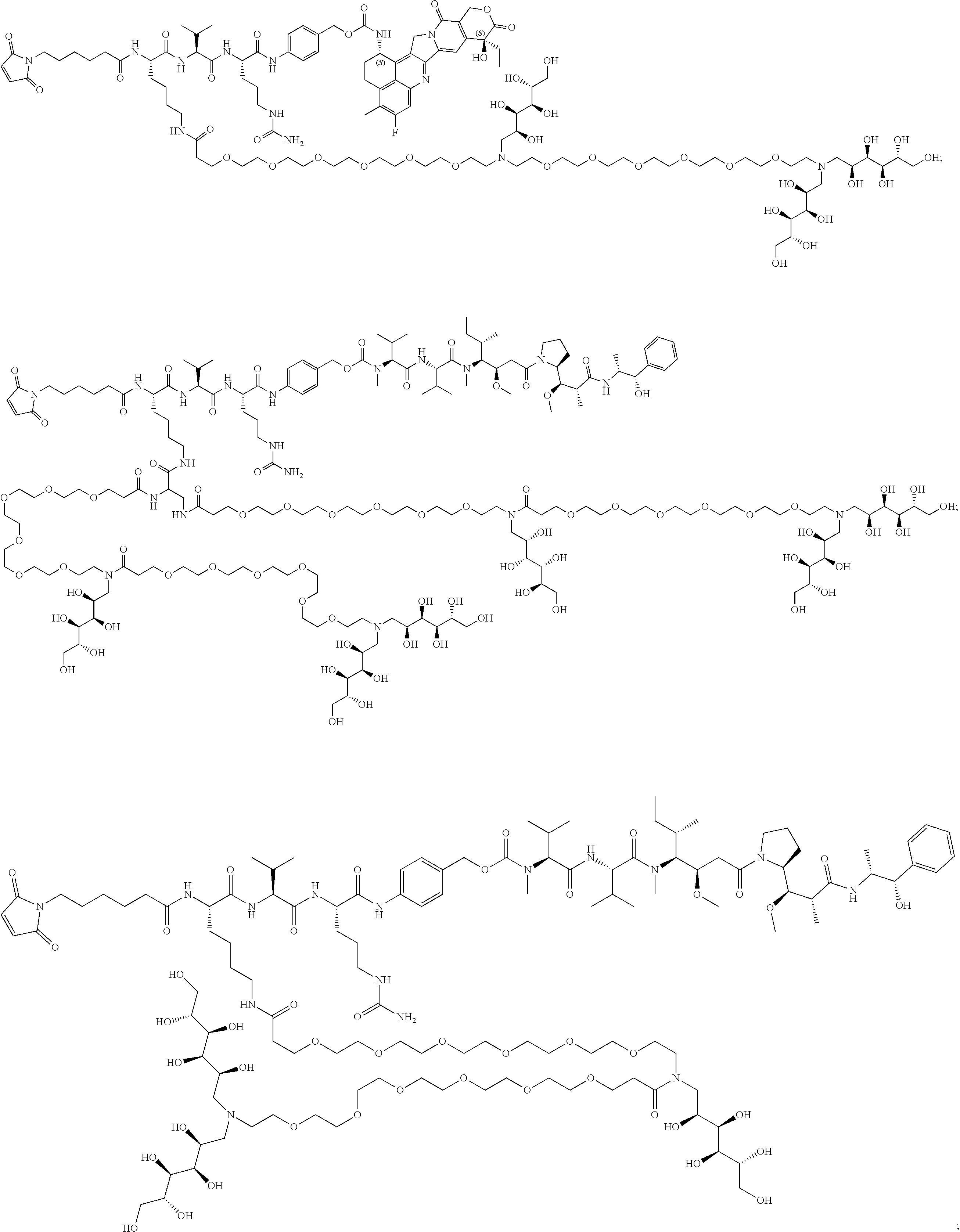

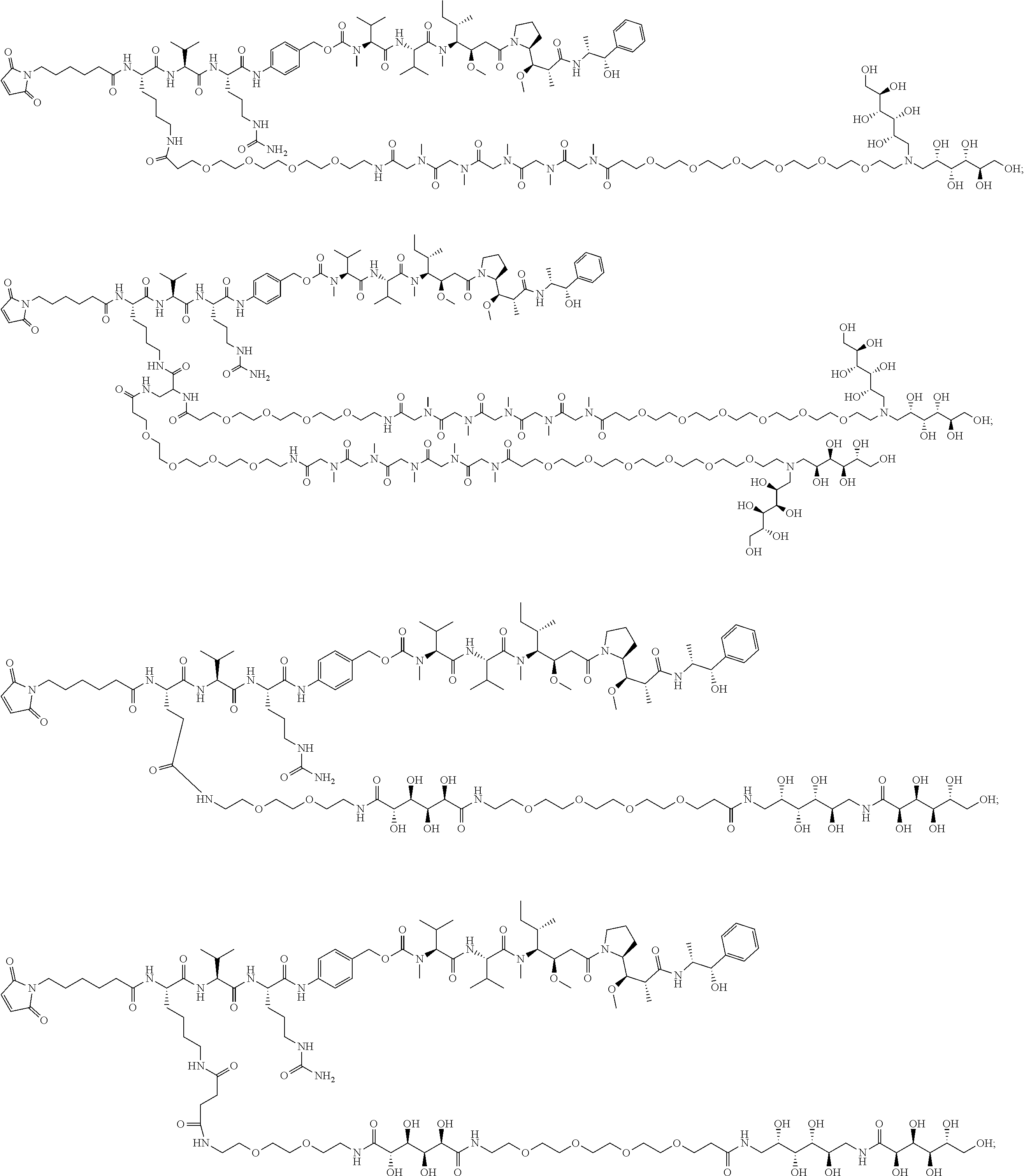

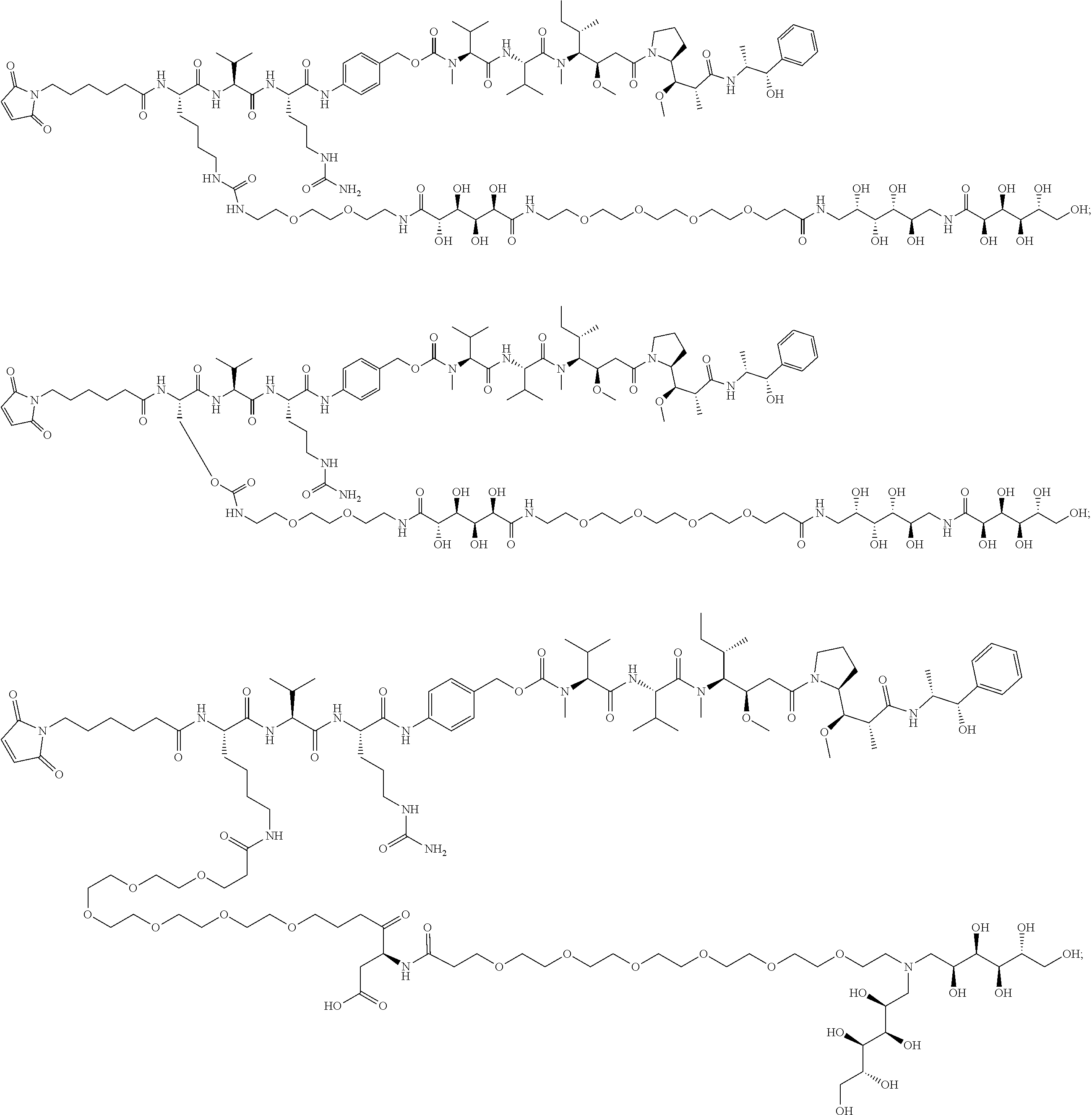

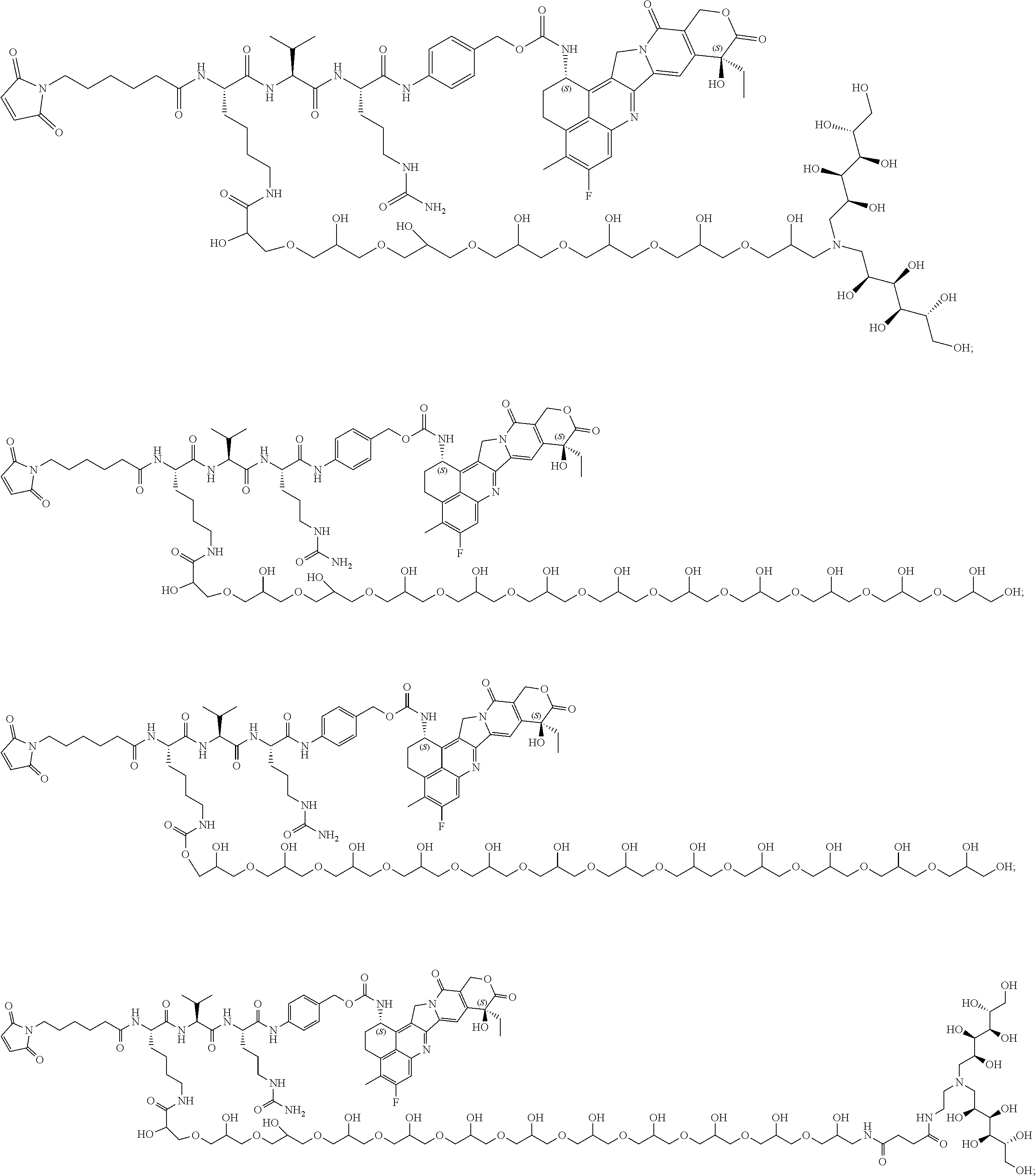

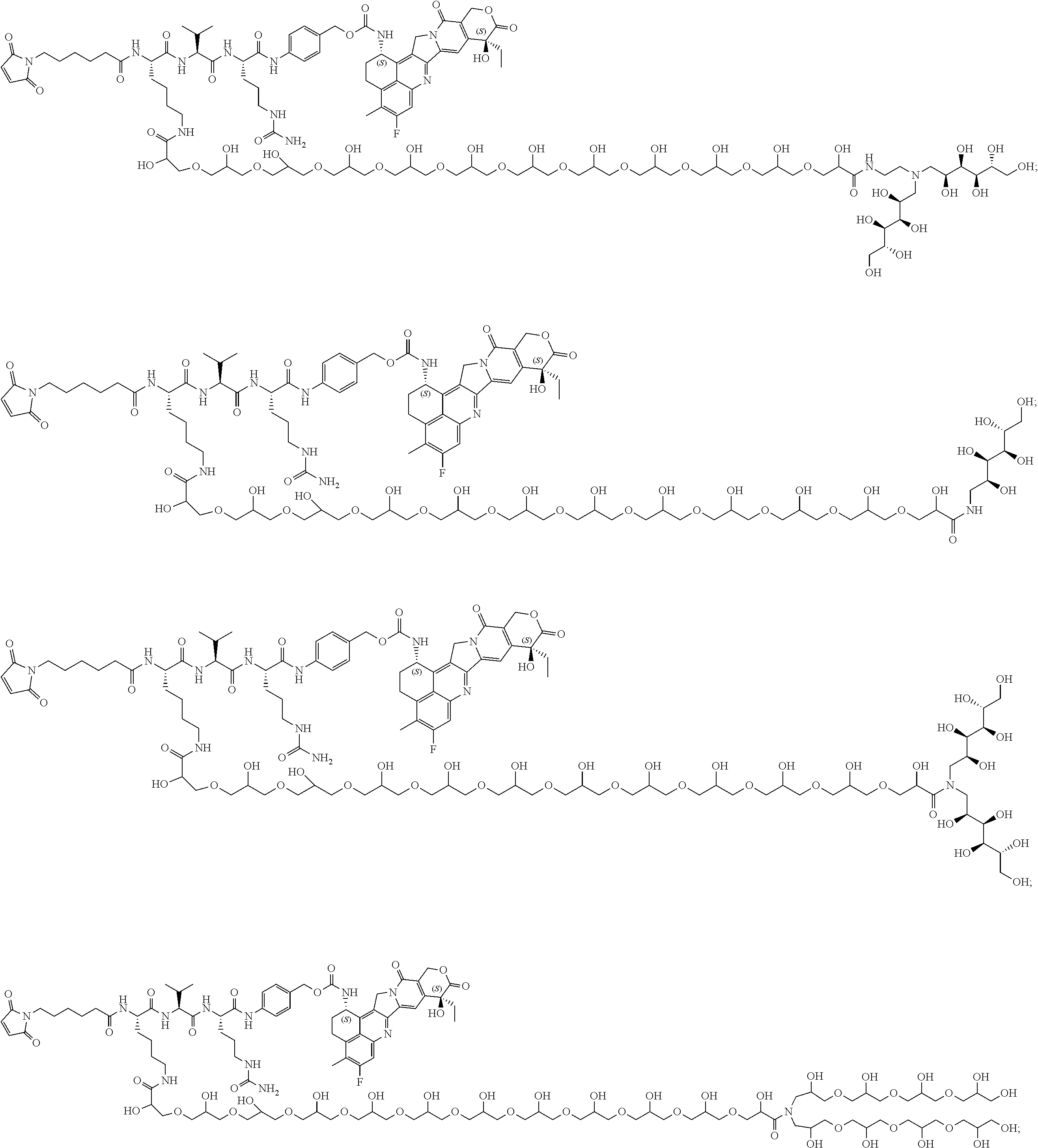

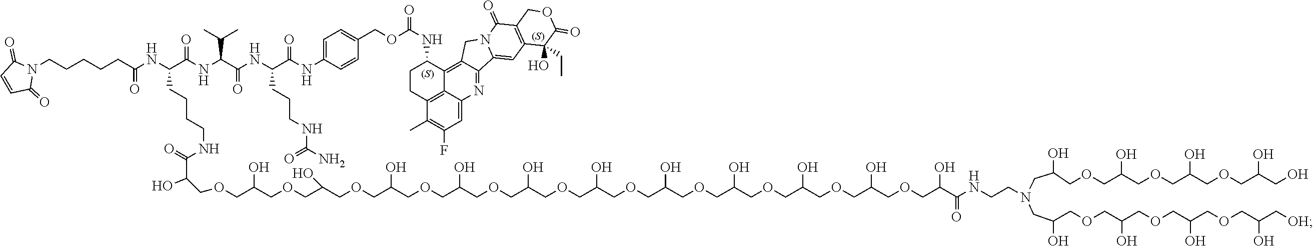

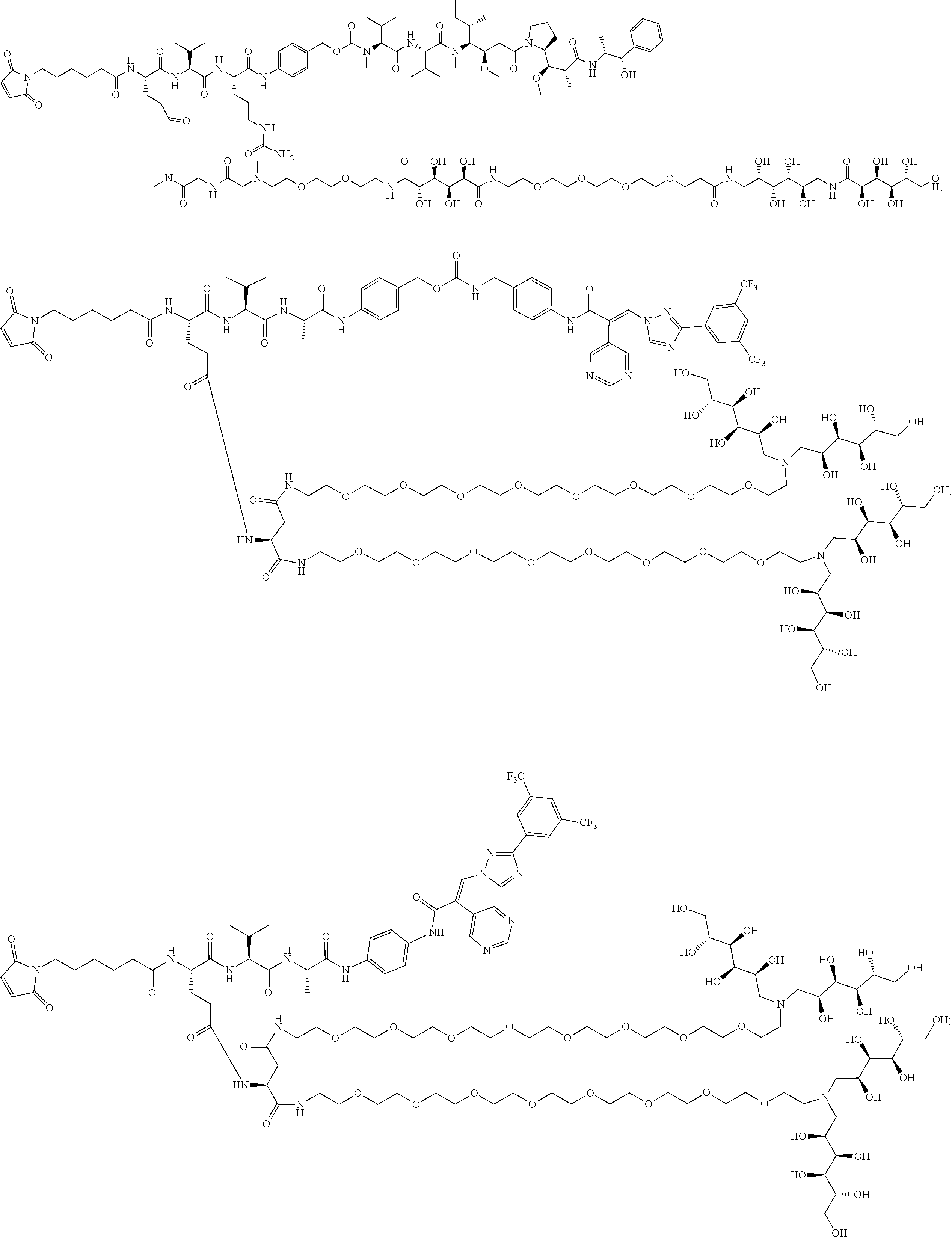

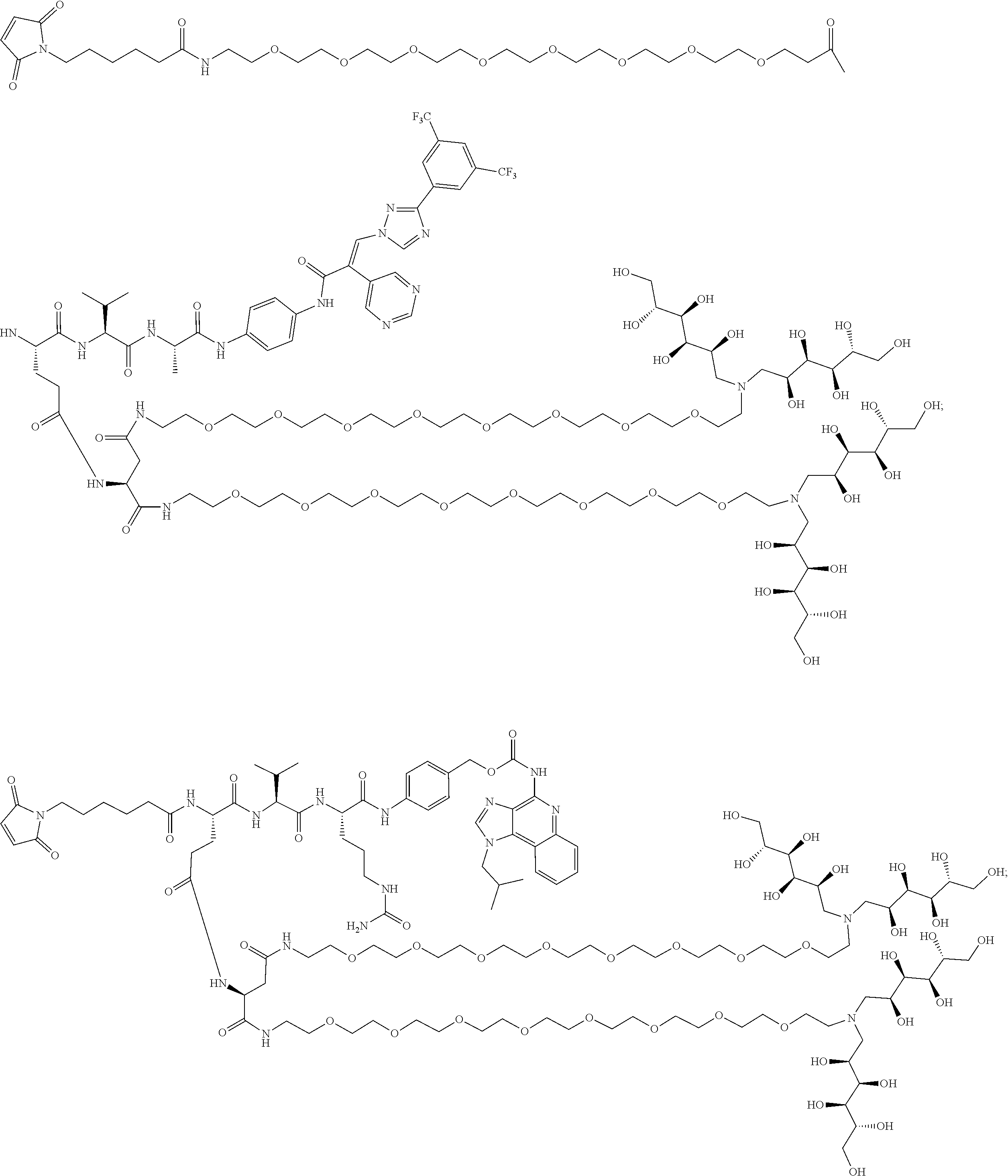

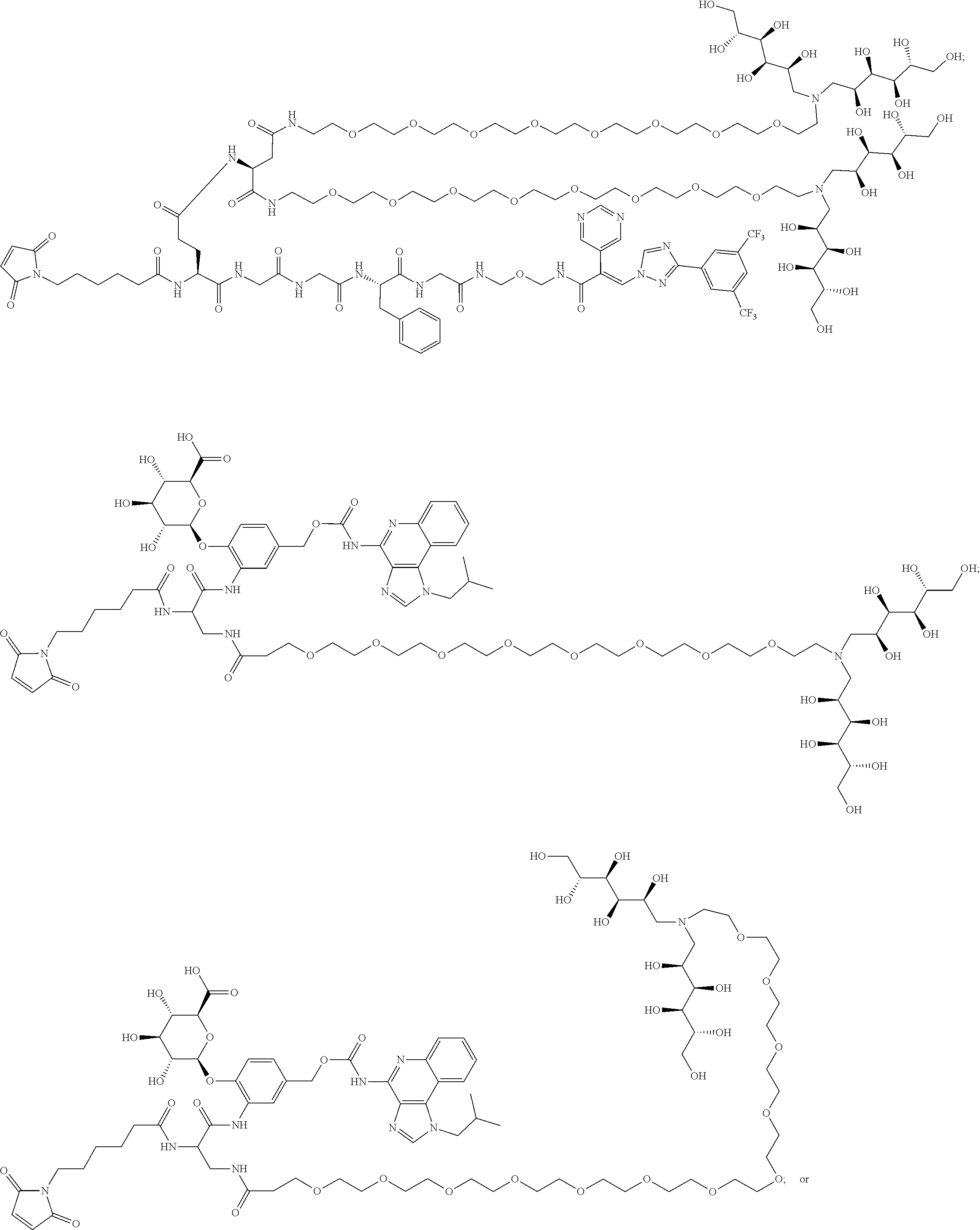

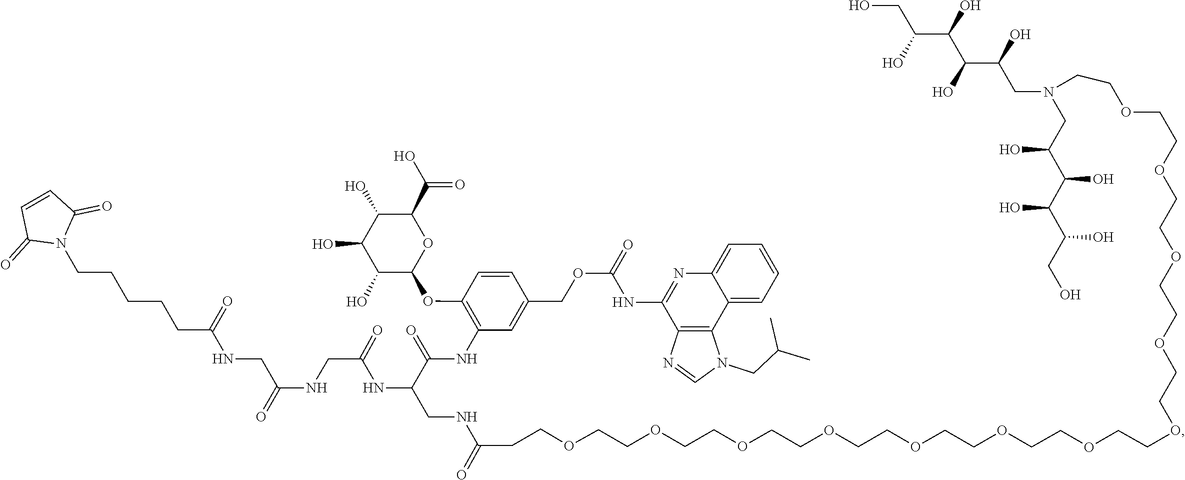 [SMILES: CC(C)Cn1cnc2c(NC(=O)OCc3ccc(O[C@@H]4O[C@H](C(=O)O)[C@@H](O)[C@H](O)[C@H]4O)c(NC(=O)C(CNC(=O)CCOCCOCCOCCOCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)NC(=O)CNC(=O)CNC(=O)CCCCCN4C(=O)C=CC4=O)c3)nc3ccccc3c21]

or a stereoisomer thereof.

Embodiment 137. A conjugate, comprising the drug-linker compound of any one of Embodiments 109-136, wherein the drug-linker compound is attached to a binding agent (e.g., SLITRK6 binding agent).

Embodiment 138. The conjugate of Embodiment 137, wherein an average drug loading (p^{load}) of the conjugate is from about 1 to about 8, about 2, about 4, about 6, about 8, about 10, about 12, about 14, about 16, about 3 to about 5, about 6 to about 8, or about 8 to about 16.

Embodiment 139. The conjugate of any one of Embodiments 137-138, selected from the following:

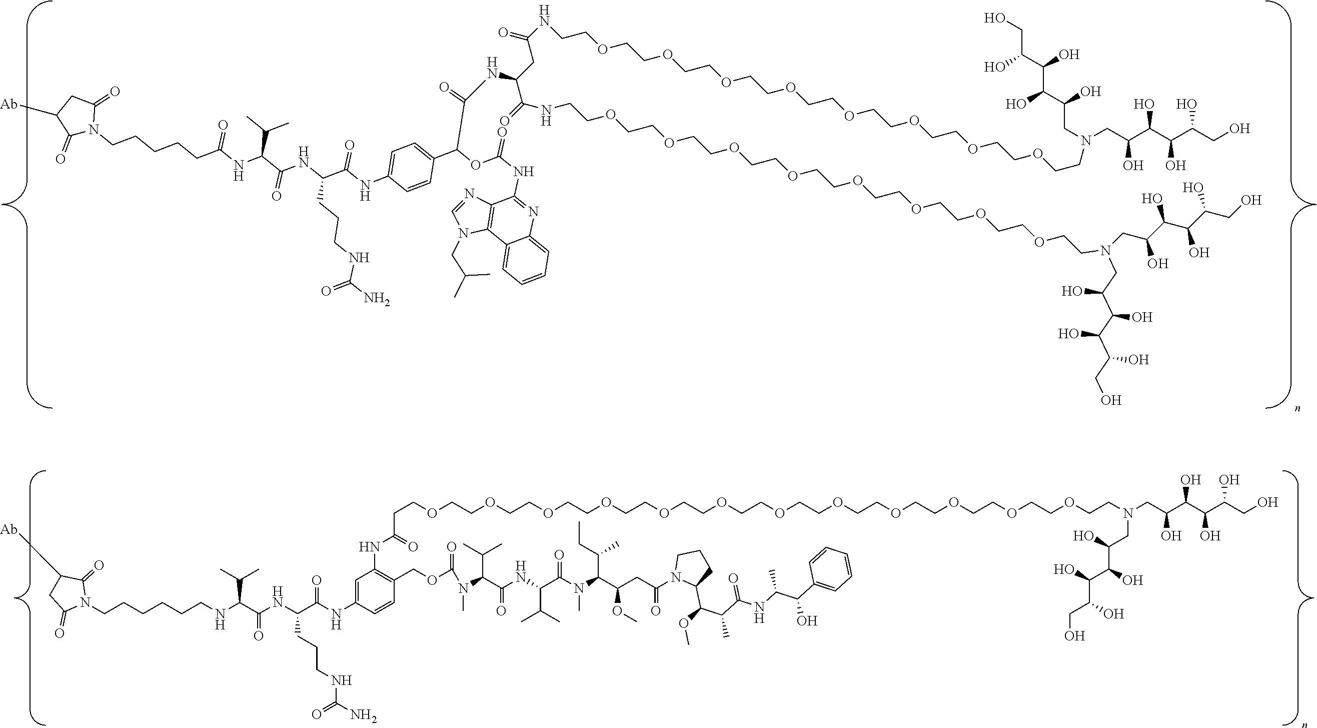

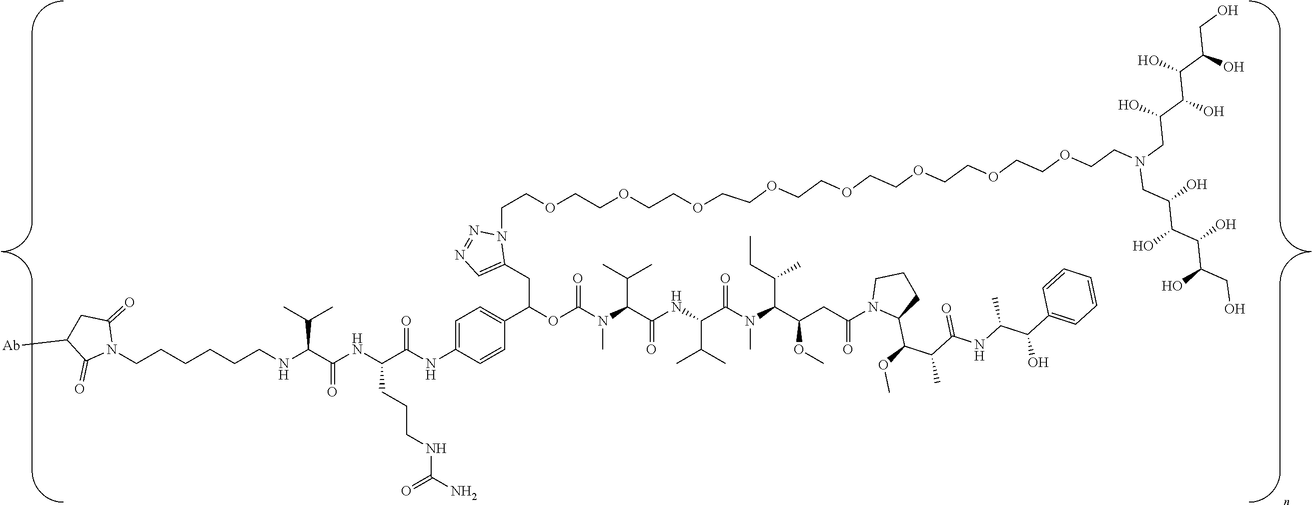

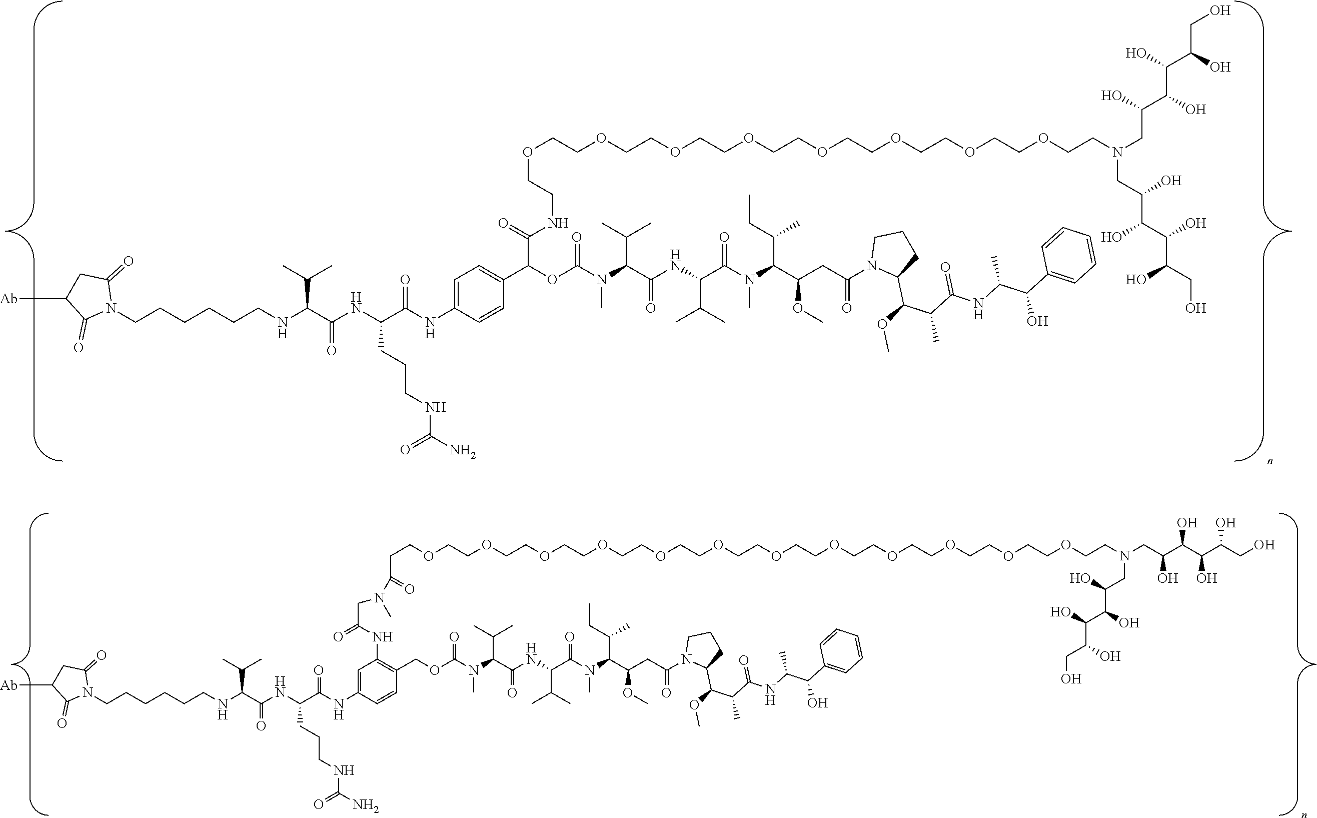

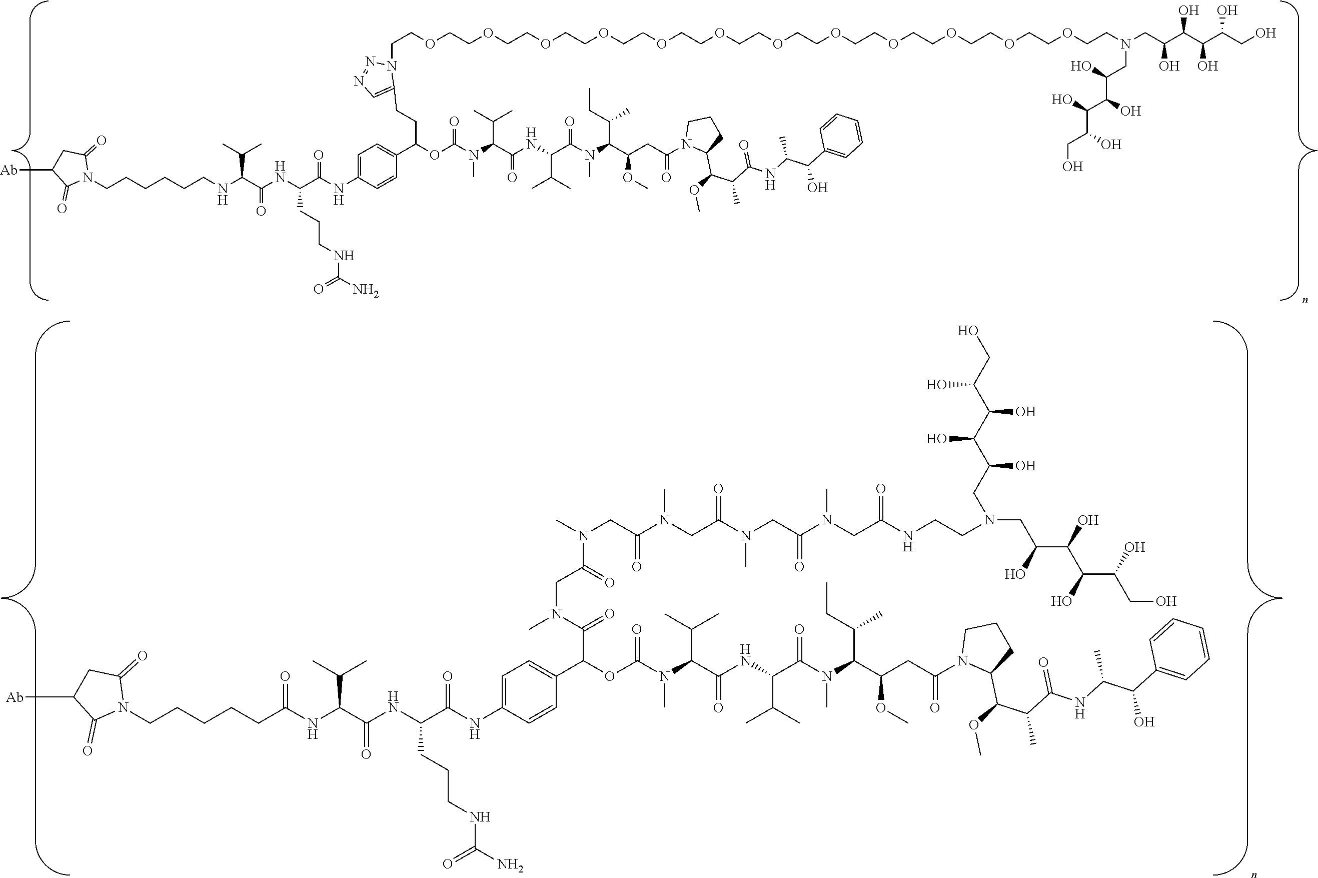

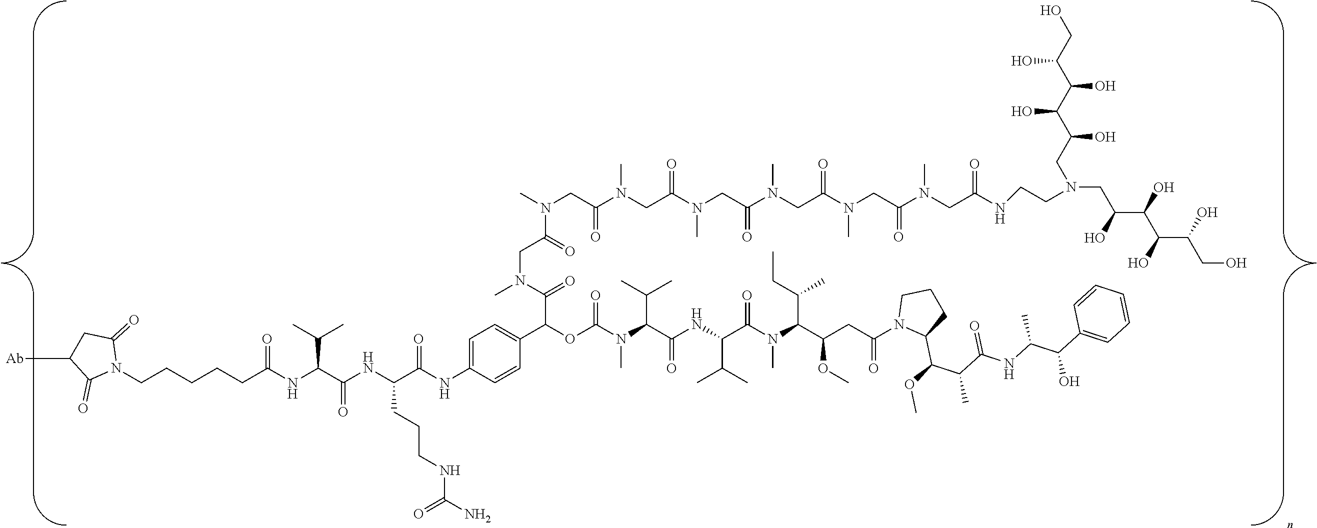

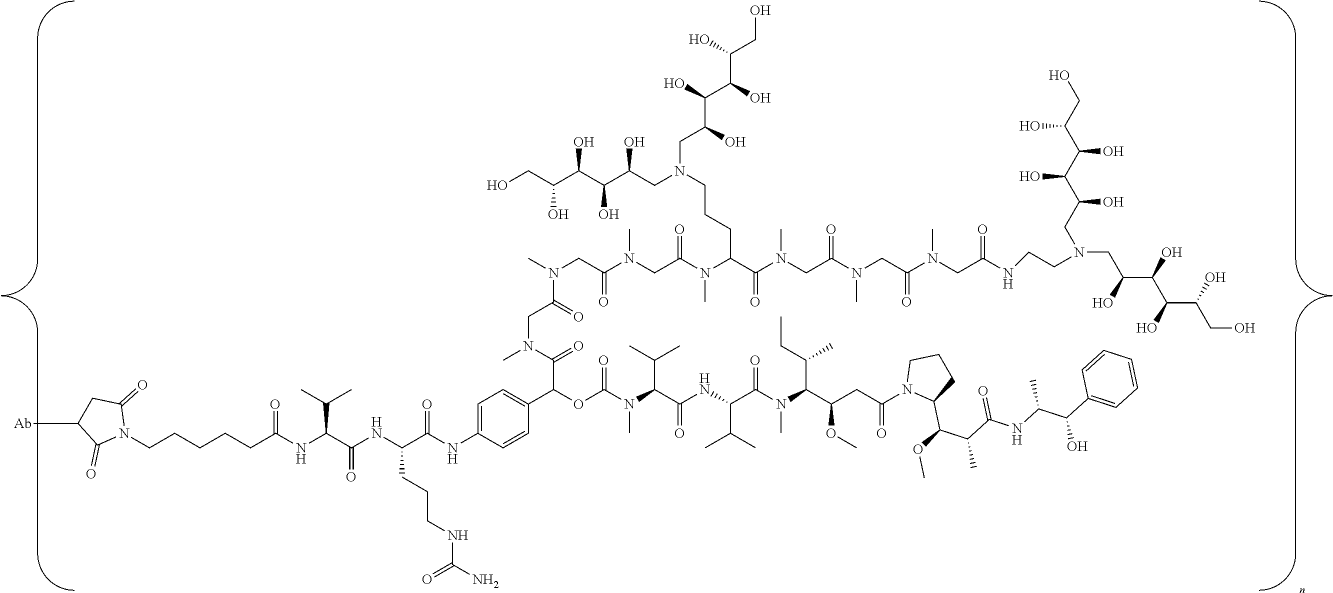

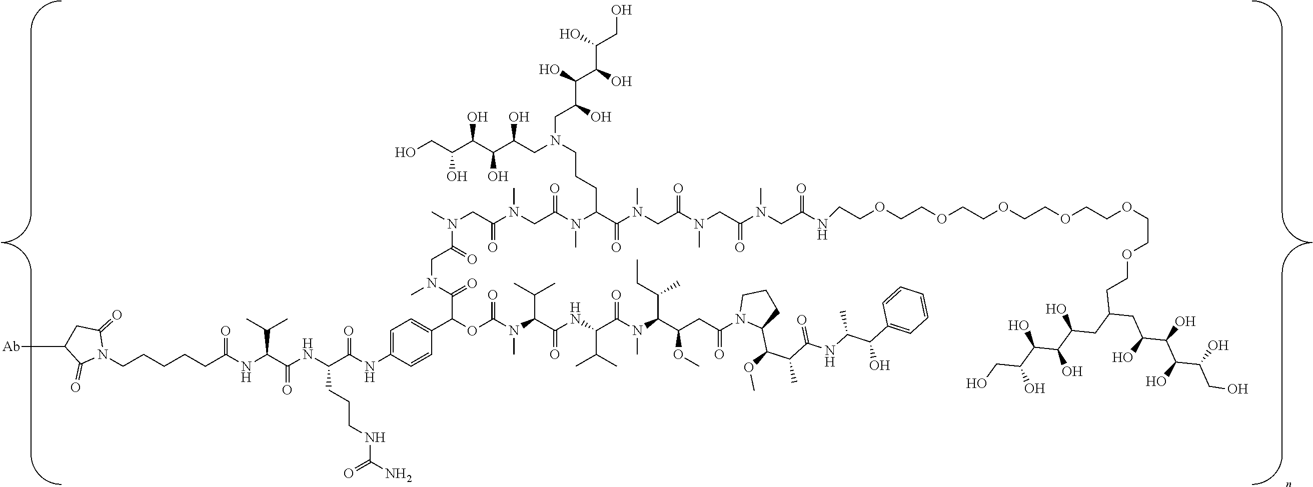

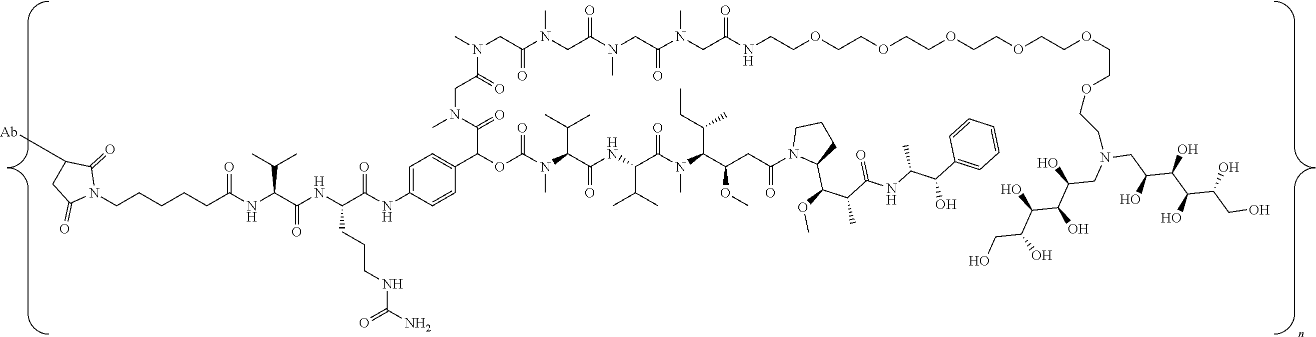

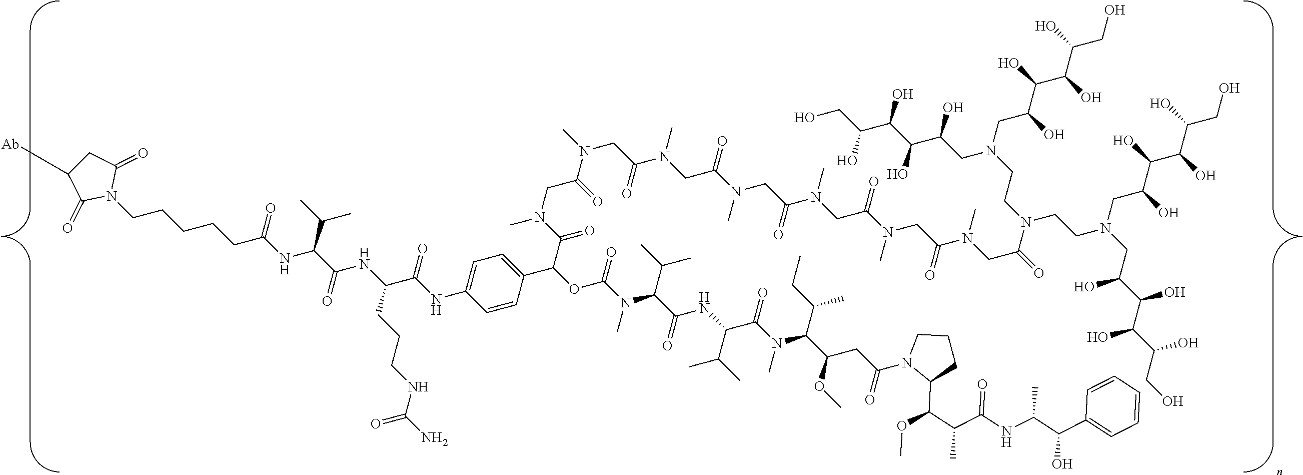

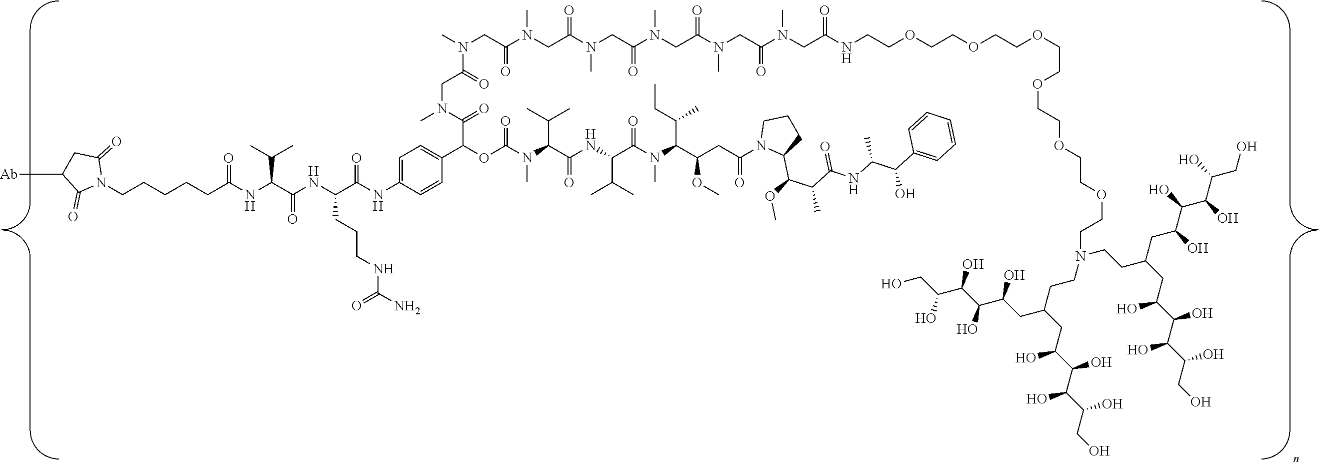

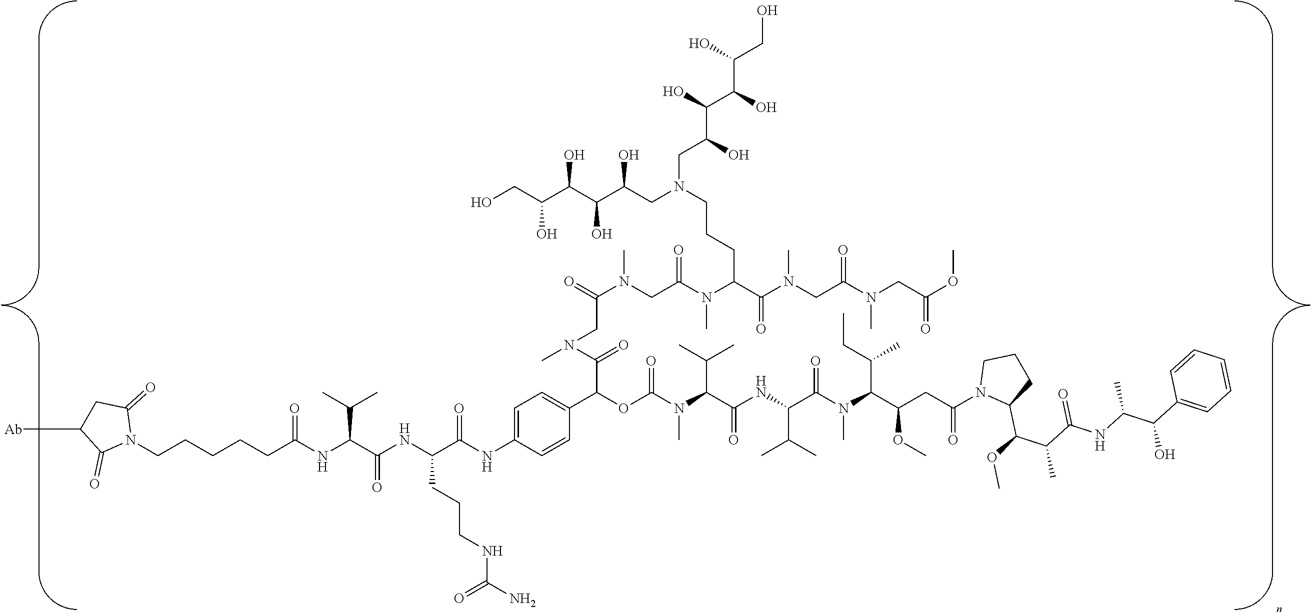

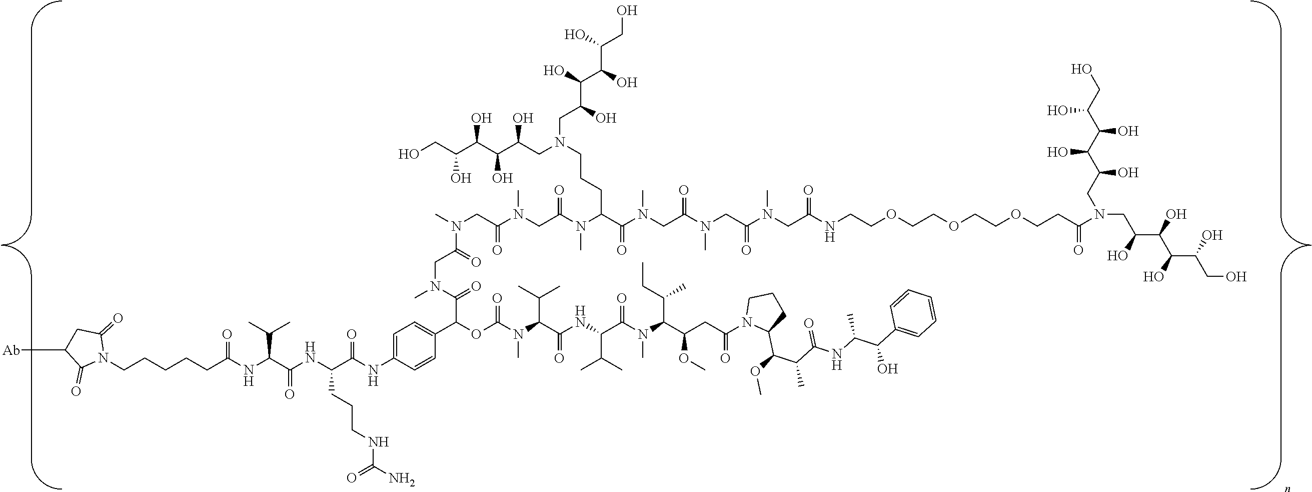

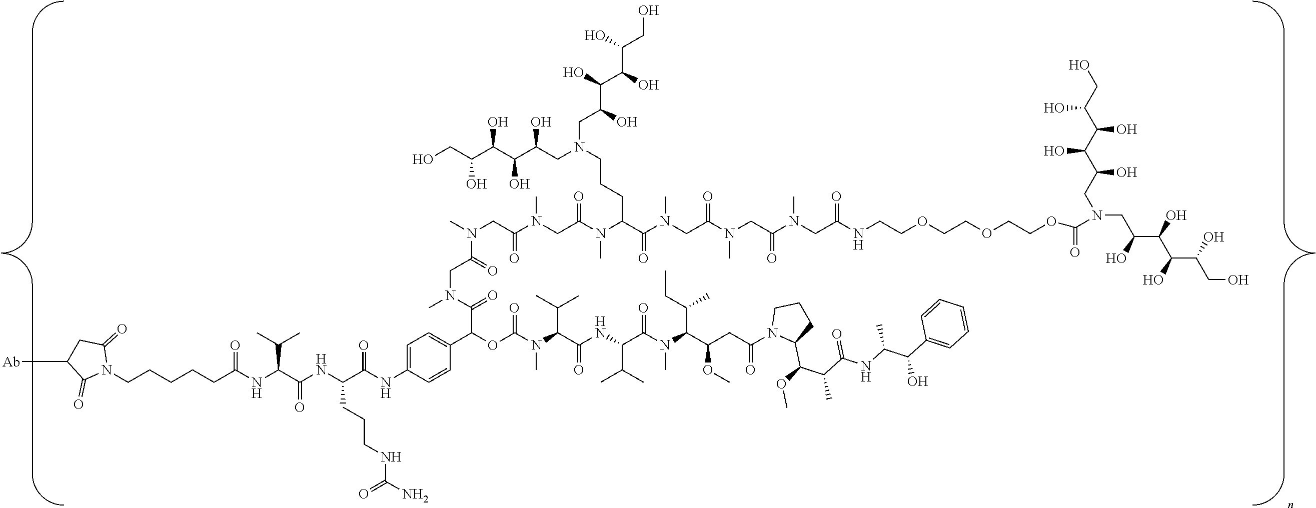

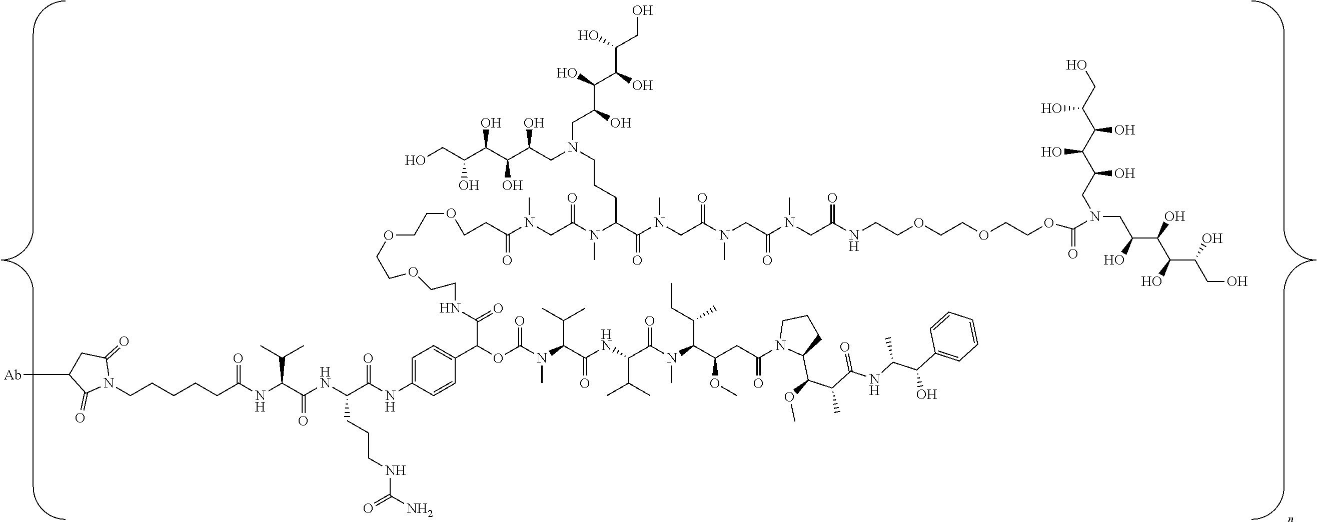

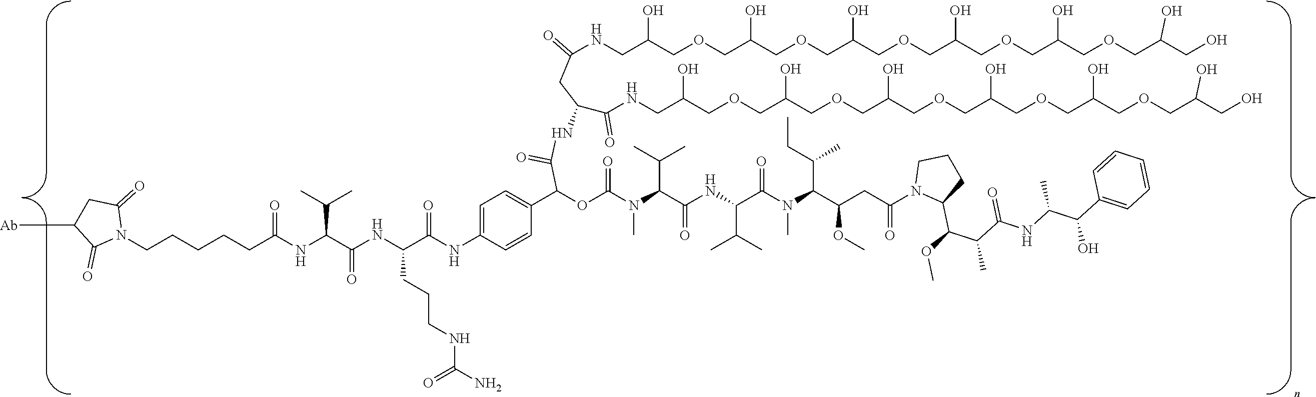

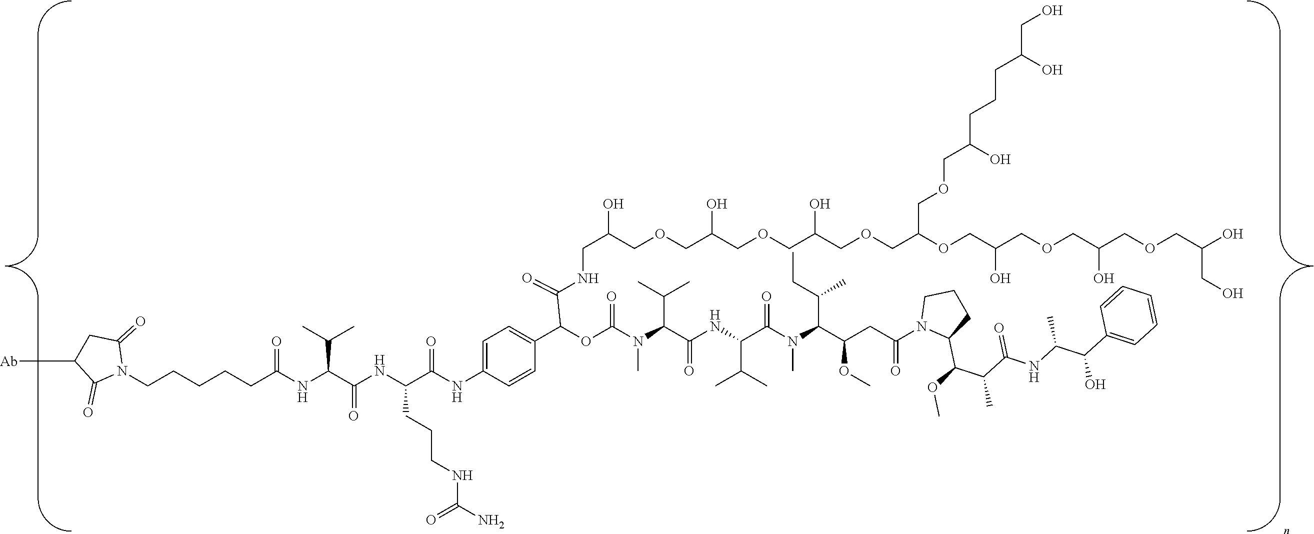

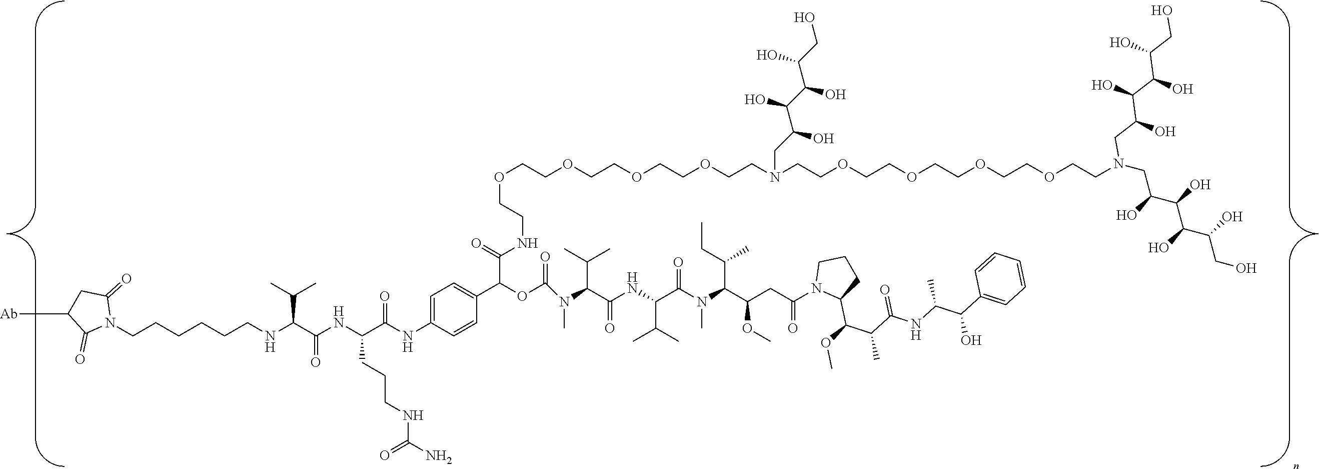

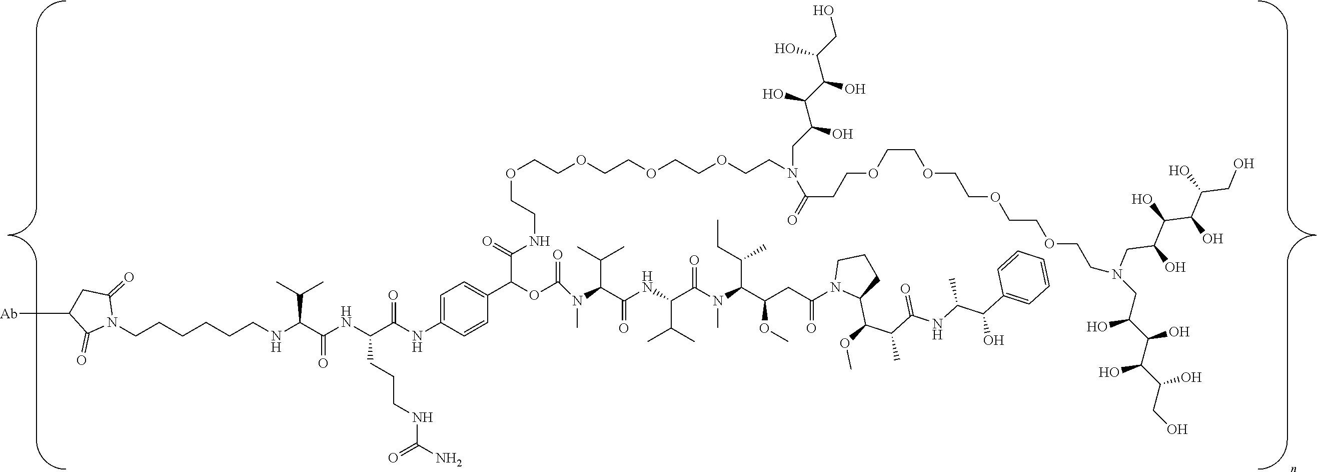

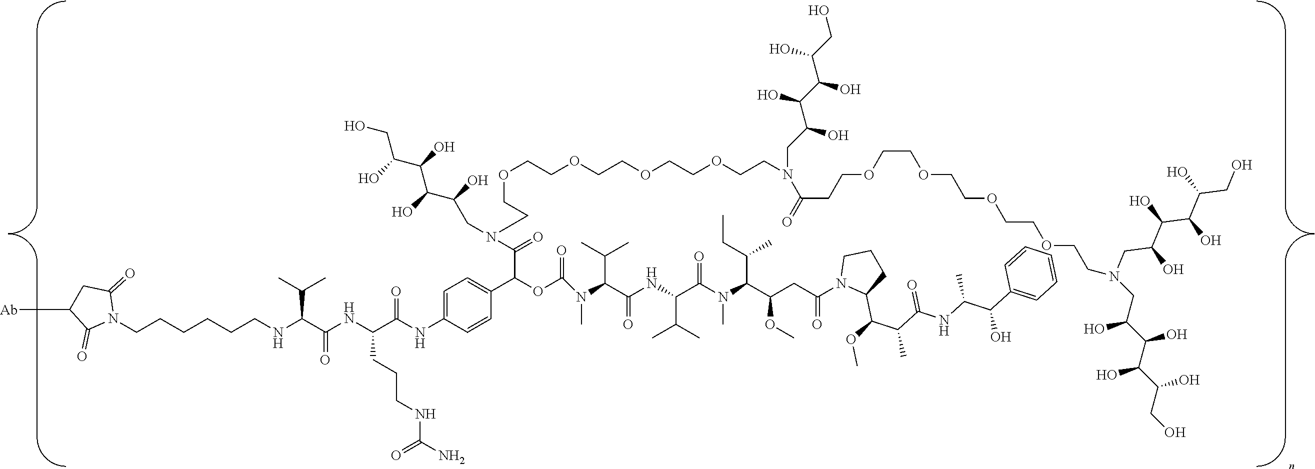

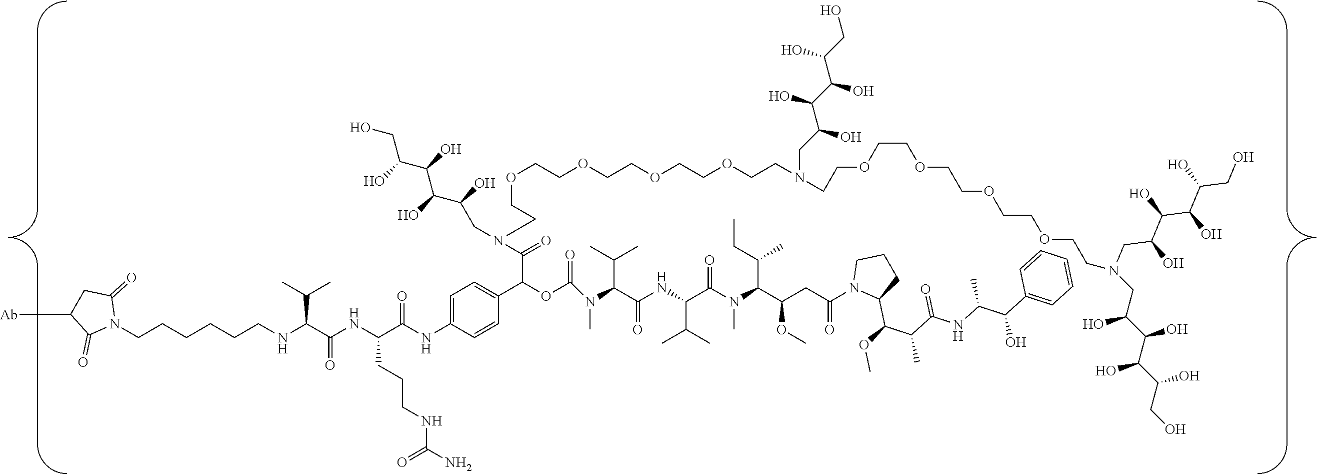

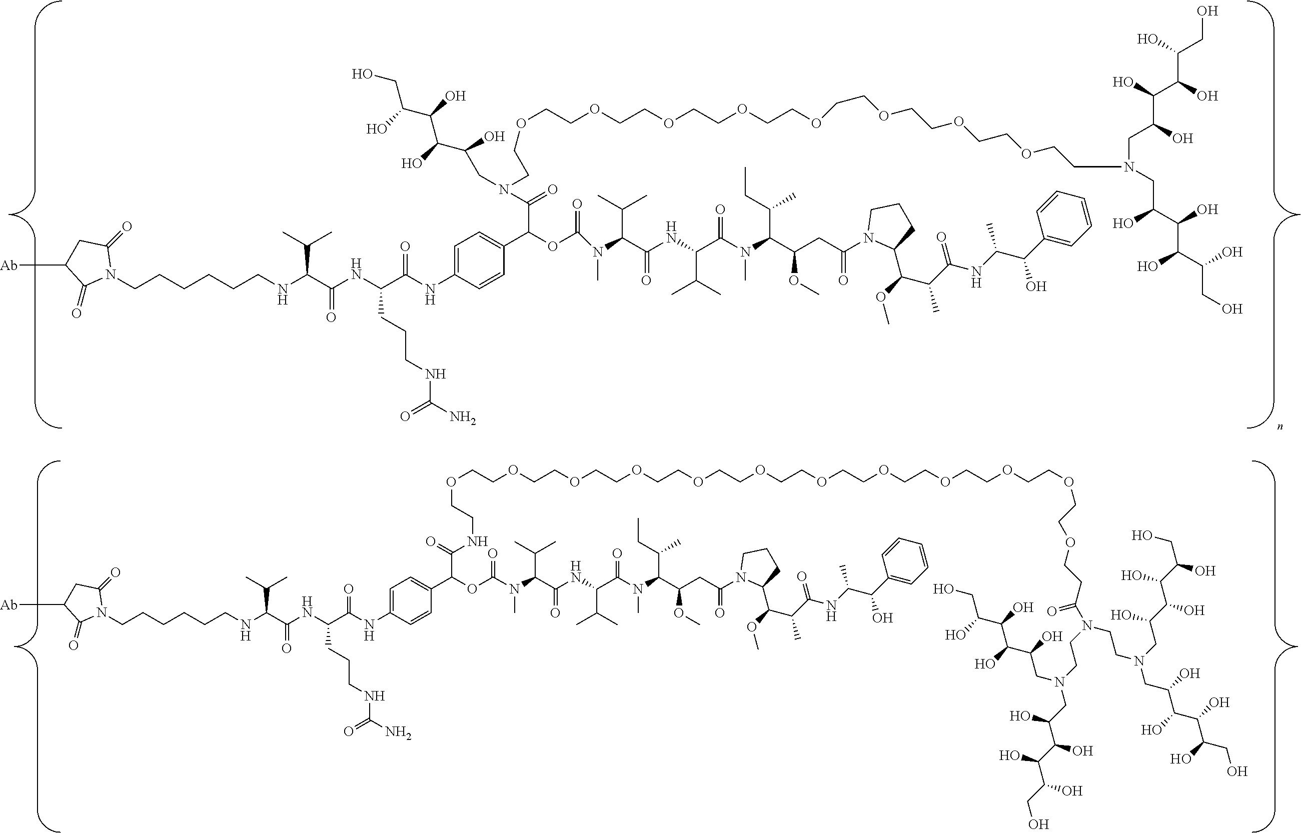

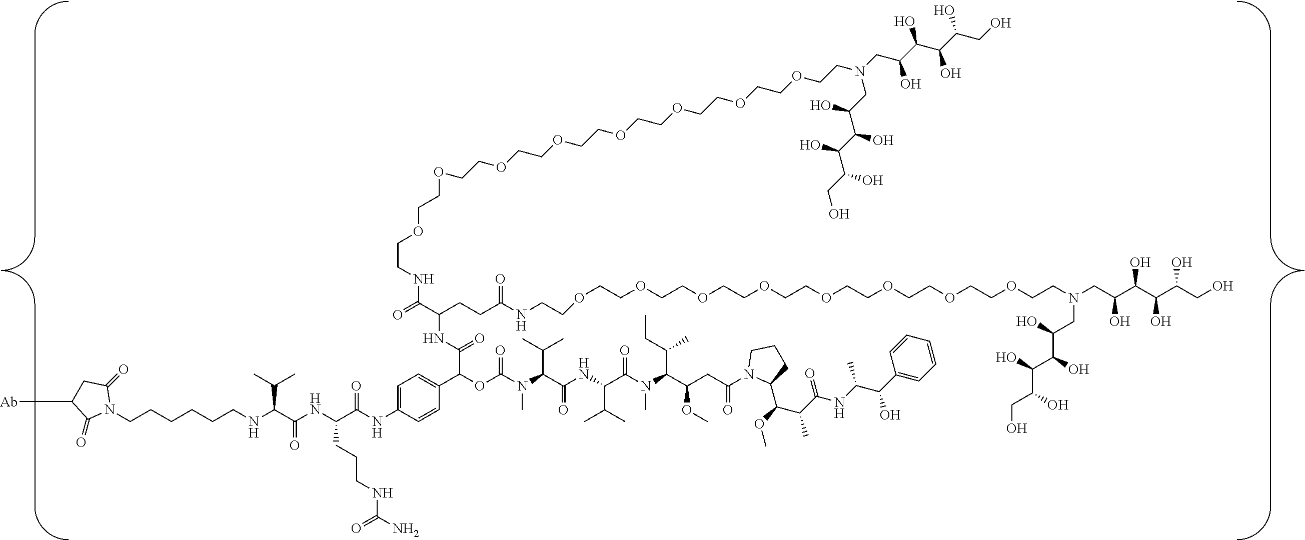

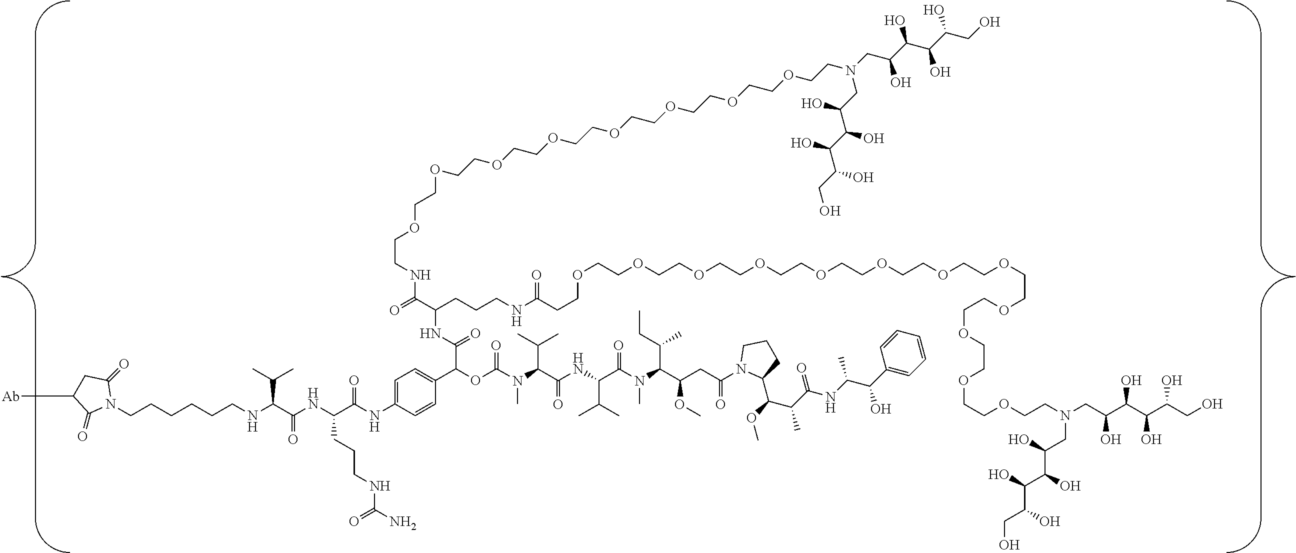

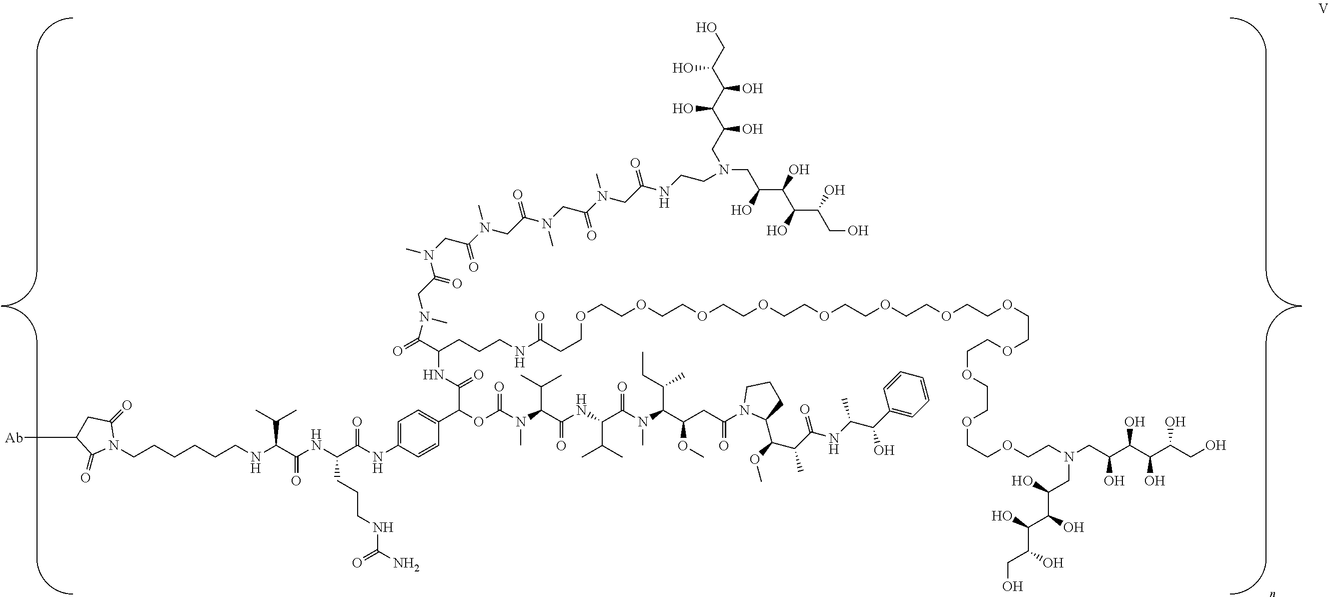

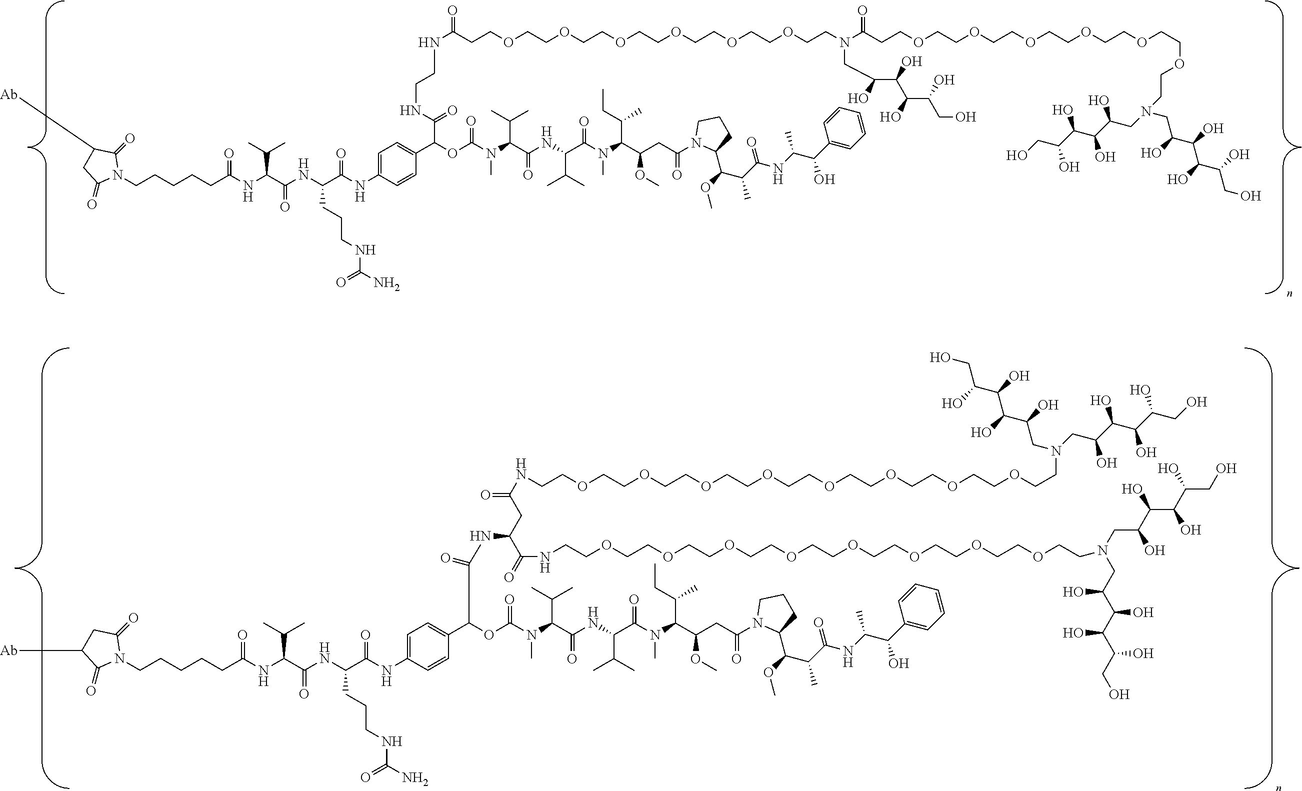

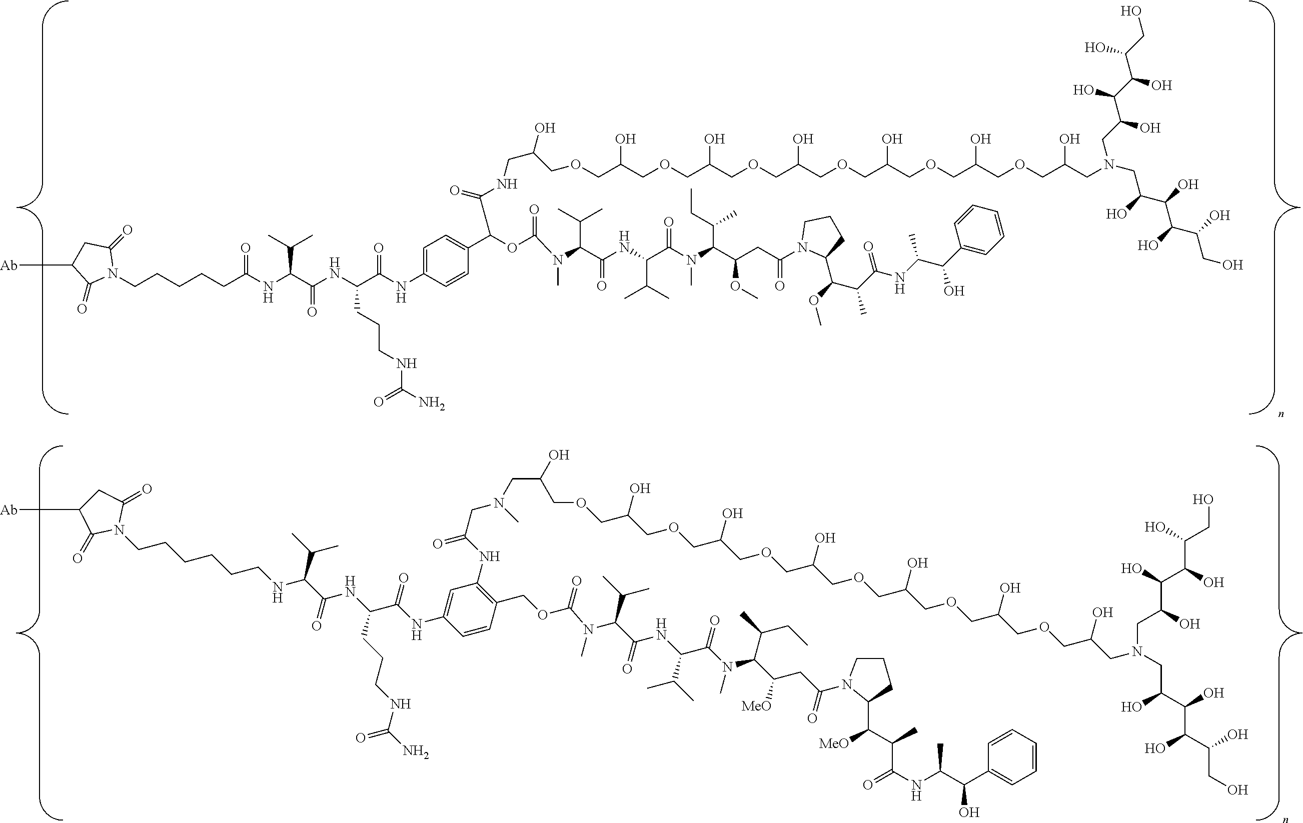

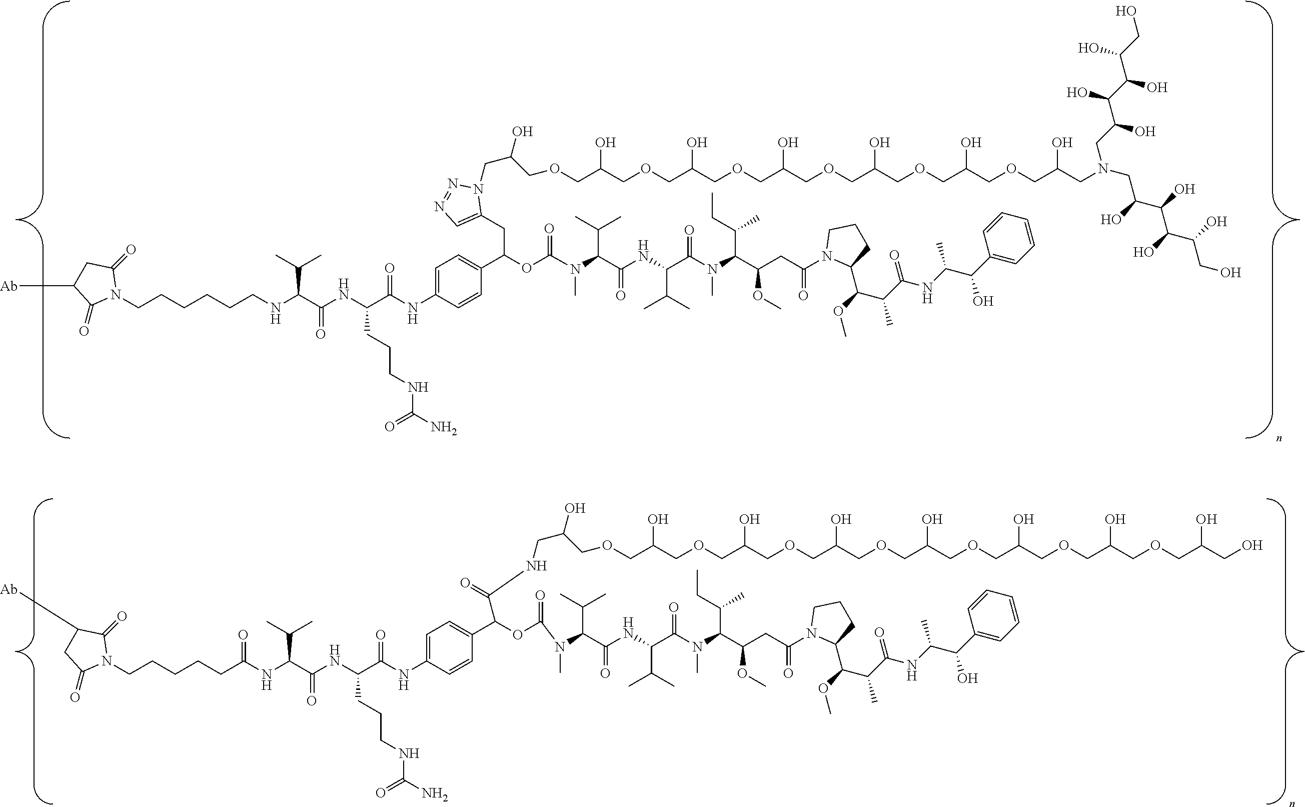

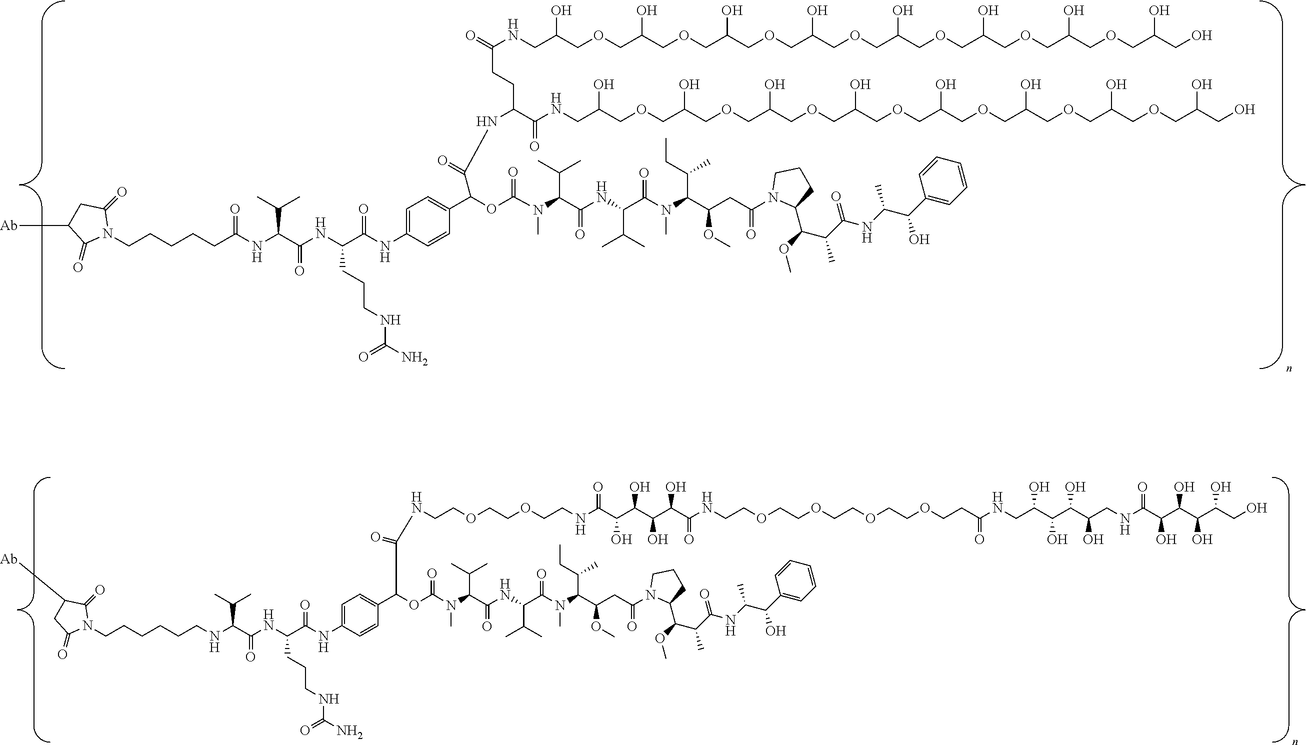

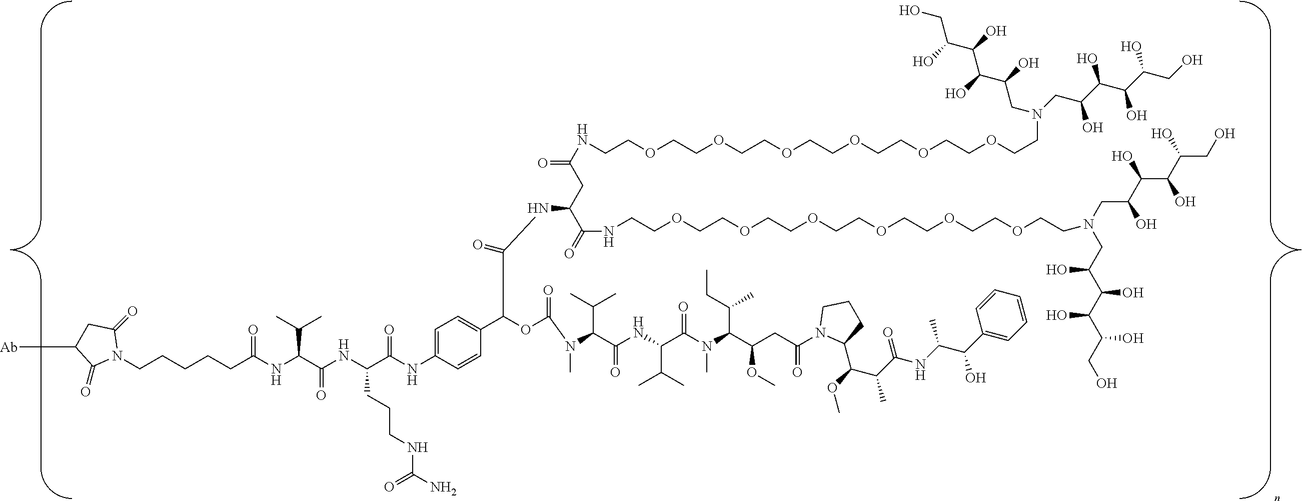

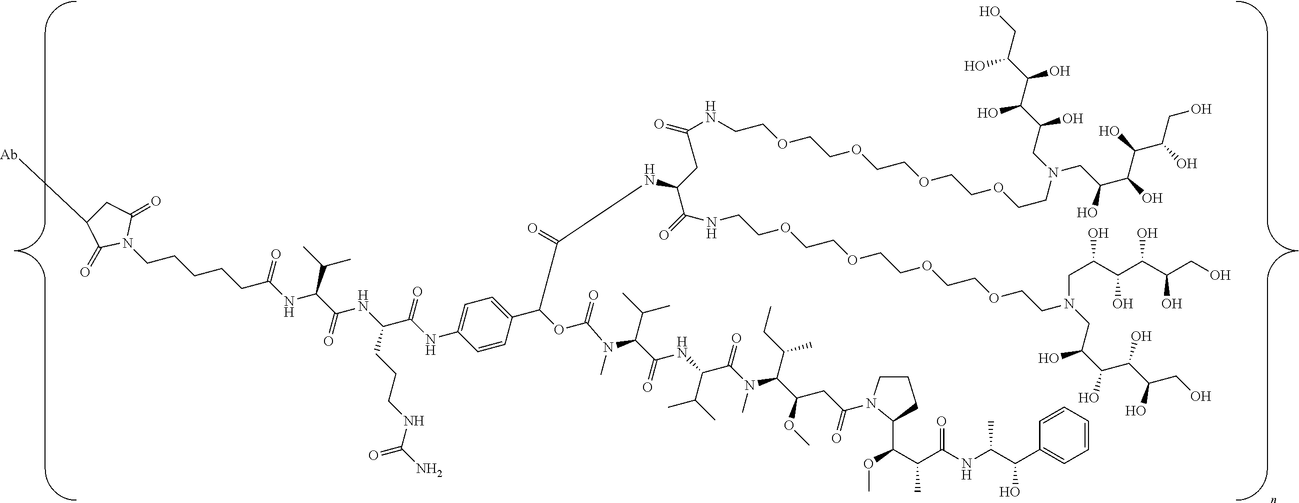

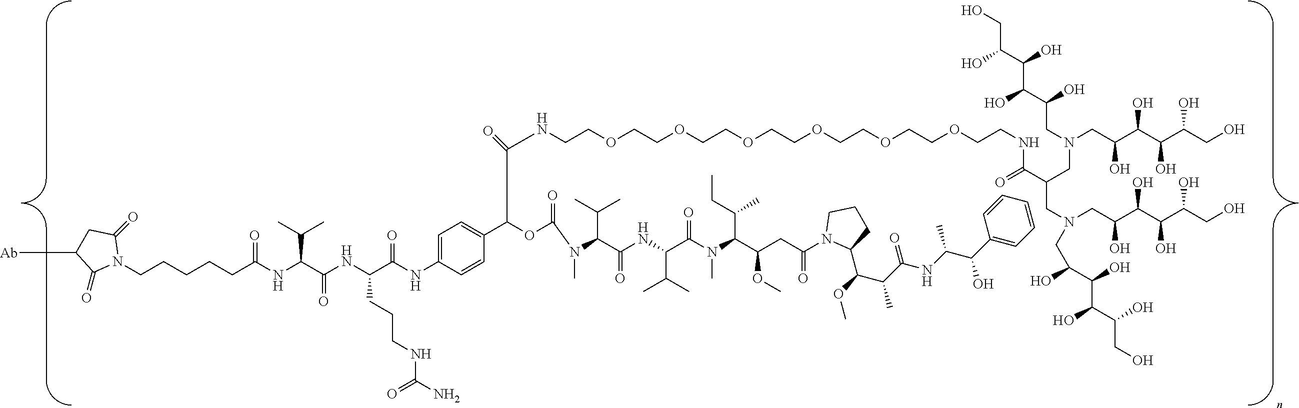

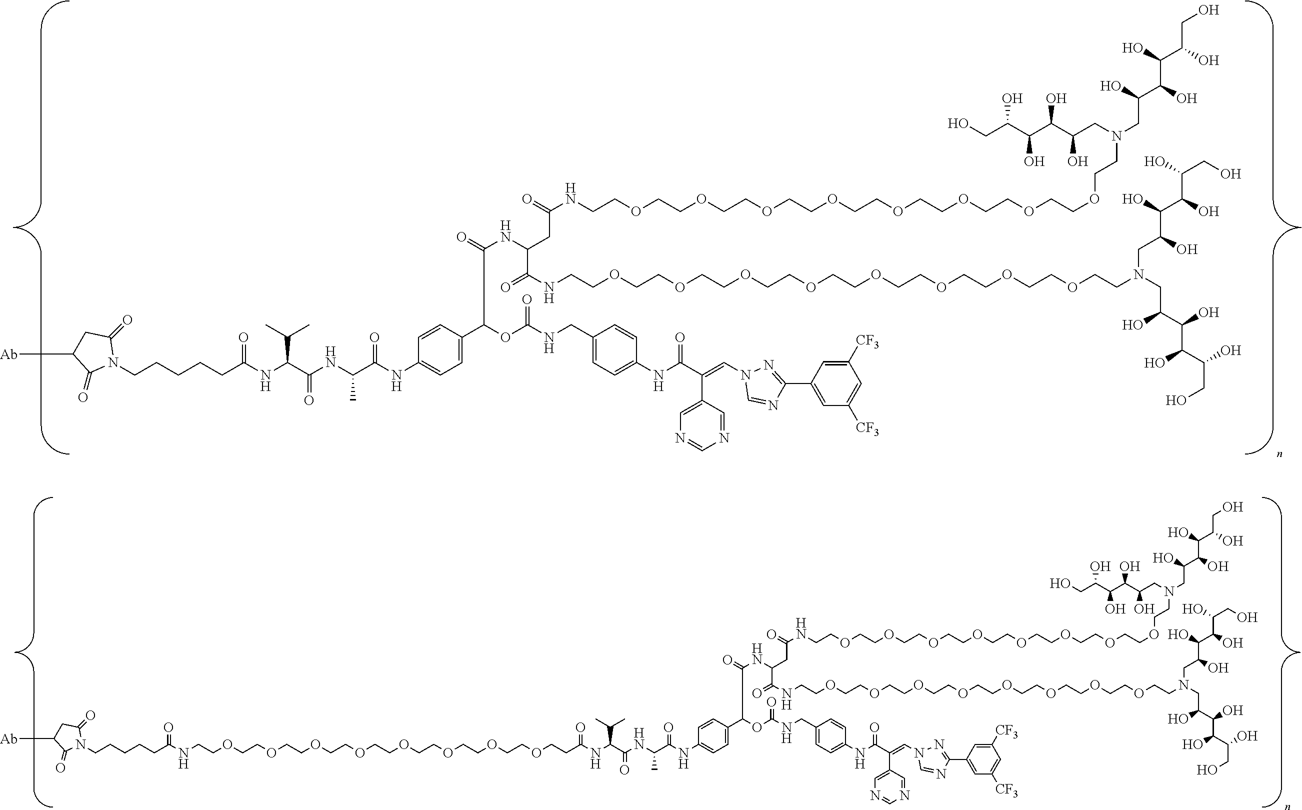

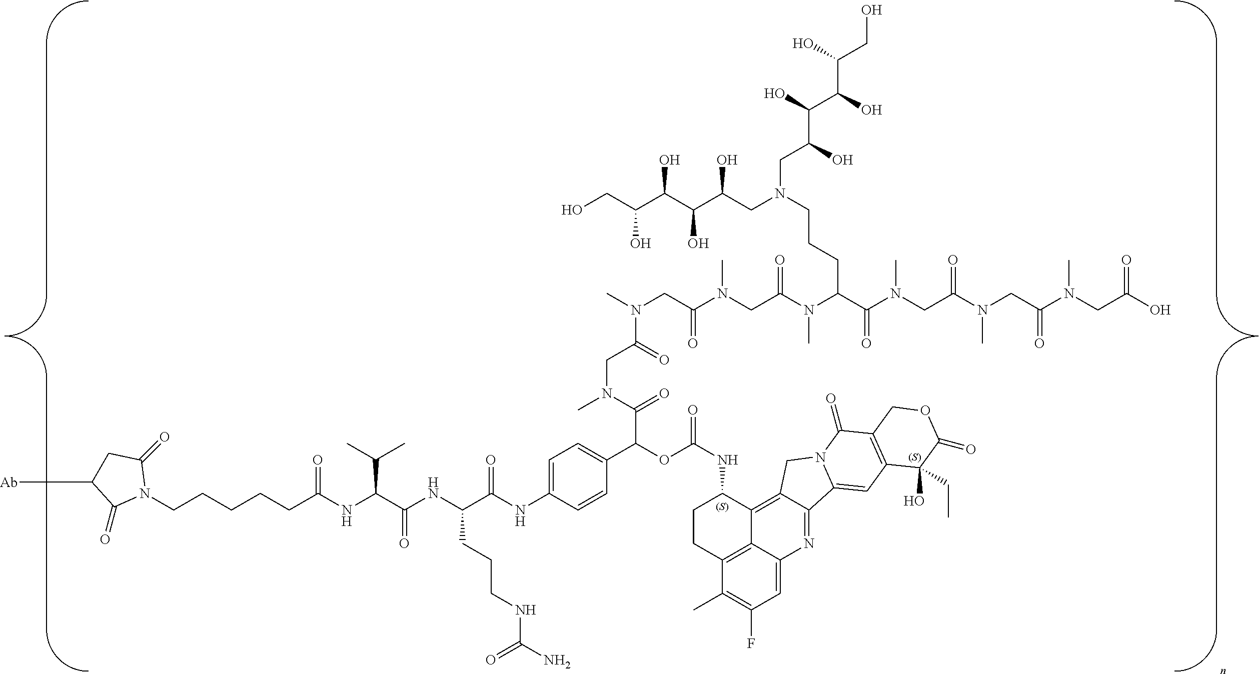

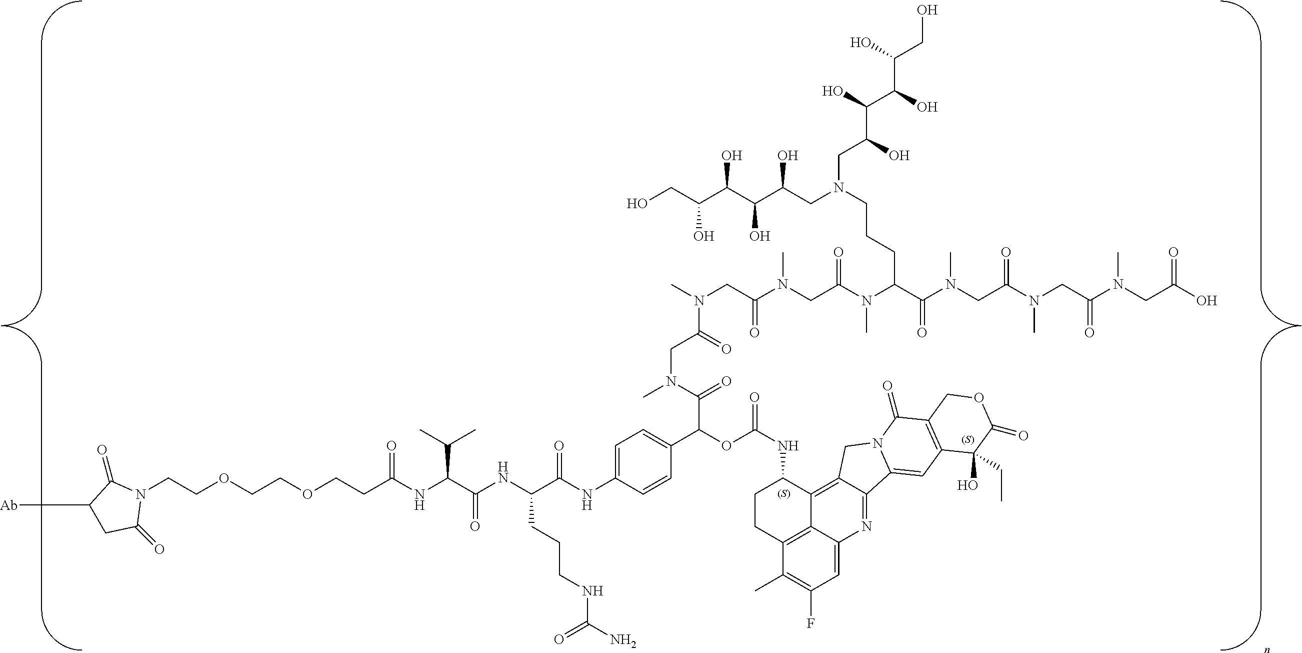

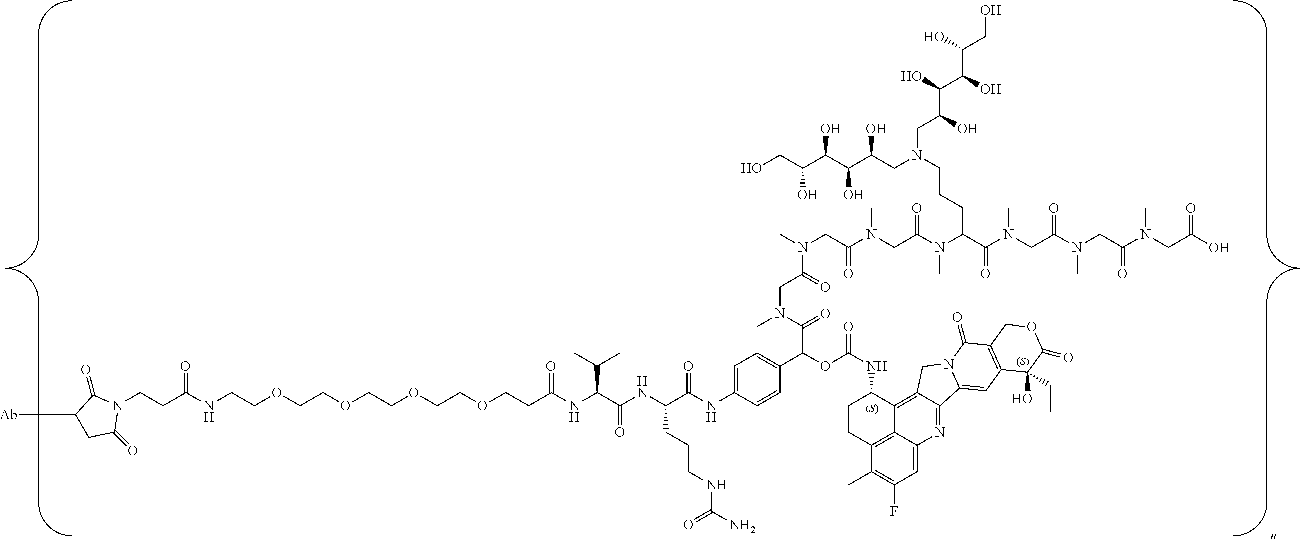

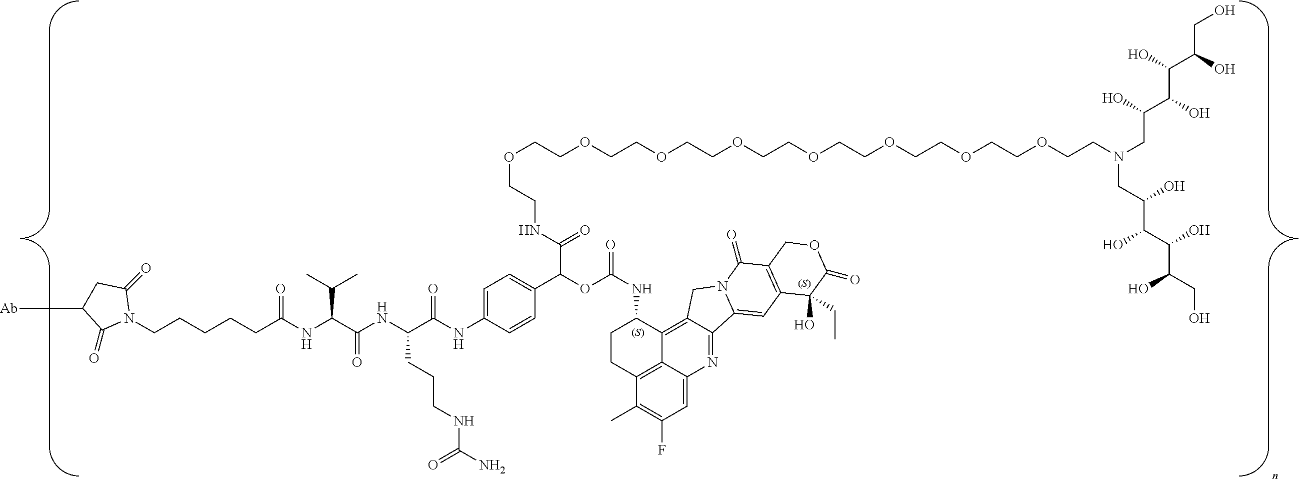

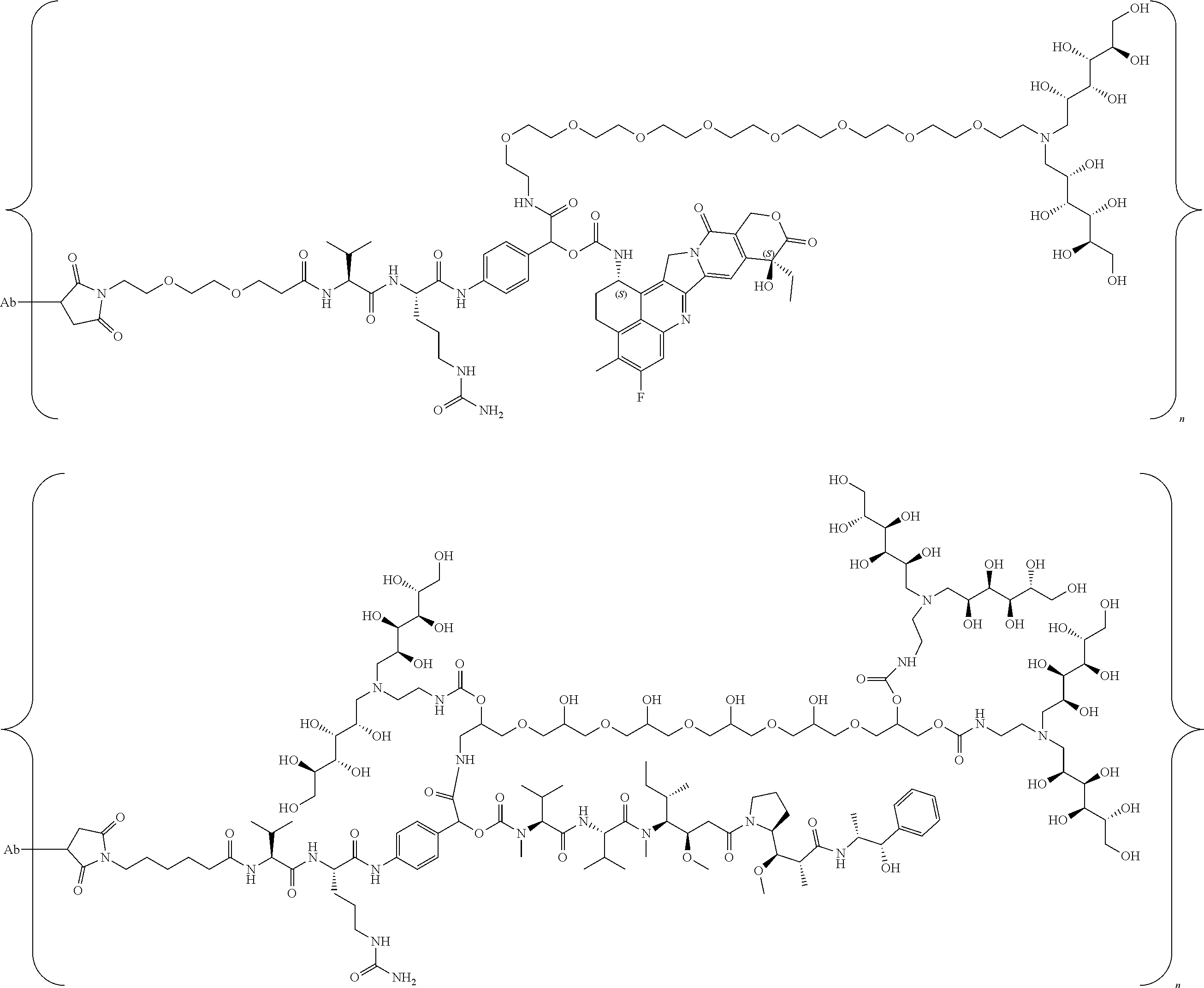

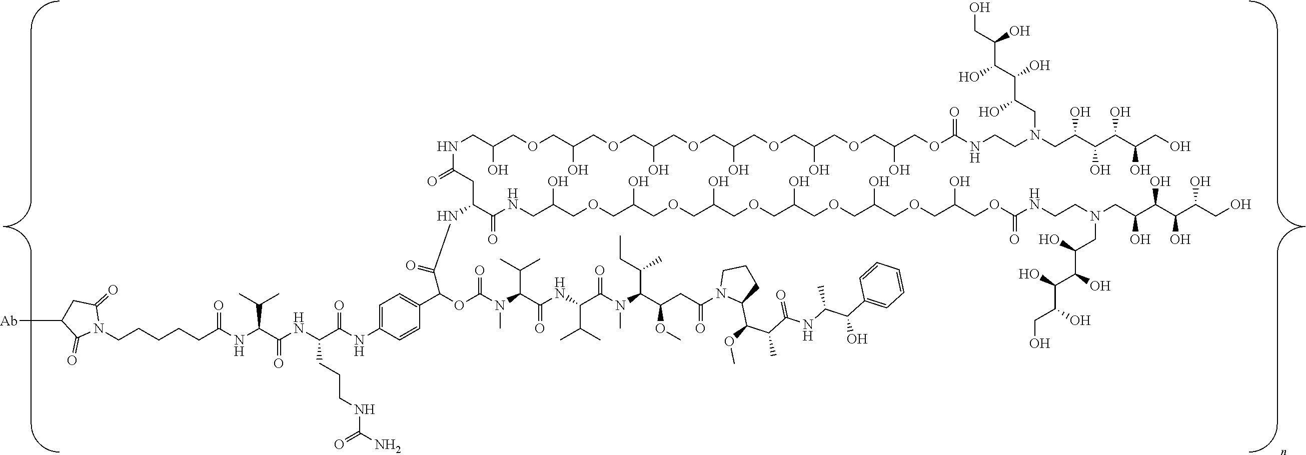

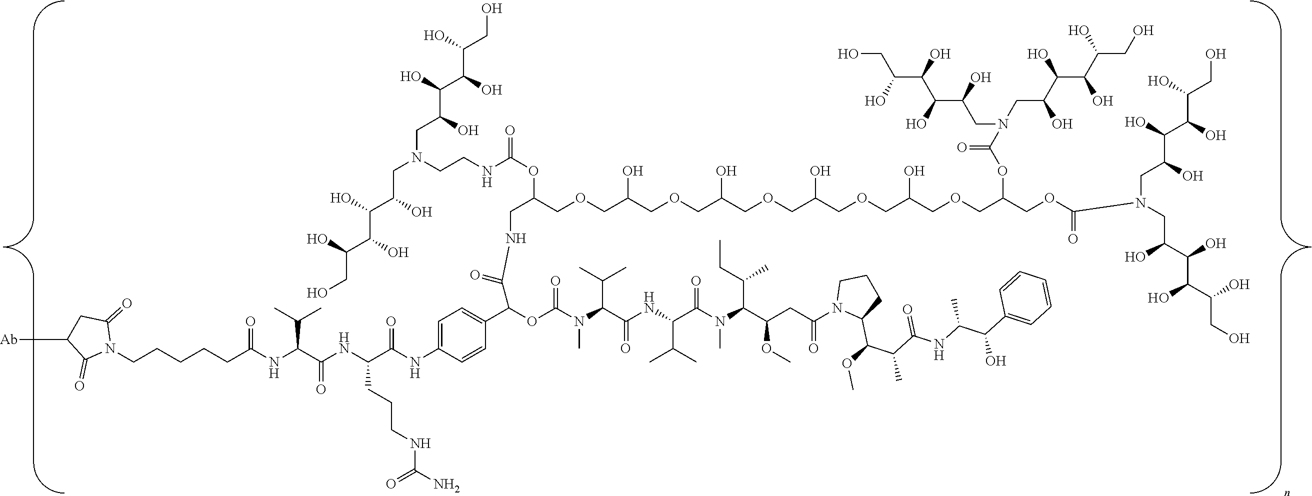

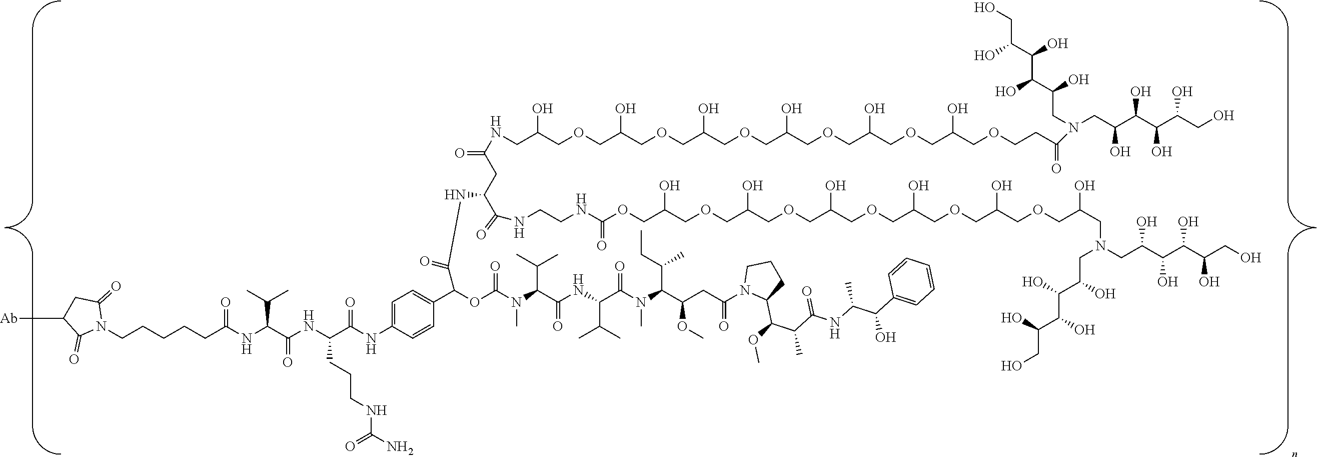

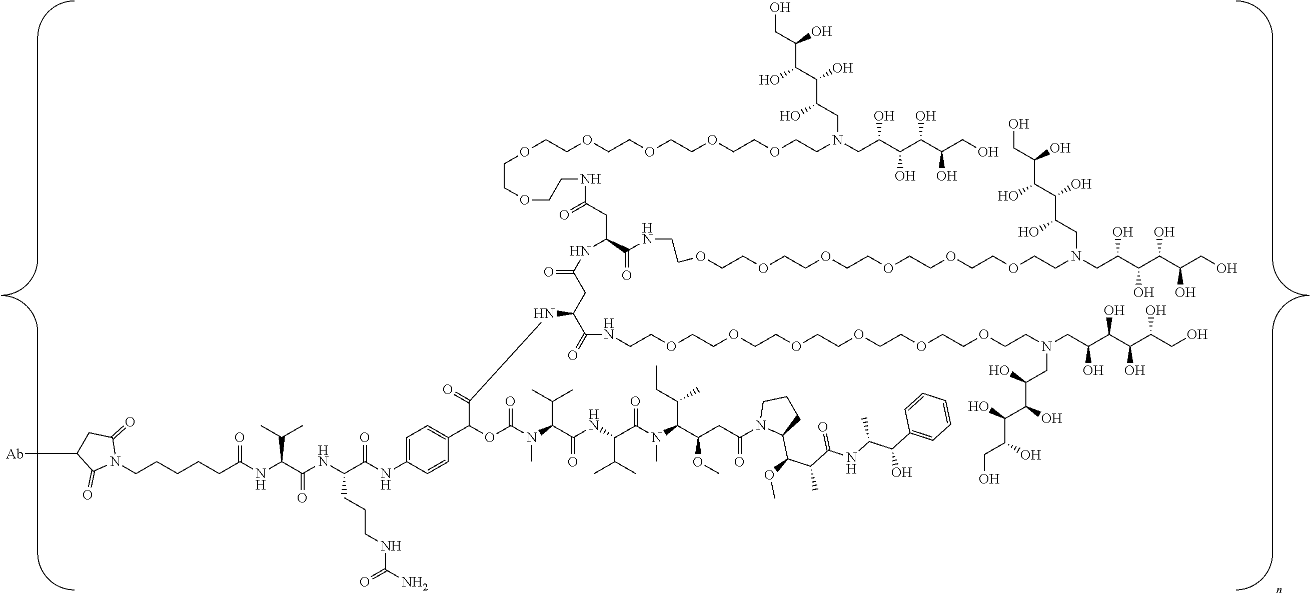

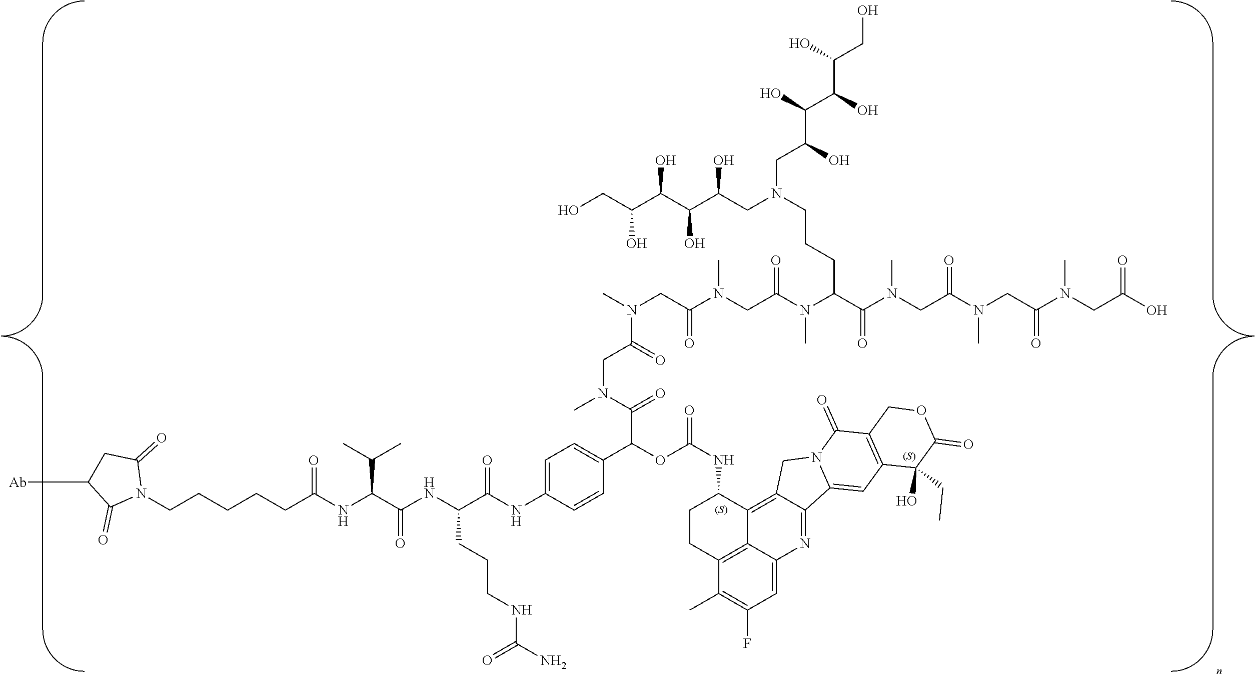

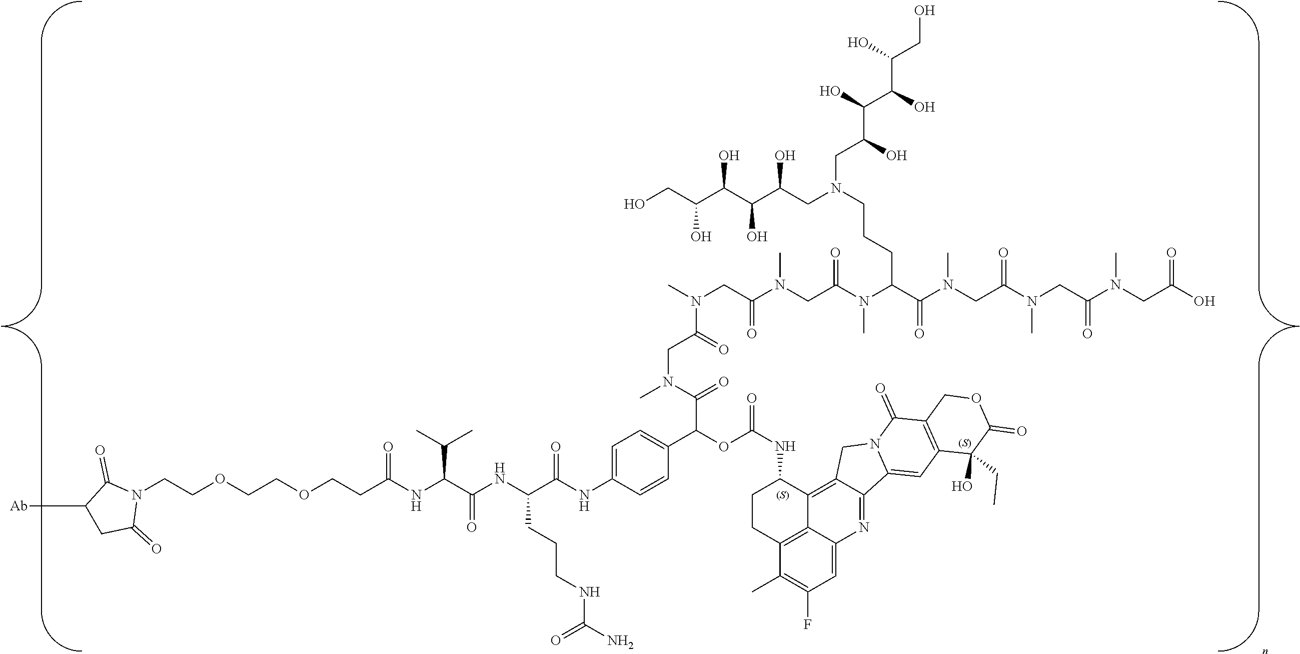

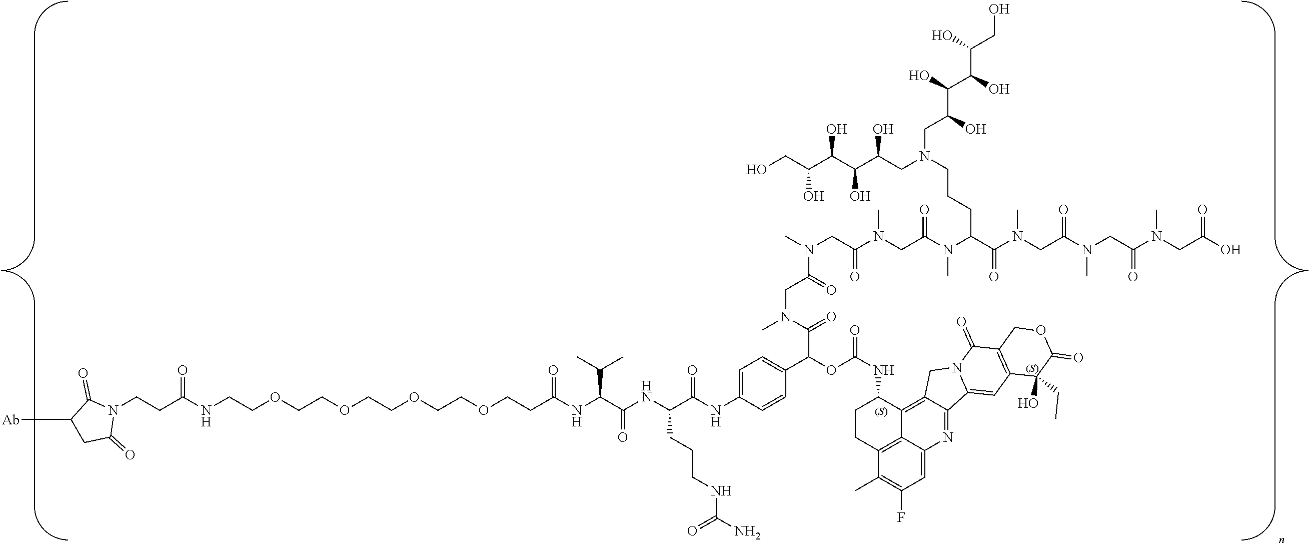

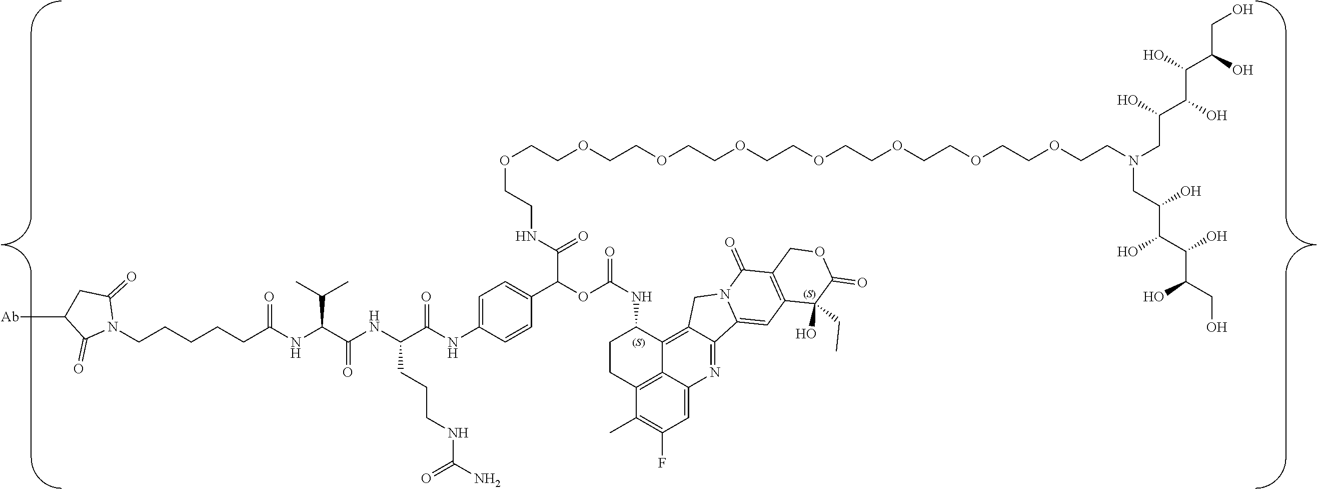

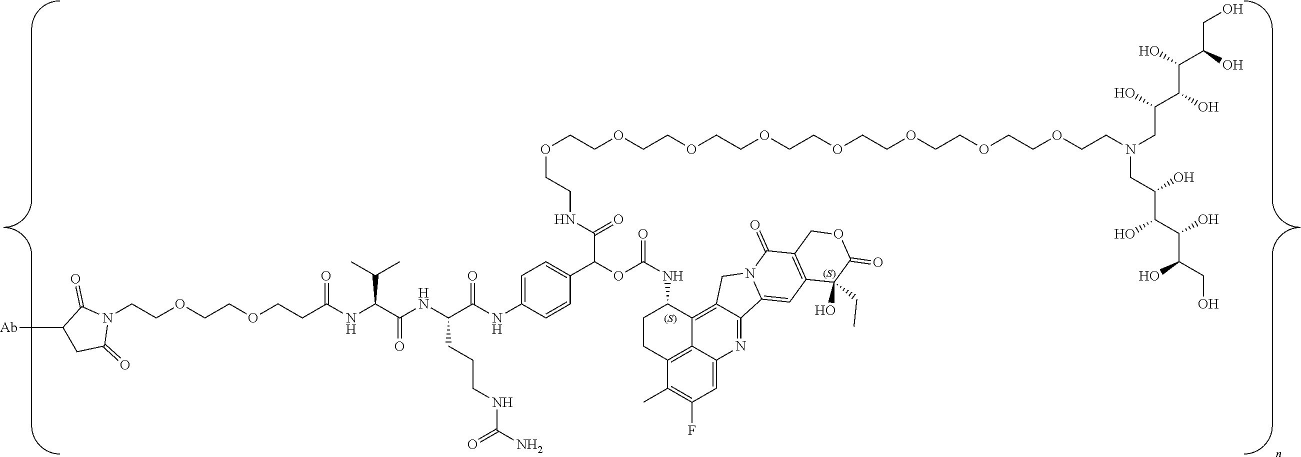

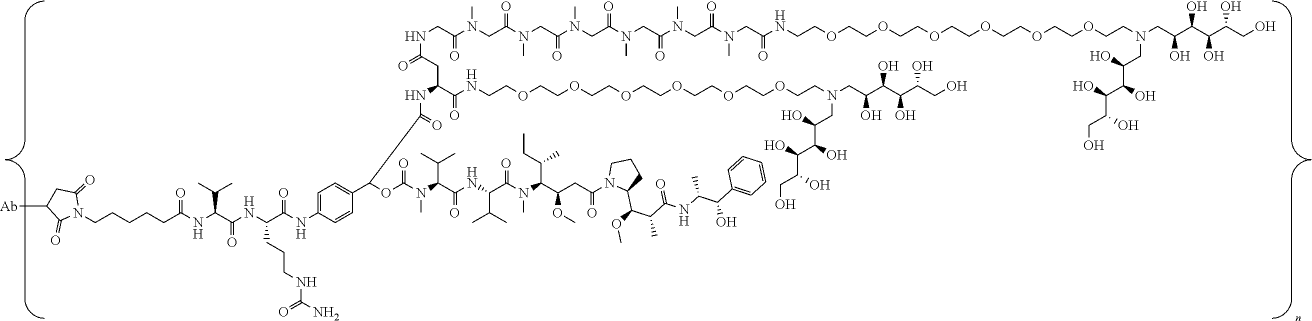

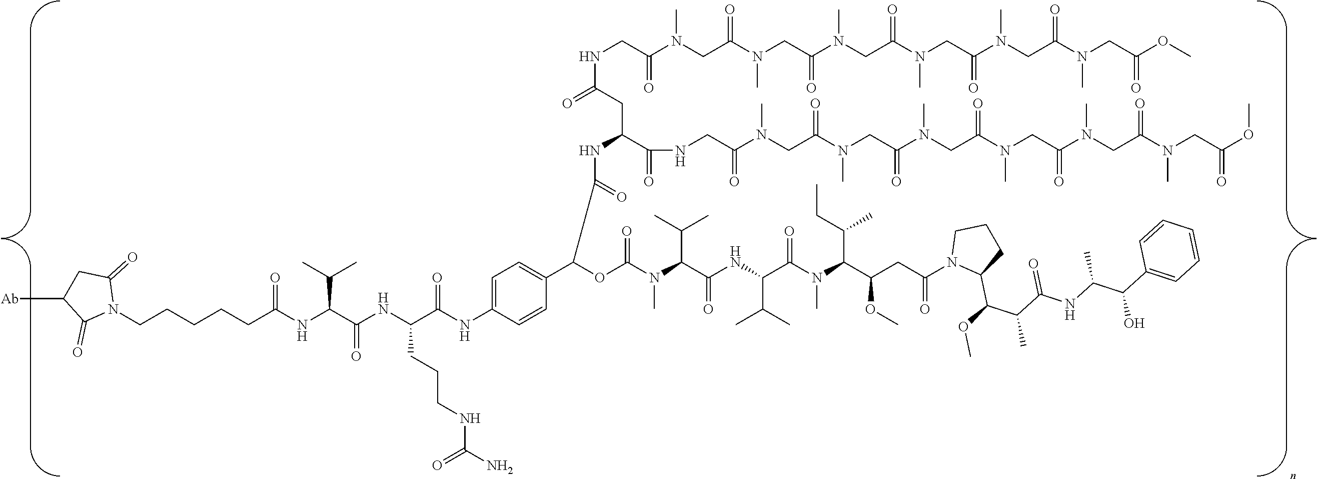

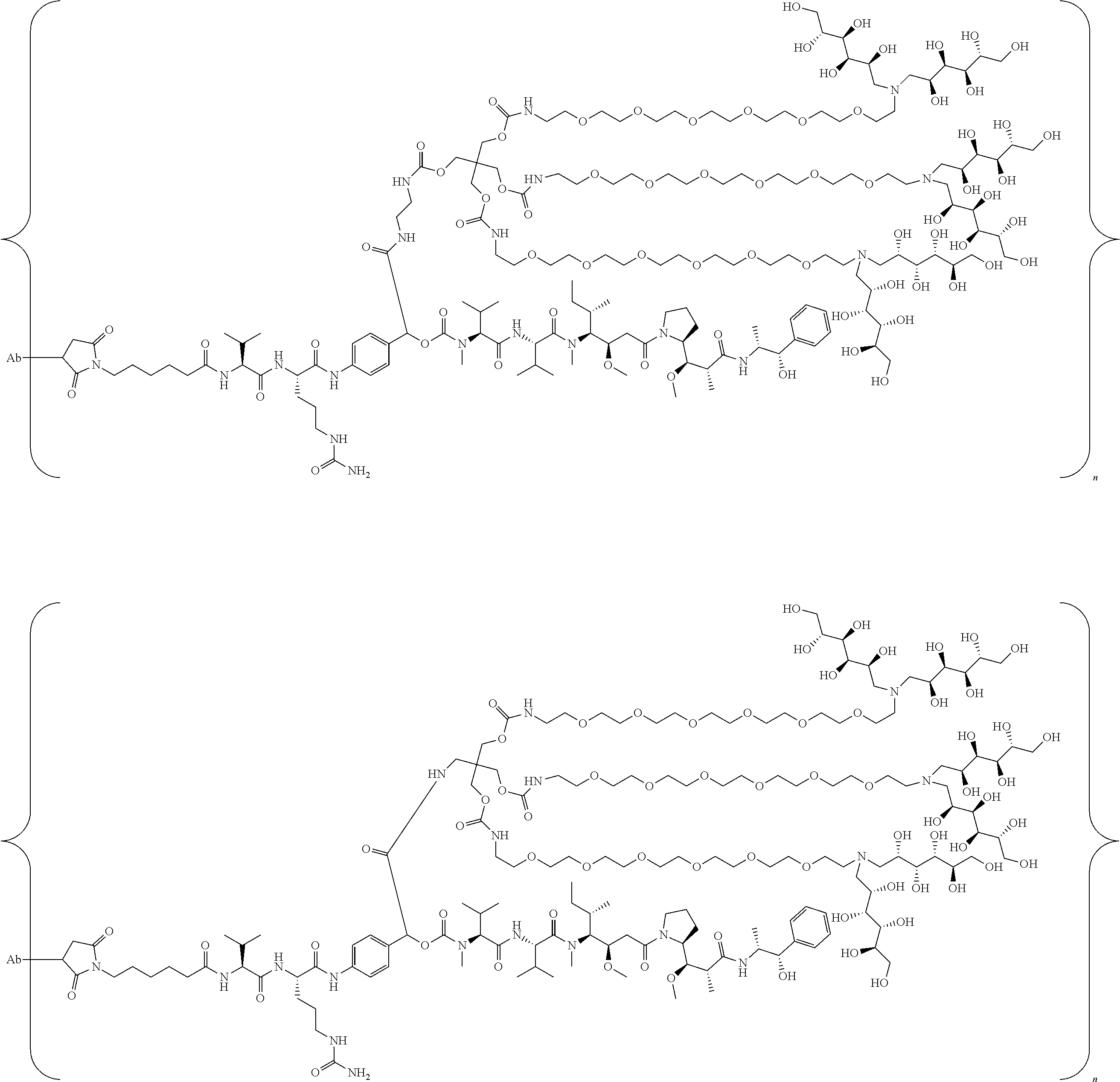

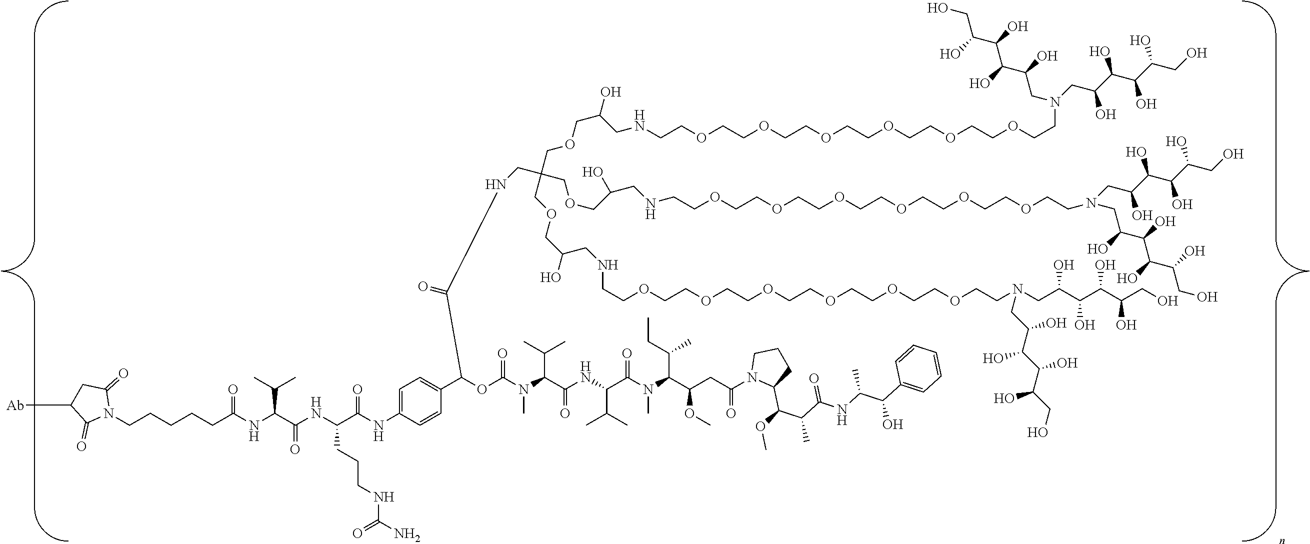

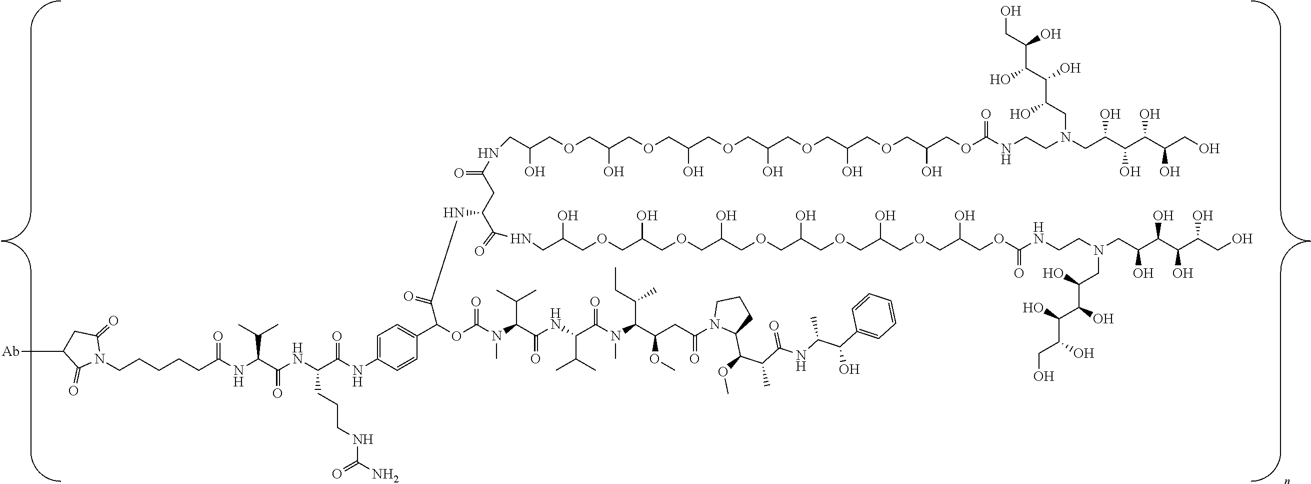

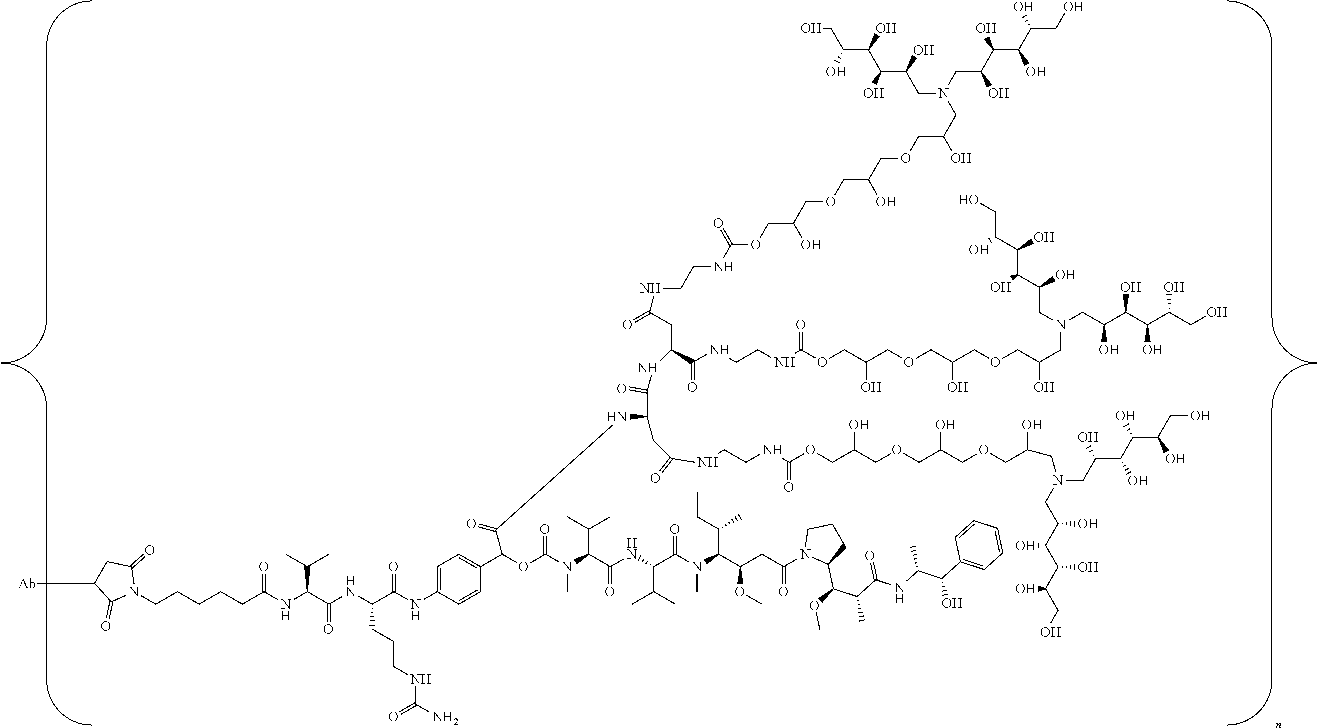

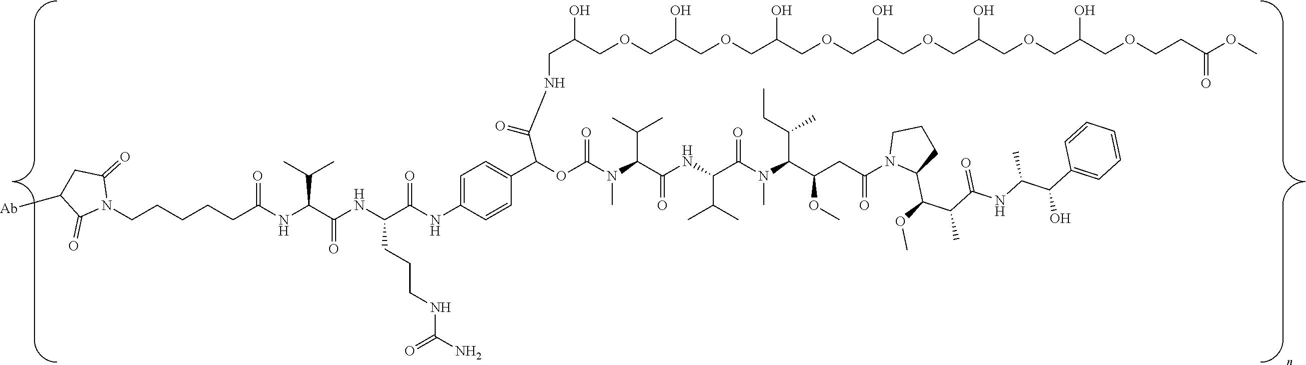

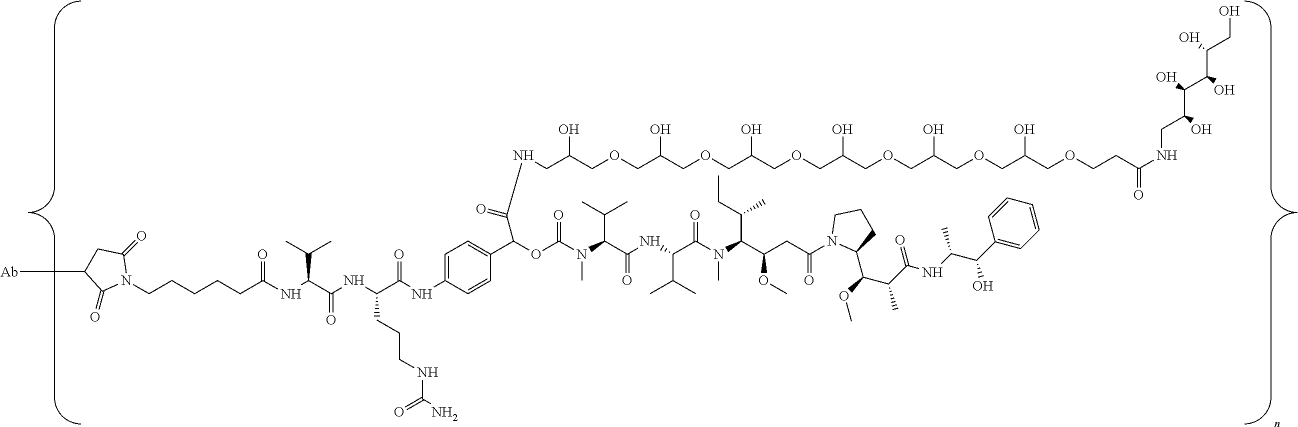

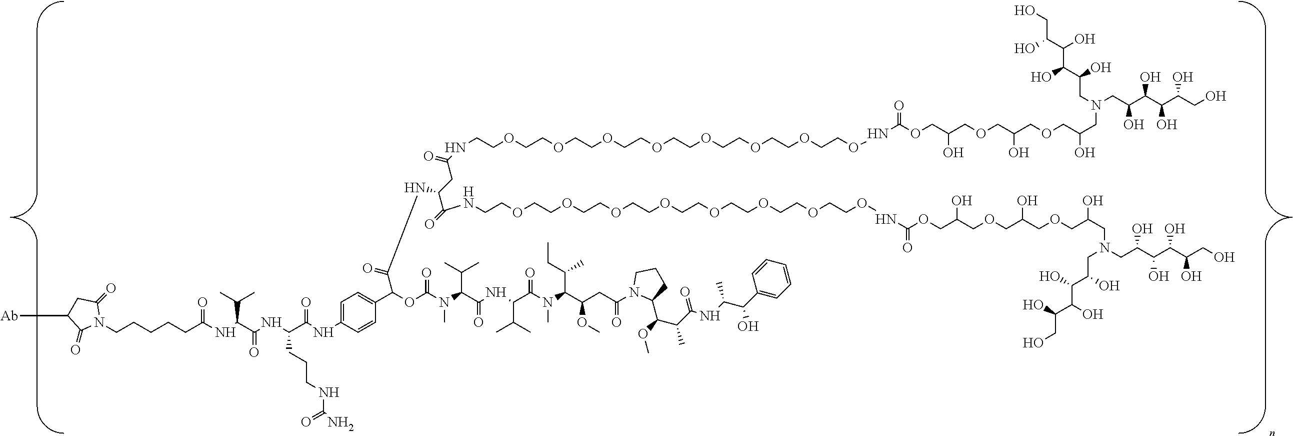

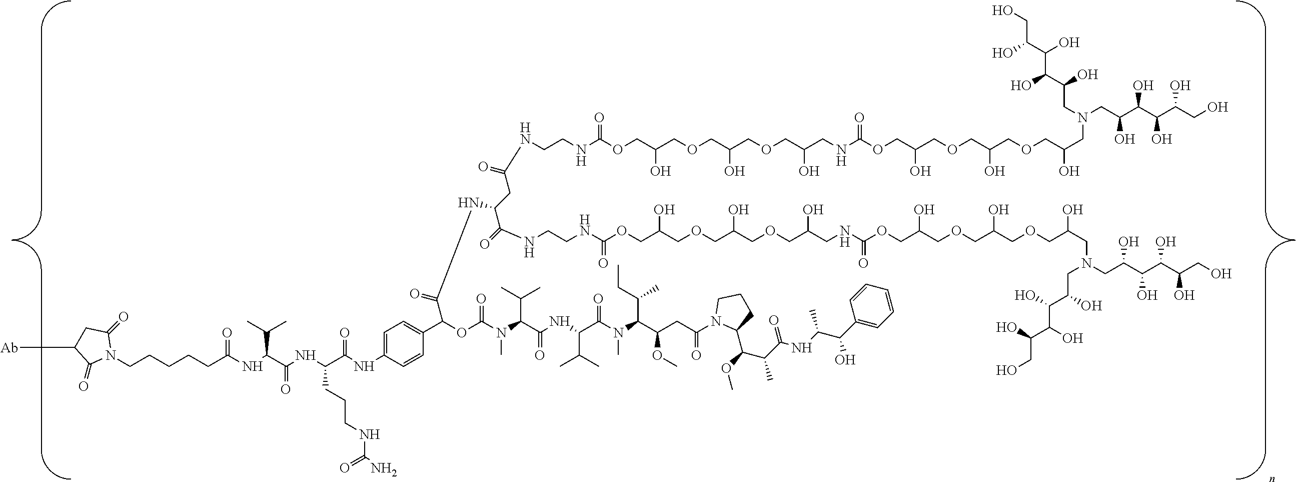

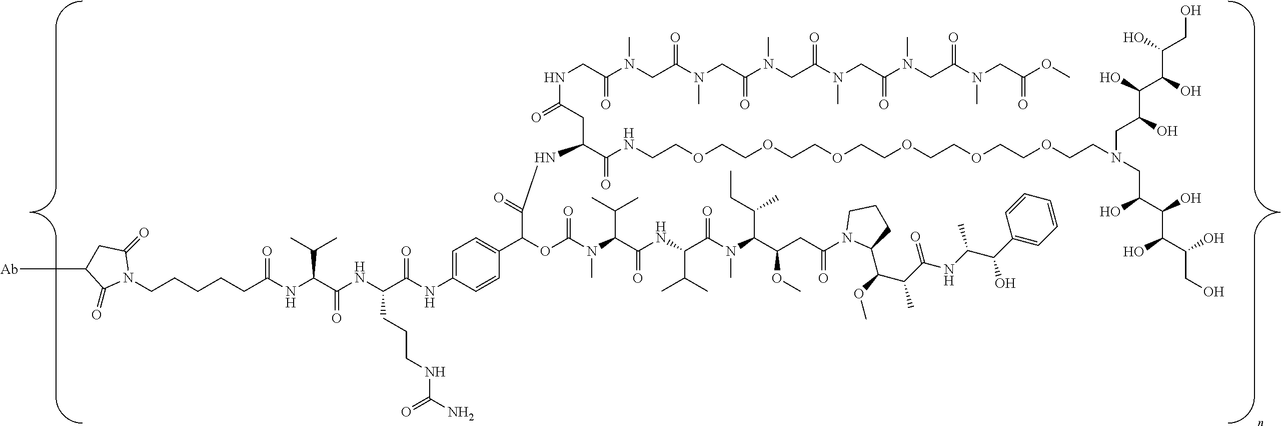

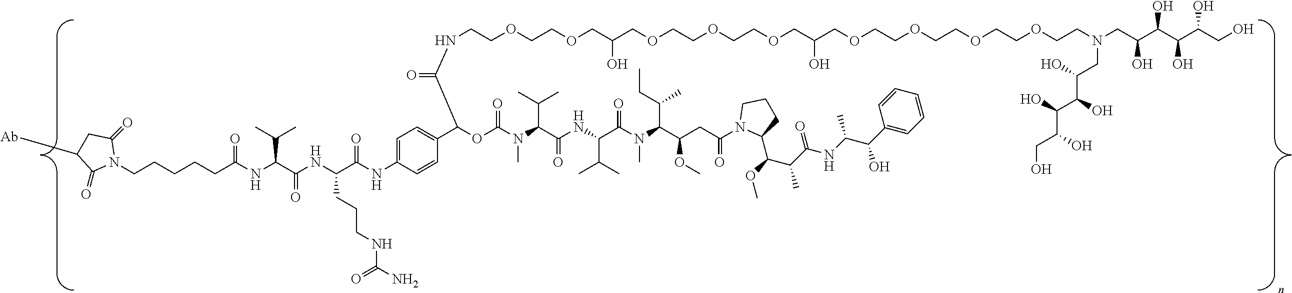

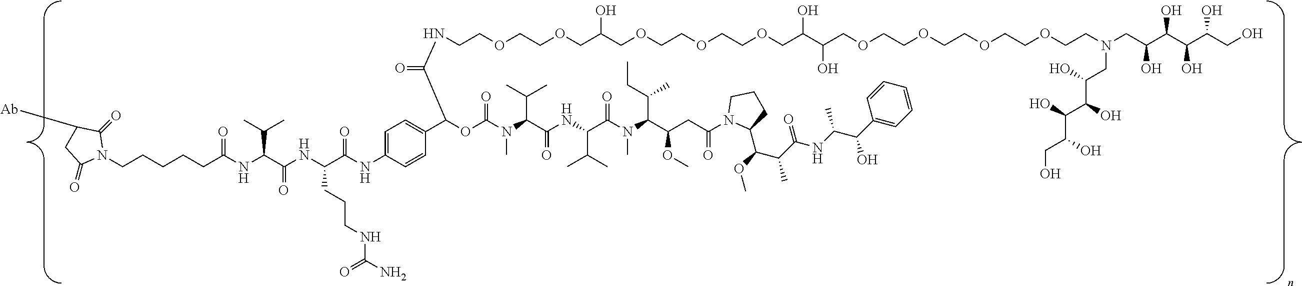

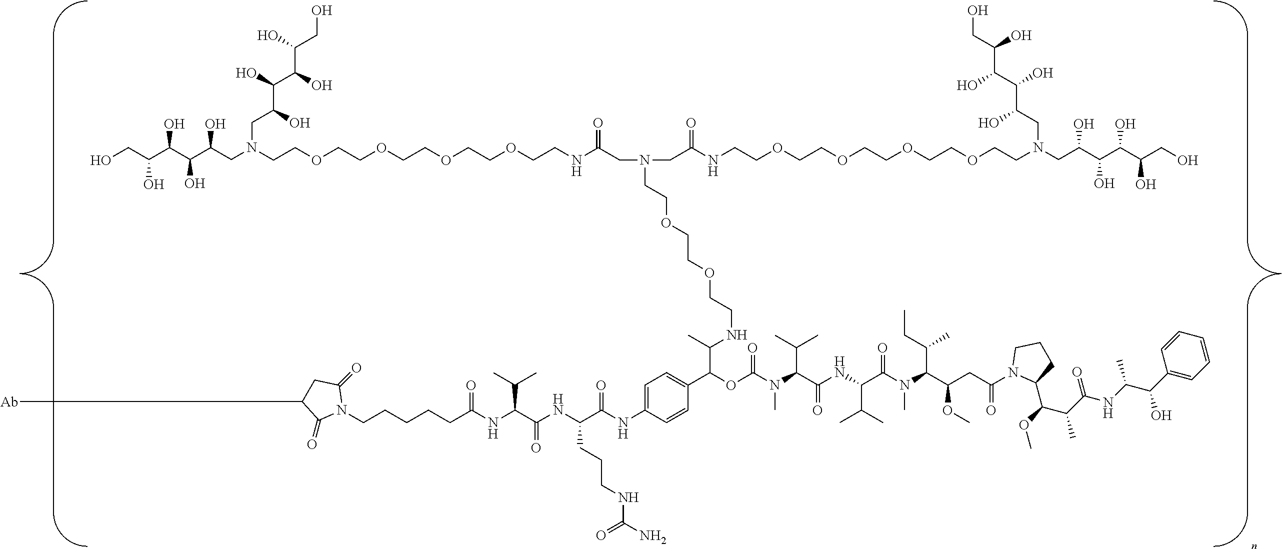

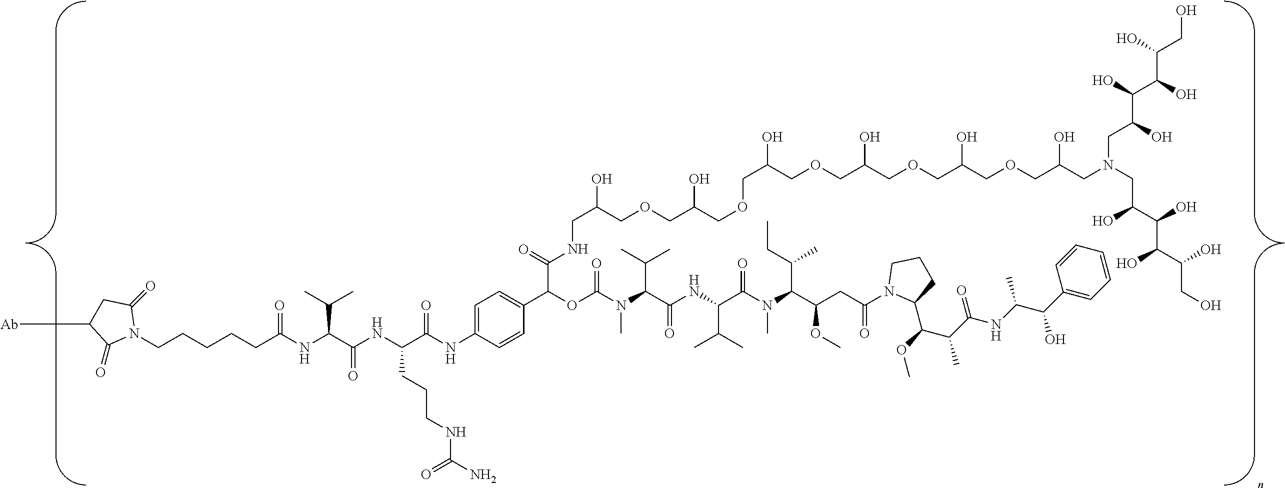

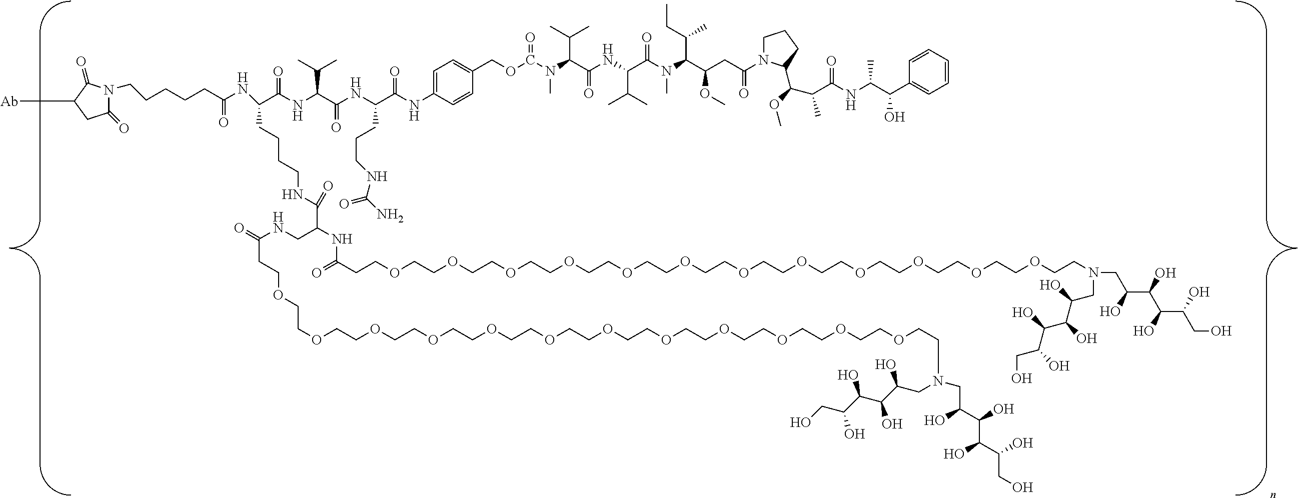

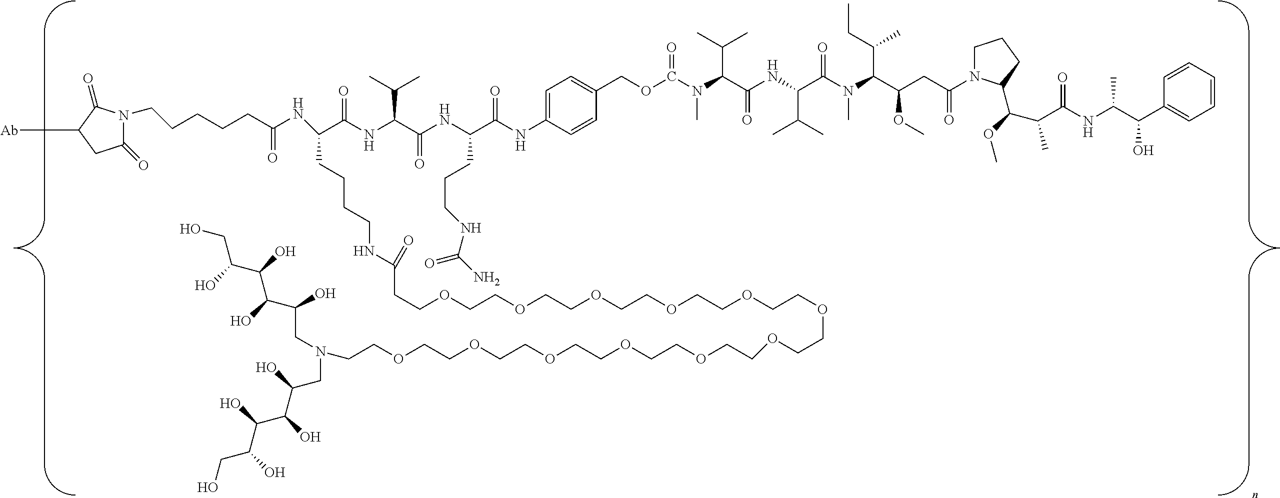

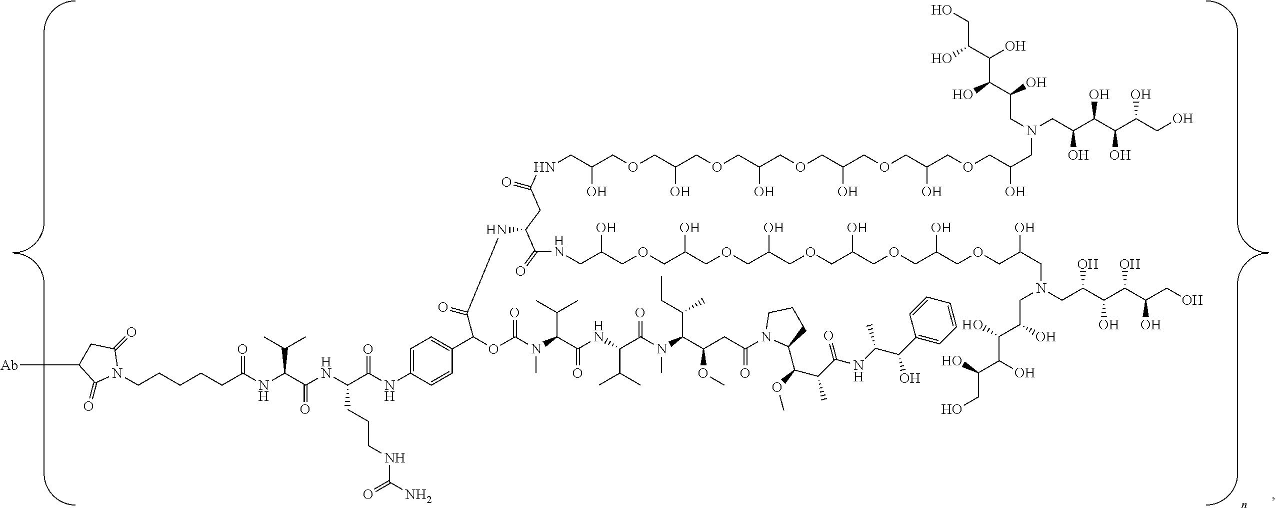

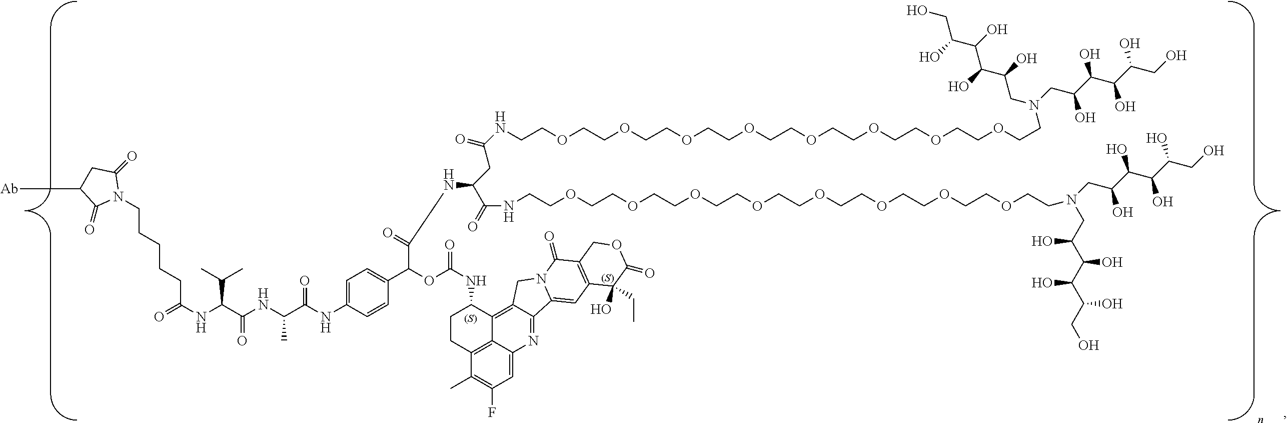

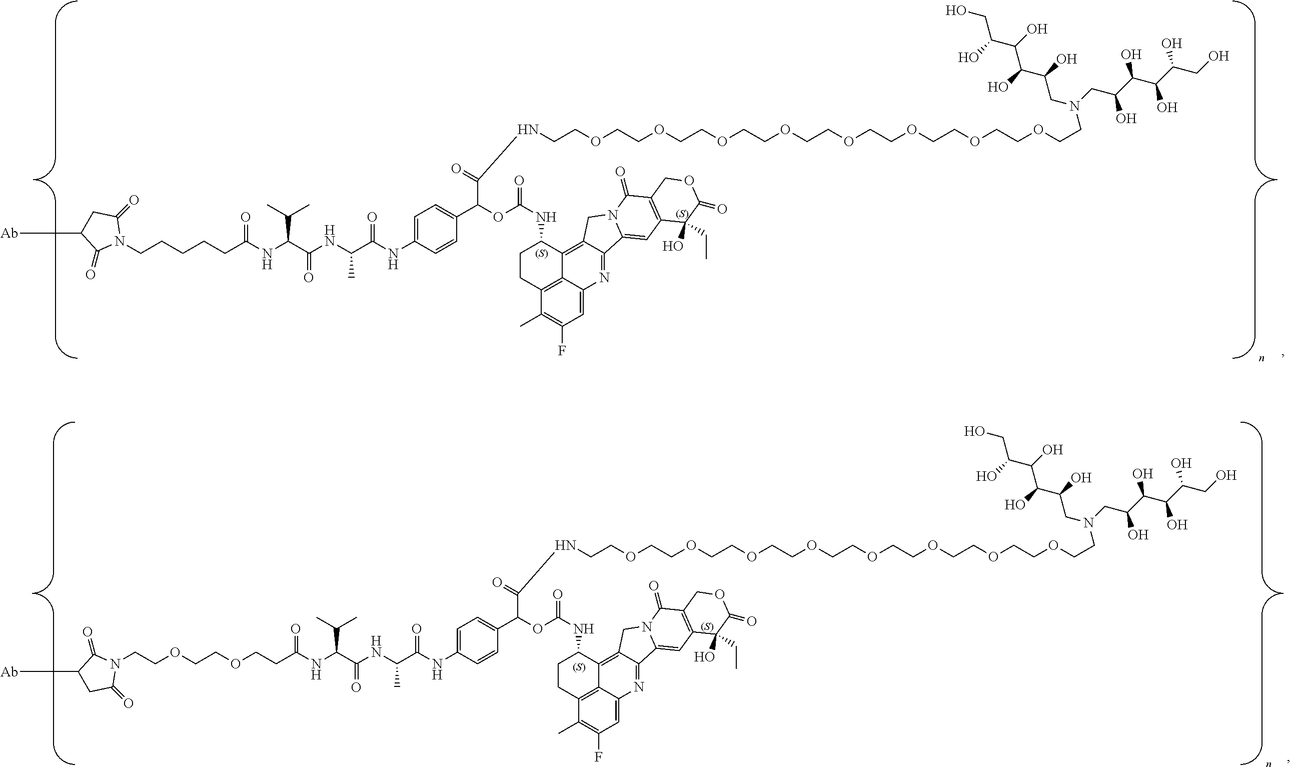

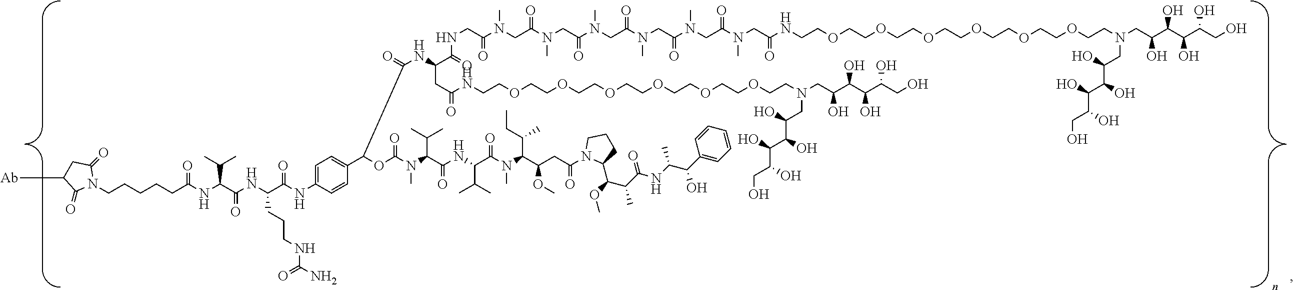

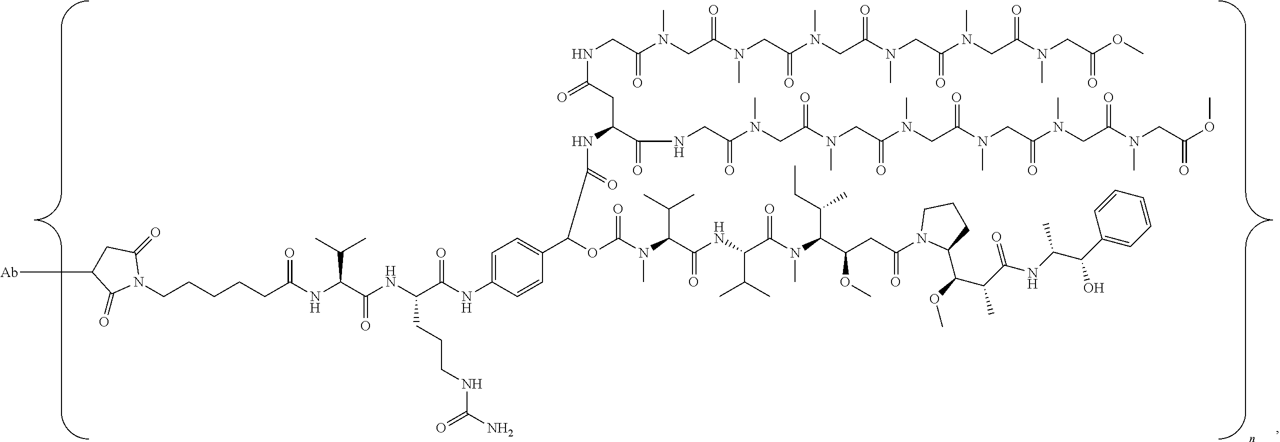

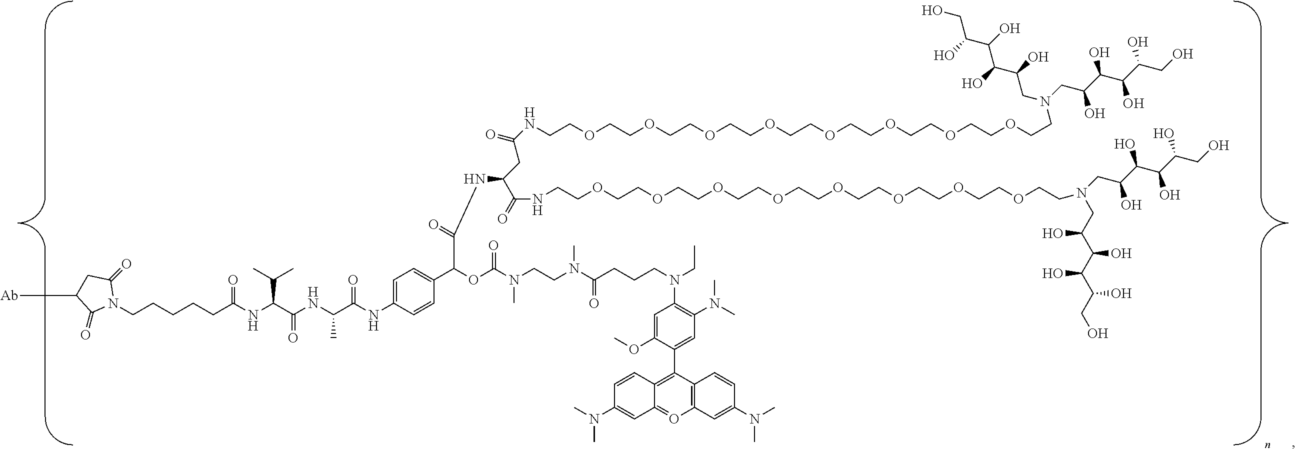

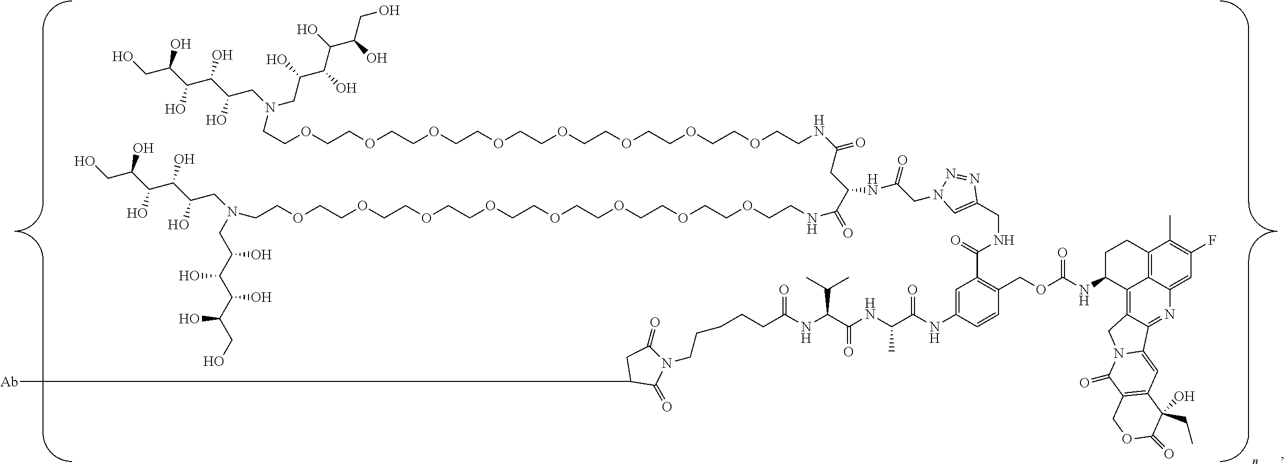

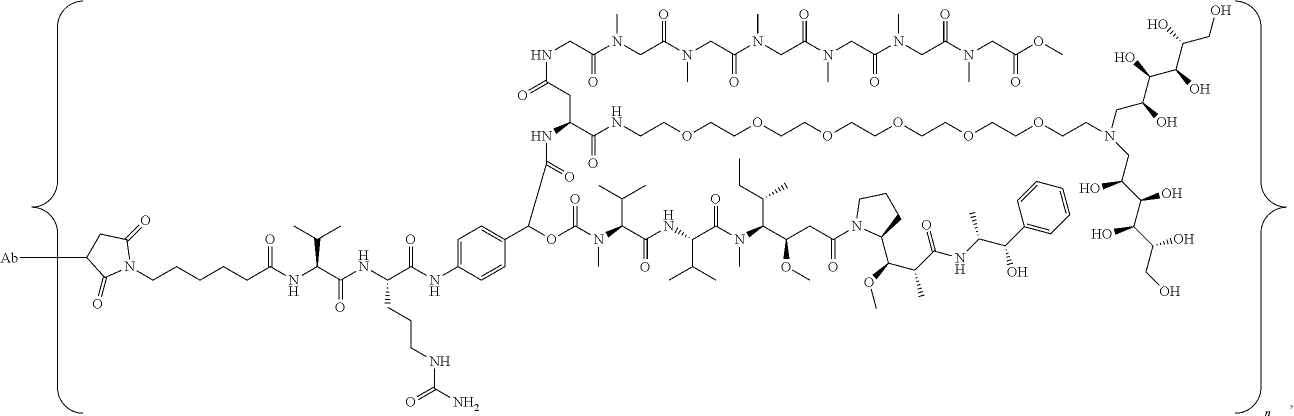

or a stereoisomer thereof, wherein Ab is a SLITRK6 binding agent and n is p^{load}.

Embodiment 140. The conjugate of any one of Embodiments 137-138, selected from the following:

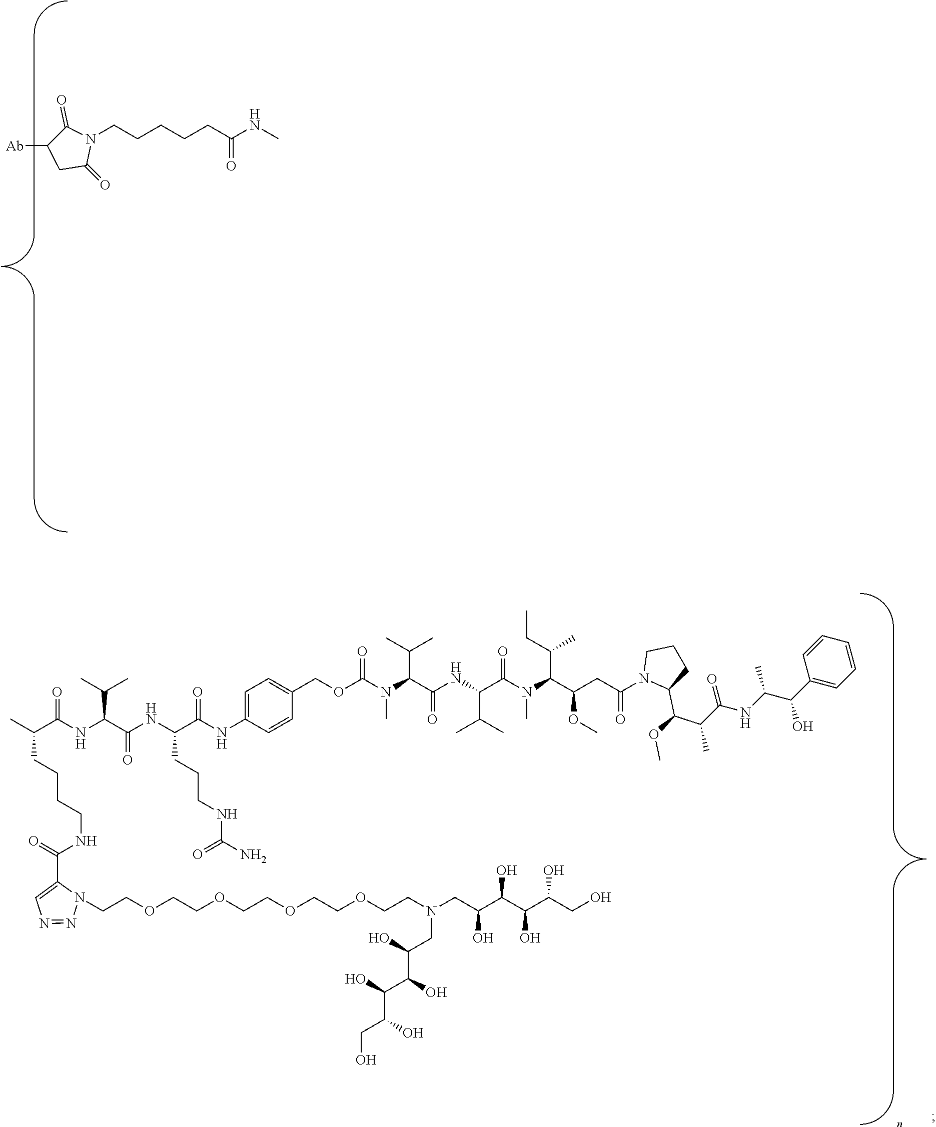

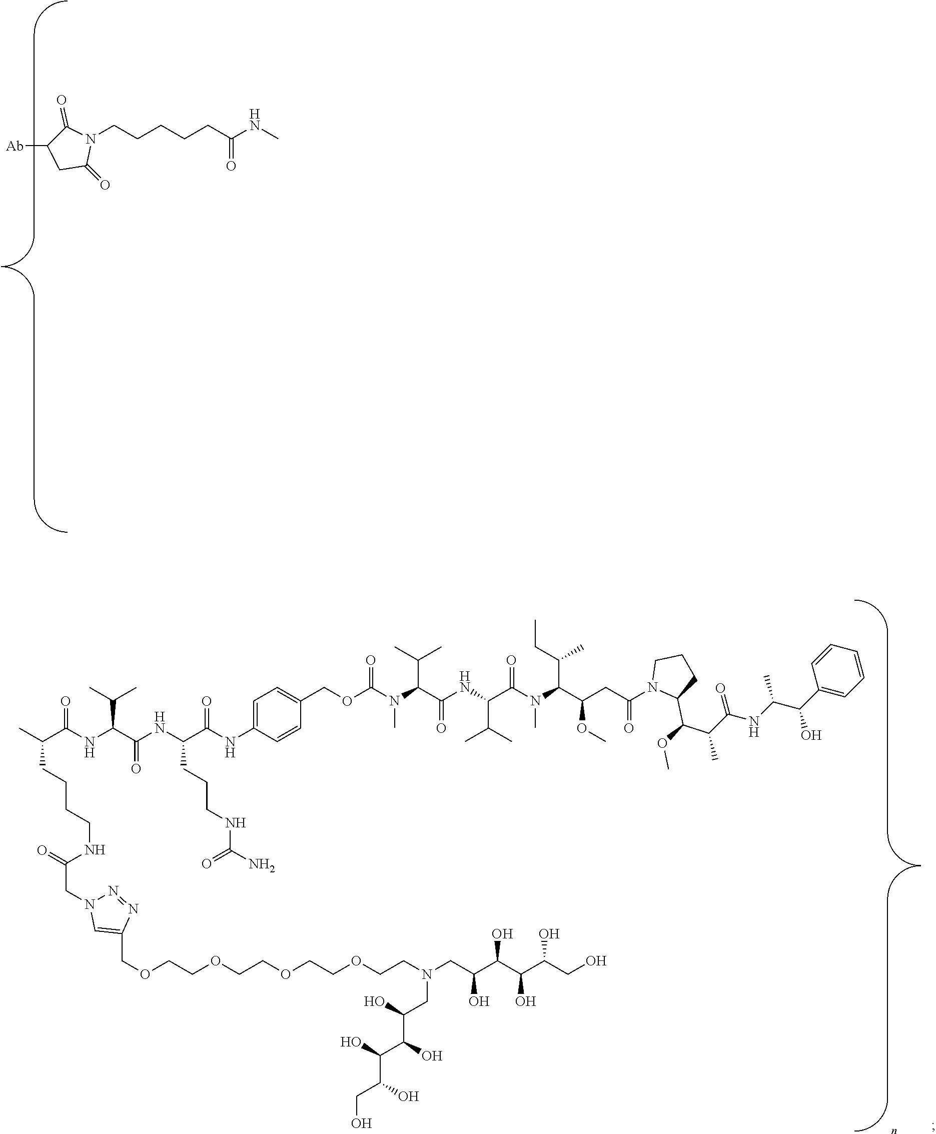

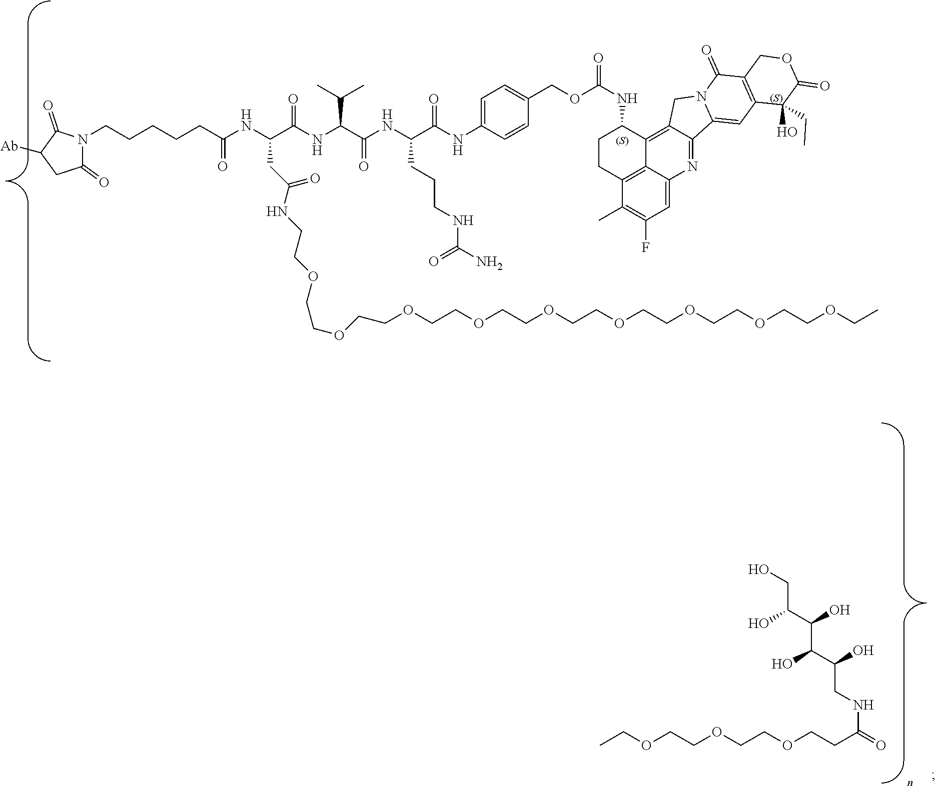

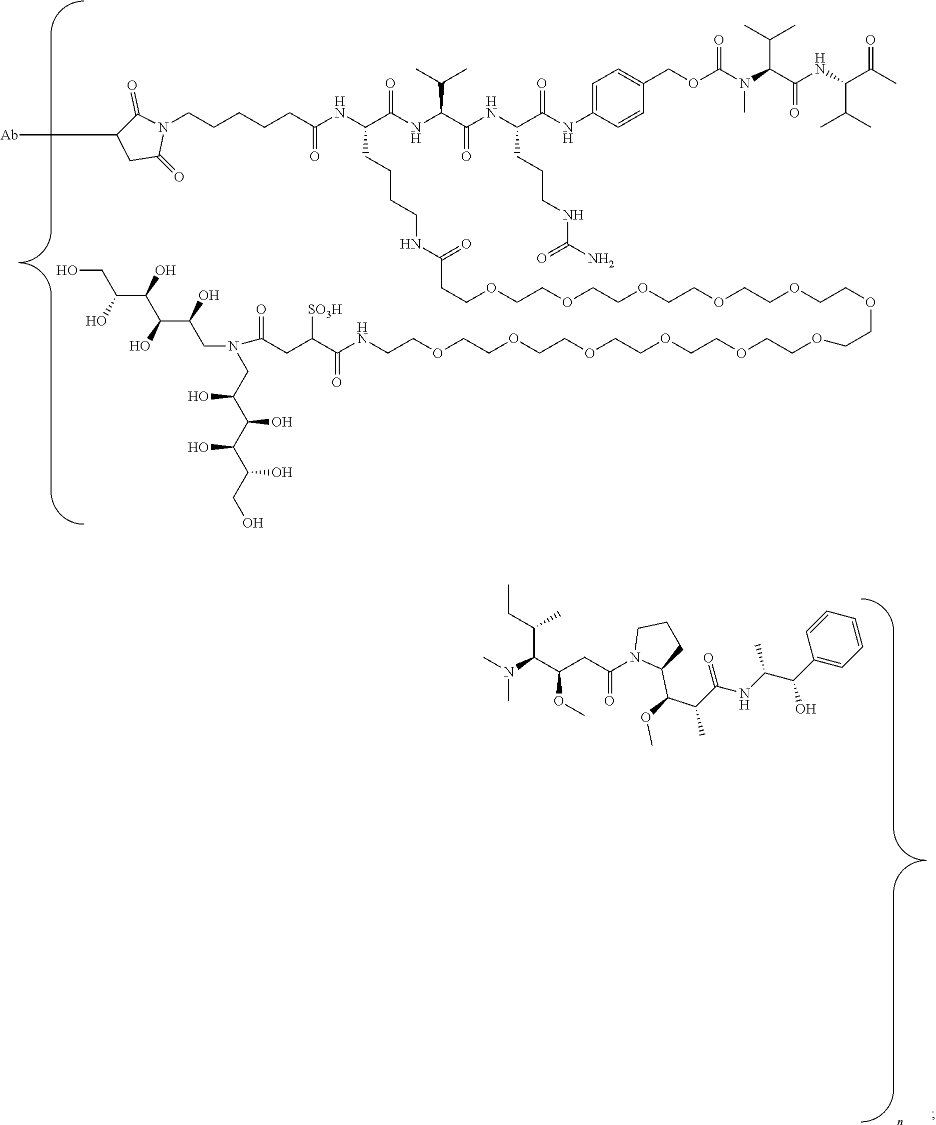

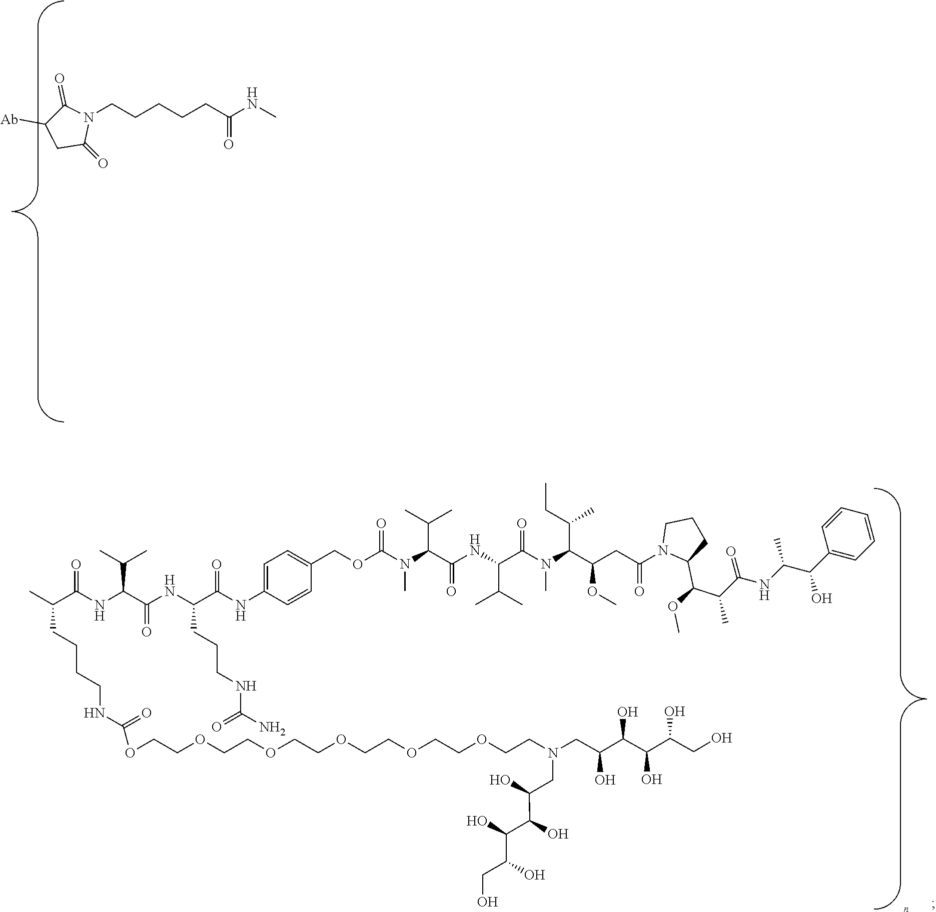

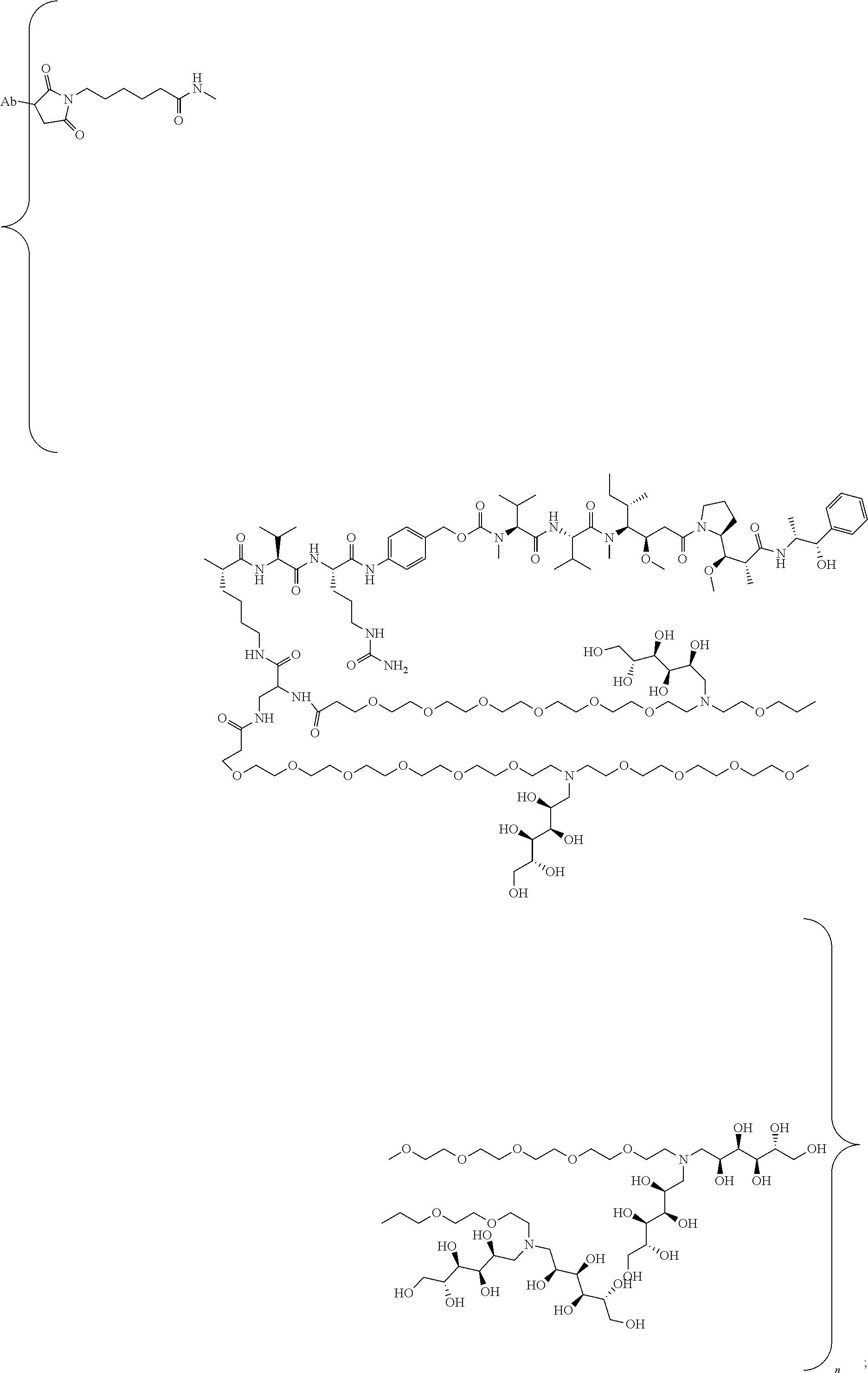

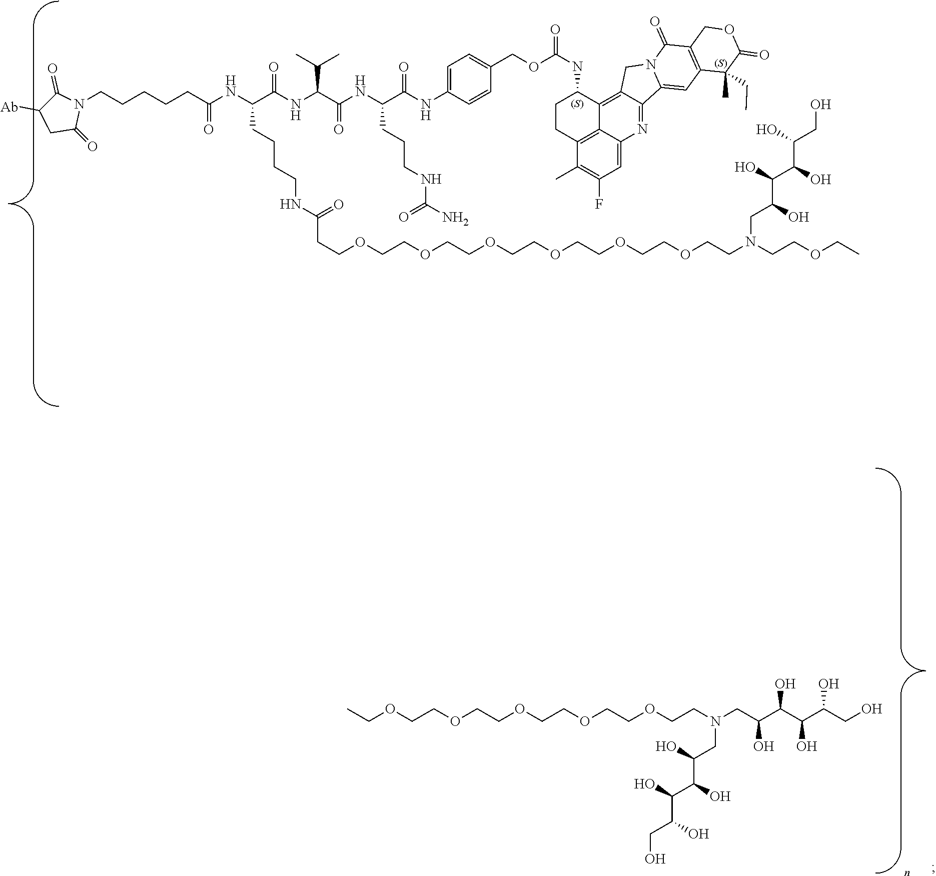

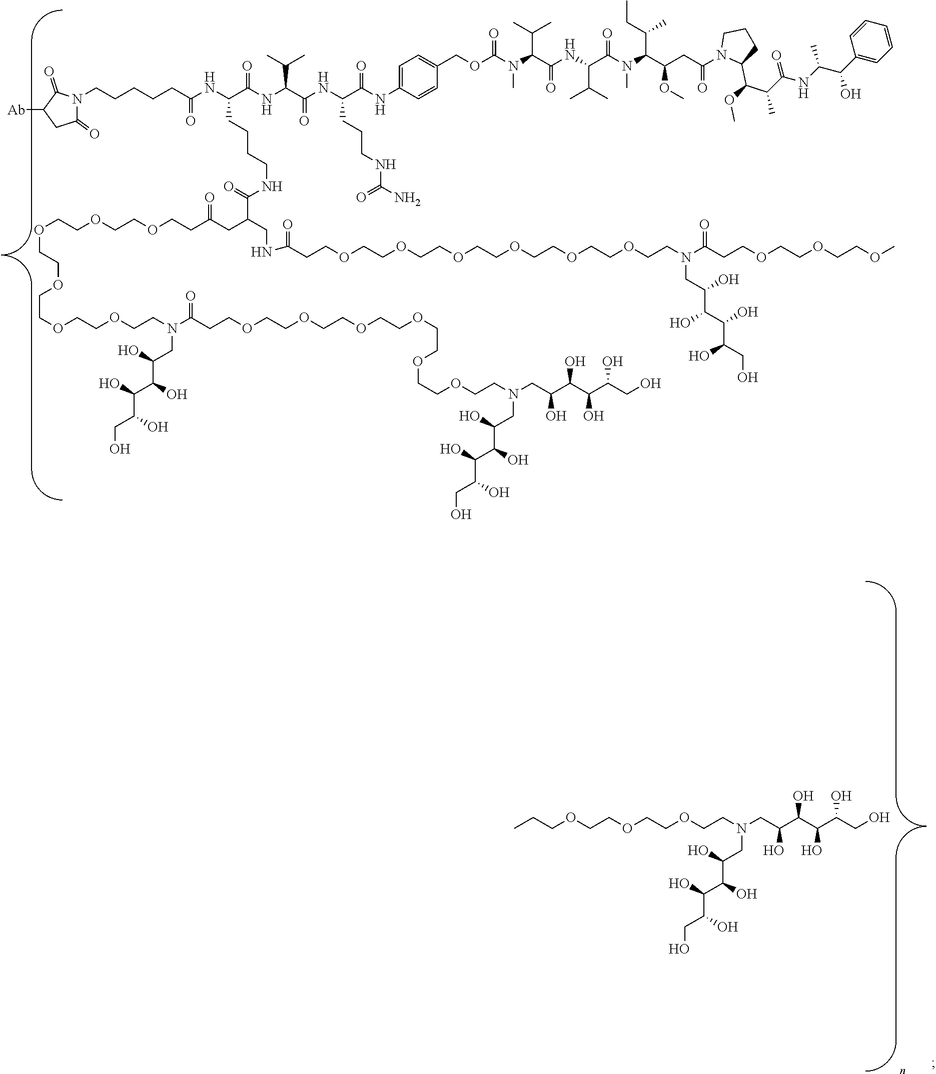

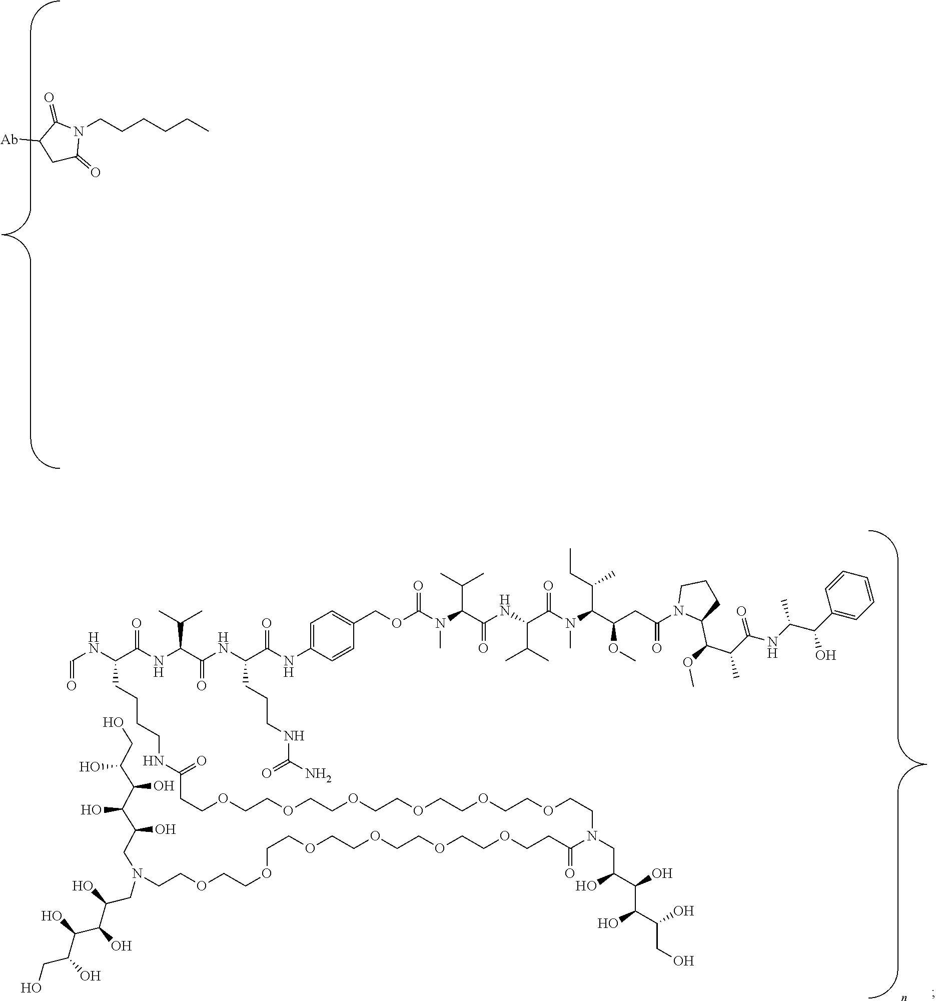

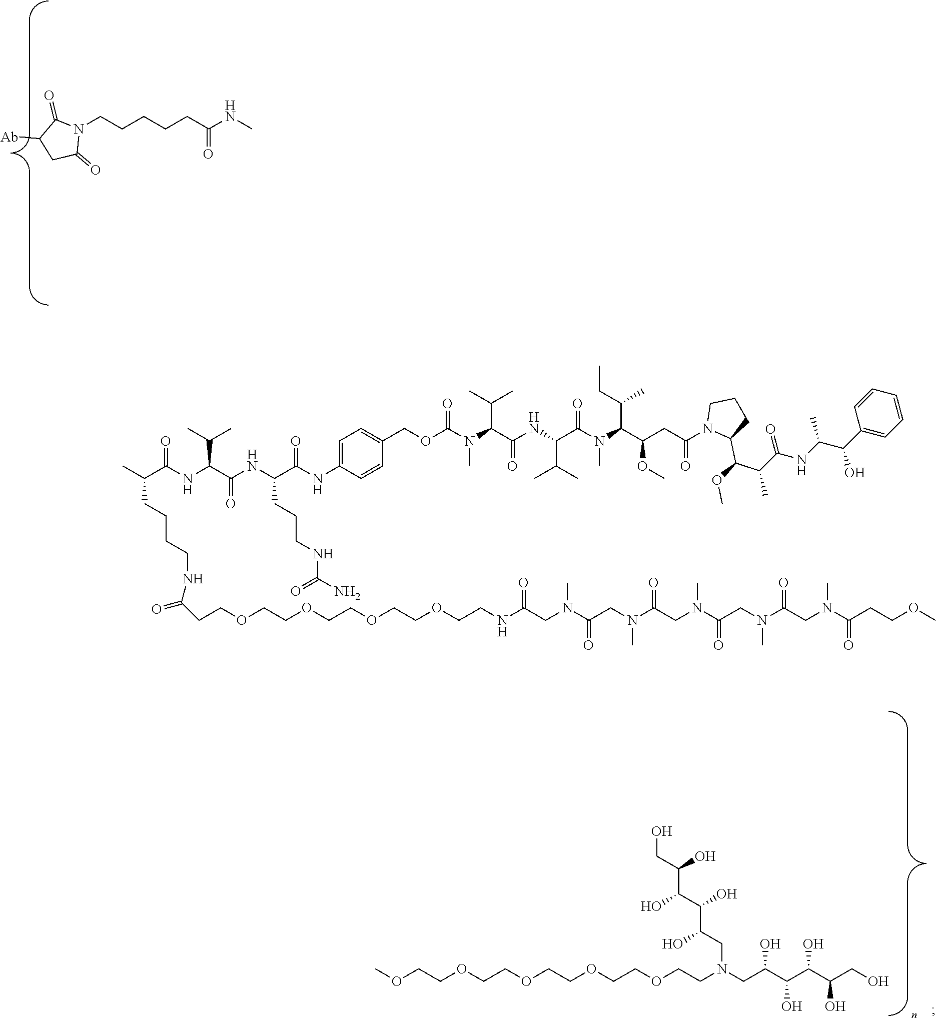

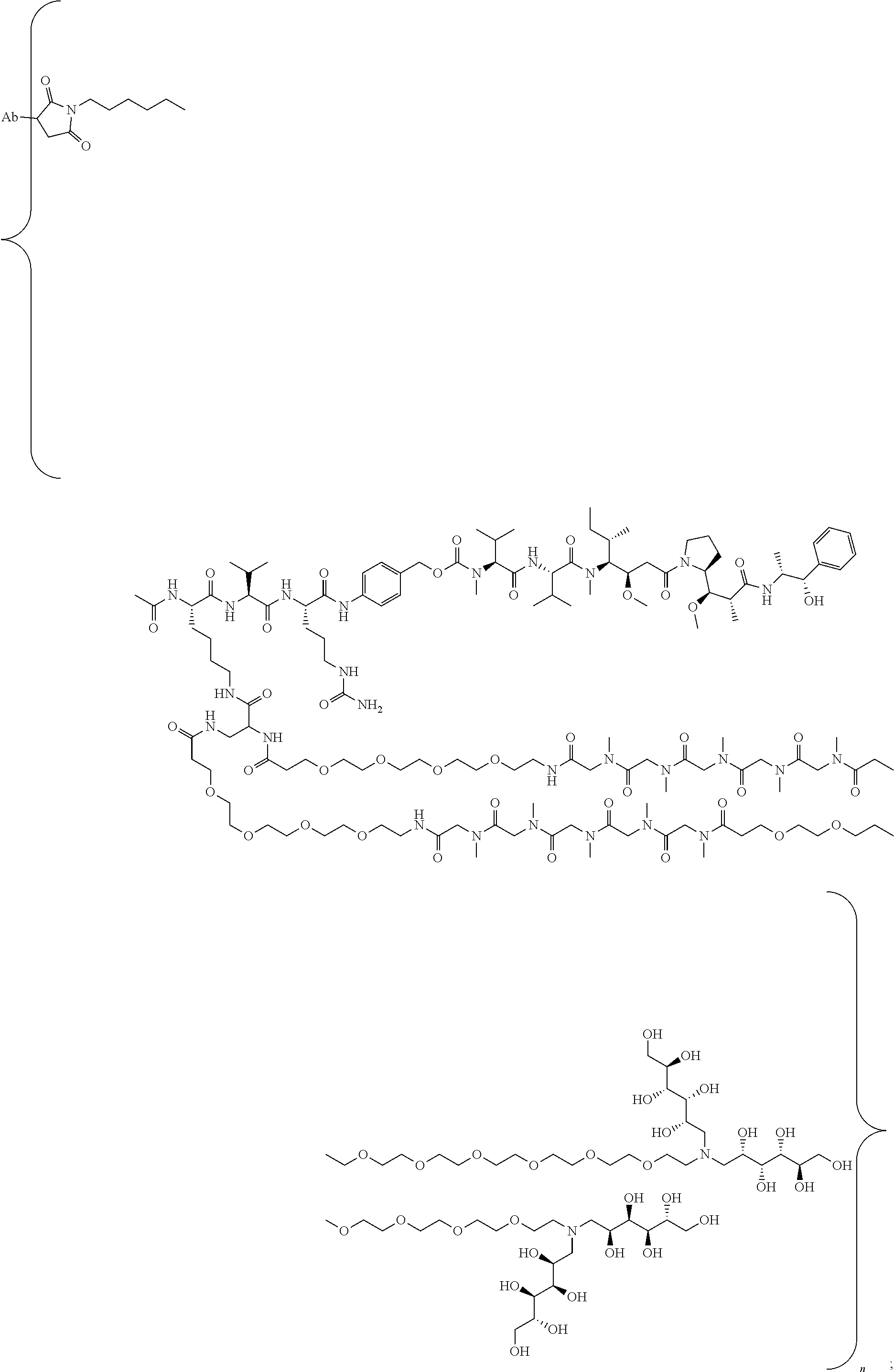

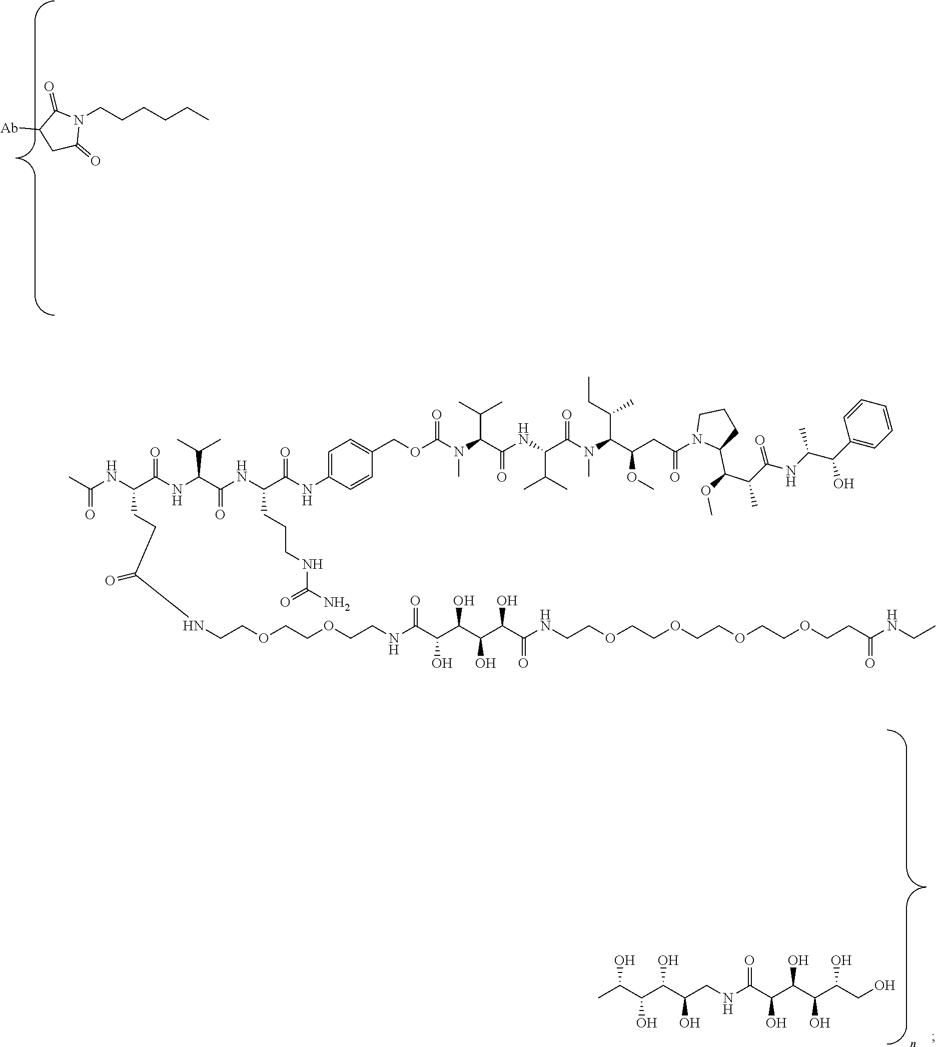

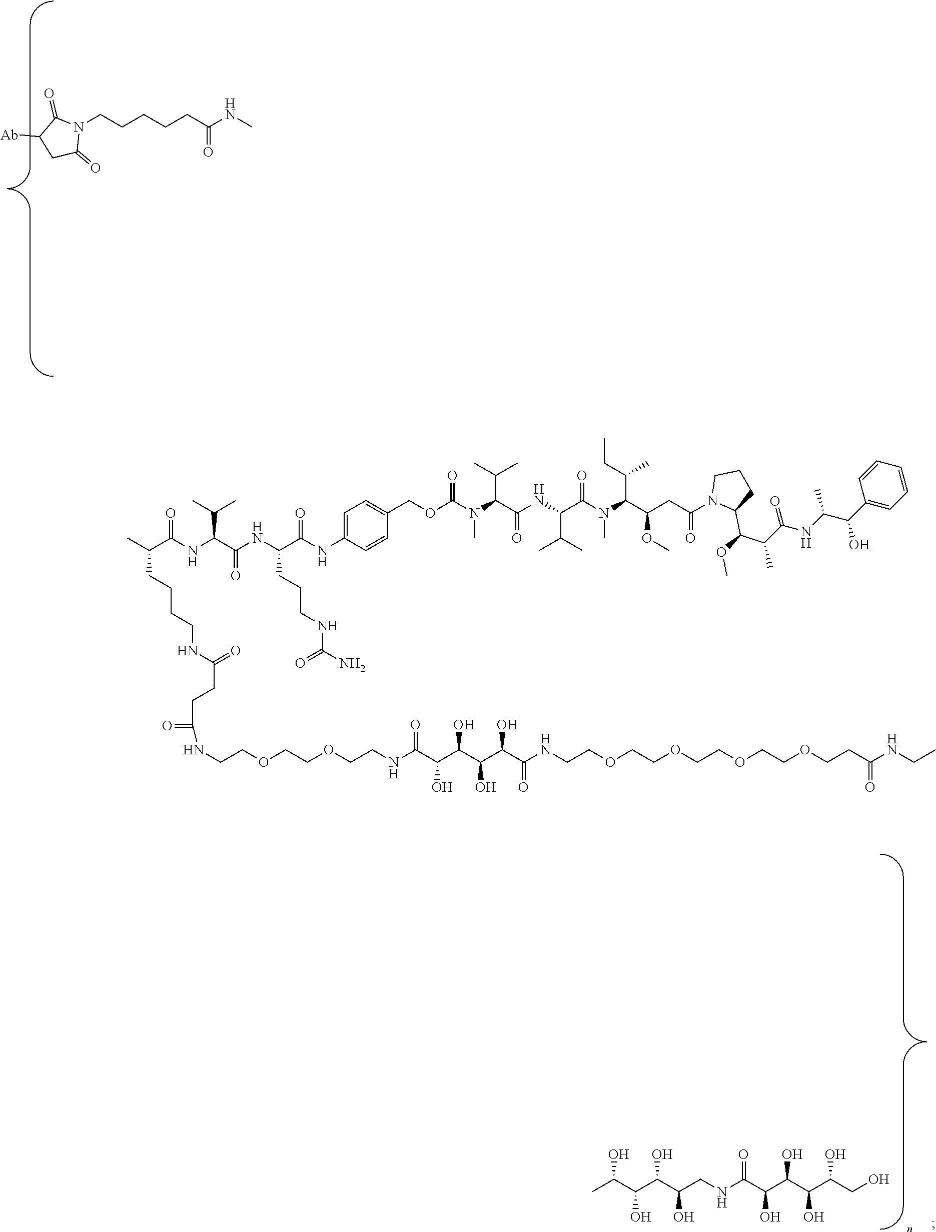

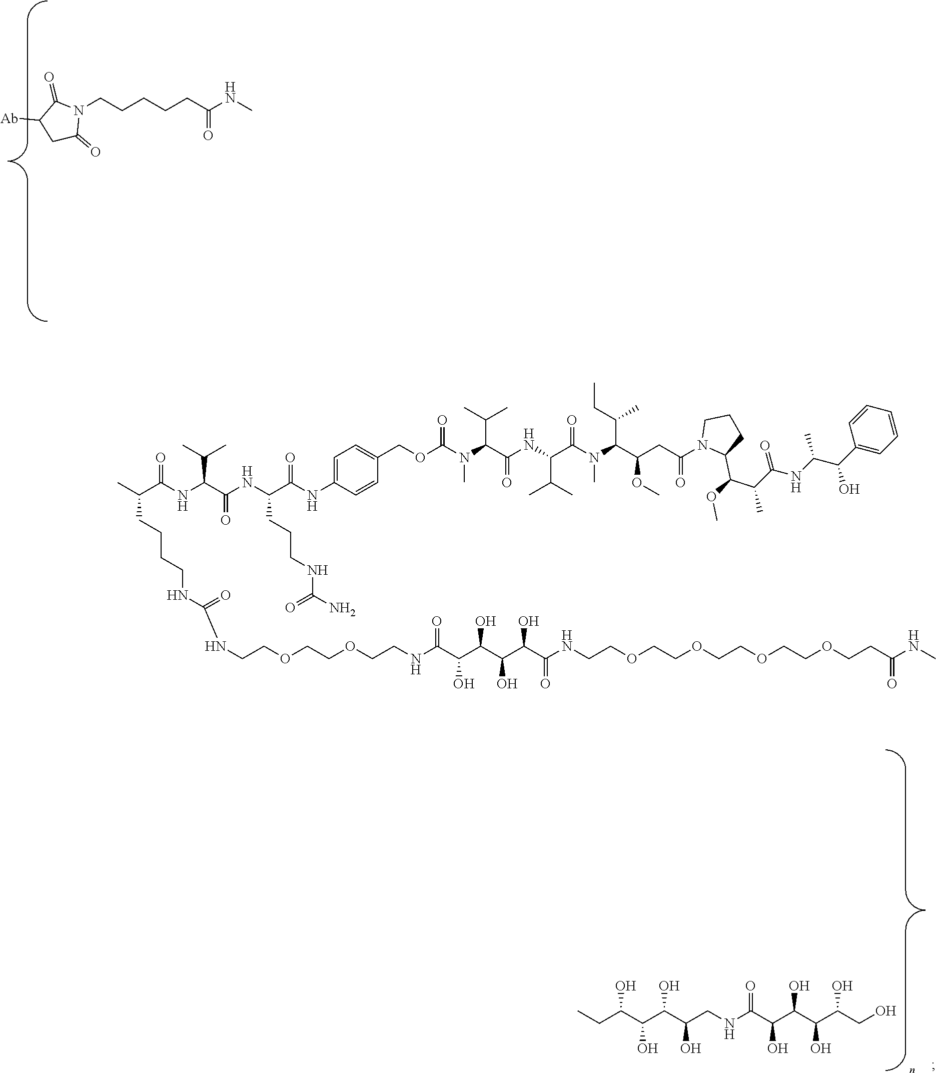

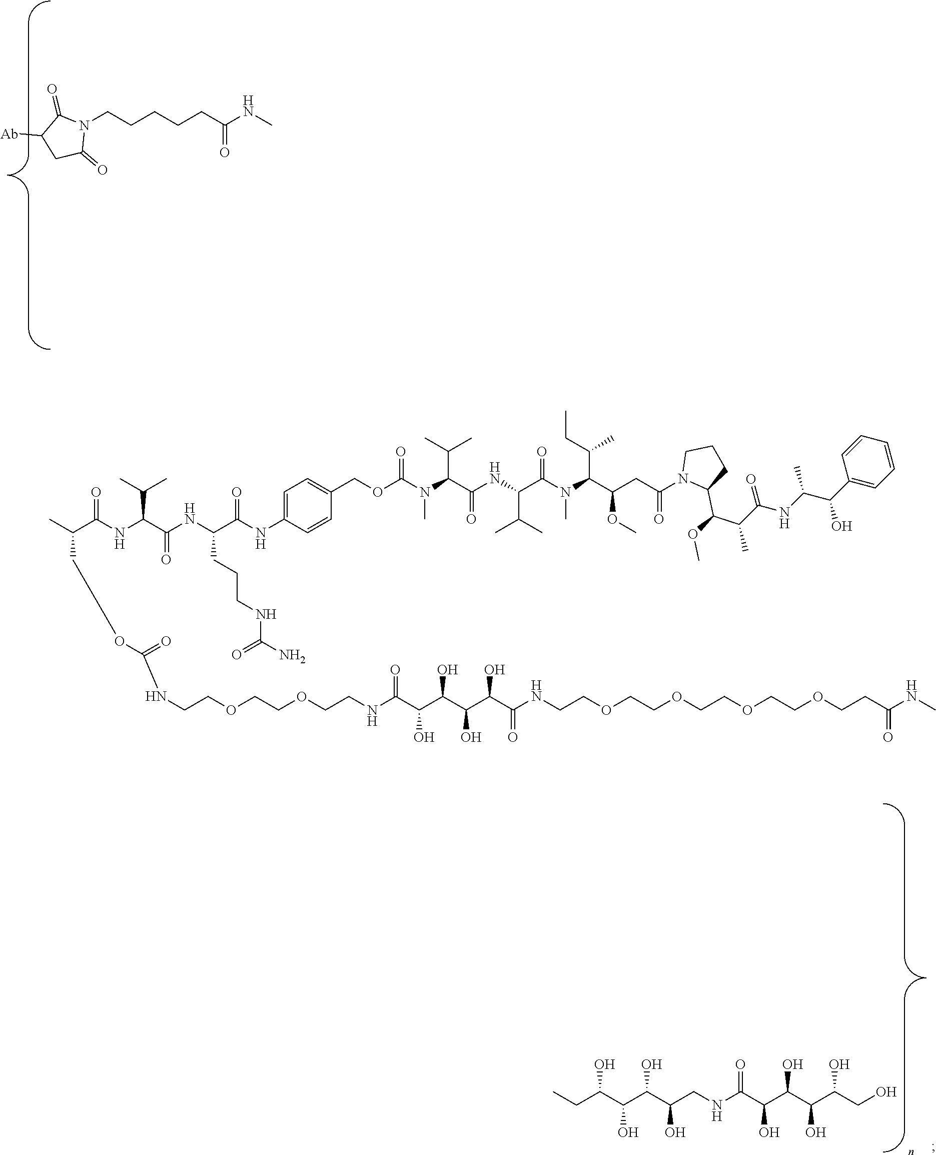

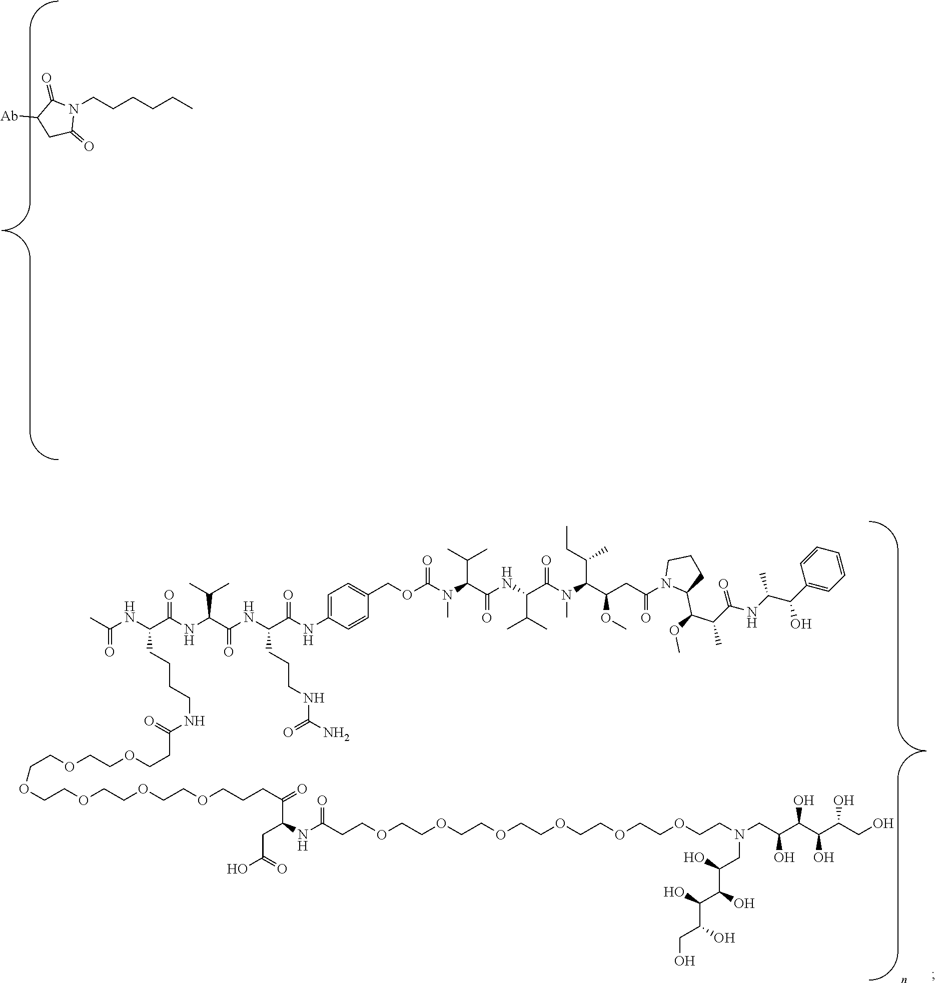

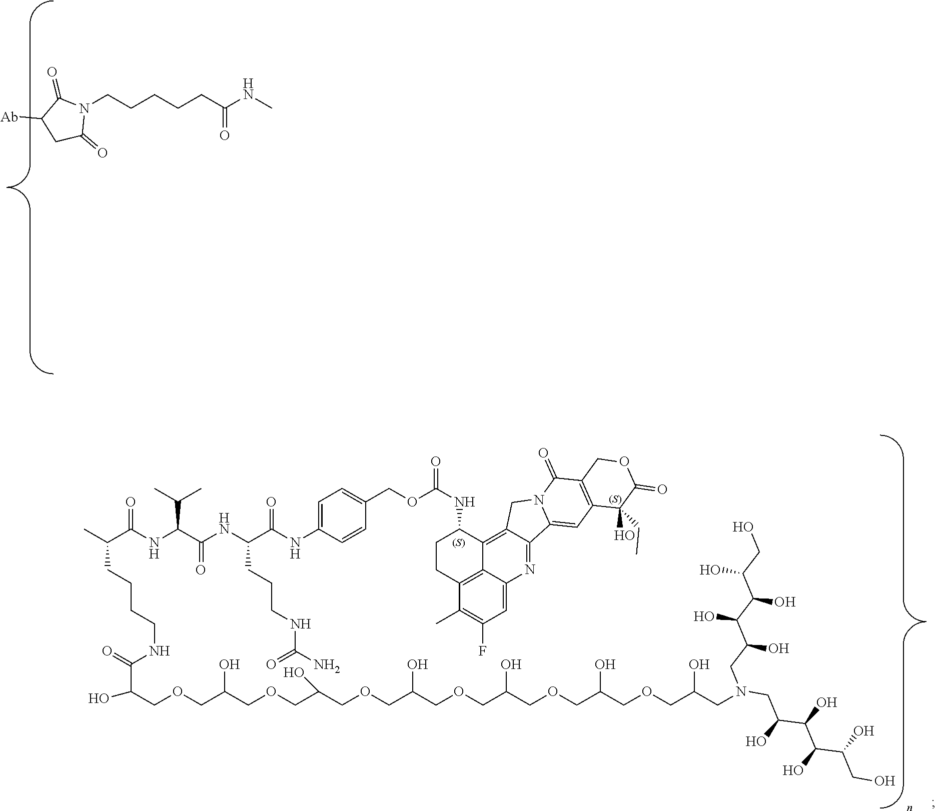

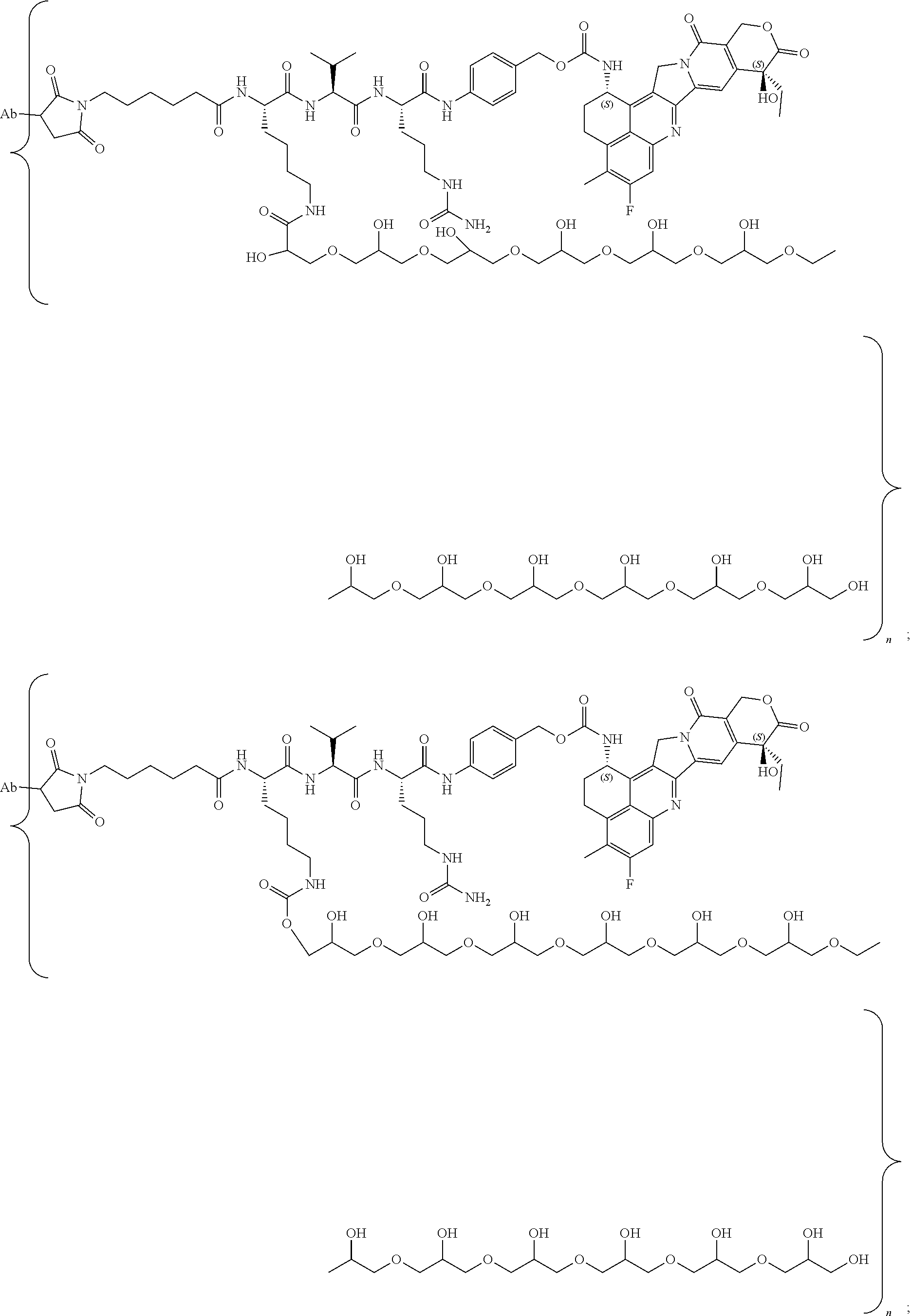

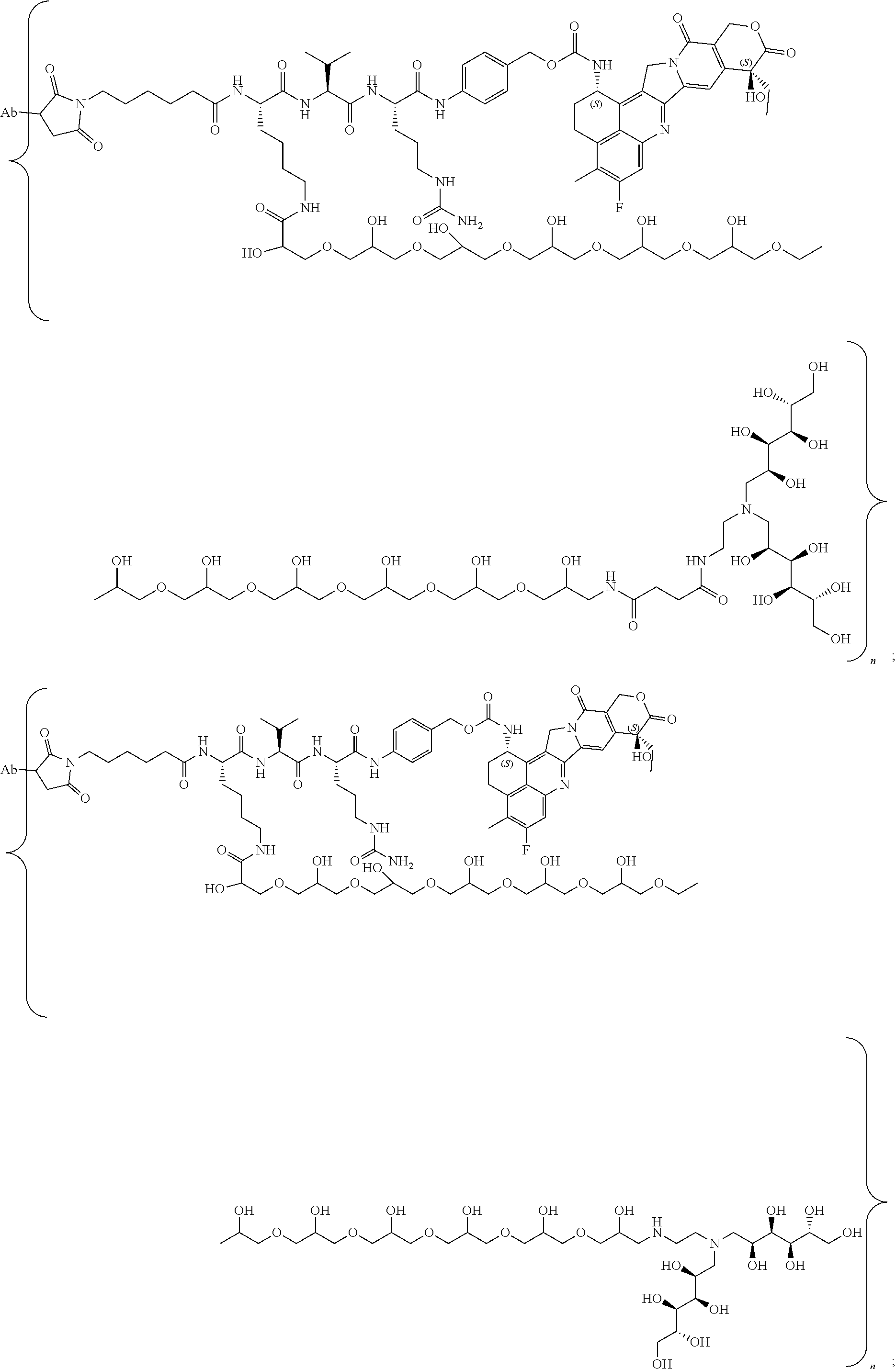

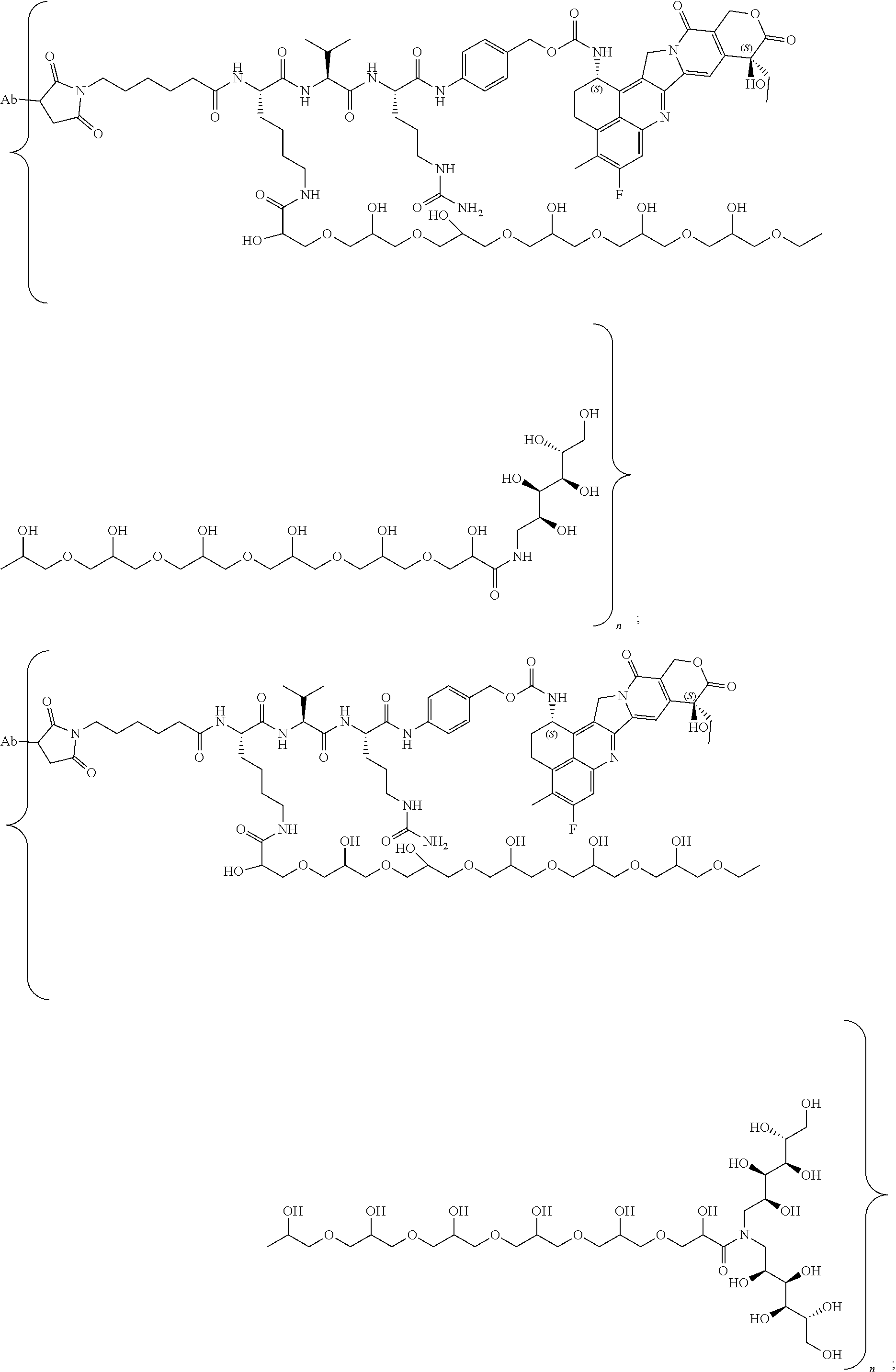

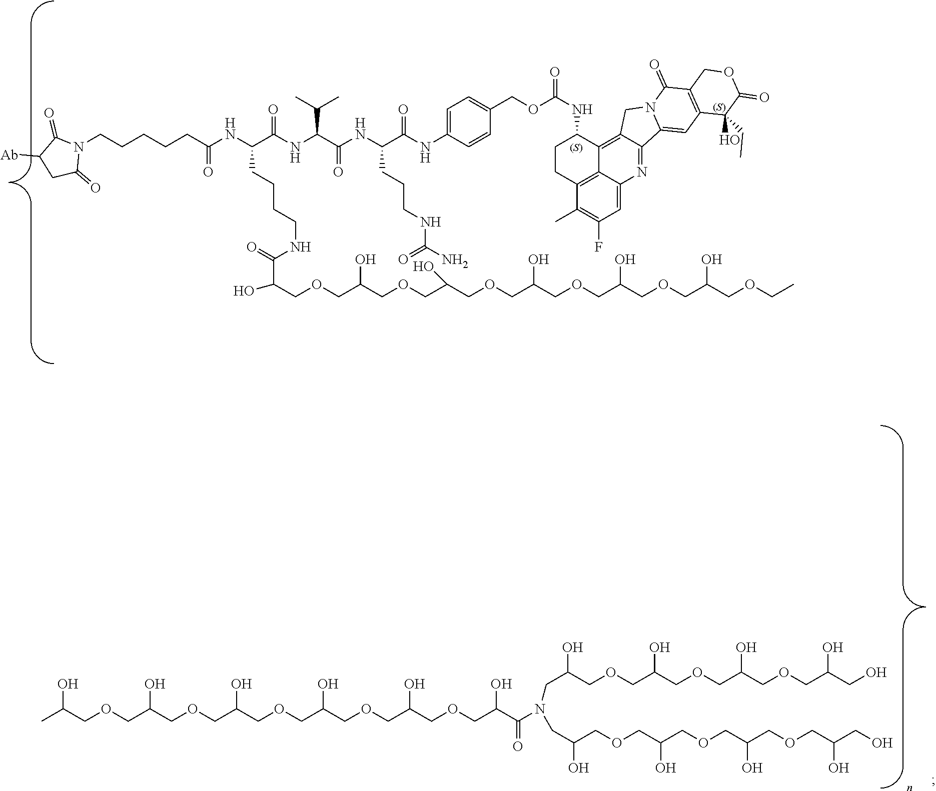

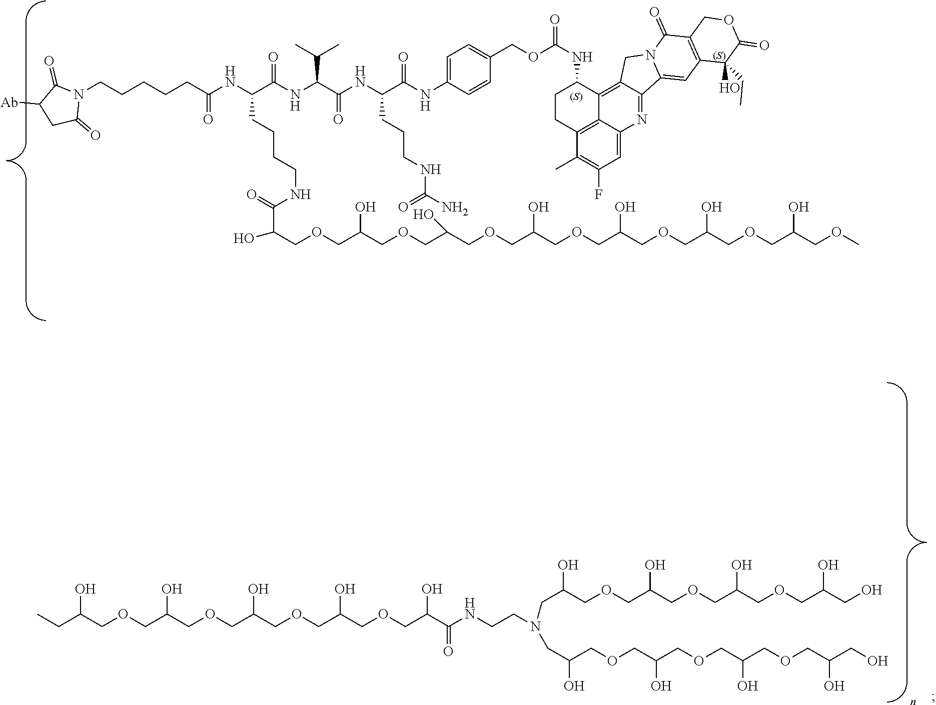

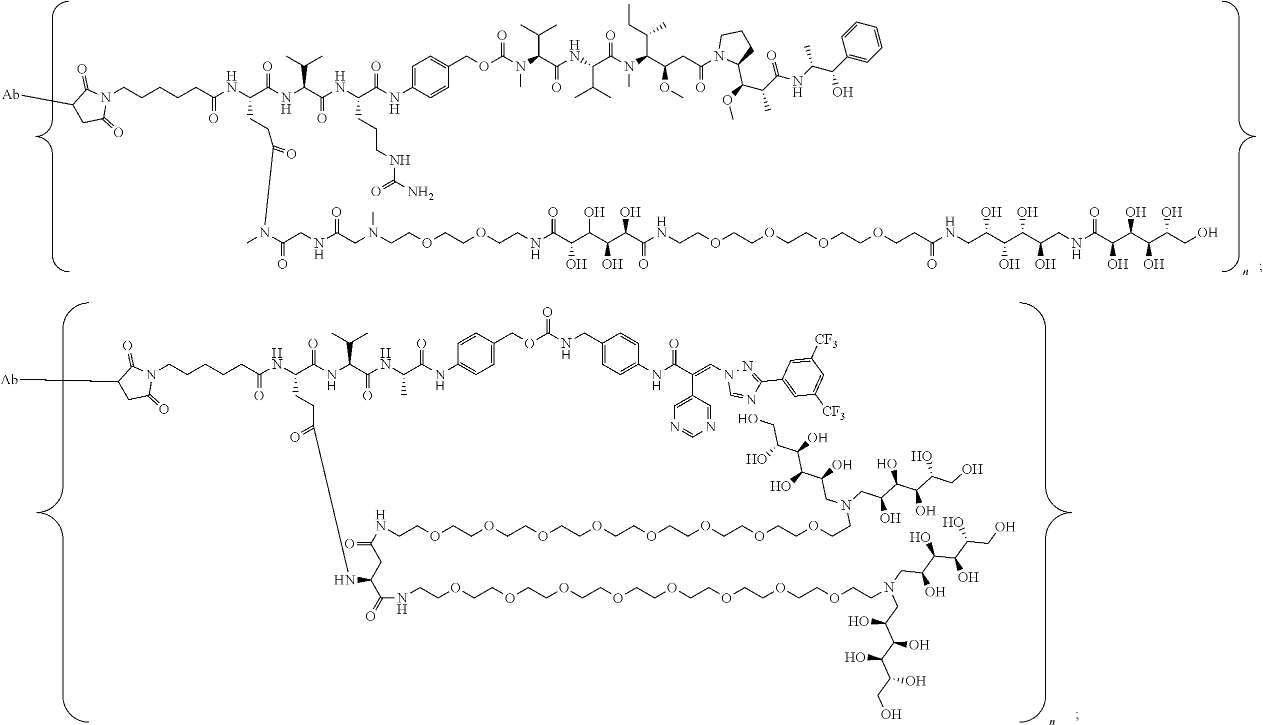

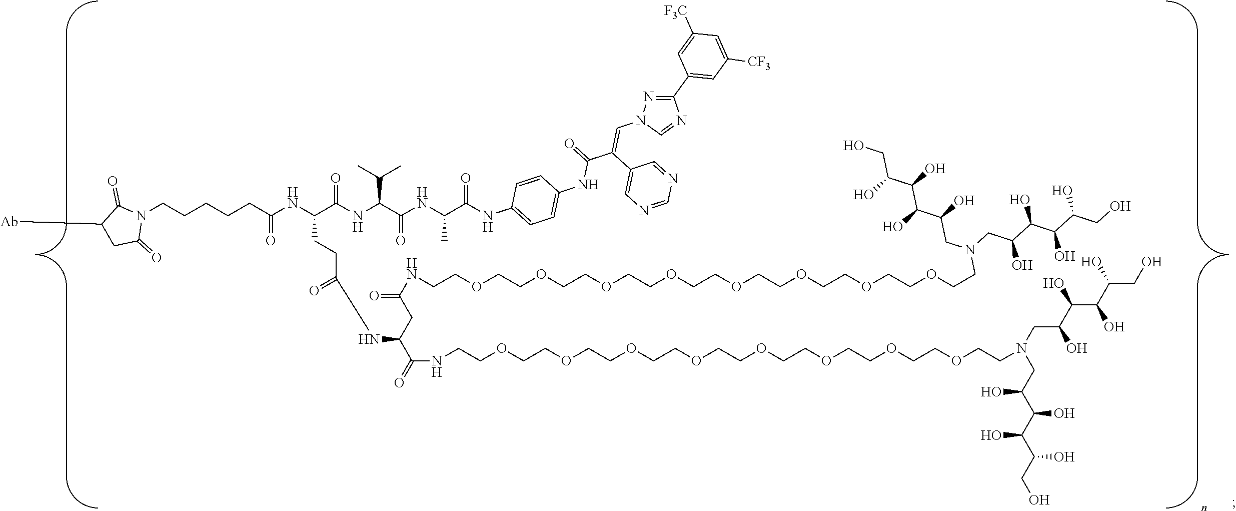

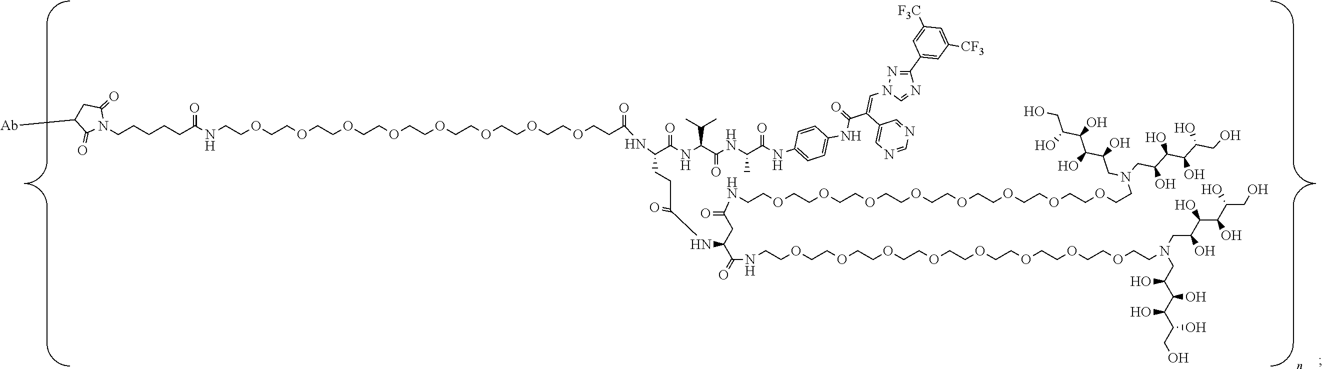

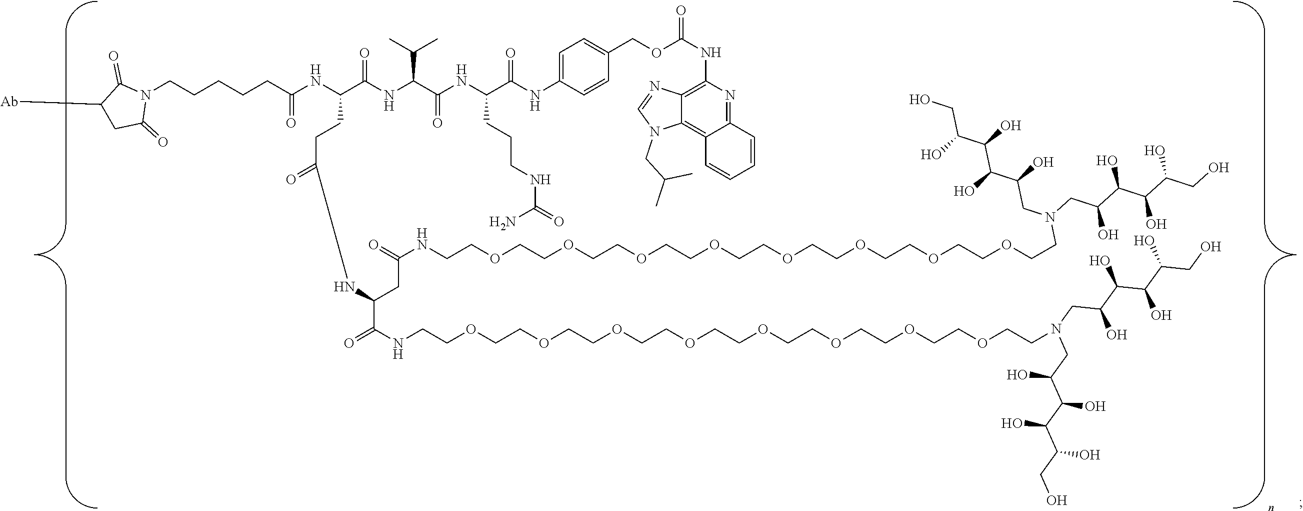

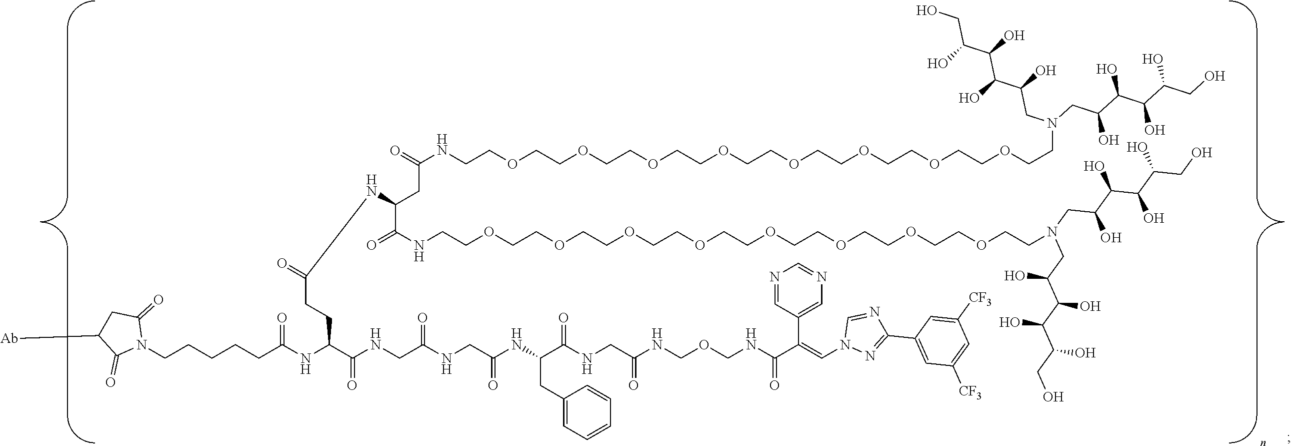

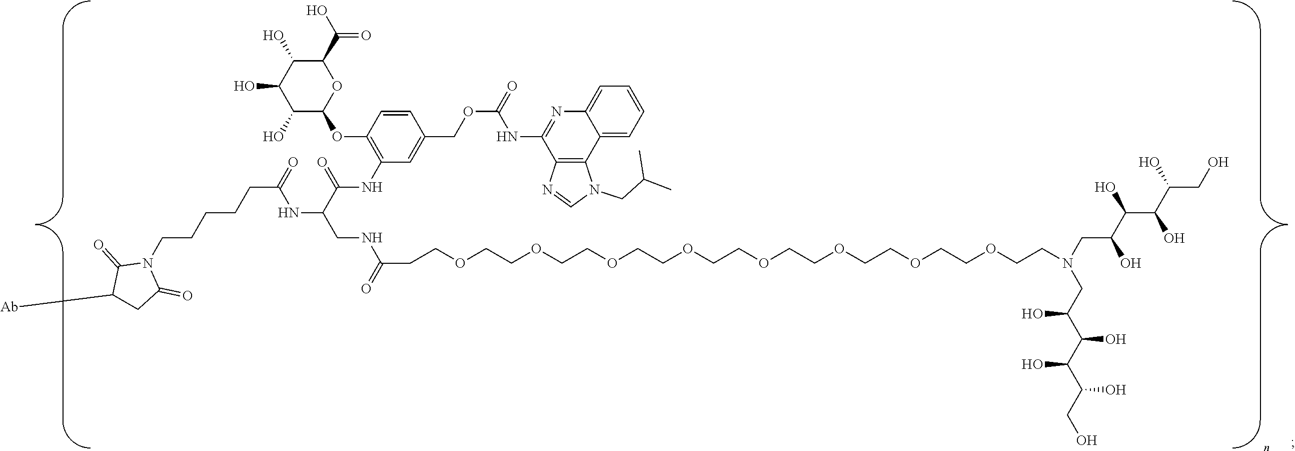

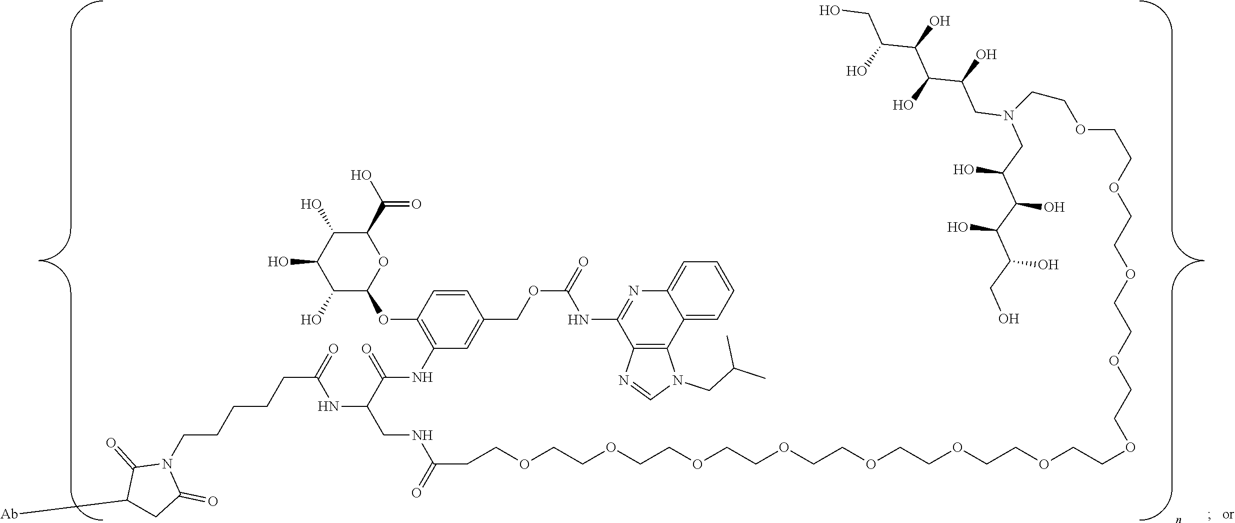

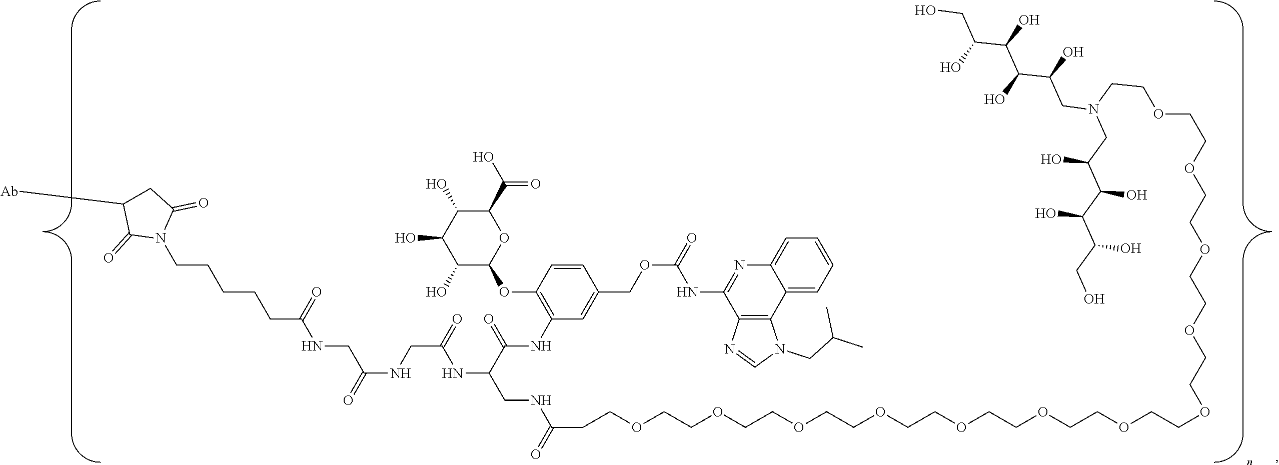

or a stereoisomer thereof, wherein Ab is a SLITRK6 binding agent and n is p^{load}.

Embodiment 141. The conjugate of any one of Embodiments 137-138, selected from the following:

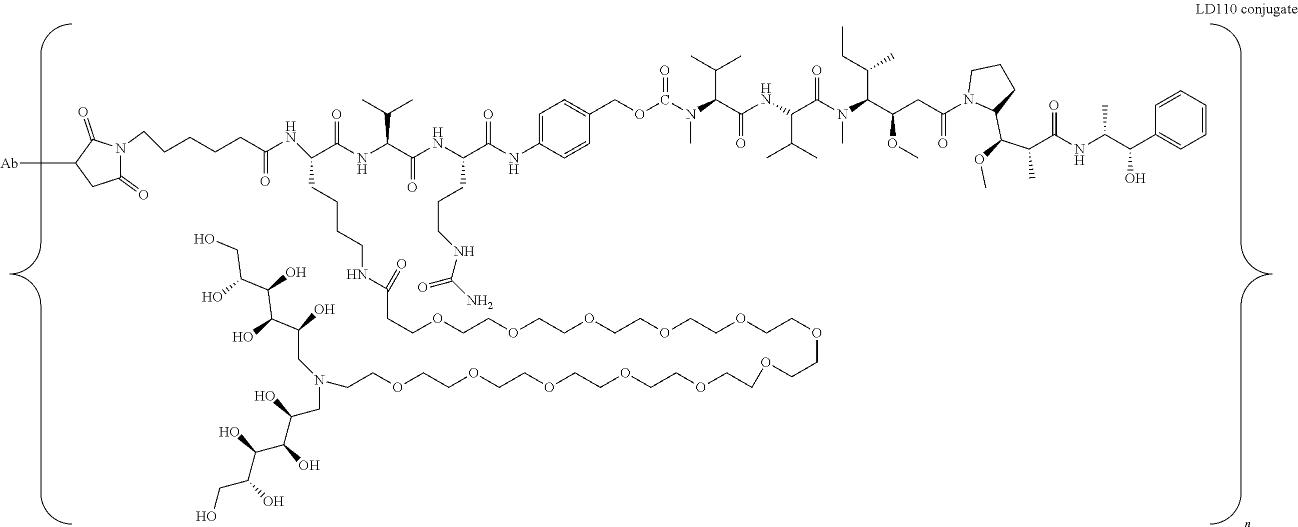

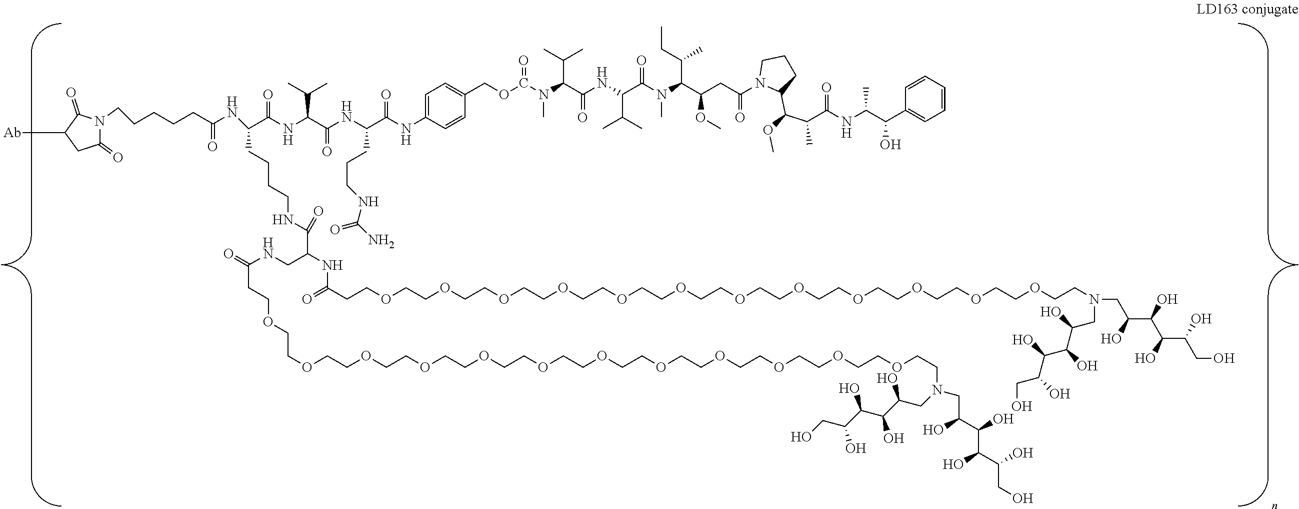

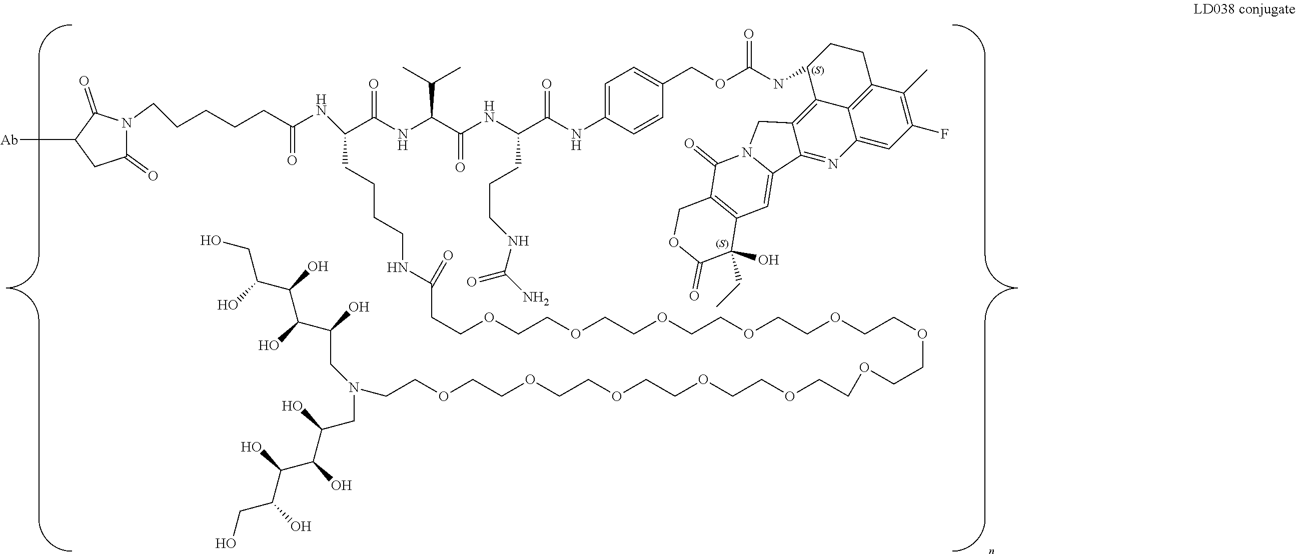

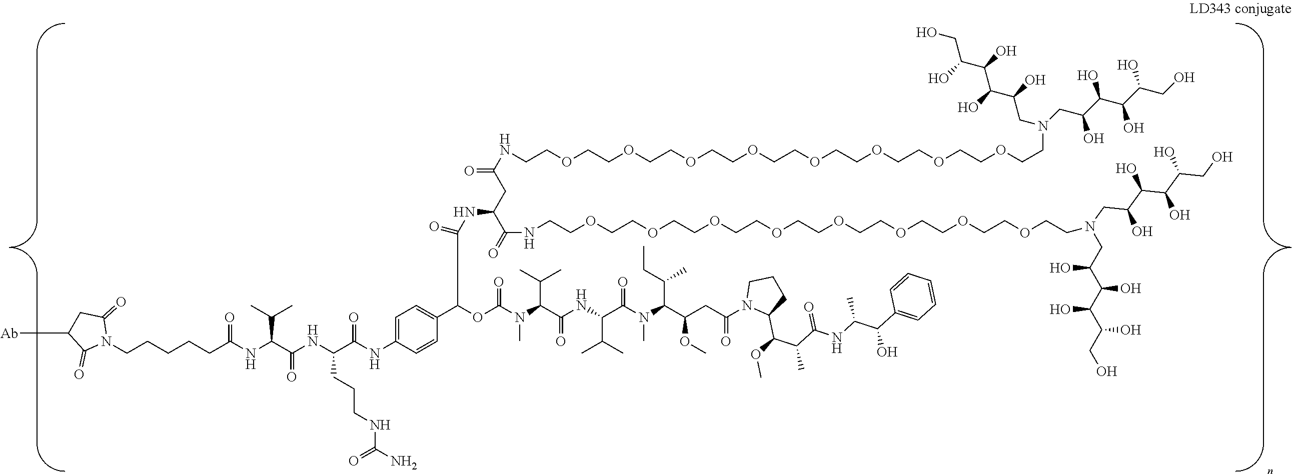

or a stereoisomer thereof, wherein Ab is a SLITRK6 binding agent and n is p^{load}.

Embodiment 142. The conjugate of any one of Embodiments 137-141, wherein the Ab is a SLITRK6 binding agent (e.g., an antibody or antigen-binding portion thereof which binds to SLITRK6) comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of:
(i) SEQ ID NO: 3, SEQ ID NO: 4, SEQ ID NO: 5, SEQ ID NO: 6, SEQ ID NO: 7, and SEQ ID NO: 8, respectively;
(ii) SEQ ID NO: 11, SEQ ID NO: 12, SEQ ID NO: 13, SEQ ID NO: 14, SEQ ID NO: 15 and SEQ ID NO: 16, respectively;
(iii) SEQ ID NO: 19, SEQ ID NO: 20, SEQ ID NO: 21, SEQ ID NO: 22, SEQ ID NO: 23 and SEQ ID NO: 24, respectively;
(iv) SEQ ID NO: 27, SEQ ID NO: 28, SEQ ID NO: 29, SEQ ID NO: 30, SEQ ID NO: 31 and SEQ ID NO: 32, respectively; and
(v) SEQ ID NO: 35, SEQ ID NO: 36, SEQ ID NO: 37, SEQ ID NO: 38, SEQ ID NO: 39 and SEQ ID NO: 40, respectively.

Embodiment 143. The conjugate of any one of Embodiments 137-142, wherein the VH and VL regions have amino acid sequences that are selected from the pairs of amino acid sequences set forth in the group consisting of:
(i) SEQ ID NO: 1 and SEQ ID NO: 2, respectively;
(ii) SEQ ID NO: 9 and SEQ ID NO: 10, respectively;
(iii) SEQ ID NO: 17 and SEQ ID NO: 18, respectively;
(iv) SEQ ID NO: 25 and SEQ ID NO: 26, respectively; and
(v) SEQ ID NO: 33 and SEQ ID NO: 34, respectively.

Embodiment 144. The conjugate of any one of Embodiments 137-142, wherein the VH and VL regions have amino acid sequences that are selected from the pairs of amino acid sequences set forth in the group consisting of:

(i) SEQ ID NO: 1 and SEQ ID NO: 2, respectively;
(ii) SEQ ID NO: 9 and SEQ ID NO: 10, respectively;
(iii) SEQ ID NO: 17 and SEQ ID NO: 18, respectively;
(iv) SEQ ID NO: 25 and SEQ ID NO: 26, respectively; and
(v) SEQ ID NO: 33 and SEQ ID NO: 34, respectively;
   wherein the heavy and light chain framework regions are optionally modified with from 1 to 8 amino acid substitutions, deletions or insertions in the framework regions.

Embodiment 145. The conjugate of any one of Embodiments 137-144, wherein the framework regions are human framework regions.

Embodiment 146. The conjugate of any one of Embodiments 137-145, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

Embodiment 147. The conjugate of any one of Embodiments 137-146, wherein the heavy chain variable region further comprises a heavy chain constant region.

Embodiment 148. The conjugate of any one of Embodiments 137-147, wherein the heavy chain constant region is of the IgG isotype.

Embodiment 149. The conjugate of Embodiment 148, wherein the heavy chain constant region is an IgG1 constant region.

Embodiment 150. The conjugate of Embodiment 148, wherein the heavy chain constant region is an IgG4 constant region.

Embodiment 151. The conjugate of Embodiment 147, wherein the heavy chain constant region comprises the amino acid sequence of SEQ ID NO: 49, 51, or 52.

Embodiment 152. The conjugate of any one of Embodiments 137-151, wherein the light chain variable region further comprises a light chain constant region.

Embodiment 153. The conjugate of Embodiment 152, wherein the light chain constant region is of the kappa isotype.

Embodiment 154. The conjugate of Embodiment 153, wherein the light chain constant region has the amino acid sequence set forth in SEQ ID NO: 50.

Embodiment 155. The conjugate of any one of Embodiments 147-154, wherein the heavy chain constant region further comprises an amino acid modification that decreases binding affinity to human FcγRIII.

Embodiment 156. The conjugate of any one of Embodiments 137-155, wherein the binding agent comprises a heavy chain and a light chain comprising the amino acid sequences set forth in SEQ ID NOs: 53 and 55, respectively.

Embodiment 157. The conjugate of any one of Embodiments 137-155, wherein the binding agent comprises a heavy chain and a light chain comprising the amino acid sequences set forth in SEQ ID NOs: 54 and 55, respectively.

Embodiment 158. The conjugate of any one of Embodiments 137-157, wherein the binding agent is mono-specific.

Embodiment 159. The conjugate of any one of Embodiments 137-158, wherein the binding agent is bivalent.

Embodiment 160. The conjugate of any one of Embodiments 137-157 and 159, wherein the binding agent is bispecific.

Embodiment 161. A binding agent which binds to SLITRK6 comprising a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs having amino acids sequences selected from the sets of amino acid sequences set forth in the group consisting of:
(i) SEQ ID NO: 3, SEQ ID NO: 4, SEQ ID NO: 5, SEQ ID NO: 6, SEQ ID NO: 7, and SEQ ID NO: 8, respectively;
(ii) SEQ ID NO: 11, SEQ ID NO: 12, SEQ ID NO: 13, SEQ ID NO: 14, SEQ ID NO: 15 and SEQ ID NO: 16, respectively;
(iii) SEQ ID NO: 19, SEQ ID NO: 20, SEQ ID NO: 21, SEQ ID NO: 22, SEQ ID NO: 23 and SEQ ID NO: 24, respectively;
(iv) SEQ ID NO: 27, SEQ ID NO: 28, SEQ ID NO: 29, SEQ ID NO: 30, SEQ ID NO: 31 and SEQ ID NO: 32, respectively; and
(v) SEQ ID NO: 35, SEQ ID NO: 36, SEQ ID NO: 37, SEQ ID NO: 38, SEQ ID NO: 39 and SEQ ID NO: 40, respectively.

Embodiment 162. The binding agent of Embodiment 161, wherein the VH and VL regions have amino acid sequences that are selected from the pairs of amino acid sequences set forth in the group consisting of:
(i) SEQ ID NO: 1 and SEQ ID NO: 2, respectively;
(ii) SEQ ID NO: 9 and SEQ ID NO: 10, respectively;
(iii) SEQ ID NO: 17 and SEQ ID NO: 18, respectively;
(iv) SEQ ID NO: 25 and SEQ ID NO: 26, respectively; and
(v) SEQ ID NO: 33 and SEQ ID NO: 34, respectively.

Embodiment 163. The binding agent of Embodiment 161, wherein the VH and VL regions have amino acid sequences that are selected from the pairs of amino acid sequences set forth in the group consisting of:
SEQ ID NO: 1 and SEQ ID NO: 2, respectively;
SEQ ID NO: 9 and SEQ ID NO: 10, respectively;
SEQ ID NO: 17 and SEQ ID NO: 18, respectively;
SEQ ID NO: 25 and SEQ ID NO: 26, respectively; and
SEQ ID NO: 33 and SEQ ID NO: 34, respectively;
wherein the heavy and light chain framework regions are optionally modified with from 1 to 8 amino acid substitutions, deletions or insertions in the framework regions.

Embodiment 164. The binding agent of Embodiment 161, wherein the framework regions are human framework regions.

Embodiment 165. The binding agent of any of Embodiments 161-164, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

Embodiment 166. The binding agent of any of Embodiments 161-165, wherein the VH region further comprises a heavy chain constant region.

Embodiment 167. The binding agent of Embodiment 166, wherein the heavy chain constant region is of the IgG isotype.

Embodiment 168. The binding agent of Embodiment 167, wherein the heavy chain constant region is an IgG1 constant region.

Embodiment 169. The binding agent of Embodiment 167, wherein the heavy chain constant region is an IgG4 constant region.

Embodiment 170. The binding agent of Embodiment 167, wherein the heavy chain constant region comprises the amino acid sequence set forth in SEQ ID NO: 49, 51, or 52.

Embodiment 171. The binding agent of any of the Embodiments 161-170, wherein the VL region further comprises a light chain constant region.

Embodiment 172. The binding agent of Embodiment 171, wherein the light chain constant region is of the kappa isotype.

Embodiment 173. The binding agent of Embodiment 172, wherein the light chain constant region comprises the amino acid sequence set forth in SEQ ID NO: 50.

Embodiment 174. The binding agent of any one of Embodiments 166-173, wherein the heavy chain constant region further comprises at least amino acid modification that decreases binding affinity to human FcγRIII.

Embodiment 175. The binding agent of any one of Embodiments 161-174, wherein the binding agent comprises a heavy chain and a light chain comprising the amino acid sequences set forth in SEQ ID NOs: 53 and 55, respectively.

Embodiment 176. The binding agent of any one of Embodiments 161-174, wherein the binding agent comprises a heavy chain and a light chain comprising the amino acid sequences set forth in SEQ ID NOs: 54 and 55, respectively.

Embodiment 177. The binding agent of any of Embodiments 161-176, wherein the binding agent is mono-specific.

Embodiment 178. The binding agent of any one of Embodiments 161-177, wherein the binding agent is bivalent.

Embodiment 179. The binding agent of any one of Embodiments 161-176 and 178, wherein the binding agent is bispecific.

Embodiment 180. A nucleic acid, or set of nucleic acids, encoding the binding agent of any one of Embodiments 161-179.

Embodiment 181. A vector, or set of vectors, comprising the nucleic acid, or set of nucleic acids, of Embodiment 180.

Embodiment 182. A cell line (e.g., an isolated cell line) comprising the nucleic acid, or set of nucleic acids, of Embodiment 180.

Embodiment 183. A cell line (e.g., an isolated cell line) comprising the vector, or set of vectors, of Embodiment 181.

Embodiment 184. A pharmaceutical composition comprising the conjugate of any one of Embodiments 137-160 or binding agent of any one of Embodiments 161-179, and a pharmaceutically acceptable carrier.

Embodiment 185. A method of treating a SLITRK6+ cancer, comprising administering to a subject in need thereof a therapeutically effective amount of the conjugate of any one of Embodiments 137-160, binding agent of any one of Embodiments 161-179, or the pharmaceutical composition of Embodiment 184.

Embodiment 186. The method of Embodiment 185, wherein the SLITRK6+ cancer is a solid tumor or a hematologic malignancy.

Embodiment 187. The method of Embodiment 186, wherein the SLITRK6+ cancer is selected from breast cancer (BC), lung cancer (LC), ovarian cancer (OVCA), esophageal cancer (EsC), gastric cancer (GC), bladder cancer (BLC), endometrial cancer (EC), head and neck cancer (HNC), cervical cancer, pharynx cancer, stomach cancer, myeloma, uterine cancer, colon cancer, hepatocellular cancer, and colorectal cancer.

Embodiment 188. The method of Embodiment 186, wherein the SLITRK6+ cancer is a hematologic malignancy.

Embodiment 189. The method of Embodiment 186, wherein the SLITRK6+ cancer is a solid tumor.

Embodiment 190. The method of Embodiment 186, wherein the SLITRK6+ cancer is triple-negative breast cancer (TNBC).

Embodiment 191. The method of Embodiment 186, wherein the SLITRK6+ cancer is non-small-cell lung cancer (NSCLC).

Embodiment 192. The method of Embodiment 186, wherein the SLITRK6+ cancer is ovarian cancer.

Embodiment 193. The method of Embodiment 186, wherein the SLITRK6+ cancer is pharynx cancer.

Embodiment 194. The method of Embodiment 186, wherein the SLITRK6+ cancer is gastric cancer.

Embodiment 195. The method of Embodiment 186, wherein the SLITRK6+ cancer is endometrial adenocarcinoma.

Embodiment 196. The method of Embodiment 186, wherein the SLITRK6+ cancer is bladder cancer.

Embodiment 197. The method of Embodiment 186, wherein the SLITRK6+ cancer is bladder transitional cell papilloma.

Embodiment 198. The method of Embodiment 186, wherein the SLITRK6+ cancer is EsCC.

Embodiment 199. The method of any one of Embodiments 185-198, further comprising administering an immunotherapy to the subject.

Embodiment 200. The method of Embodiment 199, wherein the immunotherapy comprises a checkpoint inhibitor.

Embodiment 201. The method of Embodiment 200, wherein the checkpoint inhibitor is selected from an antibody that specifically binds to human PD-1, human PD-L1, or human CTLA4.

Embodiment 202. The method of Embodiment 200, wherein the checkpoint inhibitor is pembrolizumab, nivolumab, cemiplimab or ipilimumab.

Embodiment 203. The method of any one of Embodiments 185-202, further comprising administering chemotherapy to the subject.

Embodiment 204. The method of any one of Embodiments 185-203, wherein the conjugate or pharmaceutical composition is administered intravenously.

Embodiment 205. The method of Embodiment 204, wherein the conjugate or pharmaceutical composition is administered in a dose of about 0.1 mg/kg to about 12 mg/kg.

Embodiment 206. The method of any one of Embodiments 185-205, wherein a treatment outcome of the subject is improved.

Embodiment 207. The method of Embodiment 206, wherein the improved treatment outcome is an objective response selected from stable disease, a partial response or a complete response.

Embodiment 208. The method of Embodiment 207, wherein the improved treatment outcome is reduced tumor burden.

Embodiment 209. The method of Embodiment 207, wherein the improved treatment outcome is progression-free survival or disease-free survival.

Embodiment 210. Use of the conjugate of any one of Embodiments 137-160, the binding agent of any one of Embodiments 161-179, or the pharmaceutical composition of Embodiment 184 for the treatment of SLITRK6+ cancer in a subject.

Embodiment 211. A method of treating an autoimmune disease, comprising administering to a subject in need thereof a therapeutically effective amount of the conjugate of any one of Embodiments 137-160, the binding agent of any one of Embodiments 161-179, or the pharmaceutical composition of Embodiment 184.

Embodiment 212. The method of Embodiment 211, wherein the autoimmune disease is rheumatoid arthritis, multiple sclerosis, or systemic lupus erythematosus.

Embodiment 213. The method of Embodiment 211 or 212, further comprising administering an immunosuppressive therapy to the subject.

Embodiment 214. The method of any one of Embodiments 211-213, wherein the conjugate or pharmaceutical composition is administered intravenously.

Embodiment 215. The method of Embodiment 214, wherein the conjugate or pharmaceutical composition is administered in a dose of about 0.1 mg/kg to about 12 mg/kg.

Embodiment 216. The method of any one of Embodiments 211-215, wherein a treatment outcome of the subject is improved.

Embodiment 217. The method of Embodiment 216, wherein the improved treatment outcome is a reduction in disease progression or alleviation of disease severity.

Embodiment 218. Use of the conjugate of any one of Embodiments 137-160, the binding agent of any one of Embodiments 161-179, or the pharmaceutical composition of Embodiment 184 for the treatment of an autoimmune disease in a subject.

Embodiment 219. A method of producing the binding agent which binds to SLITRK6, the method comprising culturing the cell line of Embodiment 182 or 183 and isolating the antibody from the cell.

Embodiment 220. An anti-idiotypic antibody which binds to the binding agent of any one of Embodiments 161-179.

Embodiment 221. The conjugate of any one of Embodiments 137-160, the binding agent of any one of Embodiments 161-179, or the pharmaceutical composition of Embodiment 184 for use as a medicament.

Embodiment 222. The conjugate of any one of Embodiments 137-160, the binding agent of any one of Embodiments 161-179, or the pharmaceutical composition of Embodiment 184 for use in the treatment of cancer in a subject, preferably said cancer is SLITRK6+ cancer.

Embodiment 223. The conjugate of any one of Embodiments 137-160, the binding agent of any one of Embodiments 161-179, or the pharmaceutical composition of Embodiment 184 for use in the treatment of an autoimmune disease in a subject.

Embodiment 224. Use of the conjugate of any one of Embodiments 137-160, the binding agent of any one of Embodiments 161-179, or the pharmaceutical composition of Embodiment 184 for the manufacture of a medicament.

Embodiment 225. Use of the conjugate of any one of Embodiments 137-160, the binding agent of any one of Embodiments 161-179, or the pharmaceutical composition of Embodiment 184 for the manufacture of a medicament for the treatment of cancer in a subject, preferably said cancer is SLITRK6+ cancer.

Embodiment 226. Use of the conjugate of any one of Embodiments 137-160, the binding agent of any one of Embodiments 161-179, or the pharmaceutical composition of Embodiment 184 for the manufacture of a medicament for the treatment of an autoimmune disease in a subject.

### Drug Loading

Conjugates can contain one or more drug unit per SLITRK6 binding agent. The number of drug units per SLITRK6 binding agent is referred to as drug loading. The drug loading of a Conjugate is represented by p^{load}, the average number of drug units (drug molecules (e.g., cytotoxic agents)) per SLITRK6 binding agent (e.g., an antibody or antigen binding portion or non-antibody scaffold or non-antibody protein) in a conjugate. For example, if p^{load} is about 4, the average drug loading taking into account all of the SLITRK6 binding agent (e.g., antibodies or antigen binding portion or non-antibody scaffold or non-antibody proteins) present in the composition is about 4. In some embodiments, p^{load} ranges from about 3 to about 5, from about 3.6 to about 4.4, or from about 3.8 to about 4.2. In some embodiments, p^{load} can be about 3, about 4, or about 5. In some embodiments, p^{load} ranges from about 6 to about 8, more preferably from about 7.5 to about 8.4. In some embodiments, p^{load} can be about 6, about 7, or about 8. In some embodiments, p^{load} ranges from about 8 to about 16.

The average number of drug units per SLITRK6 binding agent (e.g., antibody or antigen binding portion or non-antibody scaffold) in a preparation may be characterized by conventional means such as UV, mass spectroscopy, Capillary Electrophoresis (CE), and HPLC. The quantitative distribution of conjugates in terms of p^{load} may also be determined. In some instances, separation, purification, and characterization of homogeneous conjugates where p^{load} is a certain value from conjugates with other drug loadings may be achieved by means such as reverse phase HPLC or Hydrophobic Interaction Chromatography (HIC) HPLC.

### Attachment of Drug-Linkers to Antibodies, Antigen Binding Portions and Other Binding Agents (Including Non-Antibody Scaffolds)

Techniques for attaching drug unit(s) to SLITRK6 binding agent (such as antibodies or antigen binding portions thereof or non-antibody scaffolds) via linkers are well-known in the art. See, e.g., Alley et al., Current Opinion in Chemical Biology 2010 14:1-9; Senter, Cancer J., 2008, 14(3):154-169. In some embodiments, a linker is first attached to a drug unit (e.g., a cytotoxic agent(s), immune modulatory agent or other agent) and then the drug-linker(s) is attached to the SLITRK6 binding agent (e.g., an antibody or antigen binding portion thereof or non-antibody protein scaffold). In some embodiments, a linker(s) is first attached to a SLITRK6 binding agent (e.g., an antibody or antigen binding portion thereof or non-antibody protein scaffold), and then a drug unit is attached to a linker. In the following discussion, the term drug-linker is used to exemplify attachment of linkers or drug-linkers to the SLITRK6 binding agent; the skilled artisan will appreciate that the selected attachment method can be determined according to linker and the drug unit. In some embodiments, a drug unit is attached to a SLITRK6 binding agent via a linker in a manner that reduces the activity of the drug unit until it is released from the conjugate (e.g., by hydrolysis, by proteolytic degradation or by a cleaving agent.).

Generally, a conjugate may be prepared by several routes employing organic chemistry reactions, conditions, and reagents known to those skilled in the art, including: (1) reaction of a nucleophilic group of a SLITRK6 binding agent (e.g., an antibody or antigen binding portion thereof or non-antibody protein scaffold) with a bivalent linker to form a targeting group-linker intermediate via a covalent bond, followed by reaction with a drug unit; and (2) reaction of a nucleophilic group of a drug unit with a bivalent linker, to form drug-linker, via a covalent bond, followed by reaction with a nucleophilic group of a SLITRK6 binding agent. Exemplary methods for preparing conjugates via the latter route are described in U.S. Pat. No. 7,498,298, which is expressly incorporated herein by reference.

Nucleophilic groups on the SLITRK6 binding agent such as antibodies, antigen binding portions and other binding agents (including non-antibody scaffolds) include, but are not limited to: (i)N-terminal amine groups, (ii) side chain amine groups, e.g. lysine, (iii) side chain thiol groups, e.g. cysteine, and (iv) sugar hydroxyl or amino groups where the antibody is glycosylated. Amine, thiol, and hydroxyl groups are nucleophilic and capable of reacting to form covalent bonds with electrophilic groups on linkers including: (i) active esters such as NHS esters, HOBt esters, haloformates, and acid halides; (ii) alkyl and benzyl halides such as haloacetamides; and (iii) aldehydes, ketones, carboxyl, and maleimide groups. The SLITRK6 binding agent, such as antibodies (and antigen binding portions and other binding agents (including non-antibody scaffolds)) has reducible interchain disulfides, i.e., cysteine bridges. Antibodies (and antigen binding portions and other binding agents (including non-antibody scaffolds)) may be made reactive for conjugation with linkers by treatment with a reducing agent such as DTT (dithiothreitol) or tricarbonylethylphosphine (TCEP), such that the antibody is fully or partially reduced. Each cysteine bridge will thus form, theoretically, two reactive thiol nucleophiles. Additional nucleophilic groups can be introduced into the SLITRK6 binding agent such as antibodies (and antigen binding portions and other binding agents (including non-antibody scaffolds)) through modification of lysine residues, e.g., by reacting lysine residues with 2-iminothiolane (Traut's reagent), resulting in conversion of an amine into a thiol. Reactive thiol groups may also be introduced into the SLITRK6 binding agent (such as an antibody and antigen binding portions and other binding agents (including non-antibody scaffolds)) by introducing one, two, three, four, or more cysteine residues (e.g., by preparing antibodies, antigen binding portions and other binding agents (including non-antibody scaffolds) comprising one or more non-native cysteine amino acid residues).

Conjugates may also be produced by reaction between an electrophilic group on the SLITRK6 binding agent, such as an aldehyde or ketone carbonyl group, with a nucleophilic group on a linker reagent. Useful nucleophilic groups on a linker reagent include, but are not limited to, hydrazide, oxime, amino, hydrazine, thiosemicarbazone, hydrazine carboxyl, and arylhydrazide. In an embodiment, an antibody (or antigen binding portion thereof or other binding agent (including non-antibody scaffolds)) is modified to introduce electrophilic moieties that are capable of reacting with nucleophilic substituents on a linker. In another embodiment, the sugars of glycosylated antibodies may be oxidized, e.g. with periodate oxidizing reagents, to form aldehyde or ketone groups which may react with the amine group of a linker. The resulting imine Schiff base groups may form a stable linkage, or may be reduced, e.g., by borohydride reagents to form stable amine linkages. In one embodiment, reaction of the carbohydrate portion of a glycosylated antibody with either galactose oxidase or sodium meta-periodate may yield carbonyl (aldehyde and ketone) groups in the antibody (or antigen binding portion thereof or other binding agent (including non-antibody scaffolds)) that can react with appropriate groups on the linker (see, e.g., Hermanson, Bioconjugate Techniques). In another embodiment, the SLITRK6 binding agent such as antibodies containing N-terminal serine or threonine residues can react with sodium meta-periodate, resulting in production of an aldehyde in place of the first amino acid (Geoghegan & Stroh, (1992) Bioconjugate Chem. 3:138-146; U.S. Pat. No. 5,362,852). Such an aldehyde can be reacted with a linker.

Exemplary nucleophilic groups on a drug unit, such as a cytotoxic agent, include, but are not limited to: amine, thiol, hydroxyl, hydrazide, oxime, hydrazine, thiosemicarbazone, hydrazine carboxyl, and arylhydrazide groups capable of reacting to form covalent bonds with electrophilic groups on a linker(s) including: (i) active esters such as NHS esters, HOBt esters, haloformates, and acid halides; (ii) alkyl and benzyl halides such as haloacetamides; (iii) aldehydes, ketones, carboxyl, and maleimide groups.

In some embodiments, a drug-linker is attached to an interchain cysteine residue(s) of an antibody (or antigen binding portion thereof or other binding agent (including non-antibody scaffolds)). See, e.g., WO2004/010957 and WO2005/081711. In such embodiments, the linker typically comprises a maleimide group for attachment to the cysteine residues of an interchain disulfide. In some embodiments, a linker or drug-linker is attached to a cysteine residue(s) of an antibody or antigen binding portion thereof as described in U.S. Pat. Nos. 7,585,491 or 8,080m250. The drug loading of the resulting conjugate typically ranges from 1 to 8 or 1 to 16.

In some embodiments, a linker or drug-linker is attached to a lysine or cysteine residue(s) of an antibody (or antigen binding portion thereof or other binding agent) as described in WO2005/037992 or WO2010/141566. The drug loading of the resulting conjugate typically ranges from 1 to 8.

In some embodiments, engineered cysteine residues, poly-histidine sequences, glycoengineering tags, or transglutaminase recognition sequences can be used for site-specific attachment of linkers or drug-linkers to antibodies or antigen binding portions thereof or other binding agents (including non-antibody scaffolds).

In some embodiments, a drug-linker(s) is attached to an engineered cysteine residue at an Fc residue other than an interchain disulfide. In some embodiments, a drug-linker(s) is attached to an engineered cysteine introduced into an IgG (typically an IgG1) at position 118, 221, 224, 227, 228, 230, 231, 223, 233, 234, 235, 236, 237, 238, 239, 240, 241, 243, 244, 245, 247, 249, 250, 258, 262, 263, 264, 265, 266, 267, 268, 269, 270, 271, 272, 273, 275, 276, 278, 280, 281, 283, 285, 286, 291, 292, 293, 294, 295, 296, 297, 298, 299, 300, 302, 305, 313, 318, 323, 324, 325, 327, 328, 329, 330, 331, 332, 333, 335, 336, 396, and/or 428, of the heavy chain and/or to a light chain at position 106, 108, 142 (light chain), 149 (light chain), and/or position V205, according to the EU numbering of Kabat. An exemplary substitution for site specific conjugation using an engineered cysteine is S239C (see, e.g., US 20100158909; numbering of the Fc region is according to the EU index).

In some embodiments, a linker or drug-linker(s) is attached to one or more introduced cysteine residues of an antibody (or antigen binding portion thereof or other binding agent (including non-antibody scaffolds)) as described in WO2006/034488, WO2011/156328 and/or WO2016040856.

In some embodiments, an exemplary substitution for site specific conjugation using bacterial transglutaminase is N297S or N297Q of the Fc region. In some embodiments, a linker or drug-linker(s) is attached to the glycan or modified glycan of an antibody or antigen binding portion or a glycoengineered antibody (or other binding agent (including non-antibody scaffolds)). See, e.g., WO2017/147542, WO2020/123425, WO2020/245229, WO2014/072482; WO2014/065661, WO2015/057066 and WO2016/022027; the disclosure of which are incorporated by reference herein.

In some embodiments, a linker or drug-linker is attached to an antibody, antigen binding portion or other binding agent (including non-antibody scaffolds) via Sortase A linker. A Sortase A linker can be created by a Sortase A enzyme fusing an LPXTG recognition motif (SEQ ID NO: 58) to an N-terminal GGG motif to regenerate a native amide bond.

In some embodiments, a linker or drug-linker is attached to an antibody, antigen binding portion or other binding agent (including non-antibody scaffolds) using SMARTag Technology, in which a bioorthogonal aldehyde handle is introduced through the oxidation of a cysteine residue, embedded in a specific peptide sequence (CxPxR), to an aldehyde-bearing formylglycine (fGly). This enzymatic modification is carried out by the formylglycine-generating enzyme (FGE). See, e.g., Liu et al., Methods Mol. Biol. 2033:131-147 (2019).

In some embodiments, a linker or drug-linker is attached to an antibody, antigen binding portion or other binding agent (including non-antibody scaffolds) using cysteine conjugation with quaternized vinyl- and alkynyl-pyridine reagents. See, e.g., Matos et al., Angew Chem. Int. Ed. Engl. 58:6640-6644 (2019).

In other embodiments, a linker or drug-linker is attached to an antibody, antigen binding portion or other binding agent (including non-antibody scaffolds) using bis-maleimide, C-lock, or K-lock methodologies.

### Pharmaceutical Compositions

Other aspects of the conjugates relate to compositions comprising active ingredients, including any of the conjugates described herein. In some embodiments, the composition is a pharmaceutical composition. As used herein, the term “pharmaceutical composition” refers to an active agent in combination with a pharmaceutically acceptable carrier accepted for use in the pharmaceutical industry. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The preparation of a pharmacological composition that contains active ingredients dissolved or dispersed therein is well understood in the art and need not be limited based on any particular formulation. Typically such compositions are prepared as injectable either as liquid solutions or suspensions; however, solid forms suitable for rehydration, or suspensions, in liquid prior to use can also be prepared. A preparation can also be emulsified or presented as a liposome composition. A conjugate can be mixed with excipients that are pharmaceutically acceptable and compatible with the active ingredient and in amounts suitable for use in the therapeutic methods described herein. Suitable excipients are, for example, water, saline, dextrose, glycerol, ethanol or the like and combinations thereof. In addition, if desired, a pharmaceutical composition can contain minor amounts of auxiliary substances such as wetting or emulsifying agents, pH buffering agents and the like which enhance or maintain the effectiveness of the active ingredient (e.g., a conjugate).

The pharmaceutical compositions as described herein can include pharmaceutically acceptable salts of the components therein. Pharmaceutically acceptable salts include the acid addition salts (formed with the free amino groups of a polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like.

Physiologically tolerable carriers are well known in the art. Exemplary liquid carriers are sterile aqueous solutions that contain the active ingredients (e.g., a conjugate) and water, and may contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active agent that will be effective in the treatment of a particular disorder or condition will depend on the nature of the disorder or condition, and can be determined by standard clinical techniques.

In some embodiments, a pharmaceutical composition comprising a conjugate can be a lyophilisate.

In some embodiments, a syringe comprising a therapeutically effective amount of a conjugate is provided.

### Treatment Methods

In some embodiments, provided are methods of treating a subject, comprising administering to the subject a conjugate described herein or a pharmaceutical composition described herein. For example, in some embodiments the subject has cancer or an autoimmune disease and the conjugate binds to the target antigen associated with the cancer or autoimmune disease.

In some embodiments, provided are methods of treating cancer comprising administering a conjugate. In some embodiments, the subject is in need of treatment for a cancer and/or a malignancy. In some embodiments, the method is for treating a subject having a cancer or malignancy.

The methods described herein include administering a therapeutically effective amount of a conjugate to a subject having a cancer or malignancy. As used herein, the phrases “therapeutically effective amount”, “effective amount” or “effective dose” refer to an amount of a conjugate that provides a therapeutic benefit in the treatment of, management of or prevention of relapse of a cancer or malignancy, e.g., an amount that provides a statistically significant decrease in at least one symptom, sign, or marker of a tumor or malignancy. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The terms “cancer” and “malignancy” refer to an uncontrolled growth of cells which interferes with the normal functioning of the bodily organs and systems. A cancer or malignancy may be primary or metastatic, i.e. that is it has become invasive, seeding tumor growth in tissues remote from the original tumor site. A “tumor” refers to an uncontrolled growth of cells which interferes with the normal functioning of the bodily organs and systems. A subject that has a cancer is a subject having objectively measurable cancer cells present in the subject's body. Included in this definition are benign tumors and malignant cancers, as well as potentially dormant tumors and micro-metastases. Cancers that migrate from their original location and seed other vital organs can eventually lead to the death of the subject through the functional deterioration of the affected organs. Hematologic malignancies (hematopoietic cancers), such as leukemias and lymphomas, are able to, for example, out-compete the normal hematopoietic compartments in a subject, thereby leading to hematopoietic failure (in the form of anemia, thrombocytopenia and neutropenia) ultimately causing death.

Examples of cancers include, but are not limited to, carcinomas, lymphomas, blastomas, sarcomas, and leukemias. More particular examples of such cancers include, but are not limited to, basal cell cancer, biliary tract cancer, bladder cancer, bone cancer, brain and CNS cancer, breast cancer (e.g., triple negative breast cancer), cancer of the peritoneum, cervical cancer; cholangiocarcinoma, choriocarcinoma, chondrosarcoma, colon and rectum cancer (colorectal cancer), connective tissue cancer, cancer of the digestive system, endometrial cancer, esophageal cancer, eye cancer, cancer of the head and neck, gastric cancer (including gastrointestinal cancer and stomach cancer), glioblastoma (GBM), hepatic cancer, hepatoma, intra-epithelial neoplasm, kidney or renal cancer (e.g., clear cell cancer), larynx cancer, leukemia, liver cancer, lung cancer (e.g., small-cell lung cancer, non-small cell lung cancer, adenocarcinoma of the lung, and squamous cancer of the lung), lymphoma including Hodgkin's and non-Hodgkin's lymphoma, melanoma, mesothelioma, myeloma, neuroblastoma, oral cavity cancer (e.g., lip, tongue, mouth, and pharynx), ovarian cancer, pancreatic cancer, prostate cancer, retinoblastoma, rhabdomyosarcoma, cancer of the respiratory system, salivary gland cancer, sarcoma, skin cancer, squamous cell cancer, testicular cancer, thyroid cancer, uterine or endometrial cancer, uterine serious cancer, cancer of the urinary system, vulval cancer; as well as other carcinomas and sarcomas, as well as B-cell lymphoma (including low grade/follicular non-Hodgkin's lymphoma (NHL), small lymphocytic (SL) NHL, intermediate grade/follicular NHL, intermediate grade diffuse NHL, high grade immunoblastic NHL, high grade lymphoblastic NHL, high grade small non-cleaved cell NHL, bulky disease NHL, mantle cell lymphoma, AIDS-related lymphoma, and Waldenstrom's Macroglobulinemia), chronic lymphocytic leukemia (CLL), acute lymphoblastic leukemia (ALL), Hairy cell leukemia, chronic myeloblastic leukemia, and post-transplant lymphoproliferative disorder (PTLD), as well as abnormal vascular proliferation associated with phakomatoses, edema (such as that associated with brain tumors), and Meigs' syndrome.

In some embodiments, the cancer is a SLITRK6+ cancer. In some embodiments, the SLITRK6+ cancer is a solid tumor or a hematologic malignancy.

In some embodiments, the SLITRK6+ cancer is selected from breast cancer, ovarian cancer (OVCA), cervical cancer, pharynx cancer, stomach cancer, myeloma, bladder cancer, uterine cancer, esophageal squamous cell carcinoma (ESCC), colon cancer, hepatocellular cancer, and colorectal cancer.

In some embodiments, the SLITRK6+ cancer is a hematologic malignancy.

In some embodiments, SLITRK6+ cancer is a solid tumor. In some embodiments, the SLITRK6+ cancer is triple-negative breast cancer (TNBC). In some embodiments, the SLITRK6+ cancer is non-small-cell lung cancer (NSCLC). In some embodiments, the SLITRK6+ cancer is ovarian teratocarcinoma. In some embodiments, the SLITRK6+ cancer is pharynx cancer. In some embodiments, the SLITRK6+ cancer is gastric adenocarcinoma. In some embodiments, the SLITRK6+ cancer is endometrial adenocarcinoma. In some embodiments, the SLITRK6+ cancer is bladder transitional cell carcinoma. In some embodiments, the SLITRK6+ cancer is bladder transitional cell papilloma. In some embodiments, the SLITRK6+ cancer is ESCC.

It is contemplated that the methods herein reduce tumor size or tumor burden in the subject, and/or reduce metastasis in the subject. In various embodiments, tumor size in the subject is decreased by about 25-50%, about 40-70% or about 50-90% or more. In various embodiments, the methods reduce the tumor size by 10%, 20%, 30% or more. In various embodiments, the methods reduce tumor size by 10%, 15%, 20%, 25%, 30%, 35%, 40%, 45%, 50%, 55%, 60%, 65%, 70%, 75%, 80%, 85%, 90%, 95% or 100%.

In some embodiments, the improved treatment outcome is an objective response selected from stable disease, a partial response or a complete response. In some embodiments, the improved treatment outcome is progression-free survival or disease-free survival.

As used herein, a “subject” refers to a human or animal. Usually the animal is a vertebrate such as a primate, rodent, domestic animal or game animal. Primates include chimpanzees, cynomolgus monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. In certain embodiments, the subject is a mammal, e.g., a primate, e.g., a human. The terms, “patient”, “individual” and “subject” are used interchangeably herein.

Preferably, the subject is a mammal. The mammal can be a human, non-human primate, mouse, rat, dog, cat, horse, or cow, but are not limited to these examples. Mammals other than humans can be advantageously used, for example, as subjects that represent animal models of, for example, various cancers. In addition, the methods described herein can be used to treat domesticated animals and/or pets. A subject can be male or female. In certain embodiments, the subject is a human.

In some embodiments, a subject can be one who has been previously diagnosed with or identified as suffering from a cancer and in need of treatment, but need not have already undergone treatment for the cancer. In some embodiments, a subject can also be one who has not been previously diagnosed as having a cancer in need of treatment. In some embodiments, a subject can be one who exhibits one or more risk factors for a condition or one or more complications related to a cancer or a subject who does not exhibit risk factors. A “subject in need” of treatment for a cancer particular can be a subject having that condition or diagnosed as having that condition. In other embodiments, a subject “at risk of developing” a condition refers to a subject diagnosed as being at risk for developing the condition or at risk for having the condition again.

As used herein, the terms “treat,” “treatment,” “treating,” or “amelioration” when used in reference to a disease, disorder or medical condition, refer to therapeutic treatments for a condition, wherein the object is to reverse, alleviate, ameliorate, inhibit, slow down or stop the progression or severity of a symptom or condition. The term “treating” includes reducing or alleviating at least one adverse effect or symptom of a condition. Treatment is generally “effective” if one or more symptoms or clinical markers are reduced. Alternatively, treatment is “effective” if the progression of a condition is reduced or halted. That is, “treatment” includes not just the improvement of symptoms or markers, but also a cessation or at least slowing of progress or worsening of symptoms that would be expected in the absence of treatment.

Beneficial or desired clinical results include, but are not limited to, reduction in cancer cells in the subject, alleviation of one or more symptom(s), diminishment of extent of the deficit, stabilized (i.e., not worsening) state of a cancer or malignancy, delay or slowing of tumor growth and/or metastasis, and an increased lifespan as compared to that expected in the absence of treatment. As used herein, the term “administering,” refers to providing a conjugate as described herein to a subject by a method or route which results in binding of the conjugate to cancer cells or malignant cells. Similarly, a pharmaceutical composition comprising a conjugate as described herein can be administered by any appropriate route which results in an effective treatment in the subject.

The dosage ranges for a conjugate depend upon the potency, and encompass amounts large enough to produce the desired effect e.g., slowing of tumor growth or a reduction in tumor size. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.1 mg/kg to about 12 mg/kg. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

Pharmaceutical compositions containing a conjugate can be administered in a unit dose. The term “unit dose” when used in reference to a pharmaceutical composition refers to physically discrete units suitable as unitary dosage for the subject, each unit containing a predetermined quantity of active material (e.g., conjugate), calculated to produce the desired therapeutic effect in association with the required physiologically acceptable diluent, i.e., carrier, or vehicle.

In some embodiments, a conjugate, or a pharmaceutical composition of any of these, is administered with an additional chemotherapy or radiotherapy.

In some embodiments, the conjugates as described herein can be used in a method(s) comprising administering a conjugate to a subject in need thereof, such as a subject having an autoimmune disease.

In some embodiments, provided are methods of treating an autoimmune disease comprising administering a conjugate as described herein. In some embodiments, the subject is in need of treatment for an autoimmune disease. The methods described herein include administering a therapeutically effective amount of a conjugate to a subject having an autoimmune disease. As used herein, the phrase “therapeutically effective amount”, “effective amount” or “effective dose” refers to an amount of a conjugate as described herein that provides a therapeutic benefit in the treatment of, management of or prevention of relapse of an autoimmune disease, e.g., an amount that provides a statistically significant decrease in at least one symptom, sign, or marker of an autoimmune disease. Determination of a therapeutically effective amount is well within the capability of those skilled in the art. Generally, a therapeutically effective amount can vary with the subject's history, age, condition, sex, as well as the severity and type of the medical condition in the subject, and administration of other pharmaceutically active agents.

The term “autoimmune disease” refers to an immunological disorder characterized by inappropriate activation of immune cells (e.g., lymphocytes or dendritic cells), that interferes with the normal functioning of the bodily organs and systems. Examples of autoimmune disease include, but are not limited to, rheumatoid arthritis, psoriatic arthritis, autoimmune demyelinative diseases (e.g., multiple sclerosis, allergic encephalomyelitis), endocrine ophthalmopathy, uveoretinitis, systemic lupus erythematosus, myasthenia gravis, Grave's disease, glomerulonephritis, autoimmune hepatological disorder, inflammatory bowel disease (e.g., Crohn's disease), anaphylaxis, allergic reaction, Sjogren's syndrome, type I diabetes mellitus, primary biliary cirrhosis, Wegener's granulomatosis, fibromyalgia, polymyositis, dermatomyositis, multiple endocrine failure, Schmidt's syndrome, autoimmune uveitis, Addison's disease, adrenalitis, thyroiditis, Hashimoto's thyroiditis, autoimmune thyroid disease, pernicious anemia, gastric atrophy, chronic hepatitis, lupoid hepatitis, atherosclerosis, subacute cutaneous lupus erythematosus, hypoparathyroidism, Dressler's syndrome, autoimmune thrombocytopenia, idiopathic thrombocytopenic purpura, hemolytic anemia, pemphigus vulgaris, pemphigus, dermatitis herpetiformis, alopecia areata, pemphigoid, scleroderma, progressive systemic sclerosis, CREST syndrome (calcinosis, Raynaud's phenomenon, esophageal dysmotility, sclerodactyl), and telangiectasia), male and female autoimmune infertility, ankylosing spondolytis, ulcerative colitis, mixed connective tissue disease, polyarteritis nodosa, systemic necrotizing vasculitis, atopic dermatitis, atopic rhinitis, Goodpasture's syndrome, Chagas' disease, sarcoidosis, rheumatic fever, asthma, recurrent abortion, anti-phospholipid syndrome, farmer's lung, erythema multiforme, post cardiotomy syndrome, Cushing's syndrome, autoimmune chronic active hepatitis, bird-fancier's lung, toxic epidermal necrolysis, Alport's syndrome, alveolitis, allergic alveolitis, fibrosing alveolitis, interstitial lung disease, erythema nodosum, pyoderma gangrenosum, transfusion reaction, Takayasu's arteritis, polymyalgia rheumatica, temporal arteritis, schistosomiasis, giant cell arteritis, ascariasis, aspergillosis, Samter's syndrome, eczema, lymphomatoid granulomatosis, Behcet's disease, Caplan's syndrome, Kawasaki's disease, dengue, encephalomyelitis, endocarditis, endomyocardial fibrosis, endophthalmitis, erythema elevatum et diutinum, psoriasis, erythroblastosis fetalis, eosinophilic faciitis, Shulman's syndrome, Felty's syndrome, filariasis, cyclitis, chronic cyclitis, heterochronic cyclitis, Fuch's cyclitis, IgA nephropathy, Henoch-Schonlein purpura, graft versus host disease, transplantation rejection, cardiomyopathy, Eaton-Lambert syndrome, relapsing polychondritis, cryoglobulinemia, Waldenstrom's macroglobulemia, Evan's syndrome, and autoimmune gonadal failure.

In some embodiments, the methods provided herein are used to treat rheumatoid arthritis, multiple sclerosis, or systemic lupus erythematosus.

In some embodiments, the methods described herein encompass treatment of disorders of B lymphocytes (e.g., systemic lupus erythematosus, Goodpasture's syndrome, rheumatoid arthritis, and type I diabetes), Thi-lymphocytes (e.g., rheumatoid arthritis, multiple sclerosis, psoriasis, Sjorgren's syndrome, Hashimoto's thyroiditis, Grave's disease, primary biliary cirrhosis, Wegener's granulomatosis, tuberculosis, or graft versus host disease), or Th2-lymphocytes (e.g., atopic dermatitis, systemic lupus erythematosus, atopic asthma, rhinoconjunctivitis, allergic rhinitis, Omenn's syndrome, systemic sclerosis, or chronic graft versus host disease). Generally, disorders involving dendritic cells involve disorders of Th1-lymphocytes or Th2-lymphocytes.

In some embodiments, a subject can be one who has been previously diagnosed with or identified as suffering from an autoimmune disease and in need of treatment, but need not have already undergone treatment for the autoimmune disease. In some embodiments, a subject can also be one who has not been previously diagnosed as having an autoimmune disease in need of treatment. In some embodiments, a subject can be one who exhibits one or more risk factors for a condition or one or more complications related to an autoimmune disease or a subject who does not exhibit risk factors. A “subject in need” of treatment for an autoimmune disease particular can be a subject having that condition or diagnosed as having that condition. In other embodiments, a subject “at risk of developing” a condition refers to a subject diagnosed as being at risk for developing the condition or at risk for having the condition again (e.g., an autoimmune disease).

The dosage ranges for a conjugate depend upon the potency, and encompass amounts large enough to produce the desired effect e.g., slowing of progression of an autoimmune disease or a reduction of symptoms. The dosage should not be so large as to cause unacceptable adverse side effects. Generally, the dosage will vary with the age, condition, and sex of the subject and can be determined by one of skill in the art. The dosage can also be adjusted by the individual physician in the event of any complication. In some embodiments, the dosage ranges from 0.1 mg/kg body weight to 10 mg/kg body weight. In some embodiments, the dosage ranges from 0.5 mg/kg body weight to 15 mg/kg body weight. In some embodiments, the dose range is from 0.5 mg/kg body weight to 5 mg/kg body weight. Alternatively, the dose range can be titrated to maintain serum levels between 1 ug/mL and 1000 ug/mL. For systemic administration, subjects can be administered a therapeutic amount, such as, e.g. 0.1 mg/kg, 0.5 mg/kg, 1.0 mg/kg, 2.0 mg/kg, 2.5 mg/kg, 5 mg/kg, 10 mg/kg, 12 mg/kg or more.

Administration of the doses recited above can be repeated. In a preferred embodiment, the doses recited above are administered weekly, biweekly, every three weeks or monthly for several weeks or months. The duration of treatment depends upon the subject's clinical progress and responsiveness to treatment.

In some embodiments, a dose can be from about 0.1 mg/kg to about 100 mg/kg. In some embodiments, a dose can be from about 0.1 mg/kg to about 25 mg/kg. In some embodiments, a dose can be from about 0.1 mg/kg to about 20 mg/kg. In some embodiments, a dose can be from about 0.1 mg/kg to about 15 mg/kg. In some embodiments, a dose can be from about 0.1 mg/kg to about 12 mg/kg. In some embodiments, a dose can be from about 1 mg/kg to about 100 mg/kg. In some embodiments, a dose can be from about 1 mg/kg to about 25 mg/kg. In some embodiments, a dose can be from about 1 mg/kg to about 20 mg/kg. In some embodiments, a dose can be from about 1 mg/kg to about 15 mg/kg. In some embodiments, a dose can be from about 1 mg/kg to about 12 mg/kg. In some embodiments, a dose can be from about 1 mg/kg to about 10 mg/kg.

In some embodiments, a dose can be administered intravenously. In some embodiments, an intravenous administration can be an infusion occurring over a period of from about 10 minutes to about 4 hours. In some embodiments, an intravenous administration can be an infusion occurring over a period of from about 30 minutes to about 90 minutes.

In some embodiments, a dose can be administered weekly. In some embodiments, a dose can be administered bi-weekly. In some embodiments, a dose can be administered about every 2 weeks. In some embodiments, a dose can be administered about every 3 weeks. In some embodiments, a dose can be administered every four weeks.

In some embodiments, a total of from about 2 to about 10 doses are administered to a subject. In some embodiments, a total of 4 doses are administered. In some embodiments, a total of 5 doses are administered. In some embodiments, a total of 6 doses are administered. In some embodiments, a total of 7 doses are administered. In some embodiments, a total of 8 doses are administered. In some embodiments, a total of 9 doses are administered. In some embodiments, a total of 10 doses are administered. In some embodiments, a total of more than 10 doses are administered.

In some embodiments, a conjugate, or a pharmaceutical composition of any of these, is administered with an immunosuppressive therapy. In some embodiments, provided is a method of improving treatment outcome in a subject receiving immunosuppressive therapy. The method generally includes administering an effective amount of an immunosuppressive therapy to the subject having an autoimmune disorder; and administering a therapeutically effective amount of a conjugate or a pharmaceutical composition thereof to the subject, wherein the conjugate specifically binds to target autoimmune cells; wherein the treatment outcome of the subject is improved, as compared to administration of the immunotherapy alone. In some embodiments, the conjugate thereof as described herein. In some embodiments, an improved treatment outcome is a decrease in disease progression, an alleviation of one or more symptoms, or the like.

In some embodiments, the immunotherapy comprises a checkpoint inhibitor. In some embodiments, the checkpoint inhibitor is selected from an antibody that specifically binds to human PD-1, human PD-L1, or human CTLA4. In some embodiments, the checkpoint inhibitor is pembrolizumab, nivolumab, cemiplimab or ipilimumab.

The description of embodiments of the disclosure is not intended to be exhaustive or to limit the disclosure to the precise form disclosed. While specific embodiments of, and examples for, the disclosure are described herein for illustrative purposes, various equivalent modifications are possible within the scope of the disclosure, as those skilled in the relevant art will recognize. The teachings of the disclosure provided herein can be applied to other procedures or methods as appropriate. The various embodiments described herein can be combined to provide further embodiments. Aspects of the disclosure can be modified, if necessary, to employ the compositions, functions and concepts of the above references and application to provide yet further embodiments of the disclosure. These and other changes can be made to the disclosure in light of the detailed description.

Specific elements of any of the foregoing embodiments can be combined or substituted for elements in other embodiments. Furthermore, while advantages associated with certain embodiments of the disclosure have been described in the context of these embodiments, other embodiments may also exhibit such advantages, and not all embodiments need necessarily exhibit such advantages to fall within the scope of the disclosure. All patents and other publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### EXAMPLES

### Abbreviations

Boc₂O: di-tert-butyl dicarbonate
Bu₄NBr: Tetrabutylammonium bromide
DCM: dichloromethane
DEA: Diethanolamine
DEAD: diethyl azodicarboxylate
DIPEA: N,N-diisopropylethylamine
DMAP: 4-(Dimethylamino)pyridine
DMF: N,N-dimethylformamide
DMTMM: 4-(4,6-dimethoxy-1,3,5-triazin-2-yi)-4-methyl-morpholinium chloride
DMSO: dimethylsulfoxide
EEDQ: N-Ethoxycarbonyl-2-ethoxy-1,2-dihydroquinoline
ESI: electrospray ionization
HATU: 1-[bis(dimethyamino)methylene]-1H-1,2,3-triazolo[4,5-b]pyridinium 3-oxidhexa fluorophosophate)
HOBt: hydroxylbenzotriazole
LCMS: liquid chromatography-mass spectrometry
MeCN: acetonitrile
MeOH: methanol
m-CPBA: meta-chloroperoxybenzoic acid
MTBE: Methyl tert-butyl ether
NMR: nuclear magnetic resonance spectroscopy
Ph₃CCl: Triphenylmethyl chloride
PNPC: bis(4-nitrophenyl) carbonate
PPh₃: triphenylphosphine
TFA: trifluoroacetic acid
THF: tetrahydrofuran
TLC: thin-layer chromatography
TsOH: p-Toluenesulfonic acid

### General Methods

¹H NMR and other NMR spectra were recorded on Bruker AVIII 400 or Bruker AVIII 500. The data were processed with Nuts software or MestReNova software, measuring proton shifts in parts per million (ppm) downfield from an internal standard tetramethyl silane.

HPLC-MS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 15 min; Flow Rate: 1.0 mL/min; Column: XBridge C18, 4.6*150 mm, 3.5 um; Column Temperature: 40° C. Detectors: ADC ELSD, DAD (214 nm and 254 nm), ES-API.
Method B: Mobile Phase: A: Water (0.01% TFA) B: acetonitrile (0.01% TFA); Gradient Phase: 5% of B increasing to 95% of B in 15 min; Flow Rate: 1.0 mL/min; Column: SunFire C18, 4.6*150 mm, 3.5 μm; Column Temperature: 45° C. Detectors: ADC ELSD, DAD (214 nm and 254 nm), ES-API.
Method C: Mobile Phase: A: Water (10 mM NH₄HCO₃) B: acetonitrile; Gradient Phase: 5% to 95% of B in 15 min; Flow Rate: 1.0 mL/min; Column: XBridge C18, 4.6*150 mm, 3.5 μm; Column Temperature: 40° C. Detectors: ADC ELSD, DAD (214 nm and 254 nm), MSD (ES-API).

LCMS measurement was run on Agilent 1200 HPLC/6100 SQ System using the following conditions:
Method A: Mobile Phase: A: Water (0.01% TFA) B: acetonitrile (0.01% TFA); Gradient Phase: 5% of B increasing to 95% of B in 3 min; Flow Rate: 1.8-2.3 mL/min; Column: SunFire C18, 4.6*50 mm, 3.5 μm; Column Temperature: 50° C. Detectors: ADC ELSD, DAD (214 nm and 254 nm), ES-API.
Method B: Mobile Phase: A: Water (10 mM NH₄HCO₃) B: Acetonitrile; Gradient Phase: 5% to 95% of B in 3 min; Flow Rate: 1.8-2.3 mL/min; Column: XBridge C18, 4.6*50 mm, 3.5 μm; Column Temperature: 50° C. Detectors: ADC ELSD, DAD (214 nm and 254 nm), MSD (ES-API).

Preparative high pressure liquid chromatography (Prep-HPLC) was run on Gilson 281 using the following conditions:
Method A: Waters SunFire 10 μm C18 column (100 \acute{Å}, 250×19 mm). Solvent A was water/0.01% trifluoroacetic acid (TFA) and solvent B was acetonitrile. The elution condition was a linear gradient increase of solvent B from 5% to 100% over a time period of 20 minutes at a flow rate of 30 mL/min.
Method B: Waters SunFire 10 μm C18 column (100 \acute{Å}, 250×19 mm). Solvent A was water/0.05% formic acid (FA) and solvent B was acetonitrile. The elution condition was a linear gradient increase of solvent B from 5% to 100% over a time period of 20 minutes at a flow rate of 30 mL/min.
Method C: Waters Xbridge 10 μm C18 column (100 \acute{Å}, 250×19 mm). Solvent A was water/10 mM ammonium bicarbonate (NH₄HCO₃) and solvent B was acetonitrile. The elution condition was a linear gradient increase of solvent B from 5% to 100% over a time period of 20 minutes at a flow rate of 30 mL/min.

Flash chromatography was performed on instrument of Biotage, with Agela Flash Column silica-CS; Reverse phase flash chromatography was performed on instrument of Biotage, with Boston ODS or Agela C18.

### Example 1: Preparation of Drug-Linker 1

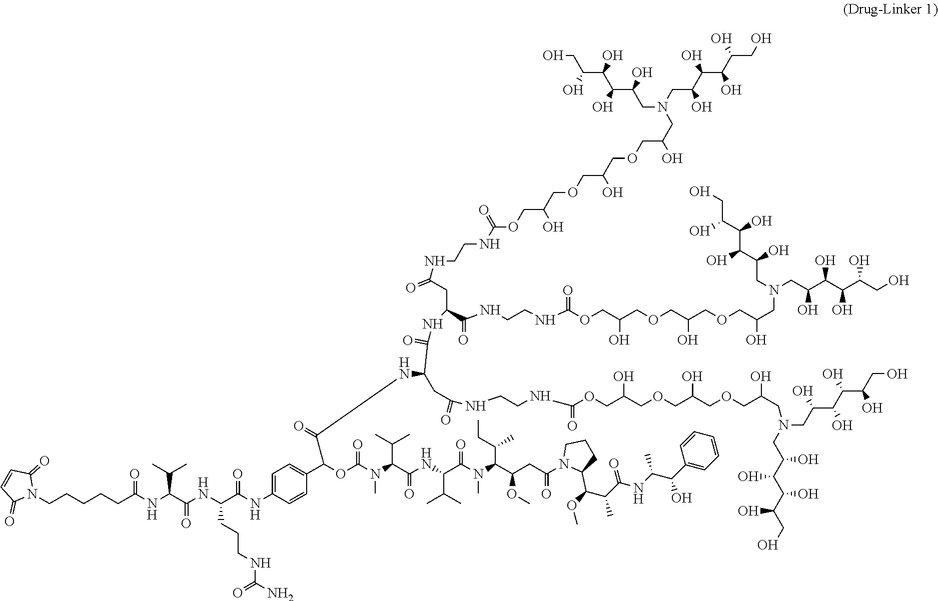

### Step 1

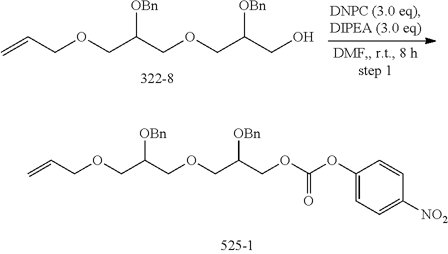 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 322-8 was consumed as detected by LCMS. The reaction solution was directly used in the next step without a work-up procedure.

### Step 2

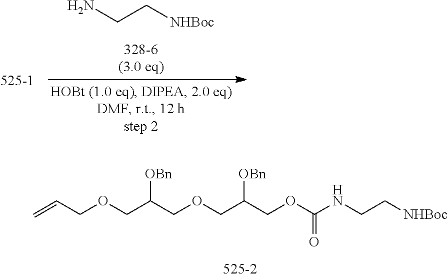 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed as detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

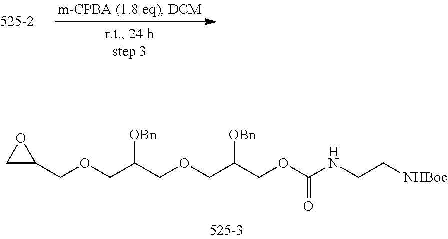 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil. Purity=90%-95%.

### Step 4

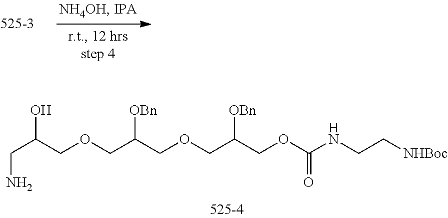 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

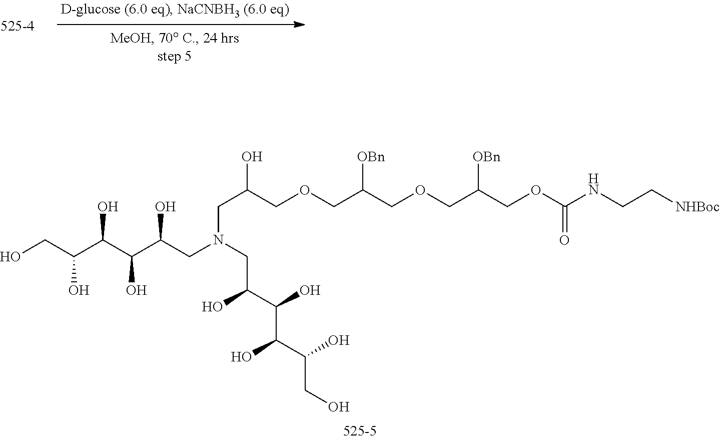 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

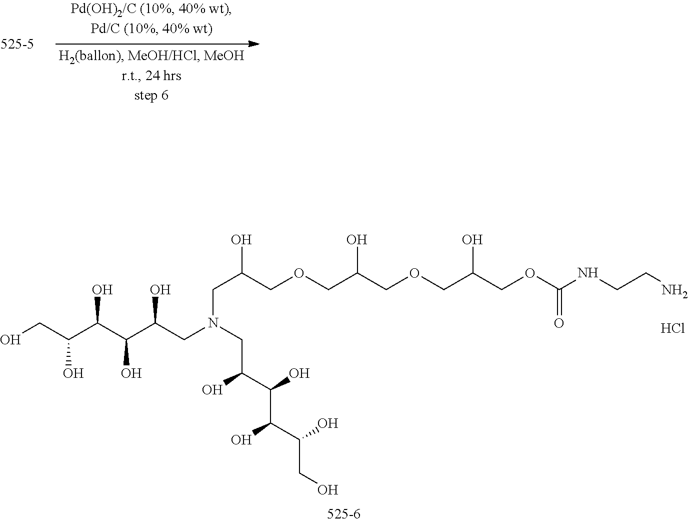 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (balloon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

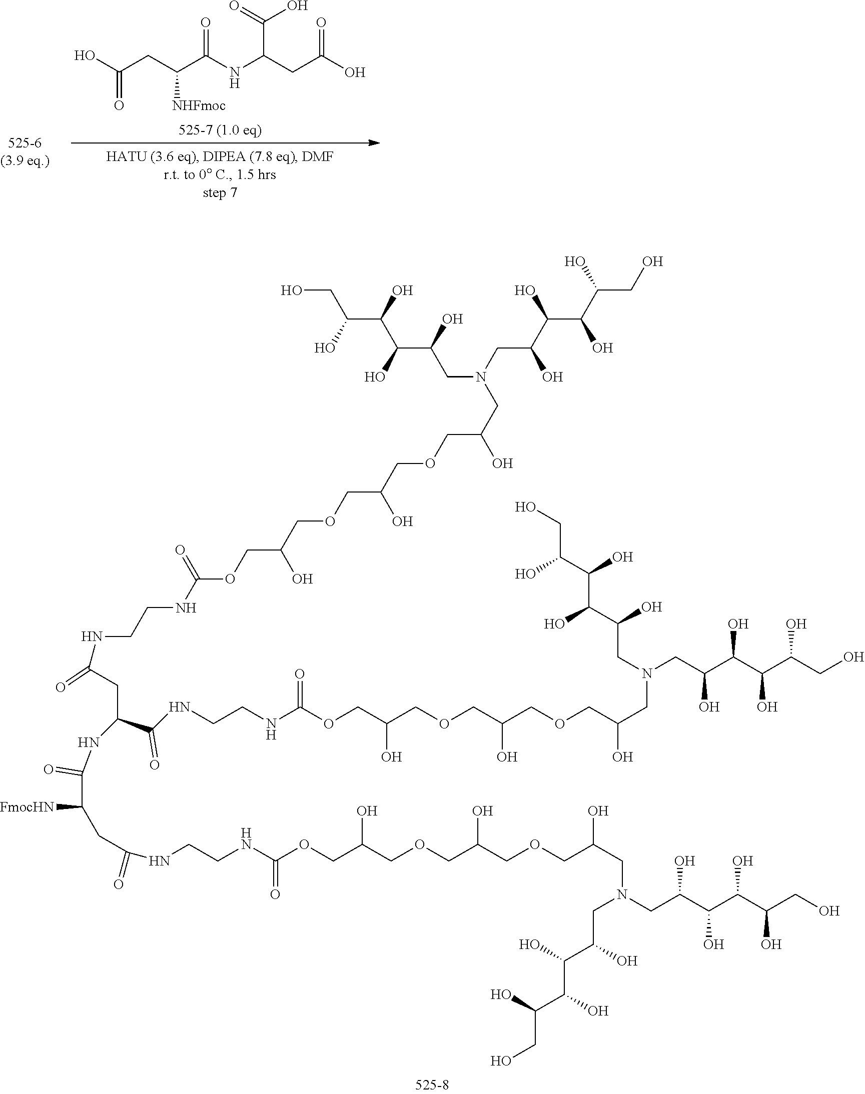

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 1 OH), 4.08-3.87 (m, 12H), 3.84-3.69 (m, 18H), 3.67-3.35 (m, 53H), 3.30-3.07 (m, 12H), 2.77-2.32 (m, 4H).

### Step 8

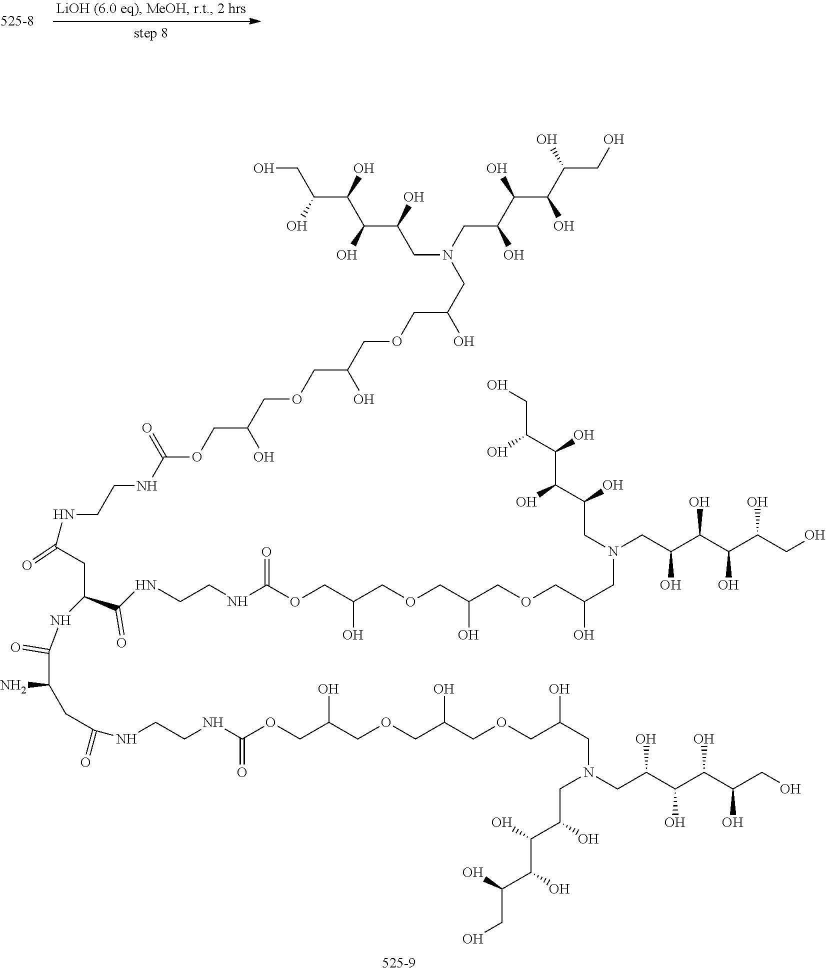 [SMILES: N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

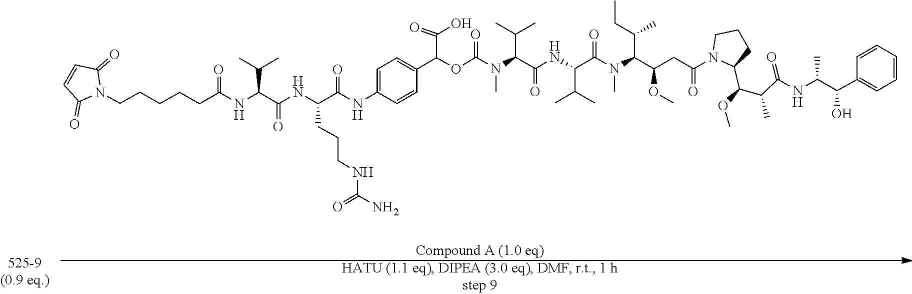 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)O)c1ccc(NC(=O)[C@H](CCCNC(N)=O)NC(=O)[C@@H](NC(=O)CCCCCN2C(=O)C=CC2=O)C(C)C)cc1)C(C)C]

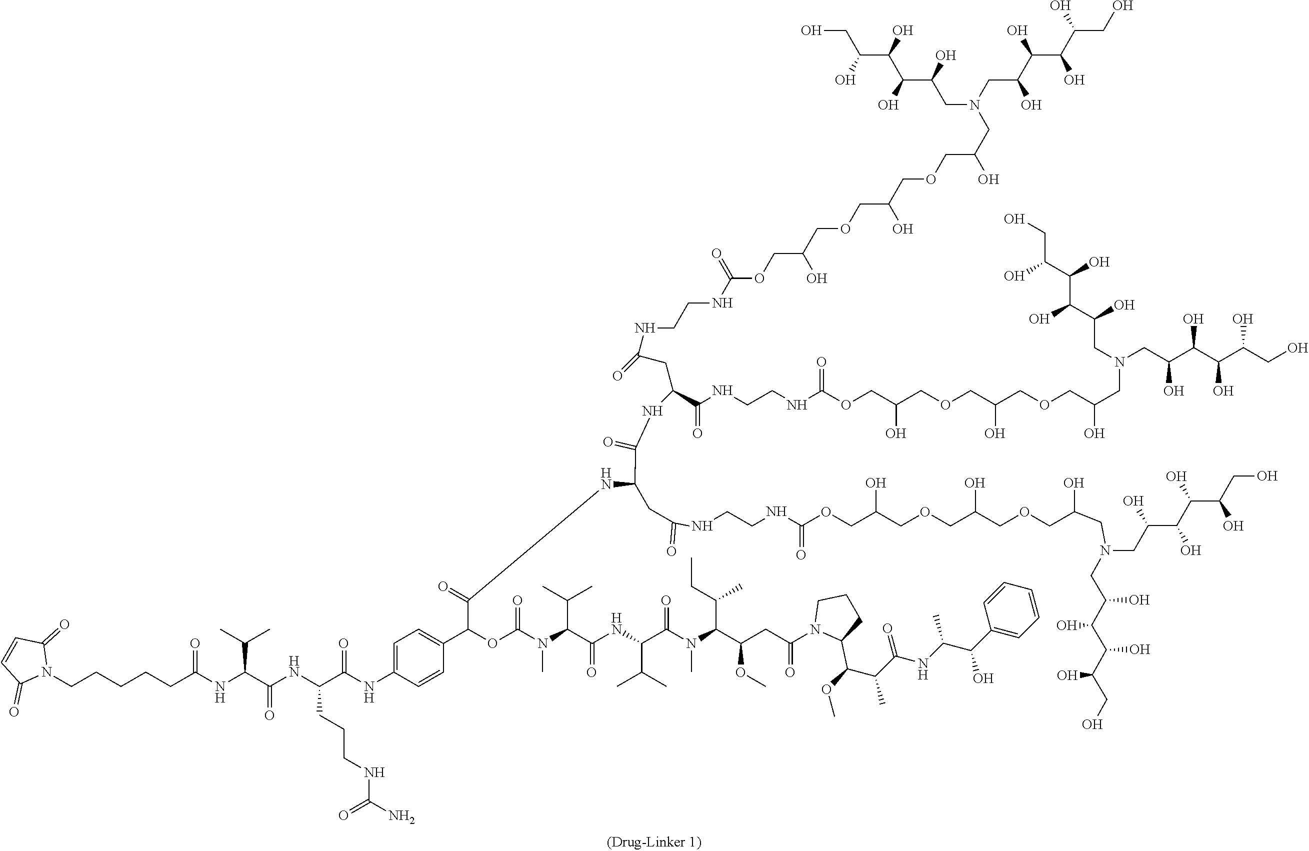

A solution of 525-9 (70.0 mg, 0.0325 mmol), Compound A (49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give drug-linker 1 (23.5 mg, 0.00672 mmol, 20.7%) as a white solid. LCMS, m/z=1749.66 (M/2+H)⁺, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 2: Preparation of Drug-Linker 2

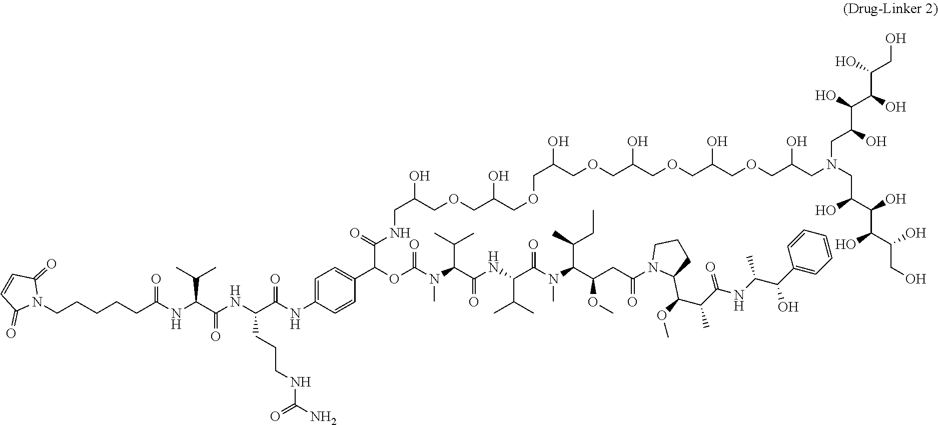 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

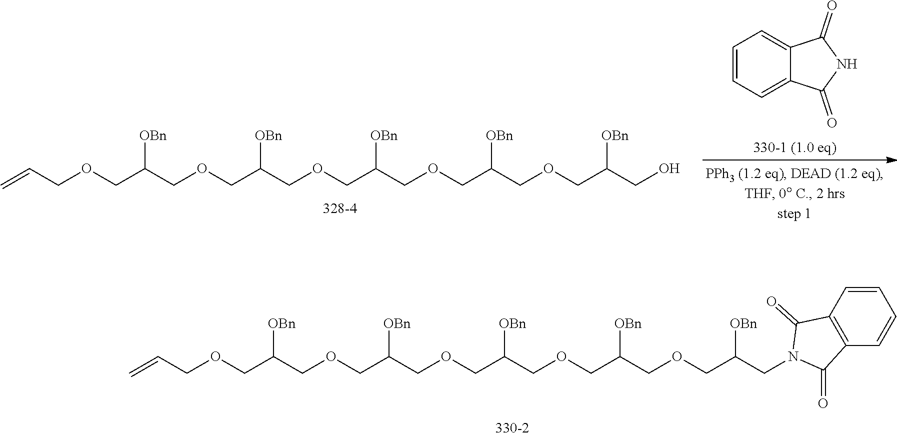 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THE (8 mL) was added a solution of 328-4 (1.3 g, 1.48 mmol) in THE (4 mL) and the mixture was stirred in an ice bath. A solution of DEAD (309.3 mg, 1.776 mmol) in THE (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. ¹H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

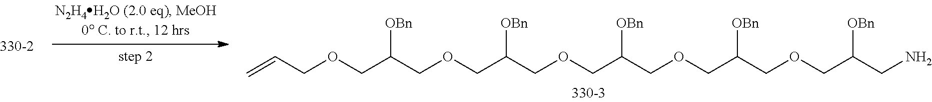 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

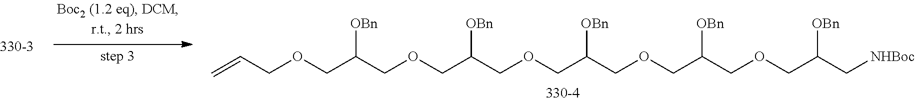 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

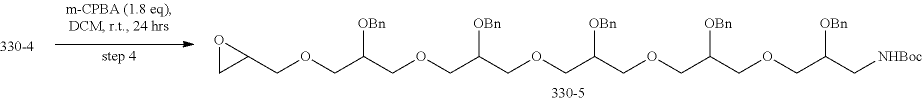 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequencely washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

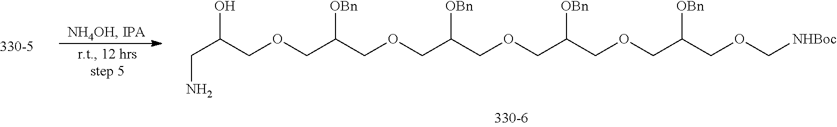 [SMILES: CC(C)(C)OC(=O)NCOCC(COCC(COCC(COCC(COCC(O)CN)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

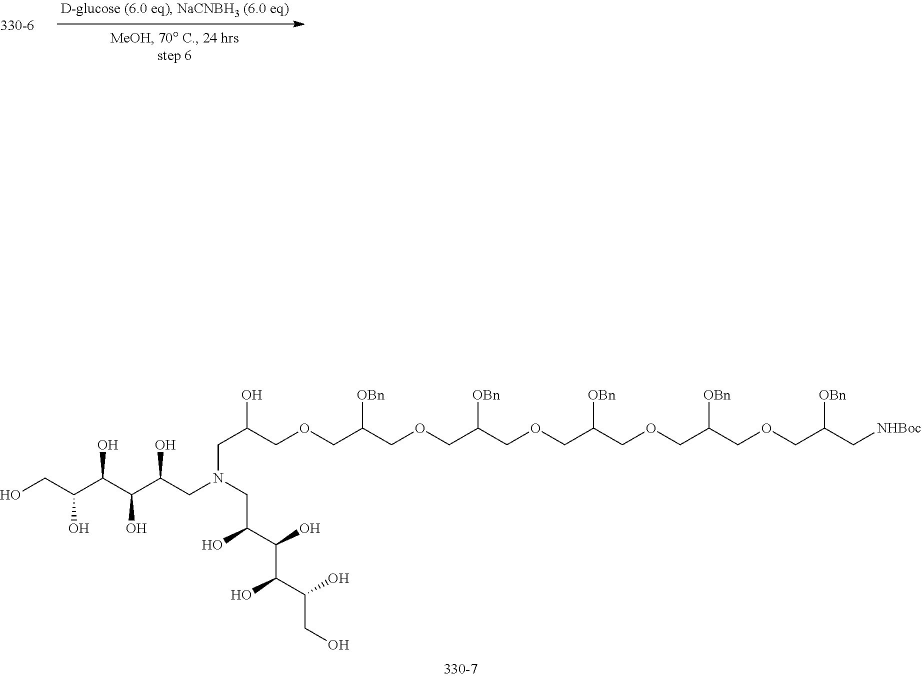 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

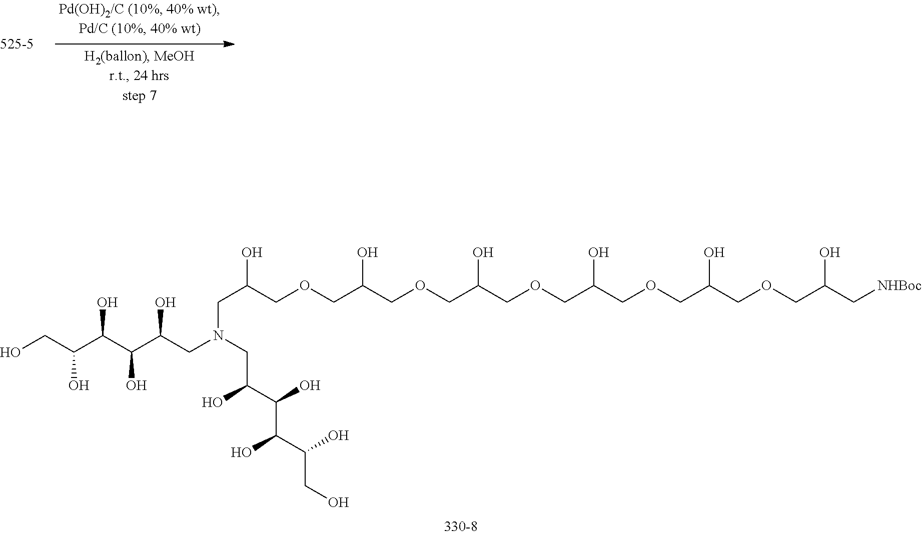 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (ballon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

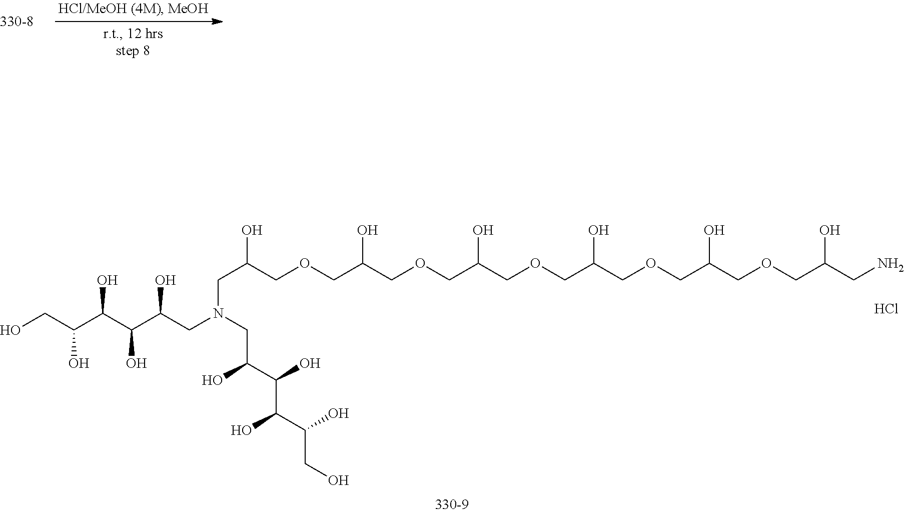 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

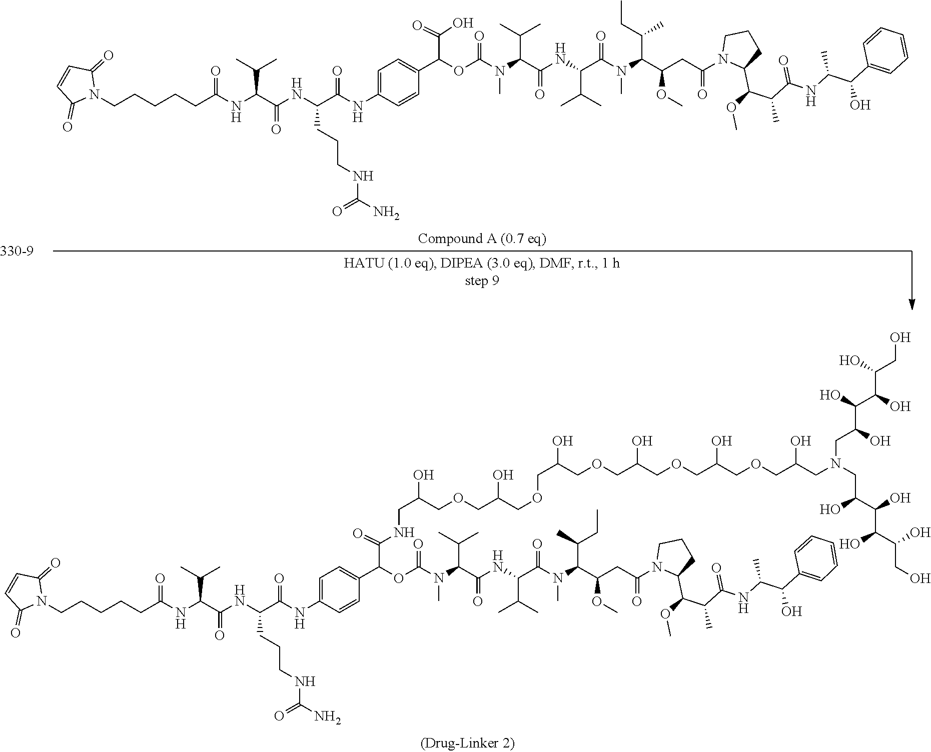

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give drug-linker 2 (28 mg, 0.0131 mmol. 33.7%) as a white solid. LCMS, m/z=1066.11 (M/2+H)⁺. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 3: Preparation of Drug-Linker 3

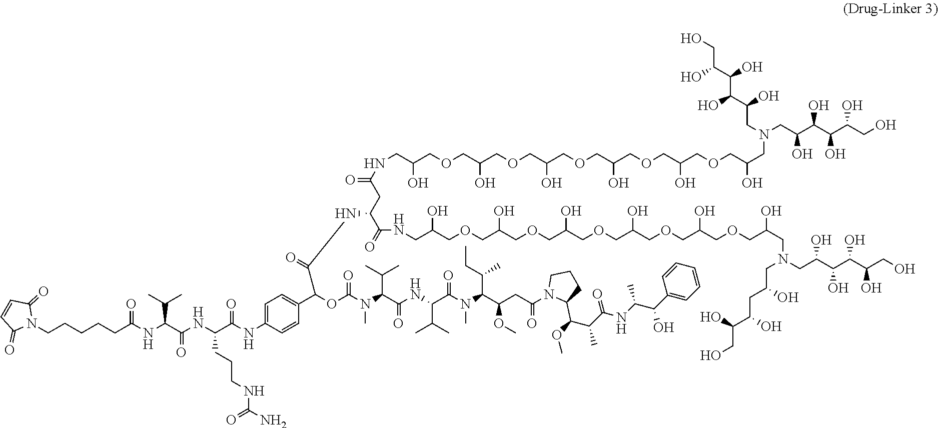 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)C[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

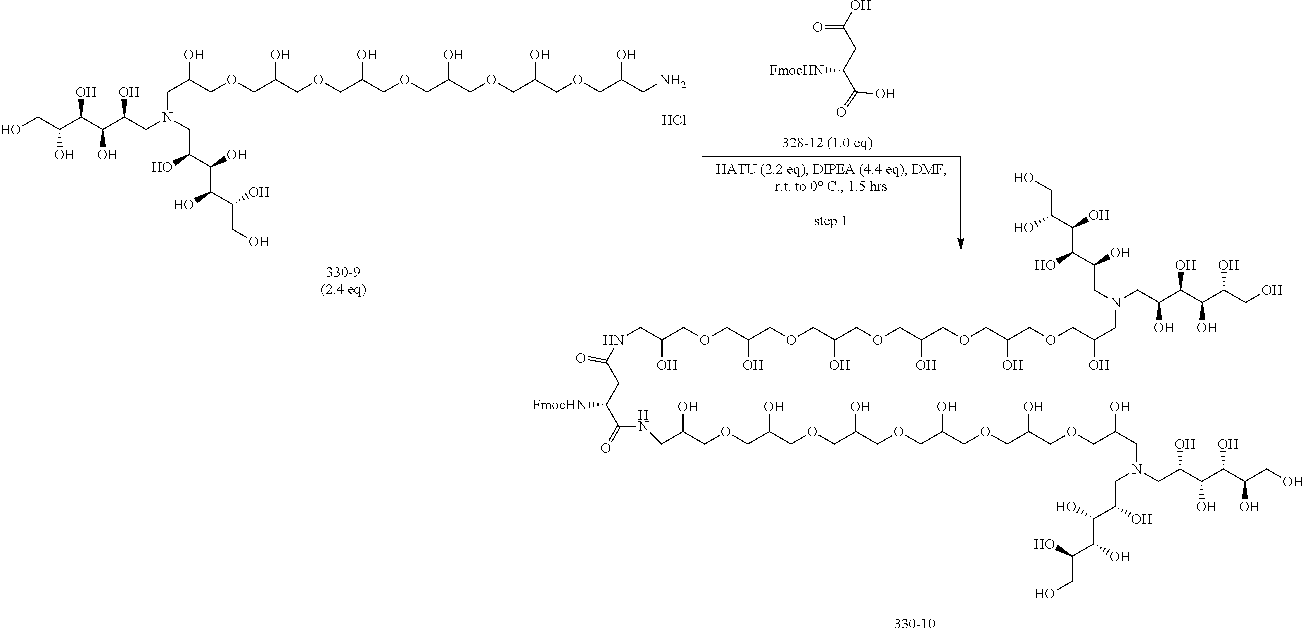

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 2

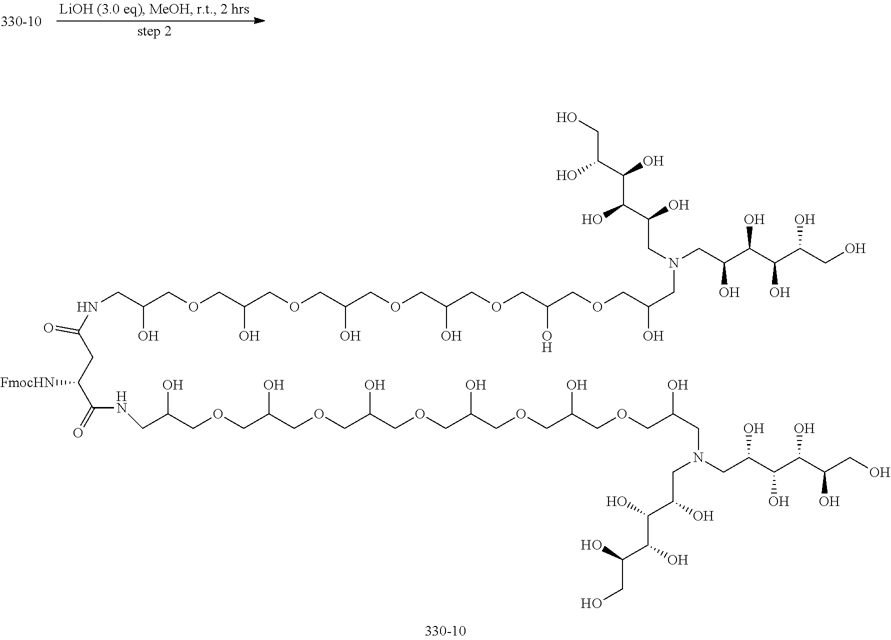

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 3

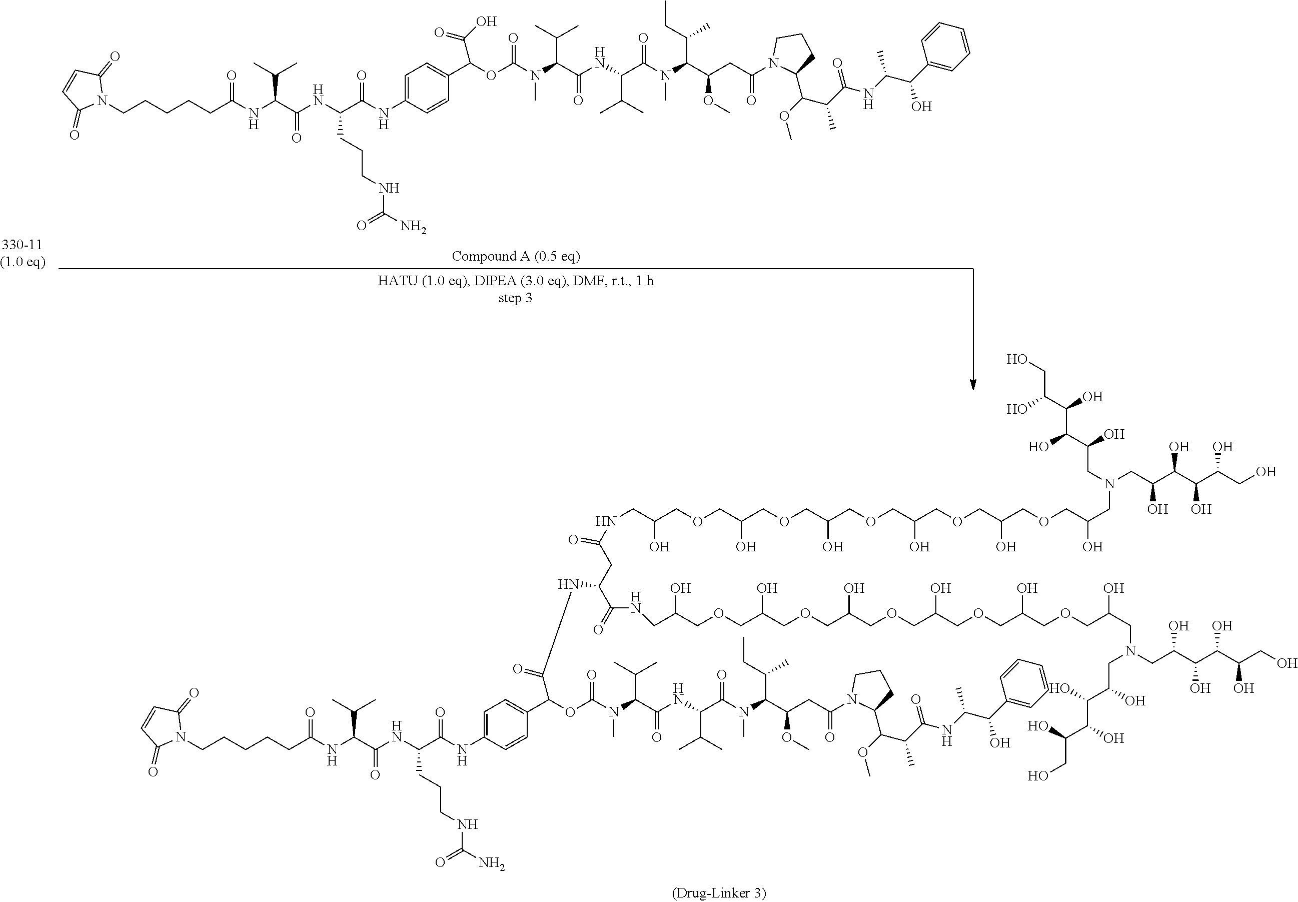

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give drug-linker 3 (5.0 mg, 0.00166 mmol, 45.1%) as a white solid. LCMS, m/z=1509.00 (M/2+H)⁺, m/z=1006.6 (M/3+H)⁺.

### Example 4: Preparation of Drug-Linker 4

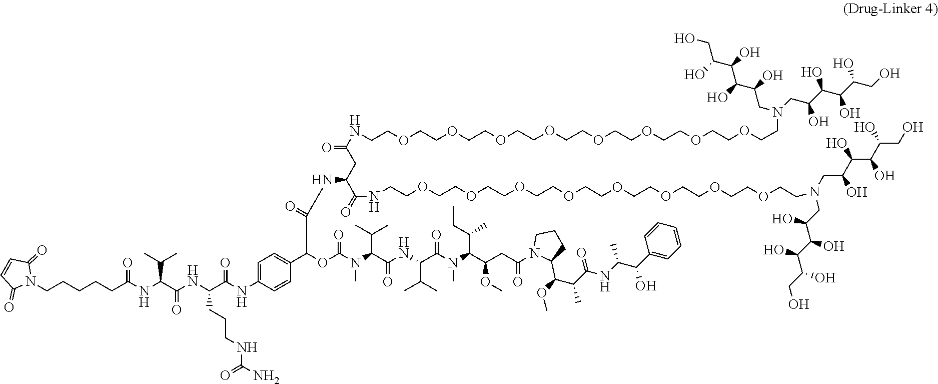 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

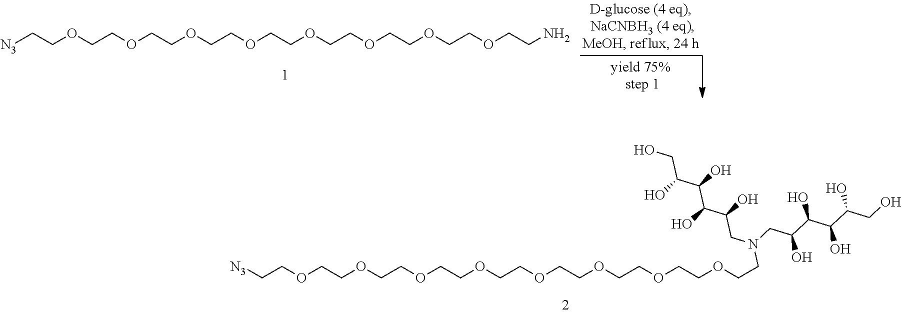 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 1 (2.5 g, 5.701 mmol) in MeOH (20 mL) was added D-Glucose (4.11 g, 22.804 mmol) and NaBH₃CN (1.385 mL, 22.804 mmol). The mixture was stirred at reflux for 24h to complete. Then the resulting solution concentrated to dryness and the residue was purified by reverse phase chromatography (C8 column, eluting with 0-45% methanol in water with 0.01% TFA) to afford the product 2 as yellow oil. ESI m/z: 767.5 (M+H)⁺.

### Step 2

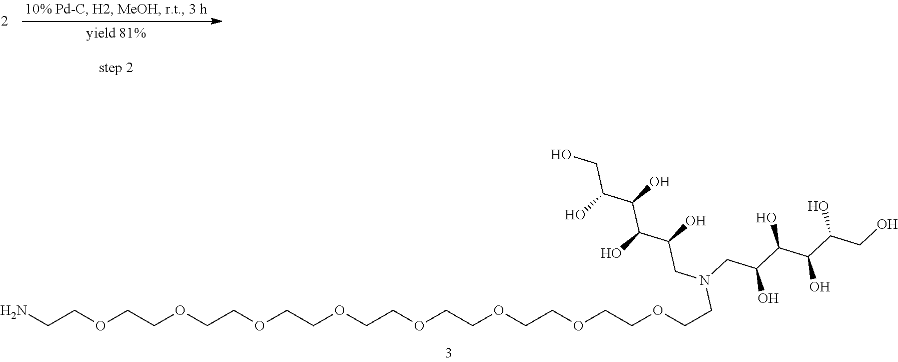 [SMILES: NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3h to complete. The resulting mixture was filtered to remove catalyst solid and the filtrate was concentrated, then purified by reverse phase chromatography (C8 column, eluting with 0-25% acetonitrile in water with 0.01% TFA) to afford the product 3 (2.6 g, 3.510 mmol, 81.50%) as colorless oil. ESI m/z: 371.3 (M/2+H)+, 741.4 (M+H)⁺.

### Step 3

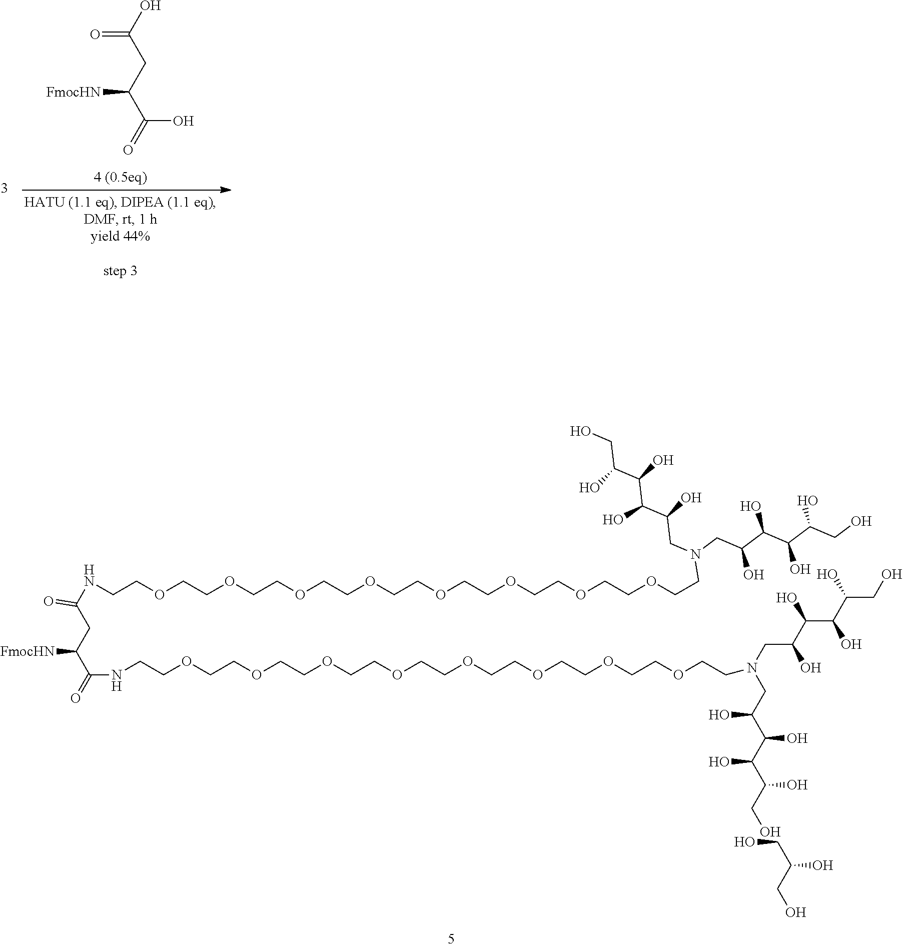

A solution of compound 5 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 3 (2.6 g, 3.510 mmol) in DMF (5 mL). After addition, the solution was stirred at room temperature for another 1 h to complete. The completed solution was then purified directly by reverse phase chromatography (C8 column, eluting with 0-40% acetonitrile in water with 0.01% TFA) to afford the product 5 (1.4 g, 0.777 mmol, 44.30%) as colorless oil. ESI m/z: 601.0 (M/3+H)⁺, 901.0 (M/2+H)⁺.

### Step 4

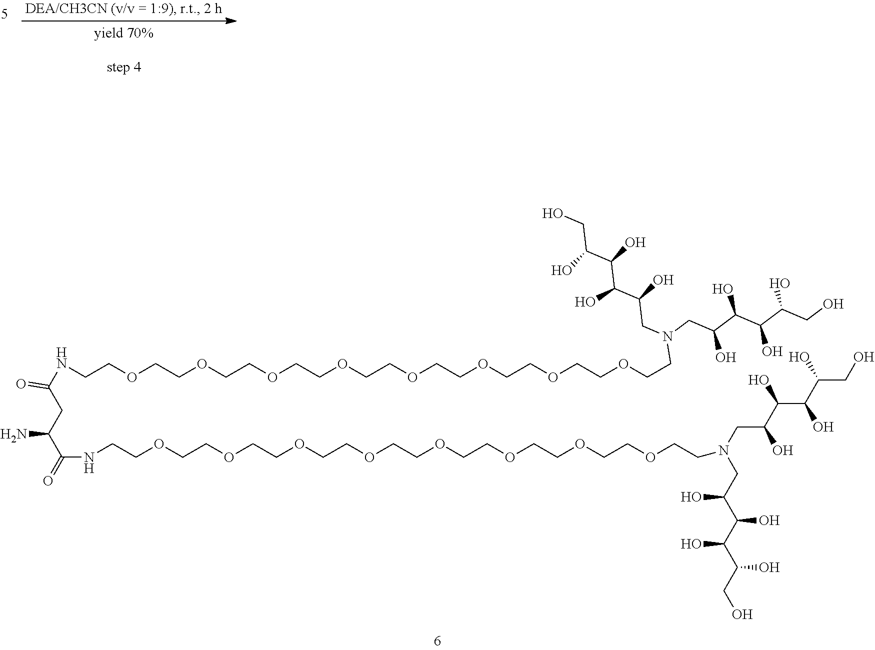 [SMILES: N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 5 (1.4 g, 0.777 mmol) in MeCN (6 mL) was diethyl amine (0.7 mL, 8.930 mmol). The mixture was stirred at room temperature for 2h to achieve complete deprotection. Then the resulting solution was concentrated under reduced pressure to remove most of diethyl amine, and the residue was purified by reverse phase chromatography (C8 column, eluting with 0-20% acetonitrile in water with 0.01% TFA) to get desired fractions, which was freeze-dried to afford 6 as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

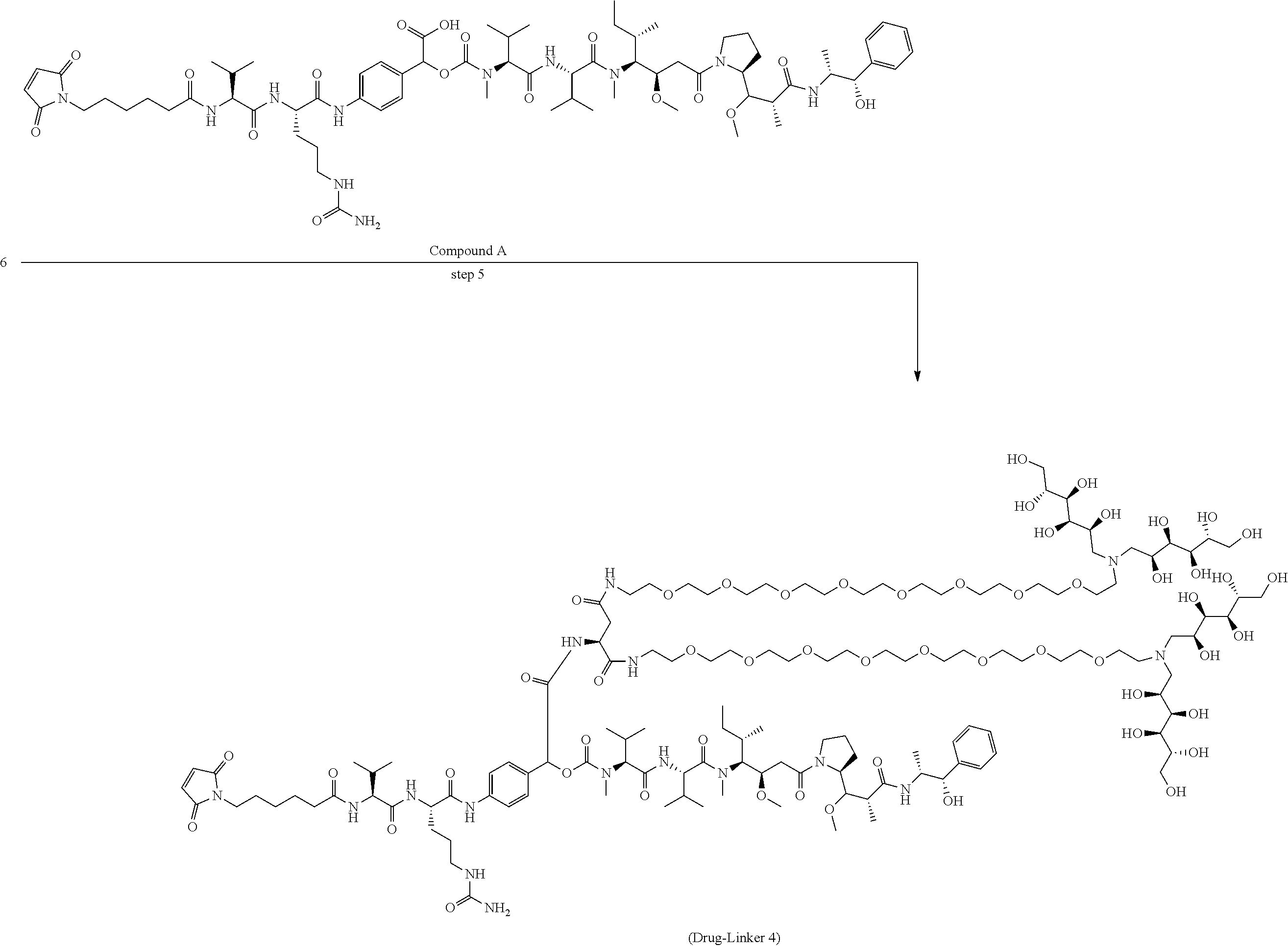

A solution of 6 (100 mg, 0.063 mmol), Compound A (86 mg, 0.063 mmol) and HATU (24 mg, 0.063 mmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give drug-linker 4 (40 mg, 0.014 mmol, 21.73%) as a white solid. LCMS, m/z=1461.23 (M/2+H)⁺; ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 10H), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 10OH), 1.24-1.08 (m, 9H), 1.02-1.01 (m, 2H), 0.92-0.75 (m, 28H) ppm.

### Example 5: Preparation of Drug-Linker 5

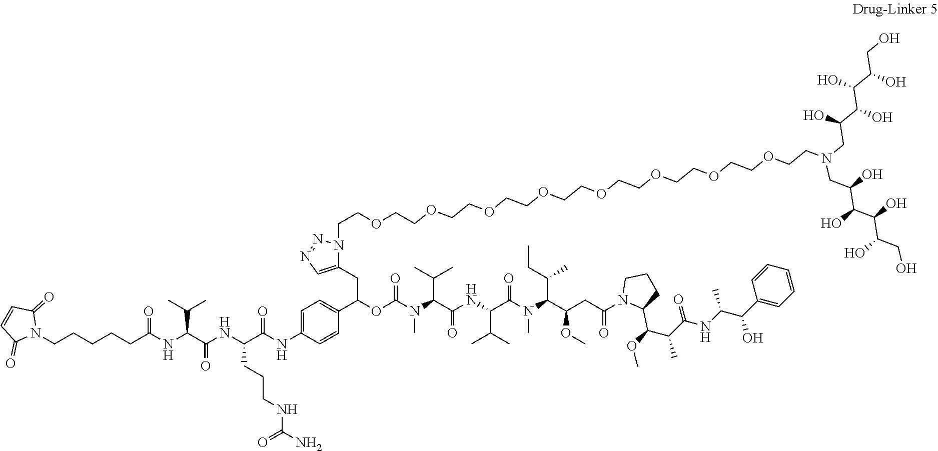 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

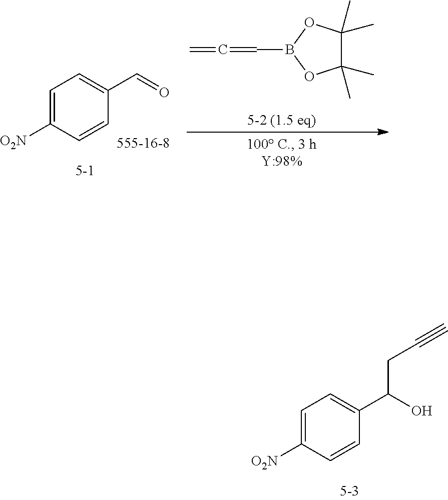

The mixture of 4-nitrobenzaldehyde 5-1 (10 g, 66.173 mmol) and 4,4,5,5-tetramethyl-2-(propa-1,2-dien-1-yl)-1,3,2-dioxaborolane 5-2 (17.532 mL, 99.259 mmol) was heated at 100° C. under nitrogen atmosphere with stirring for 3h to achieve complete conversion. Then the resulting solution was purified by flash chromatography (silica gel, eluting with 0-30% EA in PE) to afford the product 5-3 (12.6 g, 65.903 mmol, 99.60%) as a pale yellow solid. ESI m/z 192.1 (M+H)⁺.

### Step 2

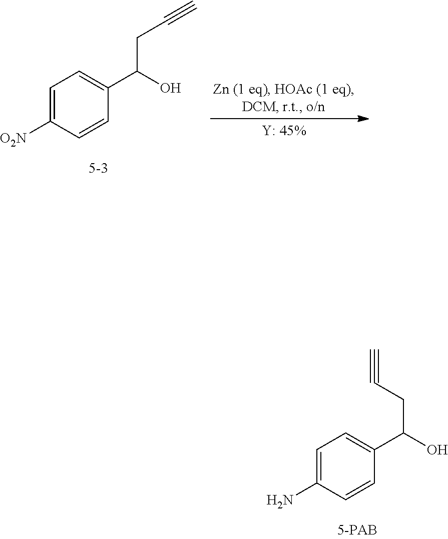

To the solution of 1-(4-nitrophenyl)but-3-yn-1-ol 5-3 (16 g, 83.686 mmol) in DCM (30 mL) was added Zn powder (0.767 mL, 83.686 mmol) and acetic acid (4.795 mL, 83.686 mmol). Then the mixture was stirred at room temperature for overnight. After completion, the mixture was filtered to remove the zinc solid, and the filtrate was concentrated and purified by reverse phase flash chromatography (C18 column, eluting with 0-15% acetonitrile in water with 0.01% TFA) to afford the product 1-(4-aminophenyl)but-3-yn-1-ol 5-PAB (6.1 g, 37.841 mmol, 45.22%) as a brown solid. ESI m/z: 162.2 (M+H)⁺.

### Step 3

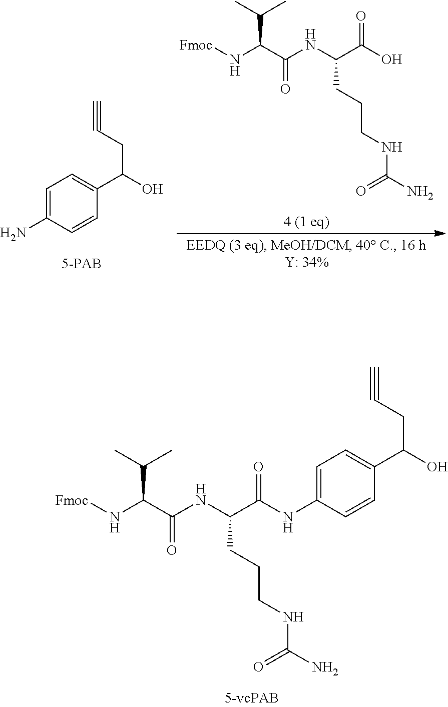

To the solution of (2S)-5-(carbamoylamino)-2-[(2S)-2-({[(9H-fluoren-9-yl)methoxy]carbonyl}amino)-3-methylbutanamido]pentanoic acid 4 (2.5 g, 5.035 mmol) in MeOH (8 mL) and DCM (32 mL) was added 1-(4-aminophenyl)but-3-yn-1-ol 5-PAB (0.81 g, 5.035 mmol) and EEDQ (3.74 g, 15.104 mmol). The mixture was stirred at 40° C. for overnight to complete. The resulting solution was concentrated to dryness and then purified by flash chromatography (silica gel, eluting with 0-10% methanol in DCM) to afford the product (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol, 32.30%) as a pale yellow solid. ESI m/z: 640.3 (M+H)⁺. ¹H NMR (400 MHz, DMSO-d6) δ 9.99 (s, 1H), 8.10 (d, J=7.6 Hz, 1H), 7.88 (d, J=7.6 Hz, 2H), 7.76-7.72 (m, 2H), 7.53 (d, J=8.8 Hz, 2H), 7.45-7.39 (m, 3H), 7.32-7.26 (m, 3H), 5.96 (m, 1H), 5.44-5.39 (m, 3H), 4.62 (t, J=6.0 Hz, 1H), 4.42-4.40 (m, 1H), 4.31-4.20 (m, 3H), 3.95-3.90 (m, 1H), 3.00-2.91 (m, 2H), 2.70-2.69 (m, 1H), 2.45-2.32 (m, 2H), 2.00-1.97 (m, 1H), 1.59-1.23 (m, 5H), 0.88-0.83 (m, 6H) ppm.

### Step 4

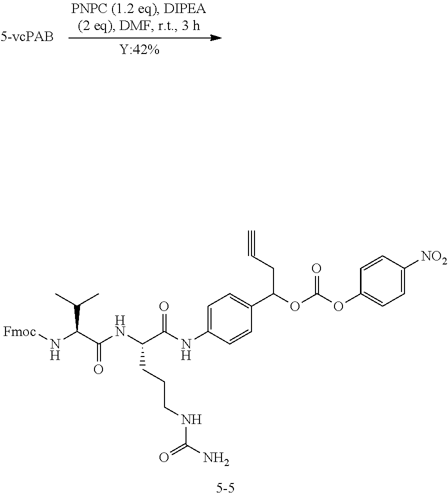

To the solution of (9H-fluoren-9-yl)methyl N-[(1S)-1-{[(1S)-4-(carbamoylamino)-1-{[4-(1-hydroxybut-3-yn-1-yl)phenyl]carbamoyl}butyl]carbamoyl}-2-methylpropyl]carbamate 5-vcPAB (1.04 g, 1.626 mmol) and DIPEA (0.42 g, 3.252 mmol) in DMF (15 mL) was added bis(4-nitrophenyl) carbonate (PNPC, 0.59 g, 1.951 mmol) portionwise. After addition, the mixture was stirred at room temperature for another 3 h. After completion according to LCMS monitoring, the resulting solution was purified directly by reverse phase flash chromatography (C18 column, eluting with 0-75% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methyl butanamido]pentanamido]phenyl}but-3-yn-1-yl 4-nitrophenyl carbonate 5-5 (560 mg, 0.696 mmol, 42.75%) as a pink solid. ESI m/z: 805.3 (M+H)⁺.

### Step 5

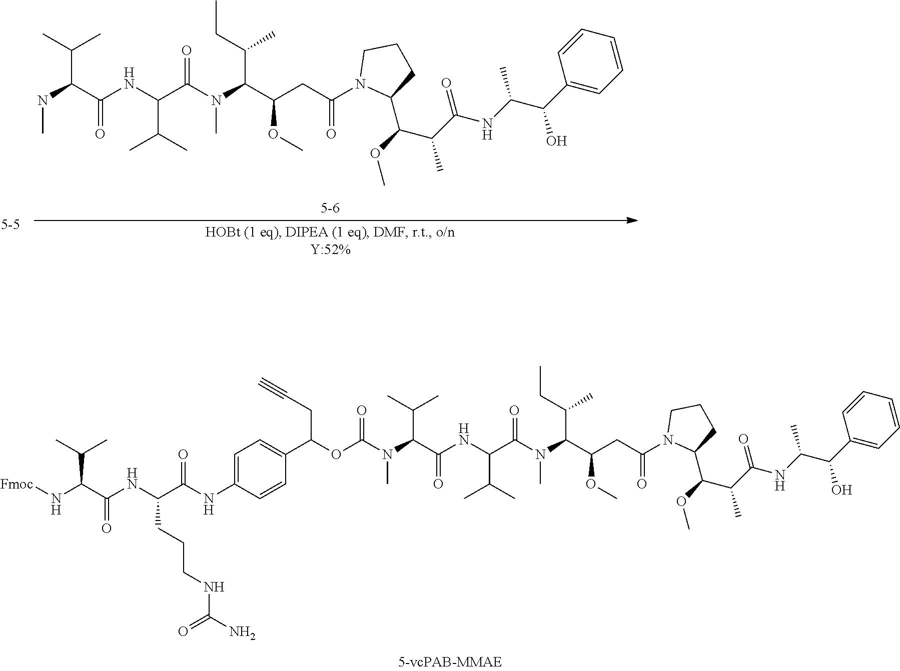

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl 4-nitrophenyl carbonate 5-5 (540 mg, 0.671 mmol) in DMF (15 mL) was added (2S)—N-[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]-3-methyl-2-(methylamino)butanamide 5-6 (481.72 mg, 0.671 mmol), HOBt (90.66 mg, 0.671 mmol) and DIPEA (86.71 mg, 0.671 mmol). The mixture was stirred at room temperature for overnight to achieve complete reaction. Then the resulting solution was purified directly by reverse phase flash chromatography (C8 column, eluting with 0-70% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (486 mg, 0.351 mmol, 52.34%) as a white solid. ESI m/z: 692.3 (M/2+H)⁺.

### Step 6

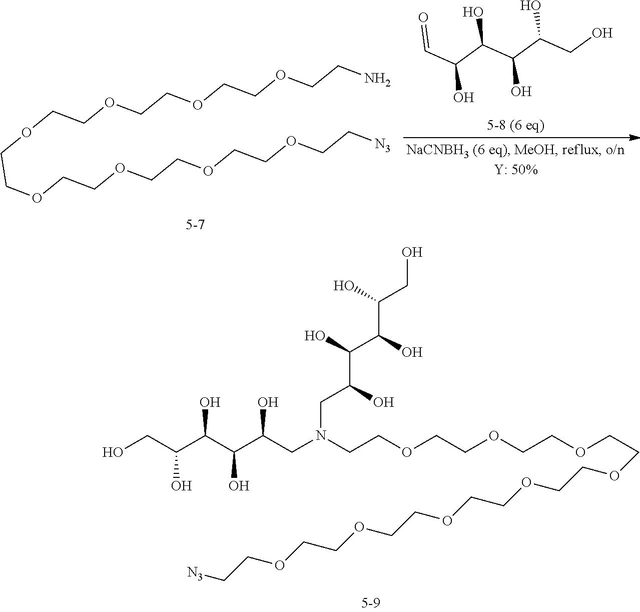 [SMILES: [N-]=[N+]=NCCOCCOCCOCCOCCOCCOCCOCCOCCN ; O=C[C@H](O)[C@@H](O)[C@H](O)[C@H](O)CO ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 26-azido-3,6,9,12,15,18,21,24-octaoxahexacosan-1-amine 5-7 (160 mg, 0.365 mmol) and D-glucose 5-8 (394.40 mg, 2.189 mmol) in MeOH (6 mL) was treated with NaBH₃CN (0.133 mL, 2.189 mmol). Then the mixture was stirred at 75° C. for overnight. Then the complete reaction solution was concentrated to dryness and the residue was purified by reverse phase flash chromatography (C8 column, eluting with 0-10% acetonitrile in water with 0.01% TFA) to afford the product (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (140 mg, 50%) as transparent oil. ESI m/z: 767.3 (M+H)⁺.

### Step 7

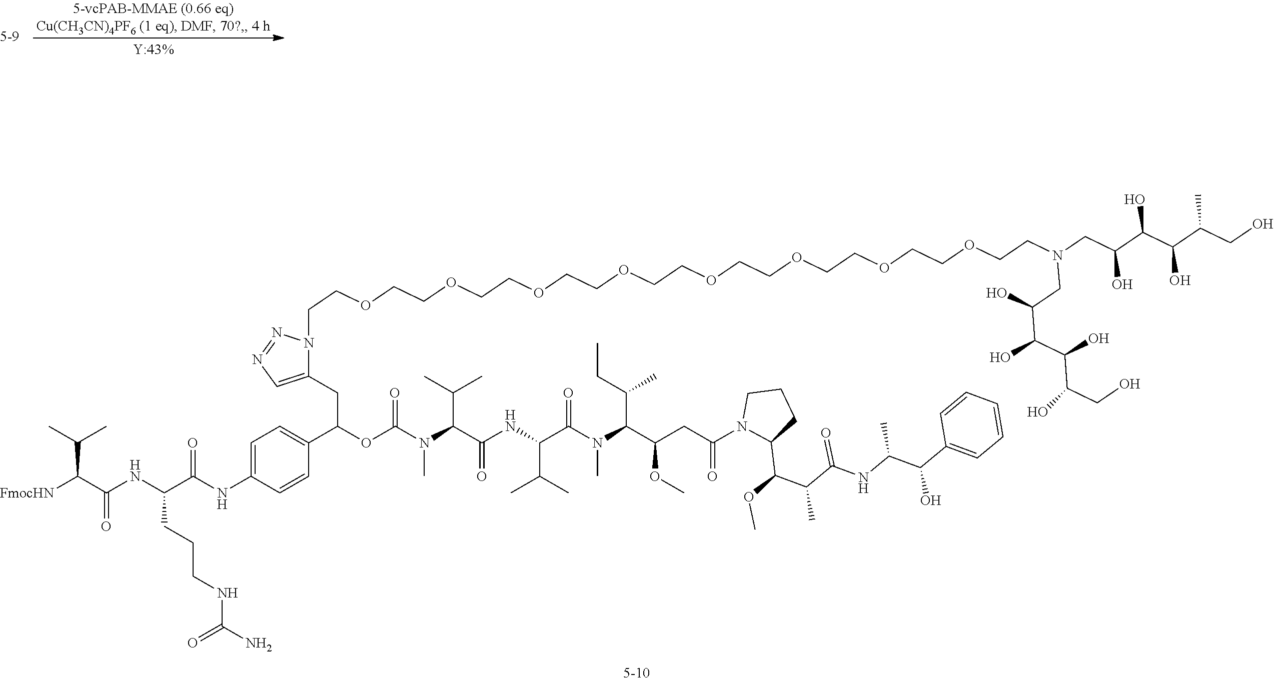

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}but-3-yn-1-yl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-vcPAB-MMAE (300 mg, 0.217 mmol) in DMF (6 mL) was added (29S,30R,31R,32R)-1-azido-27-((2S,3R,4R,5R)-2,3,4,5,6-pentahydroxyhexyl)-3,6,9,12,15,18,21,24-octaoxa-27-azatritriacontane-29,30,31,32,33-pentaol 5-9 (249.38 mg, 0.325 mmol) and Cu(CH₃CN)₄PF₆ (121.55 mg, 0.325 mmol) at room temperature. The resulting mixture was heated at 70° C. with stirring for 4 h. The completed reaction solution was then purified by reverse phase flash chromatography (C8 column, eluting with 0-37% acetonitrile in water with 0.01% TFA) to afford the product 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (204 mg, 0.095 mmol, 43.71%) as a white solid. ESI m/z: 1075.6 (M/2+H)⁺.

### Step 8

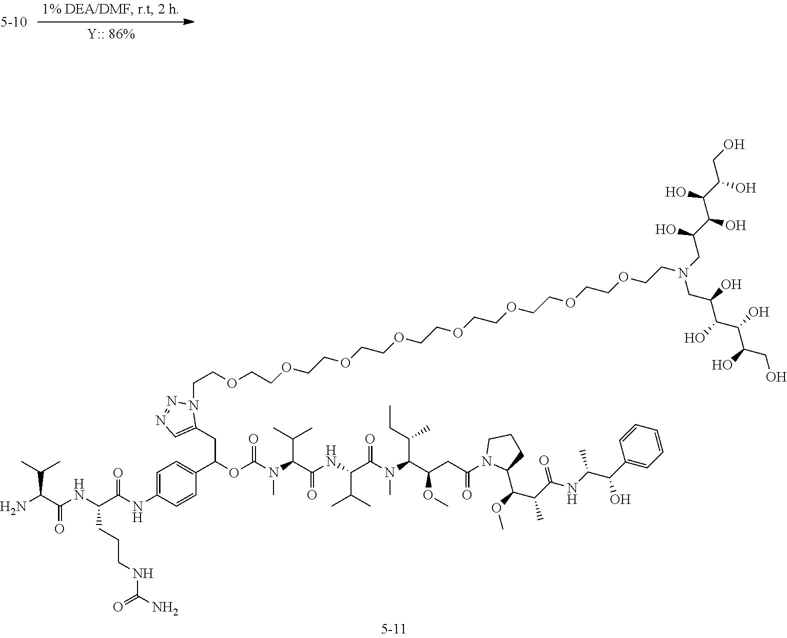 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(Cc1cnnn1CCOCCOCCOCCOCCOCCOCCOCCOCCN(C[C@@H](O)[C@H](O)[C@@H](O)[C@@H](O)CO)C[C@@H](O)[C@@H](O)[C@H](O)[C@H](O)CO)c1ccc(NC(=O)[C@H](CCCNC(N)=O)NC(=O)[C@@H](N)C(C)C)cc1)C(C)C]

To the solution of 1-{4-[(2S)-5-(carbamoylamino)-2-[(2S)-2-({[(9H-fluoren-9-yl)methoxy]carbonyl}amino)-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-10 (167 mg, 0.078 mmol) in DMF (2 mL) was added diethylamine (DEA, 0.02 mL, 0.125 mmol). The mixture was stirred at room temperature for 2h to complete. The resulting solution was purified by reverse phase flash chromatography (C8 column, eluting with 0-40% acetonitrile in water with 0.01% TFA) to afford the product 1-{4-[(2S)-2-[(2S)-2-amino-3-methylbutanamido]-5-(carbamoylamino)pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol, 86.81%) as a white solid. ESI m/z: 965.5 (M/2+H)⁺.

### Step 9

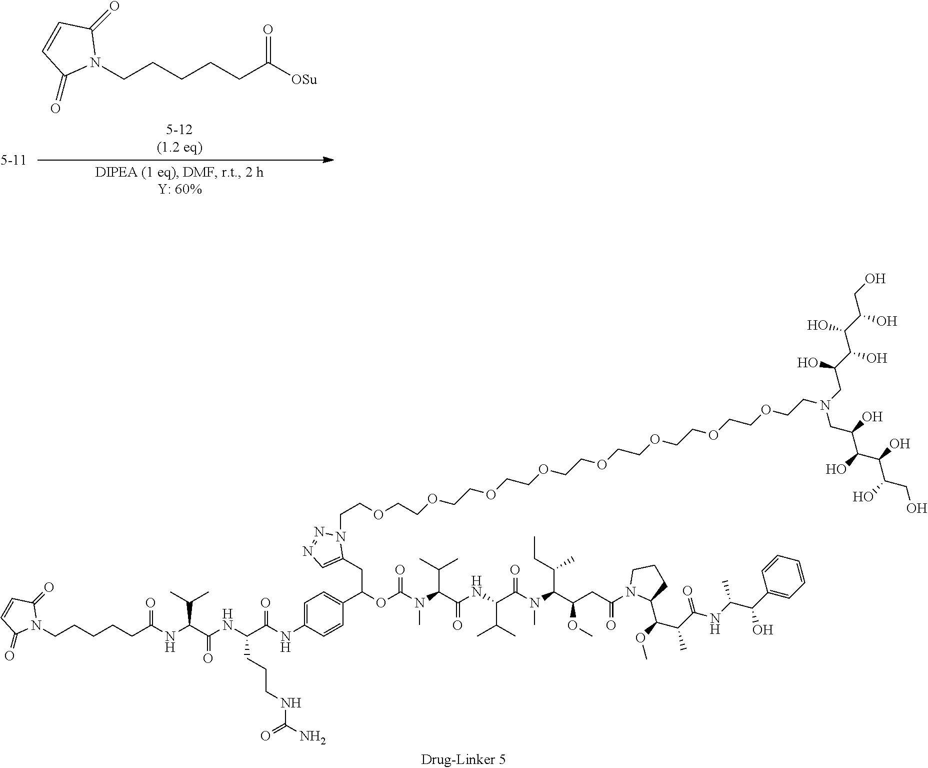

To the solution of 1-{4-[(2S)-2-[(2S)-2-amino-3-methylbutanamido]-5-(carbamoylamino)pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate 5-11 (130 mg, 0.067 mmol) in DMF (3 mL) was added 2,5-dioxopyrrolidin-1-yl 6-(2,5-dioxo-2,5-dihydro-1H-pyrrol-1-yl)hexanoate 5-12 (24.79 mg, 0.080 mmol) and DIPEA (8.66 mg, 0.067 mmol) sequentially. The resulting solution was stirred at room temperature for 2h to complete full conversion. The reaction solution was then purified directly by reverse phase flash chromatography (C8 column, eluting with 0-40% acetonitrile in water with 001% TFA) to afford the product 1-{4-[(2S)-5-(carbamoylamino)-2-[(2S)-2-[6-(2,5-dioxo-2,5-dihydro-1H-pyrrol-1-yl)hexanamido]-3-methylbutanamido]pentanamido]phenyl}-2-{1-[(29S,30R,31R,32R)-29,30,31,32,33-pentahydroxy-27-[(2S,3R,4R,5R)-2,3,4,5,6-pentahydroxyhexyl]-3,6,9,12,15,18,21,24-octaoxa-27-azatritriacontan-1-yl]-1H-1,2,3-triazol-5-yl}ethyl N-[(1S)-1-{[(1S)-1-{[(3S,4S,5S)-1-[(2S)-2-[(1R,2R)-2-{[(1R,2S)-1-hydroxy-1-phenylpropan-2-yl]carbamoyl}-1-methoxy-2-methylethyl]pyrrolidin-1-yl]-3-methoxy-5-methyl-1-oxoheptan-4-yl](methyl)carbamoyl}-2-methylpropyl]carbamoyl}-2-methylpropyl]-N-methylcarbamate (86 mg, 0.041 mmol, 60.13%) drug-linker 5 as a white solid. ESI m/z: 1061.8 (M/2+H)⁺, retention time 6.062 min (HPLC). ¹H NMR (400 MHz, DMSO-d6) δ 9.95 (s, 1H), 8.60-8.45 (m, 1H), 8.20-8.06 (m, 2H), 7.95-7.90 (m, 1H), 7.83-7.80 (m, 1H), 7.72-7.60 (m, 1.5H), 7.54-7.50 (m, 2H), 7.43-7.40 (m, 1H), 7.31-7.12 (m, 6H), 6.99 (s, 2H), 6.03 (brs, 1H), 5.82-5.68 (m, 1.5H), 5.50-5.30 (m, 3H), 4.76-4.52 (m, 3H), 4.50-4.30 (m, 8H), 4.29-4.11 (m, 3H), 4.00-3.92 (m, 8H), 3.77-3.66 (m, 16H), 3.66-3.48 (m, 18H), 3.45-3.29 (m, 12H), 3.24-3.07 (m, 12H), 3.00-2.68 (m, 8H), 2.44-1.94 (m, 7H), 1.80-1.67 (m, 4H), 1.58-1.34 (m, 10H), 1.21-1.15 (m, 2H), 1.05-0.96 (m, 7H), 0.90-0.50 (m, 28H) ppm.

### Example 6: Preparation of Drug-Linker 6

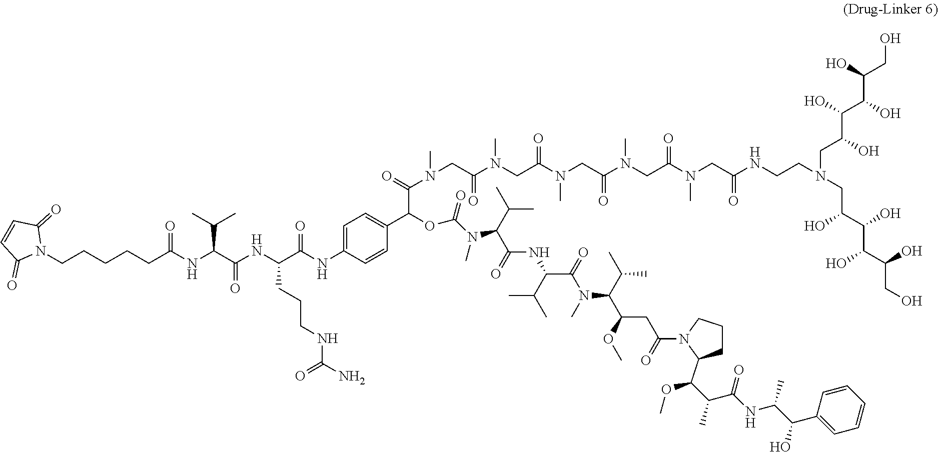

### Step 1

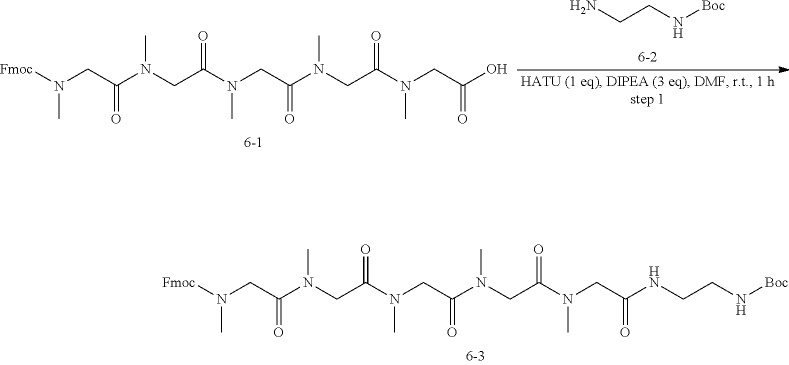

A solution of 6-1 (300 mg, 0.504 mmol), 6-2 (81 mg, 0.504 mmol) and HATU (192 mg, 0.504 mmol) in anhydrous DMF (10 mL) was stirred at room temperature for 5 min, then DIPEA (196 mg, 1.512 mmol) was added. The resulting solution was stirred for another 1 hr at r.t. until LCMS indicated complete reaction. The reaction solution was purified directly by reverse phase liquid chromatography to give 6-3 (227 mg, 0.308 mmol, 61.19%) as a white solid.

### Step 2

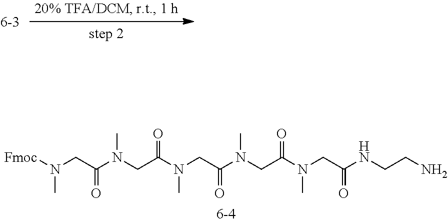

A solution of 6-3 (227 mg, 0.308 mmol) and TFA (2 mL) in anhydrous DCM (8 mL) was stirred at room temperature for 1 hr until LCMS indicated all starting amine was disappeared and desired product was detected. Then the solution was concentrated to dryness to 6-4 (185 mg, 0.290 mmol, 94.18%) as yellow oil, used as such in the next step.

### Step 3

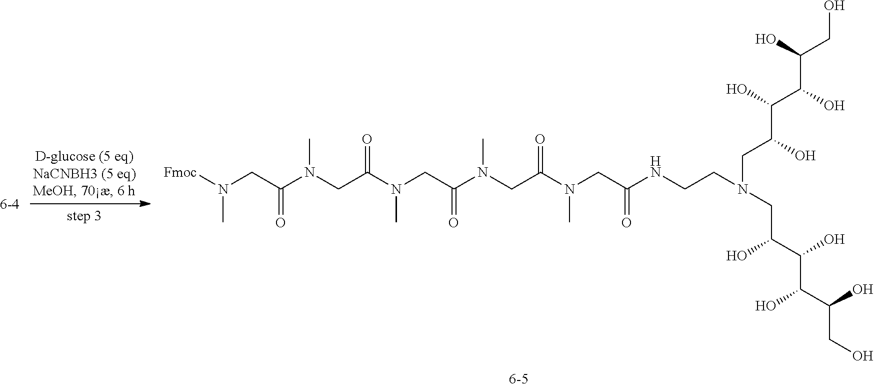

A solution of 6-4 (185 mg, 0.290 mmol) and D-Glucose (261 mg, 1.450 mmol) in anhydrous Methanol (50 mL) was heated at 50° C. for 30 min, then NaCNBH₃ (92 mg, 1.450 mmol) was added. The resulting solution was stirred for another 6 hr at 70° C. until indicated all starting amine was disappeared and the mass of desired product was detected. Then the reaction solution was concentrated and purified by reverse phase liquid chromatography to give 6-5 (125 mg, 0.129 mmol, 44.48%) as a white solid.

### Step 4

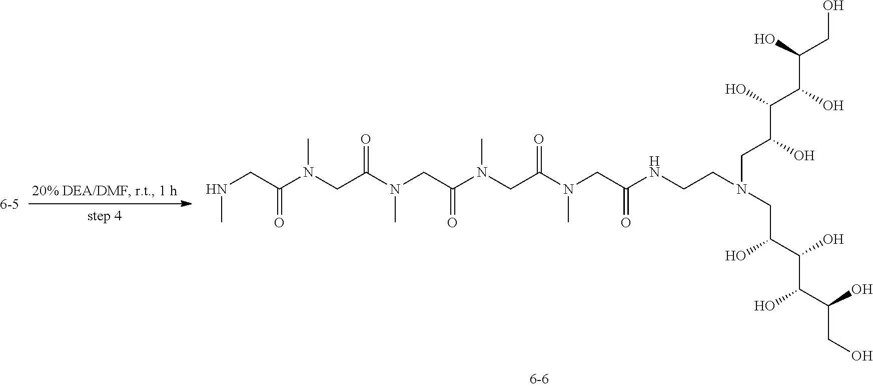 [SMILES: CNCC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 6-5 (125 mg, 0.129 mmol) and DEA (1 mL) in anhydrous DMF (4 mL) was stirred at room temperature for 1 hr until LCMS indicated all starting amine was disappeared and desired product was detected. Then the solution was concentrated to dryness to 6-6 (91 mg, 0.122 mmol, 94.57%) as colorless oil, used as such in the next step.

### Step 5

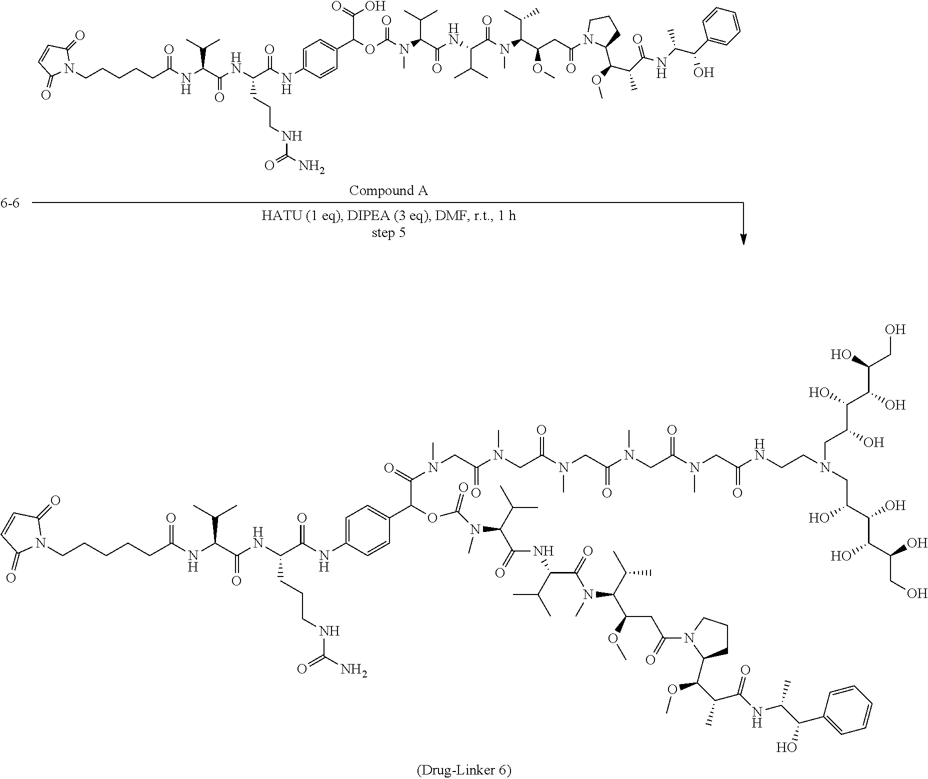

A solution of 6-6 (12 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give drug-linker 6 (12 mg, 0.006 mmol, 38.35%) as a white solid. LCMS, m/z=1044.15 (M/2+H)⁺; ¹H NMR (400 MHz, D₂O) δ 7.46-7.44 (m, 4H), 7.27-7.21 (m, 7H), 4.35-4.26 (m, 9H), 4.14-3.96 (m, 10H), 3.79-3.62 (m, 10H), 7.00 (s, 2H), 3.36-3.10 (m, 16H), 3.05-2.79 (m, 28H), 2.67-2.55 (m, 7H), 2.42-2.17 (m, 5H), 1.80-1.66 (m, 5H), 1.53-1.40 (m, 1OH), 1.2-0.63 (m, 37H).

### Example 7: Preparation of Drug-Linker 7

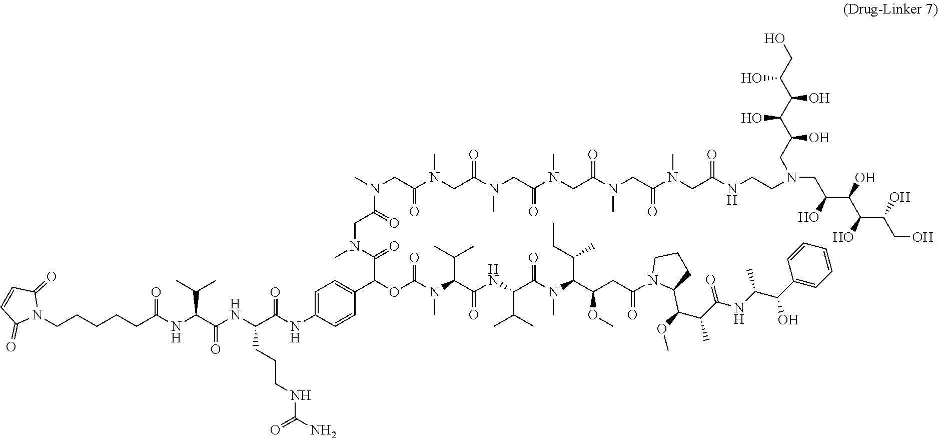 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

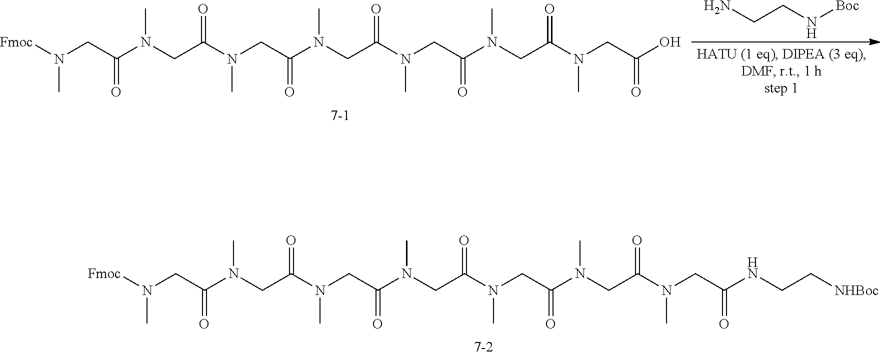

A solution of 7-1 (300 mg, 0.407 mmol), tert-butyl (2-aminoethyl)carbamate (65 mg, 0.407 mmol) and HATU (155 mg, 0.407 mmol) in anhydrous DMF (10 mL) was stirred at room temperature for 5 min, then DIPEA (158 mg, 1.221 mmol) was added.

The resulting solution was stirred for another 1 hr at r.t. until LCMS indicated complete reaction. The reaction solution was purified directly by reverse phase liquid chromatography to give 7-2 (243 mg, 0.276 mmol, 67.81%) as a white solid.

### Step 2

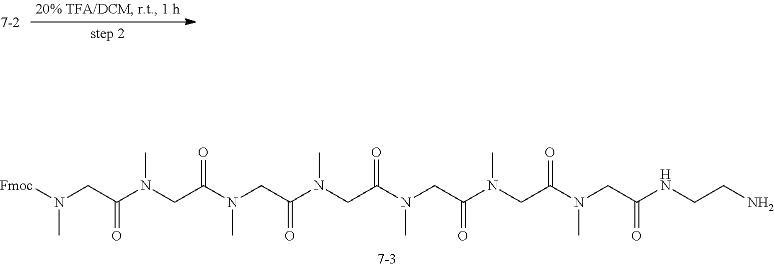

A solution of 7-2 (243 mg, 0.276 mmol) and TFA (2 mL) in anhydrous DCM (8 mL) was stirred at room temperature for 1 hr until LCMS indicated all starting amine was disappeared and desired product was detected. Then the solution was concentrated to dryness to 7-3 (207 mg, 0.265 mmol, 96.17%) as yellow oil, used as such in the next step.

### Step 3

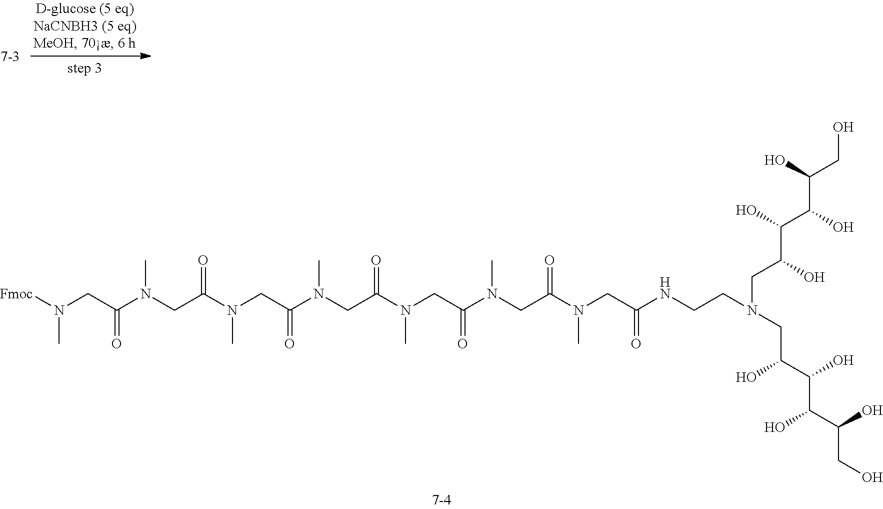

A solution of 7-3 (207 mg, 0.265 mmol) and D-Glucose (239 mg, 1.325 mmol) in anhydrous Methanol (50 mL) was heated at 50° C. for 30 min, then NaCNBH₃ (83 mg, 1.325 mmol) was added. The resulting solution was stirred for another 6 hr at 70° C. until indicated all starting amine was disappeared and the mass of desired product was detected. Then the reaction solution was concentrated and purified by reverse phase liquid chromatography to give 7-4 (147 mg, 0.133 mmol, 50.19%) as a white solid.

### Step 4

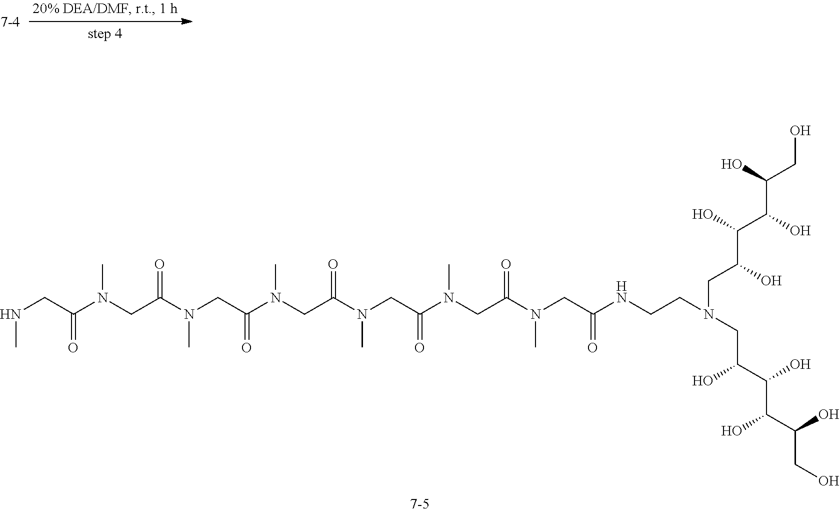 [SMILES: CNCC(=O)N(C)CC(=O)N(C)CC(=O)N(C)CC(=O)N(C)CC(=O)N(C)CC(=O)N(C)CC(=O)NCCN(C[C@@H](O)[C@H](O)[C@@H](O)[C@@H](O)CO)C[C@@H](O)[C@H](O)[C@@H](O)[C@@H](O)CO]

A solution of 7-4 (147 mg, 0.133 mmol) and DEA (1 mL) in anhydrous DMF (4 mL) was stirred at room temperature for 1 hr until LCMS indicated all starting amine was disappeared and desired product was detected. Then the solution was concentrated to dryness to 7-5 (103 mg, 0.116 mmol, 87.41%) as colorless oil, used as such in the next step.

### Step 5

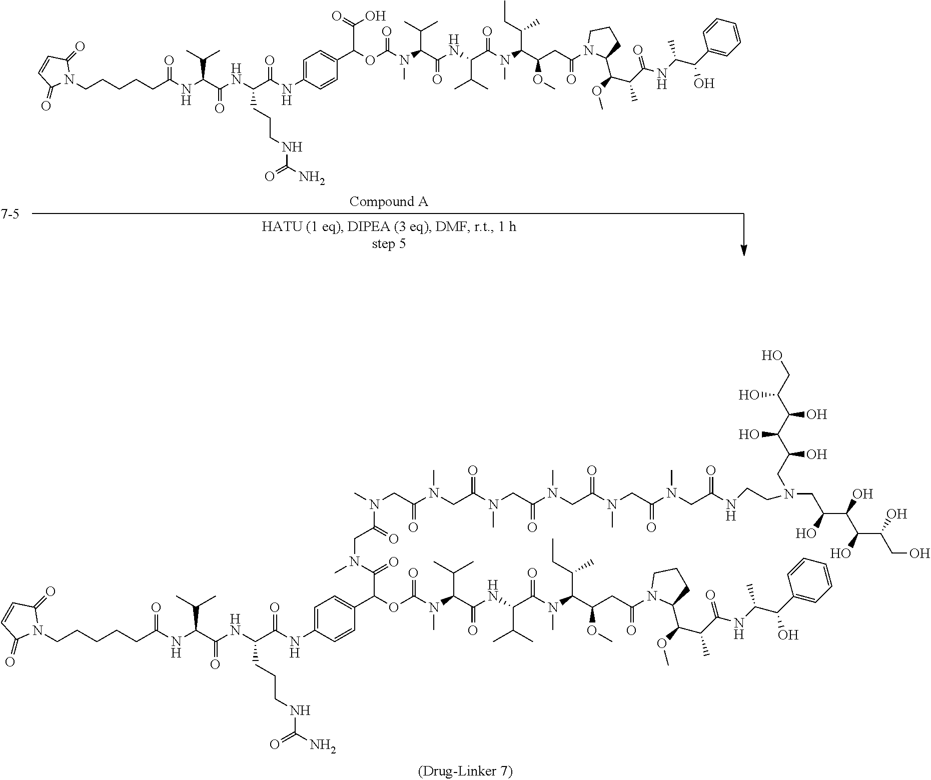

A solution of 7-5 (14 mg, 0.015 mmol), Compound A (20 mg, 0.015 mmol) and HATU (6 mg, 0.015 mmol) in anhydrous DMF (4 mL) was stirred at room temperature for 5 min, then DIPEA (6 mg, 0.045 mmol) was added. The resulting solution was stirred for another 1 hr at r.t. until LCMS indicated complete reaction. Then the reaction solution was purified by prep. HPLC to give drug-linker 7 (15 mg, 0.007 mmol, 44.87%) as a white solid. LCMS, m/z=1115.24 (M/2+H)⁺.

### Example 8: Preparation of Drug-Linker 8

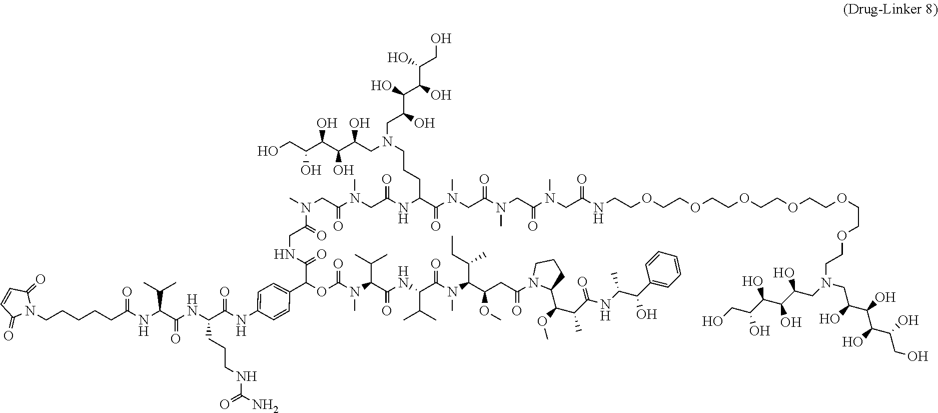 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

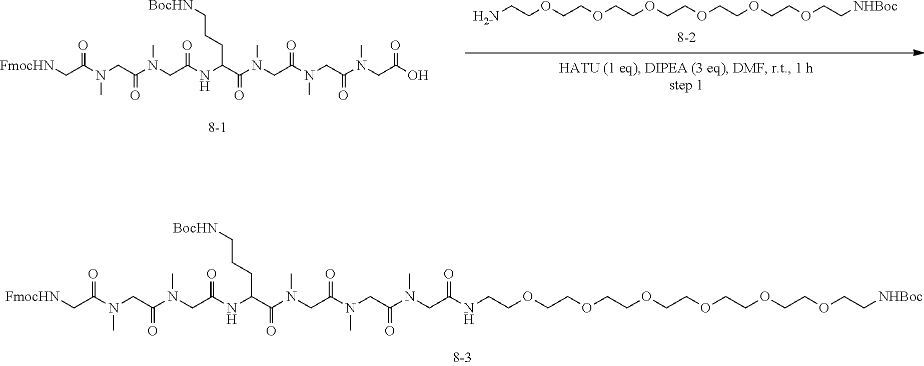

A solution of 8-1 (300 mg, 0.346 mmol), 8-2 (147 mg, 0.346 mmol) and HATU (132 mg, 0.346 mmol) in anhydrous DMF (5 mL) was stirred at room temperature for 5 min, then DIPEA (134 mg, 1.037 mmol) was added. The resulting solution was stirred for another 1 hr at r.t. until LCMS indicated complete reaction. The reaction solution was purified directly by reverse phase liquid chromatography to give 8-3 (310 mg, 0.243 mmol, 70.23%) as a white solid. Purity, 95%.

### Step 2

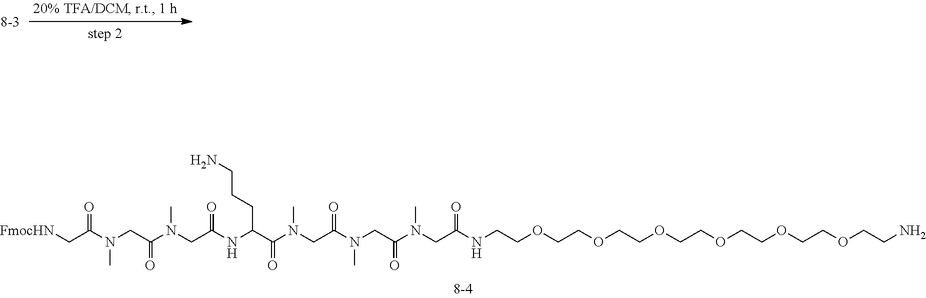

A solution of 8-3 (310 mg, 0.243 mmol) and TFA (1 mL) in anhydrous DCM (4 mL) was stirred at room temperature for 1 hr until LCMS indicated all starting amine was disappeared and desired product was detected. Then the solution was concentrated to dryness to 8-4 (255 mg, 0.238 mmol, 97.78%) as yellow oil, used as such in the next step. Purity, 95%.

### Step 3

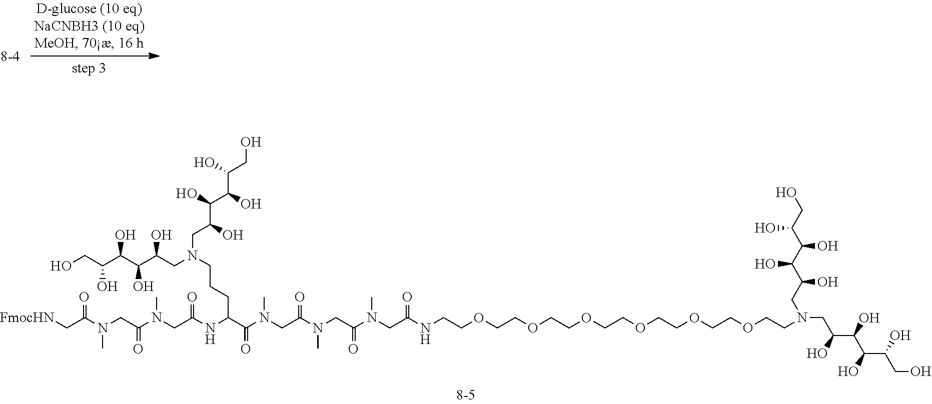

A solution of 8-4 (255 mg, 0.238 mmol) and D-Glucose (428 mg, 2.376 mmol) in anhydrous Methanol (50 mL) was heated at 50° C. for 30 min, then NaCNBH₃ (150 mg, 2.387 mmol) was added. The resulting solution was stirred for another 16 hr at 70° C. until indicated all starting amine was disappeared and the mass of desired product was detected. Then the reaction solution was concentrated and purified by reverse phase liquid chromatography to give 8-5 (185 mg, 0.107 mmol, 44.96%) as a white solid. Purity, 95%.

### Step 4

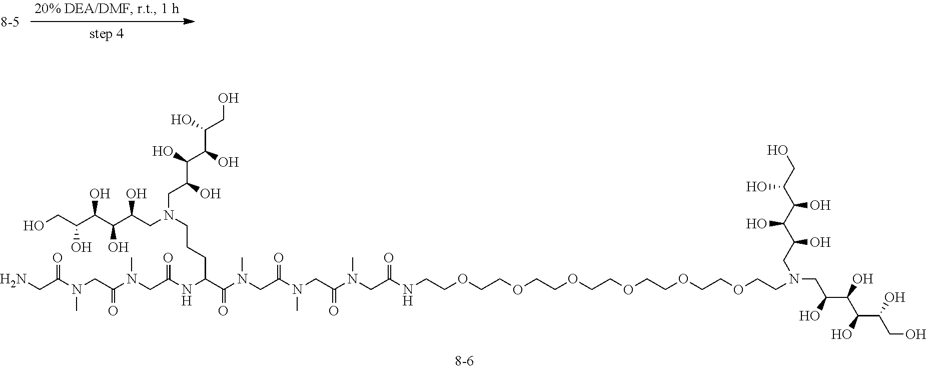 [SMILES: CN(CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 8-5 (185 mg, 0.107 mmol) and DEA (1 mL) in anhydrous DMF (4 mL) was stirred at room temperature for 1 hr until LCMS indicated all starting amine was disappeared and desired product was detected. Then the solution was concentrated to dryness to 8-6 (155 mg, 0.103 mmol, 96.08%) as colorless oil, used as such in the next step. Purity, 95%.

### Step 5

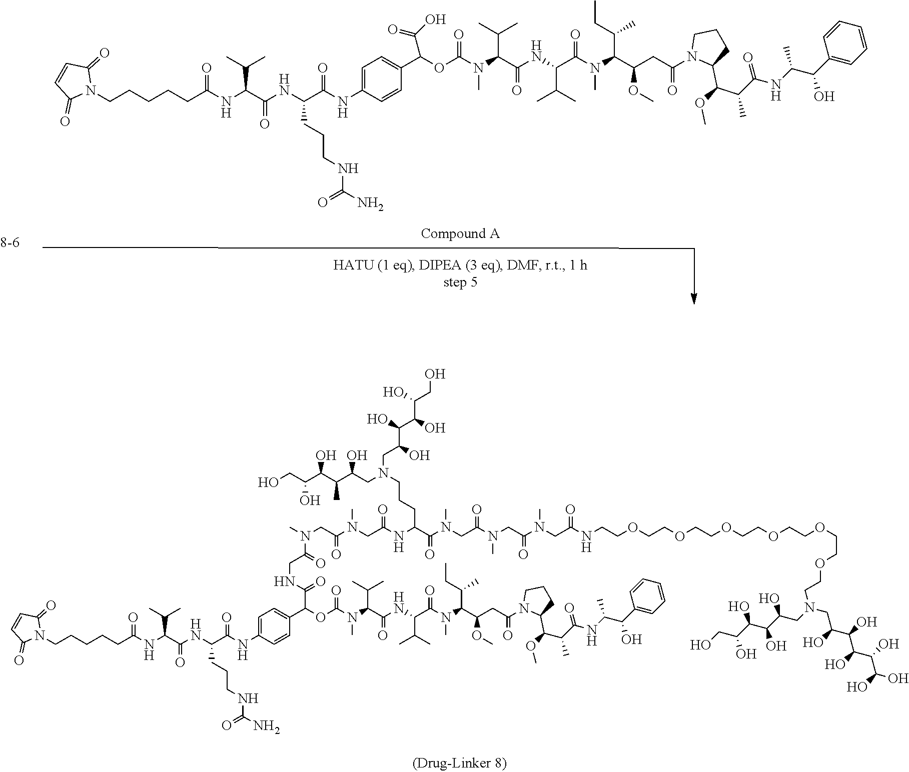

A solution of 8-6 (100 mg, 0.066 mmol), Compound A (90 mg, 0.066 mmol) and HATU (25 mg, 0.066 mmol) in anhydrous DMF (4 mL) was stirred at room temperature for 5 min, then DIPEA (26 mg, 0.201 mmol) was added. The resulting solution was stirred for another 1 hr at r.t. until LCMS indicated complete reaction. Then the reaction solution was purified by prep. HPLC to give drug-linker 8 (10 mg, 0.004 mmol, 5.32%) as a white solid. LCMS, m/z=1425.93 (M/2+H)⁺.

### Example 9: Preparation of Drug-Linker 9

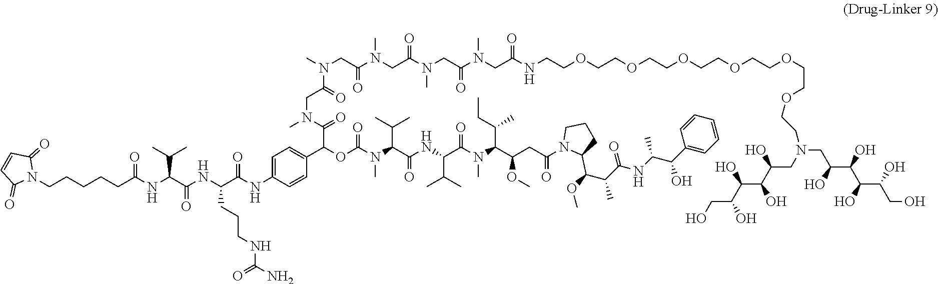 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

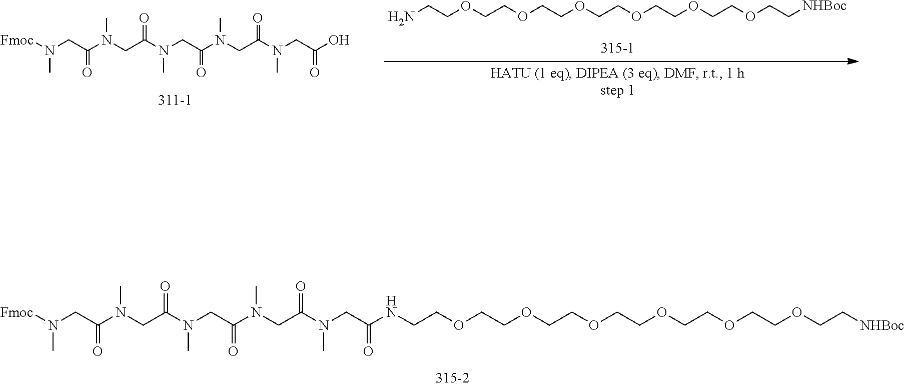

A solution of 311-1 (300 mg, 0.504 mmol), 315-1 (213 mg, 0.504 mmol) and HATU (192 mg, 0.407 mmol) in anhydrous DMF (10 mL) was stirred at room temperature for 5 min, then DIPEA (196 mg, 1.517 mmol) was added. The resulting solution was stirred for another 1 hr at r.t. until LCMS indicated complete reaction. The reaction solution was purified directly by reverse phase liquid chromatography to give 315-2 (315 mg, 0.314 mmol, 62.30%) as a white solid.

### Step 2

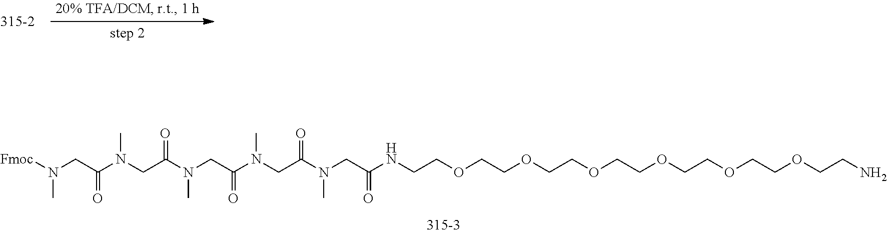

A solution of 315-2 (315 mg, 0.314 mmol) and TFA (2 mL) in anhydrous DCM (8 mL) was stirred at room temperature for 1 hr until LCMS indicated all starting amine was disappeared and desired product was detected. Then the solution was concentrated to dryness to 315-3 (264 mg, 0.293 mmol, 93.31%) as yellow oil, used as such in the next step.

### Step 3

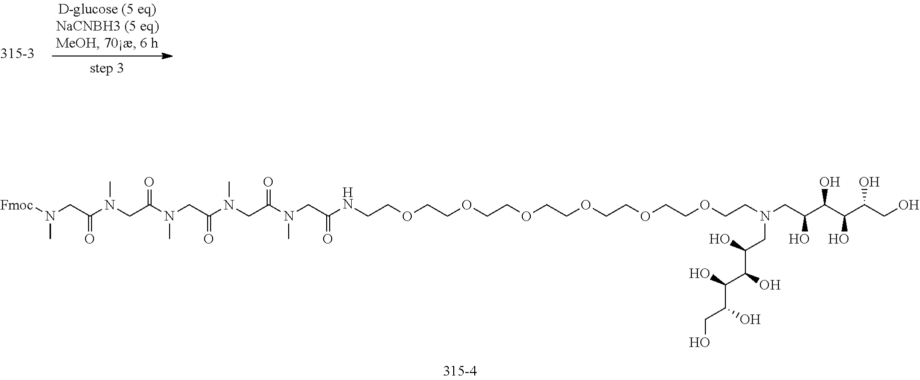

A solution of 315-3 (264 mg, 0.293 mmol) and D-Glucose (263 mg, 1.461 mmol) in anhydrous Methanol (50 mL) was heated at 50° C. for 30 min, then NaCNBH₃ (92 mg, 1.464 mmol) was added. The resulting solution was stirred for another 6 hr at 70° C. until indicated all starting amine was disappeared and the mass of desired product was detected. Then the reaction solution was concentrated and purified by reverse phase liquid chromatography to give 315-4 (233 mg, 0.189 mmol, 64.51%) as a white solid.

### Step 4

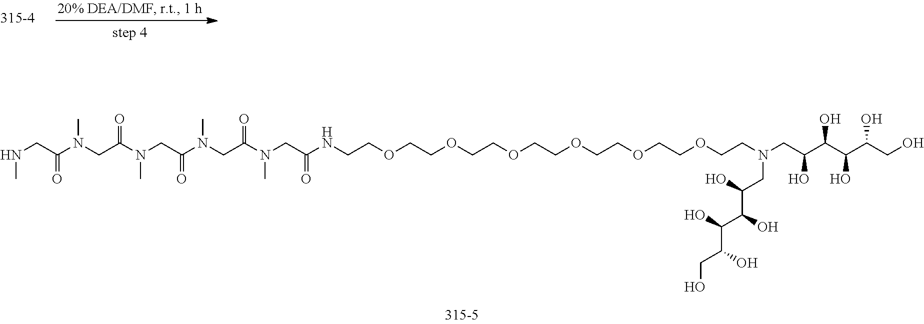 [SMILES: CNCC(=O)N(C)CC(=O)N(C)CC(=O)N(C)CC(=O)N(C)CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-4 (233 mg, 0.189 mmol) and DEA (1 mL) in anhydrous DMF (4 mL) was stirred at room temperature for 1 hr until LCMS indicated all starting amine was disappeared and desired product was detected. Then the solution was concentrated to dryness to 315-5 (177 mg, 0.176 mmol, 93.12%) as colorless oil, used as such in the next step.

### Step 5

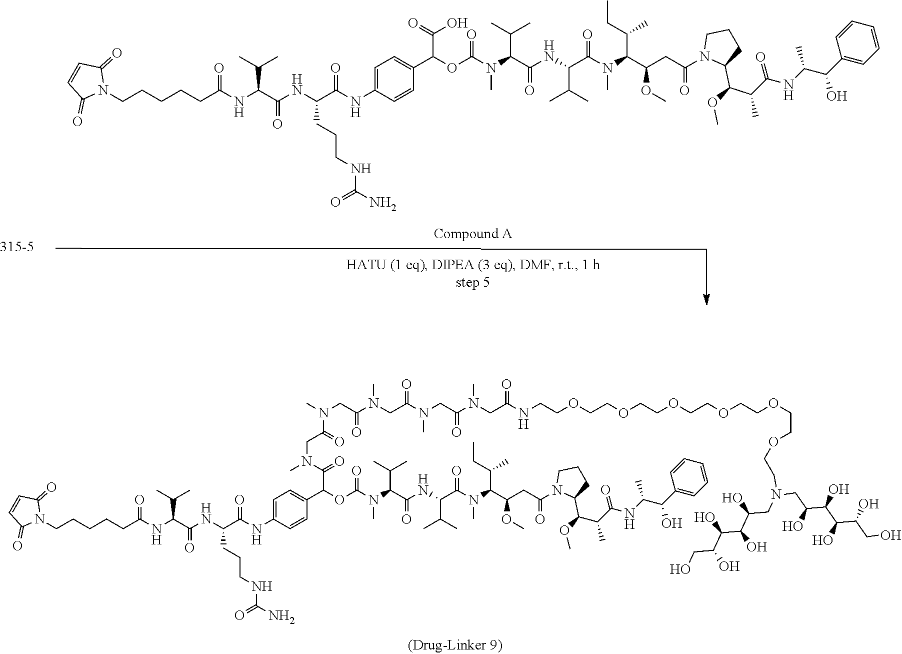

A solution of 315-5 (30 mg, 0.030 mmol), Compound A (40 mg, 0.030 mmol) and HATU (11 mg, 0.030 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 8 (18 mg, 0.007 mmol, 25.53%) as a white solid. LCMS, m/z=1176.02 (M/2+H)⁺.

### Example 10: Preparation of Drug-Linker 10

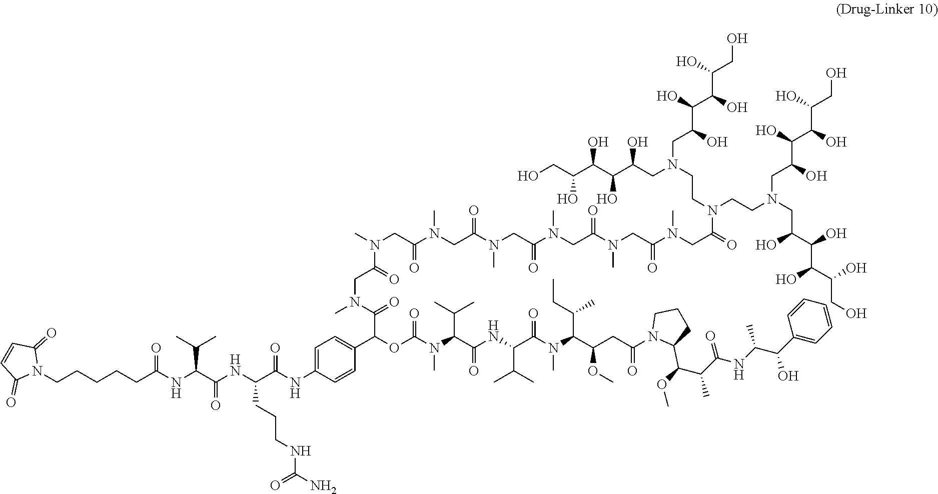 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

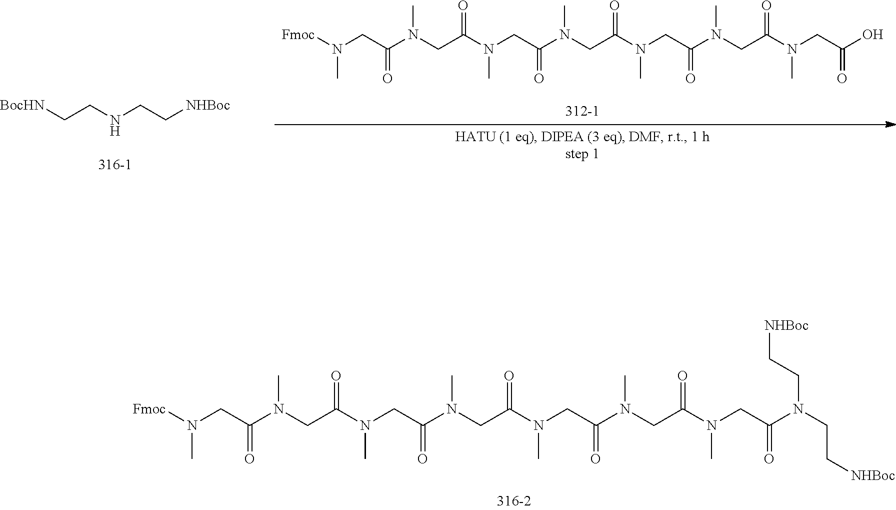

A solution of 316-1 (82 mg, 0.270 mmol), 312-1 (200 mg, 0.270 mmol) and HATU (103 mg, 0.270 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 316-2 (205 mg, 0.200 mmol, 74.07%) as a white solid.

### Step 2

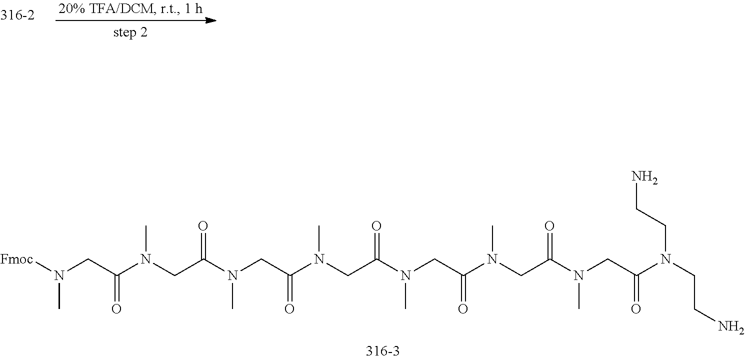

A solution of 316-2 (205 mg, 0.200 mmol) and TFA (1 mL) in anhydrous DCM (4 mL) was stirred at room temperature for 1 hr until LCMS indicated all starting amine was disappeared and desired product was detected. Then the solution was concentrated to dryness to 316-3 (157 mg, 0.191 mmol, 93.17%) as yellow oil, used as such in the next step.

### Step 3

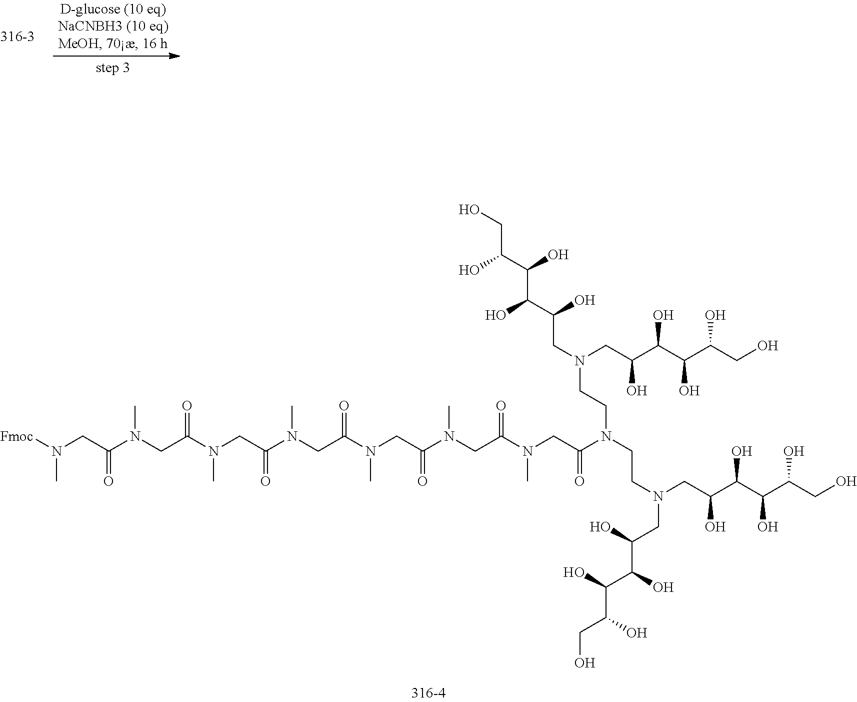

A solution of 316-3 (157 mg, 0.191 mmol) and D-Glucose (172 mg, 0.955 mmol) in anhydrous Methanol (50 mL) was heated at 50° C. for 30 min, then NaCNBH₃ (60 mg, 0.955 mmol) was added. The resulting solution was stirred for another 16 hr at 70° C. until indicated all starting amine was disappeared and the mass of desired product was detected. Then the reaction solution was concentrated and purified by reverse phase liquid chromatography to give 316-4 (135 mg, 0.091 mmol, 47.77%) as a white solid.

### Step 4

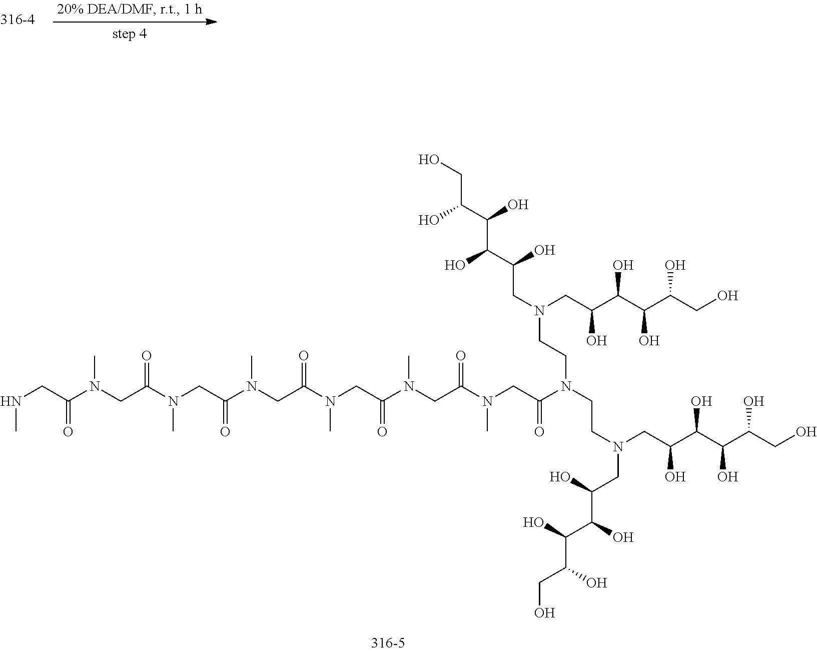 [SMILES: CNCC(=O)N(C)CC(=O)N(C)CC(=O)N(C)CC(=O)N(C)CC(=O)N(C)CC(=O)N(C)CC(=O)N(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO]

A solution of 316-4 (135 mg, 0.091 mmol) and DEA (1 mL) in anhydrous DMF (4 mL) was stirred at room temperature for 1 hr until LCMS indicated all starting amine was disappeared and desired product was detected. Then the solution was concentrated to dryness to 316-5 (114 mg, 0.090 mmol, 98.90%) as colorless oil, used as such in the next step.

### Step 5

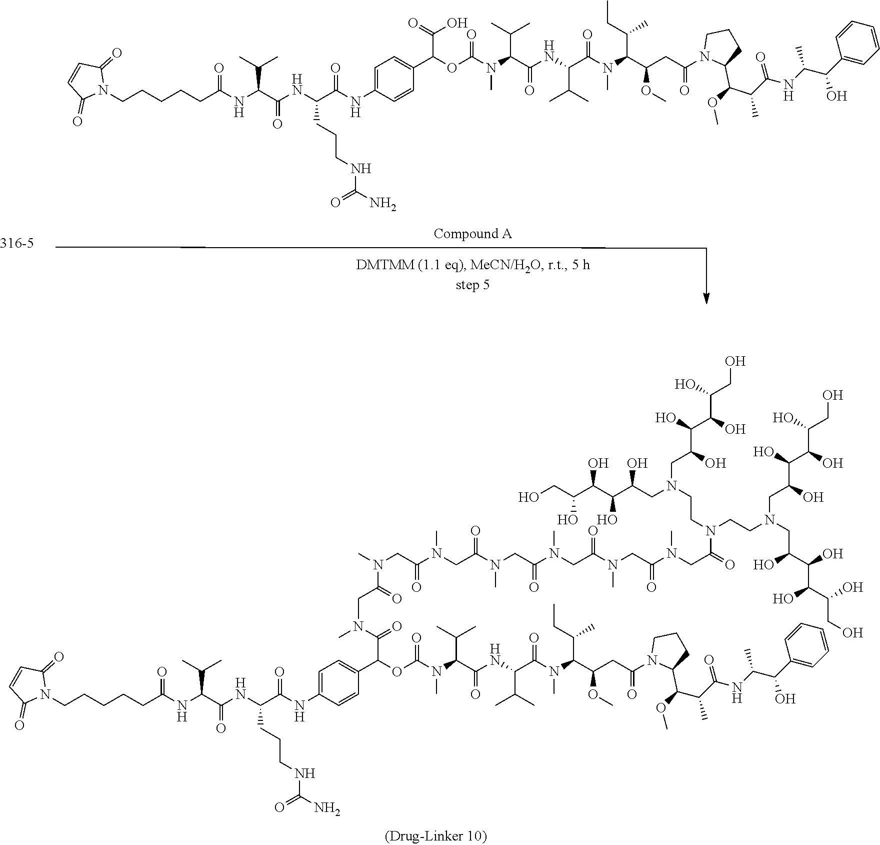

A solution of 316-5 (100 mg, 0.080 mmol), Compound A (108 mg, 0.080 mmol) and DMTMM (24 mg, 0.088 mmol) in H₂O (4 mL) and MeCN (4 mL) was stirred at room temperature for 5 hr. Then the reaction solution was purified by prep. HPLC to give drug-linker 10 (7 mg, 0.003 mmol, 3.37%) as a white solid. LCMS, m/z=1300.87 (M/2+H)⁺.

### Example 11: Preparation of Drug-Linker 11

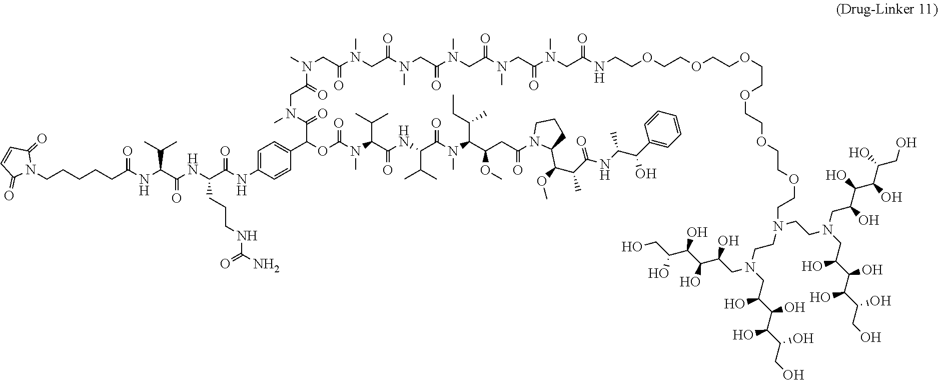 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)N(C)CC(=O)N(C)CC(=O)N(C)CC(=O)N(C)CC(=O)N(C)CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

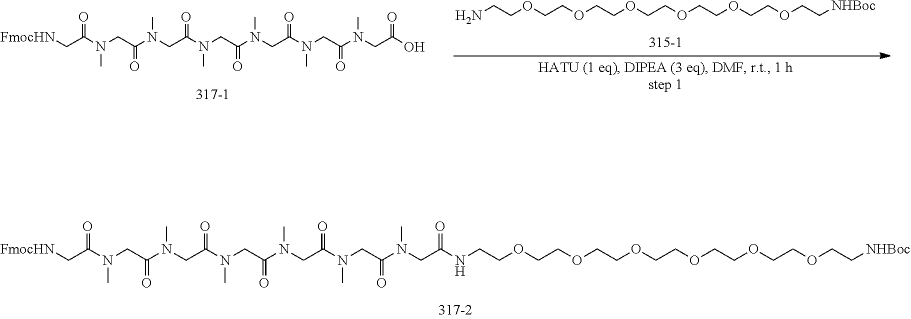

A solution of 317-1 (400 mg, 0.553 mmol), 315-1 (235 mg, 0.554 mmol) and HATU (210 mg, 0.553 mmol) in anhydrous DMF (6 mL) was stirred at room temperature for 5 min, then DIPEA (214 mg, 1.656 mmol) was added. The resulting solution was stirred for another 1 hr at room temperature until LCMS indicated complete reaction.

The reaction solution was purified directly by reverse phase liquid chromatography to give 317-2 (410 mg, 0.363 mmol, 65.59%) as a white solid.

### Step 2

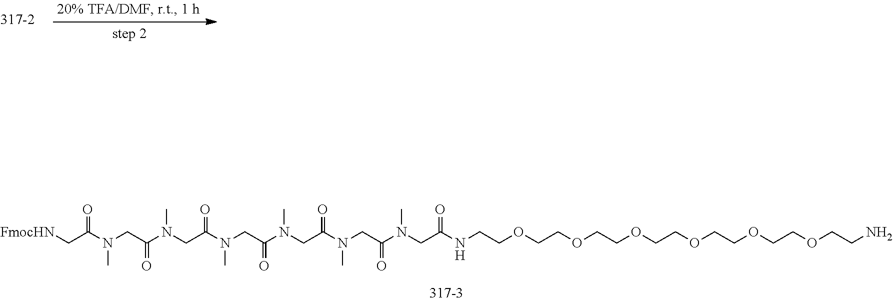

A solution of 317-2 (410 mg, 0.363 mmol) and TFA (2 mL) in anhydrous DCM (8 mL) was stirred at room temperature for 1 hr until LCMS indicated all starting amine was disappeared and desired product was detected. Then the solution was concentrated to dryness to 317-3 (365 mg, 0.354 mmol, 97.60%) as yellow oil, used as such in the next step.

### Step 3

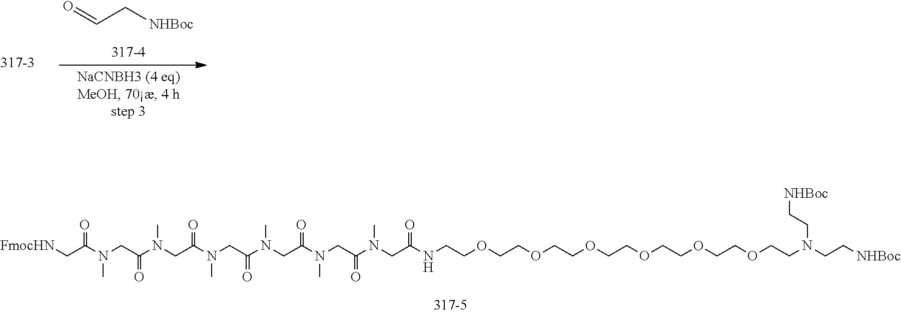

A solution of 317-3 (365 mg, 0.354 mmol) and 317-4 (338 mg, 2.123 mmol) in anhydrous Methanol (50 mL) was heated at 50° C. for 30 min, then NaCNBH₃ (134 mg, 2.132 mmol) was added. The resulting solution was stirred for another 4 hr at 70° C. until indicated all starting amine was disappeared and the mass of desired product was detected. Then the reaction solution was concentrated and purified by reverse phase liquid chromatography to give 317-5 (310 mg, 0.235 mmol, 66.51%) as a white solid.

### Step 4

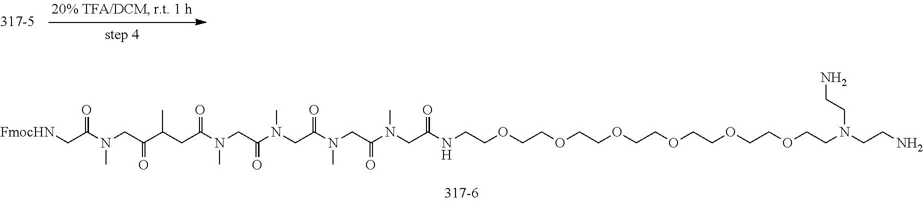

A solution of 317-5 (310 mg, 0.235 mmol) and TFA (2 mL) in anhydrous DCM (8 mL) was stirred at room temperature for 1 hr until LCMS indicated all starting amine was disappeared and desired product was detected. Then the solution was concentrated to dryness to 317-6 (250 mg, 0.224 mmol, 95.30%) as yellow oil, used as such in the next step.

### Step 5

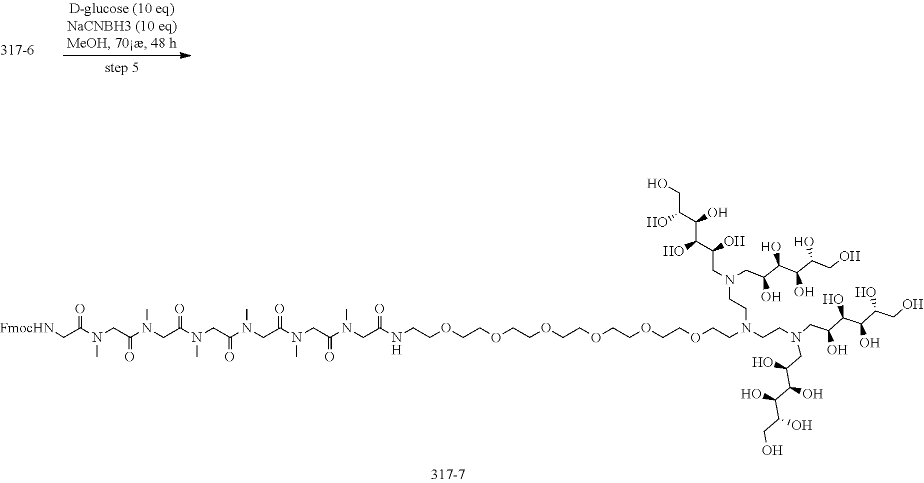

A solution of 317-6 (250 mg, 0.224 mmol) and D-Glucose (484 mg, 2.687 mmol) in anhydrous Methanol (50 mL) was heated at 50° C. for 30 min, then NaCNBH₃ (169 mg, 2.689 mmol) was added. The resulting solution was stirred for another 48 hr at 70° C. until indicated all starting amine was disappeared and the mass of desired product was detected. Then the reaction solution was concentrated and purified by reverse phase liquid chromatography to give 317-7 (174 mg, 0.098 mmol, 43.81%) as a white solid.

### Step 6

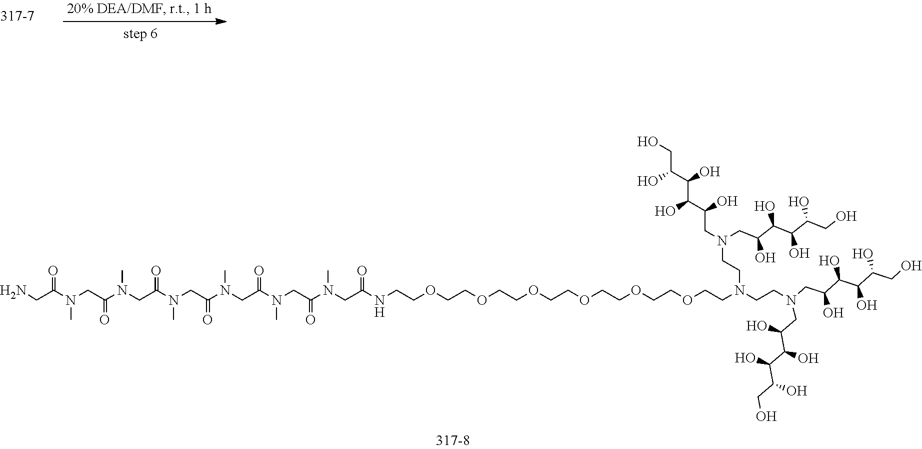 [SMILES: CN(CC(=O)NCCOCCOCCOCCOCCOCCOCCN(CCN(C[C@H](O)[C@@H](O)[C@H](O)[C@H](O)CO)C[C@H](O)[C@@H](O)[C@H](O)[C@H](O)CO)CCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)CN(C)C(=O)CN(C)C(=O)CN(C)C(=O)CN]

A solution of 317-7 (174 mg, 0.098 mmol) and DEA (1 mL) in anhydrous DMF (4 mL) was stirred at room temperature for 1 hr until LCMS indicated all starting amine was disappeared and desired product was detected. Then the solution was concentrated to dryness to 317-8 (145 mg, 0.094 mmol, 95.41%) as colorless oil, used as such in the next step.

### Step 7

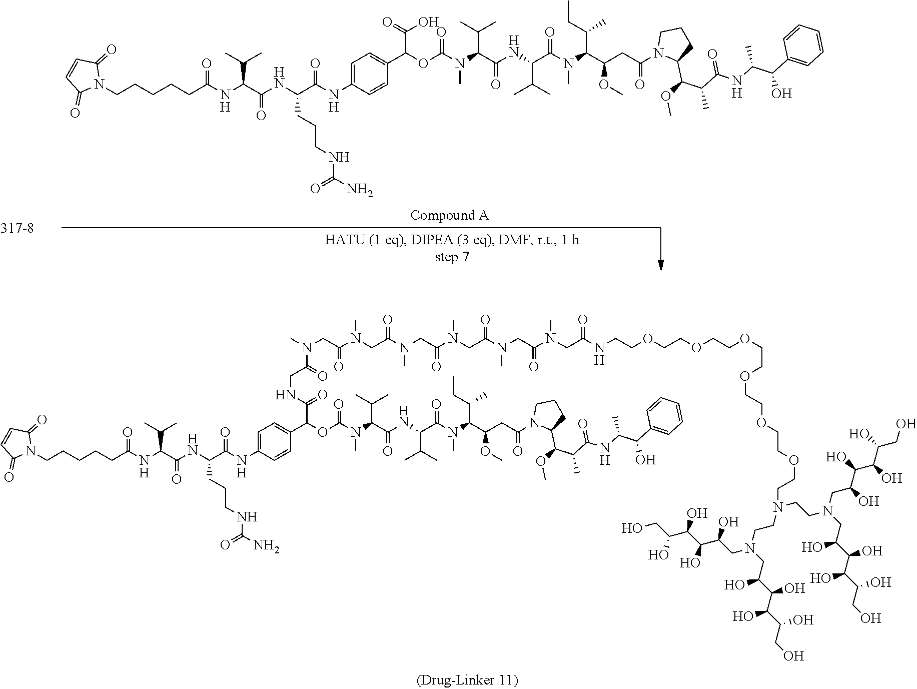

A solution of 317-8 (80 mg, 0.052 mmol), Compound A (70 mg, 0.052 mmol) and HATU (20 mg, 0.052 mmol) in anhydrous DMF (4 mL) was stirred at room temperature for 5 min, then DIPEA (20 mg, 0.155 mmol) was added. The resulting solution was stirred for another 1 hr at r.t. until LCMS indicated complete reaction. Then the reaction solution was purified by prep. HPLC to give drug-linker 11 (40 mg, 0.014 mmol, 26.59%) as a white solid. LCMS, m/z=1447.20 (M/2+H)⁺.

### Example 12: Preparation of Drug-Linker 12

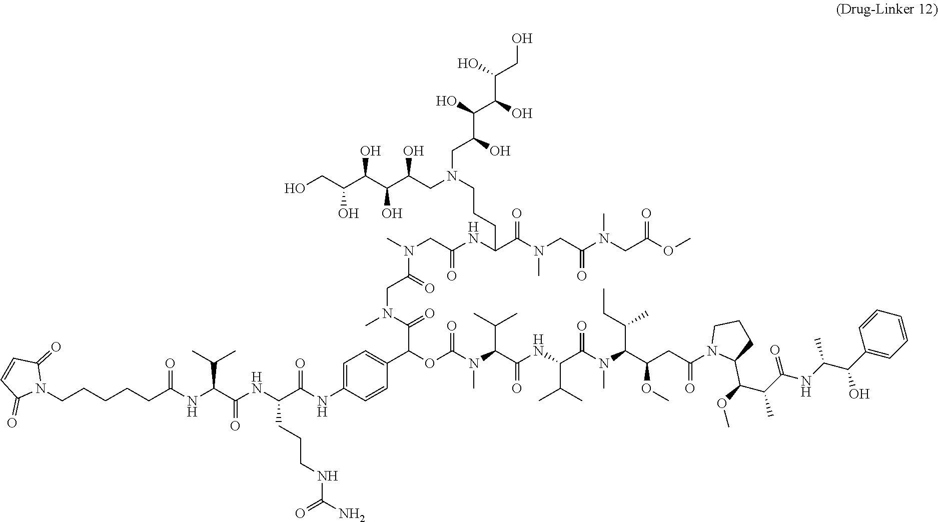 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

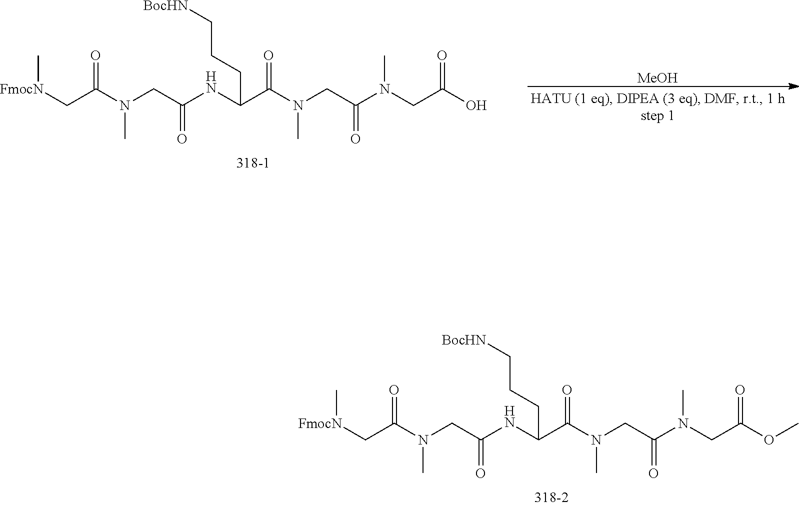

A solution of 318-1 (200 mg, 0.271 mmol), MeOH (43 mg, 1.342 mmol) and HATU (103 mg, 0.271 mmol) in anhydrous DMF (10 mL) was stirred at room temperature for 5 min, then DIPEA (105 mg, 0.812 mmol) was added. The resulting solution was stirred for another 1 hr at r.t. until LCMS indicated complete reaction. The reaction solution was purified directly by reverse phase liquid chromatography to give 318-2 (167 mg, 0.222 mmol, 81.92%) as a white solid.

### Step 2

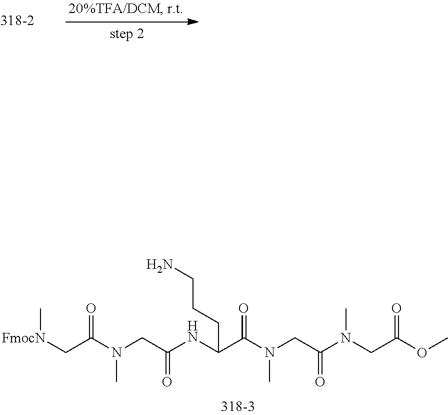

A solution of 318-2 (167 mg, 0.222 mmol) and TFA (1 mL) in anhydrous DCM (4 mL) was stirred at room temperature for 1 hr until LCMS indicated all starting amine was disappeared and desired product was detected. Then the solution was concentrated to dryness to 318-3 (135 mg, 0.207 mmol, 93.24%) as yellow oil, used as such in the next step.

### Step 3

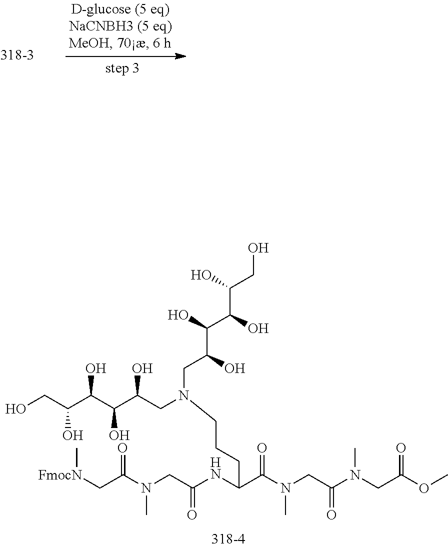

A solution of 318-3 (135 mg, 0.207 mmol) and D-Glucose (186 mg, 1.032 mmol) in anhydrous Methanol (50 mL) was heated at 50° C. for 30 min, then NaCNBH₃ (65 mg, 1.034 mmol) was added. The resulting solution was stirred for another 6 hr at 70° C. until indicated all starting amine was disappeared and the mass of desired product was detected. Then the reaction solution was concentrated and purified by reverse phase liquid chromatography to give 318-4 (121 mg, 0.123 mmol, 59.42%) as a white solid.

### Step 4

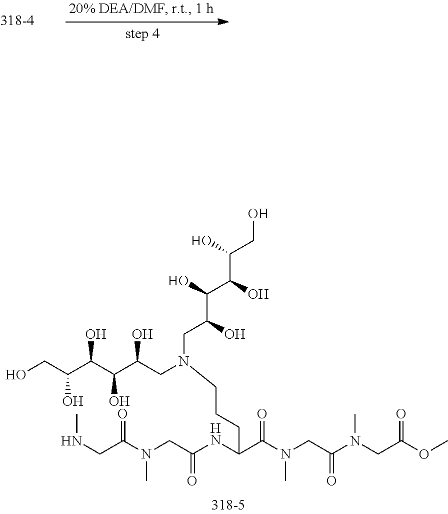 [SMILES: CNCC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)OC]

A solution of 318-4 (121 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 318-5 (75 mg, 0.099 mmol, 80.49%) as colorless oil, used as such in the next step.

### Step 5

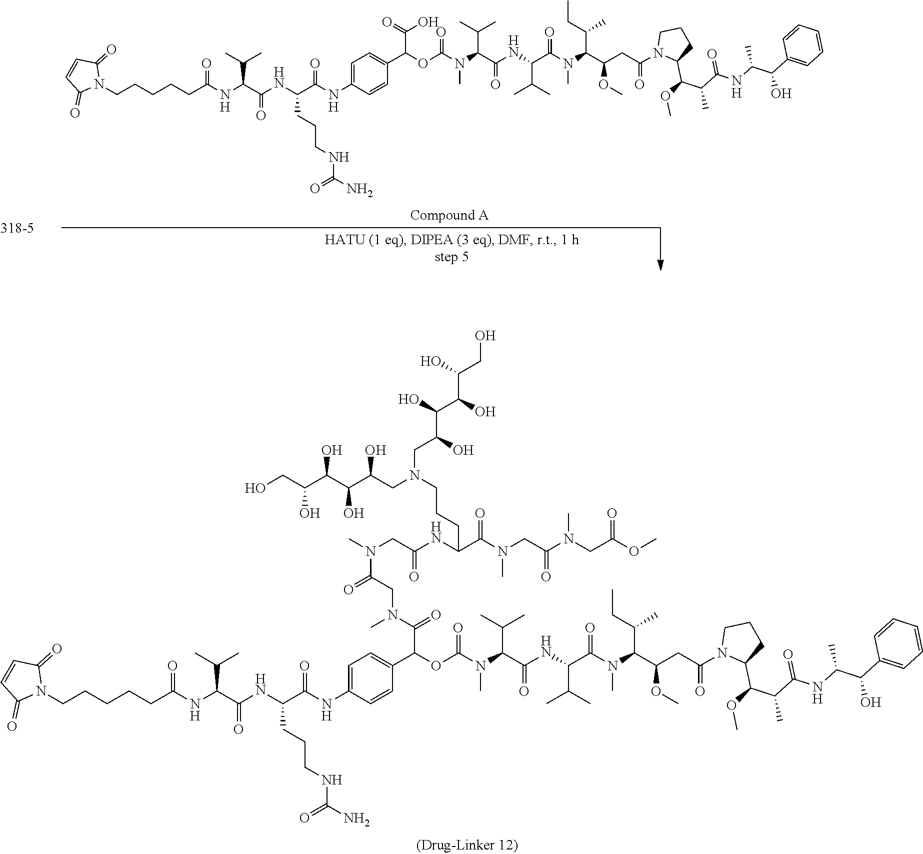

A solution of 318-5 (17 mg, 0.022 mmol), Compound A (30 mg, 0.022 mmol) and HATU (8 mg, 0.022 mmol) in anhydrous DMF (4 mL) was stirred at room temperature for 5 min, then DIPEA (8 mg, 0.062 mmol) was added. The resulting solution was stirred for another 1 hr at r.t. until LCMS indicated complete reaction. Then the reaction solution was purified by prep. HPLC to give drug-linker 12 (16 mg, 0.008 mmol, 36.36%) as a white solid. LCMS, m/z=1051.28 (M/2+H)⁺.

### Example 13: Preparation of Drug-Linker 13

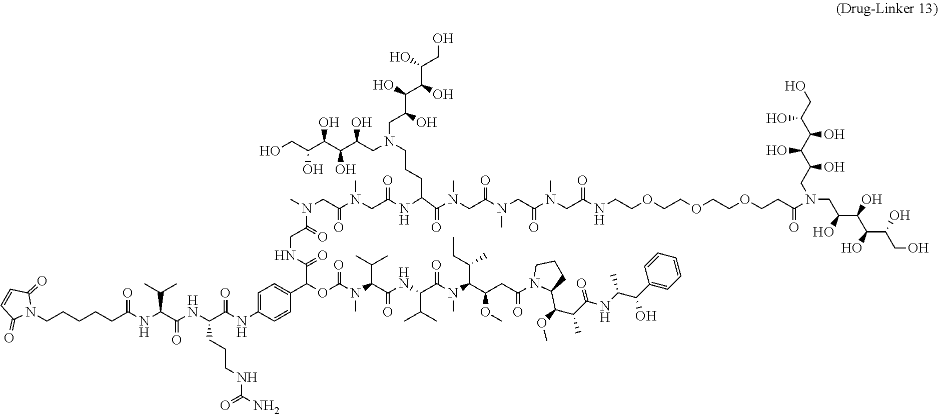 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

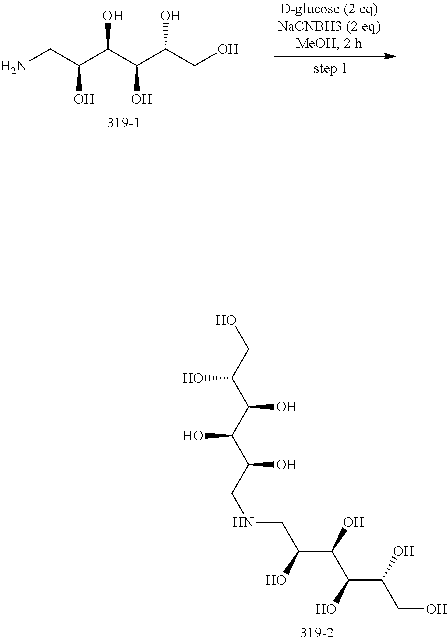 [SMILES: NC[C@H](O)[C@@H](O)[C@H](O)[C@H](O)CO ; OC[C@@H](O)[C@@H](O)[C@H](O)[C@@H](O)CNC[C@H](O)[C@@H](O)[C@H](O)[C@H](O)CO]

A solution of 319-1 (2.0 g, 0.011 mol) and D-Glucose (4.0 g, 0.022 mol) in anhydrous Methanol (50 mL) was heated at 50° C. for 30 min, then NaCNBH₃ (1.4 g, 0.022 mol) was added. The resulting solution was stirred for another 2 hr at 70° C. until indicated all starting amine was disappeared and the mass of desired product was detected. Then the reaction solution was concentrated and purified by reverse phase liquid chromatography to give 319-2 (2.6 g, 7.529 mmol, 68.44%) as a white solid.

### Step 2

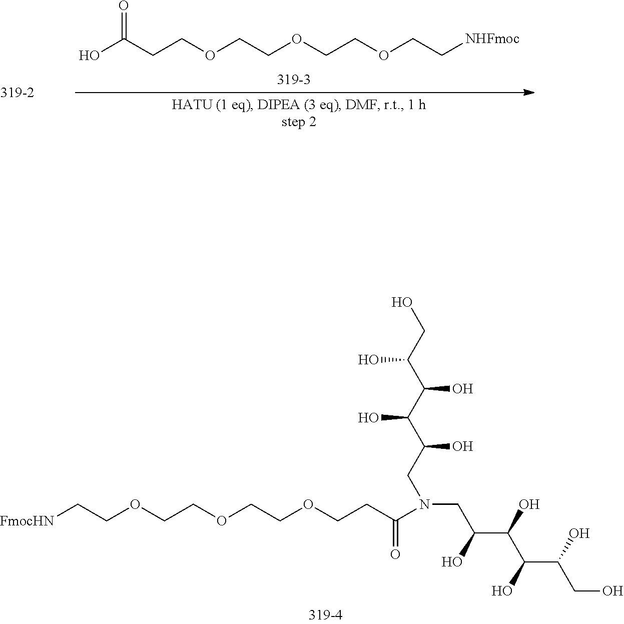

A solution of 319-2 (2.6 g, 7.529 mmol), 319-3 (3.3 g, 7.529 mmol) and HATU (2.9 g, 7.529 mmol) in anhydrous DMF (10 mL) was stirred at room temperature for 5 min, then DIPEA (2.9 g, 22.586 mmol) was added. The resulting solution was stirred for another 1 hr at r.t. until LCMS indicated complete reaction. The reaction solution was purified directly by reverse phase liquid chromatography to give 319-4 (1.5 g, 1.946 mmol, 25.85%) as a white solid.

### Step 3

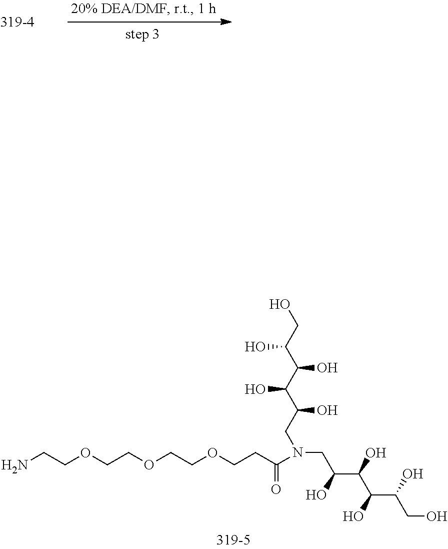 [SMILES: NCCOCCOCCOCCC(=O)N(C[C@H](O)[C@@H](O)[C@H](O)[C@H](O)CO)C[C@H](O)[C@@H](O)[C@H](O)[C@H](O)CO]

A solution of 319-4 (1.5 g, 1.946 mmol) and DEA (2 mL) in anhydrous DMF (8 mL) was stirred at room temperature for 1 hr until LCMS indicated all starting amine was disappeared and desired product was detected. Then the solution was concentrated to dryness to 319-5 (950 mg, 1.732 mmol, 89.00%) as colorless oil, used as such in the next step.

### Step 4

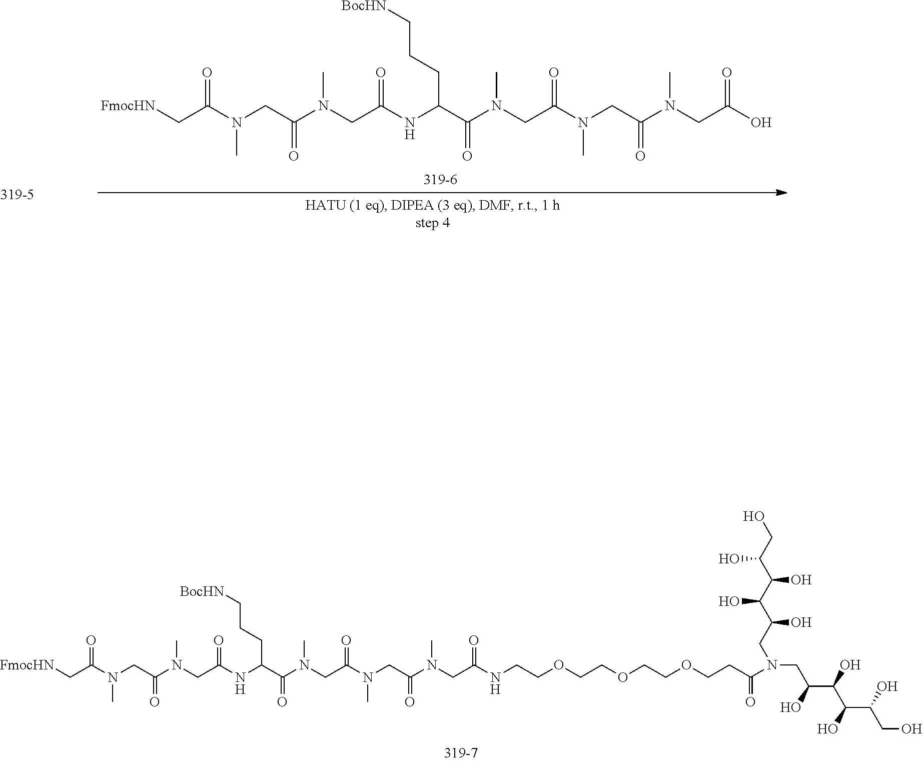

A solution of 319-5 (950 mg, 1.732 mmol), 319-6 (1501 mg, 1.732 mmol) and HATU (658 mg, 1.732 mmol) in anhydrous DMF (10 mL) was stirred at room temperature for 5 min, then DIPEA (670 mg, 5.184 mmol) was added. The resulting solution was stirred for another 1 hr at r.t. until LCMS indicated complete reaction. The reaction solution was purified directly by reverse phase liquid chromatography to give 319-7 (700 mg, 0.501 mmol, 28.93%) as a white solid.

### Step 5

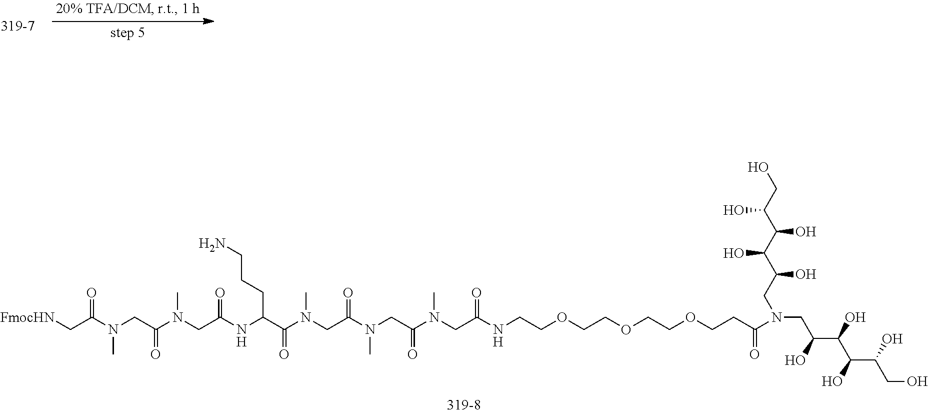

A solution of 319-7 (300 mg, 0.215 mmol) and TFA (1 mL) in anhydrous DCM (4 mL) was stirred at room temperature for 1 hr until LCMS indicated all starting amine was disappeared and desired product was detected. Then the solution was concentrated to dryness to 319-8 (245 mg, 0.189 mmol, 87.91%) as yellow oil, used as such in the next step.

### Step 6

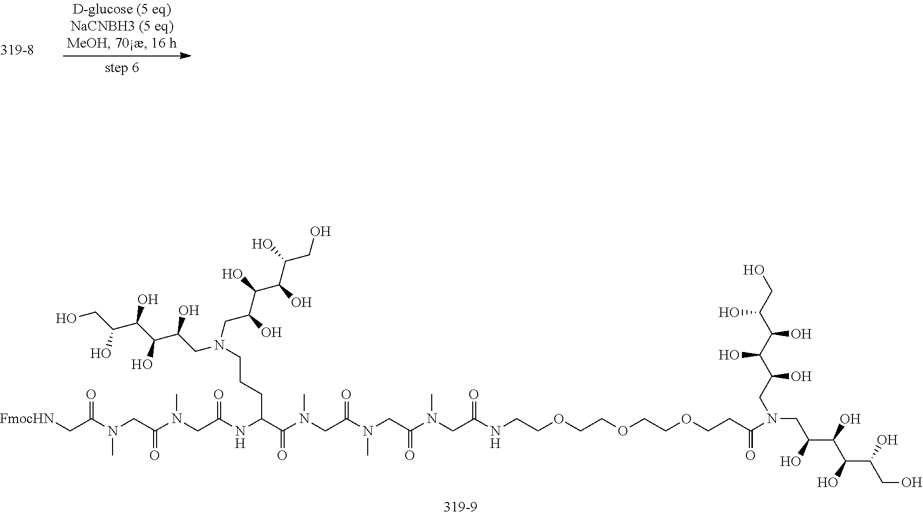

A solution of 319-8 (245 mg, 0.189 mmol) and D-Glucose (170 mg, 0.945 mmol) in anhydrous Methanol (50 mL) was heated at 50° C. for 30 min, then NaCNBH₃ (60 mg, 0.945 mmol) was added. The resulting solution was stirred for another 16 hr at 70° C. until indicated all starting amine was disappeared and the mass of desired product was detected. Then the reaction solution was concentrated and purified by reverse phase liquid chromatography to give 319-9 (140 mg, 0.086 mmol, 45.50%) as a white solid.

### Step 7

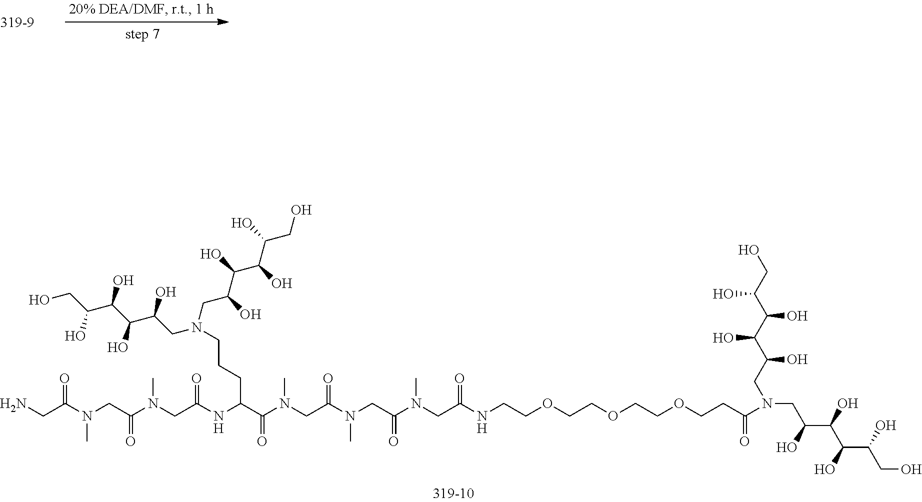 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 319-9 (140 mg, 0.086 mmol) and DEA (1 mL) in anhydrous DMF (4 mL) was stirred at room temperature for 1 hr until LCMS indicated all starting amine was disappeared and desired product was detected. Then the solution was concentrated to dryness to 319-10 (112 mg, 0.080 mmol, 93.02%) as colorless oil, used as such in the next step.

### Step 8

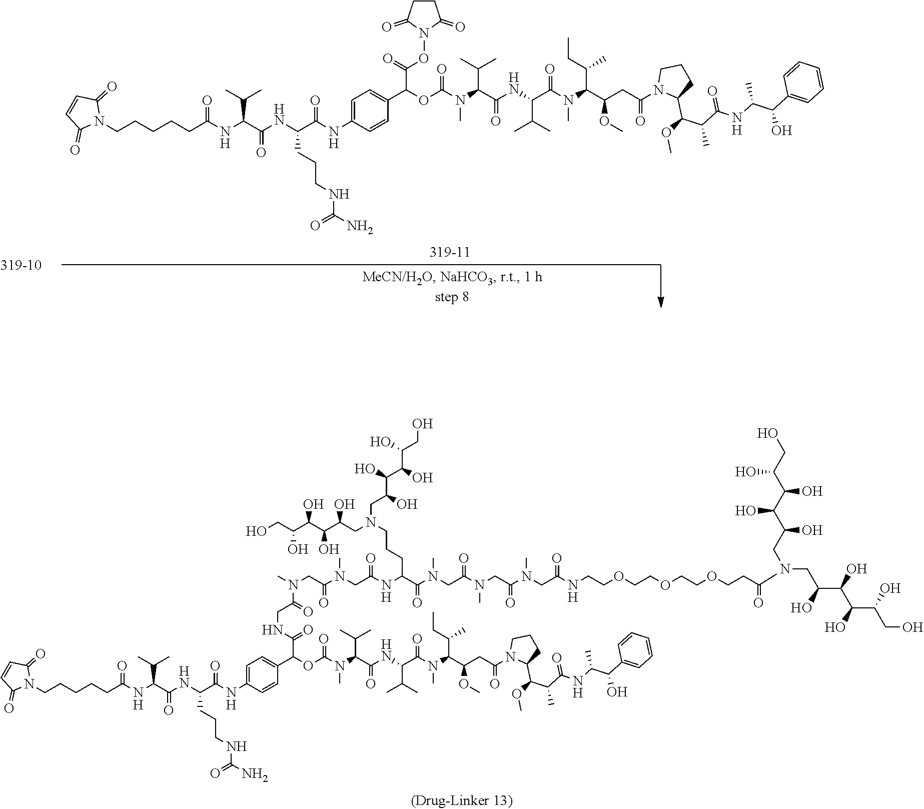

A solution of 319-10 (112 mg, 0.080 mmol) and 319-11 (116 mg, 0.080 mmol) in H₂O (3 mL) and MeCN (3 mL) was adjusted to pH=8 by aqueous sodium bicarbonate. The resulting solution was stirred for another 1 hr at r.t. until LCMS indicated complete reaction. Then the reaction solution was purified by prep. HPLC to give drug-linker 13 (8 mg, 0.003 mmol, 3.75%) as a white solid. LCMS, m/z=916.23 (M/3+H)⁺.

### Example 14: Preparation of Drug-Linker 14

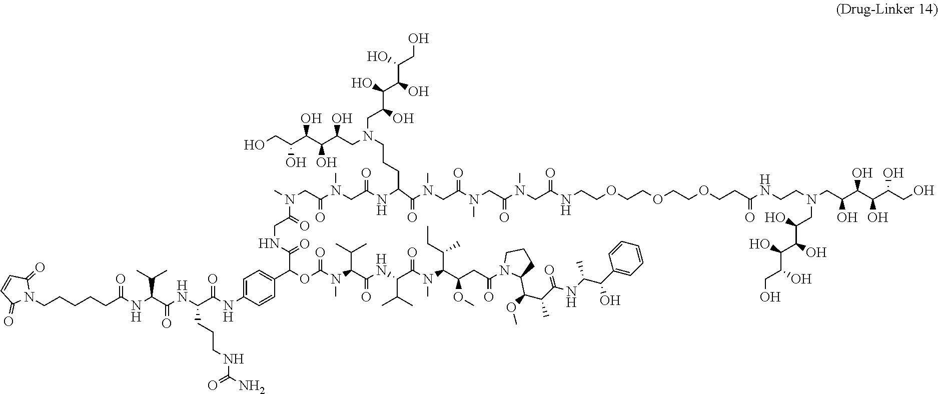 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(=O)N(C)CC(=O)N(C)CC(=O)NC(CCCN(C[C@H](O)[C@@H](O)[C@H](O)[C@H](O)CO)C[C@H](O)[C@@H](O)[C@H](O)[C@H](O)CO)C(=O)N(C)CC(=O)N(C)CC(=O)N(C)CC(=O)NCCOCCOCCOCCC(=O)NCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

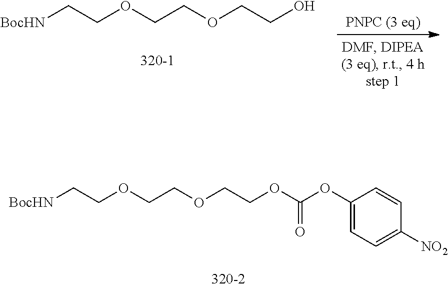 [SMILES: CC(C)(C)OC(=O)NCCOCCOCCO ; CC(C)(C)OC(=O)NCCOCCOCCOC(=O)Oc1ccc([N+](=O)[O-])cc1]

A solution of 320-1 (1.0 g, 4.011 mol) and DIPEA (1.6 g, 12.380 mol) in anhydrous DMF (15 mL) was stirred at room temperature for 5 min, then PNPC (3.7 g, 12.163 mmol) was added. The resulting solution was stirred for another 4 hr at r.t. until LCMS indicated all starting amine was disappeared and desired product was detected. Then the reaction solution was concentrated and purified by reverse phase liquid chromatography to give 320-2 (943 mg, 2.276 mmol, 56.73%) as a yellow solid.

### Step 2

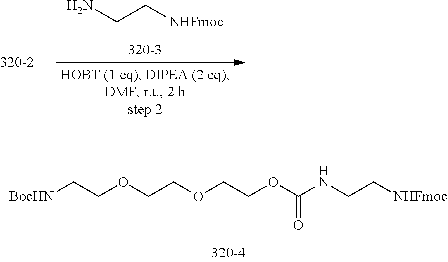

A solution of 320-2 (943 mg, 2.276 mmol), 320-3 (642 mg, 2.274 mmol) and HOBT (307 mg, 2.274 mmol) in anhydrous DMF (50 mL) was stirred at room temperature, then DIPEA (588 mg, 4.550 mmol) was added. The resulting solution was stirred for another 1 hr at r.t. until LCMS indicated all starting amine was disappeared and desired product was detected. The reaction solution was purified directly by reverse phase liquid chromatography to give 320-4 (850 mg, 1.524 mmol, 66.97%) as a white solid.

### Step 3

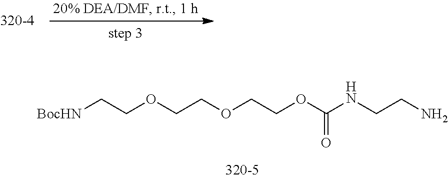 [SMILES: CC(C)(C)OC(=O)NCCOCCOCCOC(=O)NCCN]

A solution of 320-4 (850 mg, 1.524 mmol) and DEA (2 mL) in anhydrous DMF (8 mL) was stirred at room temperature for 1 hr until LCMS indicated all starting amine was disappeared and desired product was detected. Then the solution was concentrated to dryness to 320-5 (491 mg, 1.464 mmol, 96.06%) as colorless oil, used as such in the next step.

### Step 4

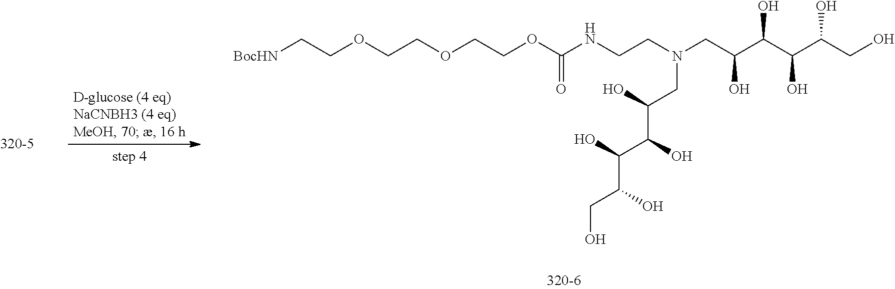 [SMILES: CC(C)(C)OC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-5 (491 mg, 1.464 mmol) and D-Glucose (1.05 g, 5.828 mmol) in anhydrous Methanol (50 mL) was heated at 50° C. for 30 min, then NaCNBH₃ (368 mg, 5.856 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-6 (470 mg, 0.708 mmol, 48.37%) as a white solid.

### Step 5

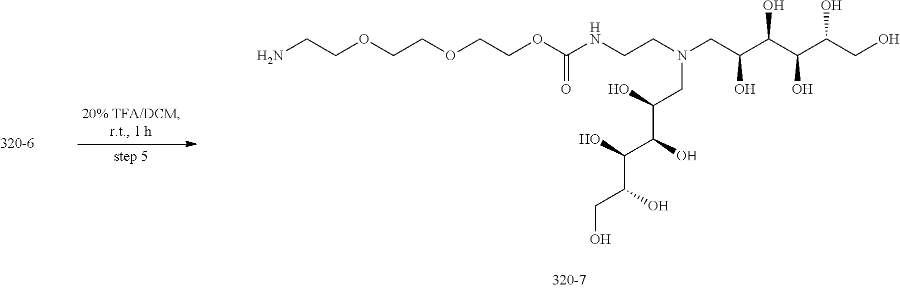 [SMILES: NCCOCCOCCOC(=O)NCCN(C[C@H](O)[C@@H](O)[C@H](O)[C@H](O)CO)C[C@H](O)[C@@H](O)[C@H](O)[C@H](O)CO]

A solution of 320-6 (470 mg, 0.708 mmol) and TFA (2 mL) in anhydrous DCM (8 mL) was stirred at room temperature for 1 hr until LCMS indicated all starting amine was disappeared and desired product was detected. Then the solution was concentrated to dryness to 320-7 (385 mg, 0.683 mmol, 96.48%) as yellow oil, used as such in the next step.

### Step 6

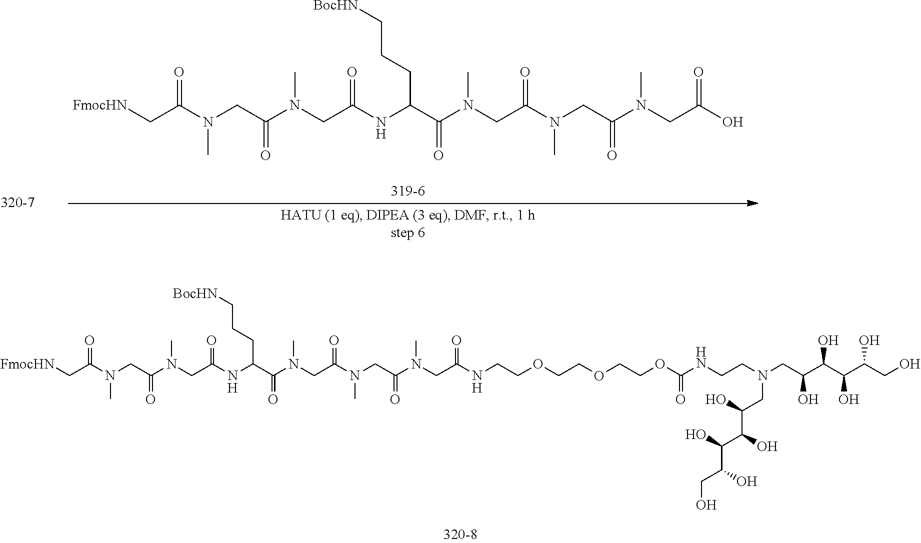

A solution of 320-7 (385 mg, 0.683 mmol), 319-6 (592 mg, 0.683 mmol) and HATU (260 mg, 0.684 mmol) in anhydrous DMF (6 mL) was stirred at room temperature for 5 min, then DIPEA (265 mg, 2.050 mmol) was added. The resulting solution was stirred for another 1 hr at r.t. until LCMS indicated complete reaction. The reaction solution was purified directly by reverse phase liquid chromatography to give 320-8 (413 mg, 0.292 mmol, 42.81%) as a white solid.

### Step 7

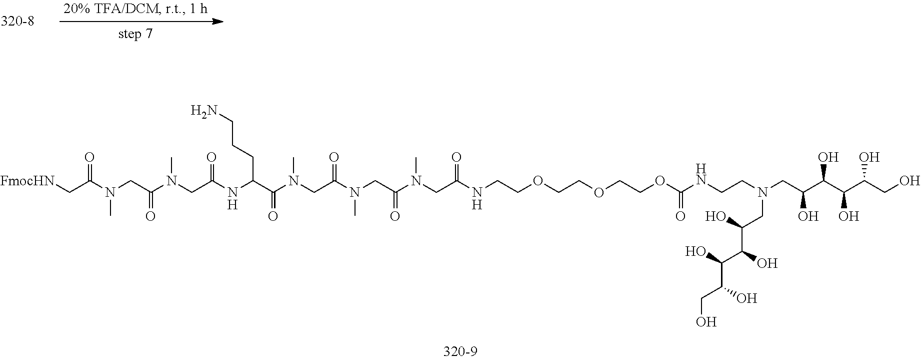

A solution of 320-8 (413 mg, 0.292 mmol) and TFA (2 mL) in anhydrous DCM (8 mL) was stirred at room temperature for 1 hr until LCMS indicated all starting amine was disappeared and desired product was detected. Then the solution was concentrated to dryness to 320-9 (370 mg, 0.262 mmol, 89.70%) as yellow oil, used as such in the next step.

### Step 8

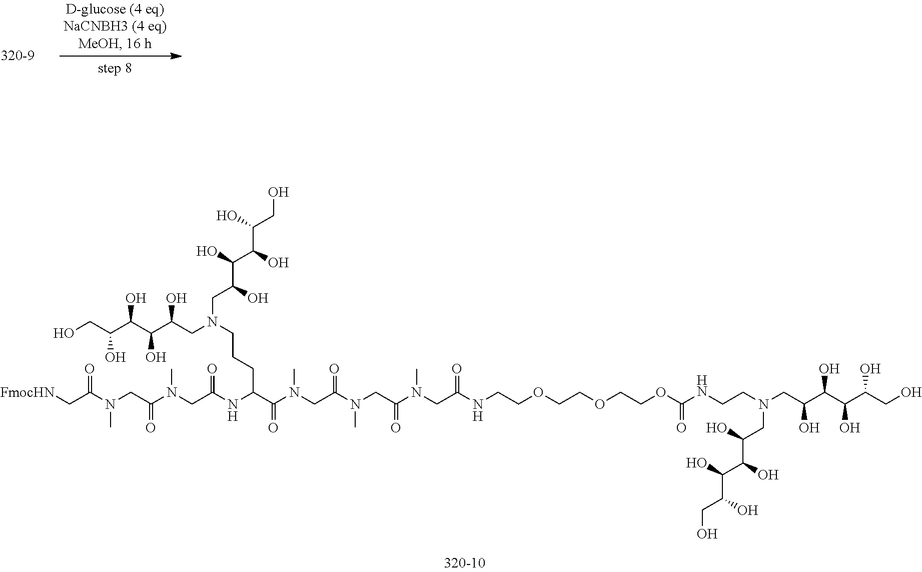

A solution of 320-9 (370 mg, 0.262 mmol) and D-Glucose (203 mg, 1.127 mmol) in anhydrous Methanol (40 mL) was heated at 50° C. for 30 min, then NaCNBH₃ (71 mg, 1.130 mmol) was added. The resulting solution was stirred for another 16 hr at 70° C. until indicated all starting amine was disappeared and the mass of desired product was detected. Then the reaction solution was concentrated and purified by reverse phase liquid chromatography to give 320-10 (210 mg, 0.128 mmol, 48.85%) as a white solid.

### Step 9

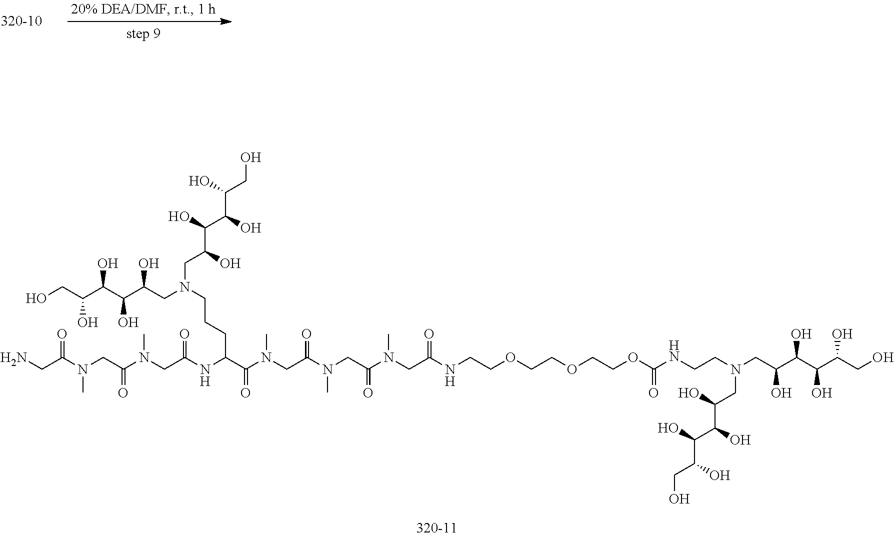 [SMILES: CN(CC(=O)NCCOCCOCCOC(=O)NCCN(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CN]

A solution of 320-10 (210 mg, 0.128 mmol) and DEA (1 mL) in anhydrous DMF (4 mL) was stirred at room temperature for 1 hr until LCMS indicated all starting amine was disappeared and desired product was detected. Then the solution was concentrated to dryness to 320-11 (175 mg, 0.123 mmol, 96.38%) as colorless oil, used as such in the next step.

### Step 10

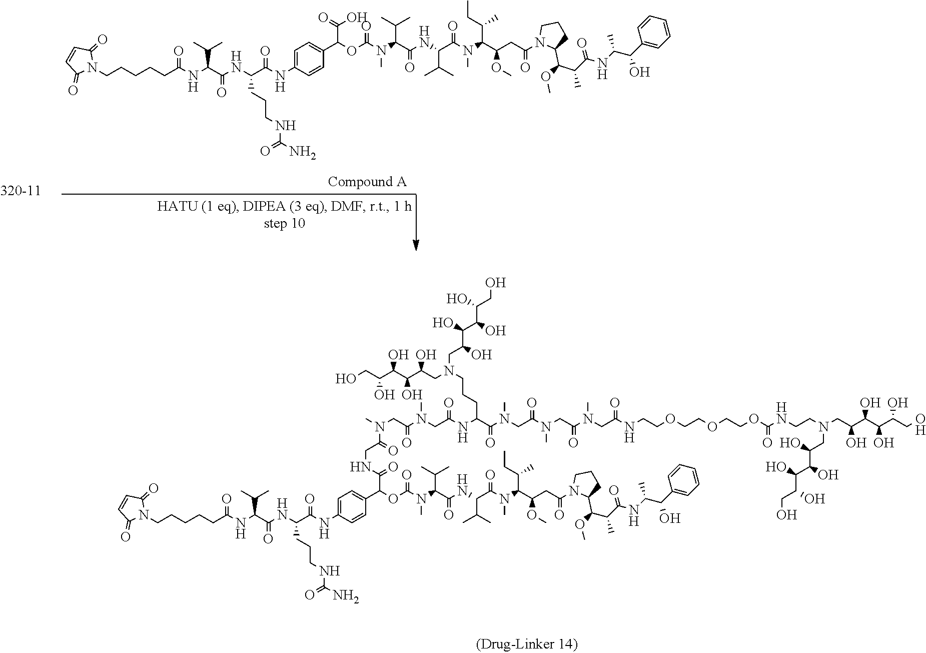

A solution of 320-11 (42 mg, 0.030 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 14 (16 mg, 0.006 mmol, 19.32%) as a white solid. LCMS, m/z=1381.18 (M/2+H)⁺.

### Example 15: Preparation of Drug-Linker 15

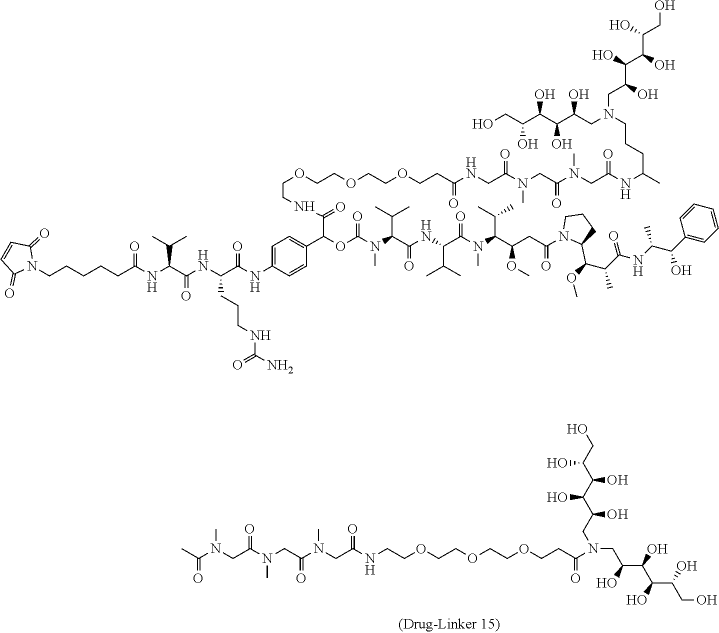

### Step 1

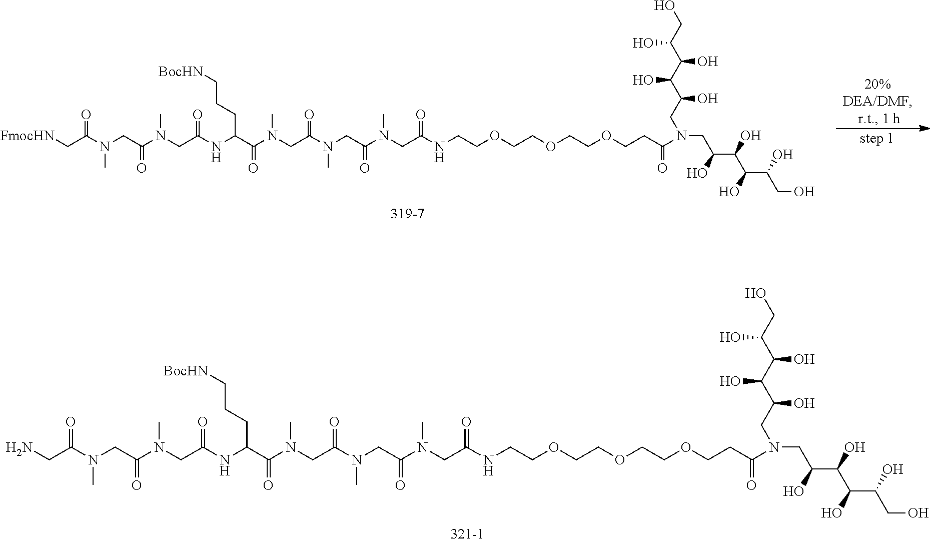

A solution of 319-7 (350 mg, 0.250 mmol) and DEA (1 mL) in anhydrous DMF (4 mL) was stirred at room temperature for 1 hour until LCMS indicated all starting amine was disappeared and desired product was detected. Then the solution was concentrated to dryness to 321-1 (280 mg, 0.238 mmol, 95.29%) as colorless oil, used as such in the next step.

### Step 2

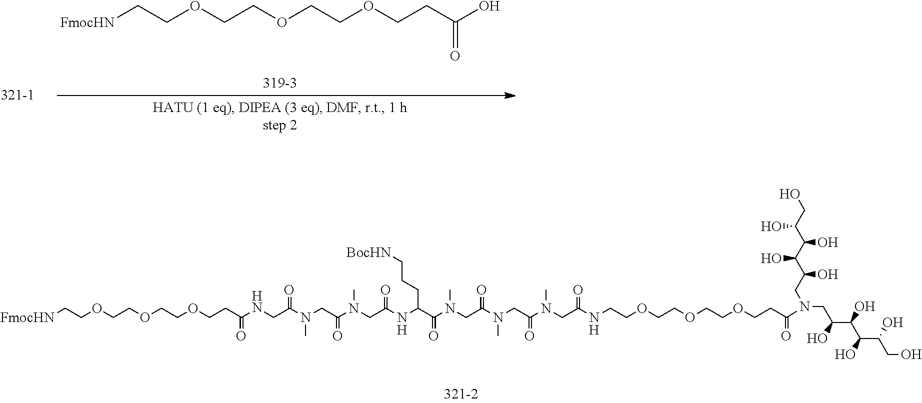

A solution of 321-1 (280 mg, 0.238 mmol), 319-3 (105 mg, 0.237 mmol) and HATU (90 mg, 0.237 mmol) in anhydrous DMF (10 mL) was stirred at room temperature for 5 min, then DIPEA (92 mg, 0.712 mmol) was added. The resulting solution was stirred for another 1 hour at r.t. until LCMS indicated complete reaction. The reaction solution was purified directly by reverse phase liquid chromatography to give 321-2 (265 mg, 0.166 mmol, 69.56%) as a white solid.

### Step 3

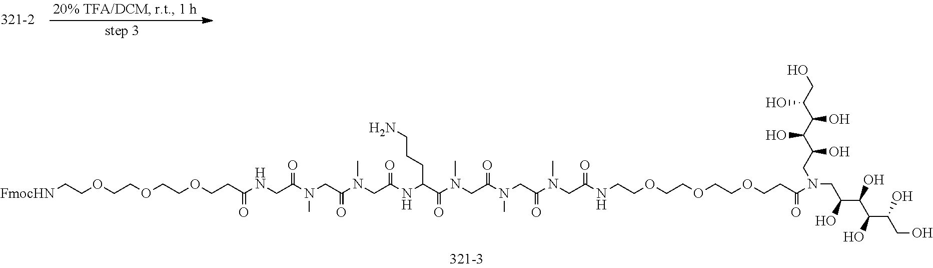

A solution of 321-2 (265 mg, 0.166 mmol) and TFA (1 mL) in anhydrous DCM (4 mL) was stirred at room temperature for 1 hr until LCMS indicated all starting amine was disappeared and desired product was detected. Then the solution was concentrated to dryness to 321-3 (243 mg, 0.162 mmol, 97.55%) as yellow oil, used as such in the next step.

### Step 4

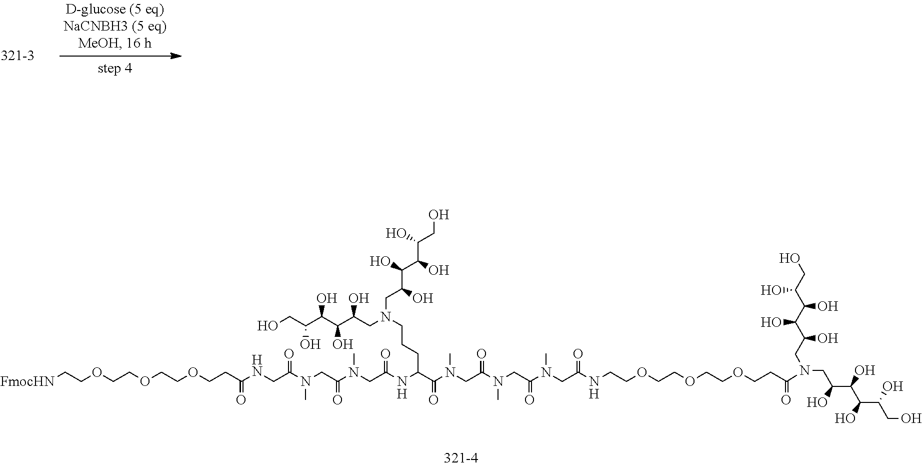

A solution of 321-3 (243 mg, 0.162 mmol) and D-Glucose (146 mg, 0.810 mmol) in anhydrous Methanol (50 mL) was heated at 50° C. for 30 min, then NaCNBH₃ (51 mg, 0.812 mmol) was added. The resulting solution was stirred for another 16 hr at 70° C. until indicated all starting amine was disappeared and the mass of desired product was detected. Then the reaction solution was concentrated and purified by reverse phase liquid chromatography to give 321-4 (155 mg, 0.085 mmol, 52.31%) as a white solid.

### Step 5

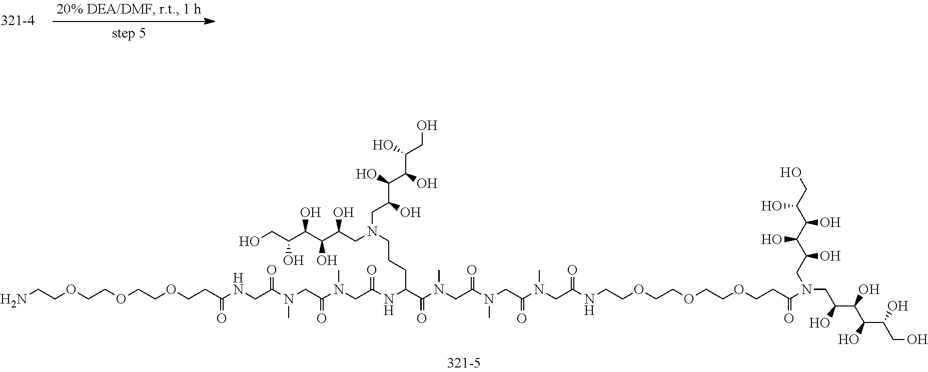 [SMILES: CN(CC(=O)NCCOCCOCCOCCC(=O)N(C[C@H](O)[C@@H](O)[C@H](O)[C@H](O)CO)C[C@H](O)[C@@H](O)[C@H](O)[C@H](O)CO)C(=O)CN(C)C(=O)CN(C)C(=O)C(CCCN(C[C@H](O)[C@@H](O)[C@H](O)[C@H](O)CO)C[C@H](O)[C@@H](O)[C@H](O)[C@H](O)CO)NC(=O)CN(C)C(=O)CN(C)C(=O)CNC(=O)CCOCCOCCOCCN]

A solution of 321-4 (155 mg, 0.085 mmol) and DEA (1 mL) in anhydrous DMF (4 mL) was stirred at room temperature for 1 hr until LCMS indicated all starting amine was disappeared and desired product was detected. Then the solution was concentrated to dryness to 321-5 (130 mg, 0.081 mmol, 95.19%) as colorless oil, used as such in the next step.

### Step 6

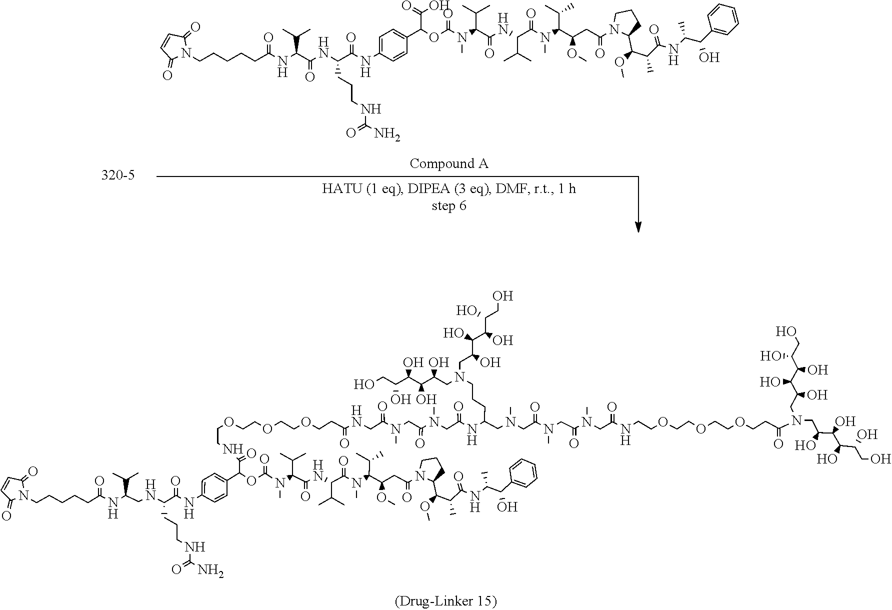

A solution of 321-5 (130 mg, 0.081 mmol), Compound A (110 mg, 0.081 mmol) and HATU (31 mg, 0.081 mmol) in anhydrous DMF (4 mL) was stirred at room temperature for 5 min, then DIPEA (31 mg, 0.240 mmol) was added. The resulting solution was stirred for another 1 hr at r.t. until LCMS indicated complete reaction. Then the reaction solution was purified by Prep. HPLC to give drug-linker 15 (24 mg, 0.008 mmol, 9.88%) as a white solid. LCMS, m/z=984.06 (M/3+H)⁺.

### Example 16: Preparation of Drug-Linker 16

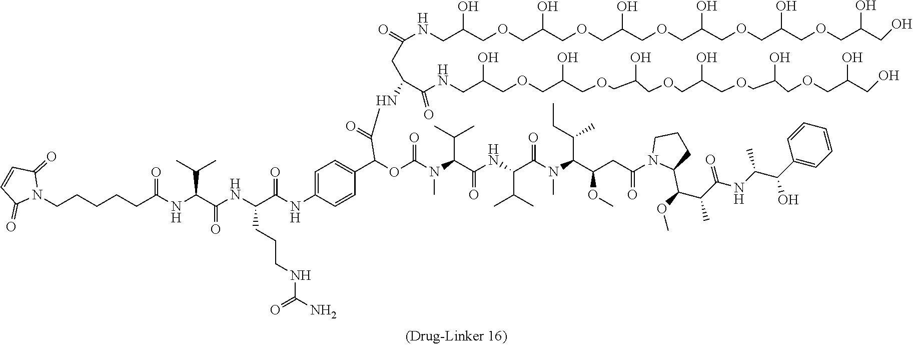 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

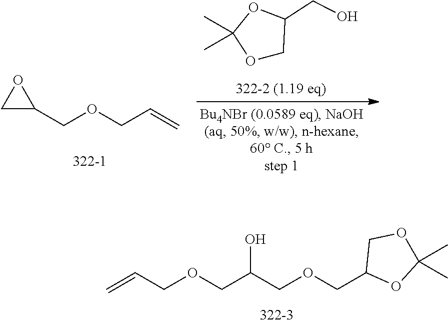

A solution of 322-2 (12.5 g, 94.64 mmol) and Bu₄NBr (1.51 g, 4.684 mmol) in mixture solvent of n-hexane (62.6 mL) and NaOH (aq, 50% w/w, 62.5 mL) was stirred at 60° C. A solution of 322-1 (9.07 g, 79.53 mmol) in n-hexane (12.5 mL) was dropped to the reaction mixture, and the resulting mixture was stirred at 60° C. for 5 hrs until most of 322-1 was consumed by TLC. The mixture was cooled down to r.t. and diluted with water (100 mL), then it was extracted with MTBE (200 mL×2). The organic parts were combined, dried over anhydrous Na₂SO₄, filtered and concentrated to dryness under reduced pressure. The crude product was purified with column chromatography (silica, 0-50% Ethyl acetate in petroleum ether) affording 322-3 as a colorless oil 20.68 g. ¹H NMR (400 MHz, CDCl₃) δ 5.93-5.80 (m, 1H), 5.30-5.20 (m, 1H), 5.19-5.12 (m, 1H), 4.30-4.15 (m, 1H), 4.05-3.91 (m, 4H), 3.73-3.66 (m, 1H), 3.58-3.43 (m, 6H), 2.74 (s, 1H), 1.39 (d, J=3.6 Hz, 3H), 1.33 (d, J=2.8 Hz, 3H).

### Step 2

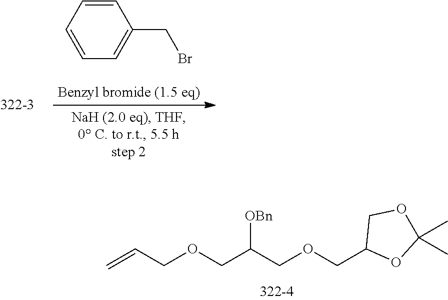 [SMILES: BrCc1ccccc1 ; C=CCOCC(COCC1COC(C)(C)O1)OCc1ccccc1]

To a mixture of NaH (5.58 g, 139.59 mmol) in anhydrous THE (340 mL) cooled in an ice bath was added a solution of 322-3 (17.18 g, 69.79 mmol) in anhydrous THE (85 mL) dropwise, then it was stirred at this temperature for 20 mins. Benzyl bromide (17.79 g, 104.69 mmol) was added dropwise, then the resulting mixture was allowed to warm to r.t. and stirred for 5 hrs until 322-3 was consumed by TLC. The reaction was quenched with saturated ammonium (150 mL) and extracted with ethyl acetate (200 mL×2). The organic phase was combined, dried over anhydrous Na₂SO₄, filtered and concentrated to dryness under reduced pressure to give the crude product, which was purified with column chromatography (silica, 0-40% ethyl acetate in petroleum ether) affording 322-4 (14.7 g, 43.73 mmol, 62.7%) as a colorless oil. Purity=90%-95%. ¹H NMR (400 MHz, CDCl₃) δ 7.41-7.22 (m, 5H), 5.97-5.81 (m, 1H), 5.27 (dt, J=17.2, 1.8 Hz, 1H), 5.17 (dq, J=10.5, 1.5 Hz, 1H), 4.75-4.62 (m, 2H), 4.24 (t, J=6.0 Hz, 1H), 4.07-3.95 (m, 3H), 3.79-3.70 (m, 2H), 3.68-3.59 (m, 2H), 3.58-3.52 (m, 3H), 3.51-3.45 (m, 1H), 1.41 (s, 3H), 1.35 (s, 3H).

### Step 3

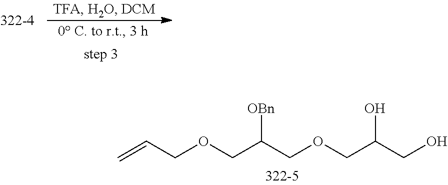 [SMILES: C=CCOCC(COCC(O)CO)OCc1ccccc1]

A solution of 322-4 (3.5 g, 10.411 mmol) in DCM (62.3 mL) and H₂O (0.78 mL) was stirred in an ice bath, then trifluoroacetic acid (1.56 mL) was added. The resulting solution was stirred for 3 hrs at this temperature until 322-4 was consumed by TLC. The reaction was quenched with saturated NaHCO₃ (aq) and then diluted with water (20 mL). The mixture was extracted with DCM (30 mL×2) and the organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-5 (2.815 g, 9.505 mmol, 91.3%) as a colorless oil. Purity=90%-95%. H NMR (400 MHz, CDCl₃) δ 7.40-7.26 (m, 5H), 5.95-5.81 (m, 1H), 5.31-5.23 (m, 1H), 5.21-5.14 (m, 1H), 4.73-4.59 (m, 2H), 3.99 (dt, J=5.6, 1.5 Hz, 2H), 3.85-3.78 (m, 1H), 3.76-3.71 (m, 1H), 3.66-3.49 (m, 8H), 2.55 (s, 2H).

### Step 4

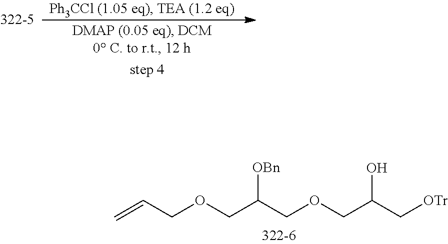

A solution of 322-5 (8.17 g, 27.586 mmol), TEA (3.349 g, 33.103 mmol) and DMAP (168.5 mg, 1.379 mmol) in DCM (40.8 mL) was stirred in an ice bath, then a solution of Ph₃CCl (8.075 g, 28.966 mmol) in DCM (40.8 mL) was added dropwise. The resulting mixture was gradually warmed to room temperature and stirred at ambient temperature for 12 hrs until most of 322-5 was consumed by TLC. The mixture was diluted with DCM (20 mL), washed with water (20 mL×2), dried over anhydrous Na₂SO₄, filtered and concentrated to dryness under reduced pressure to give the crude product, which was purified with column chromatography (silica, 0-40% Ethyl acetate in petroleum ether) affording 322-6 (13.86 g, 25.749 mmol, 93.3%) as a pale yellow oil. Purity=90%-95%. H NMR (400 MHz, CDCl₃) δ 7.47-7.36 (m, 6H), 7.32-7.20 (m, 14H), 5.94-5.90 (m, 1H), 5.29-5.21 (m, 1H), 5.16 (dd, J=10.4, 1.6 Hz, 1H), 4.69-4.57 (m, 2H), 4.0-3.86 (m, 3H), 3.73-3.67 (m, 1H), 3.63-3.55 (m, 3H), 3.55-3.48 (m, 3H), 3.24-3.12 (m, 2H), 2.61 (s, 1H).

### Step 5

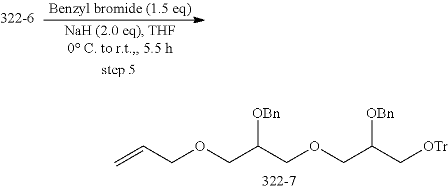

A mixture of NaH (2.06 g, 51.498 mmol) in anhydrous THE (90 mL) was stirred in an ice bath, then a solution of 322-6 (13.86 g, 25.749 mmol) in anhydrous THE (45 mL) was added dropwise. The reaction mixture was stirred at this temperature for 20 mins, then benzyl bromide (6.56 g, 38.62 mmol) was added. The resulting mixture was allowed to warm to r.t. and stirred for 5 hrs until 322-6 was consumed by TLC. The reaction was quenched with saturated ammonium (50 mL) and extracted with (ethyl acetate 60 mL×2). The organic phase was combined, dried over anhydrous Na₂SO₄, filtered and concentrated to dryness under reduced pressure to give the crude product, which was purified with column chromatography (silica, 0-30% ethyl acetate in petroleum ether) affording 322-7 (15.67 g, 24.940 mmol, 96.86%) as a colorless oil. Purity=90%-95%. ¹H NMR (400 MHz, CDCl₃) δ 7.51-7.40 (m, 6H), 7.35-7.20 (m, 19H), 5.94-5.80 (m, 1H), 5.29-5.21 (m, 1H), 5.17-5.10 (m, 1H), 4.65 (d, J=12.1 Hz, 4H), 3.99-3.87 (m, 2H), 3.79-3.65 (m, 2H), 3.65-3.59 (m, 2H), 3.58-3.53 (m, 2H), 3.52-3.46 (m, 2H), 3.23 (d, J=5.0 Hz, 2H).

### Step 6

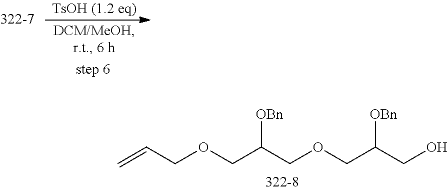 [SMILES: C=CCOCC(COCC(CO)OCc1ccccc1)OCc1ccccc1]

To a solution of 322-7 (15.67 g, 24.939 mmol) in a mixture solvent of DCM (62 mL) and MeOH (31 mL) was added TsOH·H₂O (5.7 g, 29.927 mmol), the resulting mixture was stirred for 6 hrs at room temperature until 322-7 was consumed by TLC. The reaction was quenched with saturated NaHCO₃ (aq.), diluted with water (30 mL), and then extracted with DCM (50 mL×3). The organic phase was combined, dried over anhydrous Na₂SO₄, filtered and concentrated to dryness under reduced pressure to give the crude product, which was purified with column chromatography (silica, 0-50% ethyl acetate in petroleum ether) affording 322-8 (9.09 g, 23.54 mmol, 94.37%) as a pale yellow oil. Purity=90%-95%. ¹H NMR (400 MHz, CDCl₃) δ 7.48-7.15 (m, 1OH), 5.98-5.80 (m, 1H), 5.26 (d, J=17.2 Hz, 1H), 5.17 (d, J=10.4 Hz, 1H), 4.75-4.53 (m, 4H), 3.99 (d, J=5.6 Hz, 2H), 3.78-3.70 (m, 2H), 3.68-3.48 (m, 8H).

### Step 7

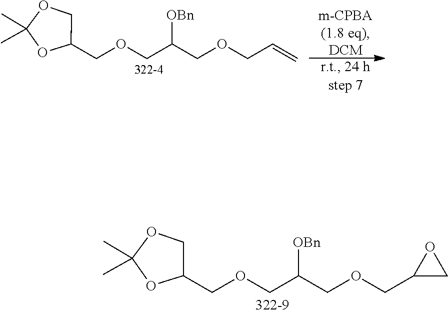

To a solution of 322-4 (14.0 g, 41.555 mmol) in DCM (36 mL) was added a solution of m-CPBA (12.91 g, 74.799 mmol) in DCM (103 mL) dropwise at room temperature. The resulting mixture was stirred at this temperature for 24 hours until 322-4 was consumed, and the reaction was quenched with saturated Na₂S₂O₃ (aq., 50 mL) and NaHCO₃ (aq, 50 mL). The reaction mixture was stirred for 30 mins, and then diluted with DCM (150 mL). The organic phase was washed with a mixture solution (60 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-60% Ethyl acetate in petroleum ether) affording 322-9 (13.79 g, 39.16 mmol, 94.37%) as a pale yellow oil. Purity=90%-95%. ¹H NMR (400 MHz, CDCl₃) δ 7.45-7.16 (m, 5H), 4.69 (s, 2H), 4.25 (t, J=5.6 Hz, 1H), 4.04 (t, J=7.2 Hz, 1H), 3.86-3.69 (m, 3H), 3.68-3.36 (m, 7H), 3.19-3.08 (m, 1H), 2.83-2.76 (m, 1H), 2.63-2.53 (m, 1H), 1.42 (s, 3H), 1.37 (s, 3H).

### Step 8

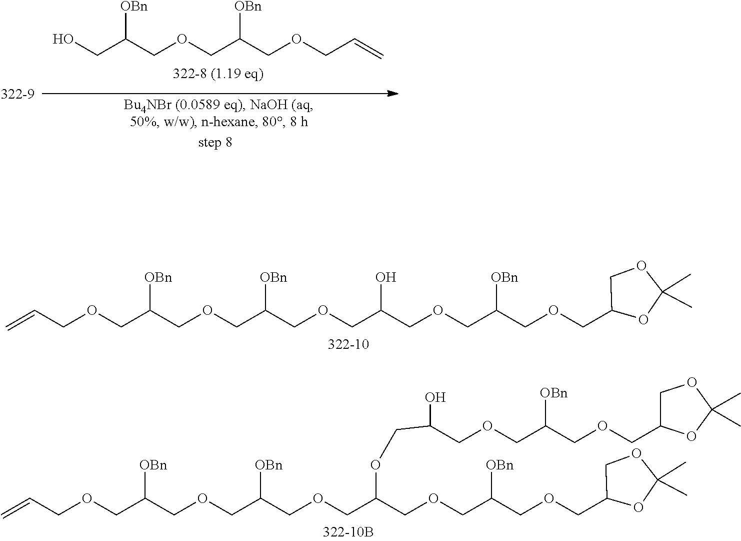

A solution of 322-8 (8.429 g, 23.934 mmol) and Bu₄NBr (454.4 mg, 1.409 mmol) in mixture solvent of n-hexane (30 mL) and NaOH (aq, 50% w/w, 30 mL) was stirred at 80° C. A mixture of 322-9 (11.0 g, 28.482 mmol) in n-hexane (6 mL) was dropped to the reaction mixture, and the resulting mixture was stirred at 80° C. for 8 hrs until most of 322-9 was consumed by TLC. The mixture was cooled down to r.t. and diluted with water (50 mL), then it was extracted with MTBE (100 mL×3). The organic parts were combined, dried over anhydrous Na₂SO₄, filtered and concentrated to dryness under reduced pressure. The crude product was purified with column chromatography (silica, 0-100% ethyl acetate in petroleum ether) affording 322-10 (8.0 g, 10.83 mmol, 45.3%) as a pale yellow oil and 322-10B (2.1 g, 1.925 mmol, 8.0%) as a pale yellow oil. ¹H NMR (400 MHz, CDCl₃) δ 7.38-7.15 (m, 15H), 5.96-5.73 (m, 1H), 5.31-5.06 (m, 2H), 4.76-4.51 (m, 6H), 4.29-4.13 (m, 1H), 4.07-3.86 (m, 4H), 3.76-3.37 (m, 22H), 2.52 (s, 1H), 1.56-1.05 (m, 6H). ¹H NMR (400 MHz, CDCl₃) δ 7.42-7.27 (m, 20H), 5.96-5.82 (m, 1H), 5.26 (dd, J=17.2, 1.8 Hz, 1H), 5.16 (dd, J=10.5, 1.9 Hz, 1H), 4.69-4.61 (m, 8H), 4.27-4.19 (m, 2H), 4.05-3.97 (m, 4H), 3.90-3.80 (m, 2H), 3.76-3.66 (m, 8H), 3.61-3.48 (m, 26H), 3.09 (s, 1H), 1.58-1.16 (m, 12H).

### Step 9

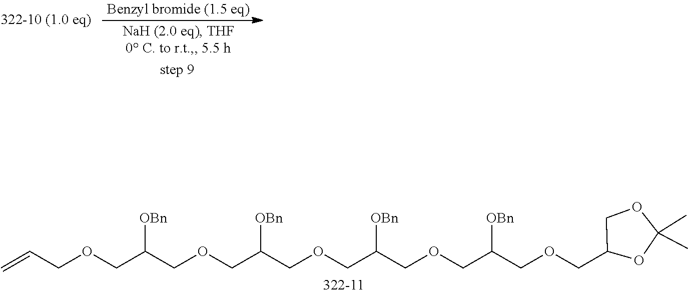 [SMILES: C=CCOCC(COCC(COCC(COCC(COCC1COC(C)(C)O1)OCc1ccccc1)OCc1ccccc1)OCc1ccccc1)OCc1ccccc1]

A mixture of NaH (867 mg, 21.668 mmol) in anhydrous THE (36 mL) was stirred in an ice bath, then a solution of 322-10 (8.0 g, 10.834 mmol) in anhydrous THE (20 mL) was added dropwise. The reaction mixture was stirred at this temperature for 20 mins, then benzyl bromide (2.76 g, 16.251 mmol) was added. The resulting mixture was allowed to warm to r.t. and stirred for 5 hrs until 322-10 was consumed by TLC. The reaction was quenched with saturated ammonium (25 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-60% Ethyl acetate in petroleum ether) affording 322-11 (8.45 g, 10.20 mmol, 94.1%) as a pale yellow oil. Purity=90%-95%. H NMR (400 MHz, CDCl₃) δ 7.43-7.25 (m, 20H), 5.99-5.82 (m, 1H), 5.27 (dd, J=17.2, 1.8 Hz, 1H), 5.17 (dd, J=10.4, 1.7 Hz, 1H), 4.77-4.60 (m, 8H), 4.30-4.18 (m, 1H), 4.06-3.96 (m, 3H), 3.81-3.70 (m, 5H), 3.63-3.47 (m, 18H), 1.47-1.21 (m, 6H).

### Step 10

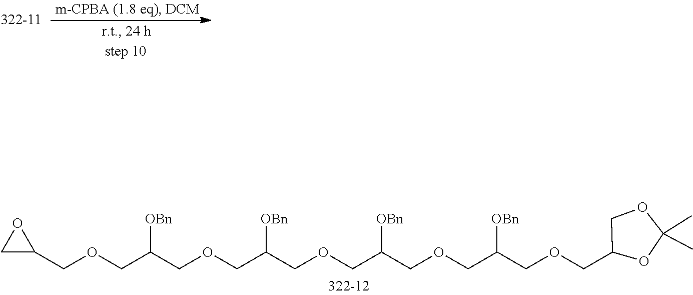 [SMILES: CC1(C)OCC(COCC(COCC(COCC(COCC(COCC2CO2)OCc2ccccc2)OCc2ccccc2)OCc2ccccc2)OCc2ccccc2)O1]

To a solution of 322-11 (8.45 g, 10.20 mmol) in DCM (12.8 mL) was added a solution of m-CPBA (3.16 g, 18.36 mmol) in DCM (25.6 mL) dropwise at room temperature. The resulting mixture was stirred at this temperature for 24 hours until 322-11 was consumed, and the reaction was quenched with saturated Na₂S₂O₃ (aq, 15 mL) and NaHCO₃ (aq, 15 mL). The reaction mixture was stirred for 30 mins, and then diluted with DCM (120 mL). The organic phase was washed with a mixture solution (40 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-60% ethyl acetate in petroleum ether) affording 322-12 (6.858 g, 8.12 mmol, 79.6%) as a pale yellow oil. Purity=90%-95%. H NMR (400 MHz, CDCl₃) δ 7.53-7.25 (m, 20H), 4.68 (s, 8H), 4.28-4.20 (m, 1H), 4.06-4.00 (m, 1H), 3.80-3.69 (m, 6H), 3.66-3.50 (m, 16H), 3.49-3.44 (m, 1H), 3.41-3.35 (m, 1H), 3.16-3.08 (m, 1H), 2.76 (t, J=4.6 Hz, 1H), 2.63-2.52 (m, 1H), 1.54-1.24 (m, 6H).

### Step 11

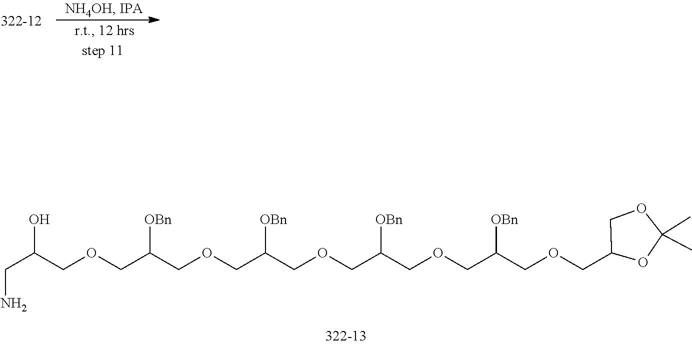 [SMILES: CC1(C)OCC(COCC(COCC(COCC(COCC(COCC(O)CN)OCc2ccccc2)OCc2ccccc2)OCc2ccccc2)OCc2ccccc2)O1]

To a solution of 322-12 (1.22 g, 1.445 mmol) in isopropyl alcohol (120 mL) was added ammonia (120 mL) dropwise at room temperature, and the resulting mixture was stirred at this temperature for 12 hours until 322-12 was consumed. The reaction mixture was concentrated to dryness under reduced pressure to afford 322-13 (1.28 g, 1.48 mmol, 100%) as a colorless oil, which was used in the next step without further purification. Purity=90%-95%.

### Step 12

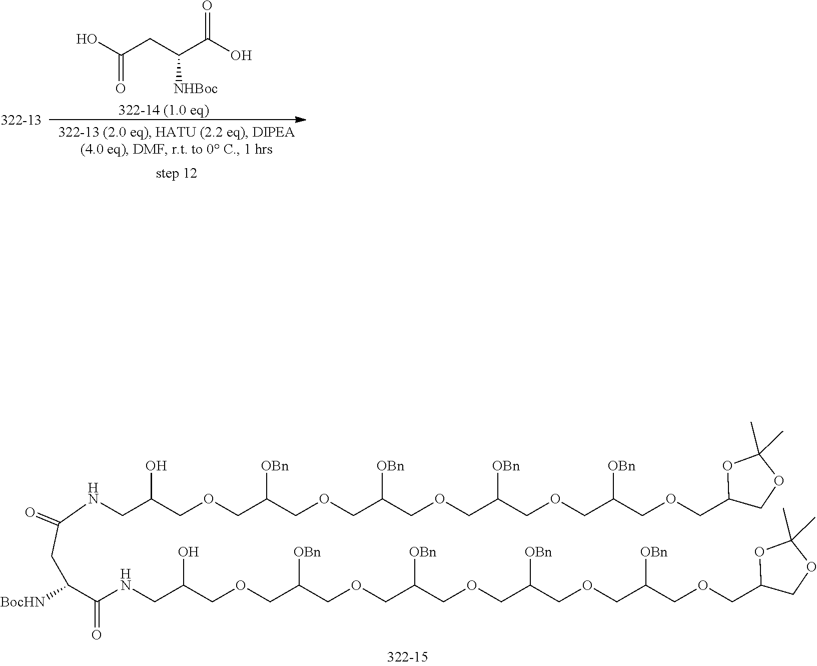 [SMILES: CC(C)(C)OC(=O)N[C@H](CC(=O)O)C(=O)O ; CC(C)(C)OC(=O)N[C@H](CC(=O)NCC(O)COCC(COCC(COCC(COCC(COCC1COC(C)(C)O1)OCc1ccccc1)OCc1ccccc1)OCc1ccccc1)OCc1ccccc1)C(=O)NCC(O)COCC(COCC(COCC(COCC(COCC1COC(C)(C)O1)OCc1ccccc1)OCc1ccccc1)OCc1ccccc1)OCc1ccccc1]

A solution of 322-14 (81.2 mg, 0.348 mmol) and HATU (291 mg, 0.766 mmol) in anhydrous DMF (5 mL) was stirred at room temperature for 15 mins, then it was stirred in an ice bath. A solution of 322-13 (600 mg, 0.696 mmol) in anhydrous DMF (3 mL) was added dropwise, followed by DIPEA (180 mg, 1.392 mmol). The resulting mixture was stirred in the ice bath for 1 h until most of 322-14 was consumed, and then diluted with ethyl acetate (60 mL). The organic phase was washed with H₂O (20 mL×3), dried over anhydrous Na₂SO₄, filtered and concentrated to dryness under reduced pressure to give the crude product, which was purified with column chromatography (silica, 0-100% ethyl acetate in petroleum ether) affording 322-15 (660 mg, 0.343 mmol, 98.7%) as a pale yellow oil. Purity=90%-95%. ¹H NMR (400 MHz, CDCl₃) δ 7.74-7.24 (m, 40H), 6.68-6.40 (m, 1H), 6.25-6.00 (m, 1H), 4.84-4.50 (m, 16H), 4.40 (s, 1H), 4.23 (t, J=6.0 Hz, 2H), 4.05-3.97 (m, 2H), 3.82-3.36 (m, 53H), 3.22-2.96 (m, 3H), 2.93-2.61 (m, 5H), 2.52-2.33 (m, 1H), 1.44-1.26 (m, 21H).

### Step 13

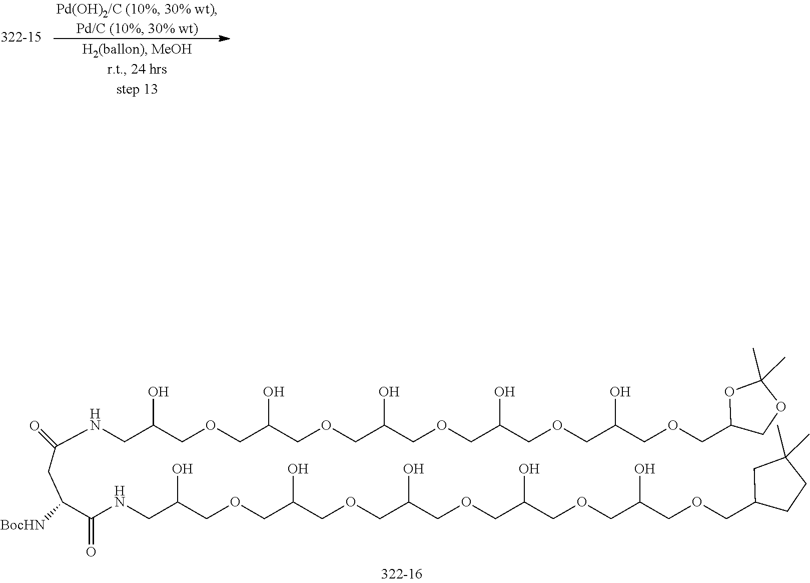 [SMILES: CC1(C)CCC(COCC(O)COCC(O)COCC(O)COCC(O)COCC(O)CNC(=O)[C@@H](CC(=O)NCC(O)COCC(O)COCC(O)COCC(O)COCC(O)COCC2COC(C)(C)O2)NC(=O)OC(C)(C)C)C1]

A mixture of 322-15 (160 mg, 0.0833 mmol), Pd(OH)₂/C (10%, 50 mg) and Pd/C (10%, 50 mg) in MeOH (10 mL) was stirred under hydrogen atmosphere (ballon) for 24 h at room temperature until 322-15 was completely converted into 322-16. The reaction mixture was filtered through a celite pad, and the filtrate was concentrated to dryness under reduced pressure to afford 322-16 (78.3 mg, 0.0652 mmol, 78.3%) as a colorless oil, which was used in the next step without further purification.

### Step 14

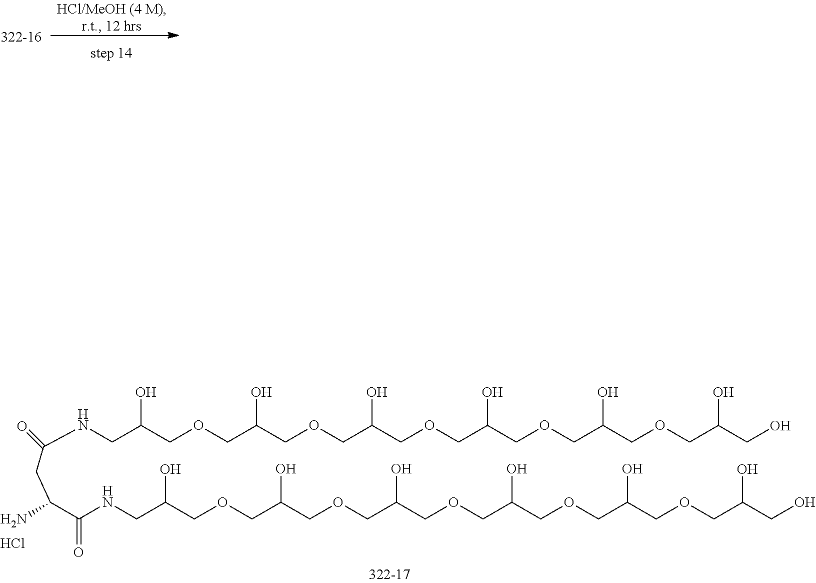 [SMILES: Cl ; N[C@H](CC(=O)NCC(O)COCC(O)COCC(O)COCC(O)COCC(O)COCC(O)CO)C(=O)NCC(O)COCC(O)COCC(O)COCC(O)COCC(O)COCC(O)CO]

A mixture of 322-16 (78.3 mg, 0.0652 mmol) and HCl/MeOH (4M, 4 mL) was stirred at room temperature for 12 hours until 322-16 was completely converted into 322-17. The reaction mixture was concentrated to dryness under reduced pressure to afford 322-17 (65.0 mg, 0.0616 mmol, 94.4%) as an off-white solid, which was used in the next step without further purification.

### Step 15

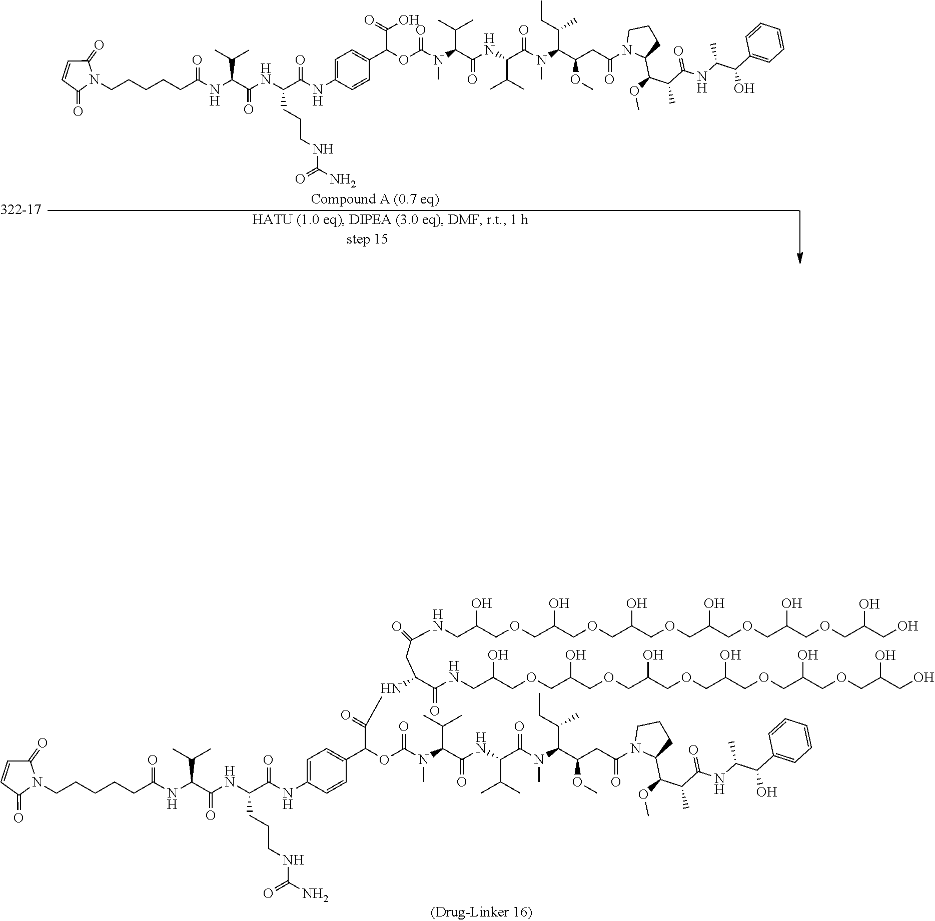

A solution of 322-17 (60 mg, 0.0568 mmol), Compound A (54 mg, 0.0397 mmol), HATU (21.6 mg, 0.0568 mmol) and DIPEA (22 mg, 0.170 mmol) in anhydrous DMF (4 mL) was stirred at room temperature for 1 h until Compound A was consumed.

Then the reaction solution was purified by prep-HPLC to give drug-linker 16 (18.1 mg, 0.00766 mmol, 19.3%) as a white solid. LCMS, m/z=1182.02 (M/2+H)⁺, m/z=788.25 (M/3+H)⁺.

### Example 17: Preparation of Drug-Linker 17

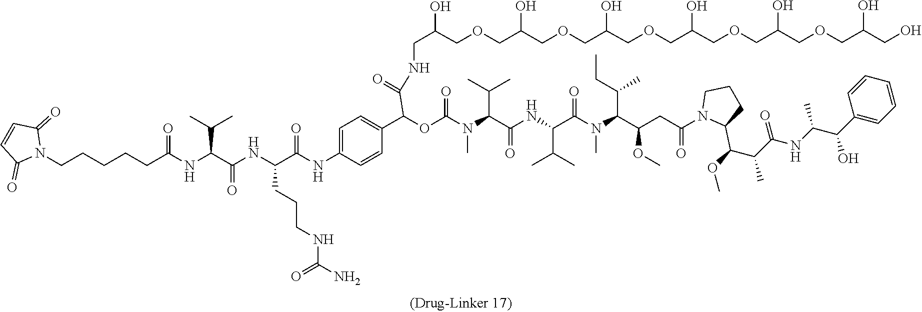 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

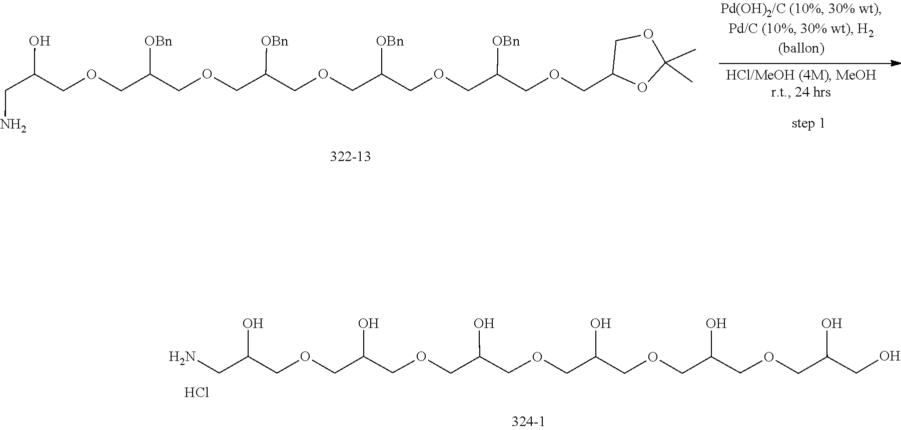 [SMILES: CC1(C)OCC(COCC(COCC(COCC(COCC(COCC(O)CN)OCc2ccccc2)OCc2ccccc2)OCc2ccccc2)OCc2ccccc2)O1 ; NCC(O)COCC(O)COCC(O)COCC(O)COCC(O)COCC(O)CO ; Cl]

A mixture of 322-13 (160 mg, 0.186 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 322-13 was completely converted into 324-1. The reaction mixture was filtered through a celite pad, and the filtrate was concentrated to dryness under reduced pressure to afford 324-1 (92.3 mg, 0.186 mmol, 100%) as an off-white solid, which was used in the next step without further purification.

### Step 2

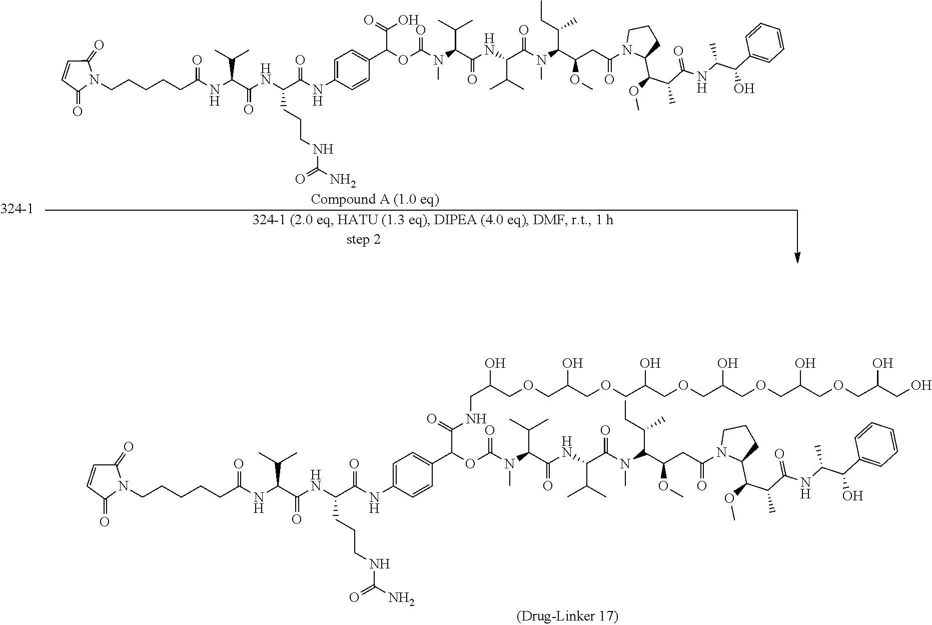

A solution of 324-1 (44 mg, 0.0885 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed.

Then the reaction solution was purified by prep-HPLC to give drug-linker 17 (28.0 mg, 0.0155 mmol, 35.2%) as a white solid. LCMS, m/z=1804.11 (M+H)⁺, m/z=902.76 (M/2+H)⁺.

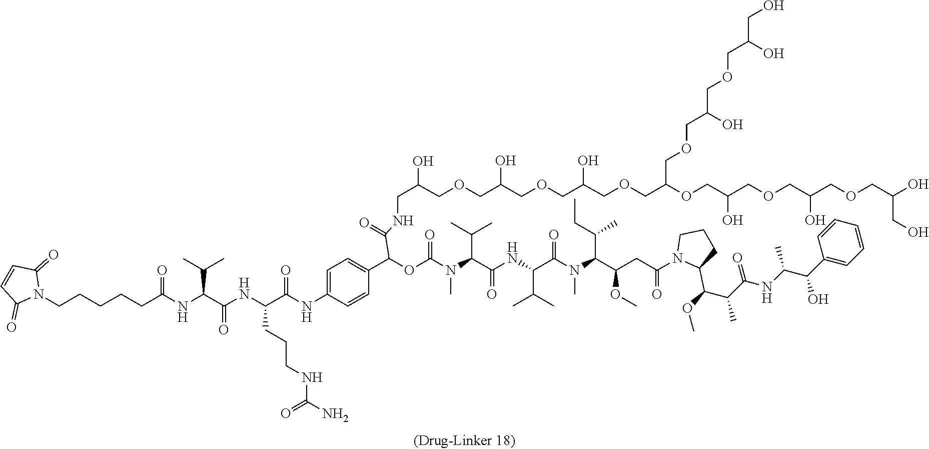 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)NCC(O)COCC(O)COCC(O)COCC(COCC(O)COCC(O)CO)OCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

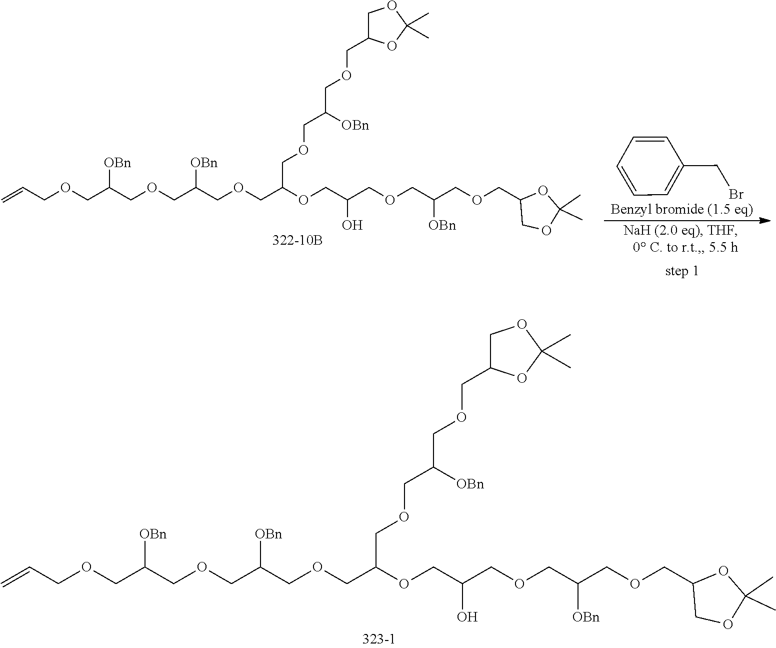

A mixture of NaH (205 mg, 5.13 mmol) in anhydrous THE (12.6 mL) was stirred in an ice bath, then a solution of 322-10B (2.79 g, 2.565 mmol) in anhydrous THE (7 mL) was added dropwise. The reaction mixture was stirred at this temperature for 20 mins, then benzyl bromide (654 g, 3.847 mmol) was added. The resulting mixture was allowed to warm to r.t. and stirred for 5 hrs until 322-10B was consumed by TLC. The reaction was quenched with saturated ammonium (15 mL) and extracted with ethyl acetate (30 mL×2). The organic phase was combined, dried over anhydrous Na₂SO₄, and concentrated to dryness under reduced pressure to give the crude product, which was purified with column chromatography (silica, 0-80% ethyl acetate in petroleum ether) affording 323-1 (2.772 g, 2.348 mmol, 91.8%) as a pale yellow oil. Purity=90%-95%.

### Step 2

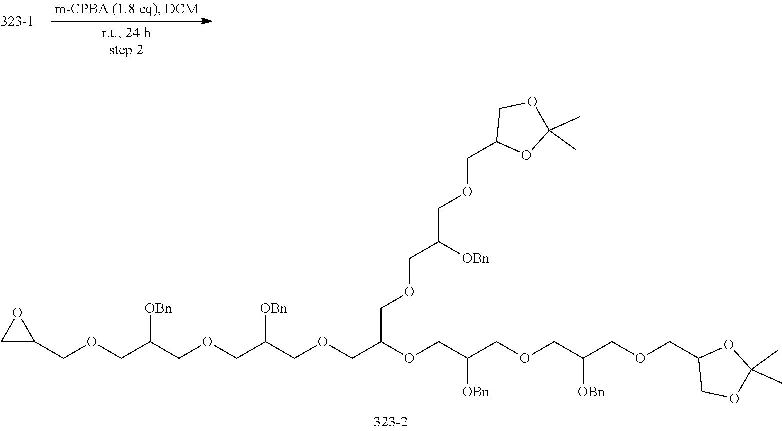 [SMILES: CC1(C)OCC(COCC(COCC(COCC(COCC(COCC2CO2)OCc2ccccc2)OCc2ccccc2)OCC(COCC(COCC2COC(C)(C)O2)OCc2ccccc2)OCc2ccccc2)OCc2ccccc2)O1]

To a solution of 323-1 (2.77 g, 2.346 mmol) in DCM (4.2 mL) was added a solution of m-CPBA (729 mg, 4.223 mmol) in DCM (8.4 mL) dropwise at room temperature. The resulting mixture was stirred at this temperature for 24 hours until 323-1 was consumed, and the reaction was quenched with saturated Na₂S₂O₃ (aq., 10 mL) and NaHCO₃ (aq, 10 mL). The reaction mixture was stirred for 30 mins, and then diluted with DCM (100 mL). The organic phase was washed with a mixture solution (30 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 323-2 (2.147 g, 1.79 mmol, 76.5%) as a pale yellow oil. Purity=90%-95%. ¹H NMR (400 MHz, CDCl₃) δ 7.50-7.20 (m, 25H), 4.65 (s, 1OH), 4.22 (t, J=6.0 Hz, 2H), 4.06-3.98 (m, 2H), 3.82-3.30 (m, 38H), 3.15-3.03 (m, 1H), 2.82-2.70 (m, 1H), 2.62-2.51 (m, 1H), 1.46-1.28 (m, 12H).

### Step 3

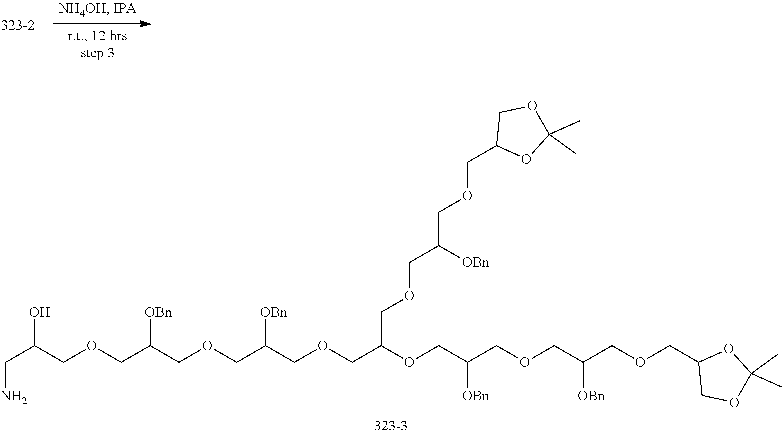 [SMILES: CC1(C)OCC(COCC(COCC(COCC(COCC(COCC(O)CN)OCc2ccccc2)OCc2ccccc2)OCC(COCC(COCC2COC(C)(C)O2)OCc2ccccc2)OCc2ccccc2)OCc2ccccc2)O1]

To a solution of 323-2 (450 mg, 0.376 mmol) in isopropyl alcohol (32 mL) was added ammonia (32 mL) dropwise at room temperature, and the resulting mixture was stirred at this temperature for 12 hours until 323-2 was consumed. The reaction mixture was concentrated to dryness under reduced pressure to afford 323-3 (456 mg, 1.48 mmol, 100%) as a colorless oil, which was used in the next step without further purification. Purity=90%-95%.

### Step 4

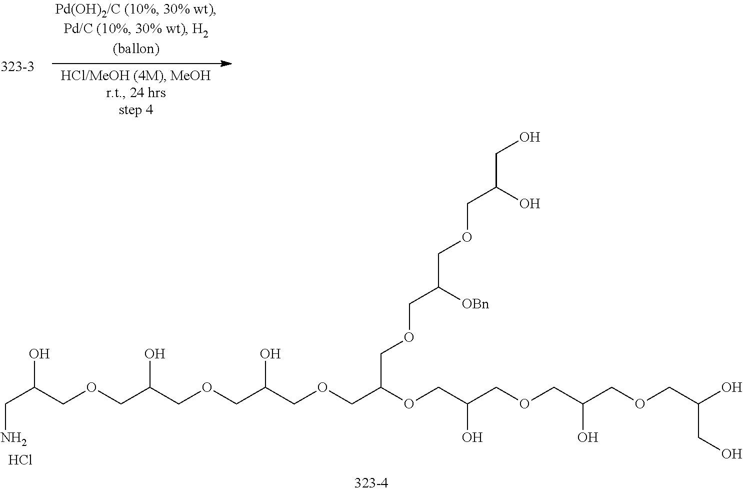 [SMILES: NCC(O)COCC(O)COCC(O)COCC(COCC(COCC(O)CO)OCc1ccccc1)OCC(O)COCC(O)COCC(O)CO ; Cl]

A mixture of 323-3 (250 mg, 0.206 mmol), Pd(OH)₂/C (10%, 75 mg) and Pd/C (10%, 75 mg) in a mixed solvent of MeOH (16 mL) and HCl/MeOH (4M, 4 mL) was stirred under hydrogen atmosphere (ballon) for 24 h at room temperature until 323-3 was completely converted into 323-4. The reaction mixture was filtered through a celite pad, and the filtered was concentrated to dryness under reduced pressure to afford 323-4 (149.7 mg, 0.208 mmol, 100%) as an off-white solid, which was used in the next step without further purification.

### Step 5

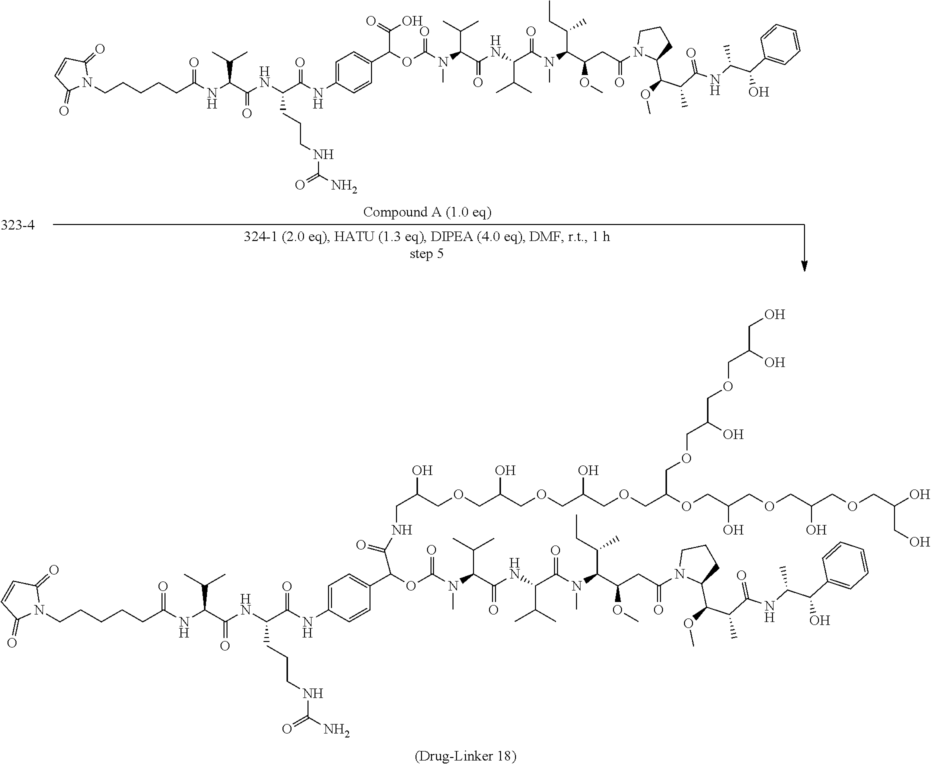

A solution of 323-4 (63.5 mg, 0.0883 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed.

Then the reaction solution was purified by prep-HPLC to give drug-linker 18 (35.0 mg, 0.0173 mmol, 39.2%) as a white solid. LCMS, m/z=1013.85 (M/2+H)⁺.

### Example 19 Preparation of Drug-Linker 19

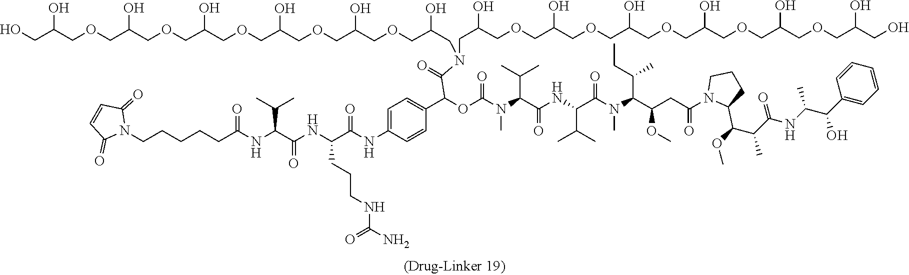 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N(CC(O)COCC(O)COCC(O)COCC(O)COCC(O)COCC(O)CO)CC(O)COCC(O)COCC(O)COCC(O)COCC(O)COCC(O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

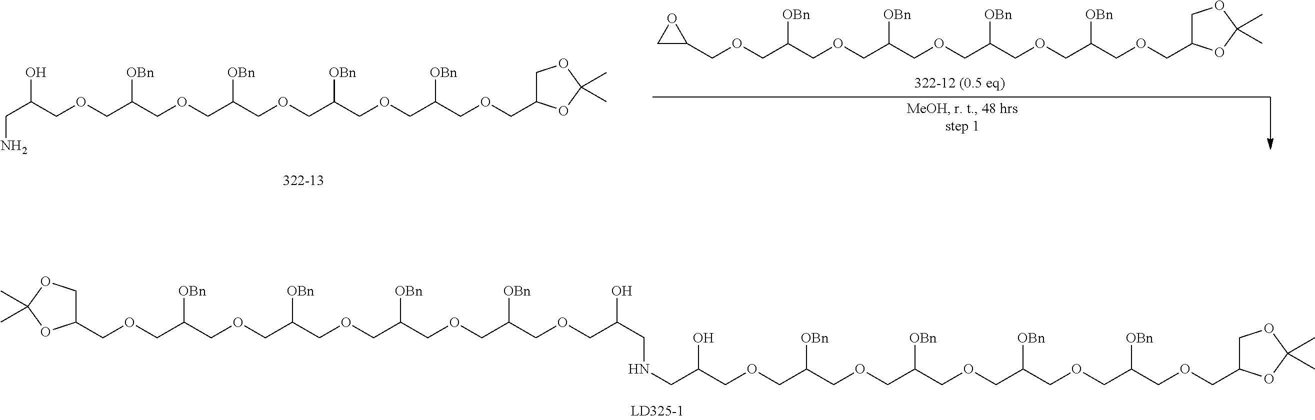

A solution of 322-13 (408 mg, 0.474 mmol), 322-12 (200 mg, 0.237 mmol) and MeOH (20 mL) was stirred at room temperature for 48 hrs. Then the reaction solution was purified by prep-HPLC to afford 325-1 (121.9 mg, 0.0715 mmol, 30.2%) as a colorless oil.

### Step 2

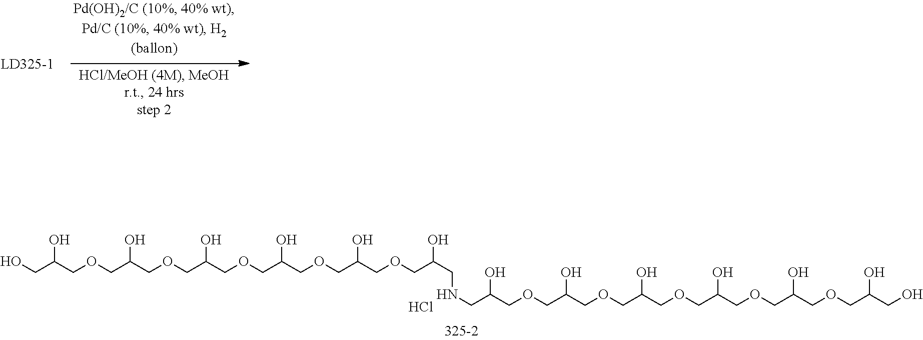 [SMILES: OCC(O)COCC(O)COCC(O)COCC(O)COCC(O)COCC(O)CNCC(O)COCC(O)COCC(O)COCC(O)COCC(O)COCC(O)CO ; Cl]

A mixture of 325-1 (121.9 mg, 0.0715 mmol), Pd(OH)₂/C (10%, 50 mg) and Pd/C (10%, 50 mg) in a mixed solvent of MeOH (10 mL) and HCl/MeOH (4M, 3 mL) was stirred under hydrogen atmosphere (ballon) for 24 h at room temperature until 325-1 was completely converted into 325-2. The reaction mixture was filtered through a celite pad, and the filtrate was concentrated to dryness under reduced pressure to afford 323-4 (67.2 mg, 0.0714 mmol, 100%) as an off-white solid, which was used in the next step without further purification.

### Step 3

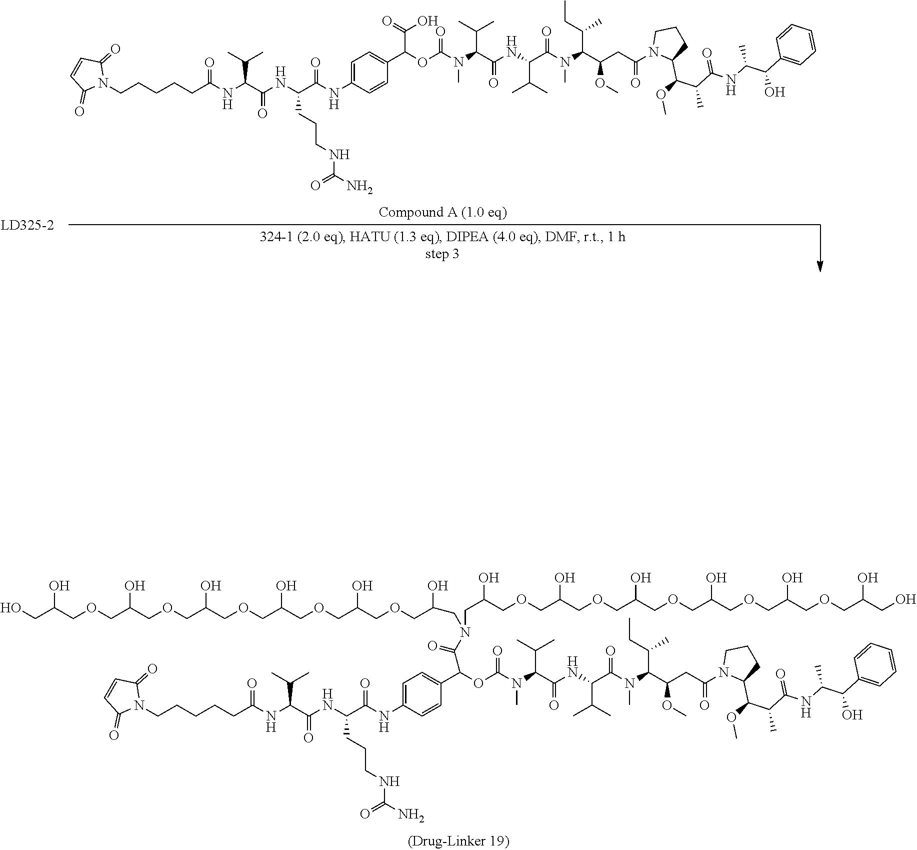

A solution of 325-2 (67.2 mg, 0.0714 mmol), Compound A (60 mg, 0.0441 mmol), HATU (21.8 mg, 0.0573 mmol) and DIPEA (22.8 mg, 0.176 mmol) in anhydrous DMF (4 mL) was stirred at room temperature for 1 h until Compound A was consumed. Then the reaction solution was purified by prep-HPLC to give drug-linker 19 (25.0 mg, 0.0111 mmol, 25.2%) as a white solid. LCMS, m/z=1124.79 (M/2+H)⁺.

### Example 20: Preparation of Drug-Linker 20

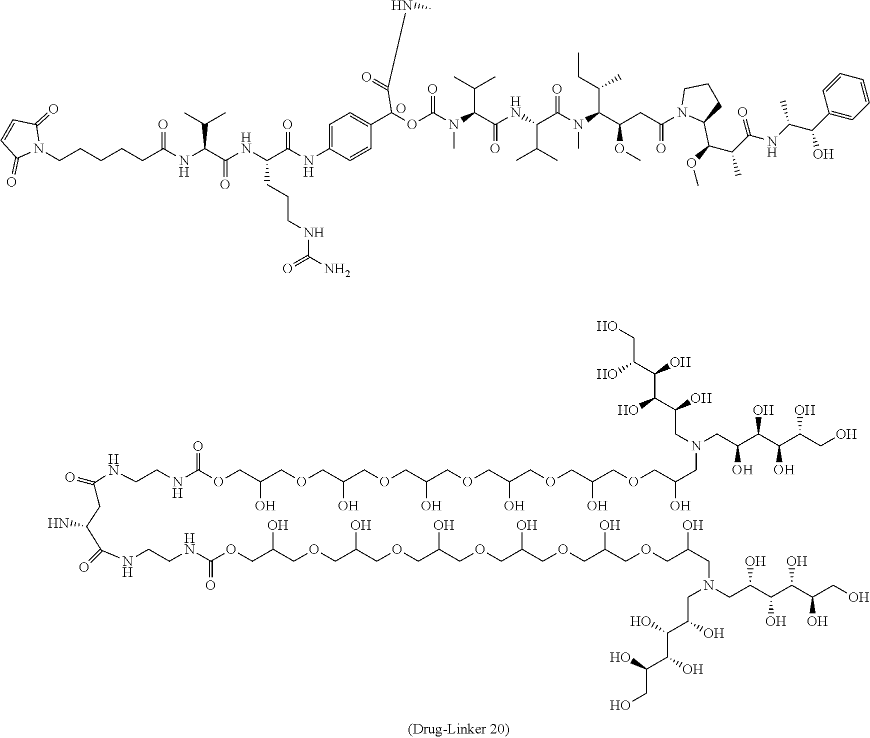

### Step 1

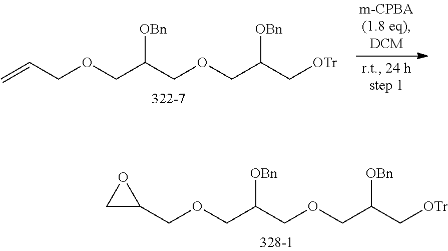

To a solution of 322-7 (10.0 g, 15.915 mmol) in DCM (20 mL) was added a solution of m-CPBA (4.94 g, 28.648 mmol) in DCM (40 mL) dropwise at room temperature. The resulting mixture was stirred at this temperature for 24 hours until 322-7 was consumed, and the reaction was quenched with saturated Na₂S₂O₃ (aq., 20 mL) and NaHCO₃ (aq, 20 mL). The reaction mixture was stirred for 30 mins, and then diluted with DCM (100 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-90% ethyl acetate in petroleum ether) affording 328-1 (9.1 g, 14.12 mmol, 88.7%) as a pale yellow oil. Purity=90%-95%. ¹H NMR (400 MHz, CDCl₃) δ 7.47-7.38 (m, 5H), 7.36-7.03 (m, 20H), 4.70-4.39 (m, 4H), 3.79-3.41 (m, 9H), 3.40-3.32 (m, 1H), 3.28-3.14 (m, 2H), 3.12-3.01 (m, 1H), 2.79-2.66 (m, 1H), 2.60-2.47 (m, 1H).

### Step 2

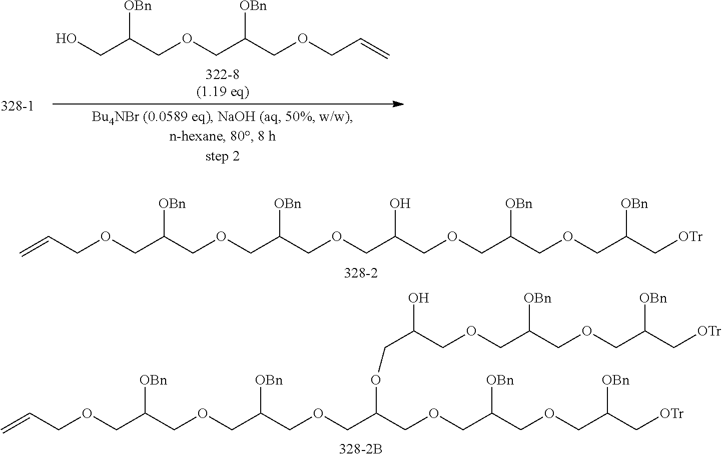

A solution of 322-8 (4.279 g, 11.08 mmol) and Bu₄NBr (177 mg, 0.548 mmol) in mixture solvent of n-hexane (20 mL) and NaOH (aq, 50% w/w, 20 mL) was stirred at 80° C. A mixture of 328-1 (6.0 g, 9.312 mmol) in n-hexane (8 mL) was dropped to the reaction mixture, and the resulting mixture was stirred at 80° C. for 8 hrs until most of 328-1 was consumed by TLC. The mixture was cooled down to r.t. and diluted with water (30 mL), then it was extracted with MTBE (70 mL×3). The organic parts were combined, dried over anhydrous Na₂SO₄, filtered and concentrated to dryness under reduced pressure. The crude product was purified with column chromatography (silica, 0-100% Ethyl acetate in petroleum ether) affording 328-2 (7.22 g, contains 328-2B) as a pale yellow oil. 1H NMR (400 MHz, CDCl₃) δ 7.48-7.41 (m, 6H), 7.37-7.24 (m, 29H), 5.95-5.81 (m, 1H), 5.29-5.22 (m, 1H), 5.16 (dd, J=10.4, 1.7 Hz, 1H), 4.69-4.63 (m, 6H), 4.62-4.58 (m, 2H), 4.00-3.95 (m, 2H), 3.92-3.85 (m, 1H), 3.76-3.70 (m, 3H), 3.69-3.64 (m, 1H), 3.62-3.50 (m, 15H), 3.48-3.39 (m, 4H), 3.23 (d, J=5.2 Hz, 2H).

### Step 3

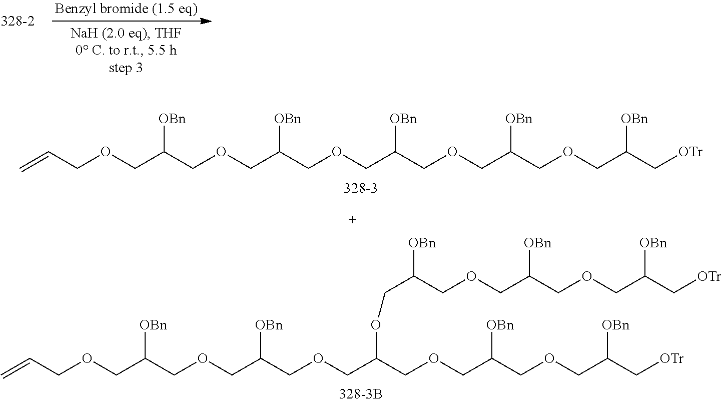

A mixture of NaH (560.5 mg, 14.012 mmol) in anhydrous THE (23.5 mL) was stirred in an ice bath, then a solution of 328-2 (7.22 g, 7.006 mmol, contains 328-2B) in anhydrous THE (13.2 mL) was added dropwise. The reaction mixture was stirred at this temperature for 20 mins, then benzyl bromide (1.786 g, 10.509 mmol) was added. The resulting mixture was allowed to warm to r.t. and stirred for 5 hrs until 328-2 was consumed by TLC. The reaction was quenched with saturated ammonium (20 mL) and extracted with ethyl acetate (40 mL×2). The organic phase was combined, dried over anhydrous Na₂SO₄, filtered and concentrated to dryness under reduced pressure to give the crude product, which was purified with column chromatography (silica, 0-80% Ethyl acetate in petroleum ether) affording 328-3 (5.13 g, 4.578 mmol, 65.3%) as a pale yellow oil and 328-3B (2.0 g, 1.133 mmol) a pale yellow oil. Purity=90%-95%. ¹H NMR (400 MHz, CDCl₃) δ 7.46-7.42 (m, 6H), 7.33-7.20 (m, 34H), 5.94-5.81 (m, 1H), 5.27-5.22 (m, 1H), 5.17-5.12 (m, 1H), 4.67-4.63 (m, 8H), 4.61 (s, 2H), 3.99-3.95 (m, 2H), 3.75-3.66 (m, 5H), 3.60-3.50 (m, 18H), 3.23 (d, J=5.2 Hz, 2H). ¹H NMR (400 MHz, CDCl₃) δ 7.46-7.40 (m, 13H), 7.34-7.25 (m, 27H), 7.24-7.15 (m, 25H), 5.93-5.81 (m, 1H), 5.26-5.21 (m, 1H), 5.14 (dd, J=10.4, 1.6 Hz, 1H), 4.67-4.65 (m, 2H), 4.64-4.61 (m, 8H), 4.60-4.57 (m, 4H), 3.99-3.93 (m, 2H), 3.75-3.63 (m, 1 OH), 3.59-3.55 (m, 6H), 3.54-3.46 (m, 20H), 3.21 (d, J=4.8 Hz, 4H).

### Step 4

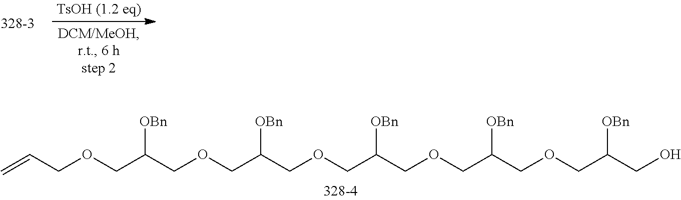 [SMILES: C=CCOCC(COCC(COCC(COCC(COCC(CO)OCc1ccccc1)OCc1ccccc1)OCc1ccccc1)OCc1ccccc1)OCc1ccccc1]

To a solution of 328-3 (3.6 g, 3.213 mmol) in a mixture solvent of DCM (22 mL) and MeOH (11 mL) was added TsOH·H₂O (733 mg, 3.855 mmol), the resulting mixture was stirred for 6 hrs at room temperature until 328-3 was consumed by TLC. The reaction was quenched with saturated NaHCO₃ (aq.), diluted with water (20 mL), and then extracted with DCM (40 mL×3). The organic phase was combined, dried over anhydrous Na₂SO₄, filtered and concentrated to dryness under reduced pressure to give the crude product, which was purified with column chromatography (silica, 0-60% ethyl acetate in petroleum ether) affording 328-4 (2.84 g, 23.233 mmol, 100%) as a colorless oil. Purity=90%-95%. ¹H NMR (400 MHz, CDCl₃) δ 7.38-7.26 (m, 25H), 5.96-5.84 (m, 1H), 5.27 (dq, J=17.2, 1.8 Hz, 1H), 5.17 (dq, J=10.4, 1.6 Hz, 1H), 4.73-4.63 (m, 1 OH), 4.63-4.58 (m, 1H), 3.99 (dt, J=5.6, 1.6 Hz, 2H), 3.78-3.71 (m, 5H), 3.67-3.63 (m, 2H), 3.62-3.54 (m, 18H).

### Step 5

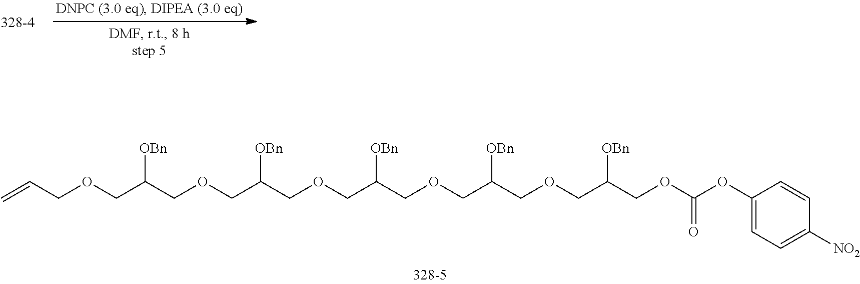 [SMILES: C=CCOCC(COCC(COCC(COCC(COCC(COC(=O)Oc1ccc([N+](=O)[O-])cc1)OCc1ccccc1)OCc1ccccc1)OCc1ccccc1)OCc1ccccc1)OCc1ccccc1]

To a solution of 328-4 (3.6 g, 3.213 mmol) in DMF (8 mL) was added DIPEA (353.2 mg, 2.733 mmol), followed by 4,4′-dinitrodiphenyl carbonate (831.1 mg, 2.732 mmol), then the resulting mixture was stirred at room temperature for 8 hrs until 328-4 was consumed detected by LCMS. The reaction solution was directly used in the next step without work-up procedure.

### Step 6

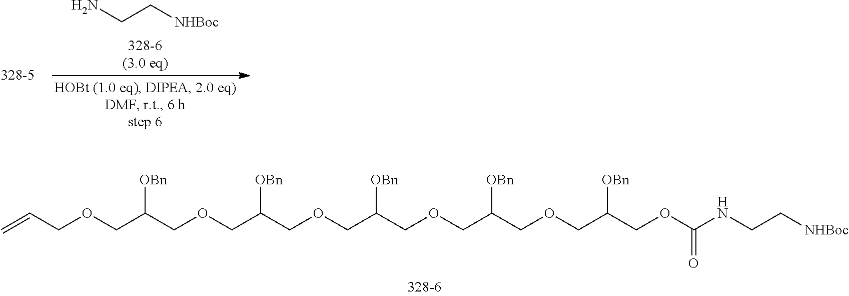 [SMILES: CC(C)(C)OC(=O)NCCN ; C=CCOCC(COCC(COCC(COCC(COCC(COC(=O)NCCNC(=O)OC(C)(C)C)OCc1ccccc1)OCc1ccccc1)OCc1ccccc1)OCc1ccccc1)OCc1ccccc1]

To the above reaction mixture was added HOBt (123 mg, 0.911 mmol), DIPEA (235.5 mg, 1.822 mmol) and 328-6 (437.6 mg, 2.733 mmol) successively, and the resulting mixture was stirred at room temperature for 6 hrs until 328-5 was consumed detected by LCMS. The reaction mixture was diluted with ethyl acetate (100 mL) and washed with saturated NaHCO₃ (aq, 30 mL×3), dried over anhydrous Na₂SO₄, filtered and concentrated to dryness under reduced pressure. The crude product was purified with column chromatography (silica, 0-90% ethyl acetate in petroleum ether) affording 328-6 (958 mg, 0.90 mmol, 98.7% over 2 steps) as a pale yellow oil. ¹H NMR (400 MHz, CDCl₃) δ 7.36-7.22 (m, 25H), 5.95-5.80 (m, 1H), 5.28-5.21 (m, 1H), 5.20-5.13 (m, 1H), 5.07 (s, 1H), 4.83 (s, 1H), 4.69-4.62 (m, 1OH), 4.30-4.21 (m, 1H), 4.16-4.10 (m, 1H), 3.97 (dt, J=5.6, 1.6 Hz, 2H), 3.77-3.69 (m, 5H), 3.62-3.50 (m, 19H), 3.27-3.10 (m, 4H), 1.43 (s, 9H).

### Step 7

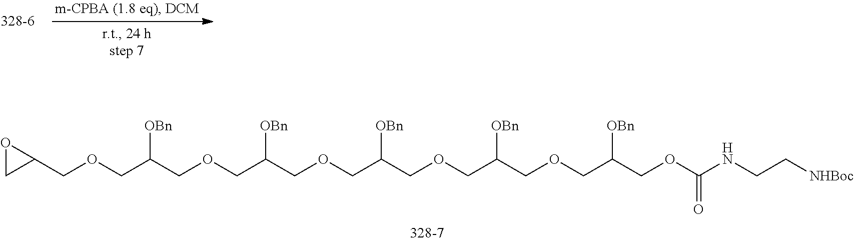 [SMILES: CC(C)(C)OC(=O)NCCNC(=O)OCC(COCC(COCC(COCC(COCC(COCC1CO1)OCc1ccccc1)OCc1ccccc1)OCc1ccccc1)OCc1ccccc1)OCc1ccccc1]

To a solution of 328-6 (958 mg, 0.90 mmol) in DCM (6 mL) was added a solution of m-CPBA (280 mg, 1.62 mmol) in DCM (6 mL) dropwise at room temperature. The resulting mixture was stirred at this temperature for 24 hours until 328-6 was consumed, and the reaction was quenched with saturated Na₂S₂O₃ (aq, 5 mL) and NaHCO₃ (aq., 5 mL). The reaction mixture was stirred for 30 mins, and then diluted with DCM (30 mL). The organic phase was washed with a mixture solution (10 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 328-7 (787 mg, 0.728 mmol, 80.9%) as a pale yellow oil. Purity=90%-95%.

### Step 8

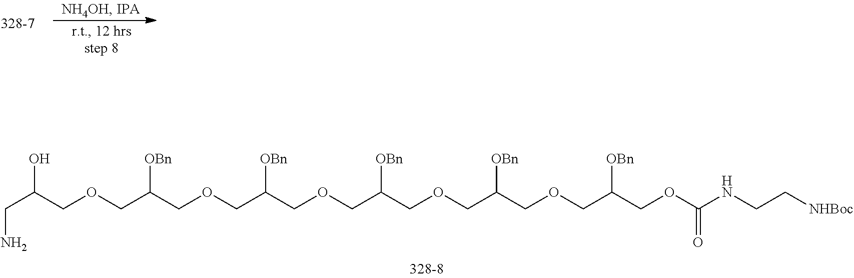 [SMILES: CC(C)(C)OC(=O)NCCNC(=O)OCC(COCC(COCC(COCC(COCC(COCC(O)CN)OCc1ccccc1)OCc1ccccc1)OCc1ccccc1)OCc1ccccc1)OCc1ccccc1]

To a solution of 328-7 (565 mg, 0.523 mmol) in isopropyl alcohol (43 mL) was added ammonia (43 mL) dropwise at room temperature, and the resulting mixture was stirred at this temperature for 12 hours until 328-7 was consumed. The reaction mixture was concentrated to dryness under reduced pressure to afford 328-8 (552.6 mg, 0.503 mmol, 96.3%) as a yellow oil, which was used in the next step without further purification. Purity=90%-95%.

### Step 9

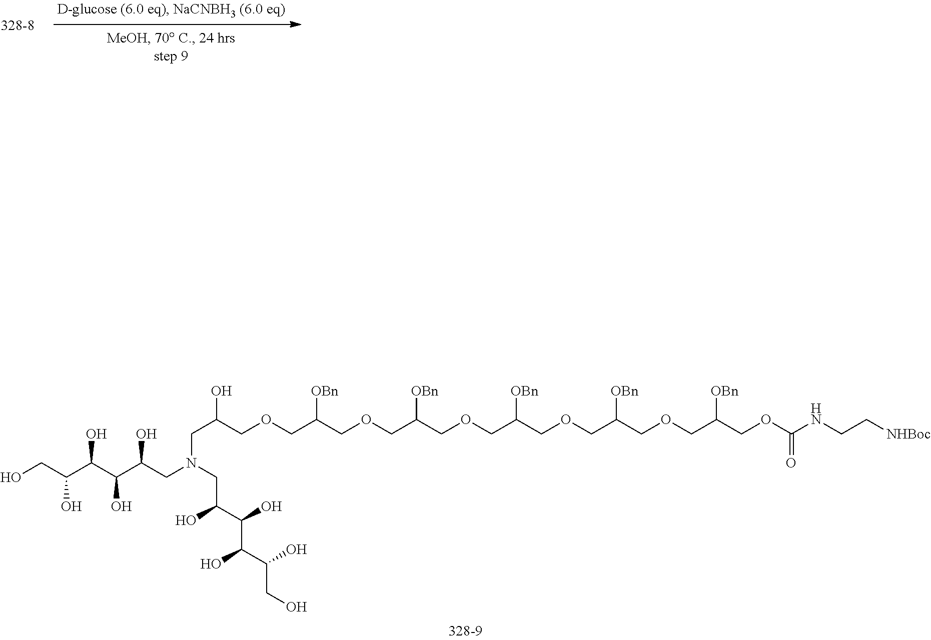 [SMILES: CC(C)(C)OC(=O)NCCNC(=O)OCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 328-8 (552.6 mg, 0.503 mmol), D-glucose (544.2 mg, 3.021 mmol) and NaCNBH₃ (189.8 mg, 3.021 mmol) in anhydrous MeOH (12 mL) was stirred at 70° C. for 24 hrs until most of 328-8 was consumed and 328-9 was detected by LCMS. The reaction mixture was cooled down to room temperature, filtered and concentrated under reduced pressure to give the crude product, which was purified by reverse phase liquid chromatography to give 328-9 (597 mg, 0.419 mmol, 83.2%) as a colorless oil. Purity=90%-95%. ¹H NMR (400 MHz, MeOH-d₄) δ 7.41-7.20 (m, 25H), 4.69-4.57 (m, 1OH), 4.26-4.04 (m, 5H), 3.84-3.78 (m, 3H), 3.77-3.68 (m, 8H), 3.67-3.42 (m, 28H), 3.38-3.34 (m, 1H), 3.33-3.31 (m, 1H), 3.17-3.06 (m, 4H), 1.40 (s, 9H).

### Step 10

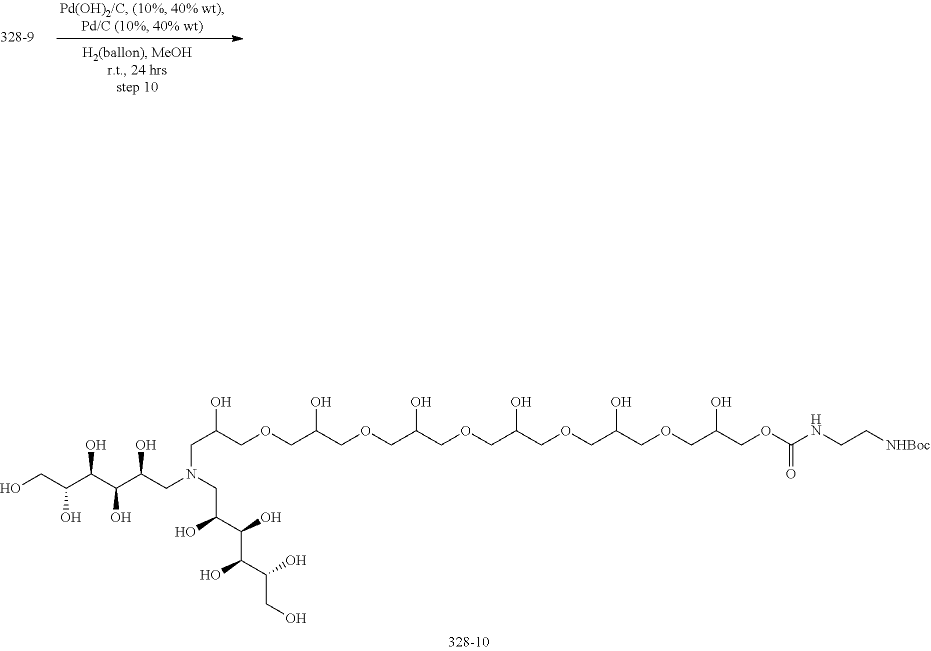 [SMILES: CC(C)(C)OC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 328-9 (376 mg, 0.264 mmol), Pd(OH)₂/C (10%, 150 mg) and Pd/C (10%, 150 mg) in MeOH (30 mL) was stirred under hydrogen atmosphere (ballon) for 24 h at room temperature until 328-9 was completely converted into 328-10. The reaction mixture was filtered through a celite pad, and the filtrate was concentrated to dryness under reduced pressure to afford 328-10 (211.4 mg, 0.217 mmol, 82.1%) as an off-white solid, which was used in the next step without further purification.

### Step 11

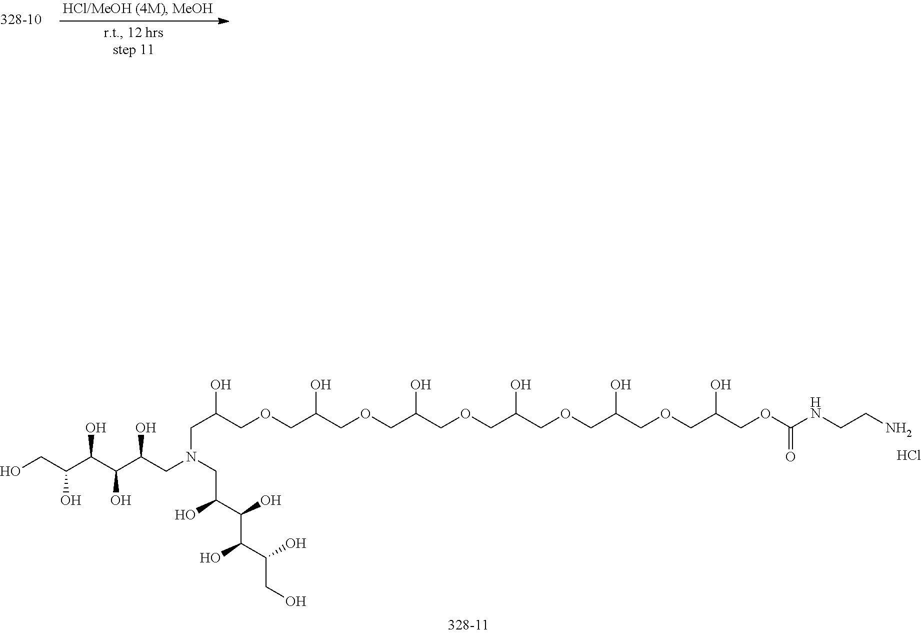 [SMILES: NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 328-10 (211.4 mg, 0.216 mmol) in HCl/MeOH (4M, 3 mL) and MeOH (3 mL) was stirred at room temperature for 12 hours until 328-10 was completely converted into 328-11. The reaction mixture was concentrated to dryness under reduced pressure to afford 328-11 (198.0 mg, 0.217 mmol, 100%) as an off-white solid, which was used in the next step without further purification.

### Step 12

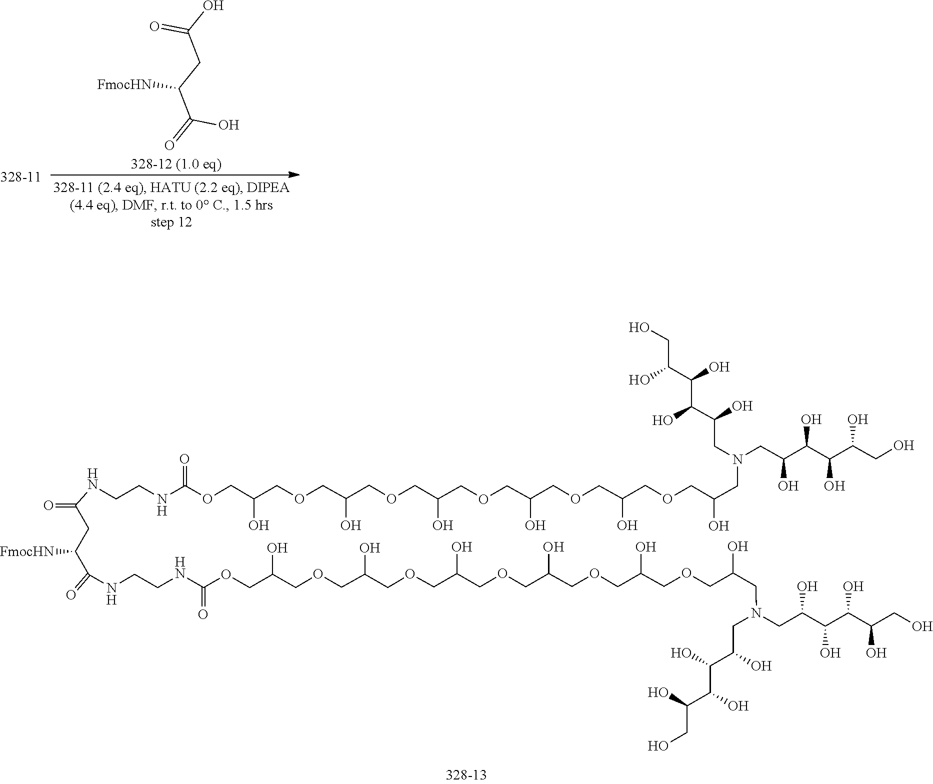

A solution of 328-12 (24.8 mg, 0.0698 mmol) and HATU (58.4 mg, 0.153 mmol) in anhydrous DMF (2 mL) was stirred at room temperature for 15 mins, then it was stirred in an ice bath. A solution of 328-11 (150 mg, 0.168 mmol) in anhydrous DMF (2 mL) was added dropwise, followed by DIPEA (39.7 mg, 0.307 mmol). The resulting mixture was stirred in the ice bath for 1 h until most of 328-12 was consumed. The reaction mixture was purified by reverse phase liquid chromatography to give 328-13 (97.5 mg, 0.0471 mmol, 67.4%) as a colorless oil. Purity=90%-95%.

### Step 13

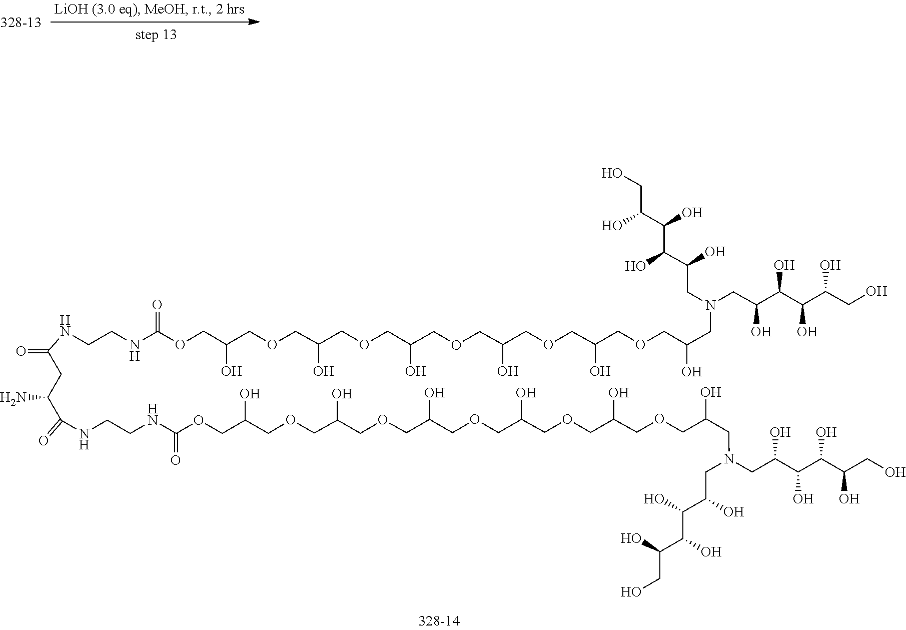 [SMILES: N[C@H](CC(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 328-13 (97.5 mg, 0.0471 mmol) in MeOH (3 mL) was added LiOH·H₂O (5.9 mg, 0.141 mmol), and the mixture was stirred at room temperature for 2 hrs until 328-13 was consumed. The reaction solution was neutralized with 1N HCl to pH=7, and concentrated under reduced pressure to give a crude product, which was dissolved in H₂O (10 mL) and washed with hexane (5 mL×3). The aqueous phase was concentrated to dryness under reduced pressure to afford 328-14 (87.0 mg, 0.047 mmol, 100%) as a colorless oil, which was used in the next step without further purification.

### Step 14

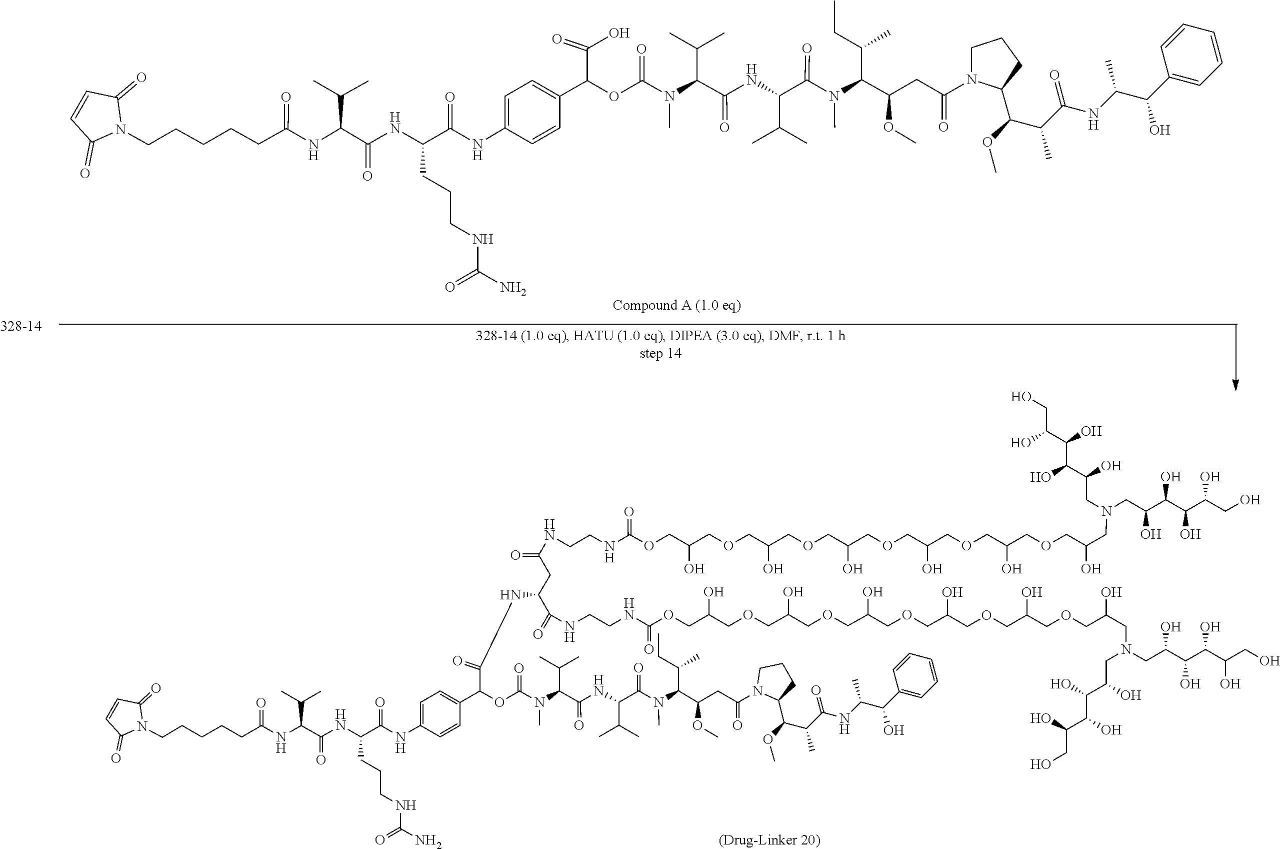

A solution of 328-14 (85 mg, 0.0460 mmol), Compound A (62 mg, 0.0460 mmol), HATU (17.2 mg, 0.0452 mmol) and DIPEA (17.9 mg, 0.139 mmol) in anhydrous DMF (4 mL) was stirred at room temperature for 1 h until Compound A was consumed. Then the reaction solution was purified by prep-HPLC to give drug-linker 20 (73.0 mg, 0.0229 mmol, 49.8%) as a white solid. LCMS, m/z=1595.97 (M/2+H)⁺, m/z=1064.29 (M/2+H)⁺. ¹H NMR (400 MHz, DMSO-d₆) δ 10.04 (s, 1H), 8.59-8.02 (m, 4H), 8.00-7.72 (m, 2H), 7.70-7.46 (m, 3H), 7.45-7.09 (m, 8H), 7.00 (s, 2H), 5.99 (s, 1H), 5.91-5.66 (m, 2H), 5.44 (s, 5H), 5.07-4.29 (m, 16H), 4.25-3.87 (m, 12H), 3.86-3.47 (m, 31H), 3.30-2.74 (m, 27H), 2.44-1.84 (m, 1OH), 1.83-1.63 (m, 4H), 1.59-1.27 (m, 1 OH), 1.24-1.10 (m, 3H), 1.08-0.64 (m, 30H).

### Example 21: Preparation of Drug-Linker 21

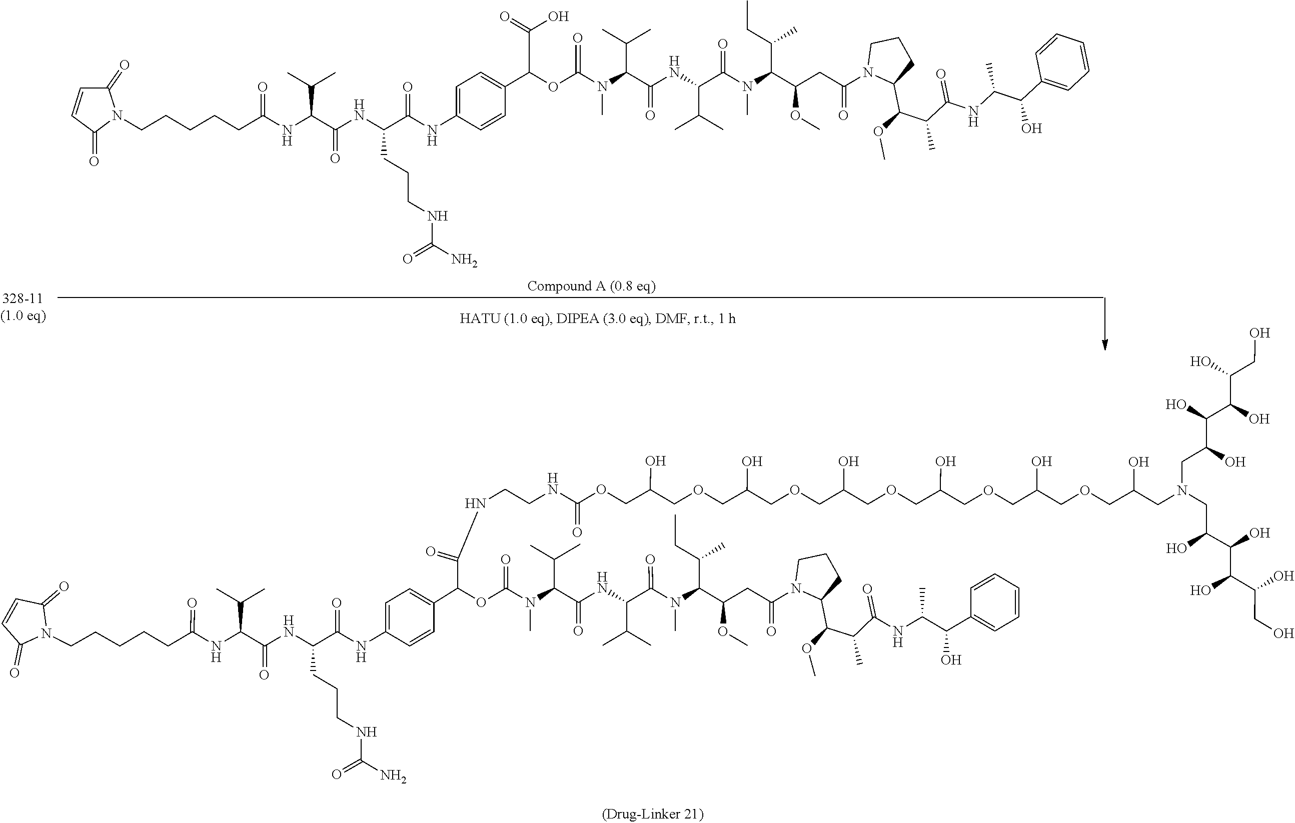

A solution of 328-11 (42 mg, 0.0461 mmol), Compound A (50 mg, 0.0368 mmol), HATU (17.4 mg, 0.0458 mmol) and DIPEA (17.8 mg, 0.138 mmol) in anhydrous DMF (3 mL) was stirred at room temperature for 1 h until Compound A was consumed. Then the reaction solution was purified by prep-HPLC to give drug-linker 21 (37.8 mg, 0.0170 mmol, 46.3%) as a white solid. LCMS, m/z=1109.75 (M/2+H)⁺. ¹H NMR (400 MHz, D₂O) δ 7.60-7.43 (m, 4H), 7.39-7.27 (m, 4H), 7.25-7.14 (m, 1H), 6.78 (s, 2H), 6.05-5.89 (m, 1H), 4.78-4.54 (m, 6H), 4.52-3.90 (m, 28H), 3.89-3.71 (m, 13H), 3.69-3.38 (m, 64H), 3.37-2.85 (m, 25H), 2.81-2.60 (m, 3H), 2.58-2.40 (m, 2H), 2.37-2.11 (m, 4H), 2.08-1.96 (m, 2H), 1.94-1.70 (m, 5H), 1.66-1.45 (m, 8H), 1.35-1.12 (m, 8H), 1.10-0.73 (m, 25H), 0.67-0.50 (m, 1H), 0.47-0.35 (m, 1H).

### Example 22: Preparation of Drug-Linker 22

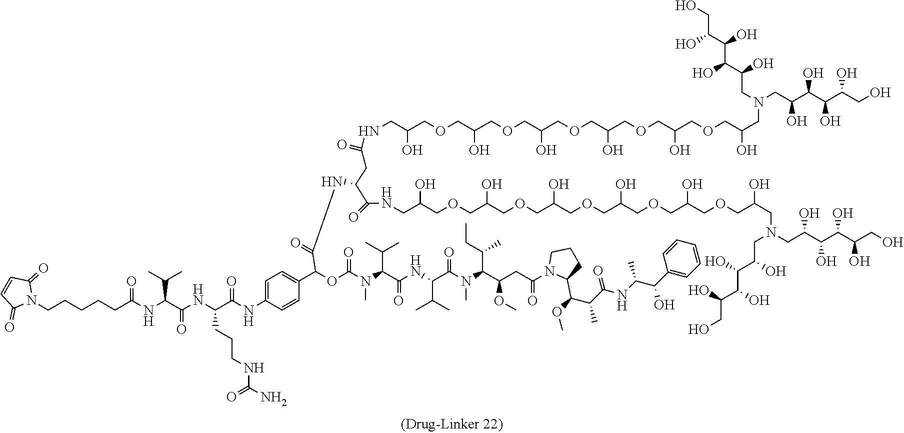 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

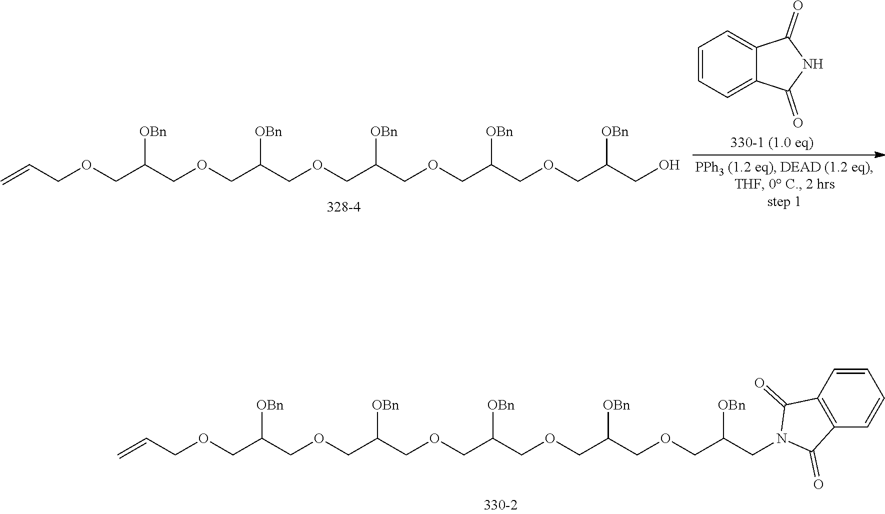 [SMILES: O=C1NC(=O)c2ccccc21 ; C=CCOCC(COCC(COCC(COCC(COCC(CO)OCc1ccccc1)OCc1ccccc1)OCc1ccccc1)OCc1ccccc1)OCc1ccccc1 ; C=CCOCC(COCC(COCC(COCC(COCC(CN1C(=O)c2ccccc2C1=O)OCc1ccccc1)OCc1ccccc1)OCc1ccccc1)OCc1ccccc1)OCc1ccccc1]

To a solution of 330-1 (217.6 mg, 1.48 mmol) and PPh₃ (465.5 mg, 1.776 mmol) in THE (8 mL) was added a solution of 328-4 (1.3 g, 1.48 mmol) in THE (4 mL) and the mixture was stirred in an ice bath. A solution of DEAD (309.3 mg, 1.776 mmol) in THE (1 mL) was added to the above solution and the resulting mixture was allowed to warm to r.t. and stirred for 2 hrs until 328-4 was consumed by TLC. The reaction was quenched with water (1 mL), and the reaction was concentrated under reduced pressure to give the crude product, which was purified with column chromatography (silica, 0-60% ethyl acetate in petroleum ether) to afford 330-2 (1.307 g, 1.297 mmol, 87.7%) as a white solid. Purity=90%-95%. ¹H NMR (400 MHz, CDCl₃) δ 7.77 (dd, J=5.6, 3.2 Hz, 2H), 7.66 (dd, J=5.6, 3.2 Hz, 2H), 7.34-7.20 (m, 20H), 7.19-7.15 (m, 2H), 7.12-7.04 (m, 3H), 5.95-5.81 (m, 1H), 5.29-5.22 (m, 1H), 5.15 (d, J=10.4 Hz, 1H), 4.69-4.63 (m, 9H), 4.51 (d, J=12.0 Hz, 1H), 3.99-3.95 (m, 2H), 3.93-3.83 (m, 2H), 3.76-3.69 (m, 5H), 3.60-3.52 (m, 18H).

### Step 2

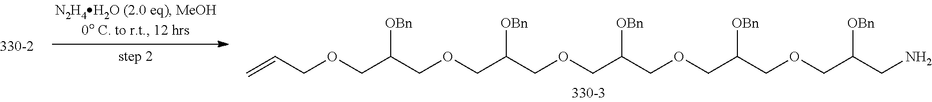 [SMILES: C=CCOCC(COCC(COCC(COCC(COCC(CN)OCc1ccccc1)OCc1ccccc1)OCc1ccccc1)OCc1ccccc1)OCc1ccccc1]

To a solution of 330-2 (1.472 g, 1.46 mmol) in MeOH (10 mL) was added N₂H₄·H₂O (146.3 mg, 2.92 mmol) in an ice bath, the resulting mixture was allowed to warm to r.t. and stirred for 10 hrs until 330-2 was consumed by TLC. The reaction mixture was concentrated to dryness under reduced pressure to give the crude product, which was dissolved in ethyl acetate (20 mL) and filtered. The filtrate was concentrated under reduced pressure to afford 330-3 (1.23 g, 1.402 mmol, 95.9%) as a colorless oil, which was used in the next step without further purification.

### Step 3

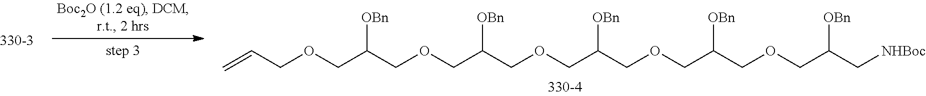 [SMILES: C=CCOCC(COCC(COCC(COCC(COCC(CNC(=O)OC(C)(C)C)OCc1ccccc1)OCc1ccccc1)OCc1ccccc1)OCc1ccccc1)OCc1ccccc1]

To a solution of 330-3 (1.23 g, 1.40 mmol) in DCM (8 mL) was added Boc₂O (367 mg. 1.68 mmol) at room temperature, and the reaction mixture was stirred at this temperature for 2 hrs until 330-3 was consumed and 330-4 was detected by LCMS. The reaction was concentrated under reduced pressure to give the crude product, which was purified with column chromatography (silica, 0-40% ethyl acetate in petroleum ether) affording 330-4 (1.23 g, 1.26 mmol, 89.8%) as a colorless oil. Purity=90%-95%.

### Step 4

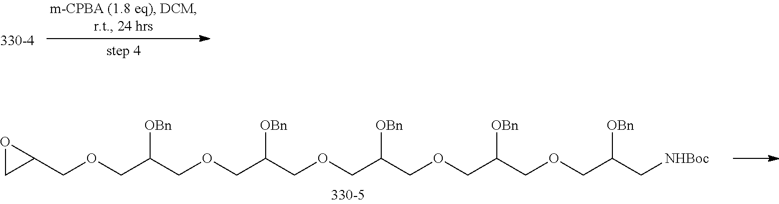 [SMILES: CC(C)(C)OC(=O)NCC(COCC(COCC(COCC(COCC(COCC1CO1)OCc1ccccc1)OCc1ccccc1)OCc1ccccc1)OCc1ccccc1)OCc1ccccc1]

To a solution of 330-4 (800 mg, 0.818 mmol) in DCM (5 mL) was added a solution of m-CPBA (254 mg, 1.473 mmol) in DCM (5 mL) at room-temperature, and the mixture was stirred for 24 hrs until 330-4 was consumed by TLC. The reaction was quenched by adding sat. Na₂S₂O₃ (5 mL) and sat. NaHCO₃ (5 mL), and the resulting mixture was stirred for 30 mins before diluting with DCM (60 mL). The organic phase was sequencely washed with sat. Na₂S₂O₃ (20 mL) and sat. NaHCO₃ (20 mL), dried over anhydrous Na₂SO₄, filtered and concentrated to dryness under reduced pressure. The crude product was purified with column chromatography (silica, 0-60% Ethyl acetate in petroleum ether) to afford 330-5 (689 mg, 0.693 mmol, 84.8%) as a colorless oil. Purity=90%-95%.

### Step 5

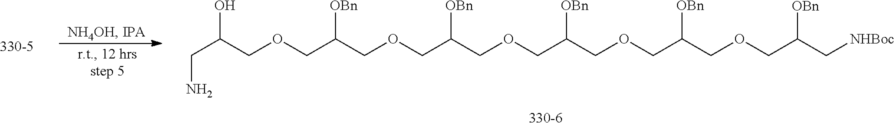 [SMILES: CC(C)(C)OC(=O)NCC(COCC(COCC(COCC(COCC(COCC(O)CN)OCc1ccccc1)OCc1ccccc1)OCc1ccccc1)OCc1ccccc1)OCc1ccccc1]

To a solution of 330-5 (689 mg, 0.693 mmol) in isopropyl alcohol (56 mL) was added ammonia (43 mL) dropwise at room temperature, and the resulting mixture was stirred at this temperature for 12 hours until 330-5 was consumed. The reaction mixture was concentrated to dryness under reduced pressure to afford 330-6 (698.7 mg, 0.691 mmol, 99.7%) as a yellow oil, which was used in the next step without further purification. Purity=90%-95%.

### Step 6

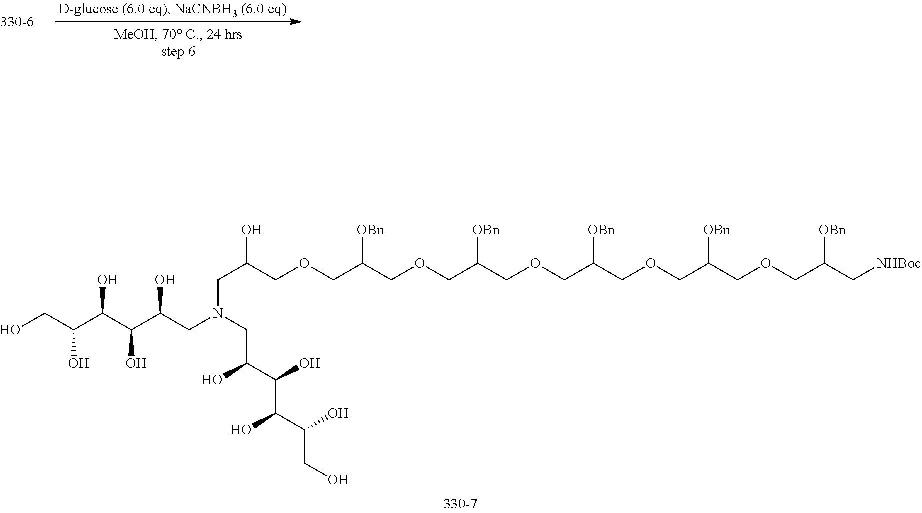 [SMILES: CC(C)(C)OC(=O)NCC(COCC(COCC(COCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1)OCc1ccccc1)OCc1ccccc1)OCc1ccccc1]

A mixture of 330-6 (983 mg, 0.973 mmol), D-glucose (1.05 g, 5.836 mmol) and NaCNBH₃ (366.7 mg, 5.836 mmol) in anhydrous MeOH (15 mL) was stirred at 70° C. for 24 hrs until most of 330-6 was consumed and 330-7 was detected by LCMS. The reaction mixture was cooled down to room temperature, filtered and concentrated under reduced pressure to give the crude product, which was purified by reverse phase liquid chromatography to give 330-7 (889 mg, 0.664 mmol, 68.2%) as a colorless oil. Purity=90%-95%.

### Step 7

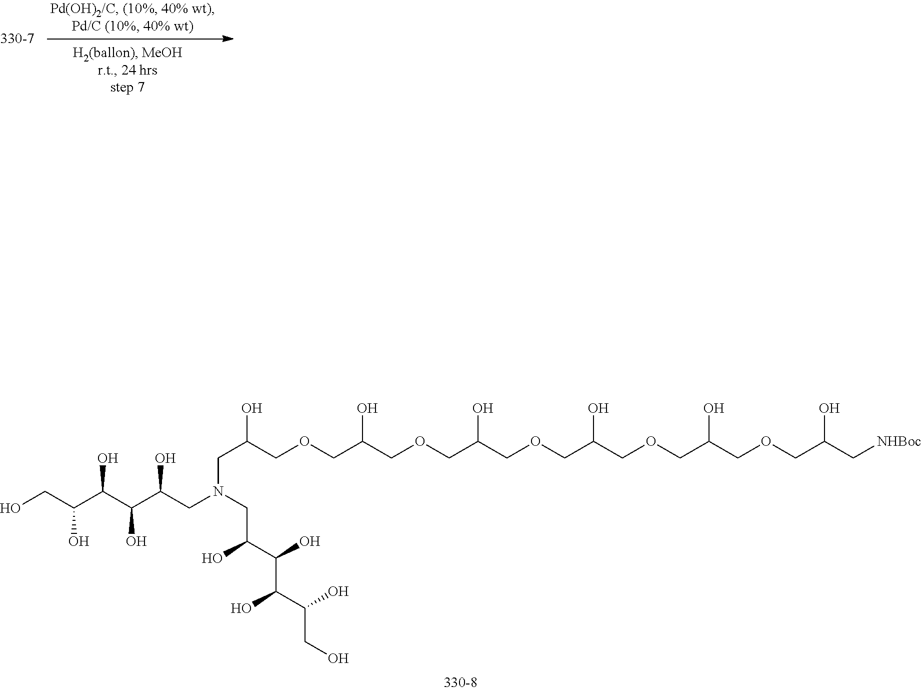 [SMILES: CC(C)(C)OC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

A mixture of 330-7 (270 mg, 0.202 mmol), Pd(OH)₂/C (10%, 120 mg) and Pd/C (10%, 120 mg) in MeOH (25 mL) was stirred under hydrogen atmosphere (balloon) for 24 h at room temperature until 330-7 was completely converted into 330-8. The reaction mixture was filtered through a celite pad, and the filtrate was concentrated to dryness under reduced pressure to afford 330-8 (138.2 mg, 0.156 mmol, 77.0%) as an off-white solid, which was used in the next step without further purification.

### Step 8

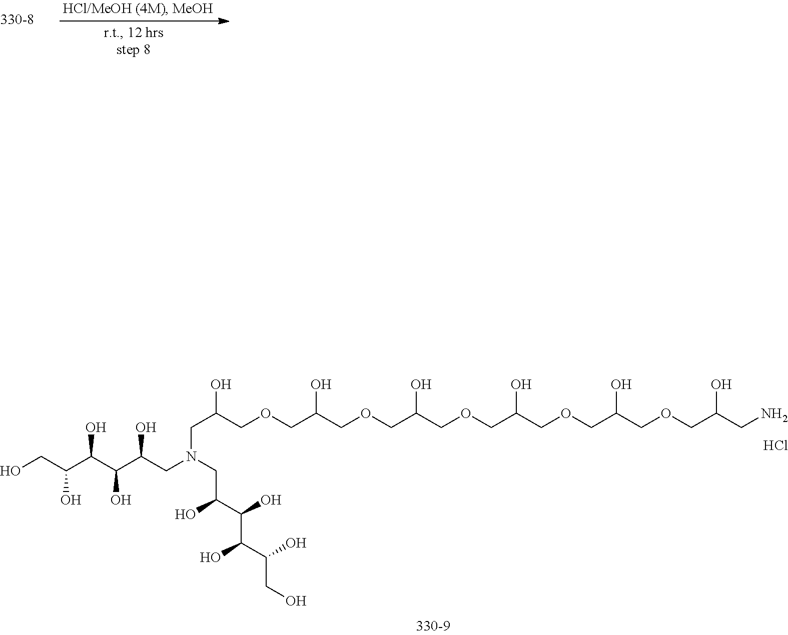 [SMILES: NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A solution of 330-8 (138 mg, 0.155 mmol) in HCl/MeOH (4M, 3 mL) and MeOH (3 mL) was stirred at room temperature for 12 hours until 330-8 was completely converted into 330-9. The reaction mixture was concentrated to dryness under reduced pressure to afford 330-9 (128 mg, 0.155 mmol, 100%) as an off-white solid, which was used in the next step without further purification.

### Step 9

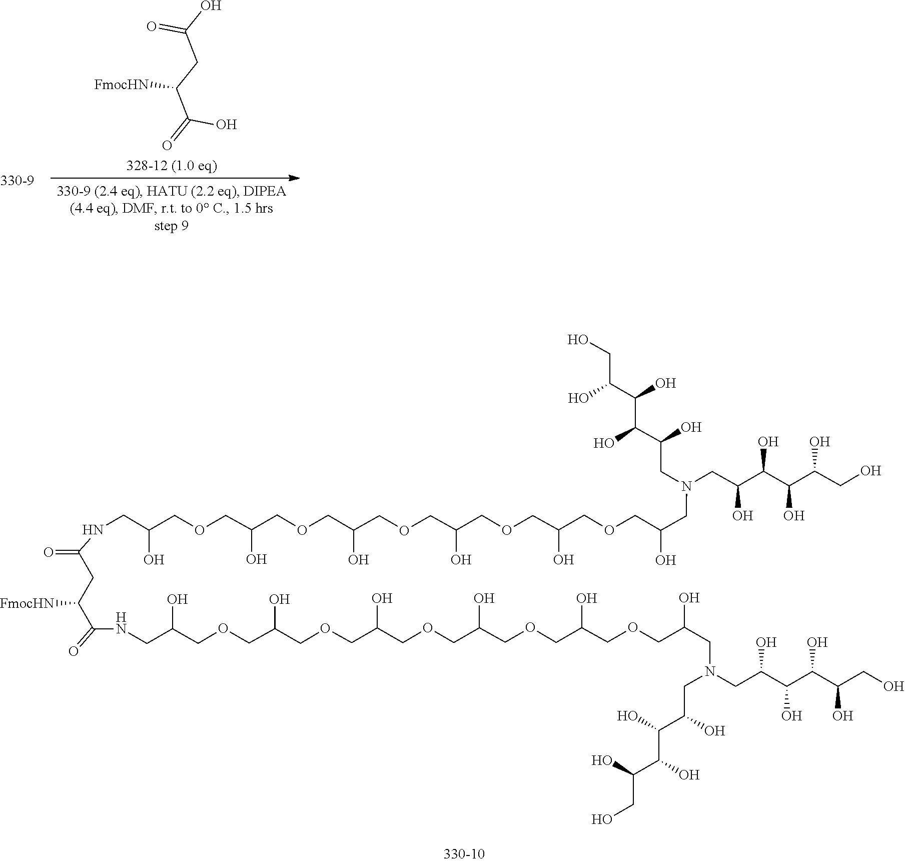

A solution of 328-12 (16.5 mg, 0.0465 mmol) and HATU (38.9 mg, 0.102 mmol) in anhydrous DMF (2 mL) was stirred at room temperature for 15 mins, then it was stirred in an ice bath. A solution of 330-9 (92 mg, 0.112 mmol) in anhydrous DMF (2 mL) was added dropwise, followed by DIPEA (26.5 mg, 0.205 mmol). The resulting mixture was stirred in the ice bath for 1 h until most of 328-12 was consumed. The reaction mixture was purified by reverse phase liquid chromatography to give 330-10 (14 mg, 0.00738 mmol, 15.9%) as a colorless oil.

### Step 10

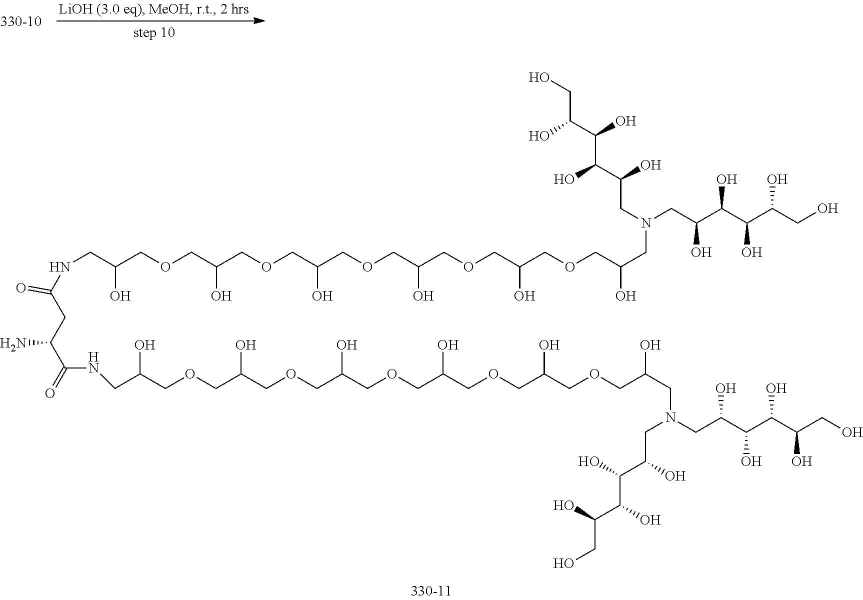 [SMILES: N[C@H](CC(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCC(O)COCC(O)COCC(O)COCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 330-10 (14 mg, 0.00738 mmol) in MeOH (2 mL) was added LiOH·H₂O (2 mg, 0.0442 mmol), and the mixture was stirred at room temperature for 2 hrs until 330-10 was consumed. The reaction solution was neutralized with 1N HCl to pH=7, and concentrated under reduced pressure to give a crude product, which was dissolved in H₂O (5 mL) and washed with hexane (2 mL×3). The aqueous phase was concentrated to dryness under reduced pressure to afford 330-11 (12.4 mg, 0.0074 mmol, 100%) as a white solid, which was used in the next step without further purification.

### Step 11

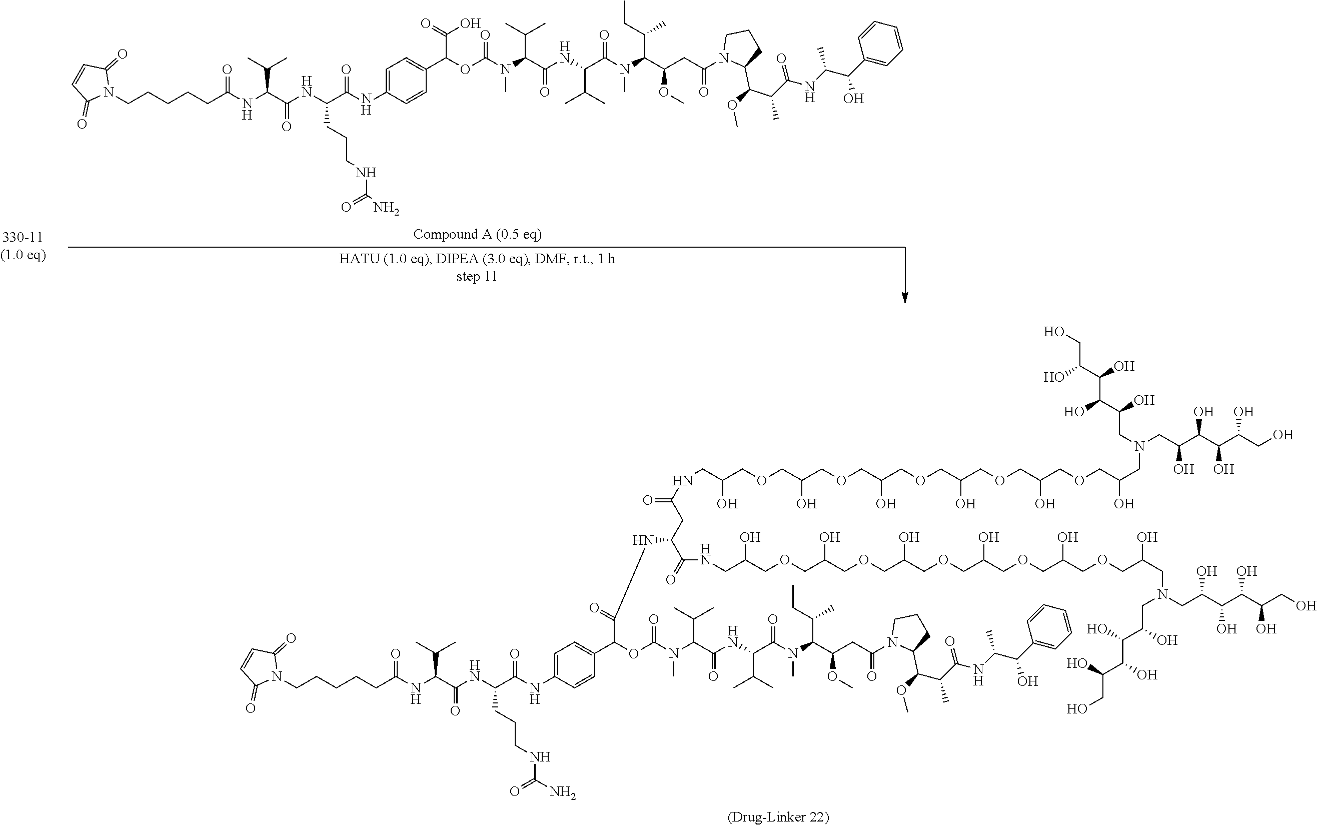

A solution of 330-11 (12 mg, 0.00716 mmol), Compound A (5.0 mg, 0.00368 mmol), HATU (2.7 mg, 0.0071 mmol) and DIPEA (2.8 mg, 0.0217 mmol) in anhydrous DMF (2 mL) was stirred at room temperature for 1 h until Compound A was consumed. Then the reaction solution was purified by prep-HPLC to give drug-linker 22 (5.0 mg, 0.00166 mmol, 45.1%) as a white solid. LCMS, m/z=1509.00 (M/2+H)⁺, m/z=1006.6 (M/3+H)⁺.

### Example 23: Preparation of Drug-Linker 23

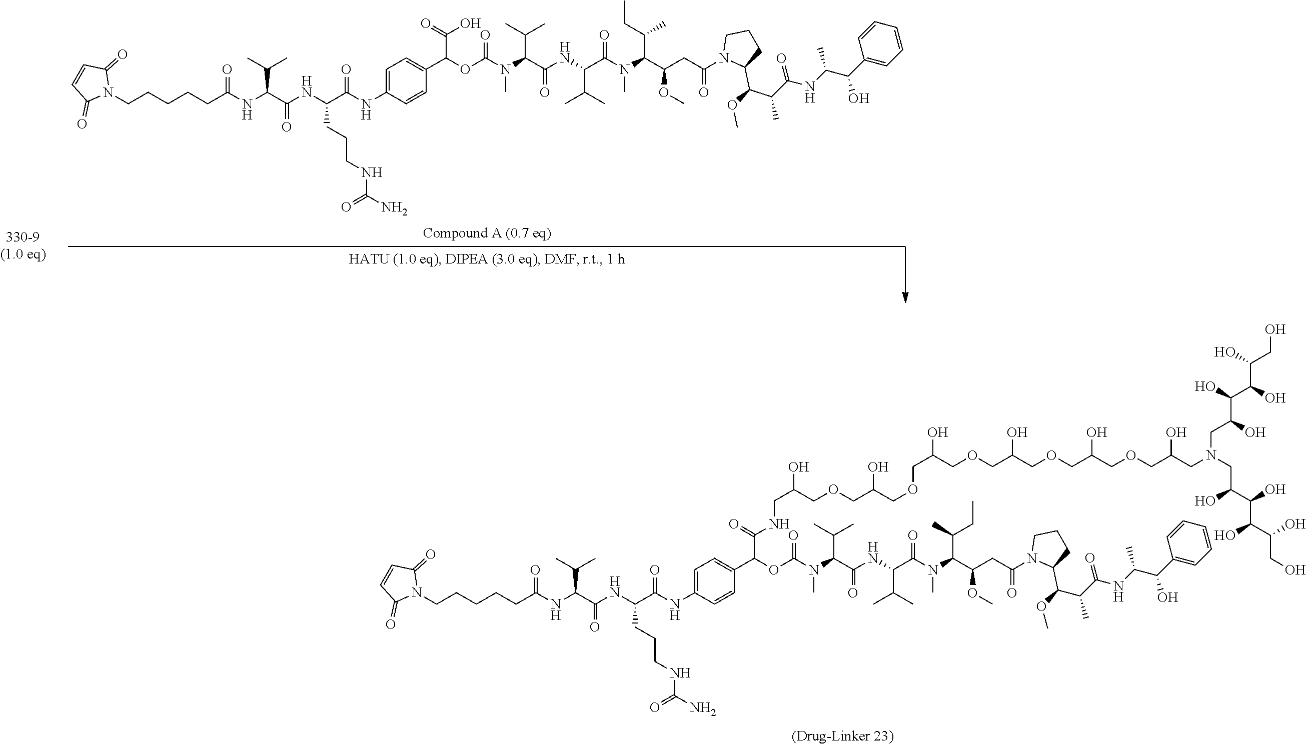

A solution of 330-9 (46 mg, 0.0558 mmol), Compound A (53 mg, 0.0390 mmol), HATU (21.2 mg, 0.0558 mmol) and DIPEA (21.6 mg, 0.167 mmol) in anhydrous DMF (4 mL) was stirred at room temperature for 1 h until Compound A was consumed. Then the reaction solution was purified by prep-HPLC to give drug-linker 23 (28 mg, 0.0131 mmol. 33.7%) as a white solid. LCMS, m/z=1066.11 (M/2+H)⁺. ¹H NMR (400 MHz, D₂O) δ 7.50-7.42 (m, 4H), 7.40-7.26 (m, 4H), 7.25-7.13 (m, 1H), 6.77 (s, 2H), 6.04-5.87 (m, 1H), 4.72-4.55 (m, 2H), 4.52-4.40 (m, 2H), 4.37-4.17 (m, 5H), 4.13-3.96 (m, 5H), 3.94-3.85 (m, 2H), 3.84-3.72 (m, 6H), 3.69-3.21 (m, 43H), 3.18-2.90 (m, 8H), 2.71 (s, 1H), 2.61-2.39 (m, 2H), 2.35-2.11 (m, 4H), 2.04 (d, J=9.8 Hz, 2H), 1.93-1.71 (m, 5H), 1.67-1.44 (m, 8H), 1.34-1.26 (m, 3H), 1.25-1.11 (m, 5H), 1.06 (d, J=6.4 Hz, 2H), 0.99-0.72 (m, 22H), 0.62-0.52 (m, 1H), 0.47-0.33 (m, 1H).

### Example 24: Preparation of Drug-Linker 24

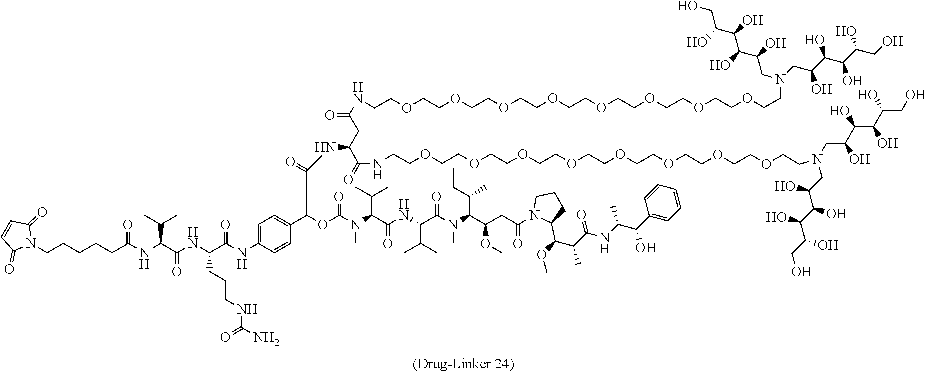 [SMILES: CC[C@H](C)[C@@H]([C@@H](CC(=O)N1CCC[C@H]1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OC(C(=O)N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)c1ccc(NC(=O)[C@H](CCCNC(N)=O)NC(=O)[C@@H](NC(=O)CCCCCN2C(=O)C=CC2=O)C(C)C)cc1)C(C)C]

### Step 1

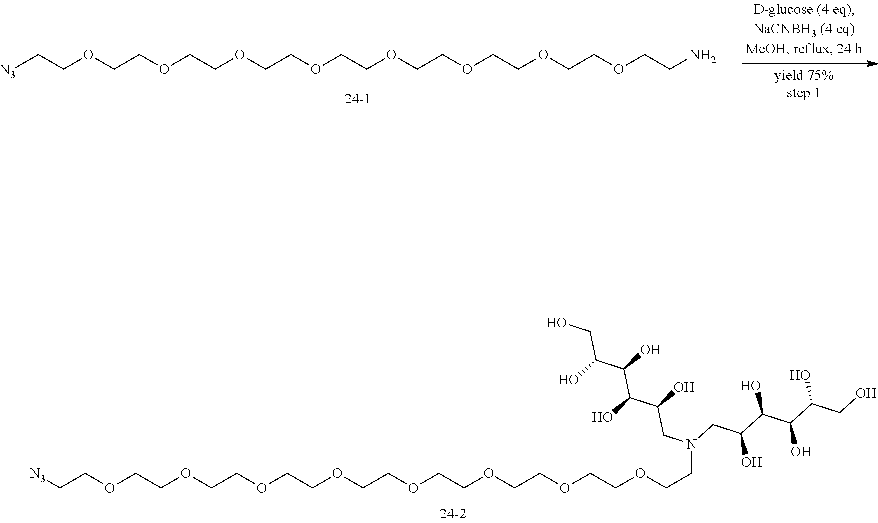 [SMILES: [N-]=[N+]=NCCOCCOCCOCCOCCOCCOCCOCCOCCN ; [N-]=[N+]=NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-1 (2.5 g, 5.701 mmol) in MeOH (20 mL) was added D-Glucose (4.11 g, 22.804 mmol) and NaBH₃CN (1.385 mL, 22.804 mmol). The mixture was stirred at reflux for 24h to complete. Then the resulting solution concentrated to dryness and the residue was purified by reverse phase chromatography (C8 column, eluting with 0-45% methanol in water with 0.01% TEA) to afford the product 24-2 as yellow oil. ESI m/z: 767.5 (M+H)⁺.

### Step 2

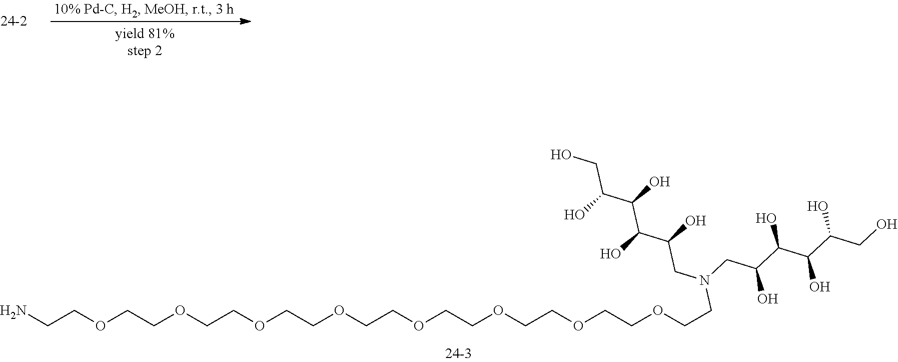 [SMILES: NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-2 (3.3 g, 4.303 mmol) in MeOH (20 mL) was added Pd/C (10% wt, 330 mg) under nitrogen and equipped with H₂ balloon. The reaction system was degassed and backfilled with hydrogen for three times and then stirred at room temperature under hydrogen atmosphere for 3h to complete. The resulting mixture was filtered to remove catalyst solid and the filtrate was concentrated, then purified by reverse phase chromatography (C8 column, eluting with 0-25% acetonitrile in water with 0.01% TFA) to afford the product 24-3 (2.6 g, 3.510 mmol, 81.50%) as colorless oil. ESI m/z: 371.3 (M/2+H)⁺, 741.4 (M+H)⁺.

### Step 3

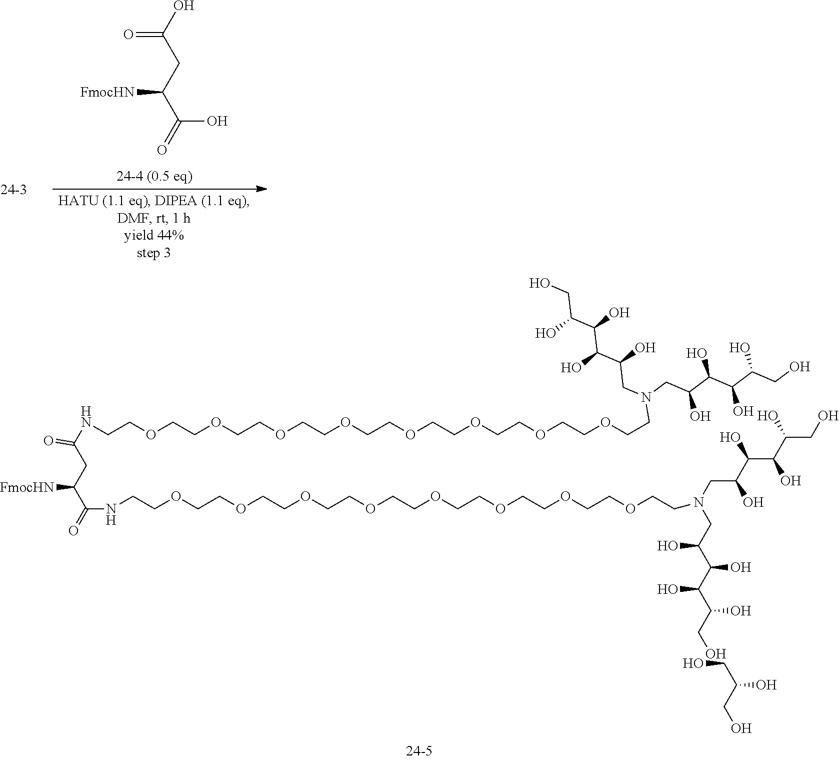

A solution of 24-4 (0.62 g, 1.755 mmol) in DMF (5 mL) was added HATU (1.47 g, 3.860 mmol) followed by DIPEA (0.50 g, 3.860 mmol). After stirring at room temperature for 15 min, the solution was added in dropwise manner into the solution of 24-3 (2.6 g, 3.510 mmol) in DMF (5 mL). After addition, the solution was stirred at room temperature for another 1 h to complete. The completed solution was then purified directly by reverse phase chromatography (C8 column, eluting with 0-40% acetonitrile in water with 0.01% TFA) to afford the product 24-5 (1.4 g, 0.777 mmol, 44.30%) as colorless oil. ESI m/z: 601.0 (M/3+H)⁺, 901.0 (M/2+H)⁺.

### Step 4

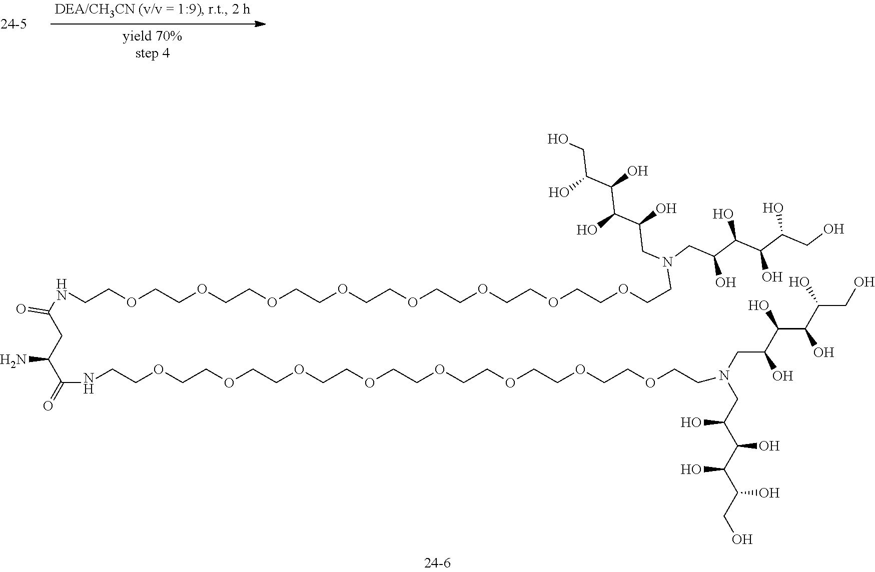 [SMILES: N[C@@H](CC(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCOCCOCCN(C[C@H](O)[C@@H](O)[C@H](O)[C@H](O)CO)C[C@H](O)[C@@H](O)[C@H](O)[C@H](O)CO]

To the solution of 24-5 (1.4 g, 0.777 mmol) in MeCN (6 mL) was diethyl amine (0.7 mL, 8.930 mmol). The mixture was stirred at room temperature for 2h to achieve complete deprotection. Then the resulting solution was concentrated under reduced pressure to remove most of diethyl amine, and the residue was purified by reverse phase chromatography (C8 column, eluting with 0-20% acetonitrile in water with 0.01% TFA) to get desired fractions, which was freeze-dried to afford the product 24-6 (0.86 g, 0.545 mmol, 69.92%) as sticky colorless oil. ESI m/z: 526.9 (M/3+H)⁺, 789.9 (M/2+H)⁺. 1HNMR (400 MHz, DMSO-d6) δ 8.36 (t, J=5.6 Hz, 1H), 8.24 (t, J=5.6 Hz, 1H), 5.88-4.41 (m, 15H), 3.99-3.79 (m, 4H), 3.61-3.56 (m, 12H), 3.52-3.49 (m, 60H), 3.49-3.40 (m, 12H), 3.27-3.19 (m, 6H), 3.04-2.86 (m, 16H), 2.65-2.06 (m, 2H), 1.15 (t, J=7.2 Hz, 2H) ppm.

### Step 5

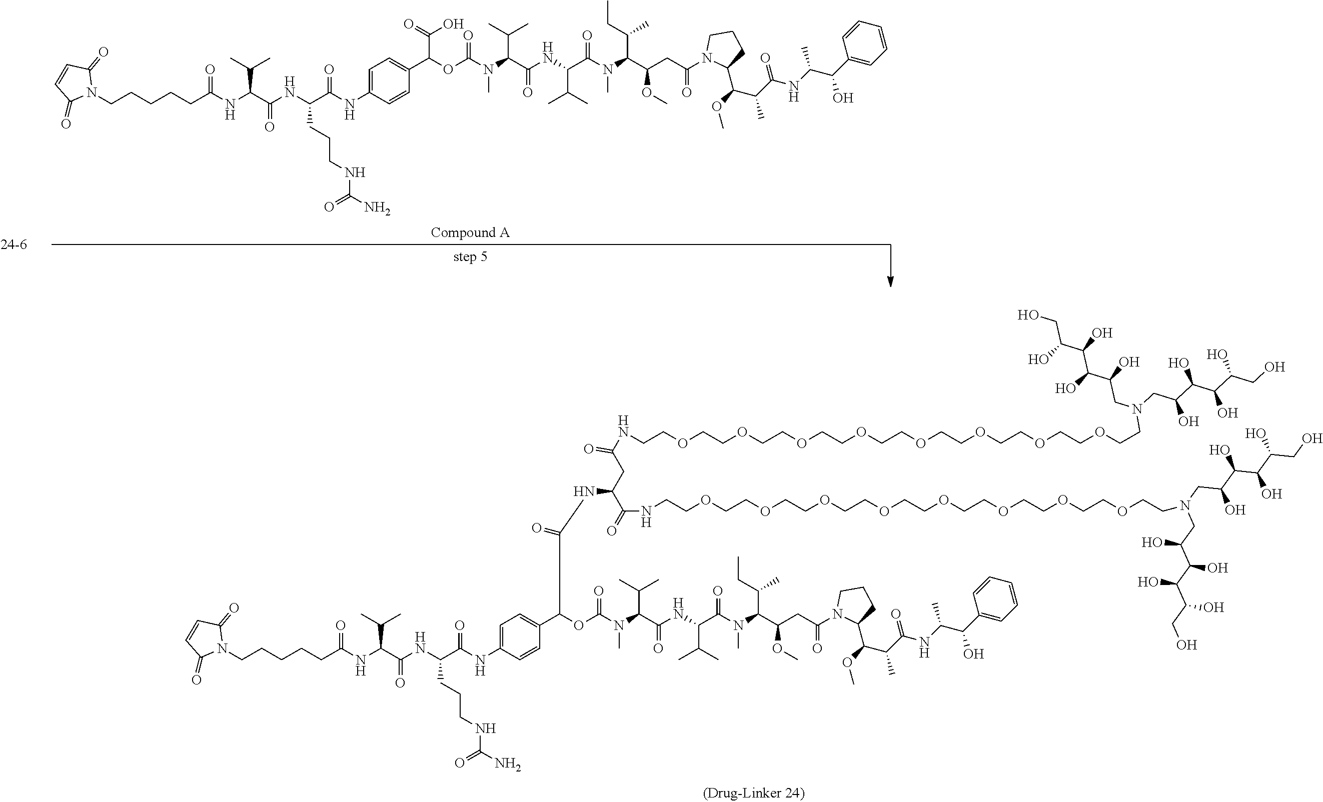

A solution of 24-6 (100 mg, 0.063 mmol), Compound A (86 mg, 0.063 mmol) and HATU (24 mg, 0.063 mmol) in anhydrous DMF (4 mL) was stirred at room temperature for 5 min, then DIPEA (25 mg, 0.193 mmol) was added. The resulting solution was stirred for another 1 hr at r.t. until LCMS indicated complete reaction. Then the reaction solution was purified by prep. HPLC to give drug-linker 24 (40 mg, 0.014 mmol, 21.73%) as a white solid. LCMS, m/z=1461.23 (M/2+H)⁺, ¹H NMR (400 MHz, D₂O) δ 7.54-7.48 (m, 2H), 7.45-7.37 (m, 2H), 7.37-7.26 (m, 5H), 6.73 (s, 2H), 4.40-4.38 (m, 2H), 4.19-4.15 (m, 6H), 3.85-3.83 (m, 5H), 3.78-3.74 (m, 1OH), 3.71-3.66 (m, 13H), 3.63-3.61 (m, 49H), 3.58-3.56 (m, 17H), 3.49-3.43 (m, 14H), 3.39-3.35 (m, 4H), 3.30-3.29 (d, 3H), 3.24-3.23 (d, 4H), 3.06-3.00 (m, 4H), 2.77-2.64 (m, 2H), 2.22-2.21 (m, 2H), 1.99-1.97 (m, 2H), 1.75 (s, 5H), 1.53-1.45 (m, 1OH), 1.24-1.08 (m, 9H), 1.02-1.01 (m, 2H), 0.92-0.75 (m, 28H) ppm.

### Example 25: Preparation on Drug-Linker 25

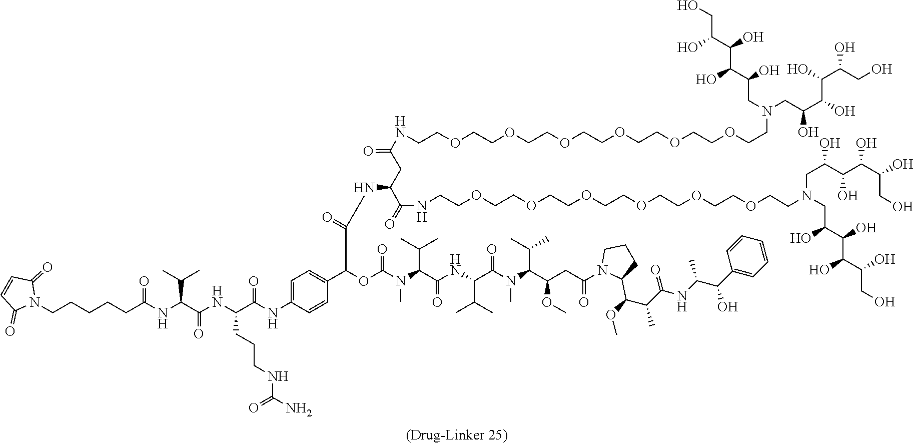

### Step 1

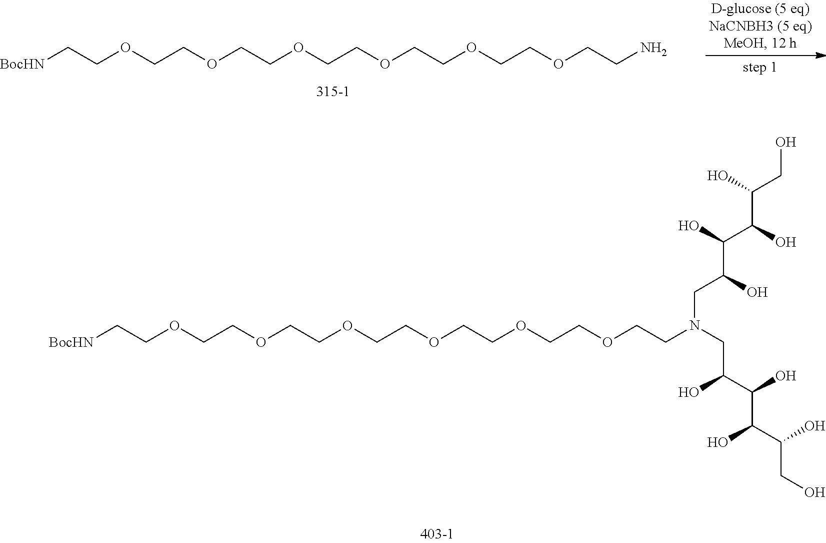 [SMILES: CC(C)(C)OC(=O)NCCOCCOCCOCCOCCOCCOCCN ; CC(C)(C)OC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 315-1 (1.0 g, 2.355 mmol) and D-Glucose (2.1 g, 11.656 mmol) in anhydrous Methanol (40 mL) was heated at 50° C. for 30 min, then NaCNBH₃ (740 mg, 11.776 mmol) was added. The resulting solution was stirred for another 12 hr at 70° C. until indicated all starting amine was disappeared and the mass of desired product was detected. Then the reaction solution was concentrated and purified by reverse phase liquid chromatography to give 403-1 (730 mg, 0.970 mmol, 41.17%) as a white solid.

### Step 2

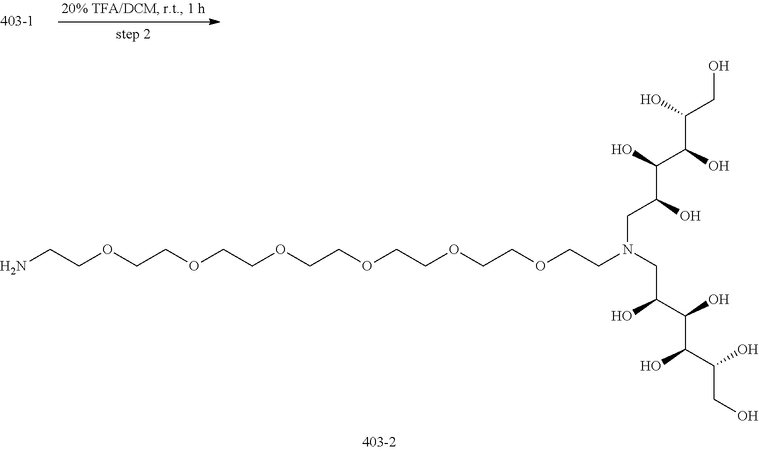 [SMILES: NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-1 (730 mg, 0.970 mmol) and TFA (2 mL) in anhydrous DCM (8 mL) was stirred at room temperature for 1 hr until LCMS indicated all starting amine was disappeared and desired product was detected. Then the solution was concentrated to dryness to 403-2 (610 mg, 0.935 mmol, 96.39%) as yellow oil, used as such in the next step.

### Step 3

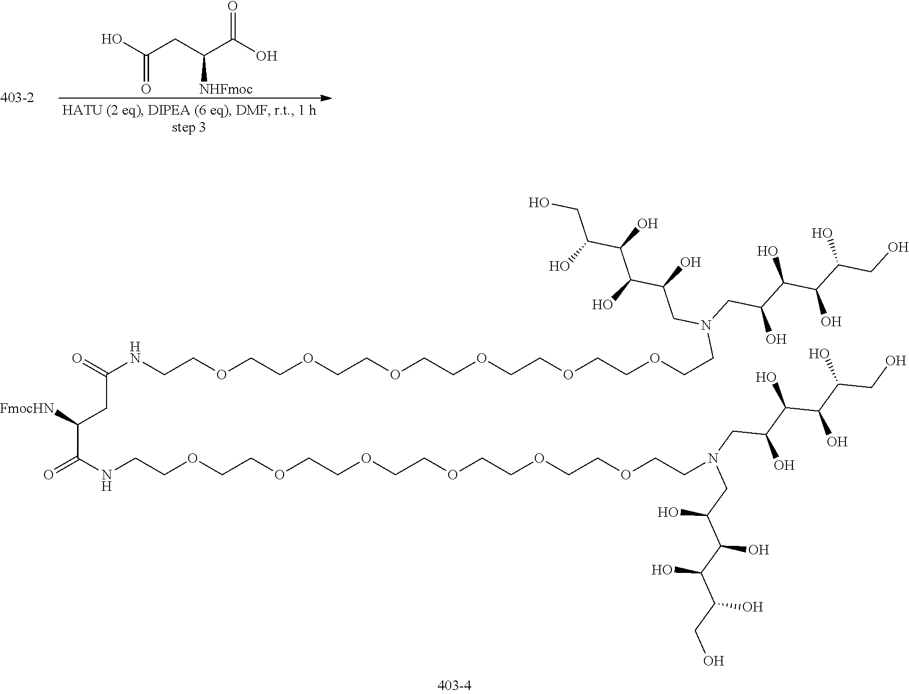

A solution of 403-2 (610 mg, 0.935 mmol), 403-3 (166 mg, 0.467 mmol) and HATU (355 mg, 0.934 mmol) in anhydrous DMF (5 mL) was stirred at room temperature for 5 min, then DIPEA (362 mg, 2.801 mmol) was added. The resulting solution was stirred for another 1 hr at r.t. until LCMS indicated complete reaction. The reaction solution was purified directly by reverse phase liquid chromatography to give 403-4 (410 mg, 0.252 mmol, 54.03%) as a white solid.

### Step 4

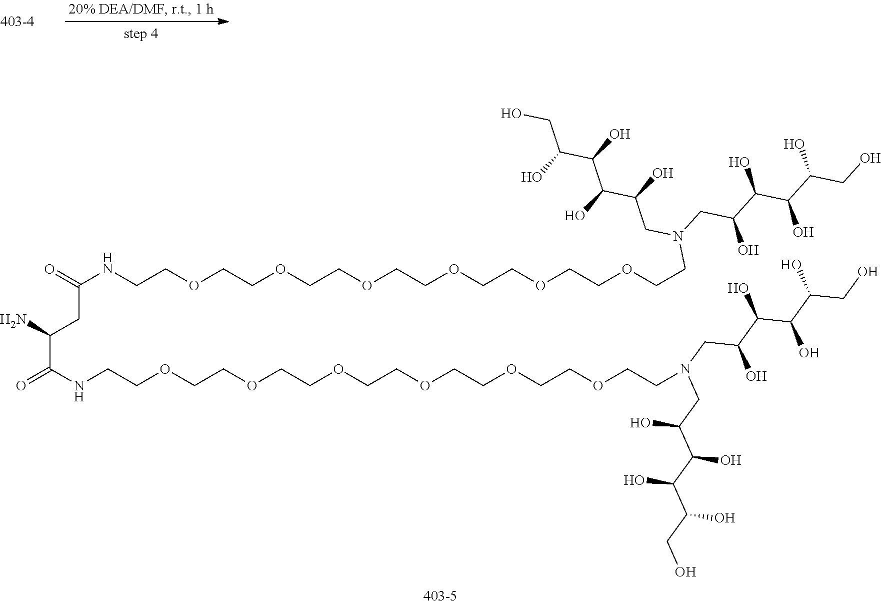 [SMILES: N[C@@H](CC(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO)C(=O)NCCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 403-4 (200 mg, 0.123 mmol) and DEA (1 mL) in anhydrous DMF (4 mL) was stirred at room temperature for 1 hr until LCMS indicated all starting amine was disappeared and desired product was detected. Then the solution was concentrated to dryness to 403-5 (167 mg, 0.119 mmol, 96.80%) as colorless oil, used as such in the next step.

### Step 5

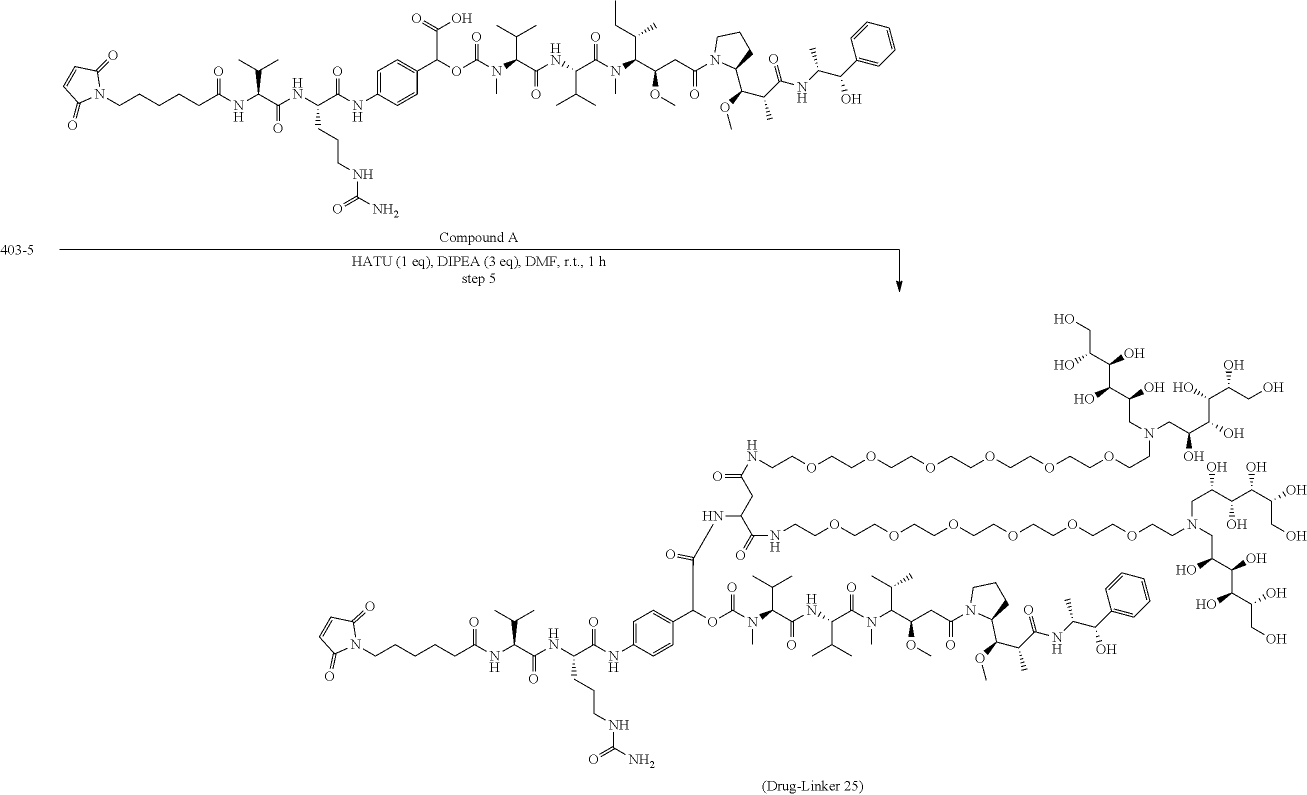

A solution of 403-5 (41 mg, 0.029 mmol), Compound A (40 mg, 0.029 mmol) and HATU (11 mg, 0.029 mmol) in anhydrous DMF (4 mL) was stirred at room temperature for 5 min, then DIPEA (11 mg, 0.085 mmol) was added. The resulting solution was stirred for another 1 hr at r.t. until LCMS indicated complete reaction. Then the reaction solution was purified by prep. HPLC to give drug-linker 25 (18 mg, 0.007 mmol, 22.61%) as a white solid. LCMS, m/z=1373.31 (M/2+H)⁺.

### Example 26: Preparation of Drug-Linker 26

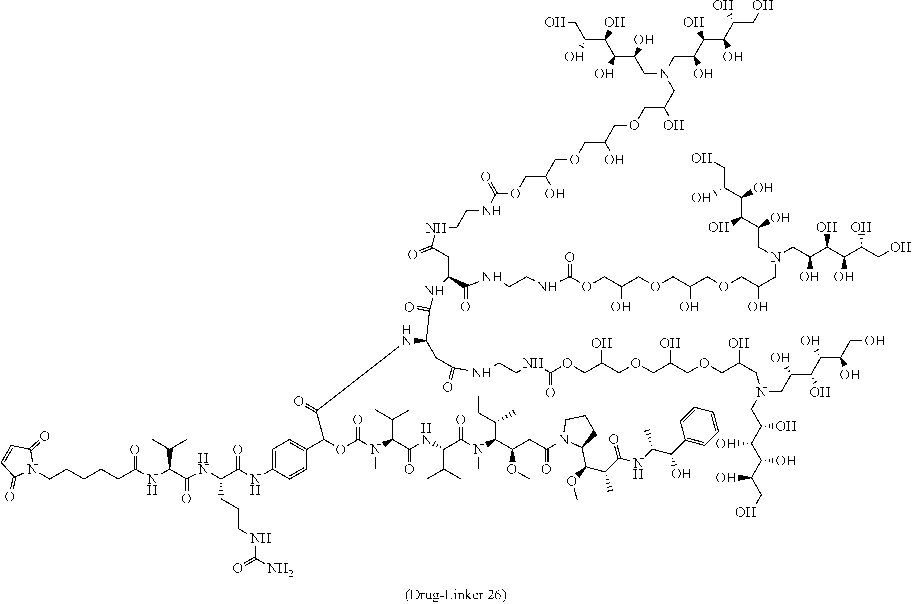

### Step 1

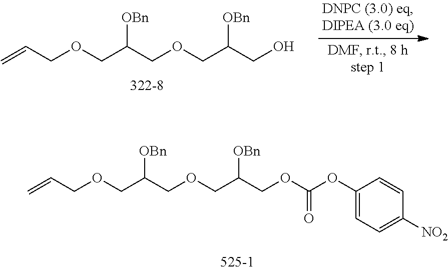 [SMILES: C=CCOCC(COCC(CO)OCc1ccccc1)OCc1ccccc1 ; C=CCOCC(COCC(COC(=O)Oc1ccc([N+](=O)[O-])cc1)OCc1ccccc1)OCc1ccccc1]

To a solution of 322-8 (600 mg, 1.554 mmol) in DMF (12 mL) was added DIPEA (602.4 mg, 4.661 mmol), followed by 4,4′-dinitrodiphenyl carbonate (1.42 g, 4.661 mmol), then the resulting mixture was stirred at room temperature for 8 hrs until 322-8 was consumed detected by LCMS. The reaction solution was directly used in the next step without work-up procedure.

### Step 2

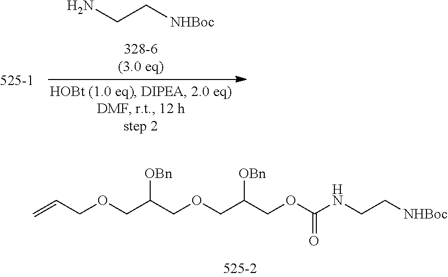 [SMILES: CC(C)(C)OC(=O)NCCN ; C=CCOCC(COCC(COC(=O)NCCNC(=O)OC(C)(C)C)OCc1ccccc1)OCc1ccccc1]

To the above reaction mixture was added HOBt (210 mg, 1.554 mmol), DIPEA (401.7 mg, 3.108 mmol) and 328-6 (746.5 mg, 4.662 mmol) successively, and the resulting mixture was stirred at room temperature for 6 hrs until 525-1 was consumed detected by LCMS. The reaction mixture was diluted with ethyl acetate (180 mL) and washed with saturated NaHCO₃ (aq, 45 mL×3), dried over anhydrous Na₂SO₄, filtered and concentrated to dryness under reduced pressure. The crude product was purified with column chromatography (silica, 0-80% ethyl acetate in petroleum ether) affording 525-2 (893 mg, 1.56 mmol, 100.4% over 2 steps) as a pale yellow oil.

### Step 3

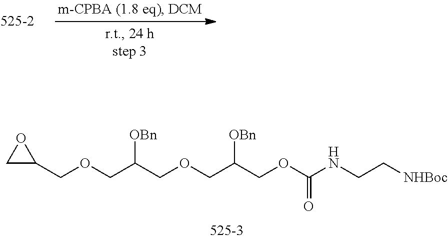 [SMILES: CC(C)(C)OC(=O)NCCNC(=O)OCC(COCC(COCC1CO1)OCc1ccccc1)OCc1ccccc1]

To a solution of 525-2 (890 mg, 1.555 mmol) in DCM (10 mL) was added a solution of m-CPBA (483 mg, 2.80 mmol) in DCM (10 mL) dropwise at room temperature. The resulting mixture was stirred at this temperature for 24 hours until 525-2 was consumed, and the reaction was quenched with saturated Na₂S₂O₃ (aq, 10 mL) and NaHCO₃ (aq., 10 mL). The reaction mixture was stirred for 30 mins, and then diluted with DCM (50 mL). The organic phase was washed with a mixture solution (20 mL×3) of saturated Na₂S₂O₃ and NaHCO₃ (aq, 1:1, V/V), dried over anhydrous Na₂SO₄, filtered and concentrated to dryness under reduced pressure to give the crude product, which was purified with column chromatography (silica, 0-100% ethyl acetate in petroleum ether) affording 525-3 (790 mg, 1.343 mmol, 86.4%) as a pale yellow oil.

### Purity=90%-95%.

### Step 4

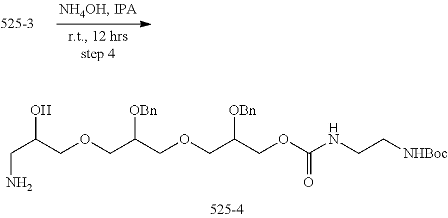 [SMILES: CC(C)(C)OC(=O)NCCNC(=O)OCC(COCC(COCC(O)CN)OCc1ccccc1)OCc1ccccc1]

To a solution of 525-3 (790 mg, 1.343 mmol) in isopropyl alcohol (110 mL) was added ammonia (110 mL) dropwise at room temperature, and the resulting mixture was stirred at this temperature for 12 hours until 525-3 was consumed. The reaction mixture was concentrated to dryness under reduced pressure to afford 525-4 (801.2 mg, 1.323 mmol, 98.6%) as a yellow oil, which was used in the next step without further purification. Purity=90%-95%.

### Step 5

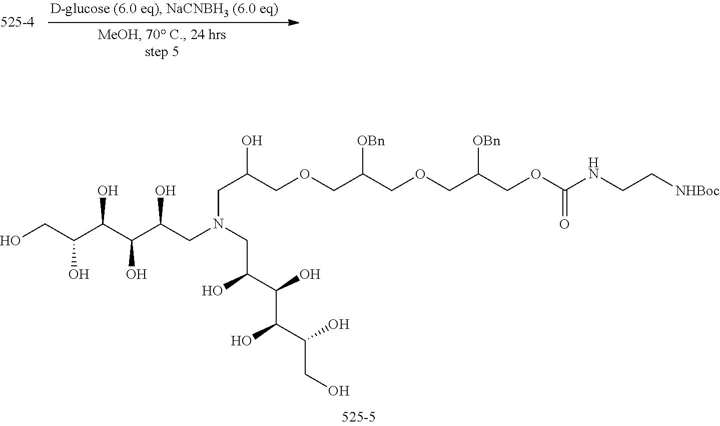 [SMILES: CC(C)(C)OC(=O)NCCNC(=O)OCC(COCC(COCC(O)CN(C[C@H](O)[C@@H](O)[C@H](O)[C@H](O)CO)C[C@H](O)[C@@H](O)[C@H](O)[C@H](O)CO)OCc1ccccc1)OCc1ccccc1]

A mixture of 525-4 (801.2 mg, 1.324 mmol), D-glucose (1.43 g, 7.937 mmol) and NaCNBH₃ (499.2 mg, 7.94 mmol) in anhydrous MeOH (21 mL) was stirred at 70° C. for 24 hrs until most of 525-4 was consumed and 525-5 was detected by LCMS. The reaction mixture was cooled down to room temperature, filtered and concentrated under reduced pressure to give the crude product, which was purified by reverse phase liquid chromatography to give 525-5 (1.2 g, 1.29 mmol, 97.1%) as a colorless oil. Purity=85%-90%.

### Step 6

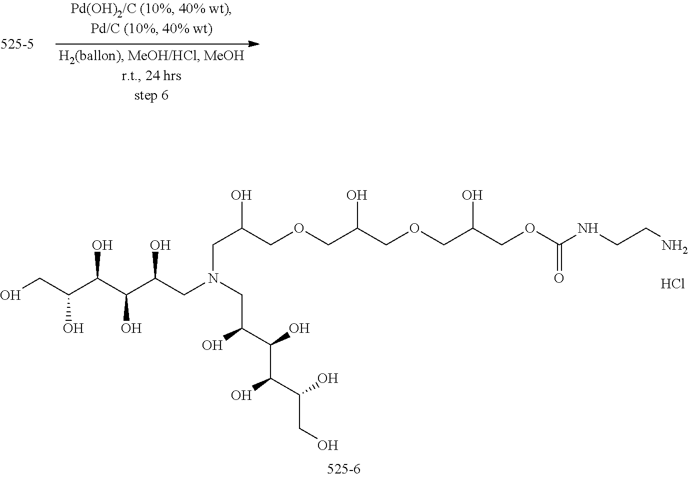 [SMILES: NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO ; Cl]

A mixture of 525-5 (1.2 g, 1.286 mmol), Pd(OH)₂/C (10%, 250 mg) and Pd/C (10%, 250 mg) in HCl/MeOH (4M, 25 mL), MeOH (25 mL) was stirred under hydrogen atmosphere (ballon) for 24 h at room temperature until 525-5 was completely converted into 525-6. The reaction mixture was filtered through a celite pad, and the filtrate was concentrated to dryness under reduced pressure to afford 525-6 (886 mg, 1.285 mmol, 100.0%) as an off-white solid, which was used in the next step without further purification.

### Step 7

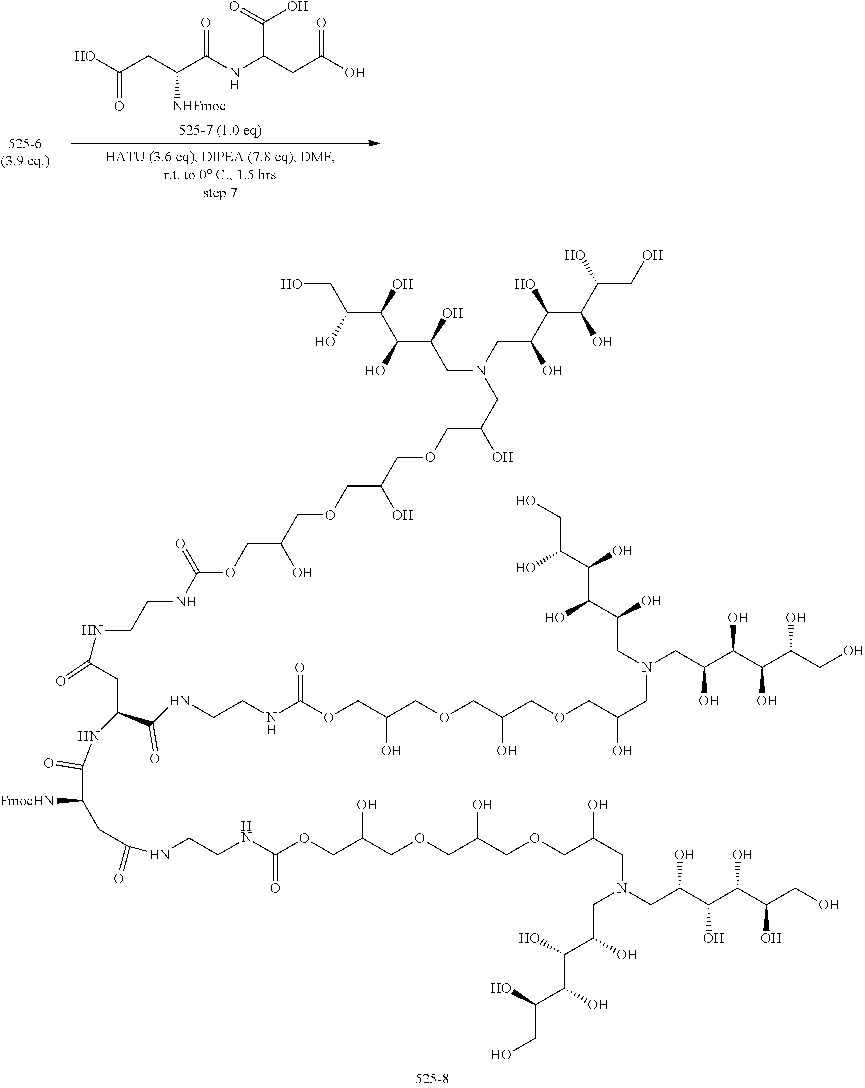

A solution of 525-7 (104.9 mg, 0.223 mmol) and HATU (305.4 mg, 0.803 mmol) in anhydrous DMF (3 mL) was stirred at room temperature for 15 mins, then it was stirred in an ice bath. A solution of 525-6 (600 mg, 0.870 mmol) in anhydrous DMF (3 mL) was added dropwise, followed by DIPEA (225 mg, 1.74 mmol). The resulting mixture was stirred in the ice bath for 1 h until most of 526-7 was consumed. The reaction mixture was purified by reverse phase liquid chromatography to give 525-8 (269.8 mg, 0.113 mmol, 50.9%) as a white solid. Purity=90%-95%. ¹H NMR (400 MHz, DMSO-d₆) δ 7.87 (d, J=7.6 Hz, 2H), 7.64 (d, J=7.2 Hz, 2H), 7.47 (t, J=7.6 Hz, 2H), 7.42-7.32 (m, 2H), 4.65-4.57 (m, 1H), 4.53-4.36 (m, 3H), 4.33-4.13 (m, 1 OH), 4.08-3.87 (m, 12H), 3.84-3.69 (m, 18H), 3.67-3.35 (m, 53H), 3.30-3.07 (m, 12H), 2.77-2.32 (m, 4H).

### Step 8

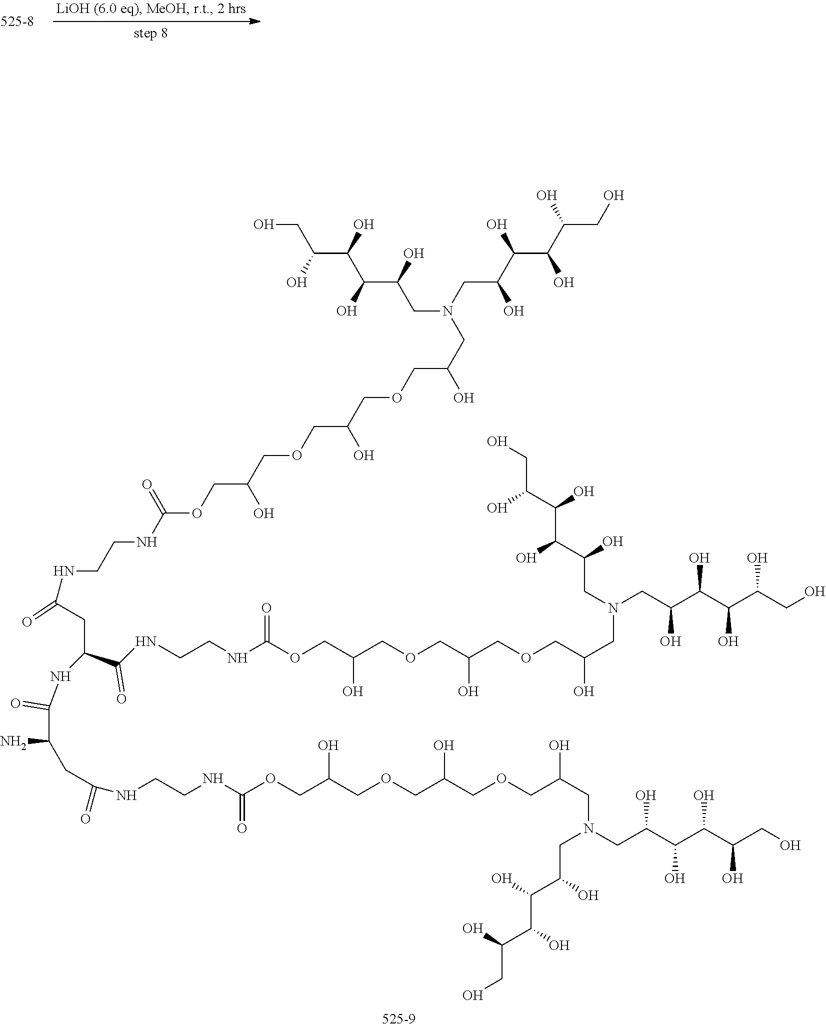 [SMILES: N[C@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)N[C@@H](CC(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO)C(=O)NCCNC(=O)OCC(O)COCC(O)COCC(O)CN(C[C@H](O)[C@@H](O)[C@H](O)[C@H](O)CO)C[C@H](O)[C@@H](O)[C@H](O)[C@H](O)CO]

To a solution of 525-8 (100 mg, 0.0421 mmol) in MeOH (3 mL) and H₂O (1 mL) was added LiOH·H₂O (10.6 mg, 0.252 mmol), and the mixture was stirred at room temperature for 2 hrs until 525-8 was consumed. The reaction solution was neutralized with 1N HCl to pH=7, and concentrated under reduced pressure to give a crude product, which was dissolved in H₂O (15 mL) and washed with hexane (10 mL×3). The aqueous phase was concentrated to dryness under reduced pressure to afford 525-9 (74.6 mg, 0.0346 mmol, 82.3%) as a colorless oil, which was used in the next step without further purification.

### Step 9

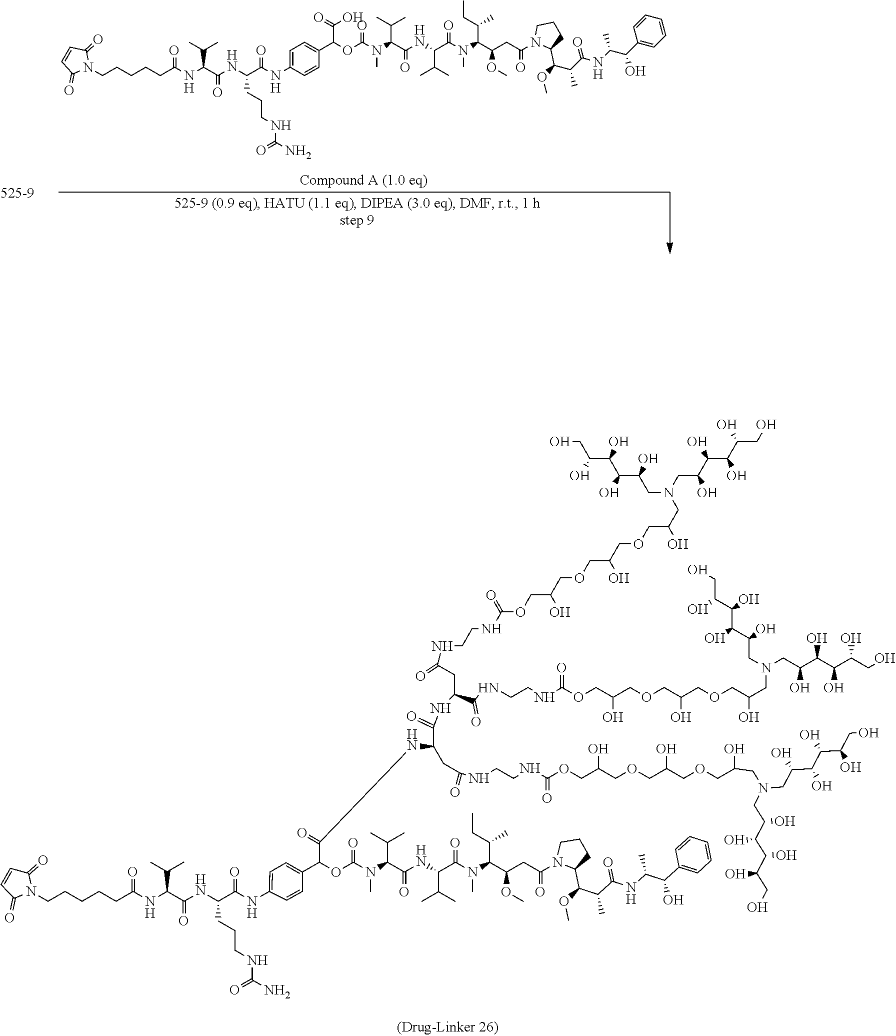

A solution of 525-9 (70.0 mg, 0.0325 mmol), Compound A (49 mg, 0.0360 mmol), HATU (15.1 mg, 0.0397 mmol) and DIPEA (14.0 mg, 0.108 mmol) in anhydrous DMF (4 mL) was stirred at room temperature for 1 h until Compound A was consumed. Then the reaction solution was purified by prep-HPLC to give drug-linker 26 (23.5 mg, 0.00672 mmol, 20.7%) as a white solid. LCMS, m/z=1749.66 (M/2+H)⁺, m/z=1166.72 (M/2+H)⁺. ¹H NMR (400 MHz, DMSO-d₆) δ 7.61-7.40 (m, 4H), 7.39-7.26 (m, 4H), 7.25-7.13 (m, 1H), 6.77 (s, 2H), 5.98 (t, J=13.6 Hz, 1H), 4.75-4.52 (m, 7H), 4.49-4.38 (m, 2H), 4.37-3.90 (m, 25H), 3.87-3.38 (m, 73H), 3.37-2.87 (m, 28H), 2.87-2.08 (m, 12H), 2.07-1.95 (m, 2H), 1.93-1.65 (m, 5H), 1.68-1.41 (m, 8H), 1.37-1.11 (m, 8H), 1.09-1.02 (m, 2H), 1.00-0.75 (m, 20H), 0.74-0.67 (m, 1H), 0.57-0.48 (m, 1H), 0.33 (d, J=6.4 Hz, 1H).

### Example 27: Preparation of Drug-Linker 27

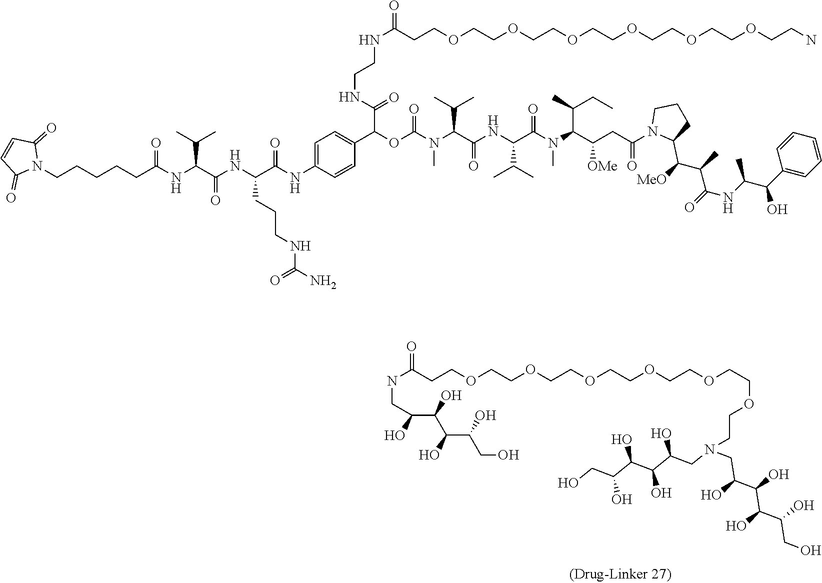 [SMILES: CC[C@H](C)[C@@H]([C@H](CC(=O)N1CCC[C@H]1[C@H](OC)[C@@H](C)C(=O)N[C@@H](C)[C@H](O)c1ccccc1)OC)N(C)C(=O)[C@@H](NC(=O)[C@H](C(C)C)N(C)C(=O)OC(C(=O)NCCNC(=O)CCOCCOCCOCCOCCOCCOCCN)c1ccc(NC(=O)[C@H](CCCNC(N)=O)NC(=O)[C@@H](NC(=O)CCCCCN2C(=O)C=CC2=O)C(C)C)cc1)C(C)C ; O=C(CCOCCOCCOCCOCCOCCOCCN(C[C@H](O)[C@@H](O)[C@H](O)[C@H](O)CO)C[C@H](O)[C@@H](O)[C@H](O)[C@H](O)CO)NC[C@H](O)[C@@H](O)[C@H](O)[C@H](O)CO]

### Step 1

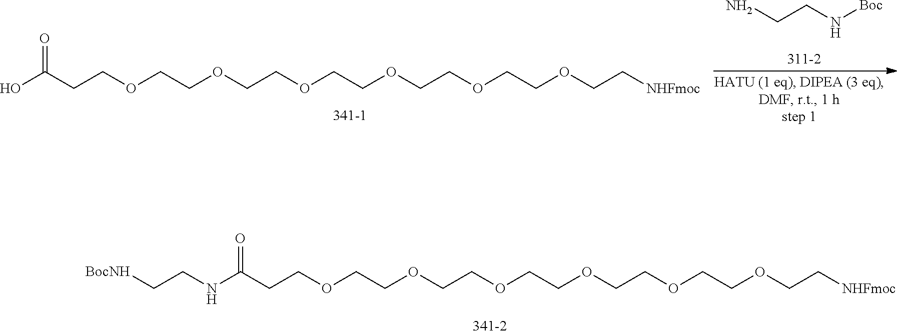

A solution of 341-1 (1 g, 1.737 mmol), 311-2 (278 mg, 1.735 mmol) and HATU (661 mg, 1.738 mmol) in anhydrous DMF (10 mL) was stirred at room temperature for 5 min, then DIPEA (674 mg, 5.215 mmol) was added. The resulting solution was stirred for another 1 hr at r.t. until LCMS indicated complete reaction. The reaction solution was purified directly by reverse phase liquid chromatography to give 341-2 (950 mg, 1.323 mmol, 76.17%) as a white solid. purity=90%-95%.

### Step 2

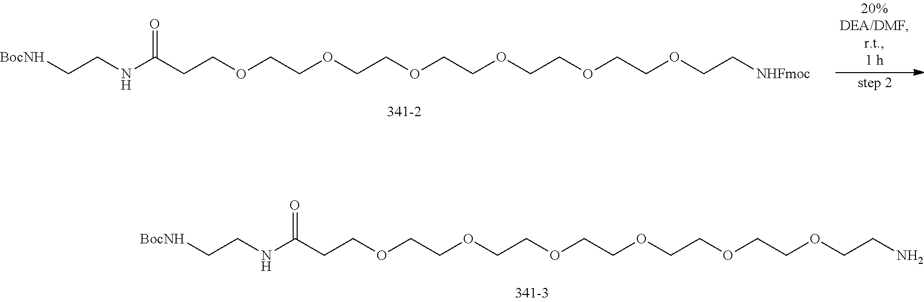

A solution of 341-2 (950 mg, 1.323 mmol) and DEA (2 mL) in anhydrous DMF (8 mL) was stirred at room temperature for 1 hr until LCMS indicated all starting amine was disappeared and desired product was detected. Then the solution was concentrated to dryness to 341-3 (610 mg, 1.231 mmol, 93.05%) as colorless oil, used as such in the next step. purity=90%-95%.

### Step 3

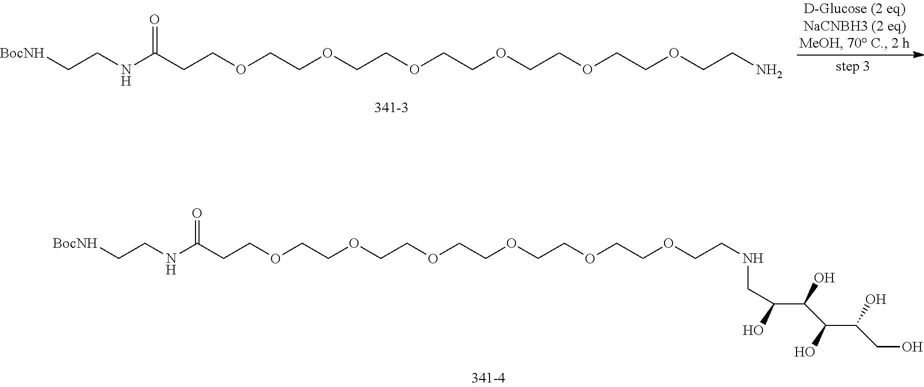 [SMILES: CC(C)(C)OC(=O)NCCNC(=O)CCOCCOCCOCCOCCOCCOCCN ; CC(C)(C)OC(=O)NCCNC(=O)CCOCCOCCOCCOCCOCCOCCNC[C@H](O)[C@@H](O)[C@H](O)[C@H](O)CO]

A solution of 341-3 (610 mg, 1.231 mmol) and D-Glucose (444 mg, 2.464 mmol) in anhydrous Methanol (50 mL) was heated at 50° C. for 30 min, then NaCNBH₃ (155 mg, 2.467 mmol) was added. The resulting solution was stirred for another 2 hr at 70° C. until indicated all starting amine was disappeared and the mass of desired product was detected. Then the reaction solution was concentrated and purified by reverse phase liquid chromatography to give 341-4 (340 mg, 0.515 mmol, 41.84%) as a white solid. purity=90%-95%.

### Step 4

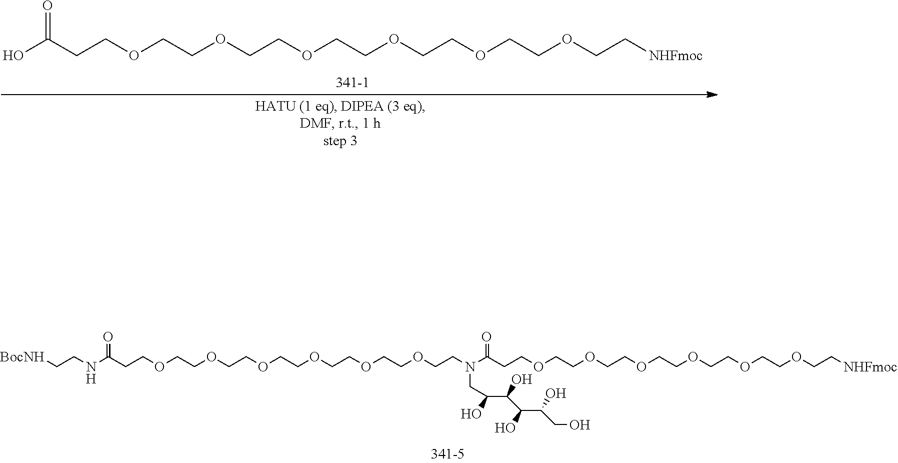

A solution of 341-4 (340 mg, 0.515 mmol), 341-1 (297 mg, 0.516 mmol) and HATU (196 mg, 0.515 mmol) in anhydrous DMF (10 mL) was stirred at room temperature for 5 min, then DIPEA (200 mg, 1.548 mmol) was added. The resulting solution was stirred for another 1 hr at r.t. until LCMS indicated complete reaction. The reaction solution was purified directly by reverse phase liquid chromatography to give 341-5 (475 mg, 0.390 mmol, 75.73%) as a white solid. purity=90%-95%.

### Step 5

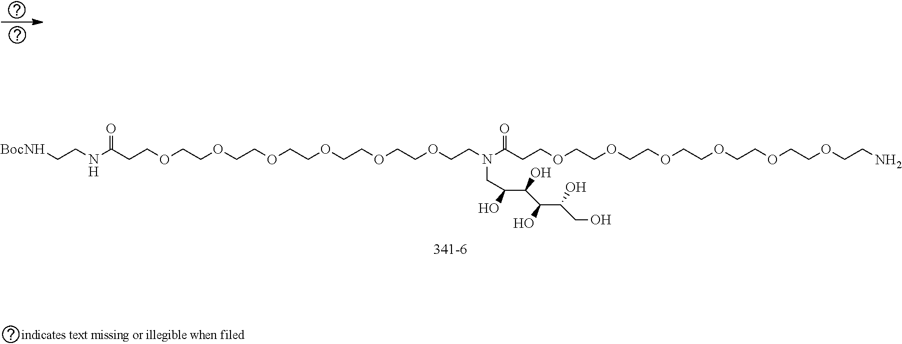 [SMILES: CC(C)(C)OC(=O)NCCNC(=O)CCOCCOCCOCCOCCOCCOCCN(C[C@H](O)[C@@H](O)[C@H](O)[C@H](O)CO)C(=O)CCOCCOCCOCCOCCOCCOCCN]

A solution of 341-5 (475 mg, 0.390 mmol) and DEA (2 mL) in anhydrous DMF (8 mL) was stirred at room temperature for 1 hr until LCMS indicated all starting amine was disappeared and desired product was detected. Then the solution was concentrated to dryness to 341-6 (372 mg, 0.374 mmol, 95.90%) as colorless oil, used as such in the next step. purity=90%-95%.

### Step 6

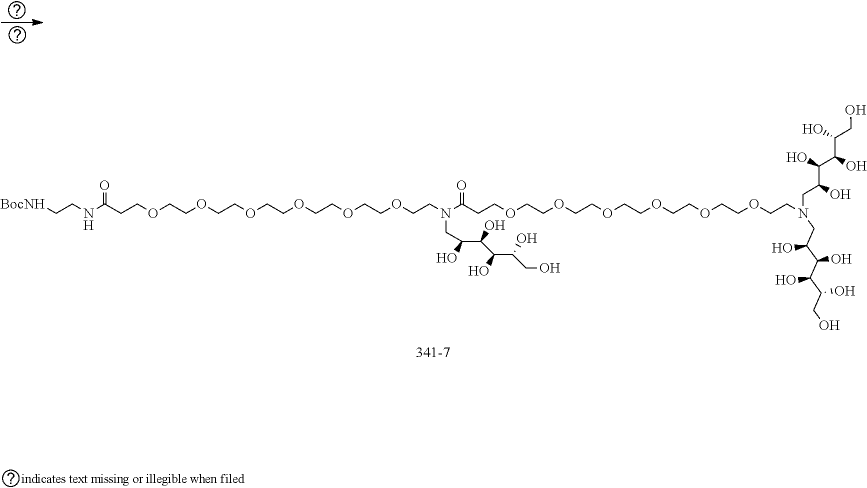 [SMILES: CC(C)(C)OC(=O)NCCNC(=O)CCOCCOCCOCCOCCOCCOCCN(C[C@H](O)[C@@H](O)[C@H](O)[C@H](O)CO)C(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 341-6 (372 mg, 0.374 mmol) and D-Glucose (337 mg, 1.871 mmol) in anhydrous Methanol (50 mL) was heated at 50° C. for 30 min, then NaCNBH₃ (118 mg, 1.878 mmol) was added. The resulting solution was stirred for another 6 hr at 70° C. until indicated all starting amine was disappeared and the mass of desired product was detected. Then the reaction solution was concentrated and purified by reverse phase liquid chromatography to give 341-7 (406 mg, 0.307 mmol, 82.09%) as a white solid. purity=90%-95%.

### Step 7

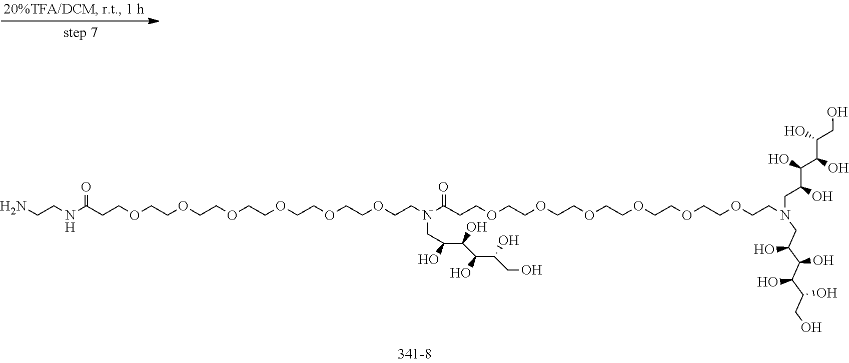 [SMILES: NCCNC(=O)CCOCCOCCOCCOCCOCCOCCN(C[C@H](O)[C@@H](O)[C@H](O)[C@H](O)CO)C(=O)CCOCCOCCOCCOCCOCCOCCN(C[C@H](O)[C@@H](O)[C@H](O)[C@H](O)CO)C[C@H](O)[C@@H](O)[C@H](O)[C@H](O)CO]

A solution of 341-7 (100 mg, 0.076 mmol) and TFA (2 mL) in anhydrous DCM (8 mL) was stirred at room temperature for 1 hr until LCMS indicated all starting amine was disappeared and desired product was detected. Then the solution was concentrated to dryness to 341-8 (85 mg, 0.069 mmol, 90.79%) as yellow oil, used as such in the next step. purity=90%-95%.

### Step 8

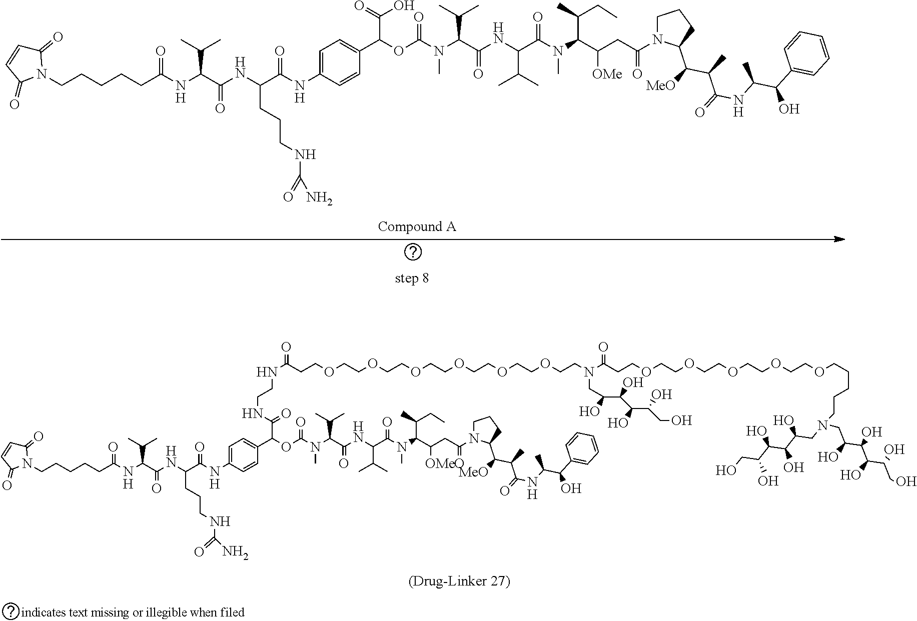

A solution of 341-8 (30 mg, 0.025 mmol), Compound A (33 mg, 0.025 mmol) and HATU (9 mg, 0.025 mmol) in anhydrous DMF (4 mL) was stirred at room temperature for 5 min, then DIPEA (10 mg, 0.074 mmol) was added. The resulting solution was stirred for another 1 hr at r.t. until LCMS indicated complete reaction. Then the reaction solution was purified by prep. HPLC to give drug-linker 27 (15 mg, 0.006 mmol, 24.01%) as a white solid. LCMS, m/z=856.38 (M/3+H)⁺.

### Example 28: Generation of Human Antibodies Against SLITRK6

The SLITRK6 extra-cellular domain (ECD) protein was prepared in house and used as antigen. Monoclonal antibodies against SLITRK6 were developed by sequentially immunizing mice with SLITRK6-his antigen proteins and an adjuvant, and the immunized animals included C57bl/6 and Balb/c mice. The animals were immunized with 100 μg of antigen per animal for the first shot, subsequently 50 μg of antigen per animal was used for immunization for the second and third shots, and 25 μg for the booster immunization. The immune adjuvant used in the experiments was PAP-1. AII animals were immunized by intraperitoneal injection. The mice with a good immunized titer were chosen for booster immunization.

After booster immunization, mice were sacrificed and soaked in 75% alcohol. The spleen was dissected out, ground with a grinding rod, and filtered through a cell strainer to prepare a single cell suspension. The spleen cell suspension was centrifuged at 1,500 rpm for 5 min, and the supernatant was discarded. 5 mL red blood cell lysate was added to lyse red blood cells at room temperature for 5 min and PBS was added to reach 20 mL. After centrifugation at 1,500 rpm for 5 min, the supernatant was discarded. Viable cells were counted after resuspension. Sp2/0 cells in a culture flask were collected and after centrifugation at 1,500 rpm for 5 min, the supernatant was discarded. Viable cells were counted after resuspension. The spleen cells were mixed with Sp2/0 cells at a ratio of 1.1:1 and subjected to centrifugation at 1,500 rpm for 5 min. The supernatant was discarded. The cells were resuspended in 20 mL electroporation buffer. After centrifugation at 1,500 rpm for 7 min, the supernatant was discarded and the step was repeated once.

The cells were resuspended with an appropriate amount of electroporation buffer to ensure the cell concentration of about 2×10⁷ cells/mL. The cell suspension was added to a 9 mL electroporation tank for fusion. After fusion, the cell suspension was transferred to 20 mL RPMI 1640 complete medium containing 20% FBS and then left at room temperature for 20 min. The fused cells were resuspended with RPMI 1640 medium containing 1×HAT, 1×BIOMYC3, and 20% FBS. The cell suspension was added to several 96-well cell culture plates at 100 uL/well to ensure that the cell volume per well was about 5×10⁴ cells/well, and the plates were placed in a 37° C. cell incubator. After 7 days, additional 100 μL of RPMI 1640 complete medium containing 20% FBS, 1×HAT, and 1×BIOMYC-3 was added to each well. After 10 days, the cell culture supernatants from hybridoma parent clones were collected and used for screening by binding to human and cynomolgus monkey SLITRK6 protein by ELISA. The ELSIA positive clones were subsequently chosen for FACS binding test and the cloneshu1H2-03, hu1H2-13, and hu14G1-03 were selected.

phA290 and phA108 were screened by phage display via a humanized single-domain antibody library. In the panning steps, solid-phase screening by immunotubes and liquid-phase screening by magnetic bead liquid processor were applied in an alternate manner. After 4 rounds of panning and enrichment, specific phages against SLITRK6-his antigen were eluted by trypsin and enriched. Certain enrichment of phage pools was validated by ELISA, and based on the results of ELISA primary screening, positive clones were selected for sequencing. Further selection for full-length construction was determined according to the diversity analysis. phA290 and phA108 were preferred via FACS binding and following bioactivity assay.

Sequences of the variable regions and CDRs of anti-SLITRK antibodies and prior-art antibody Sirtratuzumab are shown in Tables 1 and 2.

TABLE 1
Variable region sequence of anti-SLITRK6 antibodies

| Antibody | VH | VL |
|---|---|---|
| phA290 | qvqlvqsgaevkkpgasvkvsckasGYTFT | Eivmtqspgtlslspgeratlscrasqsvskyla |
| | SYYMHwvrqapgqgleWMGIINPSGG | wyqqrpgqaprlliydasnratgiparfsgsgsg |
| | STSYAQKFQGrvtmtrdtststvymelsslr | tdftltvsslepedfavyycqqrynwpltfgggttv |
| | sedtavyycARGFTRYFDYwgqgtlvtvs | eik (SEQ ID NO: 2) |
| | a (SEQ ID NO: 1) | |
| phA108 | qvqlvqsgaevkkpgasvkvsctasGYIFN | eivltqspatlslspgeratlscRASQSVSSY |
| | DYYLHwvrqapgqgleWVGIINPSGG | LAWYqqkpgqtprLLIYDASNRATgvpa |
| | SPVYAQKFQGrvtmtrdtstntvymelsslr | rfsgsgsgtdftltissletedfavyycQQRTN |
| | sedtavyycARGMVRGVRLFDYwgqg | WPLTfgggtkveik (SEQ ID NO: 10) |
| | tlvtvsa (SEQ ID NO: 9) | |
| hu1H2-03 | evalvesggglvqpggslrlscAASGFTFS | diqmtqspsslsasvgdrvtitcRTSENIHS |
| | DYGLHwvrqapgkglewvsYISSGSST | YLAwyqqkpgkSpQIIVyNAKTLADgvp |
| | VYFADTLKGrftisrdnaknslylqmnslrae | srfsgsgsgtdYtltisslqpedfGtFycQHF |
| | dtavyycTRGTWYFDVwgrgtlvtvss | WTTSRTfgggtkveik (SEQ ID NO: 18) |
| | (SEQ ID NO: 17) | |
| hu1H2-13 | evalvesggglvqpggsrlscAASGFTFS | diqmtqspsslsasvgdrvtitcRTSENIHS |
| | DYGLHwvrqapgkglewVAYISSGSST | YLAwyqqkpgkSpQIIVyNAKTLADgvp |
| | VYFADTLKGrftisrdnaknslylqmnslrae | srfsgsgsgtdYtltisslqpedfGtFycQHF |
| | dtavyycTRGTWYFDVwgrgtlvtvss | WTTSRTfgggtkveik (SEQ ID NO: 26) |
| | (SEQ ID NO: 25) | |
| hu14G1-03 | evqlvesggglvqpggslrlscAASGFTFS | diqmtqspsslsasvgdrvtitcRASGNIHS |
| | DYGMHwvrqapgkglewvsYISPGSST | YLAwyqqkpgkSpQIIVyNAKTLADgvp |
| | IYYADTMKGrftisrdnaknslylqmnslrae | srfsgsgsgtdYtltisslqpedfGtyycQHFW |
| | dtavyycARAYGGSYWYFAVwgqgtlvt | STPYTfgggtkleik (SEQ ID NO: 34) |
| | vss (SEQ ID NO: 33) | |
| Sirtratuzumab | QVQLVESGGGVVQPGRSLRLSCA | DIVMTQSPLSLPVTPGEPASISCRS |
| | ASGFTFSSYGMHWVRQAPGKGLE | SQSLLLSHGFNYLDWYLQKPGQS |
| | WVAVIWYDGSNQYYADSVKGRFTI | PQLLIYLGSSRASGVPDRFSGSGS |
| | SRDNSKNTLFLQMHSLRAEDTAVY | GTDFTLKISRVEAEDVGLYYCMQP |
| | YCARGLTSGRYGMDWGQGTTVT | LQIPWTFGQGTKVEIK |
| | VSS (SEQ ID NO: 41) | (SEQ ID NO: 42) |

TABLE 2
CDR sequences of anti-SLITRK6 antibodies

| Anti-body | HCDR1 | HCDR2 | HCDR3 | LCDR1 | LCDR2 | LCDR3 |
|---|---|---|---|---|---|---|
| phA290 | SYYMH | IINPSGG | GFTRYFD | RASQSV | DASNRAT | QQRYNW |
| | (SEQ ID | STSYAQK | Y (SEQ ID | SKYLA | (SEQ ID | PLT (SEQ |
| | NO: 3) | FQG | NO: 5) | (SEQ ID | NO: 7) | ID NO: 8) |
| | | (SEQ ID | | NO: 6) | | |
| | | NO: 4) | | | | |
| phA108 | DYYLH | IINPSGG | GMVRGV | RASQSV | DASNRAT | QQRTNW |
| | (SEQ ID | SPVYAQK | RLFDY | SSYLA | (SEQ ID | PLT (SEQ |
| | NO: 11) | FQG | (SEQ ID | (SEQ ID | NO: 15) | ID NO: |
| | | (SEQ ID | NO: 13) | NO: 14) | | 16) |
| | | NO: 12) | | | | |
| hu1H2-03 | DYGLH | YISSGSS | GTWYFD | RTSENIH | NAKTLAD | QHFWTT |
| | (SEQ ID | TVYFADT | V (SEQ ID | SYLA | (SEQ ID | SRT (SEQ |
| | NO: 19) | LKG (SEQ | NO: 21) | (SEQ ID | NO: 23) | ID NO: |
| | | ID NO: | | NO: 22) | | 24) |
| | | 20) | | | | |
| hu1H2-13 | DYGLH | YISSGSS | GTWYFD | RTSENIH | NAKTLAD | QHFWTT |
| | (SEQ ID | TVYFADT | V (SEQ ID | SYLA | (SEQ ID | SRT (SEQ |
| | NO: 27) | LKG (SEQ | NO: 29) | (SEQ ID | NO: 31) | ID NO: |
| | | ID NO: | | NO: 30) | | 32) |
| | | 28) | | | | |
| hu14G1-03 | DYGMH | YISPGSS | AYGGSY | RASGNIH | NAKTLAD | QHFWST |
| | (SEQ ID | TIYYADT | WYFAV | SYLA | (SEQ ID | PYT (SEQ |
| | NO: 35) | MKG | (SEQ ID | (SEQ ID | NO: 39) | ID NO: |
| | | (SEQ ID | NO: 37) | NO: 38) | | 40) |
| | | NO: 36) | | | | |
| Sirtratuzumab | SYGMH | VIWYDGS | GLTSGRY | RSSQSLL | LGSSRAS | MQPLQIP |
| | (SEQ ID | NQYYAD | GMD | LSHNYLD | (SEQ ID | WT (SEQ |
| | NO: 43) | SVKG | (SEQ ID | (SEQ ID | NO: 47) | ID NO: |
| | | (SEQ ID | NO: 45) | NO: 46) | | 48) |
| | | NO: 44) | | | | |

### Example 29: Affinity of Antibodies to SLITRK6 (BLI)

Binding kinetic of antibody-antigen was characterized by the bio-layer interferometry (BLI). 100 nM of SLITRK6′ extracellular domain linked to his tag was immobilized on anti-his biosensor tips (Fortebio). The sensors were rinsed with the buffer (50 mM Tris-AcOH, pH 6 or 7.5, 0.05% v/v surfactant P20) for 10 min to remove the unbound molecules, then dipped in the antibody solutions at different concentrations for 180 seconds and interspersed by a rinsing step in the buffer solution for 300 seconds. Binding affinity was calculated using ForteBio Data Acquisition 6.3 software (ForteBio) and was derived by fitting the kinetic data to a 1:1 Langmuir binding model utilizing global fitting algorithms. The dissociation or association equilibrium constant KD was calculated from the binding rate constants as KD=k_{off}/kₒₙ(where k_{off} and kₒₙ represent the dissociation kinetic constant and the association kinetic constant, respectively).

TABLE 3
Affinity results of antibodies to
human and cynomolgus monkey SLITRK6

| Immobilized | | KD | ka | kdis |
|---|---|---|---|---|
| Molecule | mAb | (M) | (1/Ms) | (1/s) |
| huSLITRK6 | phA290 | 1.06E−09 | 1.44E06 | 1.53E−03 |
| | phA108 | 1.05E−09 | 9.24E05 | 9.69E−04 |
| | hu1H2-03 | 9.20E−10 | 4.80E05 | 4.41E−04 |
| | hu1H2-13 | 9.16E−10 | 4.80E05 | 4.40E−04 |
| | hu14G1-03 | 2.12E−10 | 3.90E05 | 8.26E−05 |
| | Sirtratumab | 5.00E−09 | 8.31E05 | 4.16E−03 |
| cynoSLITRK6 | phA290 | 3.14E−09 | 1.11E06 | 3.49E−03 |
| | phA108 | 4.69E−09 | 8.09E05 | 3.79E−03 |
| | hu1H2-03 | 1.27E−09 | 3.48E05 | 4.44E−04 |
| | hu1H2-13 | 1.36E−09 | 3.76E05 | 5.13E−04 |
| | hu14G1-03 | 9.65E−10 | 3.41E05 | 3.29E−04 |
| | Sirtratumab | 6.01E−09 | 6.15E05 | 3.70E−03 |

hu14G1-03, hu1H2-03, hu1H2-13, phA290, and phA108 all showed high affinity to human SLITRK6 (huSLITRK6), compared to Sirtratumab. All candidates could cross-react with cynomolgus monkey SLITRK6 (cynoSLITRK6).

### Example 30: Epitope Binning of Antibodies

Epitope binning was characterized by the bio-layer interferometry (BLI). 100 nM of human SLITRK6′ extracellular domain linked to his tag was immobilized on anti-his biosensor tips (Fortebio). The sensors were rinsed with the buffer (50 mM Tris-AcOH, pH 6 or 7.5, 0.05% v/v surfactant P20) for 30 seconds to remove the unbound molecules, then dipped in the 100 nM of antibody 1 solutions for 180 seconds and interspersed by a rinsing step in the buffer solution for 300 seconds. After that, the sensors were dipped in the 100 nM of antibody 2 solutions. The epitope cluster was calculated using ForteBio Data Acquisition 6.3 software.

TABLE 4
Epitope binning results of antibodies

| | Ab2 | | | | | |
|---|---|---|---|---|---|---|
| | hu1H2- | hu1H2- | hu14G1- | | | |
| Ab1 | 03 | 13 | 03 | phA108 | phA290 | Sirtratumab |
| hu1H2-03 | 0.0113 | 0.0122 | 0.0077 | 0.3036 | 0.2988 | 0.2995 |
| hu1H2-13 | 0.0288 | 0.0284 | 0.0231 | 0.2991 | 0.3021 | 0.3121 |
| hu14G1-03 | 0.0244 | 0.0268 | 0.0192 | 0.3230 | 0.3258 | 0.2933 |
| phA108 | 0.2919 | 0.2852 | 0.2743 | 0.0346 | 0.0375 | 0.2761 |
| phA290 | 0.2685 | 0.2631 | 0.2540 | 0.0379 | 0.0426 | 0.25S1 |
| Sirtratumab | 0.2807 | 0.2825 | 0.2436 | 0.2523 | 0.2555 | 0.0645 |

*White background: Sharing similar epitope; Gray background: Sharing different epitope

According to the epitope binning results of antibodies, hu1H2-03, hu1H2-13 and hu14G1-03 were likely to share the similar epitope. phA290 and phA108 were likely to share the similar epitope. All candidates had different epitope from Sirtratumab.

### Example 31: Species Cross-Reaction of Antibodies by ELISA

First, a purified antigen was adsorbed to the surface of a PVC 96-well plate for ELISA at 4° C. overnight, the plate was washed twice by filling the wells with 350 μL TBST buffer (200 mM Sodium Chloride, 20 mM Tris, 0.05% Tween-20, pH 8). The surface of the solid phase having no antigen adsorbed thereto was covered with BSA. After washing the surface with TBST buffer, the surface was incubated with a serially diluted mAb as a primary antibody to bind to the antigen for 1 hour at room temperature. The plate was washed twice with TBST buffer and added an antibody labeled with an enzyme as a secondary antibody to incubate for 1 hour at room temperature. After washing, a substrate for the enzyme TMB was added and a change in absorbance which occurs due to color development induced by degradation of the substrate and the stopping solution was added to avoid the overdevelopment, then to read the optical density at 450 nm using a microplate reader (SpectraMax i3).

TABLE 5
Species cross-reaction results of antibodies

| | | | hu1H2- | hu1H2- | hu14G1- | Sirtra- |
|---|---|---|---|---|---|---|
| EC₅₀ (nM) | phA290 | phA108 | 03 | 13 | 03 | tumab |
| huSLITRK6 | 0.09 | 0.05 | 0.04 | 0.07 | 0.04 | 0.06 |
| cynoSLITRK6 | 0.06 | 0.06 | 0.05 | 0.05 | 0.04 | 0.06 |
| (97.6%) | | | | | | |
| ratSLITRK6 | 0.18 | 0.29 | NA | NA | NA | 0.88 |
| (88.0%) | | | | | | |

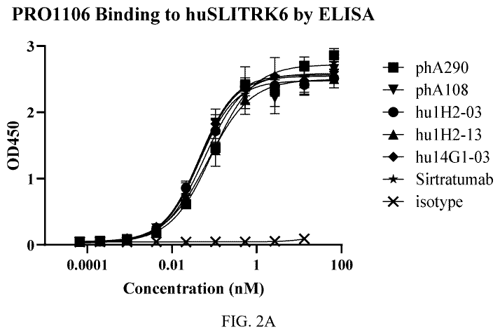

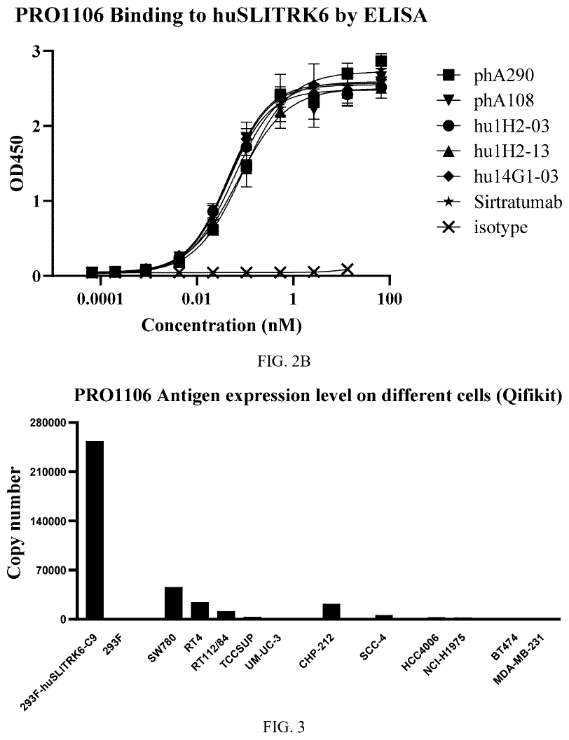

As shown in FIGS. 2A-2C and table 5 illustrating binding results of antibodies, all candidates showed high affinity to human and cynomolgus monkey SLITRK6.

### Example 32: Preparation of ADCs Conjugated with LD038

Antibodies phA290, phA108, hu1H2-03, hu1H2-13, hu14G1-03, and Sirtratumab were linked with drug-linker LD038 to obtain ADCs phA290-LD038 (8), phA108-LD038 (8), hu1H2-03-LD038 (8), hu1H2-13-LD038 (8), hu14G1-03-LD038 (8), and Sirtratumab-LD038 (8).

Specifically, 2 mL of each antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 10.0. The reducing reaction was conducted for 2 hours at 25° C. The excess TCEP and its byproduct were removed by ultrafiltration with pH=6.9 50 mM sodium phosphate buffer. LD038 (salt of TFA) was dissolved in water at a concentration of 20 mg/mL and added to reduced mAb at a molar ratio of 9.5 (LD038/mAb). The coupling reaction was stirred for 2 hours at 25° C. The excess LD038 and its impurities were removed by ultrafiltration with 50 mM sodium phosphate buffer. The ADC was stored in 20 mM histidine buffer containing 6% sucrose and 0.02% (w/V) Tween 20 by UFDF. The purity of each ADC as determined by SEC-HPLC was 98.0% and DAR value as determined by LC-MS was 8.0.

### Example 33: Copy Number of SLITRK6 on SLITRK6-Expressing Cell Lines

Flow cytometry was used for confirming SLITRK6 expression in cell lines using a QIFIKIT (DAKO, K0078). Specifically, cells were labeled with 10 μg/mL primary mouse monoclonal antibody (mu1H2) and left standing at 4° C. for 30 min. After washing twice with PBS containing 1% BSA, cells and Set-Up Beads and Calibration Beads of the kit were labeled, in parallel, with fluorescein-conjugated anti-mouse secondary antibody in PBS containing 1% BSA and left standing at 4° C. for 30 min. The cells and beads were washed twice with PBS containing 1% BSA, thereafter resuspended in PBS containing 1% BSA, and detected by using a flow cytometer (Beckman, Cytoflex). The fluorescence correlated with the number of bound primary antibody molecules on the cells and on the beads. The samples were then analyzed on the flow cytometer and copy number calculated based on the equation of the calibration curve. The results are illustrated in FIG. 3 and Table 6.

TABLE 6
Copy number of SLITRK6 on SLITRK6-expressing cell lines

| | | | Copy number |
|---|---|---|---|
| Cell name | Cell type | Level | (10³ per cell) |
| 293F-huSLITRK6-C9 | overexpressing | High | 253.7 |
| 293F | HEK | Negative | 0.4 |
| SW780 | BLCA | High | 46.0 |
| RT4 | BLCA | Moderate | 24.4 |
| RT112/84 | BLCA | Moderate | 11.7 |
| TCCSUP | BLCA | Low | 3.7 |
| UM-UC-3 | BLCA | Low | 1.9 |
| CHP-212 | GBMLGG | Moderate | 22.1 |
| SCC-4 | HNSC | Low | 6.2 |
| HCC4006 | LUAD | Low | 2.9 |
| NCI-H1975 | LUAD | Low | 2.7 |
| BT474 | BRCA | Low | 1.5 |
| MDA-MB-231 | BRCA | Negative | 0.0 |

### Example 34: Binding Activity of Antibodies to SLITRK6-Expressing Cells

The binding activity of mAbs was evaluated by flow cytometer (Beckman, Cytoflex). Specifically, cells were incubated with 50 μL of mAbs or ADCs in series dilutions in PBS containing 1% BSA at 4° C. for 30 min. The cells were washed twice with PBS containing 1% BSA, stained with PE-conjugated anti human Fc in PBS containing 1% BSA and then incubated at 4° C. for 20 min. After that the cells were washed twice with PBS and analyzed by a flow cytometer.

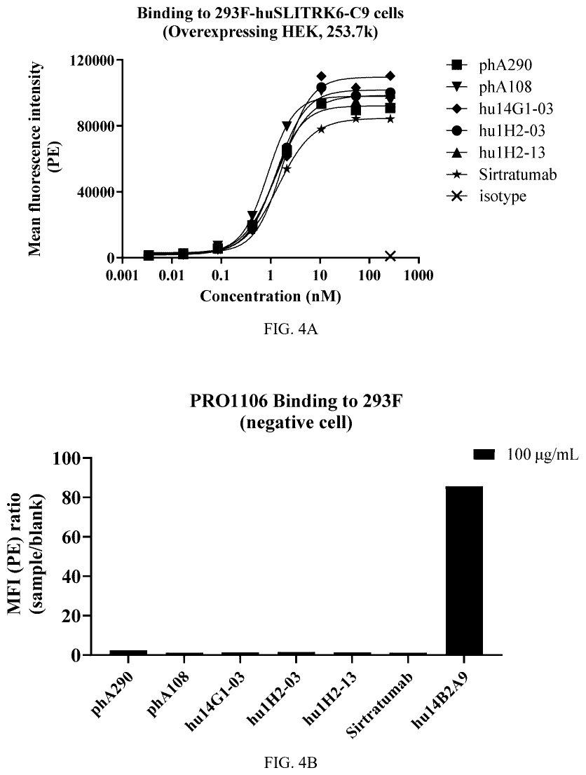

The binding activity of antibodies to 293F-huSLITRK6-C9 cells was evaluated and the results are illustrated in FIG. 4A and table 7. The binding activity of antibodies to 293F cells (negative cells) was evaluated and the results are illustrated in FIG. 4B. All candidates showed strong binding activity to human 293F-SLITRK6-expressing cell line with the EC₅₀ in the range of 0.87˜1.77 nM. All candidates showed no obvious binding to 293F cells.

TABLE 7
Binding activity of antibodies to 293F-huSLITRK6-C9 cells

| | | | hu1H2- | hu1H2- | hu14G1- | |
|---|---|---|---|---|---|---|
| ID | phA290 | phA108 | 03 | 13 | 03 | Sirtratumab |
| EC₅₀ (nM) | 1.16 | 0.87 | 1.33 | 1.32 | 1.77 | 1.41 |

### Example 35: Binding Activity of Antibodies to SLITRK6-Expressing Tumor Cells

The binding activity of mAbs was evaluated by flow cytometer (Beckman, Cytoflex). Specifically, cells were incubated with 50 μL of mAbs or ADCs in series dilutions in PBS containing 1% BSA at 4° C. for 30 minutes. The cells were washed twice with PBS containing 1% BSA, stained with PE-conjugated anti human Fc in PBS containing 1% BSA and then incubated at 4° C. for 20 min. After that the cells were washed twice with PBS and analyzed by a flow cytometer.

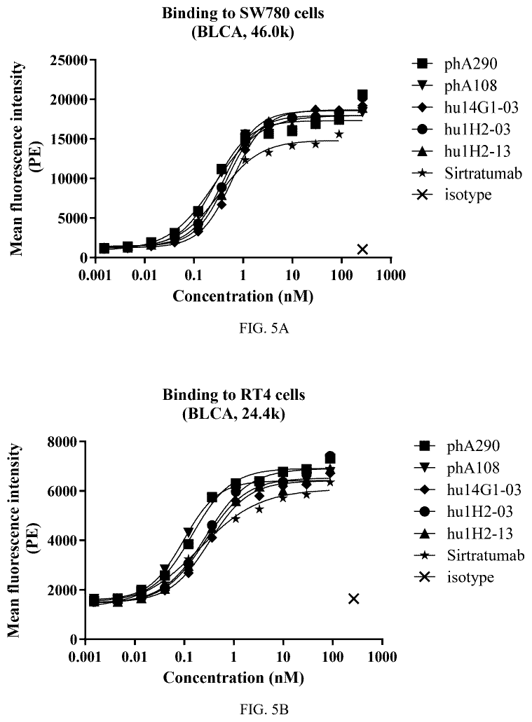

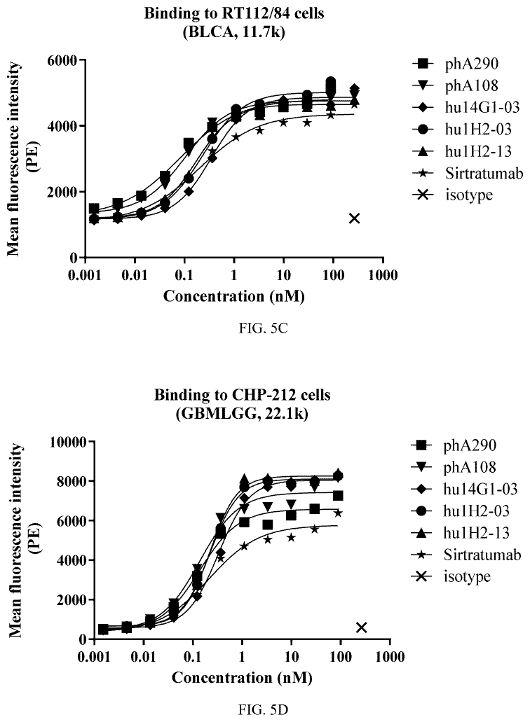

The binding activity of antibodies to SLITRK6-expressing tumor cell lines (SW780, RT4, RT112/84, CHP-212, and SCC-4) was evaluated. The copy number of SLITRK6 on the cell lines are as shown in Table 8. The results of binding activity are illustrated in FIGS. 5A-5E and Table 9. All candidates showed high binding activity to tumor cells with the EC₅₀ in the range of 0.08˜0.45 nM.

TABLE 8
Copy number of SLITRK6 on tumor cell lines

| Tumor cell lines | Copy number (10³ per cell) | Tumor type |
|---|---|---|
| SW780 | 46.0 | BLCA |
| RT4 | 24.4 | BLCA |
| RT112/84 | 11.7 | BLCA |
| CHP-212 | 22.1 | GBMLGG |
| SCC-4 | 6.2 | HNSC |

TABLE 9
Binding activity of antibodies to SLITRK6-expressing tumor cells

| | | | hu1H2- | hu1H2- | hu14G1- | |
|---|---|---|---|---|---|---|
| EC₅₀ (nM) | phA290 | phA108 | 03 | 13 | 03 | Sirtratumab |
| SW780 | 0.27 | 0.27 | 0.41 | 0.45 | 0.61 | 0.33 |
| RT4 | 0.14 | 0.10 | 0.28 | 0.27 | 0.32 | 0.20 |
| RT112/84 | 0.08 | 0.10 | 0.23 | 0.17 | 0.35 | 0.20 |
| CHP-212 | 0.14 | 0.14 | 0.22 | 0.22 | 0.33 | 0.21 |
| SCC-4 | 0.15 | 0.18 | 0.27 | 0.23 | 0.44 | NA |

### Example 36: Binding Activity of ADCs Conjugated with LD038 to SLITRK6-Expressing Tumor Cells

The binding activity of mAbs and ADCs was evaluated by flow cytometer (Beckman, Cytoflex). Specifically, cells were incubated with 50 μL of mAbs or ADCs in series dilutions in PBS containing 1% BSA at 4° C. for 30 min. The cells were washed twice with PBS containing 1% BSA, stained with PE-conjugated anti human Fc in PBS containing 1% BSA and then incubated at 4° C. for 20 min. After that the cells were washed twice with PBS and analyzed by a flow cytometer.

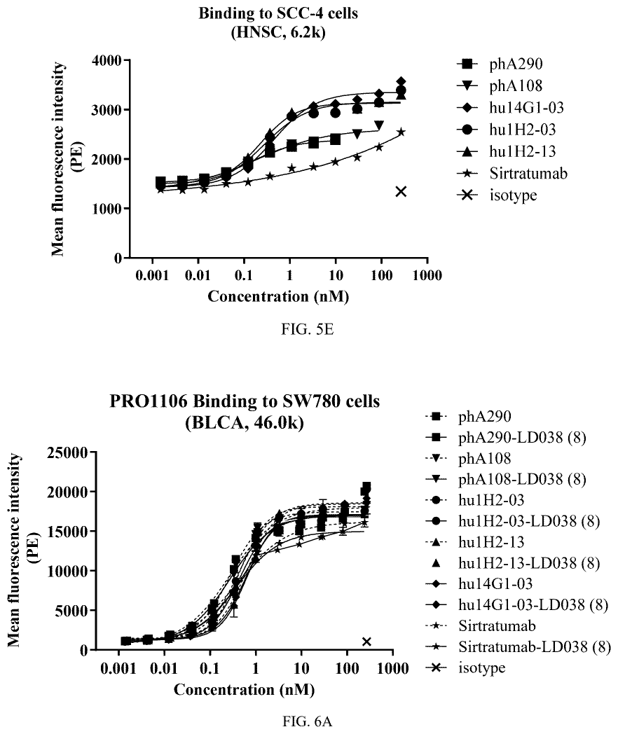

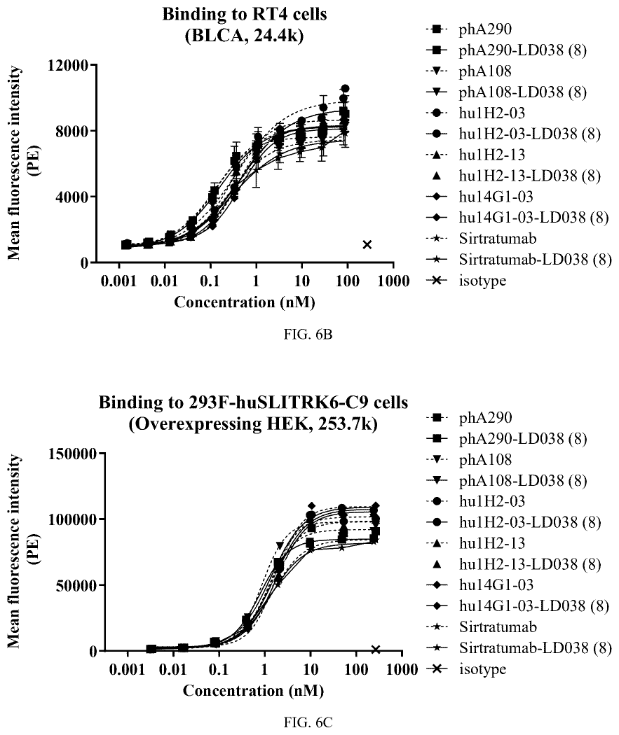

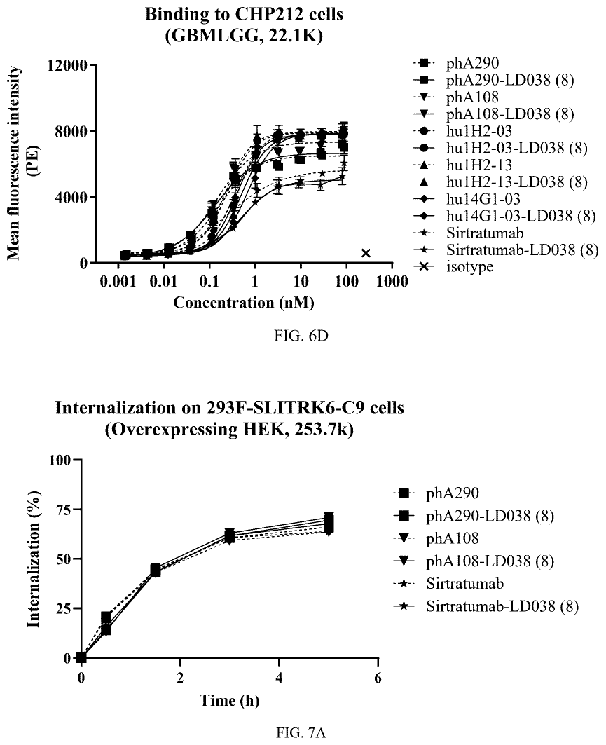

The binding activity of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) to SLITRK6-expressing cell lines (SW780, RT4, 293F-huSLITRK6-C9, and CHP-212) was evaluated. The copy number of SLITRK6 on the cell lines are as shown in Table 10. The results of binding activity are illustrated in FIGS. 6A-6D and Table 11. All candidates and their conjugates demonstrated strong binding activity to human SLITRK6-expressing cell lines. The binding activity of all candidates was not compromised after conjugation, except that of humanized candidates on CHP-212 cells.

TABLE 10
Copy number of SLITRK6 on tumor cell lines

| Tumor cell lines | Copy number (10³ per cell) | Tumor type |
|---|---|---|
| SW780 | 46.0 | BLCA |
| RT4 | 24.4 | BLCA |
| CHP-212 | 22.1 | GBMLGG |

TABLE 11
Binding activity of antibodies to SLITRK6-expressing cells

| | | | | | | | Sir- |
|---|---|---|---|---|---|---|---|
| | | | | hu1H2- | hu1H2- | hu14G1- | tra- |
| EC₅₀ (nM) | | phA290 | phA108 | 03 | 13 | 03 | tumab |
| SW780 | mAb | 0.27 | 0.27 | 0.44 | 0.44 | 0.62 | 0.39 |
| | ADC | 0.27 | 0.29 | 0.44 | 0.61 | 0.64 | 0.38 |
| RT4 | mAb | 0.14 | 0.12 | 0.36 | 0.27 | 0.38 | 0.26 |
| | ADC | 0.16 | 0.16 | 0.47 | 0.38 | 0.46 | 0.31 |
| 293F- | mAb | 1.16 | 0.87 | 1.33 | 1.32 | 1.77 | 1.41 |
| SLITRK6- | ADC | 0.79 | 1.09 | 1.35 | 1.60 | 1.79 | 1.32 |
| C9 | | | | | | | |
| CHP-212 | mAb | 0.16 | 0.14 | 0.24 | 0.22 | 0.37 | 0.25 |
| | ADC | 0.13 | 0.14 | 0.40 | 0.50 | 0.65 | 0.49 |

### Example 37: Internalization of Antibodies and ADCs Conjugated with LD038

The cells were incubated for 30 min at 4° C. with 20 μg/mL of antibody or ADC in PBS containing 0.1% BSA, then were washed at 4° C. to remove unbound material. The cells were incubated in 37° C. for different duration times and stained with PE-conjugated anti-human Fc for 30 min at 4° C. For 0 hour, the cells were kept at 4° C. The unbound antibody or ADC were washed with PBS containing 0.1% BSA at 4° C., and the cells were fixed with 2% paraformaldehyde and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time of 0 h, then divided by the MFI of cell surface-bound antibody at 4° C. at time of 0 h.

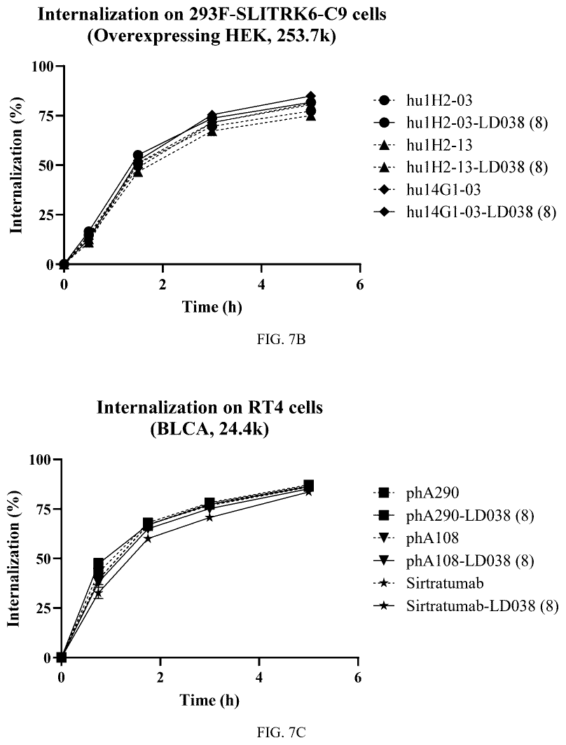

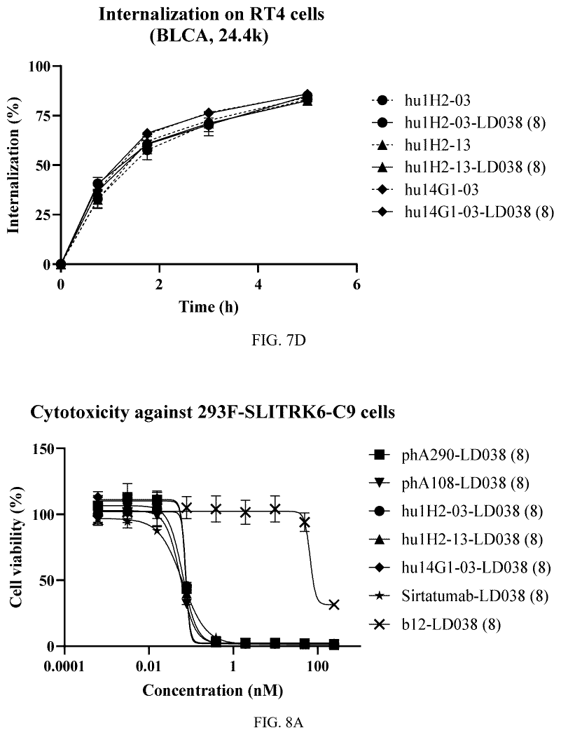

FIGS. 7A-7D illustrate internalization rates of antibodies (phA290, phA108, hu1H2-03, hu1H2-13, hu14G1-03, and Sirtratumab) and ADCs (phA290-LD038 (8), phA108-LD038 (8), hu1H2-03-LD038 (8), hu1H2-13-LD038 (8), hu14G1-03-LD038 (8), and Sirtratumab-LD038 (8)) in 293F-SLITRK6-C9 cells and RT4 cells.

### Example 38: Cytotoxicity of SLITRK6-Targeting ADCs Conjugated with LD038

The cells were prepared to plate into a −96-well solid white flat bottom plates for 24 hours in CO₂ thermostatic incubator with 5% CO₂ at 37° C. Then the cells were added a serially diluted ADC in the culture medium and incubated at 37° C. for 96 or 120 h. After the culture, the microplate was taken out from the incubator and left standing at room temperature for 30 min. The culture solution was charged with Cell Titer-Glo Luminescent Cell Viability Assay (Promega) for 5 min. The light emission was measured using a microplate reader (SpectraMax 3).

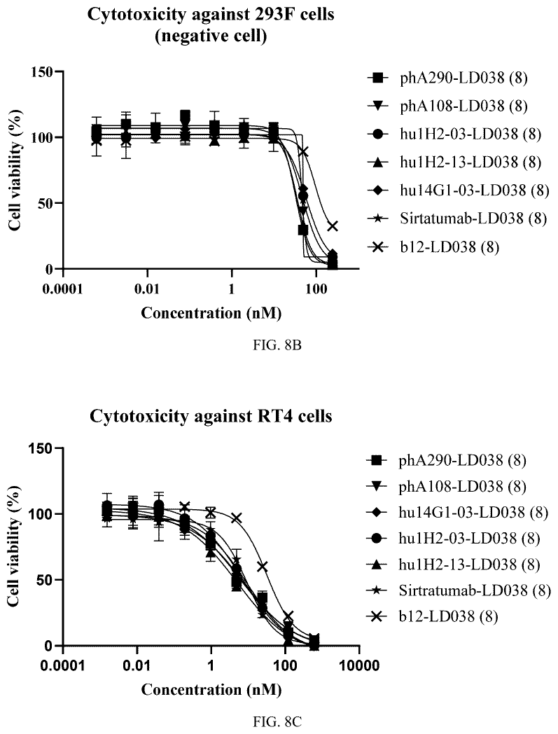

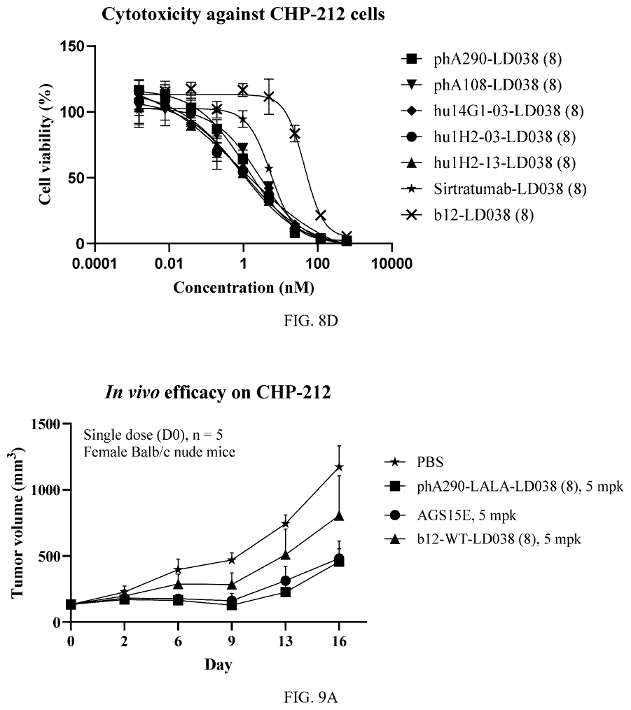

The results are as shown in FIGS. 8A-8D and Table 12. FIGS. 8A-8D illustrate cytotoxicity of ADCs with linker-drug LD038 on cell lines 293F-SLITRK6-C9 cells, 293F cells (negative cells), RT4 cells, and CHP-212 cells. As shown in FIGS. 8A-8D, all candidates conjugated with LD038 showed strong cytotoxicity on human SLITRK6-expressing cells, but no obvious cytotoxicity on negative cells.

TABLE 12
Cytotoxicity of ADCs conjugated with LD038

| | phA290- | phA108- | hu1H2-03- | hu1H2-13- | hu14G1-03- | Sirtratumab- |
|---|---|---|---|---|---|---|
| IC₅₀ (nM) | LD038 (8) | LD038 (8) | LD038 (8) | LD038 (8) | LD038 (8) | LD038 (8) |
| 293F-SLITRK6-C9 | NA | 0.06 | NA | 0.06 | NA | 0.06 |
| RT4 | 5.90 | 7.29 | 7.02 | 5.06 | 9.56 | 9.25 |
| CHP-212 | 1.22 | 2.82 | 0.83 | 0.93 | 0.91 | 5.56 |

### Example 39: In Vivo Efficacy of SLITRK6-Targeting ADCs

Balb/c nude mice were inoculated subcutaneously at right flank with different tumor cells for tumor development. When the tumor volume ranged from 100˜200 mm³, the animals were selected and assigned into different groups with 5 or 6 per group using matched distribution randomization based upon their tumor volumes. The treatments were started on randomization day (defined as DO) and were treated with different drugs. The tumor volume was measured twice per week during the observation period and TV>3000 mm³ was set as the humane endpoint.

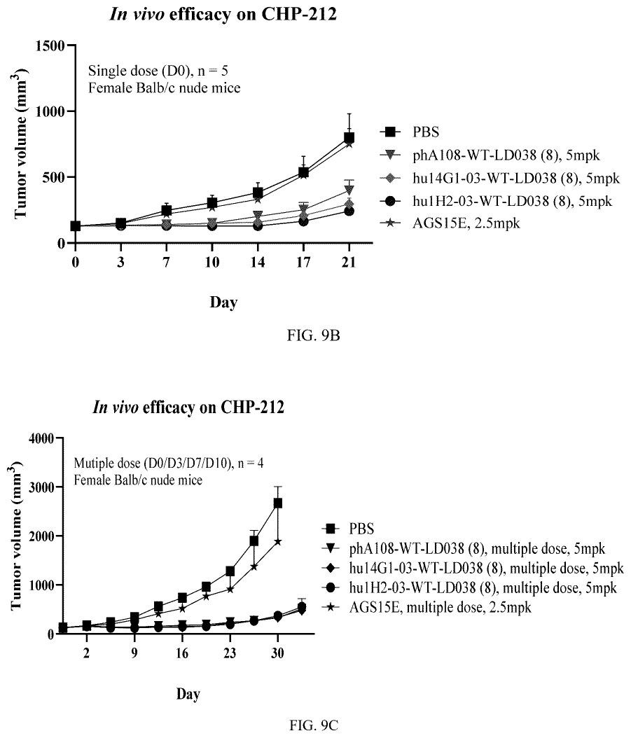

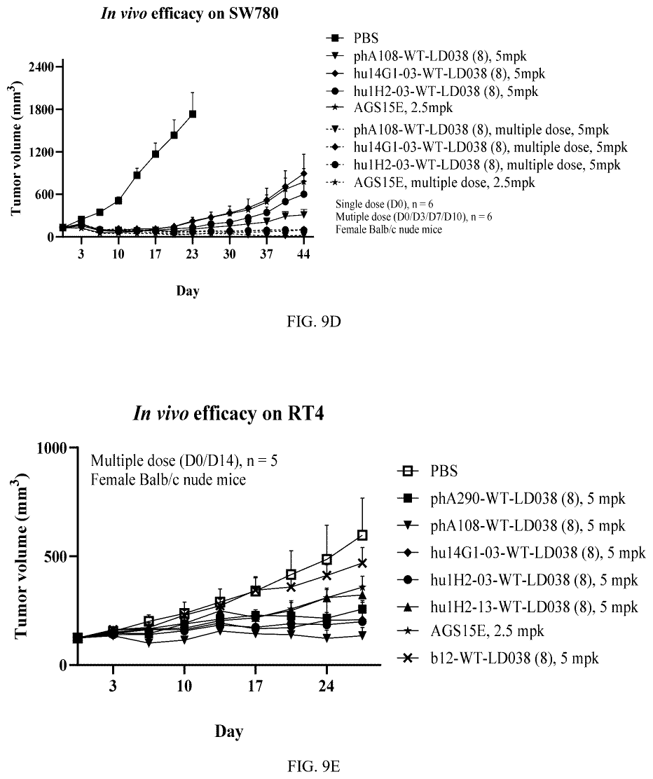

FIG. 9A is a graph illustrating in vivo efficacy of single dose of SLITRK6-targeting ADCs phA290-LALA-LD038 (8), AGS15E, b12-WT-LD038 on CHP-212; FIG. 9B is a graph illustrating in vivo efficacy of single dose of SLITRK6-targeting ADCs phA108-WT-LD038 (8), hu14G1-03-WT-LD038 (8), hu1H2-03-WT-LD038 (8), and AGS15E on CHP-212; FIG. 9C is a graph illustrating in vivo efficacy of multiple dose of SLITRK6-targeting ADCs phA108-WT-LD038 (8), hu14G1-03-WT-LD038 (8), hu1H2-03-WT-LD038 (8), and AGS15E on CHP-212; FIG. 9D is a graph illustrating in vivo efficacy of single dose and multiple dose of SLITRK6-targeting ADCs phA108-WT-LD038 (8), hu14G1-03-WT-LD038 (8), hu1H2-03-WT-LD038 (8), and AGS15E on SW780; and FIG. 9E is a graph illustrating in vivo efficacy of multiple dose of SLITRK6-targeting ADCs phA290-WT-LD038 (8), phA108-WT-LD038 (8), hu14G1-03-WT-LD038 (8), hu1H2-03-WT-LD038 (8), hu1H2-13-WT-LD038 (8), AGS15E, and b12-WT-LD038 (8) on RT4. As shown in FIGS. 9A-9E, all candidates showed strong in vivo efficacy on SLITRK6-expressing cells.

### Example 40: Affinity of Antibodies and ADCs to SLITRK6 and Family Members

In the following Examples, hu1H2-03 refers to antibody hu1H2-03 with a LALA mutation (hu1H2-03-LALA), unless indicated otherwise. Affinity of antibodies (hu1H2-03 and Sirtratumab) and their respective ADCs (hu1H2-03-LD038 and AGS15E) to SLITRK6 (Hu SLITRK6, Cyno SLITRK6, Mu SLITRK6, and Rat SLITRK6) and family members (Hu SLITRK1 and Hu SLITRK4) was tested. Binding kinetic of antibody-antigen was characterized by the bio-layer interferometry (BLI). 100 nM of SLITRK6′ extracellular domain linked to his tag was immobilized on anti-his biosensor tips (Fortebio). The sensors were rinsed with the buffer (50 mM Tris-AcOH, pH 6 or 7.5, 0.05% v/v surfactant P20) for 10 min to remove the unbound molecules, then dipped in the antibody solutions at different concentrations for 180 seconds and interspersed by a rinsing step in the buffer solution for 300 seconds. Binding affinity was calculated using ForteBio Data Acquisition 6.3 software (ForteBio) and was derived by fitting the kinetic data to a 1:1 Langmuir binding model utilizing global fitting algorithms. The dissociation or association equilibrium constant KD was calculated from the binding rate constants as KD=k_{off}/kₒₙ (where k_{off} and kₒₙ represent the dissociation kinetic constant and the association kinetic constant, respectively).

As shown in table 13, hu1H2-03 and hu1H2-03-LD038 showed high affinity and specificity in target binding.

TABLE 13
Affinity of antibodies and ADCs to SLITRK6 and family members

| | Hu | Cyno | Mu | Rat | Hu | Hu |
|---|---|---|---|---|---|---|
| | SLITRK6 | SLITRK6 | SLITRK6 | SLITRK6 | SLITRK1 | SLITRK4 |
| Analyte | K_{D} (nM) | | | | | |
| hu1H2-03 | 0.14 | 0.25 | 0.59 | NA | NA | NA |
| hu1H2-03- | 0.08 | 0.21 | 0.61 | NA | NA | NA |
| LD038 | | | | | | |
| Sirtratumab | 0.35 | 1.2 | 3.5 | 1.5 | NA | NA |
| AGS15E | 0.30 | 0.87 | 1.9 | 1.2 | NA | NA |

### Example 41: Binding Activity of Hu1H2-03-LD038 to to SLITRK6-Expressing Cells

The binding activity of antibodies (hu1H2-03 and Sirtratumab) and their respective ADCs (hu1H2-03-LD038 and AGS15E) was evaluated by flow cytometer (Beckman, Cytoflex). Specifically, cells were incubated with 50 μL of mAbs or ADCs in series dilutions in PBS containing 1% BSA at 4° C. for 30 min. The cells were washed twice with PBS containing 1% BSA, stained with PE-conjugated anti human Fc in PBS containing 1% BSA and then incubated at 4° C. for 20 min. After that the cells were washed twice with PBS and analyzed by a flow cytometer.

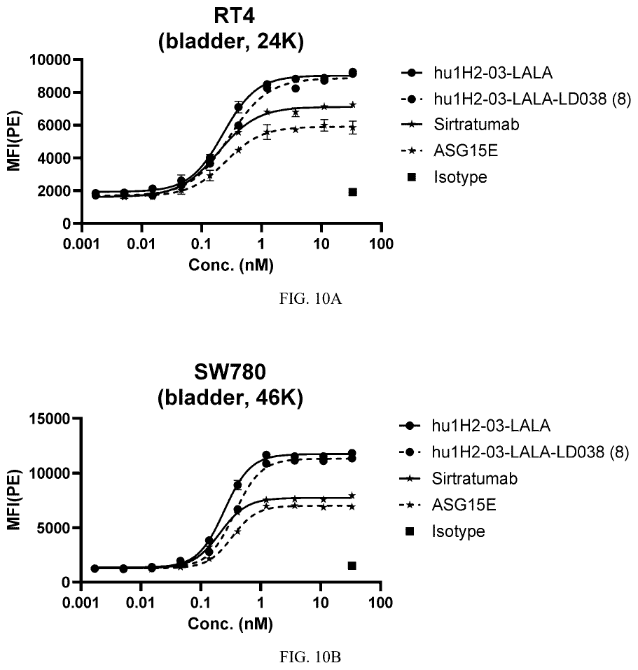

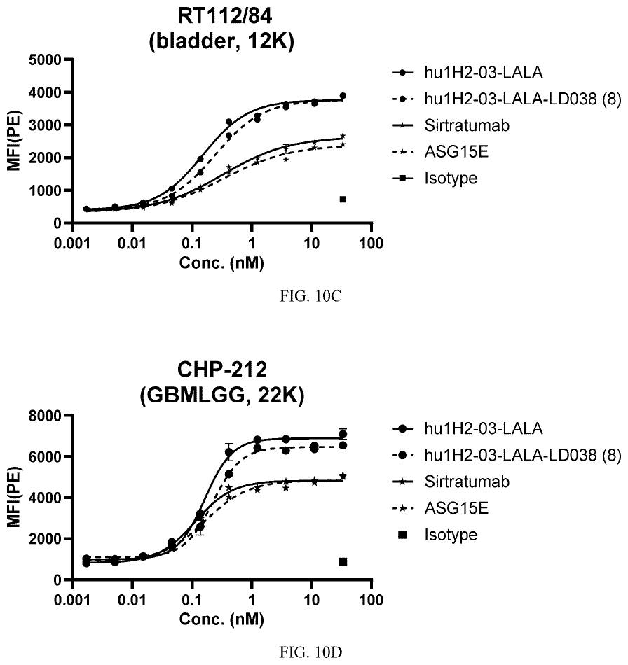

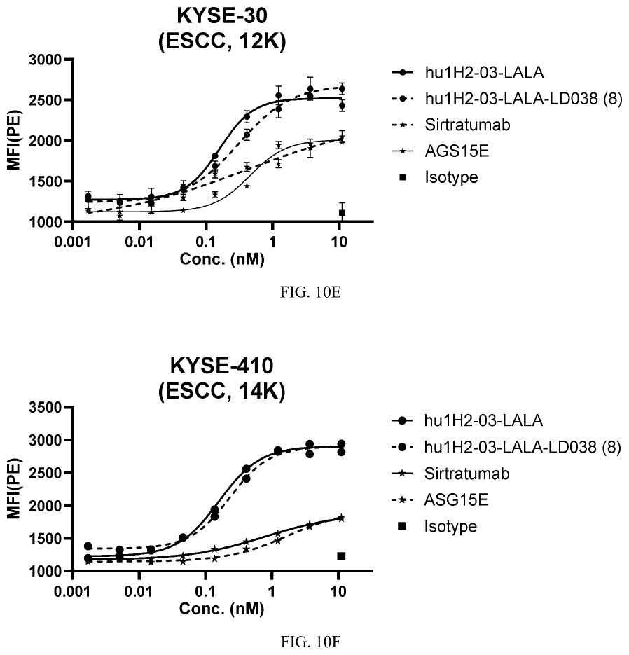

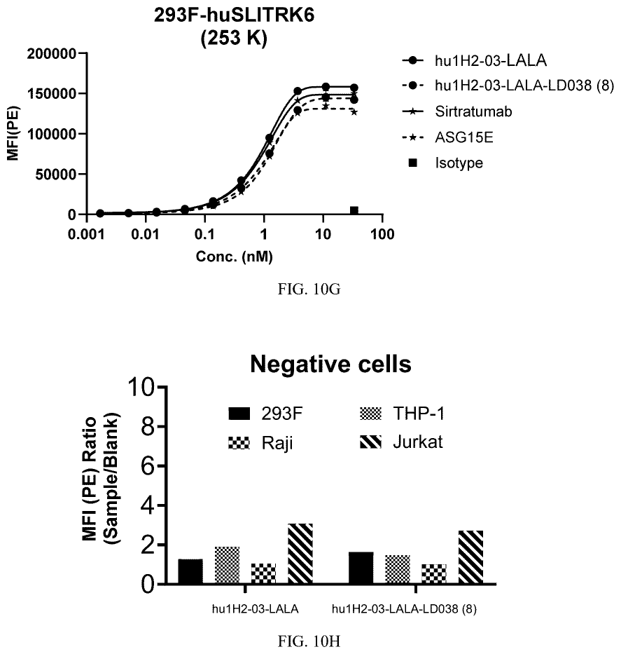

The binding activity of antibodies (hu1H2-03 and Sirtratumab) and their respective ADCs (hu1H2-03-LD038 and AGS15E) to SLITRK6-expressing cells, including RT4, SW780, RT112/84, CHP-212, KYSE-30, KYSE-410, and 293F-huSLITRK6, was evaluated and the results are illustrated in FIG. 10A-10G. Antibody hu1H2-03 and its ADC hu1H2-03-LD038 showed stronger binding activity to various SLITRK6-expressing cells than antibody Sirtratumab and its ADC AGS15E, respectively. The binding activity of antibody hu1H2-03 and its ADC hu1H2-03-LD038 to negative cells, 293F, THP-1, Raji, and Jurkat, was also evaluated and the results are illustrated in FIG. 10H. Antibody hu1H2-03 and its ADC hu1H2-03-LD038 showed no obvious binding to various negative cells.

### Example 42: PK in Rats and Monkeys

The hu1H2-03, hu1H2-03-LD038 (8), AGS15E, Sirtratumab were intravenously administered at 3 mg/kg to male Sprague Dawley rats (n=3 per group). Jugular vein blood was sampled from each rat at 10 min, 4 h, 1 d, 3 d, 7 d, 14 d and 21 d post dosing. Total antibody (TAb) concentration of hu1H2-03, hu1H2-03-LD038 (8), AGS15E, Sirtratumab in plasma was detected by goat anti-human IgG Fc (HRP), calculated using GraphPad Prism 8.0 software. The Tab (total antibody), ac-exatecan (antibody-conjugated exatecan), u-exatecan (unconjugated exatecan) were intravenously administered at 5 mg/kg to cyno monkeys (n=2). Jugular vein blood was sampled from each monkey at 10 min, 4 h, 1 d, 3 d, 7 d, 14 d and 21 d post dosing. Concentrations of the TAb in plasma were detected by goat anti-human IgG Fc (HRP), ac-exatecan, and u-exatecan in plasma were detected via LC-MS and calculated using GraphPad Prism 8.0 software.

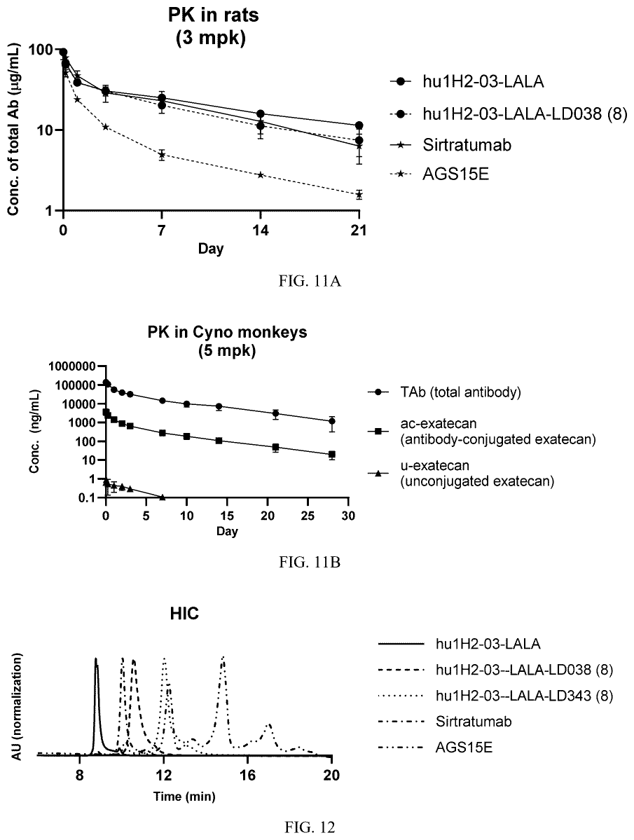

Plasma PKs of hu1H2-03-LD038 and hu1H2-03 in rats (n=3/group) and of hu1H2-03-LD038 in monkeys (n=2) are shown in FIGS. 11A-11B.

### Example 43: Preparation Hu1H2-03-LD343

Antibody hu1H2-03 was linked with drug-linker LD343 to obtain ADC hu1H2-03-LD343 (8). Specifically, 2 mL of the antibody (10 mg/mL) in 50 mM sodium phosphate buffer containing 5 mM EDTA (pH=6.9) was added to the aqueous of 10 mM TCEP HCl (Tris(2-carboxyethyl) phosphine HCl), at the molar ratio of TCEP to mAb is 10.0. The reducing reaction was conducted for 2 hours at 25° C. The excess TCEP and its byproduct were removed by ultrafiltration with pH=6.9 50 mM sodium phosphate buffer. LD343 (salt of TFA) was dissolved in water at a concentration of 20 mg/mL and added to reduced mAb at a molar ratio of 9.5 (LD343/mAb). The coupling reaction was stirred for 2 hours at 2₅° C. The excess LD343 and its impurities were removed by ultrafiltration with 50 mM sodium phosphate buffer. The ADC was stored in 20 mM histidine buffer containing 6% sucrose and 0.02% (w/V) Tween 20 by UFDF. The purity of each ADC as determined by SEC-HPLC was 98.0% and DAR value as determined by LC-MS was 8.0.

### Example 44: Hydrophilicity of Hu1H2-03-LD038 and Hu1H2-03-LD343

The hydrophobicity of test articles was measured in Waters® e2695 Separations Module with UV detection at 280 nm using TSKgel Butyl-NPR column (Tosoh Bioscience, TO0042168). The mobile phase A is 50 mM phosphate buffer, 1.5 M ammonium sulfate, pH 7.0. Mobile phase B is 50 mM phosphate buffer pH 7.0, 20% isopropanol. The flow rate is 0.5 mL/min at room temperature. Use Empower™ 3 SOFTWARE to analyze the peak retention time.

As shown in FIG. 12, there are markedly improved hydrophilicity and homogeneity for hu1H2-03-LD038 and hu1H2-03-LD343 when compared to the benchmark, AGS15E.

### Example 45: Binding Activity of Hu1H2-03-LD343 to SLITRK6-Expressing Tumor Cells

The binding activity of mAbs and ADCs was evaluated by flow cytometer (Beckman, Cytoflex). Specifically, cells were incubated with 50 μL of mAbs or ADCs in series dilutions in PBS containing 1% BSA at 4° C. for 30 min. The cells were washed twice with PBS containing 1% BSA, stained with PE-conjugated anti human Fc in PBS containing 1% BSA and then incubated at 4° C. for 20 min. After that the cells were washed twice with PBS and analyzed by a flow cytometer.

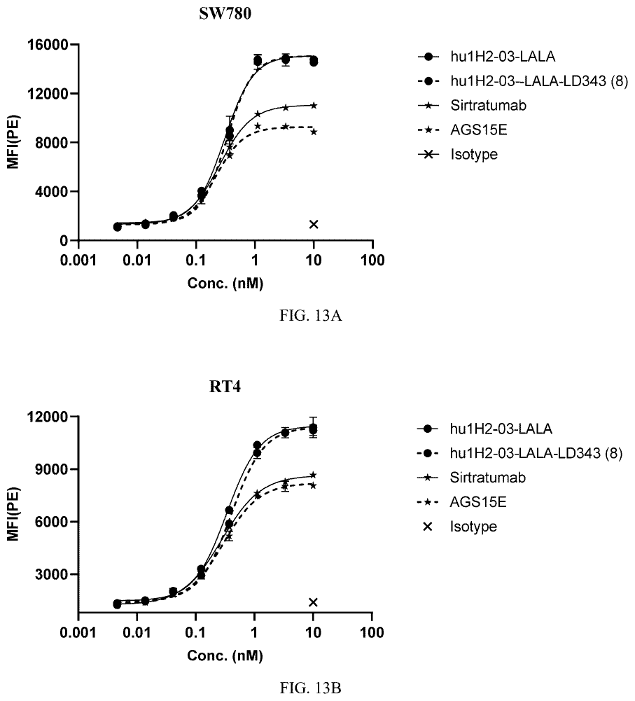

The binding activity of antibodies (hu1H2-03 and Sirtratumab) and their respective ADCs (hu1H2-03-LD343 and AGS15E) to SLITRK6-expressing cell lines (SW780 and RT4) was evaluated and the results are illustrated in FIGS. 13A-13B. Antibody hu1H2-03 and its ADC hu1H2-03-LD343 showed stronger binding activity to SW780 cells and RT4 cells than antibody Sirtratumab and its ADC AGS15E, respectively.

### Example 46: Internalization of hu1H2-03-LD038 and hu1H2-03-LD343

The cells were incubated for 30 min at 4° C. with 20 μg/mL of antibody or ADC in PBS containing 0.1% BSA, then were washed at 4° C. to remove unbound material. The cells were incubated in 37° C. for different duration times and stained with PE-conjugated anti-human Fc for 30 min at 4° C. For 0 hour, the cells were kept at 4° C. The unbound antibody or ADC were washed with PBS containing 0.1% BSA at 4° C., and the cells were fixed with 2% paraformaldehyde and analyzed by flow cytometry. Internalization rate was calculated by subtracting the mean fluorescence intensity (MFI) of cell surface-bound antibody at 37° C. at each timepoint from the MFI of cell surface-bound antibody at 4° C. at time of 0 h, then divided by the MFI of cell surface-bound antibody at 4° C. at time of 0 h.

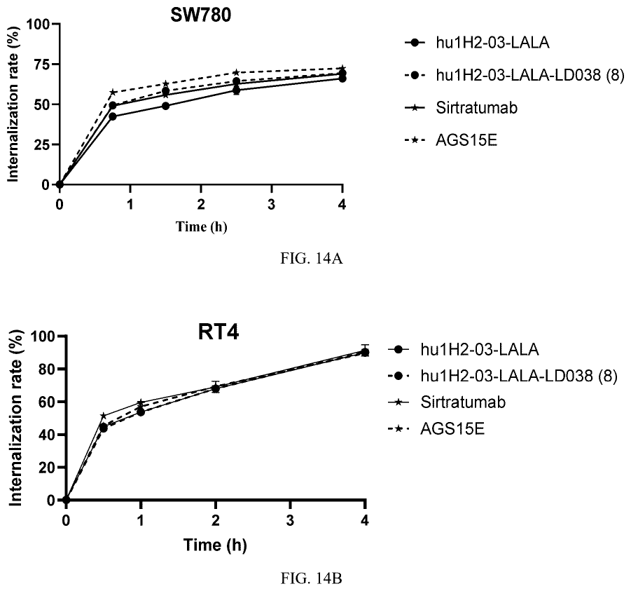

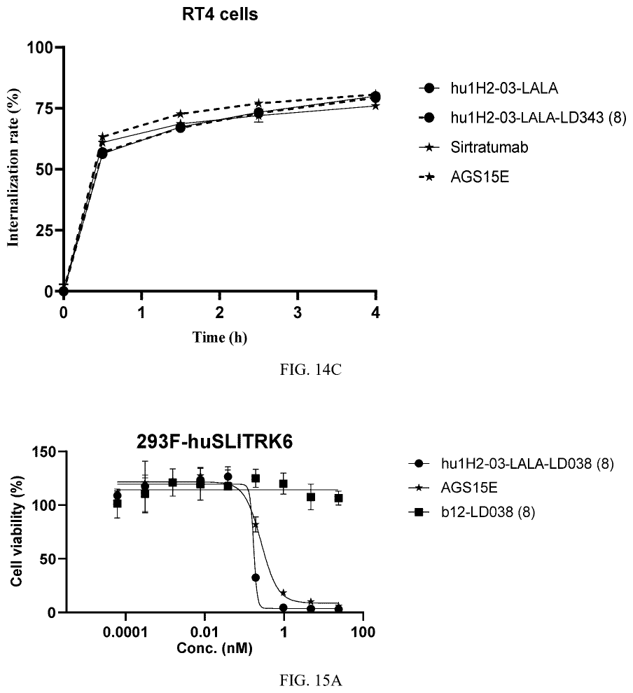

FIG. 14A illustrates internalization rates of antibodies (hu1H2-03 and Sirtratumab) and their respective ADCs (hu1H2-03-LD038 and AGS15E) in SW780 cells. FIG. 14B illustrates internalization rates of antibodies (hu1H2-03 and Sirtratumab) and their respective ADCs (hu1H2-03-LD038 and AGS15E) in RT4 cells. FIG. 14C illustrates internalization rates of antibodies (hu1H2-03 and Sirtratumab) and their respective ADCs (hu1H2-03-LD343 and AGS15E) in RT4 cells.

### Example 47: Cytotoxicity of hu1H2-03-LD038 and hu1H2-03-LD343

The cells were prepared to plate into a −96-well solid white flat bottom plates for 24 hours in CO₂ thermostatic incubator with 5% CO₂ at 37° C. Then the cells were added a serially diluted ADC in the culture medium and incubated at 37° C. for 96 or 120 h. After the culture, the microplate was taken out from the incubator and left standing at room temperature for 30 min. The culture solution was charged with Cell Titer-Glo Luminescent Cell Viability Assay (Promega) for 5 min. The light emission was measured using a microplate reader (SpectraMax 3).

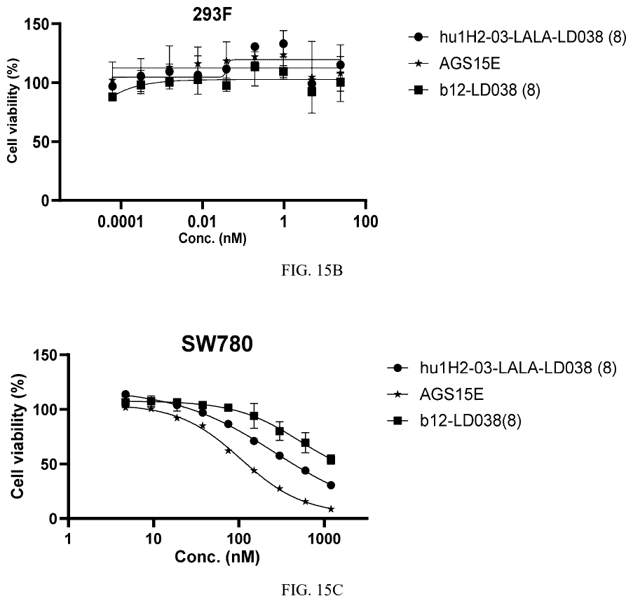

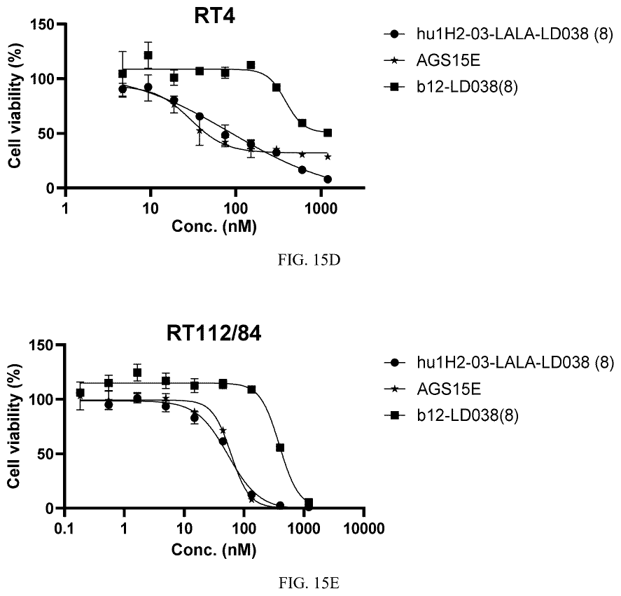

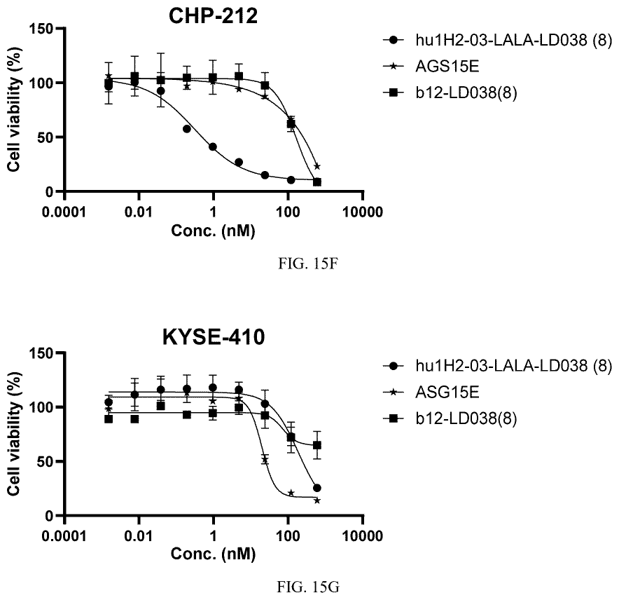

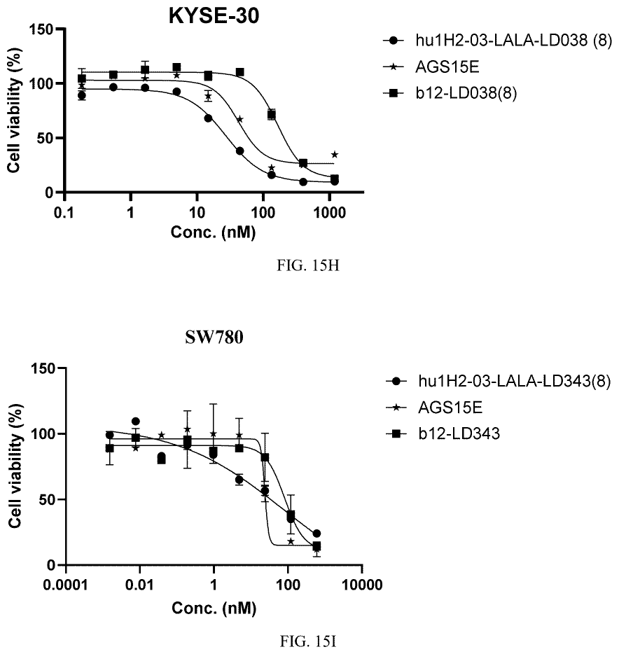

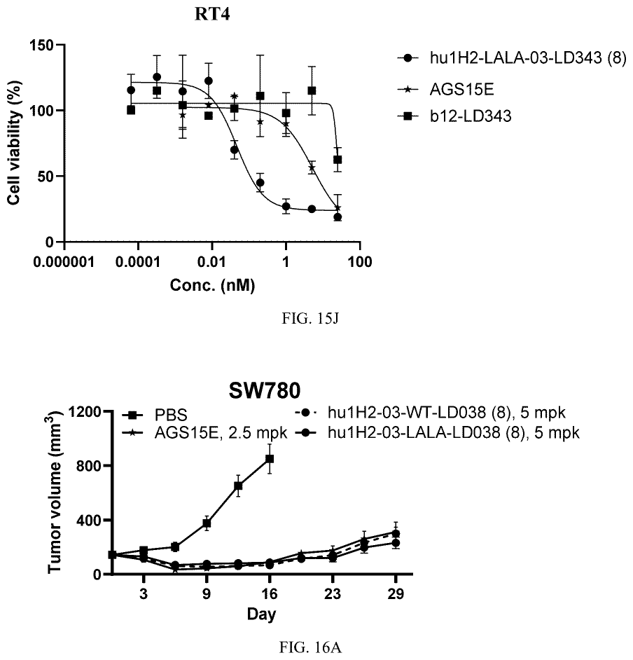

FIGS. 15A-15H illustrate cytotoxicity of hu1H2-03-LD038, AGS15E, and b12-LD038 on SLITRK6-expressing cells, including 293F-huSLITRK6, SW780, RT4, RT112/84, CHP-212, KYSE-410, and KYSE-30 as well as negative cell 293F. hu1H2-03-LD038 showed stronger or comparable cytotoxicity on human SLITRK6-expressing cells, compared to AGS15E. FIGS. 15I-15J illustrate cytotoxicity of hu1H2-03-LD343 on SW780 cells and RT4 cells. hu1H2-03-LD343 showed stronger cytotoxicity on human SLITRK6-expressing cells than AGS15E.

### Example 48: In Vivo Efficacy of Hu1H2-03-LD038 and Hu1H2-03-LD343

Balb/c nude mice were inoculated subcutaneously at right flank with different tumor cells for tumor development. When the tumor volume ranged from 100˜200 mm³, the animals were selected and assigned into different groups with 5 or 6 per group using matched distribution randomization based upon their tumor volumes. The treatments were started on randomization day (defined as DO) and were treated with different drugs. The tumor volume was measured twice per week during the observation period and TV>3000 mm³ was set as the humane endpoint.

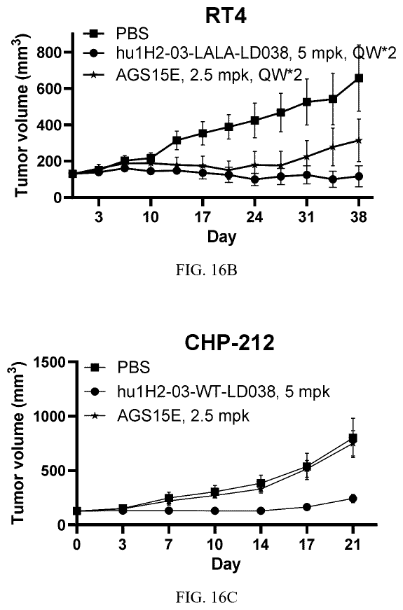

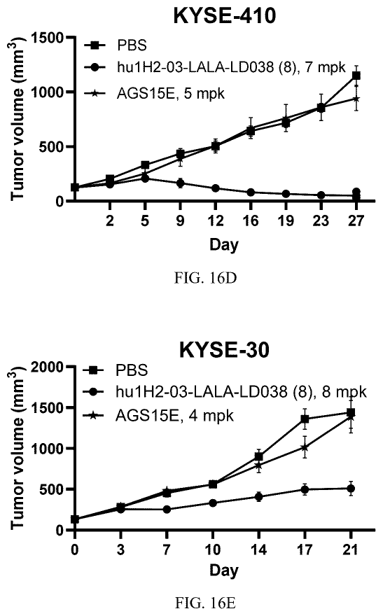

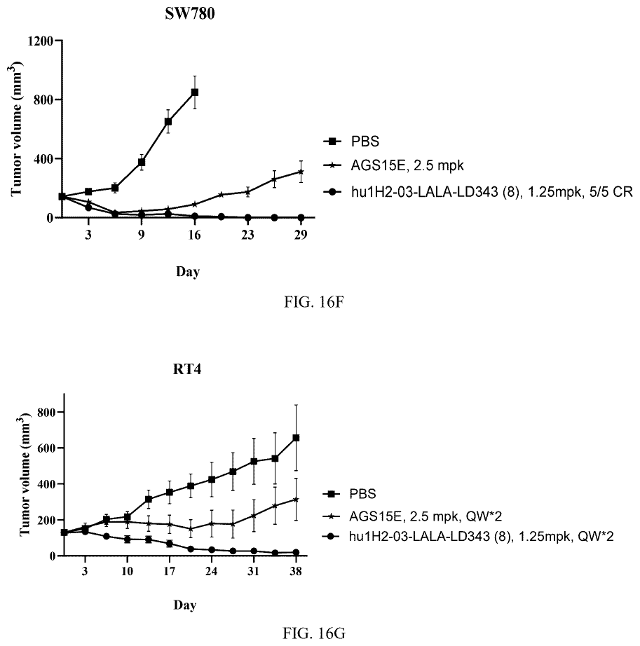

FIG. 16A illustrates in vivo efficacy of single dose of hu1H2-03-WT-LD038, hu1H2-03-LALA-LD038, and AGS15E on SW780 cells. FIG. 16B illustrates in vivo efficacy of single dose of hu1H2-03-LALA-LD038 and AGS15E on RT4 cells. FIG. 16C illustrates in vivo efficacy of single dose of hu1H2-03-WT-LD038 and AGS15E on CHP-212 cells. FIG. 16D illustrates in vivo efficacy of single dose of hu1H2-03-LD038 and AGS15E on KYSE-410 cells. FIG. 16E illustrates in vivo efficacy of single dose of hu1H2-03-LD038 and AGS15E on KYSE-30 cells. hu1H2-03-LD038 showed stronger or comparable in vivo efficacy on SLITRK6-expressing cells, compared to AGS15E. FIG. 16F illustrates in vivo efficacy of single dose of hu1H2-03-LD343 and AGS15E on SW780 cells. FIG. 16G illustrates in vivo efficacy of single dose of hu1H2-03-LD343 and AGS15E on RT4 cells. hu1H2-03-LD343 showed stronger in vivo efficacy on SLITRK6-expressing cells, compared to AGS15E. Therefore, both hu1H2-03-LD038 and hu1H2-03-LD343 showed potent antitumor activity in xenograft models representing bladder, esophageal cancer, and glioma.

### Example 49: In Vivo Efficacy of ADCs Conjugated with LD038 or LD343 in PDX Model

Balb/c nude mice were inoculated subcutaneously at right flank with patient bladder cells for tumor development. When the tumor volume ranged from 100-200 mm3, the animals were selected and assigned into different groups with 2 or 3 per group using matched distribution randomization based upon their tumor volumes. The treatments were started on randomization day (defined as DO) and were treated with different drugs. The tumor volume was measured twice per week during the observation period and TV>3000 mm3 was set as the humane endpoint.

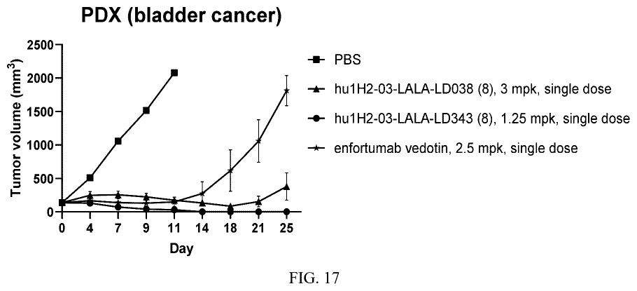

FIG. 17 illustrates in vivo efficacy of single dose of hu1H2-03-LD038, hu1H2-03-LD343, and enfortumab vedotin. hu1H2-03-LD038 and hu1H2-03-LD343 showed stronger in vivo efficacy in PDX models, compared to enfortumab vedotin.

Having thus described the basic concepts, it may be rather apparent to those skilled in the art after reading this detailed disclosure that the foregoing detailed disclosure is intended to be presented by way of example only and is not limiting. Various alterations, improvements, and modifications may occur and are intended to those skilled in the art, though not expressly stated herein. These alterations, improvements, and modifications are intended to be suggested by this disclosure and are within the spirit and scope of the exemplary embodiments of this disclosure.

Moreover, certain terminology has been used to describe embodiments of the present disclosure. For example, the terms “one embodiment,” “an embodiment,” and “some embodiments” mean that a particular feature, structure or characteristic described in connection with the embodiment is included in at least one embodiment of the present disclosure. Therefore, it is emphasized and should be appreciated that two or more references to “an embodiment” or “one embodiment” or “an alternative embodiment” in various portions of this specification are not necessarily all referring to the same embodiment. Furthermore, the particular features, structures or characteristics may be combined as suitable in one or more embodiments of the present disclosure.

Further, it will be appreciated by one skilled in the art, aspects of the present disclosure may be illustrated and described herein in any of a number of patentable classes or context including any new and useful process, machine, manufacture, or composition of matter, or any new and useful improvement thereof.

Furthermore, the recited order of processing elements or sequences, or the use of numbers, letters, or other designations, therefore, is not intended to limit the claimed processes and methods to any order except as may be specified in the claims. Although the above disclosure discusses through various examples what is currently considered to be a variety of useful embodiments of the disclosure, it is to be understood that such detail is solely for that purpose and that the appended claims are not limited to the disclosed embodiments, but, on the contrary, are intended to cover modifications and equivalent arrangements that are within the spirit and scope of the disclosed embodiments.

Similarly, it should be appreciated that in the foregoing description of embodiments of the present disclosure, various features are sometimes grouped together in a single embodiment, figure, or description thereof to streamline the disclosure aiding in the understanding of one or more of the various embodiments. This method of disclosure, however, is not to be interpreted as reflecting an intention that the claimed subject matter requires more features than are expressly recited in each claim. Rather, claim subject matter lie in less than all features of a single foregoing disclosed embodiment.

1. A binding agent which binds to SLIT and NTRK-like protein 6 (SLITRK6) comprising:
a heavy chain variable (VH) region and a light chain variable (VL) region, the VH region comprising complementarity determining regions HCDR1, HCDR2 and HCDR3 disposed in heavy chain variable region framework regions and the VL region comprising LCDR1, LCDR2 and LCDR3 disposed in light chain variable region framework regions, the VH and VL CDRs comprising amino acids sequences selected from the sets of amino acid sequences set forth in the group consisting of:
(i) SEQ ID NO: 19, SEQ ID NO: 20, SEQ ID NO: 21, SEQ ID NO: 22, SEQ ID NO: 23, and SEQ ID NO: 24, respectively;
(ii) SEQ ID NO: 11, SEQ ID NO: 12, SEQ ID NO: 13, SEQ ID NO: 14, SEQ ID NO: 15 and SEQ ID NO: 16, respectively;
(iii) SEQ ID NO: 3, SEQ ID NO: 4, SEQ ID NO: 5, SEQ ID NO: 6, SEQ ID NO: 7 and SEQ ID NO: 8, respectively;
(iv) SEQ ID NO: 27, SEQ ID NO: 28, SEQ ID NO: 29, SEQ ID NO: 30, SEQ ID NO: 31 and SEQ ID NO: 32, respectively; and
(v) SEQ ID NO: 35, SEQ ID NO: 36, SEQ ID NO: 37, SEQ ID NO: 38, SEQ ID NO: 39 and SEQ ID NO: 40, respectively.

2. The binding agent of claim 1, wherein the VH and VL regions comprise amino acid sequences that are selected from the pairs of amino acid sequences set forth in the group consisting of:
(i) SEQ ID NO: 17 and SEQ ID NO: 18, respectively;
(ii) SEQ ID NO: 9 and SEQ ID NO: 10, respectively;
(iii) SEQ ID NO: 1 and SEQ ID NO: 2, respectively;
(iv) SEQ ID NO: 25 and SEQ ID NO: 26, respectively; and
(v) SEQ ID NO: 33 and SEQ ID NO: 34, respectively.

3-6. (canceled)

7. The binding agent of claim 1, wherein the binding agent is an antibody or an antigen-binding portion thereof.

8. The binding agent of claim 1, wherein the binding agent is a monoclonal antibody, a Fab, a Fab′, an F(ab′), an Fv, a disulfide linked Fc, a scFv, a single domain antibody, a diabody, a bi-specific antibody, or a multi-specific antibody.

9. The binding agent of claim 1, wherein the heavy chain variable region further comprises a heavy chain constant region.

10. (canceled)

11. The binding agent of claim 9, wherein the heavy chain constant region is an IgG1 or IgG4 constant region.

12. (canceled)

13. The binding agent of claim 11, wherein the heavy chain constant comprises an amino acid sequence set forth in SEQ ID NO: 49, 51, or 52.

14. The binding agent of claim 1, wherein the light chain variable region further comprises a light chain constant region.

15. The binding agent of claim 14, wherein the light chain constant region is of the kappa isotype.

16-17. (canceled)

18. The binding agent of claim 1, wherein the binding agent comprises
(a) a heavy chain and a light chain comprising the amino acid sequences set forth in SEQ ID NOs: 53 and 55, respectively, or
(b) a heavy chain and a light chain comprising the amino acid sequences set forth in SEQ ID NOs: 54 and 55, respectively.

19. (canceled)

20. The binding agent of claim 1, wherein the binding agent is mono-specific and/or bivalent.

21-22. (canceled)

23. A nucleic acid encoding the binding agent of claim 1.

24. A vector comprising the nucleic acid of claim 23.

25. A cell line comprising the nucleic acid of claim 23.

26. A conjugate comprising:
the binding agent of claim 1,
at least one linker attached to the binding agent;
at least one drug unit, wherein each drug unit is attached to a linker, wherein the linker optionally comprises at least one polar group.

27. The conjugate of claim 26, wherein the linker is derived from a linker compound, or a stereoisomer or salt thereof, and the linker compound comprises:
the linker unit;
a stretcher group connected to the linker unit,
an optional amino acid unit; and
the at least one polar group; wherein:
the stretcher group has an attachment site to the binding agent and an attachment site to the amino acid unit (when present) or the linker subunit;
the amino acid unit (when present) has an attachment site to the stretcher group and an attachment site to the linker unit; and
the linker unit has an attachment site to the amino acid unit (when present) or to the stretcher group and to the at least one drug unit.

28. The conjugate of claim 26, wherein the drug unit is selected from a cytotoxic agent, an immune modulatory agent, a nucleic acid, a growth inhibitory agent, a PROTAC, a toxin, a radioactive isotope and a chelating ligand.

29-30. (canceled)

31. The conjugate of claim 26, selected from the following: 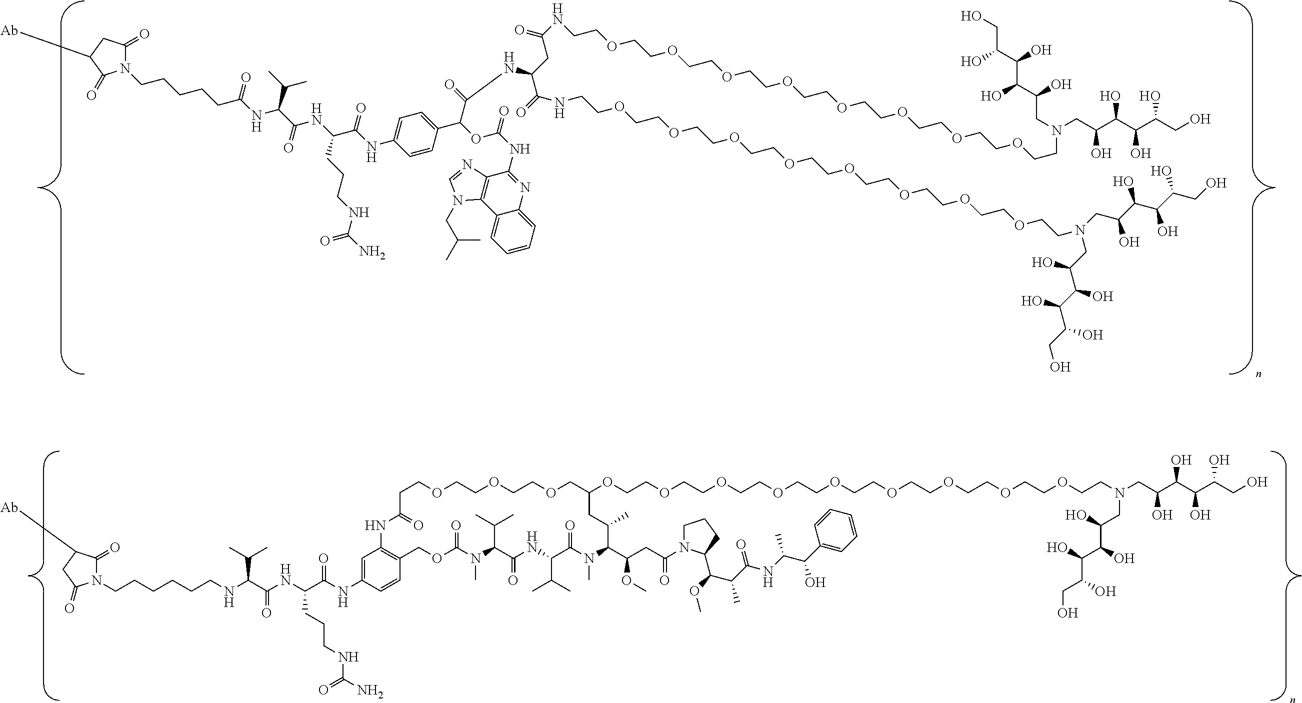 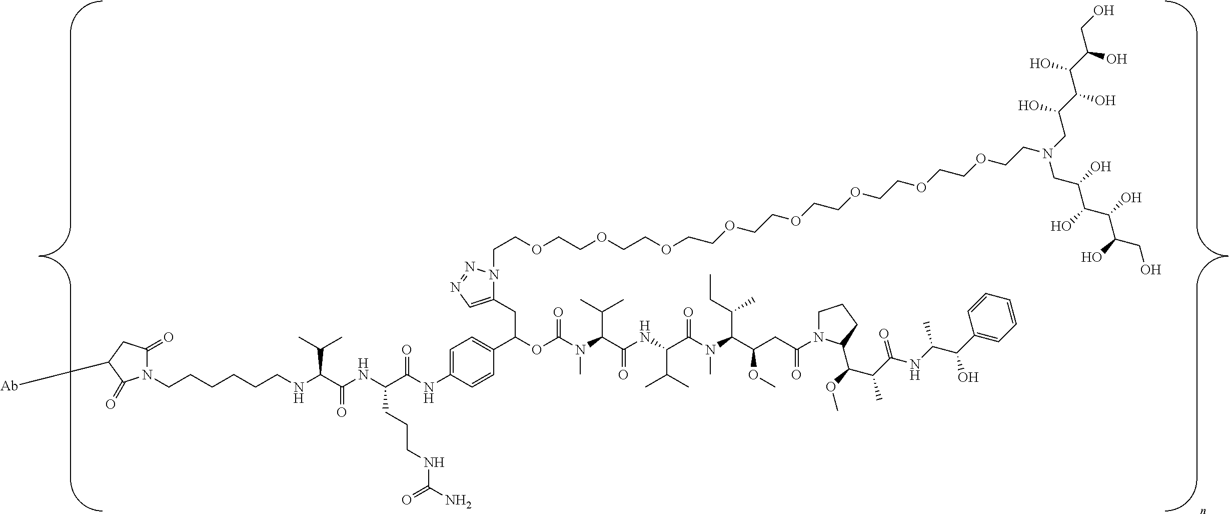 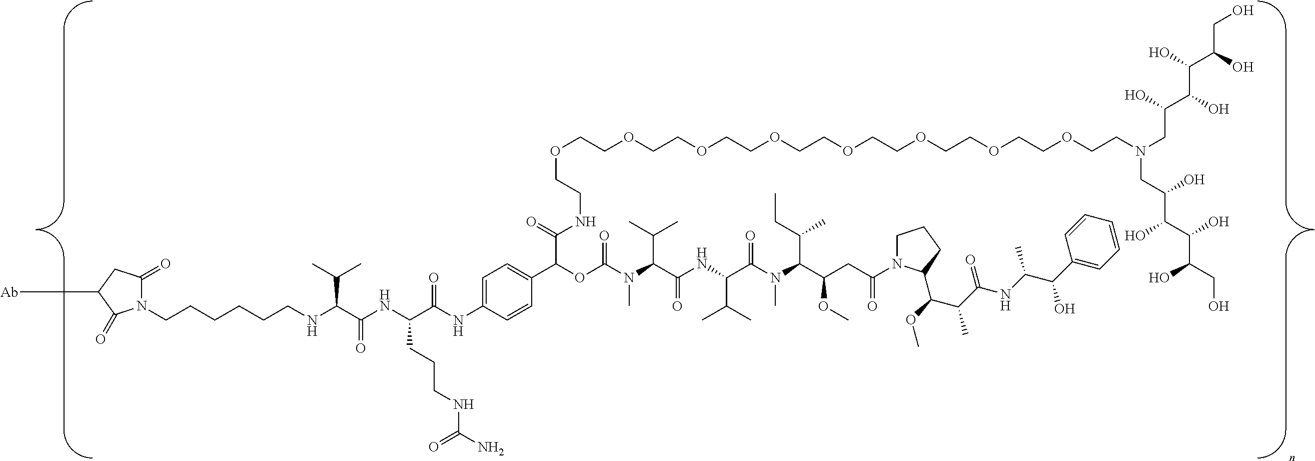 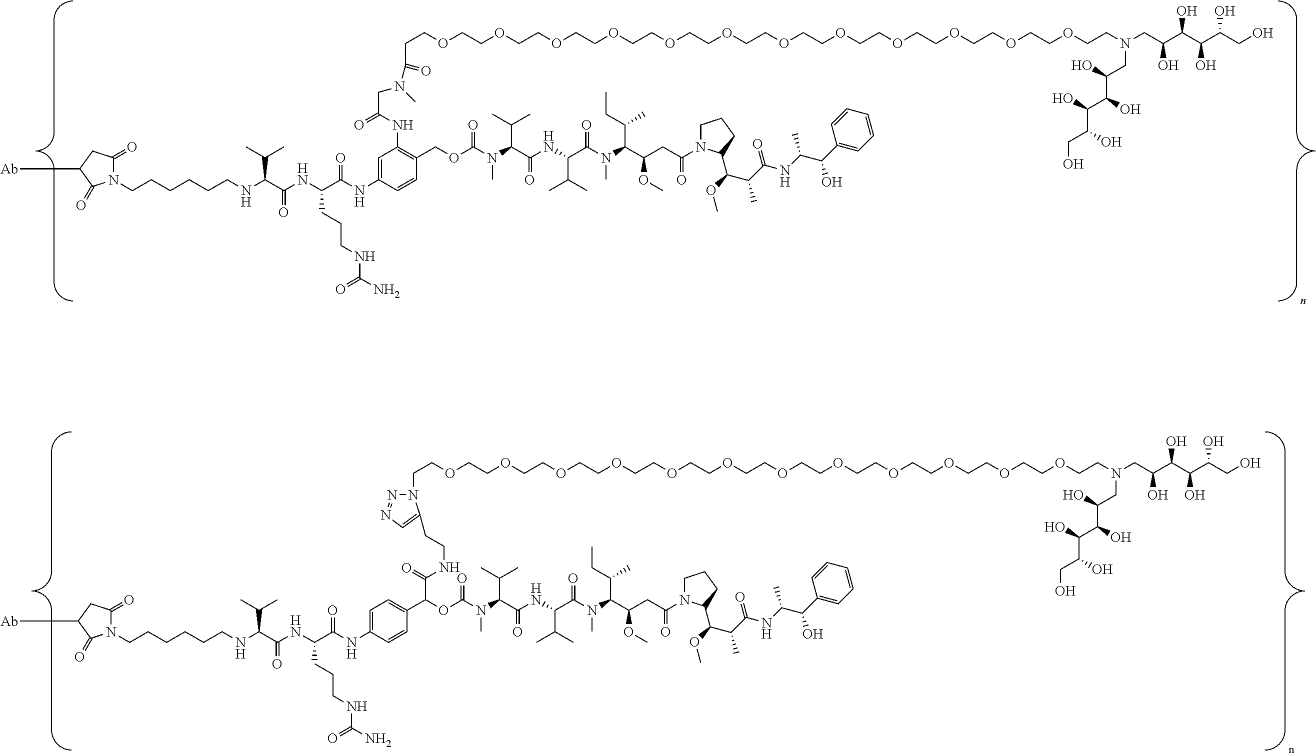 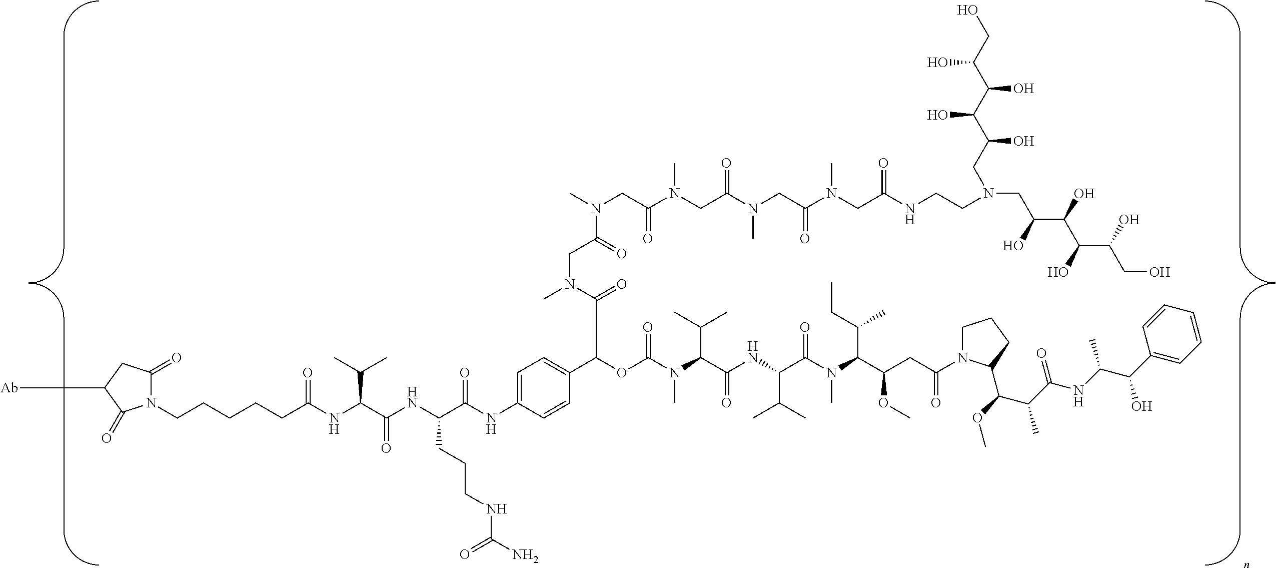 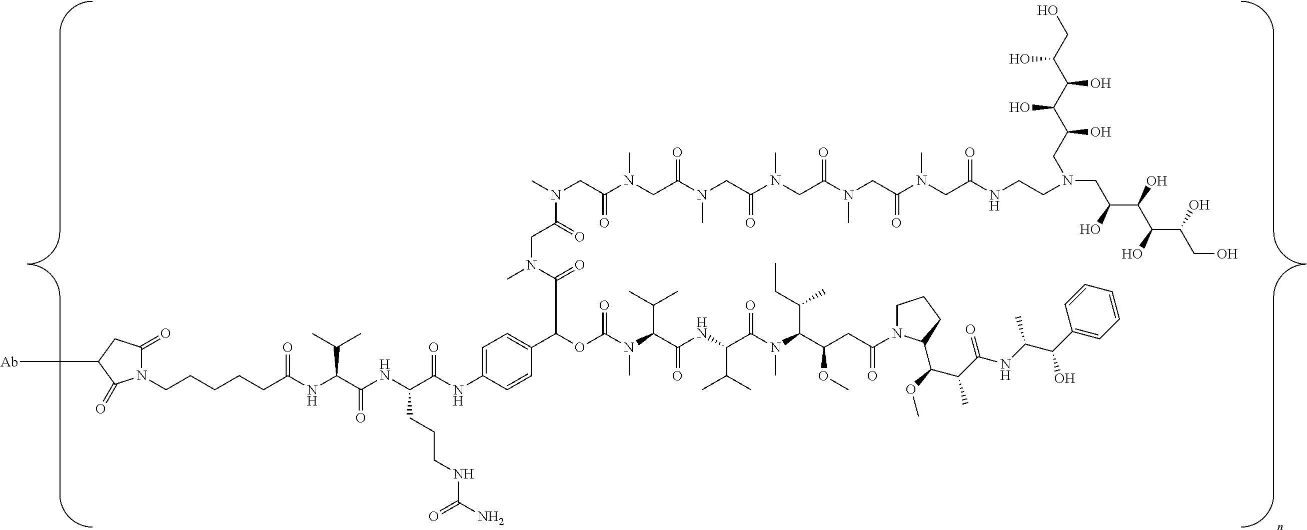 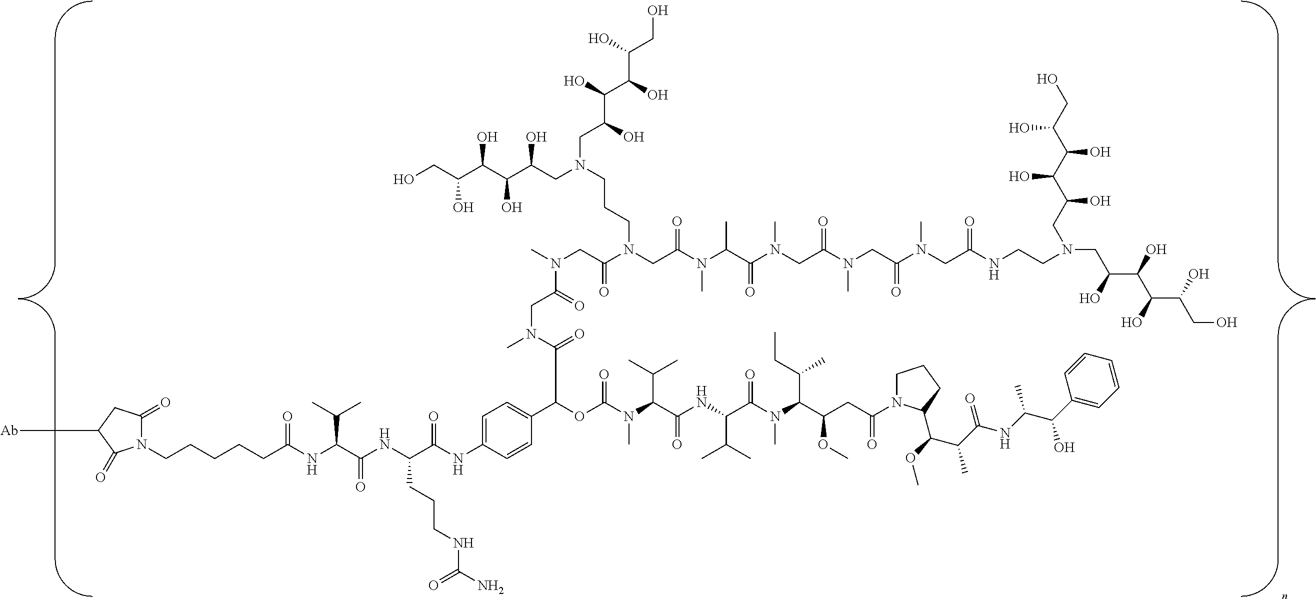 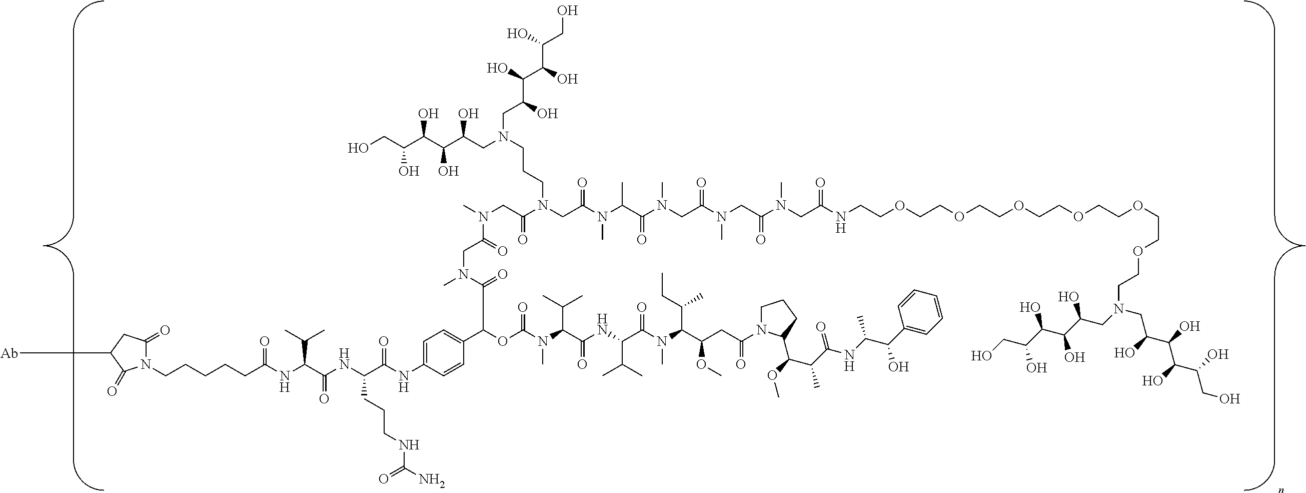 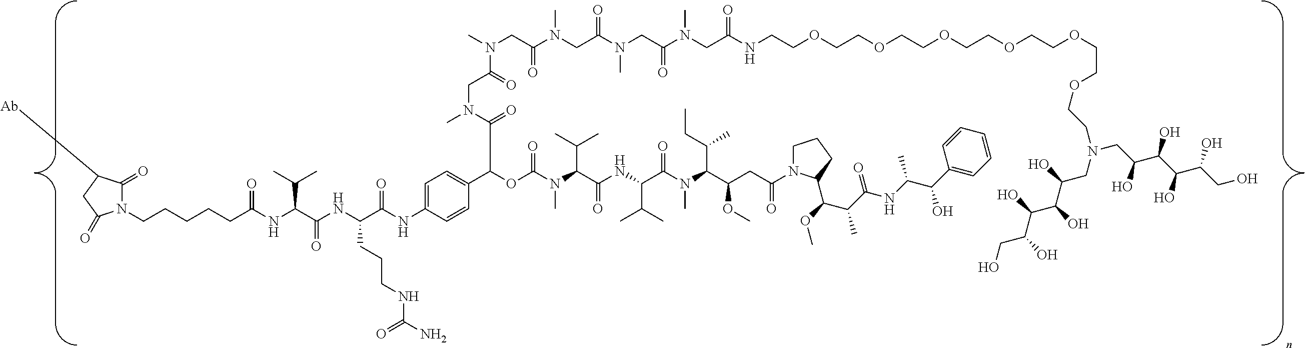 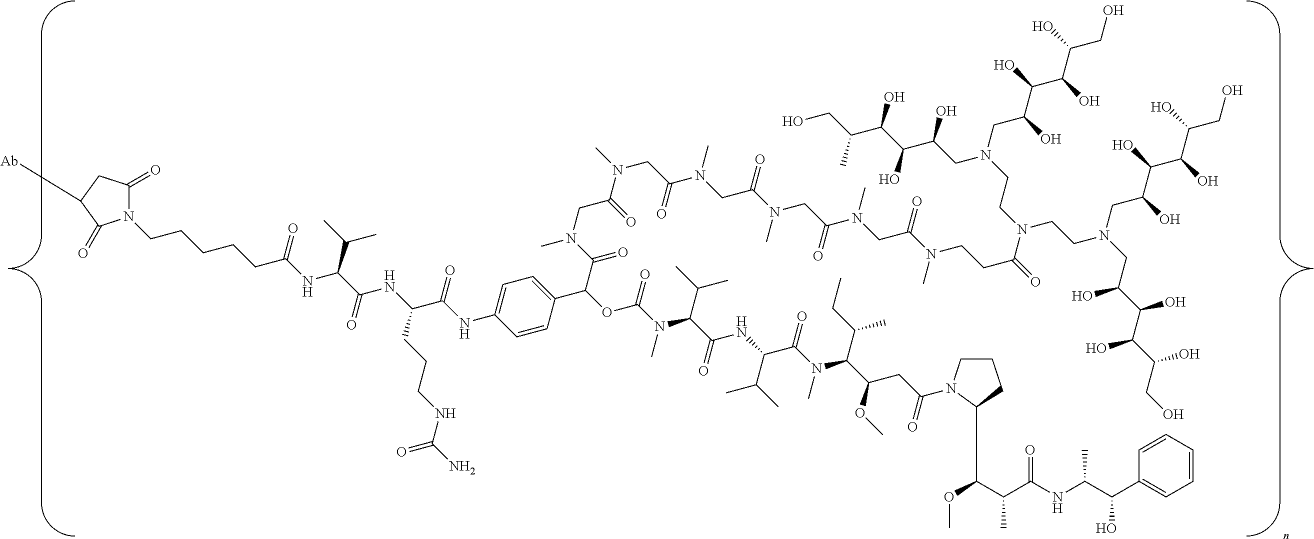 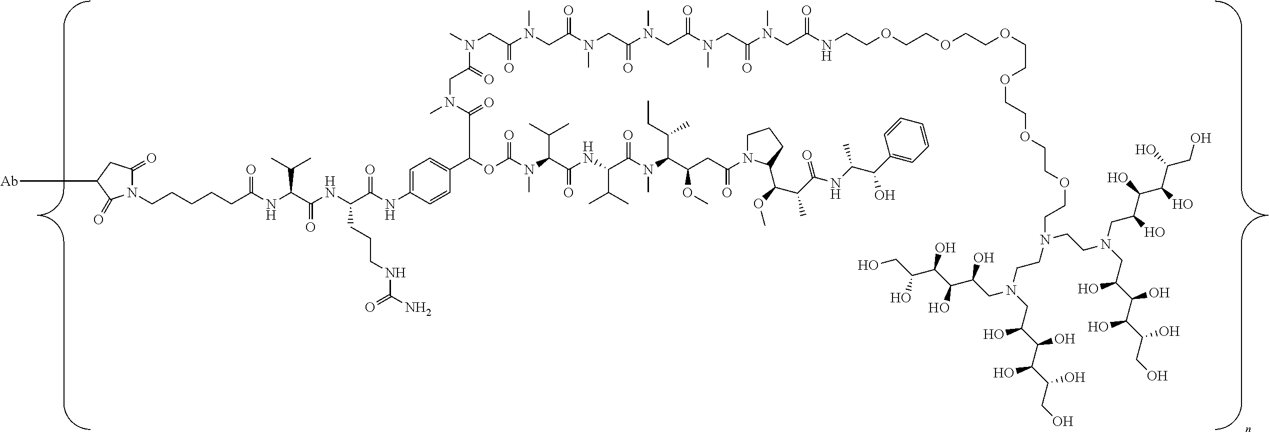 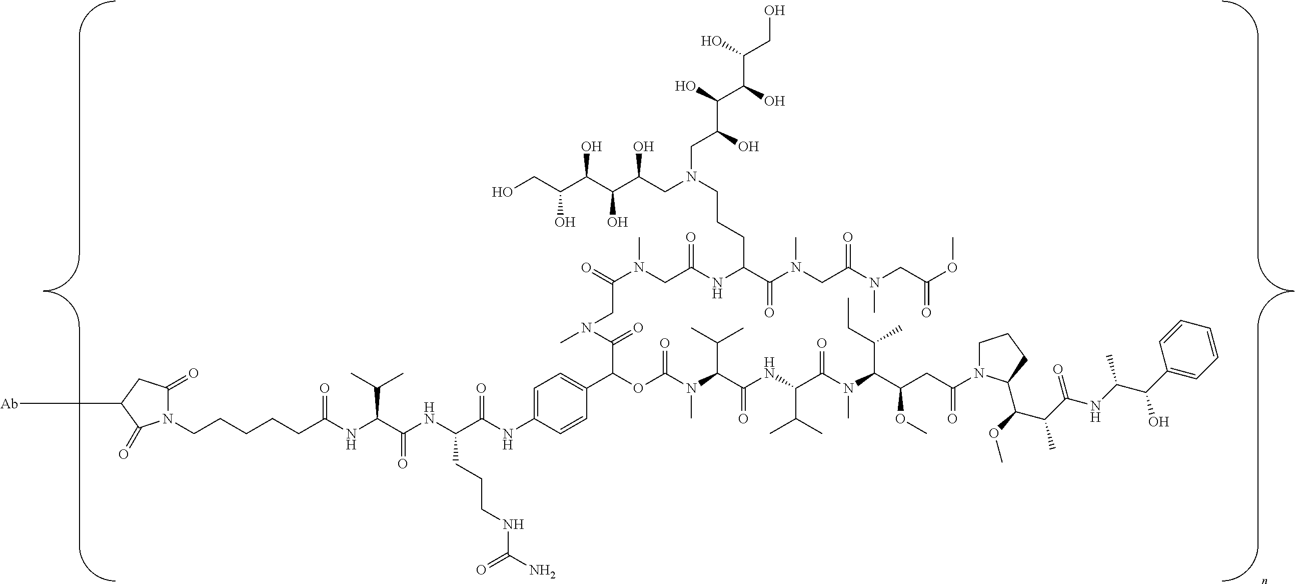 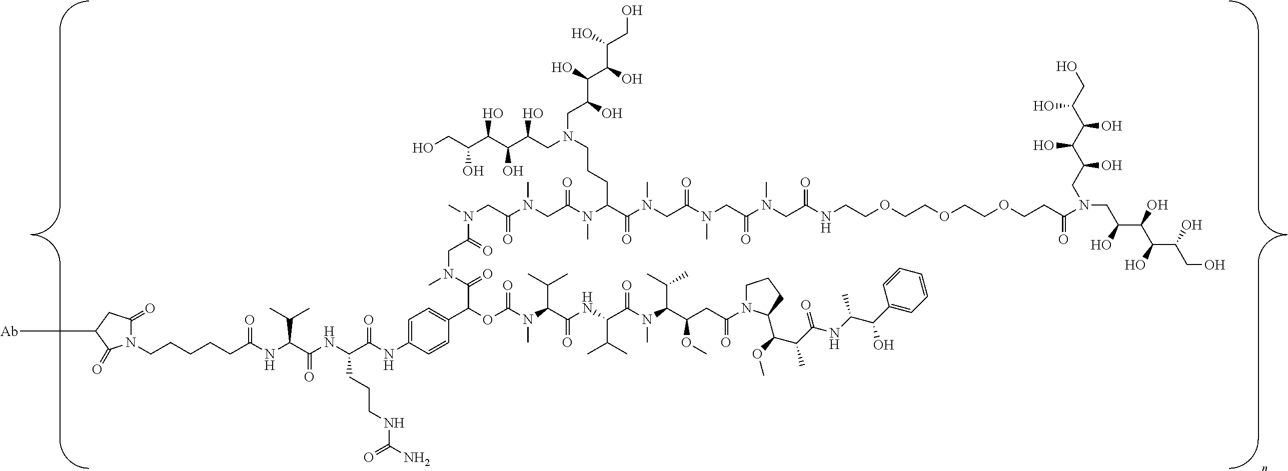 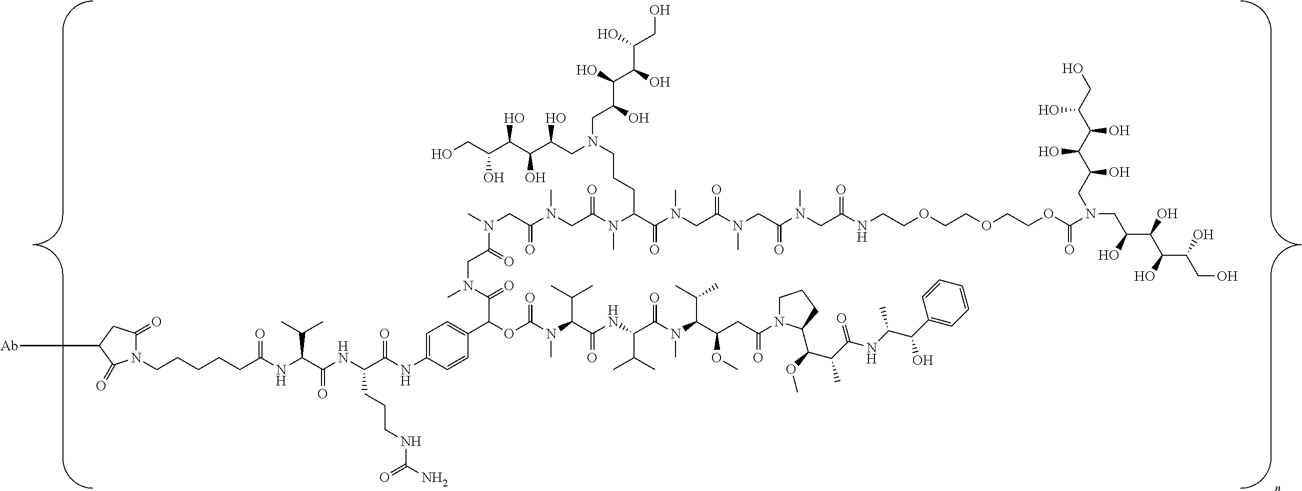 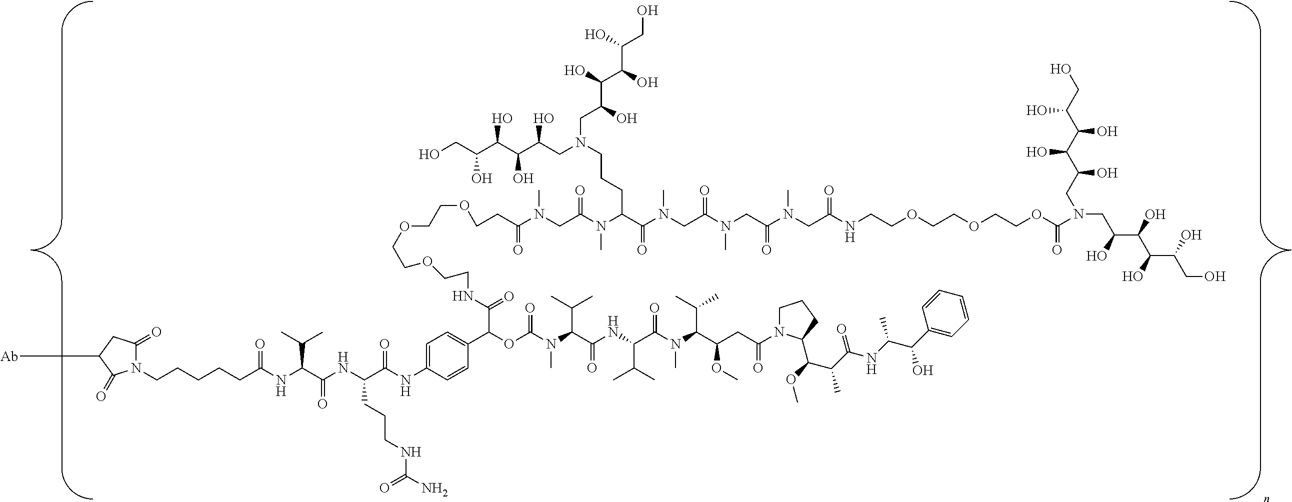 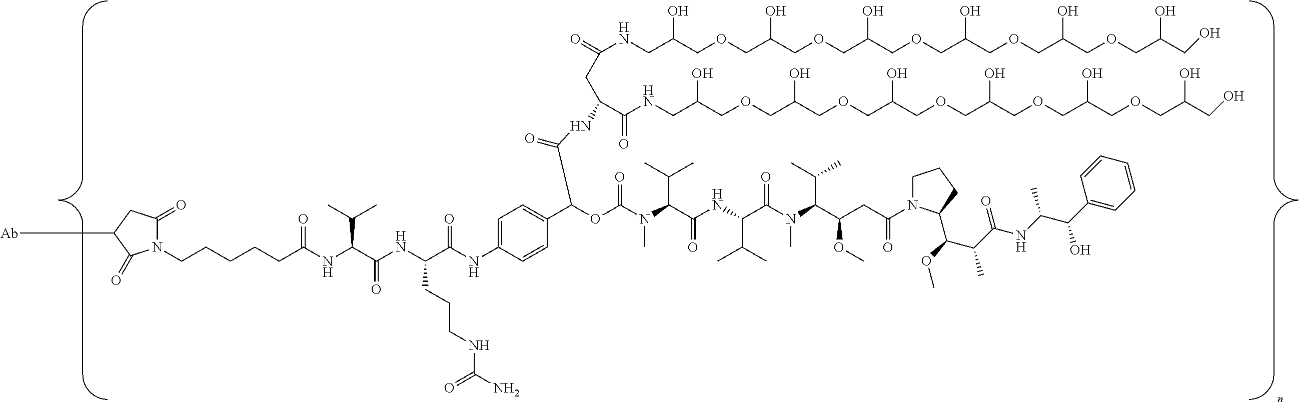 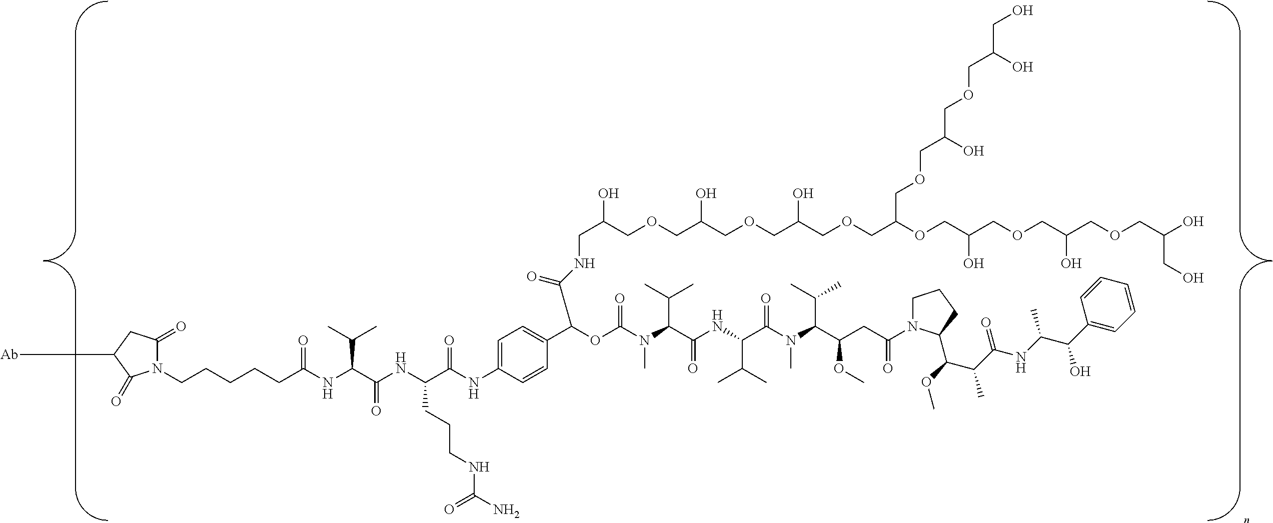 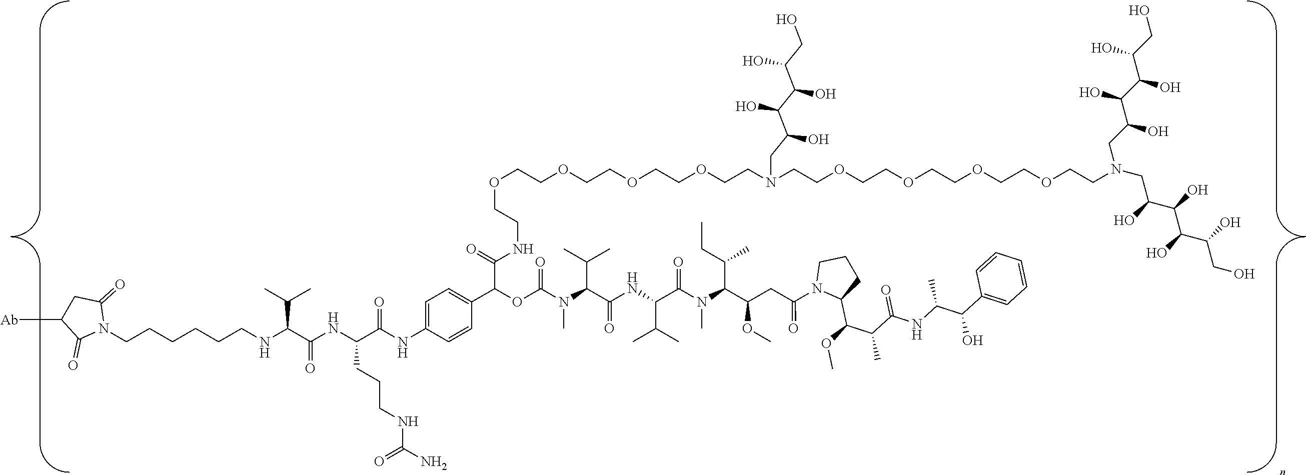 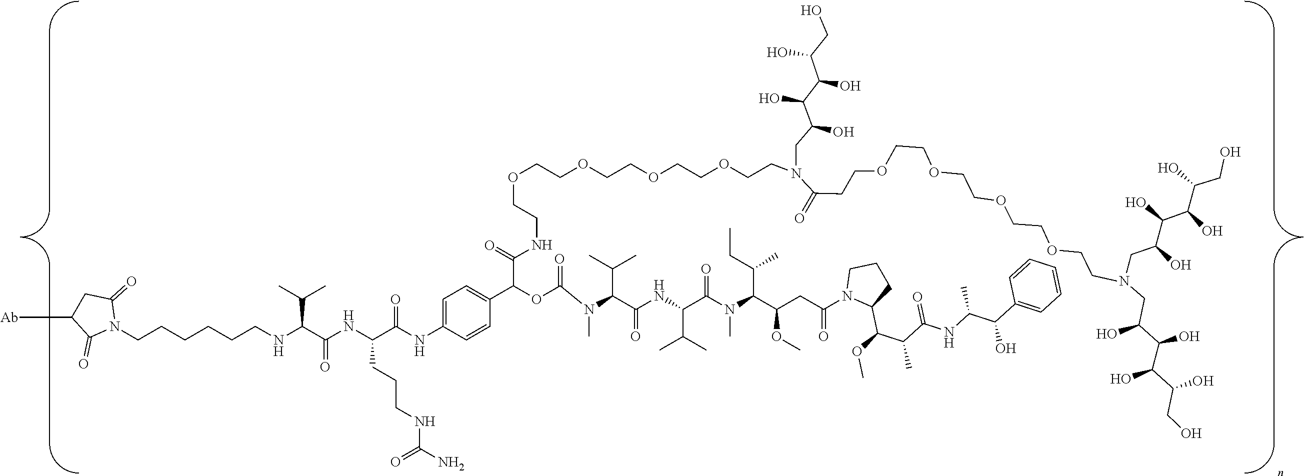 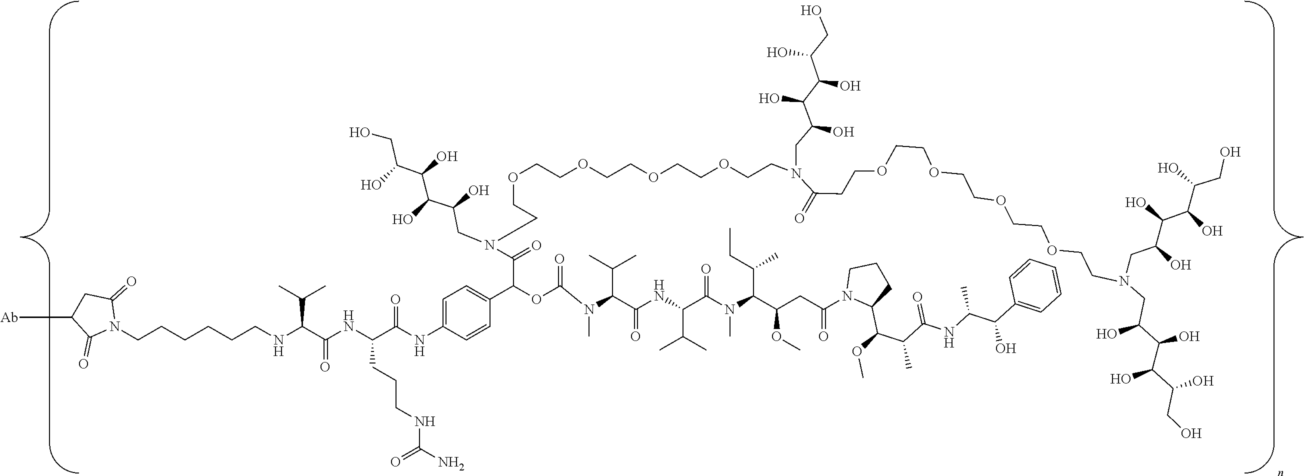 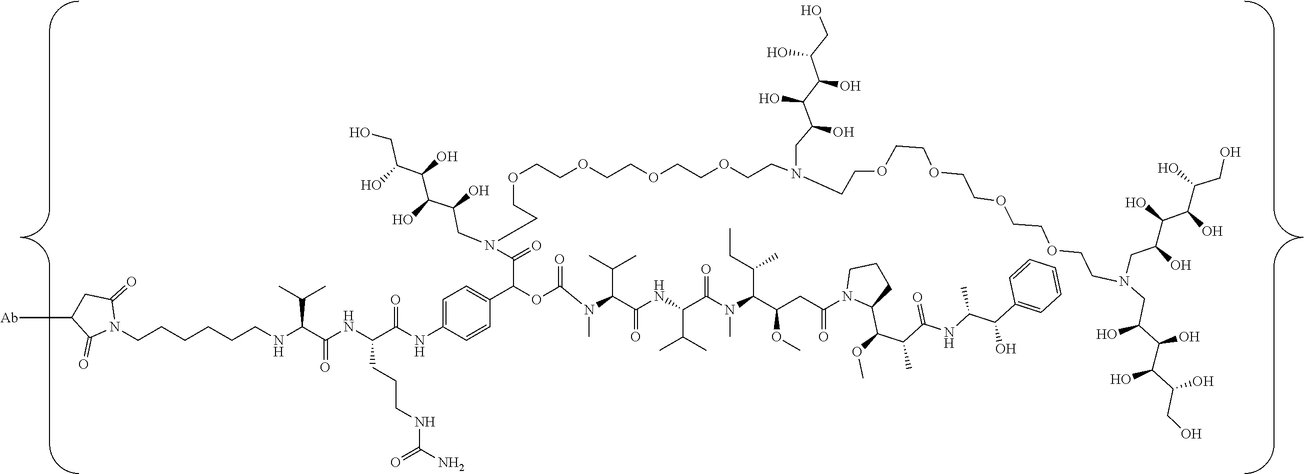 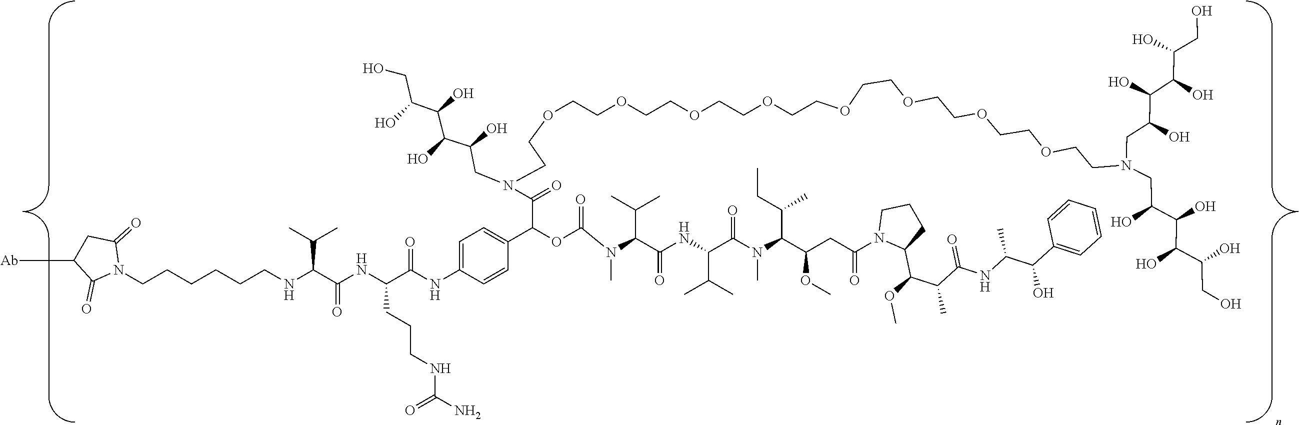 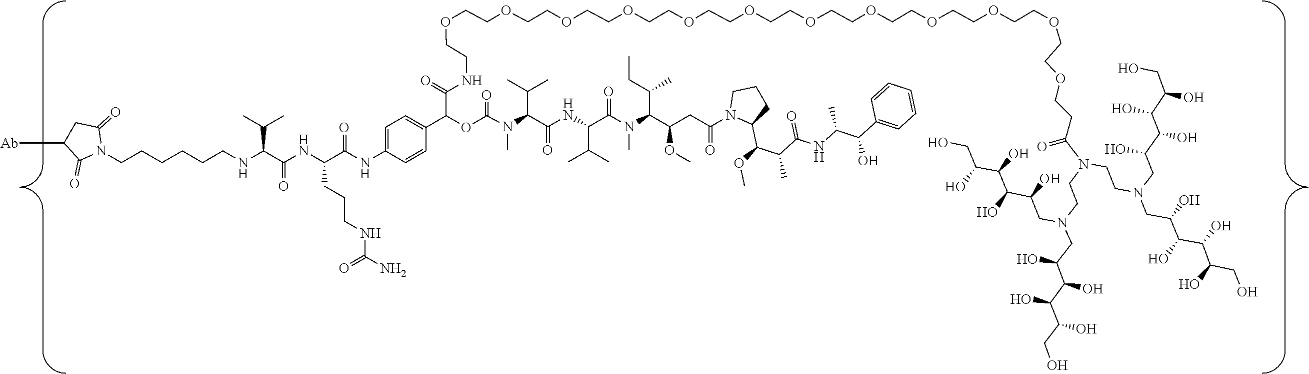 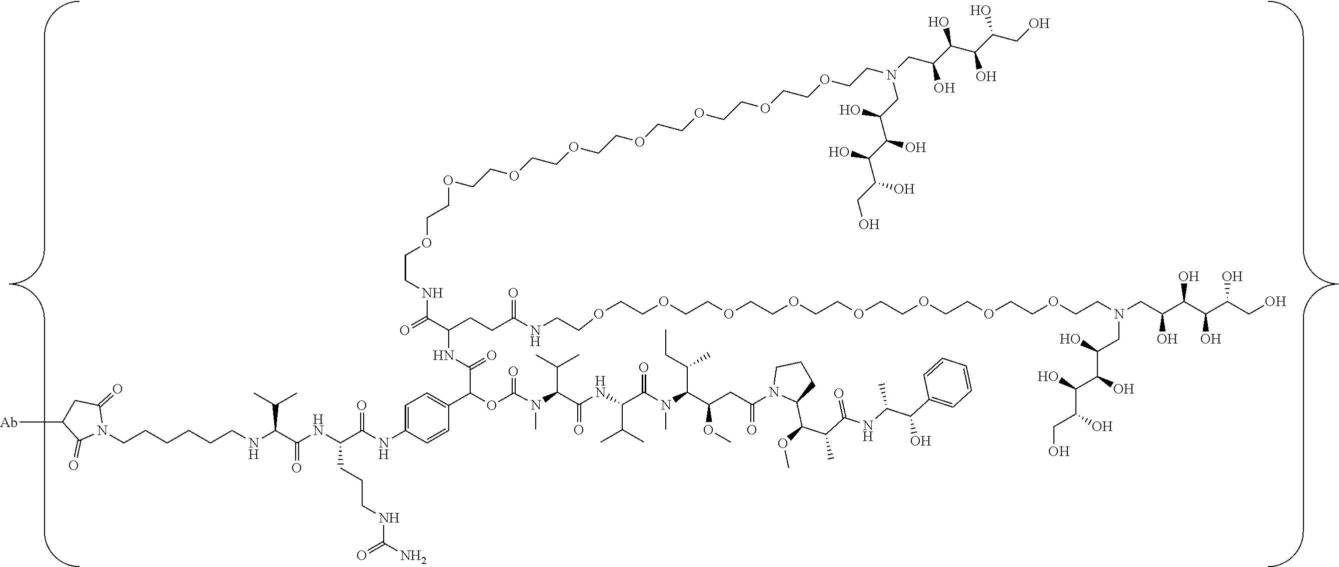 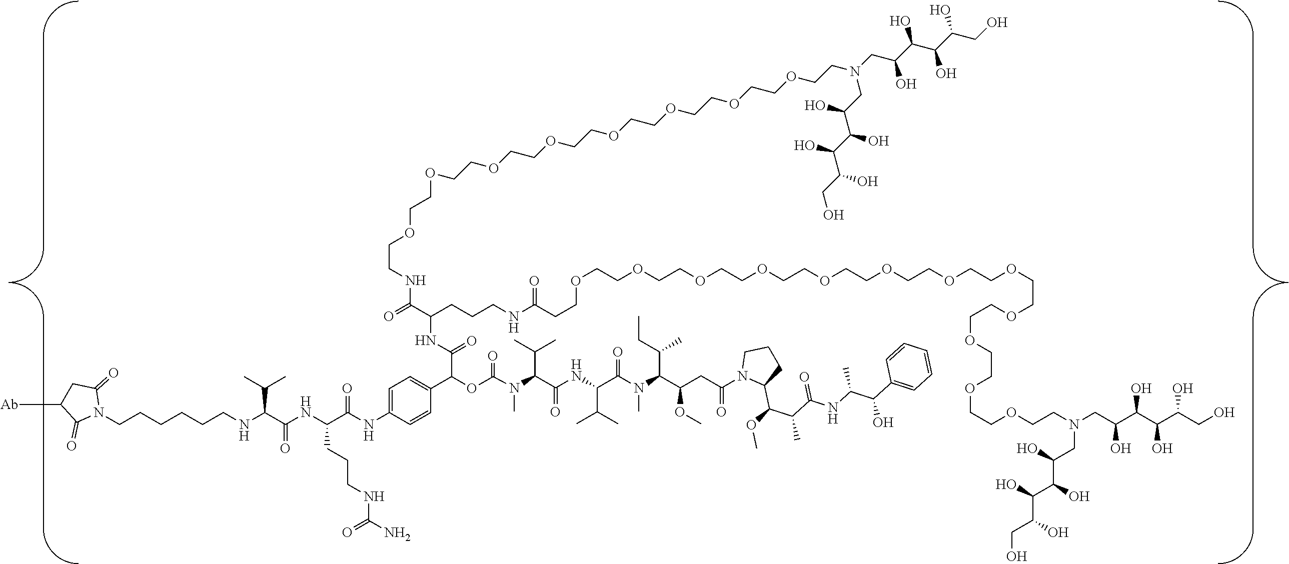 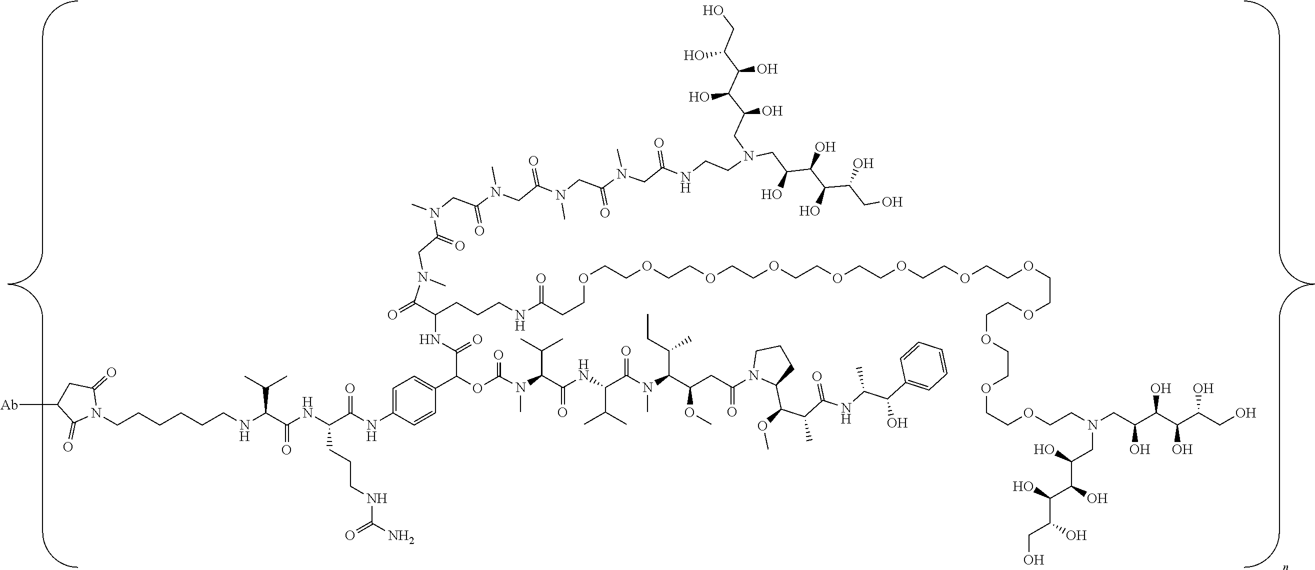 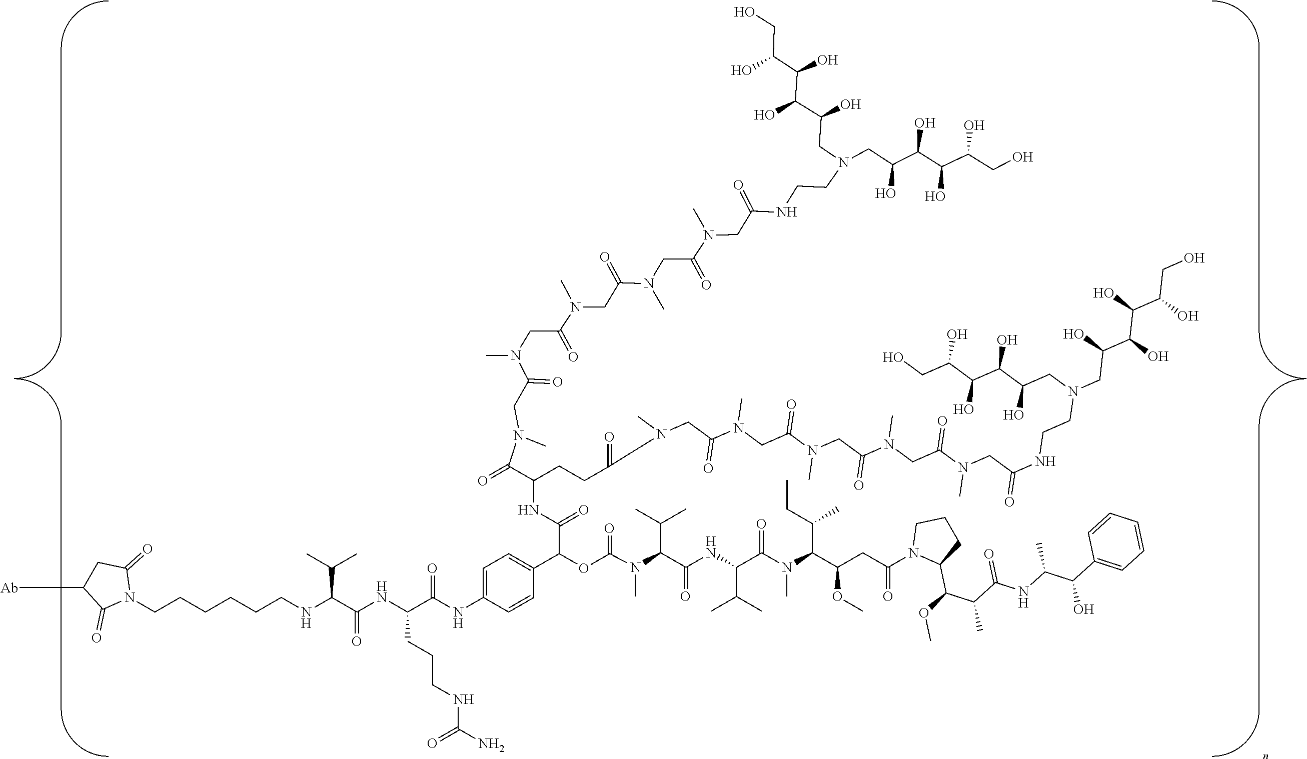 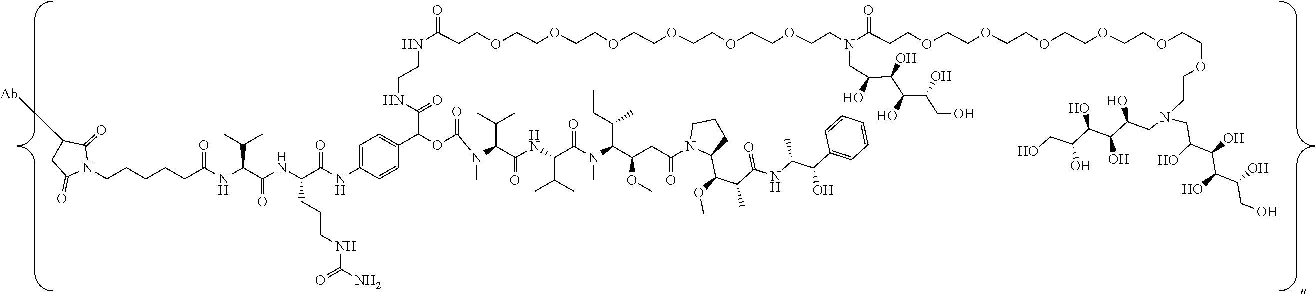 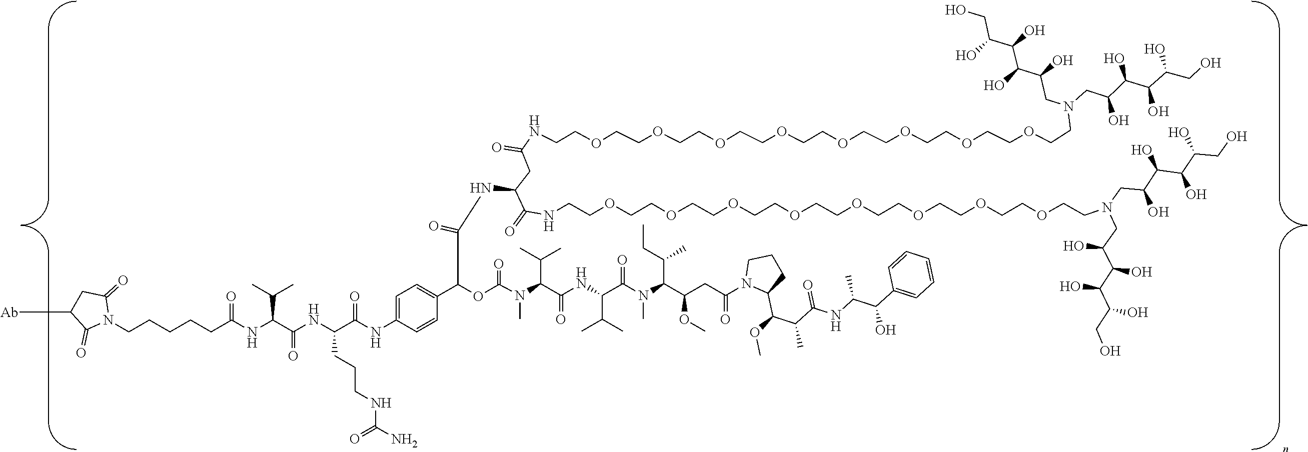 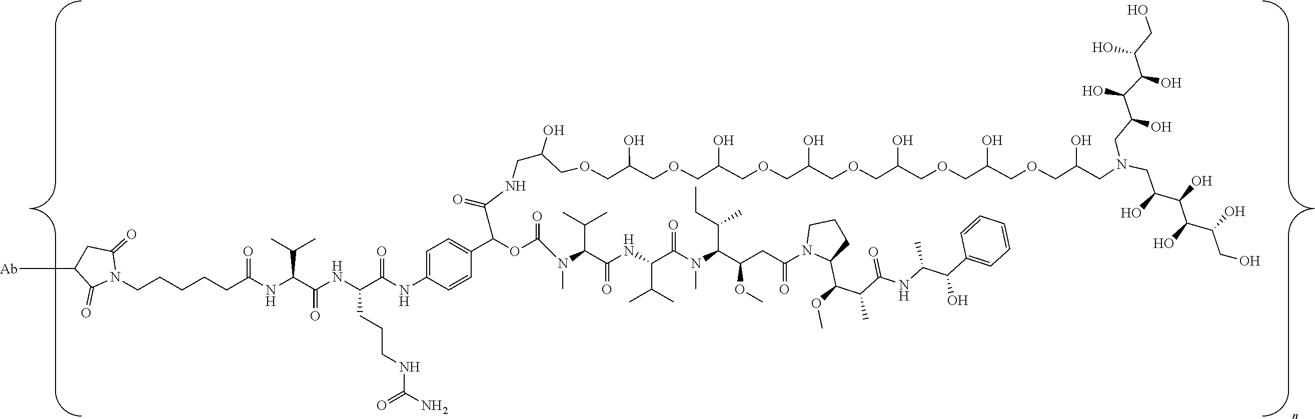 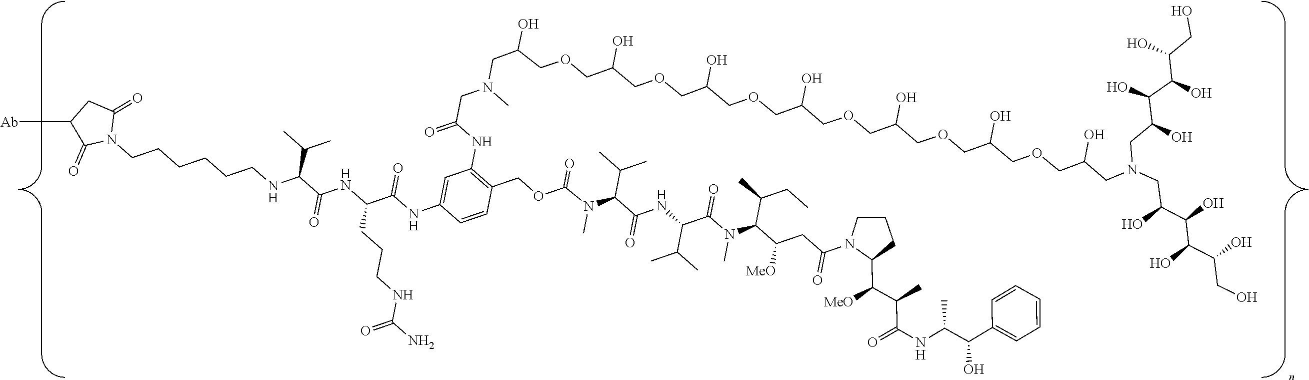 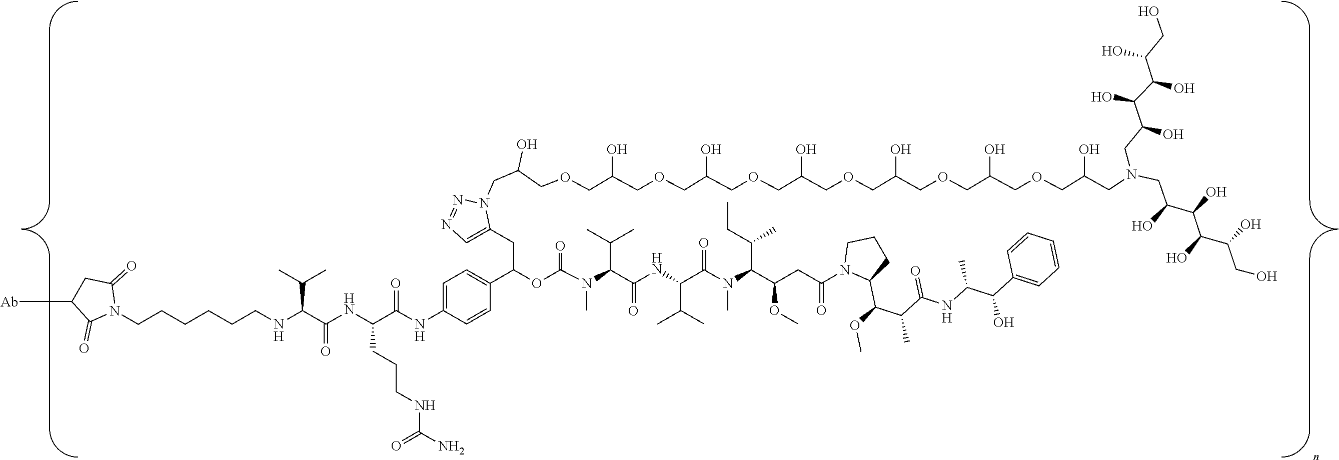 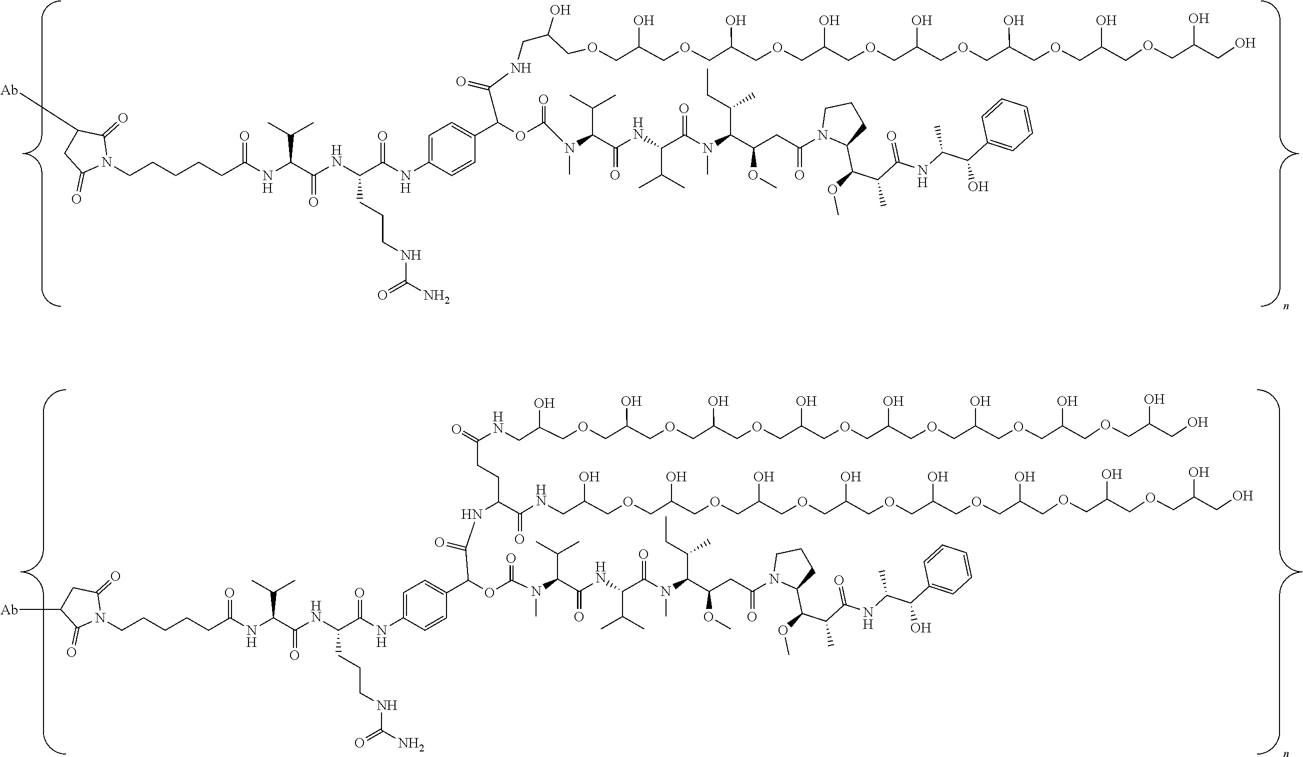 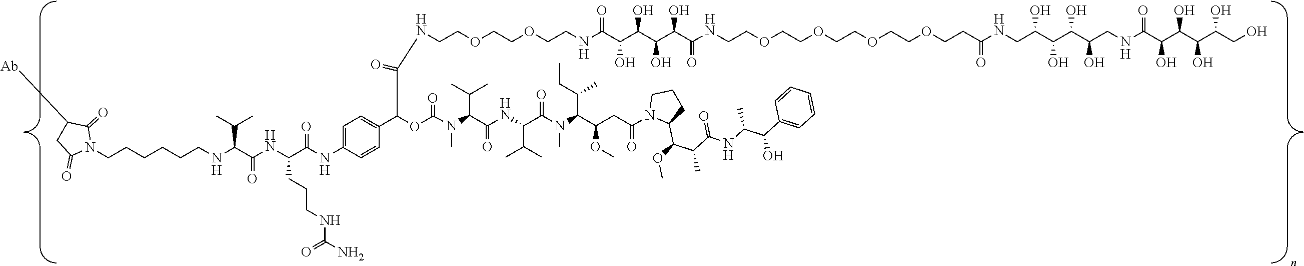 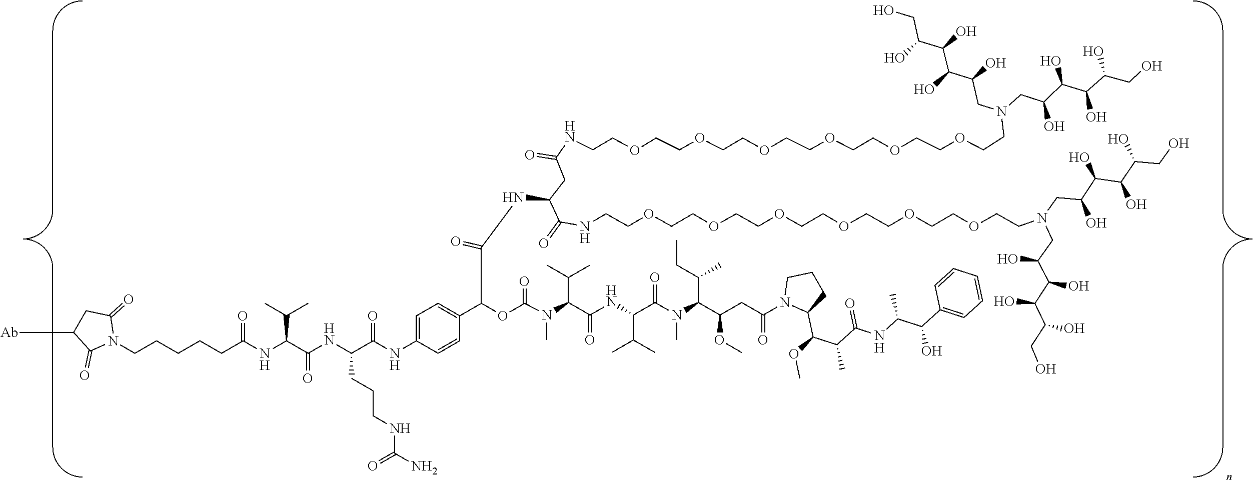 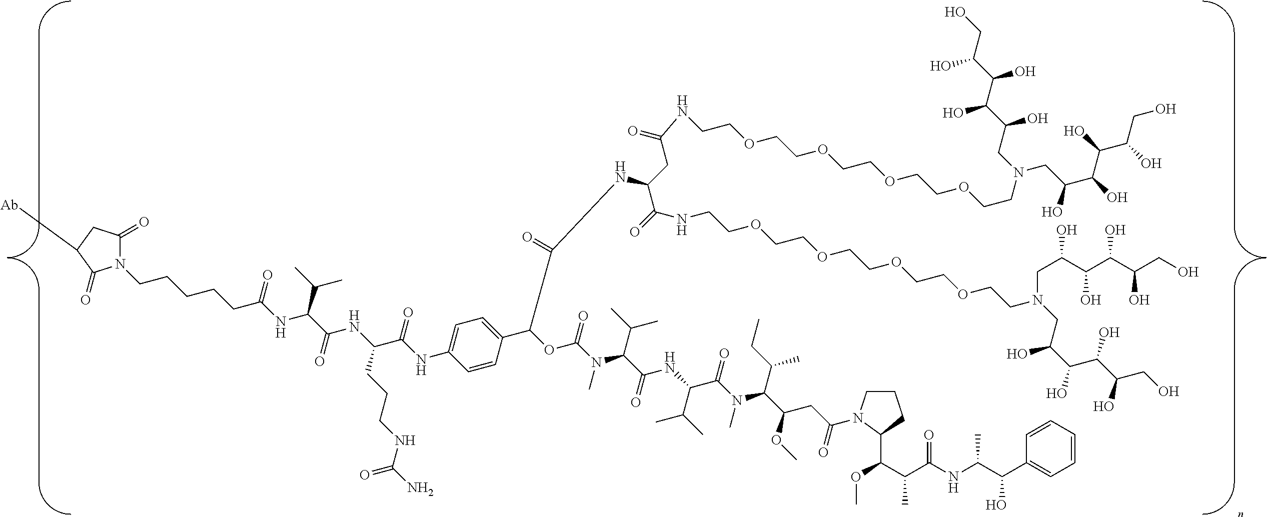 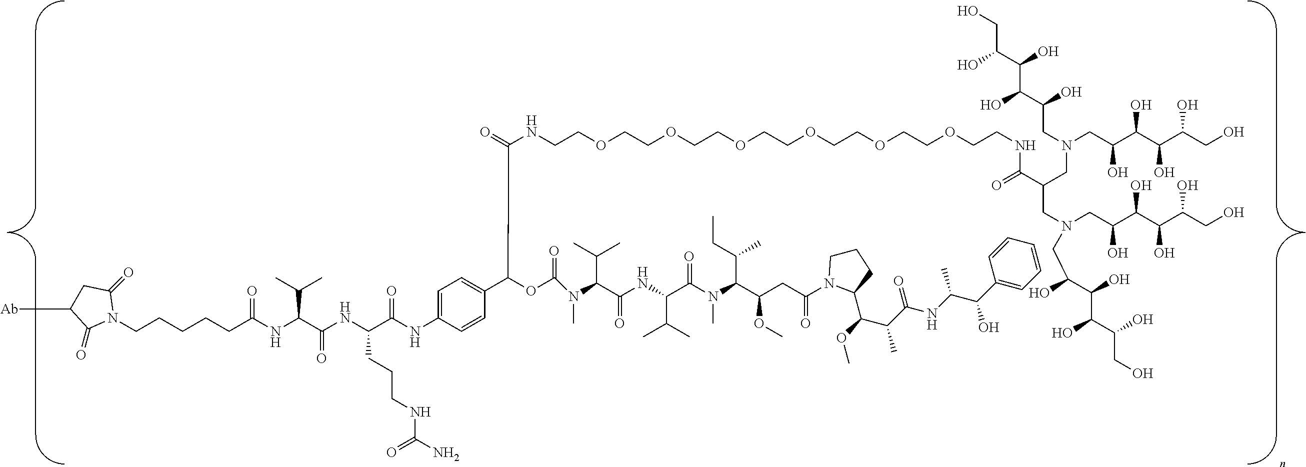 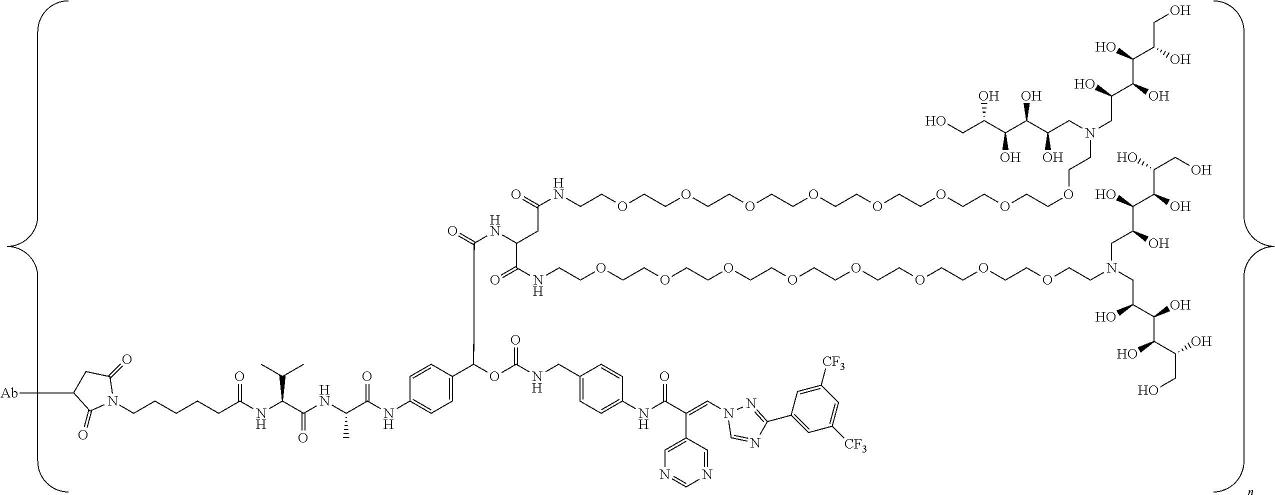 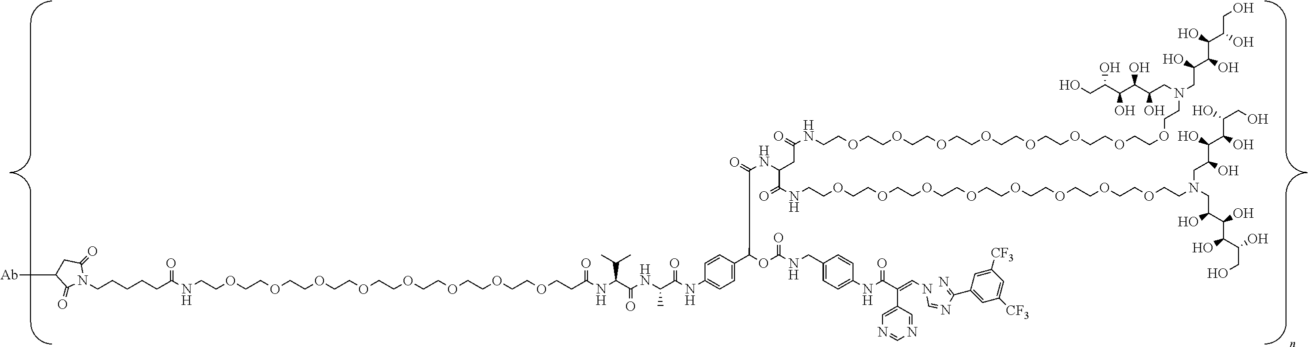 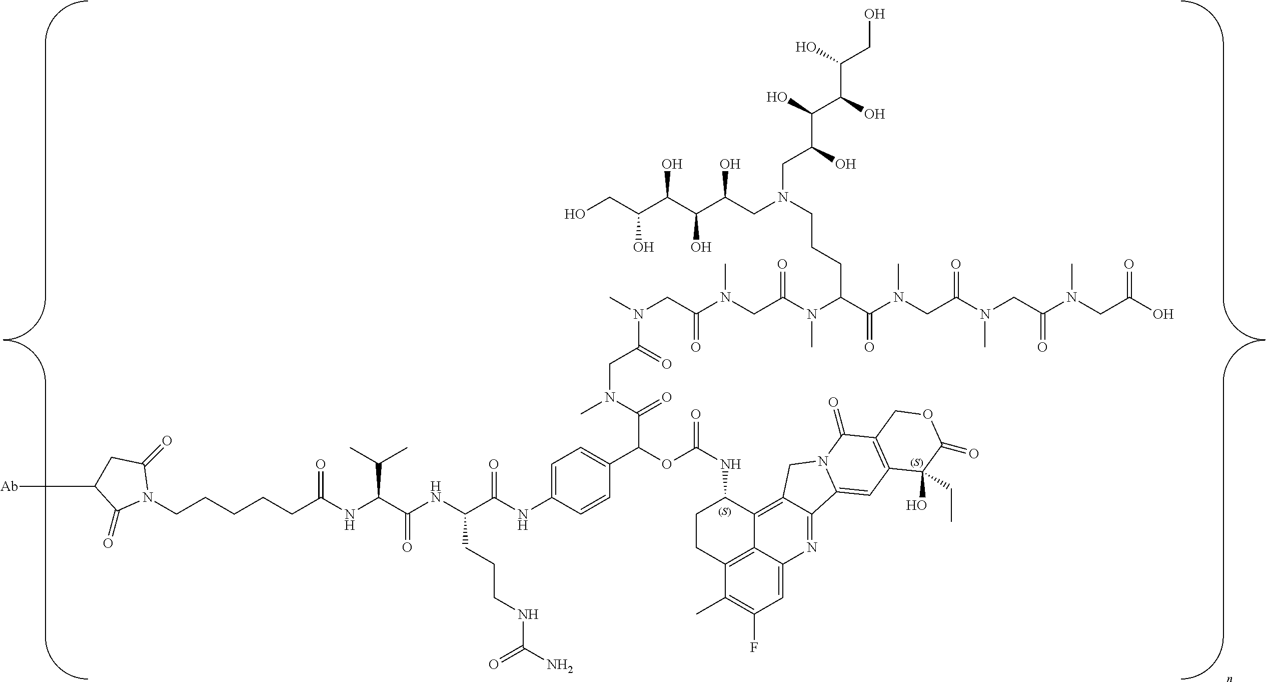 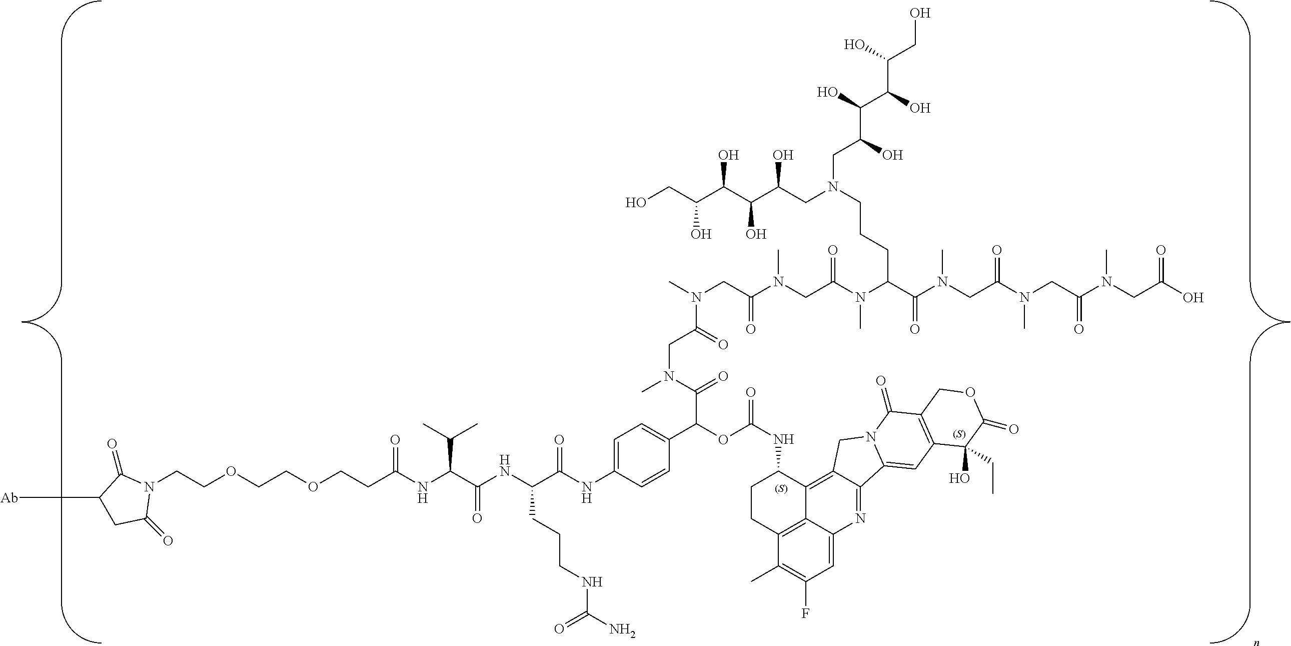 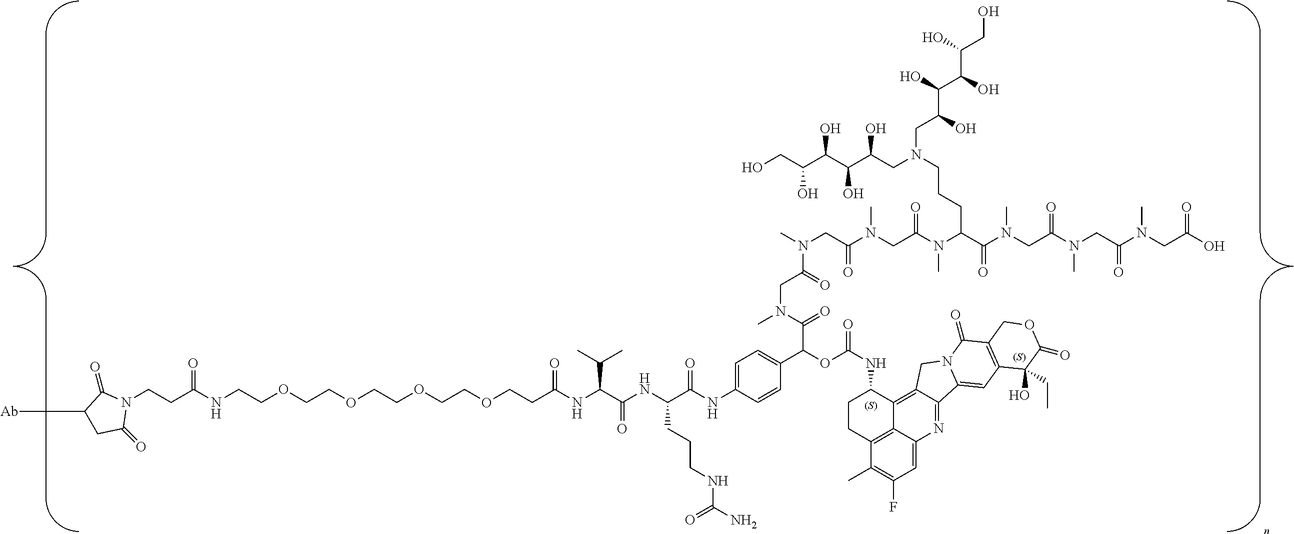 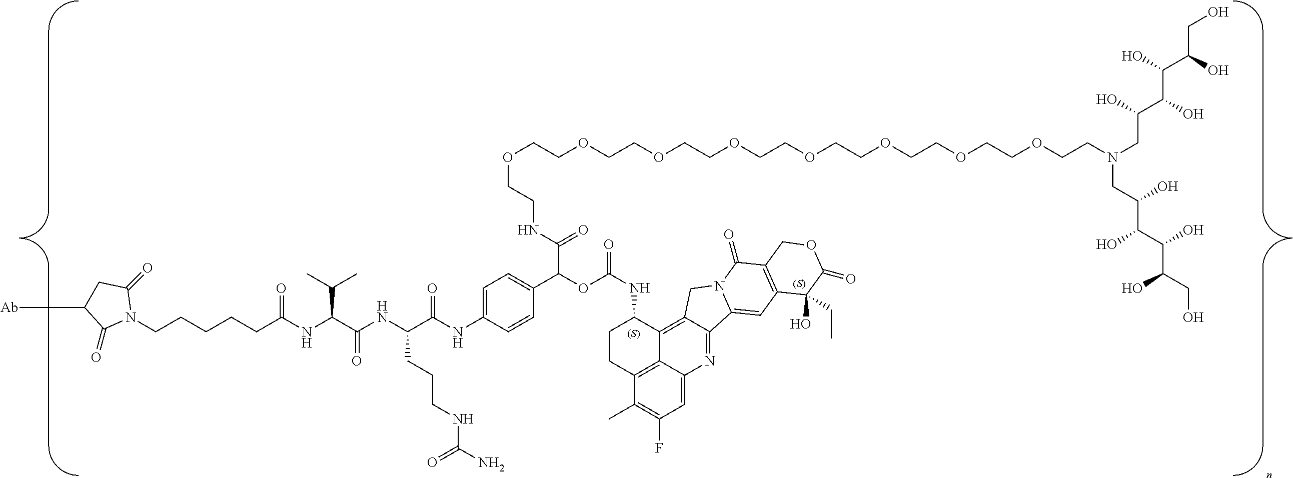 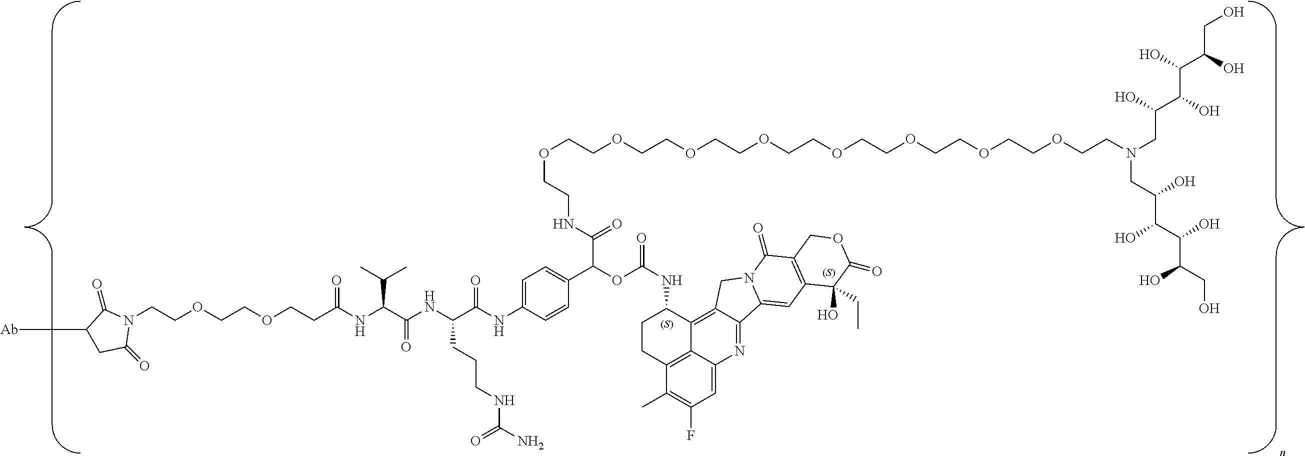 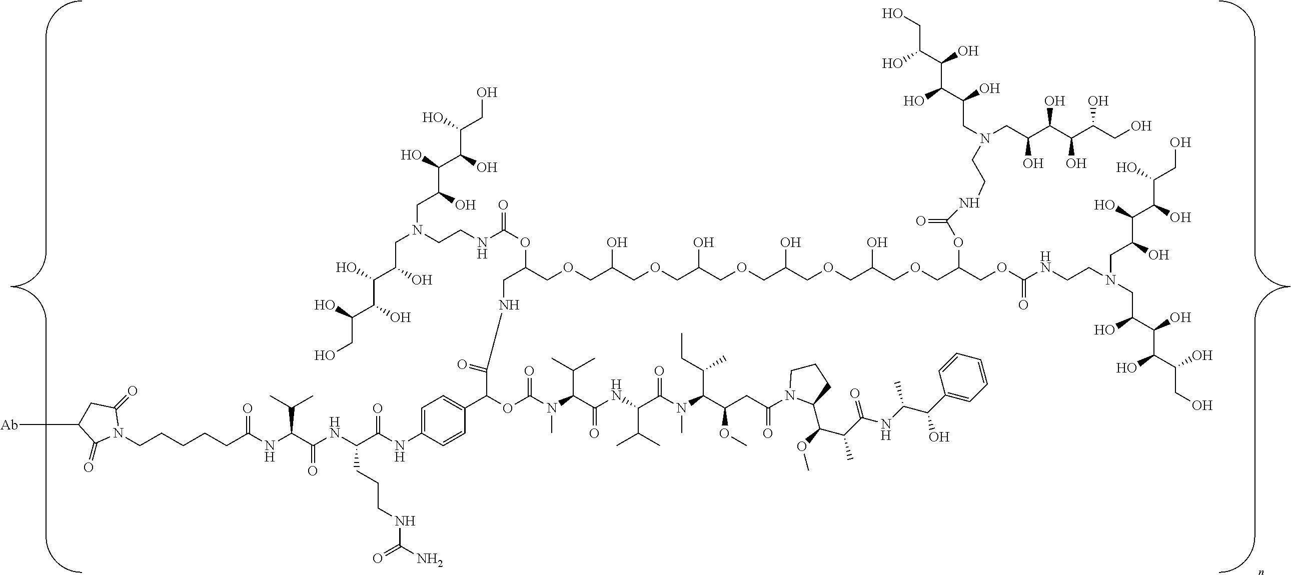 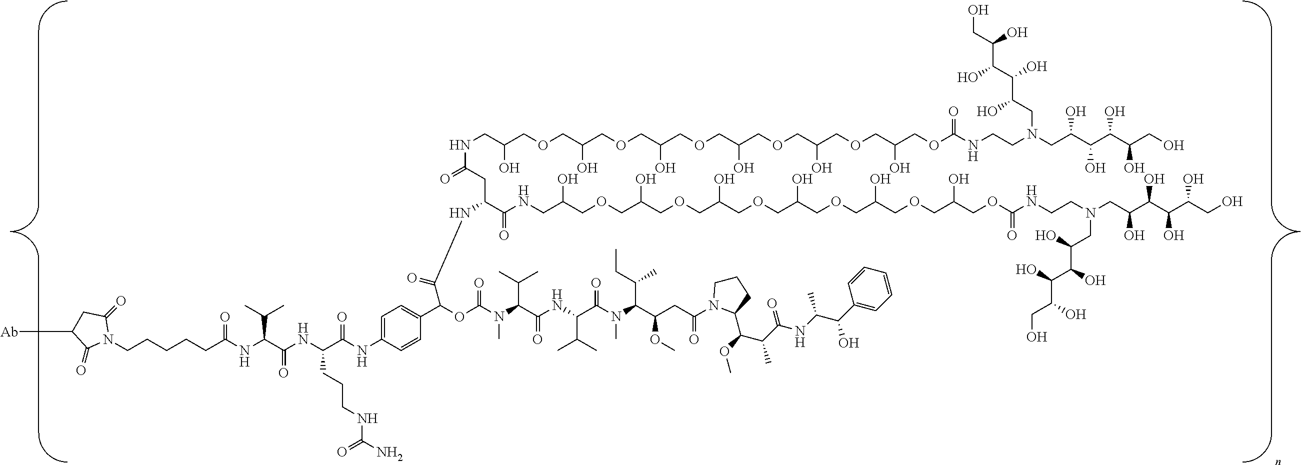 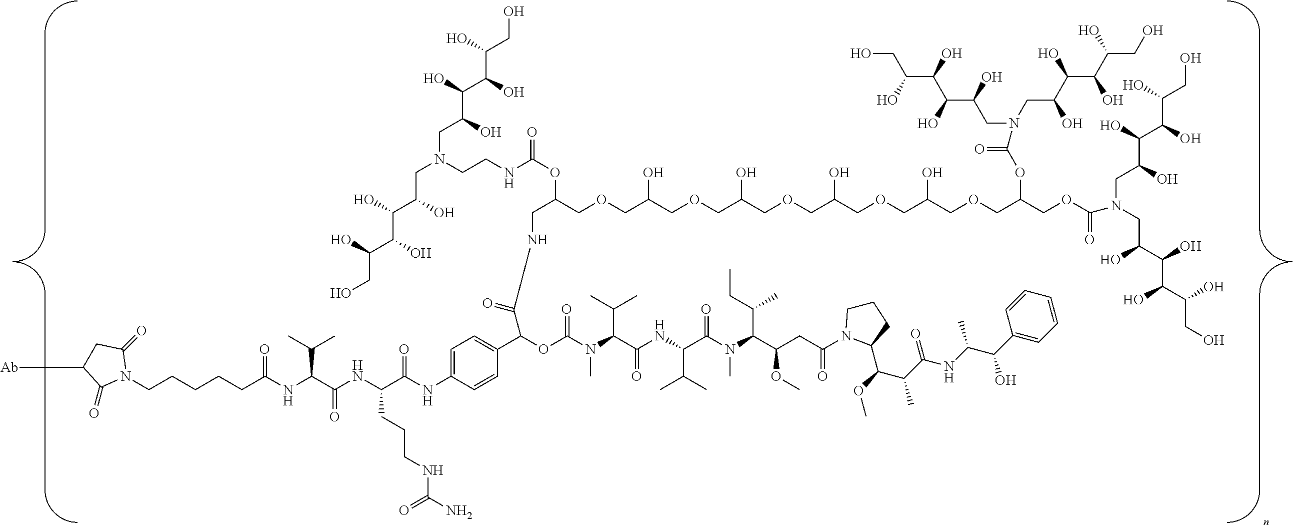 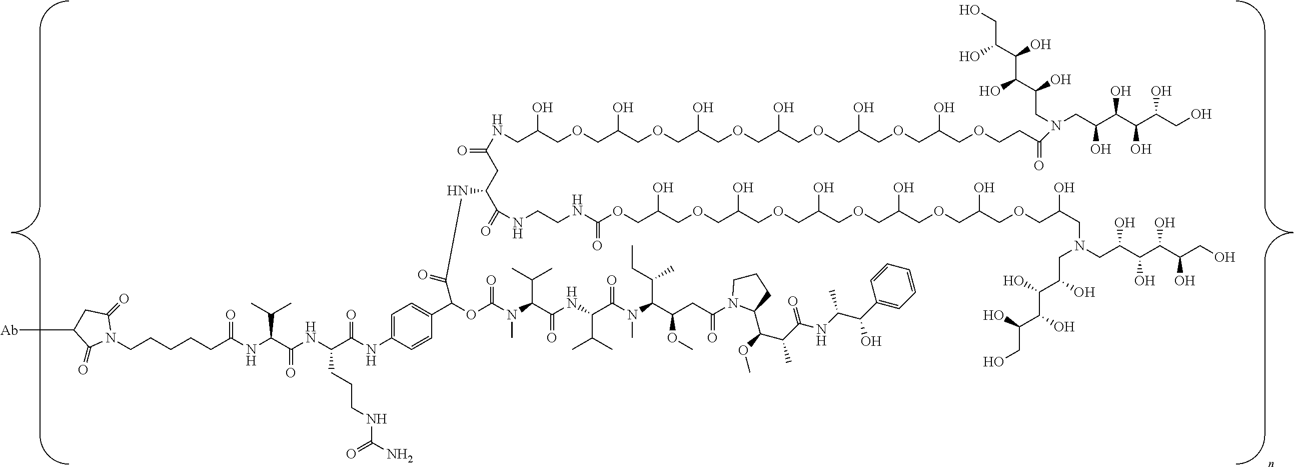 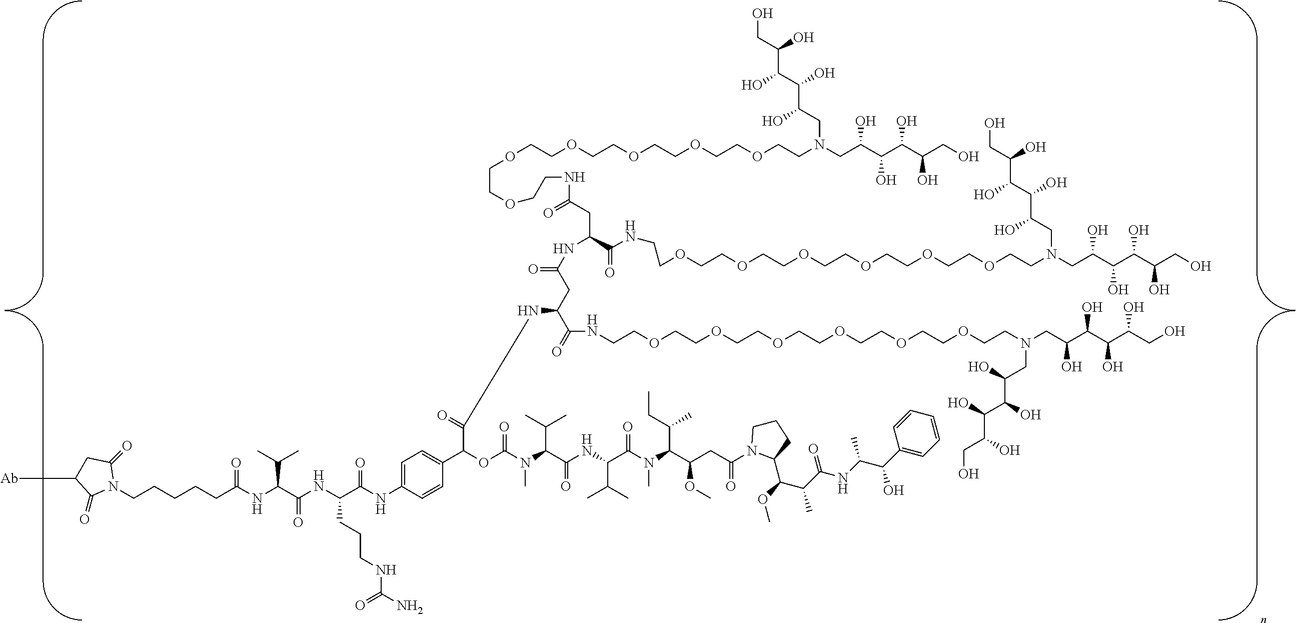 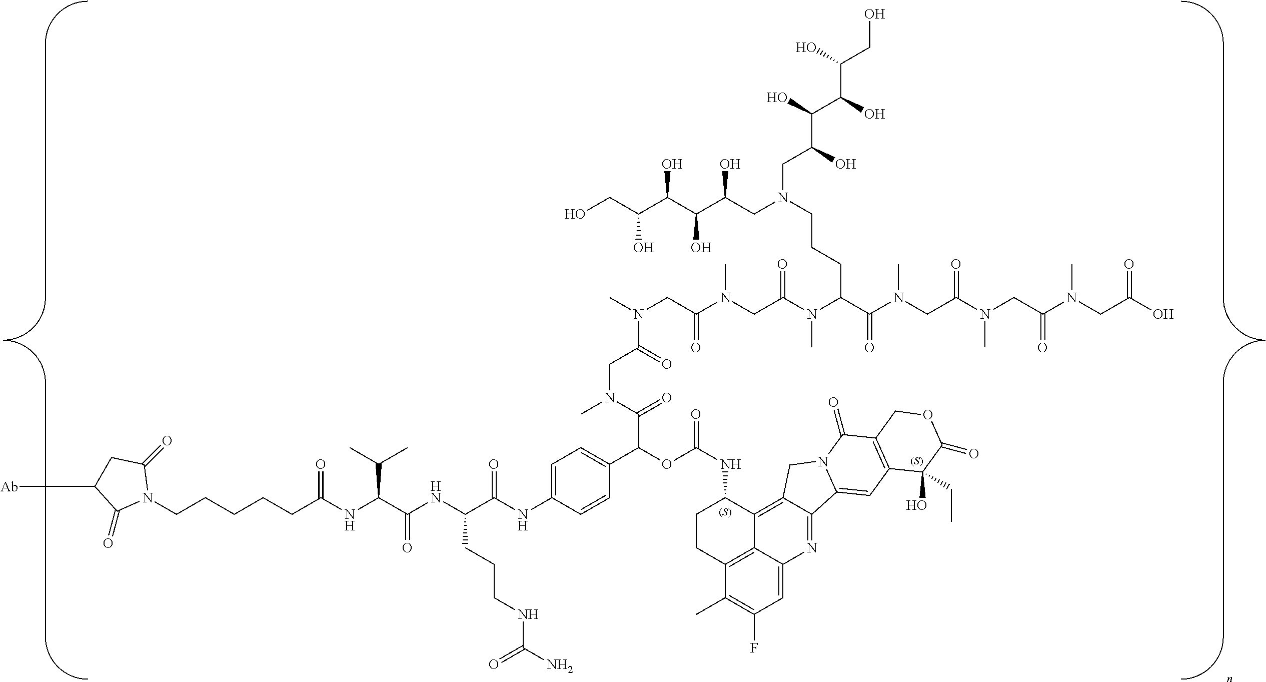 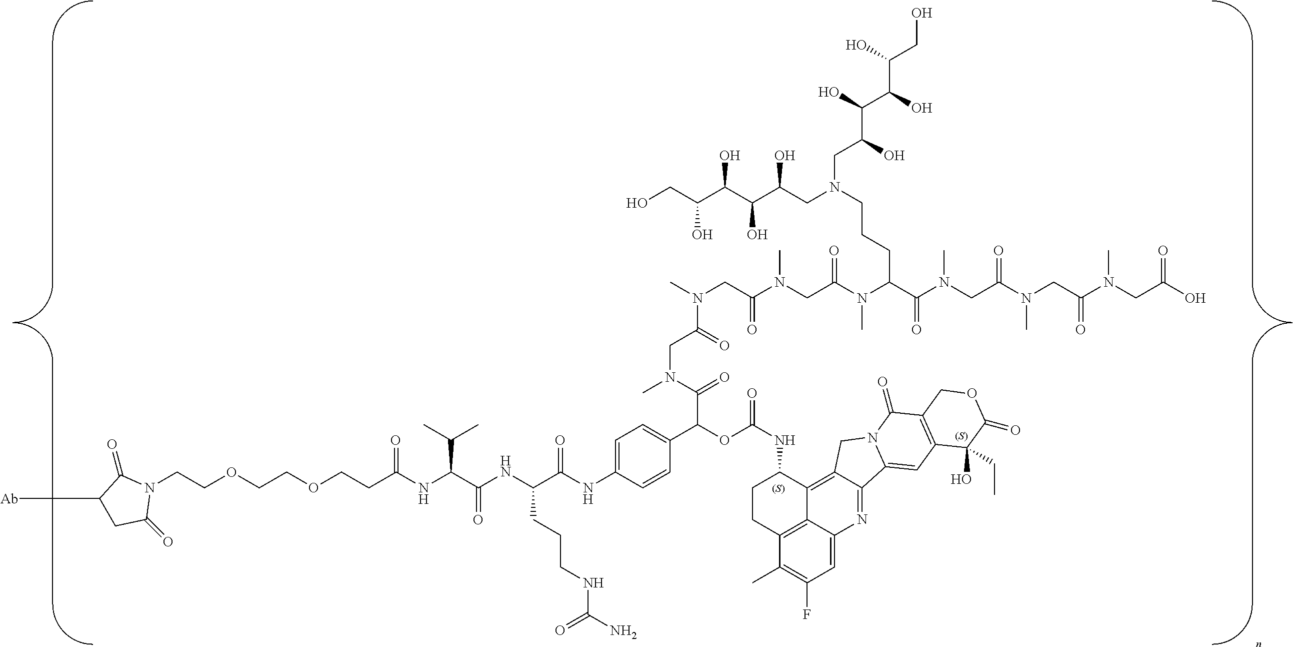 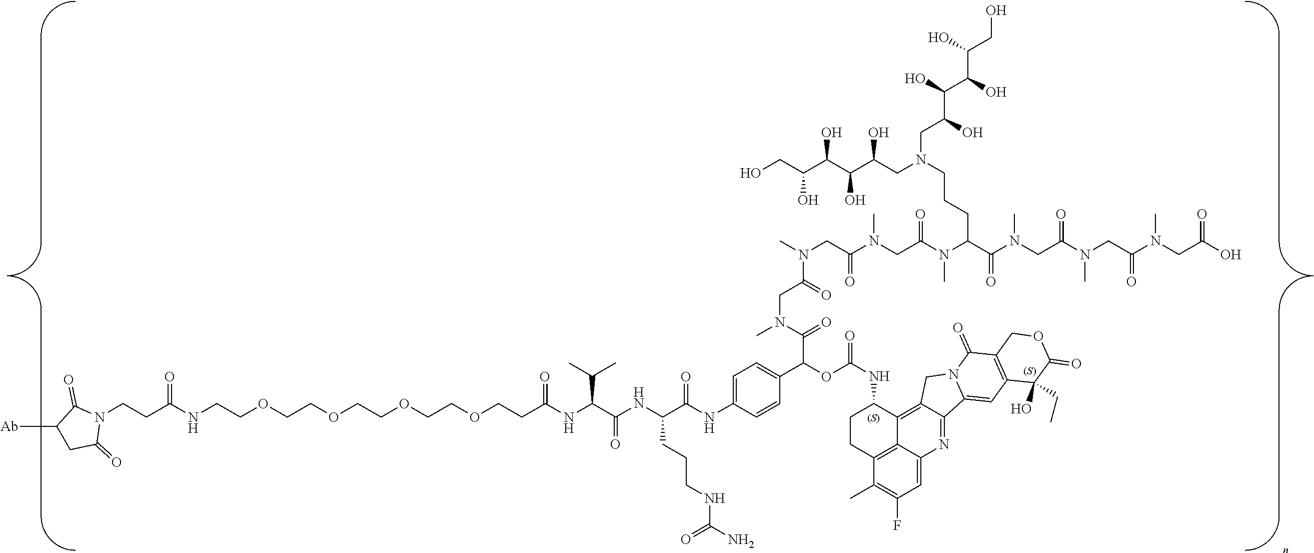 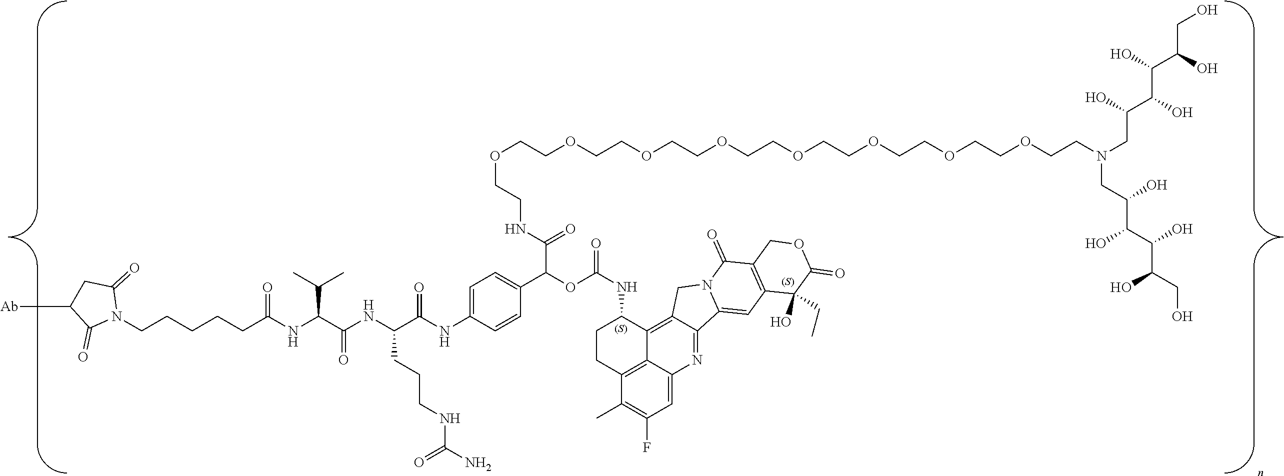 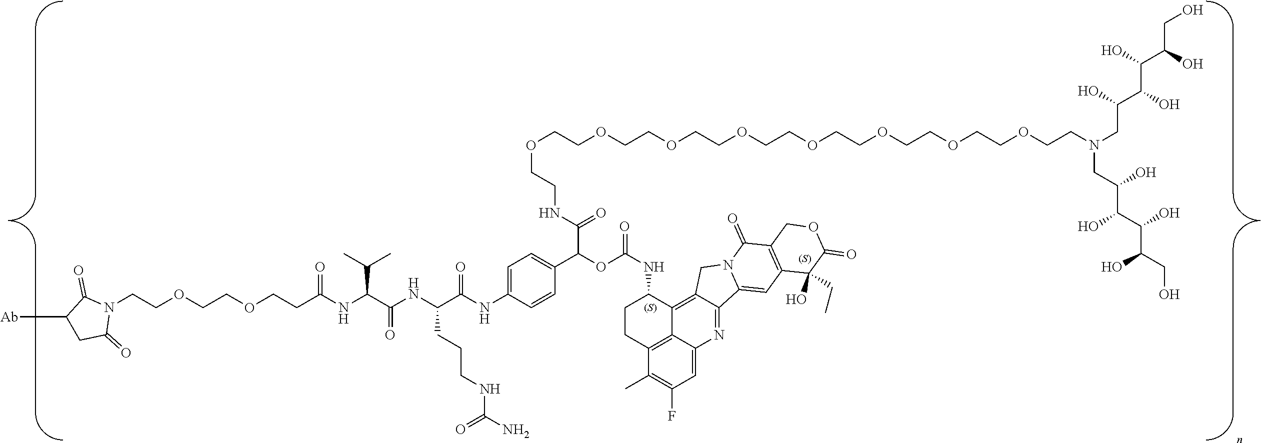 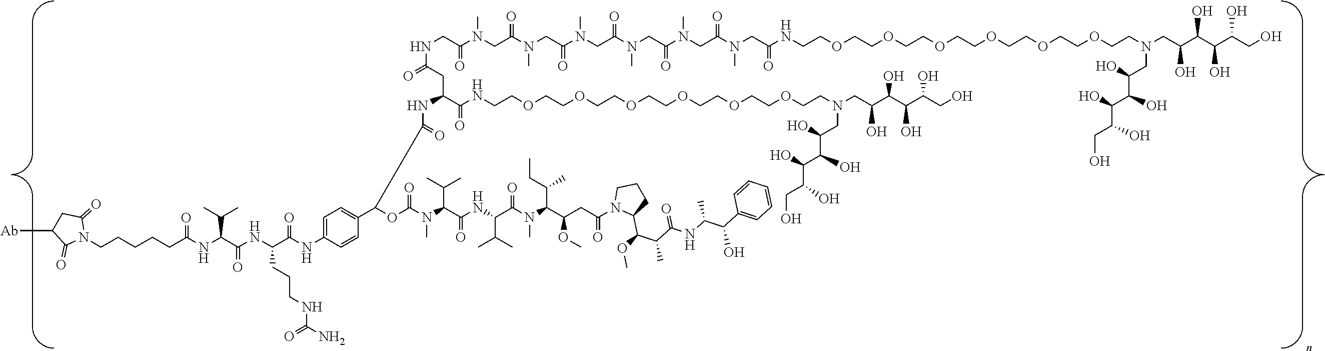 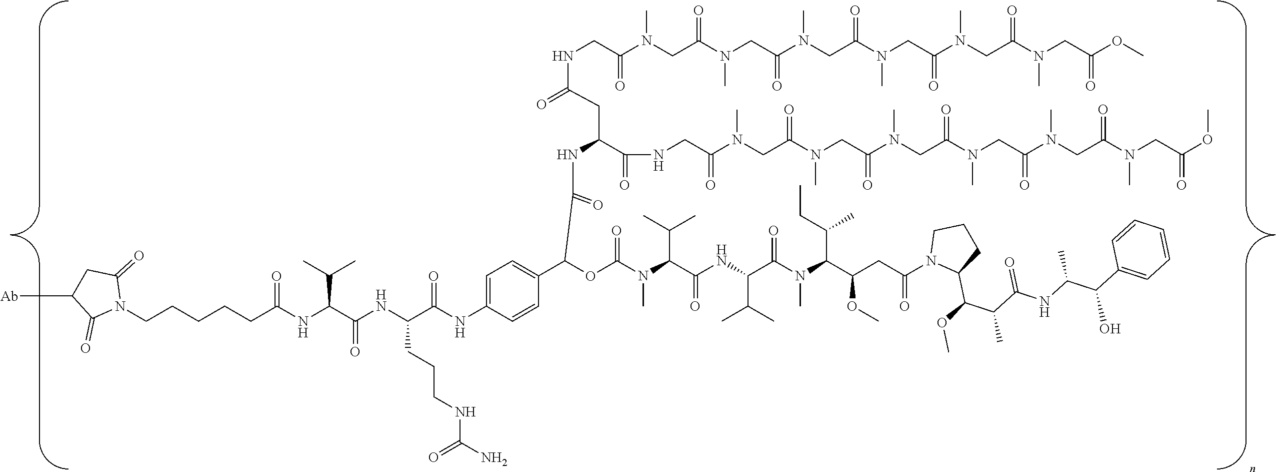 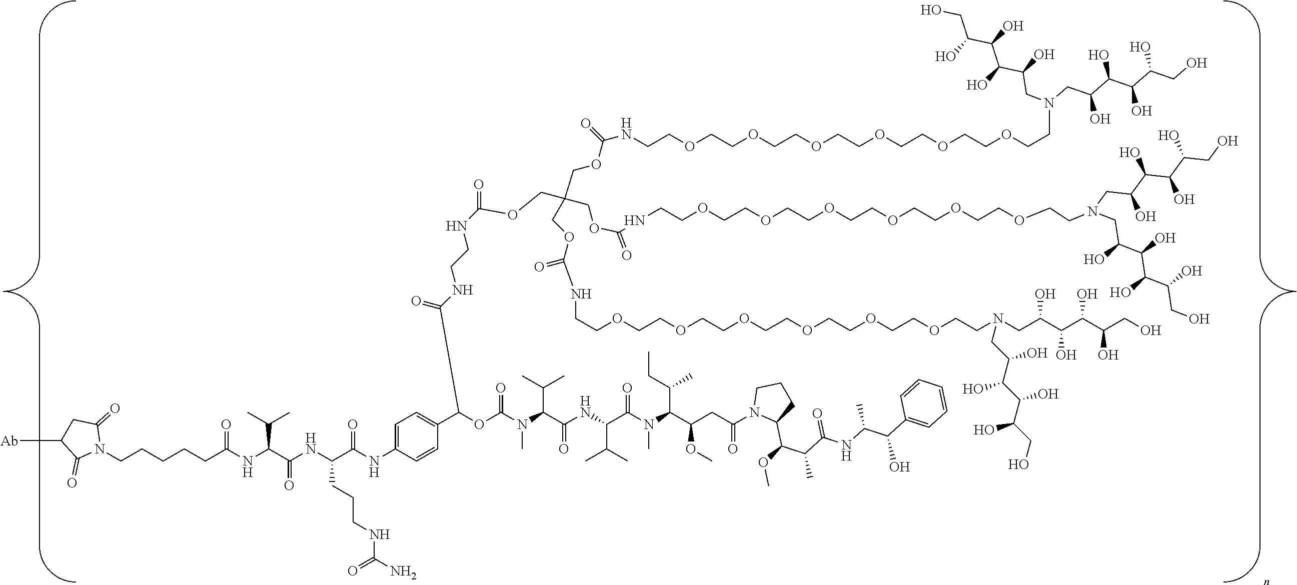 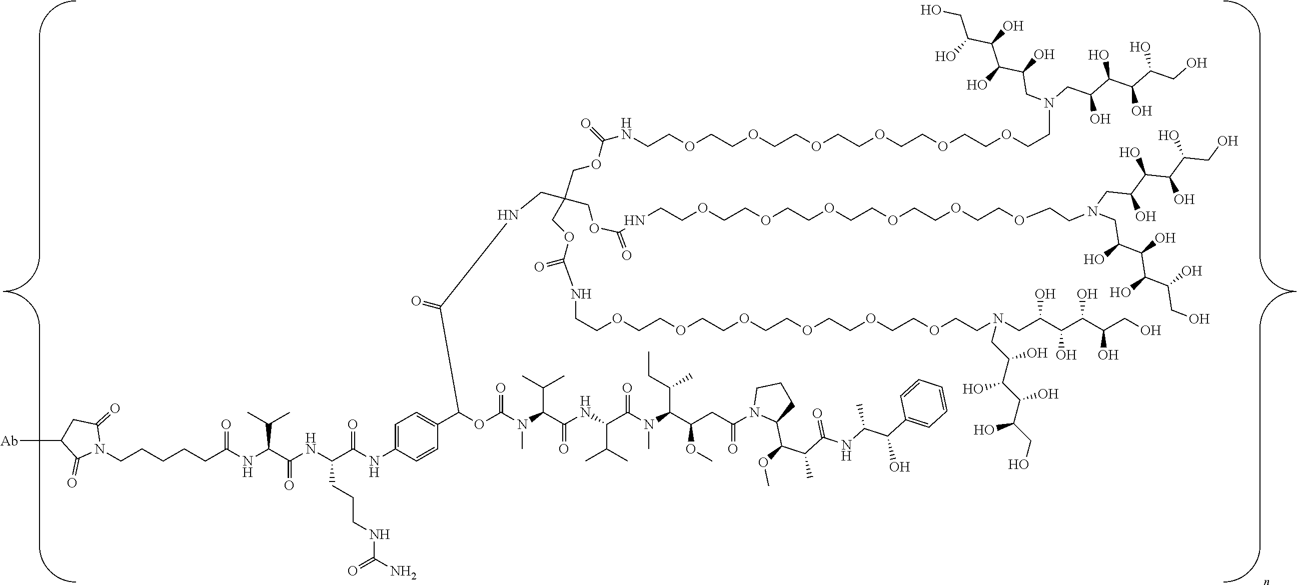 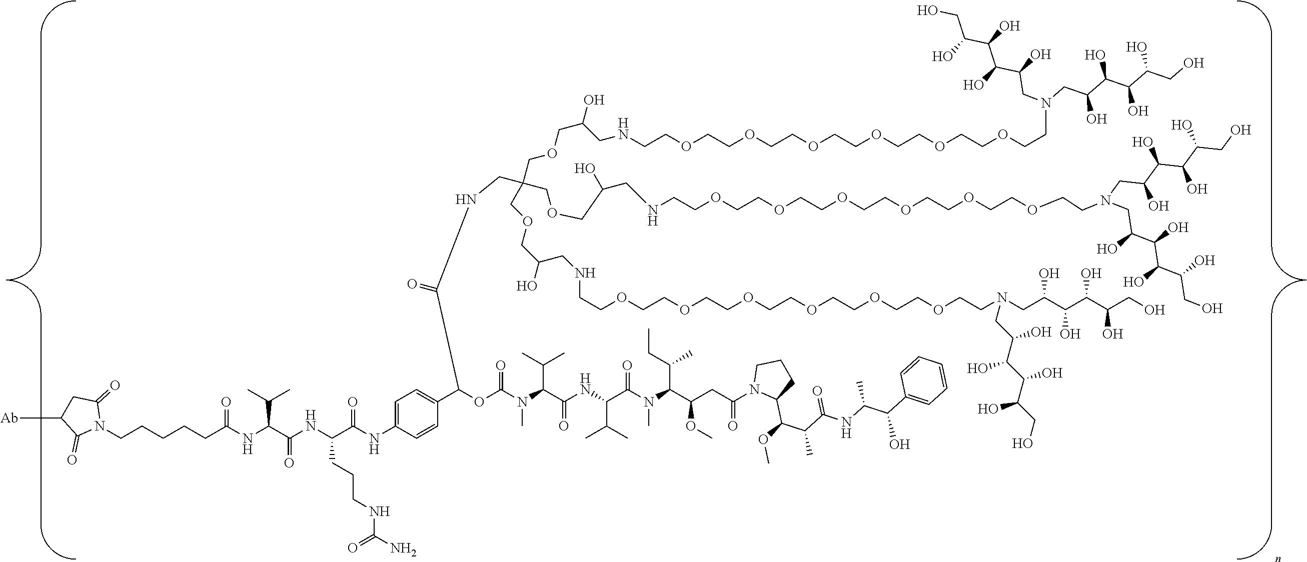 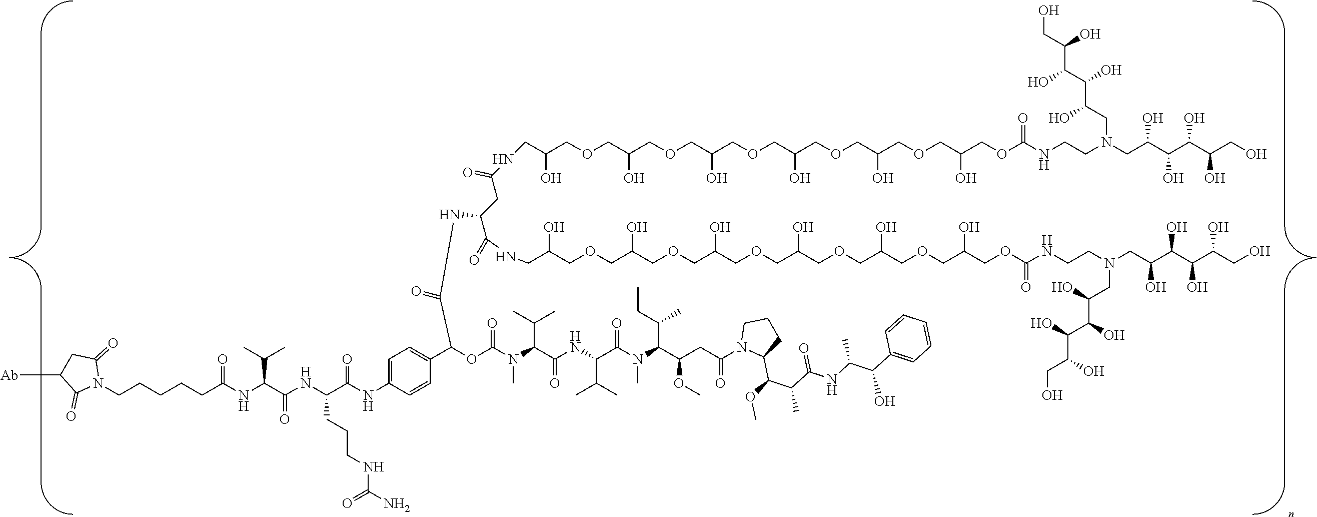 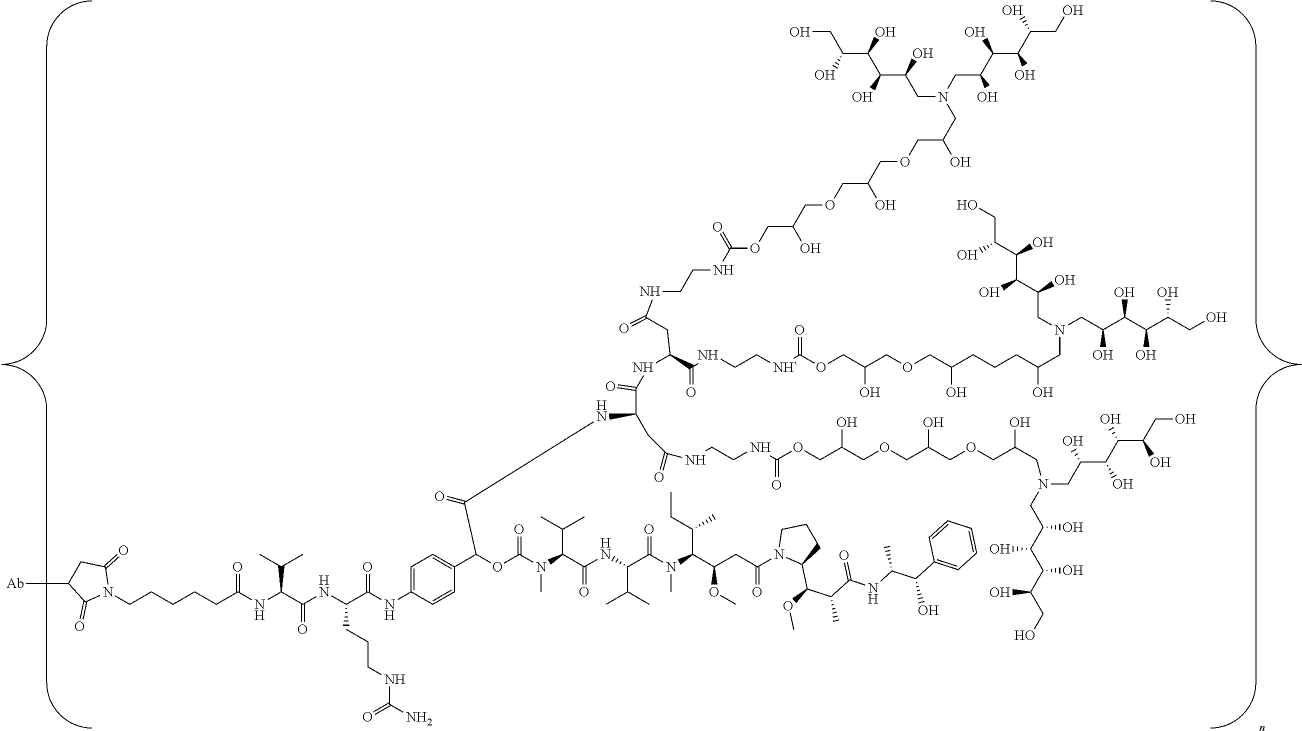 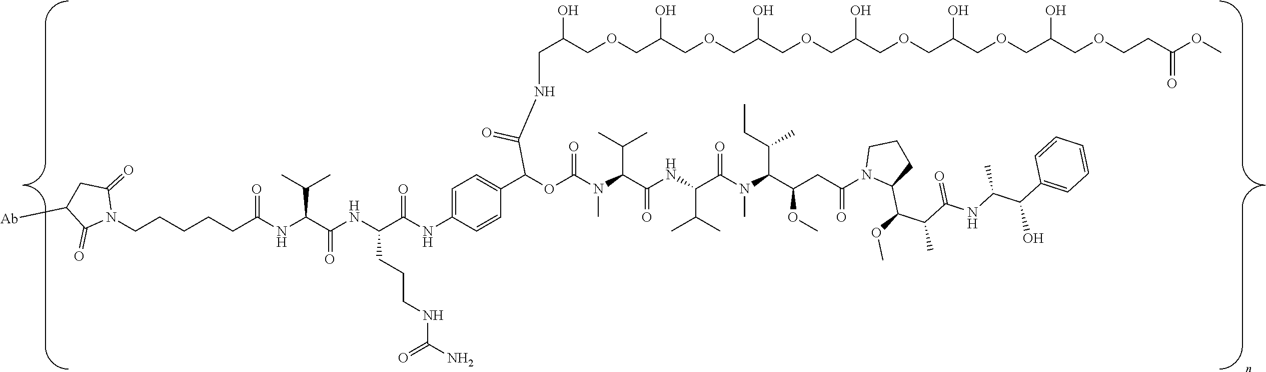 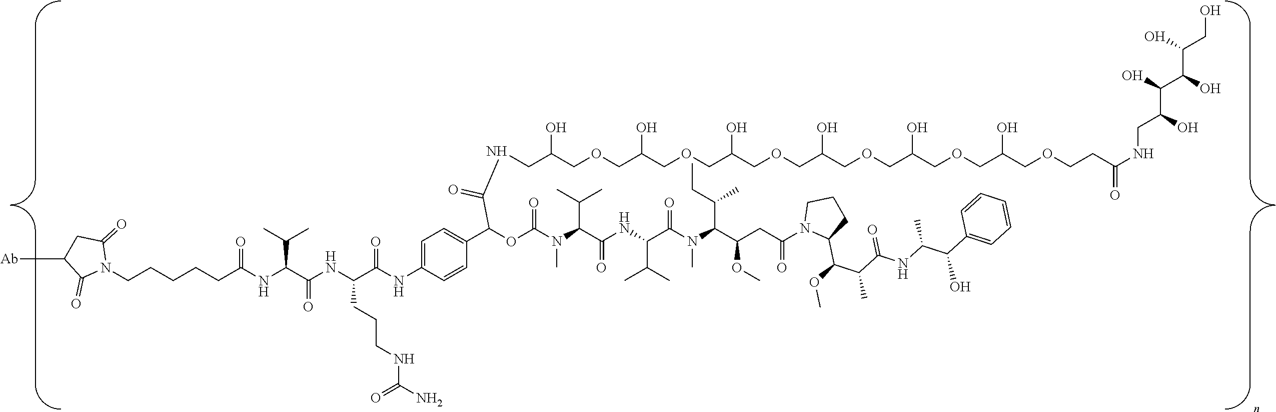 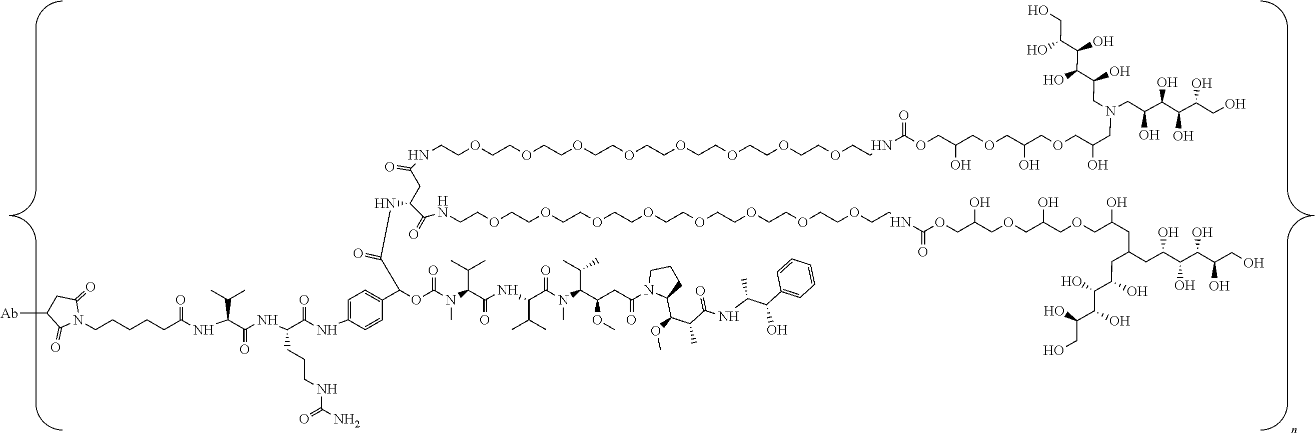 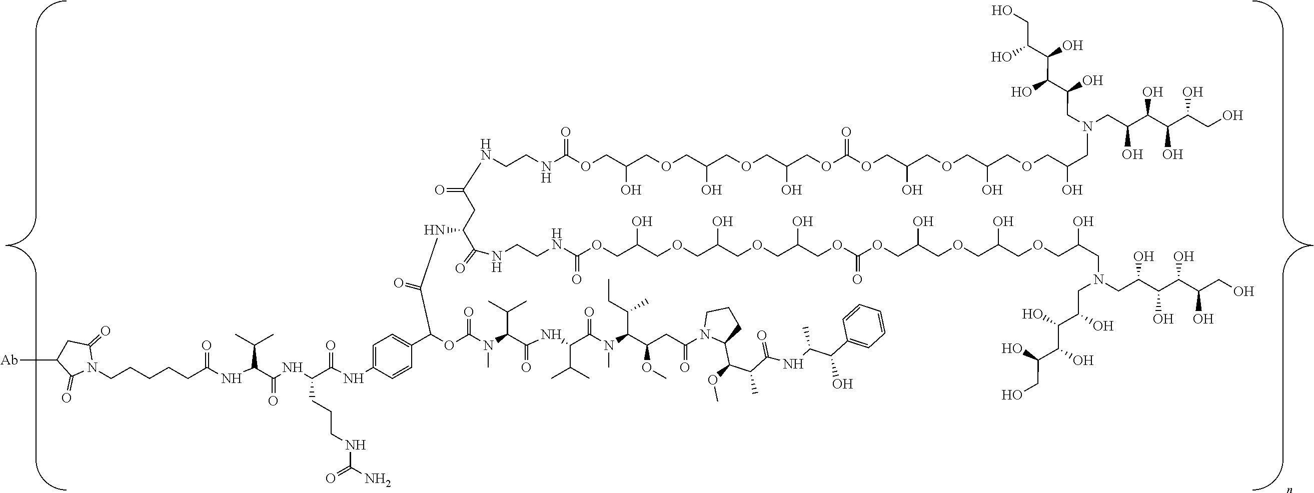 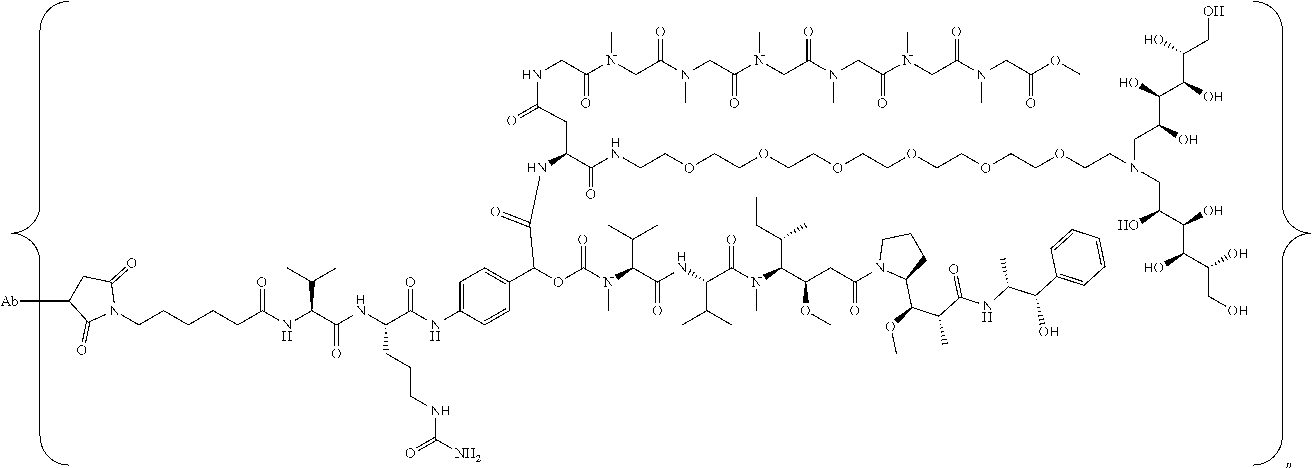 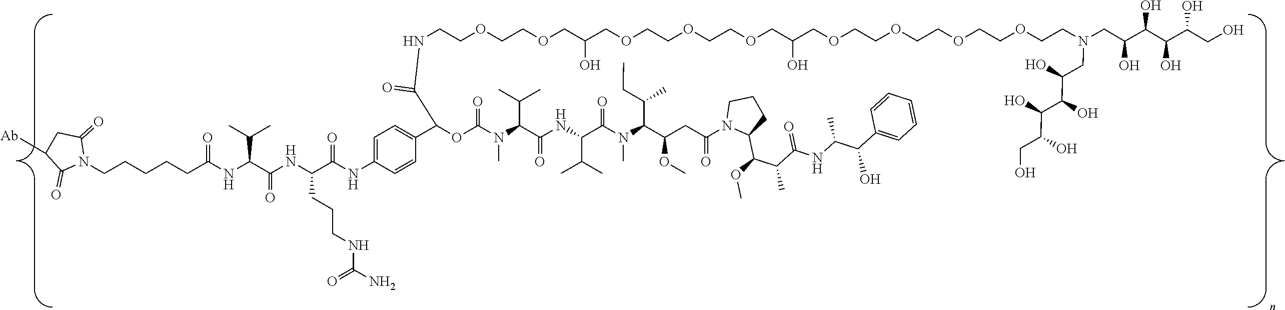 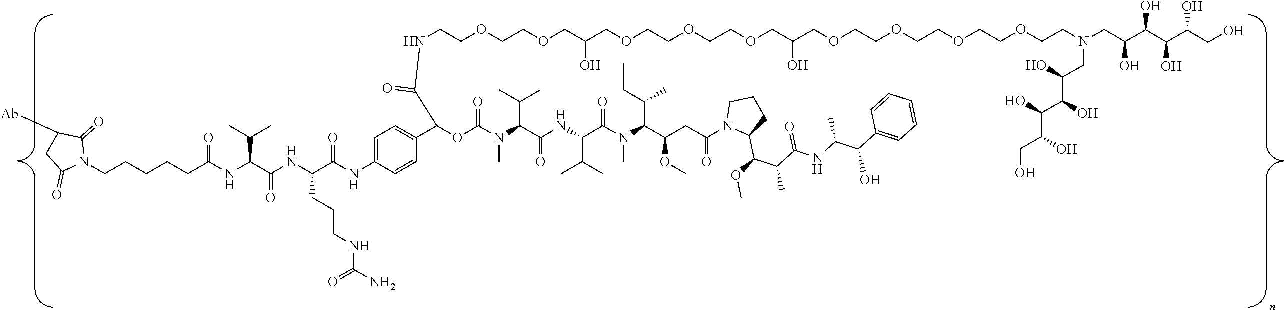 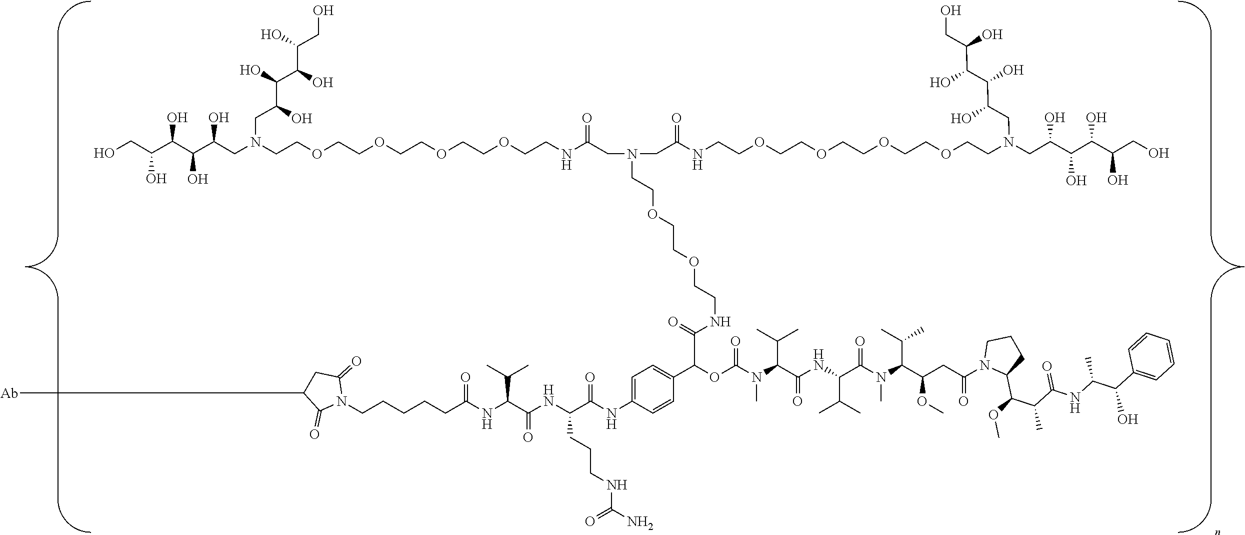 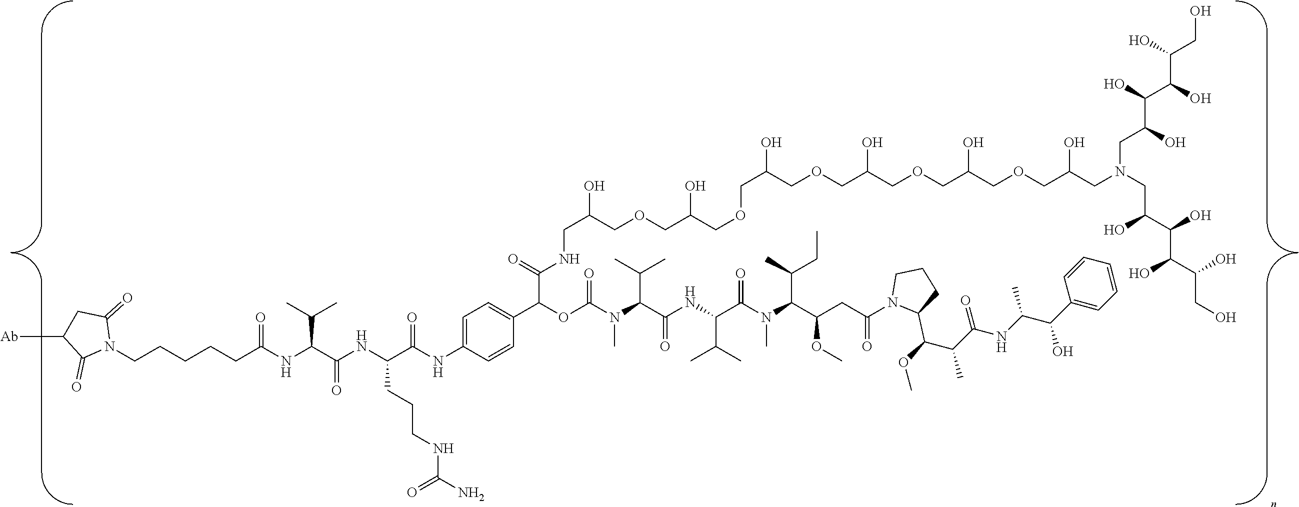 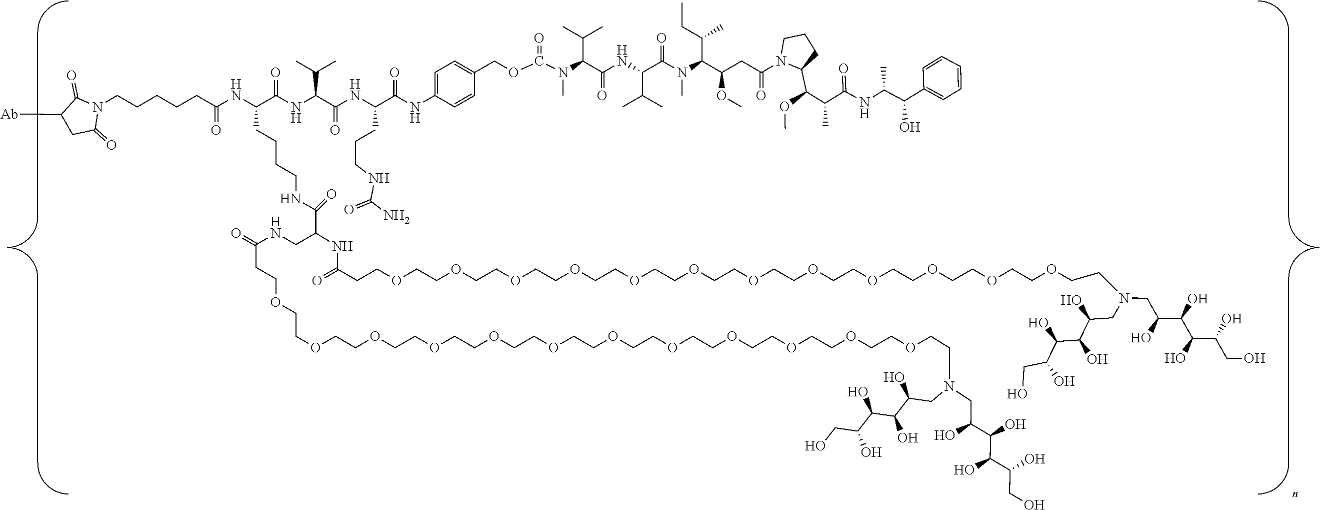 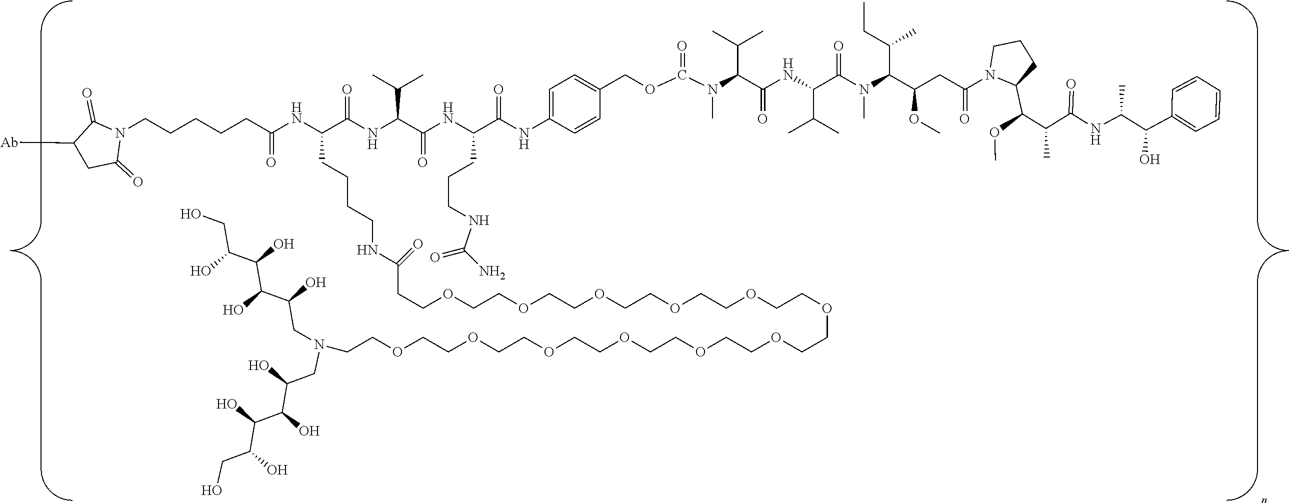 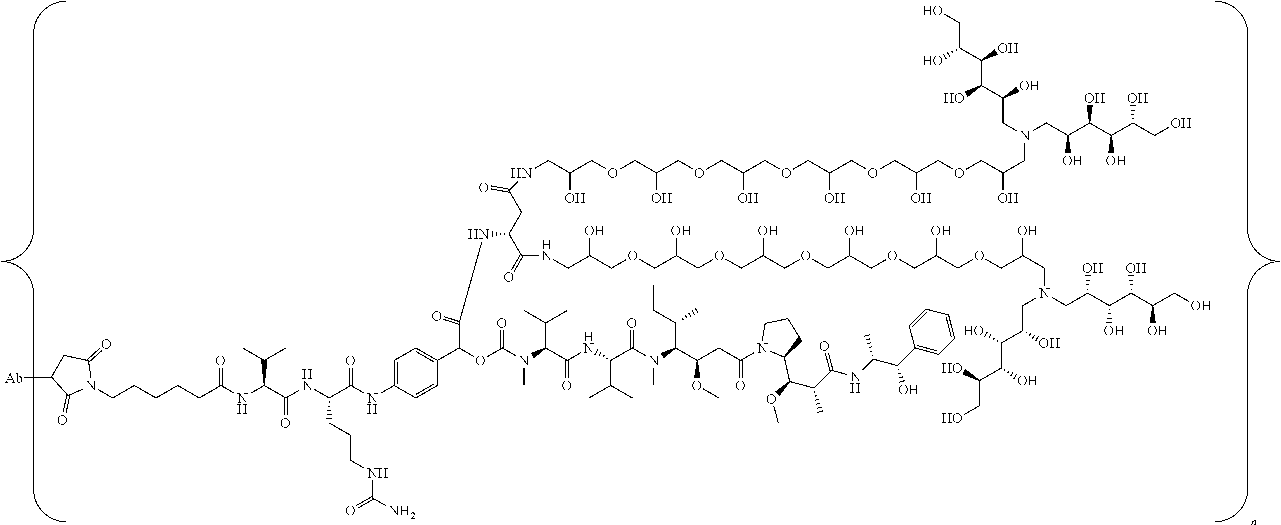 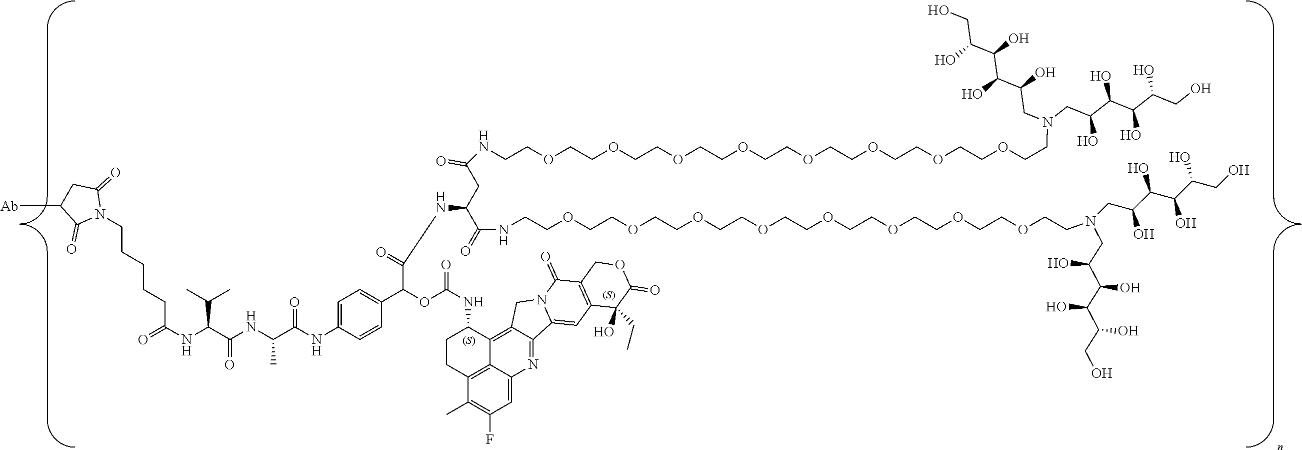 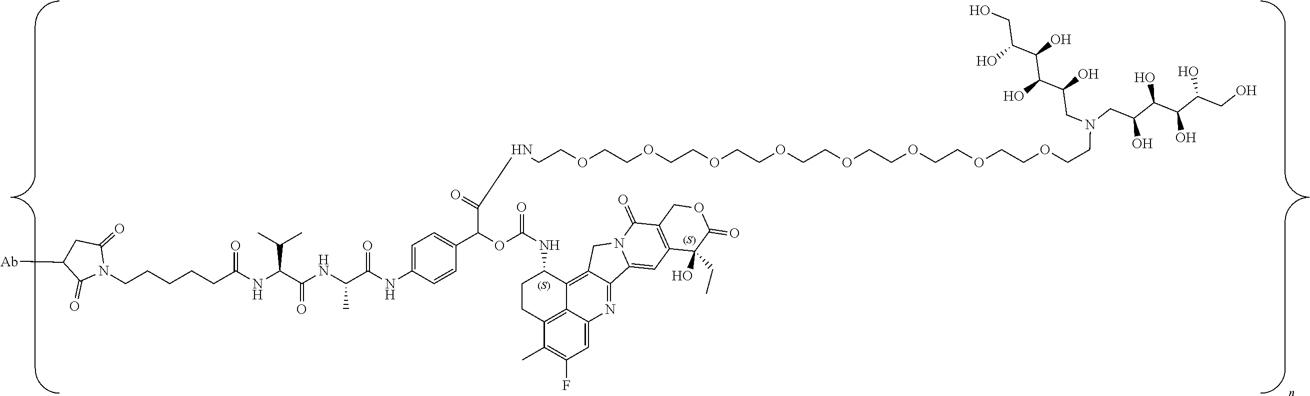 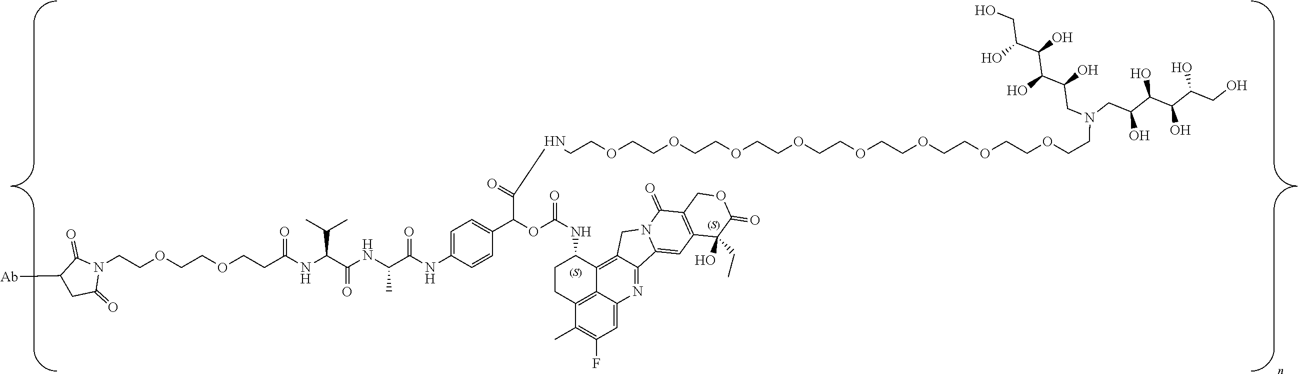 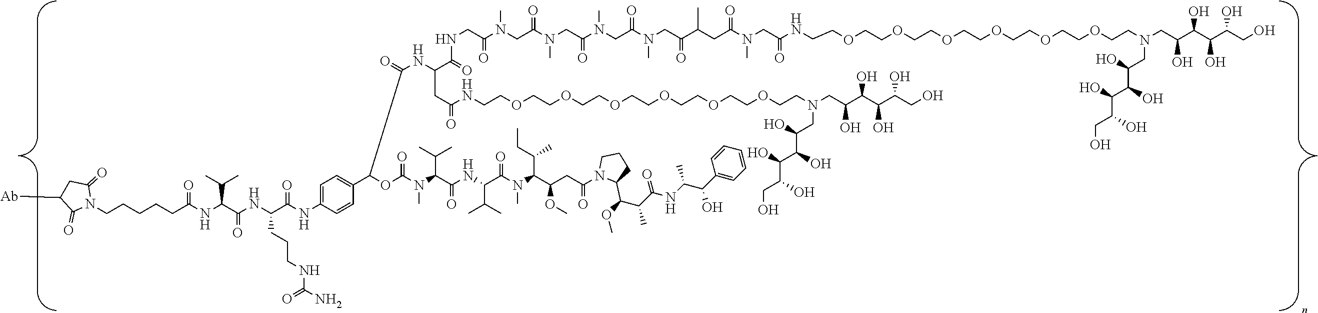 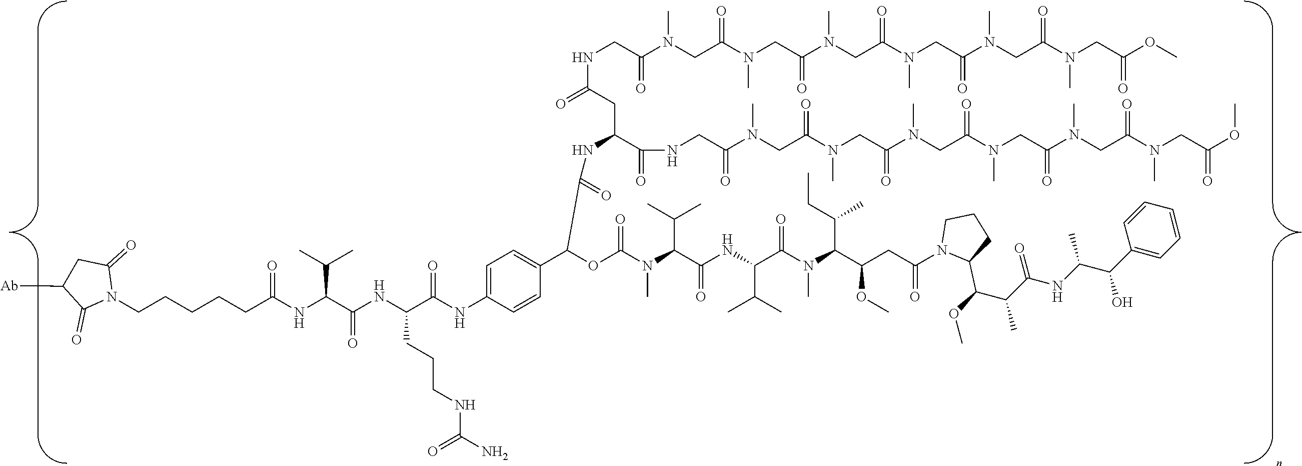 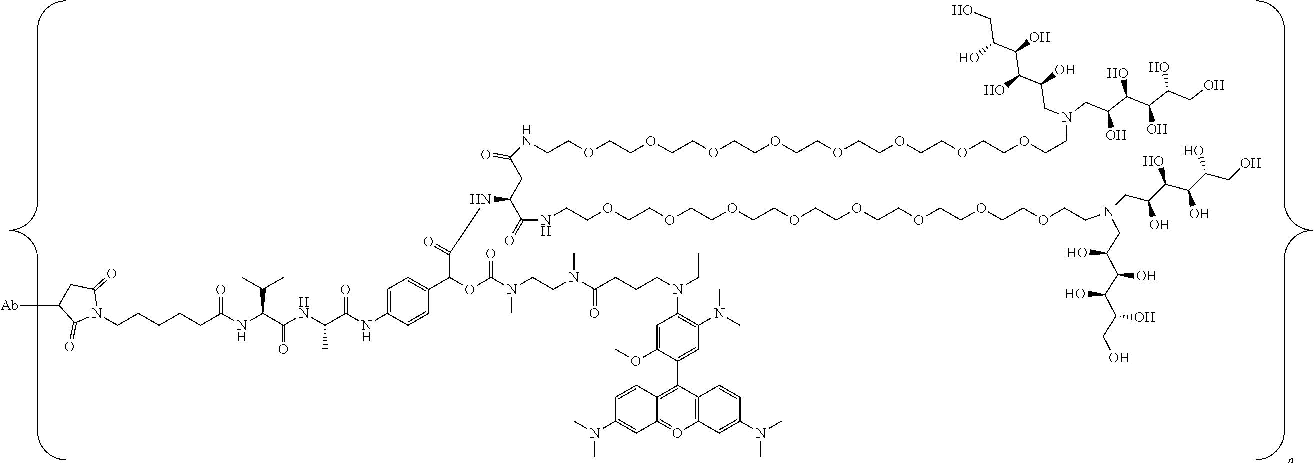 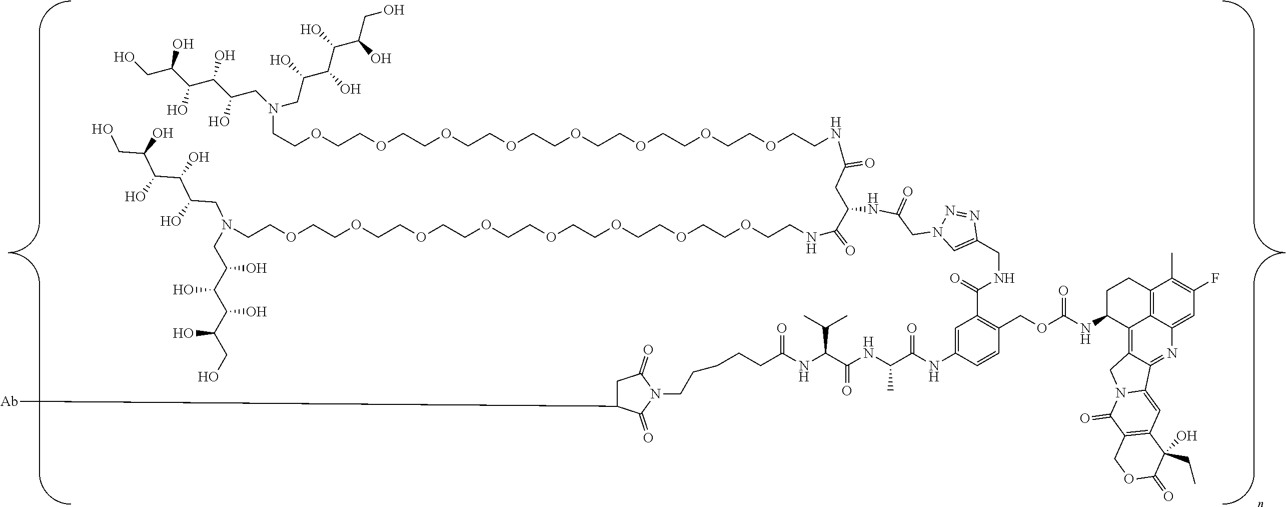 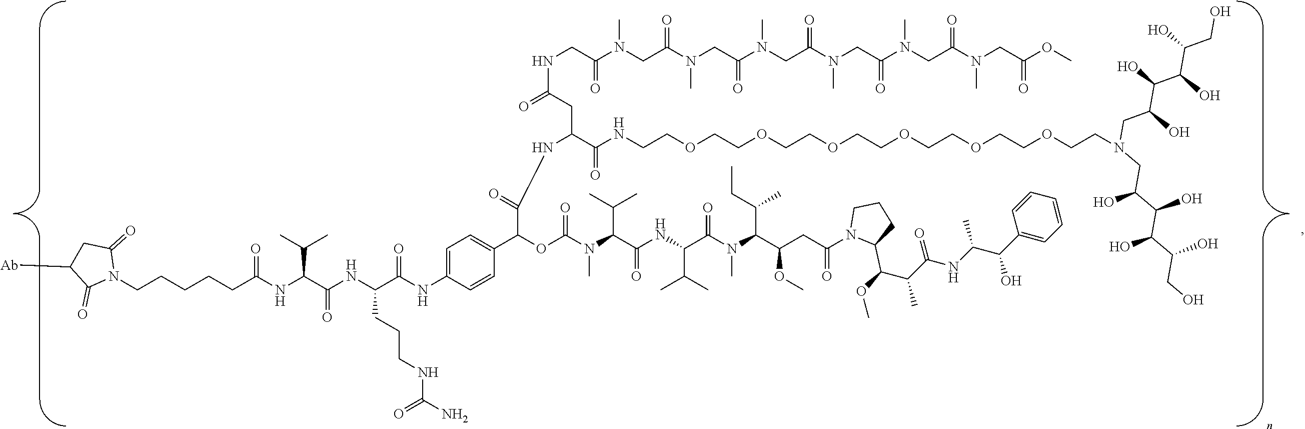
wherein Ab is the binding agent and n is p^{load}.

32. The conjugate of claim 26, wherein the conjugate has the following structure: 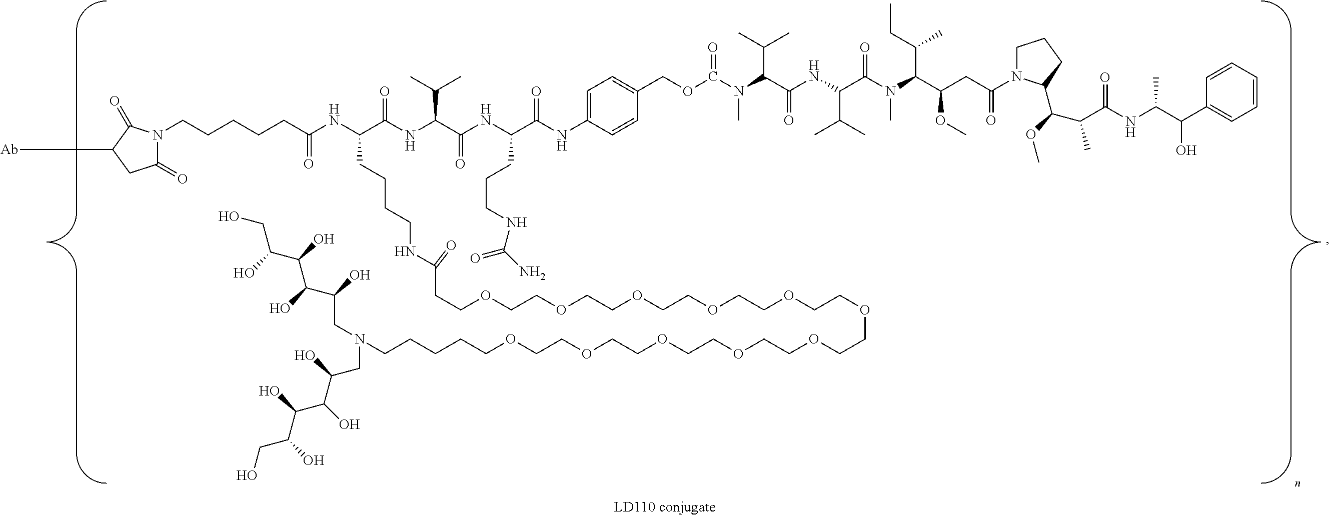 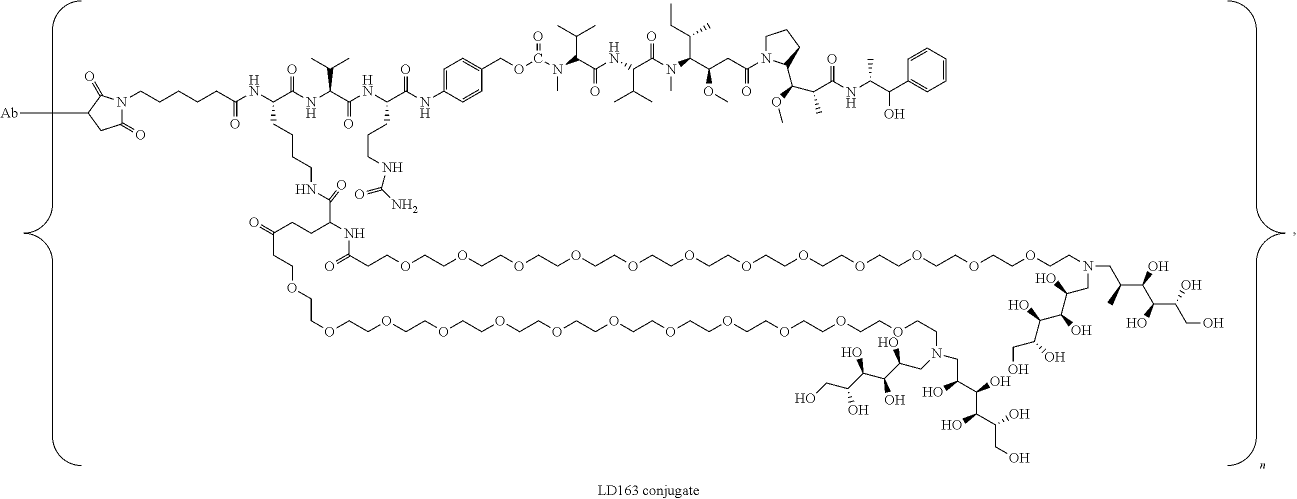 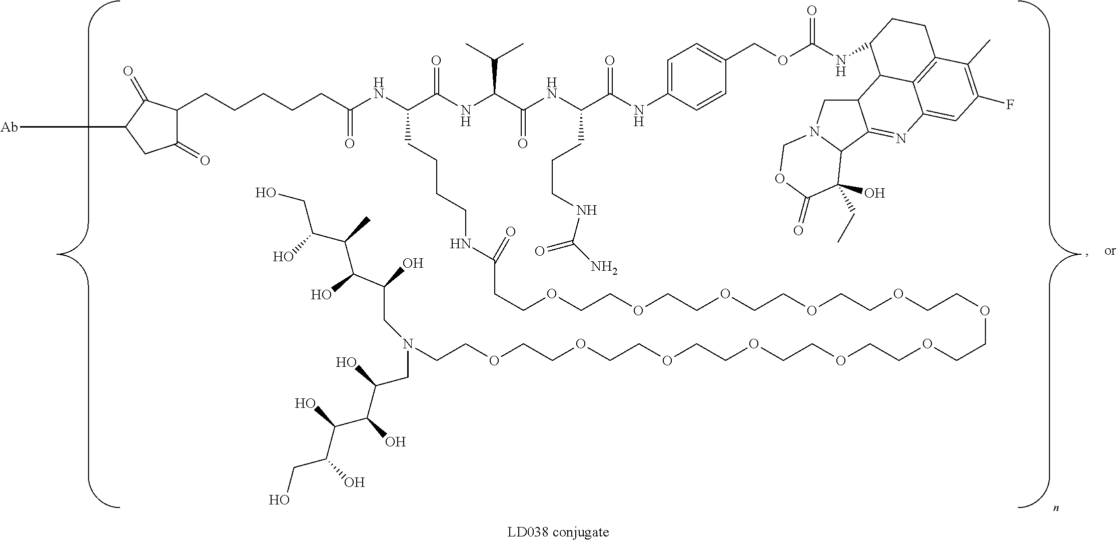 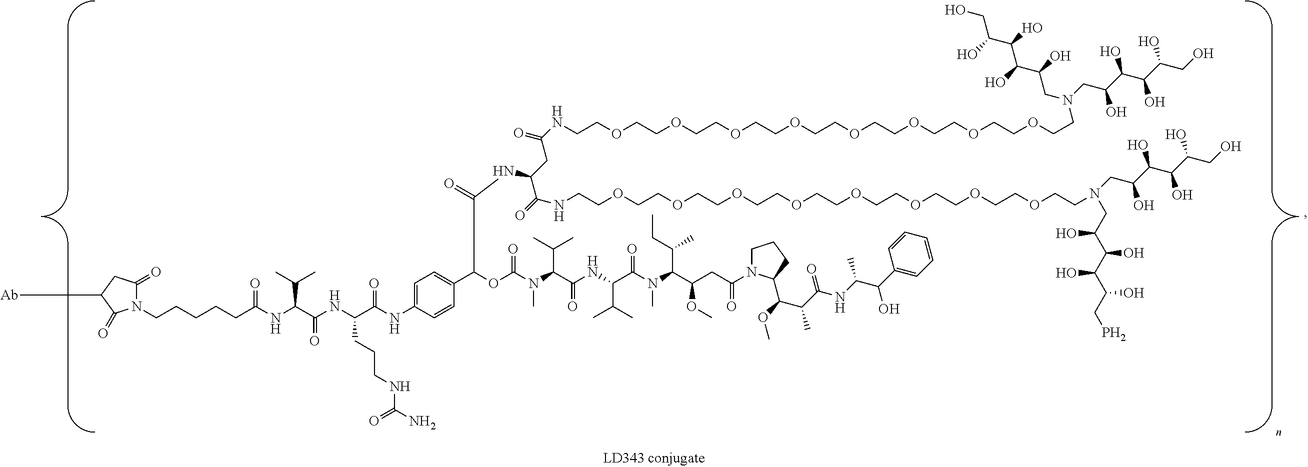
and wherein Ab is the binding agent and n is p^{load}, wherein p^{load} is from about 1 to about 8, about 2, about 4, about 6, about 8, about 10, about 12, about 14, about 16, about 3 to about 5, about 6 to about 8, or about 8 to about 16.

33. A conjugate comprising the following structure: 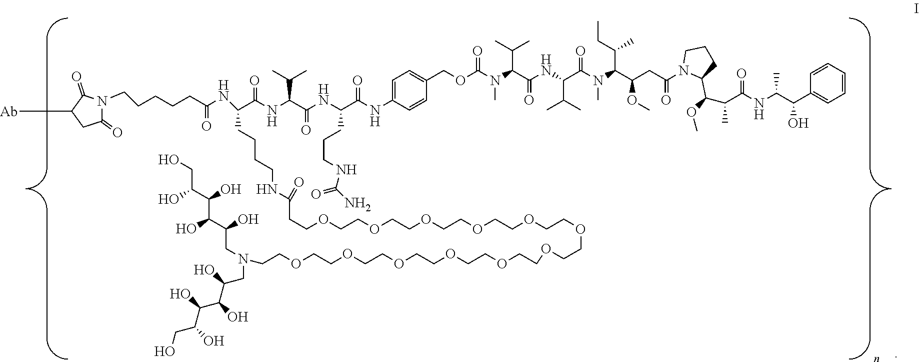
wherein Ab is an antibody, or antigen-binding portion thereof, which binds to SLIT and NTRK-like protein 6 (SLITRK6), wherein the antibody comprises a heavy chain variable (VH) region and a light chain variable (VL) region, wherein the VH region comprises the VHCDR1, VHCDR2, and VHCDR3 sequences set forth in SEQ ID NOs: 19, 20, and 21, respectively, and the VL region comprises the VLCDR1, VLCDR2, and VLCDR3 sequences set forth in SEQ ID NOs: 22, 23, and 24, respectively,
and wherein n is p^{load}, wherein p^{load} is from about 1 to about 8, about 2, about 4, about 6, about 8, about 10, about 12, about 14, about 16, about 3 to about 5, about 6 to about 8, or about 8 to about 16; 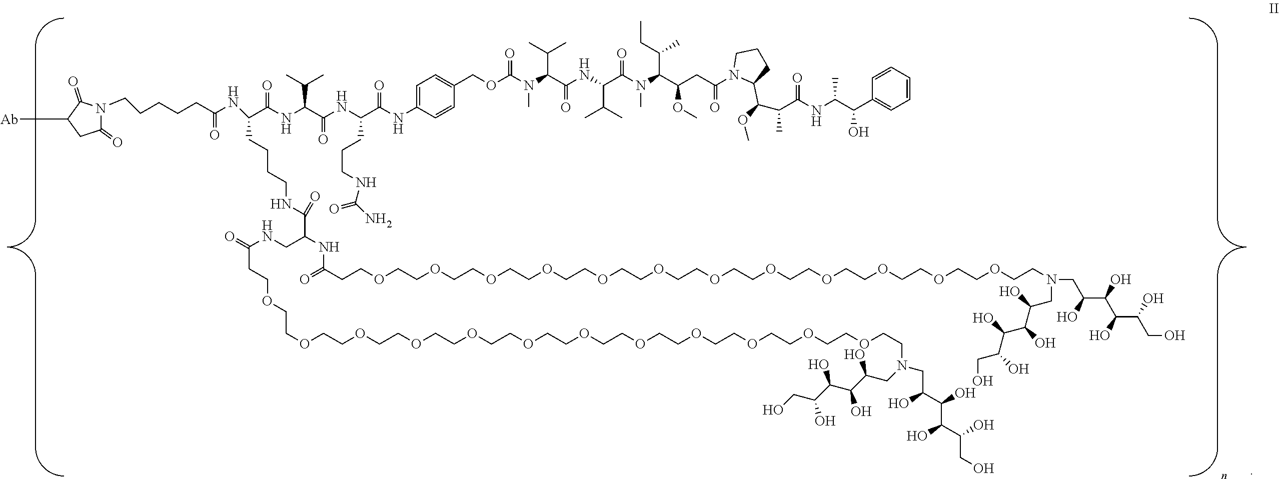
wherein Ab is an antibody, or antigen-binding portion thereof, which binds to SLIT and NTRK-like protein 6 (SLITRK6), wherein the antibody comprises a heavy chain variable (VH) region and a light chain variable (VL) region, wherein the VH region comprises the VHCDR1, VHCDR2, and VHCDR3 sequences set forth in SEQ ID NOs: 19, 20, and 21, respectively, and the VL region comprises the VLCDR1, VLCDR2, and VLCDR3 sequences set forth in SEQ ID NOs: 22, 23, and 24, respectively,
and wherein n is pload, wherein pload is from about 1 to about 8, about 2, about 4, about 6, about 8, about 10, about 12, about 14, about 16, about 3 to about 5, about 6 to about 8, or about 8 to about 16; 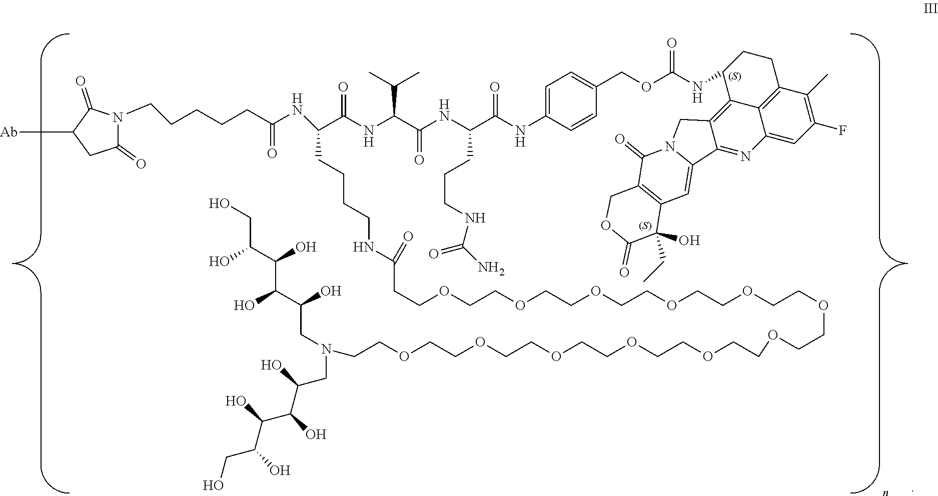
wherein Ab is an antibody, or antigen-binding portion thereof, which binds to SLIT and NTRK-like protein 6 (SLITRK6), wherein the antibody comprises a heavy chain variable (VH) region and a light chain variable (VL) region, wherein the VH region comprises the VHCDR1, VHCDR2, and VHCDR3 sequences set forth in SEQ ID NOs: 19, 20, and 21, respectively, and the VL region comprises the VLCDR1, VLCDR2, and VLCDR3 sequences set forth in SEQ ID NOs: 22, 23, and 24, respectively,
and wherein n is p^{load}, wherein p^{load} is from about 1 to about 8, about 2, about 4, about 6, about 8, about 10, about 12, about 14, about 16, about 3 to about 5, about 6 to about 8, or about 8 to about 16; or 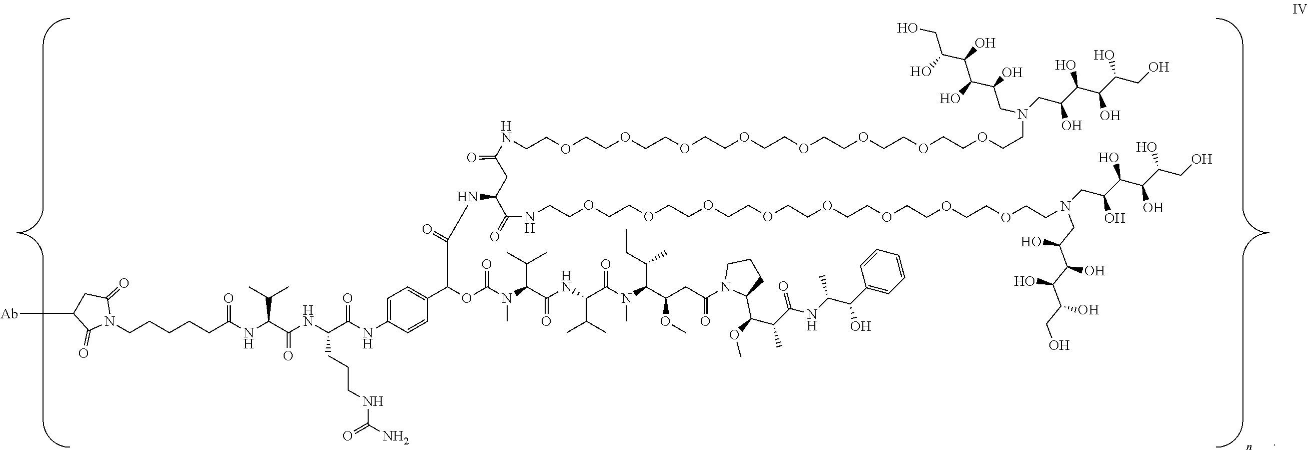
wherein Ab is an antibody, or antigen-binding portion thereof, which binds to SLIT and NTRK-like protein 6 (SLITRK6), wherein the antibody comprises a heavy chain variable (VH) region and a light chain variable (VL) region, wherein the VH region comprises the VHCDR1, VHCDR2, and VHCDR3 sequences set forth in SEQ ID NOs: 19, 20, and 21, respectively, and the VL region comprises the VLCDR1, VLCDR2, and VLCDR3 sequences set forth in SEQ ID NOs: 22, 23, and 24, respectively,
and wherein n is p^{load}, wherein p^{load} is from about 1 to about 8, about 2, about 4, about 6, about 8, about 10, about 12, about 14, about 16, about 3 to about 5, about 6 to about 8, or about 8 to about 16.

34-36. (canceled)

37. The conjugate of claim 33, wherein the VH and VL regions comprise the amino acid sequences set forth in SEQ ID NOs: 17 and 18, respectively.

38. The conjugate of claim 33, wherein the antibody comprises:
(a) a heavy chain and a light chain comprising the amino acid sequences of SEQ ID NOs: 53 and 55, respectively, or
(b) a heavy chain and a light chain comprising the amino acid sequences of SEQ ID NOs: 54 and 55, respectively.

39. (canceled)

40. A pharmaceutical composition comprising the binding agent of claim 1, or a conjugate comprising the binding agent, and a pharmaceutically acceptable carrier.

41. A method of treating cancer, preferably a SLITRK6+ cancer, comprising administering to a subject in need thereof a therapeutically effective amount of the binding agent of claim 1, a conjugate comprising the binding agent, or a pharmaceutical composition comprising the binding agent or conjugate.

42-45. (canceled)

46. The method of claim 41, wherein the SLITRK6+ cancer is a solid tumor or a hematologic malignancy.

47. The method of claim 41, wherein the SLITRK6+ cancer is selected from breast cancer (BC), lung cancer (LC), ovarian cancer (OVCA), esophageal cancer (EsC), gastric cancer (GC), bladder cancer (BLC), endometrial cancer (EC), head and neck cancer (HNC), cervical cancer, pharynx cancer, stomach cancer, myeloma, uterine cancer, colon cancer, hepatocellular cancer, and colorectal cancer.

48. A method of treating an autoimmune disease, comprising administering to a subject in need thereof a therapeutically effective amount of the binding agent of claim 1, a conjugate comprising the binding agent, or a pharmaceutical composition comprising the conjugate or binding agent.

49-50. (canceled)

51. The method of claim 48, wherein the autoimmune disease is rheumatoid arthritis, multiple sclerosis, or systemic lupus erythematosus.

52. A method of producing the binding agent which binds to SLITRK6, the method comprising culturing the cell line of claim 25 and isolating the binding agent from the cell.

53. An anti-idiotypic antibody which binds to the binding agent of claim 1.


SEQUENCE LISTING (58 sequences)

SEQ ID NO: 1 - protein, 117 aa, synthetic construct
  REGION 1..117: note = Synthetic: phA290 VH
QVQLVQSGAEVKKPGASVKVSCKASGYTFTSYYMHWVRQAPGQGLEWMGIINPSGGSTSYAQKFQGRVTMTRDTSTSTVYMELSSLRSEDTAVYYCARGFTRYFDYWGQGTLVTVSA

SEQ ID NO: 2 - protein, 107 aa, synthetic construct
  REGION 1..107: note = Synthetic: phA290 VL
EIVMTQSPGTLSLSPGERATLSCRASQSVSKYLAWYQQRPGQAPRLLIYDASNRATGIPARFSGSGSGTDFTLTVSSLEPEDFAVYYCQQRYNWPLTFGGGTTVEIK

SEQ ID NO: 3 - protein, 5 aa, synthetic construct
  REGION 1..5: note = Synthetic: phA290 HCDR1
SYYMH

SEQ ID NO: 4 - protein, 17 aa, synthetic construct
  REGION 1..17: note = Synthetic: phA290 HCDR2
IINPSGGSTSYAQKFQG

SEQ ID NO: 5 - protein, 8 aa, synthetic construct
  REGION 1..8: note = Synthetic: phA290 HCDR3
GFTRYFDY

SEQ ID NO: 6 - protein, 11 aa, synthetic construct
  REGION 1..11: note = Synthetic: phA290 LCDR1
RASQSVSKYLA

SEQ ID NO: 7 - protein, 7 aa, synthetic construct
  REGION 1..7: note = Synthetic: phA290 LCDR2
DASNRAT

SEQ ID NO: 8 - protein, 9 aa, synthetic construct
  REGION 1..9: note = Synthetic: phA290 LCDR3
QQRYNWPLT

SEQ ID NO: 9 - protein, 120 aa, synthetic construct
  REGION 1..120: note = Synthetic: phA108 VH
QVQLVQSGAEVKKPGASVKVSCTASGYIFNDYYLHWVRQAPGQGLEWVGIINPSGGSPVYAQKFQGRVTMTRDTSTNTVYMELSSLRSEDTAVYYCARGMVRGVRLFDYWGQGTLVTVSA

SEQ ID NO: 10 - protein, 107 aa, synthetic construct
  REGION 1..107: note = Synthetic: phA108 VL
EIVLTQSPATLSLSPGERATLSCRASQSVSSYLAWYQQKPGQTPRLLIYDASNRATGVPARFSGSGSGTDFTLTISSLETEDFAVYYCQQRTNWPLTFGGGTKVEIK

SEQ ID NO: 11 - protein, 5 aa, synthetic construct
  REGION 1..5: note = Synthetic: phA108 HCDR1
DYYLH

SEQ ID NO: 12 - protein, 17 aa, synthetic construct
  REGION 1..17: note = Synthetic: phA108 HCDR2
IINPSGGSPVYAQKFQG

SEQ ID NO: 13 - protein, 11 aa, synthetic construct
  REGION 1..11: note = Synthetic: phA108 HCDR3
GMVRGVRLFDY

SEQ ID NO: 14 - protein, 11 aa, synthetic construct
  REGION 1..11: note = Synthetic: phA108 LCDR1
RASQSVSSYLA

SEQ ID NO: 15 - protein, 7 aa, synthetic construct
  REGION 1..7: note = Synthetic: phA108 LCDR2
DASNRAT

SEQ ID NO: 16 - protein, 9 aa, synthetic construct
  REGION 1..9: note = Synthetic: phA108 LCDR3
QQRTNWPLT

SEQ ID NO: 17 - protein, 116 aa, synthetic construct
  REGION 1..116: note = Synthetic: hu1H2-03 VH
EVQLVESGGGLVQPGGSLRLSCAASGFTFSDYGLHWVRQAPGKGLEWVSYISSGSSTVYFADTLKGRFTISRDNAKNSLYLQMNSLRAEDTAVYYCTRGTWYFDVWGRGTLVTVSS

SEQ ID NO: 18 - protein, 107 aa, synthetic construct
  REGION 1..107: note = Synthetic: hu1H2-03 VL
DIQMTQSPSSLSASVGDRVTITCRTSENIHSYLAWYQQKPGKSPQLLVYNAKTLADGVPSRFSGSGSGTDYTLTISSLQPEDFGTFYCQHFWTTSRTFGGGTKVEIK

SEQ ID NO: 19 - protein, 5 aa, synthetic construct
  REGION 1..5: note = Synthetic: hu1H2-03 HCDR1
DYGLH

SEQ ID NO: 20 - protein, 17 aa, synthetic construct
  REGION 1..17: note = Synthetic: hu1H2-03 HCDR2
YISSGSSTVYFADTLKG

SEQ ID NO: 21 - protein, 7 aa, synthetic construct
  REGION 1..7: note = Synthetic: hu1H2-03 HCDR3
GTWYFDV

SEQ ID NO: 22 - protein, 11 aa, synthetic construct
  REGION 1..11: note = Synthetic: hu1H2-03 LCDR1
RTSENIHSYLA

SEQ ID NO: 23 - protein, 7 aa, synthetic construct
  REGION 1..7: note = Synthetic: hu1H2-03 LCDR2
NAKTLAD

SEQ ID NO: 24 - protein, 9 aa, synthetic construct
  REGION 1..9: note = Synthetic: hu1H2-03 LCDR3
QHFWTTSRT

SEQ ID NO: 25 - protein, 116 aa, synthetic construct
  REGION 1..116: note = Synthetic: hu1H2-13 VH
EVQLVESGGGLVQPGGSLRLSCAASGFTFSDYGLHWVRQAPGKGLEWVAYISSGSSTVYFADTLKGRFTISRDNAKNSLYLQMNSLRAEDTAVYYCTRGTWYFDVWGRGTLVTVSS

SEQ ID NO: 26 - protein, 107 aa, synthetic construct; identical to SEQ ID NO: 18
  REGION 1..107: note = Synthetic: hu1H2-13 VL

SEQ ID NO: 27 - protein, 5 aa, synthetic construct
  REGION 1..5: note = Synthetic: hu1H2-13 HCDR1
DYGLH

SEQ ID NO: 28 - protein, 17 aa, synthetic construct
  REGION 1..17: note = Synthetic: hu1H2-13 HCDR2
YISSGSSTVYFADTLKG

SEQ ID NO: 29 - protein, 7 aa, synthetic construct
  REGION 1..7: note = Synthetic: hu1H2-13 HCDR3
GTWYFDV

SEQ ID NO: 30 - protein, 11 aa, synthetic construct
  REGION 1..11: note = Synthetic: hu1H2-13 LCDR1
RTSENIHSYLA

SEQ ID NO: 31 - protein, 7 aa, synthetic construct
  REGION 1..7: note = Synthetic: hu1H2-13 LCDR2
NAKTLAD

SEQ ID NO: 32 - protein, 9 aa, synthetic construct
  REGION 1..9: note = Synthetic: hu1H2-13 LCDR3
QHFWTTSRT

SEQ ID NO: 33 - protein, 120 aa, synthetic construct
  REGION 1..120: note = Synthetic: hu14G1-03 VH
EVQLVESGGGLVQPGGSLRLSCAASGFTFSDYGMHWVRQAPGKGLEWVSYISPGSSTIYYADTMKGRFTISRDNAKNSLYLQMNSLRAEDTAVYYCARAYGGSYWYFAVWGQGTLVTVSS

SEQ ID NO: 34 - protein, 107 aa, synthetic construct
  REGION 1..107: note = Synthetic: hu14G1-03 VL
DIQMTQSPSSLSASVGDRVTITCRASGNIHSYLAWYQQKPGKSPQLLVYNAKTLADGVPSRFSGSGSGTDYTLTISSLQPEDFGTYYCQHFWSTPYTFGGGTKLEIK

SEQ ID NO: 35 - protein, 5 aa, synthetic construct
  REGION 1..5: note = Synthetic: hu14G1-03 HCDR1
DYGMH

SEQ ID NO: 36 - protein, 17 aa, synthetic construct
  REGION 1..17: note = Synthetic: hu14G1-03 HCDR2
YISPGSSTIYYADTMKG

SEQ ID NO: 37 - protein, 11 aa, synthetic construct
  REGION 1..11: note = Synthetic: hu14G1-03 HCDR3
AYGGSYWYFAV

SEQ ID NO: 38 - protein, 11 aa, synthetic construct
  REGION 1..11: note = Synthetic: hu14G1-03 LCDR1
RASGNIHSYLA

SEQ ID NO: 39 - protein, 7 aa, synthetic construct
  REGION 1..7: note = Synthetic: hu14G1-03 LCDR2
NAKTLAD

SEQ ID NO: 40 - protein, 9 aa, synthetic construct
  REGION 1..9: note = Synthetic: hu14G1-03 LCDR3
QHFWSTPYT

SEQ ID NO: 41 - protein, 119 aa, synthetic construct
  REGION 1..119: note = Synthetic: Sirtratuzumab VH
QVQLVESGGGVVQPGRSLRLSCAASGFTFSSYGMHWVRQAPGKGLEWVAVIWYDGSNQYYADSVKGRFTISRDNSKNTLFLQMHSLRAEDTAVYYCARGLTSGRYGMDWGQGTTVTVSS

SEQ ID NO: 42 - protein, 112 aa, synthetic construct
  REGION 1..112: note = Synthetic: Sirtratuzumab VL
DIVMTQSPLSLPVTPGEPASISCRSSQSLLLSHGFNYLDWYLQKPGQSPQLLIYLGSSRASGVPDRFSGSGSGTDFTLKISRVEAEDVGLYYCMQPLQIPWTFGQGTKVEIK

SEQ ID NO: 43 - protein, 5 aa, synthetic construct
  REGION 1..5: note = Synthetic: Sirtratuzumab HCDR1
SYGMH

SEQ ID NO: 44 - protein, 17 aa, synthetic construct
  REGION 1..17: note = Synthetic: Sirtratuzumab HCDR2
VIWYDGSNQYYADSVKG

SEQ ID NO: 45 - protein, 10 aa, synthetic construct
  REGION 1..10: note = Synthetic: Sirtratuzumab HCDR3
GLTSGRYGMD

SEQ ID NO: 46 - protein, 14 aa, synthetic construct
  REGION 1..14: note = Synthetic: Sirtratuzumab LCDR1
RSSQSLLLSHNYLD

SEQ ID NO: 47 - protein, 7 aa, synthetic construct
  REGION 1..7: note = Synthetic: Sirtratuzumab LCDR2
LGSSRAS

SEQ ID NO: 48 - protein, 9 aa, synthetic construct
  REGION 1..9: note = Synthetic: Sirtratuzumab LCDR3
MQPLQIPWT

SEQ ID NO: 49 - protein, 330 aa, Homo sapiens [sequence omitted]

SEQ ID NO: 50 - protein, 107 aa, Homo sapiens [sequence omitted]

SEQ ID NO: 51 - protein, 330 aa, synthetic construct
  REGION 1..330: note = Synthetic: Heavy chain constant chain with Fc LALA
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 52 - protein, 329 aa, synthetic construct
  REGION 1..329: note = Synthetic: Heavy chain constant chain with Fc LALA without C-terminal lysine
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 53 - protein, 446 aa, synthetic construct
  REGION 1..446: note = Synthetic: full heavy chain hu1H2-03-LALA
EVQLVESGGGLVQPGGSLRLSCAASGFTFSDYGLHWVRQAPGKGLEWVSYISSGSSTVYFADTLKGRFTISRDNAKNSLYLQMNSLRAEDTAVYYCTRGTWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 54 - protein, 445 aa, synthetic construct
  REGION 1..445: note = Synthetic: full heavy chain hu1H2-03-LALA without C-terminal lysine
EVQLVESGGGLVQPGGSLRLSCAASGFTFSDYGLHWVRQAPGKGLEWVSYISSGSSTVYFADTLKGRFTISRDNAKNSLYLQMNSLRAEDTAVYYCTRGTWYFDVWGRGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 55 - protein, 214 aa, synthetic construct
  REGION 1..214: note = Synthetic: full light chain hu1H2-03-LALA
DIQMTQSPSSLSASVGDRVTITCRTSENIHSYLAWYQQKPGKSPQLLVYNAKTLADGVPSRFSGSGSGTDYTLTISSLQPEDFGTFYCQHFWTTSRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 56 - protein, 25 aa, synthetic construct
  REGION 1..25: note = Synthetic: poly-G4S amino acid
  VARIANT 6..15: note = "GGGGS" may or may not be present
GGGGSGGGGSGGGGSGGGGSGGGGS

SEQ ID NO: 57 - protein, 4 aa, synthetic construct
  REGION 1..4: note = Synthetic: linker
GGFG

SEQ ID NO: 58 - protein, 5 aa, synthetic construct
  REGION 1..5: note = Synthetic: LPXTG recognition motif
  VARIANT 3: note = X is any amino acid
LPXTG